(19) United States
(12) Patent Application Publication
(10) Pub. No.: US 2025/0320206 A1
(43) Pub. Date: Oct. 16, 2025

(54) STAT6 DEGRADERS

(71) Applicant: Gilead Sciences, Inc., Foster City, CA (US)

(72) Inventors: Patrick Stephen Johnson, Valby (DK); Geanna Min, Herlev (DK); Thomas Holmstrøm, København S (DK); Sebastian Clementson, Malmö (SE); Pablo Martín-Gago, Frederiksberg (DK); Kristoffer Månsson, Hjärup (SE); Morten Jørgensen, Herlev (DK); Kevin Neil Dack, Kongens Lyngby (DK); Marcel John de Groot, Værløse (DK); Mark David Andrews, Ballerup (DK)

(21) Appl. No.: 19/091,655

(22) Filed: Mar. 26, 2025

(30) Foreign Application Priority Data
Mar. 27, 2024 (EP) EP24167026.4

(51) Int. Cl. C07D 471/04; A61K 31/437

(52) U.S. Cl. CPC C07D 471/04; A61K 31/437

(57) ABSTRACT

The present disclosure provides a compound according to formula (I)

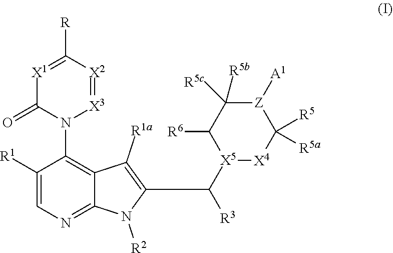

and pharmaceutically acceptable salts, hydrates, or solvates thereof. The disclosure further relates to said compounds for use in therapy, to pharmaceutical compositions comprising said compounds, to methods of treating diseases, e.g. dermal diseases, with said compounds, and to the use of said compounds in the manufacture of medicaments.

### CROSS REFERENCE TO RELATED APPLICATIONS

This application claims priority to European Patent Application No. EP24167026.4, filed Mar. 27, 2024, which is incorporated herein in its entirety for all purposes.

### FIELD

Provided herein are novel compounds and pharmaceutically acceptable salts thereof that modulate STAT6 and pharmaceutical compositions comprising said compounds for use in therapy (e.g., for treating STAT6 associated diseases in a subject in need thereof).

### BACKGROUND

The disclosure relates generally to methods and compounds, and pharmaceutically acceptable salts thereof, for modulating a Signal transducer and activator of transcription 6 (STAT6) protein activity and treating STAT6 associated diseases. The following description sets forth exemplary methods, parameters and the like. It should be recognized, however, that such description is not intended as a limitation on the scope of the present disclosure but is instead provided as a description of exemplary embodiments.

The present disclosure relates to novel bifunctional compounds and pharmaceutically acceptable salts thereof, which may function to recruit STAT6 proteins to E3 ubiquitin ligase for degradation, compositions containing such compounds, and methods and uses thereof. In some embodiments, the present disclosure provides bifunctional compounds and pharmaceutically acceptable salts thereof, which may find utility as modulators of targeted ubiquitination of STAT6 proteins, which may be then degraded and/or otherwise inhibited by the bifunctional compounds as described herein.

Ubiquitin-Proteasome Pathway (UPP) is a pathway that regulates key regulator proteins and degrades proteins, such as misfolded or abnormal proteins. UPP is central to multiple cellular processes, and if defective or imbalanced, it can lead to pathogenesis of a variety of diseases. The attachment of ubiquitin to specific protein substrates can be achieved through the action of E3 ubiquitin ligases.

There are over 600 E3 ubiquitin ligases which facilitate the ubiquitination of different proteins in vivo, which can be generally divided into four families: HECT-domain E3s, U-box E3s, monomeric RING E3s and multi-subunit E3s. See generally Li et al. (PLOS One, 2008, 3, 1487) titled “Genome-wide and functional annotation of human E3 ubiquitin ligases identifies MULAN, a mitochondrial E3 that regulates the organelle's dynamics and signaling.”; Bemdsen et al. (Nat. Struct. Mol. Biol., 2014, 21, 301-307) titled “New insights into ubiquitin E3 ligase mechanism”; Deshaies et al. (Ann. Rev. Biochem., 2009, 78, 399-434) titled “RING domain E3 ubiquitin ligases.”; Spratt et al. (Biochem. 2014, 458, 421-437) titled “RBR E3 ubiquitin ligases: new structures, new insights, new questions.”; and Wang et al. (Nat. Rev. Cancer., 2014, 14, 233-347) titled “Roles of F-box proteins in cancer.”

UPP plays a role in the degradation of short-lived and regulatory proteins important in a variety of basic cellular processes, including regulation of the cell cycle, modulation of cell surface receptors and ion channels, and antigen presentation. The pathway has been implicated in several forms of malignancy, in the pathogenesis of several genetic diseases (including cystic fibrosis, Angelman's syndrome, and Liddle syndrome), in immune surveillance/viral pathogenesis, and in the pathology of muscle wasting. Many diseases are associated with an abnormal UPP and negatively affect cell cycle and division, the cellular response to stress and to extracellular modulators, morphogenesis of neuronal networks, modulation of cell surface receptors, ion channels, the secretory pathway, DNA repair and biogenesis of organelles.

Aberrations in the process have recently been implicated in the pathogenesis of several diseases, both inherited and acquired. These diseases fall into two major groups: (a) those that result from loss of function with the resultant stabilization of certain proteins, and (b) those that result from gain of function, i.e. abnormal or accelerated degradation of the protein target.

The UPP can be used to induce selective protein degradation, including via use of fusion proteins to ubiquitinate target proteins and synthetic small-molecule probes to induce proteasome-dependent degradation. Bifunctional compounds composed of a target protein-binding ligand and an E3 ubiquitin ligase ligand, can induce proteasome-mediated degradation of selected proteins via their recruitment to E3 ubiquitin ligase and subsequent ubiquitination. These drug-like molecules offer the possibility of temporal control over protein expression. Such compounds can be capable of inducing the inactivation of a protein of interest upon addition to cells or administration to an animal or human, and could be useful as biochemical reagents and lead to a new paradigm for the treatment of diseases by removing pathogenic or oncogenic proteins (Crews C, Chemistry & Biology, 2010, 17(6):551-555; Schnnekloth JS Jr., Chembiochem, 2005, 6(1): 40-46).

The signal Transducer and Activator of Transcription 6 (STAT6) belongs to a family of transcription factors (STAT1, STAT2, STAT3, STAT4, STAT5a, STAT5b and STAT6) which may be structurally and/or functionally related, and which may be involved in mediating signalling from multiple cytokine and/or growth factor receptors.

Without being bound by theory, STAT6 can selectively mediate signaling from IL-4 and IL-13 via the IL-4Ra subunit complexing with either the common gamma chain (γc) to form the type I receptor or with the IL-13Rα1 subunit to form a type II receptor. When IL-4 or IL-13 activates the receptor complex, the Janus Kinases (Jak) associated with the cytoplasmic tail of IL-4Ra can be activated and phosphorylate tyrosine residues on the intracellular part of the receptor. This phosphorylation can generate docking site(s) for STAT6, which can bind to the phosphorylated receptor via its Src homology-2 (SH2) domain. This may allow Jak kinases to phosphorylate tyrosine (Y)-641 on STAT6, potentially leading to activation. Activated STAT6 may form a homodimer and relocate to the nucleus and activate gene transcription. The genes transcribed by activated STAT6 can be cell specific and could in general induce Th2 immune responses (Walford and Taylor 2013: STAT6 and lung inflammation. JAK-STAT 2:4, e25301; October/November/December 2013; © 2013 Landes Bioscience).

STAT6 is expressed in numerous cell types including epithelial cells, fibroblasts and immune cells. Without being bound by theory, inhibition of STAT6 activity can inhibit the IL-4 and IL-13 mediated effects in cells, including the differentiation of T-cells into Th2 cells and B-cell class shift into IgE and IgG1 producing cells (Walford). In epidermal keratinocytes, a STAT6 inhibitor could inhibit the secretion of pro-inflammatory chemokines and revert the cytokine-induced inhibition of barrier function proteins such as filaggrin (Tollenaire et al 2017: Skin Barrier and Inflammation Genes Associated with Atopic Dermatitis are Regulated by Interleukin-13 and Modulated by Tralokinumab In vitro. Acta Derm Venereol 2021; 101: adv00447.

Antibodies targeting Th2 immune responses, such as the IL-4Ra (dupilumab) or IL-13 (tralokinumab, lebrikizumab), have shown efficacy in a number of Th2-driven diseases. Targeting STAT6 with a small molecule inhibitor allows for targeting the same pathway by an oral or dermal administration route and may have efficacy in diseases where dupilumab has shown effect. A compound antagonizing STAT6 could, therefore, have utility in treating conditions characterized by Th2-mediated inflammation such as atopic dermatitis, prurigo nodularis, Bullous phemphigoid, asthma, chronic rhinosinusitis with nasal polyposis, urticaria (such as chronic spontaneous urticaria), rhinitis, eosinophilic esophagitis, food allergy, diffuse cutaneous systemic sclerosis, alopecia areata and/or COPD.

STAT6 is also involved in differentiation and activity of M2 macrophages, including the tumor-associated macrophages (TAMs) in solid tumors. TAMs protect the tumor from immune attack by inducing a pro-tumor immunosuppressive environment. TAMs may inhibit T-cell proliferation, block migration of CD8 T-cells into the tumor and recruit Tregs into the tumor microenvironment (Karpathiou et al 2021: STAT6: A review of a signaling pathway implicated in various diseases with a special emphasis in its usefulness in pathology. Pathology—Research and Practice 223 (2021) 153477).

In addition, IL-13 may act as a growth factor for some tumors and for some tumors gain-of-function mutations in STAT6 have been described as oncogenes (Karpathiou et al 2021: STAT6: A review of a signaling pathway implicated in various diseases with a special emphasis in its usefulness in pathology. Pathology—Research and Practice 223 (2021) 153477).

Together these data suggests that a STAT6 degraders may treat different cancers such as lymphomas, non-small cell lung cancer and solid fibrous cancers either as a stand-alone treatment or in combination with other anticancer drugs such as check point inhibitors.

Although various antibodies against IL-4R or IL-13 are approved for medical use, there are currently no approved, orally available degraders of STAT6.

Therefore, there remains a continuous need to develop degraders of STAT-6, particularly small molecules suitable for oral administration.

In addition, some patients may be treated by topical application of degraders of STAT-6. This can be particularly suitable, for example, for patients with skin lesions that are readily accessible and limited to areas on the body surface. Topical treatment may also be prescribed for certain patients who could benefit from avoiding systemic modulation of the STAT-6 pathway, for example when undergoing treatment for infections or gastrointestinal problems.

### SUMMARY

The inventors have surprisingly found that the novel compounds and salts thereof as described in the present disclosure exhibit modulating effects on the STAT-6 signalling pathway.

For example, the compounds and pharmaceutically acceptable salts thereof as described herein may be beneficial in preventing, treating or ameliorating a variety of diseases which involve up-regulation or de-regulation of STAT-6.

Furthermore, the compounds and pharmaceutically acceptable salts thereof as described herein have advantageous properties such as high metabolic stability, membrane permeability and/or solubility that make them particularly suitable for oral administration.

Moreover, some patients may be treated by topical application of degraders of STAT-6. This can be particularly suitable, for example, for patients with skin lesions that are readily accessible and limited to areas on the body surface. Topical treatment may also be prescribed for certain patients who could benefit from avoiding systemic modulation of the STAT-6 pathway, for example when undergoing treatment for infections or gastrointestinal problems. Thus, some aspects of the present disclosure relate to methods for the topical application of the compounds and salts thereof as described herein.

Accordingly, in some embodiments, the present disclosure provides a compound according to formula (I)

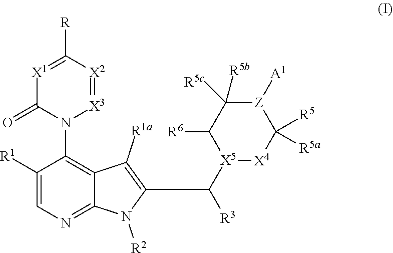

wherein:
A¹ is selected from

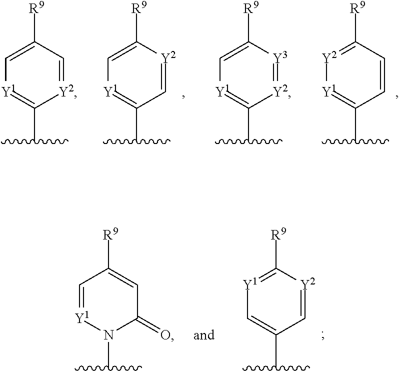

R⁹ is -A-L-LBM;
X¹ is selected from N and CR¹¹; X² is selected from N and CR¹²; and X³ is selected from N and CR¹³, provided that only one of X¹, X² and X³ may be N;
X⁴ is selected from CR⁴R⁴ and CO;
X⁵ is selected from N and oxidized N;
Y¹ is selected from N and CR⁷;
Y² is selected from N and CR⁸;
Y³ is selected from N and CR¹⁷;
Z is selected from N and CR¹⁰;
R is NHR⁰;
R⁰ is selected from hydrogen, C₁₋₄alkyl, C₃₋₄ cycloalkyl, C₃₋₄ cycloalkyl-C₁₋₄alkyl, phenyl-C₁₋₄alkyl, halo-C₁₋₄alkyl, —(CH₂)ₙ—O—C₁₋₄alkyl, —(CH₂)ₙ—CO—R′, and —(CH₂)ₙ—CO₂R′, wherein said phenyl is optionally substituted one or more times with a substituents independently selected from halogen, C₁₋₄alkyl, halo-C₁₋₄alkyl, C₃₋₄cycloalkyl, hydroxy, C₁₋₄alkoxy, cyano, —NR′R″, —CONR′R″, and —CO₂R′, and wherein R′ and R″ are each independently selected from hydrogen and C₁₋₄alkyl, and n is 0, 1 or 2;
R¹ and R^{1a} are independently selected from hydrogen, fluoro, and C₁₋₄alkyl;
R² is selected from hydrogen, C₁₋₅alkyl, —SO₂—C₁₋₄alkyl and deuterated C₁₋₄alkyl, wherein said C₁₋₅alkyl may optionally be substituted one or more times with halogen;
R³ is selected from hydrogen, C₁₋₄alkyl, and deuterated C₁₋₄alkyl;
each of R⁴ and R⁶ is independently selected from hydrogen, C₁₋₄alkyl, and C₁₋₄ alkoxy, said C₁₋₄alkyl and C₁₋₄alkoxy may optionally be substituted with one or more halogen;
R⁵ is selected from hydrogen, halogen, C₁₋₄alkyl and C₁₋₄alkoxy, wherein said C₁₋₄alkyl and C₁₋₄alkoxy may optionally be substituted with one or more halogen;
R¹⁰ is selected from hydrogen and fluoro;
or R⁵ and R¹⁰ together form a bond between the two carbons to which they are attached; R^{5a} is hydrogen;
or R⁵ and R^{5a} are both fluoro or R⁵ and R^{5a} together with the atom attached thereto form a CO group;
R^{5b} and R^{5c} are each independently selected from hydrogen and fluoro;
R⁷, R⁸, and R¹⁷ are each independently selected from hydrogen, halogen, cyano, C₁₋₄alkyl and C₁₋₄alkoxy, and C₃₋₄cycloalkyl, wherein said C₁₋₄alkyl and C₁₋₄alkoxy may optionally be substituted with one or more halogen;
R¹¹, R¹², and R¹³ are each independently selected from hydrogen, halogen, C₁₋₄alkyl, C₃₋₄ cycloalkyl, C₁₋₄alkoxy, hydroxy, cyano, —NR′R″, —CONR′R″, —CO₂R′, and —CO—(CH₂)ₘOH, wherein said C₁₋₄alkyl is optionally substituted one or more times with a substituent independently selected from halogen, hydroxy and C₁₋₄alkoxy, and wherein m is 1, 2 or 3 and wherein R′ and R″ are each independently selected from hydrogen and C₁₋₄alkyl; or
R¹¹ and R⁰ together with the atoms attached thereto form a 5-6 membered heteroaromatic or heterocyclic ring containing one or two N atoms, wherein said 5-6 membered heteroaromatic ring or heterocyclic ring containing one or two N atoms is optionally substituted one or more times with a substituent independently selected from halogen, C₁₋₄alkyl, halo-C₁₋₄alkyl, C₃₋₄ cycloalkyl, hydroxy, C₁₋₄alkoxy, cyano, —COCF₃, —NR′R″, —CONR′R″, and —CO₂R′, wherein said C₁₋₄alkyl may optionally be substituted one or more times with a substituent independently selected from halogen and C₁₋₄alkoxy, and wherein R′ and R″ are each independently selected from hydrogen and C₁₋₄alkyl;
R¹² and R¹³ are each independently selected from hydrogen, halogen, C₁₋₄alkyl, C₃₋₄ cycloalkyl, C₁₋₄alkoxy, hydroxy, cyano, —NR′R″, —CONR′R″, —CO₂R′, —CO—CO₂R′ and —CO—(CH₂)ₘOH, wherein said C₁₋₄alkyl is optionally substituted one or more times with a substituent independently selected from halogen, hydroxy and C₁₋₄alkoxy, and wherein m is 1, 2 or 3 and wherein R′ and R″ are each independently selected from hydrogen and C₁₋₄alkyl;
A is CO;
L is selected from

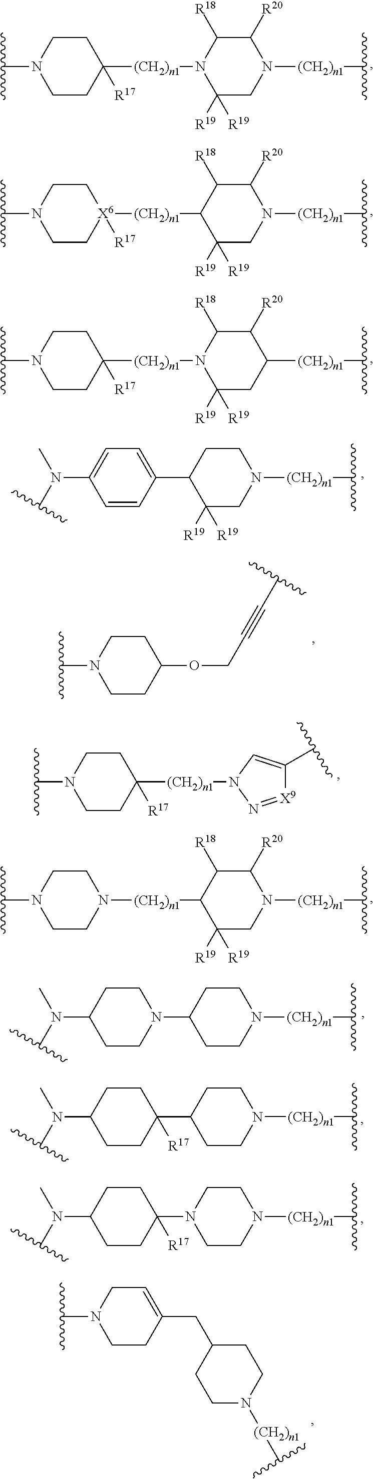

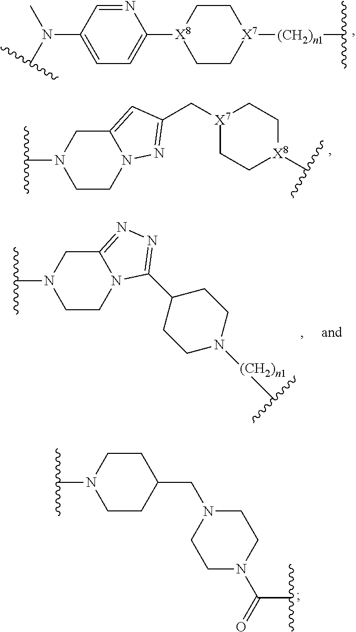

wherein n1 is selected from 0 and 1;
n2 is selected from 1 and 2;
R¹⁷ is selected from hydrogen and fluoro;
R¹⁸ is selected from hydrogen, fluoro, C₁₋₄alkyl wherein said C₁₋₄alkyl may optionally be substituted one or more times with fluoro or C₁₋₄alkoxy;
R¹⁹ is independently selected from hydrogen, C₁₋₄alkyl and fluoro;
R²⁰ is selected from hydrogen and C₁₋₄alkyl;
X⁶ is C;
X⁷ is CR²⁴ or N;
R²⁴ is selected from hydrogen and hydroxy;
X⁸ and X⁹ are independently CH or N; and
LBM is

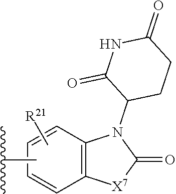

wherein X⁷ is O, NR²², or CR²³R²³;
R²¹ is selected from hydrogen, fluoro, and chloro;
R²² is selected from hydrogen, C₁₋₄alkyl, deuterated C₁₋₄alkyl, and C₃₋₄ cycloalkyl, wherein said C₁₋₄alkyl may optionally be substituted one or more times with fluoro, hydroxy, C₁₋₄alkoxy, or C₃₋₄ cycloalkyl;
each R²³ is independently selected from hydrogen, halogen, and C₁₋₄alkyl; or
both R²³ together with the carbon they are attached to form a C₃₋₆ cycloalkyl or 3-6 membered heterocyclic ring, wherein the heterocyclic ring contains one to two heteroatoms selected from O;
or a pharmaceutically acceptable salt or stereoisomer thereof.

In some embodiments, the present disclosure provides a compound according to formula (I′)

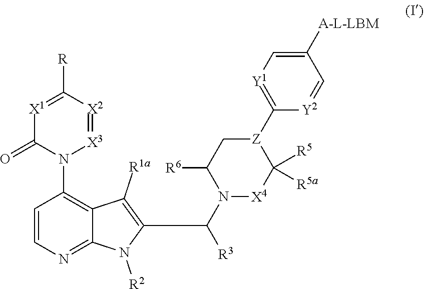

Wherein:
X¹ is selected from N and CR¹¹; X² is selected from N and CR¹²; and X³ is selected from N and CR¹³, provided that only one of X¹, X² and X³ may be N;
X⁴ is CR⁴R⁴ or CO;
Y¹ is selected from N and CR⁷;
Y² is selected from N and CR⁸;
Z is selected from N and CR¹⁰;
R is NHR⁰;
R⁰ is selected from hydrogen, C₁₋₄alkyl, C₃₋₄ cycloalkyl, C₃₋₄ cycloalkyl-C₁₋₄alkyl, phenyl-C₁₋₄alkyl, halo-C₁₋₄alkyl, —(CH₂)ₙ—O—C₁₋₄alkyl, —(CH₂)ₙ—CO—R′, and —(CH₂)ₙ—CO₂R′, wherein said phenyl is optionally substituted one or more times with a substituent independently selected from halogen, C₁₋₄alkyl, halo-C₁₋₄alkyl, C₃₋₄cycloalkyl, hydroxy, C₁₋₄alkoxy, cyano, —NR′R″, —CONR′R″, and —CO₂R′, and wherein R′ and R″ are each independently selected from hydrogen and C₁₋₄alkyl, and n is 0, 1, or 2;
R^{1a} is hydrogen;
R² is selected from hydrogen, C₁₋₄alkyl, and deuterated C₁₋₄alkyl; wherein said C₁₋₄alkyl may optionally be substituted one or more times with halogen;
R³ is selected from hydrogen, C₁₋₄alkyl, and deuterated C₁₋₄alkyl;
R⁴ and R⁶ are independently selected from hydrogen and C₁₋₄alkyl, wherein said C₁₋₄alkyl may optionally be substituted with one or more halogens;
R⁵ is selected from hydrogen, halogen, C₁₋₄alkyl, and C₁₋₄alkoxy, wherein said C₁₋₄alkyl and C₁₋₄alkoxy may optionally be substituted with one or more halogen;
R¹⁰ is selected from hydrogen and fluoro;
or R⁵ and R¹⁰ together form a bond between the two carbons to which they are attached;
R^{5a} is hydrogen; or R⁵ and R^{5a} are both fluoro;
R⁷ and R⁸ are independently selected from hydrogen, halogen, C₁₋₄alkyl, C₃₋₄ cycloalkyl, and C₁₋₄alkoxy, wherein said C₁₋₄alkyl and C₁₋₄alkoxy may independently optionally be substituted with one or more halogen;
R¹¹ is selected from hydrogen, halogen, C₁₋₄alkyl, C₃₋₄cycloalkyl, C₁₋₄alkoxy, hydroxy, cyano, —NR′R″, —CONR′R″, —CO₂R′, —CO—(CH₂)ₘOH, wherein said C₁₋₄alkyl is optionally substituted one or more times with a substituent independently selected from halogen, hydroxy and C₁₋₄alkoxy, and wherein m is 1, 2 or 3 and wherein R′ and R″ are each independently selected from hydrogen and C₁₋₄alkyl; or
R¹¹ and R⁰ together with the atoms attached thereto may form a 5-6 membered heterocyclic or heteroaromatic ring containing one to two heteroatoms selected from N, wherein said 5-6 membered heterocyclic or heteroaromatic ring containing one to two heteroatoms selected from N is optionally substituted one or more times with a substituent independently selected from halogen, C₁₋₄alkyl, halo-C₁₋₄alkyl, C₃₋₄cycloalkyl, hydroxy, C₁₋₄alkoxy, cyano, —NR′R″, —CONR′R″ and —CO₂R′, wherein said C₁₋₄alkyl may optionally be substituted one or more times with a substituent independently selected from halogen and C₁₋₄alkoxy, and wherein R′ and R″ are each independently selected from hydrogen and C₁₋₄alkyl;
R¹² and R¹³ are independently selected from hydrogen, halogen, C₁₋₄alkyl, C₃₋₄ cycloalkyl, C₁₋₄alkoxy, hydroxy, cyano, —NR′R″, —CONR′R″, —CO₂R′, —CO—CO₂R′ and —CO—(CH₂)ₘOH, wherein said C₁₋₄alkyl is optionally substituted one or more times with a substituent independently selected from halogen, hydroxy, and C₁₋₄alkoxy, and wherein m is 1, 2 or 3 and wherein R′ and R″ are each independently selected from hydrogen and C₁₋₄alkyl;
A is CO;
L is selected from

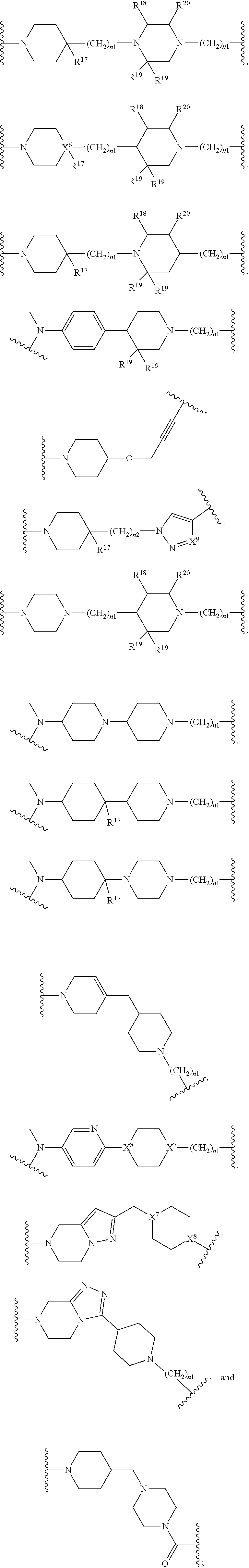

wherein n1 is selected from 0 and 1;
n2 is selected from 1 and 2;
R¹⁷ is selected from hydrogen and fluoro;
R¹⁸ is selected from hydrogen, fluoro, C₁₋₄alkyl wherein said C₁₋₄alkyl may optionally be substituted one or more times with fluoro or C₁₋₄alkoxy;
R¹⁹ is independently selected from hydrogen, C₁₋₄alkyl and fluoro;
R²⁰ is selected from hydrogen and C₁₋₄alkyl;
X⁶ is C;
X⁷ is CR²⁴ or N;
R²⁴ is selected from hydrogen and hydroxy;
X⁸ and X⁹ are independently CH or N; and
LBM is

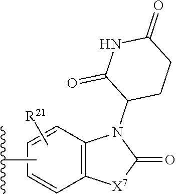

wherein X⁷ is O, NR²², or CR²³R²³;
R²¹ is selected from hydrogen, fluoro, and chloro;
R²² is selected from hydrogen, C₁₋₄alkyl, deuterated C₁₋₄alkyl, and C₃₋₄ cycloalkyl, wherein said C₁₋₄alkyl may optionally be substituted one or more times with fluoro, hydroxy, C₁₋₄ alkoxy, or C₃₋₄ cycloalkyl;
each R²³ is independently selected from hydrogen, halogen, and C₁₋₄alkyl; or
both R²³ together with the carbon they are attached to form a C₃₋₆ cycloalkyl or 3-6 membered heterocyclic ring, wherein the heterocyclic ring contains one to two heteroatoms selected from O;
or a pharmaceutically acceptable salt or stereoisomer thereof.

In another embodiment, the present disclosure provides a pharmaceutical composition comprising a compound of the present disclosure, or a pharmaceutically acceptable salt or stereoisomer thereof, and a pharmaceutically acceptable excipient.

In another embodiment, the present disclosure provides a method of treating an immune mediated disease or condition in a subject in need thereof, the method comprising administering to the subject a therapeutically effective amount of a compound of the present disclosure, or a pharmaceutically acceptable salt or stereoisomer thereof, or a pharmaceutical composition of the present disclosure.

In another embodiment, the present disclosure provides a method of modulating a signal transducer and activator of transcription 6 (STAT6) protein activity in a subject in need thereof, the method comprising administering to the subject a therapeutically effective amount of a compound of the present disclosure, or a pharmaceutically acceptable salt or stereoisomer thereof, or a pharmaceutical composition of the present disclosure.

In another embodiment, the present disclosure provides a method of treating a disease or condition mediated by a signal transducer and activator of transcription 6 (STAT6) protein activity in a subject in need thereof, the method comprising administering to the subject a therapeutically effective amount of a compound of the present disclosure, or a pharmaceutically acceptable salt or stereoisomer thereof, or a pharmaceutical composition of the present disclosure.

In another embodiment, the present disclosure provides a method of treating a disease or condition mediated by interleukin 4 (IL-4) or interleukin 3 (IL-3) in a subject in need thereof, the method comprising administering to the subject a therapeutically effective amount of a compound of the present disclosure, or a pharmaceutically acceptable salt or stereoisomer thereof, or a pharmaceutical composition of the present disclosure.

In one embodiment the disclosure provides a method of preventing, treating or ameliorating a diseases characterized by Th2-mediated inflammation.

In another embodiment, the present disclosure provides a method for manufacturing a medicament for treating a disease or condition mediated by a signal transducer and activator of transcription 6 (STAT6) protein activity in a subject in need thereof, characterized in that a compound of the present disclosure, or a pharmaceutically acceptable salt or stereoisomer thereof, is used.

In another embodiment, the present disclosure provides a method for manufacturing a medicament for the treatment of a disease or condition mediated by a signal transducer and activator of transcription 6 (STAT6) protein in a subject.

In some embodiments, the present disclosure provides a compound of the present disclosure, or a pharmaceutically acceptable salt or stereoisomer thereof, for use in treating a disease or condition mediated by a signal transducer and activator of transcription 6 (STAT6) protein in a subject in need thereof.

In some embodiments, the present disclosure provides the compound of the present disclosure, or a pharmaceutically acceptable salt or stereoisomer thereof, for use in therapy.

### BRIEF DESCRIPTION OF THE DRAWINGS

FIG. 1 shows the results of anHiBit Assay, dose-response curve for Examples 3u208, 3u246, 3u118, 3u119, 3u120, and 3u74 (where percent degradation is plotted vs. test compound concentration in M on a log-scale).

FIG. 2 shows data from the human whole blood eotaxin-3 assay for Examples 3u208, 3u246, 3u118, 3u119, 3u120, and 3u74 (where the effect in % is plotted against the test concentration in M on a log scale).

### DETAILED DESCRIPTION

### Definitions

Whenever the compound of formula (I) is mentioned herein it should be understood that the compound of formula (I′), (I″), (II), (II′), (III), (IV), (IV′), (VII), (VIII), (VIII′), (IX), (IXa), and (IXb), and other subformulas described herein, are subgroups of the compound of formula (I) and that a statement related to the compound of formula (I) relates equally well to its subgroups.

The prefix “Cᵤ₋ᵥ” indicates that the following group has from u to v carbon atoms. For example, “C₁₋₄ alkyl” indicates that the alkyl group has from 1 to 4 carbon atoms.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art. The term “C₁₋₄alkyl” is used herein to refer to hydrocarbon radical obtained when one hydrogen atom is removed from a branched or linear hydrocarbon. Said alkyl comprises (1-4) carbon atoms, 1-3 carbon atoms, 2-3 carbon atoms or 1-2 carbon atoms. The term includes the subclasses normal alkyl (n-alkyl), secondary and tertiary alkyl, such as methyl, ethyl, n-propyl, isopropyl, n-butyl, isobutyl, sec-butyl, and tert-butyl.

The term “C₁₋₄alkyl” is used herein to refer to hydrocarbon radical obtained when one hydrogen atom is removed from a branched or linear hydrocarbon. Said alkyl comprises (1-4) carbon atoms, 1-3 carbon atoms, 2-3 carbon atoms or 1-2 carbon atoms. The term includes the subclasses normal alkyl (n-alkyl), secondary and tertiary alkyl, such as methyl, ethyl, n-propyl, isopropyl, n-butyl, isobutyl, sec.-butyl, and tert.-butyl.

The term “(C₁-C₄)alkoxy” is used herein to refer to a radical of the formula —OR′, wherein R′ is (C₁-C₄)alkyl as indicated herein, wherein the (C₁-C₄)alkyl group is appended to the parent molecular moiety through an oxygen atom, e.g. methoxy (—OCH₃), and ethoxy (—OCH₂CH₃).

The term “cyano” is used herein to refer to a —CN group attached to the parent molecular moiety through the carbon atom.

The term “(C₃-C₄)cycloalkyl” is used herein to refer to a saturated (C₃-C₄)cycloalkane hydrocarbon radical, comprising 3-4 carbon atoms, e.g. cyclopropyl, or cyclobutyl.

The term “halogen” or “halo” is used herein to refer to chloro, bromo, fluoro, or iodo. In some embodiments, halogen is chloro, bromo, or fluoro.

“Aromatic ring” or “aryl” refers to an aromatic carbocyclic group having a single ring (e.g., monocyclic) or multiple rings (e.g., bicyclic or tricyclic) including fused systems. An aryl may have 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include, e.g., phenyl, naphthyl, fluorenyl, and anthryl. Aryl, however, does not encompass or overlap in any way with heteroaryl defined below. If one or more aryl groups are fused with a heteroaryl, the resulting ring system is heteroaryl regardless of point of attachment. If one or more aryl groups are fused with a heterocyclyl, the resulting ring system is heterocyclyl regardless of point of attachment. If one or more aryl groups are fused with a cycloalkyl, the resulting ring system is cycloalkyl regardless of point of attachment.

“Heteroaromatic ring” or “heteroaryl” refers to an aromatic group having a single ring, multiple rings or multiple fused rings, with one or more ring heteroatoms independently selected from nitrogen, oxygen, and sulfur unless specified otherwise. A heteroaryl may include 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen, and sulfur. In certain instances, heteroaryl includes 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen, and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, thiophenyl (i.e., thienyl), triazolyl, tetrazolyl, and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclic ring” or “heterocyclyl” refers to a saturated or partially unsaturated cyclic alkyl group, with one or more ring heteroatoms independently selected from nitrogen, oxygen, and sulfur, unless specified otherwise. The term “heterocyclyl” includes heterocycloalkenyl groups (i.e., the heterocyclyl group having at least one double bond), bridged-heterocyclyl groups, fused-heterocyclyl groups, and spiro-heterocyclyl groups. A heterocyclyl may be a single ring or multiple rings wherein the multiple rings may be fused, bridged, or spiro, and may comprise one or more (e.g., 1 to 3) oxo (═O) or N-oxide (—O—) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to a cycloalkyl, an aryl, or heteroaryl ring, regardless of the attachment to the remainder of the molecule. A heterocyclyl may have 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur, or oxygen. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiomorpholinyl, thiamorpholinyl, 1-oxo-thiomorpholinyl, and 1,1-dioxo-thiomorpholinyl.

The term “halo-C₁₋₄alkyl” or “halo-C₁₋₄alkoxy” is used herein to refer to an “C₁₋₄alkyl” or “C₁₋₄ alkoxy” group respectively as defined above in which one or more hydrogen atoms have been replaced by halogen (e.g. fluorine). Examples of “halo-C₁₋₄alkyl” or “halo-C₁₋₄alkoxy” include —CHF₂, —CF₃, —CH₂CF₃, —CF₂CF₃ or —OCF₃.

The term “deuterated C₁₋₄alkyl” is used herein to refer to an “C₁₋₄alkyl” group in which one or more hydrogen atoms have been replaced by deuterium. Examples of “deuterated C₁₋₄alkyl” include —CH₂D₂, —CHD₂ or —CD3.

The term “C₁₋₄alkoxyl-C₁₋₄alkyl” is used herein the refer to “C₁₋₄alkyl” group in which one hydrogen atom have been replaced by (C₁-C₄)alkoxy. Examples of “C₁₋₄alkoxyl-C₁₋₄alkyl” “include methoxymethyl or methoxyethyl.

The term “(C₃-C₄)cycloalkyl-C₁₋₄alkyl” is used herein the refer to “C₁₋₄alkyl” group in which one hydrogen atom have been replaced by (C₃-C₄)cycloalkyl. Examples of “(C₃-C₄)cycloalkyl-C₁₋₄alkyl” include cyclopropylmethyl.

The term “phenyl-C₁₋₄alkyl” is used herein the refer to “C₁₋₄alkyl” group in which one hydrogen atom has been replaced by phenyl. Examples of “phenyl-C₁₋₄alkyl” include benzyl.

If substituents are described as being independently selected from a group, each substituent is selected independent of the other. Each substituent may therefore be identical or different from the other substituent(s).

The term “optionally substituted” means “unsubstituted or substituted”. In some embodiments, formulas described herein encompasses compounds containing the specified optional substituent(s) as well as compounds that do not contain the optional substituent(s).

In certain embodiments, as used herein, the phrase “one or more” refers to one to five. In certain embodiments, as used herein, the phrase “one or more” refers to one to three.

As used herein whenever a molecular drawing of a substituent contains an arrow—the arrow indicates the bond attaching the substituent to the rest of the molecule.

The term “pharmaceutically acceptable salt” is intended to indicate non-toxic salts a free base form of a compound that possesses the desired pharmacological activity of the free base. These salts may be derived from an appropriate basic moiety, with a suitable inorganic or organic acid, such as hydrochloric, hydrobromic, hydroiodic, sulfuric, nitric, phosphoric, formic, acetic, 2,2-dichloroacetic, adipic, ascorbic, L-aspartic, L-glutamic, galactaric, lactic, maleic, L-malic, phthalic, citric, propionic, benzoic, glutaric, gluconic, D-glucuronic, methanesulfonic, salicylic, succinic, malonic, tartaric, benzenesulfonic, ethane-1,2-disulfonic, 2-hydroxyethanesulfonic acid, toluenesulfonic, sulfamic or fumaric acid.

In some embodiments, pharmaceutically acceptable salts of compounds of formula (I) comprising an acidic moiety may also be prepared by reaction with a suitable base such as sodium hydroxide, potassium hydroxide, magnesium hydroxide, calcium hydroxide, zinc hydroxide, barium hydroxide, ammonia or the like, or suitable non-toxic amines, such as lower alkylamines (such as diethylamine, tetraalkylammonium hydroxide), hydroxy-lower alkylamines (such as diethanolamine, 2-(diethylamino)-ethanol, ethanolamine, triethanolamine, tromethamine, deanol), cycloalkylamines, ethylene diamine, or benzylamines, (such as benethamine and benzathine), betaine, choline hydroxide, N-methyl-glucamine, hydrabamine, 1H-imidazole, 4-(2-hydroxyethyl)-morpholine, piperazine, 1-(2-hydroxyethyl)-pyrrolidine, L-arginine or L-lysine. Further examples of pharmaceutical acceptable salts are listed in Berge, S. M.; J. Pharm. Sci.; (1977), 66(1), 1-19, and Stahl, P. H. and in Wermuth, C. G, Handbook of Pharmaceutical Salts, Properties, Selection and Use, 2^{nd} Edition, Wiley-VCH, 2011 both of which are incorporated herein by reference.

The term “solvate” is intended to indicate a species formed by interaction between a compound, e.g. a compound of formula (I) or a pharmaceutically acceptable salt thereof, and a solvent, e.g. alcohol, glycerol or water, wherein said species are in a crystalline form. When water is the solvent, said species is referred to as a hydrate.

The term “treatment” as used herein means the management and care of a patient for the purpose of combating a disease, disorder or condition. The term is intended to include the delaying of the progression of the disease, disorder or condition, the amelioration, alleviation or relief of symptoms and complications, and/or the cure or elimination of the disease, disorder or condition. The term may also include prevention of the condition, wherein prevention is to be understood as the management and care of a patient for the purpose of combating the disease, condition or disorder and includes the administration of the active compounds to prevent the onset of the symptoms or complications. Nonetheless, prophylactic (preventive) and therapeutic (curative) treatments are two separate aspects.

“Administering” refers to oral administration, administration as a suppository, topical contact (e.g., transdermal), parenteral, intravenous, intraperitoneal, intramuscular, intralesional, intranasal, inhaled, intradermal, and/or subcutaneous administration, intrathecal administration, and/or the implantation of a slow-release device e.g., a mini-osmotic pump, to the subject. The administration can be carried out according to a schedule specifying frequency of administration, dose for administration, and other factors.

“Co-administration” as used herein refers to administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some embodiments, a unit dose of a compound of the present disclosure is administered first, followed within seconds or minutes by administration of a unit dose of one or more additional therapeutic agents. Alternatively, in other embodiments, a unit dose of one or more additional therapeutic agents is administered first, followed by administration of a unit dose of a compound of the present disclosure within seconds or minutes. In some embodiments, a unit dose of a compound of the present disclosure is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a compound of the present disclosure. Co-administration of a compound disclosed herein with one or more additional therapeutic agents generally refers to simultaneous or sequential administration of a compound disclosed herein and one or more additional therapeutic agents, such that therapeutically effective amounts of each agent are present in the body of the patient.

### Compounds

Provided herein are compounds that modulate the activity of STAT6.

In one embodiment the disclosure provides a compound of formula (I)
wherein:

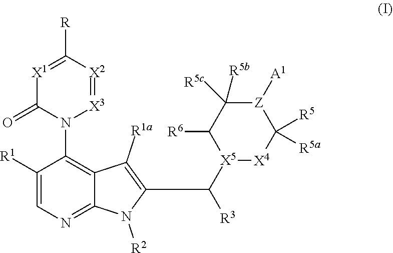

A¹ is selected from

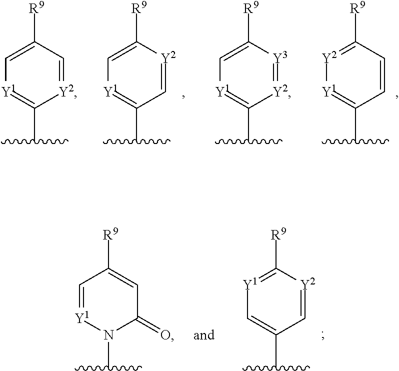

R⁹ is -A-L-LBM;
X¹ is selected from N and CR¹¹; X² is selected from N and CR¹²; and X³ is selected from N and CR¹³, provided that only one of X¹, X² and X³ may be N;
X⁴ is selected from CR⁴R⁴ and CO;
X⁵ is selected from N and oxidized N;
Y¹ is selected from N and CR⁷;
Y² is selected from N and CR⁸;
Y³ is selected from N and CR¹⁷;
Z is selected from N and CR¹⁰;
R is NHR⁰;
R⁰ is selected from hydrogen, C₁₋₄alkyl, C₃₋₄ cycloalkyl, C₃₋₄ cycloalkyl-C₁₋₄alkyl, phenyl-C₁₋₄alkyl, halo-C₁₋₄alkyl, —(CH₂)ₙ—O—C₁₋₄alkyl, —(CH₂)ₙ—CO—R′, and —(CH₂)ₙ—CO₂R′, wherein said phenyl is optionally substituted one or more times with a substituents independently selected from halogen, C₁₋₄alkyl, halo-C₁₋₄alkyl, C₃₋₄cycloalkyl, hydroxy, C₁₋₄alkoxy, cyano, —NR′R″, —CONR′R″, and —CO₂R′, and wherein R′ and R″ are each independently selected from hydrogen and C₁₋₄alkyl, and n is 0, 1 or 2;
R¹ and R^{1a} are independently selected from hydrogen, fluoro, and C₁₋₄alkyl;
R² is selected from hydrogen, C₁₋₅alkyl, —SO₂—C₁₋₄alkyl and deuterated C₁₋₄alkyl, wherein said C₁₋₅alkyl may optionally be substituted one or more times with halogen;
R³ is selected from hydrogen, C₁₋₄alkyl, and deuterated C₁₋₄alkyl;
each of R⁴ and R⁶ is independently selected from hydrogen, C₁₋₄alkyl, and C₁₋₄ alkoxy, said C₁₋₄alkyl and C₁₋₄alkoxy may optionally be substituted with one or more halogen;
R⁵ is selected from hydrogen, halogen, C₁₋₄alkyl and C₁₋₄alkoxy, wherein said C₁₋₄alkyl and C₁₋₄alkoxy may optionally be substituted with one or more halogen;
R¹⁰ is selected from hydrogen and fluoro;
or R⁵ and R¹⁰ together form a bond between the two carbons to which they are attached;
R^{5a} is hydrogen;
or R⁵ and R^{5a} are both fluoro or R⁵ and R^{5a} together with the atom attached thereto form a CO group;
R^{5b} and R^{5c} are each independently selected from hydrogen and fluoro;
R⁷, R⁸, and R¹⁷ are each independently selected from hydrogen, halogen, cyano, C₁₋₄alkyl and C₁₋₄alkoxy, and C₃₋₄cycloalkyl, wherein said C₁₋₄alkyl and C₁₋₄alkoxy may optionally be substituted with one or more halogen;
R¹¹, R¹², and R¹³ are each independently selected from hydrogen, halogen, C₁₋₄alkyl, C₃₋₄ cycloalkyl, C₁₋₄ alkoxy, hydroxy, cyano, —NR′R″, —CONR′R″, —CO₂R′, and —CO—(CH₂)ₘOH, wherein said C₁₋₄ alkyl is optionally substituted one or more times with a substituent independently selected from halogen, hydroxy and C₁₋₄alkoxy, and wherein m is 1, 2 or 3 and wherein R′ and R″ are each independently selected from hydrogen and C₁₋₄alkyl; or
R¹¹ and R⁰ together with the atoms attached thereto form a 5-6 membered heteroaromatic or heterocyclic ring containing one or two N atoms, wherein said 5-6 membered heteroaromatic ring or heterocyclic ring containing one or two N atoms is optionally substituted one or more times with a substituent independently selected from halogen, C₁₋₄alkyl, halo-C₁₋₄alkyl, C₃₋₄ cycloalkyl, hydroxy, C₁₋₄alkoxy, cyano, —COCF₃, —NR′R″, —CONR′R″, and —CO₂R′, wherein said C₁₋₄alkyl may optionally be substituted one or more times with a substituent independently selected from halogen and C₁₋₄alkoxy, and wherein R′ and R″ are each independently selected from hydrogen and C₁₋₄alkyl; and
R¹² and R¹³ are each independently selected from hydrogen, halogen, C₁₋₄alkyl, C₃₋₄ cycloalkyl, C₁₋₄alkoxy, hydroxy, cyano, —NR′R″, —CONR′R″, —CO₂R′, —CO—CO₂R′ and —CO—(CH₂)ₘOH, wherein said C₁₋₄alkyl is optionally substituted one or more times with a substituent independently selected from halogen, hydroxy and C₁₋₄alkoxy, and wherein m is 1, 2 or 3 and wherein R′ and R″ are each independently selected from hydrogen and C₁₋₄alkyl;
A is CO;
L is selected from

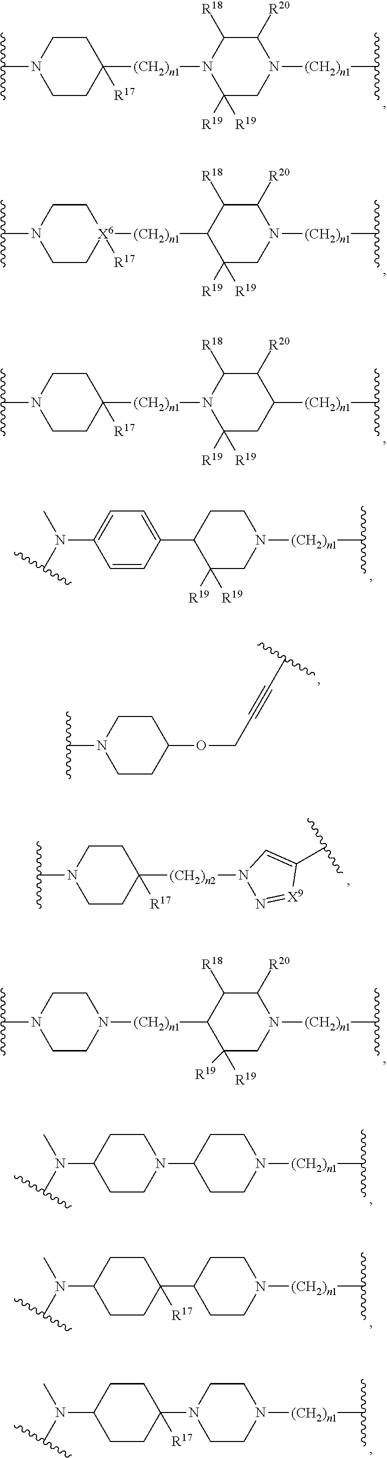

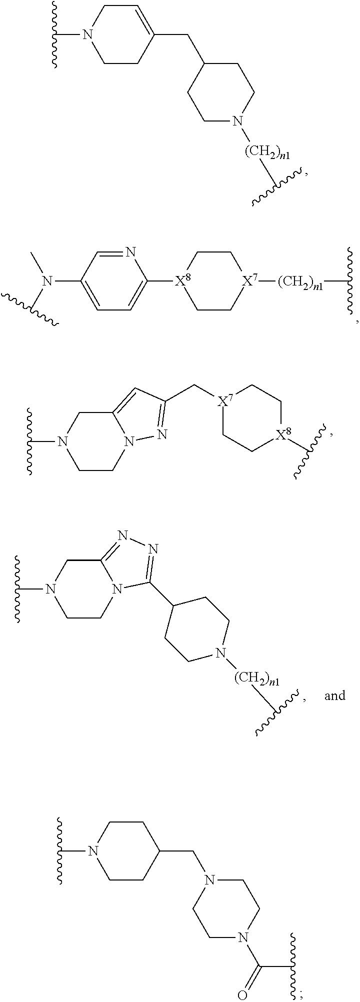

wherein n1 is selected from 0 and 1;
n2 is selected from 1 and 2;
R¹⁷ is selected from hydrogen and fluoro;
R¹⁸ is selected from hydrogen, fluoro, C₁₋₄alkyl wherein said C₁₋₄alkyl may optionally be substituted one or more times with fluoro or C₁₋₄alkoxy;
R¹⁹ is independently selected from hydrogen, C₁₋₄alkyl and fluoro;
R²⁰ is selected from hydrogen and C₁₋₄alkyl;
X⁶ is C;
X⁷ is CR²⁴ or N;
R²⁴ is selected from hydrogen and hydroxy;
X⁸ and X⁹ are independently CH or N; and
LBM is

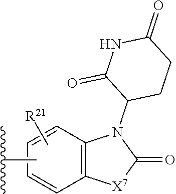

wherein X⁷ is O, NR²², or CR²³R²³
R²¹ is selected from hydrogen, fluoro, and chloro;
R²² is selected from hydrogen, C₁₋₄alkyl, deuterated C₁₋₄alkyl, and C₃₋₄ cycloalkyl, wherein said C₁₋₄alkyl may optionally be substituted one or more times with fluoro, hydroxy, C₁₋₄alkoxy, or C₃₋₄ cycloalkyl;
each R²³ is independently selected from hydrogen, halogen, and C₁₋₄alkyl; or
both R²³ together with the carbon they are attached to form a C₃₋₆ cycloalkyl or 3-6 membered heterocyclic ring, wherein the heterocyclic ring contains one to two heteroatoms selected from O;
or a pharmaceutically acceptable salt or stereoisomer thereof.

In one embodiment the disclosure provides a compound of formula (I′)

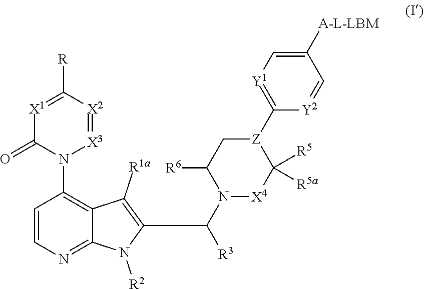

wherein:
X¹ is selected from N and CR¹¹; X² is selected from N and CR¹²; and X³ is selected from N and CR¹³, provided that only one of X¹, X² and X³ may be N;
X⁴ is CR⁴R⁴ or CO;
Y¹ is selected from N and CR⁷;
Y² is selected from N and CR⁸;
Z is selected from N and CR¹⁰;
R is NHR⁰;
R⁰ is selected from hydrogen, C₁₋₄alkyl, C₃₋₄ cycloalkyl, C₃₋₄ cycloalkyl-C₁₋₄alkyl, phenyl-C₁₋₄alkyl, halo-C₁₋₄alkyl, —(CH₂)ₙ—O—C₁₋₄alkyl, —(CH₂)ₙ—CO—R′, and —(CH₂)ₙ—CO₂R′, wherein said phenyl is optionally substituted one or more times with a substituent independently selected from halogen, C₁₋₄alkyl, halo-C₁₋₄alkyl, C₃₋₄cycloalkyl, hydroxy, C₁₋₄alkoxy, cyano, —NR′R″, —CONR′R″, and —CO₂R′, and wherein R′ and R″ are each independently selected from hydrogen and C₁₋₄alkyl, and n is 0, 1, or 2;
R^{1a} is hydrogen;
R² is selected from hydrogen, C₁₋₄alkyl, and deuterated C₁₋₄alkyl; wherein said C₁₋₄alkyl may optionally be substituted one or more times with halogen;
R³ is selected from hydrogen, C₁₋₄alkyl, and deuterated C₁₋₄alkyl;
R⁴ and R⁶ are independently selected from hydrogen and C₁₋₄alkyl, wherein said C₁₋₄alkyl may optionally be substituted with one or more halogens;
R⁵ is selected from hydrogen, halogen, C₁₋₄alkyl, and C₁₋₄alkoxy, wherein said C₁₋₄alkyl and C₁₋₄alkoxy may optionally be substituted with one or more halogen;
R¹⁰ is selected from hydrogen and fluoro;
or R⁵ and R¹⁰ together form a bond between the two carbons to which they are attached;
R^{5a} is hydrogen; or R⁵ and R^{5a} are both fluoro;
R⁷ and R⁸ are independently selected from hydrogen, halogen, C₁₋₄alkyl, C₃₋₄ cycloalkyl, and C₁₋₄alkoxy, wherein said C₁₋₄alkyl and C₁₋₄alkoxy may independently optionally be substituted with one or more halogen;
R¹¹ is selected from hydrogen, halogen, C₁₋₄alkyl, C₃₋₄cycloalkyl, C₁₋₄alkoxy, hydroxy, cyano, —NR′R″, —CONR′R″, —CO₂R′, —CO—(CH₂)ₘOH, wherein said C₁₋₄alkyl is optionally substituted one or more times with a substituent independently selected from halogen, hydroxy and C₁₋₄alkoxy, and wherein m is 1, 2 or 3 and wherein R′ and R″ are each independently selected from hydrogen and C₁₋₄alkyl; or
R¹¹ and R⁰ together with the atoms attached thereto may form a 5-6 membered heterocyclic or heteroaromatic ring containing one to two heteroatoms selected from N, wherein said 5-6 membered heterocyclic or heteroaromatic ring containing one to two heteroatoms selected from N is optionally substituted one or more times with a substituent independently selected from halogen, C₁₋₄alkyl, halo-C₁₋₄alkyl, C₃₋₄cycloalkyl, hydroxy, C₁₋₄alkoxy, cyano, —NR′R″, —CONR′R″ and —CO₂R′, wherein said C₁₋₄alkyl may optionally be substituted one or more times with a substituent independently selected from halogen and C₁₋₄alkoxy, and wherein R′ and R″ are each independently selected from hydrogen and C₁₋₄alkyl;
R¹² and R¹³ are independently selected from hydrogen, halogen, C₁₋₄alkyl, C₃₋₄ cycloalkyl, C₁₋₄alkoxy, hydroxy, cyano, —NR′R″, —CONR′R″, —CO₂R′, —CO—CO₂R′ and —CO—(CH₂)ₘOH, wherein said C₁₋₄alkyl is optionally substituted one or more times with a substituent independently selected from halogen, hydroxy, and C₁₋₄alkoxy, and wherein m is 1, 2 or 3 and wherein R′ and R″ are each independently selected from hydrogen and C₁₋₄alkyl;
A is CO;
L is selected from

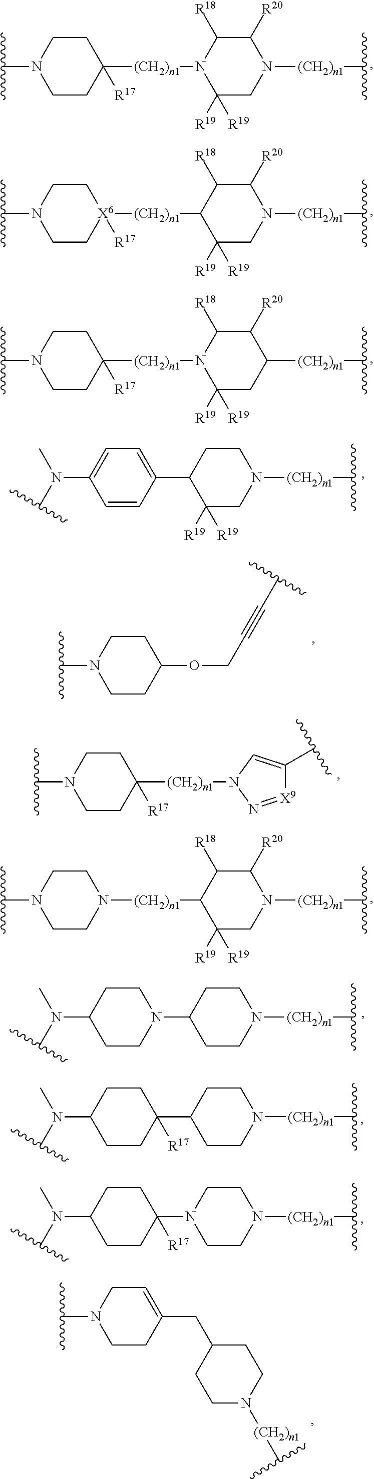

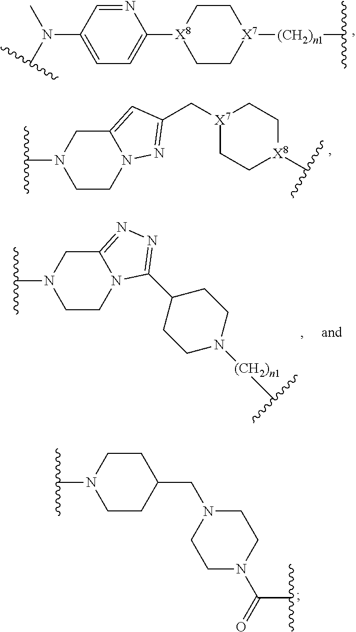

wherein n1 is selected from 0 and 1;
n2 is selected from 1 and 2;
R¹⁷ is selected from hydrogen and fluoro;
R¹⁸ is selected from hydrogen, fluoro, C₁₋₄alkyl wherein said C₁₋₄alkyl may optionally be substituted one or more times with fluoro or C₁₋₄alkoxy;
R¹⁹ is independently selected from hydrogen, C₁₋₄alkyl and fluoro;
R²⁰ is selected from hydrogen and C₁₋₄alkyl;
X⁶ is C;
X⁷ is CR²⁴ or N;
R²⁴ is selected from hydrogen and hydroxy;
X⁸ and X⁹ are independently CH or N; and
LBM is

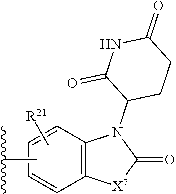

wherein X⁷ is O, NR²², or CR²³R²³;
R²¹ is selected from hydrogen, fluoro, and chloro;
R²² is selected from hydrogen, C₁₋₄alkyl, deuterated C₁₋₄alkyl, and C₃₋₄ cycloalkyl, wherein said C₁₋₄alkyl may optionally be substituted one or more times with fluoro, hydroxy, C₁₋₄ alkoxy, or C₃₋₄ cycloalkyl;
each R²³ is independently selected from hydrogen, halogen, and C₁₋₄alkyl; or
both R²³ together with the carbon they are attached to form a C₃₋₆ cycloalkyl or 3-6 membered heterocyclic ring, wherein the heterocyclic ring contains one to two heteroatoms selected from O;
or a pharmaceutically acceptable salt or stereoisomer thereof.

In one embodiment the disclosure provides a compound of formula (II) or (II′)

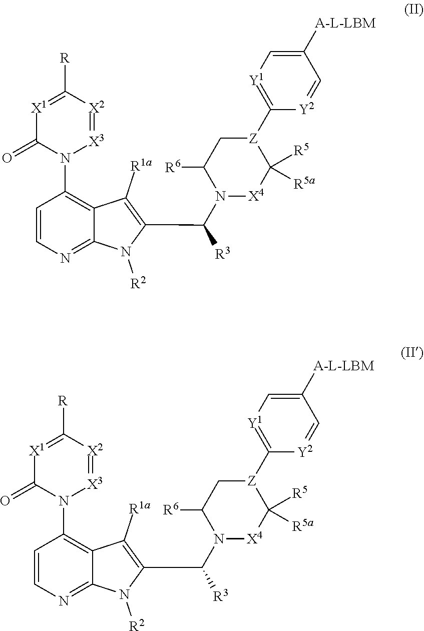

wherein X¹, X², X³, X⁴, Y¹, Y², Z, R, R^{1a}, R², R⁵, R^{5a}, R⁶, A, L, and LBM are as defined anywhere herein; and R³ is C₁₋₄alkyl;
or a pharmaceutically acceptable salt or stereoisomer thereof.

In some embodiments, X¹ is N, X² is CR¹², X³ is CR¹³, and X⁴ is CR¹⁴.

In some embodiments, X¹ is CR¹¹, X² is CR¹², X³ is CR¹³, and X⁴ is CR¹⁴.

In one embodiment the disclosure provides a compound of formula (III)

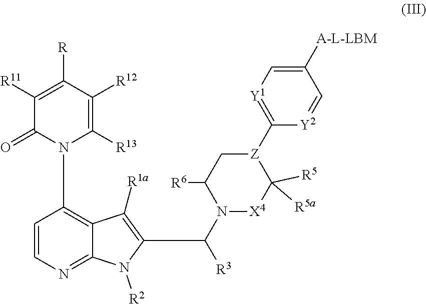

wherein Y¹, Y², X⁴, Z, R, R^{1a}, R², R³, R⁵, R^{5a}, R⁶, R¹¹, R¹², R¹³, A, L, and LBM are as defined anywhere herein; or a pharmaceutically acceptable salt or stereoisomer thereof.

In one embodiment the disclosure provides a compound of formula (IV)

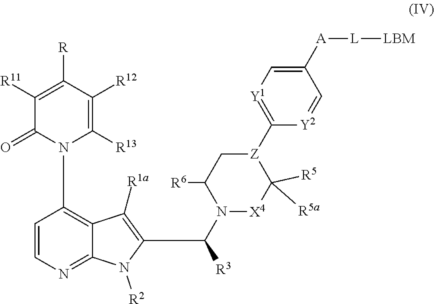

wherein Y¹, Y², X⁴, Z, R, R^{1a}, R², R⁵, R^{5a}, R⁶, R¹¹, R¹², R¹³, A, L, and LBM are as defined anywhere herein; and R³ is C₁₋₄alkyl; or a pharmaceutically acceptable salt or stereoisomer thereof.

In one embodiment the disclosure provides a compound of formula (IV′)

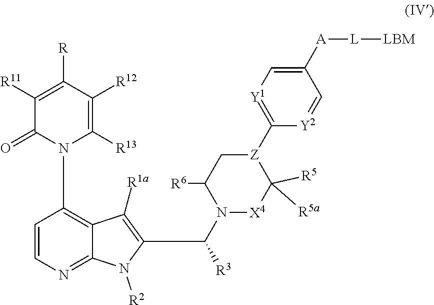

wherein Y¹, Y², X⁴, Z, R, R^{1a}, R², R⁵, R^{5a}, R⁶, R¹¹, R¹², R¹³, A, L, and LBM are as defined anywhere herein; and R³ is C₁₋₄alkyl; or a pharmaceutically acceptable salt or stereoisomer thereof.

In some embodiments, each of R¹¹, R¹², R¹³, and R¹⁴, when present, is independently selected from hydrogen, halogen, and C₁₋₄alkoxy.

In some embodiments, R¹² and R¹³ are both hydrogen.

In some embodiments, R¹¹, R¹², and R¹³ are all hydrogen.

In some embodiments, X¹ is CR¹¹; X² is CR¹²; X³ is CR¹³; X⁴ is CR¹⁴; and R¹¹ and R⁰ together with the atoms attached thereto may form a 5-6 membered heterocyclic or heteroaromatic ring containing one to two heteroatoms selected from N, wherein said 5-6 membered heterocyclic or heteroaromatic ring containing one to two heteroatoms selected from N is optionally substituted one or more times with a substituent independently selected from halogen, C₁₋₄alkyl, halo-C₁₋₄alkyl, C₃₋₄ cycloalkyl, hydroxy, C₁₋₄alkoxy, cyano, —NR′R″, —CONR′R″ and —CO₂R′, wherein said C₁₋₄alkyl may optionally be substituted one or more times with a substituent independently selected from halogen and C₁₋₄alkoxy, and wherein R′ and R″ are each independently selected from hydrogen and C₁₋₄alkyl. In some embodiments, the 5-6 membered heteroaromatic ring containing one or two N atoms is a pyrazole ring.

In some embodiments, the 5-6 membered heterocyclic ring containing one or two N atoms is a pyrrolidine ring.

In some embodiments, R is NHR⁰ and R⁰ is hydrogen.

In some embodiments, R is NHR⁰ and R⁰ is selected from C₁₋₄alkyl, C₃₋₄cycloalkyl, C₃₋₄ cycloalkyl-C₁₋₄alkyl, phenyl-C₁₋₄alkyl, halo-C₁₋₄alkyl, —(CH₂)ₙ—O—C₁₋₄alkyl, —(CH₂)ₙ—CO—R′, and —(CH₂)ₙ—CO₂R′, wherein said phenyl is optionally substituted one or more times with a substituent independently selected from halogen, C₁₋₄alkyl, halo-C₁₋₄alkyl, C₃₋₄cycloalkyl, hydroxy, C₁₋₄alkoxy, cyano, —NR′R″, —CONR′R″, and —CO₂R′, and wherein R′ and R″ are each independently selected from hydrogen and C₁₋₄ alkyl, and n is 0, 1, or 2;

In some embodiments, R is NHR⁰ and R⁰ is selected from hydrogen and C₁₋₄alkyl.

In some embodiments, R is NHR⁰ and R⁰ is C₁₋₄alkyl (e.g., methyl).

In one embodiment the disclosure provides a compound of formula (VII)

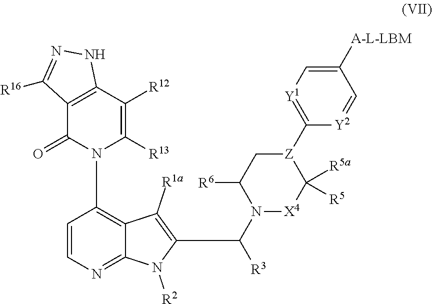

wherein Y¹, Y², X⁴, Z, R^{1a}, R², R³, R⁵, R^{5a}, R⁶, R¹², R¹³, A, L and LBM are as defined anywhere herein, R¹⁶ is selected from hydrogen and C₁₋₄alkyl, wherein said C₁₋₄alkyl may optionally be substituted one or more times with a substituent independently selected from halogen.

In one embodiment the disclosure provides a compound of the formula (VI II)

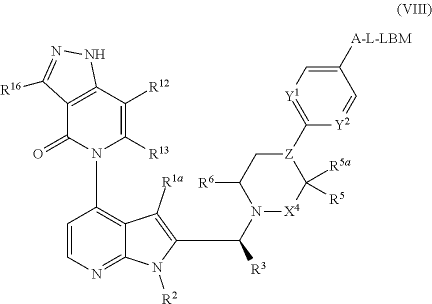

wherein Y¹, Y², X⁴, Z, R^{1a}, R², R³, R⁵, R^{5a}, R⁶, R¹², R¹³, A, L and LBM are as defined anywhere herein, and R¹⁶ is selected from hydrogen and C₁₋₄alkyl, wherein said C₁₋₄alkyl may optionally be substituted one or more times with a substituent independently selected from halogen.

In one embodiment the disclosure provides a compound of the formula (VIII′)

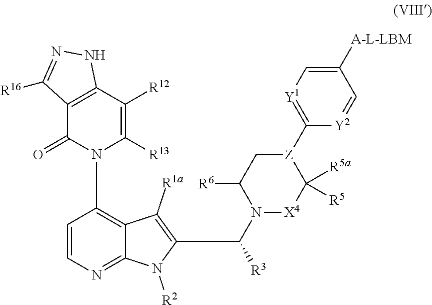

wherein Y¹, Y², X⁴, Z, R^{1a}, R², R³, R⁵, R^{5a}, R⁶, R¹², R¹³, A, L and LBM are as defined anywhere herein, and R¹⁶ is selected from hydrogen and C₁₋₄alkyl, wherein said C₁₋₄alkyl may optionally be substituted one or more times with a substituent independently selected from halogen.

In some embodiments, R¹⁶ is hydrogen.

In one embodiment the disclosure provides a compound of the formula (IX):

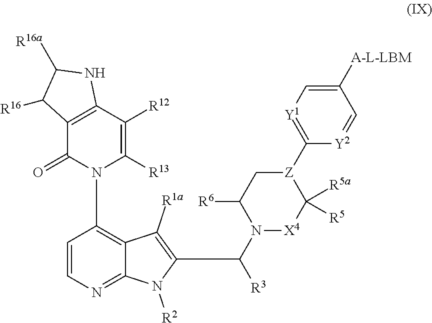

wherein R¹⁶ and R^{16a} are selected from hydrogen and C₁₋₄alkyl, wherein said C₁₋₄alkyl may optionally be substituted one or more times with a substituent independently selected from halogen; or a pharmaceutically acceptable salt or stereoisomer thereof.

In one embodiment the disclosure provides a compound of the formula (IXa) or (IXb):

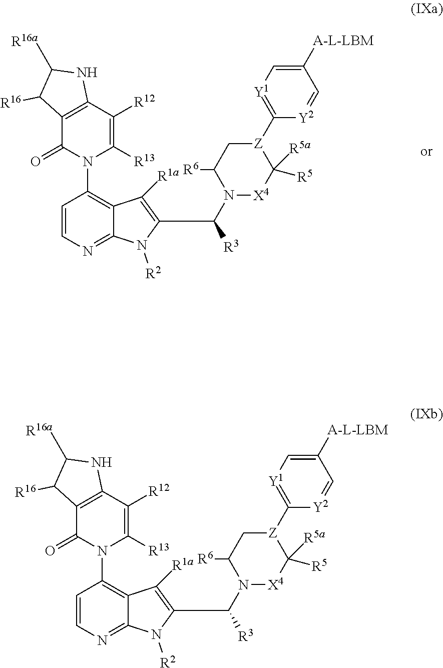

wherein R¹⁶ and R^{16a} are selected from hydrogen and C₁₋₄alkyl, wherein said C₁₋₄alkyl may optionally be substituted one or more times with a substituent independently selected from halogen; or a pharmaceutically acceptable salt or stereoisomer thereof.

In some embodiments, R¹⁶ and R^{16a} are both hydrogen.

In some embodiments, Z is N.

In some embodiments, Z is CR¹⁰ and R⁵ and R¹⁰ together form a bond between the two carbons to which they are attached.

In some embodiments, Z is CR¹⁰ and R¹⁰ is hydrogen.

In some embodiments, R^{5a} is hydrogen.

In some embodiments, X⁴ is CR⁴ and R⁴ is selected from hydrogen and C₁₋₄alkyl.

In some embodiments, the

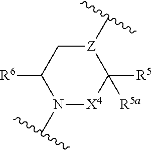

moiety is

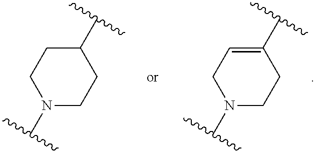 [SMILES: *C1CCN(*)CC1 ; *C1=CCN(*)CC1]

In some embodiments, Y¹ is N and Y² is CR⁸.

In some embodiments, Y¹ is N and Y² is N.

In some embodiments, Y¹ is CR⁷ and Y² is CR⁸.

In some embodiments, the disclosure provides a compound as described herein, wherein
(a) both of R⁷ and R⁸ is C₁₋₄alkyl;
(b) both of R⁷ and R⁸ is halogen;
(c) one of R⁷ and R⁸ is C₁₋₄alkyl and the other is halogen;
(d) one of R⁷ and R⁸ is C₁₋₄alkyl and the other is hydrogen; or
(e) one of R⁷ and R⁸ is halogen and the other is hydrogen.

In some embodiments, the disclosure provides a compound as described herein, wherein
(a) both of R⁷ and R⁸ is C₁₋₄alkyl;
(b) both of R⁷ and R⁸ is halogen;
(c) one of R⁷ and R⁸ is C₁₋₄alkyl and the other is halogen;
(d) one of R⁷ and R⁸ is C₁₋₄alkyl and the other is hydrogen; (e) one of R⁷ and R⁸ is halogen and the other is hydrogen; and
(f) both of R⁷ and R⁸ are hydrogen;

In some embodiments, both of R⁷ and R⁸ are halogen; or one of R⁷ and R⁸ is C₁₋₄alkyl and the other is halogen; or both of R⁷ and R⁸ are hydrogen. In some embodiments, both of R⁷ and R⁸ are halogen, or one of R⁷ and R⁸ is C₁₋₄alkyl and the other is halogen.

In some embodiments, R⁷ is halogen and R⁸ is methyl.

In some embodiments, the halogen in b) c) and e) is fluoro.

In some embodiments, the disclosure provides a compound as described herein, wherein R² is methyl.

In some embodiments, the disclosure provides a compound as described herein, wherein R³ is hydrogen or C₁₋₄alkyl.

In some embodiments, the disclosure provides a compound as described herein, wherein R³ is methyl.

### Precursor

In some embodiments, compounds of formula (I) are prepared from precursors in the following table, and subsequently derivatized at the appropriate position to achieve compounds of formula (I) (i.e. wherein R⁹ is -A-L-LBM).

| No. | Structure |
|---|---|
| 1ab1 | 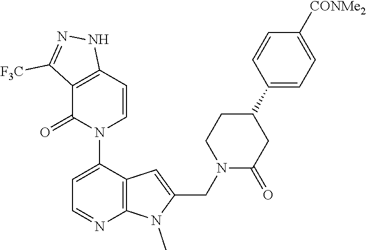 |
| 1ab2 | 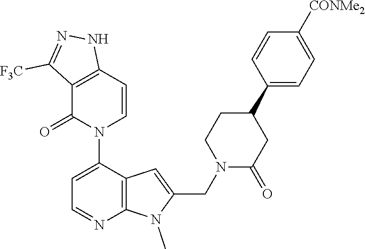 |
| 1ab3 | 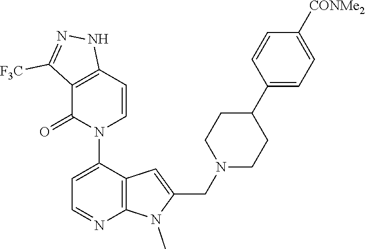 |
| 1ab4 | 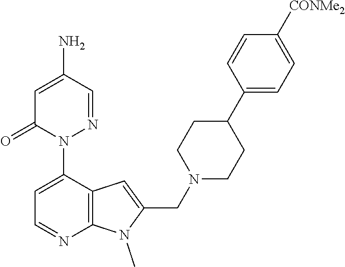 |
| 1ab5 | 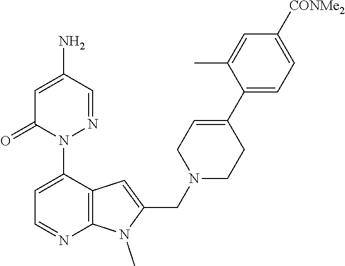 |
| 1ab6 | 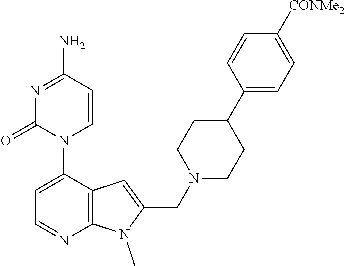 |
| 1ac1 | 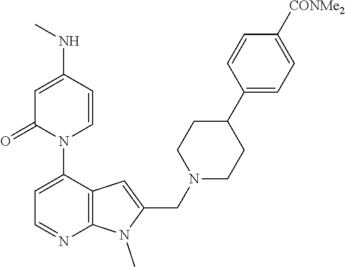 |
| 1ac2 | 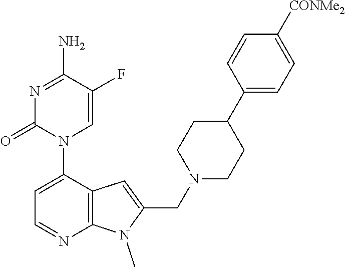 |
| 1ac3 | 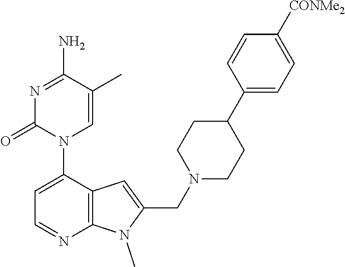 |
| 1ad5 | 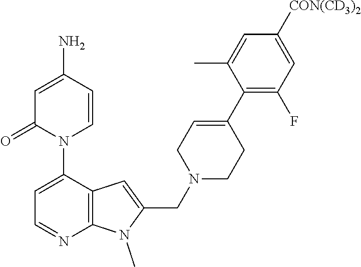 |
| 1ad6 | 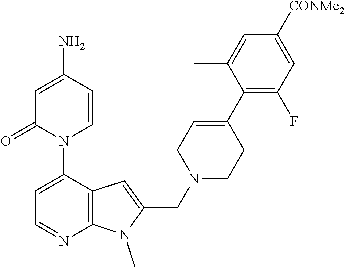 |
| 1ad7 | 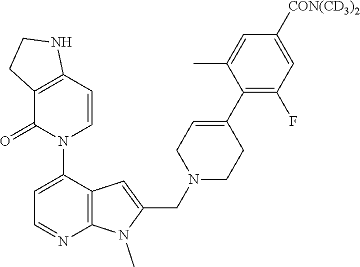 |
| 1ad8 | 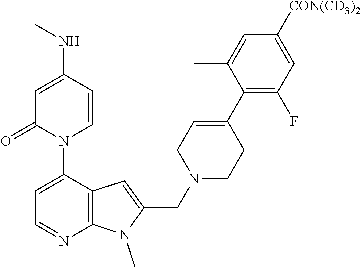 |
| 1ad9 | 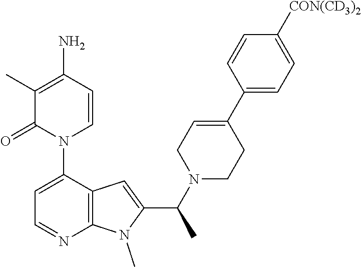 |
| 1ad10 | 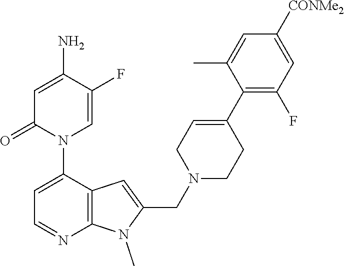 |
| 1ad11 | 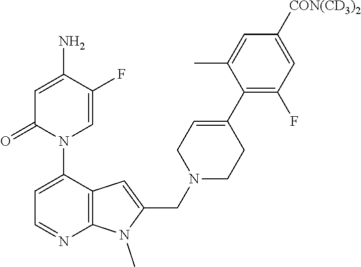 |
| 1af9 | 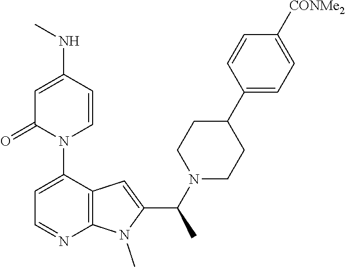 |
| 1af10 | 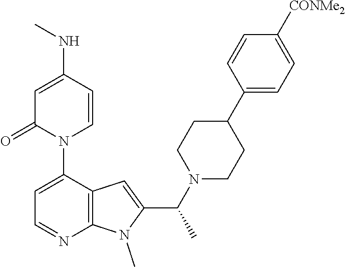 |
| 1af61 | 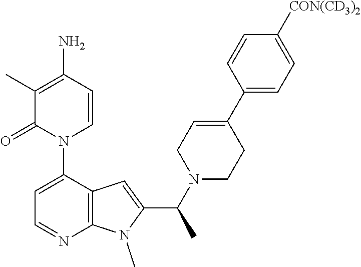 |
| 1 af62 | 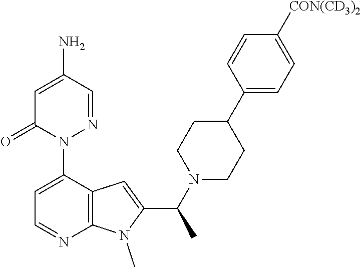 |
| 1af63 | 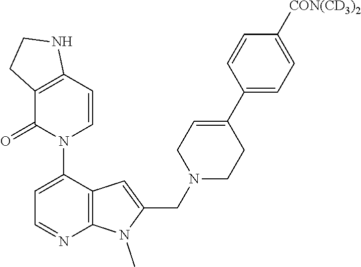 |
| 1af65 | 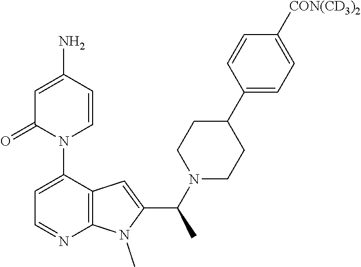 |
| 1af82 | 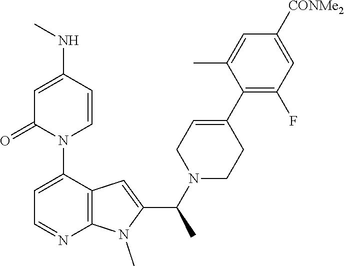 |
| 1af83 | 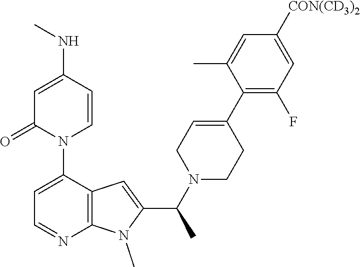 |
| 1d1 | 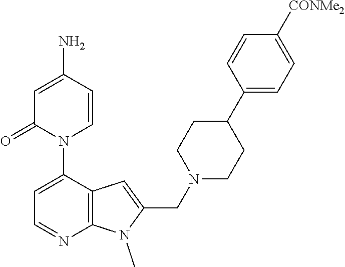 |
| 1d2 | 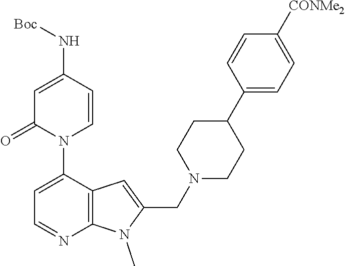 |
| 1d3 | 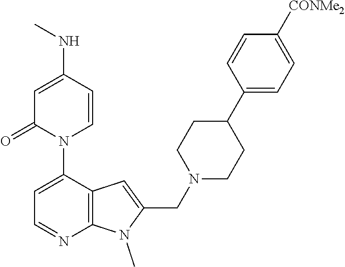 |
| 1d4 | 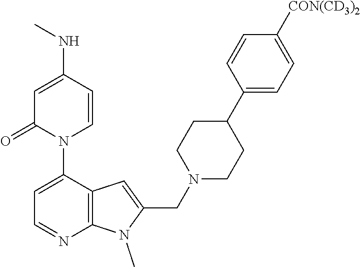 |
| 1d5 | 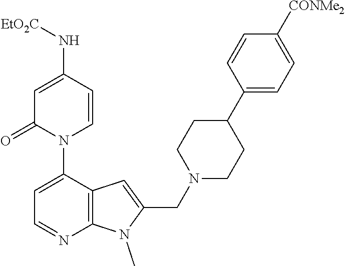 |
| 1d6 | 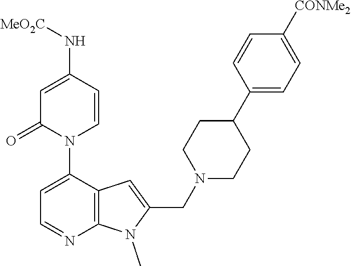 |
| 1d7 | 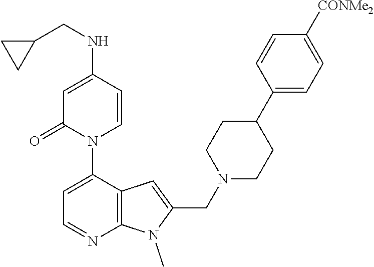 |
| 1d9 | 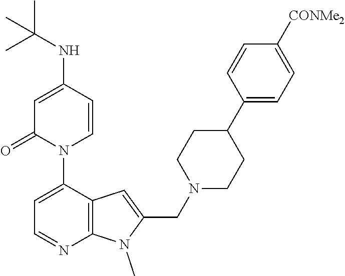 |
| 1d10 | 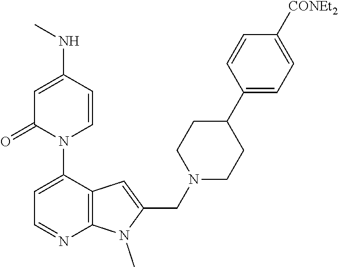 |
| 1d11 | 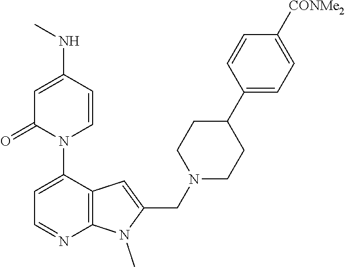 |
| 1d12 | 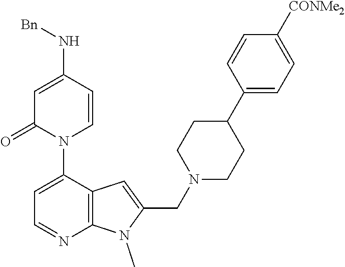 |
| 1d13 | 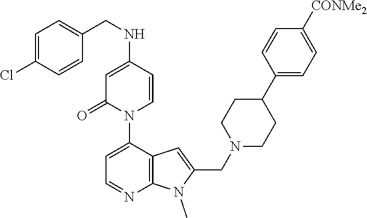 |
| 1d14 | 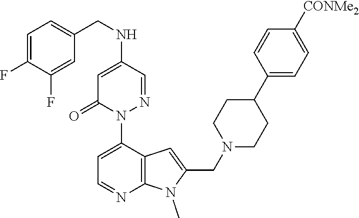 |
| 1d15 | 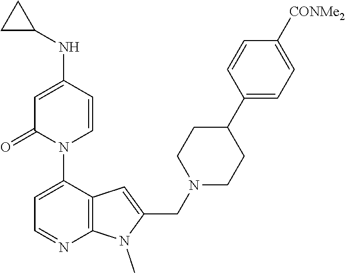 |
| 1d17 | 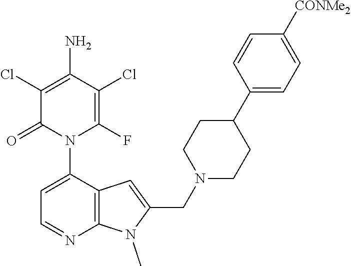 |
| 1d18 | 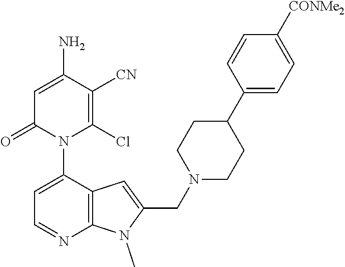 |
| 1d19 | 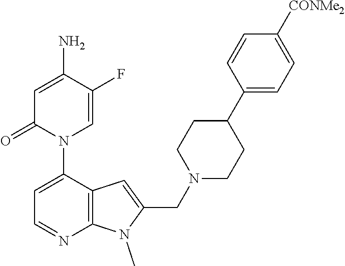 |
| 1d20 | 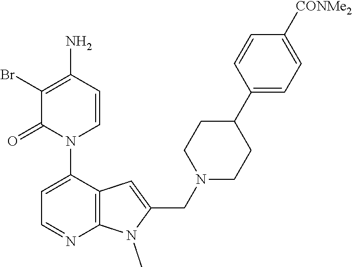 |
| 1d21 | 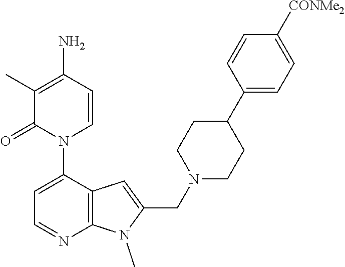 |
| 1d22 | 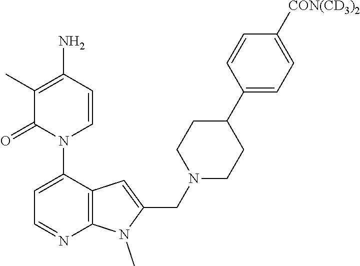 |
| 1d23 | 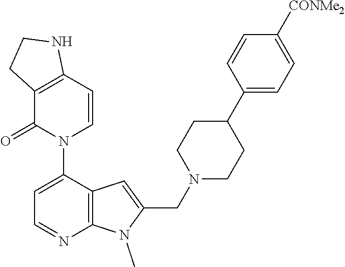 |
| 1d24 | 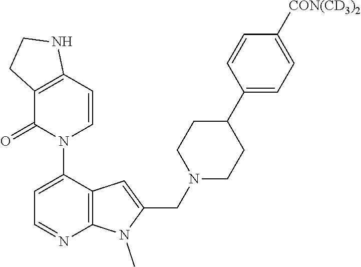 |
| 1d25 | 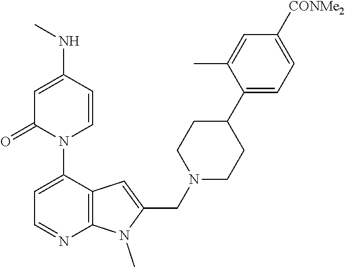 |
| 1d26 | 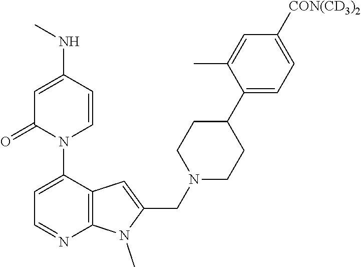 |
| 1d27 | 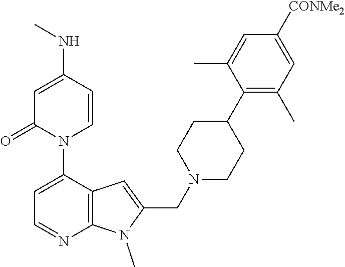 |
| 1d28 | 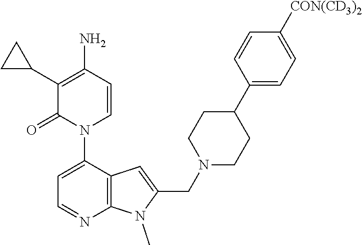 |
| 1d29 | 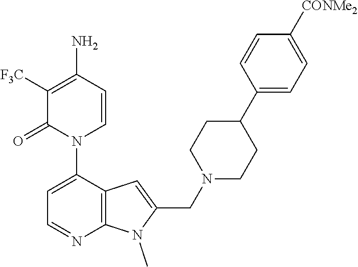 |
| 1d30 | 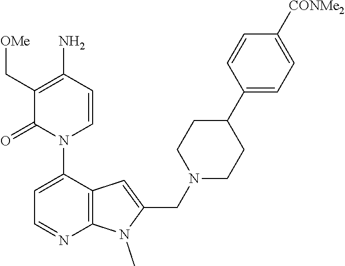 |
| 1d31 | 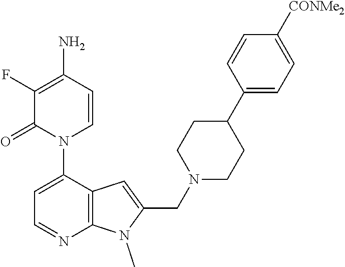 |
| 1d32 | 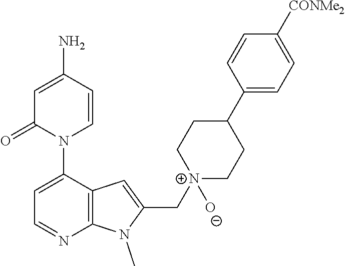 |
| 1d33 | 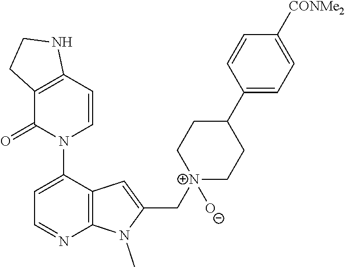 |
| 1d34 | 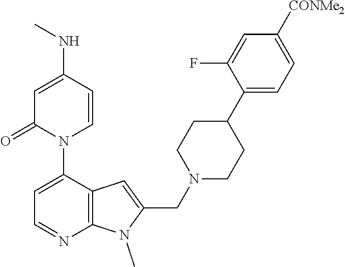 |
| 1d35 | 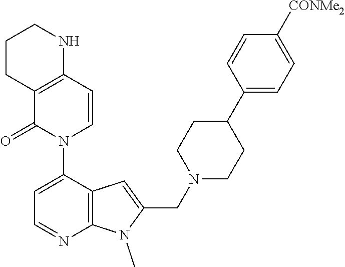 |
| 1d36 | 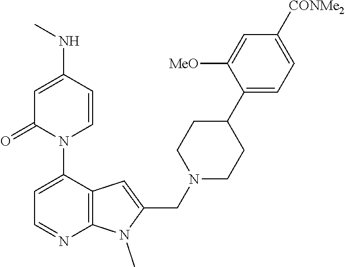 |
| 1d37 | 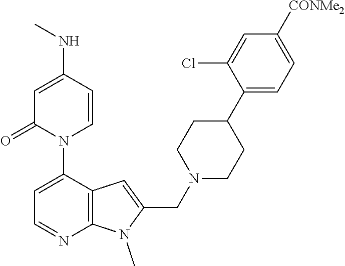 |
| 1d38 | 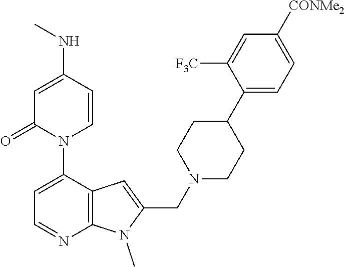 |
| 1d39 | 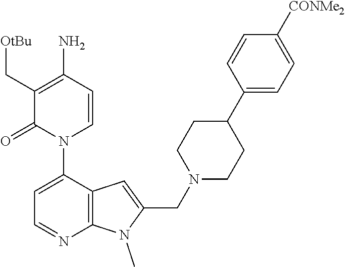 |
| 1d40 | 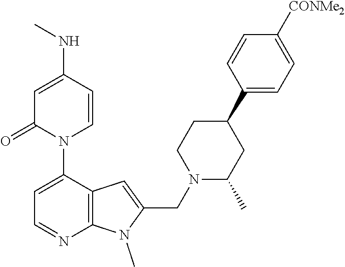 |
| 1d41 | 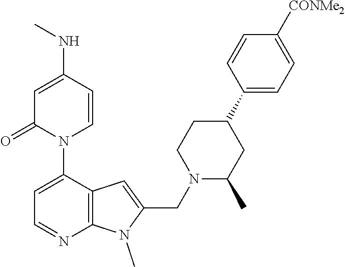 |
| 1d42 | 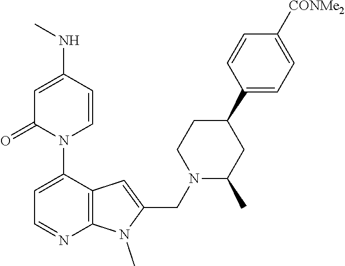 |
| 1d43 | 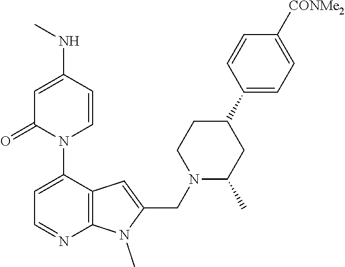 |
| 1d44 | 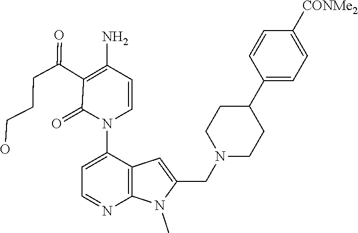 |
| 1f1 | 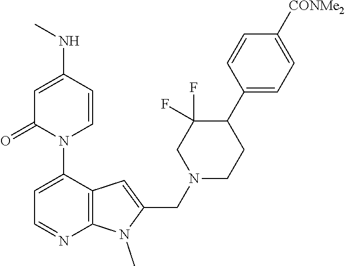 |
| 1f2 | 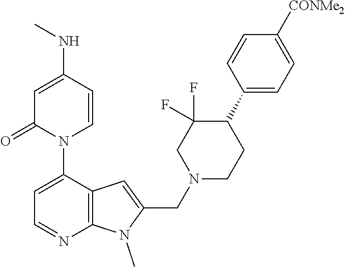 |
| 1f3 | 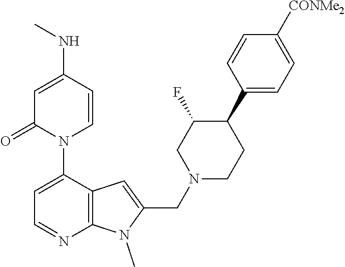 |
| 1f4 | 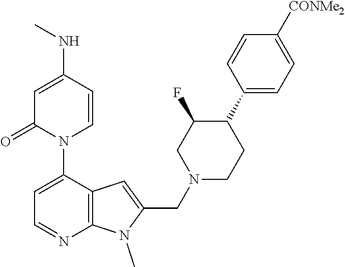 |
| 1f5 | 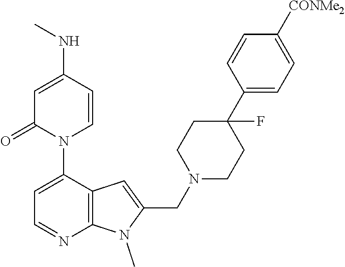 |
| 1f6 | 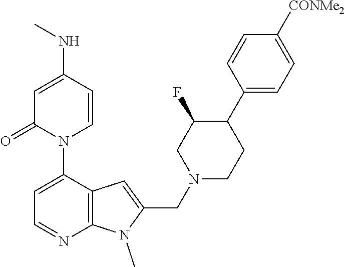 |
| 1f7 | 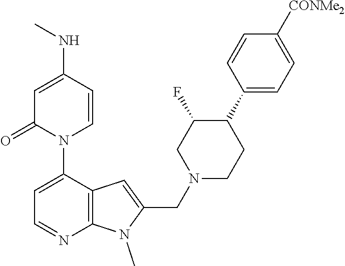 |
| 1f8 | 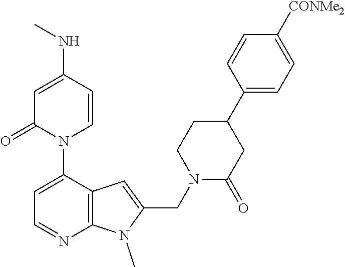 |
| 1j27 | 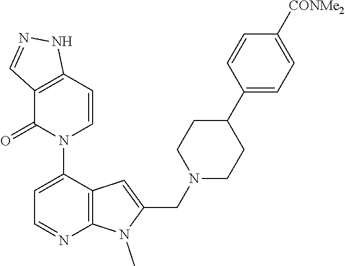 |
| 1m1 | 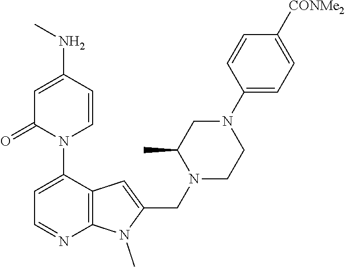 |
| 1m2 | 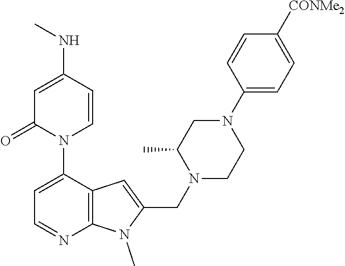 |
| 1m3 | 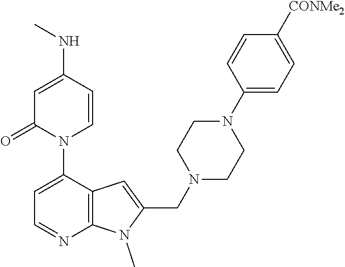 |
| 1m6 | 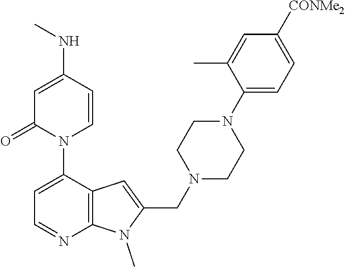 |
| 1m7 | 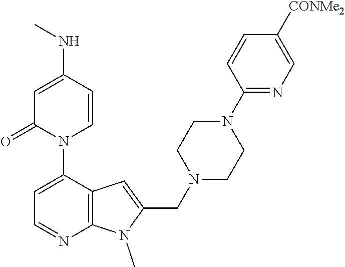 |
| 1m8 | 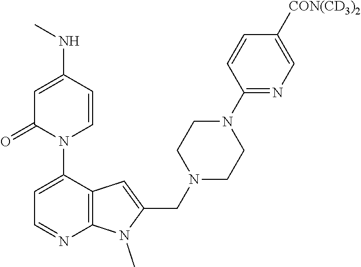 |
| 1m9 | 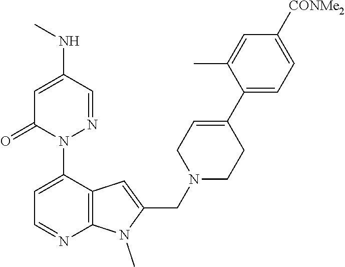 |
| 1m10 | 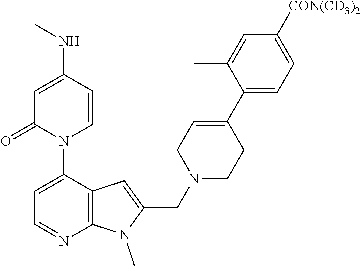 |
| 1m11 | 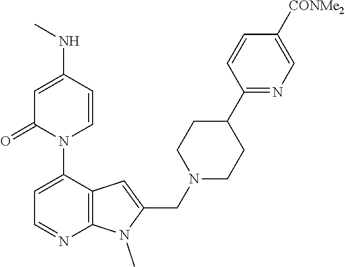 |
| 1m12 | 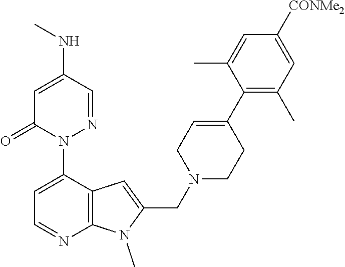 |
| 1m13 | 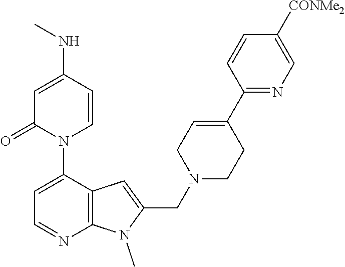 |
| 1m14 | 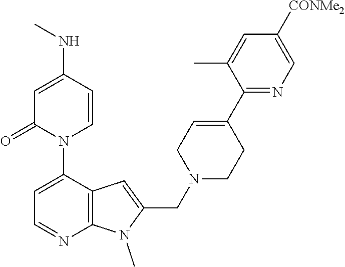 |
| 1m15 | 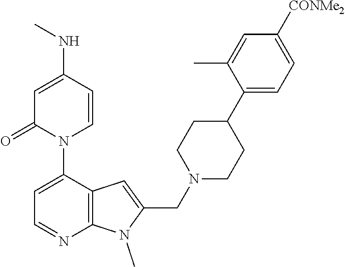 |
| 1m18 | 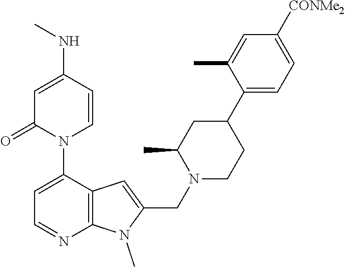 |
| 1m19 | 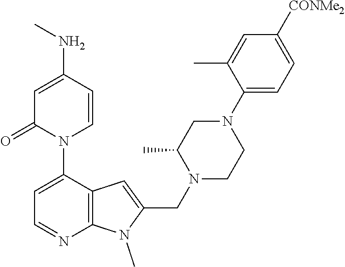 |
| 1m35 | 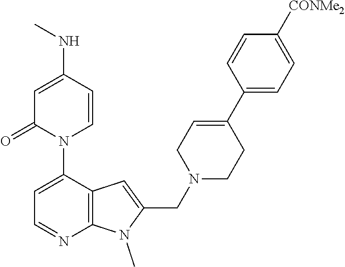 |
| 1m54 | 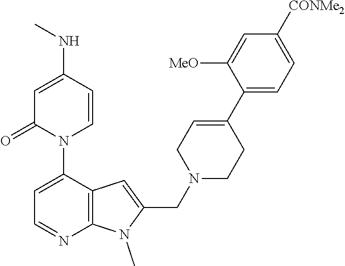 |
| 1m56 | 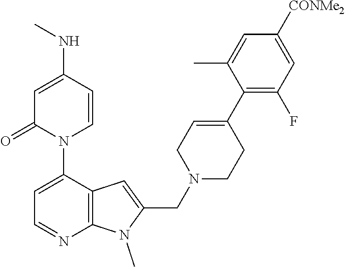 |
| 1m57 | 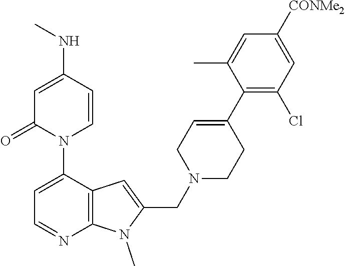 |
| 1m59 | 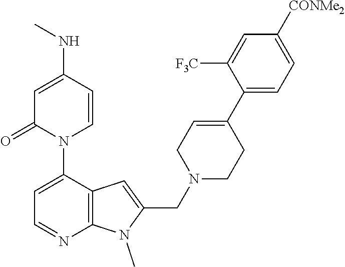 |
| 1m64 | 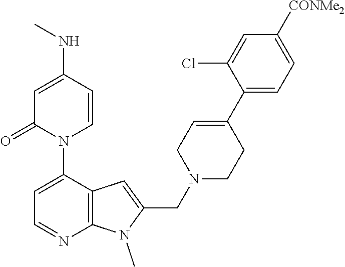 |
| 1m67 | 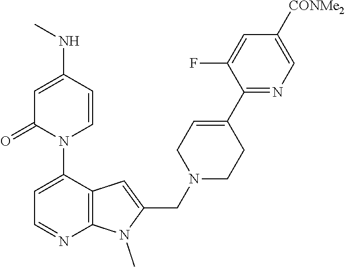 |
| 1m69 | 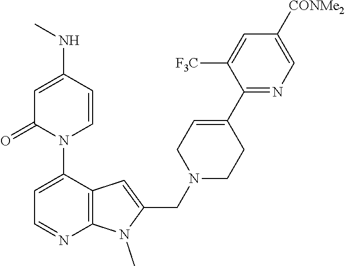 |
| 1m70 | 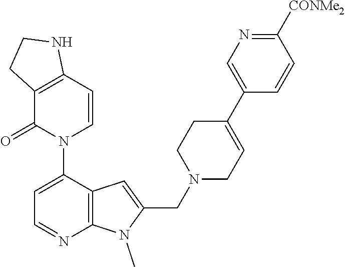 |
| 1m71 | 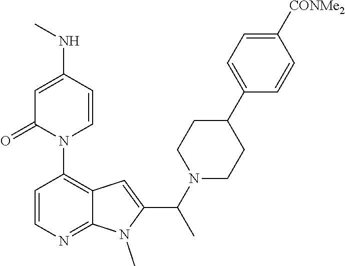 |
| 1m72 | 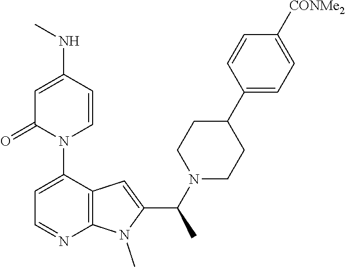 |
| 1m73 | 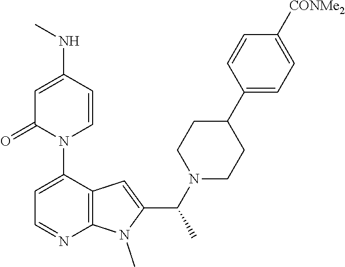 |
| 1m74 | 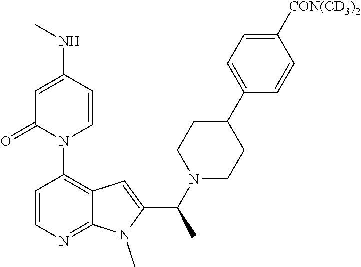 |
| 1m75 | 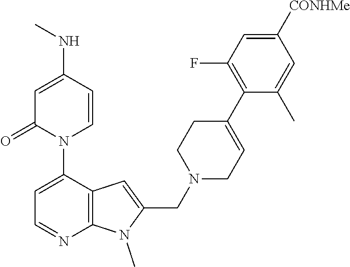 |
| 1m76 | 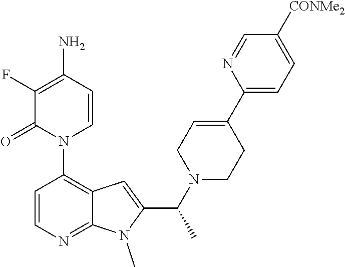 |
| 1m77 | 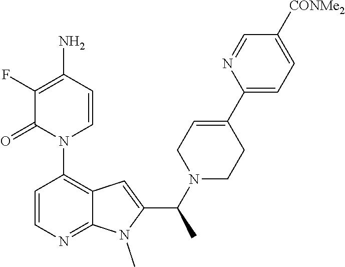 |
| 1m78 | 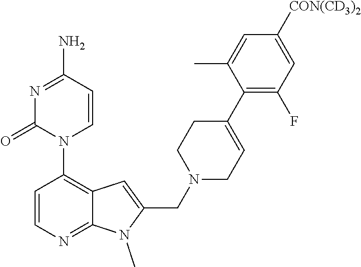 |
| 1m79 | 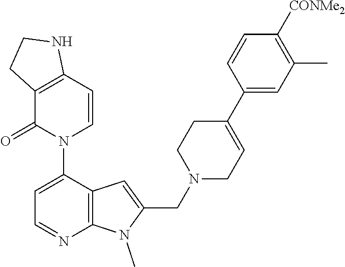 |
| 1m80 | 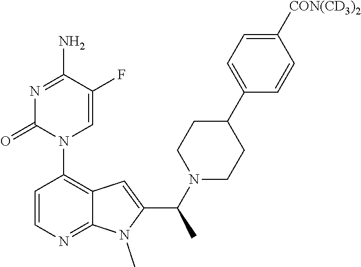 |
| 1m81 | 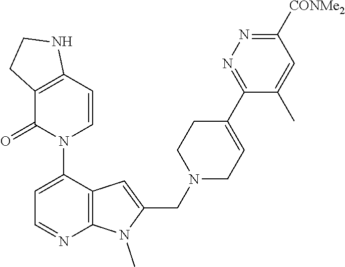 |
| 1m82 | 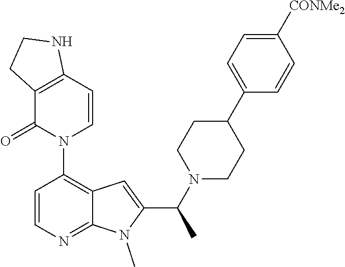 |
| 1m83 | 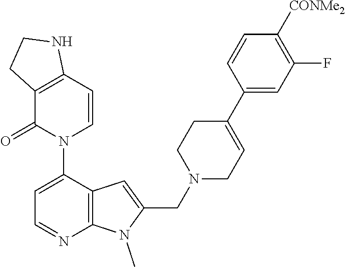 |
| 1m84 | 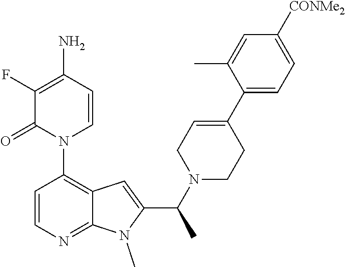 |
| 1m85 | 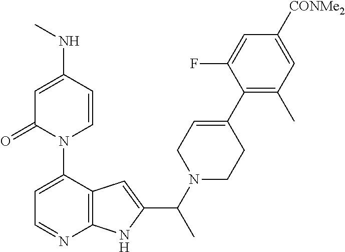 |
| 1m86 | 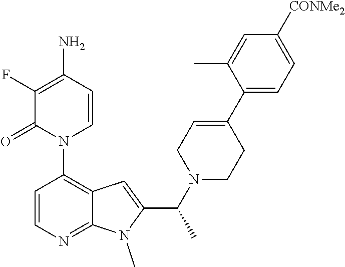 |
| 1m87 | 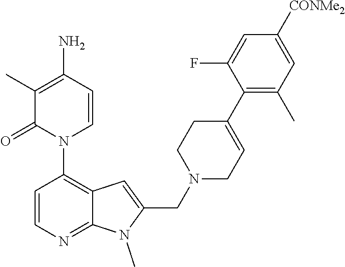 |
| 1m88 | 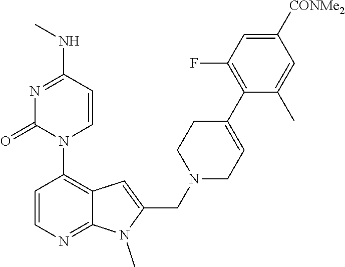 |
| 1m89 | 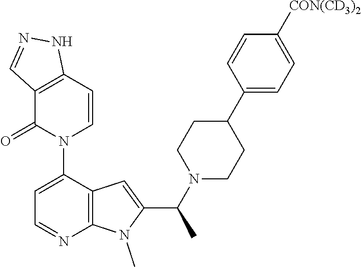 |
| 1m90 | 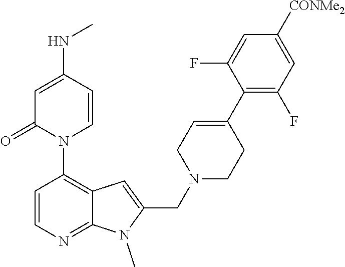 |
| 1m91 | 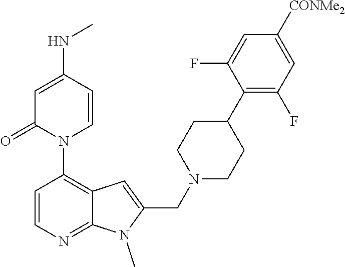 |
| 1m93 | 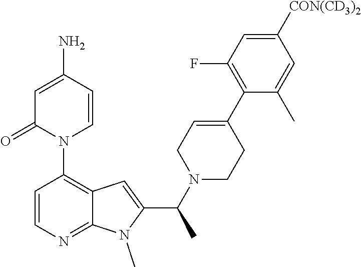 |
| 1m94 | 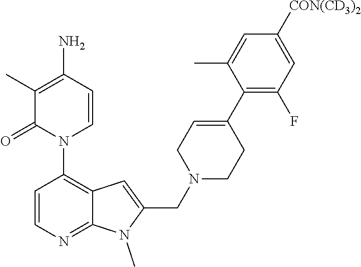 |
| 1m95 | 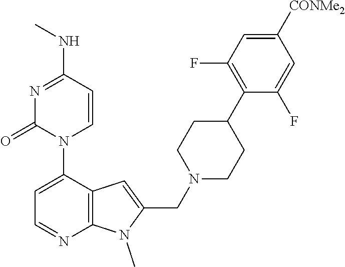 |
| 1m96 | 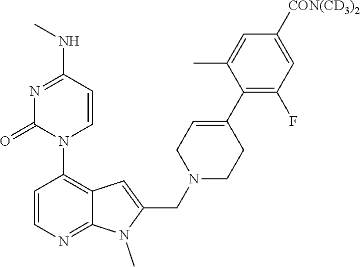 |
| 1m97 | 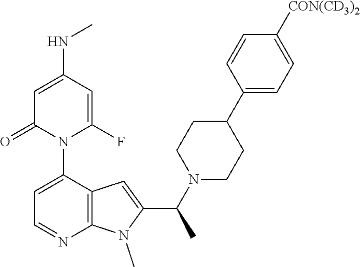 |
| 1m98 | 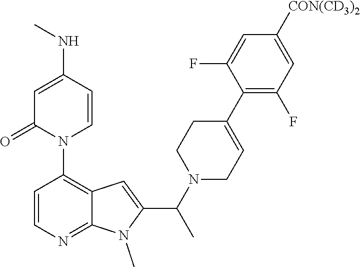 |
| 1m99 | 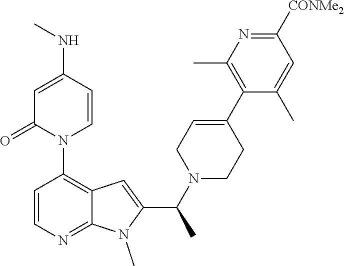 |
| 1m100 | 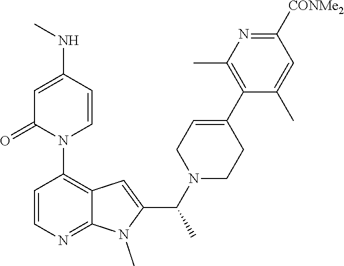 |
| 1m101 | 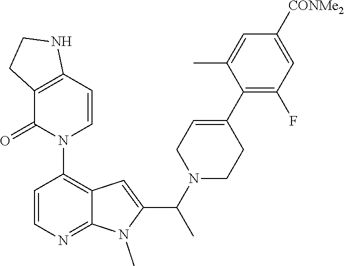 |
| 1m102 | 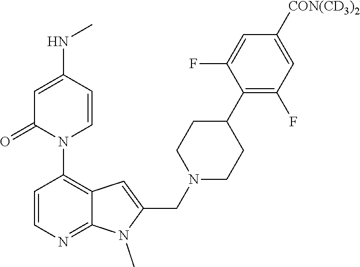 |
| 1m103 | 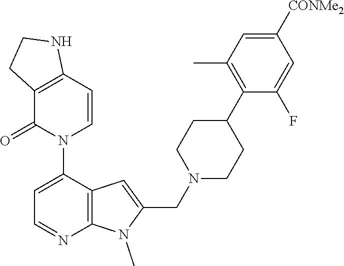 |
| 1m104 | 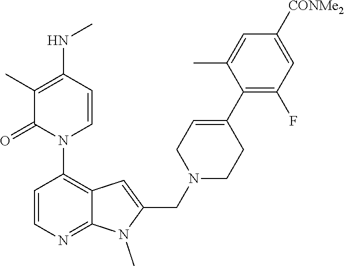 |
| 1m105 | 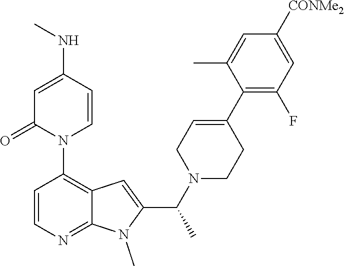 |
| 1m106 | 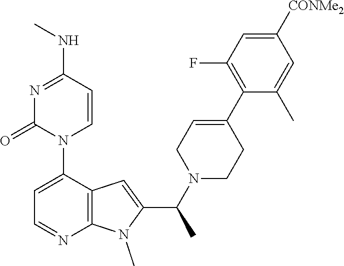 |
| 1m107 | 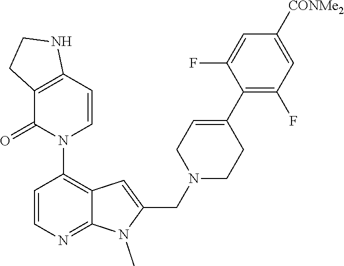 |
| 1m108 | 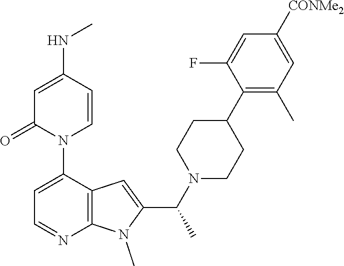 |
| 1m109 | 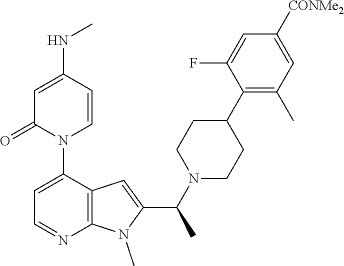 |
| 1m110 | 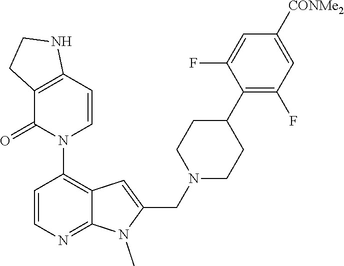 |
| 1m111 | 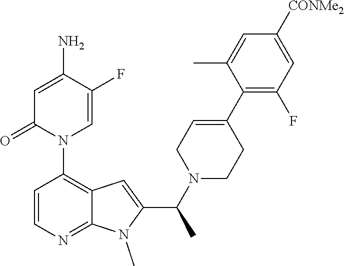 |
| 1m112 | 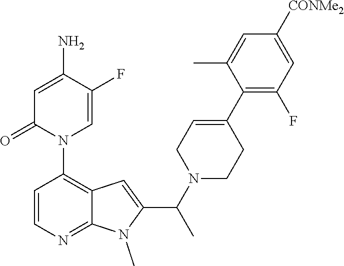 |
| 1m113 | 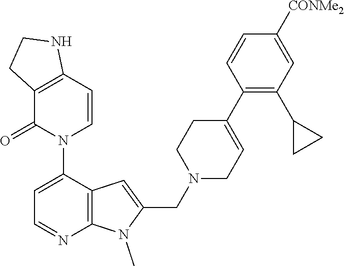 |
| 1m114 | 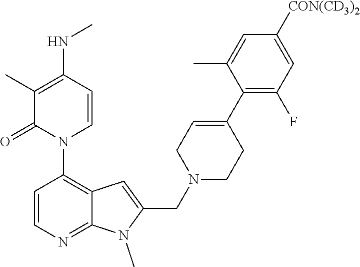 |
| 1m115 | 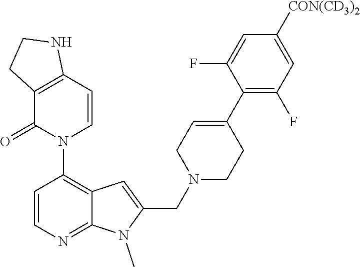 |
| 1m116 | 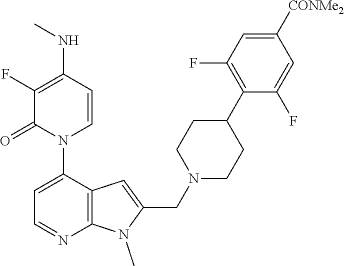 |
| 1m117 | 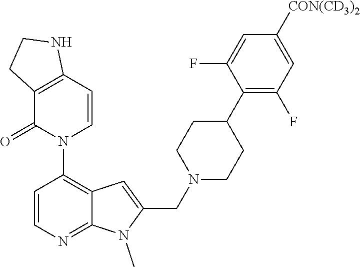 |
| 1m118 | 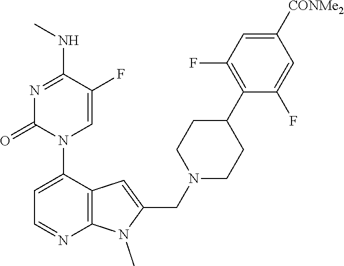 |
| 1m119 | 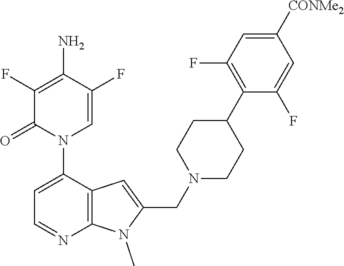 |
| 1m120 | 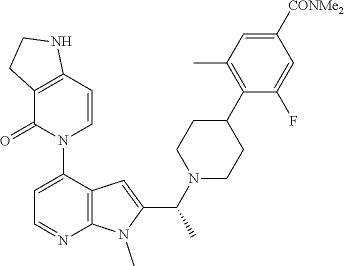 |
| 1m121 | 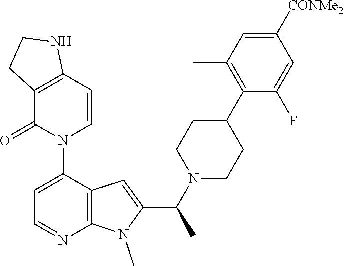 |
| 1m122 | 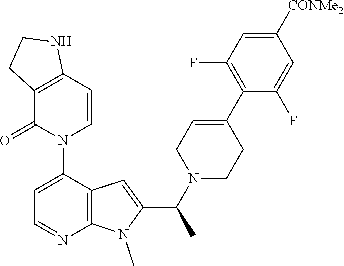 |
| 1m123 | 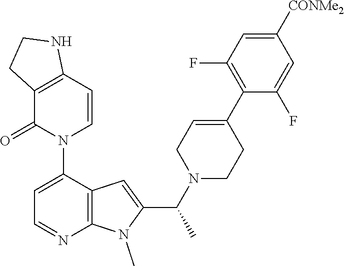 |
| 1m124 | 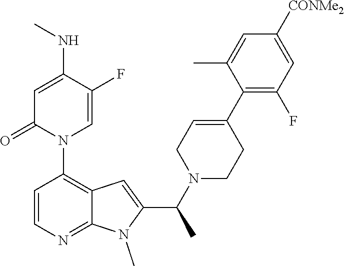 |
| 1m125 | 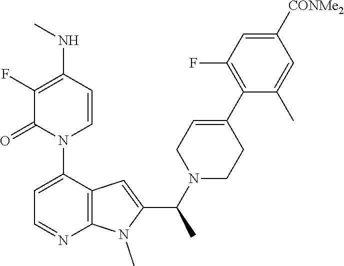 |
| 1m126 | 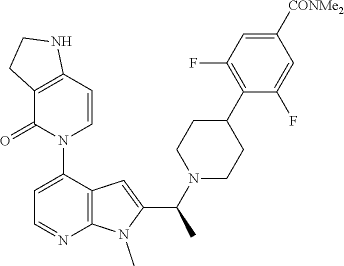 |
| 1m127 | 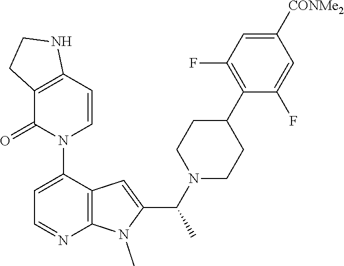 |
| 1m128 | 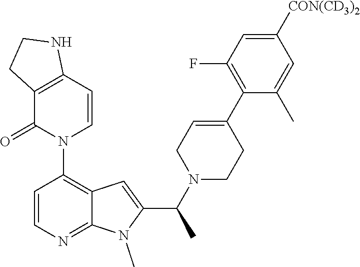 |
| 1m129 | 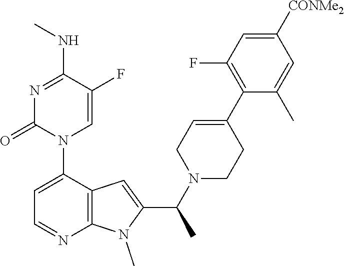 |
| 1m130 | 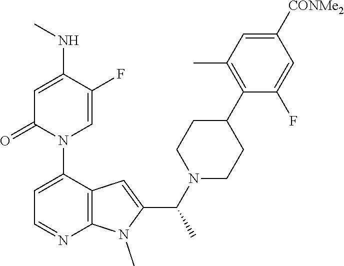 |
| 1m131 | 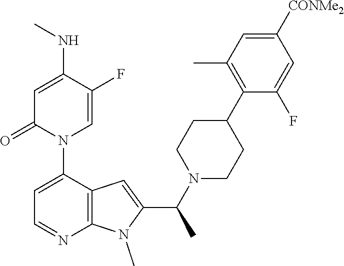 |
| 1m132 | 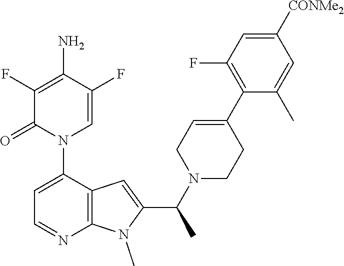 |
| 1m133 | 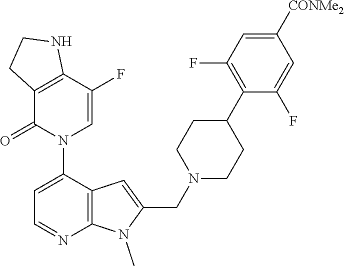 |
| 1m134 | 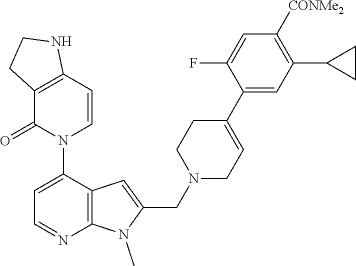 |
| 1m135 | 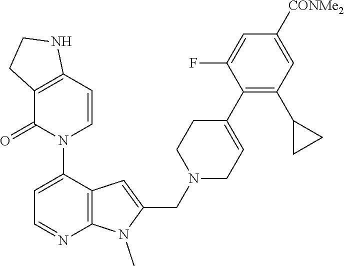 |
| 1m136 | 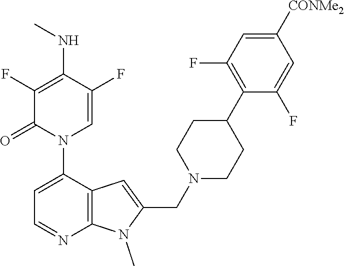 |
| 1m137 | 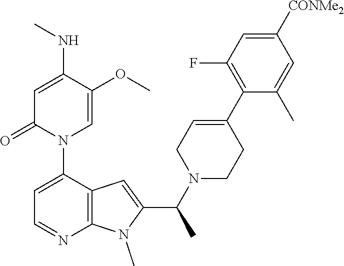 |
| 1m138 | 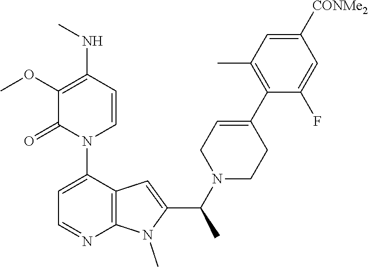 |
| 1m139 | 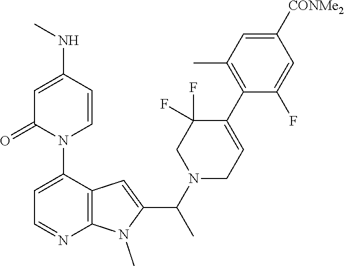 |
| 1m140 | 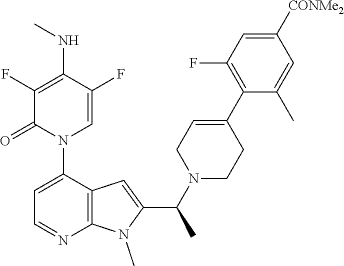 |
| 1m141 | 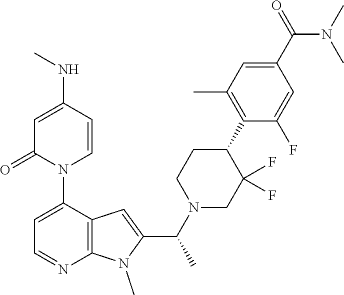 [SMILES: CNc1ccn(-c2ccnc3c2cc([C@@H](C)N2CC[C@H](c4c(C)cc(C(=O)N(C)C)cc4F)C(F)(F)C2)n3C)c(=O)c1] |
| 1m142 | 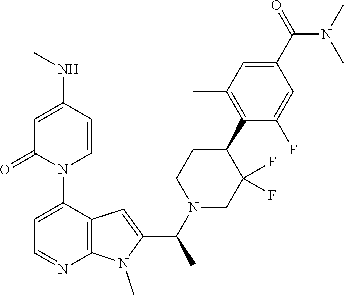 [SMILES: CNc1ccn(-c2ccnc3c2cc([C@H](C)N2CC[C@@H](c4c(C)cc(C(=O)N(C)C)cc4F)C(F)(F)C2)n3C)c(=O)c1] |
| 1m143 | 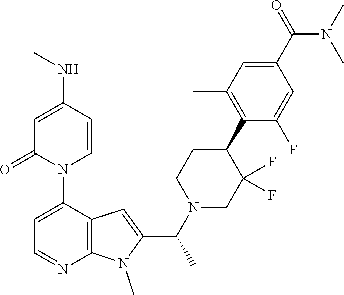 [SMILES: CNc1ccn(-c2ccnc3c2cc([C@@H](C)N2CC[C@@H](c4c(C)cc(C(=O)N(C)C)cc4F)C(F)(F)C2)n3C)c(=O)c1] |
| 1m144 | 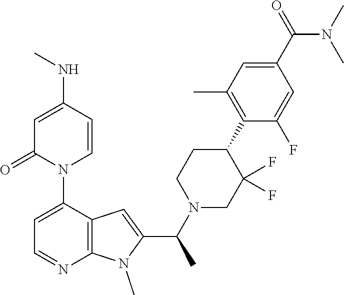 [SMILES: CNc1ccn(-c2ccnc3c2cc([C@H](C)N2CC[C@H](c4c(C)cc(C(=O)N(C)C)cc4F)C(F)(F)C2)n3C)c(=O)c1] |
| 1m146 | 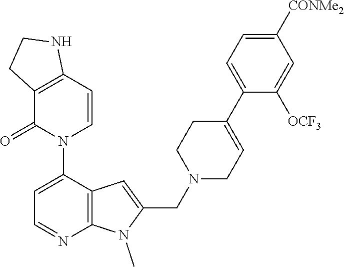 |
| 1m147 | 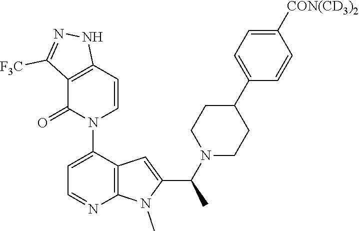 |

### Moiety A and Moiety L

In some embodiments, A is CO.

In some embodiments, L is selected from

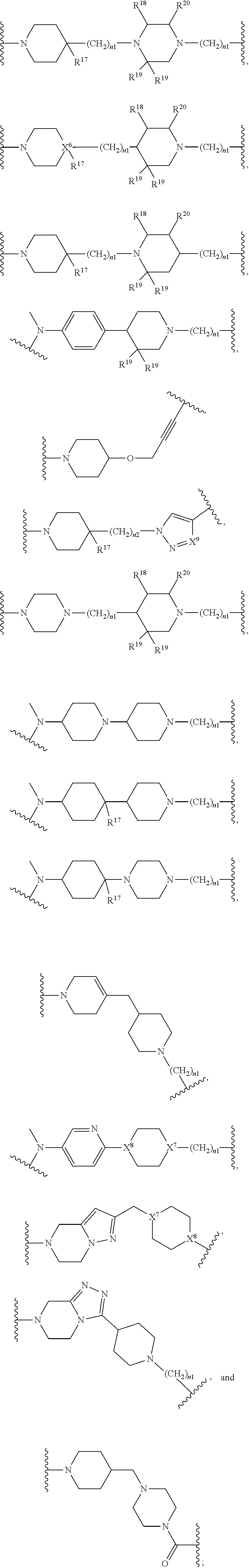

wherein n1 is selected from 0 and 1;
n2 is selected from 1 and 2;
R¹⁷ is selected from hydrogen and fluoro;
R¹⁸ is selected from hydrogen, fluoro, C₁₋₄alkyl wherein said C₁₋₄alkyl may optionally be substituted one or more times with fluoro or C₁₋₄alkoxy;
R¹⁹ is independently selected from hydrogen, C₁₋₄alkyl and fluoro;
R²⁰ is selected from hydrogen and C₁₋₄alkyl;
X⁶ is C;
X⁷ is CR²⁴ or N;
R²⁴ is selected from hydrogen and hydroxy; and
X⁸ and X⁹ are independently CH or N.

In some embodiments, L is selected from:

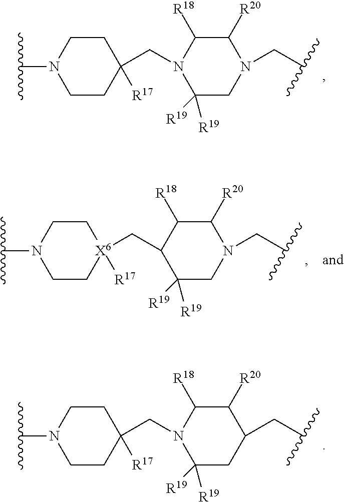

In some embodiments, L is selected from:

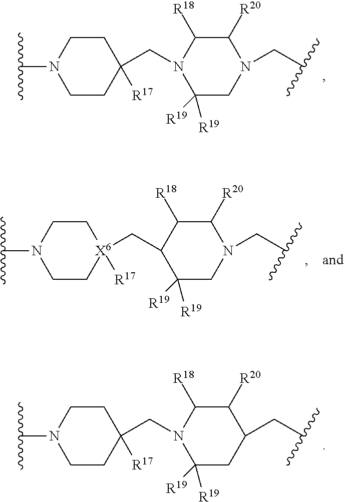

In some embodiments, at least one of the R¹⁷, R¹⁸, R¹⁹, and R²⁰ of L moiety is not hydrogen. In some embodiments, R″ is hydrogen and at least one of the R¹⁸, R¹⁹, and R²⁰ of L moiety is not hydrogen.

In some embodiments, L is selected from:

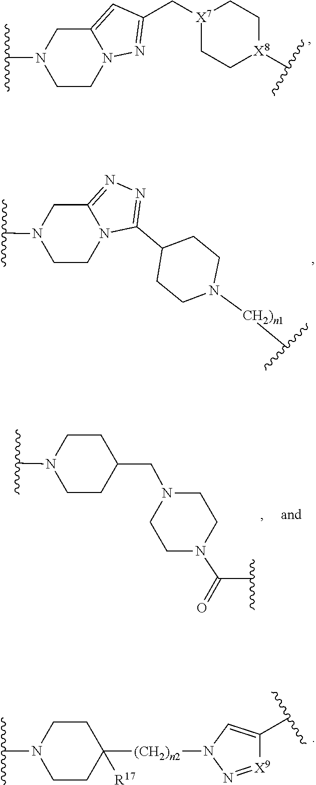

In some embodiments, A is CO and A-L is selected from:

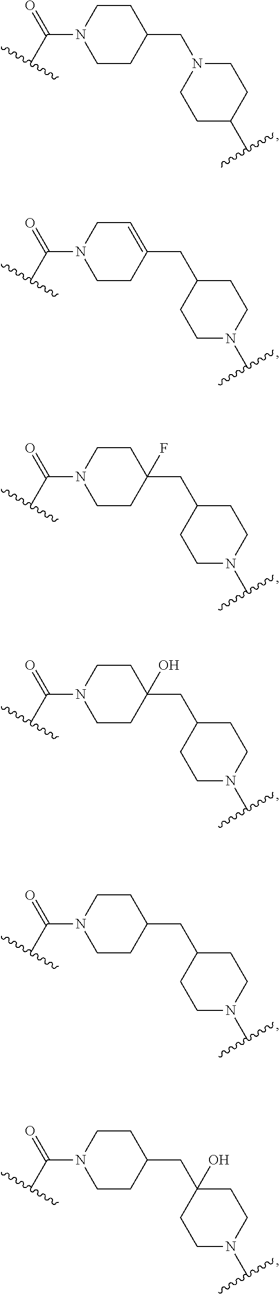 [SMILES: *C(=O)N1CCC(CN2CCC(*)CC2)CC1 ; *C(=O)N1CC=C(CC2CCN(*)CC2)CC1 ; *C(=O)N1CCC(F)(CC2CCN(*)CC2)CC1 ; *C(=O)N1CCC(O)(CC2CCN(*)CC2)CC1 ; *C(=O)N1CCC(CC2CCN(*)CC2)CC1 ; *C(=O)N1CCC(CC2(O)CCN(*)CC2)CC1]

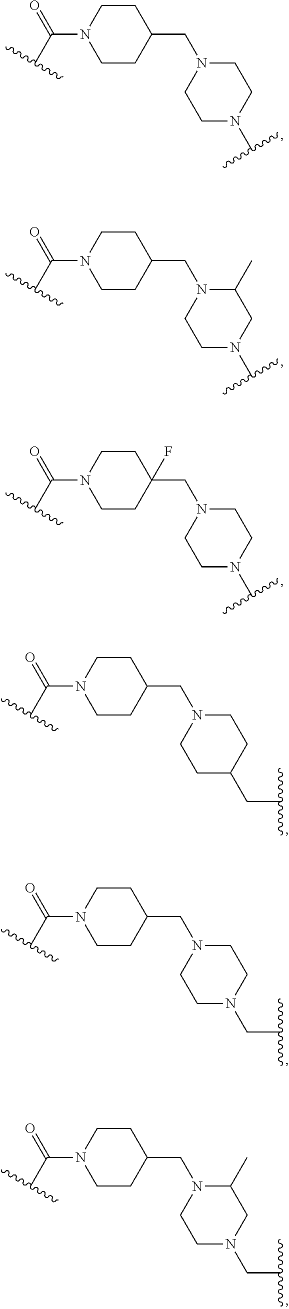 [SMILES: *C(=O)N1CCC(CN2CCN(*)CC2)CC1 ; *C(=O)N1CCC(CN2CCN(*)CC2C)CC1 ; *C(=O)N1CCC(F)(CN2CCN(*)CC2)CC1 ; *CC1CCN(CC2CCN(C(*)=O)CC2)CC1 ; *CN1CCN(CC2CCN(C(*)=O)CC2)CC1 ; *CN1CCN(CC2CCN(C(*)=O)CC2)C(C)C1]

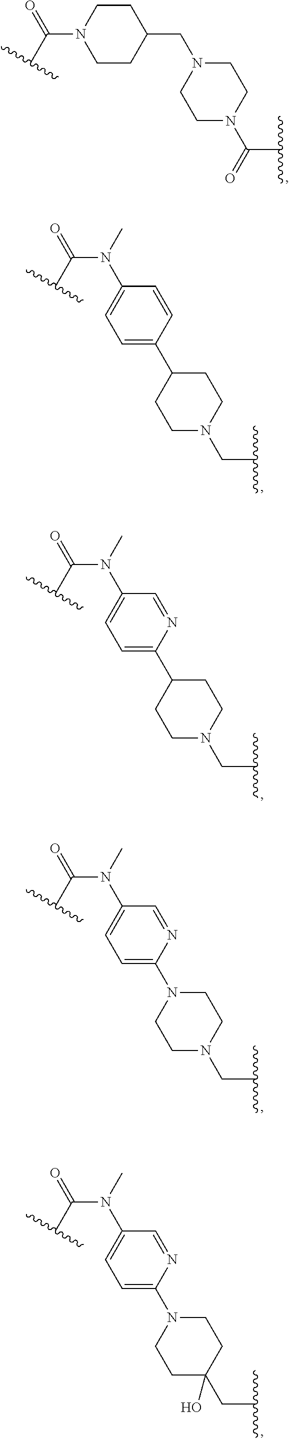 [SMILES: *C(=O)N1CCC(CN2CCN(C(*)=O)CC2)CC1 ; *CN1CCC(c2ccc(N(C)C(*)=O)cc2)CC1 ; *CN1CCC(c2ccc(N(C)C(*)=O)cn2)CC1 ; *CN1CCN(c2ccc(N(C)C(*)=O)cn2)CC1 ; *CC1(O)CCN(c2ccc(N(C)C(*)=O)cn2)CC1]

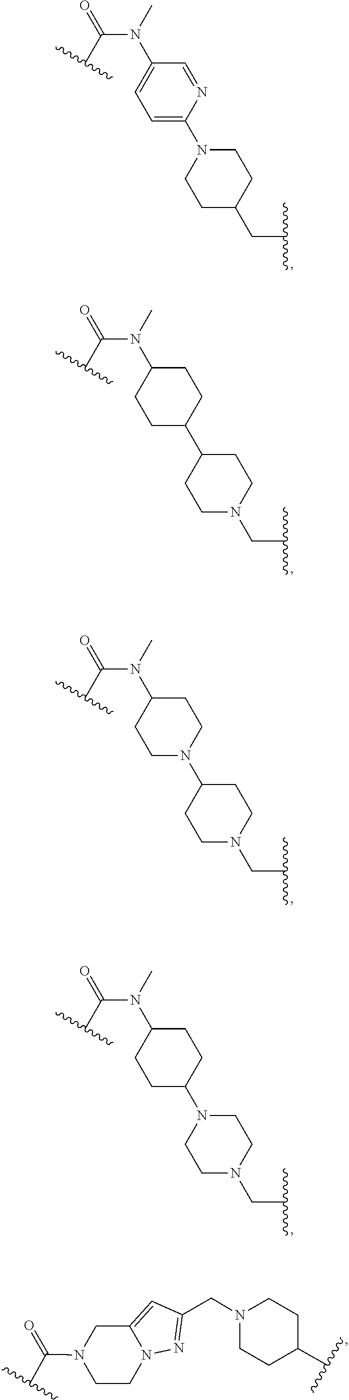 [SMILES: *CC1CCN(c2ccc(N(C)C(*)=O)cn2)CC1 ; *CN1CCC(C2CCC(N(C)C(*)=O)CC2)CC1 ; *CN1CCC(N2CCC(N(C)C(*)=O)CC2)CC1 ; *CN1CCN(C2CCC(N(C)C(*)=O)CC2)CC1 ; *C(=O)N1CCn2nc(CN3CCC(*)CC3)cc2C1]

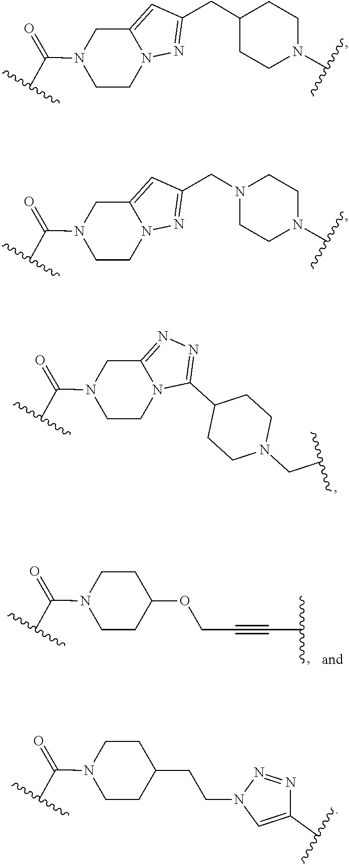 [SMILES: *C(=O)N1CCn2nc(CC3CCN(*)CC3)cc2C1 ; *C(=O)N1CCn2nc(CN3CCN(*)CC3)cc2C1 ; *CN1CCC(c2nnc3n2CCN(C(*)=O)C3)CC1 ; *C#CCOC1CCN(C(*)=O)CC1 ; *C(=O)N1CCC(CCn2cc(*)nn2)CC1]

In some embodiments, A is CO.

In some embodiments, A is

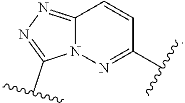 [SMILES: *c1ccc2nnc(*)n2n1]

In some embodiments, L is selected from:

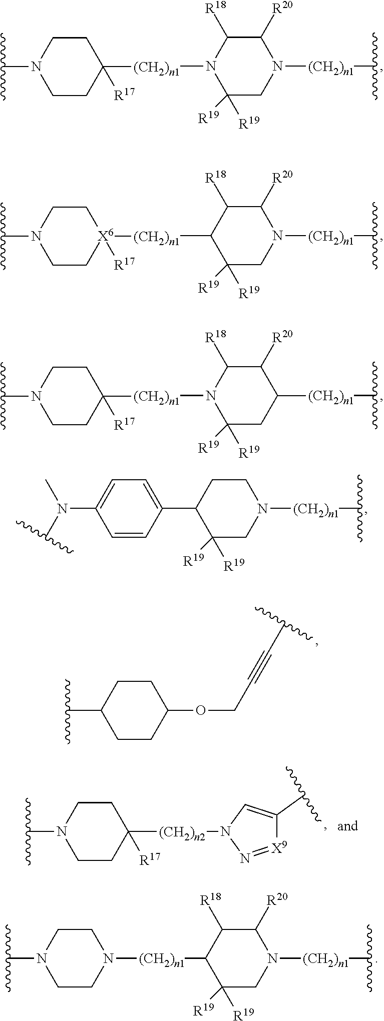

wherein each n1 is independently selected from 0 and 1;
n2 is selected from 1 and 2;
R¹⁷ is selected from hydrogen and fluoro;
R¹⁸ is selected from hydrogen, fluoro, and C₁₋₄alkyl, wherein said C₁₋₄alkyl may optionally be substituted one or more times with fluoro or C₁₋₄alkoxy;
each R¹⁹ is independently selected from hydrogen, C₁₋₄alkyl, and fluoro;
R²⁰ is selected from hydrogen and C₁₋₄alkyl;
X⁶ is C; and
X⁹ is CH or N.

In some embodiments, L is selected from:

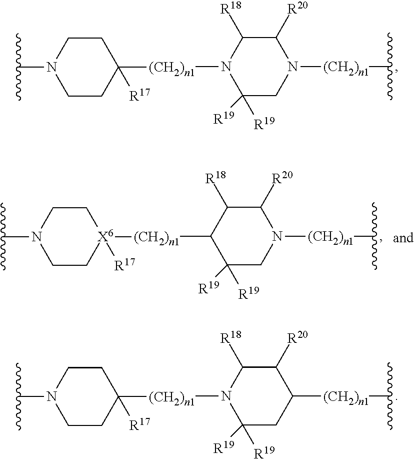

In some embodiments, L is

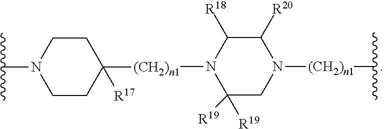

In some embodiments, L is

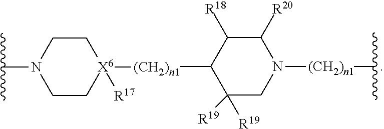

In some embodiments, L is

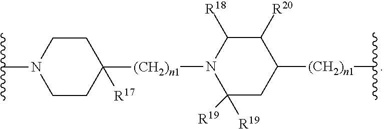

In some embodiments, L is

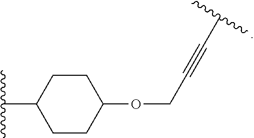 [SMILES: *C#CCOC1CCC(*)CC1]

In some embodiments, L is

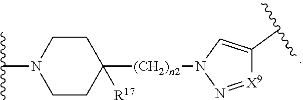

In some embodiments, L is

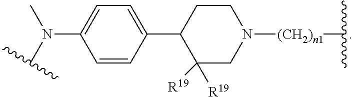

In some embodiments, L is

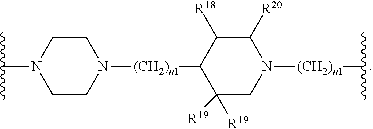

In some embodiments, at least one of the R¹⁷, R¹⁸, R¹⁹, and R²⁰ of L moiety is not hydrogen.

In some embodiments, R¹⁷ is hydrogen and at least one of the R¹, R¹⁹, and R²⁰ of L moiety is not hydrogen.

In some embodiments, A-L is selected from:

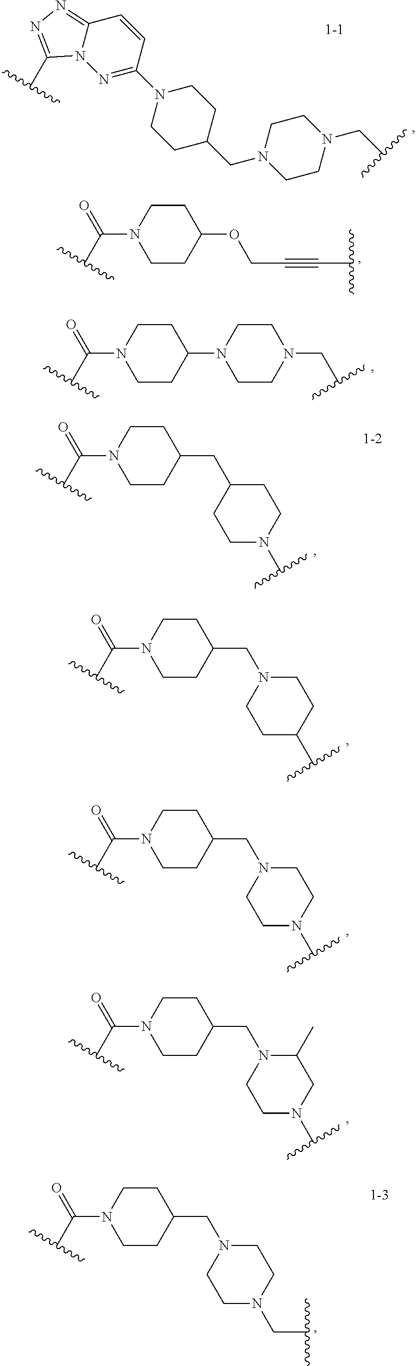

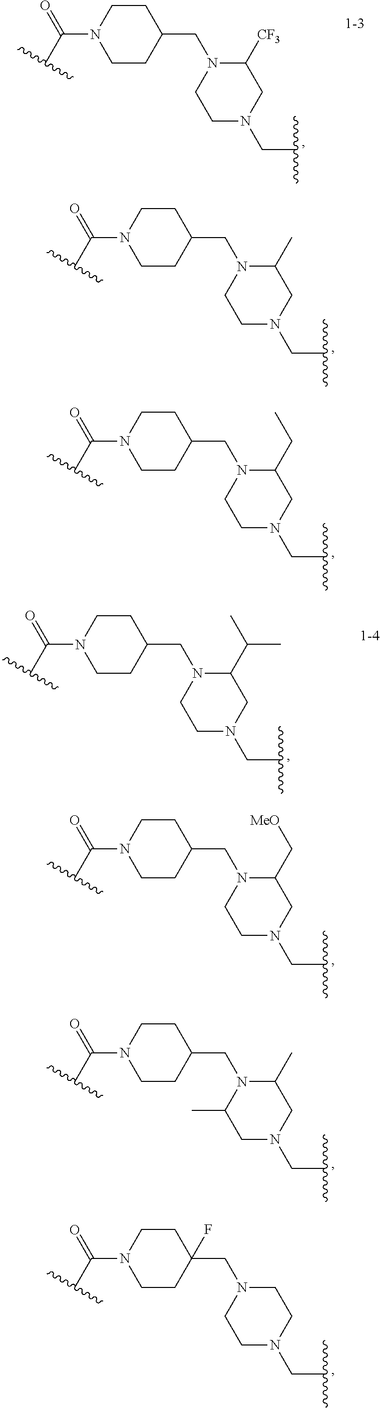

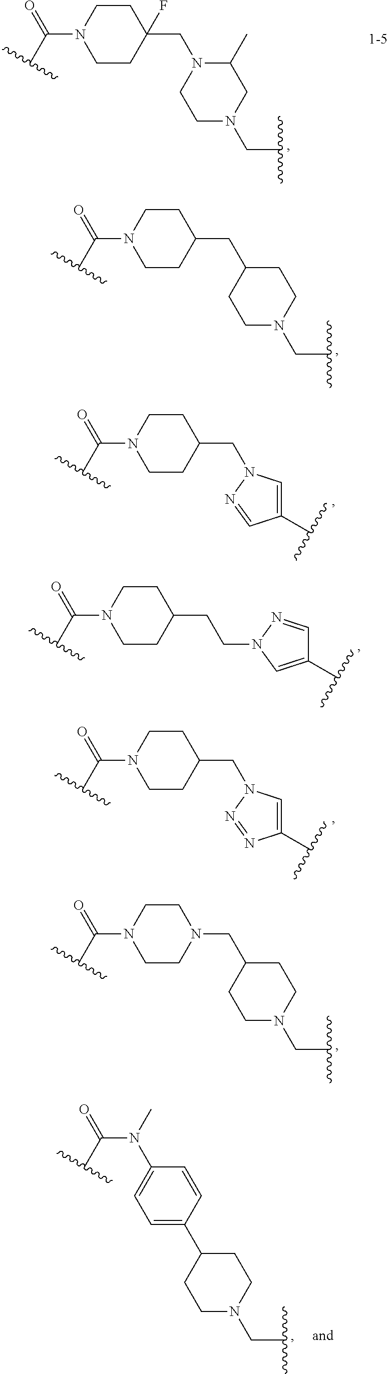

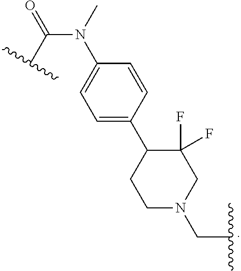 [SMILES: *CN1CCC(c2ccc(N(C)C(*)=O)cc2)C(F)(F)C1]

### Moiety LBM

In some embodiments, LBM is

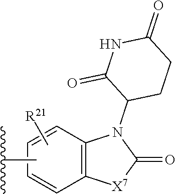

wherein X⁷ is O, NR²², or CR²³R²³;
R²¹ is selected from hydrogen, fluoro, and chloro;
R²² is selected from hydrogen, C₁₋₄alkyl, deuterated C₁₋₄alkyl, and C₃₋₄ cycloalkyl, wherein said C₁₋₄alkyl may optionally be substituted one or more times with fluoro, hydroxy, C₁₋₄alkoxy, or C₃₋₄ cycloalkyl; and
each R²³ is independently selected from hydrogen, halogen, and C₁₋₄alkyl; or
both R²³ together with the carbon they are attached to form a C₃₋₆ cycloalkyl or 3-6 membered heterocyclic ring, wherein the heterocyclic ring contains one to two heteroatoms selected from O.

In some embodiments, X⁷ is NR²²; R²² is selected from hydrogen, C₁₋₄alkyl, deuterated C₁₋₄alkyl, and C₃₋₄ cycloalkyl, wherein said C₁₋₄alkyl may optionally be substituted one or more times with fluoro, hydroxy, C₁₋₄alkoxy, or C₃₋₄ cycloalkyl.

In some embodiments, X⁷ is NR²²; R²² is selected from C₁₋₄alkyl, deuterated C₁₋₄alkyl, and C₃₋₄ cycloalkyl, wherein said C₁₋₄alkyl may optionally be substituted one or more times with fluoro, hydroxy, C₁₋₄alkoxy, or C₃₋₄ cycloalkyl.

In some embodiments, X⁷ is NR²²; R²² is selected from C₁₋₄alkyl and C₃₋₄ cycloalkyl.

In some embodiments, X⁷ is NR²² and R²² is methyl.

In some embodiments, X⁷ is NR²² and R²² is cyclopropyl.

In some embodiments, X⁷ is CR²³R²³; each R²³ is independently selected from halogen and C₁₋₄alkyl; or both R²³ together with the carbon they are attached to form a C₃₋₆ cycloalkyl or 3-6 membered heterocyclic ring, wherein the heterocyclic ring contains one to two heteroatoms selected from O.

In some embodiments, X⁷ is CR²³R²³; each R²³ is independently selected from hydrogen and C₁₋₄alkyl.

In some embodiments, X⁷ is CR²³R²³, and both R²³ are methyl or both R²³ are fluoro.

In some embodiments, X⁷ is CR²³R²³, and both R²³ are methyl.

In some embodiments, X⁷ is CR²³R²³; both R²³ together with the carbon they are attached to form a C₃₋₆ cycloalkyl or 3-6 membered heterocyclic ring, wherein the heterocyclic ring contains one to two heteroatoms selected from O.

In some embodiments, X⁷ is CR²³R²³, and both R²³ together with the carbon they are attached to form a C₃₋₆ cycloalkyl.

In some embodiments, X⁷ is CR²³R²³, and both R²³ together with the carbon they are attached to form a 3-6 membered heterocyclic ring, wherein the heterocyclic ring contains one heteroatom that is O.

In some embodiments, R²¹ is hydrogen or fluoro.

In some embodiments, LBM is selected from:

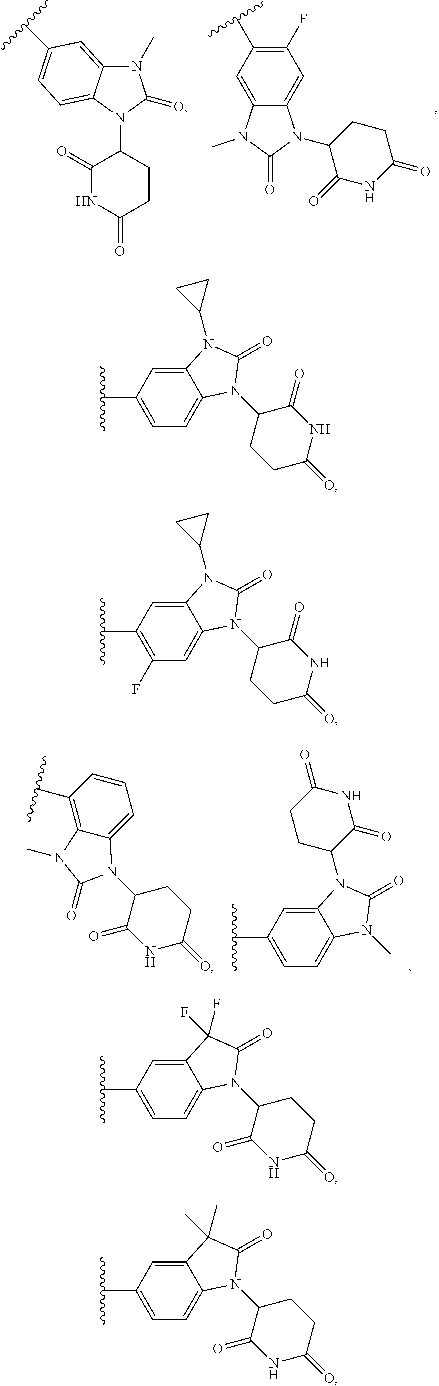

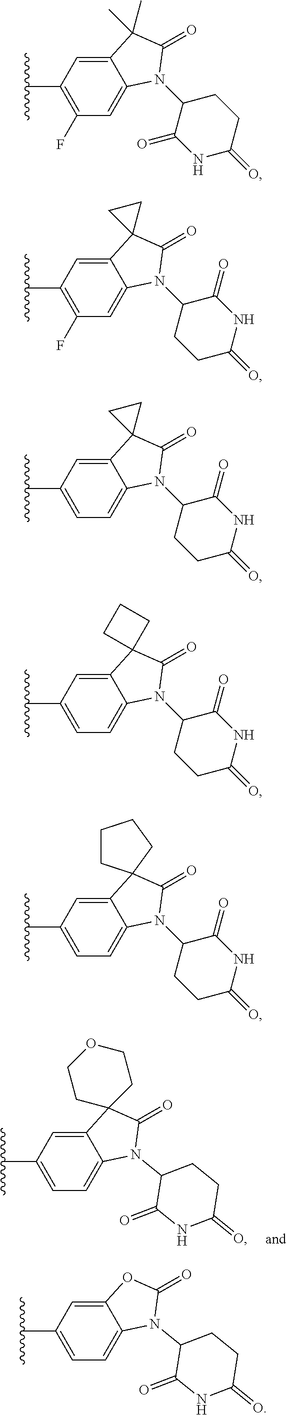

In some embodiments, LBM is

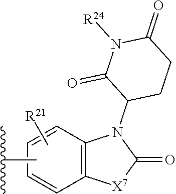

wherein X⁷ is O, NR²², or CR²³R²³;
R²¹ is selected from hydrogen, fluoro, and chloro;
R²² is selected fro, hydrogen, C₁₋₄alkyl, deuterated C₁₋₄alkyl, and C₃₋₄ cycloalkyl, wherein said C₁₋₄alkyl may optionally be substituted one or more times with fluoro, hydroxy, C₁₋₄alkoxy, or C₃₋₄ cycloalkyl;
each R²³ is independently selected from hydrogen and C₁₋₄alkyl, or both instances of R²³ together with the atom to which they are attached form a 3 or 4 membered carbocyclic ring which may be optionally substituted one or more times with a C₁₋₄alkyl, fluoro, or hydroxy; and
R²⁴ is selected from hydrogen and C₁₋₄alkyl.

In some embodiments, X⁷ is NR²²; R²² is selected from hydrogen, C₁₋₄alkyl, deuterated C₁₋₄alkyl, and C₃₋₄ cycloalkyl; and wherein said C₁₋₄alkyl may optionally be substituted one or more times with fluoro, hydroxy, C₁₋₄alkoxy, or C₃₋₄ cycloalkyl.

In some embodiments, X⁷ is NR²²; R²² is selected from C₁₋₄alkyl, deuterated C₁₋₄alkyl, and C₃₋₄ cycloalkyl; and wherein said C₁₋₄alkyl may optionally be substituted one or more times with fluoro, hydroxy, C₁₋₄alkoxy, or C₃₋₄ cycloalkyl.

In some embodiments, X⁷ is NR²²; R²² is selected from C₁₋₄alkyl and deuterated C₁₋₄alkyl; and wherein said C₁₋₄alkyl may optionally be substituted one or more times with fluoro, hydroxy, C₁₋₄alkoxy, or C₃₋₄ cycloalkyl.

In some embodiments, X⁷ is NR²²; and R²² is C₃₋₄ cycloalkyl.

In some embodiments, X⁷ is CR²³R²³; both R²³ are C₁₋₄alkyl; or two R²³ together with the atom to which they are attached form a 3 or 4 membered carbocyclic ring which may be optionally substituted one or more times with a C₁₋₄alkyl, fluoro, or hydroxy. In some embodiments, X⁷ is CR²³R²³; each R²³ is independently selected from hydrogen and C₁₋₄alkyl.

In some embodiments, X⁷ is CR²³R²³; two R²³ together with the atom to which they are attached form a 3 or 4 membered carbocyclic ring which may be optionally substituted one or more times with a C₁₋₄alkyl, fluoro, or hydroxy.

In some embodiments, R²⁴ is hydrogen.

In some embodiments, R²¹ is hydrogen, chloro, or fluoro.

In some embodiments, R²¹ is hydrogen or fluoro.

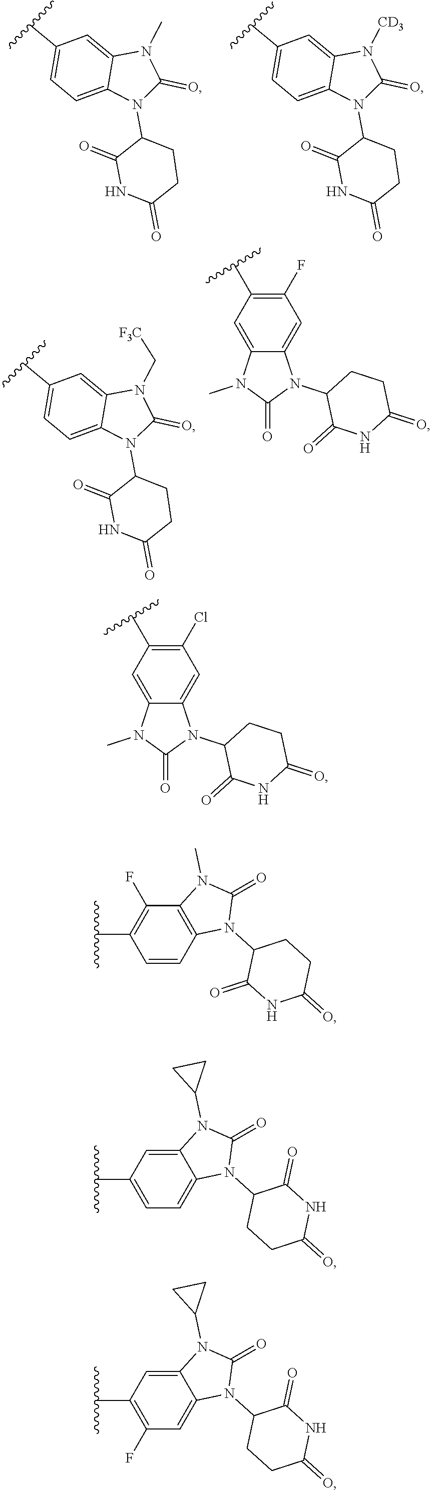

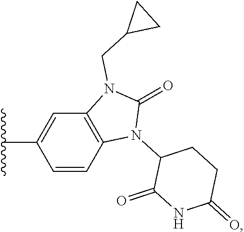 [SMILES: *c1ccc2c(c1)n(CC1CC1)c(=O)n2C1CCC(=O)NC1=O]

In some embodiments, LBM is selected from:

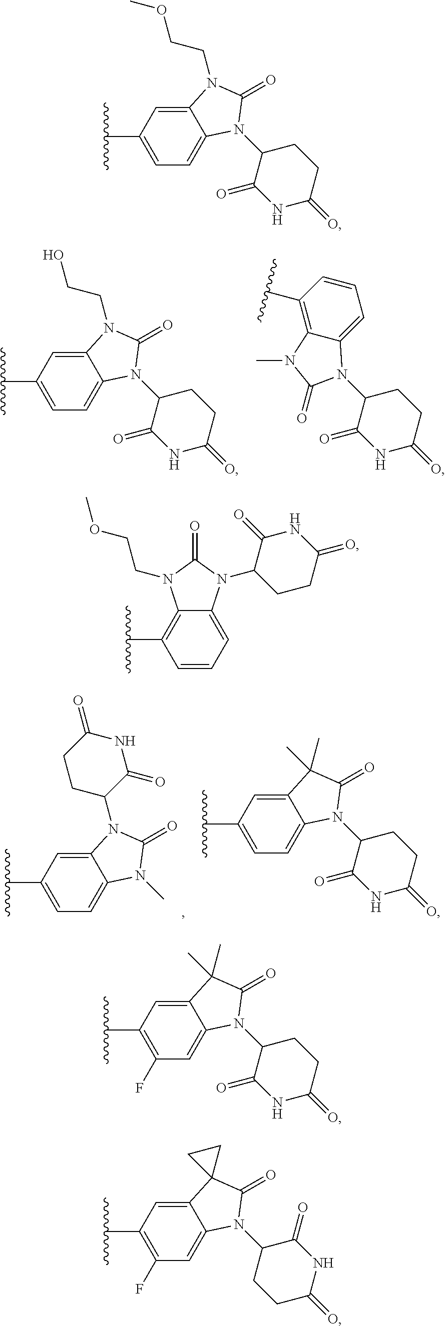

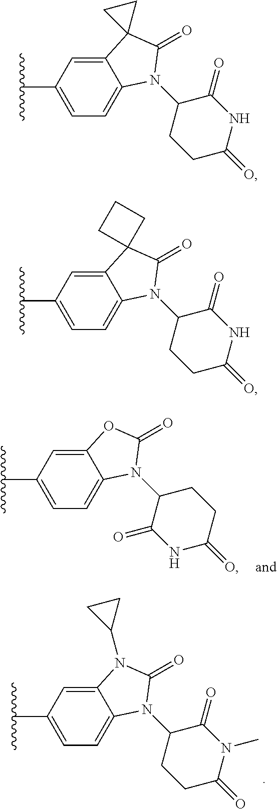 [SMILES: *c1ccc2c(c1)C1(CC1)C(=O)N2C1CCC(=O)NC1=O ; *c1ccc2c(c1)C1(CCC1)C(=O)N2C1CCC(=O)NC1=O ; *c1ccc2c(c1)oc(=O)n2C1CCC(=O)NC1=O ; "and" ; *c1ccc2c(c1)n(C1CC1)c(=O)n2C1CCC(=O)N(C)C1=O]

In some embodiments, LBM is selected from:

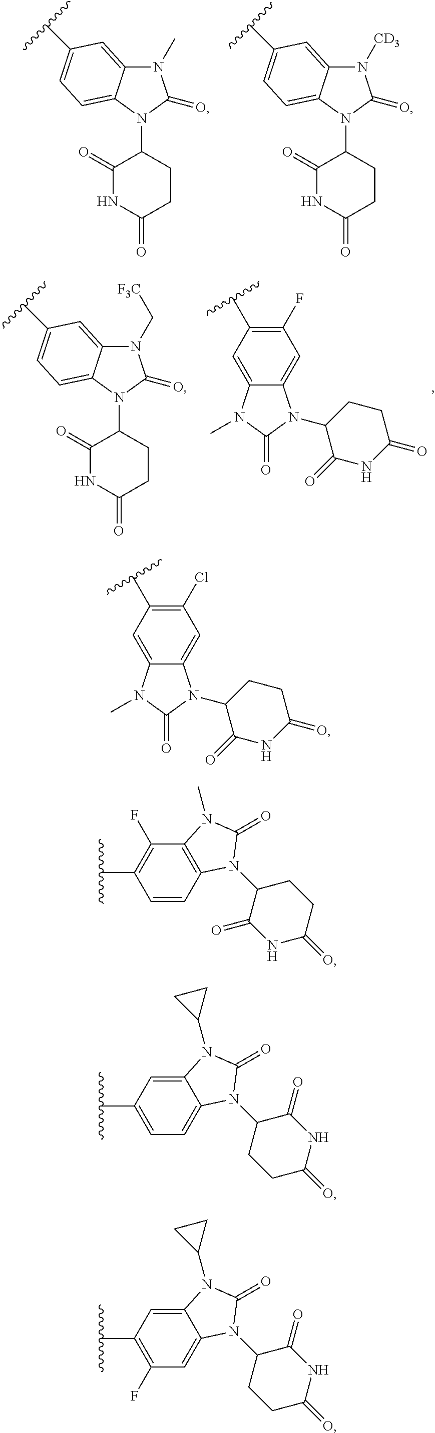

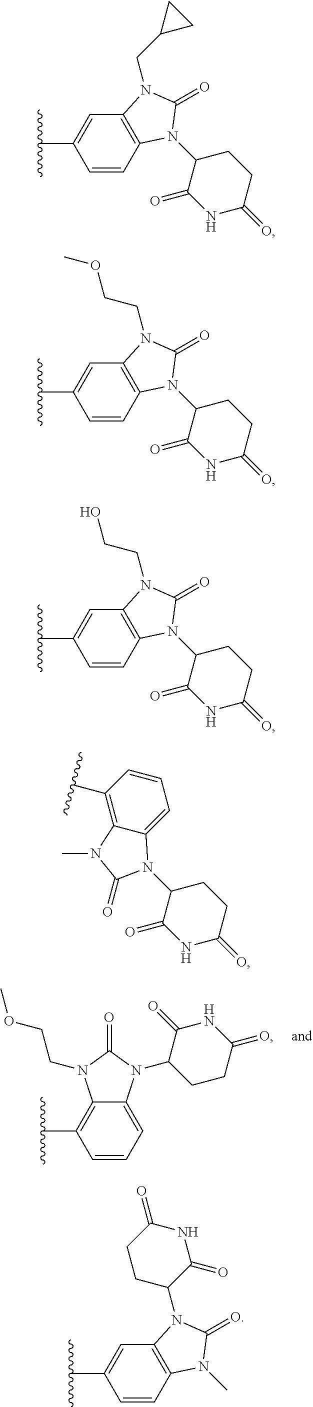

In some embodiments, LBM is selected from:

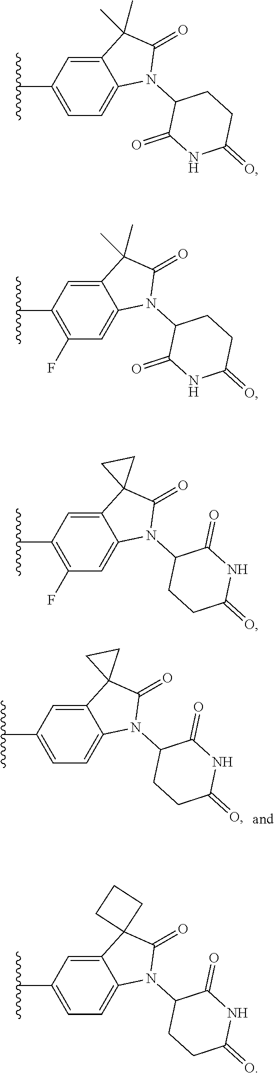 [SMILES: *c1ccc2c(c1)C(C)(C)C(=O)N2C1CCC(=O)NC1=O ; *c1cc2c(cc1F)N(C1CCC(=O)NC1=O)C(=O)C2(C)C ; *c1cc2c(cc1F)N(C1CCC(=O)NC1=O)C(=O)C21CC1 ; *c1ccc2c(c1)C1(CC1)C(=O)N2C1CCC(=O)NC1=O ; *c1ccc2c(c1)C1(CCC1)C(=O)N2C1CCC(=O)NC1=O]

In some embodiments, LBM is of the formula:

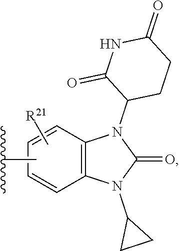

wherein R²¹ is hydrogen or fluoro.

### Moiety A-L-LAM

In some embodiments, A-L-LBM is

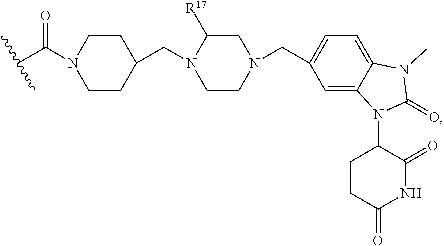

wherein R¹⁷ is as defined anywhere herein.

In some embodiments, A-L-LBM is

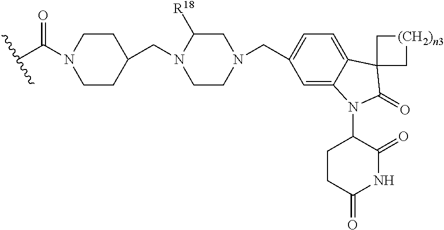

wherein R¹⁷ is as defined anywhere herein, and n3 is 0, 1, 2 or 3.

In some embodiments, A-L-LBM is of the formula:

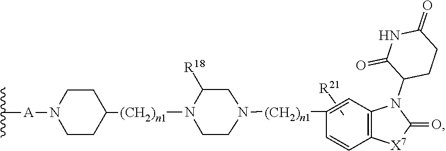

and wherein A is CO, n1 is 1, and R¹⁸ and R²¹ are as defined anywhere herein.

In some embodiments, A-L-LBM is of the formula:

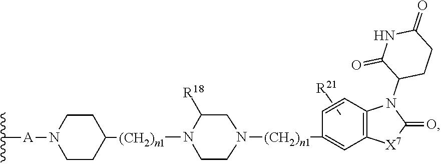

and wherein A is CO, n1 is 1, and X⁷, R¹⁸, and R²¹ are as defined anywhere herein.

In some embodiments, A-L-LBM is of the formula:

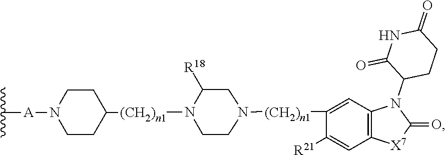

and wherein A is CO, n is 1, R¹⁸ is hydrogen or C₁₋₄alkyl, and R²¹ is hydrogen and fluoro.

In some embodiments, A-L-LBM is of the formula:

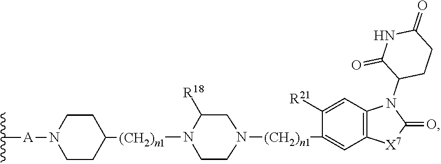

and wherein A is CO, n is 1, R¹⁸ is hydrogen or C₁₋₄alkyl, R²¹ is hydrogen, chloro, or fluoro, and X⁷ is as defined anywhere herein.

In some embodiments, provided is a compound selected from Table 1, or a pharmaceutically acceptable salt or stereoisomer thereof.

TABLE 1

| No. | Structure |
|---|---|
| 3e39 | 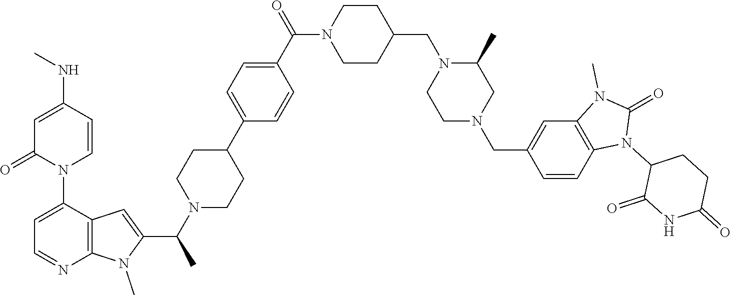 [SMILES: CNc1ccn(-c2ccnc3c2cc([C@H](C)N2CCC(c4ccc(C(=O)N5CCC(CN6CCN(Cc7ccc8c(c7)n(C)c(=O)n8C7CCC(=O)NC7=O)C[C@@H]6C)CC5)cc4)CC2)n3C)c(=O)c1] |
| 3e44 | 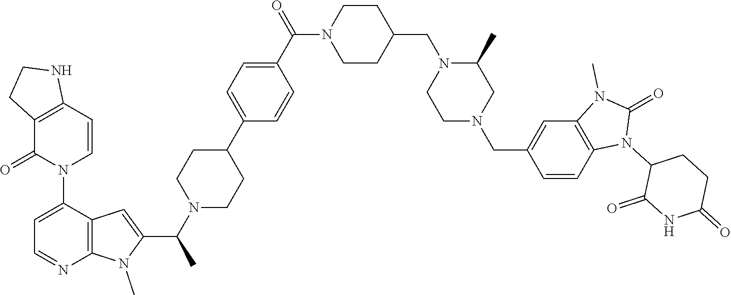 [SMILES: C[C@H]1CN(Cc2ccc3c(c2)n(C)c(=O)n3C2CCC(=O)NC2=O)CCN1CC1CCN(C(=O)c2ccc(C3CCN([C@@H](C)c4cc5c(-n6ccc7c(c6=O)CCN7)ccnc5n4C)CC3)cc2)CC1] |
| 3h10 | 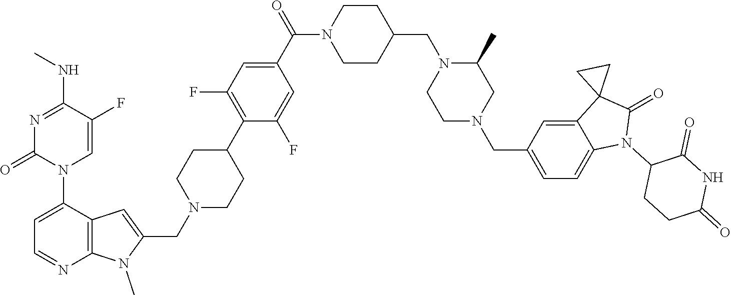 [SMILES: CNc1nc(=O)n(-c2ccnc3c2cc(CN2CCC(c4c(F)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)cc1F] |
| 3h12 | 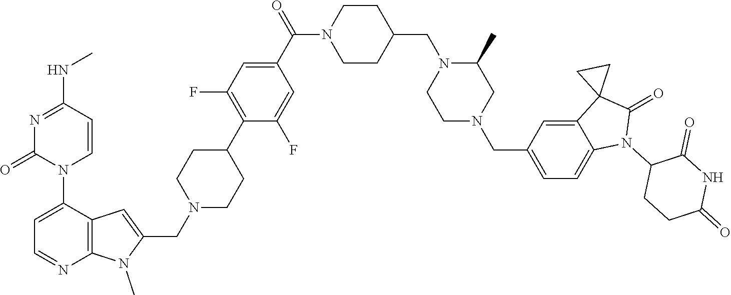 [SMILES: CNc1ccn(-c2ccnc3c2cc(CN2CCC(c4c(F)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)c(=O)n1] |
| 3u1 | 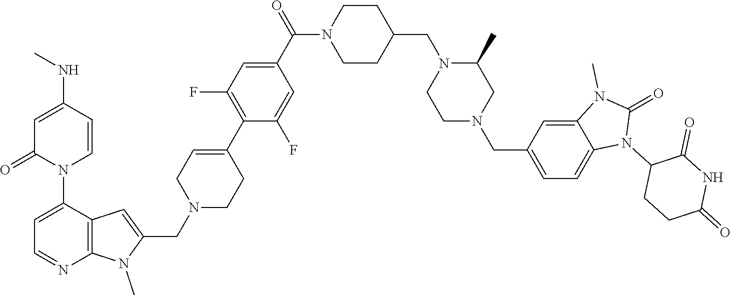 [SMILES: CNc1ccn(-c2ccnc3c2cc(CN2CC=C(c4c(F)cc(C(=O)N5CCC(CN6CCN(Cc7ccc8c(c7)n(C)c(=O)n8C7CCC(=O)NC7=O)C[C@@H]6C)CC5)cc4F)CC2)n3C)c(=O)c1] |
| 3u10 | 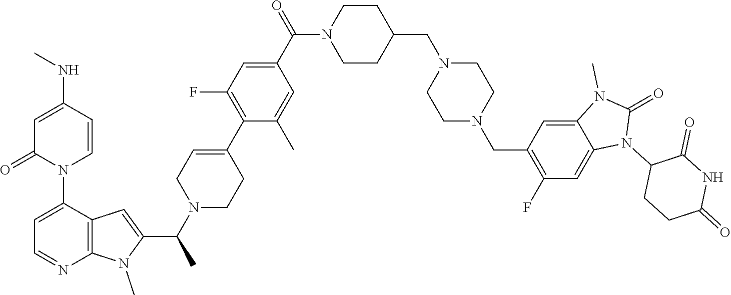 [SMILES: CNc1ccn(-c2ccnc3c2cc([C@H](C)N2CC=C(c4c(C)cc(C(=O)N5CCC(CN6CCN(Cc7cc8c(cc7F)n(C7CCC(=O)NC7=O)c(=O)n8C)CC6)CC5)cc4F)CC2)n3C)c(=O)c1] |
| 3u100 and 3u99 | 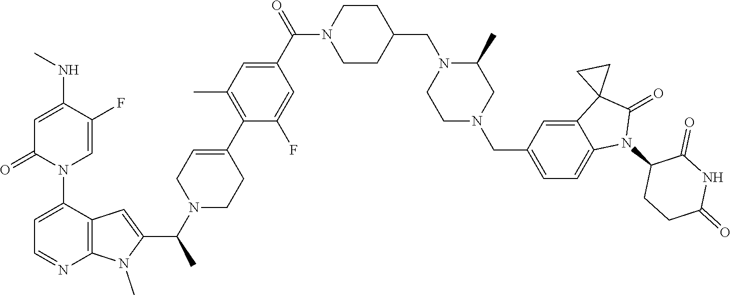 [SMILES: CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8[C@@H]7CCC(=O)NC7=O)C[C@@H]6C)CC5)cc4F)CC2)n3C)cc1F] |
| | 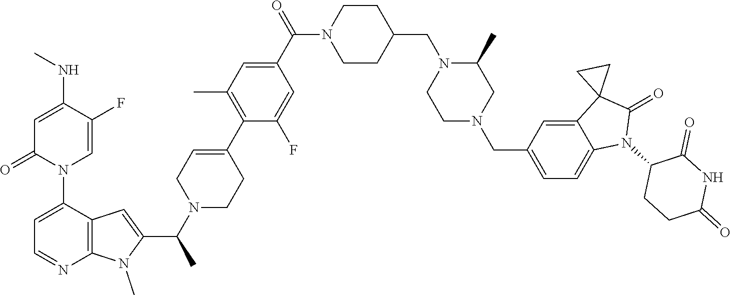 [SMILES: CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8[C@H]7CCC(=O)NC7=O)C[C@@H]6C)CC5)cc4F)CC2)n3C)cc1F] |
| 3u101 | 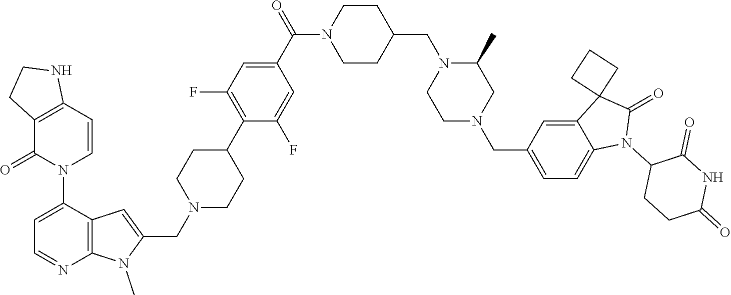 [SMILES: C[C@H]1CN(Cc2ccc3c(c2)C2(CCC2)C(=O)N3C2CCC(=O)NC2=O)CCN1CC1CCN(C(=O)c2cc(F)c(C3CCN(Cc4cc5c(-n6ccc7c(c6=O)CCN7)ccnc5n4C)CC3)c(F)c2)CC1] |
| 3u102 | 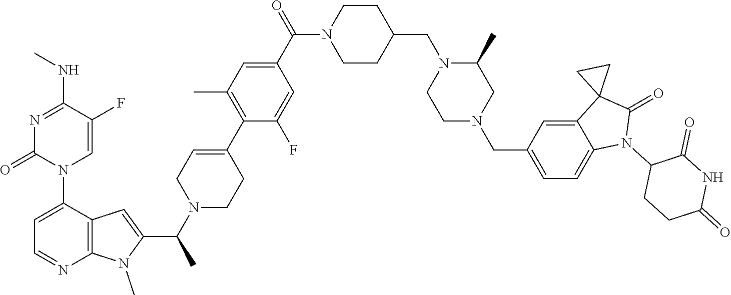 [SMILES: CNc1nc(=O)n(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)cc1F] |
| 3u103 | 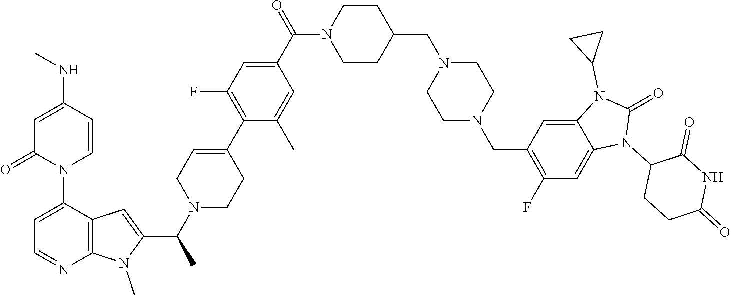 [SMILES: CNc1ccn(-c2ccnc3c2cc([C@H](C)N2CC=C(c4c(C)cc(C(=O)N5CCC(CN6CCN(Cc7cc8c(cc7F)n(C7CCC(=O)NC7=O)c(=O)n8C7CC7)CC6)CC5)cc4F)CC2)n3C)c(=O)c1] |
| 3u104 and 3u105 | 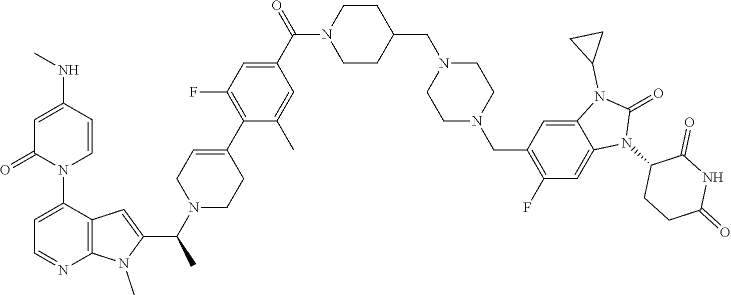 [SMILES: CNc1ccn(-c2ccnc3c2cc([C@H](C)N2CC=C(c4c(C)cc(C(=O)N5CCC(CN6CCN(Cc7cc8c(cc7F)n([C@H]7CCC(=O)NC7=O)c(=O)n8C7CC7)CC6)CC5)cc4F)CC2)n3C)c(=O)c1] |
| | 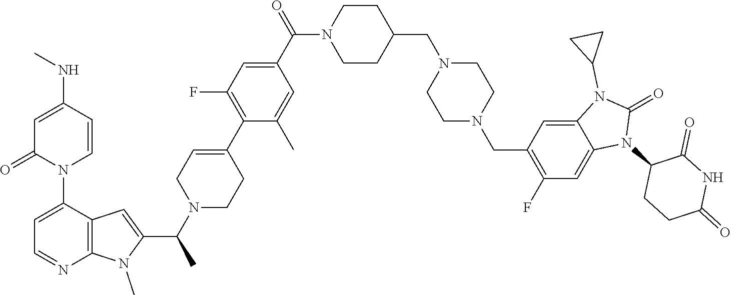 [SMILES: CNc1ccn(-c2ccnc3c2cc([C@H](C)N2CC=C(c4c(C)cc(C(=O)N5CCC(CN6CCN(Cc7cc8c(cc7F)n([C@@H]7CCC(=O)NC7=O)c(=O)n8C7CC7)CC6)CC5)cc4F)CC2)n3C)c(=O)c1] |
| 3u106 | 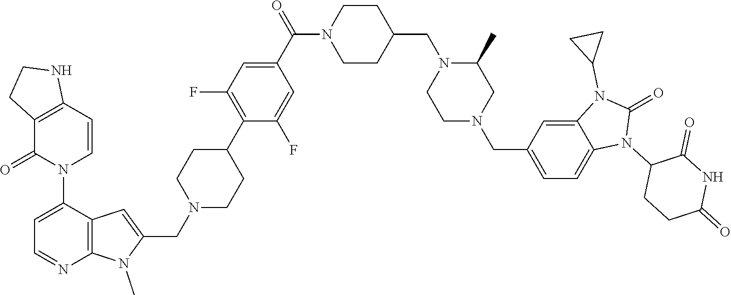 [SMILES: C[C@H]1CN(Cc2ccc3c(c2)n(C2CC2)c(=O)n3C2CCC(=O)NC2=O)CCN1CC1CCN(C(=O)c2cc(F)c(C3CCN(Cc4cc5c(-n6ccc7c(c6=O)CCN7)ccnc5n4C)CC3)c(F)c2)CC1] |
| 3u107 and 3u108 | 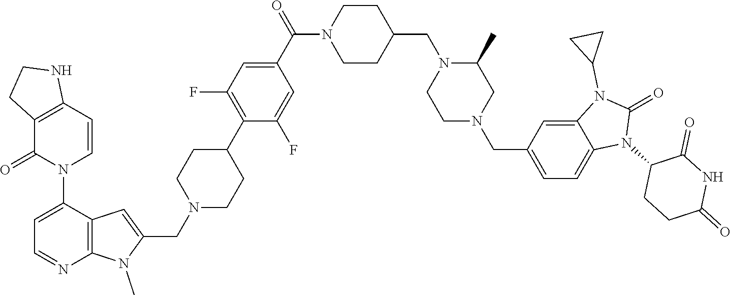 [SMILES: C[C@H]1CN(Cc2ccc3c(c2)n(C2CC2)c(=O)n3[C@H]2CCC(=O)NC2=O)CCN1CC1CCN(C(=O)c2cc(F)c(C3CCN(Cc4cc5c(-n6ccc7c(c6=O)CCN7)ccnc5n4C)CC3)c(F)c2)CC1] |
| | 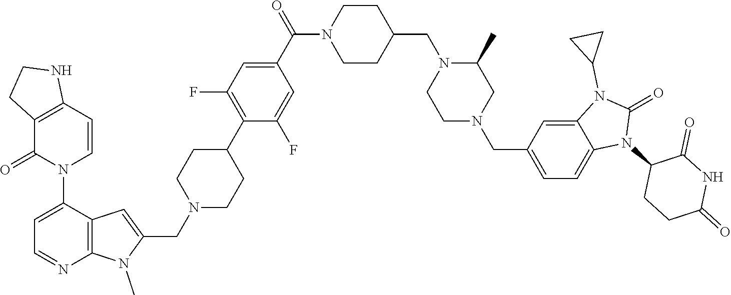 [SMILES: C[C@H]1CN(Cc2ccc3c(c2)n(C2CC2)c(=O)n3[C@@H]2CCC(=O)NC2=O)CCN1CC1CCN(C(=O)c2cc(F)c(C3CCN(Cc4cc5c(-n6ccc7c(c6=O)CCN7)ccnc5n4C)CC3)c(F)c2)CC1] |
| 3u109 | 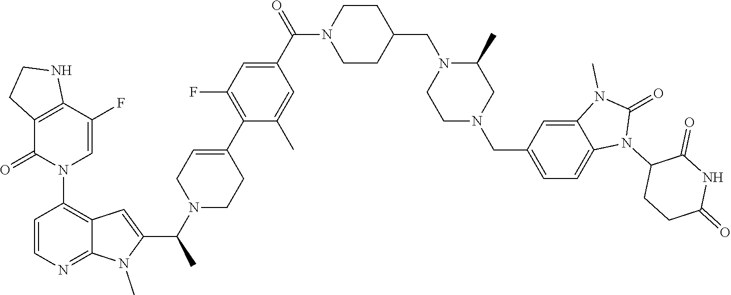 [SMILES: Cc1cc(C(=O)N2CCC(CN3CCN(Cc4ccc5c(c4)n(C)c(=O)n5C4CCC(=O)NC4=O)C[C@@H]3C)CC2)cc(F)c1C1=CCN([C@@H](C)c2cc3c(-n4cc(F)c5c(c4=O)CCN5)ccnc3n2C)CC1] |
| 3u11 and 3u12 | 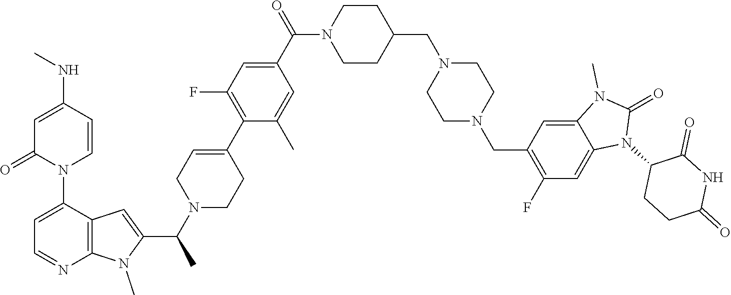 [SMILES: CNc1ccn(-c2ccnc3c2cc([C@H](C)N2CC=C(c4c(C)cc(C(=O)N5CCC(CN6CCN(Cc7cc8c(cc7F)n([C@H]7CCC(=O)NC7=O)c(=O)n8C)CC6)CC5)cc4F)CC2)n3C)c(=O)c1] |
| | 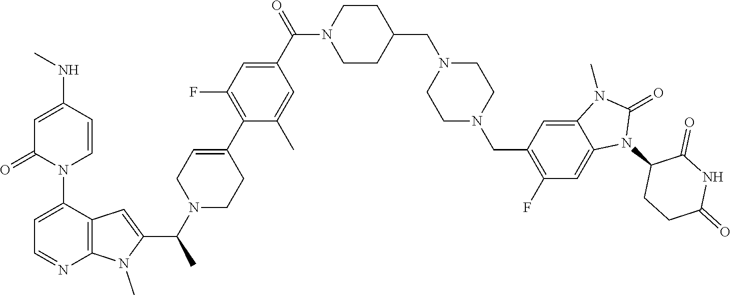 [SMILES: CNc1ccn(-c2ccnc3c2cc([C@H](C)N2CC=C(c4c(C)cc(C(=O)N5CCC(CN6CCN(Cc7cc8c(cc7F)n([C@@H]7CCC(=O)NC7=O)c(=O)n8C)CC6)CC5)cc4F)CC2)n3C)c(=O)c1] |
| 3u110 | 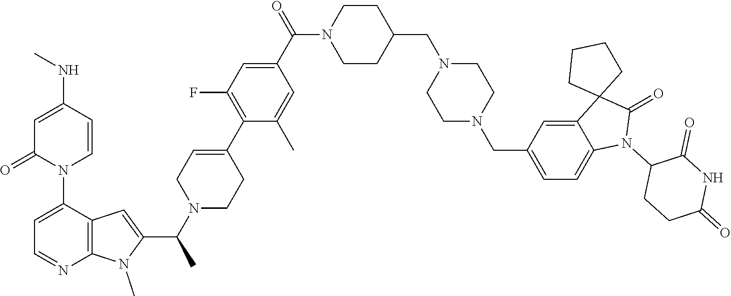 [SMILES: CNc1ccn(-c2ccnc3c2cc([C@H](C)N2CC=C(c4c(C)cc(C(=O)N5CCC(CN6CCN(Cc7ccc8c(c7)C7(CCCC7)C(=O)N8C7CCC(=O)NC7=O)CC6)CC5)cc4F)CC2)n3C)c(=O)c1] |
| 3u111 | 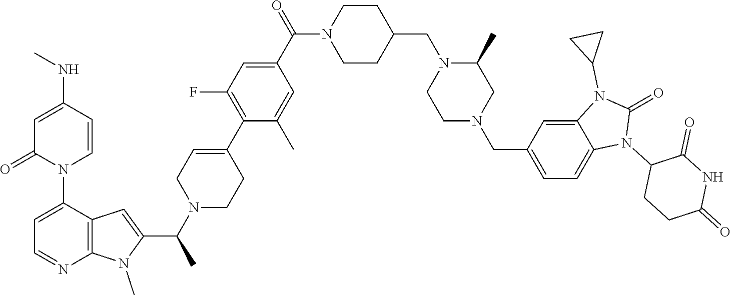 [SMILES: CNc1ccn(-c2ccnc3c2cc([C@H](C)N2CC=C(c4c(C)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)c(=O)c1] |
| 3u112 and 3u113 | 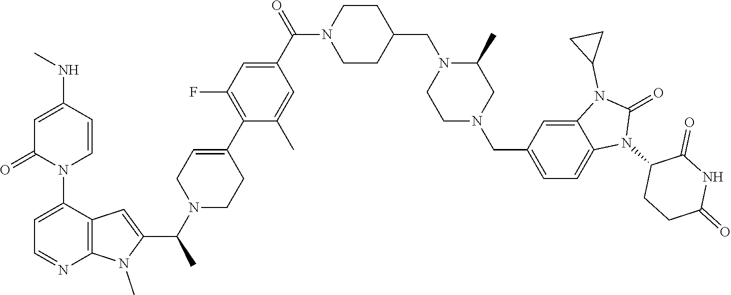 [SMILES: CNc1ccn(-c2ccnc3c2cc([C@H](C)N2CC=C(c4c(C)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)c(=O)c1] |
| | 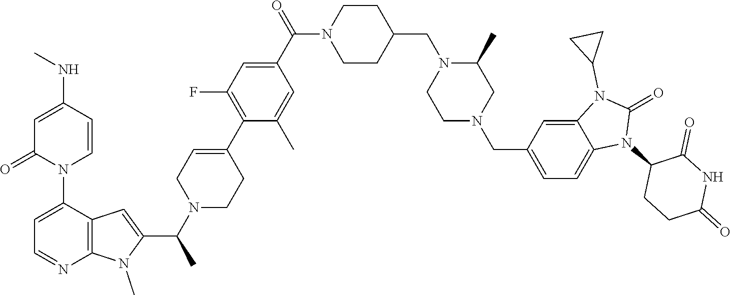 [SMILES: CNc1ccn(-c2ccnc3c2cc([C@H](C)N2CC=C(c4c(C)cc(C(=O)N5CCC(CN6CCN(Cc7ccc8c(c7)n(C7CC7)c(=O)n8[C@@H]7CCC(=O)NC7=O)C[C@@H]6C)CC5)cc4F)CC2)n3C)c(=O)c1] |
| 3u114 | 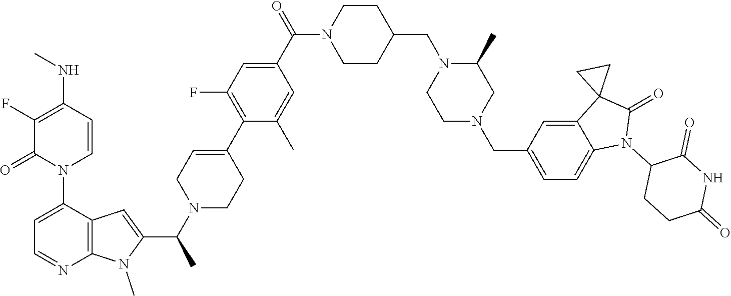 [SMILES: CNc1ccn(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)c(=O)c1F] |
| 3u115 | 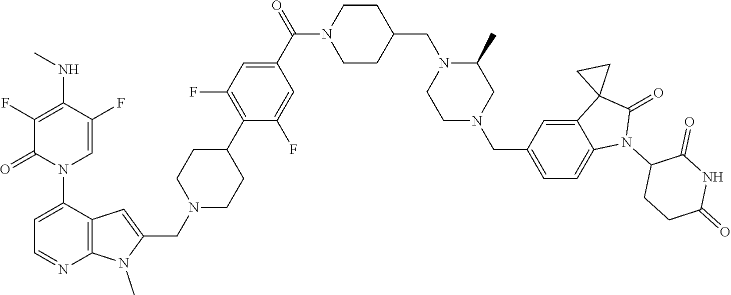 [SMILES: CNc1c(F)cn(-c2ccnc3c2cc(CN2CCC(c4c(F)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)c(=O)c1F] |
| 3u116 | 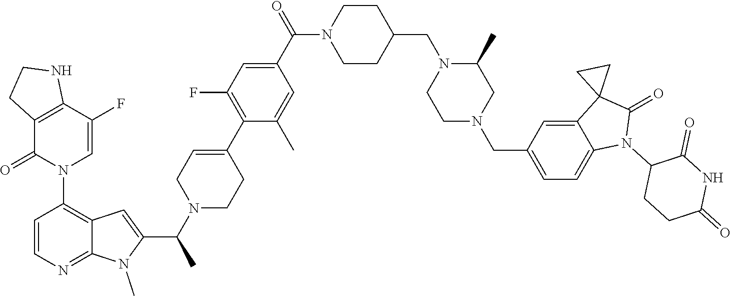 [SMILES: Cc1cc(C(=O)N2CCC(CN3CCN(Cc4ccc5c(c4)C4(CC4)C(=O)N5C4CCC(=O)NC4=O)C[C@@H]3C)CC2)cc(F)c1C1=CCN([C@@H](C)c2cc3c(-n4cc(F)c5c(c4=O)CCN5)ccnc3n2C)CC1] |
| 3u117 | 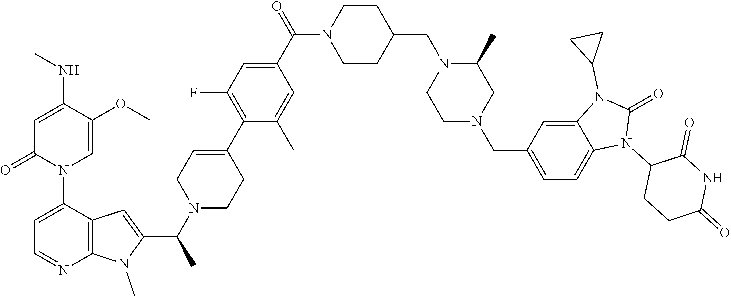 [SMILES: CNc1cc(=O)n(-c2ccnc3c2cc([C@H](C)N2CC=C(c4c(C)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)cc1OC] |
| 3u118 | 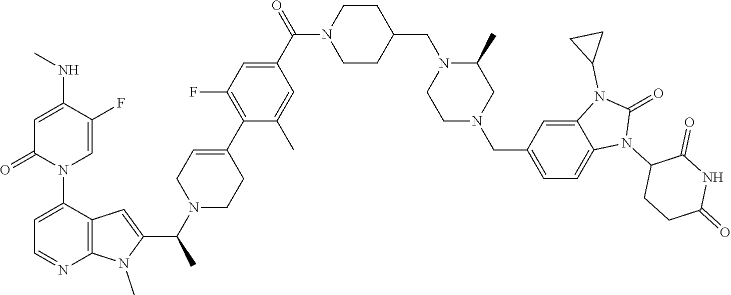 [SMILES: CNc1cc(=O)n(-c2ccnc3c2cc([C@H](C)N2CC=C(c4c(C)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)cc1F] |
| 3u119 and 3u120 | 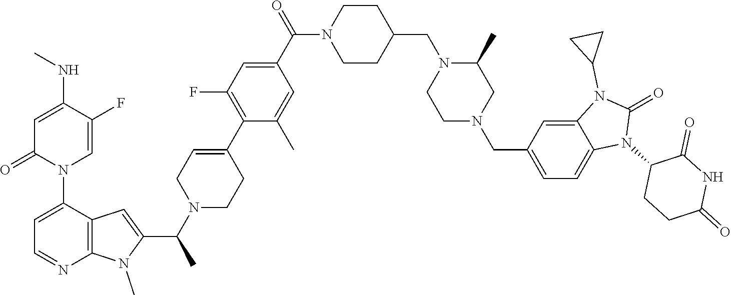 [SMILES: CNc1cc(=O)n(-c2ccnc3c2cc([C@H](C)N2CC=C(c4c(C)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)cc1F] |
| | 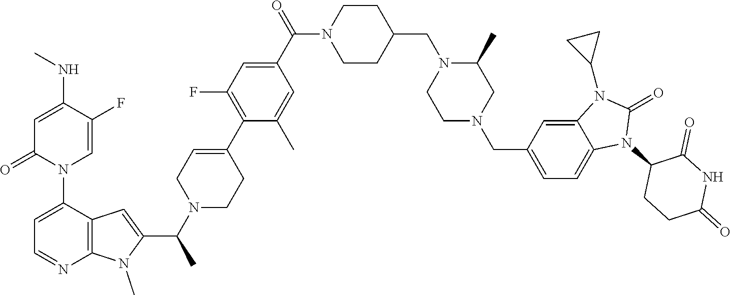 [SMILES: CNc1cc(=O)n(-c2ccnc3c2cc([C@H](C)N2CC=C(c4c(C)cc(C(=O)N5CCC(CN6CCN(Cc7ccc8c(c7)n(C7CC7)c(=O)n8[C@@H]7CCC(=O)NC7=O)C[C@@H]6C)CC5)cc4F)CC2)n3C)cc1F] |
| 3u121 | 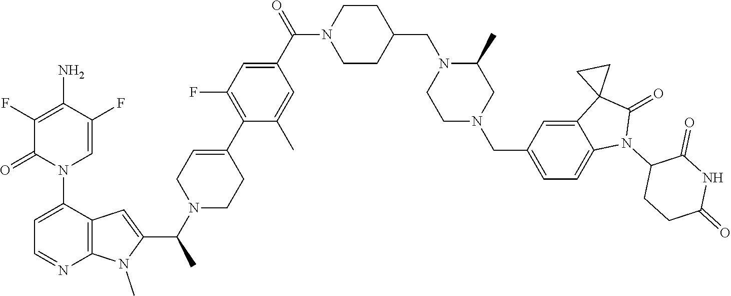 [SMILES: Cc1cc(C(=O)N2CCC(CN3CCN(Cc4ccc5c(c4)C4(CC4)C(=O)N5C4CCC(=O)NC4=O)C[C@@H]3C)CC2)cc(F)c1C1=CCN([C@@H](C)c2cc3c(-n4cc(F)c(N)c(F)c4=O)ccnc3n2C)CC1] |
| 3u122 | 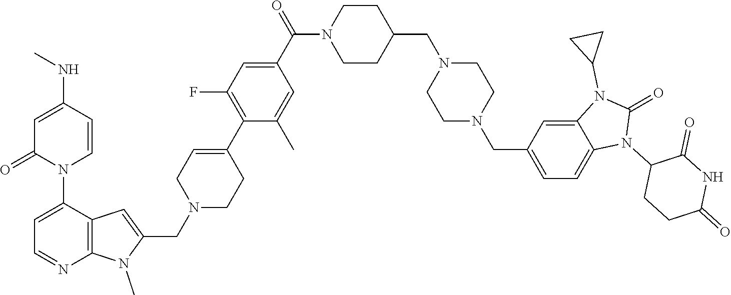 [SMILES: CNc1ccn(-c2ccnc3c2cc(CN2CC=C(c4c(C)cc(C(=O)N5CCC(CN6CCN(Cc7ccc8c(c7)n(C7CC7)c(=O)n8C7CCC(=O)NC7=O)CC6)CC5)cc4F)CC2)n3C)c(=O)c1] |
| 3u123 and 3u124 | 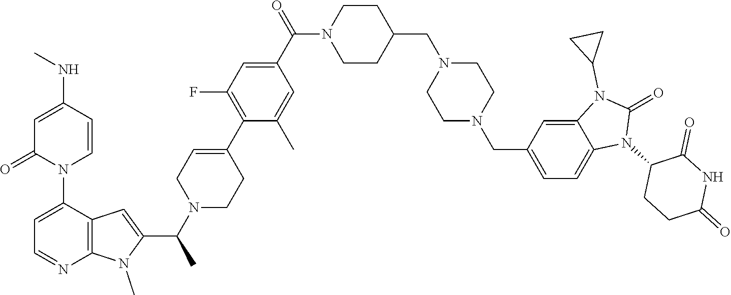 [SMILES: CNc1ccn(-c2ccnc3c2cc([C@H](C)N2CC=C(c4c(C)cc(C(=O)N5CCC(CN6CCN(Cc7ccc8c(c7)n(C7CC7)c(=O)n8[C@H]7CCC(=O)NC7=O)CC6)CC5)cc4F)CC2)n3C)c(=O)c1] |
| | 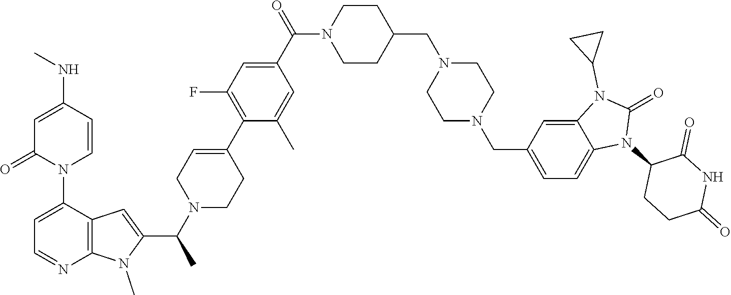 [SMILES: CNc1ccn(-c2ccnc3c2cc([C@H](C)N2CC=C(c4c(C)cc(C(=O)N5CCC(CN6CCN(Cc7ccc8c(c7)n(C7CC7)c(=O)n8[C@@H]7CCC(=O)NC7=O)CC6)CC5)cc4F)CC2)n3C)c(=O)c1] |
| 3u125 | 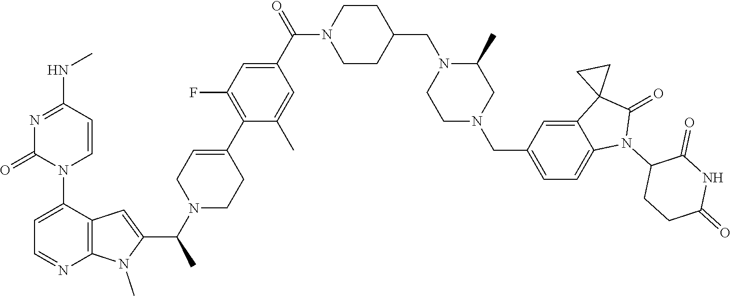 [SMILES: CNc1ccn(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)c(=O)n1] |
| 3u126 | 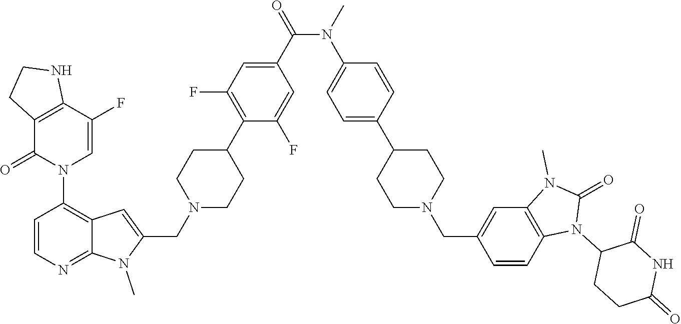 [SMILES: CN(C(=O)c1cc(F)c(C2CCN(Cc3cc4c(-n5cc(F)c6c(c5=O)CCN6)ccnc4n3C)CC2)c(F)c1)c1ccc(C2CCN(Cc3ccc4c(c3)n(C)c(=O)n4C3CCC(=O)NC3=O)CC2)cc1] |
| 3u127 | 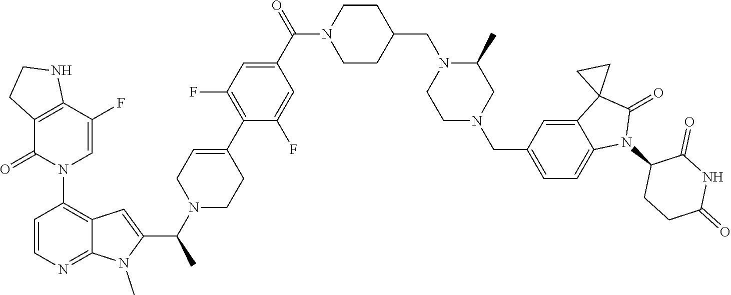 [SMILES: C[C@H]1CN(Cc2ccc3c(c2)C2(CC2)C(=O)N3[C@@H]2CCC(=O)NC2=O)CCN1CC1CCN(C(=O)c2cc(F)c(C3=CCN([C@@H](C)c4cc5c(-n6cc(F)c7c(c6=O)CCN7)ccnc5n4C)CC3)c(F)c2)CC1] |
| 3u128 | 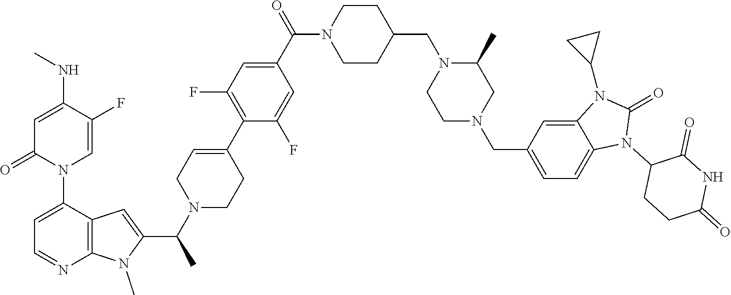 [SMILES: CNc1cc(=O)n(-c2ccnc3c2cc([C@H](C)N2CC=C(c4c(F)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)cc1F] |
| 3u129 and 3u130 | 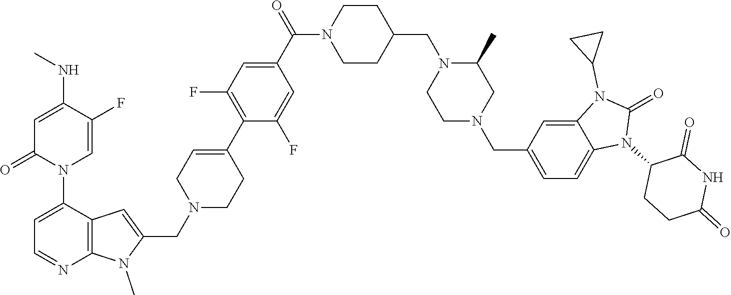 [SMILES: CNc1cc(=O)n(-c2ccnc3c2cc(CN2CC=C(c4c(F)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)cc1F] |
| | 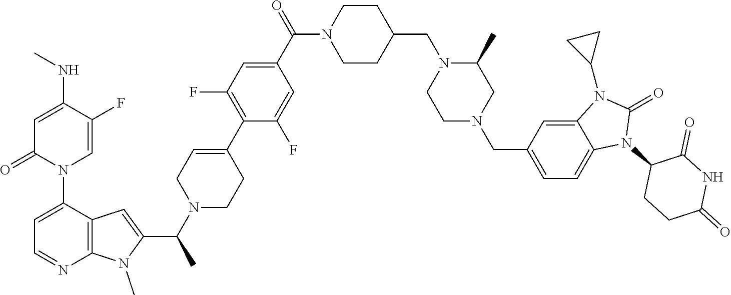 [SMILES: CNc1cc(=O)n(-c2ccnc3c2cc([C@H](C)N2CC=C(c4c(F)cc(C(=O)N5CCC(CN6CCN(Cc7ccc8c(c7)n(C7CC7)c(=O)n8[C@@H]7CCC(=O)NC7=O)C[C@@H]6C)CC5)cc4F)CC2)n3C)cc1F] |
| 3u13 | 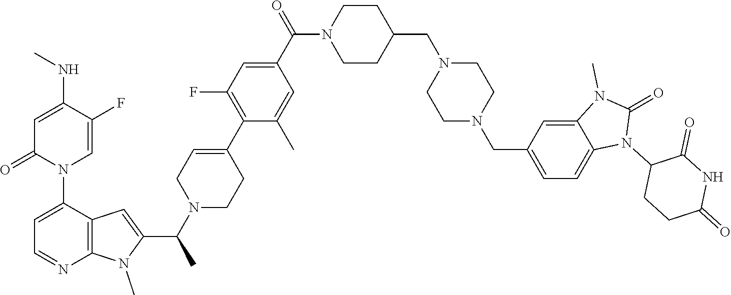 [SMILES: CNc1cc(=O)n(-c2ccnc3c2cc([C@H](C)N2CC=C(c4c(C)cc(C(=O)N5CCC(CN6CCN(Cc7ccc8c(c7)n(C)c(=O)n8C7CCC(=O)NC7=O)CC6)CC5)cc4F)CC2)n3C)cc1F] |
| 3u131 | 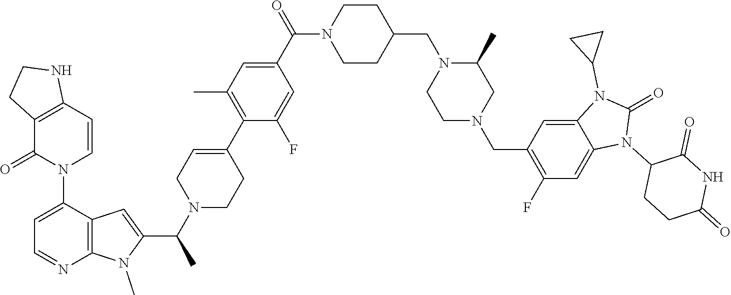 [SMILES: Cc1cc(C(=O)N2CCC(CN3CCN(Cc4cc5c(cc4F)n(C4CCC(=O)NC4=O)c(=O)n5C4CC4)C[C@@H]3C)CC2)cc(F)c1C1=CCN([C@@H](C)c2cc3c(-n4ccc5c(c4=O)CCN5)ccnc3n2C)CC1] |
| 3u132 and 3u133 | 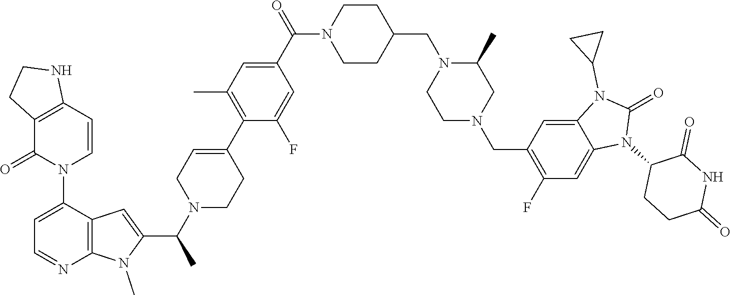 [SMILES: Cc1cc(C(=O)N2CCC(CN3CCN(Cc4cc5c(cc4F)n([C@H]4CCC(=O)NC4=O)c(=O)n5C4CC4)C[C@@H]3C)CC2)cc(F)c1C1=CCN([C@@H](C)c2cc3c(-n4ccc5c(c4=O)CCN5)ccnc3n2C)CC1] |
| | 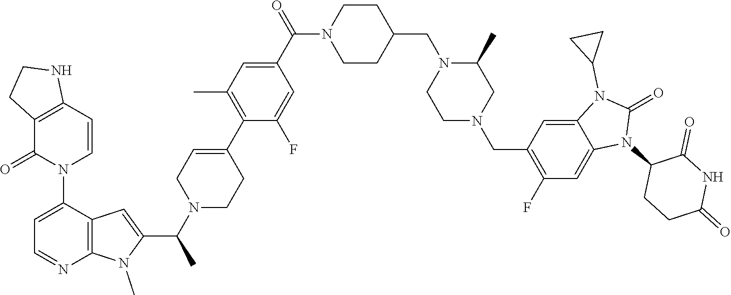 [SMILES: Cc1cc(C(=O)N2CCC(CN3CCN(Cc4cc5c(cc4F)n([C@@H]4CCC(=O)NC4=O)c(=O)n5C4CC4)C[C@@H]3C)CC2)cc(F)c1C1=CCN([C@@H](C)c2cc3c(-n4ccc5c(c4=O)CCN5)ccnc3n2C)CC1] |
| 3u134 | 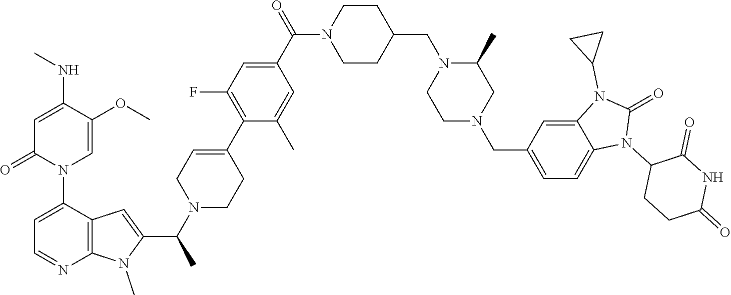 [SMILES: CNc1cc(=O)n(-c2ccnc3c2cc([C@H](C)N2CC=C(c4c(C)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)cc1OC] |
| 3u135 | 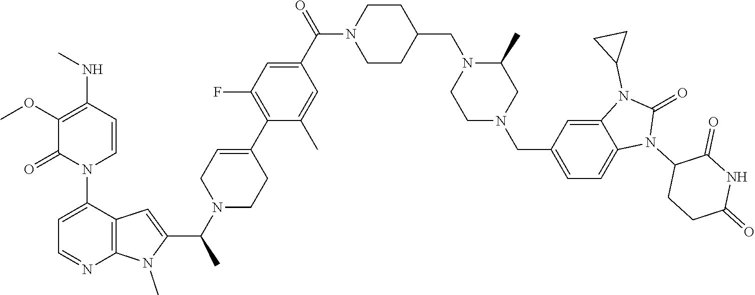 [SMILES: CNc1ccn(-c2ccnc3c2cc([C@H](C)N2CC=C(c4c(C)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)c(=O)c1OC] |
| 3u136 | 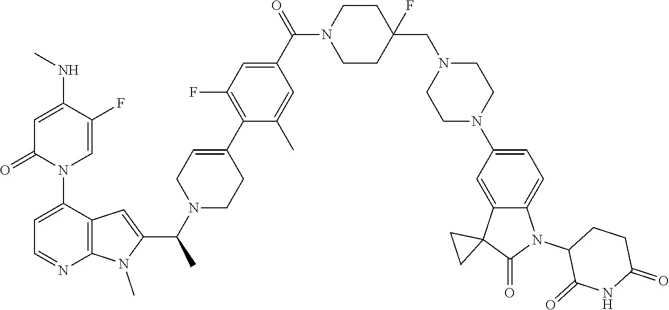 [SMILES: CNc1cc(=O)n(-c2ccnc3c2cc([C@H](C)N2CC=C(c4c(C)cc(C(=O)N5CCC(F)(CN6CCN(c7ccc8c(c7)C7(CC7)C(=O)N8C7CCC(=O)NC7=O)CC6)CC5)cc4F)CC2)n3C)cc1F] |
| 3u137 | 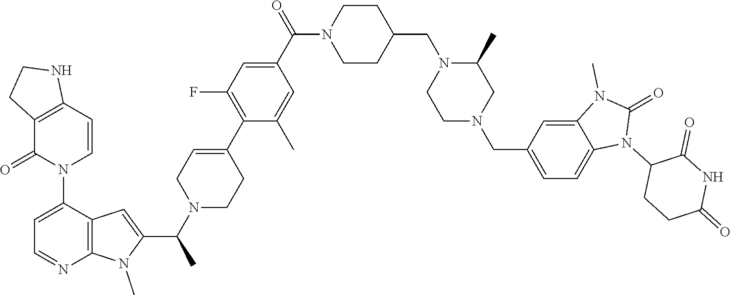 [SMILES: Cc1cc(C(=O)N2CCC(CN3CCN(Cc4ccc5c(c4)n(C)c(=O)n5C4CCC(=O)NC4=O)C[C@@H]3C)CC2)cc(F)c1C1=CCN([C@@H](C)c2cc3c(-n4ccc5c(c4=O)CCN5)ccnc3n2C)CC1] |
| 3u138 and 3u139 | 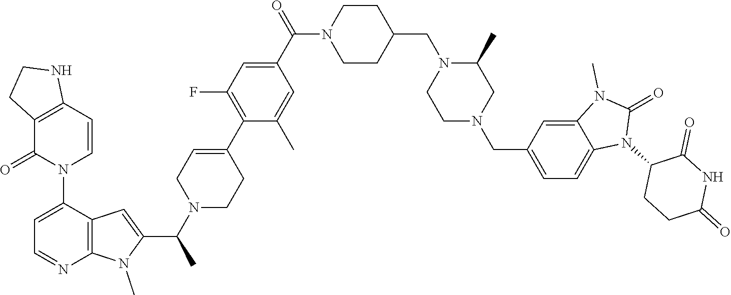 [SMILES: Cc1cc(C(=O)N2CCC(CN3CCN(Cc4ccc5c(c4)n(C)c(=O)n5[C@H]4CCC(=O)NC4=O)C[C@@H]3C)CC2)cc(F)c1C1=CCN([C@@H](C)c2cc3c(-n4ccc5c(c4=O)CCN5)ccnc3n2C)CC1] |
| | 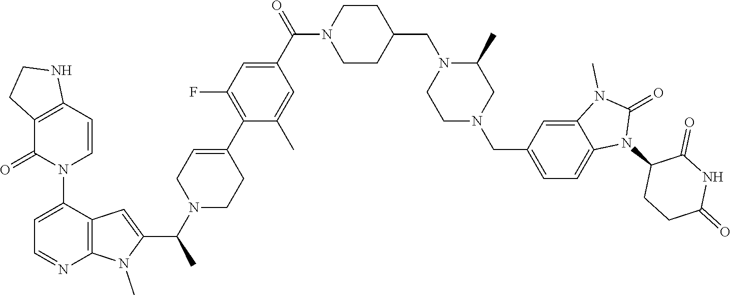 [SMILES: Cc1cc(C(=O)N2CCC(CN3CCN(Cc4ccc5c(c4)n(C)c(=O)n5[C@@H]4CCC(=O)NC4=O)C[C@@H]3C)CC2)cc(F)c1C1=CCN([C@@H](C)c2cc3c(-n4ccc5c(c4=O)CCN5)ccnc3n2C)CC1] |
| 3u14 and 3u15 | 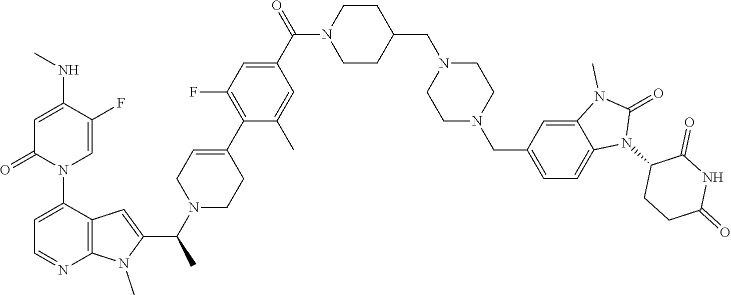 [SMILES: CNc1cc(=O)n(-c2ccnc3c2cc([C@H](C)N2CC=C(c4c(C)cc(C(=O)N5CCC(CN6CCN(Cc7ccc8c(c7)n(C)c(=O)n8[C@H]7CCC(=O)NC7=O)CC6)CC5)cc4F)CC2)n3C)cc1F] |
| | 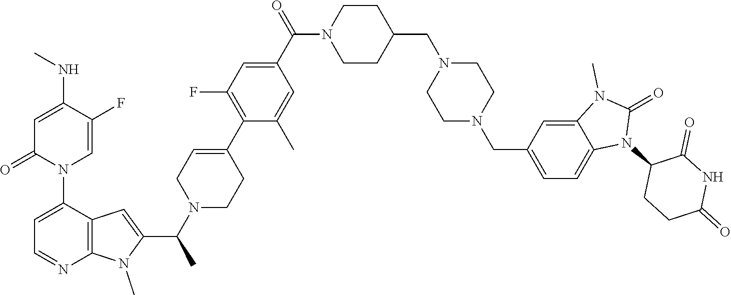 [SMILES: CNc1cc(=O)n(-c2ccnc3c2cc([C@H](C)N2CC=C(c4c(C)cc(C(=O)N5CCC(CN6CCN(Cc7ccc8c(c7)n(C)c(=O)n8[C@@H]7CCC(=O)NC7=O)CC6)CC5)cc4F)CC2)n3C)cc1F] |
| 3u140 | 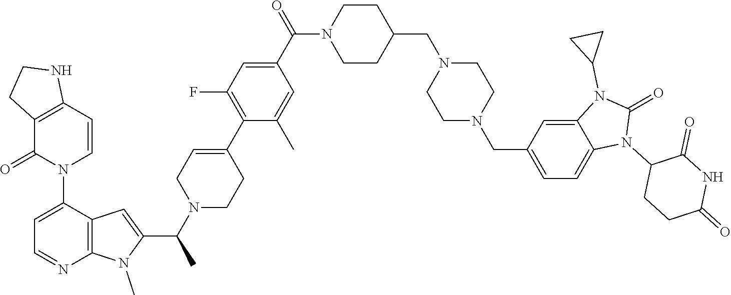 [SMILES: Cc1cc(C(=O)N2CCC(CN3CCN(Cc4ccc5c(c4)n(C4CC4)c(=O)n5C4CCC(=O)NC4=O)CC3)CC2)cc(F)c1C1=CCN([C@@H](C)c2cc3c(-n4ccc5c(c4=O)CCN5)ccnc3n2C)CC1] |
| 3u141 and 3u142 | 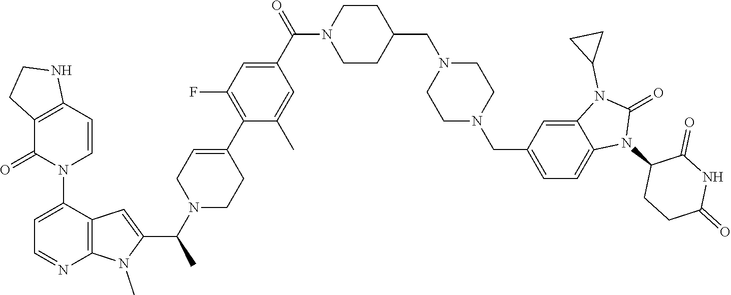 [SMILES: Cc1cc(C(=O)N2CCC(CN3CCN(Cc4ccc5c(c4)n(C4CC4)c(=O)n5[C@@H]4CCC(=O)NC4=O)CC3)CC2)cc(F)c1C1=CCN([C@@H](C)c2cc3c(-n4ccc5c(c4=O)CCN5)ccnc3n2C)CC1] |
| | 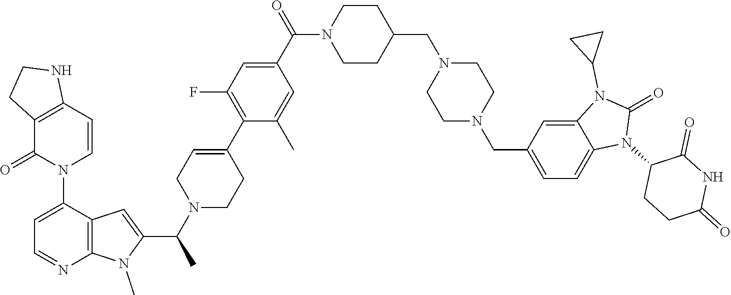 [SMILES: Cc1cc(C(=O)N2CCC(CN3CCN(Cc4ccc5c(c4)n(C4CC4)c(=O)n5[C@H]4CCC(=O)NC4=O)CC3)CC2)cc(F)c1C1=CCN([C@@H](C)c2cc3c(-n4ccc5c(c4=O)CCN5)ccnc3n2C)CC1] |
| 3u143 | 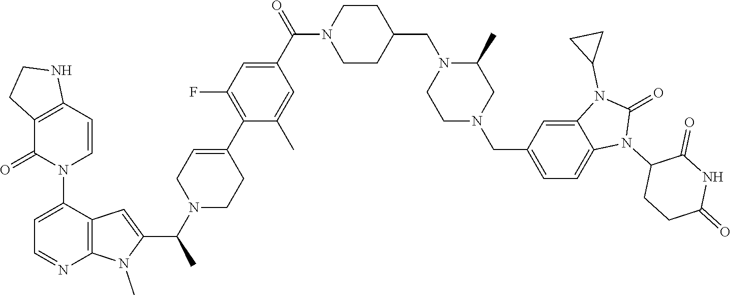 [SMILES: Cc1cc(C(=O)N2CCC(CN3CCN(Cc4ccc5c(c4)n(C4CC4)c(=O)n5C4CCC(=O)NC4=O)C[C@@H]3C)CC2)cc(F)c1C1=CCN([C@@H](C)c2cc3c(-n4ccc5c(c4=O)CCN5)ccnc3n2C)CC1] |
| 3u144 and 3u145 | 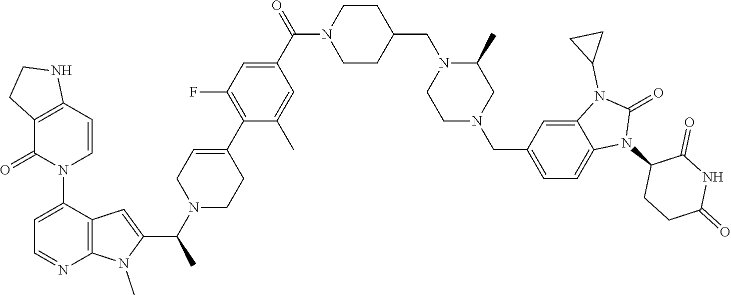 [SMILES: Cc1cc(C(=O)N2CCC(CN3CCN(Cc4ccc5c(c4)n(C4CC4)c(=O)n5[C@@H]4CCC(=O)NC4=O)C[C@@H]3C)CC2)cc(F)c1C1=CCN([C@@H](C)c2cc3c(-n4ccc5c(c4=O)CCN5)ccnc3n2C)CC1] |
| | 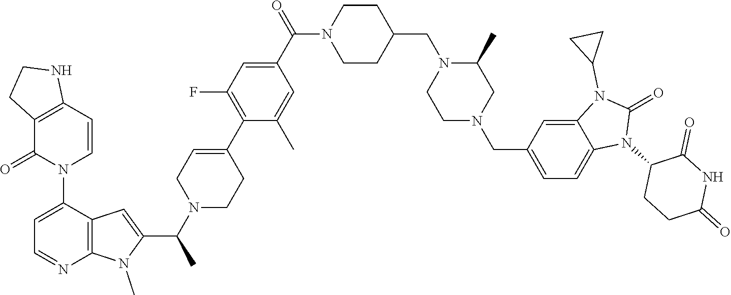 [SMILES: Cc1cc(C(=O)N2CCC(CN3CCN(Cc4ccc5c(c4)n(C4CC4)c(=O)n5[C@H]4CCC(=O)NC4=O)C[C@@H]3C)CC2)cc(F)c1C1=CCN([C@@H](C)c2cc3c(-n4ccc5c(c4=O)CCN5)ccnc3n2C)CC1] |
| 3u146 | 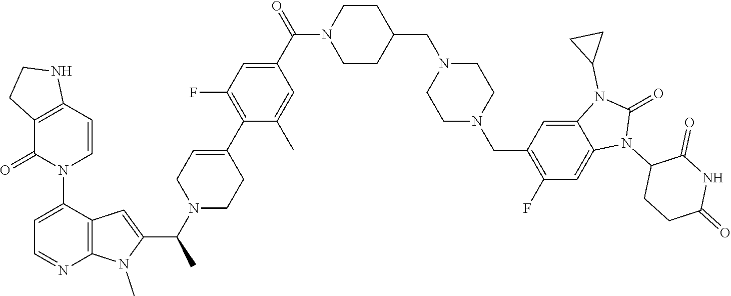 [SMILES: Cc1cc(C(=O)N2CCC(CN3CCN(Cc4cc5c(cc4F)n(C4CCC(=O)NC4=O)c(=O)n5C4CC4)CC3)CC2)cc(F)c1C1=CCN([C@@H](C)c2cc3c(-n4ccc5c(c4=O)CCN5)ccnc3n2C)CC1] |
| 3u147 and 3u148 | 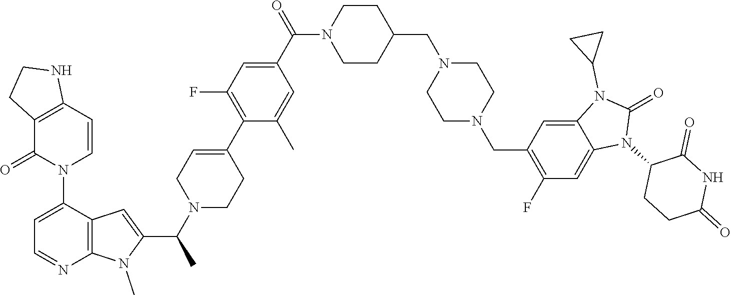 [SMILES: Cc1cc(C(=O)N2CCC(CN3CCN(Cc4cc5c(cc4F)n([C@H]4CCC(=O)NC4=O)c(=O)n5C4CC4)CC3)CC2)cc(F)c1C1=CCN([C@@H](C)c2cc3c(-n4ccc5c(c4=O)CCN5)ccnc3n2C)CC1] |
| | 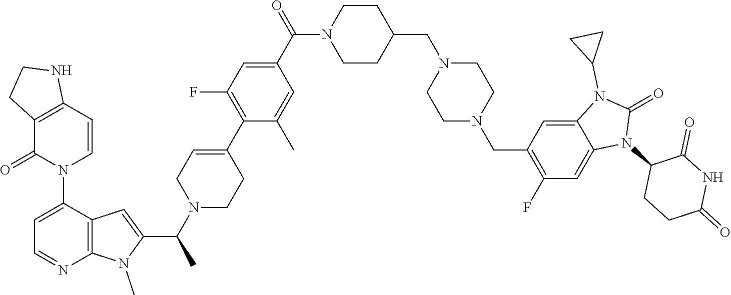 [SMILES: Cc1cc(C(=O)N2CCC(CN3CCN(Cc4cc5c(cc4F)n([C@@H]4CCC(=O)NC4=O)c(=O)n5C4CC4)CC3)CC2)cc(F)c1C1=CCN([C@@H](C)c2cc3c(-n4ccc5c(c4=O)CCN5)ccnc3n2C)CC1] |
| 3u149 | 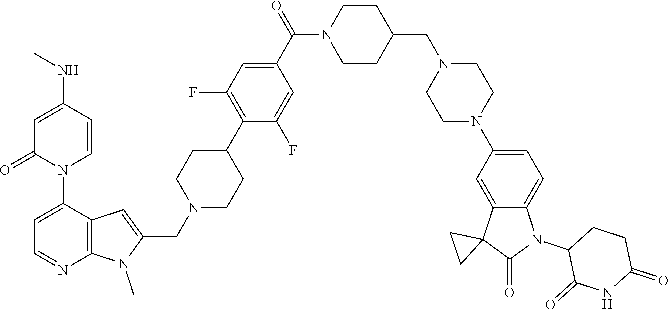 [SMILES: CNc1ccn(-c2ccnc3c2cc(CN2CCC(c4c(F)cc(C(=O)N5CCC(CN6CCN(c7ccc8c(c7)C7(CC7)C(=O)N8C7CCC(=O)NC7=O)CC6)CC5)cc4F)CC2)n3C)c(=O)c1] |
| 3u150 and 3u151 | 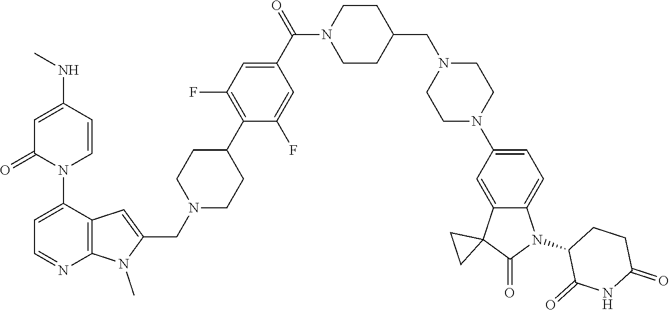 [SMILES: CNc1ccn(-c2ccnc3c2cc(CN2CCC(c4c(F)cc(C(=O)N5CCC(CN6CCN(c7ccc8c(c7)C7(CC7)C(=O)N8[C@@H]7CCC(=O)NC7=O)CC6)CC5)cc4F)CC2)n3C)c(=O)c1] |
| | 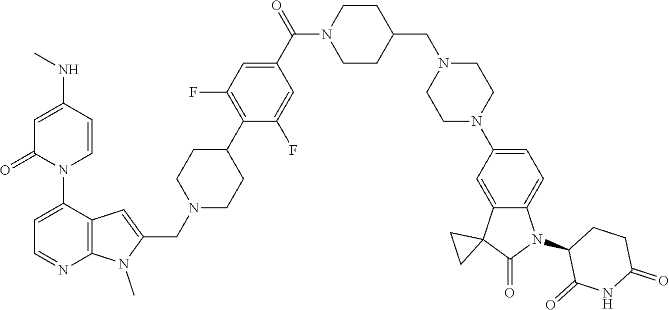 [SMILES: CNc1ccn(-c2ccnc3c2cc(CN2CCC(c4c(F)cc(C(=O)N5CCC(CN6CCN(c7ccc8c(c7)C7(CC7)C(=O)N8[C@H]7CCC(=O)NC7=O)CC6)CC5)cc4F)CC2)n3C)c(=O)c1] |
| 3u152 | 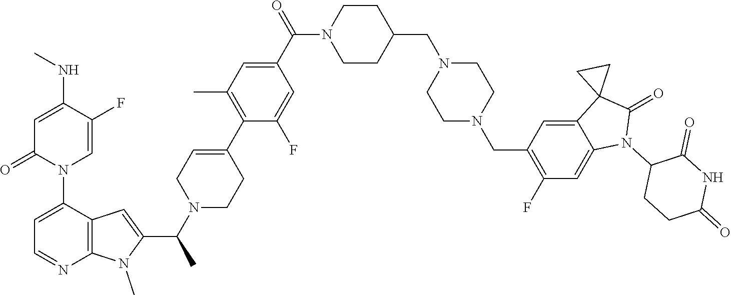 [SMILES: CNc1cc(=O)n(-c2ccnc3c2cc([C@H](C)N2CC=C(c4c(C)cc(C(=O)N5CCC(CN6CCN(Cc7cc8c(cc7F)N(C7CCC(=O)NC7=O)C(=O)C87CC7)CC6)CC5)cc4F)CC2)n3C)cc1F] |
| 3u153 and 3u154 | 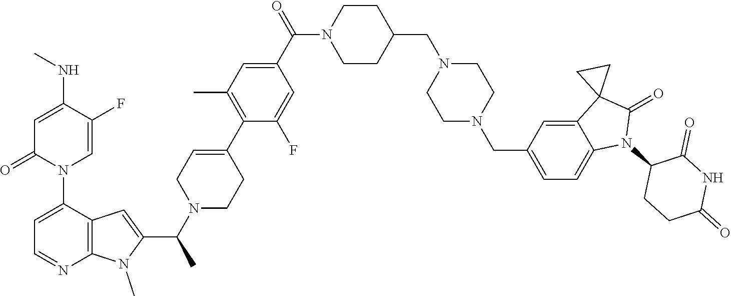 [SMILES: CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8[C@@H]7CCC(=O)NC7=O)CC6)CC5)cc4F)CC2)n3C)cc1F] |
| | 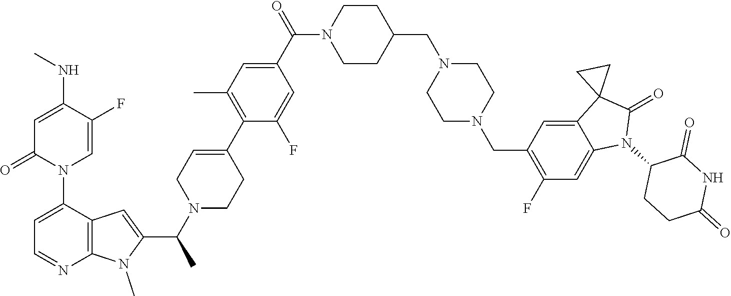 [SMILES: CNc1cc(=O)n(-c2ccnc3c2cc([C@H](C)N2CC=C(c4c(C)cc(C(=O)N5CCC(CN6CCN(Cc7cc8c(cc7F)N([C@H]7CCC(=O)NC7=O)C(=O)C87CC7)CC6)CC5)cc4F)CC2)n3C)cc1F] |
| 3u155 | 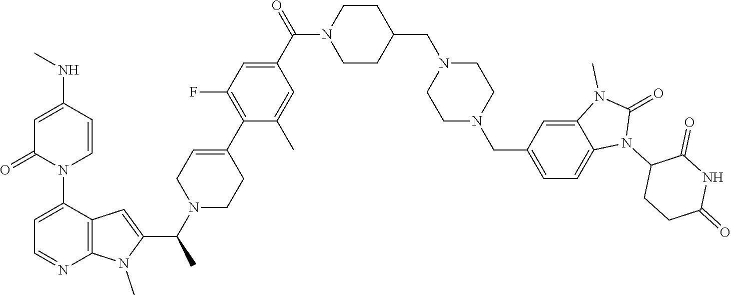 [SMILES: CNc1ccn(-c2ccnc3c2cc([C@H](C)N2CC=C(c4c(C)cc(C(=O)N5CCC(CN6CCN(Cc7ccc8c(c7)n(C)c(=O)n8C7CCC(=O)NC7=O)CC6)CC5)cc4F)CC2)n3C)c(=O)c1] |
| 3u156 and 3u157 | 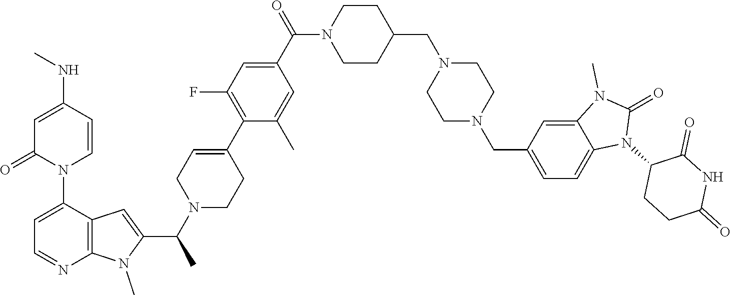 [SMILES: CNc1ccn(-c2ccnc3c2cc([C@H](C)N2CC=C(c4c(C)cc(C(=O)N5CCC(CN6CCN(Cc7ccc8c(c7)n(C)c(=O)n8[C@H]7CCC(=O)NC7=O)CC6)CC5)cc4F)CC2)n3C)c(=O)c1] |
| | 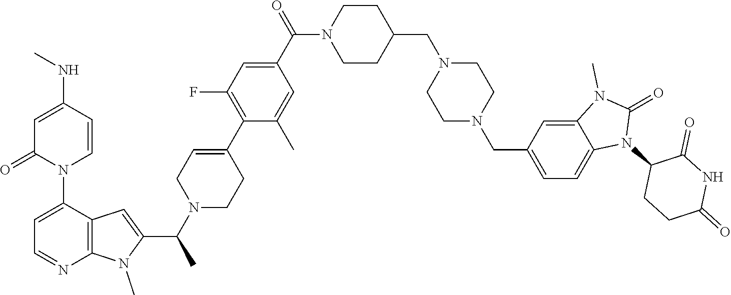 [SMILES: CNc1ccn(-c2ccnc3c2cc([C@H](C)N2CC=C(c4c(C)cc(C(=O)N5CCC(CN6CCN(Cc7ccc8c(c7)n(C)c(=O)n8[C@@H]7CCC(=O)NC7=O)CC6)CC5)cc4F)CC2)n3C)c(=O)c1] |
| 3u158 | 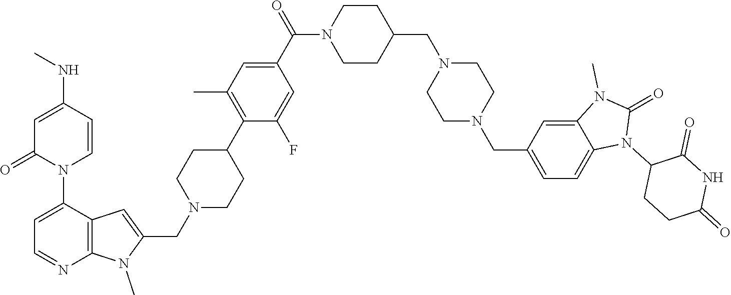 [SMILES: CNc1ccn(-c2ccnc3c2cc(CN2CCC(c4c(C)cc(C(=O)N5CCC(CN6CCN(Cc7ccc8c(c7)n(C)c(=O)n8C7CCC(=O)NC7=O)CC6)CC5)cc4F)CC2)n3C)c(=O)c1] |
| 3u159 and 3u160 | 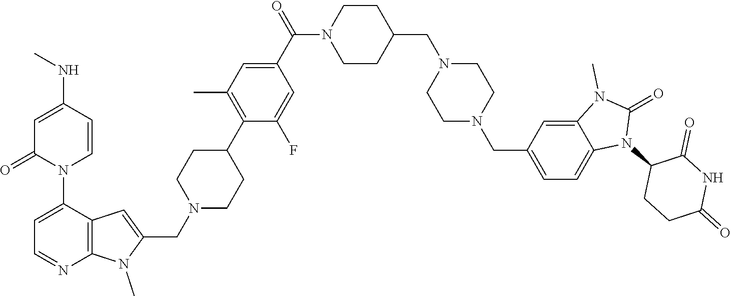 [SMILES: CNc1ccn(-c2ccnc3c2cc(CN2CCC(c4c(C)cc(C(=O)N5CCC(CN6CCN(Cc7ccc8c(c7)n(C)c(=O)n8[C@@H]7CCC(=O)NC7=O)CC6)CC5)cc4F)CC2)n3C)c(=O)c1] |
| | 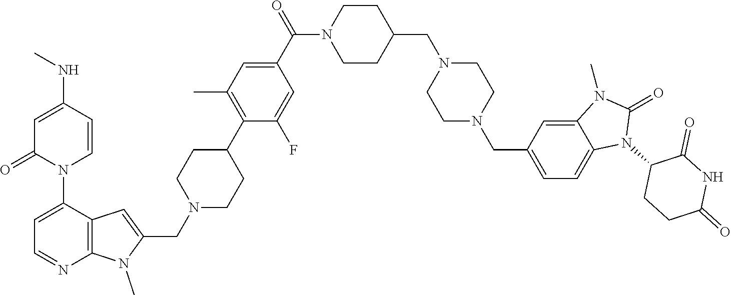 [SMILES: CNc1ccn(-c2ccnc3c2cc(CN2CCC(c4c(C)cc(C(=O)N5CCC(CN6CCN(Cc7ccc8c(c7)n(C)c(=O)n8[C@H]7CCC(=O)NC7=O)CC6)CC5)cc4F)CC2)n3C)c(=O)c1] |
| 3u16 | 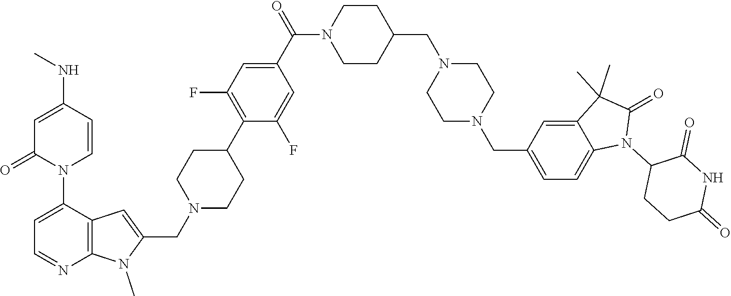 [SMILES: CNc1ccn(-c2ccnc3c2cc(CN2CCC(c4c(F)cc(C(=O)N5CCC(CN6CCN(Cc7ccc8c(c7)C(C)(C)C(=O)N8C7CCC(=O)NC7=O)CC6)CC5)cc4F)CC2)n3C)c(=O)c1] |
| 3u161 and 3u162 | 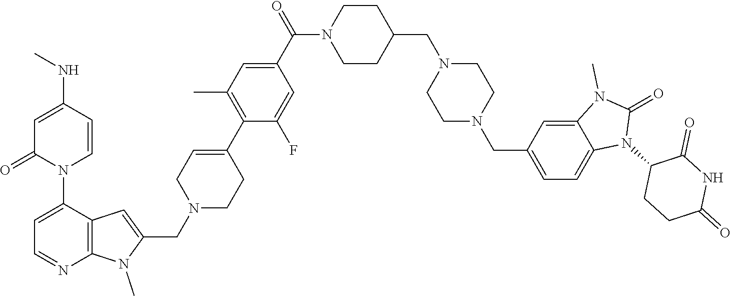 [SMILES: CNc1ccn(-c2ccnc3c2cc(CN2CC=C(c4c(C)cc(C(=O)N5CCC(CN6CCN(Cc7ccc8c(c7)n(C)c(=O)n8[C@H]7CCC(=O)NC7=O)CC6)CC5)cc4F)CC2)n3C)c(=O)c1] |
| | 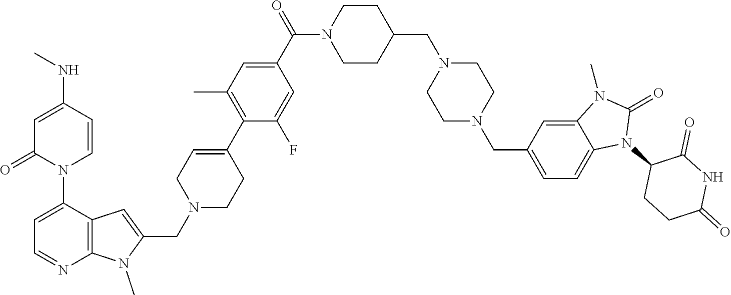 [SMILES: CNc1ccn(-c2ccnc3c2cc(CN2CC=C(c4c(C)cc(C(=O)N5CCC(CN6CCN(Cc7ccc8c(c7)n(C)c(=O)n8[C@@H]7CCC(=O)NC7=O)CC6)CC5)cc4F)CC2)n3C)c(=O)c1] |
| 3u163 | 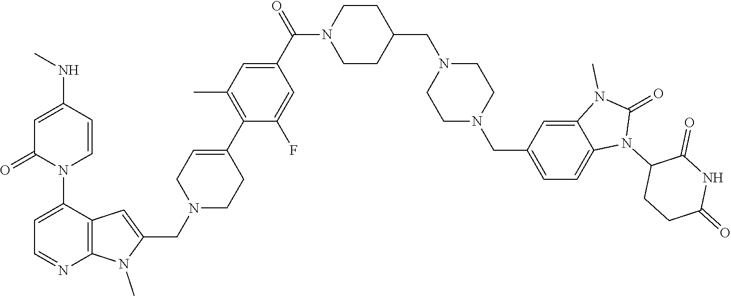 [SMILES: CNc1ccn(-c2ccnc3c2cc(CN2CC=C(c4c(C)cc(C(=O)N5CCC(CN6CCN(Cc7ccc8c(c7)n(C)c(=O)n8C7CCC(=O)NC7=O)CC6)CC5)cc4F)CC2)n3C)c(=O)c1] |
| 3u164 | 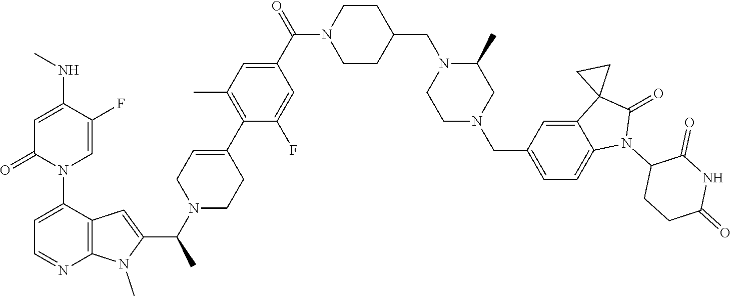 [SMILES: CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)cc1F] |
| 3u165 | 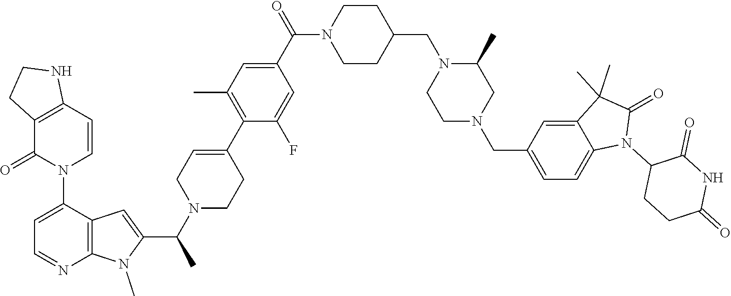 [SMILES: Cc1cc(C(=O)N2CCC(CN3CCN(Cc4ccc5c(c4)C(C)(C)C(=O)N5C4CCC(=O)NC4=O)C[C@@H]3C)CC2)cc(F)c1C1=CCN([C@@H](C)c2cc3c(-n4ccc5c(c4=O)CCN5)ccnc3n2C)CC1] |
| 3u166 | 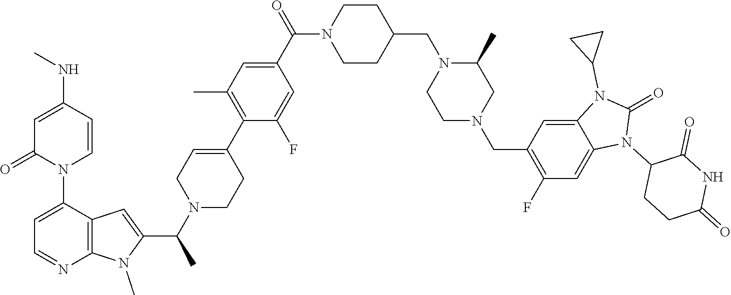 [SMILES: CNc1ccn(-c2ccnc3c2cc([C@H](C)N2CC=C(c4c(C)cc(C(=O)N5CCC(CN6CCN(Cc7cc8c(cc7F)n(C7CCC(=O)NC7=O)c(=O)n8C7CC7)C[C@@H]6C)CC5)cc4F)CC2)n3C)c(=O)c1] |
| 3u167 and 3u168 | 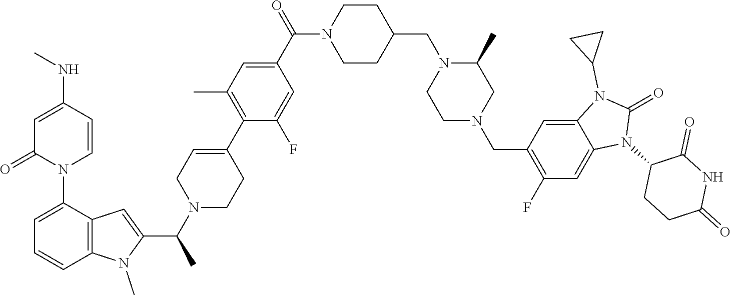 [SMILES: CNc1ccn(-c2cccc3c2cc([C@H](C)N2CC=C(c4c(C)cc(C(=O)N5CCC(CN6CCN(Cc7cc8c(cc7F)n([C@H]7CCC(=O)NC7=O)c(=O)n8C7CC7)C[C@@H]6C)CC5)cc4F)CC2)n3C)c(=O)c1] |
| | 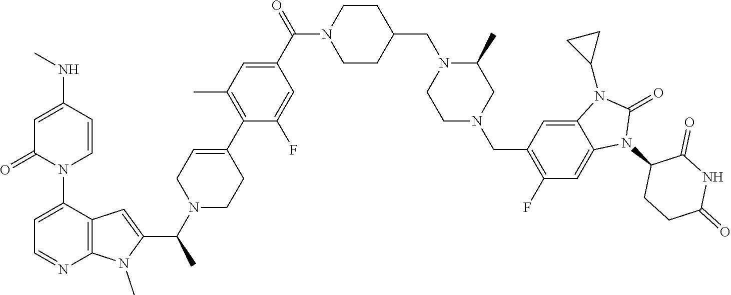 [SMILES: CNc1ccn(-c2ccnc3c2cc([C@H](C)N2CC=C(c4c(C)cc(C(=O)N5CCC(CN6CCN(Cc7cc8c(cc7F)n([C@@H]7CCC(=O)NC7=O)c(=O)n8C7CC7)C[C@@H]6C)CC5)cc4F)CC2)n3C)c(=O)c1] |
| 3u169 | 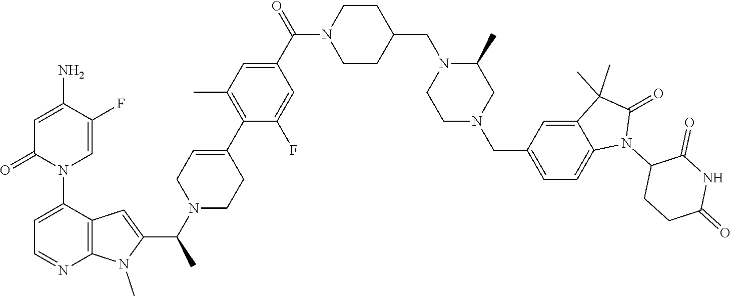 [SMILES: Cc1cc(C(=O)N2CCC(CN3CCN(Cc4ccc5c(c4)C(C)(C)C(=O)N5C4CCC(=O)NC4=O)C[C@@H]3C)CC2)cc(F)c1C1=CCN([C@@H](C)c2cc3c(-n4cc(F)c(N)cc4=O)ccnc3n2C)CC1] |
| 3u17 and 3u18 | 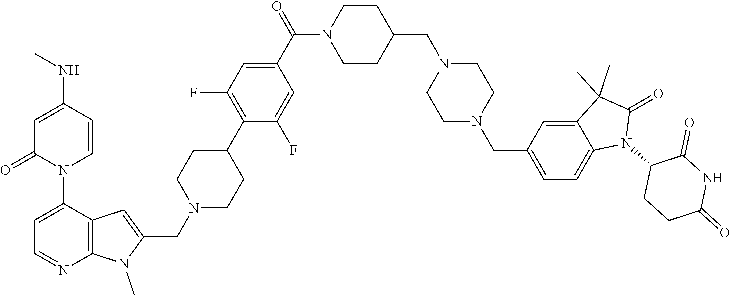 [SMILES: CNc1ccn(-c2ccnc3c2cc(CN2CCC(c4c(F)cc(C(=O)N5CCC(CN6CCN(Cc7ccc8c(c7)C(C)(C)C(=O)N8[C@H]7CCC(=O)NC7=O)CC6)CC5)cc4F)CC2)n3C)c(=O)c1] |
| | 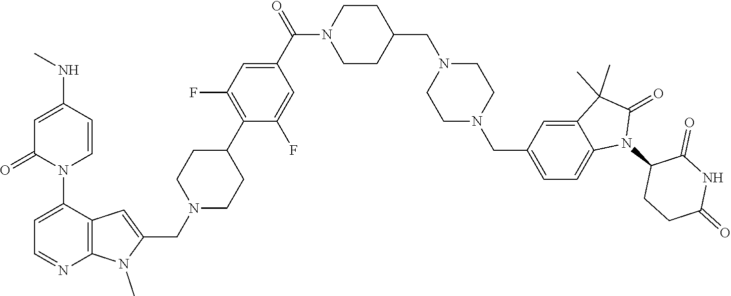 [SMILES: CNc1ccn(-c2ccnc3c2cc(CN2CCC(c4c(F)cc(C(=O)N5CCC(CN6CCN(Cc7ccc8c(c7)C(C)(C)C(=O)N8[C@@H]7CCC(=O)NC7=O)CC6)CC5)cc4F)CC2)n3C)c(=O)c1] |
| 3u170 and 3u171 | 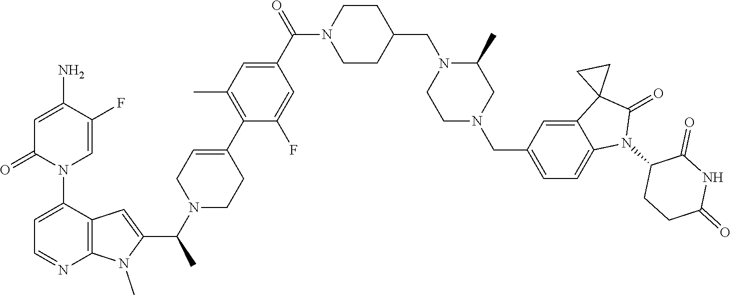 [SMILES: Cc1cc(C(=O)N2CCC(CN3CCN(Cc4ccc5c(c4)C4(CC4)C(=O)N5[C@H]4CCC(=O)NC4=O)C[C@@H]3C)CC2)cc(F)c1C1=CCN([C@@H](C)c2cc3c(-n4cc(F)c(N)cc4=O)ccnc3n2C)CC1] |
| | 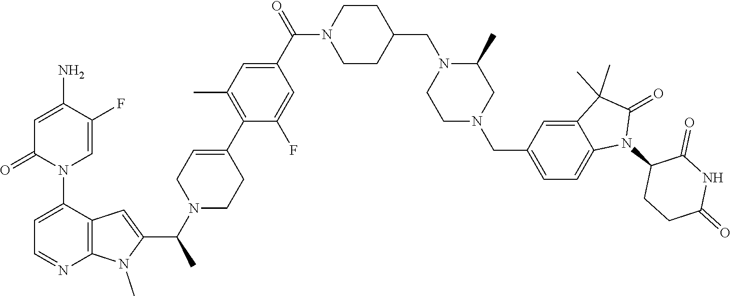 [SMILES: Cc1cc(C(=O)N2CCC(CN3CCN(Cc4ccc5c(c4)C(C)(C)C(=O)N5[C@@H]4CCC(=O)NC4=O)C[C@@H]3C)CC2)cc(F)c1C1=CCN([C@@H](C)c2cc3c(-n4cc(F)c(N)cc4=O)ccnc3n2C)CC1] |
| 3u172 | 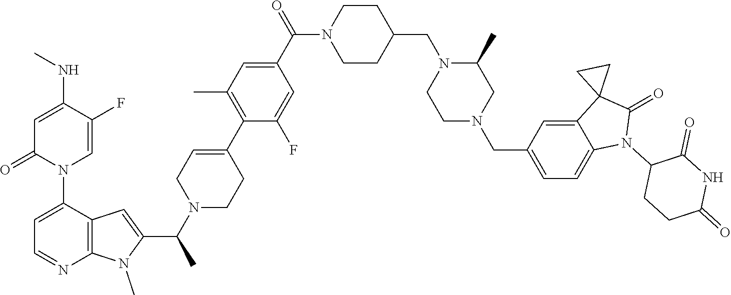 [SMILES: CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)cc1F] |
| 3u173 and 3u174 | 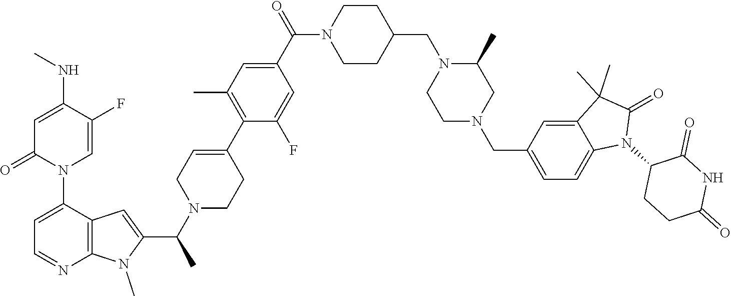 [SMILES: CNc1cc(=O)n(-c2ccnc3c2cc([C@H](C)N2CC=C(c4c(C)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)cc1F] |
| | 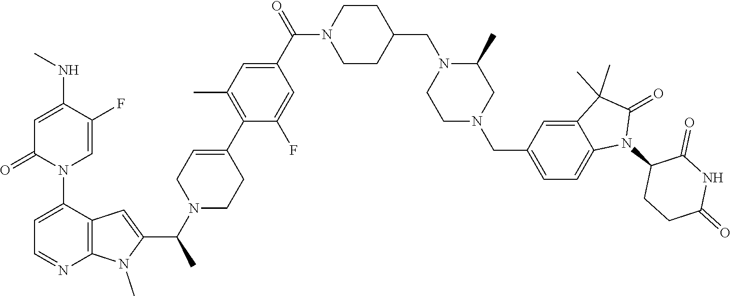 [SMILES: CNc1cc(=O)n(-c2ccnc3c2cc([C@H](C)N2CC=C(c4c(C)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)cc1F] |
| 3u175 | 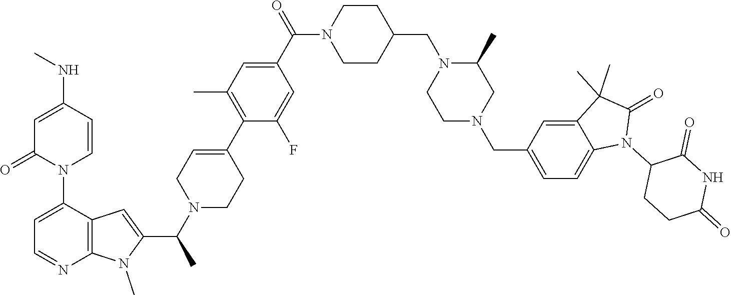 [SMILES: CNc1ccn(-c2ccnc3c2cc([C@H](C)N2CC=C(c4c(C)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)c(=O)c1] |
| 3u176 and 3u177 | 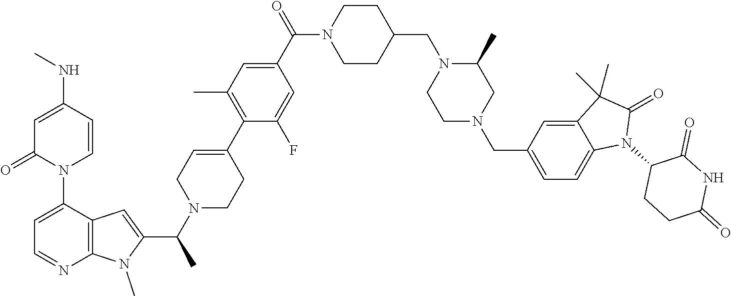 [SMILES: CNc1ccn(-c2ccnc3c2cc([C@H](C)N2CC=C(c4c(C)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)c(=O)c1] |
| | 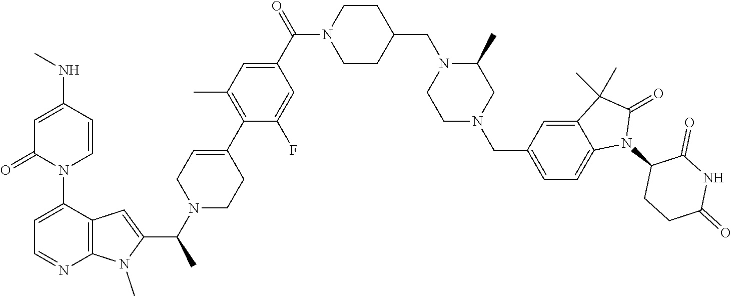 [SMILES: CNc1ccn(-c2ccnc3c2cc([C@H](C)N2CC=C(c4c(C)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)c(=O)c1] |
| 3u178 | 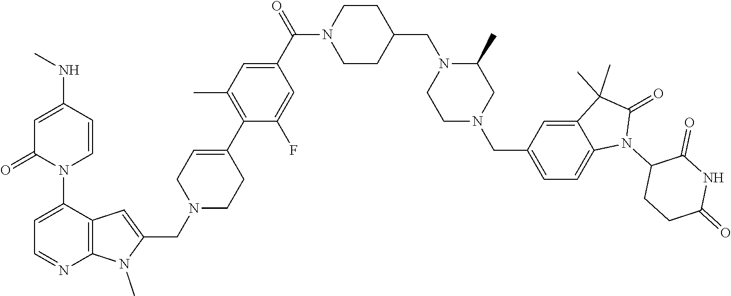 [SMILES: CNc1ccn(-c2ccnc3c2cc(CN2CC=C(c4c(C)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)c(=O)c1] |
| 3u179 and 3u180 | 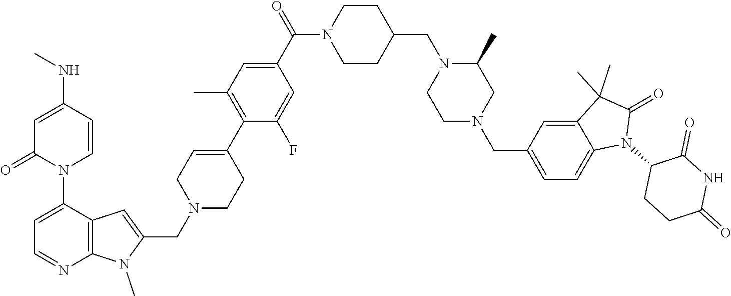 [SMILES: CNc1ccn(-c2ccnc3c2cc(CN2CC=C(c4c(C)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)c(=O)c1] |
| | 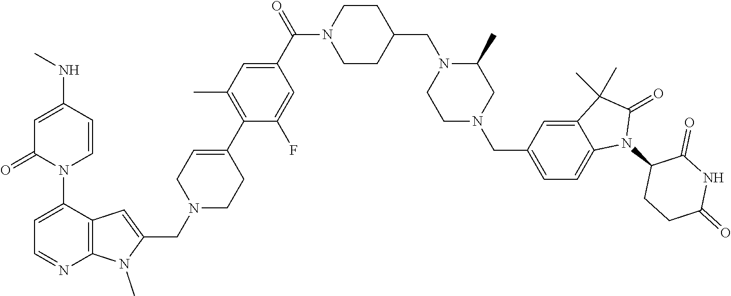 [SMILES: CNc1ccn(-c2ccnc3c2cc(CN2CC=C(c4c(C)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)c(=O)c1] |
| 3u181 | 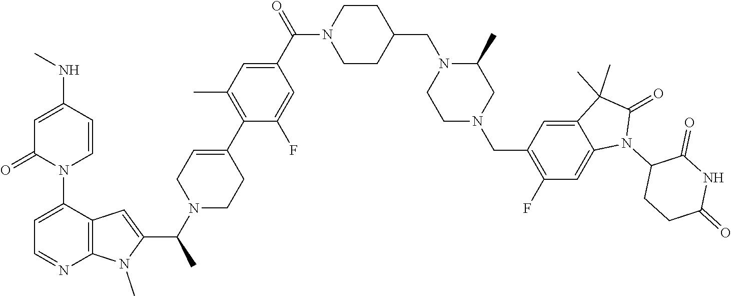 [SMILES: CNc1ccn(-c2ccnc3c2cc([C@H](C)N2CC=C(c4c(C)cc(C(=O)N5CCC(CN6CCN(Cc7cc8c(cc7F)N(C7CCC(=O)NC7=O)C(=O)C8(C)C)C[C@@H]6C)CC5)cc4F)CC2)n3C)c(=O)c1] |
| 3u182 and 3u183 | 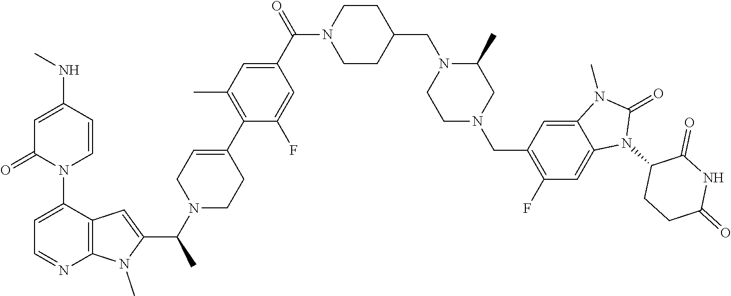 [SMILES: CNc1ccn(-c2ccnc3c2cc([C@H](C)N2CC=C(c4c(C)cc(C(=O)N5CCC(CN6CCN(Cc7cc8c(cc7F)n([C@H]7CCC(=O)NC7=O)c(=O)n8C)C[C@@H]6C)CC5)cc4F)CC2)n3C)c(=O)c1] |
| | 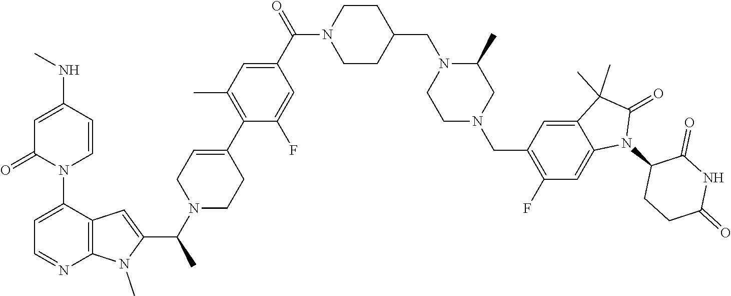 [SMILES: CNc1ccn(-c2ccnc3c2cc([C@H](C)N2CC=C(c4c(C)cc(C(=O)N5CCC(CN6CCN(Cc7cc8c(cc7F)N([C@@H]7CCC(=O)NC7=O)C(=O)C8(C)C)C[C@@H]6C)CC5)cc4F)CC2)n3C)c(=O)c1] |
| 3u184 | 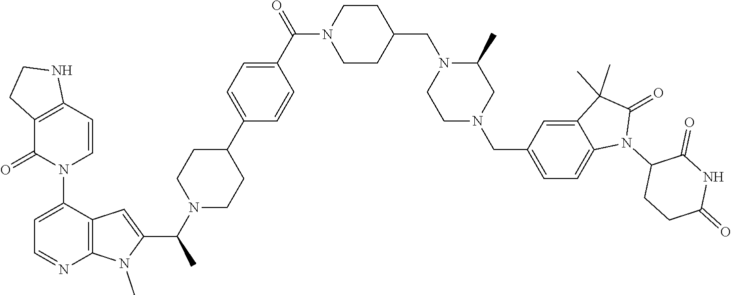 [SMILES: C[C@H]1CN(Cc2ccc3c(c2)C(C)(C)C(=O)N3C2CCC(=O)NC2=O)CCN1CC1CCN(C(=O)c2ccc(C3CCN([C@@H](C)c4cc5c(-n6ccc7c(c6=O)CCN7)ccnc5n4C)CC3)cc2)CC1] |
| 3u185 and 3u186 | 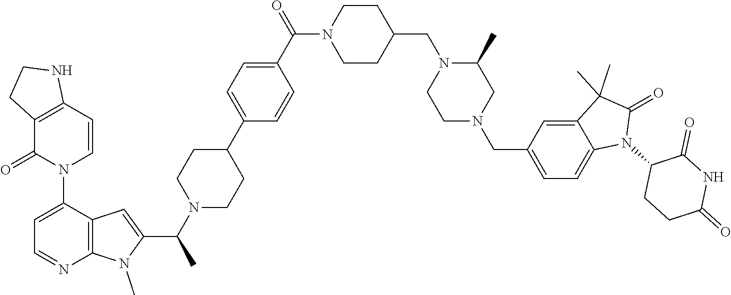 [SMILES: C[C@H]1CN(Cc2ccc3c(c2)C(C)(C)C(=O)N3[C@H]2CCC(=O)NC2=O)CCN1CC1CCN(C(=O)c2ccc(C3CCN([C@@H](C)c4cc5c(-n6ccc7c(c6=O)CCN7)ccnc5n4C)CC3)cc2)CC1] |
| | 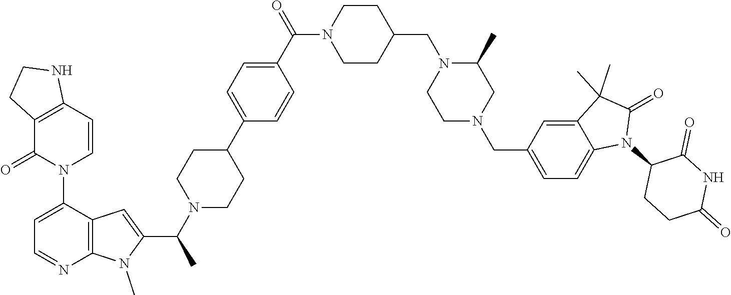 [SMILES: C[C@H]1CN(Cc2ccc3c(c2)C(C)(C)C(=O)N3[C@@H]2CCC(=O)NC2=O)CCN1CC1CCN(C(=O)c2ccc(C3CCN([C@@H](C)c4cc5c(-n6ccc7c(c6=O)CCN7)ccnc5n4C)CC3)cc2)CC1] |
| 3u187 | 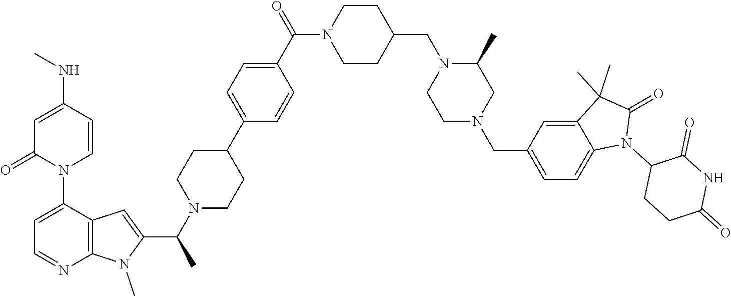 [SMILES: CNc1ccn(-c2ccnc3c2cc([C@H](C)N2CCC(c4ccc(C(=O)N5CCC(CN6CCN(Cc7ccc8c(c7)C(C)(C)C(=O)N8C7CCC(=O)NC7=O)C[C@@H]6C)CC5)cc4)CC2)n3C)c(=O)c1] |
| 3u188 and 3u189 | 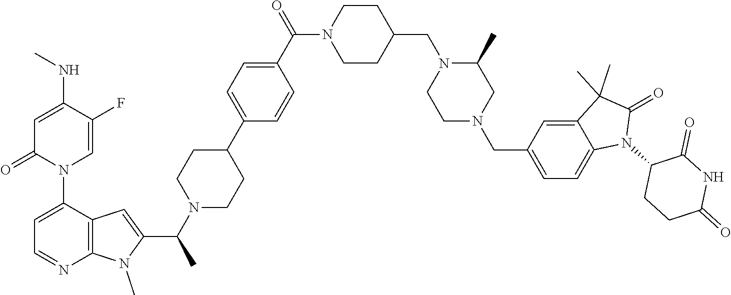 [SMILES: CNc1cc(=O)n(-c2ccnc3c2cc([C@H](C)N2CCC(c4ccc(C(=O)N5CCC(CN6CCN(Cc7ccc8c(c7)C(C)(C)C(=O)N8[C@H]7CCC(=O)NC7=O)C[C@@H]6C)CC5)cc4)CC2)n3C)cc1F] |
| | 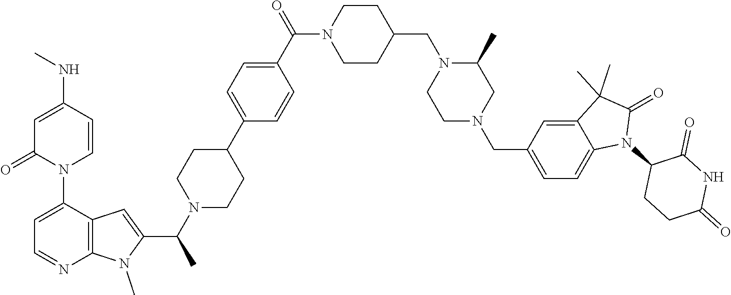 [SMILES: CNc1ccn(-c2ccnc3c2cc([C@H](C)N2CCC(c4ccc(C(=O)N5CCC(CN6CCN(Cc7ccc8c(c7)C(C)(C)C(=O)N8[C@@H]7CCC(=O)NC7=O)C[C@@H]6C)CC5)cc4)CC2)n3C)c(=O)c1] |
| 3u19 | 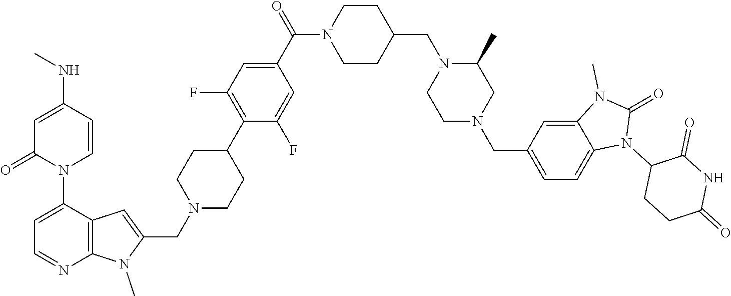 [SMILES: CNc1ccn(-c2ccnc3c2cc(CN2CCC(c4c(F)cc(C(=O)N5CCC(CN6CCN(Cc7ccc8c(c7)n(C)c(=O)n8C7CCC(=O)NC7=O)C[C@@H]6C)CC5)cc4F)CC2)n3C)c(=O)c1] |
| 3u190 | 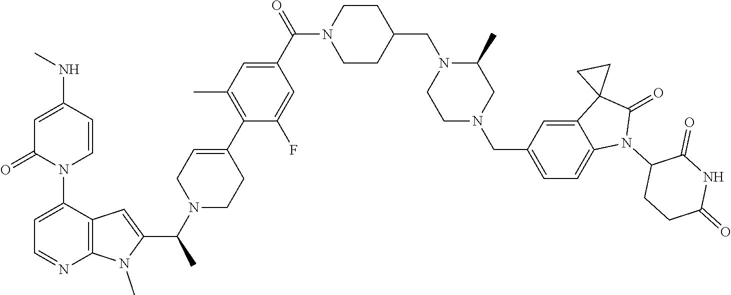 [SMILES: CNc1ccn(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)c(=O)c1] |
| 3u191 and 3u192 | 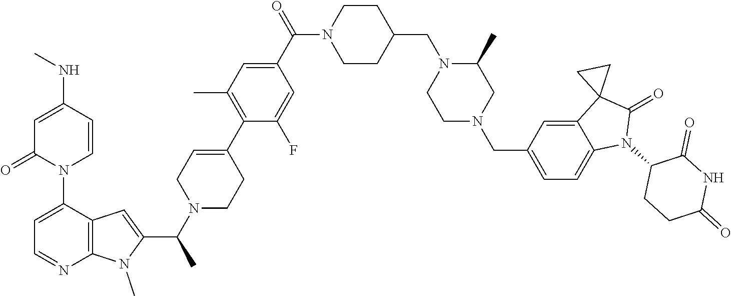 [SMILES: CNc1ccn(-c2ccnc3c2cc([C@H](C)N2CC=C(c4c(C)cc(C(=O)N5CCC(CN6CCN(Cc7ccc8c(c7)C7(CC7)C(=O)N8[C@H]7CCC(=O)NC7=O)C[C@@H]6C)CC5)cc4F)CC2)n3C)c(=O)c1] |
| | 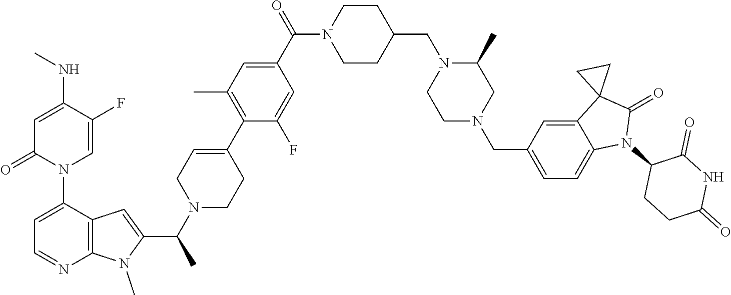 [SMILES: CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8[C@@H]7CCC(=O)NC7=O)C[C@@H]6C)CC5)cc4F)CC2)n3C)cc1F] |
| 3u193 | 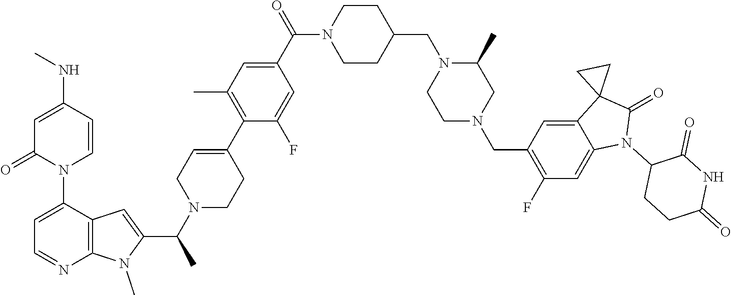 [SMILES: CNc1ccn(-c2ccnc3c2cc([C@H](C)N2CC=C(c4c(C)cc(C(=O)N5CCC(CN6CCN(Cc7cc8c(cc7F)N(C7CCC(=O)NC7=O)C(=O)C87CC7)C[C@@H]6C)CC5)cc4F)CC2)n3C)c(=O)c1] |
| 3u194 and 3u195 | 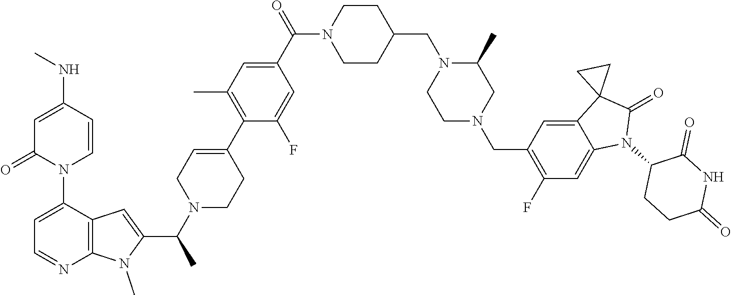 [SMILES: CNc1ccn(-c2ccnc3c2cc([C@H](C)N2CC=C(c4c(C)cc(C(=O)N5CCC(CN6CCN(Cc7cc8c(cc7F)N([C@H]7CCC(=O)NC7=O)C(=O)C87CC7)C[C@@H]6C)CC5)cc4F)CC2)n3C)c(=O)c1] |
| | 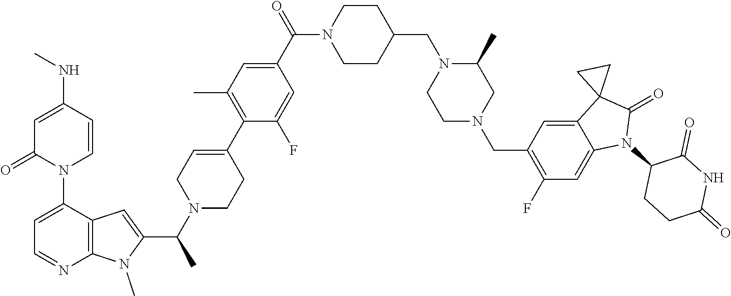 [SMILES: CNc1ccn(-c2ccnc3c2cc([C@H](C)N2CC=C(c4c(C)cc(C(=O)N5CCC(CN6CCN(Cc7cc8c(cc7F)N([C@@H]7CCC(=O)NC7=O)C(=O)C87CC7)C[C@@H]6C)CC5)cc4F)CC2)n3C)c(=O)c1] |
| 3u196 | 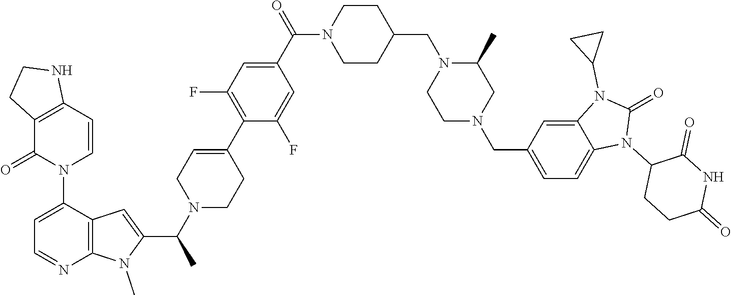 [SMILES: C[C@H]1CN(Cc2ccc3c(c2)n(C2CC2)c(=O)n3C2CCC(=O)NC2=O)CCN1CC1CCN(C(=O)c2cc(F)c(C3=CCN([C@@H](C)c4cc5c(-n6ccc7c(c6=O)CCN7)ccnc5n4C)CC3)c(F)c2)CC1] |
| 3u197 | 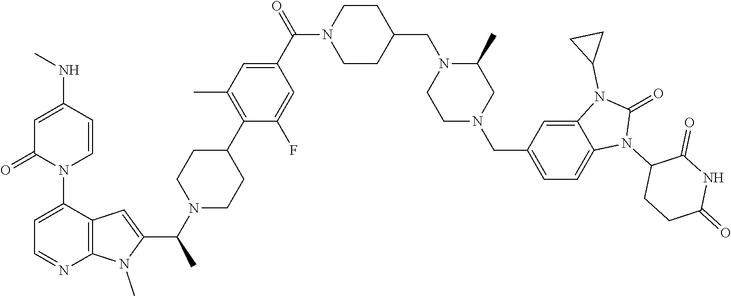 [SMILES: CNc1ccn(-c2ccnc3c2cc([C@H](C)N2CCC(c4c(C)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)c(=O)c1] |
| 3u198 and 3u199 | 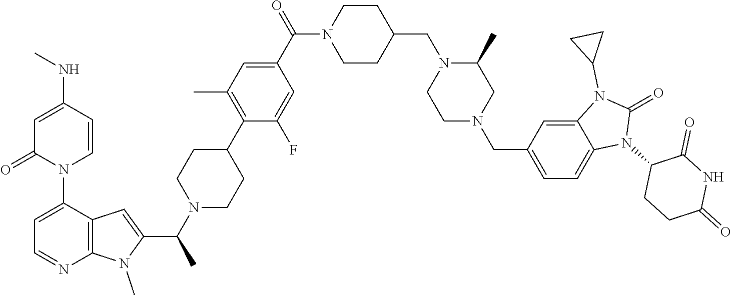 [SMILES: CNc1ccn(-c2ccnc3c2cc([C@H](C)N2CCC(c4c(C)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)c(=O)c1] |
| | 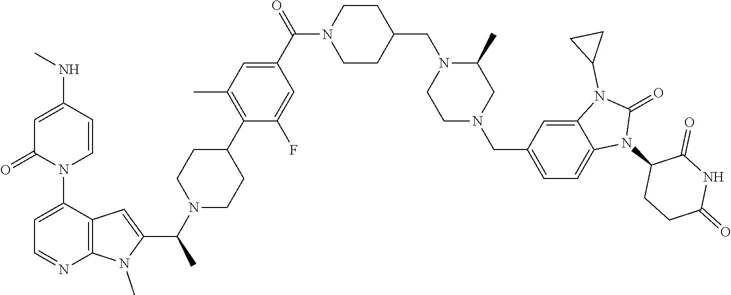 [SMILES: CNc1ccn(-c2ccnc3c2cc([C@H](C)N2CCC(c4c(C)cc(C(=O)N5CCC(CN6CCN(Cc7ccc8c(c7)n(C7CC7)c(=O)n8[C@@H]7CCC(=O)NC7=O)C[C@@H]6C)CC5)cc4F)CC2)n3C)c(=O)c1] |
| 3u2 and 3u3 | 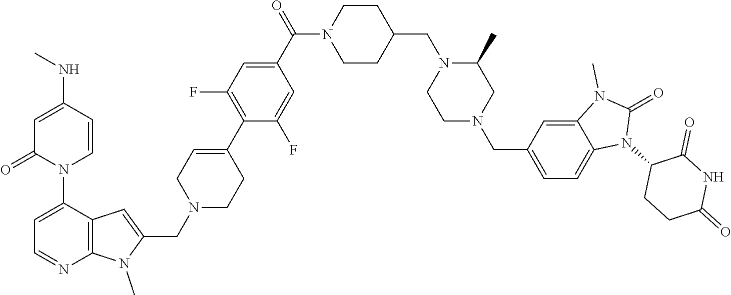 [SMILES: CNc1ccn(-c2ccnc3c2cc(CN2CC=C(c4c(F)cc(C(=O)N5CCC(CN6CCN(Cc7ccc8c(c7)n(C)c(=O)n8[C@H]7CCC(=O)NC7=O)C[C@@H]6C)CC5)cc4F)CC2)n3C)c(=O)c1] |
| | 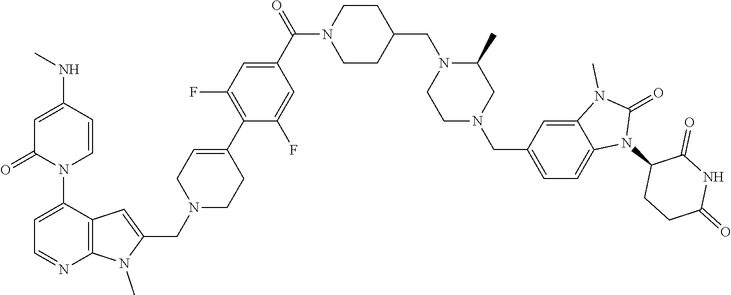 [SMILES: CNc1ccn(-c2ccnc3c2cc(CN2CC=C(c4c(F)cc(C(=O)N5CCC(CN6CCN(Cc7ccc8c(c7)n(C)c(=O)n8[C@@H]7CCC(=O)NC7=O)C[C@@H]6C)CC5)cc4F)CC2)n3C)c(=O)c1] |
| 3u20 and 3u21 | 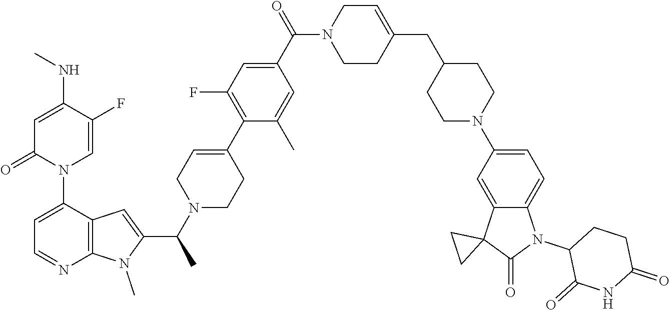 [SMILES: CNc1cc(=O)n(-c2ccnc3c2cc([C@H](C)N2CC=C(c4c(C)cc(C(=O)N5CC=C(CC6CCN(c7ccc8c(c7)C7(CC7)C(=O)N8C7CCC(=O)NC7=O)CC6)CC5)cc4F)CC2)n3C)cc1F] |
| | 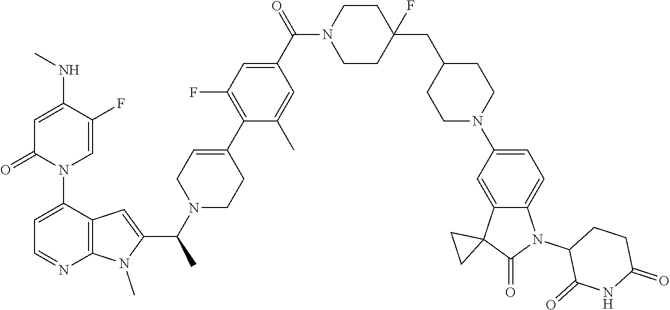 [SMILES: CNc1cc(=O)n(-c2ccnc3c2cc([C@H](C)N2CC=C(c4c(C)cc(C(=O)N5CCC(F)(CC6CCN(c7ccc8c(c7)C7(CC7)C(=O)N8C7CCC(=O)NC7=O)CC6)CC5)cc4F)CC2)n3C)cc1F] |
| 3u200 | 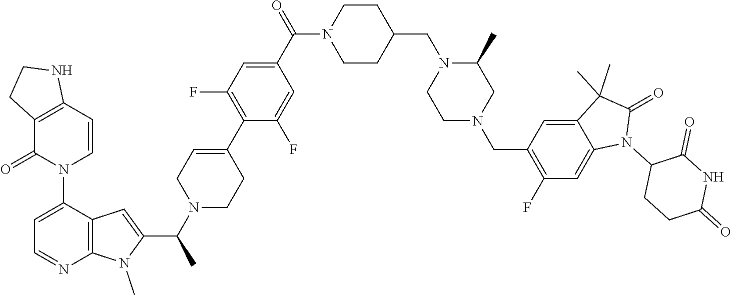 [SMILES: C[C@H]1CN(Cc2cc3c(cc2F)N(C2CCC(=O)NC2=O)C(=O)C3(C)C)CCN1CC1CCN(C(=O)c2cc(F)c(C3=CCN([C@@H](C)c4cc5c(-n6ccc7c(c6=O)CCN7)ccnc5n4C)CC3)c(F)c2)CC1] |
| 3u201 | 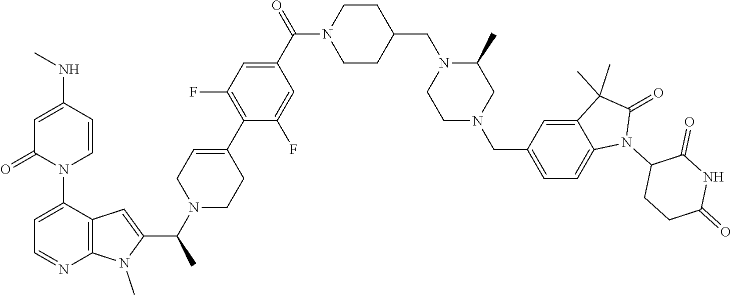 [SMILES: CNc1ccn(-c2ccnc3c2cc([C@H](C)N2CC=C(c4c(F)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)c(=O)c1] |
| 3u202 and 3u203 | 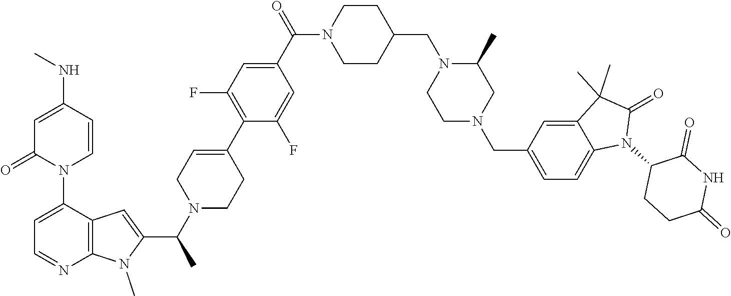 [SMILES: CNc1ccn(-c2ccnc3c2cc([C@H](C)N2CC=C(c4c(F)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)c(=O)c1] |
| | 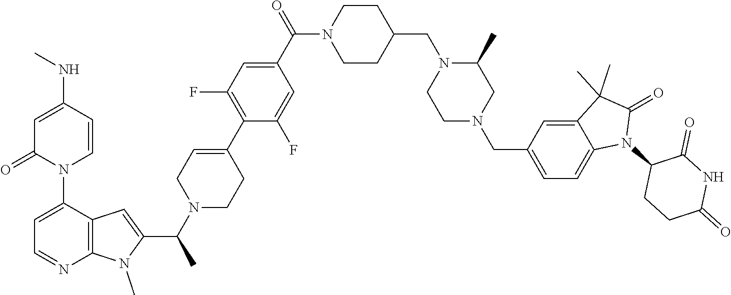 [SMILES: CNc1ccn(-c2ccnc3c2cc([C@H](C)N2CC=C(c4c(F)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)c(=O)c1] |
| 3u204 | 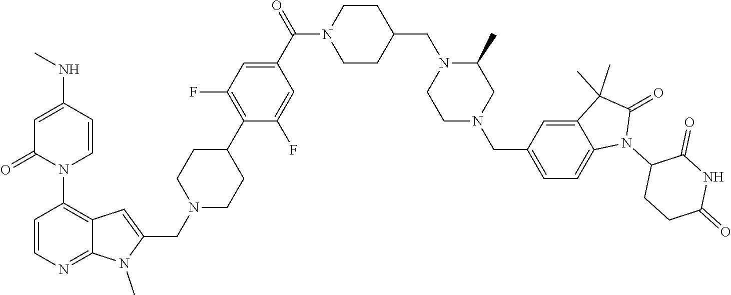 [SMILES: CNc1ccn(-c2ccnc3c2cc(CN2CCC(c4c(F)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)c(=O)c1] |
| 3u205 and 3u206 | 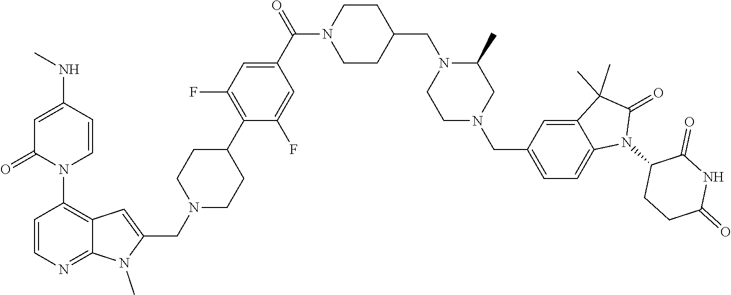 [SMILES: CNc1ccn(-c2ccnc3c2cc(CN2CCC(c4c(F)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)c(=O)c1] |
| | 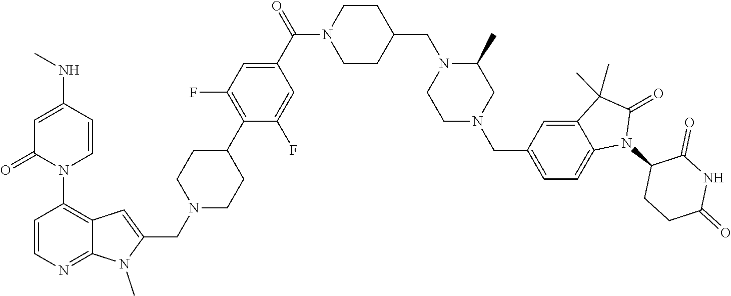 [SMILES: CNc1ccn(-c2ccnc3c2cc(CN2CCC(c4c(F)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)c(=O)c1] |
| 3u207 | 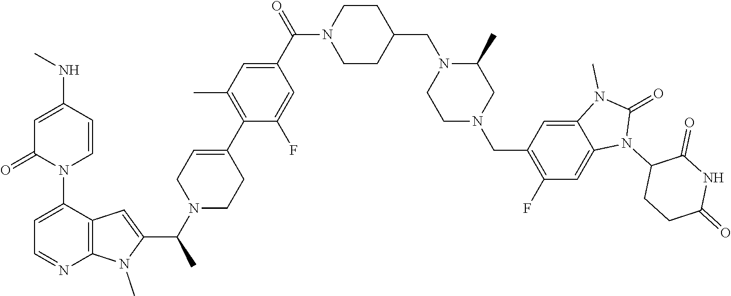 [SMILES: CNc1ccn(-c2ccnc3c2cc([C@H](C)N2CC=C(c4c(C)cc(C(=O)N5CCC(CN6CCN(Cc7cc8c(cc7F)n(C7CCC(=O)NC7=O)c(=O)n8C)C[C@@H]6C)CC5)cc4F)CC2)n3C)c(=O)c1] |
| 3u208 and 3u209 | 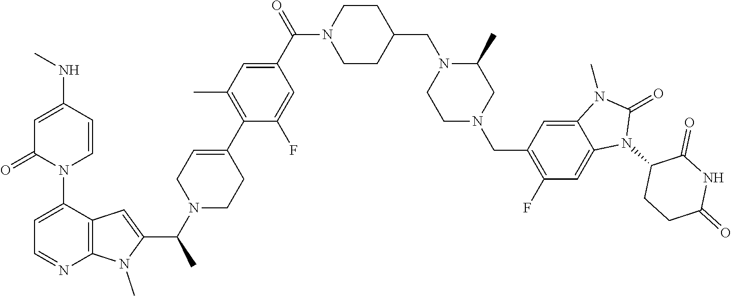 [SMILES: CNc1ccn(-c2ccnc3c2cc([C@H](C)N2CC=C(c4c(C)cc(C(=O)N5CCC(CN6CCN(Cc7cc8c(cc7F)n([C@H]7CCC(=O)NC7=O)c(=O)n8C)C[C@@H]6C)CC5)cc4F)CC2)n3C)c(=O)c1] |
| | 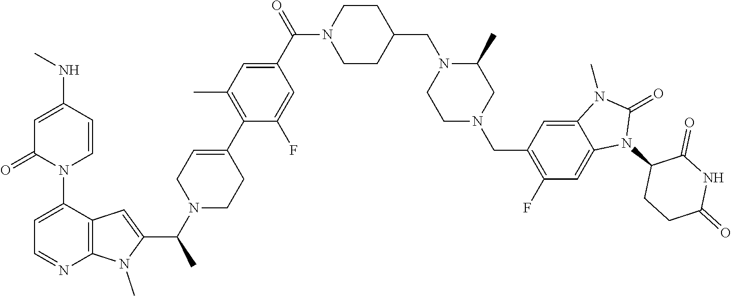 [SMILES: CNc1ccn(-c2ccnc3c2cc([C@H](C)N2CC=C(c4c(C)cc(C(=O)N5CCC(CN6CCN(Cc7cc8c(cc7F)n([C@@H]7CCC(=O)NC7=O)c(=O)n8C)C[C@@H]6C)CC5)cc4F)CC2)n3C)c(=O)c1] |
| 3u210 | 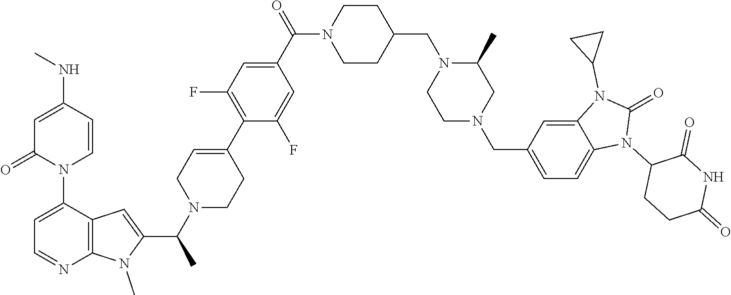 [SMILES: CNc1ccn(-c2ccnc3c2cc([C@H](C)N2CC=C(c4c(F)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)c(=O)c1] |
| 3u211 and 3u212 | 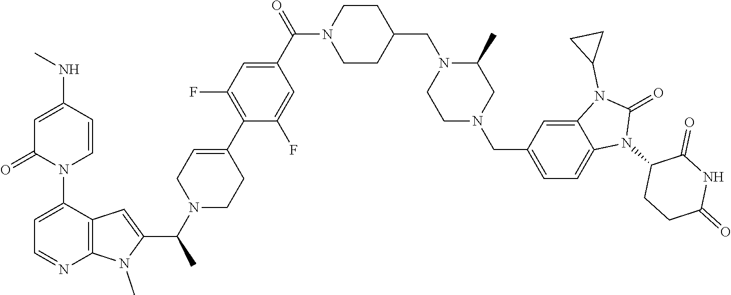 [SMILES: CNc1ccn(-c2ccnc3c2cc([C@H](C)N2CC=C(c4c(F)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)c(=O)c1] |
| | 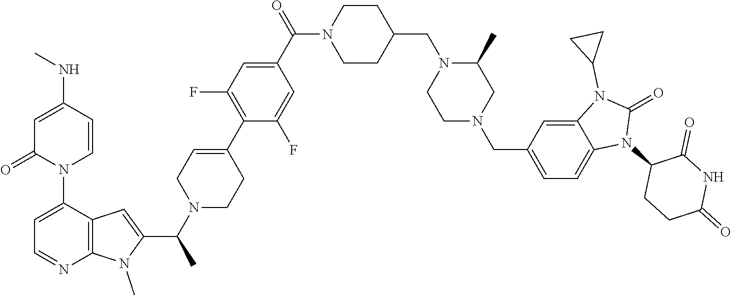 [SMILES: CNc1ccn(-c2ccnc3c2cc([C@H](C)N2CC=C(c4c(F)cc(C(=O)N5CCC(CN6CCN(Cc7ccc8c(c7)n(C7CC7)c(=O)n8[C@@H]7CCC(=O)NC7=O)C[C@@H]6C)CC5)cc4F)CC2)n3C)c(=O)c1] |
| 3u213 | 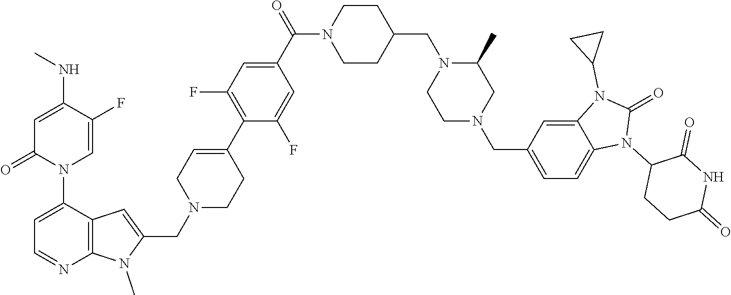 [SMILES: CNc1cc(=O)n(-c2ccnc3c2cc(CN2CC=C(c4c(F)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)cc1F] |
| 3u214 and 3u215 | 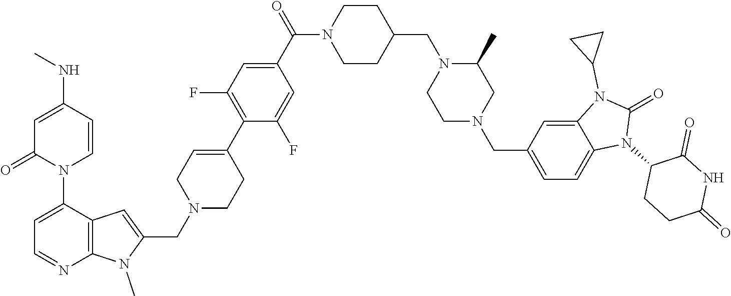 [SMILES: CNc1ccn(-c2ccnc3c2cc(CN2CC=C(c4c(F)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)c(=O)c1] |
| | 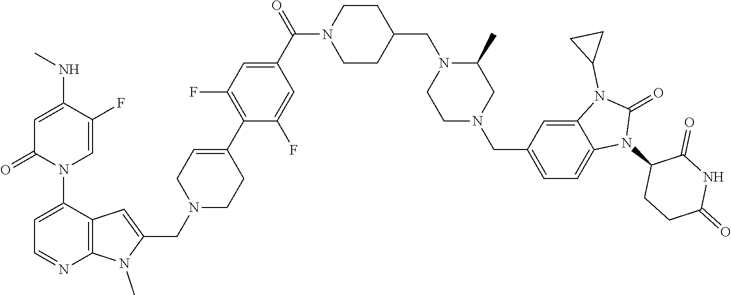 [SMILES: CNc1cc(=O)n(-c2ccnc3c2cc(CN2CC=C(c4c(F)cc(C(=O)N5CCC(CN6CCN(Cc7ccc8c(c7)n(C7CC7)c(=O)n8[C@@H]7CCC(=O)NC7=O)C[C@@H]6C)CC5)cc4F)CC2)n3C)cc1F] |
| 3u216 | 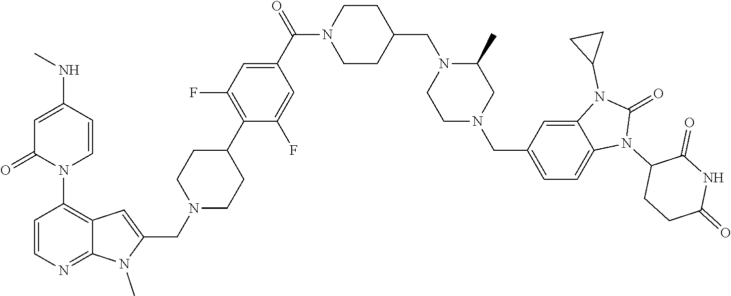 [SMILES: CNc1ccn(-c2ccnc3c2cc(CN2CCC(c4c(F)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)c(=O)c1] |
| 3u217 and 3u218 | 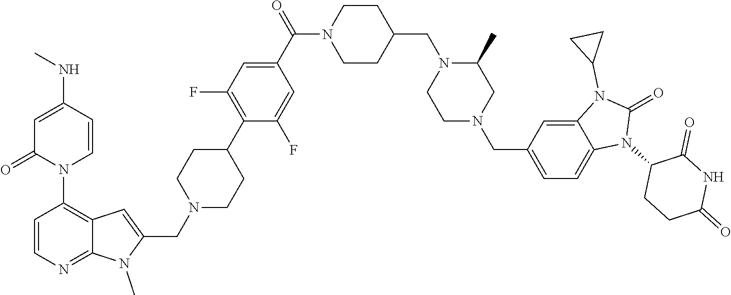 [SMILES: CNc1ccn(-c2ccnc3c2cc(CN2CCC(c4c(F)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)c(=O)c1] |
| | 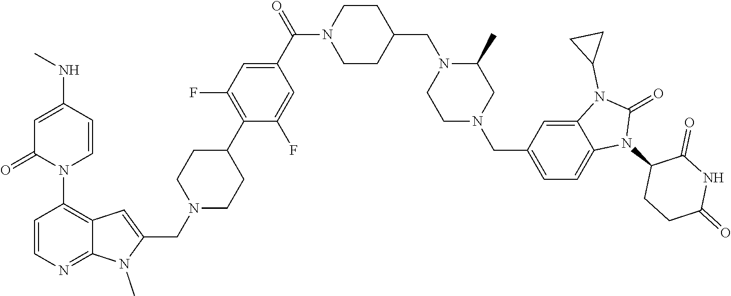 [SMILES: CNc1ccn(-c2ccnc3c2cc(CN2CCC(c4c(F)cc(C(=O)N5CCC(CN6CCN(Cc7ccc8c(c7)n(C7CC7)c(=O)n8[C@@H]7CCC(=O)NC7=O)C[C@@H]6C)CC5)cc4F)CC2)n3C)c(=O)c1] |
| 3u219 | 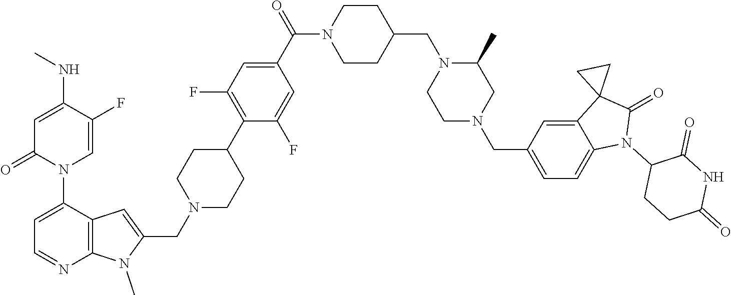 [SMILES: CNc1cc(=O)n(-c2ccnc3c2cc(CN2CCC(c4c(F)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)cc1F] |
| 3u22 | 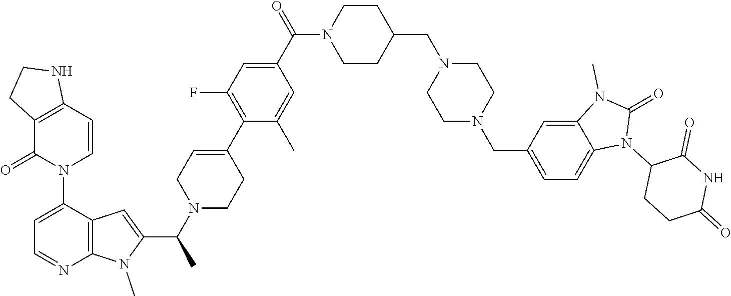 [SMILES: Cc1cc(C(=O)N2CCC(CN3CCN(Cc4ccc5c(c4)n(C)c(=O)n5C4CCC(=O)NC4=O)CC3)CC2)cc(F)c1C1=CCN([C@@H](C)c2cc3c(-n4ccc5c(c4=O)CCN5)ccnc3n2C)CC1] |
| 3u220 and 3u221 | 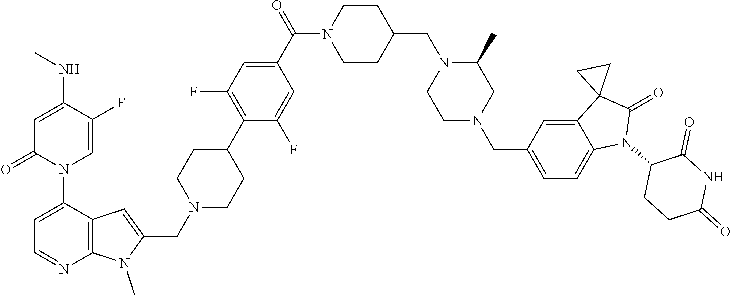 [SMILES: CNc1cc(=O)n(-c2ccnc3c2cc(CN2CCC(c4c(F)cc(C(=O)N5CCC(CN6CCN(Cc7ccc8c(c7)C7(CC7)C(=O)N8[C@H]7CCC(=O)NC7=O)C[C@@H]6C)CC5)cc4F)CC2)n3C)cc1F] |
| | 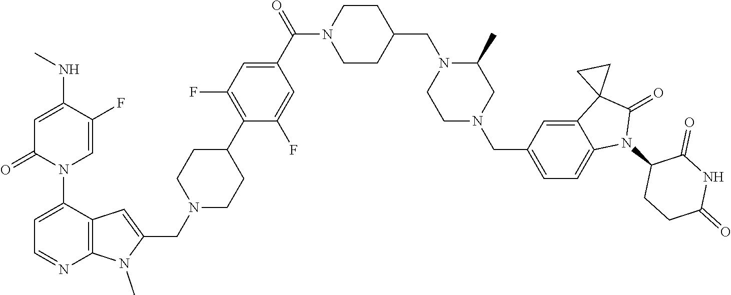 [SMILES: CNc1cc(=O)n(-c2ccnc3c2cc(CN2CCC(c4c(F)cc(C(=O)N5CCC(CN6CCN(Cc7ccc8c(c7)C7(CC7)C(=O)N8[C@@H]7CCC(=O)NC7=O)C[C@@H]6C)CC5)cc4F)CC2)n3C)cc1F] |
| 3u222 | 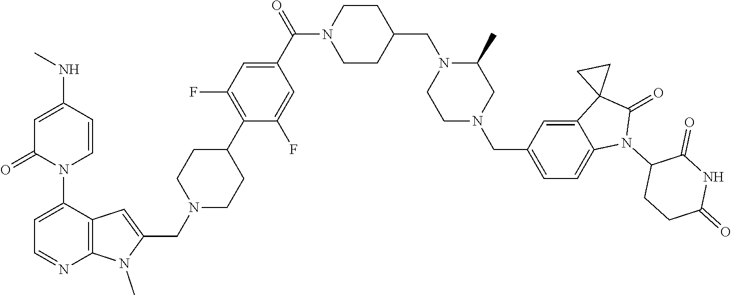 [SMILES: CNc1ccn(-c2ccnc3c2cc(CN2CCC(c4c(F)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)c(=O)c1] |
| 3u223 and 3u224 | 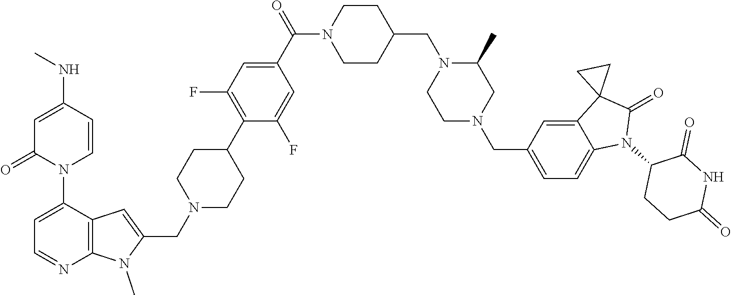 [SMILES: CNc1ccn(-c2ccnc3c2cc(CN2CCC(c4c(F)cc(C(=O)N5CCC(CN6CCN(Cc7ccc8c(c7)C7(CC7)C(=O)N8[C@H]7CCC(=O)NC7=O)C[C@@H]6C)CC5)cc4F)CC2)n3C)c(=O)c1] |
| | 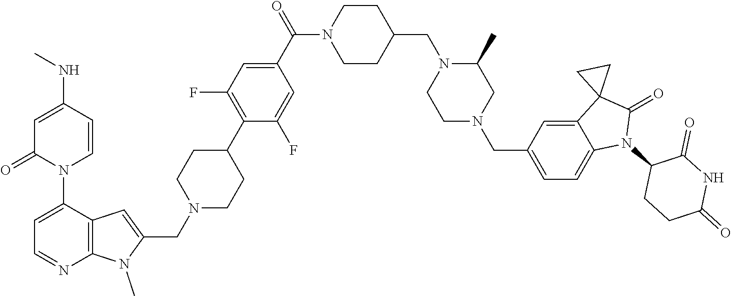 [SMILES: CNc1ccn(-c2ccnc3c2cc(CN2CCC(c4c(F)cc(C(=O)N5CCC(CN6CCN(Cc7ccc8c(c7)C7(CC7)C(=O)N8[C@@H]7CCC(=O)NC7=O)C[C@@H]6C)CC5)cc4F)CC2)n3C)c(=O)c1] |
| 3u225 | 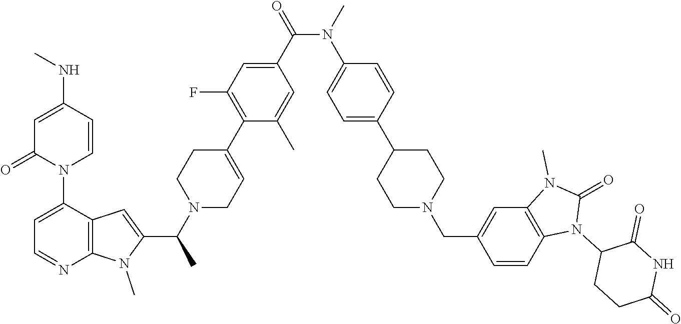 [SMILES: CNc1ccn(-c2ccnc3c2cc([C@H](C)N2CC=C(c4c(C)cc(C(=O)N(C)c5ccc(C6CCN(Cc7ccc8c(c7)n(C)c(=O)n8C7CCC(=O)NC7=O)CC6)cc5)cc4F)CC2)n3C)c(=O)c1] |
| 3u226 and 3u227 | 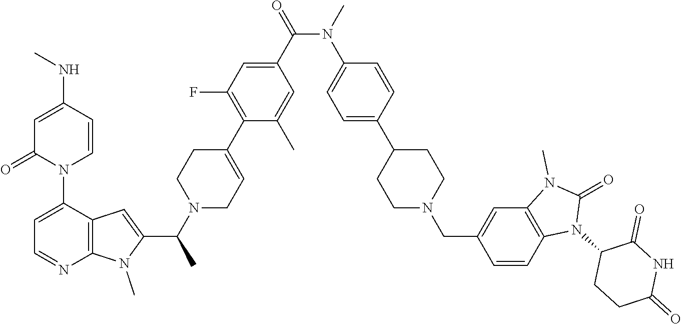 [SMILES: CNc1ccn(-c2ccnc3c2cc([C@H](C)N2CC=C(c4c(C)cc(C(=O)N(C)c5ccc(C6CCN(Cc7ccc8c(c7)n(C)c(=O)n8[C@H]7CCC(=O)NC7=O)CC6)cc5)cc4F)CC2)n3C)c(=O)c1] |
| | 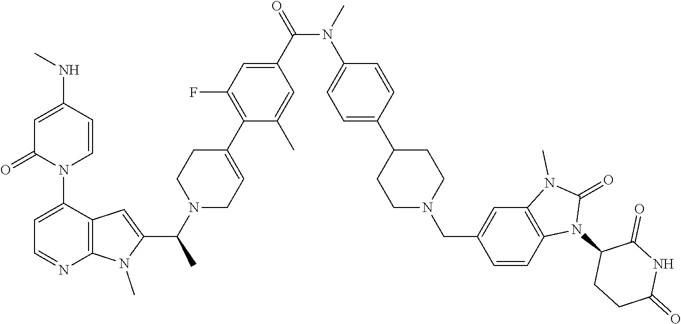 [SMILES: CNc1ccn(-c2ccnc3c2cc([C@H](C)N2CC=C(c4c(C)cc(C(=O)N(C)c5ccc(C6CCN(Cc7ccc8c(c7)n(C)c(=O)n8[C@@H]7CCC(=O)NC7=O)CC6)cc5)cc4F)CC2)n3C)c(=O)c1] |
| 3u228 | 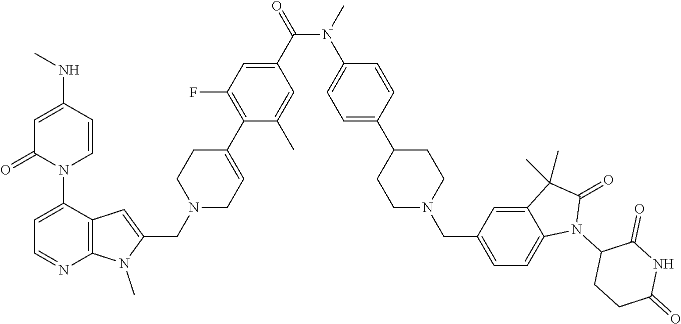 [SMILES: CNc1ccn(-c2ccnc3c2cc(CN2CC=C(c4c(C)cc(C(=O)N(C)c5ccc(C6CCN(Cc7ccc8c(c7)C(C)(C)C(=O)N8C7CCC(=O)NC7=O)CC6)cc5)cc4F)CC2)n3C)c(=O)c1] |
| 3u229 and 3u230 | 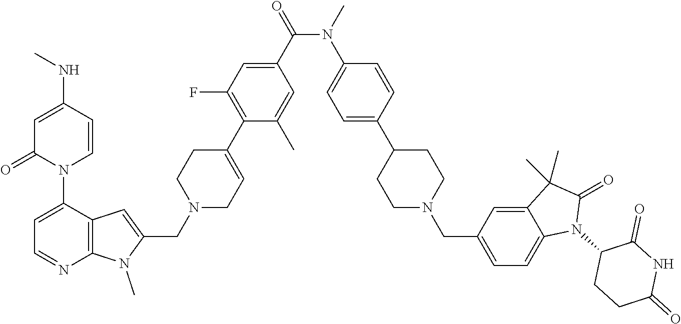 [SMILES: CNc1ccn(-c2ccnc3c2cc(CN2CC=C(c4c(C)cc(C(=O)N(C)c5ccc(C6CCN(Cc7ccc8c(c7)C(C)(C)C(=O)N8[C@H]7CCC(=O)NC7=O)CC6)cc5)cc4F)CC2)n3C)c(=O)c1] |
| | 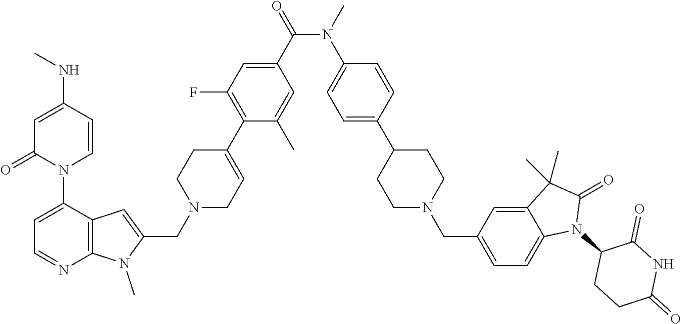 [SMILES: CNc1ccn(-c2ccnc3c2cc(CN2CC=C(c4c(C)cc(C(=O)N(C)c5ccc(C6CCN(Cc7ccc8c(c7)C(C)(C)C(=O)N8[C@@H]7CCC(=O)NC7=O)CC6)cc5)cc4F)CC2)n3C)c(=O)c1] |
| 3u23 | 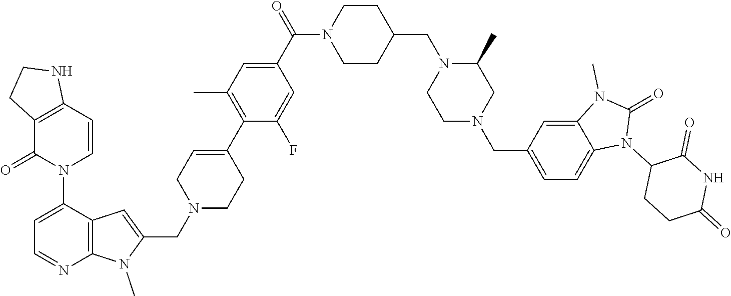 [SMILES: Cc1cc(C(=O)N2CCC(CN3CCN(Cc4ccc5c(c4)n(C)c(=O)n5C4CCC(=O)NC4=O)C[C@@H]3C)CC2)cc(F)c1C1=CCN(Cc2cc3c(-n4ccc5c(c4=O)CCN5)ccnc3n2C)CC1] |
| 3u231 | 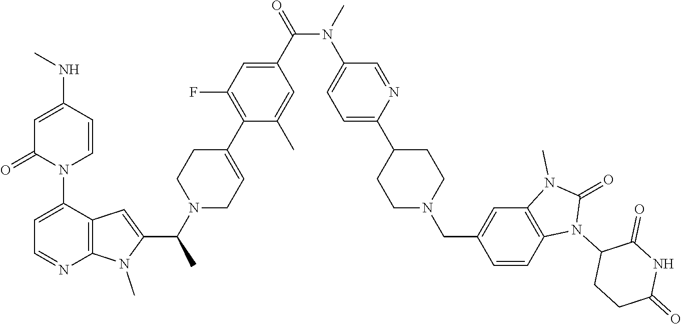 [SMILES: CNc1ccn(-c2ccnc3c2cc([C@H](C)N2CC=C(c4c(C)cc(C(=O)N(C)c5ccc(C6CCN(Cc7ccc8c(c7)n(C)c(=O)n8C7CCC(=O)NC7=O)CC6)nc5)cc4F)CC2)n3C)c(=O)c1] |
| 3u232 | 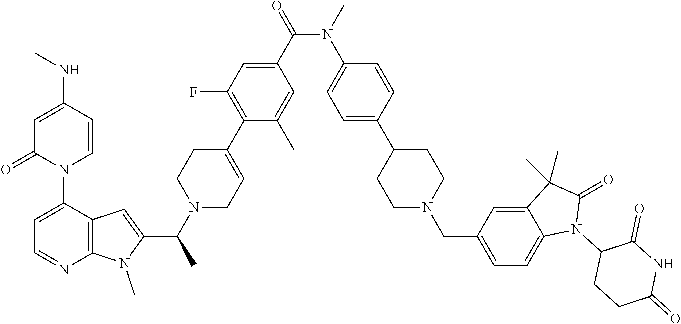 [SMILES: CNc1ccn(-c2ccnc3c2cc([C@H](C)N2CC=C(c4c(C)cc(C(=O)N(C)c5ccc(C6CCN(Cc7ccc8c(c7)C(C)(C)C(=O)N8C7CCC(=O)NC7=O)CC6)cc5)cc4F)CC2)n3C)c(=O)c1] |
| 3u233 | 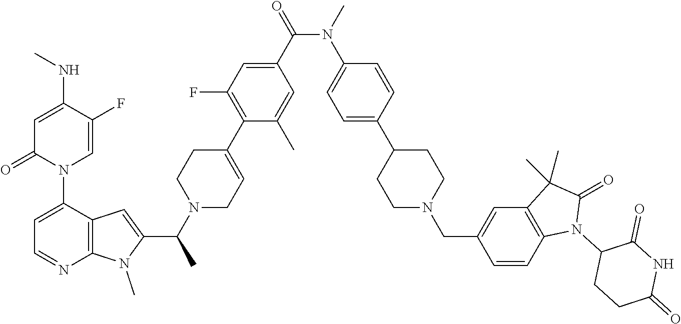 [SMILES: CNc1cc(=O)n(-c2ccnc3c2cc([C@H](C)N2CC=C(c4c(C)cc(C(=O)N(C)c5ccc(C6CCN(Cc7ccc8c(c7)C(C)(C)C(=O)N8C7CCC(=O)NC7=O)CC6)cc5)cc4F)CC2)n3C)cc1F] |
| 3u234 and 3u235 | 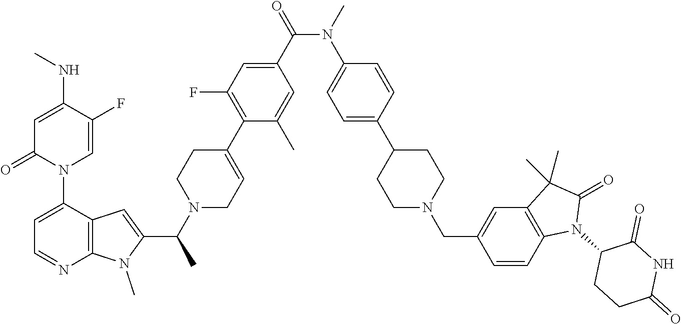 [SMILES: CNc1cc(=O)n(-c2ccnc3c2cc([C@H](C)N2CC=C(c4c(C)cc(C(=O)N(C)c5ccc(C6CCN(Cc7ccc8c(c7)C(C)(C)C(=O)N8[C@H]7CCC(=O)NC7=O)CC6)cc5)cc4F)CC2)n3C)cc1F] |
| | 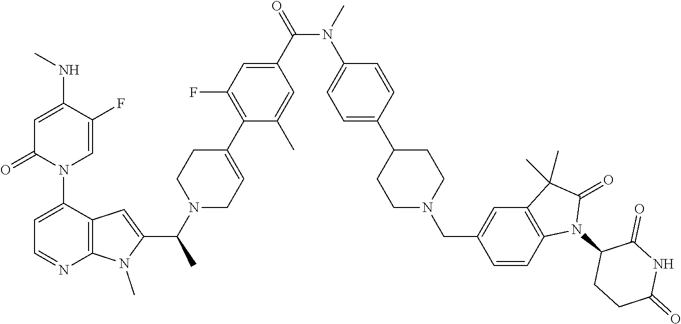 [SMILES: CNc1cc(=O)n(-c2ccnc3c2cc([C@H](C)N2CC=C(c4c(C)cc(C(=O)N(C)c5ccc(C6CCN(Cc7ccc8c(c7)C(C)(C)C(=O)N8[C@@H]7CCC(=O)NC7=O)CC6)cc5)cc4F)CC2)n3C)cc1F] |
| 3u236 | 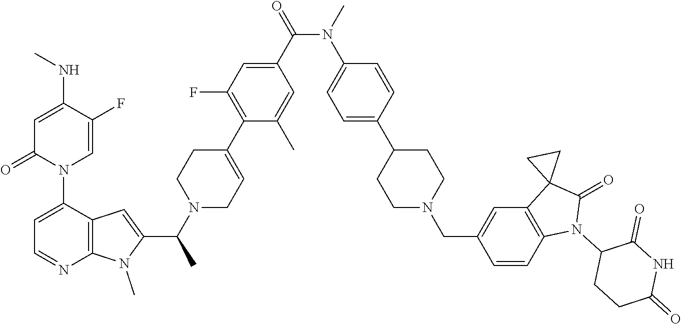 [SMILES: CNc1cc(=O)n(-c2ccnc3c2cc([C@H](C)N2CC=C(c4c(C)cc(C(=O)N(C)c5ccc(C6CCN(Cc7ccc8c(c7)C7(CC7)C(=O)N8C7CCC(=O)NC7=O)CC6)cc5)cc4F)CC2)n3C)cc1F] |
| 3u237 | 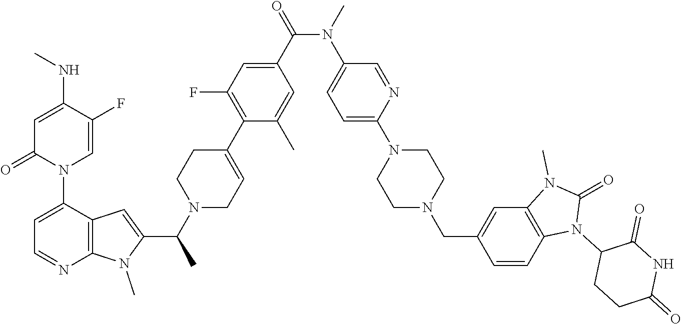 [SMILES: CNc1cc(=O)n(-c2ccnc3c2cc([C@H](C)N2CC=C(c4c(C)cc(C(=O)N(C)c5ccc(N6CCN(Cc7ccc8c(c7)n(C)c(=O)n8C7CCC(=O)NC7=O)CC6)nc5)cc4F)CC2)n3C)cc1F] |
| 3u238 | 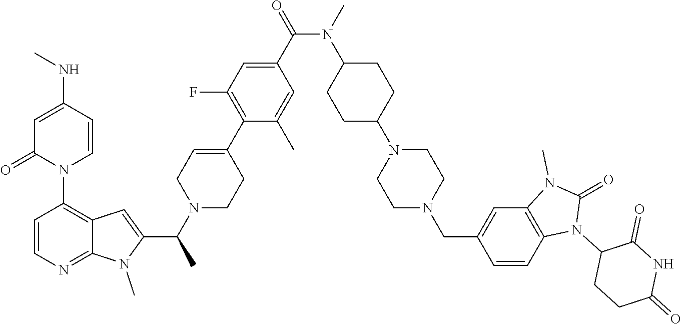 [SMILES: CNc1ccn(-c2ccnc3c2cc([C@H](C)N2CC=C(c4c(C)cc(C(=O)N(C)C5CCC(N6CCN(Cc7ccc8c(c7)n(C)c(=O)n8C7CCC(=O)NC7=O)CC6)CC5)cc4F)CC2)n3C)c(=O)c1] |
| 3u239 | 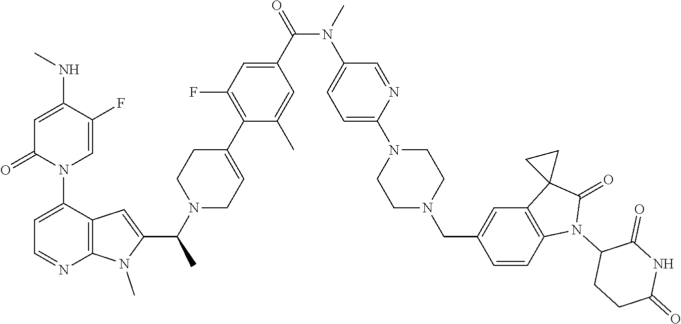 [SMILES: CNc1cc(=O)n(-c2ccnc3c2cc([C@H](C)N2CC=C(c4c(C)cc(C(=O)N(C)c5ccc(N6CCN(Cc7ccc8c(c7)C7(CC7)C(=O)N8C7CCC(=O)NC7=O)CC6)nc5)cc4F)CC2)n3C)cc1F] |
| 3u24 and 3u25 | 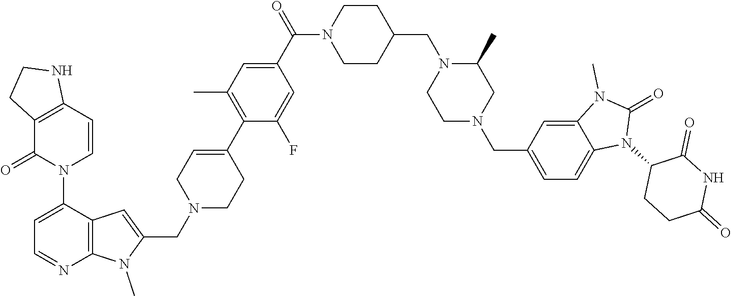 [SMILES: Cc1cc(C(=O)N2CCC(CN3CCN(Cc4ccc5c(c4)n(C)c(=O)n5[C@H]4CCC(=O)NC4=O)C[C@@H]3C)CC2)cc(F)c1C1=CCN(Cc2cc3c(-n4ccc5c(c4=O)CCN5)ccnc3n2C)CC1] |
| | 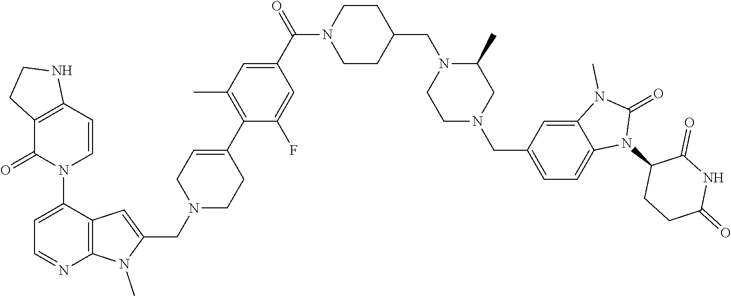 [SMILES: Cc1cc(C(=O)N2CCC(CN3CCN(Cc4ccc5c(c4)n(C)c(=O)n5[C@@H]4CCC(=O)NC4=O)C[C@@H]3C)CC2)cc(F)c1C1=CCN(Cc2cc3c(-n4ccc5c(c4=O)CCN5)ccnc3n2C)CC1] |
| 3u240 | 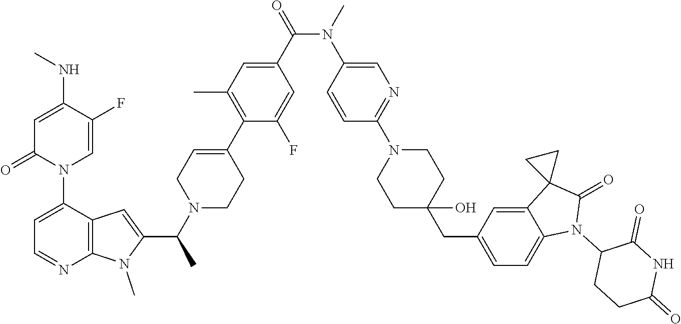 [SMILES: CNc1cc(=O)n(-c2ccnc3c2cc([C@H](C)N2CC=C(c4c(C)cc(C(=O)N(C)c5ccc(N6CCC(O)(Cc7ccc8c(c7)C7(CC7)C(=O)N8C7CCC(=O)NC7=O)CC6)nc5)cc4F)CC2)n3C)cc1F] |
| 3u241 | 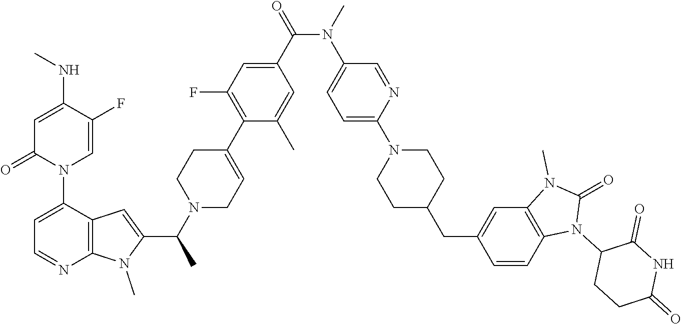 [SMILES: CNc1cc(=O)n(-c2ccnc3c2cc([C@H](C)N2CC=C(c4c(C)cc(C(=O)N(C)c5ccc(N6CCC(Cc7ccc8c(c7)n(C)c(=O)n8C7CCC(=O)NC7=O)CC6)nc5)cc4F)CC2)n3C)cc1F] |
| 3u242 and 3u243 | 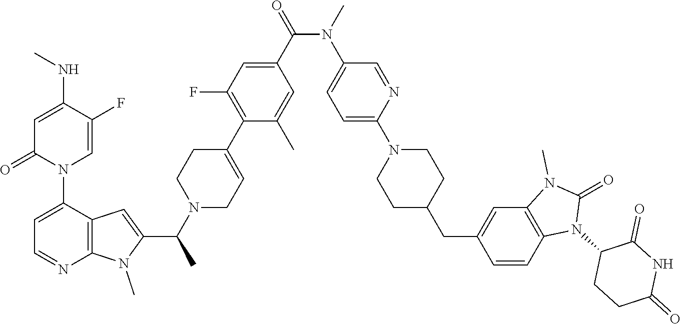 [SMILES: CNc1cc(=O)n(-c2ccnc3c2cc([C@H](C)N2CC=C(c4c(C)cc(C(=O)N(C)c5ccc(N6CCC(Cc7ccc8c(c7)n(C)c(=O)n8[C@H]7CCC(=O)NC7=O)CC6)nc5)cc4F)CC2)n3C)cc1F] |
| | 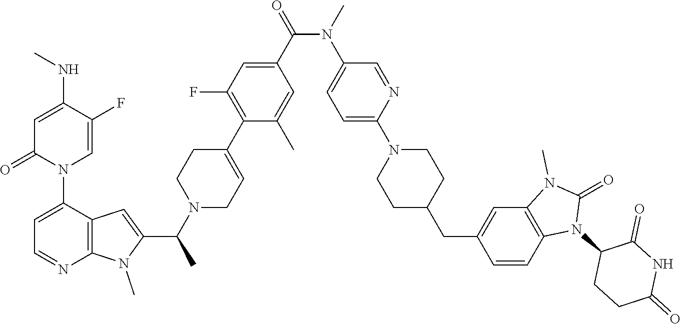 [SMILES: CNc1cc(=O)n(-c2ccnc3c2cc([C@H](C)N2CC=C(c4c(C)cc(C(=O)N(C)c5ccc(N6CCC(Cc7ccc8c(c7)n(C)c(=O)n8[C@@H]7CCC(=O)NC7=O)CC6)nc5)cc4F)CC2)n3C)cc1F] |
| 3u244 | 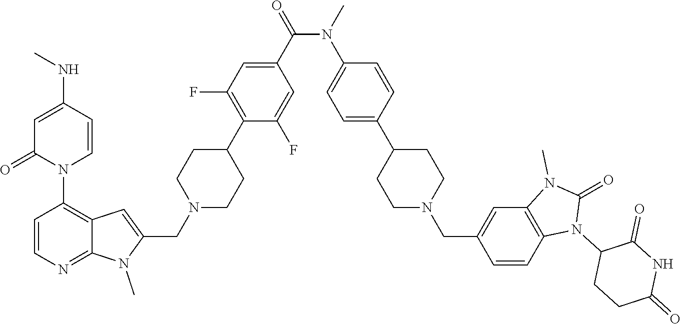 [SMILES: CNc1ccn(-c2ccnc3c2cc(CN2CCC(c4c(F)cc(C(=O)N(C)c5ccc(C6CCN(Cc7ccc8c(c7)n(C)c(=O)n8C7CCC(=O)NC7=O)CC6)cc5)cc4F)CC2)n3C)c(=O)c1] |
| 3u245 | 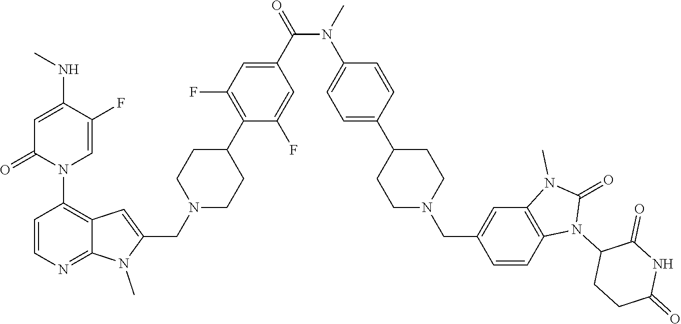 [SMILES: CNc1cc(=O)n(-c2ccnc3c2cc(CN2CCC(c4c(F)cc(C(=O)N(C)c5ccc(C6CCN(Cc7ccc8c(c7)n(C)c(=O)n8C7CCC(=O)NC7=O)CC6)cc5)cc4F)CC2)n3C)cc1F] |
| 3u246 | 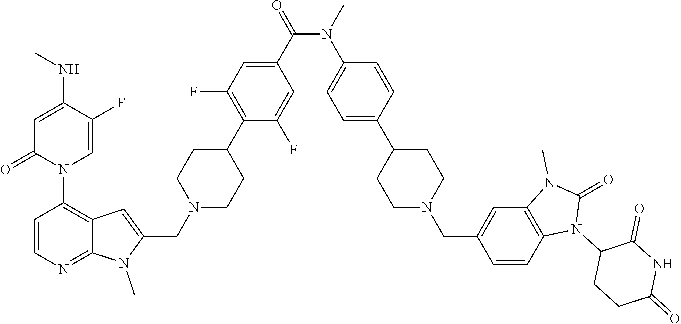 [SMILES: CNc1cc(=O)n(-c2ccnc3c2cc(CN2CCC(c4c(F)cc(C(=O)N(C)c5ccc(C6CCN(Cc7ccc8c(c7)n(C)c(=O)n8C7CCC(=O)NC7=O)CC6)cc5)cc4F)CC2)n3C)cc1F] |
| 3u247 | 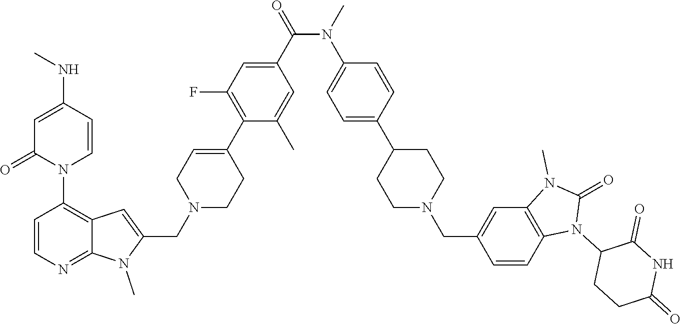 [SMILES: CNc1ccn(-c2ccnc3c2cc(CN2CC=C(c4c(C)cc(C(=O)N(C)c5ccc(C6CCN(Cc7ccc8c(c7)n(C)c(=O)n8C7CCC(=O)NC7=O)CC6)cc5)cc4F)CC2)n3C)c(=O)c1] |
| 3u248 | 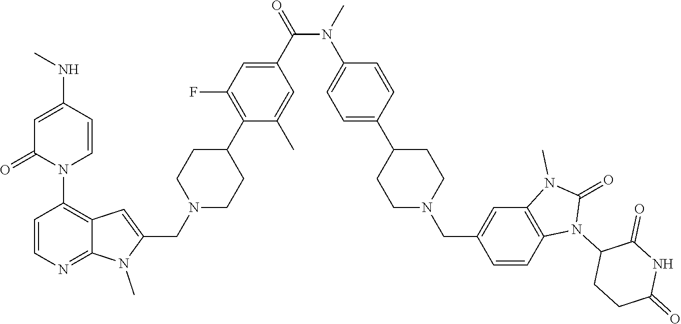 [SMILES: CNc1ccn(-c2ccnc3c2cc(CN2CCC(c4c(C)cc(C(=O)N(C)c5ccc(C6CCN(Cc7ccc8c(c7)n(C)c(=O)n8C7CCC(=O)NC7=O)CC6)cc5)cc4F)CC2)n3C)c(=O)c1] |
| 3u249 | 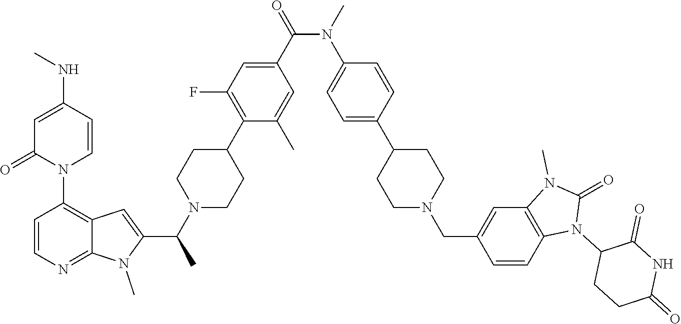 [SMILES: CNc1ccn(-c2ccnc3c2cc([C@H](C)N2CCC(c4c(C)cc(C(=O)N(C)c5ccc(C6CCN(Cc7ccc8c(c7)n(C)c(=O)n8C7CCC(=O)NC7=O)CC6)cc5)cc4F)CC2)n3C)c(=O)c1] |
| 3u250 | 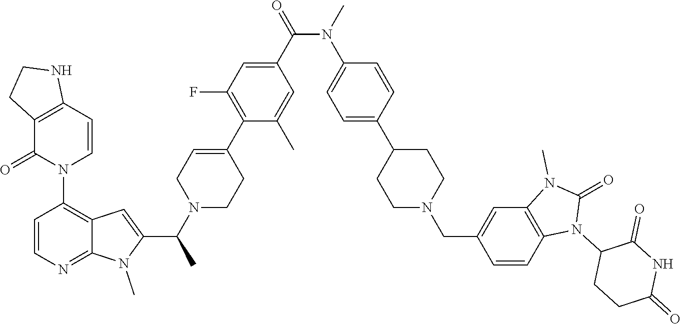 [SMILES: Cc1cc(C(=O)N(C)c2ccc(C3CCN(Cc4ccc5c(c4)n(C)c(=O)n5C4CCC(=O)NC4=O)CC3)cc2)cc(F)c1C1=CCN([C@@H](C)c2cc3c(-n4ccc5c(c4=O)CCN5)ccnc3n2C)CC1] |
| 3u251 | 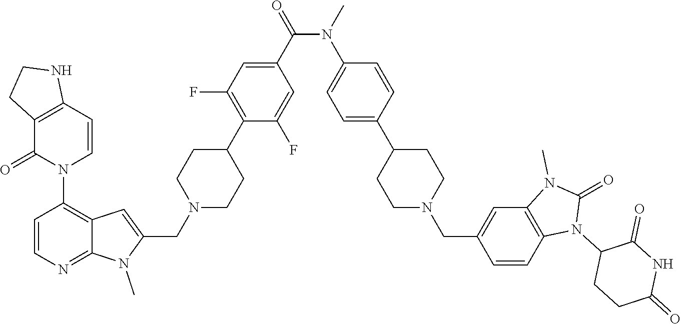 [SMILES: CN(C(=O)c1cc(F)c(C2CCN(Cc3cc4c(-n5ccc6c(c5=O)CCN6)ccnc4n3C)CC2)c(F)c1)c1ccc(C2CCN(Cc3ccc4c(c3)n(C)c(=O)n4C3CCC(=O)NC3=O)CC2)cc1] |
| 3u252 | 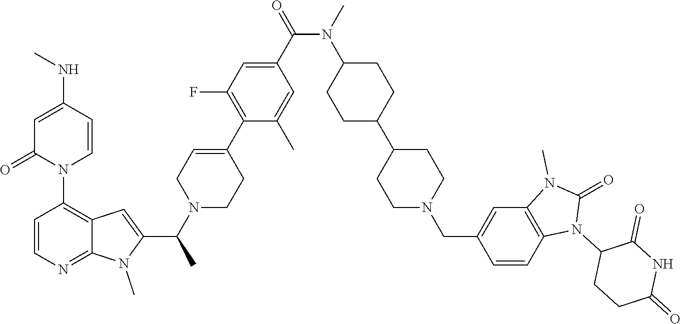 [SMILES: CNc1ccn(-c2ccnc3c2cc([C@H](C)N2CC=C(c4c(C)cc(C(=O)N(C)C5CCC(C6CCN(Cc7ccc8c(c7)n(C)c(=O)n8C7CCC(=O)NC7=O)CC6)CC5)cc4F)CC2)n3C)c(=O)c1] |
| 3u253 | 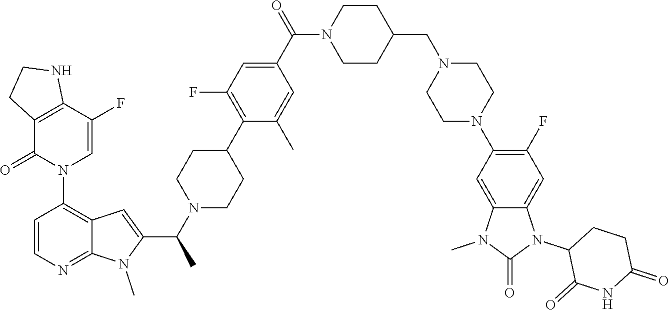 [SMILES: Cc1cc(C(=O)N2CCC(CN3CCN(c4cc5c(cc4F)n(C4CCC(=O)NC4=O)c(=O)n5C)CC3)CC2)cc(F)c1C1CCN([C@@H](C)c2cc3c(-n4cc(F)c5c(c4=O)CCN5)ccnc3n2C)CC1] |
| 3u254 | 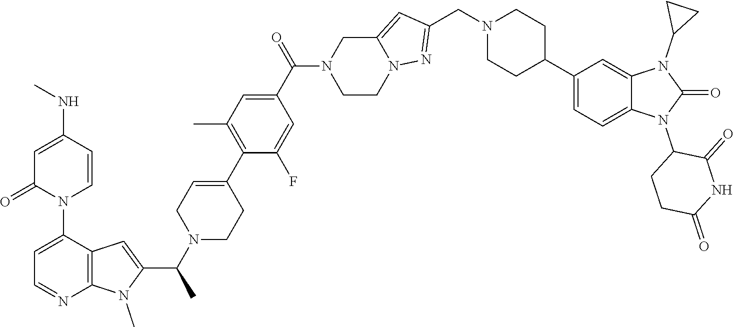 [SMILES: CNc1ccn(-c2ccnc3c2cc([C@H](C)N2CC=C(c4c(C)cc(C(=O)N5CCn6nc(CN7CCC(c8ccc9c(c8)n(C8CC8)c(=O)n9C8CCC(=O)NC8=O)CC7)cc6C5)cc4F)CC2)n3C)c(=O)c1] |
| 3u255 | 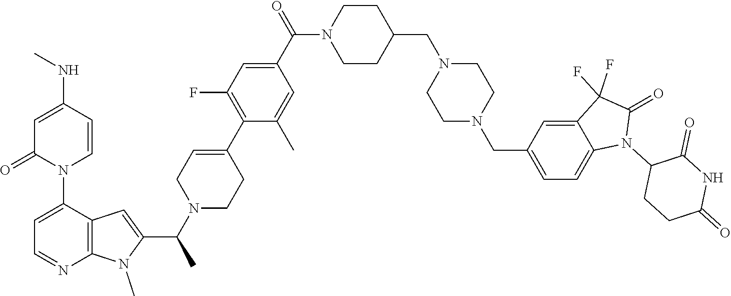 [SMILES: CNc1ccn(-c2ccnc3c2cc([C@H](C)N2CC=C(c4c(C)cc(C(=O)N5CCC(CN6CCN(Cc7ccc8c(c7)C(F)(F)C(=O)N8C7CCC(=O)NC7=O)CC6)CC5)cc4F)CC2)n3C)c(=O)c1] |
| 3u256 | 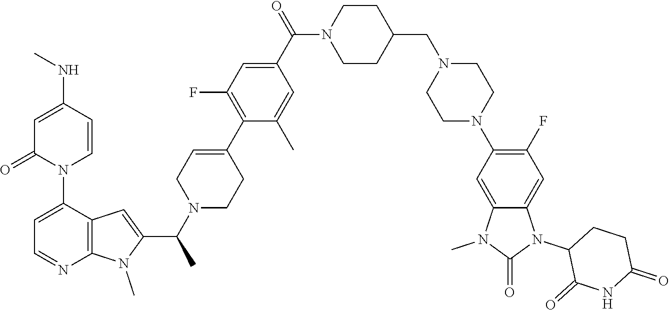 [SMILES: CNc1ccn(-c2ccnc3c2cc([C@H](C)N2CC=C(c4c(C)cc(C(=O)N5CCC(CN6CCN(c7cc8c(cc7F)n(C7CCC(=O)NC7=O)c(=O)n8C)CC6)CC5)cc4F)CC2)n3C)c(=O)c1] |
| 3u257 | 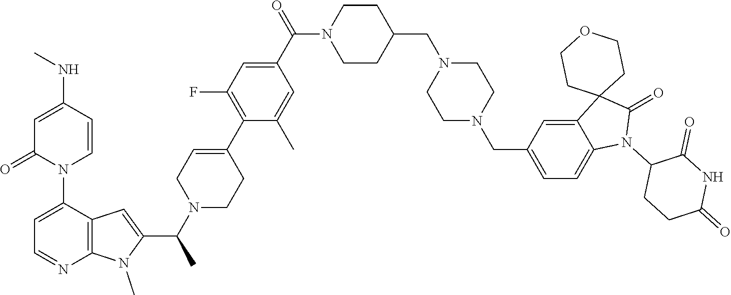 [SMILES: CNc1ccn(-c2ccnc3c2cc([C@H](C)N2CC=C(c4c(C)cc(C(=O)N5CCC(CN6CCN(Cc7ccc8c(c7)C7(CCOCC7)C(=O)N8C7CCC(=O)NC7=O)CC6)CC5)cc4F)CC2)n3C)c(=O)c1] |
| 3u258 | 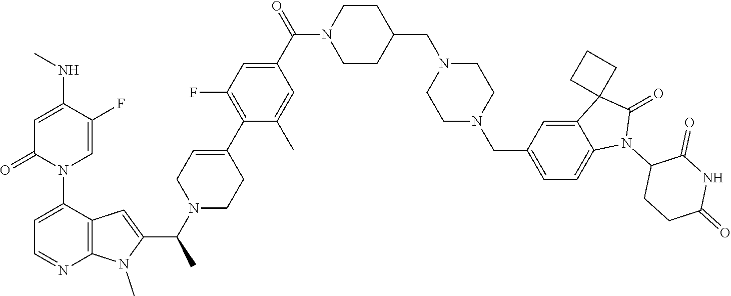 [SMILES: CNc1cc(=O)n(-c2ccnc3c2cc([C@H](C)N2CC=C(c4c(C)cc(C(=O)N5CCC(CN6CCN(Cc7ccc8c(c7)C7(CCC7)C(=O)N8C7CCC(=O)NC7=O)CC6)CC5)cc4F)CC2)n3C)cc1F] |
| 3u259 | 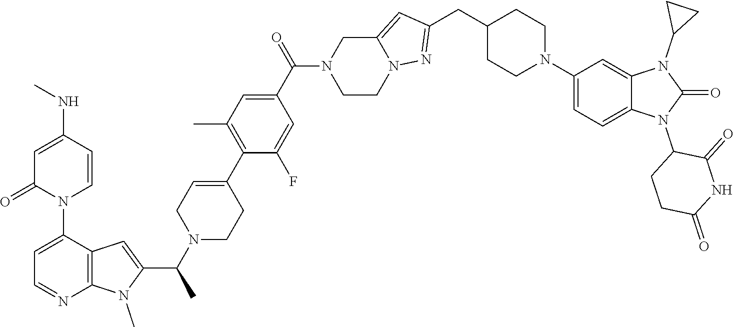 [SMILES: CNc1ccn(-c2ccnc3c2cc([C@H](C)N2CC=C(c4c(C)cc(C(=O)N5CCn6nc(CC7CCN(c8ccc9c(c8)n(C8CC8)c(=O)n9C8CCC(=O)NC8=O)CC7)cc6C5)cc4F)CC2)n3C)c(=O)c1] |
| 3u26 | 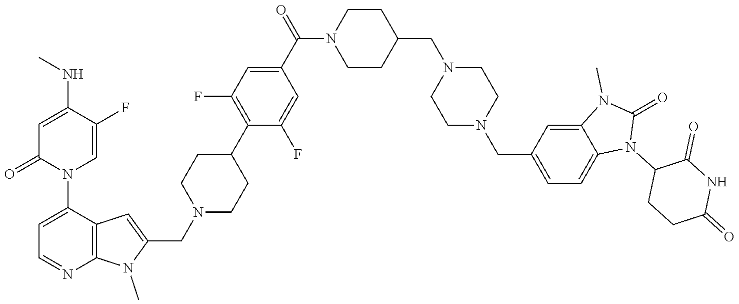 [SMILES: CNc1cc(=O)n(-c2ccnc3c2cc(CN2CCC(c4c(F)cc(C(=O)N5CCC(CN6CCN(Cc7ccc8c(c7)n(C)c(=O)n8C7CCC(=O)NC7=O)CC6)CC5)cc4F)CC2)n3C)cc1F] |
| 3u260 | 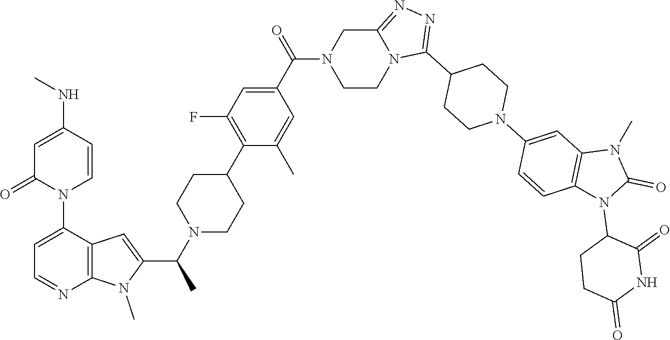 [SMILES: CNc1ccn(-c2ccnc3c2cc([C@H](C)N2CCC(c4c(C)cc(C(=O)N5CCn6c(nnc6C6CCN(c7ccc8c(c7)n(C)c(=O)n8C7CCC(=O)NC7=O)CC6)C5)cc4F)CC2)n3C)c(=O)c1] |
| 3u261 | 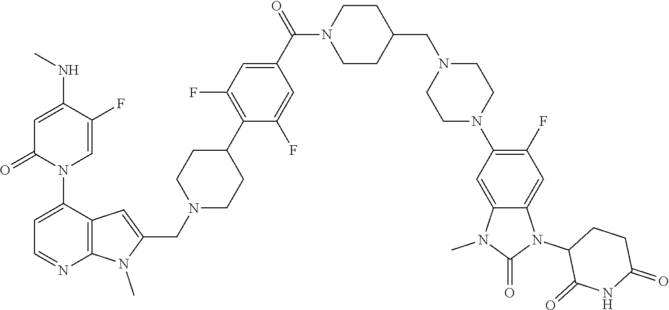 [SMILES: CNc1cc(=O)n(-c2ccnc3c2cc(CN2CCC(c4c(F)cc(C(=O)N5CCC(CN6CCN(c7cc8c(cc7F)n(C7CCC(=O)NC7=O)c(=O)n8C)CC6)CC5)cc4F)CC2)n3C)cc1F] |
| 3u262 | 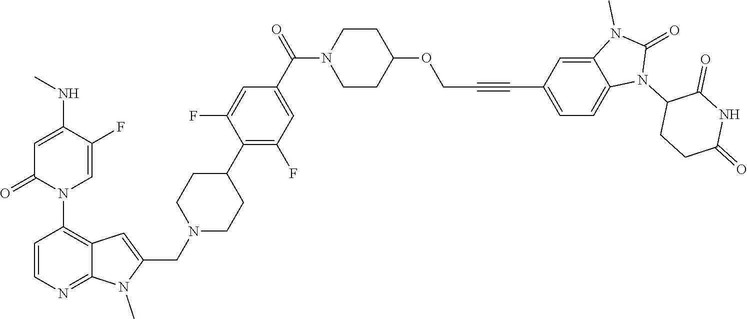 [SMILES: CNc1cc(=O)n(-c2ccnc3c2cc(CN2CCC(c4c(F)cc(C(=O)N5CCC(OCC#Cc6ccc7c(c6)n(C)c(=O)n7C6CCC(=O)NC6=O)CC5)cc4F)CC2)n3C)cc1F] |
| 3u263 | 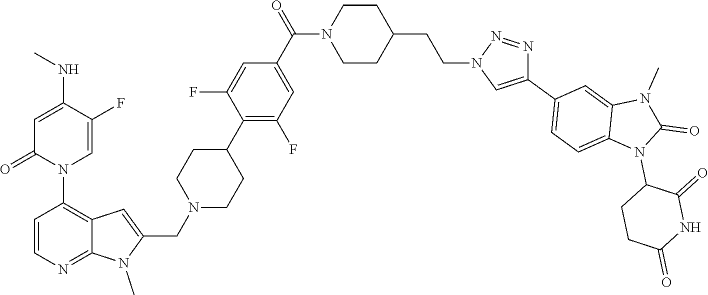 [SMILES: CNc1cc(=O)n(-c2ccnc3c2cc(CN2CCC(c4c(F)cc(C(=O)N5CCC(CCn6cc(-c7ccc8c(c7)n(C)c(=O)n8C7CCC(=O)NC7=O)nn6)CC5)cc4F)CC2)n3C)cc1F] |
| 3u264 | 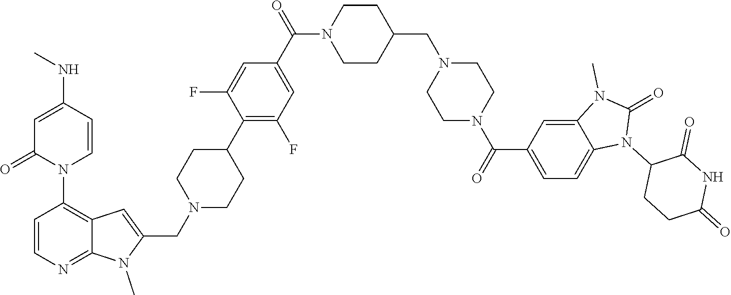 [SMILES: CNc1ccn(-c2ccnc3c2cc(CN2CCC(c4c(F)cc(C(=O)N5CCC(CN6CCN(C(=O)c7ccc8c(c7)n(C)c(=O)n8C7CCC(=O)NC7=O)CC6)CC5)cc4F)CC2)n3C)c(=O)c1] |
| 3u265 | 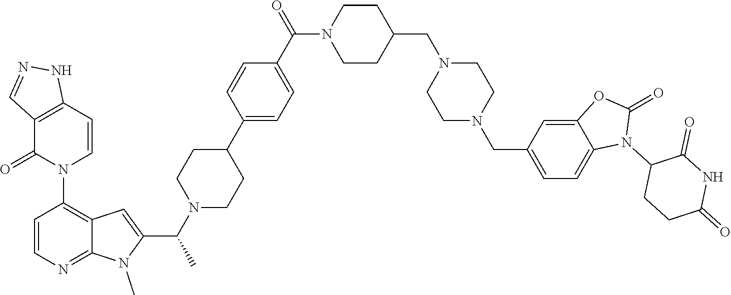 [SMILES: C[C@H](c1cc2c(-n3ccc4[nH]ncc4c3=O)ccnc2n1C)N1CCC(c2ccc(C(=O)N3CCC(CN4CCN(Cc5ccc6c(c5)oc(=O)n6C5CCC(=O)NC5=O)CC4)CC3)cc2)CC1] |
| 3u267 | 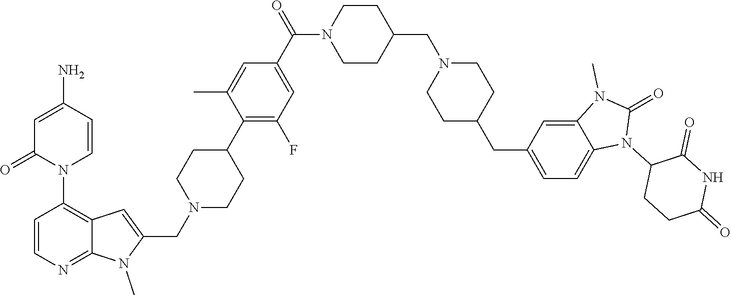 [SMILES: Cc1cc(C(=O)N2CCC(CN3CCC(Cc4ccc5c(c4)n(C)c(=O)n5C4CCC(=O)NC4=O)CC3)CC2)cc(F)c1C1CCN(Cc2cc3c(-n4ccc(N)cc4=O)ccnc3n2C)CC1] |
| 3u268 | 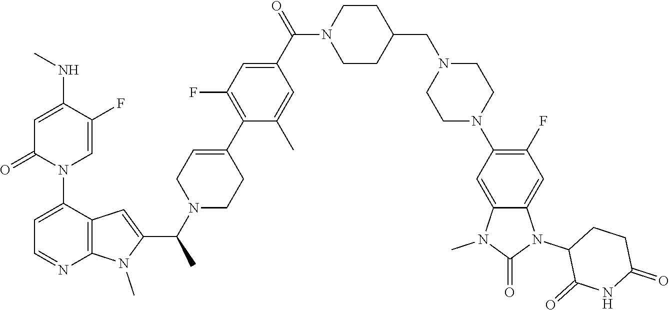 [SMILES: CNc1cc(=O)n(-c2ccnc3c2cc([C@H](C)N2CC=C(c4c(C)cc(C(=O)N5CCC(CN6CCN(c7cc8c(cc7F)n(C7CCC(=O)NC7=O)c(=O)n8C)CC6)CC5)cc4F)CC2)n3C)cc1F] |
| 3u269 | 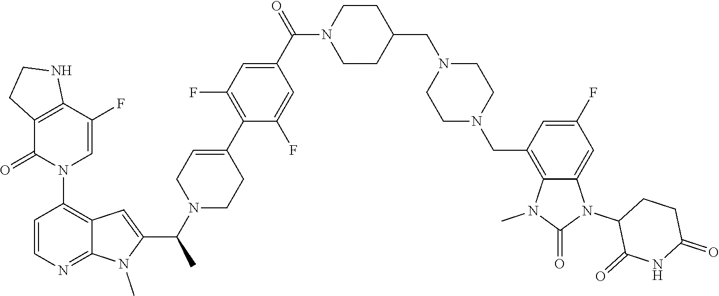 [SMILES: C[C@@H](c1cc2c(-n3cc(F)c4c(c3=O)CCN4)ccnc2n1C)N1CC=C(c2c(F)cc(C(=O)N3CCC(CN4CCN(Cc5cc(F)cc6c5n(C)c(=O)n6C5CCC(=O)NC5=O)CC4)CC3)cc2F)CC1] |
| 3u27 and 3u28 | 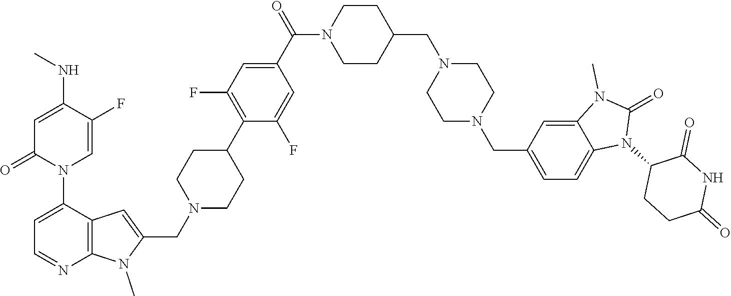 [SMILES: CNc1cc(=O)n(-c2ccnc3c2cc(CN2CCC(c4c(F)cc(C(=O)N5CCC(CN6CCN(Cc7ccc8c(c7)n(C)c(=O)n8[C@H]7CCC(=O)NC7=O)CC6)CC5)cc4F)CC2)n3C)cc1F] |
| | 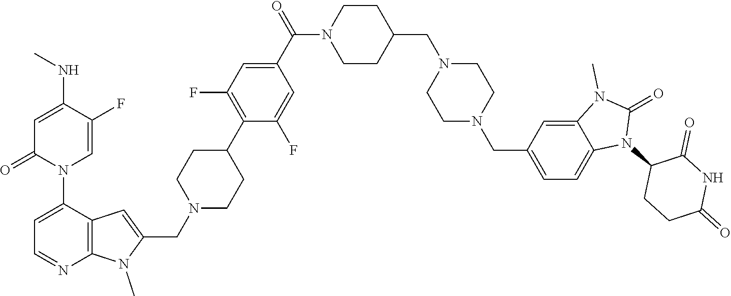 [SMILES: CNc1cc(=O)n(-c2ccnc3c2cc(CN2CCC(c4c(F)cc(C(=O)N5CCC(CN6CCN(Cc7ccc8c(c7)n(C)c(=O)n8[C@@H]7CCC(=O)NC7=O)CC6)CC5)cc4F)CC2)n3C)cc1F] |
| 3u270 | 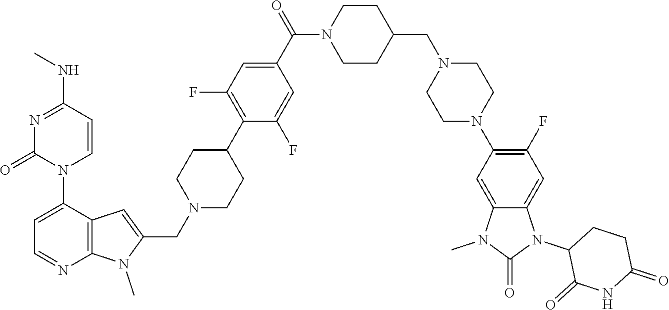 [SMILES: CNc1ccn(-c2ccnc3c2cc(CN2CCC(c4c(F)cc(C(=O)N5CCC(CN6CCN(c7cc8c(cc7F)n(C7CCC(=O)NC7=O)c(=O)n8C)CC6)CC5)cc4F)CC2)n3C)c(=O)n1] |
| 3u271 | 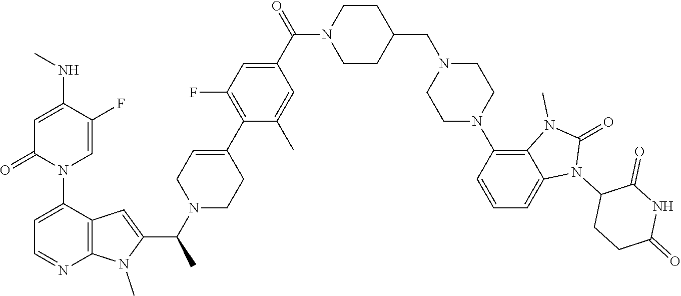 [SMILES: CNc1cc(=O)n(-c2ccnc3c2cc([C@H](C)N2CC=C(c4c(C)cc(C(=O)N5CCC(CN6CCN(c7cccc8c7n(C)c(=O)n8C7CCC(=O)NC7=O)CC6)CC5)cc4F)CC2)n3C)cc1F] |
| 3u272 | 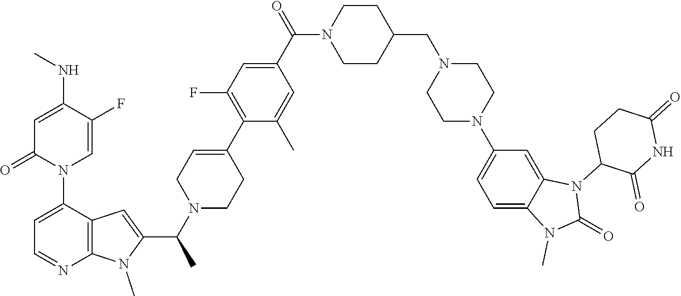 [SMILES: CNc1cc(=O)n(-c2ccnc3c2cc([C@H](C)N2CC=C(c4c(C)cc(C(=O)N5CCC(CN6CCN(c7ccc8c(c7)n(C7CCC(=O)NC7=O)c(=O)n8C)CC6)CC5)cc4F)CC2)n3C)cc1F] |
| 3u273 | 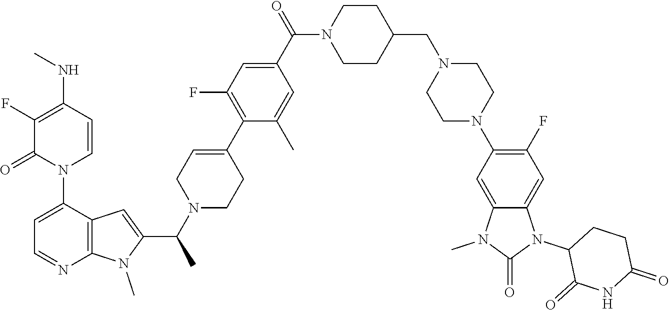 [SMILES: CNc1ccn(-c2ccnc3c2cc([C@H](C)N2CC=C(c4c(C)cc(C(=O)N5CCC(CN6CCN(c7cc8c(cc7F)n(C7CCC(=O)NC7=O)c(=O)n8C)CC6)CC5)cc4F)CC2)n3C)c(=O)c1F] |
| 3u274 | 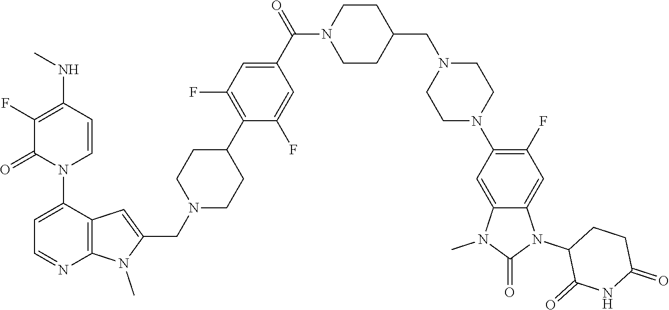 [SMILES: CNc1ccn(-c2ccnc3c2cc(CN2CCC(c4c(F)cc(C(=O)N5CCC(CN6CCN(c7cc8c(cc7F)n(C7CCC(=O)NC7=O)c(=O)n8C)CC6)CC5)cc4F)CC2)n3C)c(=O)c1F] |
| 3u275 | 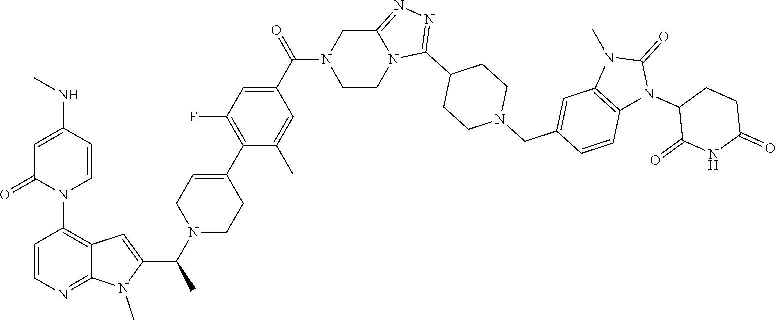 [SMILES: CNc1ccn(-c2ccnc3c2cc([C@H](C)N2CC=C(c4c(C)cc(C(=O)N5CCn6c(nnc6C6CCN(Cc7ccc8c(c7)n(C)c(=O)n8C7CCC(=O)NC7=O)CC6)C5)cc4F)CC2)n3C)c(=O)c1] |
| 3u276 | 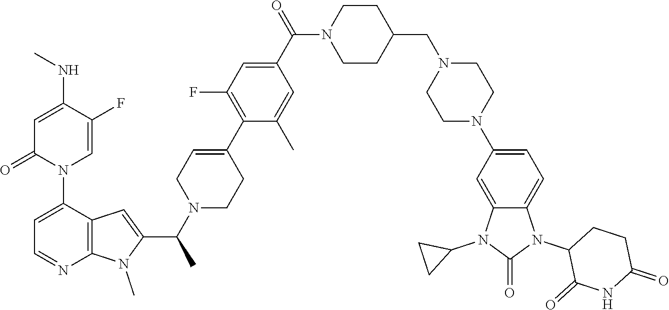 [SMILES: CNc1cc(=O)n(-c2ccnc3c2cc([C@H](C)N2CC=C(c4c(C)cc(C(=O)N5CCC(CN6CCN(c7ccc8c(c7)n(C7CC7)c(=O)n8C7CCC(=O)NC7=O)CC6)CC5)cc4F)CC2)n3C)cc1F] |
| 3u277 | 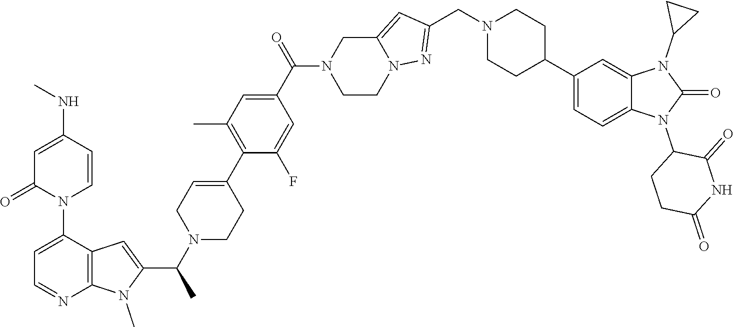 [SMILES: CNc1ccn(-c2ccnc3c2cc([C@H](C)N2CC=C(c4c(C)cc(C(=O)N5CCn6nc(CN7CCC(c8ccc9c(c8)n(C8CC8)c(=O)n9C8CCC(=O)NC8=O)CC7)cc6C5)cc4F)CC2)n3C)c(=O)c1] |
| 3u278 and 3u279 | 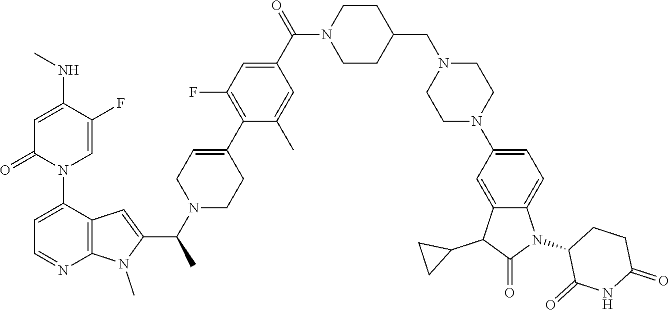 [SMILES: CNc1cc(=O)n(-c2ccnc3c2cc([C@H](C)N2CC=C(c4c(C)cc(C(=O)N5CCC(CN6CCN(c7ccc8c(c7)C(C7CC7)C(=O)N8[C@@H]7CCC(=O)NC7=O)CC6)CC5)cc4F)CC2)n3C)cc1F] |
| | 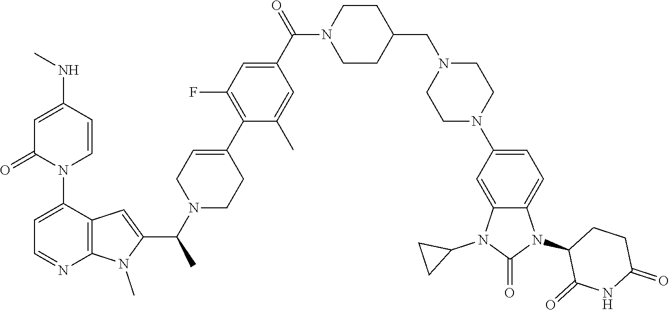 [SMILES: CNc1ccn(-c2ccnc3c2cc([C@H](C)N2CC=C(c4c(C)cc(C(=O)N5CCC(CN6CCN(c7ccc8c(c7)n(C7CC7)c(=O)n8[C@H]7CCC(=O)NC7=O)CC6)CC5)cc4F)CC2)n3C)c(=O)c1] |
| 3u280 and 3u281 | 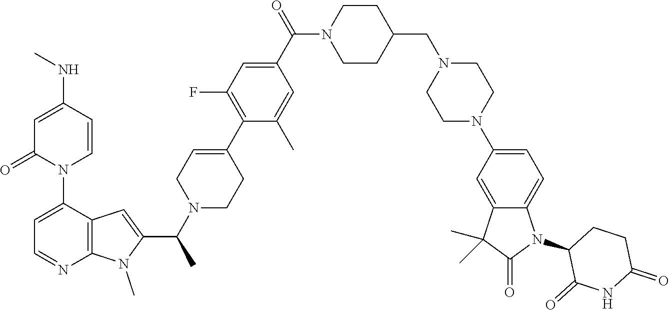 [SMILES: CNc1ccn(-c2ccnc3c2cc([C@H](C)N2CC=C(c4c(C)cc(C(=O)N5CCC(CN6CCN(c7ccc8c(c7)C(C)(C)C(=O)N8[C@H]7CCC(=O)NC7=O)CC6)CC5)cc4F)CC2)n3C)c(=O)c1] |
| | 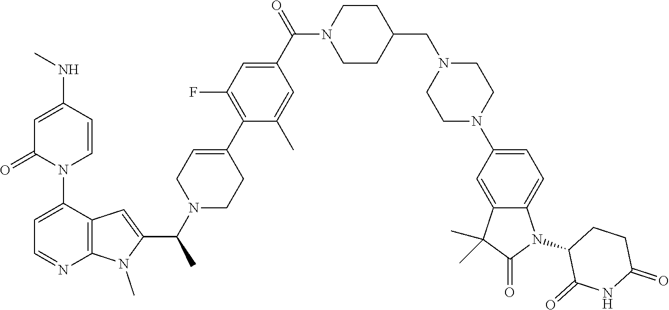 [SMILES: CNc1ccn(-c2ccnc3c2cc([C@H](C)N2CC=C(c4c(C)cc(C(=O)N5CCC(CN6CCN(c7ccc8c(c7)C(C)(C)C(=O)N8[C@@H]7CCC(=O)NC7=O)CC6)CC5)cc4F)CC2)n3C)c(=O)c1] |
| 3u282 | 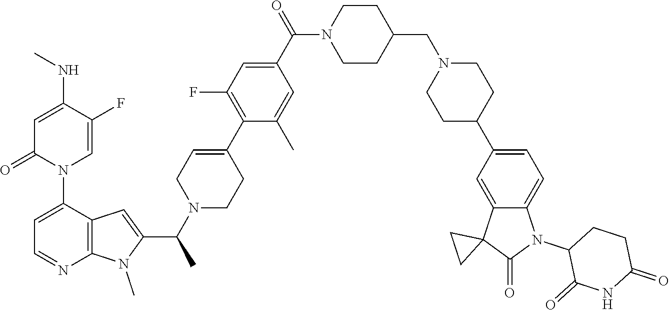 [SMILES: CNc1cc(=O)n(-c2ccnc3c2cc([C@H](C)N2CC=C(c4c(C)cc(C(=O)N5CCC(CN6CCC(c7ccc8c(c7)C7(CC7)C(=O)N8C7CCC(=O)NC7=O)CC6)CC5)cc4F)CC2)n3C)cc1F] |
| 3u283 | 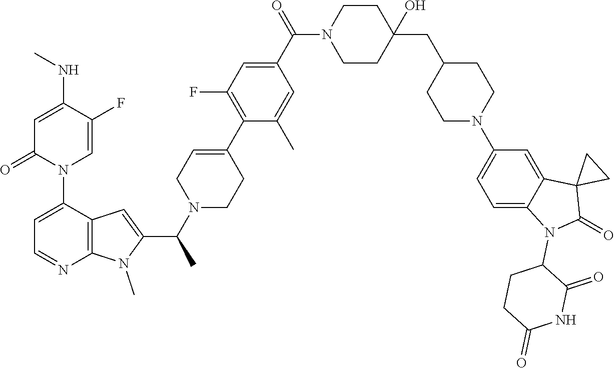 [SMILES: CNc1cc(=O)n(-c2ccnc3c2cc([C@H](C)N2CC=C(c4c(C)cc(C(=O)N5CCC(O)(CC6CCN(c7ccc8c(c7)C7(CC7)C(=O)N8C7CCC(=O)NC7=O)CC6)CC5)cc4F)CC2)n3C)cc1F] |
| 3u284 | 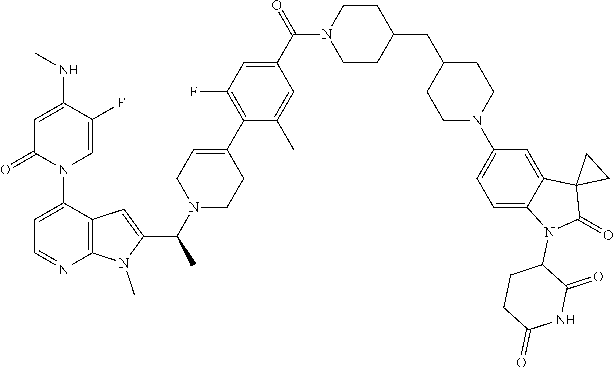 [SMILES: CNc1cc(=O)n(-c2ccnc3c2cc([C@H](C)N2CC=C(c4c(C)cc(C(=O)N5CCC(CC6CCN(c7ccc8c(c7)C7(CC7)C(=O)N8C7CCC(=O)NC7=O)CC6)CC5)cc4F)CC2)n3C)cc1F] |
| 3u285 and 3u286 | 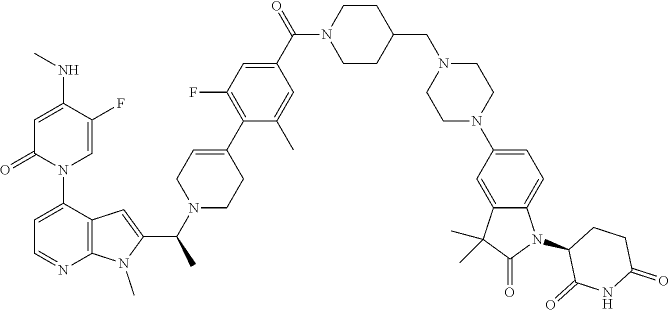 [SMILES: CNc1cc(=O)n(-c2ccnc3c2cc([C@H](C)N2CC=C(c4c(C)cc(C(=O)N5CCC(CN6CCN(c7ccc8c(c7)C(C)(C)C(=O)N8[C@H]7CCC(=O)NC7=O)CC6)CC5)cc4F)CC2)n3C)cc1F] |
| | 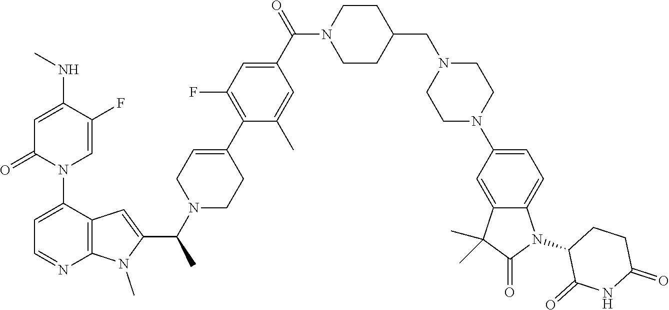 [SMILES: CNc1cc(=O)n(-c2ccnc3c2cc([C@H](C)N2CC=C(c4c(C)cc(C(=O)N5CCC(CN6CCN(c7ccc8c(c7)C(C)(C)C(=O)N8[C@@H]7CCC(=O)NC7=O)CC6)CC5)cc4F)CC2)n3C)cc1F] |
| 3u287 | 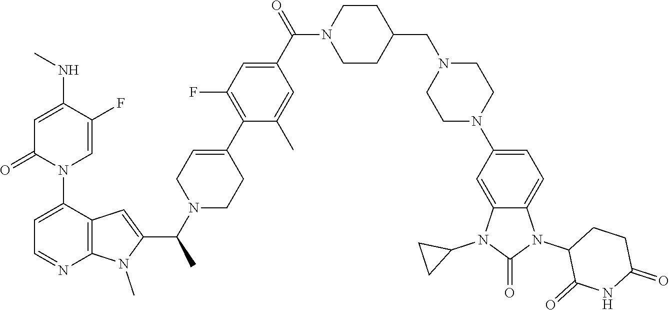 [SMILES: CNc1cc(=O)n(-c2ccnc3c2cc([C@H](C)N2CC=C(c4c(C)cc(C(=O)N5CCC(CN6CCN(c7ccc8c(c7)n(C7CC7)c(=O)n8C7CCC(=O)NC7=O)CC6)CC5)cc4F)CC2)n3C)cc1F] |
| 3u288 and 3u289 | 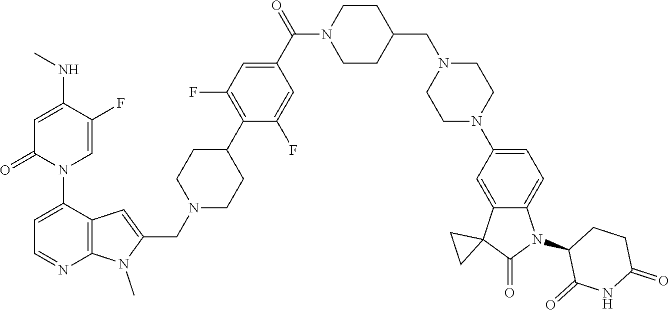 [SMILES: CNc1cc(=O)n(-c2ccnc3c2cc(CN2CCC(c4c(F)cc(C(=O)N5CCC(CN6CCN(c7ccc8c(c7)C7(CC7)C(=O)N8[C@H]7CCC(=O)NC7=O)CC6)CC5)cc4F)CC2)n3C)cc1F] |
| | 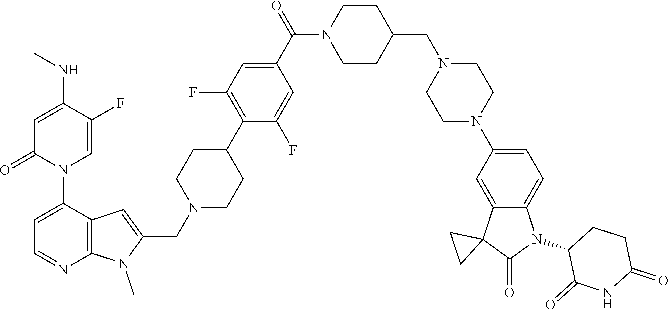 [SMILES: CNc1cc(=O)n(-c2ccnc3c2cc(CN2CCC(c4c(F)cc(C(=O)N5CCC(CN6CCN(c7ccc8c(c7)C7(CC7)C(=O)N8[C@@H]7CCC(=O)NC7=O)CC6)CC5)cc4F)CC2)n3C)cc1F] |
| 3u29 | 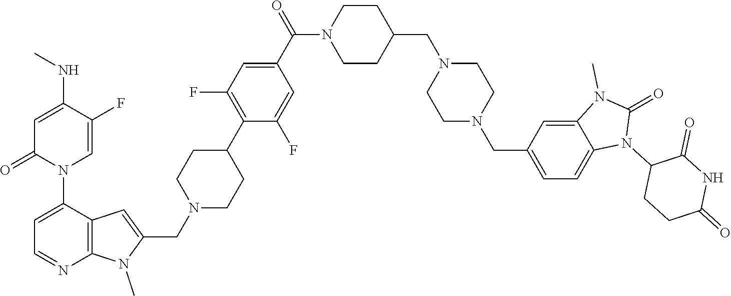 [SMILES: CNc1cc(=O)n(-c2ccnc3c2cc(CN2CCC(c4c(F)cc(C(=O)N5CCC(CN6CCN(Cc7ccc8c(c7)n(C)c(=O)n8C7CCC(=O)NC7=O)CC6)CC5)cc4F)CC2)n3C)cc1F] |
| 3u290 | 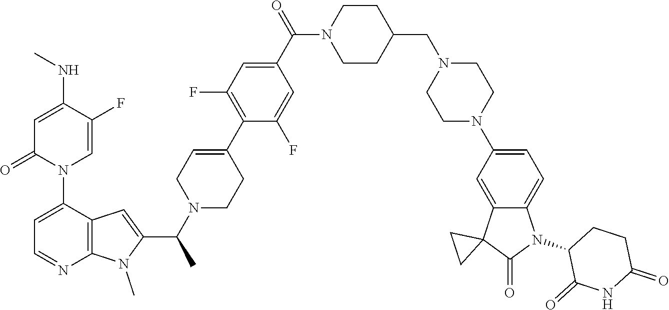 [SMILES: CNc1cc(=O)n(-c2ccnc3c2cc([C@H](C)N2CC=C(c4c(F)cc(C(=O)N5CCC(CN6CCN(c7ccc8c(c7)C7(CC7)C(=O)N8[C@@H]7CCC(=O)NC7=O)CC6)CC5)cc4F)CC2)n3C)cc1F] |
| | or |
| | 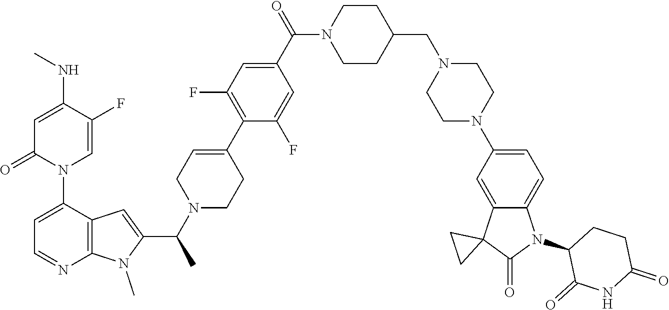 [SMILES: CNc1cc(=O)n(-c2ccnc3c2cc([C@H](C)N2CC=C(c4c(F)cc(C(=O)N5CCC(CN6CCN(c7ccc8c(c7)C7(CC7)C(=O)N8[C@H]7CCC(=O)NC7=O)CC6)CC5)cc4F)CC2)n3C)cc1F] |
| 3u291 and 3u292 | 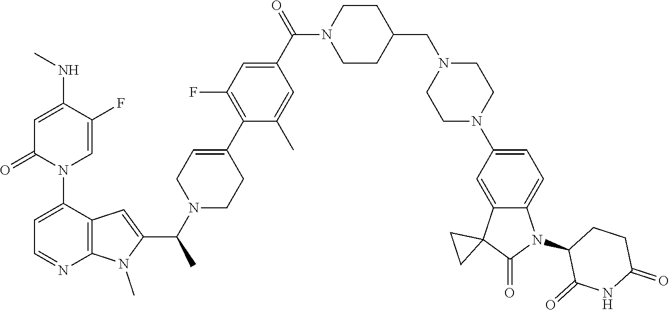 [SMILES: CNc1cc(=O)n(-c2ccnc3c2cc([C@H](C)N2CC=C(c4c(C)cc(C(=O)N5CCC(CN6CCN(c7ccc8c(c7)C7(CC7)C(=O)N8[C@H]7CCC(=O)NC7=O)CC6)CC5)cc4F)CC2)n3C)cc1F] |
| | 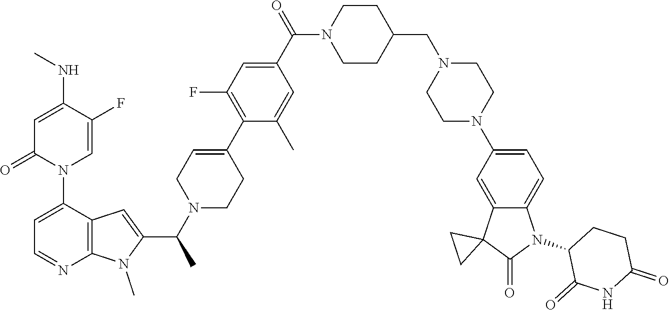 [SMILES: CNc1cc(=O)n(-c2ccnc3c2cc([C@H](C)N2CC=C(c4c(C)cc(C(=O)N5CCC(CN6CCN(c7ccc8c(c7)C7(CC7)C(=O)N8[C@@H]7CCC(=O)NC7=O)CC6)CC5)cc4F)CC2)n3C)cc1F] |
| 3u293 and 3u294 | 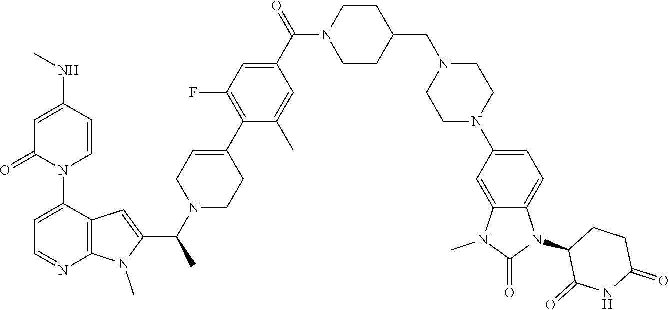 [SMILES: CNc1ccn(-c2ccnc3c2cc([C@H](C)N2CC=C(c4c(C)cc(C(=O)N5CCC(CN6CCN(c7ccc8c(c7)n(C)c(=O)n8[C@H]7CCC(=O)NC7=O)CC6)CC5)cc4F)CC2)n3C)c(=O)c1] |
| | 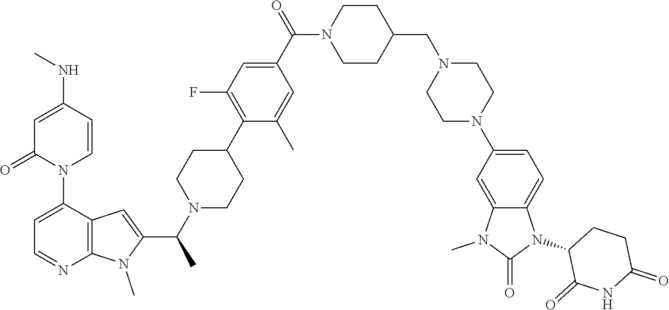 [SMILES: CNc1ccn(-c2ccnc3c2cc([C@H](C)N2CCC(c4c(C)cc(C(=O)N5CCC(CN6CCN(c7ccc8c(c7)n(C)c(=O)n8[C@@H]7CCC(=O)NC7=O)CC6)CC5)cc4F)CC2)n3C)c(=O)c1] |
| 3u295 | 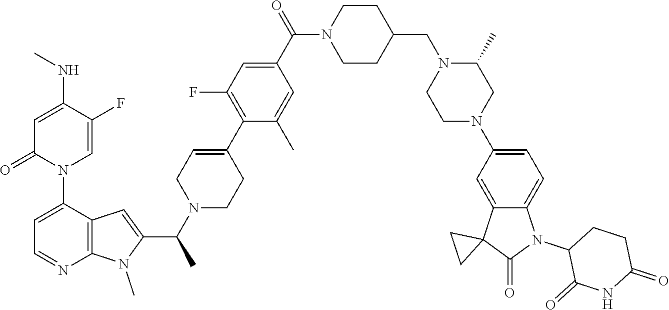 [SMILES: CNc1cc(=O)n(-c2ccnc3c2cc([C@H](C)N2CC=C(c4c(C)cc(C(=O)N5CCC(CN6CCN(c7ccc8c(c7)C7(CC7)C(=O)N8C7CCC(=O)NC7=O)C[C@H]6C)CC5)cc4F)CC2)n3C)cc1F] |
| 3u296 and 3u297 | 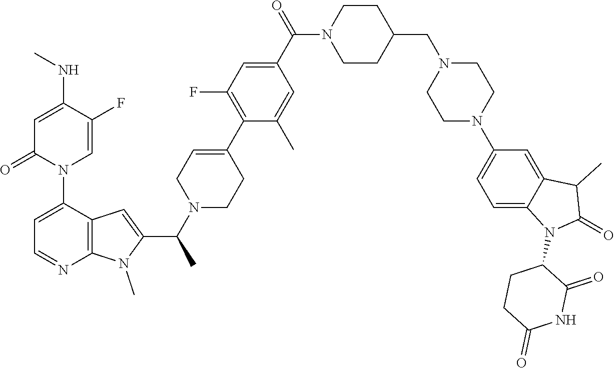 [SMILES: CNc1cc(=O)n(-c2ccnc3c2cc([C@H](C)N2CC=C(c4c(C)cc(C(=O)N5CCC(CN6CCN(c7ccc8c(c7)C(C)C(=O)N8[C@H]7CCC(=O)NC7=O)CC6)CC5)cc4F)CC2)n3C)cc1F] |
| | 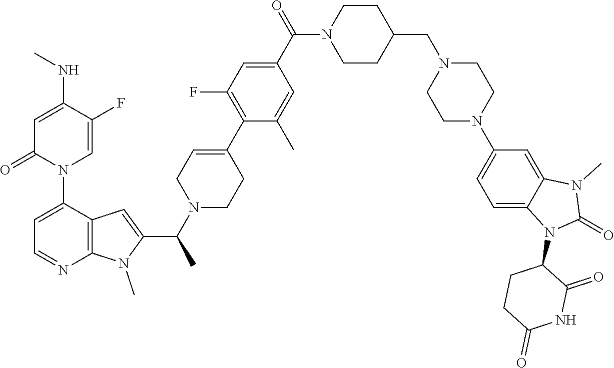 [SMILES: CNc1cc(=O)n(-c2ccnc3c2cc([C@H](C)N2CC=C(c4c(C)cc(C(=O)N5CCC(CN6CCN(c7ccc8c(c7)n(C)c(=O)n8[C@@H]7CCC(=O)NC7=O)CC6)CC5)cc4F)CC2)n3C)cc1F] |
| 3u298 | 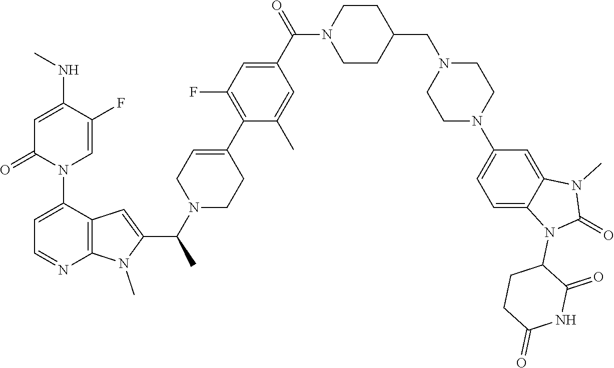 [SMILES: CNc1cc(=O)n(-c2ccnc3c2cc([C@H](C)N2CC=C(c4c(C)cc(C(=O)N5CCC(CN6CCN(c7ccc8c(c7)n(C)c(=O)n8C7CCC(=O)NC7=O)CC6)CC5)cc4F)CC2)n3C)cc1F] |
| 3u299 | 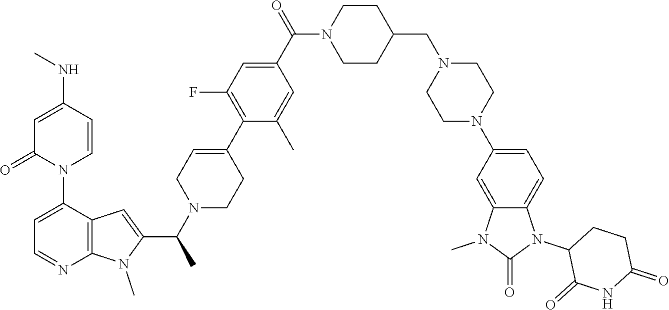 [SMILES: CNc1ccn(-c2ccnc3c2cc([C@H](C)N2CC=C(c4c(C)cc(C(=O)N5CCC(CN6CCN(c7ccc8c(c7)n(C)c(=O)n8C7CCC(=O)NC7=O)CC6)CC5)cc4F)CC2)n3C)c(=O)c1] |
| 3u30 and 3u31 | 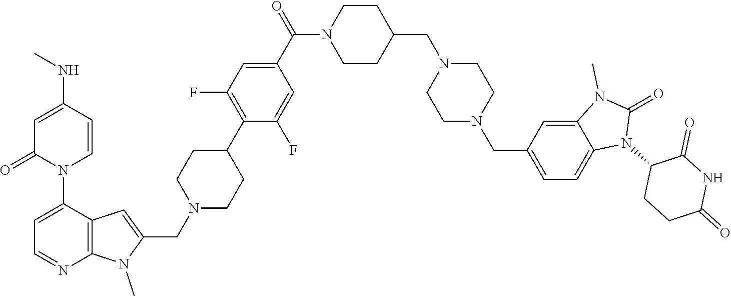 [SMILES: CNc1ccn(-c2ccnc3c2cc(CN2CCC(c4c(F)cc(C(=O)N5CCC(CN6CCN(Cc7ccc8c(c7)n(C)c(=O)n8[C@H]7CCC(=O)NC7=O)CC6)CC5)cc4F)CC2)n3C)c(=O)c1] |
| | 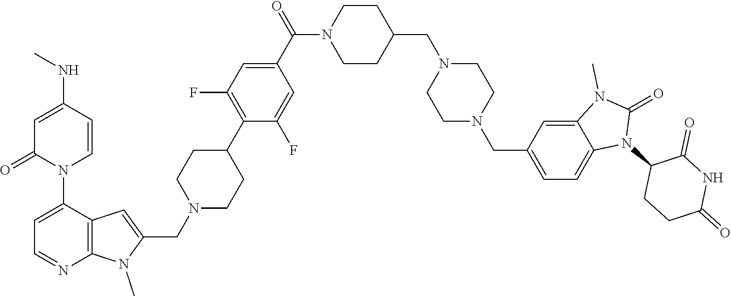 [SMILES: CNc1ccn(-c2ccnc3c2cc(CN2CCC(c4c(F)cc(C(=O)N5CCC(CN6CCN(Cc7ccc8c(c7)n(C)c(=O)n8[C@@H]7CCC(=O)NC7=O)CC6)CC5)cc4F)CC2)n3C)c(=O)c1] |
| 3u300 | 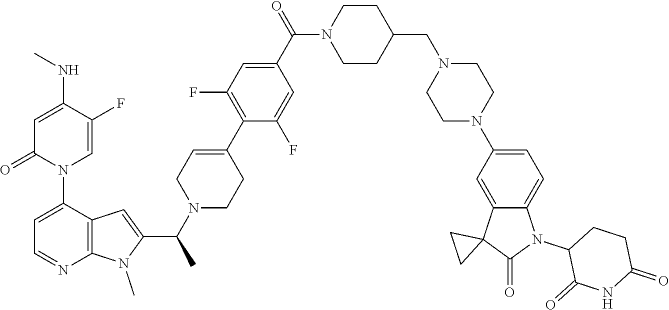 [SMILES: CNc1cc(=O)n(-c2ccnc3c2cc([C@H](C)N2CC=C(c4c(F)cc(C(=O)N5CCC(CN6CCN(c7ccc8c(c7)C7(CC7)C(=O)N8C7CCC(=O)NC7=O)CC6)CC5)cc4F)CC2)n3C)cc1F] |
| 3u301 | 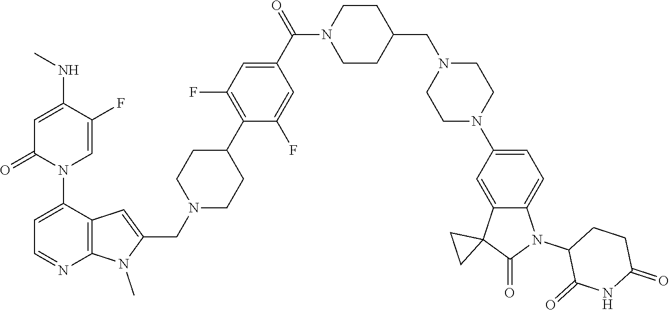 [SMILES: CNc1cc(=O)n(-c2ccnc3c2cc(CN2CCC(c4c(F)cc(C(=O)N5CCC(CN6CCN(c7ccc8c(c7)C7(CC7)C(=O)N8C7CCC(=O)NC7=O)CC6)CC5)cc4F)CC2)n3C)cc1F] |
| 3u302 | 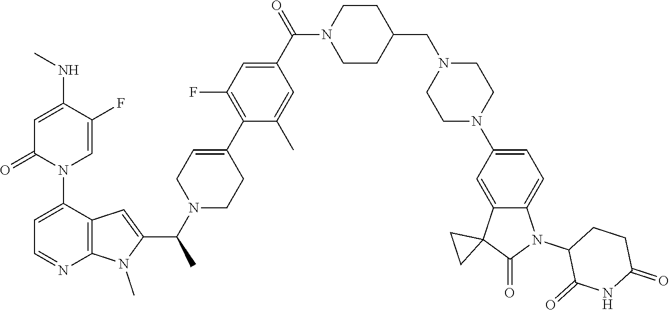 [SMILES: CNc1cc(=O)n(-c2ccnc3c2cc([C@H](C)N2CC=C(c4c(C)cc(C(=O)N5CCC(CN6CCN(c7ccc8c(c7)C7(CC7)C(=O)N8C7CCC(=O)NC7=O)CC6)CC5)cc4F)CC2)n3C)cc1F] |
| 3u303 | 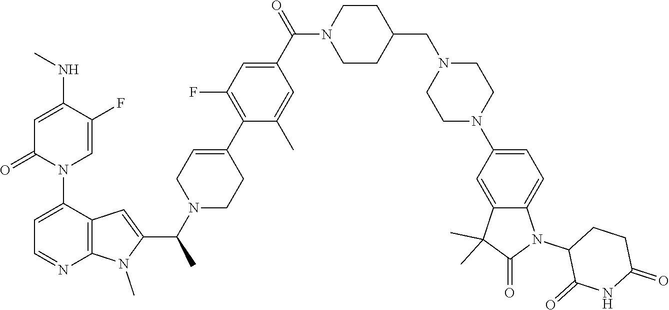 [SMILES: CNc1cc(=O)n(-c2ccnc3c2cc([C@H](C)N2CC=C(c4c(C)cc(C(=O)N5CCC(CN6CCN(c7ccc8c(c7)C(C)(C)C(=O)N8C7CCC(=O)NC7=O)CC6)CC5)cc4F)CC2)n3C)cc1F] |
| 3u304 | 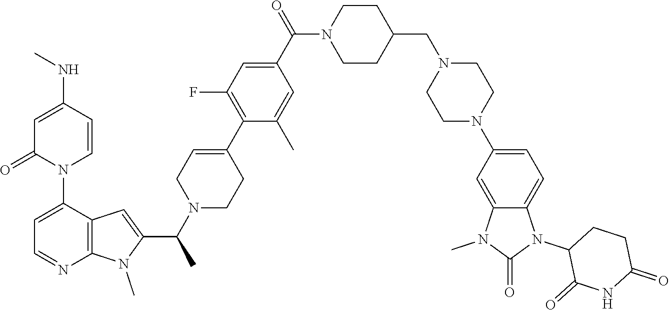 [SMILES: CNc1ccn(-c2ccnc3c2cc([C@H](C)N2CC=C(c4c(C)cc(C(=O)N5CCC(CN6CCN(c7ccc8c(c7)n(C)c(=O)n8C7CCC(=O)NC7=O)CC6)CC5)cc4F)CC2)n3C)c(=O)c1] |
| 3u305 | 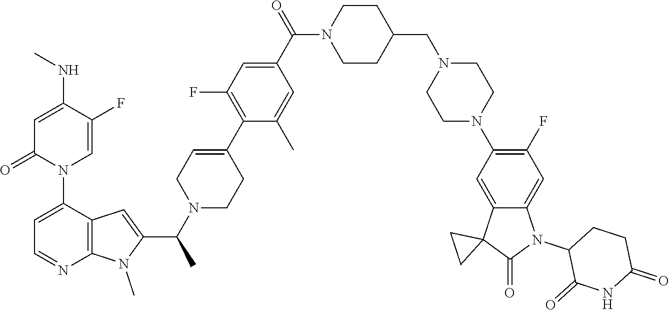 [SMILES: CNc1cc(=O)n(-c2ccnc3c2cc([C@H](C)N2CC=C(c4c(C)cc(C(=O)N5CCC(CN6CCN(c7cc8c(cc7F)N(C7CCC(=O)NC7=O)C(=O)C87CC7)CC6)CC5)cc4F)CC2)n3C)cc1F] |
| 3u306 | 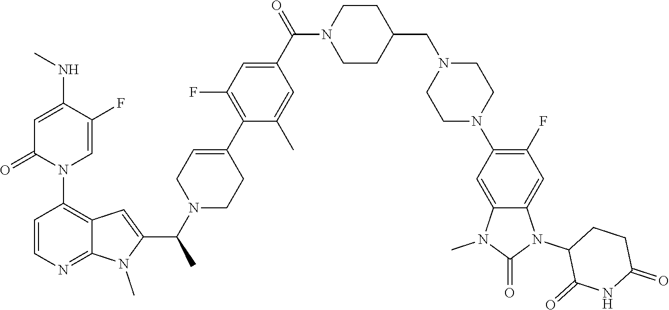 [SMILES: CNc1cc(=O)n(-c2ccnc3c2cc([C@H](C)N2CC=C(c4c(C)cc(C(=O)N5CCC(CN6CCN(c7cc8c(cc7F)n(C7CCC(=O)NC7=O)c(=O)n8C)CC6)CC5)cc4F)CC2)n3C)cc1F] |
| 3u307 and 3u308 | 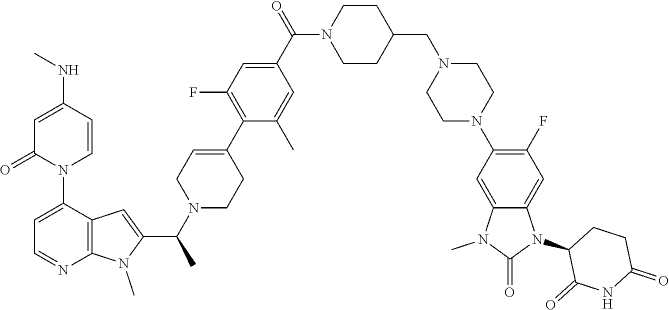 [SMILES: CNc1ccn(-c2ccnc3c2cc([C@H](C)N2CC=C(c4c(C)cc(C(=O)N5CCC(CN6CCN(c7cc8c(cc7F)n([C@H]7CCC(=O)NC7=O)c(=O)n8C)CC6)CC5)cc4F)CC2)n3C)c(=O)c1] |
| | 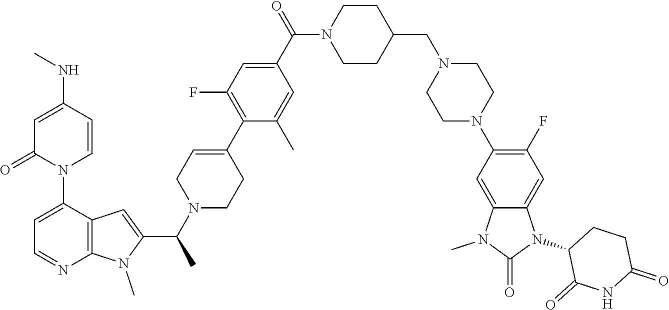 [SMILES: CNc1ccn(-c2ccnc3c2cc([C@H](C)N2CC=C(c4c(C)cc(C(=O)N5CCC(CN6CCN(c7cc8c(cc7F)n([C@@H]7CCC(=O)NC7=O)c(=O)n8C)CC6)CC5)cc4F)CC2)n3C)c(=O)c1] |
| 3u309 | 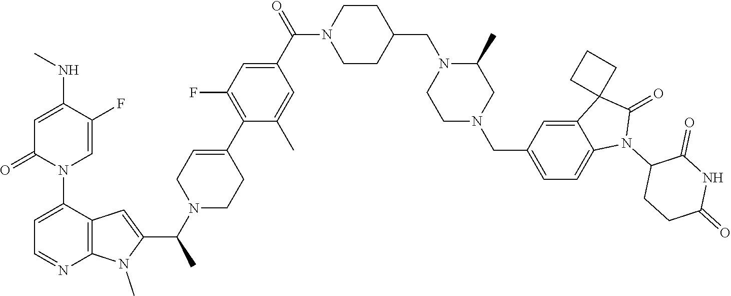 [SMILES: CNc1cc(=O)n(-c2ccnc3c2cc([C@H](C)N2CC=C(c4c(C)cc(C(=O)N5CCC(CN6CCN(Cc7ccc8c(c7)C7(CCC7)C(=O)N8C7CCC(=O)NC7=O)C[C@@H]6C)CC5)cc4F)CC2)n3C)cc1F] |
| 3u310 | 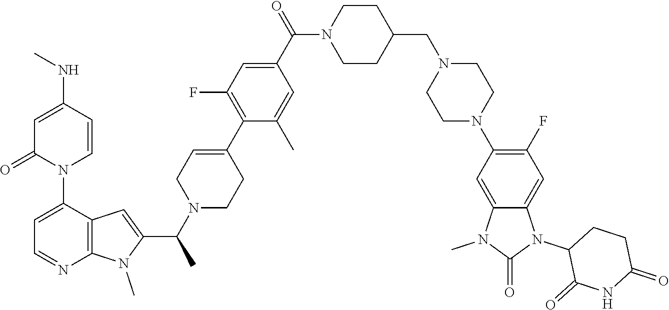 [SMILES: CNc1ccn(-c2ccnc3c2cc([C@H](C)N2CC=C(c4c(C)cc(C(=O)N5CCC(CN6CCN(c7cc8c(cc7F)n(C7CCC(=O)NC7=O)c(=O)n8C)CC6)CC5)cc4F)CC2)n3C)c(=O)c1] |
| 3u311 | 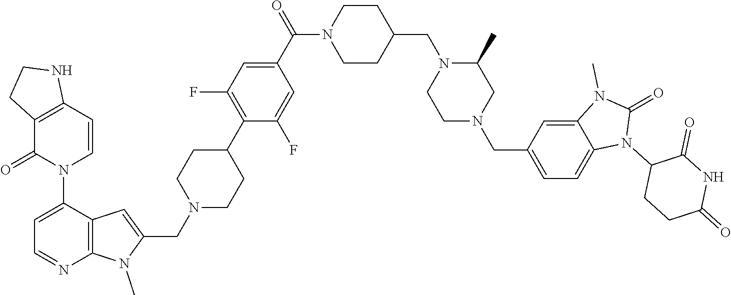 [SMILES: C[C@H]1CN(Cc2ccc3c(c2)n(C)c(=O)n3C2CCC(=O)NC2=O)CCN1CC1CCN(C(=O)c2cc(F)c(C3CCN(Cc4cc5c(-n6ccc7c(c6=O)CCN7)ccnc5n4C)CC3)c(F)c2)CC1] |
| 3u32 | 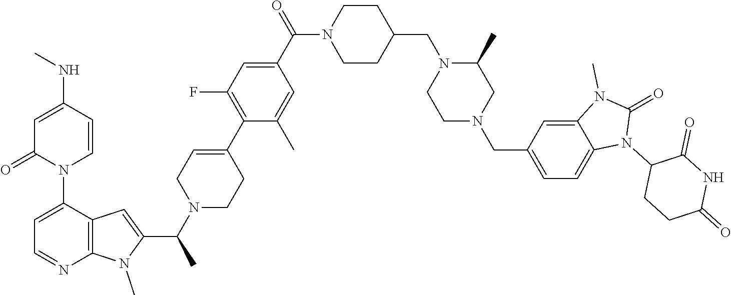 [SMILES: CNc1ccn(-c2ccnc3c2cc([C@H](C)N2CC=C(c4c(C)cc(C(=O)N5CCC(CN6CCN(Cc7ccc8c(c7)n(C)c(=O)n8C7CCC(=O)NC7=O)C[C@@H]6C)CC5)cc4F)CC2)n3C)c(=O)c1] |
| 3u33 and 3u34 | 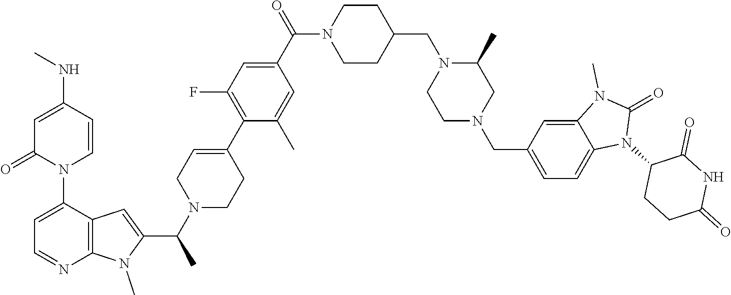 [SMILES: CNc1ccn(-c2ccnc3c2cc([C@H](C)N2CC=C(c4c(C)cc(C(=O)N5CCC(CN6CCN(Cc7ccc8c(c7)n(C)c(=O)n8[C@H]7CCC(=O)NC7=O)C[C@@H]6C)CC5)cc4F)CC2)n3C)c(=O)c1] |
| | 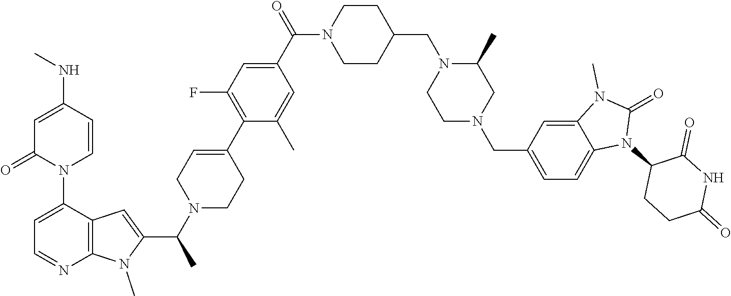 [SMILES: CNc1ccn(-c2ccnc3c2cc([C@H](C)N2CC=C(c4c(C)cc(C(=O)N5CCC(CN6CCN(Cc7ccc8c(c7)n(C)c(=O)n8[C@@H]7CCC(=O)NC7=O)C[C@@H]6C)CC5)cc4F)CC2)n3C)c(=O)c1] |
| 3u35 | 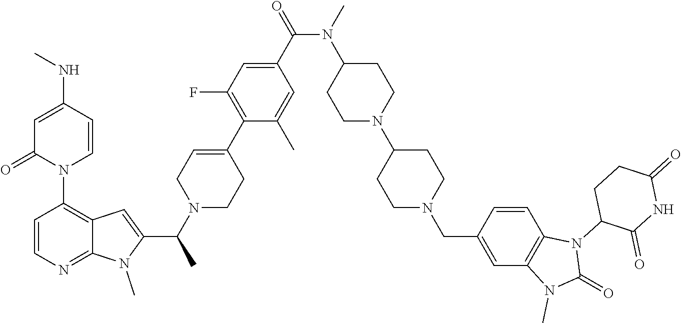 [SMILES: CNc1ccn(-c2ccnc3c2cc([C@H](C)N2CC=C(c4c(C)cc(C(=O)N(C)C5CCN(C6CCN(Cc7ccc8c(c7)n(C)c(=O)n8C7CCC(=O)NC7=O)CC6)CC5)cc4F)CC2)n3C)c(=O)c1] |
| 3u36 | 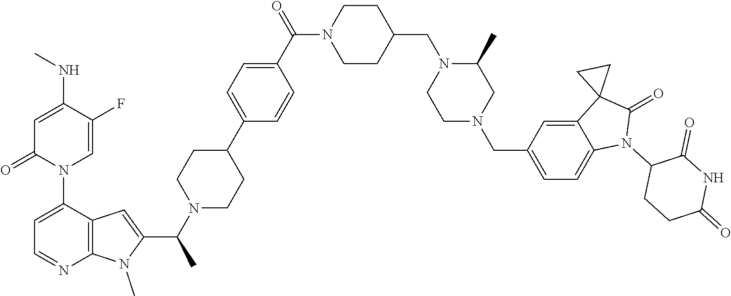 [SMILES: CNc1cc(=O)n(-c2ccnc3c2cc([C@H](C)N2CCC(c4ccc(C(=O)N5CCC(CN6CCN(Cc7ccc8c(c7)C7(CC7)C(=O)N8C7CCC(=O)NC7=O)C[C@@H]6C)CC5)cc4)CC2)n3C)cc1F] |
| 3u37 and 3u38 | 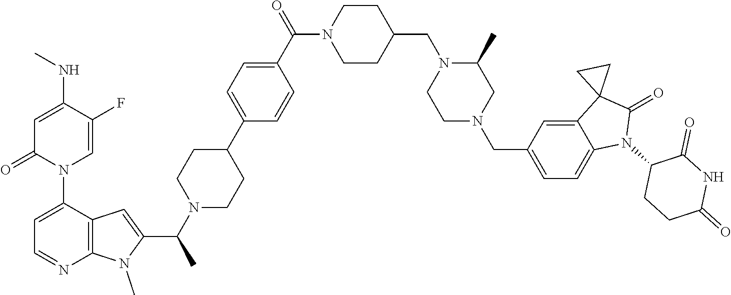 [SMILES: CNc1cc(=O)n(-c2ccnc3c2cc([C@H](C)N2CCC(c4ccc(C(=O)N5CCC(CN6CCN(Cc7ccc8c(c7)C7(CC7)C(=O)N8[C@H]7CCC(=O)NC7=O)C[C@@H]6C)CC5)cc4)CC2)n3C)cc1F] |
| | 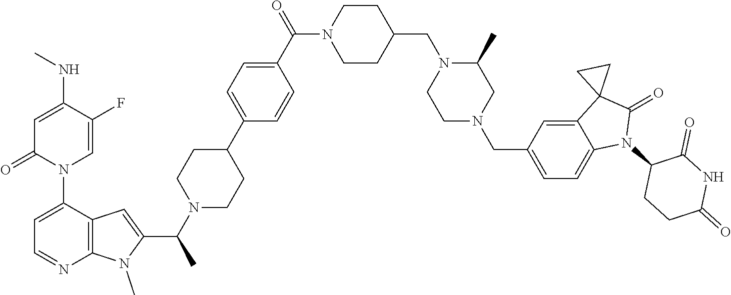 [SMILES: CNc1cc(=O)n(-c2ccnc3c2cc([C@H](C)N2CCC(c4ccc(C(=O)N5CCC(CN6CCN(Cc7ccc8c(c7)C7(CC7)C(=O)N8[C@@H]7CCC(=O)NC7=O)C[C@@H]6C)CC5)cc4)CC2)n3C)cc1F] |
| 3u39 | 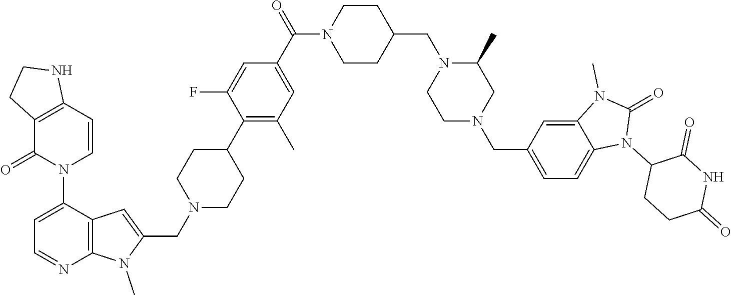 [SMILES: Cc1cc(C(=O)N2CCC(CN3CCN(Cc4ccc5c(c4)n(C)c(=O)n5C4CCC(=O)NC4=O)C[C@@H]3C)CC2)cc(F)c1C1CCN(Cc2cc3c(-n4ccc5c(c4=O)CCN5)ccnc3n2C)CC1] |
| 3u4 | 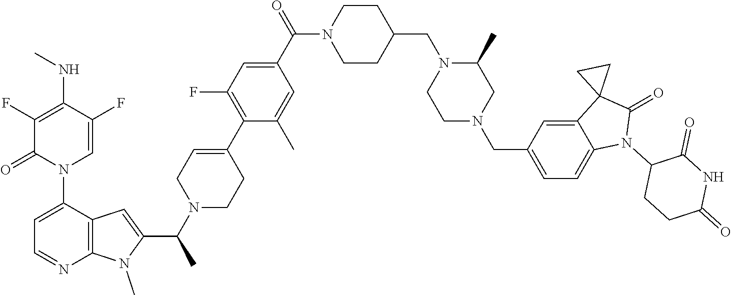 [SMILES: CNc1c(F)cn(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)c(=O)c1F] |
| 3u40 and 3u41 | 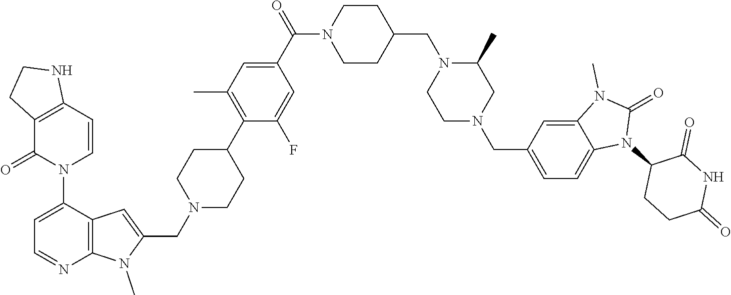 [SMILES: Cc1cc(C(=O)N2CCC(CN3CCN(Cc4ccc5c(c4)n(C)c(=O)n5[C@@H]4CCC(=O)NC4=O)C[C@@H]3C)CC2)cc(F)c1C1CCN(Cc2cc3c(-n4ccc5c(c4=O)CCN5)ccnc3n2C)CC1] |
| | 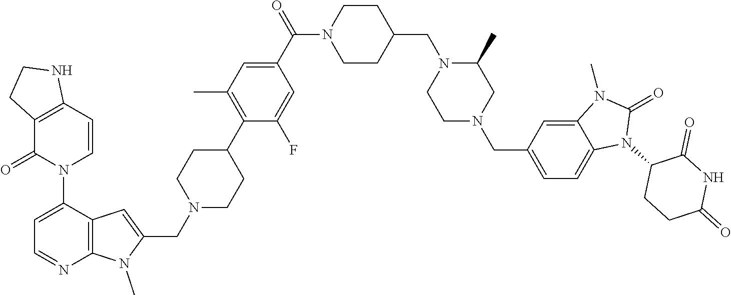 [SMILES: Cc1cc(C(=O)N2CCC(CN3CCN(Cc4ccc5c(c4)n(C)c(=O)n5[C@H]4CCC(=O)NC4=O)C[C@@H]3C)CC2)cc(F)c1C1CCN(Cc2cc3c(-n4ccc5c(c4=O)CCN5)ccnc3n2C)CC1] |
| 3u42 | 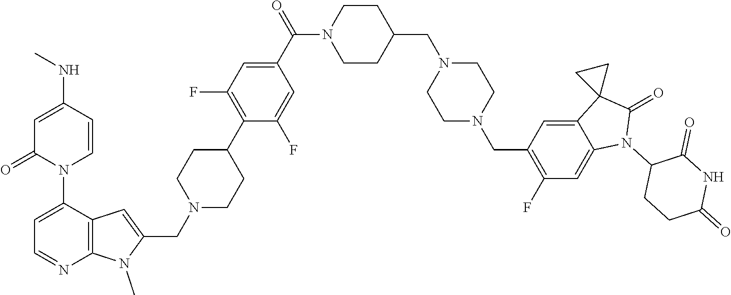 [SMILES: CNc1ccn(-c2ccnc3c2cc(CN2CCC(c4c(F)cc(C(=O)N5CCC(CN6CCN(Cc7cc8c(cc7F)N(C7CCC(=O)NC7=O)C(=O)C87CC7)CC6)CC5)cc4F)CC2)n3C)c(=O)c1] |
| 3u43 and 3u44 | 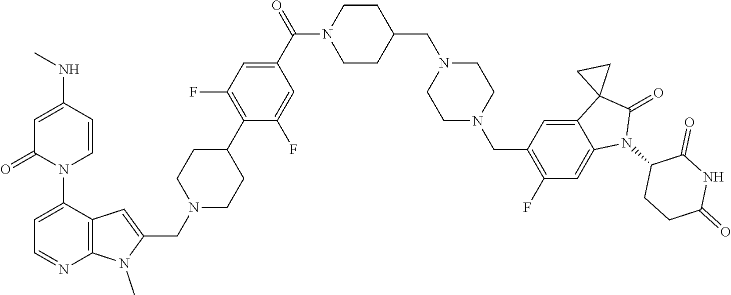 [SMILES: CNc1ccn(-c2ccnc3c2cc(CN2CCC(c4c(F)cc(C(=O)N5CCC(CN6CCN(Cc7cc8c(cc7F)N([C@H]7CCC(=O)NC7=O)C(=O)C87CC7)CC6)CC5)cc4F)CC2)n3C)c(=O)c1] |
| | 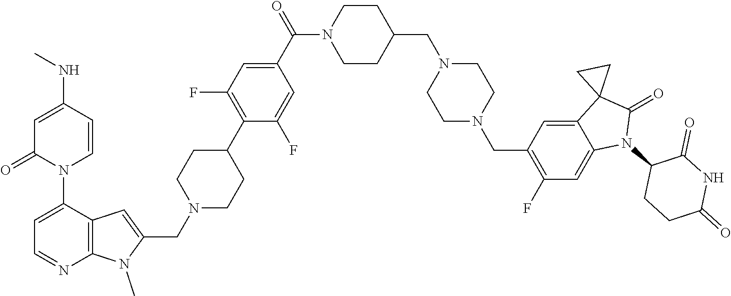 [SMILES: CNc1ccn(-c2ccnc3c2cc(CN2CCC(c4c(F)cc(C(=O)N5CCC(CN6CCN(Cc7cc8c(cc7F)N([C@@H]7CCC(=O)NC7=O)C(=O)C87CC7)CC6)CC5)cc4F)CC2)n3C)c(=O)c1] |
| 3u45 | 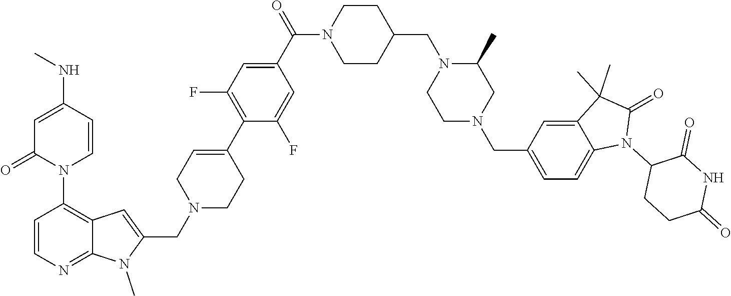 [SMILES: CNc1ccn(-c2ccnc3c2cc(CN2CC=C(c4c(F)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)c(=O)c1] |
| 3u46 and 3u47 | 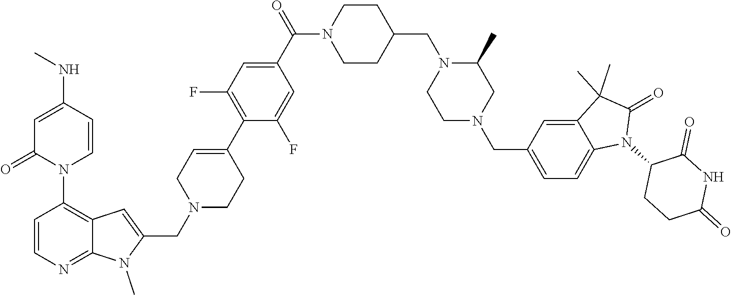 [SMILES: CNc1ccn(-c2ccnc3c2cc(CN2CC=C(c4c(F)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)c(=O)c1] |
| | 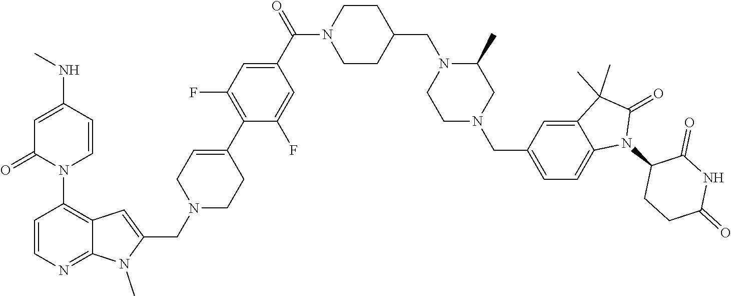 [SMILES: CNc1ccn(-c2ccnc3c2cc(CN2CC=C(c4c(F)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)c(=O)c1] |
| 3u48 | 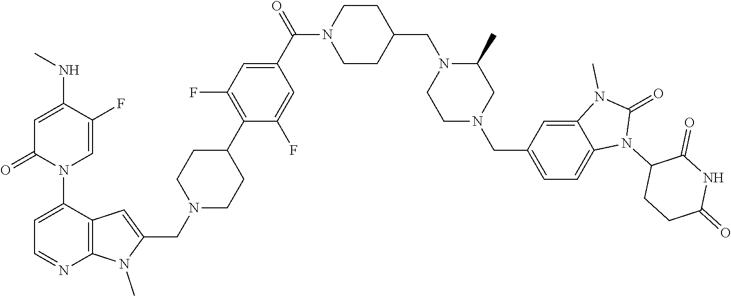 [SMILES: CNc1cc(=O)n(-c2ccnc3c2cc(CN2CCC(c4c(F)cc(C(=O)N5CCC(CN6CCN(Cc7ccc8c(c7)n(C)c(=O)n8C7CCC(=O)NC7=O)C[C@@H]6C)CC5)cc4F)CC2)n3C)cc1F] |
| 3u49 and 3u50 | 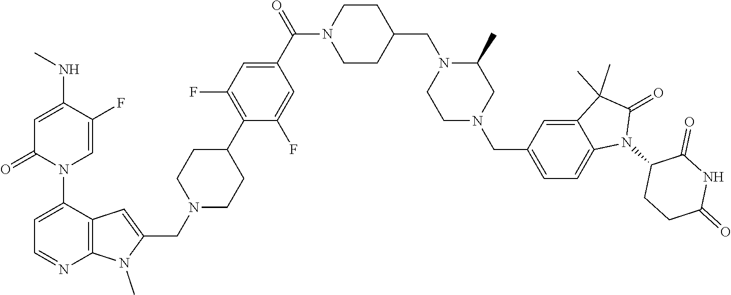 [SMILES: CNc1cc(=O)n(-c2ccnc3c2cc(CN2CCC(c4c(F)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)cc1F] |
| | 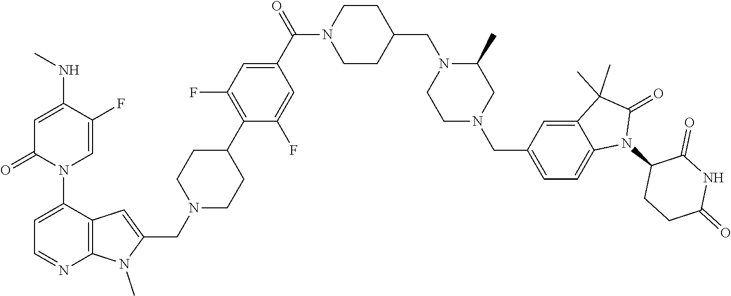 [SMILES: CNc1cc(=O)n(-c2ccnc3c2cc(CN2CCC(c4c(F)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)cc1F] |
| 3u5 | 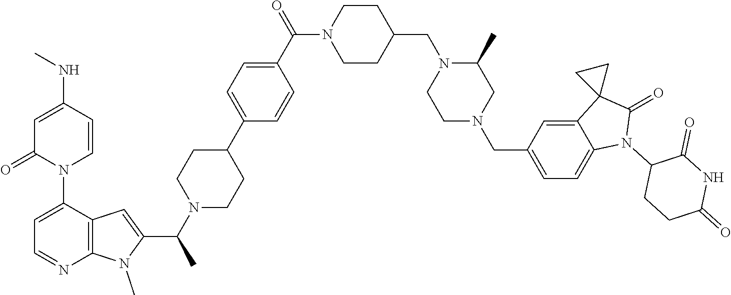 [SMILES: CNc1ccn(-c2ccnc3c2cc([C@H](C)N2CCC(c4ccc(C(=O)N5CCC(CN6CCN(Cc7ccc8c(c7)C7(CC7)C(=O)N8C7CCC(=O)NC7=O)C[C@@H]6C)CC5)cc4)CC2)n3C)c(=O)c1] |
| 3u51 | 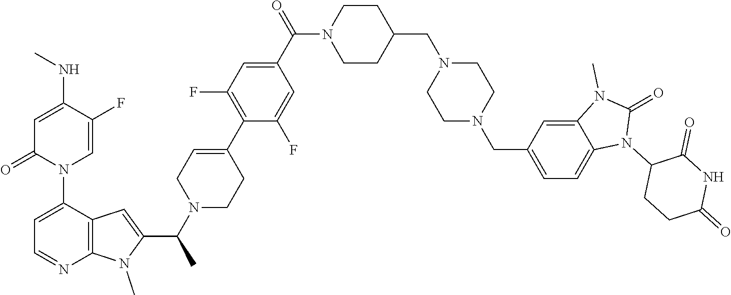 [SMILES: CNc1cc(=O)n(-c2ccnc3c2cc([C@H](C)N2CC=C(c4c(F)cc(C(=O)N5CCC(CN6CCN(Cc7ccc8c(c7)n(C)c(=O)n8C7CCC(=O)NC7=O)CC6)CC5)cc4F)CC2)n3C)cc1F] |
| 3u52 and 3u53 | 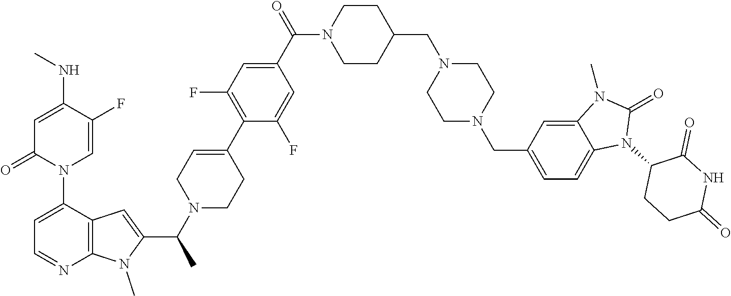 [SMILES: CNc1cc(=O)n(-c2ccnc3c2cc([C@H](C)N2CC=C(c4c(F)cc(C(=O)N5CCC(CN6CCN(Cc7ccc8c(c7)n(C)c(=O)n8[C@H]7CCC(=O)NC7=O)CC6)CC5)cc4F)CC2)n3C)cc1F] |
| | 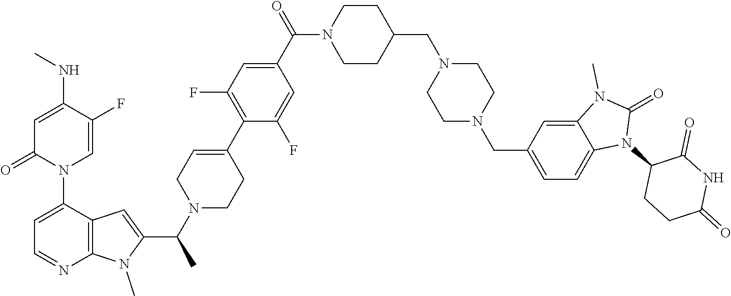 [SMILES: CNc1cc(=O)n(-c2ccnc3c2cc([C@H](C)N2CC=C(c4c(F)cc(C(=O)N5CCC(CN6CCN(Cc7ccc8c(c7)n(C)c(=O)n8[C@@H]7CCC(=O)NC7=O)CC6)CC5)cc4F)CC2)n3C)cc1F] |
| 3u54 | 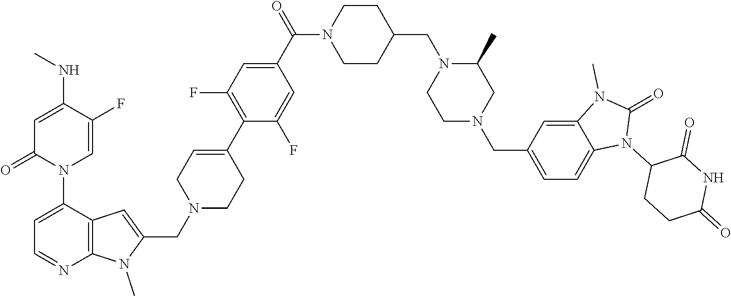 [SMILES: CNc1cc(=O)n(-c2ccnc3c2cc(CN2CC=C(c4c(F)cc(C(=O)N5CCC(CN6CCN(Cc7ccc8c(c7)n(C)c(=O)n8C7CCC(=O)NC7=O)C[C@@H]6C)CC5)cc4F)CC2)n3C)cc1F] |
| 3u55 and 3u56 | 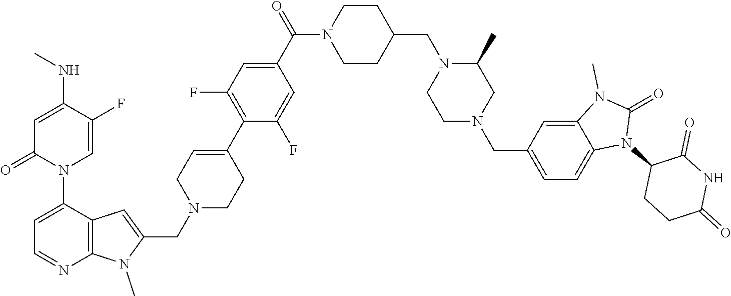 [SMILES: CNc1cc(=O)n(-c2ccnc3c2cc(CN2CC=C(c4c(F)cc(C(=O)N5CCC(CN6CCN(Cc7ccc8c(c7)n(C)c(=O)n8[C@@H]7CCC(=O)NC7=O)C[C@@H]6C)CC5)cc4F)CC2)n3C)cc1F] |
| | 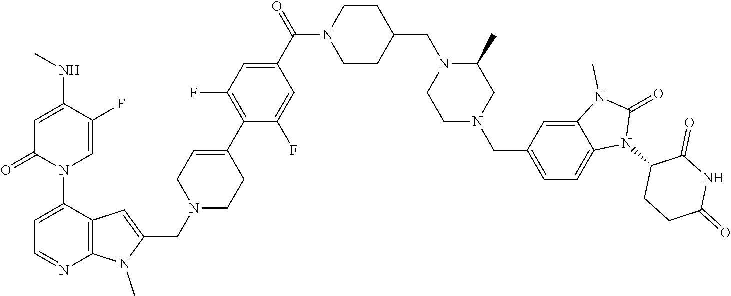 [SMILES: CNc1cc(=O)n(-c2ccnc3c2cc(CN2CC=C(c4c(F)cc(C(=O)N5CCC(CN6CCN(Cc7ccc8c(c7)n(C)c(=O)n8[C@H]7CCC(=O)NC7=O)C[C@@H]6C)CC5)cc4F)CC2)n3C)cc1F] |
| 3u57 | 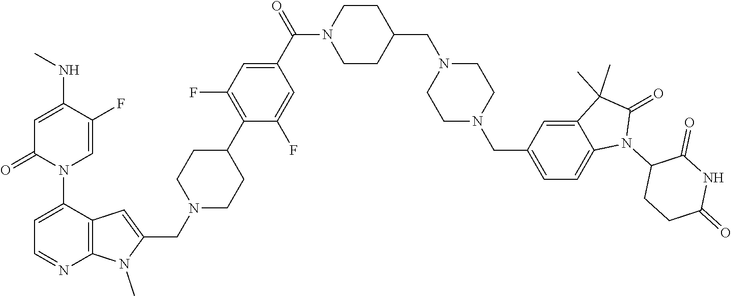 [SMILES: CNc1cc(=O)n(-c2ccnc3c2cc(CN2CCC(c4c(F)cc(C(=O)N5CCC(CN6CCN(Cc7ccc8c(c7)C(C)(C)C(=O)N8C7CCC(=O)NC7=O)CC6)CC5)cc4F)CC2)n3C)cc1F] |
| 3u58 and 3u59 | 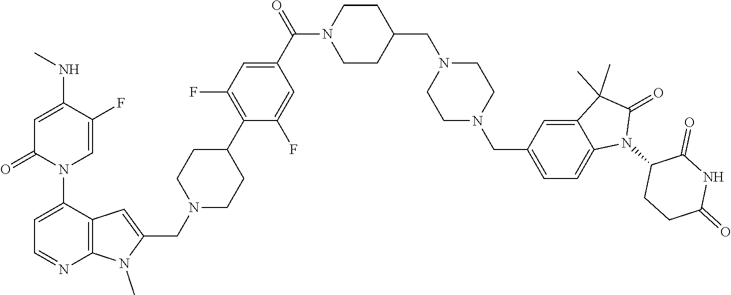 [SMILES: CNc1cc(=O)n(-c2ccnc3c2cc(CN2CCC(c4c(F)cc(C(=O)N5CCC(CN6CCN(Cc7ccc8c(c7)C(C)(C)C(=O)N8[C@H]7CCC(=O)NC7=O)CC6)CC5)cc4F)CC2)n3C)cc1F] |
| | 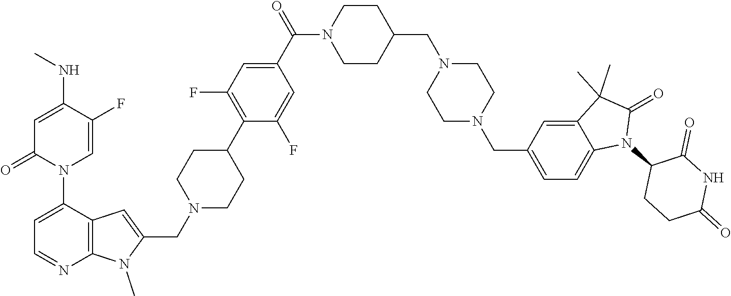 [SMILES: CNc1cc(=O)n(-c2ccnc3c2cc(CN2CCC(c4c(F)cc(C(=O)N5CCC(CN6CCN(Cc7ccc8c(c7)C(C)(C)C(=O)N8[C@@H]7CCC(=O)NC7=O)CC6)CC5)cc4F)CC2)n3C)cc1F] |
| 3u6 and 3u7 | 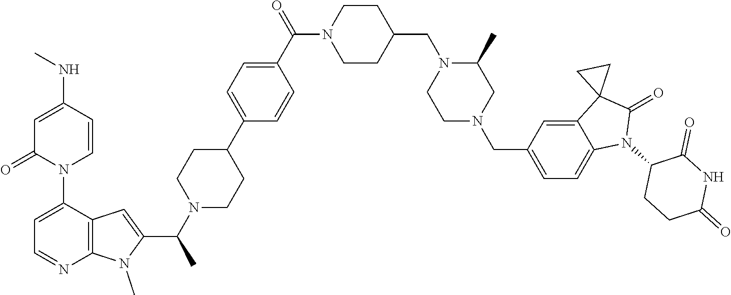 [SMILES: CNc1ccn(-c2ccnc3c2cc([C@H](C)N2CCC(c4ccc(C(=O)N5CCC(CN6CCN(Cc7ccc8c(c7)C7(CC7)C(=O)N8[C@H]7CCC(=O)NC7=O)C[C@@H]6C)CC5)cc4)CC2)n3C)c(=O)c1] |
| | 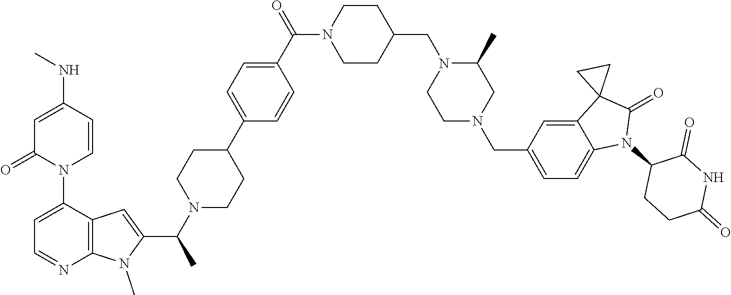 [SMILES: CNc1ccn(-c2ccnc3c2cc([C@H](C)N2CCC(c4ccc(C(=O)N5CCC(CN6CCN(Cc7ccc8c(c7)C7(CC7)C(=O)N8[C@@H]7CCC(=O)NC7=O)C[C@@H]6C)CC5)cc4)CC2)n3C)c(=O)c1] |
| 3u60 | 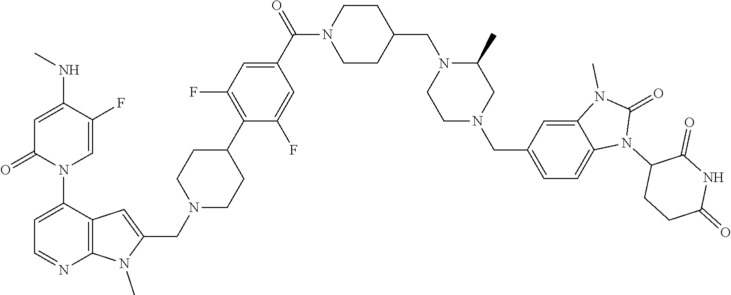 [SMILES: CNc1cc(=O)n(-c2ccnc3c2cc(CN2CCC(c4c(F)cc(C(=O)N5CCC(CN6CCN(Cc7ccc8c(c7)n(C)c(=O)n8C7CCC(=O)NC7=O)C[C@@H]6C)CC5)cc4F)CC2)n3C)cc1F] |
| 3u61 and 3u62 | 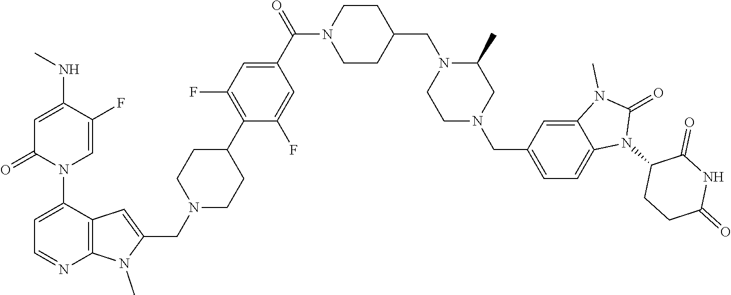 [SMILES: CNc1cc(=O)n(-c2ccnc3c2cc(CN2CCC(c4c(F)cc(C(=O)N5CCC(CN6CCN(Cc7ccc8c(c7)n(C)c(=O)n8[C@H]7CCC(=O)NC7=O)C[C@@H]6C)CC5)cc4F)CC2)n3C)cc1F] |
| | 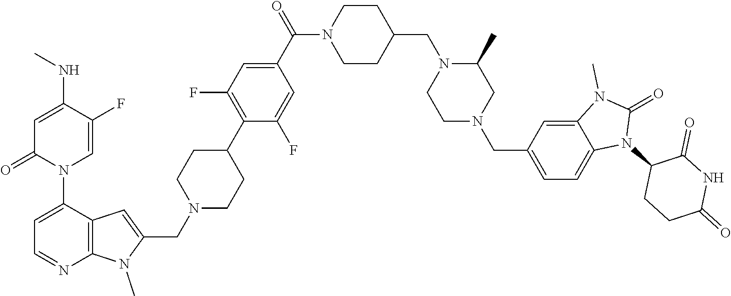 [SMILES: CNc1cc(=O)n(-c2ccnc3c2cc(CN2CCC(c4c(F)cc(C(=O)N5CCC(CN6CCN(Cc7ccc8c(c7)n(C)c(=O)n8[C@@H]7CCC(=O)NC7=O)C[C@@H]6C)CC5)cc4F)CC2)n3C)cc1F] |
| 3u63 | 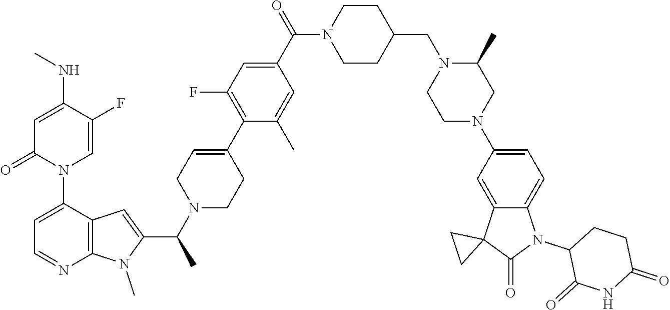 [SMILES: CNc1cc(=O)n(-c2ccnc3c2cc([C@H](C)N2CC=C(c4c(C)cc(C(=O)N5CCC(CN6CCN(c7ccc8c(c7)C7(CC7)C(=O)N8C7CCC(=O)NC7=O)C[C@@H]6C)CC5)cc4F)CC2)n3C)cc1F] |
| 3u64 and 3u65 | 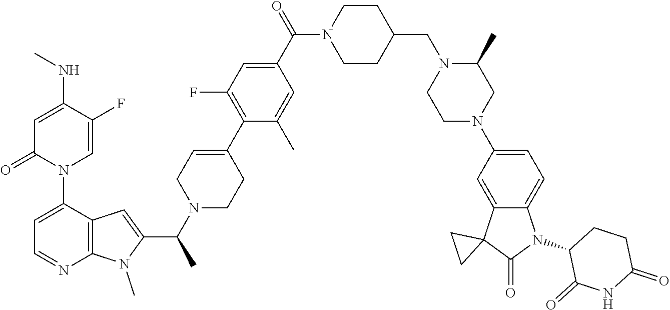 [SMILES: CNc1cc(=O)n(-c2ccnc3c2cc([C@H](C)N2CC=C(c4c(C)cc(C(=O)N5CCC(CN6CCN(c7ccc8c(c7)C7(CC7)C(=O)N8[C@@H]7CCC(=O)NC7=O)C[C@@H]6C)CC5)cc4F)CC2)n3C)cc1F] |
| | 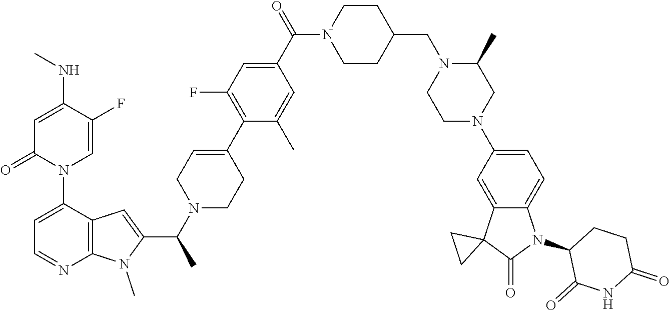 [SMILES: CNc1cc(=O)n(-c2ccnc3c2cc([C@H](C)N2CC=C(c4c(C)cc(C(=O)N5CCC(CN6CCN(c7ccc8c(c7)C7(CC7)C(=O)N8[C@H]7CCC(=O)NC7=O)C[C@@H]6C)CC5)cc4F)CC2)n3C)cc1F] |
| 3u66 | 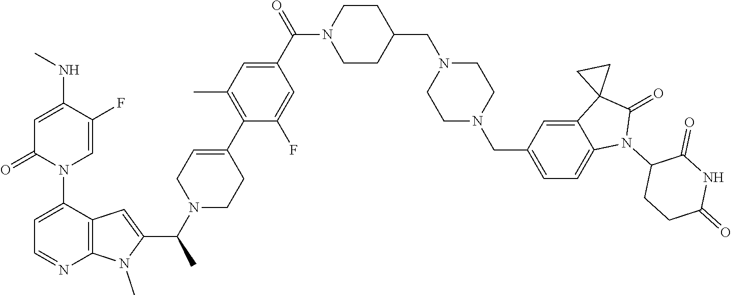 [SMILES: CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)CC6)CC5)cc4F)CC2)n3C)cc1F] |
| 3u67 and 3u68 | 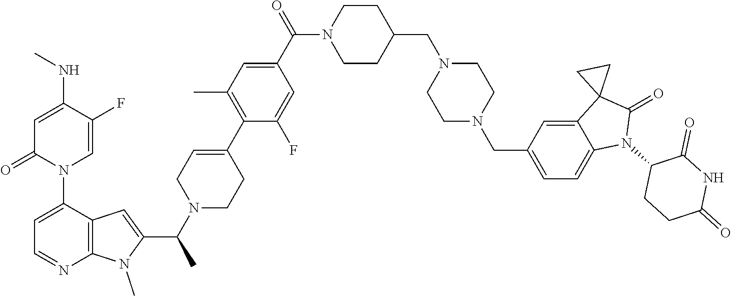 [SMILES: CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8[C@H]7CCC(=O)NC7=O)CC6)CC5)cc4F)CC2)n3C)cc1F] |
| | 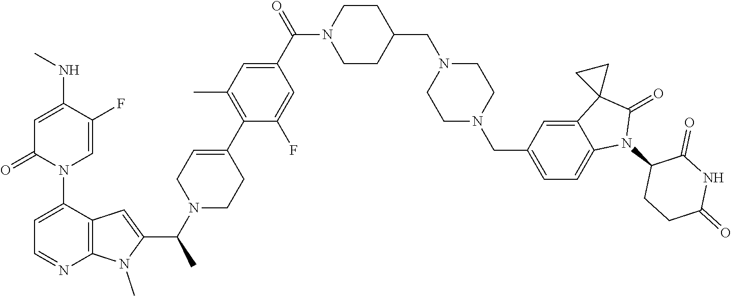 [SMILES: CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8[C@@H]7CCC(=O)NC7=O)CC6)CC5)cc4F)CC2)n3C)cc1F] |
| 3u69 | 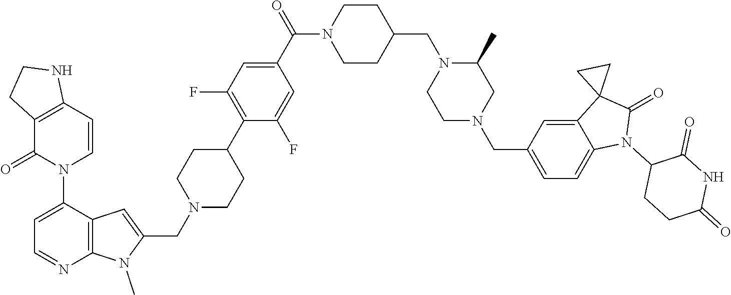 [SMILES: C[C@H]1CN(Cc2ccc3c(c2)C2(CC2)C(=O)N3C2CCC(=O)NC2=O)CCN1CC1CCN(C(=O)c2cc(F)c(C3CCN(Cc4cc5c(-n6ccc7c(c6=O)CCN7)ccnc5n4C)CC3)c(F)c2)CC1] |
| 3u70 | 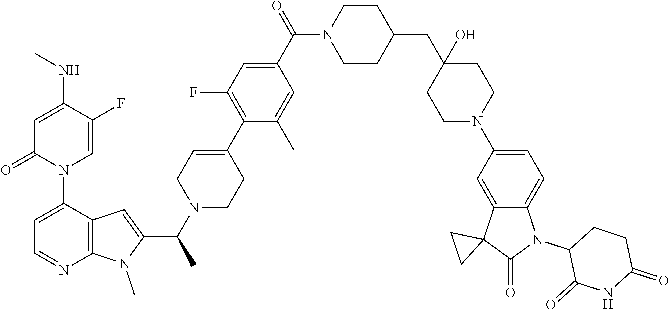 [SMILES: CNc1cc(=O)n(-c2ccnc3c2cc([C@H](C)N2CC=C(c4c(C)cc(C(=O)N5CCC(CC6(O)CCN(c7ccc8c(c7)C7(CC7)C(=O)N8C7CCC(=O)NC7=O)CC6)CC5)cc4F)CC2)n3C)cc1F] |
| 3u71 | 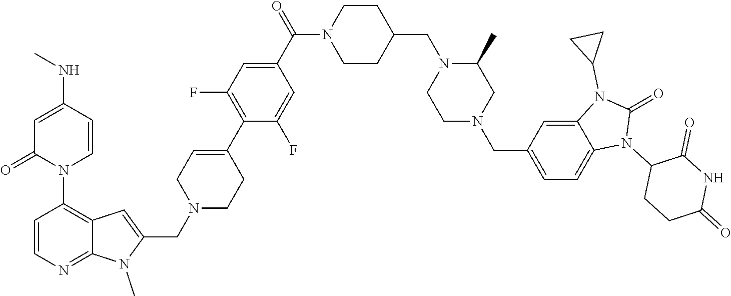 [SMILES: CNc1ccn(-c2ccnc3c2cc(CN2CC=C(c4c(F)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)c(=O)c1] |
| 3u72 and 3u73 | 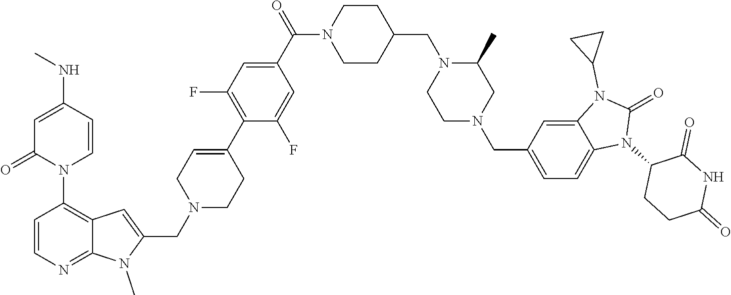 [SMILES: CNc1ccn(-c2ccnc3c2cc(CN2CC=C(c4c(F)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)c(=O)c1] |
| | 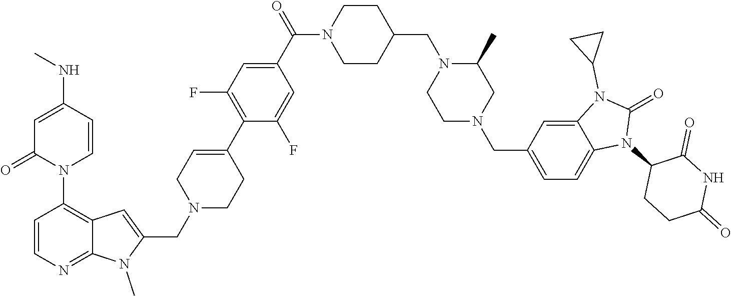 [SMILES: CNc1ccn(-c2ccnc3c2cc(CN2CC=C(c4c(F)cc(C(=O)N5CCC(CN6CCN(Cc7ccc8c(c7)n(C7CC7)c(=O)n8[C@@H]7CCC(=O)NC7=O)C[C@@H]6C)CC5)cc4F)CC2)n3C)c(=O)c1] |
| 3u74 | 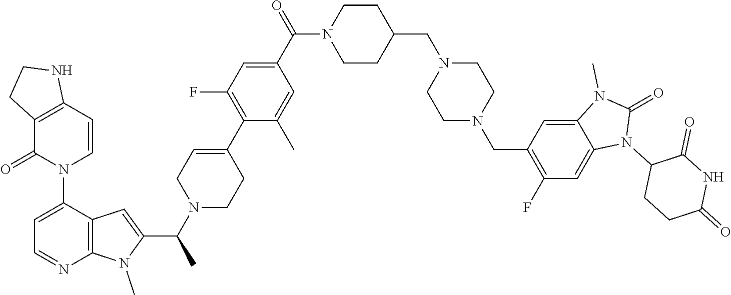 [SMILES: Cc1cc(C(=O)N2CCC(CN3CCN(Cc4cc5c(cc4F)n(C4CCC(=O)NC4=O)c(=O)n5C)CC3)CC2)cc(F)c1C1=CCN([C@@H](C)c2cc3c(-n4ccc5c(c4=O)CCN5)ccnc3n2C)CC1] |
| 3u75 and 3u76 | 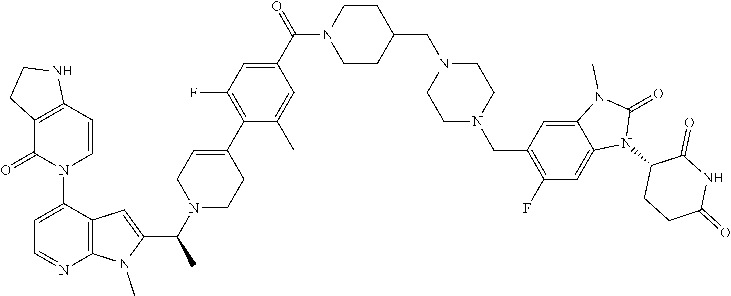 [SMILES: Cc1cc(C(=O)N2CCC(CN3CCN(Cc4cc5c(cc4F)n([C@H]4CCC(=O)NC4=O)c(=O)n5C)CC3)CC2)cc(F)c1C1=CCN([C@@H](C)c2cc3c(-n4ccc5c(c4=O)CCN5)ccnc3n2C)CC1] |
| | 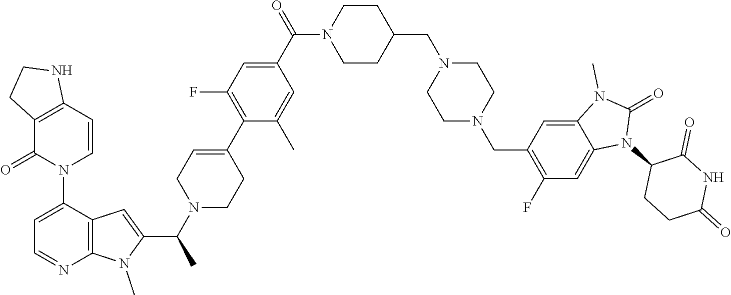 [SMILES: Cc1cc(C(=O)N2CCC(CN3CCN(Cc4cc5c(cc4F)n([C@@H]4CCC(=O)NC4=O)c(=O)n5C)CC3)CC2)cc(F)c1C1=CCN([C@@H](C)c2cc3c(-n4ccc5c(c4=O)CCN5)ccnc3n2C)CC1] |
| 3u77 | 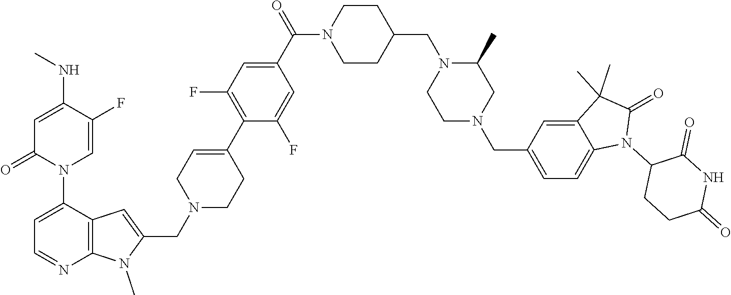 [SMILES: CNc1cc(=O)n(-c2ccnc3c2cc(CN2CC=C(c4c(F)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)cc1F] |
| 3u78 and 3u79 | 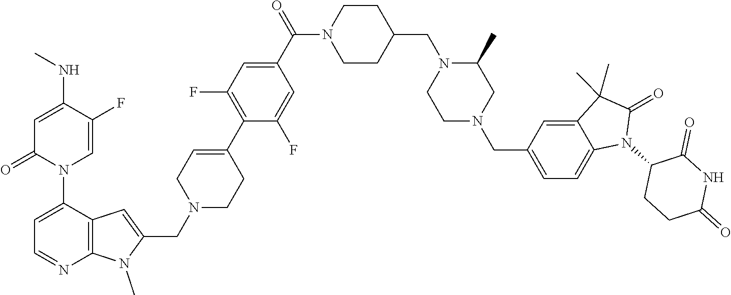 [SMILES: CNc1cc(=O)n(-c2ccnc3c2cc(CN2CC=C(c4c(F)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)cc1F] |
| | 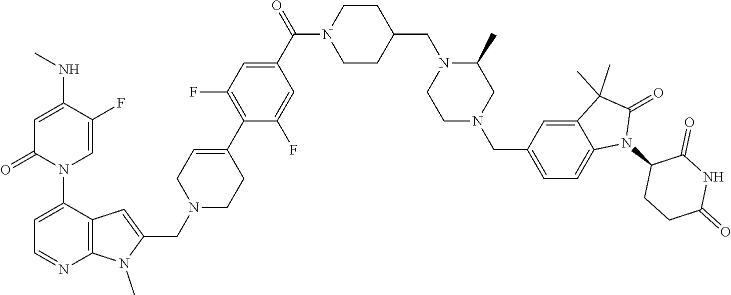 [SMILES: CNc1cc(=O)n(-c2ccnc3c2cc(CN2CC=C(c4c(F)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)cc1F] |
| 3u8 | 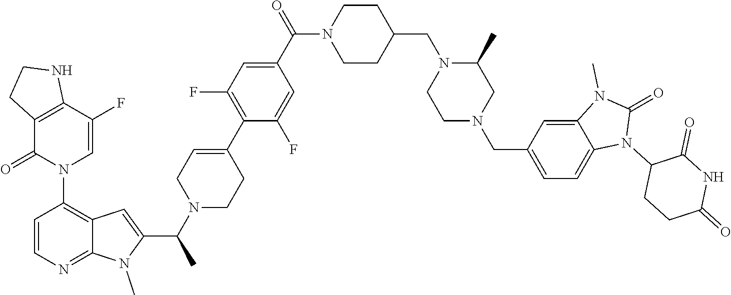 [SMILES: C[C@H]1CN(Cc2ccc3c(c2)n(C)c(=O)n3C2CCC(=O)NC2=O)CCN1CC1CCN(C(=O)c2cc(F)c(C3=CCN([C@@H](C)c4cc5c(-n6cc(F)c7c(c6=O)CCN7)ccnc5n4C)CC3)c(F)c2)CC1] |
| 3u80 | 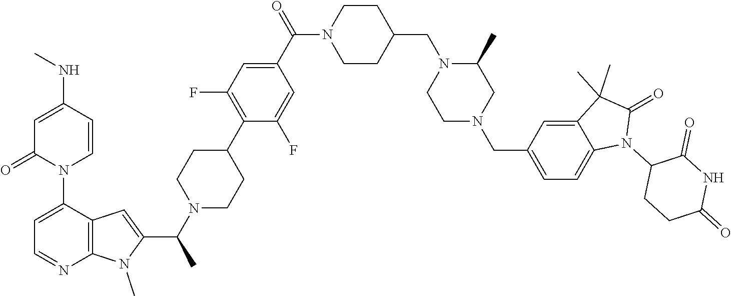 [SMILES: CNc1ccn(-c2ccnc3c2cc([C@H](C)N2CCC(c4c(F)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)c(=O)c1] |
| 3u81 and 3u82 | 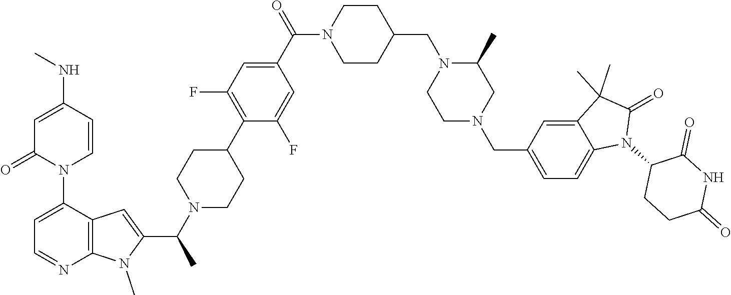 [SMILES: CNc1ccn(-c2ccnc3c2cc([C@H](C)N2CCC(c4c(F)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)c(=O)c1] |
| | 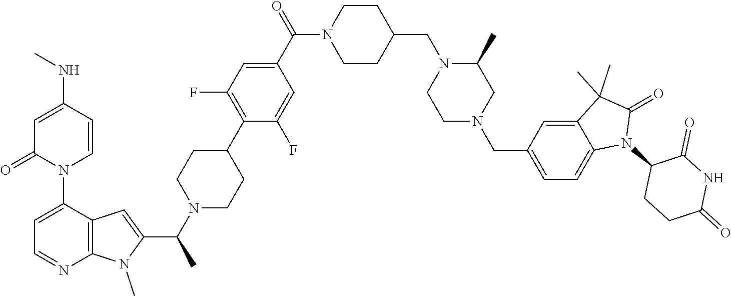 [SMILES: CNc1ccn(-c2ccnc3c2cc([C@H](C)N2CCC(c4c(F)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)c(=O)c1] |
| 3u83 | 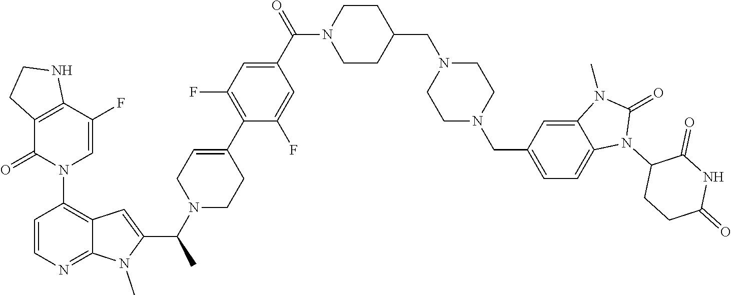 [SMILES: C[C@@H](c1cc2c(-n3cc(F)c4c(c3=O)CCN4)ccnc2n1C)N1CC=C(c2c(F)cc(C(=O)N3CCC(CN4CCN(Cc5ccc6c(c5)n(C)c(=O)n6C5CCC(=O)NC5=O)CC4)CC3)cc2F)CC1] |
| 3u84 | 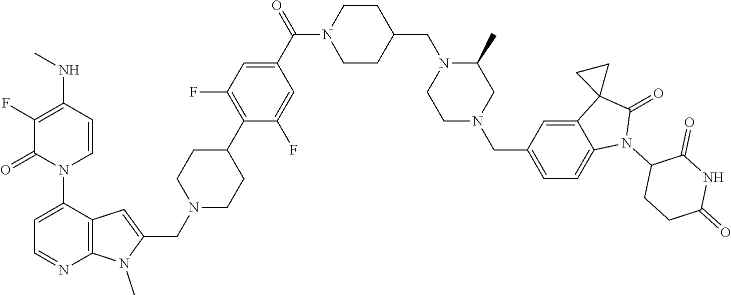 [SMILES: CNc1ccn(-c2ccnc3c2cc(CN2CCC(c4c(F)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)c(=O)c1F] |
| 3u85 | 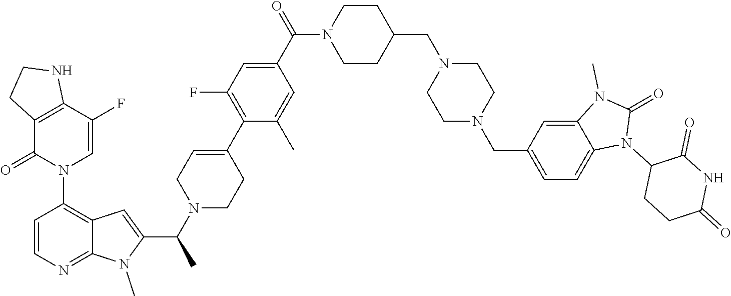 [SMILES: Cc1cc(C(=O)N2CCC(CN3CCN(Cc4ccc5c(c4)n(C)c(=O)n5C4CCC(=O)NC4=O)CC3)CC2)cc(F)c1C1=CCN([C@@H](C)c2cc3c(-n4cc(F)c5c(c4=O)CCN5)ccnc3n2C)CC1] |
| 3u86 | 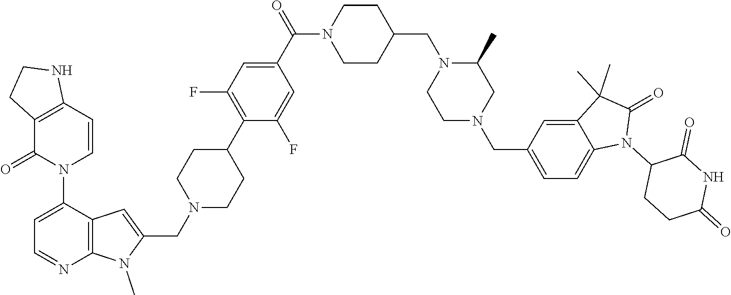 [SMILES: C[C@H]1CN(Cc2ccc3c(c2)C(C)(C)C(=O)N3C2CCC(=O)NC2=O)CCN1CC1CCN(C(=O)c2cc(F)c(C3CCN(Cc4cc5c(-n6ccc7c(c6=O)CCN7)ccnc5n4C)CC3)c(F)c2)CC1] |
| 3u87 | 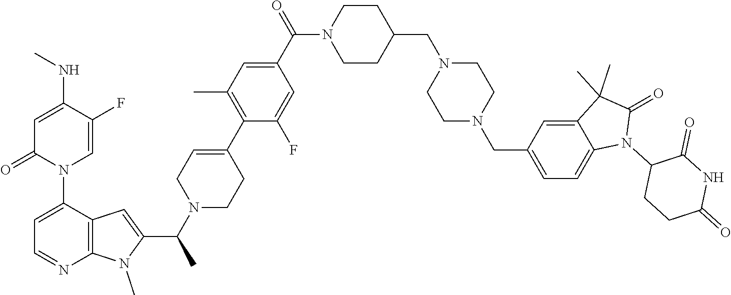 [SMILES: CNc1cc(=O)n(-c2ccnc3c2cc([C@H](C)N2CC=C(c4c(C)cc(C(=O)N5CCC(CN6CCN(Cc7ccc8c(c7)C(C)(C)C(=O)N8C7CCC(=O)NC7=O)CC6)CC5)cc4F)CC2)n3C)cc1F] |
| 3u88 and 3u89 | 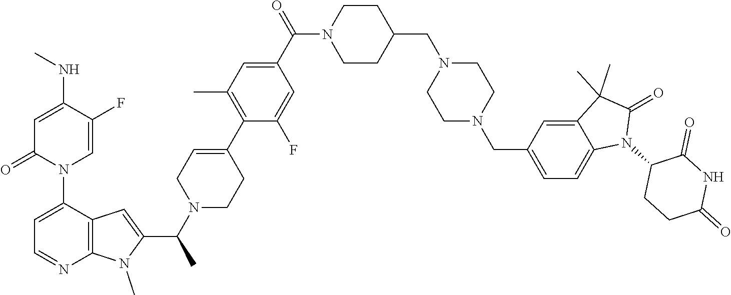 [SMILES: CNc1cc(=O)n(-c2ccnc3c2cc([C@H](C)N2CC=C(c4c(C)cc(C(=O)N5CCC(CN6CCN(Cc7ccc8c(c7)C(C)(C)C(=O)N8[C@H]7CCC(=O)NC7=O)CC6)CC5)cc4F)CC2)n3C)cc1F] |
| | 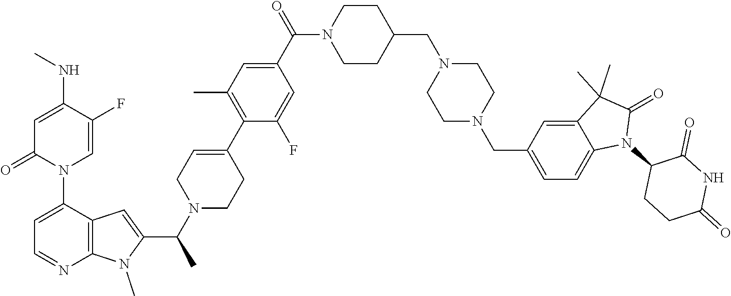 [SMILES: CNc1cc(=O)n(-c2ccnc3c2cc([C@H](C)N2CC=C(c4c(C)cc(C(=O)N5CCC(CN6CCN(Cc7ccc8c(c7)C(C)(C)C(=O)N8[C@@H]7CCC(=O)NC7=O)CC6)CC5)cc4F)CC2)n3C)cc1F] |
| 3u9 | 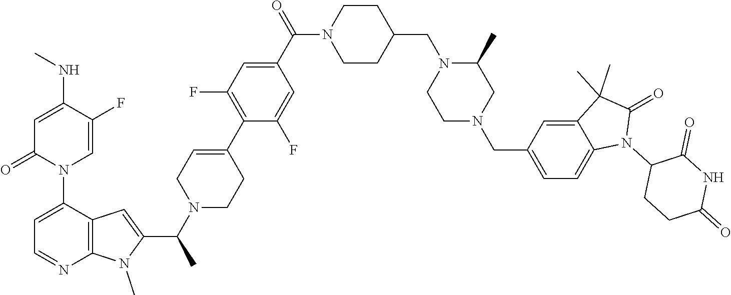 [SMILES: CNc1cc(=O)n(-c2ccnc3c2cc([C@H](C)N2CC=C(c4c(F)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)cc1F] |
| 3u90 | 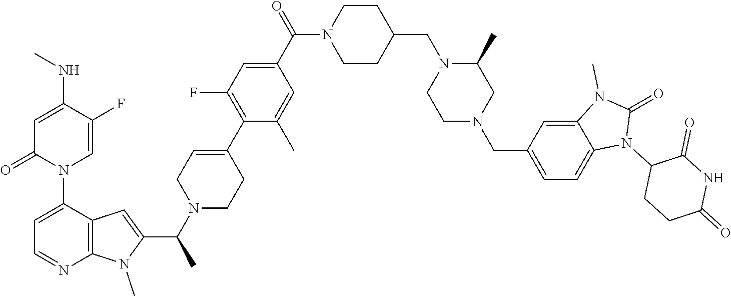 [SMILES: CNc1cc(=O)n(-c2ccnc3c2cc([C@H](C)N2CC=C(c4c(C)cc(C(=O)N5CCC(CN6CCN(Cc7ccc8c(c7)n(C)c(=O)n8C7CCC(=O)NC7=O)C[C@@H]6C)CC5)cc4F)CC2)n3C)cc1F] |
| 3u91 and 3u92 | 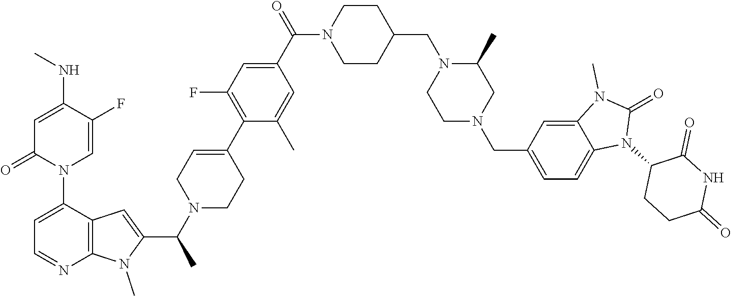 [SMILES: CNc1cc(=O)n(-c2ccnc3c2cc([C@H](C)N2CC=C(c4c(C)cc(C(=O)N5CCC(CN6CCN(Cc7ccc8c(c7)n(C)c(=O)n8[C@H]7CCC(=O)NC7=O)C[C@@H]6C)CC5)cc4F)CC2)n3C)cc1F] |
| | 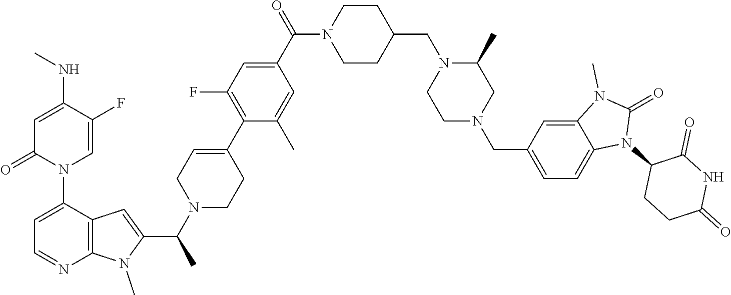 [SMILES: CNc1cc(=O)n(-c2ccnc3c2cc([C@H](C)N2CC=C(c4c(C)cc(C(=O)N5CCC(CN6CCN(Cc7ccc8c(c7)n(C)c(=O)n8[C@@H]7CCC(=O)NC7=O)C[C@@H]6C)CC5)cc4F)CC2)n3C)cc1F] |
| 3u93 | 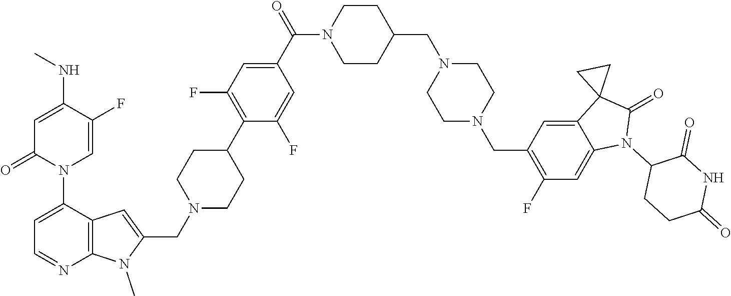 [SMILES: CNc1cc(=O)n(-c2ccnc3c2cc(CN2CCC(c4c(F)cc(C(=O)N5CCC(CN6CCN(Cc7cc8c(cc7F)N(C7CCC(=O)NC7=O)C(=O)C87CC7)CC6)CC5)cc4F)CC2)n3C)cc1F] |
| 3u94 and 3u95 | 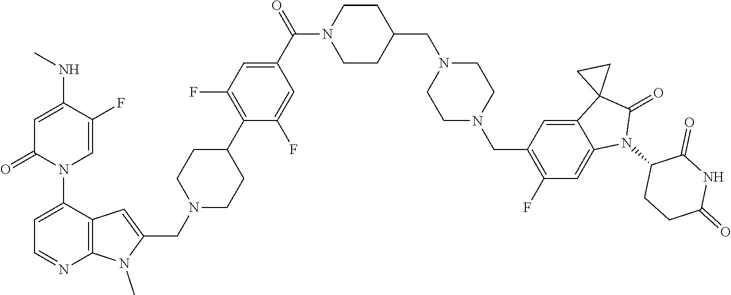 [SMILES: CNc1cc(=O)n(-c2ccnc3c2cc(CN2CCC(c4c(F)cc(C(=O)N5CCC(CN6CCN(Cc7cc8c(cc7F)N([C@H]7CCC(=O)NC7=O)C(=O)C87CC7)CC6)CC5)cc4F)CC2)n3C)cc1F] |
| | 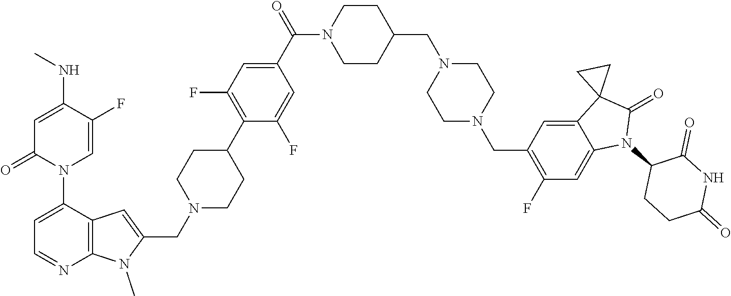 [SMILES: CNc1cc(=O)n(-c2ccnc3c2cc(CN2CCC(c4c(F)cc(C(=O)N5CCC(CN6CCN(Cc7cc8c(cc7F)N([C@@H]7CCC(=O)NC7=O)C(=O)C87CC7)CC6)CC5)cc4F)CC2)n3C)cc1F] |
| 3u96 | 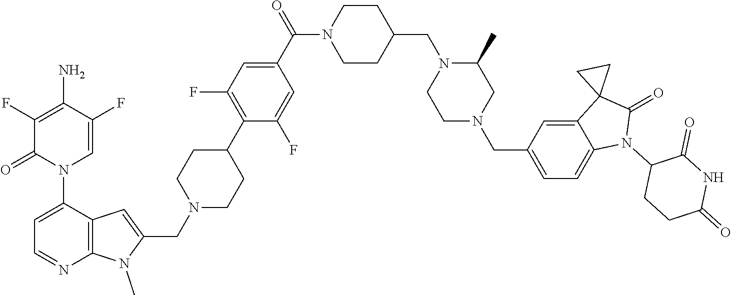 [SMILES: C[C@H]1CN(Cc2ccc3c(c2)C2(CC2)C(=O)N3C2CCC(=O)NC2=O)CCN1CC1CCN(C(=O)c2cc(F)c(C3CCN(Cc4cc5c(-n6cc(F)c(N)c(F)c6=O)ccnc5n4C)CC3)c(F)c2)CC1] |
| 3u97 and 3u98 | 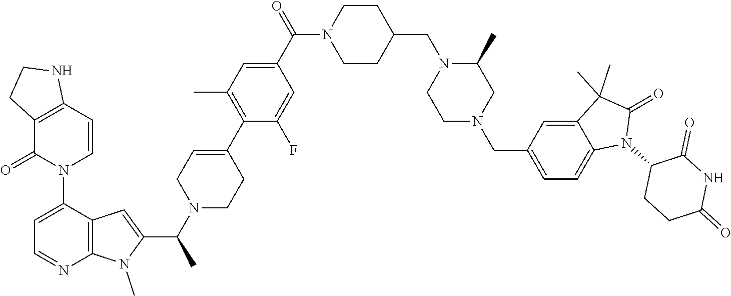 [SMILES: Cc1cc(C(=O)N2CCC(CN3CCN(Cc4ccc5c(c4)C(C)(C)C(=O)N5[C@H]4CCC(=O)NC4=O)C[C@@H]3C)CC2)cc(F)c1C1=CCN([C@@H](C)c2cc3c(-n4ccc5c(c4=O)CCN5)ccnc3n2C)CC1] |
| | 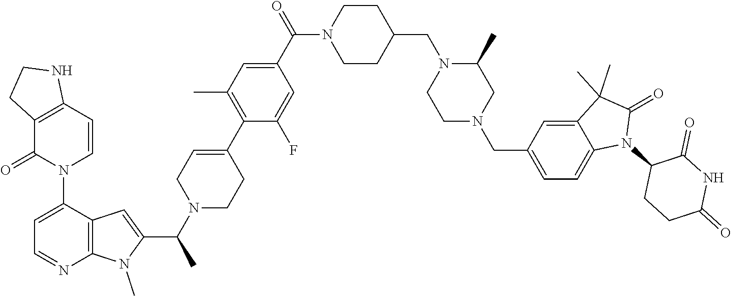 [SMILES: Cc1cc(C(=O)N2CCC(CN3CCN(Cc4ccc5c(c4)C(C)(C)C(=O)N5[C@@H]4CCC(=O)NC4=O)C[C@@H]3C)CC2)cc(F)c1C1=CCN([C@@H](C)c2cc3c(-n4ccc5c(c4=O)CCN5)ccnc3n2C)CC1] |
| 3h43 | 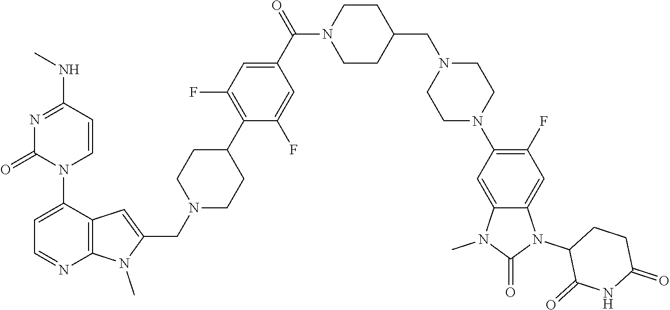 [SMILES: CNc1ccn(-c2ccnc3c2cc(CN2CCC(c4c(F)cc(C(=O)N5CCC(CN6CCN(c7cc8c(cc7F)n(C7CCC(=O)NC7=O)c(=O)n8C)CC6)CC5)cc4F)CC2)n3C)c(=O)n1] |

In one or more embodiments of the present disclosure, the compounds of formula (I) have an (EC₅₀) value in the Eotaxin-3 release assay of less than 100 micromolar, or of less than 10 micromolar, or less than 100 nanomolar, or less than 10 nanomolar.

In one or more embodiments of the present disclosure, the compounds of formula (I) have an (DC₅₀) value in the STAT6 assay describe below of less than 100 micromolar, or of less than 10 micromolar, or less than 100 nanomolar, or less than 10 nanomolar.

The compounds of formula (I) may be obtained in crystalline form either directly by concentration from an organic solvent or by crystallisation or recrystallisation from an organic solvent or mixture of said solvent and a co-solvent that may be organic or inorganic, such as water. The crystals may be isolated in essentially solvent-free form or as a solvate, such as a hydrate. The disclosure also provides crystalline forms of compounds of the disclosure, such as polymorphs and pseudopolymorphs, and also mixtures thereof.

Compounds of formula (I) may comprise asymmetrically substituted (chiral) carbon atoms which give rise to the existence of isomeric forms, e.g. enantiomers diastereomers, and other steroisomeric forms that can be defined, in terms of absolute stereochemistry, as (R)- or (S)-. The present disclosure provides all such isomers, either in optically pure form or as mixtures thereof (e.g. racemic mixtures or partially purified optical mixtures). Pure stereoisomeric forms of the compounds and the intermediates of this disclosure may be obtained by the application of procedures known in the art. The various isomeric forms may be separated by physical separation methods such as selective crystallization and chromatographic techniques, e.g. high pressure liquid chromatography using chiral stationary phases. Enantiomers may be separated from each other by selective crystallization of their diastereomeric salts which may be formed with optically active amines, or with optically active acids. Optically purified compounds may subsequently be liberated from said purified diastereomeric salts. Enantiomers may also be resolved by the formation of diastereomeric derivatives. Alternatively, enantiomers may be separated by chromatographic techniques using chiral stationary phases. Pure stereoisomeric forms may also be derived from the corresponding pure stereoisomeric forms of the appropriate starting materials, provided that the reaction occurs stereoselectively or stereospecifically. Preferably, if a specific stereoisomer is desired, said compound will be synthesized by stereoselective or stereospecific methods of preparation. These methods will advantageously employ chiral pure starting materials.

Furthermore, when a double bond or a fully or partially saturated ring system is present in the molecule geometric isomers may be formed. Any geometric isomer, as separated, pure or partially purified geometric isomers or mixtures thereof are included within the scope of the disclosure. When the compounds described herein contain olefinic double bonds or other centers of geometric asymmetry, and unless specified otherwise, it is intended that the compounds include both E and Z geometric isomers.

Likewise, all tautomeric forms are also intended to be included. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalemic mixture” is a mixture of stereoisomers at a ratio other than 1:1.

“Racemates” refers to a mixture of enantiomers. The mixture can comprise equal or unequal amounts of each enantiomer.

“STAT6 modulator” refers to compounds of the present disclosure that inhibit or reduce some or all of the activity of the Signal transducer and activator of transcription 6 (STAT6), including STAT6 degraders.

“STAT6 degrader” refers to compounds of the present disclosure that bind to and/or inhibit both STAT6 protein and an E3 ligase with measurable affinity, resulting in the ubiquitination and subsequent degradation of the STAT6 protein. In certain embodiments, a STAT6 degrader has an DC50 of less than about 50 pM, less than about 1 pM, less than about 500 nM, less than about 100 nM, less than about 10 nM, or less than about 1 nM. As used herein, the term “monovalent” refers to a degrader compound without an appended E3 ligase binding moiety.

STAT6-mediated disorders, diseases, and/or conditions refers to any disease or other deleterious condition in which STAT6 or a mutant thereof, are known to play a role.

A “subject” or “patient” is meant to describe a human or vertebrate animal, including a dog, cat, horse, cow, mouse, or the like.

“Treatment” or “treating” is an approach for obtaining beneficial or desired results including clinical results, such as inhibiting the disease or condition (e.g., decreasing one or more symptoms resulting from the disease or condition, and/or diminishing the extent of the disease or condition), slowing or arresting the development of one or more clinical symptoms associated with the disease or condition, and/or relieving the disease, enhancing the effect of another medication, delaying the progression of the disease, increasing quality of life, and/or prolonging survival.

In the compounds of formula (I), the atoms may exhibit their natural isotopic abundances, or one or more of the atoms may be artificially enriched in a particular isotope having the same atomic number, but an atomic mass or mass number different from the atomic mass or mass number found in nature. The present disclosure includes all suitable isotopic variations of the compounds of formula (I). For example, different isotopic forms of hydrogen include ¹H, ²H and ³H, different isotopic forms of carbon include ¹²C, ¹³C and ¹⁴C and different isotopic forms of nitrogen include ¹⁴N and ¹⁵N. Enriching for deuterium (²H) may for example increase in-vivo half-life or reduce dosage regimens, or may provide a compound useful as a standard for characterization of biological samples. Isotopically enriched compounds within formula (I) can be prepared by conventional techniques well known to a person skilled in the art or by processes analogous to those described in the procedures and examples herein using appropriate isotopically enriched reagents and/or intermediates.

In some embodiments, compounds described herein, or pharmaceutically acceptable salts, isomers, or a mixture thereof, have from 1 to n hydrogen atoms attached to a carbon atom replaced by a deuterium atom or D, in which n is the number of hydrogen atoms in the molecule. As known in the art, the deuterium atom is a non-radioactive isotope of the hydrogen atom. Such compounds can increase resistance to metabolism, and thus can be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism,” TRENDS PHARMACOL. SC., 5(12):524-527 (1984). Such compounds are synthesized by means well known in the art, for example by employing starting materials in which one or more hydrogen atoms have been replaced by deuterium.

In some embodiments, a compound of the disclosure is a solvate or a hydrate.

In one embodiment the disclosure provides a compound as above for use in therapy.

In one embodiment the disclosure provides a compound as above for use in the treatment of a disease, disorder or condition, which disease, disorder or condition is responsive of modulation of STAT-6.

In one embodiment the disclosure provides a compound as above for use in the treatment of autoimmune diseases.

The compounds of the present disclosure may be useful for preventing, treating or ameliorating diseases characterized by Th2-mediated inflammation such as atopic dermatitis, asthma, chronic rhinosinusitis with nasal polyposis, urticaria, rhinitis, eosinophilic esophagitis, food allergy, diffuse cutaneous systemic sclerosis, alopecia areata and/or COPD (chronic obstructive pulmonary disease) and different cancers such as lymphomas, triple negative breast cancer and solid fibrous cancers either as a stand-alone treatment or in combination with other anticancer drugs such as check point inhibitors.

In an embodiment the disclosure provides the use of a compound of formula (I) as defined above, in the manufacture of a medicament for the prophylaxis, treatment or amelioration of any of the following diseases characterized by Th2-mediated inflammation such as atopic dermatitis, asthma, chronic rhinosinusitis with nasal polyposis, urticaria, rhinitis, eosinophilic esophagitis, food allergy, diffuse cutaneous systemic sclerosis, alopecia areata and/or COPD (chronic obstructive pulmonary disease) and different cancers such as lymphomas, triple negative breast cancer and solid fibrous cancers either as a stand-alone treatment or in combination with other anticancer drugs such as check point inhibitors.

In an embodiment the disclosure provides a method of preventing, treating or ameliorating diseases characterized by Th2-mediated inflammation such as atopic dermatitis, asthma, chronic rhinosinusitis with nasal polyposis, urticaria, rhinitis, eosinophilic esophagitis, food allergy, diffuse cutaneous systemic sclerosis, alopecia areata and/or COPD (chronic obstructive pulmonary disease) and different cancers such as lymphomas, triple negative breast cancer and solid fibrous cancers either as a stand-alone treatment or in combination with other anticancer drugs such as check point inhibitors, the method comprising administering to a person suffering from at least one of said diseases an effective amount of one or more compounds according to formula (I), optionally together with a pharmaceutically acceptable carrier or one or more excipients, optionally in combination with other therapeutically active compounds.

In an embodiment the disclosure provides a method of preventing, treating or ameliorating autoimmune diseases, conditions characterized by Th2-mediated inflammation such as atopic dermatitis, asthma, chronic rhinosinusitis with nasal polyposis, urticaria, rhinitis, eosinophilic esophagitis, food allergy, diffuse cutaneous systemic sclerosis, alopecia areata and/or COPD (chronic obstructive pulmonary disease) and different cancers such as lymphomas, triple negative breast cancer and solid fibrous cancers either as a stand-alone treatment or in combination with other anticancer drugs such as check point inhibitors the method comprising administering to a person suffering from at least one of said diseases an effective amount of one or more compounds according to formula (I), optionally together with a pharmaceutically acceptable carrier or one or more excipients, optionally in combination with other therapeutically active compounds.

Besides being useful for human treatment, the compounds of the present disclosure may also be useful for veterinary treatment of animals including mammals such as horses, cattle, sheep, pigs, dogs, and cats.

### Pharmaceutical Compositions and Modes of Administration

For use in therapy, compounds of the present disclosure are typically in the form of a pharmaceutical composition. In some embodiments, the disclosure provides a pharmaceutical composition comprising a compound of Formula (I), optionally together with one or more other therapeutically active compound(s), together with a pharmaceutically acceptable excipient, vehicle or carrier(s). In some embodiments, the excipient is “acceptable” in the sense of being compatible with the other ingredients of the composition and not deleterious to the recipient thereof.

In some embodiments, the compound of the disclosure is provided in an amount of 0.0001-99.9% by weight of a formulation.

In the form of a dosage unit, a compound of the disclosure may be administered one or more times a day at appropriate intervals. In some embodiments, a dosage unit of a formulation contain between 0.001 mg and 1000 mg, such as between 0.01 mg and 300 mg of a compound of Formula (I).

A suitable dosage of the compound of the disclosure may depend, inter alia, on the age and condition of the patient, the severity of the disease to be treated and other factors well known to the practising physician. The compound may be administered either orally, parenterally, topically, transdermally or intradermally and other routes according to different dosing schedules, e.g. daily, weekly or with monthly intervals. In some embodiments, a single dose comprising a compound of the disclosure may provide an amount of the compound in the range from 0.001 to 400 mg/kg body weight.

In some embodiment, a compound of the disclosure is provided in combination with one or more therapeutically active compounds. If the treatment involves administration of another therapeutically active compound Goodman & Gilman's The Pharmacological Basis of Therapeutics, 9^{th} Ed., J. G. Hardman and L. E. Limbird (Eds.), McGraw-Hill 1995, may be consulted for useful dosages of said compounds.

The administration of a compound of the present disclosure with one or more other active compounds may be either concomitantly or sequentially.

The formulations include e.g. those in a form suitable for oral, rectal, parenteral transdermal, intradermal, ophthalmic, topical, nasal, sublingual or buccal administration.

The formulations may conveniently be presented in dosage unit form and may be prepared by but not restricted to any of the methods well known in the art of pharmacy, e.g. as disclosed in Remington, The Science and Practice of Pharmacy, 21ed ed., 2005. All methods include the step of bringing the active ingredient into association with the carrier, which constitutes one or more accessory ingredients. In general, the formulations may be prepared by uniformly and intimately bringing the active ingredient into association with a liquid carrier, semisolid carrier or a finely divided solid carrier or combinations of these, and then, if necessary, shaping the product into the desired formulation.

Formulations of the present disclosure suitable for oral and buccal administration may be in the form of discrete units as capsules, sachets, tablets, chewing gum or lozenges, each containing a predetermined amount of the active ingredient.

A tablet may be made by compressing, moulding or freeze drying the active ingredient optionally with one or more accessory ingredients. Compressed tablets may be prepared by compressing, in a suitable machine, the active ingredient(s) in a free-flowing form; for example with a lubricant; a disintegrating agent or a dispersing agent. Moulded tablets may be made by moulding, in a suitable machine, a mixture of the powdered active ingredient and suitable carrier. Freeze dried tablets may be formed in a freeze-dryer from a solution of the drug substance.

Formulations suitable for parenteral administration conveniently comprise a sterile oily or aqueous preparation of the active ingredients, which is preferably isotonic with the blood of the recipient, e.g. isotonic saline, isotonic glucose solution or buffer solution. Liposomal formulations are also suitable for parenteral administration.

Transdermal formulations may be in the form of a plaster, patch, microneedles, liposomal or nanoparticulate delivery systems or other cutaneous formulations applied to the skin.

Formulations suitable for ophthalmic administration may be in the form of a sterile aqueous preparation of the active ingredients. Liposomal formulations or biodegradable polymer systems may also be used to present the active ingredient for ophthalmic administration.

Formulations suitable for topical, such as dermal, intradermal or ophthalmic administration include liquid or semi-solid preparations, solutions or suspensions.

Formulations suitable for nasal or buccal administration include powder, self-propelling and spray formulations, such as aerosols and atomisers.

In some embodiments, administration can be topical (including transdermal, epidermal, ophthalmic and to mucous membranes including intranasal, vaginal and rectal delivery).

In some embodiments, pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may be necessary or desirable.

In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient.

In some embodiments, one or more compounds as disclosed herein or a pharmaceutical composition thereof is formulated for topical administration to the skin or mucosa (e.g., dermally or transdermally).

In some embodiments, topical compositions can include ointments and creams. In some embodiments, ointments are semisolid preparations that are typically based on petrolatum or other petroleum derivatives. In some embodiments, creams containing the selected active agent are typically viscous liquid or semisolid emulsions, often either oil-in-water or water-in-oil. For example, cream bases are typically water-washable, and contain an oil phase, an emulsifier and an aqueous phase. For example, the oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. In some embodiments, the emulsifier in a cream formulation is generally a nonionic, anionic, cationic or amphoteric surfactant. In some embodiments, as with other carriers or vehicles, an ointment base should be inert, stable, nonirritating and non-sensitizing.

All references, including publications, patent applications and patents, cited herein are hereby incorporated by reference in their entirety and to the same extent as if each reference were individually and specifically indicated to be incorporated by reference, regardless of any separately provided incorporation of particular documents made elsewhere herein.

### Combination Therapies

In one embodiment, the compounds disclosed herein may be used in combination with one or more additional therapeutic agents. In some embodiments, a compound of the present disclosure, or a pharmaceutically acceptable salt or stereoisomer thereof, can be combined with the therapeutically effective amount of one or more (e.g., one, two, three, four, one or two, one to three, or one to four) additional therapeutic agents.

In some embodiments, a compound of the disclosure, or a pharmaceutically acceptable salt or stereoisomer thereof, is co-administered with one or more agents useful for the treatment and/or prophylaxis of an inflammatory, and/or dermatologic disease or disorder, such as atopic dermatitis (AD). Non-limiting examples of such agents include topical corticosteroids (TCS) (e.g., desonid, hydrocortisone, fluocinolone, triamcinolone, betamethasone diproprionate), topical calcineurin inhibitors (TCI) (e.g., tacrolimus, pimecrolimus), cyclosporine, methotrexate, mycophenolate mofetil, azathioprine, interferon gamma, phosphodiesterase 4 (PDE4) inhibitor such as crisaborole, JAK inhibitor (e.g., ruxolitinib, upadacitinib, abrocitinib, baricitinib), dupilumab, and anti-IL-13 antibody (e.g., tralokinumab).

In some embodiments, a compound of the disclosure, or a pharmaceutically acceptable salt or stereoisomer thereof, is co-administered with one or more agents useful for the treatment and/or prophylaxis of a dermatologic condition, such as acne. Non-limiting examples of such agents include topical therapies such as benzoyl peroxide, topical retinoids, topical antibiotic, clascoterone, salicylic acid and azelaic acid; and systemic therapies such as doxycycline, minocycline, sarecycline, combined oral contraceptives, spironolactone, and isotretinoin.

In some embodiments, a compound of the disclosure, or a pharmaceutically acceptable salt or stereoisomer thereof, is co-administered with one or more agents useful for the treatment and/or prophylaxis of a dermatologic condition, such as alopecia areata. Non-limiting examples of such agents include topical therapies such as systemic corticosteroids (such as prednisolone), cyclosporine, azathioprine, methotrexate, sulfasalazine, simvastatin/exetimibe, inosiplex, antihistamines (such as fexofenadine), and oral JAK inhibitors (such as ritlecitinib or brepocitinib).

In some embodiments, a compound of the disclosure, or a pharmaceutically acceptable salt or stereoisomer thereof, is co-administered with one or more agents useful for the treatment and/or prophylaxis of a dermatologic condition, such as asthma. Non-limiting examples of such agents include inhaled ICS-formoterol (such as budesonide-formoterol), short-acting beta2 agonists (such as albuterol sulfate), leukotriene receptor antagonists (such as montelukast), immunoglobulin E antibodies (such as omalizumab) and long-acting muscarinic antagonists (such as tiotropium).

In some embodiments, a compound of the disclosure, or a pharmaceutically acceptable salt or stereoisomer thereof, is co-administered with one or more agents useful for the treatment and/or prophylaxis of a dermatologic condition, such as chronic obstructive pulmonary disease (COPD). Non-limiting examples of such agents include short-acting beta2 agonists (such as albuterol sulfate), short-acting muscarinic antagonists (such as aiprtropium), long-acting beta2 agonists (such as olodaterol), and long-acting muscarinic antagonists (such as tiotropium).

In some embodiments, a compound of the disclosure, or a pharmaceutically acceptable salt or stereoisomer thereof, is co-administered with one or more agents useful for the treatment and/or prophylaxis of a dermatologic condition, such as chronic rhinosinusitis with polyps. Non-limiting examples of such agents include intranasal corticosteroid, or biologics such as benralizumab (targets IL-5), dupilumab (targets IL-13), omalizumab (targets IgE).

In some embodiments, a compound of the disclosure, or a pharmaceutically acceptable salt or stereoisomer thereof, is co-administered with one or more agents useful for the treatment and/or prophylaxis of a dermatologic condition, such as contact dermatitis. Non-limiting examples of such agents include topical corticosteroids, topical calcineurin inhibitors (such as pimecrolimus, tacrolimus), topical phosphodiesterase 4 inhibitors, such as crisaborole, systemic immunosuppressants and modulators, such as systemic corticosteroids, methotrexate, azathioprine, mycophenolate mofetil, cyclosporine or dupilumab.

In some embodiments, a compound of the disclosure, or a pharmaceutically acceptable salt or stereoisomer thereof, is co-administered with one or more agents useful for the treatment and/or prophylaxis of a dermatologic condition, such as dermatomyositis. Non-limiting examples of such agents include topical corticosteroids, topical calcineurin inhibitors (such as pimecrolimus, tacrolimus), systemic corticosteroids (such as prednisone), antimalarials (such as hydroxychloroquine, cholorquine, quinacrine), methotrexate, mycophenolate mofetil, intravenous immunoglobulin, rituximab, and JAK inhibitors (such as tofacitinib).

In some embodiments, a compound of the disclosure, or a pharmaceutically acceptable salt or stereoisomer thereof, is co-administered with one or more agents useful for the treatment and/or prophylaxis of a dermatologic condition, such as esophageal eosinophilia. Non-limiting examples of such agents include systemic corticosteroids (such as budesonide, fluticasone, prednisone), topical corticosteroids, and proton pump inhibitors (such as omeprazole, esomeprazole, pantoprazole and lansoprazole).

In some embodiments, a compound of the disclosure, or a pharmaceutically acceptable salt or stereoisomer thereof, is co-administered with one or more agents useful for the treatment and/or prophylaxis of a dermatologic condition, such as psoriasis. Non-limiting examples of such agents include topical treatments such as topical corticosteroids (such as betamethasone dipropionate, clobetasol propionate, desoximetasone, diflorasone diacetate, fluocinonide, flurandrenolide, halobetasol propionate, amcinonide, mometasone furoate, triamcinolone acetonide, fluticasone propionate, hydrocortisone valerate, clocortolone pivalate), topical calcineurin inhibitors (such as pimecrolimus, tacrolimus), topical vitamin D analogues (such as calcipotriene, calcitriol, tacalcitol or mazacalcitol), topical retinoids (such as tazarotene); systemic nonbiologic therapies such as methotrexate, phosphodiesterase 4 inhibitors (such as apremilast), immunosuppressants (such as cyclosporine), oral retinoids (such as acitretin), oral Janus kinase inhibitors (such as tofacitinib), fumaric acid esters (such as dimethyl fumarate), systemic immunosuppressants and antimetabolites (such as hydroxyurea, mycophenolate mofetil, azathioprine, leflunomide, tacrolimus and thioguanine); and biologic therapies such as TNF-α inhibitors (such as etanercept, infliximab, adalimumab, certolizumab), IL-12/IL-23 inhibitors (such as ustekinumab), IL-17 inhibitors (such as secukinumab, ixekizumab, brodalumab), and IL-23 inhibitors (such as guselkumab, tildrakizumab, risankizumab).

In some embodiments, a compound of the disclosure, or a pharmaceutically acceptable salt or stereoisomer thereof, is co-administered with one or more agents useful for the treatment and/or prophylaxis of a dermatologic condition, such as scleroderma. Non-limiting examples of such agents include immunosuppressive treatments (such as methotrexate, mycophenolate mofetil, cyclophosphamide, tocilizumab, and rituximab), and autologous haematopoietic stem cell transplantation.

In some embodiments, a compound of the disclosure, or a pharmaceutically acceptable salt or stereoisomer thereof, is co-administered with one or more agents useful for the treatment and/or prophylaxis of a dermatologic condition, such as vitiligo. Non-limiting examples of such agents include topical treatments such as topical corticosteroids, topical calcineurin inhibitors (such as pimecrolimus, tacrolimus), topical vitamin D analogues (such as calcipotriene); and systemic therapies such as oral corticosteroids (such as betamethasone).

In some embodiments, an additional therapeutic agent includes one or more of 608, 610, 611, clindamycin phosphate+benzoyl peroxide, 101BHG-DO1, 1-H-11, 4P-022, 5-OXO-ETE receptor antagonists, 9MW-1911, AB-1000, AB-101a, abatacept, ABBV-712, ABCL-575, Ab-IPL-IL-17, ABM-125, abrocitinib, ABY-035/AFO2, ABY-062, AC-201, ACE-1334, acitretin, aclidinium bromide, aclidinium bromide+formoterol fumarate, acumapimod, AD-17002, adakitug, adalimumab, adapalene, adapalene+benzoyl peroxide, adapalene+clindamycin hydrochloride, adapalene+clindamycin phosphate, aderamastat, Adi, ADi-100, Adipocell, adipose tissue-derived mesenchymal stem cell-derived exosomes, adipose-derived stem cell therapy, AD-MSC-CM, ADSTEM, ADX-246, aerosolized hydroxychloroquine, afamelanotide, AJ-101, AJ-303, AK-101, AK-119, AKP-08, albuterol sulfate, ALD-R⁴⁹¹, alefacept, Allergovac depot, allogeneic adipose-derived mesenchymal stem cell therapy, allogeneic adult pluripotent stem cells, allogeneic mesenchymal stem cell therapy, allogeneic UC-MSC therapy, allogeneic umbilical cord mesenchymal stem cell therapy, AlloRx, alprazolam, AM-1476, ambroxol hydrochloride, AMG-0101, Amilo-5MER, aminolevulinic acid, aminolevulinic acid hydrochloride, aminopterin, amlitelimab, AMTX-100, AMTX-100 CF, Anapsos, ANB-032, ANB-101, anifrolumab, anti-CD19 CAR T cell therapy, anti-CD7 CAR T-cell therapy, anti-EMAP II fully humanized antibodies, anti-IL-4/IL-13 vaccine, anti-P2X7 monoclonal antibody humanized, anti-PAR2 therapeutics, antroquinonol, APD-588, APG-222, APG-777, APG-808, APG-990, APGT-001, APIRx-1603, apremilast, aprepitant, APT-101, AQ-001S, AQ-280, AR-100DP1, AR-110, arformoterol, ARG-201, ARGX-118, ARN-4079, ARO-MUC5AC, ARO-RAGE, ARO-TSLP, ARQ-234, arsenic trioxide, ARTS-011, AS-012, asengeprast, asivatrep, ASN-008, astegolimab, AT-004, AT-005, AT-0287, AT-193, ATB-1606, ATI-2138, ATL-105, ATR-006, ATR-01, ATTO-002, ATTO-1310, atuliflapon, AUR-101, auremolimab, autologous leukocyte cell therapy, avenciguat, AVI-3307, AVID-200, AVX-001, AWEPO-003, AX-158, AX-202, AZD-0284, AZD-0449, AZD-8630, azelaic acid, azelastine, azithromycin, B-244, Bacmune, bambuterol, baricitinib, barzolvolimab, BAT-6026, bazlitoran, BB-1511, BBACN, BBI-03, BBI-6000, BCG polysaccharide+nucleic acid, BCI-332, beclometasone dipropionate+formoterol fumarate, beclomethasone dipropionate, beclomethasone dipropionate+formoterol fumarate+glycopyrronium bromide, bedoradrine, begelomab, belimumab, belumosudil, bempikibart, bencycloquidium bromide, benralizumab, benzoyl peroxide, benzoyl peroxide+tretinoin, berdazimer sodium, bermekimab, bersiporocin dihydrochloride, bertilimumab, betamethasone, betamethasone dipropionate, betamethasone valerate, bexotegrast, BFP-002, BFP-102, BGB-23339, BI-1291583, BI-1323495, BI-765250, bilastine, bimekizumab, bimiralisib, BIO-11006 Inhalation Solution, BioLexa, BITT-CD4D11, BITT-CD4F10, BLR-200, BLU-808, BMS-986313, BMS-986322, BMS-986326, BMX-010, boningmycin, BOS-475, Bosakitug, bosentan, bovhyaluronidase azoximer, Box-5, BR-201, branebrutinib, BRE-AD01, brensocatib, brentuximab vedotin, brepocitinib, brilacidin, brilaroxazine hydrochloride, briquilimab, brodalumab, BSI-056T, BSI-502, BTX-1204, BTX-1308, BTX-1503, budesonide, budesonide+arformoterol, budesonide+formoterol, budesonide+formoterol fumarate, budesonide+procaterol hydrochloride, budesonide+salbutamol, budesonide+salmeterol, buloxibutid, BV-200 series, BVX-20, BZ-371, BZ-371B, C4X-6746, C-867, CABA-201, CAL-4, calcipotriol, calcipotriol+betamethasone, calcipotriol+betamethasone dipropionate, calcipotriol+cortisone, calcitriol, CALY-002, camoteskimab, CAN-10, cannabidiol, cannabidiol+dronabinol, cannabinoid CB2 receptor agonist antibody, carbon dioxide+perfluorooctyl bromide, cavosonstat, CB-06-01, CB2 receptor agonists, CB5138-3, CC-90006, CC-92252, CCI-15106, CCX-624, CD19-CAR-DNT, CEE-321, cendakimab, certolizumab pegol, CG-459, Chanllergen, CHF-6333, CHF-6366, CHF-6550, ciclesonide, ciclosporin, ciprofloxacin hydrochloride, CIT-013, CJRB-402, CKBA, clascoterone, CLBS-03, clindamycin, clindamycin phosphate+benzoyl peroxide, clindamycin phosphate+tretinoin, clobetasol propionate, clobetasol propionate+tretinoin, CM-101, CM-326, CMK-389, CMR-316, CMS-D001, ColiFin, COPD vaccine, cord blood derived stem cells, corticotropin, COYA-204, CPL-409116, crisaborole, CS-12192, CS-32582, CS-43001, CSJ-117, CSPCHA-115, CT-05, CT-303, CT-P55, CTX-101, CTXT-102, cudetaxestat sodium, CUR-N399, Cutaquig, CVXL-0074, CXF-11, CXG-86, CXG-87, cyproterone acetate+ethinyl estradiol, D-2570, D4-103-01, D4-103-02, D4-103-03, D4-103-04, daniluromer, dapansutrile, dapsone, daridorexant hydrochloride, daxdilimab, dazukibart, DB-007-4, DBI-001, DBM-1152A, DC-806, DC-853, deflazacort, delgocitinib, denifanstat, depemokimab, dersimelagon, desloratadine, desogestrel+ethinylestradiol, desonide, deucravacitinib, deuruxolitinib phosphate, dexamethasone sodium phosphate, dexpramipexole, difamilast, dimethyl fumarate, dimethyl fumarate+ethyl hydrogen fumarate calcium+ethyl hydrogen fumarate magnesium+ethyl hydrogen fumarate zinc, diroleuton, dithranol cream, divozilimab, DLQ-02, DLX-105, DLX-2323, DMT-210, DMT-310, DMX-700, DMXD-011, DNX-114, doxofylline, doxofylline (bronchiectasis), Alitair Pharmaceuticals, doxycycline hyclate, doxycycline hyclate (delayed release), Mayne, doxycycline hyclate (easy-to-swallow, acne, bacterial infection), Aqua Pharmaceuticals, DPT-0218, drospirenone+ethinylestradiol, dual alpha-V/beta-1 and alpha-5/beta-1 integrin inhibitors, dual AMCase/CHIT1 inhibitors, dual anti-CD19/anti-BAFF CAR T-cell therapy, dual JAK3/TEC inhibitor, dupilumab, dust mite vaccine, DW-2008S, DYV-024, DZ-2002, EB-005, EB-06, EBI-H, eblasakimab, efzofitimod, EI-001, elapegademase, elarekibep, emedastine, empasiprubart, ENA-002, ENB-109, endonuclease modulators, ENERGI-F708, enpatoran, ensifentrine, ensifentrine+glycopyrrolate, EP-104-GI, EP-262, epeleuton, Epi-13, epinastine hydrochloride, epinephrine, EpiTight, EPM-301, EQ-101, erdosteine, erlotinib, ESK-001, etanercept, EtanerRel, ETD-001, ETH-47, etrasimod, etrinabdione, EVX-B4, EYD-001, F-200, F-528, factor D inhibitor, farudodstat, FB-102, FB-401, FB-704A, FB-825, FB-918, FCR-001, FCX-013, fevipiprant, filgotinib maleate, fipaxalparant, flunisolide, fluocinonide, fluticasone, fluticasone+formoterol, fluticasone furoate, fluticasone furoate+umeclidinium+vilanterol, fluticasone furoate+vilanterol trifenatate, fluticasone propionate, fluticasone propionate+formoterol fumarate, fluticasone propionate+salbutamol sulfate, fluticasone propionate+salmeterol, fluticasone propionate+salmeterol xinafoate, formoterol, formoterol fumarate, formoterol fumarate+fluticasone propionate, formoterol fumarate+glycopyrronium bromide, FPP-003, FPP-005, froniglutide, FRTX-02, FTC-001, FWB-1313, FZ-007, FZJ-003, GABAA receptor agonists, Gamunex, GB-001, GB-0895, GD-134, GD-iExo-001, gefurulimab, GEN-501, GL-7190, GLPG-3667, glutathione+ascorbic acid+bicarbonate, glycopyrrolate+formoterol fumarate+budesonide, glycopyrronium+formoterol fumarate+fluticasone propionate, glycopyrronium+vilanterol, glycopyrronium bromide, glycopyrronium bromide+indacaterol maleate, GMDP, GM-XANTHO, GN-037, GNKS-356, GNR-068, GPCR antagonists, GR-010, GR-1501, GR-1802, GR-2002, GR-2301, Grastek, GRC-39815, GRT-6015, GSK-1070806, GSK-2831781, GSK-3862995B, GSK-3923868, GT-20029, gumokimab, gusacitinib, guselkumab, GZ-21T, H-018, halobetasol propionate, halobetasol propionate+tazarotene, halogenated xanthene, halometasone, HB00-17, HB-0034, HB-0043, HB-1734, HBM-9001, HBM-9378, HCW-9302, HDM-3010, HECB-1800301, HEMP-001, HI-1640V, histamine human immunoglobulin, Hizentra, HJ-787, HL-231, HLA-open conformer-specific monoclonal antibody, HLK-6002, house dust mite allergen, house dust mites immunotherapy, HP-1901, Hpb glutamate dehydrogenase modulator, HPP-737, HpVac-R¹³, HRG-2005, HRS-9821, HS-10374, HS-401, HT-004, HuL-001, human adipose-derived mesenchymal stem cells, human umbilical cord-derived mesenchymal stem cell therapy, HY-07170702, HY-072808, HY-1770, HY-209, hypericin, hypochlorous acid, HZ-J001, IBI-3002, IBI-356, IBIO-100, IBL-101, icanbelimod, ICP-332, ICP-488, iCP-NI, ifetroban, IFNalpha kinoide, IgE inhibitors, IHL-675A, IL-17 NanoAb, IL-25 targeted therapeutic, IL-4R alpha antagonist, IL-4Ra targeted therapeutic, ILB-2107, iloprost, IMB-101, IMG-007, IMG-008, IMG-036, immune globulin intravenous, imsidolimab, IMX-120, IN-A002, inaticabtagene autoleucel, INCB-054707, indacaterol, indacaterol acetate+glycopyrronium bromide+mometasone furoate, Indamet, inebilizumab, infliximab, Integrin alpha-2/beta-1 inhibitor, Integrin alpha-5/beta-1 inhibitor, Interleukin IL-17A inhibitor, INV-007, INV-103, INV-17, IPG-1094, IPG-7236, ipratropium+fenoterol, ipratropium bromide, ipratropium bromide+salbutamol sulfate, IR-444, IRL-201104, IRX-4204, isotretinoin, itepekimab, itolizumab, ivacaftor, ivarmacitinib sulfate, ivermectin, ixekizumab, izokibep, JadiCell therapy, JAK inhibitors, JAK-989, jaktinib dihydrochloride monohydrate, jaktinib hydrochloride, JK-0001, JK-0002, JNJ-1459, JNJ-2113, JNJ-3534, JNJ-67484703, JRF-106, JRF-401, JRP-878, JS-005, JTE-051, JTE-451, JW-1601, JW-202232, JW-2202, JYB-1904, JYP-0061, JYP-0066, K-1032, KB-5XX, KBL-693, KBL-697, KI-696, KINE-201, KITCL-27, KN-002, KP-470, KT-294, KT-474, KT-621, KX-826, KYV-101, L-608, LABA+LAMA therapy, Langopept, larsucosterol, LAS-200019, LBG-1600M, LCK inhibitor, lebrikizumab, lepzacitinib, levalbuterol, levalbuterol hydrochloride, levonorgestrel+ethinylestradiol, LG-283, LGM-1506, LGM-2605, LH-8, LIT-00505, lithium succinate, LMY-920, LNK-01001, LNK-01004, LNP-1955, LNR-653.1, londamocitinib, long acting beta agonist/long acting muscarinic agonist, long-acting aerosolized peptide-based therapy, lonodelestat acetate, lp-003, LP-0200, LQ-036, LQ-041, LQ-043, LR-19019, LR-20016, LT-002-158, lucinactant, lunsekimig, LUT-014, LW-104, LY-3509754, LY-3872386, LY-3972406, LYS-006, lysophospholipase inhibitor, LZM-012, M-119102, M3 muscarinic receptor antagonists, M-605110, M-610101, manfidokimab, masitinib, MAX-40070, maxacalcitol, maxacalcitol+betamethasone, MCM-001, MDI-1228, MDNA-413, MDPK-67b, ME-3183, Melgain, mepolizumab, mesalazine, Mesenchymal stem/stromal cell therapy, mesenchymoangioblast-derived mesenchymal stem cell therapy, metenkefalin acetate+tridecactide acetate, methotrexate, methyl aminolevulinate hydrochloride, methylprednisolone suleptanate, MG-01, MG-K10, MG-S-2525, MGY-1838, MG-ZG122, MH-004, MH-080, minocycline, minocycline+adapalene, minocycline hydrochloride, MIT-001, mitiperstat, Mitizax, MM-09, mometasone, mometasone+formoterol, mometasone furoate, mometasone furoate+indacaterol acetate, monlunabant, montelukast, montelukast sodium, montelukast sodium+levocetirizine dihydrochloride, mosedipimod, mouse monoclonal antibody against human interleukin-8, MP-1032, MSB-01, MSB-03, MSB-3163, MSM-605, MT-5562, MTC-896, mucosa-associated lymphoid tissue lymphoma translocation protein 1 inhibitors, mufemilast, MufroSyn, mugwort pollen allergen vaccine, muscarinic M3 receptor antagonist, MYJ-1633, nacystelyn, nadifloxacin, nadolol, nalfurafine, NBL-012, NCP-111, NCP-112, ND-003, NDX-3315, NDX-3324, nedocromil, nemolizumab, netakimab, nibrozetone, niclosamide, NIK inhibitors, nitric oxide, nitroglycerin, NLP-91, NM26-2198, nomacopan, norethindrone acetate+ethinylestradiol, noscapine/noscapine analogs, NP-339, nrf2 activator, NS-402, NTR-441, NVS-451, NX-73, OATD-01, OB-756, obefazimod, OC-701, OCR-4715, Octagam 10%, olodaterol, olodaterol hydrochloride+tiotropium bromide monohydrate 1, olopatadine, OLX-103, OM-001, omalizumab, omiganan pentahydrochloride, omilancor, OMN-71, ONO-4685, OP-2101, opinercept, OpSCF, ordesekimab, ORI-001, orismilast, ORKA-001, ORKA-002, orticumab, ozagrel hydrochloride, ozenoxacin, PA-9159, paridiprubart, PBF-680, PBI-100, PC-114, PDC-APB, PDE4 inhibitor, pegtarazimod, pemirolast, peresolimab, PF-07264660, PF-07275315, PG-011, PG-102, Viromed, phimelanotide, PHP-1212, PI3K-delta inhibitor, picankibart, piclidenoson, pimecrolimus, PIPE-791, pirfenidone, pitavastatin, PKC theta inhibitors, PLM-301, PNV-5032, POLB-002, ponesimod, potassium dobesilate, PR-023, pranlukast, pranlukast hydrate, PRCL-02, PrEP-001, prostaglandin D2 synthase inhibitors, Prozumab, PRP-PBMC autologous cellular therapy, PS-35, psoriasis therapeutics, PT-101, P-TET, PUL-042, PUR-0110, PUR-1800, PX-128, PX-130, PZ-07/2024, Q-1804, Q-301, QBKPN, QLM-3003, QN-02, QP-CO1, QRX-008, quisovalimab, QX-002-N, QX-004-N, QX-005-N, QX-007-N, QX-008-N, QX-009-N, QX-010-N, QY-101, QY-201, QY-211, R-187, R-552, rademikibart, rare phytocannabinoids, ravulizumab, RAY-121, RB-1000, RBM-009, RBN-012759, RBO-0987, RC-1416, RCD-405, recombinant midismase, reformulated calcipotriol+betamethasone, REGEND-001, REGN-1908-1909, remetinostat, remibrutinib, renzapride, repirinast, repurposed aldesleukin, Repurposed azeliragon, repurposed lenabasum, reslizumab, RESP-1000 series, RESP-2000 series, RESP-X, retinoic acid, revefenacin, REX-7117, rezpegaldesleukin, RG-6151, RG-6244, RG-6314, RG-6315, RG-6341, RG-6421, RGRN-305, rilzabrutinib, riociguat, risankizumab, ritlecitinib, rituximab, RLS-1496, RLV-102, rocatinlimab, roflumilast, ropsacitinib, ROR gamma T inverse agonists, ROR-gamma inverse agonists, rose bengal sodium, RP-3128, RSBT-001, RSS-0393, R-TPR-022, rupatadine+montelukast, RUTI, ruxolitinib, RYSW-01, SlPl agonist, salbutamol, salmeterol, salmeterol xinafoate+fluticasone propionate, SAMiRNA program, SAR-441566, SAR-443726, sarecycline, SB-010, SB-011, SCD-044, SCD-153, SCT-640A, SCT-650-C, SDC-1801, secukinumab, SEGRA, seletalisib, SEL-K2, selnoflast, seratrodast, SFA-002, SFA-004, SG-100, SGT-510, SH2 domain inhibitor program targeting STAT6, SHR-1703, SHR-1819, SHR-1905, SHR-4597, si-544, SIG-1322, SIG-1451, SIG-1456, SIM-0278, SIM-335, sitaxentan, SKI-O-703, SKL-XYZ, SLS-008, SM-17, small mobile stem cell therapy, SMET-D1, SNC-103, SNG-001, SNG-100, SNK-01, sodium chromoglycate, sodium pyruvate, sonelokimab, soquelitinib, sovleplenib, spesolimab, SPL84-23, SSGJ-621, SSS-07, ST-1830, stapokibart, STAR-0310, STAT3 inhibitor, STMC-103H, STS-01, STSA-1201, SUDO-286, SUL-238, SuperMApo, suplatast tosilate, SYHX-1901, SYX-5219, T-517, tacalcitol, tacrolimus, TAF-001, tagraxofusp, TAGX-0003, TAKC-02, tanimilast, TAP-1502, TAP-1503, tapinarof, Tavo-101, tazarotene, tazarotene+betamethasone dipropionate, tazarotene+clindamycin, TD-8236, TDM-180935, TDM-Atop01, TDM-Psor01, TDM-Scar01, telazorlimab, Tempol, temtokibart, teprotumumab, TER-101, terbutalin, terguride, tesnatilimab, TEV-48574, TEV-53275, tezepelumab, TFF-HMW-HA, Thalassophryne nattereri peptide, THB-001, theophylline, THOR-809, TI-520, TI-620, tibulizumab, tildrakizumab, timolumab, tiotropium, tiotropium bromide, tipelukast, tirbanibulin, TLL-018, TLY-012, TO-210, tofacitinib, tofacitinib+fingolimod, tofacitinib citrate, tonabacase, TOP-N44, TOP-N53, torudokimab, tosufloxacin, tozorakimab, TP-317, TQC-2731, TQC-2938, TQC-3564, TQC-3721, TQC-3927, TQH-2722, TQH-2929, TQH-3906, TQH-3910, trabikibart, tralokinumab, tranilast, transcription factor pathway inhibitor, tregalizumab, treprostinil, treprostinil diolamine, tretinoin, tretinoin+benzoyl peroxide, trifarotene, TRIV-509, TRN-157, TRPA1 antagonists, TS-0001, TT-01, TT-01688, tulinercept, tulobuterol, TVB-3567, UA-021, UB-221, UCB-1381, UCB-9741, ucenprubart, UHE-101, UHE-105, UI-009, UI-010, UI-031, UI-033, UI-034, ulobetasol, umbilical cord blood-derived stem cell therapy, UMC119-06, umeclidinium bromide, umeclidinium bromide+vilanterol trifenatate, upadacitinib, USP-4 inhibitors, ustekinumab, UTAA-09, VALERGEN-DS, vamorolone, vapendavir, vardenafil, VB-1953, VC-005, VDAA, VDAD, VDJ-006, VEGFR targeted DK4/10, venanprubart, VENT-03, verekitug, vilanterol+fluticasone furoate+glycopyrronium bromide, vipoglanstat hydrogensulfate, VISTA agonist, vixarelimab, VLRX-001, VM-AD, VNLG-152, vonifimod, voriconazole, votucalis, VRN-04, VS-105, VSG-158, VSTM-1 agonist, VT-014, VTH-212, vunakizumab, VYN-201, VYN-202, W16P-0576, WD-890, WM-1R3, WNT inhibitor, WNT pathway agonist antibodies, wnt pathway stimulator, WXFL-10203614, WXSH-0150, XCUR-17, XKH-001, XmAb-564, XT-0528, XZ.700, YH-25487, YH-35324, YKRH-00020, YR-001, Yso3, zabedosertib, zafirlukast, zasocitinib, ZB-168, Zemaira, ZeP-3, zetomipzomib, zevaquenabant, ZHB-107-108, zibotentan, zileuton, zirconium zr 89 crefmirlimab berdoxam, ZL-1102, ZPL-521, and/or bi-specific antibodies targeting one or more targets referenced herein.

In some embodiments a compound of the disclosure provided herein, or pharmaceutically acceptable salt or stereoisomer thereof, is administered with one or more therapeutic agents selected from a PPARd inhibitor, IRAK4 inhibitor, TPL2 inhibitor, α4β7 inhibitor, BTLA agonist, PD1 agonist, or an FXR agonist.

In some embodiments a compound of the disclosure provided herein, or pharmaceutically acceptable salt or stereoisomer thereof, is administered with one or more therapeutic agents selected from seladelpar, edecesertib, tilpisertib fosmecarbil, GS-1427, GS-0272, GS-0151, or cilofexor.

The benefit of combination may be increased efficacy and/or reduced side effects for a component as the dose of that component may be adjusted down to reduce its side effects while benefiting from its efficacy augmented by the efficacy of the compound of the present disclosure.

In some embodiments, the additional therapeutic agent includes an agent useful for modulating, treating, or preventing inflammation, such as a 4-1BB ligand, 5-Alpha-reductase inhibitor, 5-HT 1a receptor antagonist, 5-HT 1a receptor partial agonist, 5-HT 2a receptor antagonist, 5-HT 2a receptor partial agonist, 5-HT 2b receptor antagonist, 5-HT 3 receptor antagonist, 5-HT 4 receptor agonist, 5-HT 6 receptor antagonist, 5-HT 7 receptor antagonist, 5-Lipoxygenase activating protein inhibitor, 5-Lipoxygenase inhibitor, Accessory gene regulator A inhibitor, Acetaldehyde dehydrogenase modulator, Acetylcholine receptor agonist, Acetylcholine receptor antagonist, Acetylcholinesterase inhibitor, Acidic mammalian chitinase inhibitor, ACTH receptor agonist, Activity-dependent neuroprotector modulator, ADAM-33 inhibitor, ADAM-9 inhibitor, Adenosine A1 receptor antagonist, Adenosine A1 receptor modulator, Adenosine A2b receptor antagonist, Adenosine A3 receptor agonist, Adenosine A3 receptor antagonist, Adenosine deaminase stimulator, Adenosylhomocysteinase inhibitor, Adenylate cyclase stimulator, Adrenergic receptor agonist, Adrenocorticotrophic hormone ligand, Advanced glycosylation product receptor antagonist, AGER gene inhibitor, AIMP multisynthetase complex protein 1 inhibitor, Albumin antagonist, Alcohol dehydrogenase 5 inhibitor, Aldose reductase inhibitor, Alk-5 protein kinase inhibitor, Alpha 1 antitrypsin modulator, Alpha 1 antitrypsin stimulator, Alpha 1 proteinase inhibitor, Alpha 2 adrenoceptor agonist, Amiloride sensitive sodium channel inhibitor, AMP activated protein kinase alpha 2 stimulator, AMP activated protein kinase stimulator, Amyloid protein deposition inhibitor, Androgen receptor antagonist, Angiotensin II AT-1 receptor antagonist, Angiotensin II AT-2 receptor agonist, Anoctamin 1 stimulator, Aortic smooth muscle actin inhibitor, AP1 transcription factor modulator, Apelin receptor agonist, Apolipoprotein A antagonist, Apolipoprotein A5 stimulator, Apolipoprotein B modulator, Apolipoprotein E modulator, Apoptosis regulator Bel X inhibitor, Apoptosis regulator Bcl w inhibitor, APRIL receptor modulator, Aryl hydrocarbon receptor agonist, Aryl hydrocarbon receptor modulator, B and T lymphocyte attenuator stimulator, B-lymphocyte antigen CD19 inhibitor, B-lymphocyte antigen CD19 modulator, B-lymphocyte antigen CD20 inhibitor, B-lymphocyte stimulator ligand inhibitor, B-lymphocyte stimulator ligand modulator, Bcl-2 protein inhibitor, Beta 1 adrenoceptor antagonist, Beta 2 adrenoceptor agonist, Beta 2 adrenoceptor antagonist, Beta 2 adrenoceptor modulator, Beta adrenoceptor agonist, Beta amyloid antagonist, Beta-catenin inhibitor, Beta-catenin modulator, Bifunctional aminoacyl tRNA synthetase inhibitor, BMP10 gene inhibitor, BMP15 gene inhibitor, Bone marrow proteoglycan modulator, Botulinum toxin A stimulator, Bromodomain containing protein 1 inhibitor, Bromodomain containing protein inhibitor, Btk tyrosine kinase inhibitor, C-myc binding protein inhibitor, C-type lectin domain protein 4C inhibitor, Ca2+release activated Ca2+channel 1 inhibitor, Calcineurin inhibitor, Calcium channel inhibitor, Cannabinoid CB1 receptor antagonist, Cannabinoid CB1 receptor inverse agonist, Cannabinoid CB2 receptor agonist, Cannabinoid CB2 receptor modulator, Cannabinoid receptor agonist, Cannabinoid receptor antagonist, Cannabinoid receptor modulator, Catalase stimulator, CCL26 gene inhibitor, CCR3 chemokine modulator, CCR5 chemokine antagonist, CCR6 chemokine antagonist, CCR8 chemokine antagonist, CD1 lb antagonist, CD122 agonist, CD122 modulator, CD19 modulator, CD2 antagonist, CD223 modulator, CD3 modulator, CD30 modulator, CD4 antagonist, CD40 ligand receptor antagonist, CD47 antagonist, CDw123 modulator, Cell adhesion molecule inhibitor, Cell surface glycoprotein CD200R agonist, Cell surface glycoprotein MUC18 inhibitor, CFTR modulator, CFTR stimulator, Chaperonin stimulator, Chemokine receptor antagonist, Chitinase inhibitor, Collagen I agonist, Collagen I antagonist, Collagen modulator, Collagen VII antagonist, Complement C1q subcomponent inhibitor, Complement C1s subcomponent inhibitor, Complement C5 factor inhibitor, Complement factor C2 inhibitor, Complement factor D inhibitor, COVID19 spike glycoprotein modulator, CSF-1 antagonist, CXC10 chemokine ligand inhibitor, CXCR2 chemokine antagonist, cyclic GMP AMP synthase inhibitor, Cyclooxygenase inhibitor, Cytokine receptor agonist, Cytokine receptor antagonist, Cytokine receptor common beta chain inhibitor, Cytoplasmic protein NCK inhibitor, Cytosolic phospholipase A2 inhibitor, Cytotoxic T-lymphocyte protein-4 stimulator, Deoxyribonuclease gamma stimulator, DHFR inhibitor, Diacylglycerol O acyltransferase 1 inhibitor, Dihydroorotate dehydrogenase inhibitor, Dipeptidyl peptidase I inhibitor, Dipeptidyl peptidase IV inhibitor, DNA gyrase inhibitor, DNA methyltransferase inhibitor, Dopamine D2 receptor partial agonist, Dopamine D3 receptor agonist, Dopamine D3 receptor partial agonist, Dopamine D4 receptor partial agonist, DYRK-1 alpha protein kinase inhibitor, Ectonucleotide pyrophosphatase-PDE-2 inhibitor, EGF like module receptor 1 antagonist, EGFR family tyrosine kinase receptor inhibitor, Elastase inhibitor, Endonuclease modulator, Endostatin modulator, Endothelin ET-A receptor antagonist, Endothelin ET-B receptor antagonist, Enolase 1 inhibitor, Eosinophil peroxidase inhibitor, Eotaxin 2 ligand inhibitor, Eotaxin ligand inhibitor, EP4 prostanoid receptor antagonist, Epidermal growth factor receptor antagonist, Epidermal growth factor receptor modulator, Estradiol agonist, Estrogen receptor agonist, Extracellular signal related kinase-2 inhibitor, Facilitated glucose transporter-1 modulator, Fatty acid synthase inhibitor, FGF receptor antagonist, FGF-2 ligand, FGF-4 ligand, FGF3 receptor antagonist, Filaggrin stimulator, Flt3 tyrosine kinase inhibitor, FMLP related receptor I agonist, FMLP related receptor II agonist, Free fatty acid receptor 2 agonist, Free fatty acid receptor 3 agonist, Fyn tyrosine kinase inhibitor, FXR agonist, G-protein coupled bile acid receptor 1 agonist, G-protein coupled receptor-44 antagonist, G-protein coupled receptor-44 modulator, GABA A receptor agonist, GABA A receptor alpha-2 subunit modulator, GABA A receptor alpha-3 subunit modulator, GABA A receptor alpha-5 subunit modulator, Galectin-10 modulator, GATA 3 transcription factor inhibitor, Glucagon-like peptide 1 receptor agonist, Glucocorticoid receptor agonist, Glutamate dehydrogenase modulator, Glutamate receptor modulator, Glutathione dependent PGD synthase inhibitor, Glutathione independent PGD synthase inhibitor, Glutathione reductase inhibitor, GroEL protein 2 inhibitor, Guanylate cyclase stimulator, H+K+ATPase inhibitor, Heat shock protein inhibitor, Heme oxygenase 1 modulator, Heparin agonist, High mobility group protein B1 inhibitor, Histamine H1 receptor antagonist, Histamine H4 receptor antagonist, Histamine receptor antagonist, Histone deacetylase-1 inhibitor, Histone deacetylase-2 inhibitor, Histone deacetylase-2 stimulator, Histone deacetylase-3 inhibitor, Histone deacetylase-6 inhibitor, Histone H2A modulator, Histone H4 modulator, HMG CoA reductase inhibitor, Hsp 90 inhibitor, Hsp70 binding protein 1 inhibitor, Hyaluronidase stimulator, Hypoxia inducible factor stimulator, I-kappa B kinase beta inhibitor, I-kappa B kinase epsilon inhibitor, IgG receptor FcRn large subunit p51 modulator, IL-1 receptor accessory protein inhibitor, IL-1 receptor antagonist, IL-10 receptor agonist, IL-10 receptor antagonist, IL-12 receptor antagonist, IL-13 receptor antagonist, IL-13 receptor modulator, IL-15 receptor antagonist, IL-17 antagonist, IL-18 antagonist, IL-2 receptor alpha subunit stimulator, IL-2 receptor antagonist, IL-2 receptor modulator, IL-22 antagonist, IL-23 antagonist, IL-3 receptor modulator, IL-4 receptor antagonist, IL-4 receptor modulator, IL-5 receptor antagonist, IL-6 receptor antagonist, IL-7 receptor antagonist, IL-8 receptor antagonist, IL17RA gene inhibitor, IL2 gene stimulator, Immunoglobulin E antagonist, Immunoglobulin E modulator, Immunoglobulin G agonist, Immunoglobulin G1 modulator, Immunoglobulin agonist, Immunoglobulin kappa modulator, Immunoglobulin modulator, Inducible nitric oxide synthase inhibitor, Insulin receptor substrate-1 inhibitor, Insulin-like growth factor 1 receptor antagonist, Integrin alpha-2/beta-1 antagonist, Integrin alpha-4/beta-1 antagonist, Integrin alpha-4/beta-7 antagonist, Integrin alpha-5/beta-1 antagonist, Integrin alpha-5/beta-3 modulator, Integrin alpha-V/beta-1 antagonist, Integrin beta 1 binding protein modulator, Interferon alpha 14 ligand, Interferon alpha ligand inhibitor, Interferon beta ligand, Interferon beta ligand inhibitor, Interferon gamma ligand inhibitor, Interferon gamma receptor antagonist, Interferon type I receptor antagonist, Interleukin 1 delta ligand inhibitor, Interleukin 1 like receptor (IL33R) antagonist, Interleukin 1 like receptor 1 modulator, Interleukin 1 like receptor 2 inhibitor, Interleukin 13 ligand inhibitor, Interleukin 13 receptor alpha 1 antagonist, Interleukin 15 ligand inhibitor, Interleukin 17 ligand inhibitor, Interleukin 17A ligand inhibitor, Interleukin 17A ligand modulator, Interleukin 17E ligand inhibitor, Interleukin 17E ligand modulator, Interleukin 17F ligand inhibitor, Interleukin 17F ligand modulator, Interleukin 18 ligand inhibitor, Interleukin 23A inhibitor, Interleukin 31 ligand inhibitor, Interleukin 31 ligand modulator, Interleukin 33 ligand inhibitor, Interleukin 33 ligand modulator, Interleukin receptor 17A antagonist, Interleukin receptor 17B antagonist, Interleukin-1 alpha ligand inhibitor, Interleukin-1 beta ligand modulator, Interleukin-1 ligand inhibitor, Interleukin-2 ligand, Interleukin-2 ligand inhibitor, Interleukin-31 receptor modulator, Interleukin-4 ligand inhibitor, Interleukin-5 ligand inhibitor, Interleukin-6 ligand inhibitor, Interleukin-8 ligand inhibitor, Interleukin-9 ligand inhibitor, IRAK-4 protein kinase inhibitor, IRAK-4 protein kinase degrader, Itk tyrosine kinase inhibitor, JAK tyrosine kinase inhibitor, Jak1 tyrosine kinase inhibitor, Jak2 tyrosine kinase inhibitor, Jak3 tyrosine kinase inhibitor, Jun N terminal kinase inhibitor, Kallikrein 2 inhibitor, Kallikrein 5 modulator, Kallikrein 7 inhibitor, Kallikrein 7 modulator, Kallikrein inhibitor, KCNA voltage-gated potassium channel-3 inhibitor, Kelch like ECH associated protein 1 inhibitor, Kelch like ECH associated protein 1 modulator, Kit tyrosine kinase inhibitor, LanC like protein 2 stimulator, Lanosterol-14 demethylase inhibitor, Lek tyrosine kinase inhibitor, Lectin mannose binding protein inhibitor, Leukocyte Ig like receptor A4 modulator, Leukocyte elastase inhibitor, Leukotriene A4 hydrolase inhibitor, Leukotriene BLT receptor antagonist, Leukotriene C4 antagonist, Leukotriene C4 synthase inhibitor, Leukotriene D4 agonist, Leukotriene D4 antagonist, Leukotriene E4 antagonist, Leukotriene receptor antagonist, Liver X receptor agonist, LOXL2 gene inhibitor, Lung surfactant associated protein D stimulator, Lyn tyrosine kinase inhibitor, Lysophosphatidate-1 receptor antagonist, Lysophospholipase inhibitor, Macrophage migration inhibitory factor inhibitor, Major allergen I polypeptide chain 2 inhibitor, Major allergen inhibitor, MALT protein 1 inhibitor, Mannan-binding lectin serine protease inhibitor, MAP kinase modulator, MAPKAPK2 inhibitor, MARCKS protein inhibitor, Mas-related G-protein receptor X2 antagonist, Mas-related G-protein receptor X2 inhibitor, MEK protein kinase inhibitor, MEK-1 protein kinase inhibitor, Melanocortin MCl receptor agonist, Melanocortin MC5 receptor antagonist, Melanocortin receptor agonist, Melanocyte stimulating hormone ligand, Membrane copper amine oxidase inhibitor, Metalloprotease-12 inhibitor, MEX3B gene inhibitor, Mineralocorticoid receptor antagonist, MIP 3 alpha ligand inhibitor, Mite allergen modulator, Mitochondrial 10 kDa heat shock protein stimulator, MKL myocardin like protein inhibitor, MMP1 gene stimulator, MNK protein kinase inhibitor, Monocyte chemotactic protein 1 ligand inhibitor, MS4A2 gene modulator, mTOR complex 1 inhibitor, mTOR complex 2 inhibitor, MUC5AC gene inhibitor, Muscarinic M1 receptor antagonist, Muscarinic M2 receptor antagonist, Muscarinic M3 receptor antagonist, Muscarinic M4 receptor antagonist, Muscarinic M5 receptor antagonist, Muscarinic receptor agonist, Muscarinic receptor antagonist, Muscarinic receptor modulator, Myeloperoxidase inhibitor, Myosin heavy chain inhibitor, NACHT LRR PYD domain protein 3 inhibitor, NEDD4 family interacting protein 1 stimulator, Neuropilin 2 modulator, Neutral endopeptidase inhibitor, NFE2L2 gene stimulator, Nicotinic ACh receptor alpha 7 subunit stimulator, Nicotinic acetylcholine receptor agonist, NK1 receptor antagonist, NKG2 D activating NK receptor antagonist, NLR family member X1 stimulator, Non receptor tyrosine kinase TYK2 antagonist, Nuclear erythroid 2-related factor 1 stimulator, Nuclear erythroid 2-related factor 2 stimulator, Nuclear erythroid 2-related factor inhibitor, Nuclear factor kappa B gene inhibitor, Nuclear factor kappa B inducing kinase inhibitor, Nuclear factor kappa B inhibitor, Nuclear factor kappa B modulator, Oncostatin M receptor subunit beta inhibitor, Opioid growth factor receptor agonist, Opioid receptor delta antagonist, Opioid receptor kappa agonist, Orexin 1 receptor antagonist, Orexin 2 receptor antagonist, Orphan nuclear receptor antagonist, Outer membrane protein modulator, OX-40 receptor agonist, OX-40 receptor antagonist, OX40 ligand inhibitor, OX40 ligand modulator, Oxoeicosanoid receptor 1 antagonist, P-Glycoprotein inhibitor, P-selectin glycoprotein ligand-1 inhibitor, P2X2 purinoceptor antagonist, P2X3 purinoceptor antagonist, P2X7 purinoceptor modulator, P2Y6 purinoceptor modulator, p38 MAP kinase inhibitor, p53 tumor suppressor protein stimulator, PcrV protein type III inhibitor, PDE 3 inhibitor, PDE 4 inhibitor, PDE 4b inhibitor, PDE 4d inhibitor, PDE 5 inhibitor, PDGF receptor antagonist, Peptidase 1 inhibitor, PGD2 antagonist, PGI2 agonist, Phosphatidylinositol 4 kinase beta inhibitor, Phosphoinositide 3-kinase inhibitor, Phosphoinositide-3 kinase delta inhibitor, Phospholipase A2 inhibitor, Phospholipase C inhibitor, PIM-1 protein kinase inhibitor, PIM-2 protein kinase inhibitor, PIM-3 protein kinase inhibitor, Placenta growth factor ligand inhibitor, Plasminogen activator inhibitor 1 inhibitor, Platelet activating factor receptor antagonist, Poly ADP ribose polymerase 14 inhibitor, PPAR gamma agonist, PPAR gene modulator, Progesterone receptor agonist, Programmed cell death ligand 1 modulator, Programmed cell death protein 1 modulator, Programmed cell death protein 1 stimulator, Prostaglandin E synthase inhibitor, Prostaglandin E synthase-1 inhibitor, Protease inhibitor, Protease-activated receptor-2 antagonist, Proteasome beta-8 subunit modulator, Proteasome inhibitor, Protein kinase C theta inhibitor, Protein kinase inhibitor, Protein NOV homolog modulator, Protein tyrosine kinase inhibitor, Protoporphyrinogen oxidase modulator, Pyruvate kinase muscle isozyme stimulator, Raf B protein kinase inhibitor, Ras gene inhibitor, Reactive oxygen species modulator inhibitor, Ret tyrosine kinase receptor inhibitor, Retinoic acid receptor agonist, Retinoic acid receptor antagonist, Retinoic acid receptor gamma agonist, Retinoic acid receptor gamma antagonist, Retinoic acid receptor gamma inverse agonist, Retinoic acid receptor modulator, Retinoid receptor agonist, Retinoid X receptor agonist, Retinoid X receptor modulator, Retinoid Z receptor gamma antagonist, Retinoid Z receptor gamma inverse agonist, Rev protein modulator, Rho associated protein kinase 1 inhibitor, Rho associated protein kinase 2 inhibitor, Ribonuclease P inhibitor, Ribonuclease stimulator, RIP-1 kinase inhibitor, S100 calcium binding protein A4 inhibitor, S100A8 gene inhibitor, S100A9 gene inhibitor, SARS coronavirus 3C protease like inhibitor, Secretory phospholipase A2 receptor antagonist, Serine protease inhibitor, Serine threonine protein kinase TBK1 inhibitor, Serum amyloid A protein modulator, SH2 domain containing protein inhibitor, Sialic acid-binding Ig-like lectin 8 inhibitor, SIRT3 gene stimulator, SMAD inhibitor, SNAIl transcription factor inhibitor, SOD3 gene stimulator, Sodium channel inhibitor, Sphingosine 1 phosphate phosphatase 1 stimulator, Sphingosine-1-phosphate receptor-1 agonist, Sphingosine-1-phosphate receptor-1 antagonist, Sphingosine-1-phosphate receptor-1 modulator, Sphingosine-1-phosphate receptor-3 modulator, Sphingosine-1-phosphate receptor-4 antagonist, Sphingosine-1-phosphate receptor-4 modulator, Sphingosine-1-phosphate receptor-5 modulator, Sphingosylphosphorylcholine receptor antagonist, Src tyrosine kinase inhibitor, STAT inhibitor, STAT-1 modulator, STAT-3 inhibitor, STAT-5 inhibitor, STAT-6 inhibitor, STAT-6 degrader, Stearoyl CoA desaturase-1 inhibitor, Stress induced secreted protein 1 stimulator, Superoxide dismutase modulator, Superoxide dismutase stimulator, Syk tyrosine kinase inhibitor, Synuclein alpha inhibitor, T cell receptor antagonist, T cell receptor modulator, T cell surface glycoprotein CD28 inhibitor, T-cell antigen CD7 modulator, T-cell differentiation antigen CD6 inhibitor, T-cell surface glycoprotein CD8 inhibitor, T-cell transcription factor NFAT modulator, Tau aggregation inhibitor, Tau deposition inhibitor, Tec tyrosine kinase inhibitor, TGF beta 1 ligand inhibitor, TGF beta 3 ligand inhibitor, TGF beta 3 ligand modulator, TGF beta ligand inhibitor, TGF beta receptor agonist, TGF beta receptor antagonist, TGF-beta activated kinase-1 inhibitor, TGF-beta type II receptor antagonist, TGF-beta type II receptor modulator, TGF-beta type III receptor antagonist, Thromboxane A2 antagonist, Thromboxane synthetase inhibitor, Thymic stromal lymphopoietin ligand, Thymic stromal lymphopoietin ligand inhibitor, Thymic stromal lymphopoietin ligand modulator, Thymic stromal lymphopoietin receptor antagonist, Thymic stromal lymphopoietin receptor modulator, TLR agonist, TLR modulator, TLR-2 agonist, TLR-2 antagonist, TLR-4 antagonist, TLR-6 agonist, TLR-7 antagonist, TLR-8 antagonist, TLR-9 agonist, TLR-9 antagonist, TNF agonist, TNF alpha ligand inhibitor, TNF alpha ligand modulator, TNF antagonist, TNF binding agent, TNF related apoptosis inducing ligand, TNF-like receptor-2 modulator, Tumor necrosis factor 15 ligand inhibitor, Topoisomerase IV inhibitor, TRAIL receptor agonist, Transcription factor inhibitor, Transcription factor modulator, Transthyretin modulator, Trk tyrosine kinase receptor inhibitor, TRP cation channel A1 inhibitor, TRP cation channel A1 modulator, TRP cation channel C1 inhibitor, TRP cation channel V1 antagonist, TRP cation channel V1 modulator, TRP cation channel V2 modulator, Tsl protein kinase inhibitor, Tubulin binding agent, Tubulin receptor antagonist, Tumor necrosis factor 13B receptor modulator, Tumor necrosis factor 13C receptor modulator, Tumor necrosis factor 14 ligand inhibitor, Tumor necrosis factor 15 ligand modulator, Tumor necrosis factor ligand inhibitor, Txk tyrosine kinase inhibitor, Tyk2 tyrosine kinase inhibitor, Tyk2 tyrosine kinase modulator, Type I IL-1 receptor antagonist, Type I TNF receptor antagonist, Type II TNF receptor modulator, Tyrosine phosphatase substrate 1 inhibitor, Ubiquitin inhibitor, Ubiquitin ligase modulator, Ubiquitin ligase stimulator, Ubiquitin thioesterase-4 inhibitor, Unspecified GPCR antagonist, Unspecified GPCR modulator, Unspecified ion channel inhibitor, Uteroglobin stimulator, V-set transmembrane domain protein 1 stimulator, Vascular cell adhesion protein 1 antagonist, VEGF ligand inhibitor, VEGF receptor modulator, VEGF-2 receptor modulator, Vimentin inhibitor, Vitamin D3 receptor agonist, Wnt 5A ligand inhibitor, Wnt ligand modulator or YSK-4 protein kinase inhibitor.

### Kits

Provided herein are also kits that include a compound described herein, or a pharmaceutically acceptable salt, tautomer, stereoisomer, mixture of stereoisomers, prodrug, or deuterated analog thereof, and suitable packaging. In one embodiment, a kit further includes instructions for use. In one aspect, a kit includes a compound of Formula I (or any other Formula described herein), or a pharmaceutically acceptable salt, tautomer, stereoisomer, mixture of stereoisomers, prodrug, or deuterated analog thereof, and a label and/or instructions for use of the compounds in the treatment of the indications, including the diseases or conditions, described herein.

Provided herein are also articles of manufacture that include a compound described herein or a pharmaceutically acceptable salt, tautomer, stereoisomer, mixture of stereoisomers, prodrug, or deuterated analog thereof in a suitable container. The container may be a vial, jar, ampoule, preloaded syringe, and intravenous bag.

### EXAMPLES

### Experimental Procedures

Reactions were performed at room temperature unless stated otherwise. When a compound whose synthesis is described herein was used at a larger scale than its reported synthesis, it is to be implicitly understood that more material had been prepared similarly.

Microwave reactions were performed in dedicated MW instruments in closed vials.

¹H NMR spectra were recorded at 250-600 MHz in d₆-DMSO unless stated otherwise.

Reactions performed above the boiling point of the solvent took place in sealed tubes or screw-cap vials.

“Dried” refers to drying an organic solution over Na₂SO₄, MgSO₄, or CaCl₂ and filtering off the drying agent.

“Degassed” refers to air-sensitive reactions being flushed with inert atmosphere via evacuation and back-filling or by bubbling inert atmosphere through the mixture for several minutes.

NaH was used as a 60% oil dispersion.

“Filtration” of mixtures refers to the removal/isolation of solid material from mixture by filtration through a paper filter, PFTE septum, or through a pad of celite. Additional solvent was used to wash the solid.

“Purification by SCX” refers to the loading of the material onto a SCX column, washing with MeOH, eluting with NH₃ in MeOH, and concentrating the fractions containing the product.

“Partitioned (A/B)” refers to the partitioning of a mixture between solvents A and B. OL (A/B) refers to the organic layer after partitioning the mixture between solvents A and B. AL (A/B) refers to the aq. layer after partitioning the mixture between solvents A and B.

Crude reaction mixtures after reductions with iron/zinc power and AcOH/NH₄Cl were typically filtered through celite before work-up of the filtrate.

Catalytic hydrogenations were performed using a catalyst such as 10% Pd/C and a H₂ balloon unless stated otherwise. The mixture after the reaction was typically filtered through celite to remove the catalyst.

Intermediates were obtained sufficiently pure for the next step from the procedures outlined in the specification.

Some of the final compounds (Examples) were obtained in pure form and the yield provided accordingly while others were obtained as DMSO solutions for which the concentration and volume are specified.

### HPLC Methods

Several Intermediates and final compounds (Examples) were purified using the HPLC methods listed below.

| Column | Eluent | Flow rate |
|---|---|---|
| XBridge Prep C18 OBD, | A: 50 mM aq. NH₄HCO₂, B: ACN, 10-100% | 30 mL/min |
| 150 × 19 mm 5 μm | ACN | |
| XTerra ® RP-18 OBD, 150 × 19 | A: 0.1% aq. NH₄HCO₂, B: ACN, 10-100% | 30 mL/min |
| mm 5 μm | ACN | |
| PRINCETONE ULTIMA | A: 0.05% aq. HCO₂H, B: ACN | 16 mL/min |
| C18 250 × 20 mm, 5 μm | 0 min 20% B, 1 min 20% B, 10 min 70% B, | |
| | 10.1 min 99% B, 12 min 99% B, 12.1 min, 20% | |
| | B, 15 min 20% B 12.1/20, 15/20 | |
| PRINCETONE ULTIMA | A: 0.1% aq. HCO₂H, B: ACN | 16 mL/min |
| C18 250 × 20 mm, 5 μm | 0 min 20% B, 2 min 20% B, 10 min 50% B | |
| LUNA OMEGA C18 | A: 10 mM aq. NH₄HCO₂, B: ACN | 18 mL/min |
| 250 × 21.2 mm, 5 μm | 0 min 40% B, 1 min 40% B, 10 min 85% B, | |
| | 10.1 min 99% B, 12 min 99% B, 12.1 min 40% | |
| | B, 15 min 40% B | |
| COGENT C18 250 × 21.2 mm, | A: 10 mM aq. NH₄HCO₂, B: ACN | 16 mL/min |
| 5 μm | 0 min 30% B, 2 min 30% B, 10 min 50% B | |
| COGENT C18 250 × 21.2 mm, | A: 10 mM aq. NH₄HCO₂, B: ACN | 20 mL/min |
| 5 μm | 0 min 30% B, 1 min 30% B, 10 min 80% B, | |
| | 10.1 min 99% B, 12 min 99% B, 12.1 min 30% | |
| | B, 15 min 30% B | |
| LUNA C18 150 × 21.2 mm, 5 | A: 10 mM aq. NH₄HCO₂, B: ACN | 18 mL/min |
| μm | 0 min 40% B, 1 min 40% B, 10 min 80% B, | |
| | 10.1 min 99% B, 12 min 99% B, 12.1 min 40% | |
| | B, 15 min 40% B | |
| X-SELECT PHENYL | A: 10 mM aq. NH₄HCO₂, B: ACN | 16 mL/min |
| HEXYL 250 × 19 mm, 5 μm | 0 min 30% B, 1 min 30% B, 10 min 55% B | |
| X-SELECT PHENYL | A: 10 mM aq. NH₄HCO₂, B: ACN | 18 mL/min |
| HEXYL 250 × 19 mm, 5 μm | 0 min 55% B, 1 min 55% B, 10 min 70% B, 15 | |
| | min 90% B, 15.1 min 100% B, 19 min 100% B, | |
| | 19.1 min 55% B | |
| AZZOTA 250 × 20mm, 10 μm | A: 10 mM aq. NH₄HCO₂, B: ACN | 18 mL/min |
| | 0 min 40% B, 1 min 40% B, 10 min 85% B, | |
| | 10.1 min 99% B, 12 min 99% B, 12.1 min 40% | |
| | B, 15 min 40% B | |
| AZZOTA 250 × 20 mm, 5 μm | A: 10 mM aq. ABC, B: ACN | 17 mL/min |
| | 0 min 30% B 1 min 30% B, 10 min 85% B | |
| PRINCETONE ULTIMA | A: 0.1% aq. HCO₂H, B: ACN | 17 mL/min |
| C18 250 × 20 mm, 10 μm | 0 min 10% B, 1 min 10% B, 10 min 90% B, | |
| | 10.1 min 99% B, 13 min 99% B, 13.1 min 10% | |
| | B, 16 min 10% B | |
| XSELECT CSH C18 250 × 19 | A: 0.1% aq. HCO₂H, B: ACN | 17 mL/min |
| mm 5 μm | 0 min 10% B, 1 min 10% B, 10 min 90% B, | |
| | 10.1 min 99% B, 13 min 99% B, 13.1 min 10% | |
| | B, 16 min 10% B | |
| YMC-PACK-ODS-AQ C18 | A: 0.1% aq. HCO₂H, B: ACN | 15 mL/min |
| 250 × 20 mm 5 μm | 0 min 25% B, 2 min 25% B, 10 min 35% B | |
| HICHROME C18 250 × 20 | A: 10 mM aq. ABC, B: ACN | 18 mL/min |
| mm 5 μm | 0 min 40% B, 2 min 40% B, 10 min 80% B | |
| X SELECTC18 250 × 19 mm | A: 10 mM aq. ABC, B: ACN | 16 mL/min |
| 5 μm | 0 min 40% B, 1 min 40% B, 10 min 80% B, 11 | |
| | min 80% B, 11.1 min 99% B, 15 min 99% B, | |
| | 15.1 min 40% B, 19 min 40% B | |

### LC/MS Methods

The compounds of the disclosure (Examples) were characterized by the LC/MS methods listed below.

| Method | Column and eluent | Gradient | Flow rate | |
|---|---|---|---|---|
| 1 | Agilent Poroshell 120 SB-C18 4.6 × 30 | 0 min 1% B, 1.5 min | 3 | mL/min |
| | mm 2.7 μm operated at 60° C. | 100% B, 1.73 min | | |
| | A: 0.1% aq. HCO₂H | 100% B | | |
| | B: 0.1% HCO₂H in ACN | | | |
| 2 | Acquity BEH C18 50 × 2.1 mm 1.7 μm | 0 min 3% B, 0.4 min | 0.6 | mL/min |
| | operated at 35° C. | 3% B, 2.5 min 98% B, | | |
| | A: 0.05% aq. HCO₂H | 3.5 min 98%, 3.6 min | | |
| | B: 0.05% HCO₂H in ACN | 3% B, 4 min 3% B | | |
| 3 | Acquity BEH C18 50 × 2.1 mm 1.7 μm | 0 min 3% B, 2.5 min | 0.6 | mL/min |
| | operated at 35° C. | 3% B, 7.5 min 98% B, | | |
| | A: 0.05% aq. HCO₂H | 9.6 min 3% B, 10 min | | |
| | B: 0.05% HCO₂H in ACN | 3% B | | |
| 4 | Acquity BEH C18 100 × 2.1 mm 1.7 | 0 min 3% B, 8.5 min | 0.55 | mL/min |
| | μm operated at 50° C. | 100% B, 9 min 100% | | |
| | A: 0.05% aq. HCO₂H | B, 9.5 min 3% B, 10 | | |
| | B: 0.05% HCO₂H in ACN | min 3% B | | |
| 5 | Acquity BEH C18 50 × 2.1 mm 1.7 μm | 0 min 3% B, 0.4 min | 0.6 | mL/min |
| | operated at 35° C. | 3% B, 2.5 min 98% B, | | |
| | A: 0.05% aq. TFA | 3.5 min 98% B, 3.6 | | |
| | B: 0.05% TFA in ACN | min 3% B, 4 min 3% | | |
| | | B | | |
| 6 | Acquity BEH C18 50 × 2.1 mm 1.7 μm | 0 min 3% B, 2.5 min | 0.6 | mL/min |
| | operated at 35° C. | 3% B, 7.5 min 98% B, | | |
| | A: 0.05% aq. TFA | 9.5 min 98% B, 9.6 | | |
| | B: 0.05% TFA in ACN | min 3% B, 10 min 3% | | |
| | | B | | |
| 7 | Acquity BEH C18 100 × 2.1 mm 1.7 | 0 min 3% B, 16 min | 0.45 | mL/min |
| | μm operated at 50° C. | 100% B, 18 min 100% | | |
| | A: 0.05% aq. TFA | B, 18.5 min 3% B, 20 | | |
| | B: 0.05% TFA in ACN | min 3% B | | |
| 8 | Xbridge C18 75 × 4.6 mm 3.5 μm | 0 min 5% B, 0.5 min | 1.3 | mL/min |
| | operated at 35° C. | 5% B, 1 min 15% B, 4 | | |
| | A: 10 mM aq. NH₄HCO₃ | min 98% B, 7 min | | |
| | B: ACN | 98% B, 7.5 min 5% B, | | |
| | | 8 min 5% B | | |
| 9 | X Bridge C18 150 × 4.6 mm 3.5 μm | 0 min 5% B, 1 min 5% | 1.0 | mL/min |
| | operated at 35° C. | B, 3 min 15% B, 7 | | |
| | A: 10 mM aq. NH₄HCO₃ | min 55% B, 11 min | | |
| | B: ACN | 98% B, 16 min 98% | | |
| | | B, 16.1 min 5% B, 20 | | |
| | | min 5% B | | |
| 10 | X SELECT C18 150 × 4.6 mm 3.5 μm | 0 min 5% B, 1 min 5% | 1.0 | mL/min |
| | operated at RT | B, 3 min 15% B, 7 | | |
| | A: 10 mM aq. NH₄HCO₃ | min 55% B, 11 min | | |
| | B: ACN | 98% B, 16 min 98% | | |
| | | B, 16.1 min 5% B, 20 | | |
| | | min 5% B | | |
| 11 | Acquity UPLC HSS T3 50 × 2.1 mm | 0 min 1% B, 0.5 min | 0.7 | mL/min |
| | 1.8 μm operated at 30° C. | 6% B, 1 min 6% B, | | |
| | A: 10 mM aq. NH₄OAc + 0.1% | 2.6 min 95% B, 3.8 | | |
| | HCO₂H | min 95% B, 3.81 min | | |
| | B: 0.1% HCO₂H in ACN | 1% B, 4.8 min 1% B | | |
| 12 | Acquity UPLC HSS T3 50 × 2.1 mm | 0 min 5% B, 0.9 min | 1.2-1.3 | mL/min |
| | 1.8 μm operated at 60° C. | 95% B, 1.2 min 95% | | |
| | A: 10 mM aq. NH₄OAc + 0.1% | B, 1.4 min 5% B | | |
| | HCO₂H | | | |
| | B: 0.1% HCO₂H in ACN | | | |
| 13 | X Brigde BEH C18 (3 × 100) mm, | 0 min 5% A, 5 min | 1 | mL/min |
| | 2.5 μm operated at 35° C. | 98% A, 9 min 98% A, | | |
| | A: 0.05% TFA in ACN | 9.01 min 5% A, 12 | | |
| | B: 0.05% aq. TFA | min 5% A | | |
| 14 | ACQUITY UPLC BEH C18 | 0 min 10% A, 2.5 min | 0.4 | mL/min |
| | (2.1 × 100) mm, 1.7 μm operated at 60 | 10% A, 7.5 min 98% | | |
| | ° C. | A, 9.5 min 98% A, 9.6 | | |
| | A: 0.05% TFA in ACN | min 10% A, 10 min | | |
| | B: 0.05% aq. TFA | 10% A | | |
| 15 | YMC-Triart C18 ExRS (75 × 4.6 mm, | 0 min 5% B, 0.8 min | 1.0 | mL/min |
| | 3 μm) operated at 45° C. | 5% B, 2 min 25% B, 5 | | |
| | A: 10 mM aq. NH₄HCO₃ | min 90% B, 7 min | | |
| | B: 100% ACN | 95% B, 8.5 min 95% | | |
| | | B, 8.6 min 5% B, 10 | | |
| | | min 5% B | | |
| 16 | X-BRIDGE C8 (4.6 × 75 mm) 3.5 μm | 0 min 5% B, 3 min | 1.0 | mL/min |
| | operated at 50° C. | 98% B, 5 min 98% B, | | |
| | A: 0.05% aq. HCO₂H | 5.5 min 5% B, 6 min | | |
| | B: 0.05% HCO₂H in ACN | 5% B | | |
| 17 | Xbridge C18 (75 × 4.6 mm, 3 μm) | 0 min 5% B, 0.5 min | 1.0 | mL/min |
| | operated at 50° C. | 5% B, 1 min 15% B, 4 | | |
| | A: 10 mM aq. NH₄HCO₃ | min 98% B, 7 min | | |
| | B: ACN | 98% B, 7.5 min 5% B, | | |
| | | 8 min 5% B | | |
| 18 | Acquity BEH C18 (50 mm × 2.1 mm, | 0 min 5% B, 5 min | 0.6 | mL/min |
| | 1.7 μm) operated at 35° C. | 98% B, 9 min 98% B, | | |
| | A: 0.05% aq. HCO₂H | 9.01 min 5% D, 12 | | |
| | B: 0.05% TFA in ACN | min 5% B | | |
| 19 | ACQUITY UPLC BEH C18 | 0 min 10% A, 2.5 min | 0.4 | mL/min |
| | (2.1 × 100) mm, 1.7 μm operated at 45 | 10% A, 7.5 min 98% | | |
| | ° C. | A, 9.5 min 98% A, 9.6 | | |
| | A: 0.05% TFA in ACN | min 10% A, 10 min | | |
| | B: 0.05% aq. TFA | 10% A | | |
| 20 | ACQUITY UPLC BEH C18 | 0 min 10% A, 2.5 min | 0.4 | mL/min |
| | (2.1 × 100) mm, 1.7 μm operated at 35 | 10% A, 7.5 min 98% | | |
| | ° C. | A, 9.5 min 98% A, 9.6 | | |
| | A: 0.05% TFA in ACN | min 10% A, 10 min | | |
| | B: 0.05% aq. TFA | 10% A | | |
| 21 | ACQUITY UPLC BEH C18 | 0 min 10% A, 2.5 min | 0.55 | mL/min |
| | (2.1 × 100) mm, 1.7 μm operated at 50 | 10% A, 7.5 min 98% | | |
| | ° C. | A, 9.5 min 98% A, 9.6 | | |
| | A: 0.05% TFA in ACN | min 10% A, 10 min | | |
| | B: 0.05% aq. TFA | 10% A | | |
| 22 | ACQUITY UPLC BEH C18 | 0 min 10% A, 2.5 min | 0.65 | mL/min |
| | (2.1 × 100) mm, 1.7 μm operated at 35 | 10% A, 7.5 min 98% | | |
| | ° C. | A, 9.5 min 98% A, 9.6 | | |
| | A: 0.05% TFA in ACN | min 10% A, 10 min | | |
| | B: 0.05% aq. TFA | 10% A | | |
| 23 | X Bridge C18 (50 mm × 4.6 mm, 3.5 | 0 min 5% B, 0.5 min | 0.8 | mL/min |
| | μm) operated at 45° C. | 5% B , 5 min 98% B, | | |
| | A: 10 mM aq. NH₄HCO₃ | 7.5 min 98% B, 7.6 | | |
| | B: ACN | min 5% B, 9 min 5% | | |
| | | B | | |
| 24 | YMC-Triart C18 ExRS (50 × 2.1 mm, | 0 min 5% B, 5 min | 0.6 | mL/min |
| | 1.9 μm) operated at 35° C. | 98% B, 9 min 98% B, | | |
| | A: 0.05% aq. TFA | 9.01 min 5% B, 12 | | |
| | B: 0.05% TFA in ACN | min 5% B | | |
| 25 | X Bridge C18 50 × 4.6 mm 3.5 um | 0 min 2% B, 0.5 min | 0.8 | mL/min |
| | operated at 35° C. | 2% B, 3 min 98% B, 6 | | |
| | A: 10 mM aq. NH₄HCO₃ | min 98% B, 8 min 2% | | |
| | B: ACN | B | | |
| 26 | X Bridge C18 50 × 4.6 mm 3.5 μm | 0 min 5% B, 0.5 min | 1.3 | mL/min |
| | operated at 30° C. | 5% B, 1.0 min 15% B, | | |
| | A: 10 mM aq. NH₄HCO₃ | 4.0 min 98% B, 7.0 | | |
| | B: ACN | min 98% B, 7.5 min | | |
| | | 5% B, 8.0 min 5% B | | |
| 27 | X Bridge C18 50 × 4.6 mm 3.5 μm | 0 min 2% B, 0.5 min | 1.0 | mL/min |
| | operated at 45° C. | 2% B, 2.5 min 98% B, | | |
| | A: 10 mM aq. NH₄HCO₃ | 5 min 98% B, 5.1 min | | |
| | B: ACN | 2% B, 6 min 2% B | | |
| 28 | YMC Triart C18 (75 × 4.6 mm, 3 μm) | 0 min 5% B, 0.5 min | 1.3 | mL/min |
| | operated at 45° C. | 5% B, 1 min 15% B, 6 | | |
| | A: 5 mM aq. NH₄HCO₃ | min 55% B, 9 min | | |
| | B: ACN | 95% B, 12 min 95% | | |
| | | B, 13 min 5% B, 14 | | |
| | | min 5% B | | |
| 29 | YMC Triart C18 (75 × 2.1 mm, 1.9 μm) | 0 min 3% B, 0.4 min | 0.6 | mL/min |
| | operated at 35° C. | 3% B, 2.5 min 98% B, | | |
| | A: 0.05% aq. TFA | 3.5 min 98% B, 3.6 | | |
| | B: 0.05% TFA in ACN | min 3% B, 4 min 3% | | |
| | | B | | |
| 30 | YMC Triart C18 (50 × 2.1 mm, 1.9 μm) | 0 min 3% A, 2.5 min | 0.6 | mL/min |
| | operated at 60° C. | 3% A, 7.5 min 98% A, | | |
| | A: 0.05% aq. TFA | 9.5 min 98% A, 9.6 | | |
| | B: 0.05% TFA in ACN | min 3% A, 10 min 3% | | |
| | | A | | |
| 31 | YMC Triart C18 (50 × 2.1 mm, 1.9 μm) | 0 min 3% A, 0.4 min | 0.6 | mL/min |
| | operated at 60° C. | 3% A, 2.5 min 98% A, | | |
| | A: 0.05% aq. TFA | 3.5 min 98% A, 3.6 | | |
| | B: 0.05% TFA in ACN | min 3% A, 4 min 3% | | |
| | | A | | |
| 32 | YMC Triart C18 (50 × 4.6 mm, 1.9 μm) | 0 min 5% A, 0.5 min | 1.0 | mL/min |
| | operated at 60° C. | 5% A, 3 min 95% A, 6 | | |
| | A: 0.05% TFA in ACN | min 95% A, 6.1 min | | |
| | B: 0.05% aq. TFA | 5% A, 8 min 5% A | | |
| 33 | X Bridge C18 75 × 4.6 mm 3.5 μm | 0 min 5% B, 0.5 min | 1.0 | mL/min |
| | operated at 45° C. | 5% B, 1 min 15% B, 6 | | |
| | A: 5 mM aq. NH₄HCO₃ | min 55% B, 9 min | | |
| | B: ACN | 95% B, 12 min 95% | | |
| | | B, 13 min 5% B, 14 | | |
| | | min 5% B | | |
| 34 | X Bridge C18 75 × 4.6 mm 3.5 μm | 0 min 5% B, 0.5 min 5 | 1.3 | mL/min |
| | operated at 35° C. | % B, 1.0 min 15% B, | | |
| | A: 10 mM aq. NH₄HCO₃ | 4.0 min 98% B, 7.0 | | |
| | B: ACN | min 98% B, 7.5 min | | |
| | | 5% B, 8.0 min 5% B | | |
| 35 | X Brigde BEH C18 (3 × 100) mm, | 0 min 3% A, 4 min | 1 | mL/min |
| | 2.5 μm operated at 35° C. | 98% A, 5 min 98% A, | | |
| | A: 0.05% TFA in ACN | 5.01 min 3% A, 6 min | | |
| | B: 0.05% aq. TFA | 3% A | | |
| 36 | X Bridge C18 (100 mm × 3 mm, 2.5 | 0 min 3% A, 4 min | 1.0 | mL/min |
| | μm) operated at 50° C. | 98% A, 5 min 98% A, | | |
| | A: 0.05% aq. TFA | 5.01 min 3% A, 6 min | | |
| | B: 0.05% TFA in ACN | 3% A | | |
| 37 | X Bridge C18 (75 mm × 4.6 mm, 3.5 | 0 min 5% B, 0.8 min | 1.0 | mL/min |
| | μm) operated at 45° C. | 5% B, 2 min 25% B, 5 | | |
| | A: 10 mM aq. NH₄HCO₃ | min 90% B, 7 min | | |
| | B: 100% ACN | 95% B, 8.6 min 5% B, | | |
| | | 10 min 5% B | | |
| 38 | Acquity BEH C18 100 × 2.1 mm 1.7 | 0 min 3% A, 1 min | 0.55 | mL/min |
| | μm operated at 60° C. | 10% A, 8.5 min 100% | | |
| | A: 0.05% TFA in ACN | A, 9 min 100% A, 9.5 | | |
| | B: 0.05% aq. TFA | min 3% A, 10 min 3% | | |
| | | A | | |
| 39 | Acquity BEH C18 100 × 2.1 mm 1.7 | 0 min 3% A, 8 min | 0.45 | mL/min |
| | μm operated at 60° C. | 100% A, 9 min 100% | | |
| | A: 0.05% aq. TFA | A, 9.5 min 3% A, 10 | | |
| | B: 0.05% TFA in ACN | min 3% A | | |
| 40 | Acquity BEH C18 100 × 2.1 mm 1.7 | 0 min 10% B, 2.5 min | 0.4 | mL/min |
| | μm operated at 45° C. | 10% B, 7.5 min 98% | | |
| | A: 0.05% aq. TFA | B, 9.5 min 98% B, 9.6 | | |
| | B: 0.05% TFA in ACN | min 10% B, 10 min | | |
| | | 10% B | | |
| 41 | Agilent Poroshell 120 SB-C18 | 0 min 1% B, 1.5 min | 3 | mL/min |
| | 4.6 × 30 mm 2.7 μm operated at 45° C. | 100% B, 2.2 min | | |
| | A: 0.1% aq HCO₂H | 100% B | | |
| | B 0.1% HCO₂H in ACN | | | |
| 42 | Waters HSS T3 1.8 μm, 1.0 × 50 mm | 0 min 10% B, 0.1 min | 0.475 | mL/min |
| | column operated at 50° C. | 10% B, 0.6 min 95% | | |
| | A: 0.01% aq HCO₂H | B, 1.5 min 95% B, | | |
| | B 0.01% HCO₂H in AC | 1.51 min 10% B | | |

Abbreviations
ABPR = automated back pressure regulator
ACN = acetonitrile
AcOH = acetic acid
AdBrettPhos Pd G3 = [2-(di-1-adamantyl-
phosphino)-2′,4′,6′-tri-iso-propyl-3,6-
dimethoxybiphenyl][2-(2′-amino-1,1′-
biphenyl)]palladium(II) methanesulfonate
aq = aqueous
Boc = tert-butoxycarbonyl
Boc₂O = di-tert-butyl dicarbonate
BOP = (benzotriazol-1-
yloxy)tris(dimethylamino)-phosphonium
hexafluorophosphate
B₂Pin₂ = 4,4,4′,4′,5,5,5′,5′-octamethyl-2,2′-
bi(1,3,2-dioxaborolane)
Brettphos Pd G3 = [(2-Di-cyclo-
hexylphosphino-3,6-dimethoxy-2′,4′,6′-tri-iso-
propyl-1,1′-biphenyl)-2-(2′-amino-1,1′-
biphenyl)]palladium(II) methanesulfonate
methanesulfonate
BrettPhos Pd G4 = [2′-(methylamino)-[1,1′-
biphenyl]-2-yl]palladio methanesulfonate di-
cyclo-hexyl[3,6-dimethoxy-2′,4′,6′-tris(propan-
2-yl)-[1,1′-biphenyl]-2-yl]phosphane
brine = saturated aqueous sodium chloride
cataCXium Pd G4 = [di(adamantan-1-
yl)(butyl)phosphine](methanesulfonato-κO)[2′-
(methylamino)-2-biphenylyl]palladium
CBz = benzyloxycarbonyl
CBz-Cl = benzyl chloroformate
CDI = 1,1′-carbonyldiimidazole
DAST = diethylaminosulfur trifluoride
dba = dibenzylideneacetone
DCC = di-cyclo-hexylcarbodiimide
DCE = 1,2-dichloroethane
DCM = dichloromethane
DEA = diethylamine
DEAD = diethyl azodicarboxylate
Deoxo-Fluor = bis(2-methoxyethyl)aminosulfur
trifluoride
DIAD = di-iso-propyl azodicarboxylate
DIPEA = di-iso-propyl ethyl amine
DMAP = 4-(dimethylamino)pyridine
DME = 1,2-dimethoxyethane
DMF = dimethyl formamide
DMDCH = trans-N,N′-dimethyl-cyclo-hexane-
1,2-diamine
DMEDA = N1,N2-dimethylethane-1,2-diamine
DMP = Dess-Martin periodinane
DMS = dimethyl sulfide
DMSO = dimethyl sulfoxide
DPPF = 1,1′-bis(diphenylphosphino)ferrocene
EDC = N-(3-dimethylaminopropyl)-N′-
ethylcarbodi-imide
EtOAc = ethyl acetate
EtOH = ethanol
FC = flash chromatography on silica gel unless
stated otherwise from the eluent described in the
brackets
h = hour(s)
HATU = (1-[bis(dimethylamino)methylene]-
1H-1,2,3-triazolo[4,5-b]pyridinium 3-oxide
hexafluorophosphate
HBTU = N,N,N′,N′-tetramethyl-O-(1H-
benzotriazol-1-yl)uronium hexafluorophosphate
HFIP = 1,1,1,3,3,3-hexafluoro-2-propanol
HOBt = hydroxybenzotriazole
HPLC = high performance liquid
chromatography
Int./Ints. = intermediate/intermediates
IPA = iso-propyl alcohol
KOAc = potassium acetate
KOtBu = potassium tert-butoxide
LCMS = liquid chromatography-mass
spectrometry
LDA = lithium di-iso-propylamide
LiHMDS = lithium bis(trimethylsilyl)amide
mCPBA = m-chloro-perbenzoic acid
MeOH = methanol
2MeTHF = 2-methyl-tetrahydrofuran
MHz = megahertz
MS = molecular sieves
MsCl = methanesulfonyl chloride (mesyl
chloride)
MTBE = methyl tert-butyl ether
MW = microwave
NaOtBu = sodium tert-butoxide
NBS = N-bromosuccinimide
NMP = N-methyl-2-pyrrolidon
NMR = nuclear magnetic resonance
ON = overnight
Pd₂dba₃ = tris(dibenzylideneacetone)dipalladium(0)
PdDPPFCl₂-DCM = [1,1′-
bis(diphenylphosphino)-
ferrocene]dichloropalladium(II) DCM complex
Pd G3 SPhos = (2-Di-cyclo-hexylphosphino-
2′,6′-dimethoxybiphenyl) [2-(2′-amino-1,1′-
biphenyl)]palladium(II) methanesulfonate
Pd G3 tert-butyl-Xphos = methanesulfonato (di-
tert-butyl) phenylphosphino (2′-amino-1,1′-
biphenyl-2-yl) palladium(II)
ppm = parts per million
PyBOP = (benzotriazol-1-yloxy)tripyrrolidino-
phosphonium hexafluorophosphate
RT = room temperature
RU = response units
RuPhos = 2-di-cyclo-hexylphosphino-2′,6′-di-
iso-propoxybiphenyl
Ruphos Pd-G2 = Chloro(2-
dicyclohexylphosphino-2′,6′-diisopropoxy-1,1′-
biphenyl)[2-(2′-amino-1,1′-
biphenyl)]palladium(II)
sat. = saturated
SCX = strong cation exchange
SEM = 2-(trimethylsilyl)ethoxymethyl
SFC = supercritical fluid chromatography
SPR = Surface plasmon resonance
STAB = sodium triacetoxy borohydride
TBAF = tetra n-butyl ammonium fluoride
TBAI = tetra n-butyl ammonium iodide
TFA = trifluoro acetic acid
Tf₂O = trifluoromethanesulfonic anhydride
THF = tetrahydrofuran
TLC = thin layer chromatography
TMEDA = tetramethylethylenediamine
Ts = tosyl
TsOH = p-toluenesulfonic acid
T3P = propanephosphonic acid anhydride
VCD = Vibrational circular dichroism
XantPhos = 4,5-bis(diphenylphosphino)-9,9-
dimethylxanthene
XPhos = 2-di-cyclo-hexylphosphino-2′,4′,6′-tri-
iso-propylbiphenyl

### Synthesis Methods

The compounds of the present disclosure can be prepared in a number of ways well known to those skilled in the art of synthesis. The compounds of the disclosure could for example be prepared using the reactions and techniques outlined below together with methods known in the art of synthetic organic chemistry, or variations thereof as appreciated by those skilled in the art. Preferred methods include, but are not limited to, those described below. The reactions are carried out in solvents appropriate to the reagents and materials employed and suitable for the transformations being effected. Also, in the synthetic methods described below, it is to be understood that all proposed reaction conditions, including choice of solvent, reaction atmosphere, reaction temperature, duration of experiment and work-up procedures, are chosen to be conditions of standard for that reaction, which should be readily recognized by one skilled in the art. Not all compounds falling into a given class may be compatible with some of the reaction conditions required in some of the methods described. Such restrictions to the substituents which are compatible with the reaction conditions will be readily apparent to one skilled in the art and alternative methods can be used.

The compounds of the present disclosure or any intermediate could be purified, if required, using standard methods well known to a synthetic organist chemist, e.g. methods described in “Purification of Laboratory Chemicals”, 6^{th} ed. 2009, W. Amarego and C. Chai, Butterworth-Heinemann.

Starting materials are either known or commercially available compounds, or may be prepared by routine synthetic methods well known to a person skilled in the art.

Unless otherwise noted, reagents and solvents were used as received from commercial suppliers. The organic solvents used were usually anhydrous. The solvent ratios indicated refer to vol:vol unless otherwise noted. Thin layer chromatography was performed using Merck 60F254 silica-gel TLC plates. Visualization of TLC plates was performed using UV light (254 nm) or by an appropriate staining technique.

The compounds of the disclosure can be prepared according to the key bond formations as outlined below.

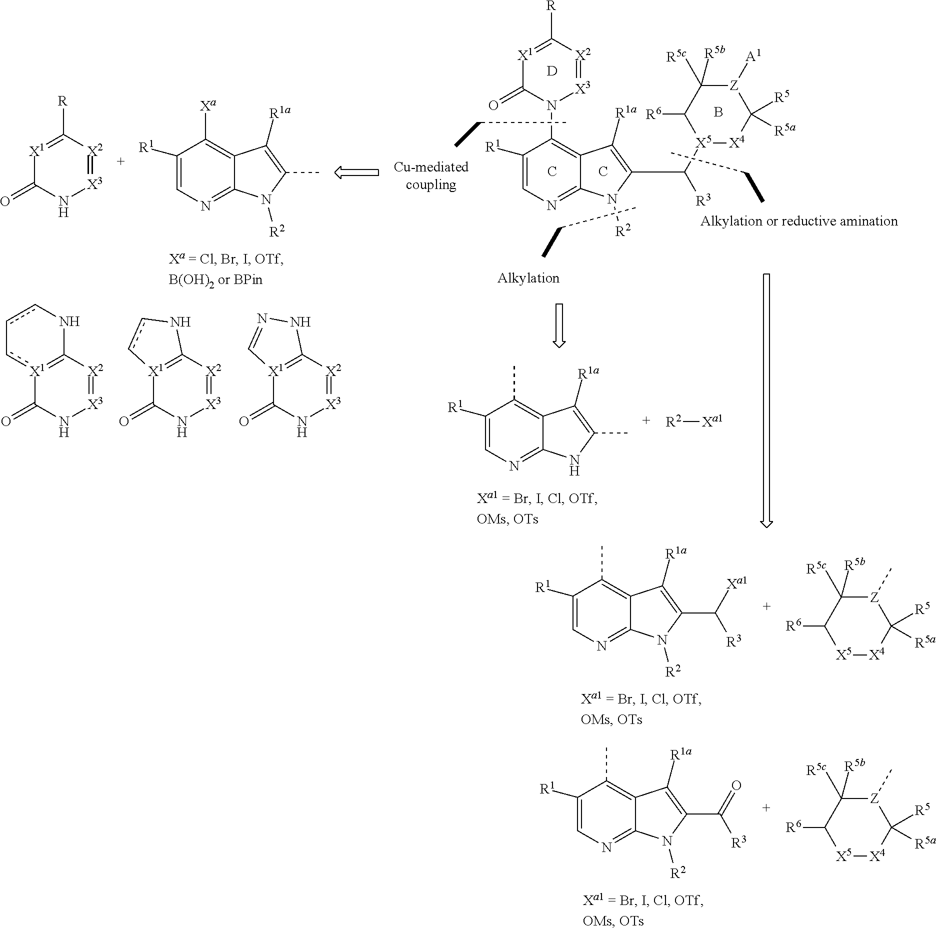

Linking A=CO to L-LBM (i.e. of R⁹) via an amide bond can be performed from the corresponding acid, an appropriate amine, and a suitable coupling agent for example under the conditions used to prepare Int. 1AF84.

Linking the A¹ and B rings can be achieved in palladium-mediated couplings reactions wherein either an amine (Z=N) is reacted with an aryl (pseudo)halide based on ring A¹ or by coupling ring B in which Z is a carbon atom linked to a boronic acid or boronate to aryl (pseudo)halide based on ring A¹. Such reactions can be performed as described for the syntheses of Ints. 1M51 and 1S3.

The bond connecting the B ring to the azaindole core (C—C) can be formed either by alkylation of the B ring amine or lactam with an alkyl (pseudo)halide linked to the azaindole core (C—C) or by reductive amination of the B ring amine to the ketone/aldehyde linked to the azaindole core (C—C). Such reactions can be performed as described for the synthesis of Examples 1d40/1d41 from Int. 1T1 or Examples 1m6 from Int. 1M1.

R² can be attached to the parent azaindole core (C—C) by alkylation with a suitable alkylation agent. Such reactions can be performed as described for the syntheses of Int. 1K3.

Attaching ring D to the azaindole core (C—C) can be done either by copper-mediated coupling of a boronic acid or boronate linked to the azaindole (C—C) core and the heteroaromatic ring D or by copper-mediated Ullmann reaction between an aryl (pseudo)halide derivative of the azaindole core (C—C) and the heteroaromatic ring D. Such reactions can be performed as described for the synthesis of Example 1af63 from Int. 1AFI or Example lab4 from 1AB4.

Additionally or alternatively, the compounds of the disclosure can be prepared according to the key bond formations as outlined below.

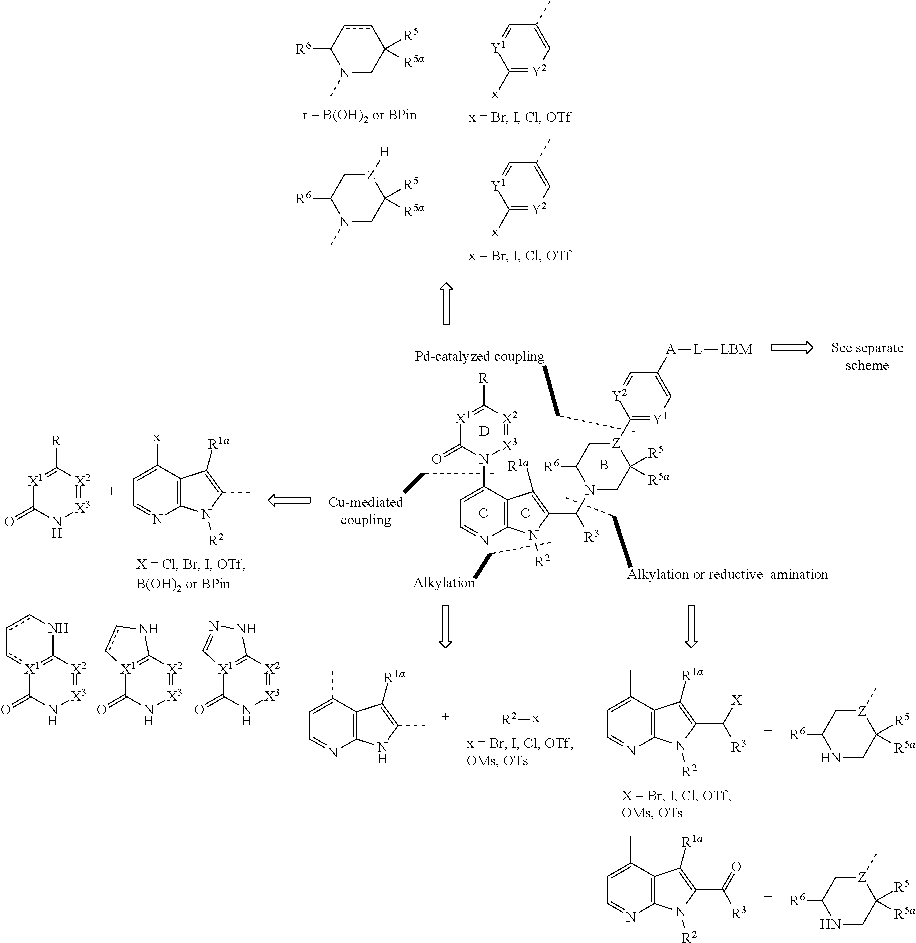

Linking A=CO to L-LBM via an amide bond can be performed from the corresponding acid, an appropriate amine, and a suitable coupling agent for example under the conditions used to prepare Int. 1AF84.

The bond connecting the B ring to the azaindole core (C—C) can be formed either by alkylation of the B ring amine or lactam with an alkyl (pseudo)halide liked to the azaindole core (C—C) or by reductive amination of the B ring amine to the ketone/aldehyde liked to the azaindole core (C—C). Such reactions can be performed as described for the synthesis of Examples 1d40/1d41 from Int. 1T1 or Examples 1m6 from Int. 1M1.

The bond connecting the B ring to the azaindole core (C—C) can be formed either by alkylation of the B ring amine or lactam with an alkyl (pseudo)halide liked to the azaindole core (C—C) or by reductive amination of the B ring amine to the ketone/aldehyde liked to the azaindole core (C—C). Such reactions can be performed as described for the synthesis of Examples 1d40/1d41 from Int. 1T1 or Examples 1m6 from Int. 1M1.

R² can be attached to the parent azaindole core (C—C) by alkylation with a suitable alkylation agent. Such reactions can be performed as described for the syntheses of Int. 1K3.

Attaching ring D to the azaindole core (C—C) can be done either by copper-mediated coupling of a boronic acid or boronate linked to the azaindole (C—C) core and the heteroaromatic ring D or by copper-mediated Ullmann reaction between an aryl (pseudo)halide derivative of the azaindole core (C—C) and the heteroaromatic ring D. Such reactions can be performed as described for the synthesis of Example 1af63 from Int. 1AFI or Example lab4 from 1AB4.

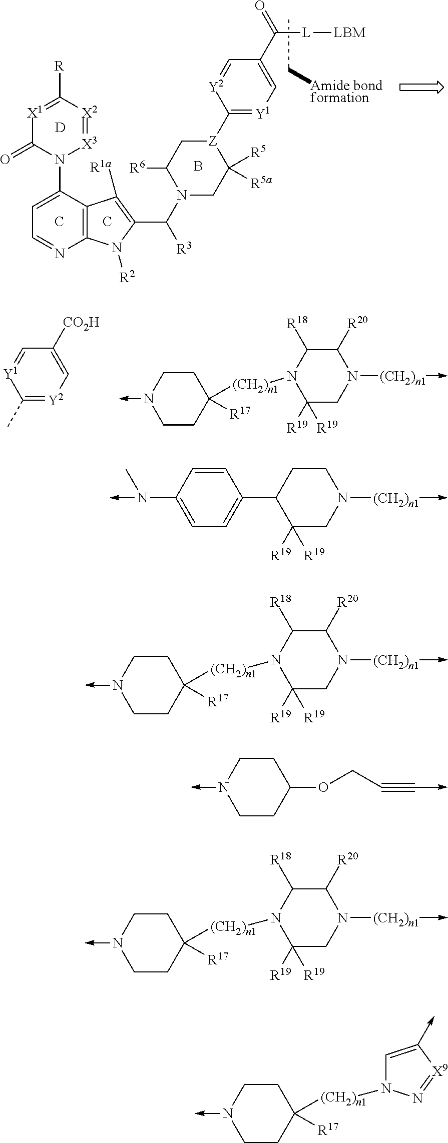

Compounds in which A=CO the -L- fragment can be introduced via amide bond formation. The prerequisite acids can for example be prepared as described herein for Int. 1M60 from Example 1m56.

The synthesis Int. 3E274 exemplifies a precursor in which the amide bond linking CO to L-LBM is formed from the prerequisite substrates Ints. 3E275/1AC4. The specification exemplifies L-LBM fragments such as Int. 3E68 and their coupling to give the compounds of the disclosure such as described for the reaction of Ints. 3E171/3E68 to give Example 3e39.

Compounds in which the LBM motif is linked to the rest of the molecule via a triple bond or a C—C to a five-membered aromatic ring can be prepared via cross-coupling reactions from precursors like Ints. 3E240/3B1.

Compounds in which the LBM motif is linked to the rest of the molecule via a methylene linker can be prepared by reductive amination such as described herein for Int. 3E2. Compounds in which the LBM is linked to the rest of the molecule via the 4-position of a piperidine ring can be prepared from Ints. like 3D1.

The LBM motif can be functionalized with Cl or F atoms such as in Ints. 3E50, 3E61, and 3E131.

The LBM motif can be functionalized on the benzimidazolone nitrogen atom not bearing the piperidine-2,6-dione unit by functionalized alkyl groups such as (CH₂)₂OH, (CH₂)₂OMe, or CH₂CF₃ as in Ints. 3E13, 3E159, and 3E106. The LBM motif can be functionalized on the benzimidazolone nitrogen atom not bearing the piperidine-2,6-dione unit by alkyl groups like methyl or cyclo-propyl as in Ints. 3E3, and 3E20.

Compounds in which the LBM motif is an oxindole or benzoxazole can be prepared from Ints. 3E288 and 3E257.

### Example 1—Preparation of Intermediates

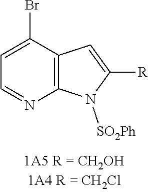

4-Bromo-1-(phenyl-sulfonyl)-1H-pyrrolo[2,3-b]pyridine-2-carbaldehyde (0.27 g) and NaBH₄ (33 mg) were stirred 0.5 h in THF/MeOH (9:1, 5 mL) at 0° C. The OL (water/EtOAc) was washed with brine, dried, and concentrated to give Int. 1A5 (4-bromo-1-(phenylsulfonyl)-1H-pyrrolo[2,3-b]pyridin-2-yl)methanol (281 mg). Int. 1A5 (0.24 g) and MsCl (0.052 mL) was stirred 0.5 h in DCM/Et₃N (29:1, 5.2 mL). MsCl (0.014 mL) was added and stirring continued 0.5 h. The OL (water/DCM) was washed with brine, dried, concentrated, and purified by FC (heptane/EtOAc 1:0 to 1:1) to give Int. 1A4 4-bromo-2-(chloromethyl)-1-(phenylsulfonyl)-1H-pyrrolo[2,3-b]pyridine (0.15 g).

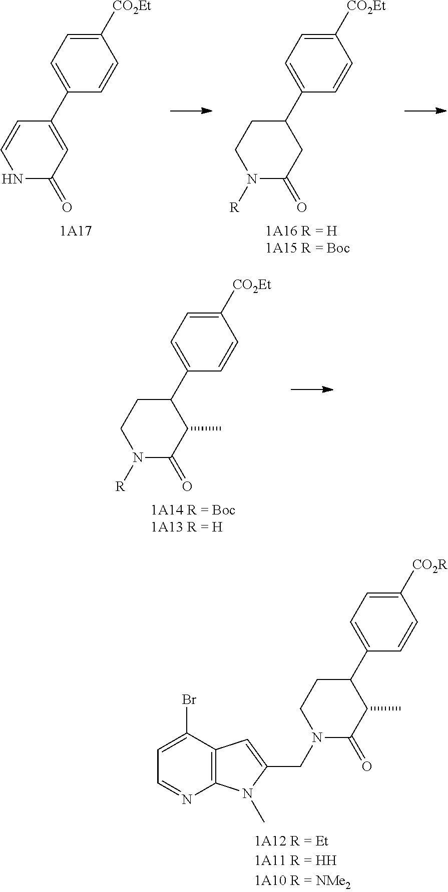

4-Bromopyridin-2 (1H)-one (1.0 g), ethyl 4-(4,4,5,5-tetramethyl-1,3,2-dioxaborolan-2-yl)benzoate (3.17 g), PdDPPFCl₂-DCM (0.42 g), and K₃PO₄ (3.66 g) were degassed in dioxane/water (10:1, 11 mL) and stirred 2 h at 110° C. under MW conditions. The mixture was filtered, concentrated, and purified by FC (DCM/MeOH 95:5) to give Int. 1A17 ethyl 4-(2-oxo-1H-pyridin-4-yl)benzoate (0.50 g). Int. 1A17 (3.5 g) was hydrogenated 48 h using 10% Pd/C (1.5 g) in EtOH/AcOH (4:1, 50 mL), filtered, concentrated, and purified by FC (hexane/EtOAc 7:3) to give Int. 1A16 ethyl 4-(2-oxopiperidin-4-yl)-benzoate (2.5 g). Int. 1A16 (0.50 g), Boc₂O (0.61 g), and DMAP (23 mg) were stirred ON in DCM/Et₃N (25:1, 21 mL) at 0° C. to RT ON. The OL (EtOAc/water) was washed with brine, dried, concentrated, and purified by FC (hexane/EtOAc 7:3) to give Int. 1A15 tert-butyl 4-(4-ethoxy-carbonylphenyl)-2-oxo-piperidine-1-carboxylate (0.40 g). Int. 1A15 (0.30 g) was stirred 0.5 h in 0.1M LiHMDS in THF (11 mL) at −78° C. CH₃I (0.16 mL) was added and stirring continued 2 h at −25 to −30° C. The OL (sat. aq. NH₄Cl/EtOAc) was washed with water and brine, dried, concentrated, and purified by FC (hexane/EtOAc 9:1) to give Int. 1A14 tert-butyl trans-4-(4-ethoxycarbonylphenyl)-3-methyl-2-oxo-piperidine-1-carboxylate (0.18 g). Int. 1A14 (0.60 g) was stirred ON in DCM/TFA (32:1, 20.6 mL) at 0° C. to RT and concentrated. The OL (10% MeOH in DCM/sat. aq. NaHCO₃) was dried, and concentrated to give Int. 1A13 ethyl 4-[trans-3-methyl-2-oxo-4-piperidyl]benzoate (0.38 g). Int. 1A13 (0.20 g) and NaH (28 mg) were stirred 0.5 h in THF (10 mL) at 0° C. to RT. Int. 1J1 (0.22 g) and TBAI (25 mg) were added and stirring continued ON. The OL (aq. citric acid/EtOAc) was dried and concentrated to give Ints. 1A12/1A11 ethyl 4-[trans-1-[(4-bromo-1-methyl-pyrrolo[2,3-b]pyridin-2-yl)methyl]-3-methyl-2-oxo-4-piperidyl]benzoate and 4-[trans-1-[(4-bromo-1-methyl-pyrrolo[2,3-b]pyridin-2-yl)methyl]-3-methyl-2-oxo-4-piperidyl]benzoic acid (0.30 g). 20 mg of this mixture, HNMe₂ (0.11 g, HCl salt), and HATU (0.25 g) were stirred ON in DMF/DIPEA (28.6:1, 10.4 mL). The mixture was combined with another batch prepared similarly on 30 mg scale, concentrated, and purified by FC (hexane/EtOAc 1:4) to give Int. 1A10 N,N-Dimethyl-4-[trans-1-[(4-bromo-1-methyl-pyrrolo[2,3-b]pyridin-2-yl)methyl]-3-methyl-2-oxo-4-piperidyl]benzamide (0.20 g).

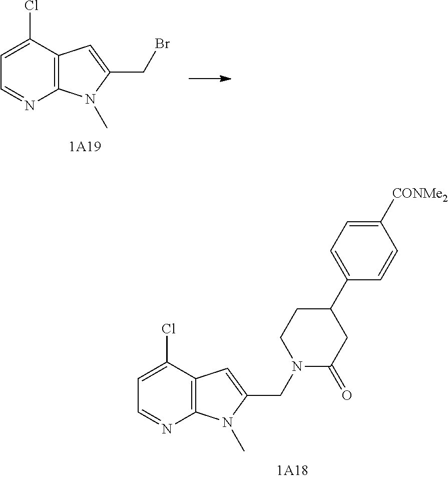

Int. 1A18 4-[1-[(4-Chloro-1-methyl-pyrrolo[2,3-b]pyridin-2-yl)methyl]-2-oxo-4-piperidyl]-N,N-dimethyl-benzamide was prepared similarly to Int. 1A10 from Int. 1A16 and 4-chloro-2-(chloromethyl)-1-methyl-1H-pyrrolo[2,3-b]pyridine (prepared similarly to Int. 1J1).

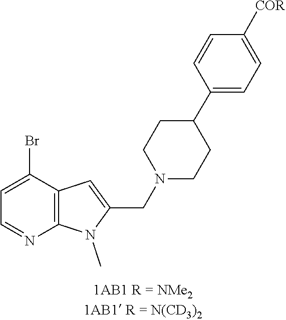

Int. 1J1 (8 g) and N,N-dimethyl-4-(4-piperidyl)benzamide (9.9 g, HCl salt) were stirred ON in DMF/DIPEA (0.7:1, 47 mL) at 0° C. to RT. The OL (water/EtOAc) was washed with brine, dried, concentrated, and purified by FC (hexane/EtOAc 1:3) to give Int. 1AB1 4-(1-((4-Bromo-1-methyl-1H-pyrrolo[2,3-b]pyridin-2-yl)methyl)piperidin-4-yl)-N,N-dimethylbenzamide (9 g). Int. 1AB1′ 4-(1-((4-bromo-1-methyl-1H-pyrrolo[2,3-b]pyridin-2-yl)methyl)piperidin-4-yl)-N,N-bis(methyl-d₃)benzamide was prepared similarly from HN(CD₃)₂.

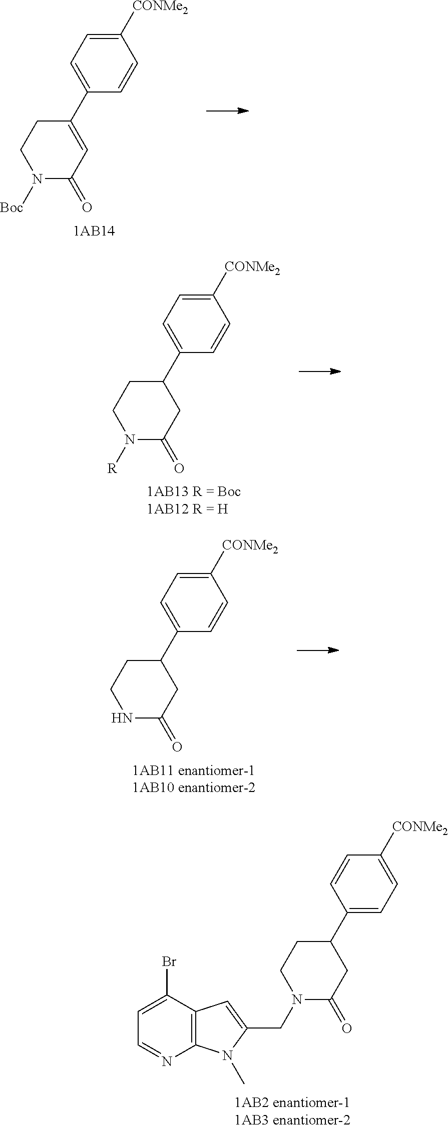

Na₂CO₃ (9.2 g), 1,1,1-trifluoro-N-phenyl-N-((trifluoromethyl)sulfonyl)methane-sulfonamide (10 g), and (4-(dimethylcarbamoyl)phenyl)boronic acid (5.5 g) were degassed in toluene/water (4:1, 250 mL) and stirred 3 h at 100° C. The mixture was filtered, concentrated, and purified by FC (hexane/EtOAc 2:3) to give Int. 1AB14 tert-butyl 4-[4-(dimethylcarbamoyl)phenyl]-6-oxo-2,3-dihydropyridine-1-carboxylate (5.1 g). Int. 1AB14 (4.4 g) was hydrogenated ON using 10% Pd/C (0.45 g) in MeOH (100 mL), filtered, and concentrated to give Int. 1AB13 tert-butyl 4-(4-(dimethylcarbamoyl)-phenyl)-2-oxopiperidine-1-carboxylate (3.9 g). Int. 1AB13 (8.1 g) was stirred ON in DCM/4M HCl in dioxane (4.2:1, 124 mL) and concentrated. The OL (sat. aq. NaHCO₃/10% MeOH in DCM) was dried and concentrated to give Int. 1AB12 N,N-dimethyl-4-(2-oxo-piperidin-4-yl)-benzamide (2.9 g). This material was resolved by SFC using a Chiralpak IG 250×25 5 μm column operated at 30° C. and an eluent of 50% CO₂ and 50% MeOH (100 g/min) and a back pressure of 100 bar to give Int. 1AB11 (0.93 g, first peak) and Int. 1AB12 (0.91 g, second peak). The absolute configurations of these compounds were not determined. Int. 1AB12 (95 mg) and NaH (20 mg) were stirred 0.25 h in DMF (1 mL). Int. 1J1 (0.10 g) was added and stirring continued 0.5 h. The OL (DCM/water) was washed with water and brine, dried, concentrated, and triturated in ACN to give Int. 1AB3 (S)-4-(1-((4-Bromo-1-methyl-1H-pyrrolo[2,3-b]pyridin-2-yl)methyl)-2-oxopiperidin-4-yl)-N,N-dimethylbenzamide or (R)-4-(1-((4-bromo-1-methyl-1H-pyrrolo[2,3-b]pyridin-2-yl)methyl)-2-oxopiperidin-4-yl)-N,N-dimethyl-benzamide (31 mg). Int. 1AB2 (S)-4-(1-((4-Bromo-1-methyl-1H-pyrrolo[2,3-b]pyridin-2-yl)methyl)-2-oxopiperidin-4-yl)-N,N-dimethylbenzamide or (R)-4-(1-((4-bromo-1-methyl-1H-pyrrolo[2,3-b]pyridin-2-yl)methyl)-2-oxopiperidin-4-yl)-N,N-dimethyl-benzamide (35 mg) was prepared similarly from Ints. 1AB11/1J1 (95 mg/0.10 g). The absolute configurations of these compounds were not determined.

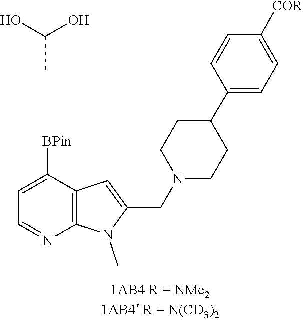

Int. 1ABI (0.15 g), B₂Pin₂ (0.13 g), KOAc (81 mg), and PdDPPFCl₂-DCM (27 mg) were degassed in dioxane (3 mL) and stirred at 90° C., filtered and concentrated to give Ints. 1AB4/1AB4′ N,N-Dimethyl-4-[1-[[1-methyl-4-(4,4,5,5-tetramethyl-1,3,2-dioxaborolan-2-yl)pyrrolo[2,3-b]pyridin-2-yl]methyl]-4-piperidyl]benzamide and [2-[[4-[4-(dimethylcarbamoyl)phenyl]-1-piperidyl]methyl]-1-methyl-pyrrolo[2,3-b]pyridin-4-yl]boronic acid and N,N-bis(methyl-d₃)-4-(1-((1-methyl-4-(4,4,5,5-tetramethyl-1,3,2-dioxaborolan-2-yl)-1H-pyrrolo[2,3-b]pyridin-2-yl)methyl)piperidin-4-yl)benzamide and (2-((4-(4-(bis-(methyl-d₃)carbamoyl)phenyl)piperidin-1-yl)methyl)-1-methyl-1H-pyrrolo[2,3-b]pyridin-4-yl)boronic acid.

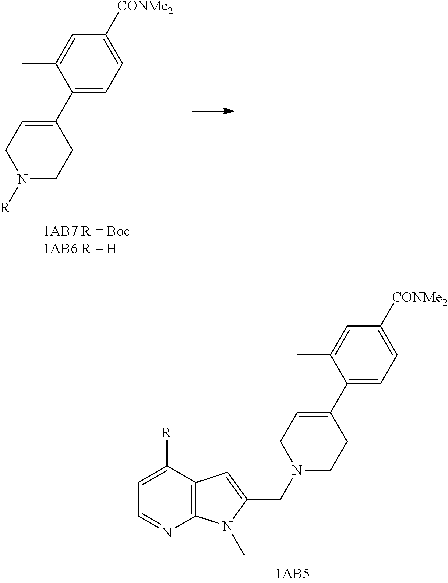

4-Bromo-N,N,3-trimethylbenzamide (9.0 g), tert-butyl 4-(4,4,5,5-tetramethyl-1,3,2-dioxaborolan-2-yl)-3,6-dihydro-2H-pyridine-1-carboxylate (15.2 g), PdDPPFCl₂-DCM (0.67 g), and Na₂CO₃ (9.62 g) were degassed in dioxane (180 mL) and stirred ON at 90° C. The mixture was filtered, concentrated, and purified by FC (hexane/EtOAc 2:3) to give Int. 1AB7 tert-butyl 4-[4-(dimethylcarbamoyl)-2-methyl-phenyl]-3,6-dihydro-2H-pyridine-1-carboxylate (11.0 g). Int. 1AB7 (0.6 g) was stirred ON in DCM/TFA (30:1, 15.5 mL), concentrated, and triturated in Et₂O to give Int. 1AB6 N,N,3-trimethyl-4-(1,2,3,6-tetrahydropyridin-4-yl)benzamide (TFA salt). Ints. 1AB6/1AB8 (0.23 g, TFA salt/0.1 g) were stirred ON in DCE/DIPEA (11.4:1, 5.4 mL) at 0° C. to RT. STAB (26 mg) was added and stirring continued 2 h at 0° C. to RT. The OL (water/DCM) was dried, concentrated, and purified by FC (MeOH/DCM 1:9) to give Int. 1AB5 4-(1-((4-Bromo-1-methyl-1H-pyrrolo[2,3-b]pyridin-2-yl)methyl)-1,2,3,6-tetrahydropyridin-4-yl)-N,N,3-trimethylbenzamide (0.10 g).

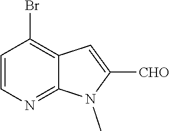

4-Bromo-1-methyl-1H-pyrrolo[2,3-b]pyridine (5.0 g) was stirred 2 h in 1.1M LDA in THF (33 mL) at −78° C. DMF (3.46 g in THF (20 mL)) was added and stirring continued ON at −78° C. to RT ON. The OL (sat. aq. NH₄Cl/EtOAc) was washed with water, brine, dried, concentrated, and triturated in EtOAc/pentane to give Int. 1AB8 4-Bromo-1-methyl-1H-pyrrolo[2,3-b]pyridine-2-carbaldehyde (3.1 g).

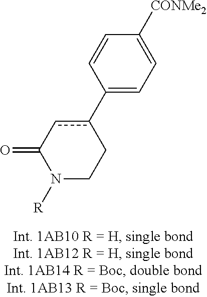

PdDPPFCl₂-DCM (1.1 g), tert-Butyl 6-oxo-4-(((trifluoromethyl)sulfonyl)oxy)-3,6-dihydropyridine-1 (2H)-carboxylate (10.2 g), (4-(dimethylcarbamoyl)phenyl)boronic acid (5.7 g), and Na₂CO₃ (9.4 g) were stirred 3 h in toluene/water (150/40 mL) at 100° C. The mixture was filtered. The filtrate was concentrated and purified by FC (EtOAc/Hexane 3:2) to give Int. 1AB14 tert-butyl 4-(4-(dimethyl-carbamoyl)-phenyl)-6-oxo-3,6-dihydropyridine-1 (2H)-carboxylate (5.2 g). Int. 1AB14 (5 g) and 10% Pd/C (2.5 g) were hydrogenated 48 h under a hydrogen pressure of 60 psi in EtOAc (100 mL). The mixture was filtered and concentration to give Int. 1AB13 tert-butyl 4-(4-(dimethylcarbamoyl)phenyl)-2-oxopiperidine-1-carboxylate (4.0 g). Int. 1AB13 (0.1 g) was stirred 12 h in DCM/4M HCl in dioxane (5/0.4 mL). The residue after concentration was triturated in Et₂O to give Int. 1AB10/1AB12 N,N-dimethyl-4-(2-oxopiperidin-4-yl)benzamide (78 mg).

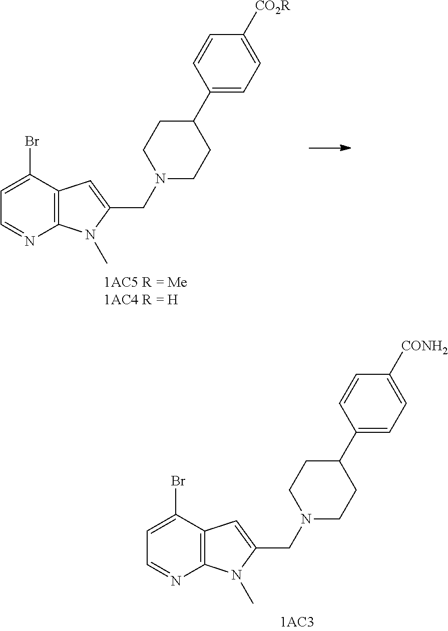

Int. 1AB8 (6.0 g) and methyl 4-(1-piperidin-4-yl)benzoate (7.1 g, HCl salt) were stirred 4 h in DCE/DIPEA (7.7:1, 113 mL). STAB (11.0 g) was added and stirring continued ON at 0° C. to RT. The OL (water/EtOAc) was washed with brine, dried, concentrated, and triturated in EtOAc/MeOH give Int. 1AC5 methyl 4-(1-((4-bromo-1-methyl-1H-pyrrolo[2,3-b]pyridin-2-yl)methyl)piperidin-4-yl)benzoate. Int. 1AC5 (0.4 g) and NaOH (0.18 g) were stirred ON in THF/MeOH (2:1, 9 mL), concentrated, and triturated in dilute aq. HCl to give Int. 1AC4 4-[1-[(4-bromo-1-methyl-pyrrolo[2,3-b]pyridin-2-yl)methyl]-4-piperidyl]-benzoic acid (0.38 g). Int. 1AC4 (0.5 g) and CDI (0.7 g) were stirred 2 h in DMF/Et₃N (30:1, 7.2 mL). 25% aq. NH₃ (1.51 mL) was added followed by water to precipitate a solid that was stirred ON in 1,1-dimethoxy-N,N-dimethylmethanamine (10 mL) to afford Int. 1AC3 4-(1-((4-bromo-1-methyl-1H-pyrrolo[2,3-b]pyridin-2-yl)methyl)piperidin-4-yl)benzamide (0.35 g).

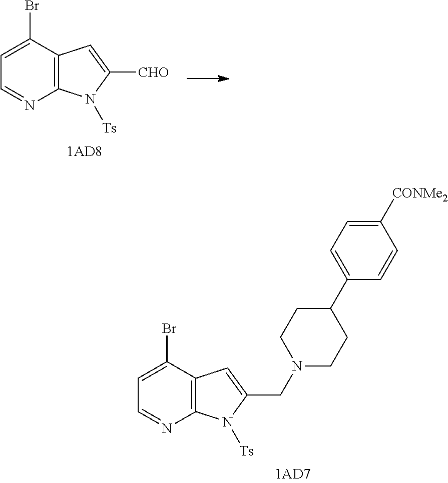

4-Bromo-1-tosyl-1H-pyrrolo[2,3-b]pyridine (1.0 g) was stirred 1 h in 0.6M LDA in THF (14.2 mL) at −78° C. DMF (1.3 mL) was added and stirring continued 2 h at −78° C. The OL (sat. aq. NH₄Cl/EtOAc) was dried, concentrated, and purified by FC (hexane/EtOAc 1:0 to 9:1) to give Int. 1AD8 4-bromo-1-(p-tolylsulfonyl)-pyrrolo[2,3-b]-pyridine-2-carbaldehyde (0.6 g). N,N-Dimethyl-4-(4-piperidinyl)-benzamide (1.4 g, HCl salt) and Int. 1AD8 (1.3 g) were stirred ON in DCE/DIPEA (20:1, 21 mL) at 0° C. to RT. STAB (1.1 g) was stirring continued 2 h at 0° C. to RT. The OL (water/DCM) was dried, concentrated, and purified by FC (DCM/MeOH 9:1) to give Int. 1AD7 4-[1-[[4-bromo-1-(p-tolyl-sulfonyl)-pyrrolo[2,3-b]pyridin-2-yl]methyl]-4-piperidyl]-N,N-dimethyl-benzamide (2.3 g).

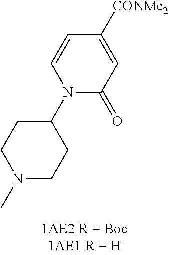

KOtBu (37 mg), tert-butyl 4-(p-tolylsulfonyloxy)piperidine-1-carboxylate (0.16 g), K₂CO₃ (83 mg), and N,N-dimethyl-2-oxo-1H-pyridine-4-carboxamide (50 mg) were degassed in DME (5 mL) and stirred ON at 120° C. The OL (water/MeOH/DCM) was dried and concentrated to give Int. 1AE2 tert-butyl 4-[4-(dimethyl-carbamoyl)-2-oxo-1-pyridyl]-piperidine-1-carboxylate. Int. 1AE2 (0.25 g) was stirred in DCM/TFA (8.6:1, 5.6 mL). The mixture was concentrated to give Int. 1AE1 N,N-Dimethyl-2-oxo-1-(piperidin-4-yl)-1,2-dihydropyridine-4-carboxamide (0.15 mg, TFA salt).

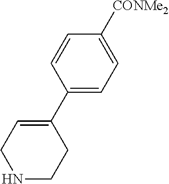

4-Bromo-N,N-dimethylbenzamide (25 g), tert-butyl 4-(4,4,5,5-tetramethyl-1,3,2-dioxaborolan-2-yl)-3,6-dihydropyridine-1 (2H)-carboxylate (40.7 g), Na₂CO₃ (34.5 g), and PdDPPFCl₂-DCM (2.24 g) were degassed in dioxane/water (7.7:1, 260 mL) and stirred at 90° C. ON and filtered. The OL (water/EtOAc) was washed with brine, dried, concentrated, and purified by FC (heptane/EtOAc 55:45) to give tert-butyl 4-(4-(dimethyl-carbamoyl)-phenyl)-3,6-dihydropyridine-1 (2H)-carboxylate (36.0 g). 5.0 g of this material was stirred ON in DCM/TFA (6.9:1, 46 mL) and concentrated. The OL (sat. aq. NaHCO₃/10% MeOH in DCM) was washed with brine, dried, concentrated, and triturated in Et₂O to give Int. 1AE7 4-(3,6-dihydro-2H-pyridin-4-yl)-N,N-dimethyl-benzamide (2.80 g).

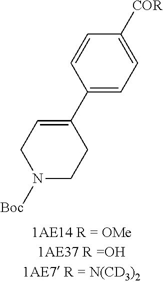

Methyl 4-bromobenzoic acid ester (10 g) tert-butyl 4-(4,4,5,5-tetramethyl-1,3,2-dioxaborolan-2-yl)-3,6-dihydropyridine-1 (2H)-carboxylate (14.4 g), NaHCO₃ (11.7 g), and PdDPPFCl₂-DCM (1.2 g) were degassed in dioxane (300 mL) and stirred at 100° C. ON. The OL (water/EtOAc) was dried, concentrated, and purified by FC (hexane/EtOAc 9:1) to give Int. 1AE14 tert-butyl 4-(4-(methoxycarbonyl)phenyl)-3,6-dihydropyridine-1 (2H)-carboxylate (11 g). 4.5 g of this material and LiOH—H₂O (3.0 g) were stirred 4 h in MeOH/THF/water (4:2:1, 35 mL) at 0° C. to RT. The mixture was partially concentrated and diluted with aq. citric acid to precipitate Int. 1AE37 4-(1-(tert-butoxycarbonyl)-1,2,3,6-tetrahydropyridin-4-yl)benzoic acid (3.9 g). Int. 1AE37 (4.0 g), HATU 7.52 g), and HN(CD₃)₂ (1.73 g, HCl salt) were stirred ON in DMF/DIPEA (2.5:1, 42 mL). The OL (EtOAc/water) was washed with brine, dried, concentrated, and purified by FC (hexane/EtOAc 1:4) to give tert-butyl 4-[4-[bis-(trideuteriomethyl)-carbamoyl]phenyl]-3,6-dihydro-2H-pyridine-1-carboxylate (3.5 g). 2.5 g of this material was stirred ON in DCM/TFA (1:1, 30 mL) at 0° C. to RT and concentrated. The OL (aq. NaHCO₃/10% MeOH in DCM) was dried, concentrated, and triturated in Et₂O/pentane to give Int. 1AE7′ N,N-Bis(methyl-d₃)-4-(1,2,3,6-tetrahydropyridin-4-yl)benzamide (1.7 g).

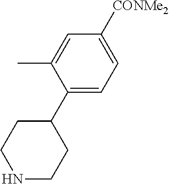

tert-Butyl 4-(4,4,5,5-tetramethyl-1,3,2-dioxaborolan-2-yl)-3,6-dihydropyridine-1 (2H)-carboxylate (33.7 g), 4-bromo-N,N,3-trimethylbenzamide (20.0 g), Na₂CO₃ (21.4 g), and PdDPPFCl₂-DCM (1.48 g) were degassed in dioxane/water (9:1, 200 mL) and stirred ON at 110° C. The mixture was filtered, concentrated, and purified by FC (heptane/EtOAc 1:1) to give tert-butyl 4-[4-(dimethylcarbamoyl)-2-methyl-phenyl]-3,6-dihydro-2H-pyridine-1-carboxylate (25 g). 15 g of this material was hydrogenated ON using 10% Pd/C (5.0 g) in MeOH (200 mL), filtered, and concentrated to give tert-butyl 4-[4-(dimethylcarbamoyl)-2-methyl-phenyl]-piperidine-1-carboxylate (14.0 g). 10 g of this material was stirred ON in DCM/TFA (1:1, 200 mL) at 0° C. to RT, concentrated, and triturated in Et₂O to give Int. 1AE8 N,N,3-trimethyl-(4-piperidin-4-yl)benzamide (9.2 g, TFA salt).

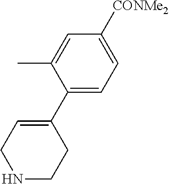

tert-Butyl 4-[4-(dimethylcarbamoyl)-2-methyl-phenyl]-3,6-dihydro-2H-pyridine-1-carboxylate (4.0 g) was stirred 0.5 h in 4M HCl in dioxane (40 mL), concentrated, and triturated in Et₂O to give Int. 1AE8′ N,N,3-trimethyl-4-(1,2,3,6-tetrahydropyridin-4-yl)benzamide (1.06 g, HCl salt).

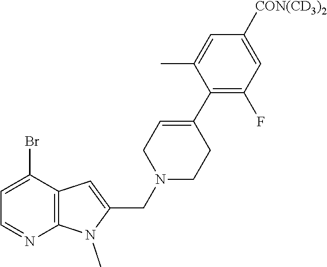

Ints. 1J1/1 (0.70 g/0.49 g, HCl salt) and KI (0.11 g) were stirred ON in DMF/DIPEA (8.3:1, 11.2 mL) at 0° C. to RT. The OL (EtOAc/water) was washed with brine, dried, concentrated, and purified by FC (EtOAc/hexane 7:3) to give Int. 1AE9 4-(1-((4-bromo-1-methyl-1H-pyrrolo[2,3-b]pyridin-2-yl)methyl)-1,2,3,6-tetrahydropyridin-4-yl)-3-fluoro-5-methyl-N,N-bis(methyl-d₃)benzamide (0.45 g).

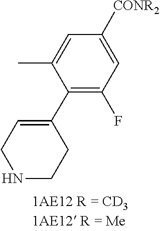

Methyl 4-bromo-3-fluoro-5-methyl-benzoate (20 g), tert-butyl 4-(4,4,5,5-tetramethyl-1,3,2-dioxaborolan-2-yl)-3,6-dihydropyridine-1 (2H)-carboxylate (27.5 g), K₂CO₃ (44.8 g), and PdDPPFCl₂-DCM (3.31 g) were degassed in dioxane/water (4:1, 0.5 L) and stirred at 100° C. ON. The OL (water/EtOAc) was washed with brine, dried, concentrated, and purified by FC (heptane to EtOAc) to give tert-butyl 4-(2-fluoro-4-methoxy-carbonyl-6-methyl-phenyl)-3,6-dihydro-2H-pyridine-1-carboxylate (15.5 g). This material and LiOH—H₂O (3.72 g) were stirred ON in MeOH/water (1:1, 0.3 L) and acidified to precipitate 4-(1-tert-butoxycarbonyl-3,6-dihydro-2H-pyridin-4-yl)-3-fluoro-5-methyl-benzoic acid (11.8 g). 5.0 g of this material, HN(CD₃)₂ (1.44 g, HCl salt), and HATU (6.8 g) were stirred ON in DMF/DIPEA (6.4:1, 58 mL), concentrated, and purified by FC (hexane to EtOAc) to give tert-butyl 4-[4-[bis(trideuterio-methyl)carbamoyl]-2-fluoro-6-methyl-phenyl]-3,6-dihydro-2H-pyridine-1-carboxylate (5.1 g). This material was stirred 1 h in HFIP/10M aq. HCl (31:1, 52 mL) and diluted with MTBE to precipitate Int. 1AE12 3-Fluoro-5-methyl-N,N-bis(methyl-d₃)-4-(1,2,3,6-tetrahydropyridin-4-yl)benzamide (3.45 g, HCl salt). Int. 1AE12′ 3-fluoro-5-methyl-N,N-bis(methyl)-4-(1,2,3,6-tetrahydropyridin-4-yl)benzamide was prepared in a similar manner from HN(CH₃)₂.

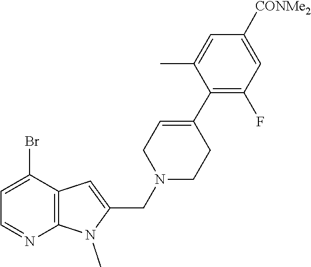

Int. 1J3 (0.50 g) was stirred 1 h in DCM (20 mL) and MsCl (0.8 mL) at 0° C. The OL (water/DCM) was washed with sat. aq. NaHCO₃ and concentrated to give (4-bromo-1-methyl-pyrrolo[2,3-b]pyridin-2-yl)methyl methanesulfonate (0.52 g). 0.5 g of this material, KI (0.13 g), and Int. 1AE12′ (0.51 g) were stirred ON in DMF/DIPEA (14:1, 21.5 mL) at 0° C. to RT. The OL (water/EtOAc) was dried, concentrated, and purified by FC (hexane to EtOAc) to give Int. 1AE13 4-(1-((4-Bromo-1-methyl-1H-pyrrolo[2,3-b]pyridin-2-yl)methyl)-1,2,3,6-tetrahydropyridin-4-yl)-3-fluoro-N,N,5-trimethyl-benzamide (0.52 g).

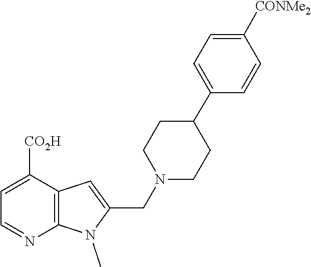

Int. 1ABI (0.15 g), Mo(CO)₆ (87 mg), Pd₂dba₃ (8 mg), and XantPhos (10 mg) were degassed in EtOH (5 mL) and stirred 0.5 h at 120° C. under MW conditions. The mixture was filtered and HPLC-purified give ethyl 2-((4-(4-(dimethylcarbamoyl)phenyl)piperidin-1-yl)methyl)-1-methyl-1H-pyrrolo[2,3-b]pyridine-4-carboxylate (0.25 g). This material and LiOH (69 mg) were stirred in water/EtOH (2:1, 1.5 mL), concentrated, and HPLC-purified to give Int. 1AE15 2-((4-(4-(Dimethylcarbamoyl)phenyl)piperidin-1-yl)methyl)-1-methyl-1H-pyrrolo[2,3-b]pyridine-4-carboxylic acid (0.15 g).

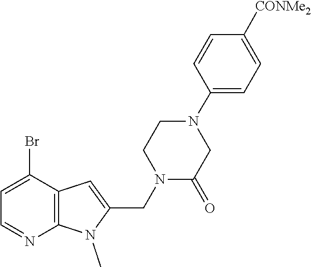

Piperazin-2-one (1.0 g), 4-bromo-N,N-dimethylbenzamide (1.14 g), K₃PO₄ (6.4 g), Pd(OAc)₂ (0.22 g), and RuPhos (0.93 g) were degassed in tert-butyl alcohol (15 mL) and stirred ON at 80-90° C. The mixture was filtered, concentrated, and purified by FC (DCM/MeOH 9:1) to give N,N-dimethyl-4-(3-oxopiperazin-1-yl)benzamide (0.70 g). 0.25 g of this material and NaH (81 mg) were stirred 1 h in THF/DMF (10:1, 5.5 mL) at 0° C. to RT. Int. 1J1 (0.25 g) was added and stirring continued ON at 0° C. to RT. The OL (10% MeOH in DCM/water) was dried, concentrated, and purified by FC (DCM/MeOH 9:1) to give Int. 1AE16 4-(4-((4-Bromo-1-methyl-1H-pyrrolo[2,3-b]pyridin-2-yl)methyl)-3-oxopiperazin-1-yl)-N,N-dimethylbenzamide (0.20 g).

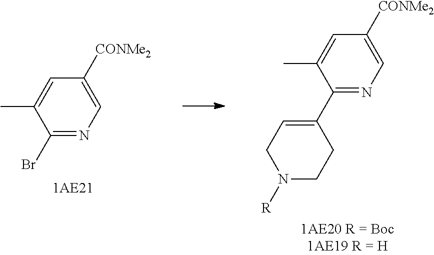

6-Bromo-5-methyl-pyridine-3-carboxylic acid (4.5 g), HATU (9.5 g), and HN(CH₃)₂ (8.4 g, HCl salt) were stirred ON in DMF/DIPEA (2.5:1, 45 mL) at 0° C. to RT. The OL (water/EtOAc) was dried, concentrated and purified by FC (hexane/EtOAc 3:7) to give Int. 1AE21 6-bromo-N,N,5-trimethylnicotinamide (4.2 g). 3.2 g of this material, NaHCO₃ (3.9 g), tert-butyl 4-(4,4,5,5-tetramethyl-1,3,2-dioxaborolan-2-yl)-3,6-dihydro-pyridine-1 (2H)-carboxylate (6.1 g), and PdDPPFCl₂-DCM (0.27 g) were degassed in dioxane/water (3.7:1, 14 mL) and stirred ON at 110° C. The mixture was filtered, concentrated, and purified by FC (hexane/EtOAc 1:4) to give Int. 1AE20 tert-butyl 5-(dimethylcarbamoyl)-3-methyl-3′,6′-dihydro-[2,4′-bipyridine]-1′(2′H)-carboxylate (4.0 g). 0.25 g of this material was stirred ON in DCM/TFA (8.4:1, 5.6 mL) at 0° C. to RT, concentrated, and triturated in Et₂O/pentane to give Int. 1AE19 N,N,5-trimethyl-6-(1,2,3,6-tetrahydro-pyridin-4-yl)pyridine-3-carboxamide (0.28 g, TFA salt).

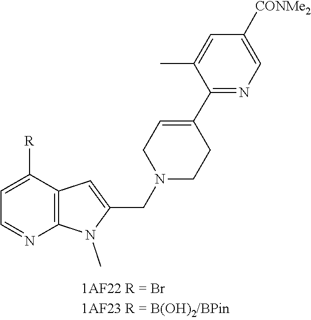

Ints. 1AE19/1J1 (0.5 g/0.5 g) were stirred ON ACN/Et₃N (18:1, 26 mL), concentrated, and HPLC-purified to give Int. 1AE22 6-[1-[(4-bromo-1-methyl-pyrrolo[2,3-b]pyridin-2-yl)methyl]-3,6-dihydro-2H-pyridin-4-yl]-N,N,5-trimethyl-pyridine-3-carboxamide (0.3 g). Int. 1AE22 (3 g), B₂Pin₂ (5.0 g), KOAc (5.0 g), and PdDPPFCl₂-DCM (0.05 g) were degassed in dioxane (25 mL) and stirred ON at 80° C. The OL (water/EtOAc) was dried and concentrated to give Int. 1AE23 (2-((5-(dimethylcarbamoyl)-3-methyl-3′,6′-dihydro-[2,4′-bipyridin]-1′(2′H)-yl)methyl)-1-methyl-1H-pyrrolo[2,3-b]pyridin-4-yl)boronic acid/N,N,3-trimethyl-1′-((1-methyl-4-(4,4,5,5-tetramethyl-1,3,2-dioxaborolan-2-yl)-1H-pyrrolo[2,3-b]pyridin-2-yl)methyl)-1′,2′,3′,6′-tetrahydro-[2,4′-bipyridine]-5-carboxamide (1.0 g).

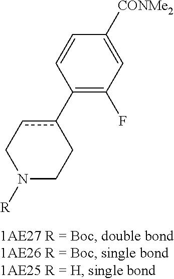

4-Bromo-3-fluoro-N,N-dimethyl-benzamide (1.0 g), tert-butyl 4-(4,4,5,5-tetramethyl-1,3,2-dioxaborolan-2-yl)-3,6-dihydro-2H-pyridine-1-carboxylate (1.51 g), Na₂CO₃ (1.72 g), and PdDPPFCl₂-DCM (0.17 g) were degassed in dioxane/water (4:1, 10 mL) and stirred at 100° C. ON. The OL (water/EtOAc) was washed with brine, dried, concentrated, and purified by FC (hexane to EtOAc) to give Int. 1AE27 tert-butyl 4-[4-(dimethyl-carbamoyl)-2-fluoro-phenyl]-3,6-dihydro-2H-pyridine-1-carboxylate (1.2 g). This material was hydrogenated ON using 10% Pd/C (40 mg) in MeOH (10 mL) under an atmosphere of H₂ in a sealed tube, filtered, and concentrated to give Int. 1AE26 tert-butyl 4-[4-(dimethylcarba-moyl)-2-fluoro-phenyl]piperidine-1-carboxylate (0.60 g) that was stirred in 4M HCl in dioxane (10 mL) ON, concentrated, and HPLC-purified to give Int. 1AE25 3-fluoro-N,N-dimethyl-4-(piperidin-4-yl)benzamide (63 mg).

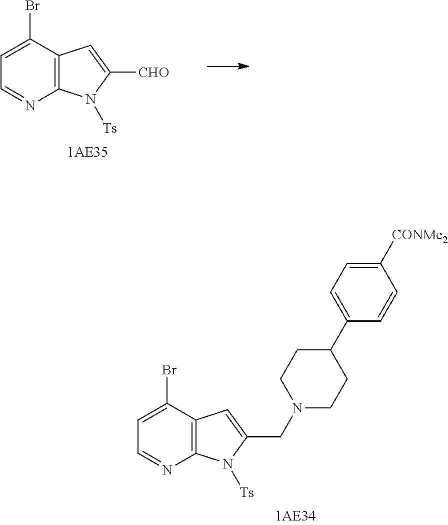

4-Bromo-1-tosyl-1H-pyrrolo[2,3-b]pyridine (1.0 g) was stirred 1 h in 0.6 M LDA in THF (14 mL) at −78° C. DMF (1.3 mL) was added and stirring continued 2 h at −78° C. The OL (sat. aq. NH₄Cl/EtOAc) was dried, concentrated, and purified by FC (hexane/EtOAc 9:1) to give Int. 1AE35 4-bromo-1-(p-tolyl-sulfonyl)pyrrolo[2,3-b]pyridine-2-carbaldehyde (0.6 g). Int. 1A35 (1.5 g) and N,N-dimethyl-4-(piperidin-4-yl)benzamide (1.6 g, HCl salt) were stirred ON in DCM/DIPEA (5:1, 24 mL). STAB (2.5 g) was added and stirring continued 2 h at 0° C. to RT. The OL (DCM/water) was dried, concentrated, and purified by FC (DCM/MeOH 9:1) to give Int. 1AE34 4-[1-[[4-bromo-1-(p-tolylsulfonyl)-pyrrolo[2,3-b]pyridin-2-yl]methyl]-4-piperidyl]-N,N-dimethyl-benzamide (1.0 g).

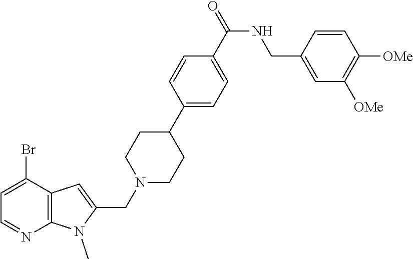 [SMILES: COc1ccc(CNC(=O)c2ccc(C3CCN(Cc4cc5c(Br)ccnc5n4C)CC3)cc2)cc1OC]

Int. 1AC4 (0.10 g), HATU (164 mg), and (3,4-dimethoxybenzyl)(methyl)-amine (58 mg) were stirred 3 h in DMF/DIPEA (25:1, 3.1 mL). The OL (sat. aq. NaHCO₃/DCM) was dried, concentrated, and purified by FC (DCM to DCM/MeOH 93:7) to give Int. 1AE36 4-(1-((4-bromo-1-methyl-1H-pyrrolo-[2,3-b]pyridin-2-yl)methyl)piperidin-4-yl)-N-(3,4-dimethoxybenzyl)-N-methylbenzamide (0.12 g).

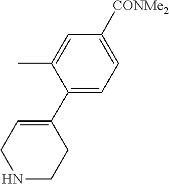

tert-Butyl 4-[4-(dimethylcarbamoyl)-2-methyl-phenyl]-3,6-dihydro-2H-pyridine-1-carboxylate (4.0 g) was stirred 0.5 h in 4M HCl in dioxane (40 mL), concentrated, and triturated in Et₂O to give Int. 1AE38 N,N,3-trimethyl-4-(1,2,3,6-tetrahydropyridin-4-yl)benzamide (1.06 g, HCl salt).

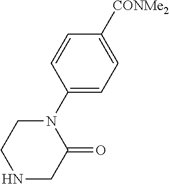

tert-Butyl 3-oxopiperazine-1-carboxylate (0.50 g), 4-bromo-N,N-dimethylbenzamide (0.68 g), K₃PO₄ (1.06 g), CuI (71 mg), DMDCH (36 mg), were degassed in dioxane (10 mL) and was stirred at 100° C. ON. The OL (water/EtOAc) was dried, concentrated, and purified by FC (EtOAc/MeOH 1:0 to 9:1) to give tert-butyl 4-(4-(dimethylcarbamoyl)-phenyl)-3-oxopiperazine-1-carboxylate (0.80 g). 0.23 g of this material was stirred 1 h in DCM/TFA (1:1, 6 mL) and concentrated to give Int. 1AE39 N,N-Dimethyl-4-(2-oxo-piperazin-1-yl)benzamide (0.24 g, TFA salt).

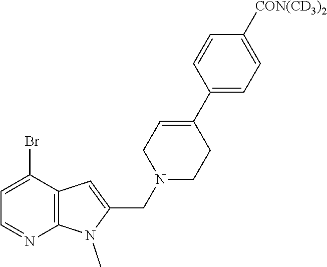

Int. 1J1/1AE7′ (0.33 g/0.30 g), and KI (21 mg) were stirred ON in DMF/DIPEA (7.3:1, 9 mL) at 0° C. to RT. The OL (water/EtOAc) was dried, concentrated, and purified by FC (hexane to EtOAc) to give Int. 1AF1 4-(1-((4-Bromo-1-methyl-1H-pyrrolo[2,3-b]pyridin-2-yl)methyl)-1,2,3,6-tetrahydropyridin-4-yl)-N,N-bis(methyl-d₃)benzamide (0.20 g).

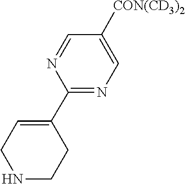

Methyl 2-bromopyrimidine-5-carboxylate (10.0 g), tert-butyl 4-(4,4,5,5-tetramethyl-1,3,2-dioxaborolan-2-yl)-3,6-dihydropyridine-1 (2H)-carboxylate (15.6 g), K₂CO₃ (25.5 g), and PdDPPFCl₂-DCM (1.88 g) were degassed in dioxane/water (4:1, 0.25 L) and stirred ON at 100° C. The OL (water/EtOAc) was washed with brine, dried, concentrated, and purified by FC (hexane to EtOAc) to give methyl 2-(1-tert-butoxycarbonyl-3,6-dihydro-2H-pyridin-4-yl)pyrimidine-5-carboxylate (14.7 g). This material and LiOH—H₂O (2.15 g) were stirred ON in MeOH/water (1:1, 200 mL) and acidified to precipitate 2-(1-tert-butoxy-carbonyl-3,6-dihydro-2H-pyridin-4-yl)pyrimidine-5-carboxylic acid (8.0 g). 4.0 g of this material, HN(CD₃)₂ (1.21 g, HCl salt), and HATU (5.27 g) were stirred in DMF/DIPEA (3:1, 27 mL), concentrated, and purified by FC (hexane to EtOAc) to give tert-butyl 4-[5-[bis(tri-deuteriomethyl)carbamoyl]pyrimidin-2-yl]-3,6-dihydro-2H-pyridine-1-carboxylate (2.20 g). This material was stirred in 1 h HFIP/10M aq. HCl (30:1, 23 mL), concentrated, and triturated in MTBE to give Int. 1AF2 N,N-bis(methyl-d₃)-2-(1,2,3,6-tetrahydropyridin-4-yl)pyrimidine-5-carboxamide (1.40 g, HCl salt).

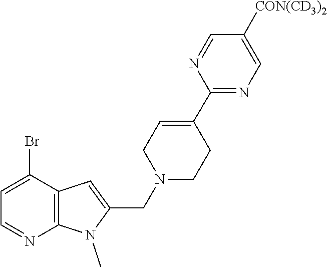

Ints. 1AF2/1J1 (0.5 g/0.5 g) were stirred in ACN/Et₃N (19:1, 26 mL), concentrated, and HPLC-purified to give Int. 1AF3 2-[1-[(4-Bromo-1-methyl-pyrrolo[2,3-b]pyridin-2-yl)methyl]-3,6-dihydro-2H-pyridin-4-yl]-N,N-bis(trideuteriomethyl)pyrimidine-5-carboxamide (0.3 g).

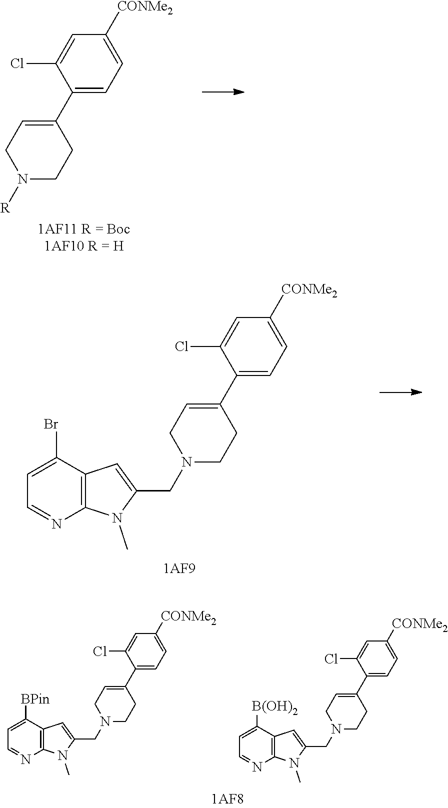

4-Bromo-3-fluoro-N,N-dimethyl-benzamide (1 g), tert-butyl 4-(4,4,5,5-tetramethyl-1,3,2-dioxaborolan-2-yl)-3,6-dihydropyridine-1 (2H)-carboxylate (1.5 g), Na₂CO₃ (1.7 g), PdPDDFCl₂-DCM (0.17 g) were deassed in dioxane/water (4:1, 10 mL) and stirred ON at 100° C. The OL (water/EtOAc) was washed with brine, dried, concentrated, and purified by FC (hexane to EtOAc) to give Int. 1AF11 tert-butyl 4-[4-(dimethylcarbamoyl)-2-fluoro-phenyl]-3,6-dihydro-2H-pyridine-1-carboxylate (1.2 g). Int. 1AF11 (0.6 g) was stirred ON in 4M HCl in dioxane (10 mL). The residue after concentration was HPLC-purified to give Int. 1AF10 3-fluoro-N,N-dimethyl-4-(1,2,3,6-tetrahydropyridin-4-yl)benzamide (0.22 g). Ints. 1AF10/1J1 (0.5 g/0.5 g) were stirred ON in ACN/Et₃N (19:1, 26 mL), concentrated, and HPLC-purified to give Int. 1AF9 4-[1-[(4-bromo-1-methyl-pyrrolo[2,3-b]pyridin-2-yl)-methyl]-3,6-dihydro-2H-pyridin-4-yl]-N,N,3,5-tetramethyl-benzamide (0.3 g). Int. 1AF9 (3 g), B₂pin₂ (5 g), KOAc (5 g), and PdDPPFCl₂-DCM (50 mg) were degassed in dioxane and stirred ON at 80° C. The OL (EtOAc/water) was dried and concentrated to give Int. 1AF8 3-chloro-N,N-dimethyl-4-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)benzamide/(2-((4-(2-chloro-4-(dimethylcarbamoyl)phenyl)-3,6-dihydropyridin-1 (2H)-yl)methyl)-1-methyl-1H-pyrrolo[2,3-b]pyridin-4-yl)boronic acid (1 g).

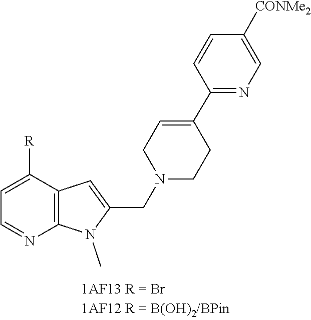

Ints. 1S5/1J1 (0.5 g/0.5 g, as the HCl salt) were stirred ON in ACN/Et₃N (19:1, 26 mL), concentrated, and HPLC-purified to give Int. 1AF13 6-[1-[(4-bromo-1-methyl-pyrrolo-[2,3-b]pyridin-2-yl)methyl]-3,6-dihydro-2H-pyridin-4-yl]-N,N-dimethyl-pyridine-3-carboxamide (0.3 g). Int. 1AF13 (3.0 g), KOAc (5.0 g), B₂Pin₂ (5.0 g), and PdDPPFCl₂-DCM (50 mg) were degassed in dioxane (25 mL) and stirred at 80° C. ON. The OL (water/EtOAc) was concentrated to give Example 1AF12 N,N-Dimethyl-1′-((1-methyl-4-(4,4,5,5-tetramethyl-1,3,2-dioxaborolan-2-yl)-1H-pyrrolo[2,3-b]pyridin-2-yl)methyl)-1′,2′,3′,6′-tetrahydro-[2,4′-bipyridine]-5-carboxamide/(2-((5-(dimethylcarbamoyl)-3′,6′-dihydro-[2,4′-bipyridin]-1′(2′H)-yl)methyl)-1-methyl-1H-pyrrolo[2,3-b]pyridin-4-yl)boronic acid (1.0 g).

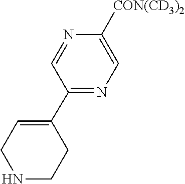

Methyl 5-bromopyrazine-2-carboxylate (11.0 g), tert-butyl 4-(4,4,5,5-tetramethyl-1,3,2-dioxaborolan-2-yl)-3,6-dihydropyridine-1 (2H)-carboxylate (17.2 g), K₂CO₃ (28.0 g), PdPDDFCl₂-DCM (2.07 g) were deassed in dioxane/water (5:1, 250 mL) and stirred ON at 100° C. The aq. layer (water/EtOAc) was acidified to precipitate 5-(1-tert-butoxy-carbonyl-3,6-dihydro-2H-pyridin-4-yl)pyrazine-2-carboxylic acid (12.0 g). 4.5 g of this material, HN(CD₃)₂ (1.40 g, HCl salt), and HATU (6.20 g) were stirred ON in DMF/DIPEA (2.6:1, 28 mL), concentrated, and purified by FC (hexane to EtOAc) to give tert-butyl 4-[5-[bis(trideuteriomethyl)-carbamoyl]pyrazin-2-yl]-3,6-dihydro-2H-pyridine-1-carboxylate (2.0 g). This material was stirred in 1 h HFIP/10M aq. HCl (29:1, 21 mL), concentrated, and triturated in MTBE to give Int. 1AF14 5-(1,2,3,6-Tetrahydropyridin-4-yl)-N,N-bis(trideuteriomethyl)pyrazine-2-carboxamide (0.88 g, HCl salt).

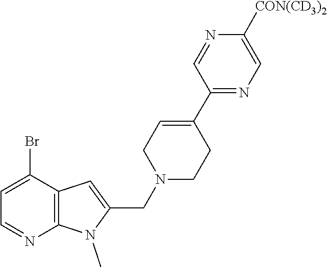

Ints. 1AF14/1J1 (0.5 g/0.5 g), and Et₃N (1.2 mL) were stirred ON in ACN (25 mL), concentrated, and HPLC-purified to give Int. 1AF15 5-(1-((4-Bromo-1-methyl-1H-pyrrolo[2,3-b]pyridin-2-yl)methyl)-1,2,3,6-tetrahydropyridin-4-yl)-N,N-bis(methyl-d₃)pyrazine-2-carboxamide (0.3 g).

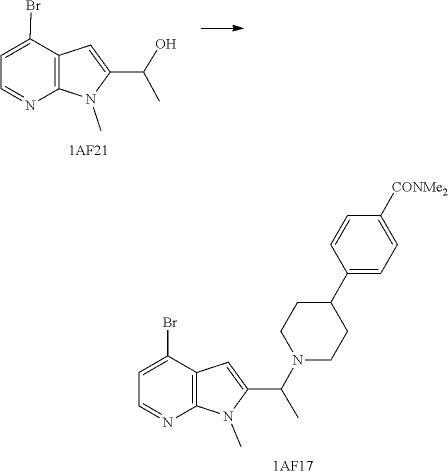

1.0M MeMgBr in THF (185 mL) was added to a solution of Int. 1AB8 (29.5 g) in THF (1.2 L) at 0° C. and stirring continued 3 h. The OL (sat. aq. NH₄Cl/EtOAc) was washed with brine, dried, concentrated and purified by FC (pentane/EtOAc 3:1) to give Int. 1AF21 1-(4-bromo-1-methyl-1H-pyrrolo[2,3-b]pyridin-2-yl)ethan-1-ol (25.5 g). 0.30 g of this material was stirred in toluene/SOCl₂ (12.5:1, 10.8 mL) at 0° C. before stirring 0.5 h at 110° C. The mixture was concentrated. The residue, Cs₂CO₃ (1.15 g), KI (0.98 g), and N,N-dimethyl-4-(4-piperidyl)-benzamide (0.41 g) were stirred ON in ACN (10 mL) at 0° C. to RT. The OL (water/EtOAc) was washed with brine, dried, concentrated, and purified by FC (DCM/MeOH 95:5) to give Int. 1AF17 4-(1-(1-(4-bromo-1-methyl-1H-pyrrolo[2,3-b]pyridin-2-yl)ethyl)-piperidin-4-yl)-N,N-dimethylbenzamide (0.28 g).

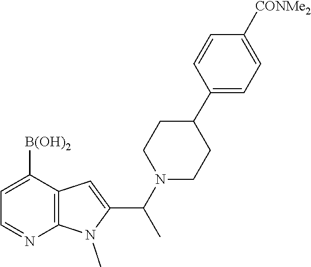

Int. 1AF17 (0.70 g), B₂Pin₂ (0.51 g), KOAc (0.4 g), and Pd(PPh₃)₂Cl₂ (0.1 g) were degassed in THF (50 mL) and stirred ON at 100° C., filtered, concentrated, and triturated in pentane to give Int. 1AF24 (2-(1-(4-(4-(dimethyl-carbamoyl)phenyl)piperidin-1-yl)ethyl)-1-methyl-1H-pyrrolo[2,3-b]pyridin-4-yl)boronic acid (0.85 g).

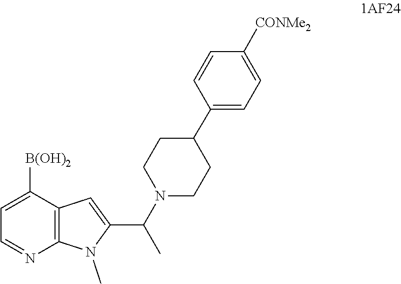

Int. 1AF17 (0.70 g), B₂Pin₂ (0.51 g), KOAc (0.4 g), and Pd(PPh₃)₂Cl₂ (0.1 g) were degassed in THF (50 mL) and stirred ON at 100° C., filtered, concentrated, and triturated in pentane to give Int. 1AF24 (2-(1-(4-(4-(dimethyl-carbamoyl)phenyl)piperidin-1-yl)ethyl)-1-methyl-1H-pyrrolo[2,3-b]pyridin-4-yl)boronic acid (0.85 g).

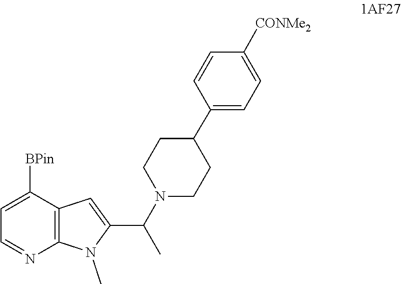

Int. 1AF17 (0.70 g), B₂Pin₂ (38 mg), KOAc (45 mg), and PdDPPFCl₂-DCM (25 mg) were degassed in dioxane (10 mL) and stirred ON at 110° C. The mixture was filtered, concentrated, and purified by FC (pentane/EtOAc 95:5) to give Int. 1AF27 N,N-dimethyl-4-(1-(1-(1-methyl-4-(4,4,5,5-tetramethyl-1,3,2-dioxaborolan-2-yl)-1H-pyrrolo-[2,3-b]pyridin-2-yl)ethyl)piperidin-4-yl)benzamide (0.85 g).

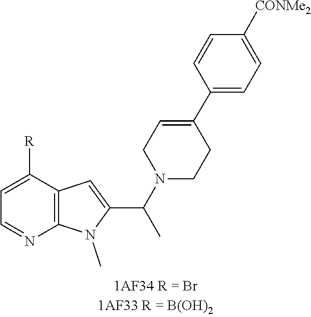

1-(4-Bromo-1-methyl-1H-pyrrolo[2,3-b]pyridin-2-yl)ethan-1-ol (0.20 g) was stirred 1.5 h in toluene/SOCl₂ (29:1, 5.2 mL) at 0° C. to 110° C. and concentrated. The residue, KI (10 mg), and Int. 1AF5 (0.23 g) were stirred ON in DMF/DIPEA (2.5:1, 2.8 mL) at 0° C. to RT. The OL (water/EtOAc) was washed with brine, dried, concentrated, and purified by FC (hexane/EtOAc 9:1 to 85:15) to give Int. 1AF34 4-[1-[1-(4-bromo-1-methyl-pyrrolo[2,3-b]pyridin-2-yl)ethyl]-3,6-dihydro-2H-pyridin-4-yl]-N,N-dimethyl-benzamide (80 mg). Int. 1AF34 (0.32 g), B₂Pin₂ (24 mg), KOAc (25 mg), and PdCl₂(PPh₃)₂ (48 mg) were degassed in THF (5 mL) and stirred ON at 90° C. The mixture was filtered, concentrated, and purified by FC (pentane/EtOAc 95:5) to give Int. 1AF33 (2-(1-(4-(4-(dimethylcarbamoyl)-phenyl)-3,6-dihydropyridin-1 (2H)-yl)ethyl)-1-methyl-1H-pyrrolo[2,3-b]pyridin-4-yl)-boronic acid (0.50 g).

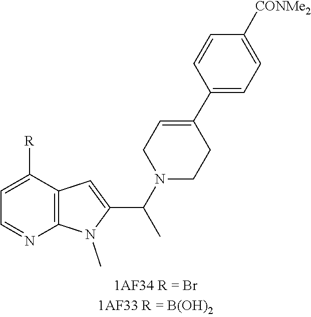

1-(4-Bromo-1-methyl-1H-pyrrolo[2,3-b]pyridin-2-yl)ethan-1-ol (0.20 g) was stirred 1.5 h in toluene/SOCl₂ (29:1, 5.2 mL) at 0° C. to 110° C. and concentrated. The residue, KI (10 mg), and Int. 1AF5 (0.23 g) were stirred ON in DMF/DIPEA (2.5:1, 2.8 mL) at 0° C. to RT. The OL (water/EtOAc) was washed with brine, dried, concentrated, and purified by FC (hexane/EtOAc 9:1 to 85:15) to give Int. 1AF34 4-[1-[1-(4-bromo-1-methyl-pyrrolo-[2,3-b]pyridin-2-yl)ethyl]-3,6-dihydro-2H-pyridin-4-yl]-N,N-dimethyl-benzamide (80 mg). Int. 1AF34 (0.32 g), B₂Pin₂ (24 mg), KOAc (25 mg), and PdCl₂(PPh₃)₂ (48 mg) were degassed in THF (5 mL) and stirred ON at 90° C. The mixture was filtered, concentrated, and purified by FC (pentane/EtOAc 95:5) to give Int. 1AF33 (2-(1-(4-(4-(dimethyl-carbamoyl)-phenyl)-3,6-dihydropyridin-1 (2H)-yl)ethyl)-1-methyl-1H-pyrrolo[2,3-b]pyridin-4-yl)-boronic acid (0.50 g).

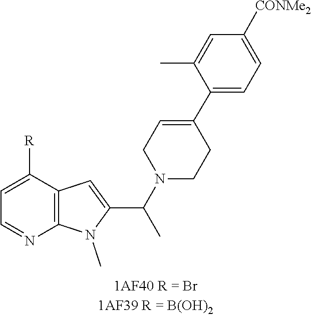

Int. 1AF40 4-[1-[1-(4-bromo-1-methyl-pyrrolo[2,3-b]pyridin-2-yl)ethyl]-3,6-dihydro-2H-pyridin-4-yl]-N,N,3-trimethyl-benzamide (0.15 g) was prepared similarly to Int. 1AF17 from Int. 1AF21 (0.20 g) and Int. 1AE38 (0.37 g, TFA salt). Int. 1AF39 (2-(1-(4-(4-(dimethylcarbamoyl)-2-methylphenyl)-3,6-dihydropyridin-1 (2H)-yl)-ethyl)-1-methyl-1H-pyrrolo[2,3-b]pyridin-4-yl)boronic acid (40 mg) was prepared similarly to Int. 1AF33 from Int. 1AF40 (50 mg).

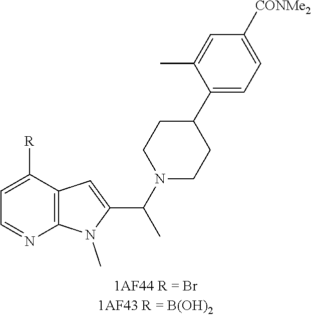

Int. 1AF44 4-[1-[1-(4-bromo-1-methyl-pyrrolo[2,3-b]pyridin-2-yl)ethyl]-4-piperidyl]-N,N,3-trimethyl-benzamide (0.15 g) was prepared similarly to Int. 1AF17 from Ints. 1AF24/1AE38 (0.20 g/0.42 g, TFA salt). Int. 1AF43 (2-(1-(4-(4-(dimethylcarbamoyl)-2-methylphenyl)-3,6-dihydropyridin-1 (2H)-yl)ethyl)-1-methyl-1H-pyrrolo[2,3-b]-pyridin-4-yl)boronic acid (0.60 g) was prepared similarly to Int. 1AF39 from Int. 1AF44 (0.50 g).

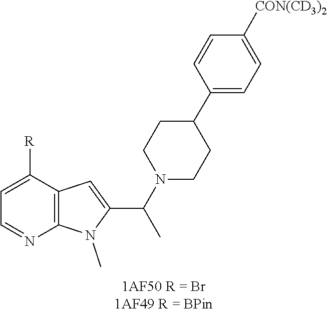

Int. 1AF50 4-[1-[1-(4-bromo-1-methyl-pyrrolo[2,3-b]pyridin-2-yl)ethyl]-4-piperidyl]-N,N-bis(-methyl)benzamid (0.65 g) was prepared similarly to Int. 1AF17 from Ints. 1AF21/1D7 (0.35 g/0.53 g, TFA salt). Int. 1AF49 [2-[1-[4-[4-[bis(trideuterio-methyl)carbamoyl]phenyl]-1-piperidyl]ethyl]-1-methyl-pyrrolo[2,3-b]-pyridin-4-yl]-boronic acid (0.41 g) was prepared similarly to Int. 1AF25 from Int. 1AF50 (0.32 g) and B₂Pin₂ (0.34 g).

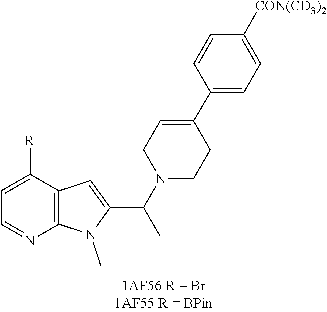

Int. 1AF56 4-(1-(1-(4-bromo-1-methyl-1H-pyrrolo[2,3-b]pyridin-2-yl)ethyl)-1,2,3,6-tetrahydropyridin-4-yl)-N,N-bis(methyl-d₃)benzamide (0.60 g) was prepared similarly to Int. 1AF34 from Int. 1AF21 (0.50 g) and Int. 1AE7′ (0.69 g). Int. 1AF55 [2-[1-[4-[4-[bis(trideuteriomethyl)carbamoyl]phenyl]-3,6-dihydro-2H-pyridin-1-yl]ethyl]-1-methyl-pyrrolo[2,3-b]pyridin-4-yl]boronic acid (0.55 g) was prepared similarly to Int. 1AF39 from Int. 1AF56 (0.50 g).

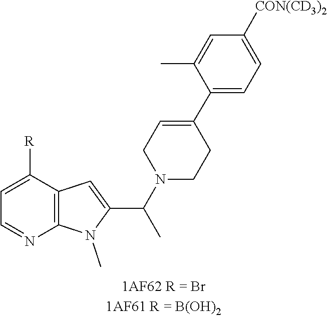

Int. 1AF62 4-(1-(1-(4-bromo-1-methyl-1H-pyrrolo[2,3-b]pyridin-2-yl)ethyl)-1,2,3,6-tetrahydro-pyridin-4-yl)-3-methyl-N,N-bis(methyl-d₃)benzamide (0.46 g) was prepared similarly to Int. 1AF17 from Ints. 1AF24/1S20 (0.50 g/0.70 g). Int. 1AF61 (2-(1-(4-(4-(bis(methyl-d₃)carbamoyl)-2-methylphenyl)-3,6-dihydropyridin-1 (2H)-yl)ethyl)-1-methyl-1H-pyrrolo[2,3-b]pyridin-4-yl)boronic acid (0.75 g) was prepared similarly to Int. 1AF55 from B₂Pin₂ (0.52 g) and Int. 1AF62 (0.5 g).

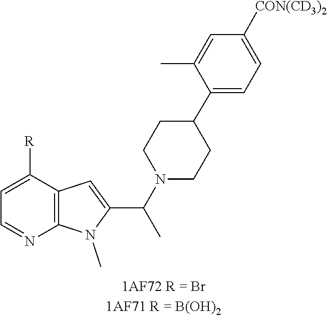

Int. 1AF72 4-(1-(1-(4-bromo-1-methyl-1H-pyrrolo[2,3-b]pyridin-2-yl)ethyl)piperidin-4-yl)-3-methyl-N,N-bis-(methyl-d₃)benzamide (90 mg) was prepared similarly to Int. 1AF17 from Ints. 1AF24/1S22 (0.10 g/0.19 g, TFA salt). Int. 1AF71 (2-(1-(4-(4-(bis(methyl-d₃)carbamoyl)-2-methylphenyl)piperidin-1-yl)ethyl)-1-methyl-1H-pyrrolo[2,3-b]pyridin-4-yl)boronic acid (0.10 g) was prepared similarly to Int.1AF55 from Int 1AF72 (90 mg) and B₂Pin₂ (0.12 g).

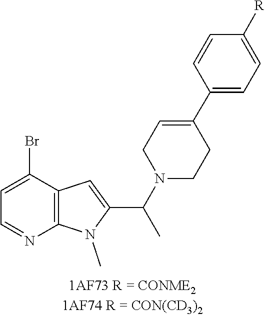

Int. 1AF21 (1.20 g) was stirred 1 h in toluene/SOCl₂ (5.9:1, 23.5 mL) at 0° C. to 110° C. and concentrated. The residue, Cs₂CO₃ (4.60 g), KI (0.39 g), and Int. 1AE7 (1.30 g) were stirred ON in ACN (30 mL) at 0° C. to RT. The OL (water/EtOAc) was washed with brine, dried, concentrated, and purified by FC (hexane/EtOAc 9:1 to 85:15) to give Int. 1AF73 4-[1-[1-(4-bromo-1-methyl-pyrrolo[2,3-b]pyridin-2-yl)ethyl]-3,6-dihydro-2H-pyridin-4-yl]-N,N-dimethyl-benzamide (1.0 g). Int. 1AF74 4-(1-(1-(4-bromo-1-methyl-1H-pyrrolo[2,3-b]pyridin-2-yl)ethyl)-1,2,3,6-tetrahydropyridin-4-yl)-N,N-bis(methyl-d₃)benzamide (0.60 g) was prepared similarly from Ints. 1AE7′/1AF21 (0.69 g/0.50 g).

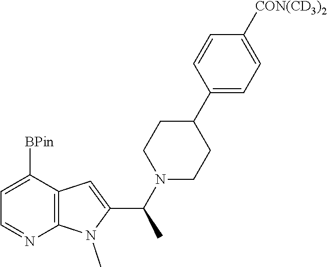

Int. 1AF76 (1.50 g), B₂Pin₂ (1.60 g), KOAc (0.93 g), and PdPDDFCl₂-DCM (0.26 g) were degassed in dioxane (30 mL) and stirred 6 h at 90° C., filtered, concentrated, and triturated in Et₂O to give Int. 1AF75 (S)—N,N-bis(methyl-d₃)-4-(1-(1-(1-methyl-4-(4,4,5,5-tetramethyl-1,3,2-dioxaborolan-2-yl)-1H-pyrrolo[2,3-b]pyridin-2-yl)ethyl)-piperidin-4-yl)benzamide (1.30 g).

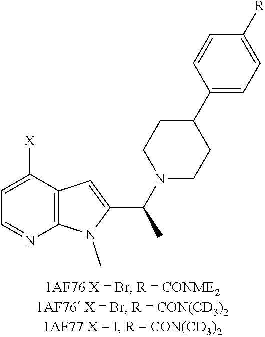

Int. 2C36 (0.35 g), HATU (0.3 g), and HN(CD₃)₂ (77 mg, HCl salt) were stirred ON in DMF/DIPEA (14.7:1, 10.7 mL) at 0° C. to RT and diluted with water to precipitate Int. 1AF76 (0.30 g). Int. 1AF76′ (3.5 g) was prepared similarly from Int. 2C36 (4.5 g). Int. 1AF76′ (0.10 g), CuI (20 mg), NaI (63 mg), and DMDCH (30 mg) were degassed in dioxane (5 mL) and stirred at 110° C. ON and filtered. The OL (DCM/aq. NH₃) was dried, concentrated, and purified by FC (DCM/MeOH 97:3) to give Int. 1AF77 (S)-4-(1-(1-(4-iodo-1-methyl-1H-pyrrolo[2,3-b]pyridin-2-yl)ethyl)piperidin-4-yl)-N,N-bis(methyl-d₃)-benzamide (50 mg). The absolute configuration of 1AF76 (S)-4-(1-(1-(4-bromo-1-methyl-1H-pyrrolo-[2,3-b]pyridin-2-yl)-ethyl)piperidin-4-yl)-N,N-dimethylbenzamide and 1AF76′ (S)-4-(1-(1-(4-bromo-1-methyl-1H-pyrrolo-[2,3-b]pyridin-2-yl)ethyl)-piperidin-4-yl)-N,N-bis(methyl-d₃)benzamide was determined to S by VCD.

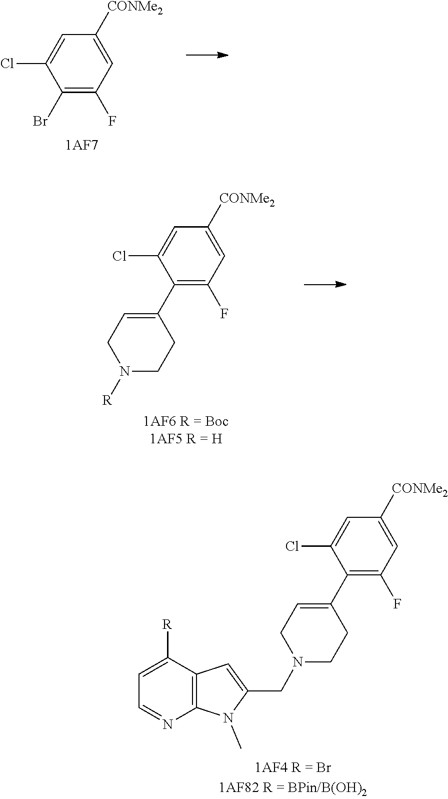

CDI (7.04 g) was added to a solution of 4-bromo-3-fluoro-5-methyl-benzoyl chloride (11.0 g) in DCM (50 mL) at 0° C. and stirring continued 0.5 h. HN(CH₃)₂ (7.1 g, HCl salt) was added before refluxing 5 h. The mixture was washed with water, dried, and concentrated to give Int. 1AF7 4-bromo-3-fluoro-N,N,5-trimethyl-benzamide (12.4 g). This material, tert-butyl 4-(4,4,5,5-tetramethyl-1,3,2-dioxa-borolan-2-yl)-3,6-dihydro-2H-pyridine-1-carboxylate (15.0 g), K₂CO₃ (18.3 g), and PdDPPFCl₂-DCM (0.73 g) were degassed in dioxane (100 mL) and stirred 48 h at 90° C. The OL (water/MTBE) was dried and concentrated to give Int. 1AF6 tert-butyl 4-(2-chloro-4-(dimethylcarbamoyl)-6-fluorophenyl)-3,6-dihydropyridine-1 (2H)-carboxylate (11.0 g). Int. 1AF6 (0.6 g) was stirred 0.5 h in THF/4M HCl in dioxane (1:1, 2 mL). The OL (water/EtOAc) was dried and concentrated to give Int. 1AF5 3-chloro-5-fluoro-N,N-dimethyl-4-(1,2,3,6-tetrahydro-pyridin-4-yl)benzamide (0.25 g, HCl salt). This material, K₂CO₃ (0.37 g), and Int. 1J1 (0.23 g) were stirred in DMF (5 mL) ON. The OL (water/MTBE) was layer was dried and concentrated to give Int. 1AF4 4-[1-[(4-bromo-1-methyl-pyrrolo[2,3-b]pyridin-2-yl)-methyl]-3,6-dihydro-2H-pyridin-4-yl]-3-chloro-5-fluoro-N,N-dimethyl-benzamide (0.17 g). This material, B₂Pin₂ (0.17 g), KOAc (0.10 g), and PdDPPFCl₂-DCM (6 mg) were degassed in dioxane (5 mL) and stirred 48 h at 100° C. The OL (water/MTBE) was layer was dried and concentrated to give Int. 1AF82 3-Chloro-5-fluoro-N,N-dimethyl-4-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)benzamide/(2-((4-(2-chloro-4-(dimethyl-carbamoyl)-6-fluorophenyl)-3,6-dihydropyridin-1 (2H)-yl)methyl)-1-methyl-1H-pyrrolo[2,3-b]pyridin-4-yl)-boronic acid (0.19 g).

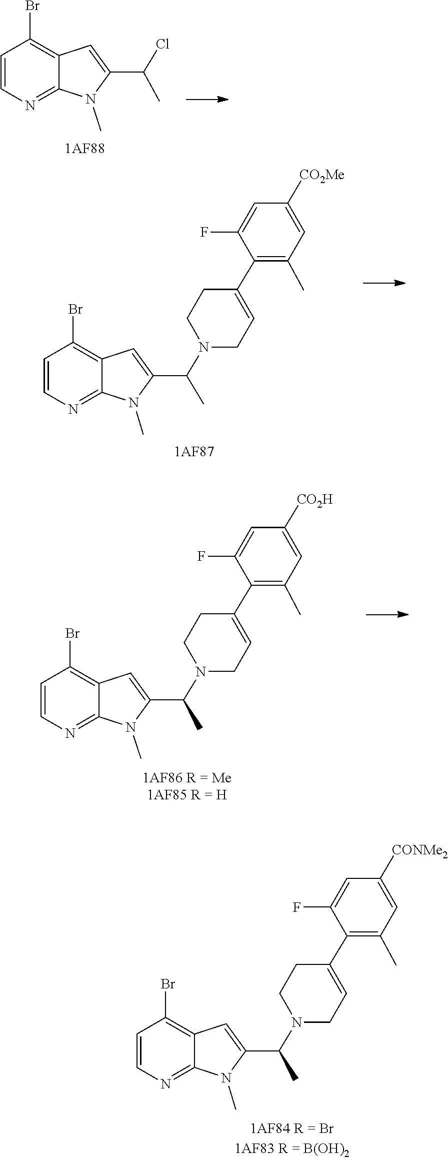

Int. 1AF21 (8 g) was stirred 0.5 h in toluene/SOCl₂ (4:1, 123 mL) at 0° C. to 80° C. and concentrated to give Int. 1AF88 (7.5 g). Ints. 1AF88/1AF90 (7.5 g, 7.8 g, HCl salt), Cs₂CO₃ (31 g), and KI (23 g) were stirred ON in ACN (100 mL) at 0° C. to RT. The mixture was filtered, concentrated, and purified by FC (hexane/EtOAc 41:9) to give Int. 1AF87 methyl 4-[1-[1-(4-bromo-1-methyl-pyrrolo[2,3-b]pyridin-2-yl)ethyl]-3,6-dihydro-2H-pyridin-4-yl]-3-fluoro-5-methyl-benzoate (3.9 g). Int. 1AF87 (0.2 g) was resolved by SFC using a Chiralpak AD-H 250×21 mm 5 μm column operated at 30° C. and an eluent of 70% CO₂ and 30% MeOH (70 g/min) and a back pressure of 100 bar to give Int. 1AF86 methyl (S)-4-(1-(1-(4-bromo-1-methyl-1H-pyrrolo[2,3-b]pyridin-2-yl)ethyl)-1,2,3,6-tetrahydropyridin-4-yl)-3-fluoro-5-methylbenzoate (70 mg, second peak). The absolute configuration was determined by VCD for Int. 1AF86. Int. 1AF86 (2.5 g) and LiOH (1.1 g) were stirred 1 h in THF/MeOH/water (2:1:1, 20 mL) at 0° C. to RT. The mixture was concentrated and triturated in aq. citric acid to precipitate Int. 1AF85 4-[1-[(1S)-1-(4-bromo-1-methyl-pyrrolo[2,3-b]pyridin-2-yl)ethyl]-3,6-dihydro-2H-pyridin-4-yl]-3-fluoro-5-methyl-benzoic acid (2.3 g). 4-[1-[(1S)-1-(4-bromo-1-methyl-pyrrolo[2,3-b]pyridin-2-yl)ethyl]-3,6-dihydro-2H-pyridin-4-yl]-3-fluoro-5-methyl-benzoic acid (1.8 g), HATU (2.2 g), and HNMe₂ (0.93 g, HCl salt) were stirred ON in DMF/DIPEA (6.1:1, 23.3 mL) at 0° C. to RT. The mixture was diluted with water to precipitate Int. 1AF84 4-[1-[(1S)-1-(4-bromo-1-methyl-pyrrolo[2,3-b]pyridin-2-yl)ethyl]-3,6-dihydro-2H-pyridin-4-yl]-3-fluoro-N,N,5-trimethyl-benzamide (1.5 g). Int. 1AF84′ (S)-4-(1-(1-(4-bromo-1-methyl-1H-pyrrolo[2,3-b]pyridin-2-yl)ethyl)-1,2,3,6-tetrahydropyridin-4-yl)-3-fluoro-5-methyl-N,N-bis(methyl-d₃)benzamide was prepared similarly using the HCl salt of HN(CD₃)₂. Int. 1AF84 (1.1 g), KOAc (0.86 g), B₂Pin₂ (1.1 g), and PdDPPFCl₂-DCM (0.18 g) stirred 1 h in dioxane (10 mL) at 110° C. under MW conditions. The mixture was filtered, concentrated, and triturated in pentane to give Int. 1AF83 [2-[(1S)-1-[4-[4-(dimethylcarbamoyl)-2-fluoro-6-methyl-phenyl]-3,6-dihydro-2H-pyridin-1-yl]ethyl]-1-methyl-pyrrolo[2,3-b]pyridin-4-yl]boronic acid (1.3 g).

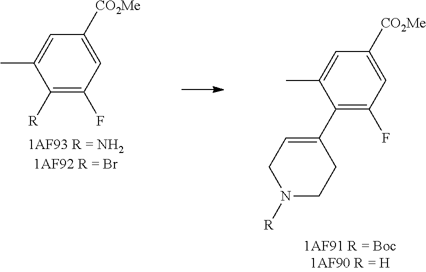

2-Fluoro-4-iodo-6-methyl-aniline (20 g) and PdDPPFCl₂-DCM were stirred ON in MeOH/Et₃N (23:1, 522 mL) under an atmosphere of CO (200 psi) at 80° C. The mixture was filtered. The OL (EtOAc/10% aq. EDTA) was dried and concentrated to afford Int. 1AF93 methyl 4-amino-3-fluoro-5-methyl-benzoate (14 g). Int. 1AF93 (25 g), tert-butyl nitrite (21.1 g), and CuBr₂ (152 g) were stirred ON in ACN (500 mL) at 0° C. to 80° C. The mixture was cooled to 0° C., diluted with sat. aq. NaHCO₃, and filtered. The OL (EtOAc/10% aq. NH₃) was concentrated and purified by FC (hexane/EtOAc 9:1) to give Int. 1AF92 methyl 4-bromo-3-fluoro-5-methyl-benzoate (21 g). Int. 1AF92 (1.0 g), tert-butyl 4-(4,4,5,5-tetramethyl-1,3,2-dioxaborolan-2-yl)-3,6-dihydropyridine-1 (2H)-carboxylate (1.9 g), NaHCO₃ (1.29 g), and PdDPPFCl₂-DCM (0.33 g) were degassed in dioxane/water (5:1, 18 mL) and stirred ON at 110° C. The mixture was filtered. The OL (EtOAc/water) was dried, concentrated, and purified by FC (hexane/EtOAc 7:3) to give Int. 1AF91 tert-butyl 4-(2-fluoro-4-(methoxycarbonyl)-6-methyl-phenyl)-3,6-dihydro-pyridine-1 (2H)-carboxylate (0.65 g). Int. 1AF91 (10 g) was stirred in 1.5 M HCl in dioxane (160 mL) at 0° C. to RT. The residue after concentration was triturated in Et₂O to give Int. 1AF90 methyl 3-fluoro-5-methyl-4-(1,2,3,6-tetrahydropyridin-4-yl)benzoate (13 g, HCl salt).

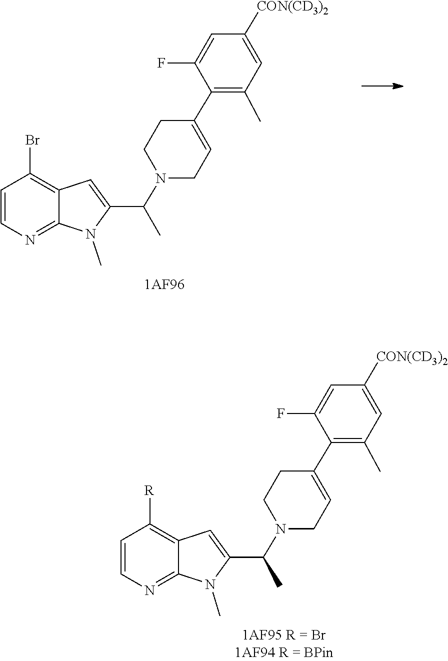

Ints. 1AF88/1AE12 (4.5 g, 5.5 g, HCl salt), Cs₂CO₃ (27 g), and KI (1.4 g) were stirred ON in ACN (50 mL) at 0° C. to RT. The mixture was diluted with water, filtered, and concentrated. The OL (EtOAc/water) was dried and concentrated. The residue was mixed with another batch prepared similarly on 1.5 g scale and purified by FC (pentane/EtOAc 3:7) to give Int. 1AF96 (4.8 g). Int. 1AF96 (5.0 g) was resolved by SFC using a Lux Cellulose.2 250×30 mm 5 μm column operated at 30° C. and an eluent of 70% CO₂ and 30% IPA (90 g/min) and a back pressure of 120 bar to give Int. 1AF95 (1.5 g, peak 2). Int. 1AF95 (0.5 g), B₂Pin₂ (0.38 g), KOAc (0.29 g), and PdDPPFCl₂-DCM (81 mg) were degassed in dioxane (5 mL) and stirred 1 h at 120° C. under MW conditions. The mixture was concentrated and triturated in pentane/Et₂O to give Int. 1AF94 3-fluoro-5-methyl-4-[1-[(1S)-1-[1-methyl-4-(4,4,5,5-tetramethyl-1,3,2-dioxaborolan-2-yl)pyrrolo[2,3-b]-pyridin-2-yl]ethyl]-3,6-dihydro-2H-pyridin-4-yl]-N,N-bis(trideuteriomethyl)benzamide (0.6 g).

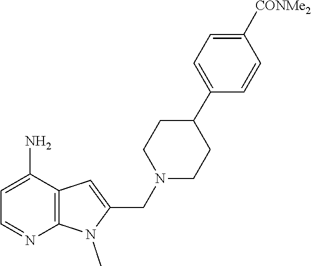

Int. 1ABI (0.29 g), CuI (0.12 g), and NaN₃ (83 mg) were degassed in DMSO/DMEDA (14.4:1, 1.4 mL) and stirred at 100° C. The OL (sat. aq. NH₄Cl/EtOAc) was washed with water, brine, dried, concentrated, HPLC-purified, and triturated in MTBE to give Int. 1C1 4-[1-[(4-Amino-1-methyl-pyrrolo[2,3-b]pyridin-2-yl)methyl]-4-piperidyl]-N,N-dimethyl-benzamide (0.22 g).

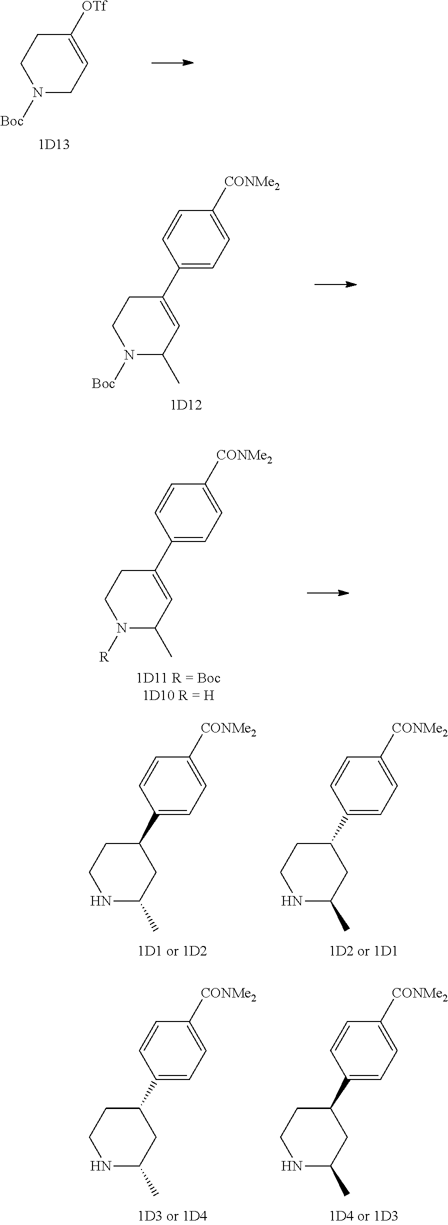

2.0M LDA in THF (28.1 mL) was added to a solution of tert-butyl 2-methyl-4-oxo-piperidine-1-carboxylate (10.0 g) in THF (200 mL) −78° C. The mixture was stirred for 0.3 h at −78° C. 1,1,1-Trifluoro-N-phenyl-N-((trifluoro-methyl)sulfonyl)methane-sulfonamide (20 g in THF (10 mL)) was added and stirring continued 3 h at −78° C. to RT. The OL (aq. NH₄Cl/EtOAc) was washed with water and brine, dried, and concentrated to give Int. 1D13 tert-butyl 6-methyl-4-(trifluoromethyl-sulfonyloxy)-3,6-dihydro-2H-pyridine-1-carboxylate (18.0 g). Int. 1D13 (10 g), Na₂CO₃ (7.67 g), (4-(dimethyl-carbamoyl)phenyl)boronic acid (8.38 g), and PdDPPFCl₂-DCM (1.20 g) were degassed in toluene/water (5:1, 120 mL) and refluxed ON. The OL (water/EtOAc) was dried, concentrated, and purified by FC (hexane/EtOAc 7:3) to give Int. 1D12 tert-butyl 4-[4-(dimethyl-carbamoyl)phenyl]-6-methyl-3,6-dihydro-2H-pyridine-1-carboxylate (5.2 g). 5.0 g of this material was hydrogenated ON using 10% Pd/C (2.50 g) and H₂ (50-60 psi) in EtOH (75 mL), filtered, concentrated, and purified by FC (hexane/EtOAc 35:65) to give Int. 1D11 tert-butyl 4-[4-(dimethylcarbamoyl)phenyl]-2-methyl-piperidine-1-carboxylate (4.0 g). 5.0 g of this material was stirred ON in DCM/TFA (3.6:1, 26 mL) at 0° C. to RT, concentrated, and triturated in Et₂O/pentane to give Int. 1D10 N,N-dimethyl-4-(2-methyl-4-piperidyl)benzamide (4.0 g, TFA salt). Int. 1D10 (10.8 g) was separated into the racemic cis and trans diastereomers by SFC using a Chiral Art Cellulose SC 250×30 5 μm column operated at 30° C. and an eluent of 60% CO₂ and 40% MeOH containing 0.5% HNEt₂ (90 g/min) and a back pressure of 120 bar to give the cis racemate (first two peaks; 3.0 g) and the trans racemate (last two peaks; 7.0 g). The trans racemate (7.0 g) was resolved by SFC on a Sepiatec instrument fitted with a Chiralpak IK 250×30 5 μm column operated at 30° C. and an eluent of 70% CO₂ and 30% MeOH containing 0.5% NH₃ at a (90 g/min) and a back pressure of 100 bar to give Int. 1D1 N,N-dimethyl-4-((2S,4R)-2-methylpiperidin-4-yl)benzamide (0.95 g, first peak) and Int. 1D2 N,N-dimethyl-4-((2R,4S)-2-methylpiperidin-4-yl)benzamide (1.2 g, second peak). The absolute configuration was determined by VCD for Int. 1D1 and 1D2. The cis racemate (3.0 g) was resolved by SFC using a Chiralpak IGK 250×30 5 μm column operated at 30° C. and an eluent MeOH containing 0.5% NH₃ (30 mL/min) and a back pressure of 100 bar to give Int. 1D3 N,N-dimethyl-4-((2R,4R)-2-methylpiperidin-4-yl)benzamide (0.70 g, first peak) and Int. 1D4 N,N-dimethyl-4-((2S,4S)-2-methyl-piperidin-4-yl)benzamide (0.90 g, second peak). The absolute configuration was determined by VCD for Int. 1D3 and 1D4.

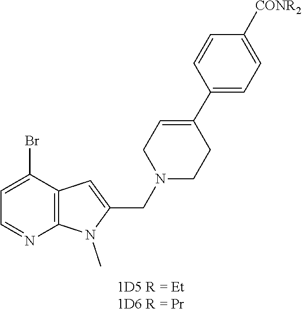

Int. 1CA4 (20 mg), HNEt₂ (24 μL), and HATU (27 mg) were stirred 0.25 h in DMF (1 mL). The OL (sat. aq. NaHCO₃/EtOAc) was washed with brine, dried, and concentrated to give Int. 1D5 4-(1-((4-Bromo-1-methyl-1H-pyrrolo[2,3-b]pyridin-2-yl)methyl)piperidin-4-yl)-N,N-diethylbenzamide (30 mg). Int. 1D6 4-(1-((4-bromo-1-methyl-1H-pyrrolo[2,3-b]pyridin-2-yl)methyl)piperidin-4-yl)-N,N-dipropyl-benzamide (37 mg, was prepared similarly from HNPr₂.

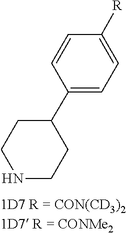

1-(1,1-Dimethylethyl) 4-(4-carboxyphenyl)-1-piperidinecarboxylate (5.1 g), HATU (13 g), and HN(CD₃)₂ (2.5 g, HCl salt) were stirred ON in DMF/DIPEA (1.8:1, 47 mL). Water was added to precipitate solid that was dissolved in DCM/MeOH (9:1), dried, concentrated, and purified by FC (hexane/EtOAc 2:3) to give tert-butyl 4-[4-[bis-(trideuteriomethyl)carbamoyl]phenyl]piperidine-1-carboxylate (4.8 g). 1.1 g of this material was stirred ON in DCM/TFA (3.6:1, 26 mL) at 0° C. to RT, concentrated, and triturated in Et₂O to give Int. 1D7 N,N-Bis(methyl-d₃)-4-(112-piperidin-4-yl)-benzamide (0.81 g, TFA salt). Int. 1D7′ N,N-dimethyl-4-(4-piperidyl)benzamide (9 g, HCl salt) was prepared similarly from 1-(1,1-dimethylethyl) 4-(4-carboxyphenyl)-1-piperidine-carboxylate (17 g) and HN(CH₃)₂ (13.6 g, HCl salt).

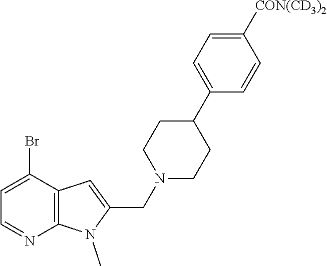

Int. 1AC4 (4.0 g), EDC (4.7 g, HCl salt), HOBt (2.5 g), and HN(CD₃)₂ (1.4 g, HCl salt) were stirred ON in DMF/DIPEA (8.2:1, 89 mL). The residue after concentration was purified by FC (EtOAc/hexane) to give Int. 1D8 4-(1-((4-bromo-1-methyl-1H-pyrrolo[2,3-b]pyridin-2-yl)methyl)piperidin-4-yl)-N,N-bis(methyl-d₃)benzamide (2.7 g).

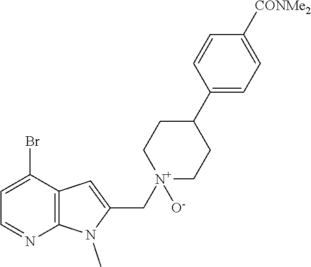

Int. 1ABI (1 g) mCPBA (0.59 g) were stirred 2 h in DCM (10 mL) at 0° C. The OL (sat. aq. NaHCO₃/DCM) was dried and concentrated. The residue was stirred 1 h in TFA, concentrated, and purified by FC (DCM/MeOH 1:0 to 4:1) to give Int. 1D9 4-[1-[(4-bromo-1-methyl-pyrrolo[2,3-b]pyridin-2-yl)methyl]-1-oxido-piperidin-1-ium-4-yl]-N,N-dimethyl-benzamide (0.77 g).

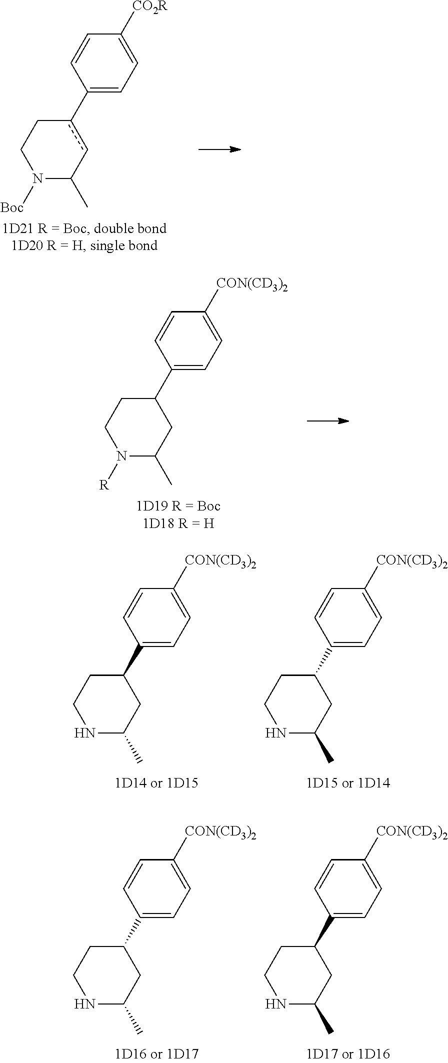

Int. 1D21 tert-butyl 4-(4-(methoxycarbonyl)phenyl)-6-methyl-3,6-dihydropyridine-1 (2H)-carboxylate (5.20 g) was prepared similarly to Int. 1D12 from Int. 1D13 (10.0 g) and (4-(methoxycarbonyl)phenyl)boronic acid (7.82 g). Int. 1D20 tert-butyl 4-(4-(methoxycarbonyl)phenyl)-2-methylpiperidine-1-carboxylate (3.0 g) was prepared similarly to Int. 1D11 from Int. 1D21 (3.5 g). Int. 1D20 (3.0 g) and LiOH—H₂O (1.50 g) were stirred 4 h in THF/MeOH/water (1.3:1:1, 50 mL) at 0° C. to RT and concentrated. The OL (aq. citric acid/EtOAc) was dried and concentrated to give 4-(1-tert-butoxy-carbonyl-2-methyl-4-piperidyl)benzoic acid (1.50 g) 6.0 g of this material, HN(CD)₃)₂ (2.47 g, HCl salt), HOBt (1.90 g), and EDC-HCl (2.70 g) were stirred ON in DMF/DIPEA (1.9:1, 46 mL) at 0° C. to RT. The OL (water/EtOAc) was dried and concentrated to give Int. 1D19 tert-butyl 4-(4-(bis(methyl-d₃)carbamoyl)-phenyl)-2-methylpiperidine-1-carboxylate (4.50 g). 3.5 g of this material was stirred 4 h in DCM/TFA (2.5:1, 70 mL) at 0° C. to RT, concentrated, and triturated in Et₂O/pentane to give Int. 1D18 4-(2-methyl-4-piperidyl)-N,N-bis(trideuteriomethyl)benzamide (3.50 g, TFA salt). Int. 1D18 was separated into Ints. 1D14 N,N-bis(methyl-d₃)-4-((2S,4R)-2-methylpiperidin-4-yl)benzamide, Int. 1D15 N,N-bis-(methyl-d₃)-4-((2R,4S)-2-methylpiperidin-4-yl)benzamide, Int. 1D16 N,N-bis(methyl-d₃)-4-((2R,4R)-2-methylpiperidin-4-yl)benzamide, and Int. 1D17 N,N-bis(methyl-d₃)-4-((2S,4S)-2-methyl-piperidin-4-yl)benzamide as described for Ints. 1D1-1D4. The absolute configurations of these compounds were not determined. The absolute configurations of these compounds were determined by comparison to RT on chiral chromatography vs. Int. 1D1-1D4.

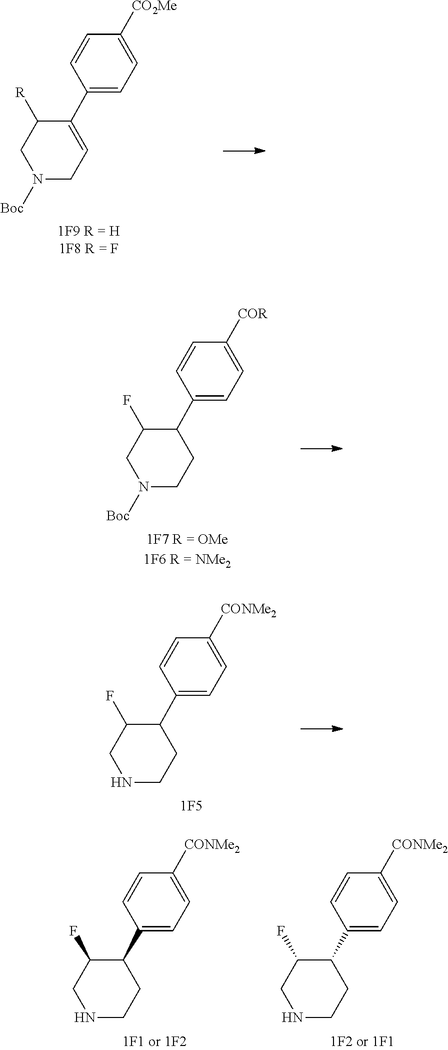

Methyl 4-bromobenzoate (25 g), PdDPPFCl₂-DCM (4.7 g), NaHCO₃ (49 g), and tert-butyl 4-(4,4,5,5-tetramethyl-1,3,2-dioxaborolan-2-yl)-3,6-dihydropyridine-1 (2H)-carboxylate (39 g) were degassed in 1,4-dioxane/water (5:1, 0.6 L) and stirred ON at 110° C. The OL (water/EtOAc) was washed with brine, dried, concentrated, and purified by FC (pentane/EtOAc 99:1) to give Int. 1F9 tert-butyl 4-(4-(methoxycarbonyl)-phenyl)-3,6-dihydro-pyridine-1 (2H)-carboxylate (18 g). Int. 1F9 (38 g) and SelectFluor (7.2 g) were stirred 1 h in ACN/water (3:1, 0.64 L). SelectFluor (2.1 g) was added and stirring continued 1 h. The OL (EtOAc/sat. aq. NaHCO₃) was dried, concentrated, and purified by FC (hexane/EtOAc 85:15) to give Int. 1F8 tert-butyl-3-fluoro-4-(4-methoxy-carbonylphenyl)-3,6-dihydro-2H-pyridine-1-carboxylate (6.0 g). Int. 1F8 (4.8 g) was hydrogenated ON using 10% Pd/C (0.48 g) in EtOAc (200 mL), filtered, concentrated, and purified by FC (hexane/EtOAc 9:1) to give Int. 1F7 tert-butyl-3-fluoro-4-(4-methoxycarbonylphenyl)piperidine-1-carboxylate (3.2 g). Int. 1F7 (3.5 g) and LiOH—H₂O (1.4 g) were stirred ON in MeOH/THF/H₂O (3:2:1; 25 mL) at 0° C. to RT and concentrated. The OL (10% MeOH in DCM/aq. citric acid) was dried and concentrated to give cis-4-(1-(tert-butoxycarbonyl)-3-fluoro-piperidin-4-yl)benzoic acid (2.1 g). 2.5 of this compound, HATU (4.4 g), and HN(CH₃)₂ (1.9 g, HCl salt) were stirred ON in DMF/DIPEA (2.4:1, 35 mL) at 0° C. to RT. The OL (water/5% MeOH in DCM) was dried, concentrated, and purified by FC (DCM/MeOH 95:5) to give Int. 1F6 cis-tert-butyl-4-[4-(dimethylcarbamoyl)-phenyl]-3-fluoro-piperidine-1-carboxylate (2.4 g). Int. 1F6 (1.5 g) was stirred ON in DCM/TFA (3,8:1, 13 mL) at 0° C. to RT and concentrated. The OL (aq. NaHCO₃/Et₂O and 10% MeOH in DCM) was dried and concentrated to give Int. 1F5 N,N-dimethyl-4-[cis-3-fluoro-4-piperidyl]-benzamide (1.19 g). This material was resolved by SFC using a Lux Cellulose-4 250×30 5 μm column operated at 30° C. and an eluent of 70% CO₂ and 30% 0.5% iso-propyl amine in IPA (100 g/min) and a back pressure of 100 bar to give Int. 1F1 (0.47 g) and Int. 1F2 (0.50 g) 4-((3S,4R)-3-Fluoropiperidin-4-yl)-N,N-dimethylbenzamide and 4-((3R,4S)-3-fluoropiperidin-4-yl)-N,N-dimethyl-benzamide. The absolute configurations of these compounds were not determined.

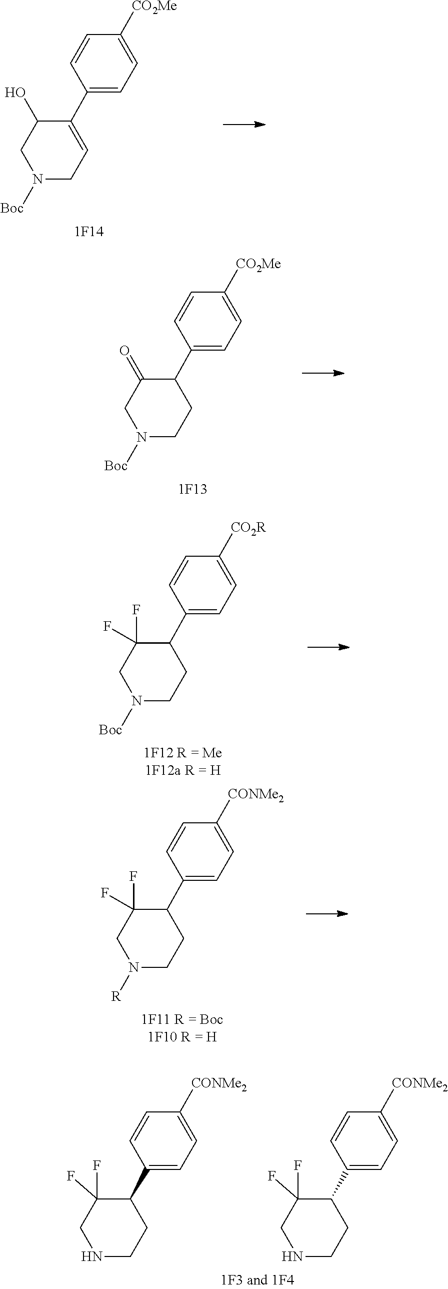

Int. 1F9 (10 g) was stirred ON in THF/2M BH₃-DMS in THF (11.6:1, 217 mL) at 0° C. to RT. 2M aq. NaOH (38 mL) and 30% aq. H₂O₂ (5.9 mL) were added and stirring was continued at 0° C. to RT over 1 h. The OL (sat. aq. NaS₂O₃/EtOAc) was washed with water and brine, dried, concentrated, and purified by FC (hexane/EtOAc 7:3) to give Int. 1F14 tert-butyl 3-hydroxy-4-(4-(methoxy-carbonyl)phenyl)piperidine-1-carboxylate (7.4 g). Int. 1F14 (8.0 g) and DMP (5.1 g) were stirred ON in DCM (200 mL) at 0° C. to RT. The OL (water/DCM) was dried, concentrated, and purified by FC (hexane/EtOAc 9:1) to give Int. 1F13 tert-butyl 4-(4-methoxy-carbonyl-phenyl)-3-oxo-piperidine-1-carboxylate (5.2 g). Int. 1F13 (5.0 g) was stirred ON in DCM/50% Deoxo-Fluor in THF (12.2:1, 108 mL) at −78° C. to RT ON. The OL (aq. NaHCO₃/DCM) was washed with sat. aq. citric acid, dried, concentrated, and purified by FC (hexane) to give Int. 1F12 tert-butyl 3,3-difluoro-4-(4-methoxycarbonyl-phenyl)piperidine-1-carboxylate (2.5 g). This material and LiOH—H₂O (1.5 g) were stirred in MeOH/THF/H₂O (3:3:1; 60 mL) at 0° C. to RT over 4 h, concentrated, and triturated in dilute aq. citric acid to give Int. 1F12a 4-(1-tert-butoxycarbonyl-3,3-difluoro-4-piperidyl)benzoic acid (2.3 g). Int. 1F12a (4.8 g), HATU (6.4 g), and HN(CH₃)₂ (2.3 g, HCl salt) were stirred ON in DMF/DIPEA (13.2:1, 108 mL) at 0° C. to RT. The OL (water/EtOAc) was dried, concentrated, and purified by FC (hexane/EtOAc 1:4) to give Int. 1F11 tert-butyl 4-(4-(dimethylcarbamoyl)-phenyl)-3,3-difluoro-piperidine-1-carboxylate (4.5 g). Int. 1F11 (4.2 g) was stirred ON in DCM/TFA (27:1, 83 mL) at 0° C. to RT and concentrated. The OL (5% MeOH in DCM/sat. aq. NaHCO₃) was dried and concentrated to give Int. 1F10 4-(3,3-difluoro-4-piperidyl)-N,N-dimethyl-benzamide (3 g). This material was resolved by SFC using a Chiralpak IG 250×30 5 μm column operated at 30° C. and an eluent of 60% CO₂ and 40% MeOH (80 g/min) and a back pressure of 60 bar to give Int. 1F3 (1.1 g) and Int. 1F4 (1.0 g) (R)-4-(3,3-Difluoropiperidin-4-yl)-N,N-dimethylbenzamide and (S)-4-(3,3-difluoropiperidin-4-yl)-N,N-dimethylbenzamide. The absolute configurations of these compounds were not determined.

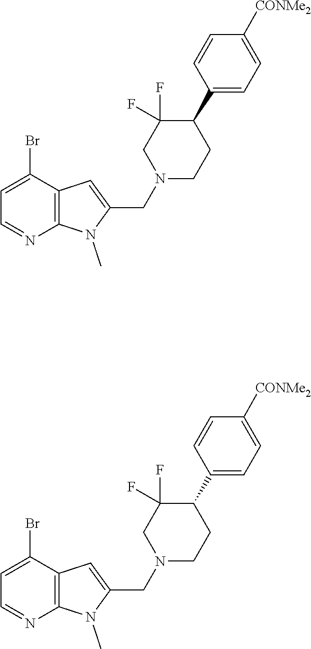

Ints. 1J1/1F3 (45 mg/60 mg), Cs₂CO₃ (169 mg), NaI (26 mg) were stirred in DMF (1 mL) for 0.5 h and HPLC-purified to give Int. 1F15 (48 mg). Int. 1F16 was prepared similarly from Int. 1F4. Ints. 1F15 and 1F16 (R)-4-(1-((4-bromo-1-methyl-1H-pyrrolo[2,3-b]pyridin-2-yl)methyl)-3,3-difluoropiperidin-4-yl)-N,N-dimethylbenzamide and (S)-4-(1-((4-bromo-1-methyl-1H-pyrrolo[2,3-b]pyridin-2-yl)methyl)-3,3-difluoropiperidin-4-yl)-N,N-dimethylbenzamide. The absolute configurations were not determined for these compounds.

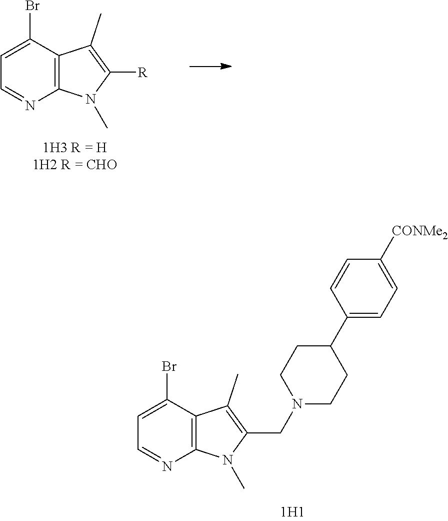

4-Bromo-3-methyl-1H-pyrrolo[2,3-b]pyridine (0.51 g) and NaH (0.12 g) were stirred 1 h in DMF (5 mL) at 0° C. before MeI (0.3 mL) was added and stirring continued for 0.3 h. The OL (water/EtOAc/Et₂O) was washed with brine, dried, concentrated to give Int. 1H3 4-bromo-1,3-dimethyl-pyrrolo[2,3-b]pyridine (0.53 g). This material was stirred 2 h in THF/1.0 M LDA in THF (2.9:1, 13.5 mL) −78° C. before DMF (1 mL) was added and stirring continued 2 h at −78° C. to RT. The OL (sat. aq. NH₄Cl/EtOAc) was washed with brine, dried, concentrated, and purified by FC (heptane/EtOAc 1:0 to 1:1) to give Int. 1H2 4-bromo-1,3-dimethyl-pyrrolo[2,3-b]pyridine-2-carbaldehyde (0.17 g). Int. 1H2 (30 mg) and N,N-dimethyl-4-(piperidin-4-yl)benzamide (42 mg) were stirred 0.5 h in DCE (2 mL). STAB (57 mg) was added and stirring continued ON. The OL (sat. aq. NaHCO₃/DCM.) was washed with brine, dried, concentrated, and purified by FC (DCM/MeOH 1:0 to 9:1) to give Int. 1H1 4-[1-[(4-bromo-1,3-dimethyl-pyrrolo[2,3-b]pyridin-2-yl)methyl]-4-piperidyl]-N,N-dimethyl-benzamide (30 mg).

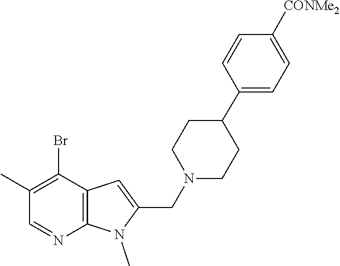

Int. 1I1 4-(1-((4-bromo-1,5-dimethyl-1H-pyrrolo[2,3-b]pyridin-2-yl)methyl)piperidin-4-yl)-N,N-dimethylbenzamide (0.16 g) as prepared similarly to Int. 1H1 from 4-bromo-5-methyl-1H-pyrrolo[2,3-b]pyridine (0.51 g) and N,N-dimethyl-4-(piperidin-4-yl)benzamide (0.18 g).

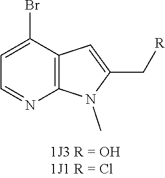

Int. 1AB8 (30 g) and NaBH₄ (19 g) were stirred 2 h in MeOH (300 mL) at 0° C. to RT. The OL (aq. Na₂S₂O₃/EtOAc) was dried, concentrated, and purified by FC (pentane/EtOAc 1:1 to 2:3) to give Int. 1J3 (4-bromo-1-methyl-pyrrolo[2,3-b]pyridin-2-yl)methanol. Int. 1J3 (2.5 g) was stirred ON in DCM/Et₃N/MsCl (21.4:5.2:1, 39 mL) at 0° C. to RT and filtered. The OL layer (EtOAc/water) was washed with brine, dried, and concentrated to give Int. 1J1 4-bromo-2-(chloromethyl)-1-methyl-1H-pyrrolo[2,3-b]pyridine (2.0 g).

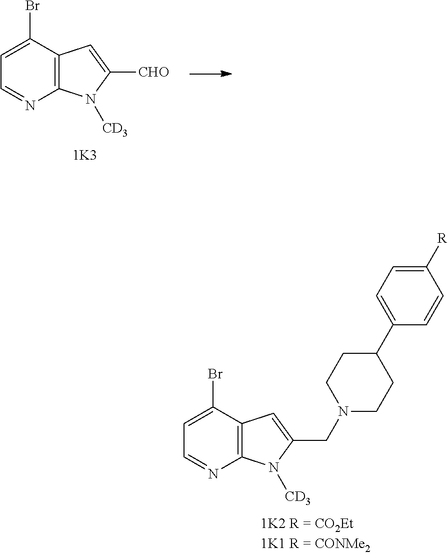

4-Bromo-1-(trideuteriomethyl)pyrrolo[2,3-b]pyridine (0.40 g) was stirred 2 h in THF/2M LDA in THF (7.1:1, 11.4 mL) at −78° C. DMF (0.7 mL) was added and stirring continued 2 h at −78° C. to RT and diluted with water to precipitate Int. 1K3 4-bromo-1-(trideuterio-methyl)pyrrolo[2,3-b]pyridine-2-carbaldehyde (0.35 g). Int. 1K3 (0.17 g) and methyl 4-(piperidin-4-yl)benzoate (0.16 g) were stirred 3 h in DCE/DIPEA (28.3:1, 15.5 mL). STAB (0.26 g) was added and stirring continued ON. The OL (water/DCM) was washed brine, dried, concentrated, and purified by FC (pentane/EtOAc 4:1) to give Int. 1K2 methyl 4-[1-[[4-bromo-1-(trideuteriomethyl)pyrrolo-[2,3-b]pyridin-2-yl]methyl]-4-piperidyl]-benzoate (0.14 g). Int. 1K2 (50 mg) was stirred 0.25 h in THF/MeOH/2M aq. NaOH (7.1:3.6:1, 3.3 mL). pH was adjusted to 5 with 1M aq. HCl. The mixture was concentrated. The residue and HATU (85 mg) were dissolved in DMF/DIPEA (25.6:1, 2.1 mL) before HN(CH₃)₂ (14 mg; HCl salt) was added and stirring continued 72 h. The OL (water/DCM) was dried, concentrated, and purified by FC (DCM/MeOH 1:to 9:1) to give Int. 1K1 4-[1-[[4-bromo-1-(trideuteriomethyl)pyrrolo[2,3-b]pyridin-2-yl]-methyl]-4-piperidyl]-N,N-dimethyl-benzamide (39 mg).

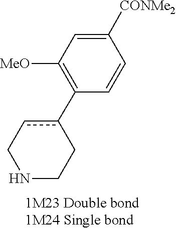

4-Bromo-3-methoxybenzoic acid (3.0 g), HN(CH₃)₂ (1.6 g, HCl salt), and HATU (5.92 g) were stirred ON in DCM/DIPEA (7.4:1, 57 mL), concentrated, and HPLC-purified to give 4-bromo-3-methoxy-N,N-dimethyl-benzamide (2.5 g). 1.0 g of this material, tert-butyl 4-(4,4,5,5-tetramethyl-1,3,2-dioxaborolan-2-yl)-3,6-dihydropyridine-1 (2H)-carboxylate (1.44 g), K₂CO₃ (2.14 g), and PdDPPFCl₂-DCM (0.16 g) were degassed in dioxane/water (4:1, 10 mL) and stirred at 100° C. ON. The OL (water/EtOAc) was washed with brine, dried, concentrated, and purified by FC (hexane to EtOAc) to give tert-butyl 4-(4-(dimethylcarbamoyl)-2-methoxy-phenyl)-3,6-dihydropyridine-1 (2H)-carboxylate (1.10 g). 0.6 g of this material was hydrogenated ON using 10% Pd/C (0.2 g) in MeOH (10 mL), filtered, concentrated, stirred in 4M HCl in dioxane (10 mL) ON, concentrated, and HPLC-purified to give Int. 1M24 3-methoxy-N,N-dimethyl-4-(piperidin-4-yl)benzamide (0.23 g). tert-Butyl 4-(4-(dimethylcarba-moyl)-2-methoxyphenyl)-3,6-dihydropyridine-1 (2H)-carboxylate (0.50 g) was stirred ON in 4M HCl in dioxane (10 mL), concentrated, and HPLC-purified to give Int. 1M23 3-methoxy-N,N-dimethyl-4-(1,2,3,6-tetrahydropyridin-4-yl)benzamide (0.17 g).

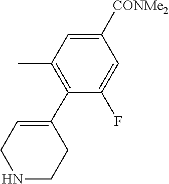

NaNO₂ (6.1 g) and 4-amino-3-fluoro-5-methyl-benzoic acid (15.0 g) were stirred 0.3 h in 48% aq. HBr (150 mL) at 0° C. CuBr (13.0 g) was added and stirring continued 4 h. The OL (aq. NaHCO₃/MTBE) was washed with water and concentrated to give 4-bromo-3-fluoro-5-methyl-benzoic acid (8.50 g). 4-Bromo-3-fluoro-5-methyl-benzoic acid (15.0 g) was stirred 5 h in DCM/SOCl₂ (83:1, 150 mL), and concentrated. The residue and HNMe₂ (3.1 g, HCl salt) were stirred 5 h DCM/Et₃N (13.6:1, 160 mL) The OL (water/DCM) was concentrated. The residue, K₂CO₃ (7.3 g), PdPDPPFCl₂-DCM (0.15 g), and tert-butyl 4-(4,4,5,5-tetramethyl-1,3,2-dioxaborolan-2-yl)-3,6-dihydro-pyridine-1 (2H)-carboxylate (6.0 g) were degassed dioxane (150 mL) and stirred 48 h at 90° C.

The OL (water/dioxane) was concentrated, stirred 0.5 h in THF/4M HCl in dioxane (1:1, 100 mL), concentrated, and HPLC-purified to give Int. 1M25 3-fluoro-N,N,5-trimethyl-4-(1,2,3,6-tetrahydropyridin-4-yl)benzamide (0.22 g).

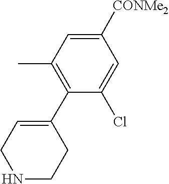

Int. 1M26 3-chloro-N,N,5-trimethyl-4-(1,2,3,6-tetrahydropyridin-4-yl)benzamide (0.11 g) was prepared similarly to Int. 1M25 from 4-bromo-3-chloro-5-methyl-benzoic acid (15 g).

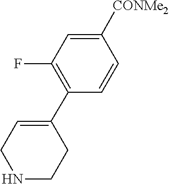

4-Bromo-3-fluoro-N,N-dimethyl-benzamide (1.0 g), tert-butyl 4-(4,4,5,5-tetramethyl-1,3,2-dioxa-borolan-2-yl)-3,6-dihydro-2H-pyridine-1-carboxylate (1.41 g), Na₂CO₃ (1.61 g), and PdDPPFCl₂-DCM (0.16 g) were degassed in dioxane/water (4:1, 10 mL) and stirred ON at 100° C. The OL (water/EtOAc) was washed with brine, dried, concentrated, and purified by FC (hexane to EtOAc) to give tert-butyl 4-[4-(dimethylcarbamo-yl)-2-fluoro-phenyl]-3,6-dihydro-2H-pyridine-1-carboxylate (1.20 g). 0.6 g of this material was stirred ON in 4M HCl in dioxane (10 mL). The mixture was concentrated and HPLC-purified to give Int. 1M29 3-fluoro-N,N-dimethyl-4-(1,2,3,6-tetrahydropyridin-4-yl)-benzamide (0.22 g).

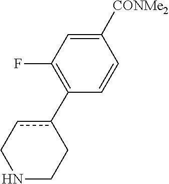

4-Bromo-3-chloro-N,N-dimethyl-benzamide (5.0 g), tert-butyl 4-(4,4,5,5-tetramethyl-1,3,2-dioxaborolan-2-yl)-3,6-dihydropyridine-1 (2H)-carboxylate (0.71 g), Na₂CO₃ (8.1 g), and PdDPPFC₂-DCM (0.78 g) were degassed in dioxane/water (4:1, 10 mL) and stirred at 100° C. ON. The OL (water/EtOAc) was dried, concentrated, and purified by FC (hexane to EtOAc) to give tert-butyl 4-[2-chloro-4-(dimethylcarbamoyl)phenyl]-3,6-dihydro-2H-pyridine-1-carboxylate (1.10 g). This material was hydrogenated ON using 10% Pt/C (1.1 g) in MeOH (10 mL), filtered, and concentrated to give tert-butyl 4-[2-chloro-4-(dimethylcarbamoyl)phenyl]-piperidine-1-carboxylate (0.60 g). This material was stirred ON in 4M HCl in dioxane (10 mL), concentrated, and HPLC-purified to give Int. 1M44 3-chloro-N,N-dimethyl-4-(piperidin-4-yl)benzamide (0.15 g). tert-Butyl 4-[2-chloro-4-(dimethyl-carbamoyl)phenyl]-3,6-dihydro-2H-pyridine-1-carboxylate (0.60 g) was stirred ON in 4M HCl in dioxane (10 mL), concentrated, and HPLC-purified to give Int. 1M43 3-chloro-N,N-dimethyl-4-(1,2,3,6-tetrahydropyridin-4-yl)benzamide (0.22 g).

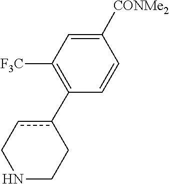

4-Bromo-3-(trifluoromethyl)benzoic acid (3.0 g), HN(CH₃)₂ (1.36 g, HCl salt) and HATU (5.1 g) stirred ON in DCM/DIPEA (8.6:1, 56 mL), concentrated, and HPLC-purified to give 4-bromo-N,N-dimethyl-3-(trifluoro-methyl)benzamide (2.40 g). 1.0 g of this material, tert-butyl 4-(4,4,5,5-tetramethyl-1,3,2-dioxaborolan-2-yl)-3,6-dihydro-pyridine-1 (2H)-carboxylate (1.25 g), K₂CO₃ (1.87 g), and PdDPPFCl₂-DCM (0.14 g) were degassed in dioxane/water (4:1, 10 mL) and stirred ON at 100° C. The OL (water/EtOAc) was washed with brine, dried, concentrated, and purified by FC (hexane to EtOAc) to obtain tert-butyl 4-[4-(dimethyl-carbamoyl)-2-(trifluoromethyl)phenyl]-3,6-dihydro-2H-pyridine-1-carboxylate (1.04 g). 0.50 g of this material was stirred ON in 4M HCl in dioxane (10 mL), filtered, and HPLC-purified to give Int. 1M45 N,N-dimethyl-4-(1,2,3,6-tetrahydropyridin-4-yl)-3-(trifluoro-methyl)benzamide (0.14 g). tert-Butyl 4-[4-(dimethyl-carbamoyl)-2-(trifluoromethyl)phenyl]-3,6-dihydro-2H-pyridine-1-carboxylate (0.50 g) was hydrogenated ON using 10% Pd/C (0.5 g) in MeOH (10 mL). The mixture was filtered. The residue after concentration was stirred ON in 4M HCl in dioxane (10 mL), concentrated, and HPLC-purified to give Int. 1M46 N,N-dimethyl-4-(piperidin-4-yl)-3-(trifluoromethyl)benzamide (0.23 g).

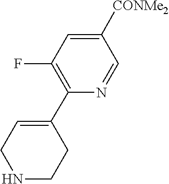

6-Chloro-5-fluoro-pyridine-3-carboxylic acid (3.0 g), HN(CH₃)₂ (1.53 g, HCl salt), and HATU (7.15 g) were stirred ON in DCM/DIPEA (5.6:1, 59 mL), concentrated, and purified by FC (hexane to EtOAc) to give 6-chloro-5-fluoro-N,N-dimethyl-pyridine-3-carboxamide (3.1 g). This material, tert-butyl 4-(4,4,5,5-tetramethyl-1,3,2-dioxa-borolan-2-yl)-3,6-dihydropyridine-1 (2H)-carboxylate (5.20 g), K₃CO₃ (8.50 g), and PdDPPFCl₂-DCM (0.62 g) were degassed in dioxane/water (4:1, 50 mL) and stirred ON at 100° C. The OL (EtOAc/water) was washed with brine, dried, concentrated, and purified by FC (hexane to EtOAc) to give tert-Butyl 4-[5-(dimethylcarbamoyl)-3-fluoro-2-pyridyl]-3,6-dihydro-2H-pyridine-1-carboxylate (3.10 g). 0.13 g of this material was stirred 1.5 h in DCM/4M HCl in dioxane (3.6:1, 6.4 mL) and concentrated. The OL (DCM/aq. NaHCO₃) was dried, concentrated, and HPLC-purified to give Int. 1M47 5-fluoro-N,N-dimethyl-6-(1,2,3,6-tetrahydropyridin-4-yl)pyridine-3-carboxamide (20 mg).

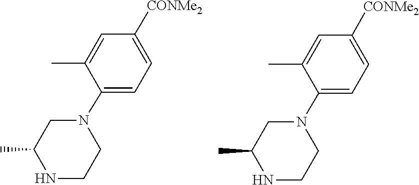

tert-Butyl (S)-2-methylpiperazine-1-carboxylate (1.0 g), ethyl 4-bromo-3-methyl-benzoate (1.46 g), Cs₂CO₃ (4.88 g), Pd₂dba₃ (0.23 g), and XPhos (0.24 g) were degassed in dioxane (20 mL) and stirred at 110° C. ON. The OL (EtOAc/water) was concentrated and purified by FC (hexane to EtOAc) to give tert-butyl (2R)-4-(4-ethoxy-carbonyl-2-methyl-phenyl)-2-methyl-piperazine-1-carboxylate (0.85 g). 0.70 g of this material and LiOH—H2O (0.22 g) were stirred 6 h in THF/water/MeOH (10 mL), concentrated, and triturated in dilute aq. citric acid to give 4-[(3R)-4-tert-butoxy-carbonyl-3-methyl-piperazin-1-yl]-3-methyl-benzoic acid (0.45 g). 4-[(3R)-4-tert-butoxycarbonyl-3-methyl-piperazin-1-yl]-3-methyl-benzoic acid (0.70 g), HNMe₂ (0.26 g, HCl salt), HOBt (0.42 g), and EDC (0.60 g, HCl salt) were stirred ON in DCM/DIPEA (10:1, 11 mL) at 0° C. to RT. The OL (water/DCM) was washed with brine, dried, concentrated, and purified by FC (hexane/EtOAc 3:2) to give tert-butyl (2R)-4-[4-(dimethylcarbamoyl)-2-methyl-phenyl]-2-methyl-piperazine-1-carboxylate (0.45 g). This material was stirred 6 h in DCM/TFA (6:1, 7 mL) at 0° C. to RT, concentrated, and triturated in Et₂O to give Int. 1M49 (R)—N,N,3-trimethyl-4-(3-methylpiperazin-1-yl)-benzamide (0.20 g). Int. 1M59 (S)—N,N,3-trimethyl-4-(3-methylpiperazin-1-yl)-benzamide was prepared similarly from tert-butyl (R)-2-methylpiperazine-1-carboxylate.

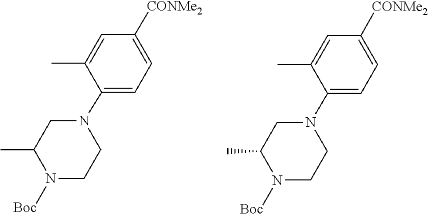

tert-Butyl (S)-2-methylpiperazine-1-carboxylate (2.5 g), NaOtBu (2.4 g), Pd₂dba₃ (0.57 g), Xphos (0.59 g), and 4-bromo-N,N-dimethylbenzamide (3.1 g) were degassed in dioxane (30 mL) and stirred ON at 100° C. The OL (Et₂O/water) was washed with brine, dried, concentrated, and purified by FC (hexane/EtOAc 1:4) to give Int. 1M50 tert-butyl (S)-4-(4-(dimethylcarbamoyl)phenyl)-2-methyl-piperazine-1-carboxylate and tert-butyl (2.4 g). Int. 1M52 (R)-4-(4-(dimethylcarbamoyl)phenyl)-2-methyl-piperazine-1-carboxylate (0.46 g) was prepared similarly from tert-butyl (R)-2-methylpiperazine-1-carboxylate (0.50 g).

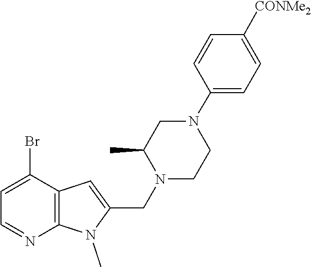

Int. 1M50 (46 mg) was stirred 1 h in DCM/TFA (1:1, 1 mL) and concentrated. The residue and 4 Å MS were stirred ON in DCM/DIPEA (19.5:1, 1.6 mL). Int. 1J1 (23 mg) was added and stirring was continued ON. The OL (DCM/sat. aq. NaHCO₃) was dried, concentrated, and purified by FC (DCM/MeOH 1:0 to 95:5) to give Int. 1M51 (S)-4-(4-((4-bromo-1-methyl-1H-pyrrolo[2,3-b]pyridin-2-yl)methyl)-3-methylpiperazin-1-yl)-N,N-dimethylbenzamide (40 mg).

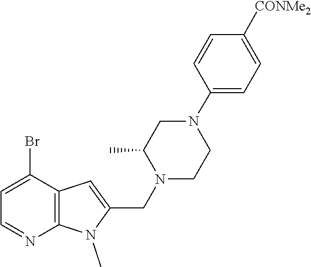

Int. 1M52 (46 mg) was stirred 1 h in DCM/TFA (1:1, 1 mL) and concentrated. The residue, Int. 1AB8 (21 mg), and 4 Å MS were stirred 2 h in DCM/DIPEA (19.5:1, 1.6 mL). STAB (56 mg) was added and stirring continued ON. The OL (water/DCM) was dried, concentrated, and purified by FC (DCM/MeOH 1:0 to 95:5) to give Int. 1M53 (R)-4-(4-((4-bromo-1-methyl-1H-pyrrolo[2,3-b]pyridin-2-yl)methyl)-3-methylpiperazin-1-yl)-N,N-dimethylbenzamide (30 mg).

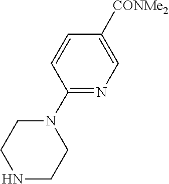

Boc-piperazine (0.40 g), 6-bromo-N,N-dimethylnicotinamide (0.59 g), NaOtBu (0.41 g), Xphos (0.10 g), and Pd₂dba₃ (0.1 g) were degassed in dioxane (4 mL) and stirred at 110° C. ON. The OL (water/EtOAc) was washed with brine, dried, concentrated, and purified by FC (pentane/EtOAc 1:1) to give 0.30 g of tert-butyl 4-(5-(dimethylcarbamoyl)pyridin-2-yl)-piperazine-1-carboxylate. 0.26 g of this material was stirred 1 h in 3M HCl in dioxane (3 mL) at 0° C. to RT and concentrated to give Int. 1M55 N,N-dimethyl-6-(piperazin-1-yl)nicotinamide (0.14 g, HCl salt).

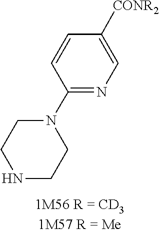

4-(4-tert-Butoxycarbonylpiperazin-1-yl)benzoic acid (1.0 g), HN(CD₃)₂ (0.43 g, HCl salt), HOBt (0.66 g), and EDC (0.94 g, HCl salt) were stirred ON in DCM/DIPEA (15.4:1, 21 mL). The OL (water/DCM) was washed with brine, dried, concentrated, and purified by FC (pentane/EtOAc 1:1) to give tert-butyl 4-(5-(bis(methyl-d₃)carbamoyl)-pyridin-2-yl)piperazine-1-carboxylate (0.95 g). This material was stirred in DCM/TFA (6.7:1, 35 mL), concentrated, and triturated in Et₂O to give Int. 1M56 N,N-bis(methyl-d₃)-6-(piperazin-1-yl)nicotinamide (0.85 g). Int. 1AE20 (14 g) was hydrogenated ON using 10% Pd/C (3.5 g) in MeOH (200 mL), filtered, and concentrated to give tert-butyl 4-[5-(dimethyl-carbamoyl)-2-pyridyl]piperidine-1-carboxylate (11 g). 0.9 g of this material was stirred in DCM/TFA (10:1, 11 mL) at 0° C. to RT, concentrated, and triturated in Et₂O to give Int. 1M57 N,N-dimethyl-6-(piperidin-4-yl)nicotinamide (0.50 g, TFA salt).

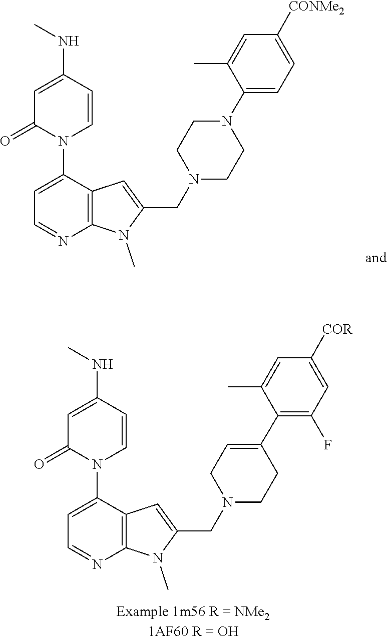

Ints. 1M1/1S7 (15 mg/22 mg, HCl salt) were stirred 0.5 h in DCE (0.5 mL). STAB (25 mg) was added and stirring was continued ON. The mixture was filtered. The filtrate was stirred 0.5 h in TFA (0.5 mL), concentrated, and purified by HPLC to give Example 1m6 N,N,3-Trimethyl-4-(4-((1-methyl-4-(4-(methylamino)-2-oxopyridin-1 (2H)-yl)-1H-pyrrolo[2,3-b]pyridin-2-yl)methyl)piperazin-1-yl)benzamide (6.6 mM in DMSO (0.70 mL). Example 1m56 3-Fluoro-N,N,5-trimethyl-4-(1-((1-methyl-4-(4-(methylamino)-2-oxopyridin-1 (2H)-yl)-1H-pyrrolo[2,3-b]pyridin-2-yl)methyl)-1,2,3,6-tetrahydropyridin-4-yl)benzamide (10 mM in DMSO (1.2 mL)) was prepared similarly to Example 1m6 from Int. 1M25 (17 mg). Example 1m56 (2.4 g) and LiOH—H₂O (1.1 g) were stirred 3 h in THF/water (2.5:1, 28 mL) at 0° C. to 60° C. The residue after concentration was triturated in aq. HCl to give Int. 1M60 3-fluoro-5-methyl-4-(1-((1-methyl-4-(4-(methylamino)-2-oxopyridin-1 (2H)-yl)-1H-pyrrolo[2,3-b]pyridin-2-yl)methyl)-1,2,3,6-tetrahydropyridin-4-yl)benzoic acid (2.4 g).

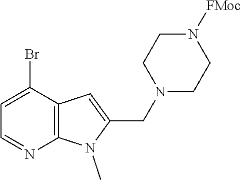

Int. 1AB8 (0.25 g) and 9H-fluoren-9-ylmethyl piperazine-1-carboxylate (0.48 g) were stirred 2 h in DCE/AcOH (100:1, 10 mL). STAB (0.44 g) was added and stirring continued 2 h at 0° C. to RT. The OL (sat. aq. NaHCO₃/DCM) was washed with brine, dried, and concentrated to give Int. 1N2 9H-fluoren-9-ylmethyl 4-[(4-bromo-1-methyl-pyrrolo[2,3-b]pyridin-2-yl)methyl]piperazine-1-carboxylate (0.20 g).

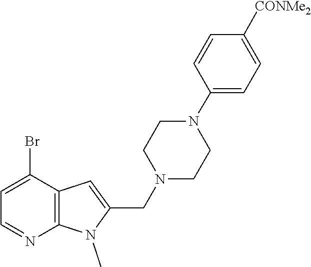

Int. 1AB8 (0.35 g) and N,N-dimethyl-4-(piperazin-1-yl)benzamide (0.40 g, HCl salt) were stirred 4 h in DCE/DIPEA (6.5:1, 11.6 mL). STAB (0.94 g) was added and stirring continued ON. The OL (water/DCM) was washed with brine, dried, concentrated, and purified by FC (hexane/EtOAc 1:4) to give Int. 1N4 4-(4-((4-bromo-1-methyl-1H-pyrrolo[2,3-b]pyridin-2-yl)methyl)piperazin-1-yl)-N,N-dimethyl-benzamide (0.44 g).

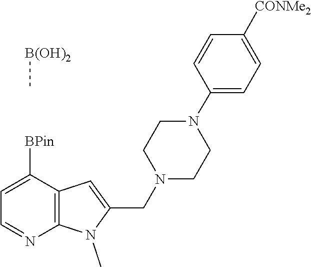

Int. 1N4 (25 mg), KOAc (11 mg), PdDPPFCl₂-DCM (4 mg), and B₂Pin₂ (15 mg) were degassed in dioxane (3 mL) and stirred 4 h at 70° ch. KOAc (11 mg), and B₂Pin₂ (15 mg), and PdDPPFCl₂-DCM (4 mg) were added and stirring continued 4 h at 70° C. and ON at RT. KOAc (11 mg), B₂Pin₂ (15 mg), and PdDPPFCl₂-DCM (4 mg) were added and stirring continued 6 h at 90° C. The mixture was filtered and HPLC-purified to give Int. 1N5 N,N-dimethyl-4-[4-[[1-methyl-4-(4,4,5,5-tetramethyl-1,3,2-dioxa-borolan-2-yl)pyrrolo[2,3-b]pyridin-2-yl]methyl]piperazin-1-yl]benzamide and (2-((4-(4-(dimethyl-carbamoyl)-phenyl)piperazin-1-yl)methyl)-1-methyl-1H-pyrrolo[2,3-b]pyridin-4-yl)boronic acid.

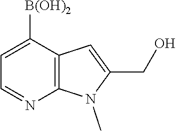

Int. 1J3 (1.0 g), B₂Pin₂ (1.58 g), Pd(PPh₃)₂Cl₂ (0.29 g), and KOAc (1.02 g) were degassed dioxane (10 mL) and stirred 3 h at 100° C. and concentrated. The residue decanted with pentane and concentrated to give Int. 1O3 [2-(hydroxymethyl)-1-methyl-pyrrolo[2,3-b]pyridin-4-yl]boronic acid (0.8 g).

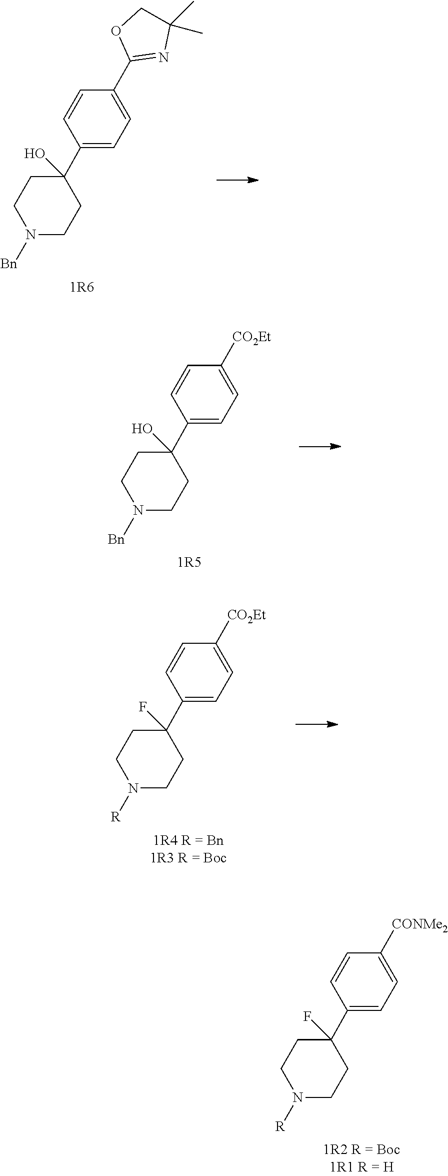

2-(4-Bromophenyl)-4,4-dimethyl-4,5-dihydrooxazole (10 g) and Mg turnings (1.1 g) and I₂ (50 mg) were refluxed 1 h in THF (50 mL). 1-Benzylpiperidin-4-one (8.0 mL) was added before refluxing 3 h. The OL (sat. aq. NH₄Cl/MTBE) was washed with water, brine, dried, and concentrated to give Int. 1R6 1-benzyl-4-(4-(4,4-dimethyl-4,5-dihydro-oxazol-2-yl)phenyl)piperidin-4-ol. Int. 1R6 (7.0 g) was stirred ON in EtOH/96% H₂SO₄ (10:1, 400 mL) at 90° C. and concentrated. The OL (sat. aq. NaHCO₃/DCM) was dried, concentrated, and purified by FC (DCM/2M NH₃ in MeOH 1:0 to 4:1) to give Int. 1R5 ethyl 4-(1-benzyl-4-hydroxypiperidin-4-yl)-benzoate. Int. 1R5 (0.06 g) was dissolved in DCM (10 mL) at −78° C. DAST (0.28 mL) was added and stirring continued 2 h at −78° C. to RT over 2 h. The OL (DCM/water) was washed with brine, dried, concentrated, and HPLC-purified to give Int. 1R4 ethyl 4-(1-benzyl-4-fluoro-piperidin-4-yl)benzoate. Int. 1R4 (2.0 g) was stirred 3 h in DCM (40 mL) and 1-chloroethyl carbono-chloridate (0.76 mL) at 0° C. to RT and concentrated. The residue was refluxed 0.5 h in MeOH (20 mL) and concentrated. The residue and Boc₂O (1.56 g) were stirred ON in DCM/Et₃N (14.7:1, 27 mL). The OL (EtOAc/water) was washed with brine, dried, concentrated, and purified by FC (heptane/EtOAc 1:0 to 1:1) to give 4-(1-(tert-butoxycarbonyl)-4-fluoropiperidin-4-yl)benzoic acid. 0.77 g of this material, HATU (1.18 g), and HN(CH₃)₂ (0.29 g, HCl salt) were stirred 1 h in DMF/DIPEA (5:1, 10 mL). The OL (EtOAc/water) was dried, concentrated, and purified by FC (heptane to EtOAc/MeOH 95:5) to give Int. 1R2 tert-butyl 4-(4-(dimethylcarbamoyl)-phenyl)-4-fluoropiperidine-1-carboxylate. Int. 1R2 (0.55 g) was stirred 1 h in MeOH/4M HCl in dioxane (1:1, 20 mL), concentrated, and purified by SCX and HPLC to give Int. 1R1 4-(4-fluoropiperidin-4-yl)-N,N-dimethylbenzamide (62 mg).

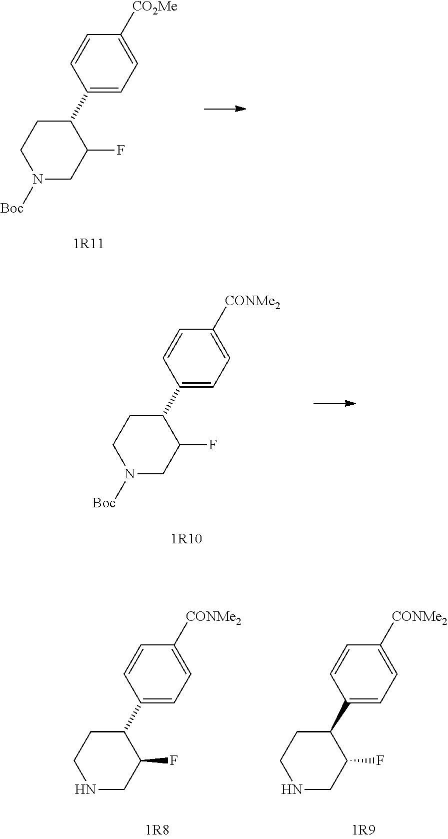

Int. 1AE14 (10.0 g) was stirred ON in THF/2.0 M BH₃-Me₂S in THF (11:6:1, 218 mL) at 0° C. to RT. 2.0 M aq. NaOH (38 mL) and 30% aq. H₂O₂ (5.9 mL) were added and stirring continued 1 h. The OL (sat. aq. Na₂S₂O₃/EtOAc) was washed with water and brine, dried, concentrated, and purified by FC (hexane/EtOAc 7:3) to give tert-butyl 3-hydroxy-4-(4-(methoxycarbonyl)-phenyl)piperidine-1-carboxylate (7.4 g). 7.0 g of this material was dissolved in DCM (140 mL) at −78° C. 50% Deoxo-Fluor in THF (4.5 mL) was added and stirring continued 6 h at −78° C. to RT. The OL (sat. aq. NaHCO₃/DCM) were washed with sat. aq. citric acid, dried, concentrated, and purified by FC (hexane/EtOAc 85:15) to give Int. 1R11 tert-butyl 3-fluoro-4-(4-(methoxycarbonyl)phenyl)-piperidine-1-carboxylate (4.8 g). Int. 1R11 (10.0 g) and LiOH—H₂O (6.1 g) were stirred ON in MeOH/THF/water (2:4:1, 230 mL) at 0° C. to RT and concentrated. The OL (aq. citric acid/10% MeOH in DCM) was dried and concentrated to give 4-(1-(tert-butoxy-carbonyl)-3-fluoropiperidin-4-yl)benzoic acid (8.0 g). 22 g of this material, HATU (39 g), and HN(CH₃)₂ (16.5 g, HCl salt) were stirred ON in DMF/DIPEA (6:7:1, 0.46 L) at 0° C. to RT. The OL (water/MeOH/DCM (5:95)) was dried, concentrated, and purified by FC (hexane/EtOAc 2:3) to give tert-butyl 4-(4-(dimethylcarbamoyl)phenyl)-3-fluoropiperidine-1-carboxylate (20 g). This material was stirred ON in DCM/TFA (37:1, 513 mL) at 0° C. to RT ON and concentrated. The aq. layer (water/Et₂O) was basified with sat. aq. NaHCO₃ and extracted with MeOH/DCM (5:95). The OLs were dried and concentrated to give 4-(3-fluoropiperidin-4-yl)-N,N-dimethyl-benzamide (14.5 g). This material was resolved by SFC using a Chiralpak IG 250×25 5 μm column operated at 30° C. using an eluent of 70% CO₂ and 30% MeOH containing 0.5% HNEt₂ (100 g/min) and a back pressure of 100 bar to give Int. 1R8 (5.3 g, first peak) and Int. 1R9 (5.1 g, second peak) 4-((3R,4R)-3-fluoropiperidin-4-yl)-N,N-dimethylbenzamide and 4-((3S,4S)-3-fluoropiperidin-4-yl)-N,N-dimethylbenzamide. The absolute configurations of these compounds were not determined.

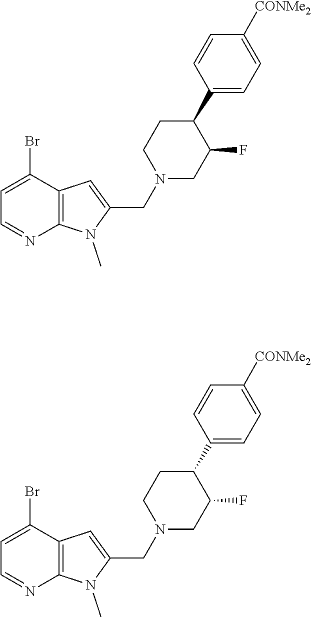

Int. 1F1 (0.10 g), DIPEA (0.21 mL), KI (20 mg), and Int. 1J1 were stirred ON in DMF (5 mL). The OL (EtOAc/water) was dried, concentrated, and purified by FC (hexane/EtOAc 3:7) to give Int. 1R12 N,N-Dimethyl-4-[(3R,4S)-1-[(4-bromo-1-methyl-pyrrolo[2,3-b]-pyridin-2-yl)methyl]-3-fluoro-4-piperidyl]benzamide (0.12 g). Int. 1R13 N,N-dimethyl-4-[(3S,4R)-1-[(4-bromo-1-methyl-pyrrolo[2,3-b]pyridin-2-yl)methyl]-3-fluoro-4-piperidyl]benzamide (0.12 g) was prepared similarly from Ints. 1F2/1J1 (0.10 g/0.16 g).

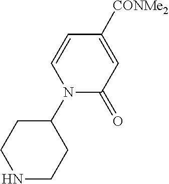

KOtBu (74 mg), K₂CO₃ (0.17 g), N,N-dimethyl-2-oxo-1H-pyridine-4-carboxamide (0.10 g), and tert-butyl 4-(p-tolylsulfonyloxy)piperidine-1-carboxylate (0.24 g) were stirred ON in DME (5 mL) at 110° C. ON. The OL (EtOAc/water) was dried, concentrated, and purified by FC (hexane to EtOAc) to give tert-butyl 4-[4-(dimethyl-carbamoyl)-2-oxo-1-pyridyl]piperidine-1-carboxylate (70 mg). tert-Butyl 4-[4-(dimethylcarbamoyl)-2-oxo-1-pyridyl]piperidine-1-carboxylate (0.20 g) was stirred in DCM/TFA (10:1, 5.5 mL) and concentrated to give Int. 1S2 N,N-Dimethyl-2-oxo-1-(4-piperidyl)pyridine-4-carbox-amide and Int. 1S2′ N,N-dimethyl-2-(4-piperidyloxy)pyridine-4-carboxamide (0.15 g, TFA salt).

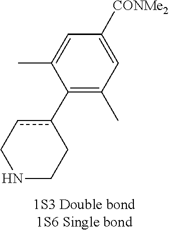

4-Bromo-N,N,3,5-tetramethylbenzamide (5.5 g), tert-butyl 4-(4,4,5,5-tetramethyl-1,3,2-dioxaborolan-2-yl)-3,6-dihydropyridine-1 (2H)-carboxylate (7.3 g), PdDPPFCl₂-DCM (0.88 g), and NaHCO₃ (5.4 g) were degassed in dioxane/water (3:1, 47 mL) and stirred ON at 100° C. The OL (EtOAc/water) was dried, concentrated, and purified by FC (pentane/EtOAc 1:1) to give tert-butyl 4-[4-(dimethylcarbamoyl)-2,6-dimethyl-phenyl]-3,6-dihydro-2H-pyridine-1-carboxylate (4.5 g). 0.3 g of this material was stirred ON in 2.9M HCl in dioxane (7 mL) at 0° C. to R, concentrated, and triturated in Et₂O. The solid was treated with sat. aq. NaHCO₃ and concentrated. The residue was dissolved in DCM, dried, and concentrated to give Int. 1S3 N,N,3,5-tetramethyl-4-(1,2,3,6-tetrahydro-pyridin-4-yl)benzamide (0.15 g). tert-Butyl 4-[4-(dimethylcarbamoyl)-2,6-dimethyl-phenyl]-3,6-dihydro-2H-pyridine-1-carboxylate (50 mg) was hydrogenated 48 h using PtO₂ (15 mg) in AcOH (3 mL), filtered, and concentrated to give Int. 1S6 N,N,3,5-tetramethyl-4-(piperidin-4-yl)benzamide (35 mg).

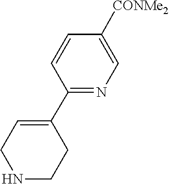

6-Bromo-N,N-dimethylnicotinamide 20.0 g), tert-butyl 4-(4,4,5,5-tetramethyl-1,3,2-dioxaborolan-2-yl)-3,6-dihydropyridine-1 (2H)-carboxylate (27.0 g), NaHCO₃ (25.7 g), and PdDPPFCl₂-DCM (0.71 g) were degassed in dioxane/water (4:1, 0.5 L), refluxed ON, and filtered. The OL (water/EtOAc) was dried, concentrated, and purified by FC (pentane/EtOAc 45:55) to give tert-butyl 4-[5-(dimethyl-carbamoyl)-2-pyridyl]-3,6-dihydro-2H-pyridine-1-carboxylate (17 g). 1.0 g of this material was stirred ON in DCM/4M HCl in dioxane (2.6:1, 14 mL) at 0° C. to RT. The mixture was combined with another two batches prepared on 0.1 g scale and concentrated. The residue was stirred in water at pH 8 (adjusted with NaHCO₃). The mixture was purified by FC (reverse-phase C18 column; 8% ACN in 0.001% aq. formic acid) to give Int. 1S4 N,N-dimethyl-1′,2′,3′,6′-tetrahydro-[2,4′-bipyridine]-5-carboxamide (0.60 g).

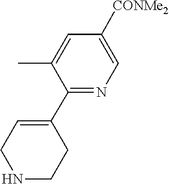

6-Bromo-N,N,5-trimethyl-pyridine-3-carboxamide (3.2 g), NaHCO₃ (3.9 g), PdDPPFCl₂-DCM (0.27 g), and tert-butyl 4-(4,4,5,5-tetramethyl-1,3,2-dioxaborolan-2-yl)-3,6-dihydropyridine-1 (2H)-carboxylate (6.1 g) were degassed in dioxane/water (4:1, 13 mL) and stirred ON at 110° C. The mixture was filtered, concentrated, and purified by FC (hexane/EtOAc 1:4) to give tert-butyl 4-[5-(dimethyl-carba-moyl)-3-methyl-2-pyridyl]-3,6-dihydro-2H-pyridine-1-carboxylate (4.0 g). 0.35 g of this material was stirred ON in DCM/TFA (10:1, 9 mL) at 0° C. to RT, concentrated, and triturated in Et₂O/pentane to give Int. 1S5 N,N,3-trimethyl-1′,2′,3′,6′-tetrahydro-[2,4′-bipyridine]-5-carboxamide (0.35 g, TFA salt).

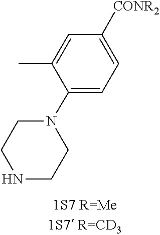

tert-Butyl piperazine-1-carboxylate (5.0 g), 4-bromo-N,N,3-trimethylbenzamide (7.1 g), Pd₂dba₃ (1.19 g), XPhos (1.29 g), and NaOtBu (5.19 g) were degassed in dioxane (50 mL) and stirred ON at 100° C. and filtered. The OL (water/EtOAc) was dried, concentrated, and purified by FC (pentane/EtOAc 3:7) to give tert-butyl 4-[4-(dimethyl-carbamoyl)-2-methyl-phenyl]piperazine-1-carboxylate (6.1 g). 7.2 g of this material was stirred ON in 4M HCl in dioxane/DCM (1.8:1, 55 mL) at 0° C. to RT, concentrated, and triturated in Et₂O/pentane to give Int. 1S7 N,N,3-trimethyl-4-(piperazin-1-yl)benzamide (5.0 g, HCl salt). Int. 1S7′ 3-methyl-N,N-bis(methyl-d₃)-4-(piperazin-1-yl)benzamide was prepared similarly from 4-bromo-3-methyl-N,N-bis(methyl-d₃)benzamide.

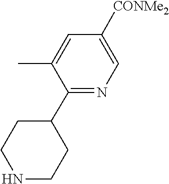

6-Bromo-N,N,5-trimethyl-pyridine-3-carboxamide (3.2 g), NaHCO₃ (3.9 g), and PdDPPFCl₂-DCM (0.27 g), tert-butyl 4-(4,4,5,5-tetramethyl-1,3,2-dioxaborolan-2-yl)-3,6-dihydropyridine-1 (2H)-carboxylate (6.1 g) were degassed in dioxane/water (4:1, 14 mL) and stirred ON at 110° C. The mixture was filtered, concentrated, and purified by FC (hexane/EtOAc 1:4) to give tert-butyl 4-[5-(dimethylcarba-moyl)-3-methyl-2-pyridyl]-3,6-dihydro-2H-pyridine-1-carboxylate (4.0 g). 4.2 g of this material was hydrogenated ON using 10% Pd/C (2.0 g) in MeOH (100 mL), filtered, concentrated, and purified by FC (hexane/EtOAc 1:9) to give tert-butyl 4-[5-(dimethyl-carbamoyl)-3-methyl-2-pyridyl]piperidine-1-carboxylate (3.5 g). 4.3 g of this material was stirred ON in DCM/TFA (1.5:1, 25 mL) at 0° C. to RT, concentrated, and triturated in pentane/Et₂O to give Int. 1S8 N,N,5-Trimethyl-6-(piperidin-4-yl)nicotinamide (5.0 g, TFA salt).

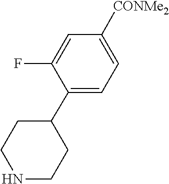

4-Bromo-3-fluoro-N,N-dimethyl-benzamide (1.0 g), tert-butyl 4-(4,4,5,5-tetramethyl-1,3,2-dioxaborolan-2-yl)-3,6-dihydropyridine-1 (2H)-carboxylate (1.51 g), Na₂CO₃ (1.72 g), and PdDPPFCl₂-DCM (0.17 g) were degassed in dioxane/water (4:1, 10 mL) and stirred ON at 100° C. The OL (water/EtOAc) was washed with brine, dried, concentrated, and purified by FC (hexane to EtOAc) to give tert-butyl 4-[4-(dimethyl-carbamoyl)-2-fluoro-phenyl]-3,6-dihydro-2H-pyridine-1-carboxylate (1.20 g). This material was hydrogenated ON using 10% Pd/C (45 mg) in MeOH (2 mL), filtered, concentrated, stirred ON in 4M HCl in dioxane (10 mL), concentrated, and HPLC-purified to give Int. 1S18 3-fluoro-N,N-dimethyl-4-(piperidin-4-yl)benzamide (63 mg).

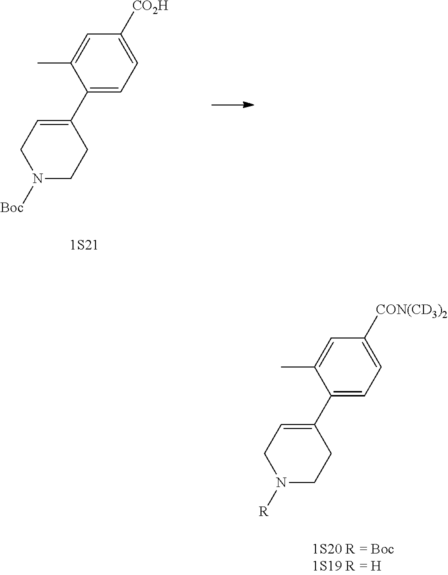

96% aq. H₂SO₄ (2.5 mL) was added to a solution of 4-bromo-3-methyl-benzoic acid (20 g) in MeOH (300 mL). The mixture was stirred at 90° C. ON and concentrated. The organic layer (sat. aq. NaHCO₃/EtOAc) was dried and concentrated to give methyl 4-bromo-3-methyl-benzoate (20 g). 10 g of this material, tert-butyl 4-(4,4,5,5-tetramethyl-1,3,2-dioxaborolan-2-yl)-3,6-dihydropyridine-1 (2H)-carboxylate (14.8 g), Na₂CO₃ (13.8 g), and PdDPPFCl₂-DCM (0.84 g) were degassed in dioxane/water (2.3:1, 100 mL), stirred ON at 90° C., filtered, concentrated, and purified by FC (hexane/EtOAc 95:5) to give tert-butyl 4-(4-methoxycarbonyl-2-methyl-phenyl)-3,6-dihydro-2H-pyridine-1-carboxylate (7.0 g). 2.5 g of this material and LiOH—H₂O (2.0 g) were stirred ON in EtOH/THF/water (2:1:1, 24 mL) at 0° C. to RT, concentrated, and triturated in dilute aq. citric acid to give Int. 1S21 4-(1-tert-butoxycarbonyl-3,6-dihydro-2H-pyridin-4-yl)-3-methyl-benzoic acid (2.0 g). Int. 1S20 (0.70 g) was prepared similarly to Int. 1D8 from Int. 1S21 (2.0 g). Int. 1S19 3-methyl-N,N-bis(methyl-d₃)-4-(1,2,3,6-tetrahydropyridin-4-yl)benzamide (0.31 g) was prepared similarly to Int. 1S7 from Int. 1S20 (0.80 g).

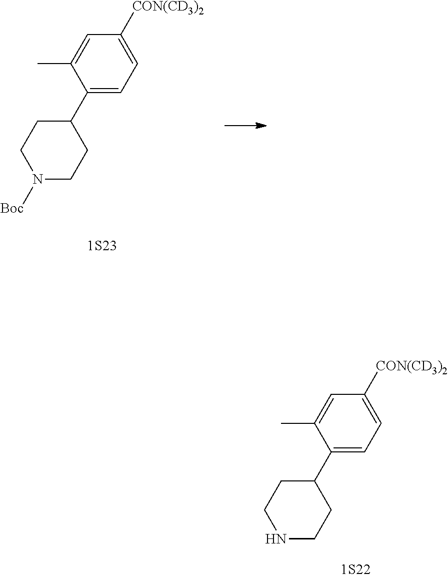

Int. 1S20 (0.35 g) was hydrogenated ON using 10% Pd/C (50 mg) in MeOH (10 mL), filtered, and concentrated to give Int. 1S23 (0.21 g). Int. 1S22 3-methyl-N,N-bis-(methyl-d₃)-4-(piperidin-4-yl)benzamide (0.45 g) was prepared similarly to Int. 1S7 from Int. 1S23 (0.90 g).

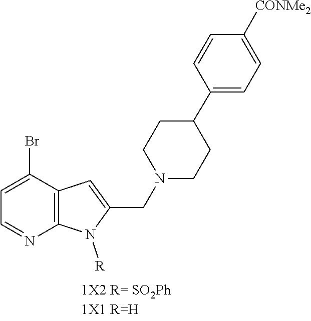

Int. 1A6 (0.30 g), N,N-dimethyl-4-(4-piperidyl)benzamide (0.34 g), and 4 Å MS were stirred 0.3 h in DCM (5 mL). STAB (0.52 g) was added and stirring was continued ON. The mixture was filtered, concentrated, and purified by FC (heptane to EtOAc/MeOH 1:4) to give Int. 1X2 4-[1-[[1-(benzenesulfonyl)-4-bromo-pyrrolo-[2,3-b]-pyridin-2-yl]-methyl]-4-piperidyl]-N,N-dimethyl-benzamide (0.22 g). Int. 1X2 (0.12 g) was stirred 2.5 h in EtOH/4M aq. NaOH (2.5:1, 2.8 mL) at 80° C. and concentrated. The OL (water/DCM) was concentrated to give Int. 1X1 4-[1-[(4-bromo-1H-pyrrolo[2,3-b]pyridin-2-yl)methyl]-4-piperidyl]-N,N-dimethyl-benzamide (94 mg).

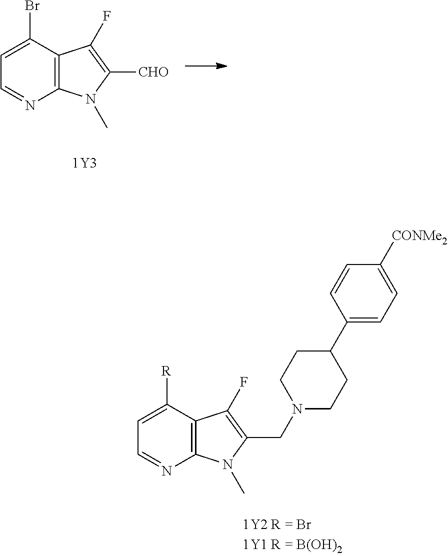

4-Bromo-3-fluoro-1-methyl-pyrrolo[2,3-b]pyridine (1.9 g) was stirred 2 h in THF/2.0 M LDA in THF (5:1, 24 mL) at −78° C. DMF (1.6 mL in THF (5 mL)) was added and stirring continued 2 h at −78° C. to RT. The OL (water/DCM) was washed with brine, dried, concentrated, and purified by FC (pentane/EtOAc 1:4) to give Int. 1Y3 4-bromo-3-fluoro-1-methyl-pyrrolo[2,3-b]pyridine-2-carbaldehyde (0.4 g). Int. 1Y3 (0.39 g) and N,N-dimethyl-4-(4-piperidyl)benzamide trifluoro acetate (0.38 g) were stirred ON in DCE/DIPEA (3.8:1, 6.3 mL) at 0° C. to RT. STAB (0.64 g) was added and stirring continued ON at 0° C. to RT. The OL (EtOAc/water) was washed brine, dried, concentrated, and purified by FC (pentane to EtOAc) to give Int. 1Y2 4-[1-[(4-bromo-3-fluoro-1-methyl-pyrrolo[2,3-b]pyridin-2-yl)methyl]-4-piperidyl]-N,N-dimethyl-benzamide (0.38 g). Int. 1Y2 (0.25 g), B₂Pin₂ (0.17 g), KOAc (0.16 g), and PdDPPFCl₂-DCM (37 mg) were degassed in THF (20 mL) and ON at 100° C., concentrated, and triturated in pentane to give Int. 1Y1 (2-((4-(4-(dimethylcarbamoyl)phenyl)-piperidin-1-yl)methyl)-3-fluoro-1-methyl-1H-pyrrolo[2,3-b]pyridin-4-yl)boronic acid (0.2 g).

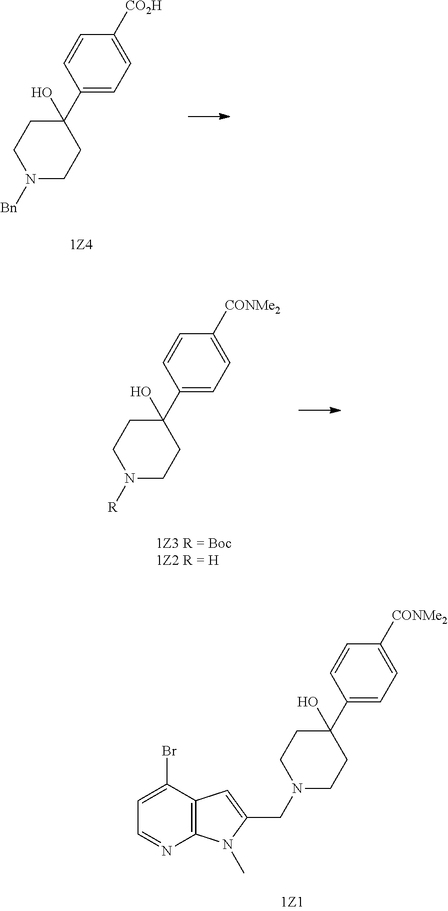

Int. 1R⁵ (0.5 g) and LiOH (181 mg) were stirred ON in MeOH/THF/water (3:5:1, 9 mL) and concentrated to give Int. 1Z4 4-(1-benzyl-4-hydroxypiperidin-4-yl)benzoic acid (0.40 g). Int. 1Z4 (0.40 g), HN(CH₃)₂ (0.31 g, HCl salt), and HATU (636 mg) were stirred ON in DMF/DIPEA (7:3:1, 9.1 mL). The OL (EtOAc/water) was concentrated, and purified by FC (heptane/EtOAc 5:3 to 3:7) to give Int. 1Z3 4-(1-benzyl-4-hydroxy-piperidin-4-yl)-N,N-dimethyl-benzamide. Int. 1Z3 (1.0 g) was hydrogenated 32 h using 10% Pd/C (0.5 g) in MeOH (20 mL), filtered, and concentrated to give Int. 1Z2 4-(4-hydroxypiperidin-4-yl)-N,N-dimethylbenzamide. Int. 1Z2 (0.20 g) and 4-bromo-1H-pyrrolo[2,3-b]pyridine-2-carboxaldehyde (0.19 g) were stirred 4 h in DCE/DIPEA (10:1, 3.3 mL). STAB (0.51 g) was added and stirring continued 32 h. The OL (water/DCM) was concentrated and purified by FC (hexane/EtOAc 1:0 to 4:1) to give Int. 1Z1 4-[1-[(4-Bromo-1-methyl-pyrrolo[2,3-b]pyridin-2-yl)methyl]-4-hydroxy-4-piperidyl]-N,N-dimethyl-benzamide (0.15 g).

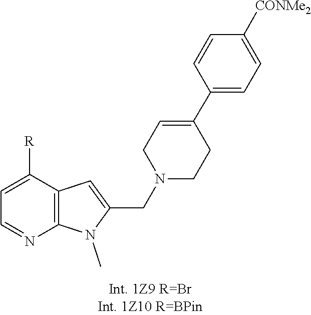

Int. 1J1 (0.97 g), KI (12 mg), Int. 1AE7 (1 g), and K₂CO₃ (1.6 g) were stirred 11 h in DMF (10 mL). The OL (water/MTBE) was concentrated to give Int. 1Z9 4-(1-((4-bromo-1-methyl-1H-pyrrolo[2,3-b]pyridin-2-yl)methyl)-1,2,3,6-tetrahydropyridin-4-yl)-N,N-dimethylbenzamide (0.67 g). Int. 1Z9 (0.67 g), KOAc (0.44 g), PdDPPFCl₂-DCM (24 mg), and bis(pinacolato)diboron (0.75 g) were stirred 48 h in dioxane (10 mL) at 90° C. under an inert atmosphere. The OL (water/MTBE) was concentrated to give Int. 1Z10 N,N-dimethyl-4-(1-((1-methyl-4-(4,4,5,5-tetramethyl-1,3,2-dioxaborolan-2-yl)-1H-pyrrolo-[2,3-b]pyridin-2-yl)methyl)-1,2,3,6-tetrahydropyridin-4-yl)benzamide (0.72 g). 6-bromopyridazin-3-amine (0.25 g) and di-tert-butyl dicarbonate (0.38 g) were stirred ON in 1M LiHMDs in THF (1.9 mL) and THF (5 mL) at 0° C. to RT under an inert atmosphere. The OL (sat. aq. NH₄Cl/EtOAc) was dried, and concentrated to give Int. 1Z11 tert-butyl (6-bromopyridazin-3-yl)carbamate (0.15 g).

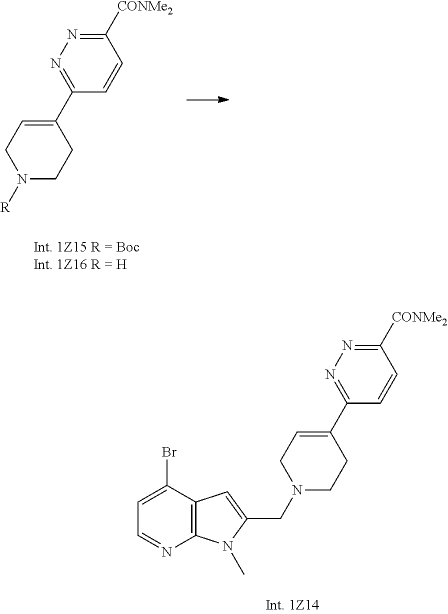

6-Chloro-N,N-dimethylpyridazine-3-carboxamide (0.85 g), tert-butyl 4-(4,4,5,5-tetramethyl-1,3,2-dioxaborolan-2-yl)-3,6-dihydropyridine-1 (2H)-carboxylate (1.6 g), K₂CO₃ (2.5 g), and PdDPPFCl₂-DCM (0.19 g) were stirred ON in dioxane/H₂O (4:1, 25 mL) at 100° C. under an inert atmosphere. The OL (water/EtOAc) was dried, concentrated, and purified by FC (CHCl₃/MTBE 1:0 to 0:1) to give Int. 1Z15 tert-butyl 4-(6-(dimethylcarbamoyl)pyridazin-3-yl)-3,6-dihydropyridine-1 (2H)-carboxylate (1 g). Int. 1Z15 (1 g) was stirred 1 h in 1,1,1,3,3,3-hexafluoro-2-propanol/10M HCl (19:1, 10.5 mL). The mixture was diluted with MTBE to precipitate Int. 1Z16 N,N-dimethyl-6-(1,2,3,6-tetrahydro-pyridin-4-yl)pyridazine-3-carboxamide (0.8 g, HCl salt). Ints. 1J1/1Z16 (0.2 g/0.3 g) were stirred 12 h in ACN/Et₃N (48:1, 20.4 mL). The mixture was concentrated and HPLC-purified to give Int. 1Z14 6-(1-((4-bromo-1-methyl-1H-pyrrolo[2,3-b]pyridin-2-yl)methyl)-1,2,3,6-tetrahydropyridin-4-yl)-N,N-dimethylpyridazine-3-carboxamide (70 mg).

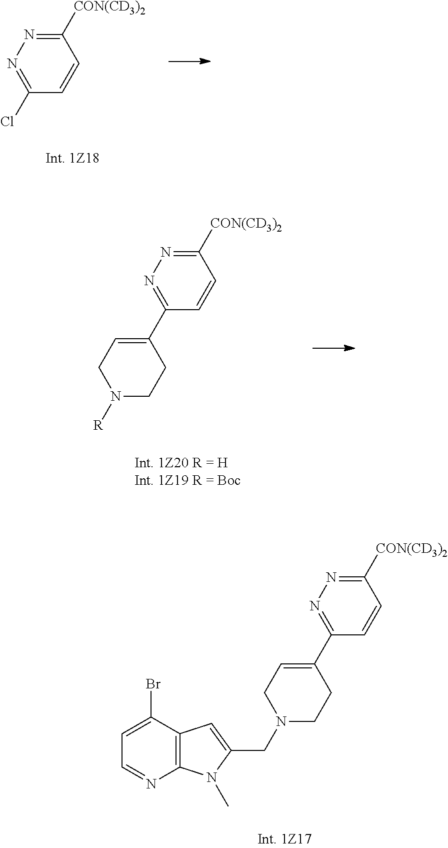

6-Chloropyridazine-3-carboxylic acid (1 g) was stirred in DCM/SOCl₂ (40:1, 20.5 mL) for 2 h at 40° C. and concentrated. Bis(methyl-d₃)amine hydrochloride (0.61 g) in DCM (20 mL) and Et₃N (2.6 mL) were added and the mixture was stirred ON. The mixture was concentrated and purified by FC (CHC13/ACN 1:0 to 1:1) to give Int. 1Z18 6-chloro-N,N-bis(methyl-d₃)pyridazine-3-carboxamide (1.2 g). Int. 1Z18 (1.2 g), tert-butyl 4-(4,4,5,5-tetramethyl-1,3,2-dioxaborolan-2-yl)-3,6-dihydropyridine-1 (2H)-carboxylate (2.1 g), K₂CO₃ (3.5 g), and PdDPPFCl₂-DCM (0.26 g) were stirred ON in dioxane/H₂O (4:1, 25 mL) at 100° C. under an inert atmosphere. The OL (water/EtOAc) was dried, concentrated, and purified by FC (CHCl₃/MTBE 1:0 to 0:1) to give Int. 1Z19 tert-butyl 4-(6-(bis-(methyl-d₃)carbamoyl)-pyridazin-3-yl)-3,6-dihydropyridine-1 (2H)-carboxylate (0.55 g). Int. 1Z19 (0.55 g) was stirred 1 h in 1,1,1,3,3,3-hexafluoro-2-propanol/10M HCl (26:1, 5.2 mL). The mixture was diluted with MTBE to precipitate Int. 1Z20 N,N-bis(methyl-d₃)-6-(1,2,3,6-tetrahydropyridin-4-yl)-pyridazine-3-carboxamide (0.2 g, HCl salt). Ints. 1J1/1Z20 (0.2 g/0.2 g) were stirred 12 h in ACN/Et₃N (48:1, 20.4 mL). The mixture was concentrated and HPLC-purified to give Int. 1Z17 6-(1-((4-bromo-1-methyl-1H-pyrrolo[2,3-b]-pyridin-2-yl)methyl)-1,2,3,6-tetrahydropyridin-4-yl)-N,N-bis(methyl-d₃)pyridazine-3-carboxamide (50 mg).

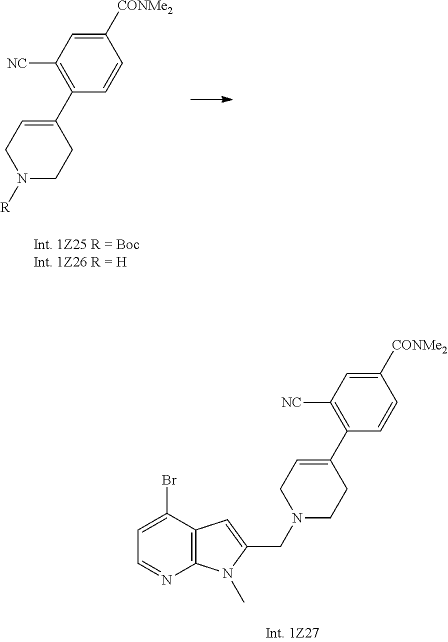

4-Chloro-3-cyanobenzoic acid (3.4 g) and di(1H-imidazol-1-yl)methanone (3.0 g) were refluxed 0.3 h in dioxane (50 mL). Dimethylamine hydrochloride (1.5 g) was added and stirring continued 10 h. The mixture was concentrated. The OL (aq. NaHSO₄/CHCl₃) was concentrated to give Int. 1Z24 4-chloro-3-cyano-N,N-dimethyl-benzamide (0.87 g). Int. 1Z24 (1 g), tert-butyl 4-(4,4,5,5-tetramethyl-1,3,2-dioxaborolan-2-yl)-3,6-dihydropyridine-1 (2H)-carboxylate (1.6 g), K₂CO₃ (2.0 g), and PdDPPFCl₂-DCM (0.20 g) were stirred in dioxane/H₂O (4:1, 20 mL) ON at 100° C. under an inert atmosphere. The mixture was filtered, concentrated, and purified by FC (CHCl₃/ACN 1:0 to 4:1) to give Int. 1Z25 tert-butyl 4-(2-cyano-4-(dimethyl-carbamoyl)phenyl)-3,6-dihydropyridine-1 (2H)-carboxylate (0.44 g). Int. 1Z25 (0.44 g) was stirred ON in 2.8M HCl in dioxane (10 mL). The mixture was concentrated to give Int. 1Z26 3-cyano-N,N-dimethyl-4-(1,2,3,6-tetrahydropyridin-4-yl)benzamide (0.3 g, HCl salt). Ints. 1J1/1Z26 (0.3 g/0.3 g) and K₂CO₃ (0.5 g) were stirred 1 1 h in DMF (10 mL). The mixture was diluted with water to precipitate Int. 1Z27 4-(1-((4-bromo-1-methyl-1H-pyrrolo[2,3-b]pyridin-2-yl)methyl)-1,2,3,6-tetrahydropyridin-4-yl)-3-cyano-N,N-dimethylbenzamide (0.12 g).

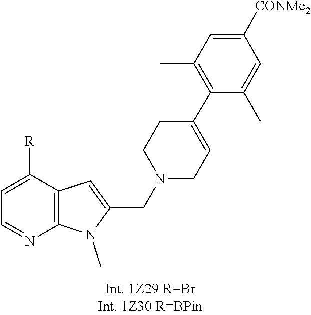

Ints. 1J1/RR44 (0.3 g/0.3 g) were stirred 12 h in ACN/Et₃N (29:2, 10.7 mL). The mixture was concentrated and HPLC-purified to give Int. 1Z29 4-(1-((4-bromo-1-methyl-1H-pyrrolo[2,3-b]pyridin-2-yl)methyl)-1,2,3,6-tetrahydropyridin-4-yl)-N,N,3,5-tetramethylbenzamide (0.3 g). Int. 1Z29 (0.3 g), bis(pinacolato)diboron (0.4 g), KOAc (0.5 g), and PdDPPFCl₂-DCM (50 mg) were stirred 12 h in dioxane (10 mL) at 80° C. under an inert atmosphere. The OL (water/EtOAc) was concentrated to give Int. 1Z30 N,N,3,5-tetramethyl-4-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)benzamide (0.3 g).

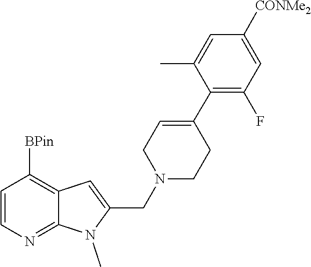

Int. 1Z50 (0.75 g), bis(pinacolato)diboron (0.78 g), PdDPPFCl₂-DCM (0.13 g), and KOAc (0.45 g) were stirred 1.3 h in dioxane (10 mL) at 110° C. under an inert atmosphere under MW conditions. The mixture was filtered through celite and concentrated to give Int. 1Z31 3-fluoro-N,N,5-trimethyl-4-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)benzamide (0.85 g).

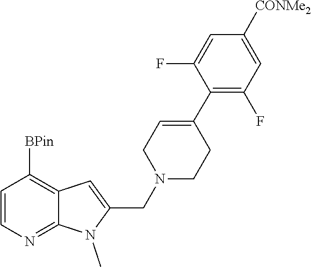

Int. 1Z65 (0.50 g), bis(pinacolato)diboron (0.52 g), PdDPPFCl₂-DCM (83 mg), and KOAc (0.30 g) were stirred 1 h in dioxane (10 mL) at 110° C. under an inert atmosphere under MW conditions. The mixture was filtered through celite, concentrated, and washed with pentane to give Int. 1Z33 3,5-difluoro-N,N-dimethyl-4-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)benzamide (0.50 g).

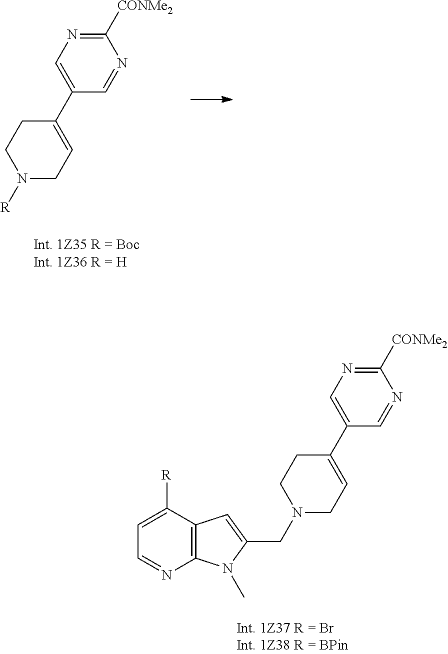

5-Bromo-N,N-dimethylpyrimidine-2-carboxamide (1.3 g), tert-butyl 4-(4,4,5,5-tetramethyl-1,3,2-dioxaborolan-2-yl)-3,6-dihydropyridine-1 (2H)-carboxylate (1.9 g), K₂CO₃ (2.3 g), and PdDPPFCl₂-DCM (0.22 g) were stirred ON in dioxane/H₂O (4:1, 20 mL) at 100° C. under an inert atmosphere. The mixture was concentrated and purified by FC (MTBE/MeOH) to give Int. 1Z35 tert-butyl 4-(2-(dimethylcarbamoyl)pyrimidin-5-yl)-3,6-dihydropyridine-1 (2H)-carboxylate (0.9 g). Int. 1Z35 (0.9 g) was stirred ON in 2.8M HCl in dioxane (10 mL). The mixture was concentrated to give Int. 1Z36 N,N-dimethyl-5-(1,2,3,6-tetrahydropyridin-4-yl)pyrimidine-2-carboxamide (0.65 g, HCl salt). Ints. 1J1/1Z36 (0.3 g/0.3 g) and K₂CO₃ (0.5 g) were stirred 11 h in DMF (10 mL). The mixture was diluted with water to precipitate Int. 1Z37 5-(1-((4-bromo-1-methyl-1H-pyrrolo[2,3-b]pyridin-2-yl)methyl)-1,2,3,6-tetrahydropyridin-4-yl)-N,N-dimethylpyrimidine-2-carboxamide (0.35 g). Int. 1Z37 (0.35 g), bis(pinacolato)diboron (0.70 g), PdDPPFCl₂-DCM (10 mg), and KOAc (0.40 g) were stirred 14 h in dioxane (10 mL) at 90° C. under an inert atmosphere. The OL (water/chloroform) was concentrated and HPLC-purified to give Int. 1Z38 N,N-dimethyl-5-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)pyrimidine-2-carboxamide (90 mg).

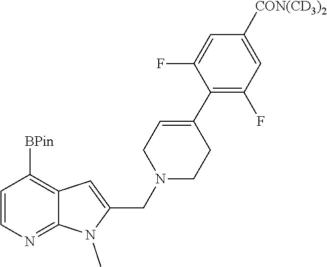

Int. 1Z65 (0.50 g), bis(pinacolato)diboron (0.52 g), PdDPPFCl₂-DCM (82 mg), and KOAc (0.30 g) were stirred 1 h in dioxane (6 mL) at 120° C. under MW conditions. The mixture was filtered through celite and concentrated to give Int. 1Z40 3,5-difluoro-N,N-bis(methyl-d₃)-4-(1-((1-methyl-4-(4,4,5,5-tetra-methyl-1,3,2-dioxaborolan-2-yl)-1H-pyrrolo[2,3-b]pyridin-2-yl)methyl)-1,2,3,6-tetrahydropyridin-4-yl)benzamide (0.60 g).

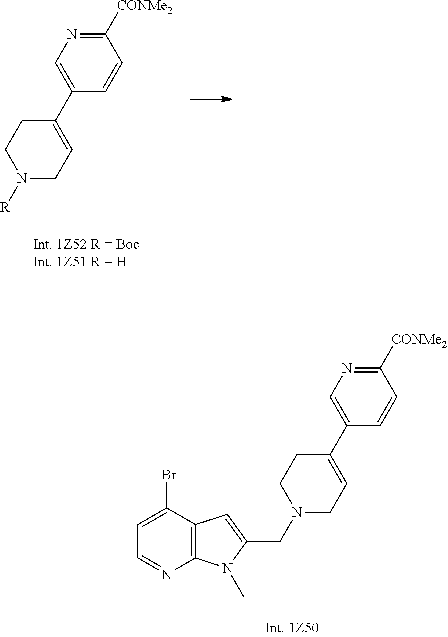

5-Bromo-N,N-dimethylpicolinamide (1.5 g), tert-butyl 4-(4,4,5,5-tetramethyl-1,3,2-dioxaborolan-2-yl)-3,6-dihydropyridine-1 (2H)-carboxylate (2.2 g), K₂CO₃ (2.7 g), and PdDPPFCl₂-DCM (0.27 g) were stirred ON in dioxane/H₂O (4:1, 20 mL) ON 100° C. under an inert atmosphere. The mixture was concentrated and purified by FC (hexane/MTBE 1:0 to 0:1) to give Int. 1Z52 tert-butyl 6-(dimethyl-carbamoyl)-3′,6′-dihydro-[3,4′-bipyridine]-1′(2′H)-carboxylate (1 g). Int. 1Z51 (1 g) was stirred ON in 2.8M HCl in dioxane (10 mL). The mixture was concentrated to give Int. 1Z52 N,N-dimethyl-1′,2′,3′,6′-tetrahydro-[3,4′-bipyridine]-6-carboxamide (0.5 g, HCl salt). Ints. 1J1/1Z52 (0.6 g/0.5 g) and K₂CO₃ (0.9 g) were stirred 11 h in DMF (10 mL). The mixture was diluted with water to precipitate Int. 1Z50 1′-((4-bromo-1-methyl-1H-pyrrolo[2,3-b]pyridin-2-yl)methyl)-N,N-dimethyl-1′,2′,3′,6′-tetrahydro-[3,4′-bipyridine]-6-carboxamide (0.4 g).

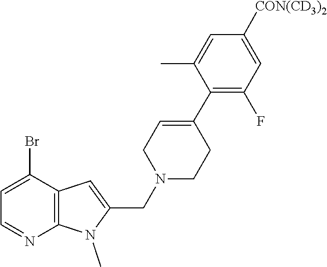

Int. 1AE9 (0.45 g), bis(pinacolato)diboron (0.35 g), PdDPPFCl₂-DCM (75 mg), and KOAc (0.27 g) were stirred 1 h in dioxane (5 mL) for 1 h at 110° C. under MW conditions. The mixture was filtered through celite and concentrated to give Int. 1Z53 3-fluoro-5-methyl-N,N-bis(methyl-d₃)-4-(1-((1-methyl-4-(4,4,5,5-tetramethyl-1,3,2-dioxaborolan-2-yl)-1H-pyrrolo[2,3-b]pyridin-2-yl)methyl)-1.2,3.6-tetrahydropyridin-4-yl)benzamide (0.40 g).

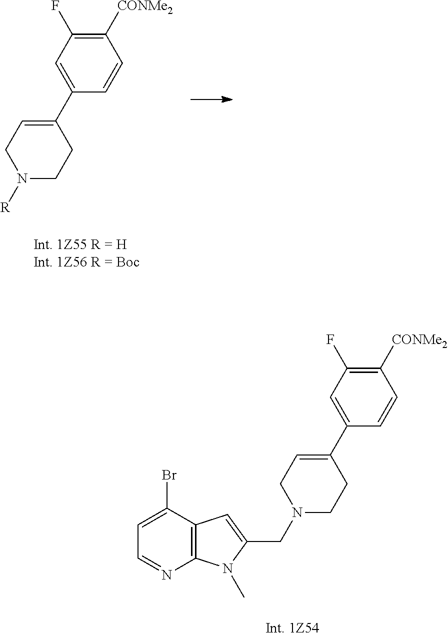

PdDPPFCl₂-DCM (0.25 g), 4-bromo-2-fluoro-N,N-dimethylbenzamide (1.5 g), tert-butyl 4-(4,4,5,5-tetramethyl-1,3,2-dioxaborolan-2-yl)-3,6-dihydropyridine-1 (2H)-carboxylate (2.1 g), and K₂CO₃ (2.5 g) were stirred ON in dioxane/H₂O (4:1, 20 mL) at 100° C. under an inert atmosphere. The mixture was concentrated and purified by FC (hexane/MTBE 3:2 to 0:1) to give Int. 1Z55 tert-butyl 4-(4-(dimethyl-carbamoyl)-3-fluorophenyl)-3,6-dihydropyridine-1 (2H)-carboxylate (1 g). Int. 1Z55 (1 g) was stirred ON in 2.8M HCl in dioxane (10 mL). The mixture was concentrated to give Int. 1Z56 2-fluoro-N,N-dimethyl-4-(1,2,3,6-tetrahydropyridin-4-yl)benzamide (0.5 g, HCl salt). Ints. 1J1/PP60 (0.5 g/0.5 g) and K₂CO₃ (0.8 g) were stirred 11 h in DMF (10 mL). The mixture was diluted with water to precipitate Int. 1Z54 4-(1-((4-bromo-1-methyl-1H-pyrrolo[2,3-b]pyridin-2-yl)methyl)-1,2,3,6-tetra-hydropyridin-4-yl)-2-fluoro-N,N-dimethylbenzamide (0.4 g).

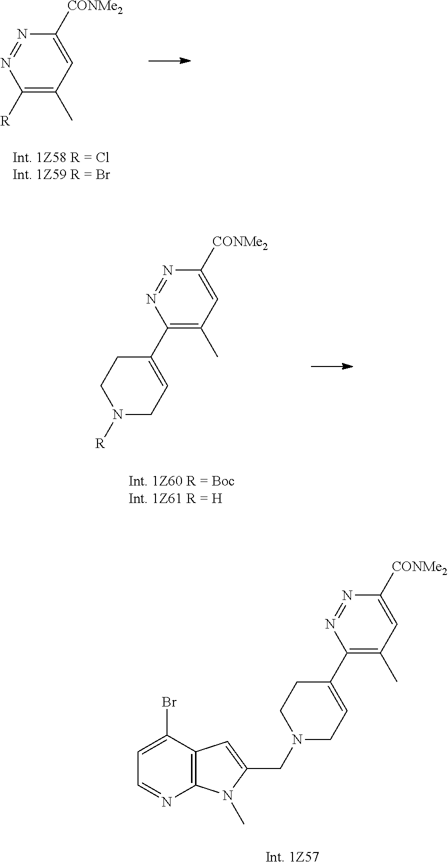

CDI (2.6 g) in THF (175 mL) was added dropwise to a mixture of 6-chloro-5-methylpyridazine-3-carboxylic acid (2 g) in THF (175 mL). The mixture was refluxed 3.5 h. Dimethylamine hydrochloride (2.8 g) and Et₃N (5.2 mL) were added and stirring continued 16 h at RT. The OL (brine/EtOAc) was concentrated to give Int. 1Z58 6-chloro-N,N,5-trimethylpyridazine-3-carboxamide (0.8 g). Int. 1Z58 was refluxed ON in bromotrimethylsilane (10 mL). The mixture was concentrated and purified by preparative TLC (hexane/EtOAc, 3:1) to give Int. 1Z59 6-bromo-N,N,5-trimethylpyridazine-3-carbox-amide (0.9 g). PdDPPFCl₂-DCM (0.3 g), Int. 1Z59 (0.9 g), tert-butyl 4-(4,4,5,5-tetramethyl-1,3,2-dioxa-borolan-2-yl)-3,6-dihydropyridine-1 (2H)-carboxylate (1.0 g), and K₂CO₃ (2.0 g) were refluxed ON in dioxane/H₂O (15:4, 190 mL) under an inert atmosphere. The OL (aq. NaHCO₃/EtOAc) was dried, and concentrated to give Int. 1Z60 tert-butyl 4-(6-(dimethylcarbamoyl)-4-methylpyridazin-3-yl)-3,6-dihydropyridine-1 (2H)-carboxylate (1.3 g). Int. 1Z60 (1.3 g) was stirred ON in 2.8M HCl in dioxane (10 mL). The residue after concentration was triturated in EtOAc to give Int. 1Z61 N,N,5-trimethyl-6-(1,2,3,6-tetrahydropyridin-4-yl)pyridazine-3-carboxamide (0.9, HCl salt). STAB (0.30 g) and Ints. 1AB8/1Z61 (0.1 g/0.1 g) were stirred ON in DCE/Et₃N (73:1, 10.1 mL) at 0° C. to RT. The residue after concentration was dissolved in THF, filtered through silica, and concentrated to give Int. 1Z57 6-(1-((4-bromo-1-methyl-1H-pyrrolo[2,3-b]pyridin-2-yl)methyl)-1,2,3,6-tetrahydropyridin-4-yl)-N,N,5-trimethylpyridazine-3-carboxamide (0.19 g).

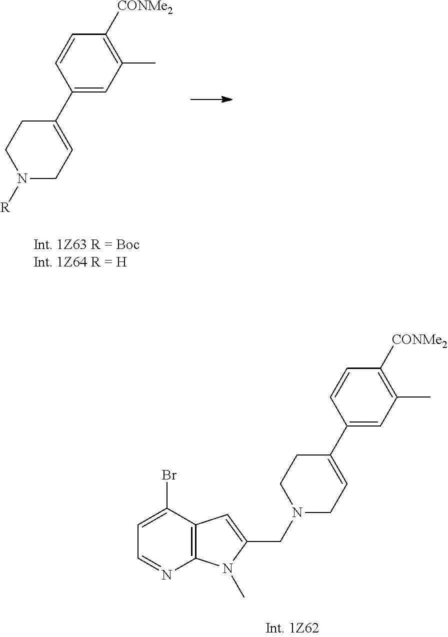

PdDPPFCl₂-DCM (1.1 g), 4-bromo-N,N,2-trimethylbenzamide (3.3 g), K₂CO₃ (5.7 g), and tert-butyl 4-(4,4,5,5-tetramethyl-1,3,2-dioxaborolan-2-yl)-3,6-dihydropyridine-1 (2H)-carboxylate (5.9 g) were refluxed ON in dioxane/H₂O (15:4, 190 mL) under an inert atmosphere. The OL (aq. NaHCO₃/EtOAc) was dried and concentrated to give Int. 1Z63 tert-butyl 4-(4-(dimethylcarbamoyl)-3-methyl-phenyl)-3,6-dihydropyridine-1 (2H)-carboxylate (4.1 g). Int. 1Z63 (4.1 g) was stirred ON in 2.8M HCl in dioxane (30 mL). The residue after concentration was triturated in EtOAc to give Int. 1Z64 N,N,2-trimethyl-4-(1,2,3,6-tetrahydropyridin-4-yl)benzamide (2.3 g, HCl salt). STAB (0.30 g) and Ints. 1AB8/1Z64 (0.1 g/0.1 g) were stirred ON in DCE/Et₃N (73:1, 10.1 mL) at 0° C. to RT. The residue after concentration was dissolved in THF, filtered through silica, and concentrated to give Int. 1Z62 4-(1-((4-bromo-1-methyl-1H-pyrrolo[2,3-b]pyridin-2-yl)methyl)-1,2,3,6-tetrahydropyridin-4-yl)-N,N,2-trimethylbenzamide (0.19 g).

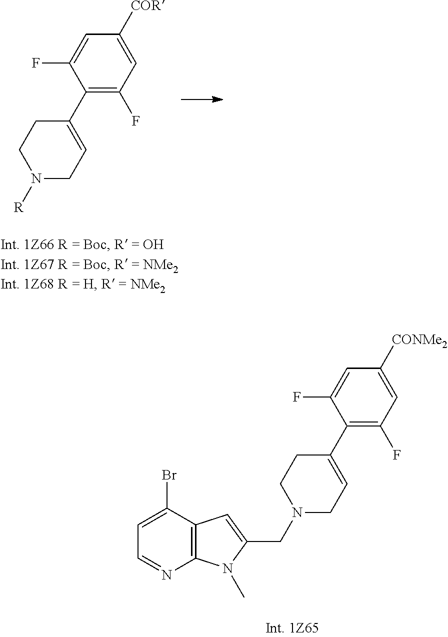

LiOH·H₂O (9.5 g) and Int. 3C18 (16 g) were stirred 16 h in THF/MeOH/H₂O (2:1:1, 300 mL) at 0° C. to RT. The residue after concentration was stirred in aq. citric acid to precipitate Int. 1Z66 4-(1-(tert-butoxy-carbonyl)-1,2,3,6-tetrahydropyridin-4-yl)-3,5-difluorobenzoic acid (10 g). Int. 1Z66 (5 g), HATU (8.4 g), and dimethylamine hydrochloride (1.8 g) were stirred 16 h in DMF (40 mL) and DIPEA (13 mL) at 0° C. to RT. The OL (water/EtOAc) was dried, concentrated, and purified by FC (PE/EtOAc 2:1) to give Int. 1Z67 tert-butyl 4-(4-(dimethylcarbamoyl)-2,6-difluorophenyl)-3,6-dihydropyridine-1 (2H)-carboxylate (3.7 g). Int. 1Z67 (3.7 g) was stirred 16 h in 2M HCl in dioxane (60 mL) at 0° C. to RT. The mixture was concentrated to give Int. 1Z68 3,5-difluoro-N,N-dimethyl-4-(1,2,3,6-tetrahydropyridin-4-yl)benzamide (2.9 g, HCl salt). Ints. 1J1/1Z68 (0.35 g/0.49 g), KI (22 mg), and K₂CO₃ (0.56 g) were stirred 16 h in ACN (20 mL) at 0° C. to RT. The OL (water/EtOAc) was dried, concentrated, and purified by FC (PE/EtOAc 3:7) to give Int. 1Z65 4-(1-((4-bromo-1-methyl-1H-pyrrolo[2,3-b]pyridin-2-yl)methyl)-1,2,3,6-tetrahydropyridin-4-yl)-3,5-difluoro-N,N-dimethylbenzamide (0.35 g).

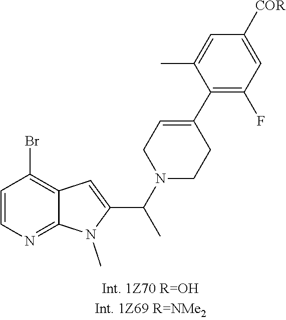

LiOH·H₂O (33 g) and Int. 1AF87 (76 g) were stirred 16 h in THF/MeOH/H₂O (2:1:1, 1.5 L) at 0° C. to RT. The residue after concentration was stirred in aq. citric acid at 0° C. to precipitate Int. 1Z70 4-(1-(1-(4-bromo-1-methyl-1H-pyrrolo[2,3-b]pyridin-2-yl)ethyl)-1,2,3,6-tetrahydropyridin-4-yl)-3-fluoro-5-methylbenzoic acid (68 g). Int. 1Z70 (70 g), dimethylamine hydrochloride (36 g), and HATU (84 g) were stirred 12 h in DMF (300 mL) and DIPEA (180 mL) at 0° C. to RT. The mixture was diluted with water to precipitate a solid that was dried to give Int. 1Z69 4-(1-(1-(4-bromo-1-methyl-1H-pyrrolo-[2,3-b]pyridin-2-yl)ethyl)-1,2,3,6-tetrahydropyridin-4-yl)-3-fluoro-N,N,5-trimethylbenzamide (63 g).

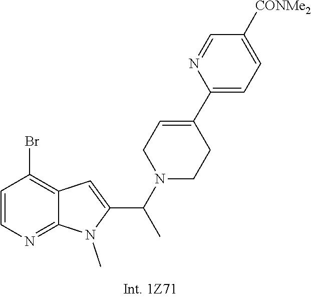

Ints. 1AF88/1S4 (1.5 g/1.5 g), KI (91 mg), and Cs₂CO₃ (8.9 g) were stirred 16 h in ACN (15 mL) at 0° C. to RT. The OL (water/EtOAc) was dried, concentrated, and purified by FC (DCM/MeOH 19:1) to give Int. 1Z71 1′-(1-(4-bromo-1-methyl-1H-pyrrolo[2,3-b]pyridin-2-yl)ethyl)-N,N-dimethyl-1′,2′,3′,6′-tetrahydro-[2,4′-bipyridine]-5-carboxamide (0.50 g).

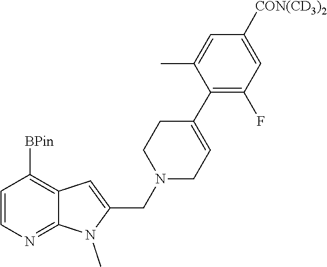

3-fluoro-5-methyl-N,N-bis(methyl-d₃)-4-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)benzamide

Int. 1AE9 (0.45 g), bis(pinacolato)diboron (0.35 g), PdDPPFCl₂-DCM (75 mg), and KOAc (0.27 g) were stirred 1 h in dioxane (5 mL) at 110° C. under MW conditions. The mixture was filtered through celite and concentrated to give Int. 1Z73 3-fluoro-5-methyl-N,N-bis(methyl-d₃)-4-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)benzamide (0.40 g).

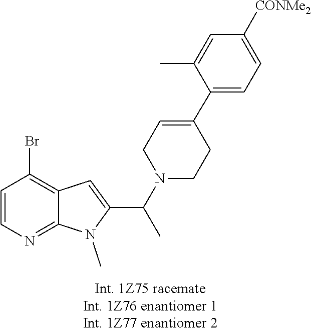

Int. 1AE38 (3.2 g) and Cs₂CO₃ (18 g) were stirred 0.3 h in ACN (20 mL). Int. 1AF88 (3 g) and KI (0.91 g) were added and stirring continued 12 h at 0° C. to RT. The OL (water/EtOAc) was dried, concentrated, and purified by FC (hexane/EtOAc 1:9) to give Int. 1Z75 4-(1-(1-(4-bromo-1-methyl-1H-pyrrolo[2,3-b]pyridin-2-yl)ethyl)-1,2,3,6-tetrahydropyridin-4-yl)-N,N,3-trimethylbenzamide (0.90 g). Int. 1Z75 (0.90 g) was resolved by SFC on a SFC-150-022 instrument fitted with a Chiral ART Amylose-C NEO 250×30 mm 5 μm column operated at 30° C. and an eluent of 80% CO₂ and 20% MeOH at a (100 g/min) and a back pressure of 100 bar to give Int. 1Z76 (0.15 g, first peak) and Int. 1Z77 (0.15 g, second peak) (S)-4-(1-(1-(4-bromo-1-methyl-1H-pyrrolo[2,3-b]pyridin-2-yl)ethyl)-1,2,3,6-tetrahydropyridin-4-yl)-N,N,3-trimethylbenzamide and (R)-4-(1-(1-(4-bromo-1-methyl-1H-pyrrolo[2,3-b]pyridin-2-yl)ethyl)-1,2,3,6-tetrahydropyridin-4-yl)-N,N,3-trimethylbenzamide. The absolute configurations of these compounds were not determined.

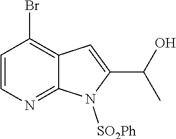

3M CH₃MgCl in THF (32 mL) was added slowly to a solution of Int. 1A6 (11.5 g) in THF (310 mL) at −78° C. The mixture was stirred 0.5 h at −78° C. to RT. The OL (aq. NH₄Cl/EtOAc) was dried, and concentrated to give Int. 1Z79 1-(4-bromo-1-(phenylsulfonyl)-1H-pyrrolo[2,3-b]pyridin-2-yl)ethan-1-ol (12 g).

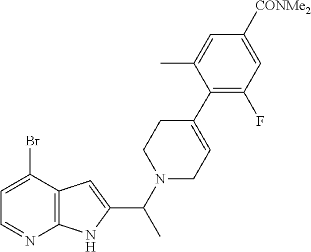

SOCl₂ (0.18 mL) was added to a solution of Int. 1Z79 (0.1 g) in toluene (3 mL). The mixture was stirred 0.5 h and concentrated. Int. 1AE12′ (90 mg), KI (20 mg), and Cs₂CO₃ (0.40 g) were stirred ON in a solution of the residue in ACN (5 mL). The mixture was HPLC-purified to give Int. 1Z80 4-(1-(1-(4-bromo-1H-pyrrolo[2,3-b]pyridin-2-yl)ethyl)-1,2,3,6-tetrahydropyridin-4-yl)-3-fluoro-N,N,5-trimethylbenzamide (13 mg).

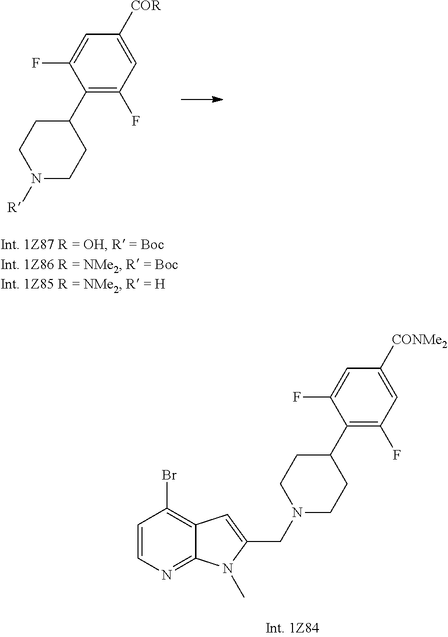

LiOH·H₂O (9.8 g) and Int. 3C17 (16.5 g) were stirred 16 h in THF/MeOH/H₂O (2:1:1, 600 mL) at 0° C. to RT. The residue after concentration was stirred in aq. citric acid, filtered, and dried to give Int. 1Z87 4-(1-(tert-butoxycarbonyl)piperidin-4-yl)-3,5-difluorobenzoic acid (11.5 g). HATU (8.3 g), Int. 1Z87 (5 g), and dimethylamine hydrochloride (1.8 g) were stirred 16 h in DMF (30 mL) and DIPEA (13 mL) 0° C. to RT. The OL (water/EtOAc) was dried, concentrated, and purified by FC (PE/EtOAc 2:1) to give Int. 1Z86 tert-butyl 4-(4-(dimethylcarbamoyl)-2,6-difluorophenyl)piperidine-1-carboxylate (3.2 g). Int. 1Z86 (3.2 g) was stirred 16 h in 2M HCl in dioxane (60 mL) at 0° C. to RT. The mixture was concentrated to give Int. 1Z85 3,5-difluoro-N,N-dimethyl-4-(piperidin-4-yl)benzamide (2.3 g, HCl salt). Ints. 1J1/1Z85 (0.35 g/0.36 g) and K₂CO₃ (0.93 g) were stirred 16 h in ACN (10 mL) at 0° C. to RT. The mixture was filtered, concentrated, and purified by FC (PE/EtOAc 1:4) to give Int. 1Z84 4-(1-((4-bromo-1-methyl-1H-pyrrolo[2,3-b]pyridin-2-yl)methyl)piperidin-4-yl)-3,5-difluoro-N,N-dimethyl-benzamide (0.40 g).

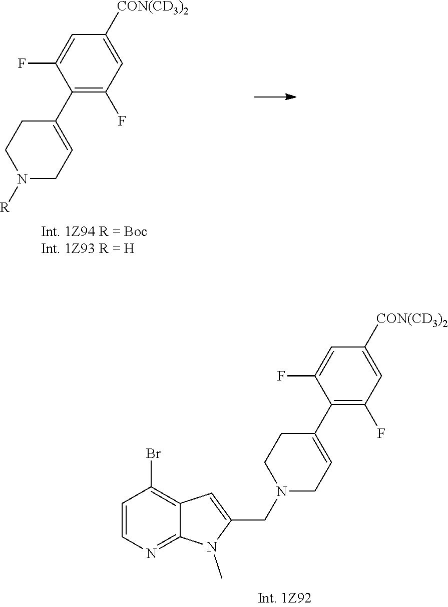

Int. 1Z66 (5 g), bis(methyl-d₃)amine hydrochloride (1.5 g), and HATU (8.4 g) were stirred 16 h in DMF (40 mL) and DIPEA (13 mL) at 0° C. to RT. The OL (water/EtOAc) was dried, concentrated, and purified by FC (PE/EtOAc 2:1) to give Int. 1Z94 tert-butyl 4-(4-(bis(methyl-d₃)carbamoyl)-2,6-difluoro-phenyl)-3,6-dihydropyridine-1 (2H)-carboxylate (3.5 g). Int. 1Z94 (3.6 g) was stirred 16 h in 2M HCl in dioxane (60 mL) at 0° C. to RT. The mixture was concentrated to give Int. 1Z93 3,5-difluoro-N,N-bis-(methyl-d₃)-4-(1,2,3,6-tetrahydropyridin-4-yl)benzamide (2.7 g, HCl salt). Ints. 1J1/1Z93 (0.40 g/0.57 g), KI (77 mg), and K₂CO₃ (0.64 g) were stirred 16 h in ACN (20 mL) at 0° C. to RT. The OL (water/EtOAc) was dried, concentrated and, purified by FC (PE/EtOAc 3:7) to give Int. 1Z92 4-(1-((4-bromo-1-methyl-1H-pyrrolo[2,3-b]pyridin-2-yl)methyl)-1,2,3,6-tetrahydropyridin-4-yl)-3,5-difluoro-N,N-bis(methyl-d₃)benzamide (0.41 g).

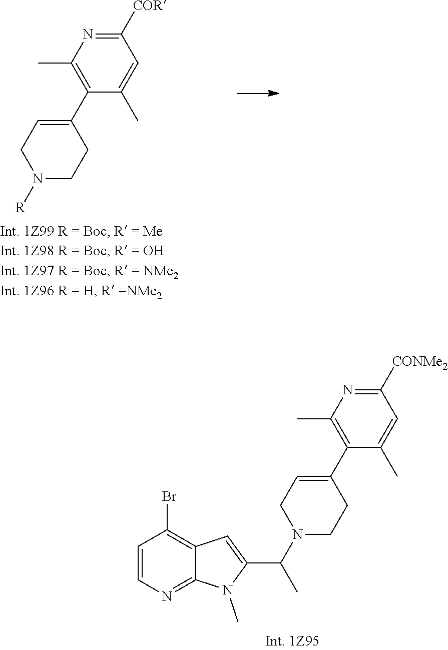

PdDPPFCl₂-DCM (0.2 g), methyl 5-bromo-4,6-dimethylpicolinate (1.1 g), K₂CO₃ (2 g), and tert-butyl 4-(4,4,5,5-tetramethyl-1,3,2-dioxaborolan-2-yl)-3,6-dihydropyridine-1 (2H)-carboxylate (1.5 g) were stirred 12 h in dioxane (8:1, 45 mL) at 80° C. under an inert atmosphere. The mixture was filtered and concentrated to give Int. 1Z99 1′-(tert-butoxycarbonyl)-2,4-dimethyl-1′,2′,3′,6′-tetrahydro-[3,4′-bipyridine]-6-carboxylic acid (1.4 g). LiOH·H₂O (0.3 g) and Int. 1Z99 (1.4 g) were stirred 12 h in MeOH/H₂O (1:1, 60 mL). The mixture was concentrated to give Int. 1Z98 1′-(tert-butoxycarbonyl)-2,4-dimethyl-1′,2′,3′,6′-tetrahydro-[3,4′-bipyridine]-6-carboxylic acid (1.3 g, Li salt). Int. 1Z98 (1.4 g), dimethylamine hydrochloride (0.4 g) and HATU (1.7 g) were stirred 12 h in DMF (50 mL) and Et₃N (2.8 mL). The mixture was concentrated. The OL (water/EtOAc) was concentrated to give Int. 1Z97 tert-butyl 6-(dimethylcarbamoyl)-2,4-dimethyl-3′,6′-dihydro-[3,4′-bipyridine]-1′(2′H)-carboxylate (0.95 g). Int. 1Z97 (0.95 g) was stirred ON in MeOH/2.8M HCl in dioxane (1:1, 40 mL). The mixture was concentrated to give Int. 1Z96 N,N,2,4-tetramethyl-1′,2′,3′,6′-tetrahydro-[3,4′-bipyridine]-6-carboxamide (0.7 g, HCl salt). Int. 1AF89 (0.3 g) was stirred 0.5 h in toluene (20 mL) and SOCl₂ (0.61 mL) at 80° C. The mixture was concentrated. The residue, Int. 1Z96 (0.5 g), and Cs₂CO₃ (0.3 g) were stirred 12 h in DMF (10 mL). The OL (water/EtOAc) was concentrated and HPLC-purified to give Int. 1Z95 1′-(1-(4-bromo-1-methyl-1H-pyrrolo[2,3-b]pyridin-2-yl)ethyl)-N,N,2,4-tetramethyl-1′,2′,3′,6′-tetrahydro-[3,4′-bipyridine]-6-carboxamide (0.5 g).

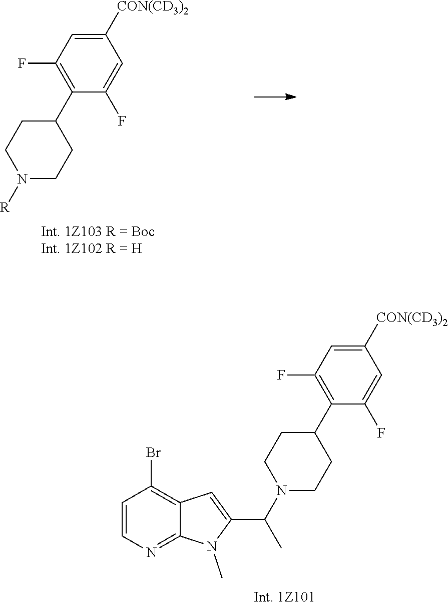

Int. 1Z87 (2 g), bis(methyl-d₃)amine hydrochloride (0.62 g), and HATU (3.3 g) were stirred 16 h in DMF (30 mL) and DIPEA (5.1 mL) at 0° C. to RT. The OL (water/EtOAc) was dried, concentrated, and purified by FC (PE/EtOAc 2:1) to give Int. 1Z103 tert-butyl 4-(4-(bis(methyl-d₃)carbamoyl)-2,6-difluorophenyl)-piperidine-1-carboxylate (1.4 g). Int. 1Z103 (1.4 g) was stirred 16 h in 2.2M HCl in dioxane (18 mL) at 0° C. to RT. The mixture was concentrated to give Int. 1Z102 3,5-difluoro-N,N-bis(methyl-d₃)-4-(piperidin-4-yl)benzamide (1.1 g, HCl salt). Ints. 1J1/1Z102 (0.50 g/0.60 g), KI (0.16 g), and K₂CO₃ (1.3 g) were stirred 16 h in ACN (20 mL) at 0° C. to RT. The OL (water/EtOAc) was concentrated and purified by FC (hexane/EtOAc 1:4) to give Int. 1Z101 4-(1-((4-bromo-1-methyl-1H-pyrrolo[2,3-b]pyridin-2-yl)methyl)piperidin-4-yl)-3,5-difluoro-N,N-bis(methyl-d₃)benzamide (0.45 g).

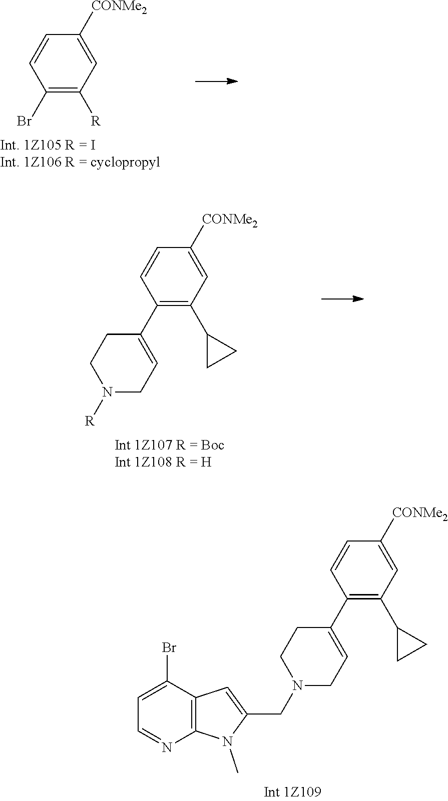

NaIO₄ (2.6 g) was added portion-wise to a mixture of I₂ (8.9 g) in sulfuric acid (100 mL). The mixture was stirred 0.5 h before 4-bromo-N,N-dimethylbenzamide (17 g) was added and stirring continued for 18 h. The mixture was diluted with water to precipitate a solid that was dried and crystallized from CCl₄ to give Int. 1Z105 4-bromo-3-iodo-N,N-dimethylbenzamide (17 g). PdDPPFCl₂-DCM (2.0 g), Int. 1Z105 (17 g), cyclopropylboronic acid (5.0 g), and K₂CO₃ (20 g) were refluxed ON in dioxane/H₂O (3:1, 200 mL) under an inert atmosphere. The mixture was concentrated and purified by FC (CHCl₃ to CHCl₃/ACN 1:1) to give Int. 1Z106 4-bromo-3-cyclopropyl-N,N-dimethylbenzamide (12 g). PdDPPFCl₂-DCM (1.9 g), Int. 1Z106 (12 g), tert-butyl 4-(4,4,5,5-tetramethyl-1,3,2-dioxaborolan-2-yl)-3,6-dihydropyridine-1 (2H)-carboxylate (21 g), and K₂CO₃ (19 g) were refluxed ON in dioxane/H₂O (6:1, 140 mL) under an inert atmosphere. The mixture was concentrated and purified by FC (CHCl₃ to CHCl₃/ACN 1:1) to give Int. 1Z107 tert-butyl 4-(2-cyclopropyl-4-(dimethylcarbamoyl)phenyl)-3,6-dihydropyridine-1 (2H)-carboxylate (4.1 g). Int. 1Z107 (4.1 g) was stirred in ON 4M HCl in dioxane (50 mL). The mixture was concentrated and triturated in EtOAc to give Int. 1Z108 3-cyclopropyl-N,N-dimethyl-4-(1,2,3,6-tetrahydropyridin-4-yl)benzamide (0.10 g, HCl salt). STAB (0.30 g) and Ints. 1AB8/1Z108 (0.1 g/0.1 g) were stirred ON in DCE/Et₃N (73:1, 10.1 mL) at 0° C. to RT. The mixture was concentrated, dissolved in THF, filtered through silica, and concentrated to give Int. 1Z109 4-(1-((4-bromo-1-methyl-1H-pyrrolo[2,3-b]pyridin-2-yl)methyl)-1,2,3,6-tetrahydropyridin-4-yl)-3-cyclopropyl-N,N-dimethylbenzamide (0.19 g).

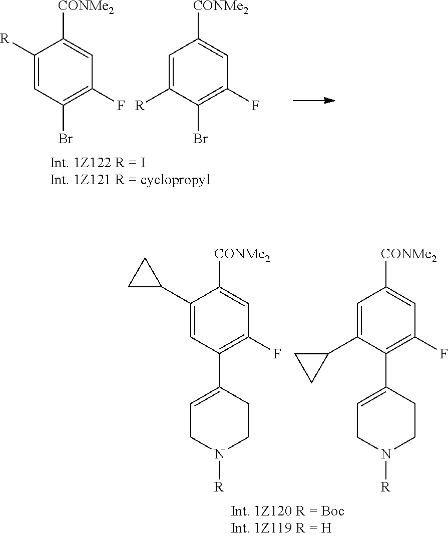

NaIO₄ (2.3 g) was added to a mixture of I₂ (8.0 g) in sulfuric acid (100 mL) portion-wise and stirred 0.5 h. 4-Bromo-3-fluoro-N,N-dimethylbenzamide (17 g) was added and stirring continued 18 h. The reaction mixture was diluted with water to precipitate a solid that was dried and precipitated from CCl₄ to give Int. 1Z122, a mixture of 4-bromo-5-fluoro-2-iodo-N,N-dimethylbenzamide and 4-bromo-3-fluoro-5-iodo-N,N-dimethylbenzamide mixture (13 g). PdDPPFCl₂-DCM (1.4 g), Int. 1Z122 (13 g), cyclopropylboronic acid (3.6 g), and K₂CO₃ (14 g) were refluxed ON in dioxane/H₂O (3:1, 200 mL) under an inert atmosphere. The mixture was concentrated and purified by FC (CHCl₃ to CHCl₃/ACN 1:1) to give Int. 1Z121, a mixture of 4-bromo-2-cyclopropyl-5-fluoro-N,N-dimethylbenzamide and 4-bromo-3-cyclopropyl-5-fluoro-N,N-dimethylbenzamide mixture (7 g). PdDPPFCl₂-DCM (1.0 g), Int. 1Z121 (7 g), tert-butyl 4-(4,4,5,5-tetramethyl-1,3,2-dioxaborolan-2-yl)-3,6-dihydropyridine-1 (2H)-carboxylate (11 g), and K₂CO₃ (10 g) were refluxed ON in dioxane/H₂O (6:1, 140 mL) under an inert atmosphere. The mixture was concentrated and purified by FC (CHCl₃ to CHCl₃/ACN 1:1) to give Int. 1Z120, a mixture of tert-butyl 4-(5-cyclopropyl-4-(dimethylcarbamoyl)-2-fluorophenyl)-3,6-dihydropyridine-1 (2H)-carboxylate and tert-butyl 4-(2-cyclopropyl-4-(dimethylcarbamoyl)-6-fluoro-phenyl)-3,6-dihydropyridine-1 (2H)-carboxylate mixture (2.6 g). Int. 1Z120 (2.6 g) was stirred in ON 4M HCl in dioxane (50 mL). The mixture was concentrated and triturated in EtOAc to give Int. 1Z119 2-cyclopropyl-5-fluoro-N,N-dimethyl-4-(1,2,3,6-tetrahydropyridin-4-yl)benzamide and 3-cyclopropyl-5-fluoro-N,N-dimethyl-4-(1,2,3,6-tetrahydropyridin-4-yl)benzamide mixture (2.0 g, HCl salt).

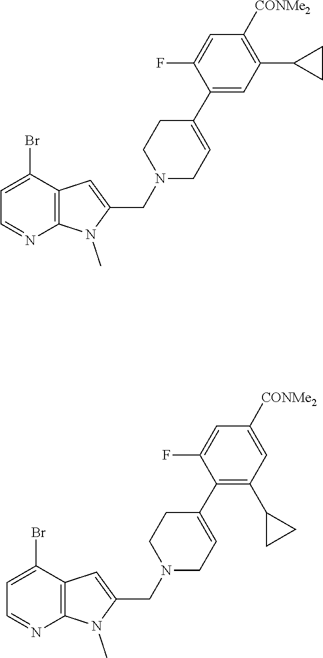

STAB (0.30 g) and Ints. 1AB8/1Z119 (0.1 g/0.1 g) were stirred ON in DCE/Et₃N (73:1, 10.1 mL) at 0° C. to RT. The residue after concentration dissolved in THF, filtered through silica, and concentrated to give a mixture of Ints. 1Z123/1Z124 4-(1-((4-bromo-1-methyl-1H-pyrrolo[2,3-b]pyridin-2-yl)methyl)-1,2,3,6-tetrahydropyridin-4-yl)-2-cyclopropyl-5-fluoro-N,N-dimethylbenzamide and 4-(1-((4-bromo-1-methyl-1H-pyrrolo[2,3-b]pyridin-2-yl)methyl)-1,2,3,6-tetrahydropyridin-4-yl)-3-cyclopropyl-5-fluoro-N,N-dimethylbenzamide (0.17 g).

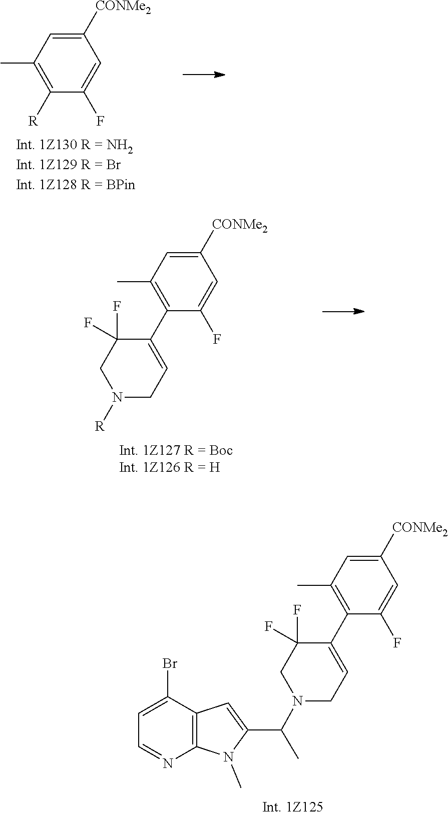

N-Iodosuccinimide (196 g) and 2-fluoro-6-methylaniline (100 g) were stirred 2 h in ACN (2.0 L) at 0° C. to RT. The OL (aq. Na₂S₂O₃/EtOAc) was dried, and concentrated to give Int. 1Z131 2-fluoro-4-iodo-6-methylaniline (180 g). Int. 1Z131 (30 g), dimethylamine hydrochloride (24 g), and PdDPPFCl₂-DCM (4.9 g) were stirred 16 h in dioxane/Et₃N (14:3, 364 mL) at 85° C. under 200 psi of CO. The mixture was filtered and concentrated. The OL (1M HCl/EtOAc) was neutralized to pH −8 with Na₂CO₃. The OL (water/EtOAc) was dried, and concentrated to give Int. 1Z130 4-amino-3-fluoro-N,N,5-trimethylbenzamide (17 g). CuBr₂ (85 g) and 1Z130 (15 g) were stirred 16 h in ACN (300 mL) and ^{t}BuONO (14 mL) at 0-85° C. The OL (aq. NH₃/PE) was dried, filtered and concentrated to give Int. 1Z129 4-bromo-3-fluoro-N,N,5-trimethylbenzamide (14 g). Int. 1Z129 (1 g), PdDPPFCl₂-DCM (0.31 g), bis(pinacolato)diboron (2.0 g), and KOAc (1.1 g) were stirred 1 h in dioxane (10 mL) at 110° C. under MW conditions. The mixture was filtered through celite, concentrated, and washed with pentane to give Int. 1Z128 3-fluoro-N,N,5-trimethyl-4-(4,4,5,5-tetramethyl-1,3,2-dioxaborolan-2-yl)benzamide (0.80 g). PdDPPFCl₂-DCM (0.11 g), Ints. 3E284/1Z128 (0.50 g/0.77 g), and Na₂CO₃ (0.43 g) were stirred 1.5 h in dioxane/H₂O (4:1, 10 mL) at 110° C. under MW conditions. The mixture was filtered. The OL (water/EtOAc) was concentrated and purified by FC (hexane/EtOAc 3:7) to give Int. 1Z127 tert-butyl 4-(4-(dimethylcarbamoyl)-2-fluoro-6-methylphenyl)-3,3-difluoro-3,6-dihydro-pyridine-1 (2H)-carboxylate (65 mg). Int. 1Z127 (0.50 g) was stirred 16 h in DCM/4M HCl in dioxane (2:1, 6 mL) at 0° C. to RT. The mixture was concentrated to give Int. 1Z126 4-(3,3-difluoro-1,2,3,6-tetrahydro-pyridin-4-yl)-3-fluoro-N,N,5-trimethylbenzamide (0.40 g, HCl salt). Ints. 1AF88/1Z126 (0.40 g/0.59 g), KI (0.17 g), and Cs₂CO₃ (3.3 g) were stirred 12 h in ACN (10 mL) at 0° C. to RT. The mixture was filtered, concentrated, and purified by FC (hexane/EtOAc 4:1) to give Int. 1Z125 4-(1-(1-(4-bromo-1-methyl-1H-pyrrolo[2,3-b]-pyridin-2-yl)ethyl)-3,3-difluoro-1,2,3,6-tetrahydropyridin-4-yl)-3-fluoro-N,N,5-trimethylbenzamide (0.13 g).

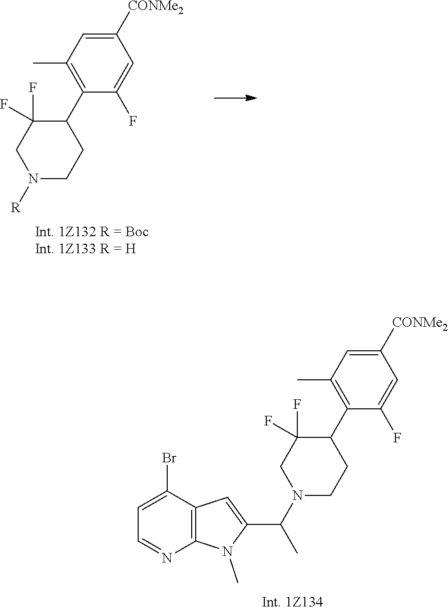

Int. 1Z127 (0.40 g) and PtO₂ (0.80 g) were hydrogenated 75 h at 70 psi in THF/EtOH/AcOH (8:8:1, 17 mL). The mixture was filtered and concentrated to give Int. 1Z132 tert-butyl 4-(4-(dimethyl-carbamoyl)-2-fluoro-6-methylphenyl)-3,3-difluoropiperidine-1-carboxylate (0.40 g). Int. 1Z133 4-(3,3-difluoropiperidin-4-yl)-3-fluoro-N,N,5-trimethylbenzamide (0.20 g, HCl salt) was prepared similarly to Int. 1Z126 from Int. 1Z132 (0.25 g). Ints. 1AF88/1Z133 (0.15 g/0.18 g), KI (64 mg), and Cs₂CO₃ (0.12 g) were stirred 16 h in ACN (10 mL) at 0° C. to RT. The OL (water/EtOAc) was dried, concentrated, and purified by FC (PE/EtOAc 1:1 to 1:2) to give Int. 1Z134 4-(1-(1-(4-bromo-1-methyl-1H-pyrrolo[2,3-b]pyridin-2-yl)ethyl)-3,3-difluoropiperidin-4-yl)-3-fluoro-N,N,5-trimethyl-benzamide (0.13 g).

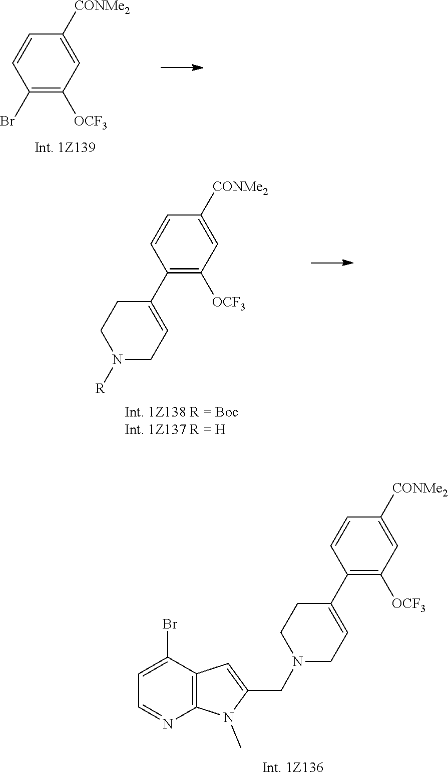

Di(1H-imidazol-1-yl)methanone (1.0 g) and 4-bromo-3-(trifluoromethoxy)benzoic acid (1.8 g) were refluxed 0.3 h in dioxane (50 mL). Dimethylamine hydrochloride (0.51 g) and Et₃N (5.2 mL) was added and stirring continued 10 h. The mixture was concentrated. The OL (aq. NaHSO₄/CHCl₃) was concentrated to give Int. 1Z139 4-bromo-N,N-dimethyl-3-(trifluoro(oxo)-λ6-methyl)benzamide (1.5 g). Int. 1Z139 (1.5 g), tert-butyl 4-(4,4,5,5-tetramethyl-1,3,2-dioxaborolan-2-yl)-3,6-dihydropyridine-1 (2H)-carboxylate (1.6 g), K₂CO₃ (2.0 g), and PdDPPFCl₂-DCM (0.20 g) were stirred ON in dioxane/H₂O (4:1, 20 mL) at 100° C. under an inert atmosphere. The mixture was concentrated and purified by FC to give Int. 1Z138 tert-butyl 4-(4-(dimethylcarbamoyl)-2-(trifluoromethoxy)phenyl)-3,6-dihydro-pyridine-1 (2H)-carboxylate (0.78 g). Int. 1Z138 (0.78 g) was stirred ON in 2.8M HCl in dioxane (10 mL). The mixture was concentrated to give Int. 1Z137 N,N-dimethyl-4-(1,2,3,6-tetrahydropyridin-4-yl)-3-(trifluoromethoxy)benzamide (0.43 g, HCl salt). Ints. 1J1/1Z137 (0.36 g/0.43 g) and K₂CO₃ (0.57 g) were stirred 11 h in DMF (10 mL). The mixture was diluted with water to precipitate Int. 1Z136 4-(1-((4-bromo-1-methyl-1H-pyrrolo[2,3-b]pyridin-2-yl)methyl)-1,2,3,6-tetrahydropyridin-4-yl)-N,N-dimethyl-3-(trifluoromethoxy)benzamide (0.14 g).

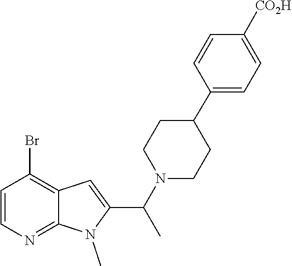 [SMILES: CC(c1cc2c(Br)ccnc2n1C)N1CCC(c2ccc(C(=O)O)cc2)CC1]

Int. C3. 4-(1-(1-(4-bromo-1-methyl-1H-pyrrolo[2,3-b]pyridin-2-yl)ethyl)piperidin-4-yl)benzoic acid (0.11 g) was prepared similarly to Int. 2C9 from Int. 3E37 (0.12 g).

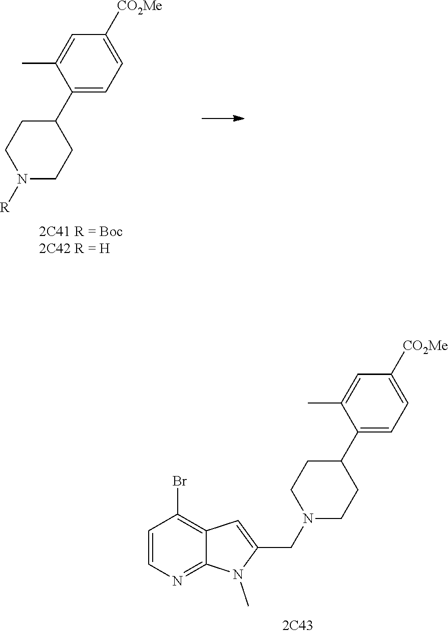

Methyl 4-bromo-3-methylbenzoate (2.5 g), tert-butyl 4-(4,4,5,5-tetramethyl-1,3,2-dioxaborolan-2-yl)-3,6-dihydropyridine-1 (2H)-carboxylate (4.0 g), NaHCO₃ (2.8 g), and PdDPPFCl₂-DCM (0.45 g) were degassed in dioxane/water (10:1, 30 mL) and stirred ON at 100° C. The mixture was diluted with EtOAc (100 mL), filtered, concentrated, and purified by FC (hexane/EtOAc 9:1 to 85:15) to give Int. 2C41 tert-butyl 4-(4-methoxy-carbonyl-2-methyl-phenyl)-3,6-dihydro-2H-pyridine-1-carboxylate (3.2 g). Int. 2C41 (0.25 g) was stirred ON in DCM/4M HCl in dioxane (4:1, 10 mL) and concentrated. The residue was triturated in pentane to give Int. 2C42 methyl 3-methyl-4-(1,2,3,6-tetra-hydropyridin-4-yl)benzoate (0.19 g, HCl salt). Ints. 1AB8/2C42 (1.2 g/1.2 g, HCl salt) were stirred 4 h in DCE/DIPEA (2:2:1, 7.3 mL) at 0° C. to RT. STAB (1.9 g) was added and stirring continued ON. The OL (EtOAc/water) was dried, concentrated, and purified by FC (hexane/EtOAc 7:3) to give Int. 2C43 methyl 4-(1-((4-bromo-1-methyl-1H-pyrrolo[2,3-b]pyridin-2-yl)methyl)-1,2,3,6-tetrahydropyridin-4-yl)-3-methyl-benzoate (1.0 g).

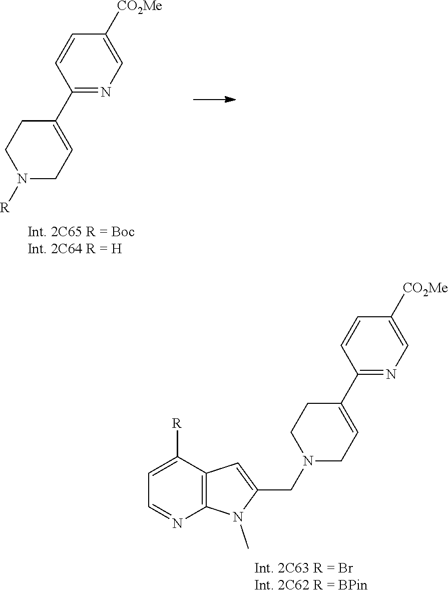

Methyl 6-bromonicotinate (5.0 g), tert-butyl 4-(4,4,5,5-tetramethyl-1,3,2-dioxaborolan-2-yl)-3,6-dihydropyridine-1 (2H)-carboxylate (7.8 g), K₂CO₃ (3.0 g) and PdDPPFCl₂-DCM (1.9 g) were stirred in dioxane/H₂O (4:1, 100 mL) ON at 100° C. under an inert atmosphere. The OL (water/EtOAc) was dried, concentrated and purified by FC (hexane/MTBE 1:0 to 0:1) to give Int. 2C65 1′-(tert-butyl) 5-methyl 3′,6′-dihydro-[2,4′-bipyridine]-1′,5 (2′H)-dicarboxylate (5.9 g). Int. 2C65 (2.0 g) was stirred 16 h in 2M HCl in dioxane (40 mL), concentrated, and washed with Et₂O to give Int. 2C64 methyl 1′,2′,3′,6′-tetrahydro-[2,4′-bipyridine]-5-carboxylate (1.8 g, HCl salt). Ints. 1AB8/2C64 (616 mg/750 mg) and 4 Å MS were stirred ON in DCE/Et₃N (9:1, 11.1 mL). (AcO)₃BHNa (1.6 g) was added and stirring continued for 3 h before it was concentrated and HPLC-purified to give Int. 2C63 methyl 1′-((4-bromo-1-methyl-1H-pyrrolo[2,3-b]pyridin-2-yl)methyl)-1′,2′,3′,6′-tetrahydro-[2,4′-bipyridine]-5-carboxylate (0.57 g). Int. 2C63 (0.29 g), bis(pinacolato)diboron (0.18 g), KOAc (0.19 g), and PdDPPFCl₂-DCM (53 mg) were stirred ON in dioxane (10 mL) at 80° C. under an inert atmosphere. The residue after concentration was dissolved in THF, filtered through silica, and concentrated to give Int. 2C62 methyl 1′-((1-methyl-4-(4,4,5,5-tetramethyl-1,3,2-dioxaborolan-2-yl)-1H-pyrrolo[2,3-b]pyridin-2-yl)-methyl)-1′,2′,3′,6′-tetrahydro-[2,4′-bipyridine]-5-carboxylate (0.30 g).

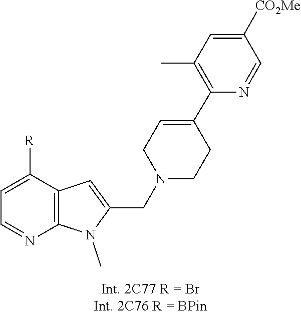

Ints. 1AB8/2C78 (0.50 g/1.0 g) and 4 Å MS were stirred ON in DCE/Et₃N (15:1, 15.9 mL). (AcO)₃BHNa (1.0 g) was added and stirring continued 3 h. The mixture was concentrated and HPLC-purified to give Int. 2C77 methyl 1′-((4-bromo-1-methyl-1H-pyrrolo[2,3-b]pyridin-2-yl)methyl)-3-methyl-1′,2′,3′,6′-tetrahydro-[2,4′-bipyridine]-5-carboxylate (0.61 g). Int. 2C77 (0.30 g), bis(pinacolato)diboron (0.30 mg), KOAc (0.30 mg) and PdDPPFCl₂-DCM (30 mg) were stirred ON in dioxane (3 mL) at 80° C. under an inert atmosphere. The residue after concentration was dissolved in THF, filtered through silica and concentrated to give Int. 2C76 methyl 3-methyl-1′-((1-methyl-4-(4,4,5,5-tetramethyl-1,3,2-dioxaborolan-2-yl)-1H-pyrrolo[2,3-b]pyridin-2-yl)methyl)-1′,2′,3′,6′-tetrahydro-[2,4′-bipyridine]-5-carboxylate (0.30 g).

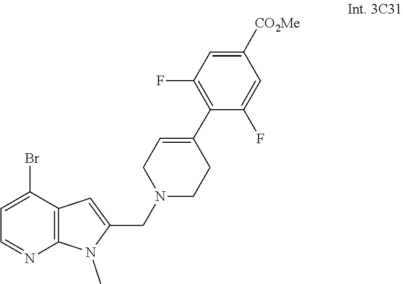 [SMILES: COC(=O)c1cc(F)c(C2=CCN(Cc3cc4c(Br)ccnc4n3C)CC2)c(F)c1]

Ints. 1AB8/3C25 (8.0 g/12.6 g) were stirred 16 h in DCE (80 mL) and DIPEA (35 mL) at 0° C. to RT under an inert atmosphere. STAB (28 g) was added in portions at 0° C. and stirring was continued for 12 h. The OL (water/DCM) was dried, filtered, concentrated, and purified by FC (PE/EtOAc 3:2) to give Int. 3C31 methyl 4-(1-((4-bromo-1-methyl-1H-pyrrolo[2,3-b]pyridin-2-yl)methyl)-1,2,3,6-tetrahydropyridin-4-yl)-3,5-difluorobenzoate (7.3 g).

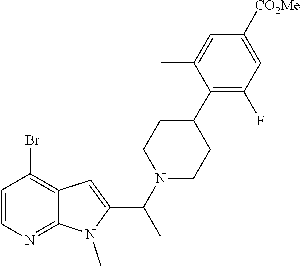 [SMILES: COC(=O)c1cc(C)c(C2CCN(C(C)c3cc4c(Br)ccnc4n3C)CC2)c(F)c1]

Ints. 1AF88/2G97 (2.0 g/2.3 g), KI (0.85 g) and Cs₂CO₃ (9.5 g) were mixed in ACN (20 mL) at 0° C. and stirred for 16 h at RT filtered and concentrated. The residue was purified by FC (hexane/EtOAc 4:1) to give Int. 2G85 methyl 4-(1-(1-(4-bromo-1-methyl-1H-pyrrolo[2,3-b]pyridin-2-yl)ethyl)piperidin-4-yl)-3-fluoro-5-methylbenzoate (2.0 g).

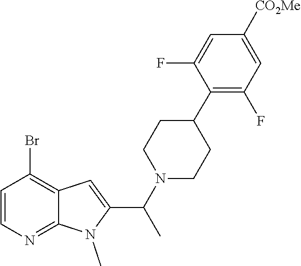 [SMILES: COC(=O)c1cc(F)c(C2CCN(C(C)c3cc4c(Br)ccnc4n3C)CC2)c(F)c1]

Int. 1AF89 (1.5 g) was stirred 0.3 h in toluene/SOCl₂ (7:3, 14.3 mL) at 90° C. The residue after concentration, Cs₂CO₃ (7.7 g), KI (0.49 g), and Int. 3C16 (2.1 g) were stirred 16 h in ACN (10 mL) at 0° C. to RT under an inert atmosphere. The reaction mixture was concentrated and triturated in water to precipitate a solid that was washed with water, dried, and purified by FC (PE/EtOAc 9:1 to 17:3) to give Int. 2G96 methyl 4-(1-(1-(4-bromo-1-methyl-1H-pyrrolo[2,3-b]pyridin-2-yl)ethyl)piperidin-4-yl)-3,5-difluorobenzoate (1.5 g).

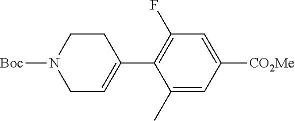 [SMILES: COC(=O)c1cc(C)c(C2=CCN(C(=O)OC(C)(C)C)CC2)c(F)c1]

Int. 1AF90 (15 g), Boc₂O (17 g) and DMAP (0.64 g) were mixed in DCM/Et₃N (4:1, 187 mL) at 0° C. and stirred 16 h and filtered. The OL (water/DCM) was dried, concentrated, and purified by FC (hexane/EtOAc 2:1) to give Int. 2G105 tert-butyl 4-(2-fluoro-4-(methoxycarbonyl)-6-methylphenyl)-3,6-dihydropyridine-1 (2H)-carboxylate (14 g).

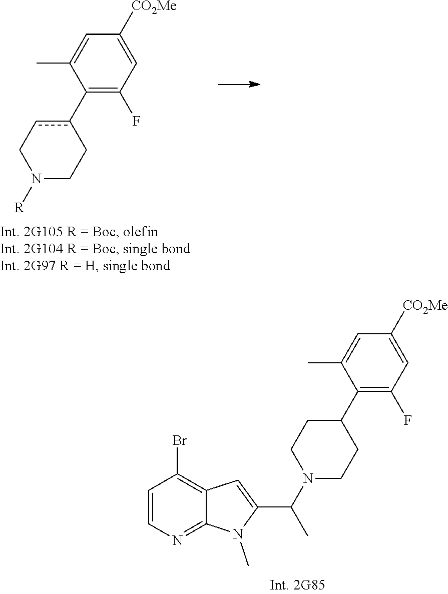

Int. 2G105 (14 g) and Pd/C (7 g) was hydrogenated 16 h in MeOH (140 mL), filtered, concentrated to give Int. 2G104 tert-butyl 4-(2-fluoro-4-(methoxycarbonyl)-6-methylphenyl)piperidine-1-carboxylate (13.2 g). Int. 2G104 (13.2 g) was stirred 16 h in DCM/4M HCl in dioxane (2:5, 105 mL) at 0° C. to RT. The mixture was concentrated and washed with pentane to give Int. 2G97 methyl 3-fluoro-5-methyl-4-(piperidin-4-yl)benzoate (9.0 g, HCl salt). Ints. 1AF88/2G97 (2.0 g/2.3 g), KI (0.85 g), and Cs₂CO₃ (9.5 g) were stirred 16 h in ACN (20 mL) at 0° C. to RT. The mixture was filtered and concentrated. The residue was purified by FC (hexane/EtOAc 4:1) to give Int. 2G85 methyl 4-(1-(1-(4-bromo-1-methyl-1H-pyrrolo[2,3-b]pyridin-2-yl)ethyl)piperidin-4-yl)-3-fluoro-5-methylbenzoate (2.0 g).

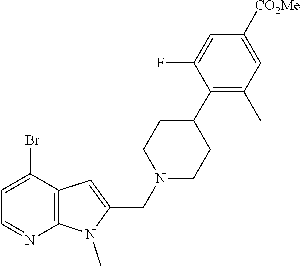 [SMILES: COC(=O)c1cc(C)c(C2CCN(Cc3cc4c(Br)ccnc4n3C)CC2)c(F)c1]

DIPEA (15 mL) was added to a solution of Ints. 1AB8/2G97 (8.0 g/12.5 g) in DCE/DMSO (5:1, 600 mL) at 0° C. and stirred for 16 h at RT. STAB (28 g) was added and stirring continued for 16 h. The OL (water/DCM/MeOH) was dried, filtered, concentrated and purified by FC (hexane/MeOH 4:1 to 2:1) to give Int. 2G115 methyl 4-(1-((4-bromo-1-methyl-1H-pyrrolo[2,3-b]pyridin-2-yl)methyl)piperidin-4-yl)-3-fluoro-5-methylbenzoate (8.0 g).

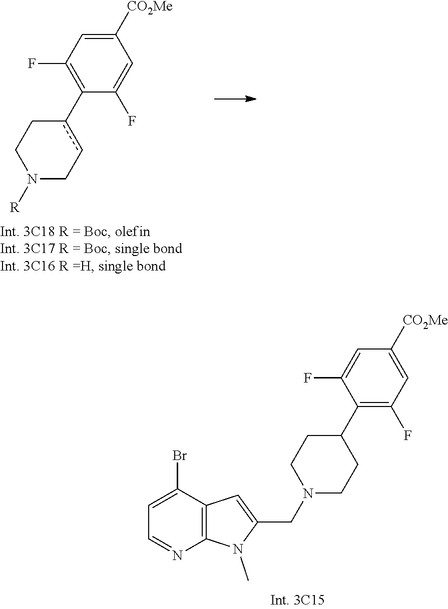

Methyl 4-bromo-3,5-difluorobenzoate (100 g), tert-butyl 4-(4,4,5,5-tetramethyl-1,3,2-dioxaborolan-2-yl)-3,6-dihydropyridine-1 (2H)-carboxylate (160 g), PdDPPFCl₂-DCM (16 g), and NaHCO₃ (100 g) were stirred 16 h in dioxane/H₂O (10:3, 1.3 L) at 100° C. under an inert atmosphere. The mixture was filtered. The OL (brine/EtOAc) was dried, concentrated, and purified by FC (PE/EtOAc 1:0 to 9:1) to give Int. 3C18 tert-butyl 4-(2,6-difluoro-4-(methoxycarbonyl)phenyl)-3,6-dihydropyridine-1 (2H)-carboxylate (45 g). Int. 3C18 (100 g) and 5% wet Pd/C (20 g) were stirred 16 h in MeOH (0.7 L) under an atmosphere of hydrogen. The mixture was filtered and concentrated to give Int. 3C17 tert-butyl 4-(2,6-difluoro-4-(methoxycarbonyl)phenyl)piperidine-1-carboxylate (90 g). Int. 3C17 (20 g) was stirred 16H in DCM/4M HCl in dioxane (2:1, 600 mL) at 0° C. to RT. The mixture was concentrated and washed with Et₂O to give Int. 3C16 methyl 3,5-difluoro-4-(piperidin-4-yl)benzoate (12 g, HCl salt). Ints. 1AB8/3C16 (3.5 g/3.6 g) were stirred 16 h in DCE/DMSO/DIPEA (7:3:1, 112 mL). STAB (12 g) was added at 0° C. and the mixture was stirred 16 h at 0° C. to RT. The OL (water/DCM) was dried, concentrated, and purified by FC (hexane/EtOAc 9:1) to give Int. 3C15 methyl 4-(1-((4-bromo-1-methyl-1H-pyrrolo[2,3-b]pyridin-2-yl)methyl)piperidin-4-yl)-3,5-difluorobenzoate (4.0 g).

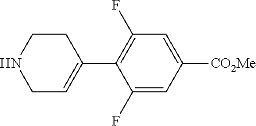 [SMILES: COC(=O)c1cc(F)c(C2=CCNCC2)c(F)c1]

Int. 3C25. methyl 3,5-difluoro-4-(1,2,3,6-tetrahydropyridin-4-yl)benzoate (16 g, HCl salt) was prepared similarly to Int. 3C16 from Int. 3C18 (20 g).

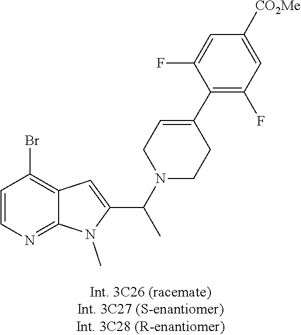 [SMILES: COC(=O)c1cc(F)c(C2=CCN(C(C)c3cc4c(Br)ccnc4n3C)CC2)c(F)c1]

Int. 1AF89 (9.0 g) was stirred 0.5 h in toluene/SOCl₂ (6:1, 174 mL) at 90° C. The residue after concentration, Cs₂CO₃ (43 g), KI (2.7 g), and Int. 3C25 (10 g) were stirred 16 h in ACN (250 mL) at 0° C. to RT under an inert atmosphere. The mixture was concentrated. The OL (water/EtOAc) was dried, concentrated, and purified by FC (PE/EtOAc 4:1) to give Int. 3C26 methyl 4-(1-(1-(4-bromo-1-methyl-1H-pyrrolo[2,3-b]pyridin-2-yl)ethyl)-1,2,3,6-tetrahydropyridin-4-yl)-3,5-difluorobenzoate (7.1 g). Int. 3C26 (0.85 g) was resolved by SFC on a SFC-150-008 instrument fitted with a Chiralpak-AD-H 150×25 mm 5 μm column operated at 30° C. and an eluent of 65% CO₂ and 35% MeOH (80 g/min) and a back pressure of 100 bar to give Int. 3C27 (0.30 g, first peak) and Int. 3C28 (0.30 g, second peak) methyl (S)-4-(1-(1-(4-bromo-1-methyl-1H-pyrrolo[2,3-b]pyridin-2-yl)ethyl)-1,2,3,6-tetrahydropyridin-4-yl)-3,5-difluorobenzoate and methyl (R)-4-(1-(1-(4-bromo-1-methyl-1H-pyrrolo[2,3-b]pyridin-2-yl)ethyl)-1,2,3,6-tetrahydropyridin-4-yl)-3,5-difluorobenzoate. The absolute configurations of these compounds were not determined.

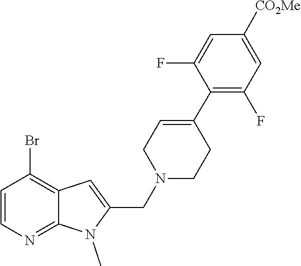 [SMILES: COC(=O)c1cc(F)c(C2=CCN(Cc3cc4c(Br)ccnc4n3C)CC2)c(F)c1]

Ints. 1AB8/3C25 (8.0 g/12.6 g) were stirred 16 h in DCE (80 mL) and DIPEA (35 mL) at 0° C. to RT under an inert atmosphere. STAB (28 g) was added in portions at 0° C. and stirring was continued for 12 h. The OL (water/DCM) was dried, concentrated, and purified by FC (PE/EtOAc 3:2) to give Int. 3C31 methyl 4-(1-((4-bromo-1-methyl-1H-pyrrolo[2,3-b]pyridin-2-yl)methyl)-1,2,3,6-tetrahydropyridin-4-yl)-3,5-difluorobenzoate (7.3 g).

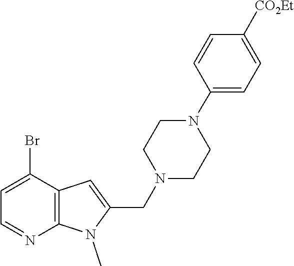 [SMILES: CCOC(=O)c1ccc(N2CCN(Cc3cc4c(Br)ccnc4n3C)CC2)cc1]

ZnCl₂ (1M in Et₂O, 31 mL) was added to a solution of Int. 1AB8 (2.7 g) and ethyl 4-(piperazin-1-yl)-benzoate (2.4 g) in THF (30 mL) at 0° C. and stirred 16 h at RT. NaCNBH₃ (3.4 g) was added and stirring continued for 16 h. The OL (water/EtOAc) was dried, concentrated, and purified by FC (PE/EtOAc 1:1) to give Int. 3C59 ethyl 4-(4-((4-bromo-1-methyl-1H-pyrrolo[2,3-b]pyridin-2-yl)methyl)-piperazin-1-yl)benzoate (2.2 g).

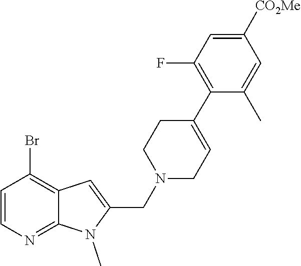 [SMILES: COC(=O)c1cc(C)c(C2=CCN(Cc3cc4c(Br)ccnc4n3C)CC2)c(F)c1]

DIPEA (7.3 mL) was added to a solution of Ints. 1AB8/1AF90 (2.0 g/2.9 g) at 0° C. and stirred in DCE (10 mL) for 16 h at RT under an inert atmosphere. STAB (5.2 g) was added at 0° C. and stirring continued for 2 h. The OL (water/EtOAc) was dried, concentrated, and purified by FC (hexane/EtOAc 3:2) to give Int. 3C73 methyl 4-(1-((4-bromo-1-methyl-1H-pyrrolo[2,3-b]pyridin-2-yl)methyl)-1,2,3,6-tetrahydropyridin-4-yl)-3-fluoro-5-methylbenzoate (1.3 g).

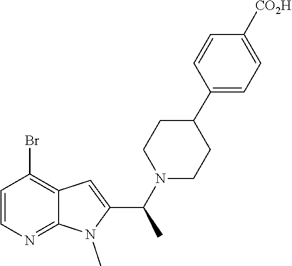 [SMILES: C[C@@H](c1cc2c(Br)ccnc2n1C)N1CCC(c2ccc(C(=O)O)cc2)CC1]

Int. 3E35 (1.7 g) and LiOH—H₂O (1.1 g) were stirred 3 h in THF/MeOH/H₂O (4:2:1, 25 mL) at 0° C. to RT and concentrated. The AL (water/Et₂O) was acidified with KHSO₄ to precipitate Int. 2C36 (S)-4-(1-(1-(4-bromo-1-methyl-1H-pyrrolo[2,3-b]pyridin-2-yl)ethyl)piperidin-4-yl)benzoic acid (1.5 g). The absolute configuration was determined for derivatives 1AF76 and 1AF76′.

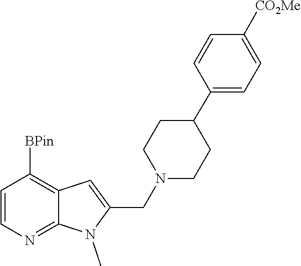

Int. 1AC5 (3.0 g), B₂Pin₂ (1.1 g), KOAc (2.0 g), and PdDPPFCl₂-DCM (50 mg) were degassed in dioxane (30 mL) and stirred ON at 90° C. The filtrate after filtration was concentrated and purified by FC (pentane/EtOAc 1:4) to give Int. 2H14 methyl 4-[1-[[1-methyl-4-(4,4,5,5-tetramethyl-1,3,2-dioxaborolan-2-yl)pyrrolo[2,3-b]pyridin-2-yl]methyl]-4-piperidyl]benzoate (1.7 g).

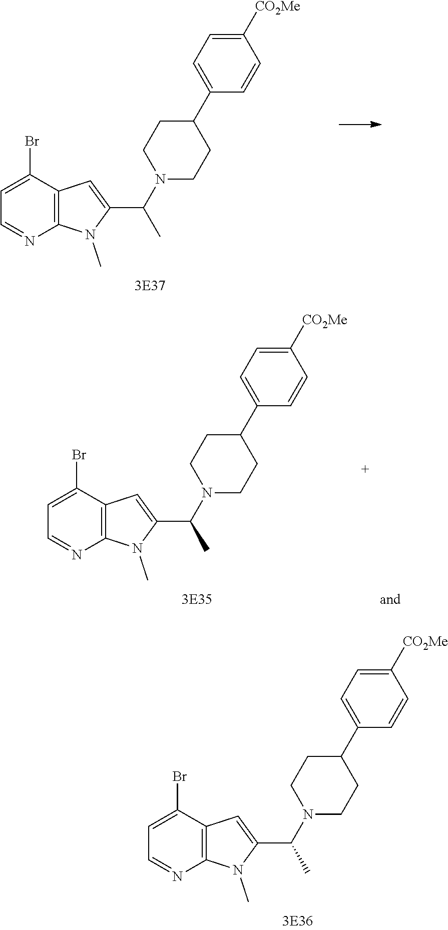 [SMILES: COC(=O)c1ccc(C2CCN(C(C)c3cc4c(Br)ccnc4n3C)CC2)cc1 ; COC(=O)c1ccc(C2CCN([C@@H](C)c3cc4c(Br)ccnc4n3C)CC2)cc1 ; COC(=O)c1ccc(C2CCN([C@H](C)c3cc4c(Br)ccnc4n3C)CC2)cc1]

Int. 1AF21 (1.0 g) was stirred 0.3 h in toluene/SOCl₂ (7.1:1, 22.8 mL) at 85-90° C. The residue after concentration, Cs₂CO₃ (3.8 g), KI (0.33 g), and methyl 4-(piperidin-4-yl)-benzoate (1.3 g, HCl salt) were stirred ON in ACN (25 mL) at 0° C. to RT. The mixture was concentrated to half volume and diluted with water to precipitate Int. 3E37 methyl 4-(1-(1-(4-bromo-1-methyl-1H-pyrrolo[2,3-b]pyridin-2-yl)ethyl)-piperidin-4-yl)benzoate (1.4 g). Int. 3E37 (7.4 g) was resolved by SFC using a Chiralpak IA 250×25 5 μm column operated at 30° C. using an eluent of 60% CO₂ and 40% MeOH at a flow rate of 90 g/min and a back pressure of 100 bar to give Int. 3E35 methyl (S)-4-(1-(1-(4-bromo-1-methyl-1H-pyrrolo[2,3-b]pyridin-2-yl)ethyl)piperidin-4-yl)benzoate (3.0 g, first eluting isomer) and Int. 3E36 methyl (R)-4-(1-(1-(4-bromo-1-methyl-1H-pyrrolo[2,3-b]pyridin-2-yl)ethyl)piperidin-4-yl)benzoate (3.0 g, second eluting isomer). The absolute configuration was determined for derivatives 1AF76 and 1AF76′.

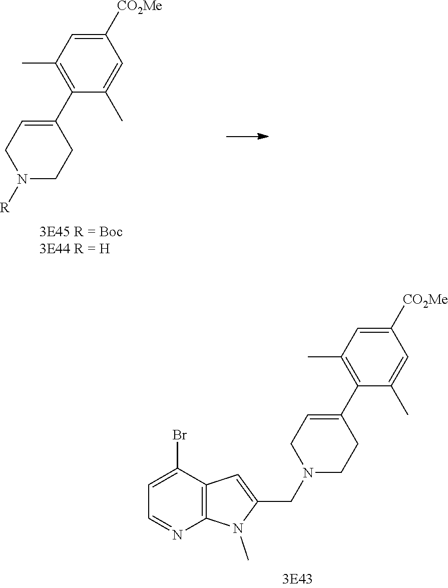

Methyl 4-bromo-3,5-dimethyl-benzoate (0.50 g), tert-butyl 4-(4,4,5,5-tetramethyl-1,3,2-dioxaborolan-2-yl)-3,6-dihydropyridine-1 (2H)-carboxylate (0.70 g), Na₂CO₃ (0.26 g), and PdDPPFCl₂-DCM (84 mg) were degassed in dioxane/water (9:1; 8 mL) and stirred ON at 90° C. The OL (water/EtOAc) was concentrated and purified by FC (hexane/EtOAc 7:3) to give Int. 3E45 tert-butyl 4-(4-methoxycarbonyl-2,6-dimethyl-phenyl)-3,6-dihydro-2H-pyridine-1-carboxylate (0.55 g). This material was stirred 3 h in DCM/4M HCl in dioxane (5:4; 9 mL) at 0° C. to RT and concentrated to give Int. 3E44 methyl 3,5-dimethyl-4-(1,2,3,6-tetrahydro-pyridin-4-yl)benzoate (0.44 g, HCl salt). Ints. 1AB8/3E44 (0.23 g/0.30 g) were stirred 4 h in DCE/DIPEA (12.5:1, 5.4 mL). STAB (0.41 g) was added and stirring continued ON. The OL (water/EtOAc) was dried, concentrated, and purified by FC (hexane/EtOAc 7:3) to give Int. 3E43 methyl 4-[1-[(4-bromo-1-methyl-pyrrolo[2,3-b]pyridin-2-yl)methyl]-3,6-dihydro-2H-pyridin-4-yl]-3,5-dimethyl-benzoate (0.33 g).

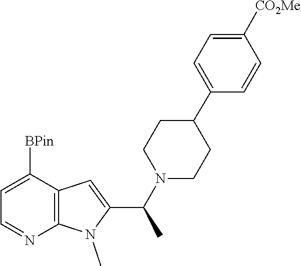

Int. 3E35 (2.0 g), B₂Pin₂ (1.7 g), PdDPPFCl₂-DCM (0.18 g), KOAc (0.9 g) were degassed in dioxane (15 mL) and stirred 5 h at 95-100° C., filtered, concentrated, and triturated in Et₂O to give Int. 3E170 methyl 4-[1-[(1S)-1-[1-methyl-4-(4,4,5,5-tetramethyl-1,3,2-dioxaborolan-2-yl)-pyrrolo[2,3-b]pyridin-2-yl]-ethyl]-4-piperidyl]benzoate (2.3 g).

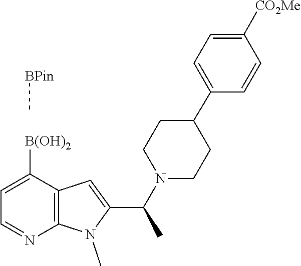

Int. 3E35 (0.50 g), B₂Pin₂ (0.41 g), PdDPPFCl₂-DCM (44 mg), KOAc (0.21 g) were degassed in dioxane (10 mL) and stirred 5 h at 95-100° C., filtered, concentrated, and triturated in Et₂O to give a mixture of Int. 3E170 and 3E177 (S)-(2-(1-(4-(4-(methoxycarbonyl)phenyl)piperidin-1-yl)-ethyl)-1-methyl-1H-pyrrolo[2,3-b]pyridin-4-yl)boronic acid and methyl (S)-4-(1-(1-(1-methyl-4-(4,4,5,5-tetramethyl-1,3,2-dioxaborolan-2-yl)-1H-pyrrolo[2,3-b]pyridin-2-yl)ethyl)piperidin-4-yl)benzoate (0.50 g).

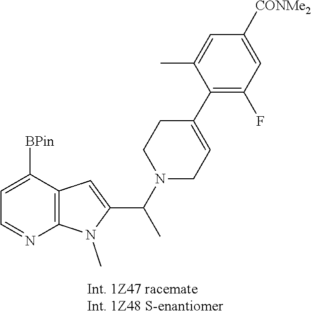

PdDPPFCl₂-DCM (40 mg), Int. 1Z69 (0.24 g), bis(pinacolato)diboron (0.15 g), and KOAc (0.14 g) were stirred ON in dioxane (5 mL) at 100° C. under an inert atmosphere. The mixture was concentrated and purified by FC (THF) to give Int. 1Z47 3-fluoro-N,N,5-trimethyl-4-(1-(1-(1-methyl-4-(4,4,5,5-tetramethyl-1,3,2-dioxaborolan-2-yl)-1H-pyrrolo[2,3-b]pyridin-2-yl)ethyl)-1,2,3,6-tetrahydropyridin-4-yl)benzamide (0.27 g). Int. 1AF84′ (0.20 g), bis(pinacolato)diboron (0.20 g), PdDPPFCl₂-DCM (32 mg), and KOAc (0.12 g) were stirred 6 h in dioxane (10 mL) at 90° C. under an inert atmosphere. The mixture was filtered through celite, concentrated, dissolved in Et₂O, filtered, concentrated, washed with pentane, and dried to give Int. 1Z48 (S)-3-fluoro-5-methyl-N,N-bis(methyl-d₃)-4-(1-(1-(1-methyl-4-(4,4,5,5-tetramethyl-1,3,2-dioxaborolan-2-yl)-1H-pyrrolo[2,3-b]pyridin-2-yl)ethyl)-1,2,3,6-tetrahydropyridin-4-yl)benzamide (0.14 g).

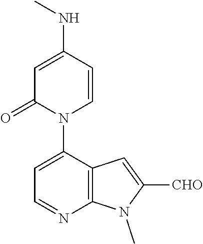 [SMILES: CNc1ccn(-c2ccnc3c2cc(C=O)n3C)c(=O)c1]

Int. 1AB8 (1.0 g), 4-(methylamino)pyridin-2 (1H)-one (0.57 g), K₃PO₄ (1.33 g), CuI (0.40 g), DMDCH (0.60 g) were degassed in dioxane (30 mL) and stirred at 110° C. ON and filtered. The OL (dioxane/water) was washed brine, dried, concentrated, and purified by FC (DCM/MeOH 9:1) to give Int. 1M1 1-Methyl-4-(4-(methylamino)-2-oxopyridin-1 (2H)-yl)-1H-pyrrolo[2,3-b]pyridine-2-carbaldehyde (0.34 g).

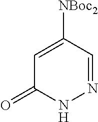

Int. 1AB9. tert-Butyl (tert-butoxycarbonyl)(6-oxo-1,6-dihydropyridazin-4-yl)carbamate

5-Aminopyridazin-3 (2H)-one (0.3 g), DMAP (0.17 g), and Boc₂O (2.36 g) were stirred ON in ACN/Et₃N (6:1, 11.7 mL) at 0° C. to RT ON and purified by FC (EtOAc/pentane 4:1) to give Int. 1AB9 (80 mg).

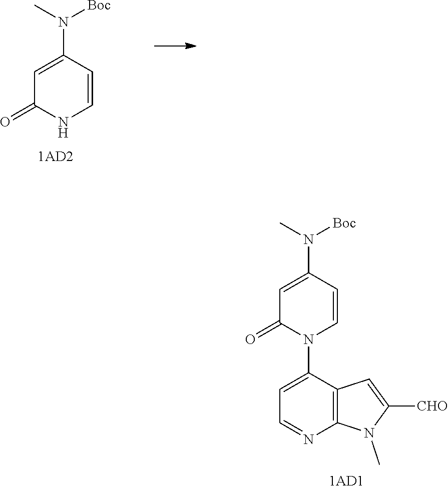 [SMILES: CN(C(=O)OC(C)(C)C)c1cc[nH]c(=O)c1 ; CN(C(=O)OC(C)(C)C)c1ccn(-c2ccnc3c2cc(C=O)n3C)c(=O)c1]

4-(Methylamino)pyridin-2 (1H)-one (0.9 g) was dissolved in 0.3M LiHMDS in THF (28 mL) at 0° C. Boc₂O (2.0 g) was added and stirring continued 2 h at 0° C. to 45-50° C. and ON at RT. The OL (aq. NH₄Cl/10% MeOH in DCM) was concentrated and purified by FC (DCM/MeOH 95:5) to give tert-butyl methyl(2-oxo-1,2-dihydropyridin-4-yl)carbamate (0.3 g). A larger portion of this material prepared similarly (2.1 g), Int. 1AB8 (2.0 g), CuI (0.8 g), DMDCH (1.2 g), and K₃PO₄ (2.7 g) were degassed in dioxane (50 mL) and stirred at 100° C. ON. This mixture was mixed with another batch prepared similarly on 1.0 g scale and filtered. The OL (EtOAc/water) was washed with brine, dried, concentrated, and purified by FC (EtOAc/pentane 3:2) to give Int. 1ADI tert-Butyl (1-(2-formyl-1-methyl-1H-pyrrolo[2,3-b]pyridin-4-yl)-2-oxo-1,2-dihydropyridin-4-yl)-(methyl)carbamate (2.5 g).

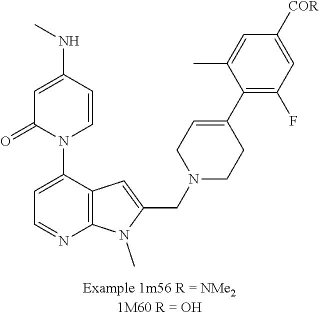

Int. 1M60. 3-fluoro-5-methyl-4-(1-((1-methyl-4-(4-(methylamino)-2-oxopyridin-1 (2H)-yl)-1H-pyrrolo[2,3-b]pyridin-2-yl)methyl)-1,2,3,6-tetrahydropyridin-4-yl)benzoic acid

Example 1m56 (2.4 g) and LiOH—H₂O (1.1 g) were stirred 3 h in THF/water (2.5:1, 28 mL) at 0° C. to 60° C. The residue after concentration was triturated in aq. HCl to give Int. 1M60 (2.4 g).

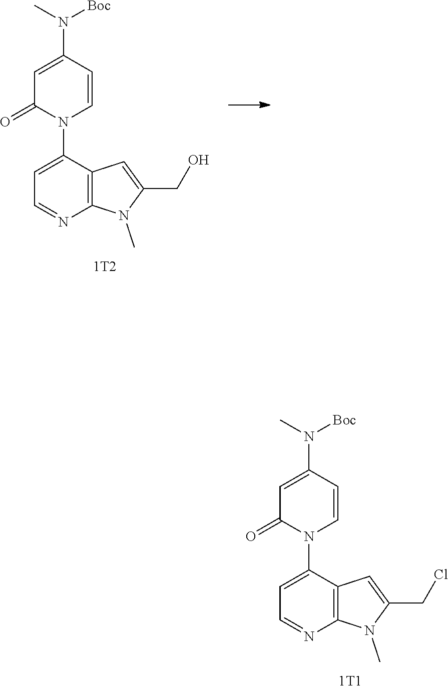 [SMILES: CN(C(=O)OC(C)(C)C)c1ccn(-c2ccnc3c2cc(CO)n3C)c(=O)c1 ; CN(C(=O)OC(C)(C)C)c1ccn(-c2ccnc3c2cc(CCl)n3C)c(=O)c1]

Int. 1ADI (0.10 g) and NaBH₄ (15 mg) were stirred 2 h in MeOH (10 mL) at 0° C. to RT and concentrated. The OL (water/EtOAc) was dried, and concentrated to give Int. 1T2 tert-butyl (1-(2-(hydroxymethyl)-1-methyl-1H-pyrrolo[2,3-b]pyridin-4-yl)-2-oxo-1,2-dihydropyridin-4-yl)(methyl)carbamate (60 mg). Int. 1T2 (0.10 g) was stirred ON in DCM/Et₃N (45:1, 10.2 mL) and MsCl (60 μL) at 0° C. to RT. The OL (water/DCM) was washed with sat. aq. NaHCO₃, dried, and concentrated to give Int. 1T1 tert-Butyl N-[1-[2-(chloromethyl)-1-methyl-pyrrolo[2,3-b]pyridin-4-yl]-2-oxo-4-pyridyl]-N-methyl-carbamate (50 mg).

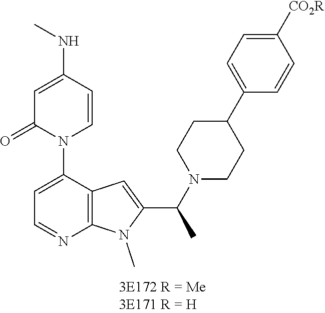

Ints. 3E35/1AD2 (0.40 g/0.29 g), CuI (0.33 g), K₂CO₃ (0.36 g), and DMDCH (0.25 g) were degassed in dioxane (10 mL) and stirred ON at 95-100° C. The OL (water/EtOAc) was dried, concentrated, and purified by FC (DCM/MeOH 95:5) to give Int. 3E172 methyl 4-[1-[(1S)-1-[4-[4-[tert-butoxycarbonyl(methyl)amino]-2-oxo-1-pyridyl]-1-methyl-pyrrolo[2,3-b]pyridin-2-yl]ethyl]-4-piperidyl]benzoate (0.33 g). A solution of this material (0.22 g) in dioxane/6M aq. HCl (1:1; 8 mL) was stirred ON at 70° C. and concentrated to give Int. 3E171 4-[1-[(1S)-1-[1-methyl-4-[4-(methylamino)-2-oxo-1-pyridyl]pyrrolo[2,3-b]pyridin-2-yl]ethyl]-4-piperidyl]benzoic acid (0.18 g, HCl salt).

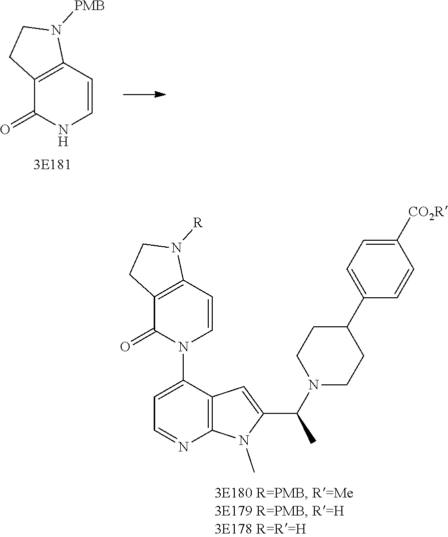

3,5-Dihydro-2H-furo[3,2-c]pyridin-4-one (2.15 g) was stirred 8 h in PMB-NH₂ (20 mL) at 180° C. and diluted with Et₂O to precipitate Int. 3E181 1-[(4-methoxyphenyl)-methyl]-3,5-dihydro-2H-pyrrolo[3,2-c]pyridin-4-one (2.35 g). Ints. 3E53/3E181 (0.30 g/0.2 g), CuI (0.25 g), K₂CO₃ (0.27 g), and DMDCH (0.19 g) were degassed in dioxane (10 mL) and stirred 2 h at 120° C. The OL (water/EtOAc) was dried, concentrated, and triturated in Et₂O to give Int. 3E180 methyl 4-[1-[(1S)-1-[4-[1-[(4-methoxyphenyl)methyl]-4-oxo-2,3-dihydropyrrolo-[3,2-c]pyridin-5-yl]-1-methyl-pyrrolo[2,3-b]pyridin-2-yl]ethyl]-4-piperidyl]benzoate (0.40 g). 0.15 g of this material and LiOH—H₂O (98 mg) were stirred 5 h in MeOH/THF/water (2:2:1; 5 mL), concentrated, and triturated in dilute aq. HCl to give Int. 3E179 4-[1-[(1S)-1-[4-[1-[(4-methoxyphenyl)-methyl]-4-oxo-2,3-dihydropyrrolo[3,2-c]-pyridin-5-yl]-1-methyl-pyrrolo[2,3-b]pyridin-2-yl]ethyl]-4-piperidyl]-benzoic acid (0.13 g). A solution of Int. 3E179 (0.15 g) in TFA/TfOH (10:1:1.65 mL) was stirred 1 h at 80° C. and concentrated to give Int. 3E178 (S)-4-(1-(1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)piperidin-4-yl)benzoic acid (0.15 g, TfOH salt).

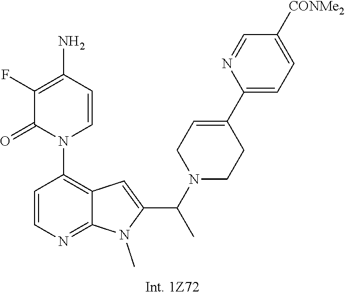

CuI (0.24 g), Int. 1Z71 (0.30 g), 4-amino-3-fluoropyridin-2 (1H)-one (82 mg), and K₃PO₄ (0.61 g) were stirred 2 h in dioxane (5 mL) and trans-N,N′-dimethylcyclohexane-1,2-diamine amine (0.20 mL) at 120° C. under an inert atmosphere. The OL (aq. NH₃/DCM/MeOH) was dried, filtered, concentrated, and purified by FC (DCM/MeOH 19:1) and HPLC-purified to give Int. 1Z72 1′-(1-(4-(4-amino-3-fluoro-2-oxopyridin-1 (2H)-yl)-1-methyl-1H-pyrrolo[2,3-b]pyridin-2-yl)ethyl)-N,N-dimethyl-1′,2′,3′,6′-tetrahydro-[2,4′-bipyridine]-5-carboxamide (75 mg).

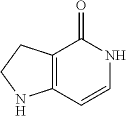

Int. 3E181 (2.3 g) was stirred 6 h in TFA (20 mL) at 0-75° C. The residue after concentration was dissolved in MeOH and concentrated. The residue was dissolved in MeOH and mixed with Amberlyst® A21 ion exchange resin, filtered, and dried to give Int. 1Z74 1,2,3,5-tetrahydro-4H-pyrrolo[3,2-c]pyridin-4-one (0.90 g).

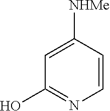

4-Chloropyridin-2-ol (10 g) was stirred ON in 2M methylamine in MeOH (100 mL) at 100° C. The mixture was concentrated and purified by FC (ACN/MeOH 1:0 to 3:1) to give Int. 1Z78 4-(methylamino)pyridin-2-ol (8 g).

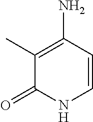

2-Methoxy-3-methylpyridin-4-amine (0.75 g), NaI (0.92 g), and (CH₃)₃SiCl (0.64 mL) were stirred 1 h in ACN (2 mL) at 85° C. under MW conditions. The mixture filtered and concentrated. The residue was dissolved in MeOH containing basic Amberlyst ion exchange resin. The mixture was filtered and dried to give Int. 1Z81 4-amino-3-methylpyridin-2 (1H)-one (0.30 g).

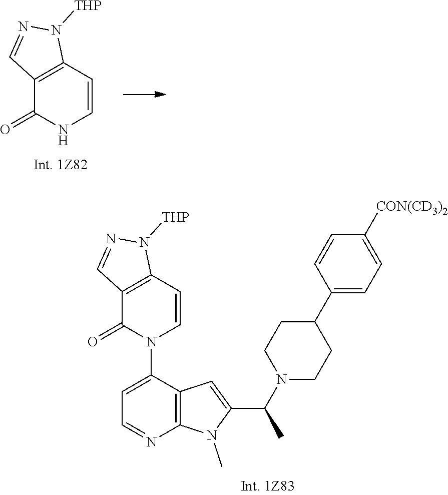

1,5-Dihydro-4H-pyrazolo[4,3-c]pyridin-4-one (2.0 g) and TsOH (1.3 g) were stirred 16 h in THF/3,4-dihydro-2H-pyran (22:3, 34 mL) at 0° C. to RT. The OL (aq. NaHCO₃/DCM) was dried, filtered, concentrated, and purified by FC (DCM/MeOH 19:1) to give Int. 1Z82 1-(tetrahydro-2H-pyran-2-yl)-1,5-dihydro-4H-pyrazolo[4,3-c]pyridin-4-one (1.3 g). CuI (73 mg), Ints. 1Z82/1AF77 (63 mg/0.10 g), and K₃PO₄ (0.18 g) were stirred 16 h in DMF (2 mL) and trans-N,N′-dimethylcyclohexane-1,2-diamine (60 μL) at 110° C. under an inert atmosphere. The mixture was filtered. The OL (aq. NH₃/DCM) was dried, filtered, concentrated, and purified by FC (DCM/MeOH 49:1) to give Int. 1Z83 N,N-bis(methyl-d₃)-4-(1-((1S)-1-(1-methyl-4-(4-oxo-1-(tetrahydro-2H-pyran-2-yl)-1,4-dihydro-5H-pyrazolo[4,3-c]pyridin-5-yl)-1H-pyrrolo[2,3-b]pyridin-2-yl)ethyl)piperidin-4-yl)benzamide (40 mg).

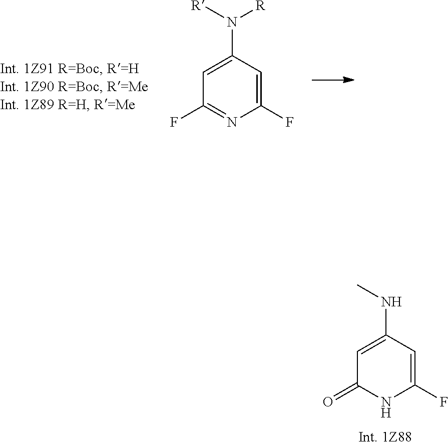

Di-tert-butyl dicarbonate (22 g) was added portion-wise to a mixture of 2,6-difluoropyridin-4-amine (12 g) and DMAP (1.1 g) in DCM (200 mL). The mixture was stirred 8 h and concentrated to give Int. 1Z91 tert-butyl (2,6-difluoropyridin-4-yl)carbamate (17 g). Int. 1Z91 (21 g) was added portion-wise to a solution of NaH (2.6 g) in DMF (100 mL) at 0° C. The mixture was stirred 1 h before MeI (6.2 mL) was added drop-wise and stirring continued 12 h. The OL (water/MTBE) was concentrated to give Int. 1Z90 tert-butyl (2,6-difluoropyridin-4-yl)(methyl)carbamate (22 g). Int. 1Z90 (17 g) was stirred 0.5 h in MeOH/2.8M HCl in dioxane (6:1, 350 mL). The mixture was concentrated to give Int. 1Z89 2,6-di-fluoro-N-methylpyridin-4-amine (10 g). Int. 1Z89 (10 g) was refluxed 15 h in 1.7M NaOH (0.2 L). The mixture was diluted with aq. citric acid. The OL (water/EtOAc) was concentrated to give Int. 1Z88 6-fluoro-4-(methylamino)pyridin-2 (1H)-one (1.2 g).

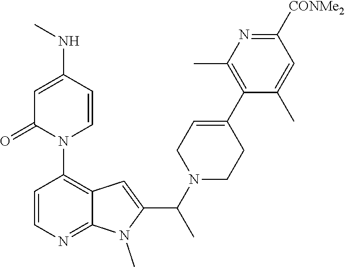

CuI (50 mg), Ints. 1Z78/1Z95 (30 mg/80 mg), and K₂CO₃ (0.1 g) were stirred 12 h in DMSO (5 mL) and trans-N,N′-dimethylcyclohexane-1,2-diamine (83 μL) at 100° C. under an inert atmosphere. The mixture was filtered and HPLC-purified to give Int. 1Z100 N,N,2,4-tetramethyl-1′-(1-(1-methyl-4-(4-(methylamino)-2-oxopyridin-1 (2H)-yl)-1H-pyrrolo[2,3-b]pyridin-2-yl)ethyl)-1′,2′,3′,6′-tetrahydro-[3,4′-bipyridine]-6-carboxamide (11 mg).

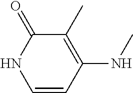

Paraformaldehyde (0.29 g) was added to a solution of Int. 1Z81 (0.40 g) in MeOH (10 mL) under an inert atmosphere. 30% NaOMe in MeOH (3 mL) was added drop-wise and stirring continued 4 h at 0-55° C. NaBH₄ (0.38 g) was added portion-wise and stirring continued 4 h at 0-55° C. The mixture was diluted with aq. citric acid, concentrated, and purified by FC on neutral alumina (DCM/MeOH 9:1) to give Int. 1Z104 3-methyl-4-(methylamino)pyridin-2 (1H)-one (0.30 g).

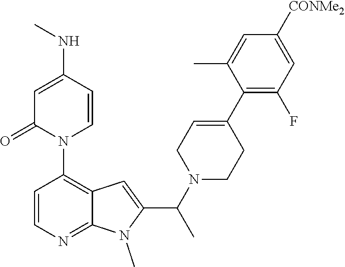

CuI (12 g), Ints. 2G81/1Z69 (8.6 g/32 g), and K₃PO₄ (40 g) were stirred 16 h in dioxane (375 mL) and trans-N,N′-dimethylcyclohexane-1,2-diamine (20 mL) at 107° C. under an inert atmosphere. The mixture was filtered. The OL (aq. NH₃/DCM/MeOH) was dried, filtered, and concentrated. The residue was refluxed 1 h in EtOAc/DCM (10:3) and filtered while hot, the filter was washed with EtOAc and Et₂O and the filtrate concentrated to give Int. 1Z105 3-fluoro-N,N,5-trimethyl-4-(1-(1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzamide (22 g).

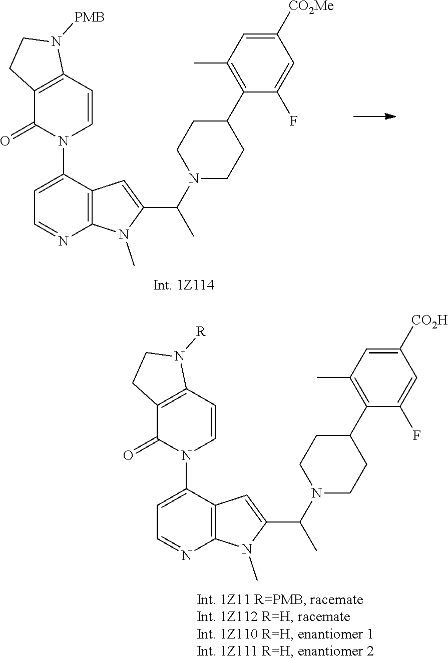

CuI (1.1 g), Ints. 3E181/HH10 (1.6 g/2.8 g), and K₃PO₄ (3.6 g) were stirred 16 h in dioxane (15 mL) and trans-N,N′-dimethylcyclohexane-1,2-diamine (1.8 mL) at 100° C. under an inert atmosphere. The mixture was filtered. The OL (aq. NH₃/DCM/MeOH) was dried, filtered, concentrated, and purified by FC (DCM/MeOH 19:1) to give Int. 1Z114 methyl 3-fluoro-4-(1-(1-(4-(1-(4-methoxybenzyl)-4-oxo-1,2,3,4-tetrahydro-5H-pyrrolo[3,2-c]pyridin-5-yl)-1-methyl-1H-pyrrolo[2,3-b]pyridin-2-yl)ethyl)piperidin-4-yl)-5-methylbenzoate (1.8 g). LiOH·H₂O (1.5 g) and Int. 1Z114 (4.6 g) were stirred 16 h in THF/MeOH/H₂O (3:1:2, 90 mL) at 0° C. to RT. The residue after concentration was stirred in aq. citric acid to precipitate Int. 1Z113 3-fluoro-4-(1-(1-(4-(1-(4-methoxybenzyl)-4-oxo-1,2,3,4-tetrahydro-5H-pyrrolo[3,2-c]pyridin-5-yl)-1-methyl-1H-pyrrolo[2,3-b]pyridin-2-yl)ethyl)piperidin-4-yl)-5-methylbenzoic acid (4.3 g). Int. 1Z113 (0.80 g) was stirred 16 h in TFA (7 mL) at 65° C. The mixture was concentrated and triturated in MTBE to give Int. 1Z112 3-fluoro-5-methyl-4-(1-(1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)piperidin-4-yl)benzoic acid (0.80 g, TFA salt). Int. 1Z112 (0.80 g) was resolved by SFC on a Sepiatc-200-001 instrument fitted with a (R,R) Whelk-01 250×30 mm 5 μm column operated at 30° C. and an eluent of 65% CO₂ and 35% 30 mM MeONH₃ in MeOH at a (90 g/min) and a back pressure of 120 bar to give Int. 1Z110 (0.30 g, first peak) and Int. 1Z111 (0.25 g, second peak) (S)-3-fluoro-5-methyl-4-(1-(1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)piperidin-4-yl)benzoic acid and (R)-3-fluoro-5-methyl-4-(1-(1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)piperidin-4-yl)benzoic acid. The absolute configurations of these compounds were not determined.

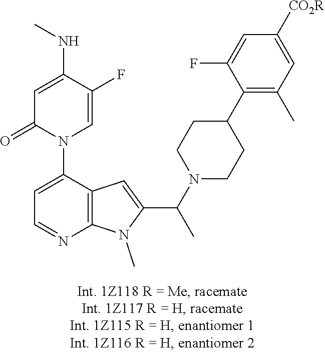

CuI (0.93 g), Ints. 2G68/2G85 (0.42 g/1.2 g), and K₃PO₄ (1.6 g) were stirred 16 h in dioxane (5 mL) and trans-N,N′-dimethylcyclohexane-1,2-diamine (0.78 mL) at 100° C. under an inert atmosphere. The OL (aq. NH₃/DCM/MeOH) was dried, filtered, concentrated, and purified by FC (DCM/MeOH 9:1) to give Int. 1Z118 methyl 3-fluoro-4-(1-(1-(4-(5-fluoro-4-(methylamino)-2-oxopyridin-1 (2H)-yl)-1-methyl-1H-pyrrolo[2,3-b]pyridin-2-yl)ethyl)piperidin-4-yl)-5-methylbenzoate (0.90 g). LiOH·H₂O (0.34 g) and Int. 1Z118 (0.90 g) were stirred 4 h in THF/MeOH/H₂O (2:1:1, 10 mL) at 0° C. to RT. The residue after concentration was stirred in aq. citric acid to precipitate Int. 1Z117 3-fluoro-4-(1-(1-(4-(5-fluoro-4-(methylamino)-2-oxopyridin-1 (2H)-yl)-1-methyl-1H-pyrrolo[2,3-b]pyridin-2-yl)ethyl)piperidin-4-yl)-5-methylbenzoic acid (0.50 g). Int. 1Z117 (0.60 g) was resolved by SFC on a Sepiatec-660 instrument fitted with a (R,R) Whelk-01 250×30 mm 5 μm column operated at 30° C. and an eluent of 55% CO₂ and 45% 30 mM MeONH₃ in MeOH at a (100 g/min) and a back pressure of 120 bar to give Int. 1Z115 (0.13 g, first peak) and Int. 1Z116 (0.14 g, second peak) (R)-3-fluoro-4-(1-(1-(4-(5-fluoro-4-(methylamino)-2-oxopyridin-1 (2H)-yl)-1-methyl-1H-pyrrolo[2,3-b]pyridin-2-yl)ethyl)piperidin-4-yl)-5-methylbenzoic acid and (S)-3-fluoro-4-(1-(1-(4-(5-fluoro-4-(methylamino)-2-oxopyridin-1 (2H)-yl)-1-methyl-1H-pyrrolo[2,3-b]pyridin-2-yl)ethyl)piperidin-4-yl)-5-methylbenzoic acid. The absolute configurations of these compounds were not determined.

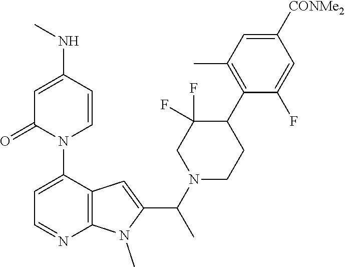

CuI (50 mg) and trans-N,N′-dimethylcyclohexane-1,2-diamine (82 μL) were added to a solution of Ints. 2G81/1Z134 (42 mg/0.14 g) and K₃PO₄ (0.17 g) in dioxane (10 mL) and stirred for 16 h at 105° C. under an inert atmosphere and filtered. The OL (aq. NH₃/DCM/MeOH) was dried, filtered and concentrated to give Int. 1Z135 4-(3,3-difluoro-1-(1-(1-methyl-4-(4-(methylamino)-2-oxopyridin-1 (2H)-yl)-1H-pyrrolo[2,3-b]pyridin-2-yl)ethyl)piperidin-4-yl)-3-fluoro-N,N,5-trimethylbenzamide (95 mg).

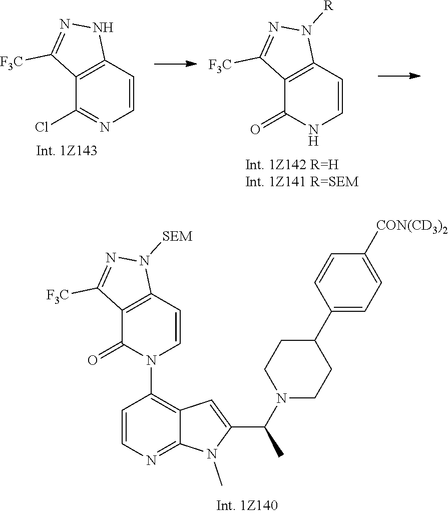

2.5M “BuLi in hexane (7 mL) was added to a solution of 2,4-dichloropyridine (2.0 g) in THF (135 mL) at −78° C. The mixture was stirred 0.5 h at −78° C. under an inert atmosphere. Ethyl 2,2,2-trifluoro-acetate (3.2 mL) was added drop-wise and stirring continued 0.75 h at −78° C. The OL (water/EtOAc) was dried, filtered and concentrated. The residue was stirred ON in THF (100 mL) and hydrazine hydrate (4.2 mL) at −40° C. to RT. The mixture was stirred ON min at −40° C. to RT. The residue after concentration was precipitated from MTBE to give Int. 1Z143 4-chloro-3-(trifluoromethyl)-1H-pyrazolo[4,3-c]-pyridine (1.1 g). Int. 1Z143 (1.1 g) was stirred 24 h in AcOH/H₂O (75:1, 15.2 mL) at 100° C. The mixture was concentrated. The OL (water/DCM) was dried, filtered, and concentrated to give Int. 1Z142 3-(trifluoromethyl)-1H-pyrazolo[4,3-c]pyridin-4-ol (0.96 g). SEM-C1 (0.84 mL) was added to a stirred mixture of Cs₂CO₃ (3.1 g) and Int. 1Z142 in DMF (7 mL). Stirring was continued 0.3 h. The OL (water/DCM) was dried, filtered, concentrated, and triturated in MTBE to give Int. 1Z141 3-(trifluoromethyl)-1-((2-(trimethylsilyl)ethoxy)methyl)-1H-pyrazolo[4,3-c]pyridin-4-ol (0.33 g). CuI (73 mg), Ints. 1Z141/1AF77 (96 mg/0.10 g), and K₃PO₄ (0.16 g) were stirred 2 h in DMF (10 mL) and trans-N,N′-dimethylcyclohexane-1,2-diamine (56 μL) at 120° C. under an inert atmosphere. The mixture was filtered. The OL (water/EtOAc) was concentrated and purified by FC (DCM/MeOH 9:1) to give Int. 1Z140 (S)—N,N-bis(methyl-d₃)-4-(1-(1-(1-methyl-4-(4-oxo-3-(trifluoromethyl)-1-((2-(trimethylsilyl)ethoxy)methyl)-1,4-dihydro-5H-pyrazolo[4,3-c]pyridin-5-yl)-1H-pyrrolo[2,3-b]pyridin-2-yl)ethyl)piperidin-4-yl)benzamide (0.10 g).

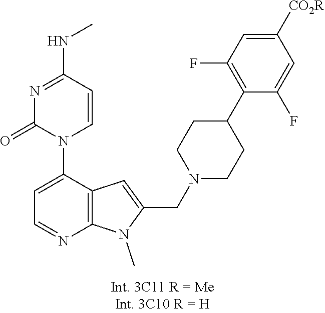

2-Chloro-5-fluoro-N-methylpyrimidin-4-amine (5 g) was stirred 16 h in water/formic acid (0.61 mL/23 mL) at 90° C. The mixture was diluted with EtOAc to precipitate Int. 3C12 (3.2 g). CuI (2.4 g), trans-N,N′-dimethylcyclohexane-1,2-diamine (2.0 mL), K₃PO₄ (4.0 g), Int. 3C12 (3 g), and 4-(methylamino)-pyrimidin-2 (1H)-one (1.0 g) were stirred 16 h in DMF/DMSO (5:1, 30 mL) at 110° C. under an inert atmosphere. The mixture was filtered. The OL (aq. NH₃/DCM/MEOH) was filtered, concentrated, and washed with Et₂O to give Int. 3C11 methyl 3,5-difluoro-4-(1-((1-methyl-4-(4-(methylamino)-2-oxopyrimidin-1 (2H)-yl)-1H-pyrrolo[2,3-b]pyridin-2-yl)methyl)piperidin-4-yl)benzoate (2.6 g). LiOH·H₂O (1.0 g) and Int. 3C11 (2.6 g) were stirred 4 h in THF/MeOH/H₂O (15:7:10, 32 mL) and concentrated. The AL (water/Et₂O) was diluted with aq. citric acid to precipitate Int. 3C10 3,5-difluoro-4-(1-((1-methyl-4-(4-(methylamino)-2-oxopyrimidin-1 (2H)-yl)-1H-pyrrolo[2,3-b]pyridin-2-yl)methyl)piperidin-4-yl)benzoic acid (2.4 g).

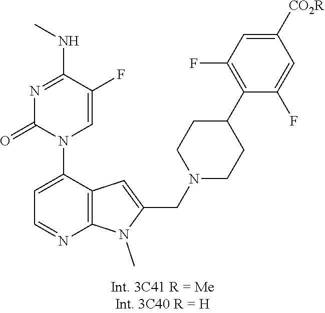

CuI (2.0 g), trans-N,N′-dimethylcyclohexane-1,2-diamine (1.7 mL), K₃PO₄ (3.3 g), and Ints. 3C12/3C15 (0.97 g/2.5 g) were stirred 12 h in dioxane (15 mL) at 100° C. under an inert atmosphere. The mixture was filtered. The OL (water/EtOAc) was concentrated and purified by FC (PE/EtOAc 4:1 to 3:17) to give Int. 3C41 methyl 3,5-difluoro-4-(1-((4-(5-fluoro-4-(methylamino)-2-oxopyrimidin-1 (2H)-yl)-1-methyl-1H-pyrrolo[2,3-b]pyridin-2-yl)methyl)piperidin-4-yl)benzoate (1.6 g). LiOH·H₂O (0.28 g) and Int. 3C41 (1.2 g) were stirred ON in THF/MeOH/H₂O (5:5:2, 24 mL). The residue after concentration was stirred in aq. citric acid to precipitate Int. 3C40 3,5-difluoro-4-(1-((4-(5-fluoro-4-(methylamino)-2-oxopyrimidin-1 (2H)-yl)-1-methyl-1H-pyrrolo[2,3-b]pyridin-2-yl)methyl)piperidin-4-yl)benzoic acid (0.90 g).

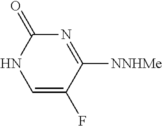

2-Chloro-5-fluoro-N-methylpyrimidin-4-amine (5 g) was stirred 16 h in water/formic acid (0.61 mL/23 mL) at 90° C. The mixture was diluted with EtOAc to precipitate Int. 3C12 5-fluoro-4-(methylamino)-pyrimidin-2 (1H)-one (3.2 g).

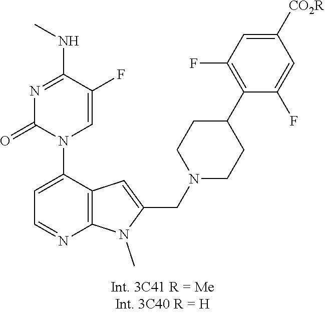

CuI (2.0 g) and trans-N,N′-dimethylcyclohexane-1,2-diamine (1.7 mL), K₃PO₄ (3.3 g), and Ints. 3C42/3C15 (0.97 g/2.5 g) were stirred 12 h in dioxane (15 mL) at 100° C. under an inert atmosphere. The mixture was filtered and the OL (water/EtOAc) was concentrated and purified by FC (PE/EtOAc 4:1 to 3:17) to give Int. 3C41 methyl 3,5-difluoro-4-(1-((4-(5-fluoro-4-(methylamino)-2-oxopyrimidin-1 (2H)-yl)-1-methyl-1H-pyrrolo[2,3-b]pyridin-2-yl)methyl)piperidin-4-yl)benzoate (1.6 g). LiOH·H₂O (0.28 g) and Int. 3C41 (1.2 g) were stirred ON in THF/MeOH/H₂O (5:5:2, 24 mL). The solid residue after concentration was stirred in water (pH adjusted with aq. citric acid), filtered and dried to give Int. 3C40 3,5-difluoro-4-(1-((4-(5-fluoro-4-(methylamino)-2-oxopyrimidin-1 (2H)-yl)-1-methyl-1H-pyrrolo[2,3-b]pyridin-2-yl)methyl)piperidin-4-yl)benzoic acid (0.90 g).

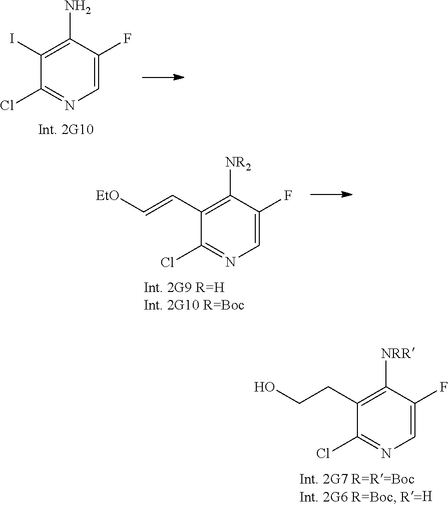

NaOAc (13 g), ICl (20 g), and 2-chloro-5-fluoropyridin-4-amine (12 g) were stirred 48 h in AcOH (100 mL). The mixture was concentrated. The OL (sat. aq. NaHCO₃/EtOAc) was dried, filtered, concentrated, and purified by FC (hexane/EtOAc 4:1) to give Int. 2G10 2-chloro-5-fluoro-3-iodopyridin-4-amine (12.5 g). Int. 2G10 (11.5 g), K₃PO₄ (18 g), trans-2-ethoxyvinylboronic acid pinacol ester (10 g), Pd(OAc)₂ (0.28 g), and SPhos (1.3 g) were stirred 16 h in ACN/H₂O (3:2, 100 mL) at 110° C. under an inert atmosphere. The mixture was filtered. The OL (water/EtOAc) was dried, filtered, concentrated, and purified by FC (hexane/EtOAc 3:2) to give Int. 2G9 (E)-2-chloro-3-(2-ethoxyvinyl)-5-fluoropyridin-4-amine (6 g). Boc₂O (4.5 g), DMAP (0.17 g), and Int. 2G9 (3 g) were stirred 24 h in DCM/Et₃N (5:2, 14 mL). The OL (water/DCM) was dried, concentrated, and purified by FC (hexane/EtOAc 19:1) to give Int. 2G8 tert-butyl (E)-(tert-butoxycarbonyl)(2-chloro-3-(2-ethoxyvinyl)-5-fluoropyridin-4-yl)carbamate (4.5 g). Hg (OAc)₂ (5.1 g) in H₂O (100 mL) and Int. 2G8 (4.5 g) were 0.5 h in THF (100 mL) at 0° C. NaBH₄ (2.1 g) in aq. K₂CO₃ (50 mL) was added. The mixture was stirred 16 h at 0° C. to RT. The OL (water/EtOAc) was dried, filtered, concentrated, and purified by FC (hexane/EtOAc 1:0 to 4:1) to give Int. 2G7 tert-butyl (tert-butoxycarbonyl)(2-chloro-5-fluoro-3-(2-hydroxyethyl)pyridin-4-yl)carbamate (3.2 g). Int. 2G7 (3.2 g) and K₂CO₃ (5.6 g) were stirred 16 h in MeOH (50 mL) and concentrated. The OL (sat. aq. NaHCO₃/EtOAc/DCM) was concentrated to give Int. 2G6 tert-butyl (2-chloro-5-fluoro-3-(2-hydroxyethyl)pyridin-4-yl)carbamate (2 g).

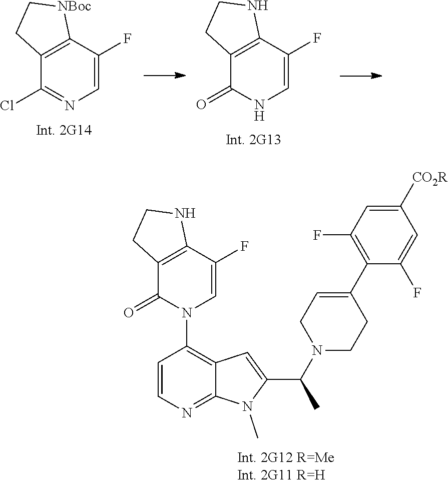

Int. 2G6 (2 g) was stirred 16 h in DCM (20 mL) and MsCl (0.80 mL) at 0° C. to RT. Another portion of MsCl (2.7 mL) was added and stirring was continued 5 h at 50° C. The OL (water/DCM) was dried, filtered, concentrated, and purified by FC (hexane/EtOAc 4:1) to give Int. 2G14 tert-butyl 4-chloro-7-fluoro-2,3-dihydro-1H-pyrrolo[3,2-c]pyridine-1-carboxylate (1.3 g). Int. 2G14 (1 g), Pd₂(dba)₃ (0.17 g), and BippyPhos (0.18 g) were stirred 16 h in dioxane (10 mL) and CsOH (50% in water, 5 mL) 105° C. The mixture was filtered. The residue after concentration was stirred in aq. citric acid to precipitate a solid that was purified by FC (DCM/MeOH 17:3) to give Int. 2G13 7-fluoro-1,2,3,5-tetrahydro-4H-pyrrolo[3,2-c]pyridin-4-one (0.30 g). CuI (1.6 g), Ints. 2G13/HH5-2 (0.69 g/2.0 g), and Cs₂CO₃ (4.0 g) were stirred 12 h in dioxane (30 mL) and 1,2-cyclohexanediamine (1.3 mL) at 110° C. under an inert atmosphere. The mixture was filtered. The OL (aq. NH₃/EtOAc) was dried, concentrated, and purified by FC (DCM/MeOH 1:0 to 9:1) to give Int. 2G12 methyl (S)-3,5-difluoro-4-(1-(1-(4-(7-fluoro-4-oxo-1,2,3,4-tetrahydro-5H-pyrrolo[3,2-c]-pyridin-5-yl)-1-methyl-1H-pyrrolo[2,3-b]pyridin-2-yl)ethyl)-1,2,3,6-tetrahydropyridin-4-yl)benzoate (1.4 g). LiOH·H₂O (0.43 g) and Int. 2G12 (1.15 g) were stirred 12 h in THF/MeOH/H₂O (2:2:1, 20 mL) at 0° C. to RT. The residue after concentration was stirred in aq. citric acid to precipitate Int. 2G11 (S)-3,5-difluoro-4-(1-(1-(4-(7-fluoro-4-oxo-1,2,3,4-tetrahydro-5H-pyrrolo[3,2-c]pyridin-5-yl)-1-methyl-1H-pyrrolo[2,3-b]pyridin-2-yl)ethyl)-1,2,3,6-tetrahydropyridin-4-yl)benzoic acid (0.95 g).

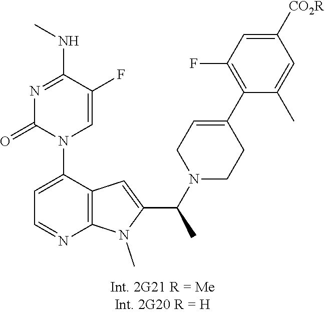

2-Chloro-5-fluoro-N-methylpyrimidin-4-amine (5 g) was stirred 16 h in water/formic acid (0.61 mL/23 mL) at 90° C. The mixture was diluted with EtOAc to precipitate Int. 3C12 (3.2 g). CuI (1.6 g), K₃PO₄ (2.6 g), and Ints. 3C12/1AF86 (0.77 g/2 g) were stirred 12 h in dioxane (25 mL) and 1,2-cyclohexanediamine (1.3 mL) at 100° C. under an inert atmosphere. The mixture was filtered. The OL (water/EtOAc) was concentrated, and purified by FC (PE/EtOAc 1:4 to 3:17) to give Int. 2G21 methyl (S)-3-fluoro-4-(1-(1-(4-(5-fluoro-4-(methylamino)-2-oxopyrimidin-1 (2H)-yl)-1-methyl-1H-pyrrolo[2,3-b]pyridin-2-yl)ethyl)-1,2,3,6-tetrahydropyridin-4-yl)-5-methylbenzoate (0.95 g). LiOH·H₂O (0.16 g) and Int. 2G21 (0.70 g) were stirred 16 h in THF/MeOH/H₂O (3:3:1, 23 mL). The residue after concentration was stirred in aq. citric acid to precipitate Int. 2G20 (S)-3-fluoro-4-(1-(1-(4-(5-fluoro-4-(methylamino)-2-oxopyrimidin-1 (2H)-yl)-1-methyl-1H-pyrrolo[2,3-b]pyridin-2-yl)ethyl)-1,2,3,6-tetrahydropyridin-4-yl)-5-methylbenzoic acid (0.40 g).

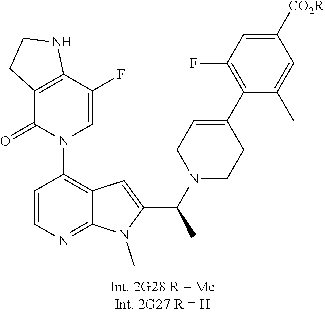

CuI (0.49 g), Cs₂CO₃ (6.7 g), and Ints. 2G13/1AF86 (1.0 g/2.5 g) were stirred 16 h in dioxane (25 mL) and 1,2-cyclohexanediamine (0.81 mL) at 90° C. under an inert atmosphere. The mixture was filtered. The OL (aq. NH₃/EtOAc) was dried, filtered, concentrated, and purified by FC (DCM/MeOH 19:1) to give Int. 2G28 methyl (S)-3-fluoro-4-(1-(1-(4-(7-fluoro-4-oxo-1,2,3,4-tetrahydro-5H-pyrrolo[3,2-c]-pyridin-5-yl)-1-methyl-1H-pyrrolo[2,3-b]pyridin-2-yl)ethyl)-1,2,3,6-tetrahydropyridin-4-yl)-5-methyl-benzoate (1 g). LiOH·H₂O (0.24 g) and Int. 2G28 (0.80 g) were stirred 48 h in THF/H₂O (1:1, 25 mL) at 0° C. to RT. The residue after concentration was stirred in aq. citric acid to precipitate Int. 2G27 (S)-3-fluoro-4-(1-(1-(4-(7-fluoro-4-oxo-1,2,3,4-tetrahydro-5H-pyrrolo[3,2-c]pyridin-5-yl)-1-methyl-1H-pyrrolo[2,3-b]pyridin-2-yl)ethyl)-1,2,3,6-tetrahydropyridin-4-yl)-5-methylbenzoic acid (0.55 g).

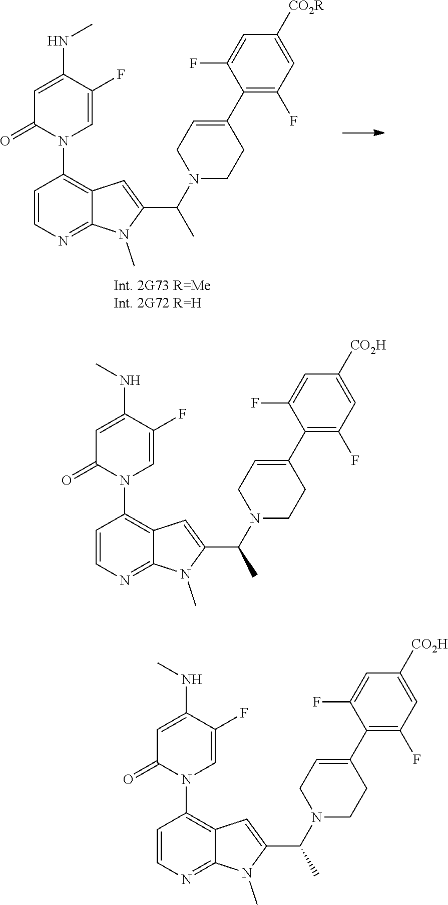

Int. 2G73 methyl 3,5-difluoro-4-(1-(1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)benzoate (6.3 g) was prepared similarly to Int. 2G67 from Ints. 2G68/3C26 (2.4 g/7.5 g). LiOH (2.3 g) and Int. 2G73 (6.0 g) were stirred 16 h in THF/MeOH/water (2:1:2, 80 mL). The residue after concentration was stirred in 10% aq. citric acid to precipitate Int. 2G72 3,5-difluoro-4-(1-(1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)benzoic acid (5.4 g). Int. 2G72 (5.35 g) was resolved by SFC on a SFC-150-009 instrument fitted with a (R,R) Whelk 250×30 mm 25 μm column operated at 30° C. and an eluent of 50% CO₂ and 50% MeOH containing 0.5% MeONH₃ at a (100 g/min) and a back pressure of 100 bar to give Int. 2G43 (2.1 g, first peak) and Int. 2G44 (2.5 g, second peak) (R)-3,5-difluoro-4-(1-(1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)benzoic acid and (S)-3,5-difluoro-4-(1-(1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)benzoic acid. The absolute configurations of these compounds were not determined.

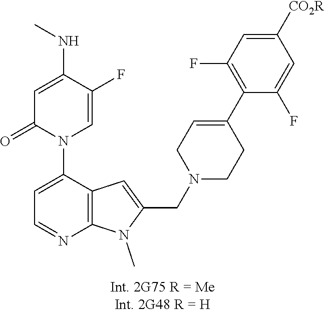

Int. 2G75 methyl 3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)-1,2,3,6-tetrahydropyridin-4-yl)benzoate (4.8 g) was prepared similarly to Int. 2G67 from Ints. 2G68/3C31 (2.9 g/7.5 g). LiOH·H₂O (1.8 g) and Int. GG9 (4.8 g) were stirred 4 h in THF/MeOH/H₂O (8:5:3, 80 mL). The mixture was concentrated. The AL (water/Et₂O) was diluted with aq. citric acid to precipitate Int. 2G48 3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)-1,2,3,6-tetrahydropyridin-4-yl)benzoic acid (4.0 g).

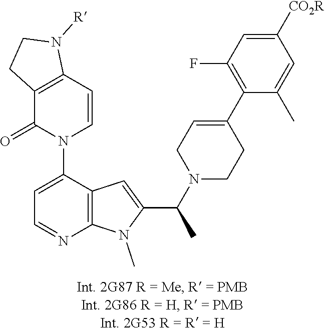

CuI (1.6 g), K₃PO₄ (5.2 g), and Ints. 1AF86/3E181 (4.0 g/2.3 g) were stirred 16 h in dioxane (60 mL) and 1,2-cyclohexanediamine (2.6 mL) at 100° C. under an inert atmosphere. The mixture was filtered. The OL (water/DCM/MeOH) was dried, concentrated, and purified by FC (DCM/MeOH 19:1 to 9:1) to give Int. 2G87 methyl (S)-3-fluoro-4-(1-(1-(4-(1-(4-methoxybenzyl)-4-oxo-1,2,3,4-tetrahydro-5H-pyrrolo[3,2-c]pyridin-5-yl)-1-methyl-1H-pyrrolo[2,3-b]pyridin-2-yl)ethyl)-1,2,3,6-tetrahydropyridin-4-yl)-5-methylbenzoate (4.5 g). LiOH·H₂O (0.46 g) and Int. 2G87 (4.5 g) were stirred 4 h in THF/MeOH/H₂O (5:2:1, 42 mL). The residue after concentration was stirred in water at pH 5-7 (adjusted with citric acid), filtered and dried to give Int. 2G86 (S)-3-fluoro-4-(1-(1-(4-(1-(4-methoxybenzyl)-4-oxo-1,2,3,4-tetrahydro-5H-pyrrolo[3,2-c]pyridin-5-yl)-1-methyl-1H-pyrrolo[2,3-b]pyridin-2-yl)ethyl)-1,2,3,6-tetrahydropyridin-4-yl)-5-methylbenzoic acid (4.0 g). Int. 2G86 (4.0 g) was stirred 16 h in TFA (40 mL) at 80° C., concentrated, and washed with Et₂O and hexane to give Int. 2G53 (S)-3-fluoro-5-methyl-4-(1-(1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzoic acid (3.4 g, TFA salt).

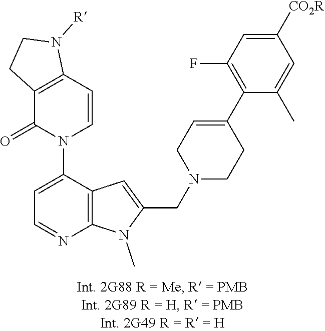

Int. 3E181 methyl 3-fluoro-4-(1-((4-(1-(4-methoxybenzyl)-4-oxo-1,2,3,4-tetrahydro-5H-pyrrolo[3,2-c]pyridin-5-yl)-1-methyl-1H-pyrrolo[2,3-b]pyridin-2-yl)methyl)-1,2,3,6-tetrahydropyridin-4-yl)-5-methylbenzoate (1.2 g) was prepared similarly to Int. 2G67 from Ints. 3E181/3C73 (0.78 g/1.3 g). LiOH·H₂O (0.39 g) and Int. 2G88 (1.2 g) were stirred 5 h in THF/MeOH/H₂O (2:1:1, 10 mL) at 0° C. to RT. The residue after concentration was stirred in 10% aq. citric acid to precipitate Int. 2G89 3-fluoro-4-(1-((4-(1-(4-methoxybenzyl)-4-oxo-1,2,3,4-tetrahydro-5H-pyrrolo[3,2-c]pyridin-5-yl)-1-methyl-1H-pyrrolo[2,3-b]pyridin-2-yl)methyl)-1,2,3,6-tetrahydropyridin-4-yl)-5-methylbenzoic acid (1.1 g). Int. 2G89 (1.2 g) was stirred 16 h in TFA (18 mL) at 70° C., concentrated, and washed with Et₂O and pentane to give Int. 2G49 3-fluoro-5-methyl-4-(1-((1-methyl-4-(4-oxo-1,2,3,4-tetrahydro-5H-pyrrolo[3,2-c]pyridin-5-yl)-1H-pyrrolo[2,3-b]pyridin-2-yl)methyl)-1,2,3,6-tetrahydropyridin-4-yl)-benzoic acid (1.0 g, TFA salt).

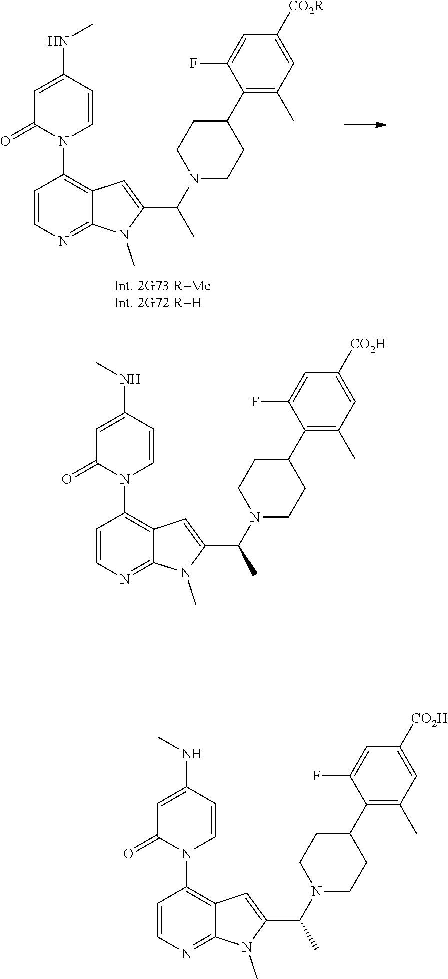

Int. 2G84 methyl 3-fluoro-5-methyl-4-(1-(1-(1-methyl-4-(4-(methylamino)-2-oxopyridin-1 (2H)-yl)-1H-pyrrolo[2,3-b]pyridin-2-yl)ethyl)piperidin-4-yl)benzoate (0.80 g) was prepared similarly to Int. 2G67 from Ints. 2G81/2G85 (0.50 g/1.5 g). LiOH·H₂O (0.40 g) and Int. 2G84 (1.0 g) were stirred 16 h in THF/MeOH/H₂O (2:1:1, 24 mL) at 0° C. to RT. The residue after concentration was stirred in 3% aq. citric acid, filtered, and dried to give Int. 2G83 3-fluoro-5-methyl-4-(1-(1-(1-methyl-4-(4-(methylamino)-2-oxopyridin-1 (2H)-yl)-1H-pyrrolo[2,3-b]pyridin-2-yl)ethyl)piperidin-4-yl)benzoic acid (0.73 g). Int. 2G83 (0.73 g) was resolved by SFC on a SFC-150-009 instrument fitted with a (R,R) Whelk-01 250×30 mm 5 μm column operated at 30° C. and an eluent of 60% CO₂ and 40% MeOH containing 0.5% MeONH₃ at a (90 g/min) and a back pressure of 100 bar to give Int. 2G55 (0.21 g, first peak) and Int. 2G54 (0.22 g, second peak) (R)-3-fluoro-5-methyl-4-(1-(1-(1-methyl-4-(4-(methylamino)-2-oxopyridin-1 (2H)-yl)-1H-pyrrolo[2,3-b]pyridin-2-yl)ethyl)piperidin-4-yl)benzoic acid and (S)-3-fluoro-5-methyl-4-(1-(1-(1-methyl-4-(4-(methylamino)-2-oxopyridin-1 (2H)-yl)-1H-pyrrolo[2,3-b]pyridin-2-yl)ethyl)piperidin-4-yl)benzoic acid. The absolute configurations were not determined for these compounds.

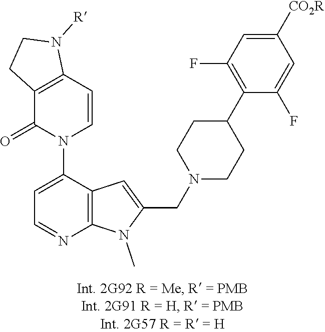

CuI (0.80 g), K₃PO₄ (2.7 g), and Ints. 3E181/3C15 (1.2 g/2.0 g) were stirred 16 h in dioxane (20 mL) and 1,2-cyclohexanediamine (1.3 mL) at 90° C. under an inert atmosphere. The OL (aq. NH₃/EtOAc) was dried, filtered, concentrated, and purified by FC (DCM/MeOH 9:1) to give Int. 2G92 methyl 3,5-difluoro-4-(1-((4-(1-(4-methoxybenzyl)-4-oxo-1,2,3,4-tetrahydro-5H-pyrrolo[3,2-c]pyridin-5-yl)-1-methyl-1H-pyrrolo[2,3-b]pyridin-2-yl)methyl)piperidin-4-yl)benzoate (1.8 g). LiOH·H₂O (0.58 g) and Int. 2G92 (1.8 g) were stirred 16 h in THF/MeOH/H₂O (2:1:1, 40 mL) at 0° C. to RT. The residue after concentration was stirred in water and diluted with aq. citric acid to precipitate Int. 2G91 3,5-difluoro-4-(1-((4-(1-(4-methoxybenzyl)-4-oxo-1,2,3,4-tetrahydro-5H-pyrrolo[3,2-c]pyridin-5-yl)-1-methyl-1H-pyrrolo[2,3-b]pyridin-2-yl)methyl)piperidin-4-yl)benzoic acid (1.5 g). Int. 2G91 (1.5 g) was stirred 16 h in TFA (30 mL) at 0° C. to 70° C., concentrated, and washed with Et₂O to give crude Int. 2G57 3,5-difluoro-4-(1-((1-methyl-4-(4-oxo-1,2,3,4-tetrahydro-5H-pyrrolo[3,2-c]pyridin-5-yl)-1H-pyrrolo[2,3-b]pyridin-2-yl)methyl)piperidin-4-yl)benzoic acid (1.1 g, TFA salt).

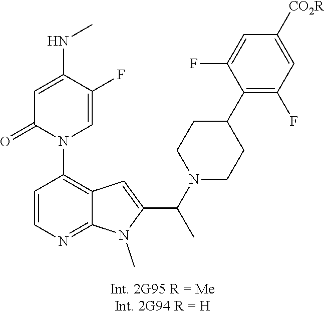

CuI (6.2 g), K₃PO₄ (10 g), and Ints. 2G68/2G96 (3.0 g/8.0 g) were stirred 16 h in dioxane (250 mL) and 1,2-cyclohexanediamine (5 mL) at 110° C. under an inert atmosphere. The mixture was filtered. The OL (aq. NH₃/DCM/MeOH) was dried, concentrated, and washed with Et₂O to give Int. 2G95 methyl 3,5-difluoro-4-(1-(1-(4-(5-fluoro-4-(methylamino)-2-oxopyridin-1 (2H)-yl)-1-methyl-1H-pyrrolo[2,3-b]pyridin-2-yl)ethyl)piperidin-4-yl)benzoate (6 g). LiOH·H₂O (1.4 g) and Int. 2G95 (6 g) were stirred 4 h in THF/MeOH/H₂O (3:2:1, 120 mL) and concentrated. The AL (water/Et₂O) was diluted with aq. citric acid to precipitate Int. 2G94 3,5-difluoro-4-(1-(1-(4-(5-fluoro-4-(methylamino)-2-oxopyridin-1 (2H)-yl)-1-methyl-1H-pyrrolo[2,3-b]pyridin-2-yl)ethyl)piperidin-4-yl)benzoic acid (5.5 g).

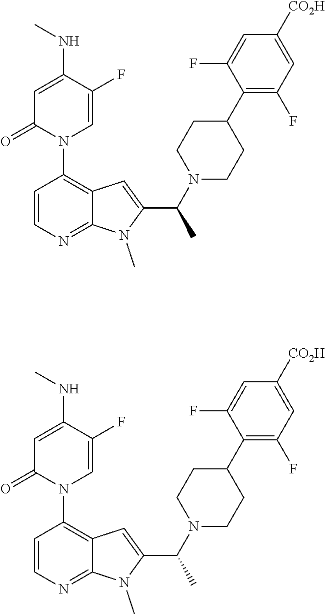 [SMILES: CNc1cc(=O)n(-c2ccnc3c2cc([C@H](C)N2CCC(c4c(F)cc(C(=O)O)cc4F)CC2)n3C)cc1F ; CNc1cc(=O)n(-c2ccnc3c2cc([C@@H](C)N2CCC(c4c(F)cc(C(=O)O)cc4F)CC2)n3C)cc1F]

Int. 2G94 (5.5 g) was resolved by SFC on a SFC-150-022 instrument fitted with a (R,R) WHELK-1 250×30 mm 5 μm column operated at 30° C. and an eluent of 55% CO₂ and 45% MeOH containing 0.5% MeONH₃ at a (100 g/min) and a back pressure of 100 bar to give Int. 2G93 (2.0 g, first peak) and Int. 2G50 (2.0 g, second peak) (S)-3,5-difluoro-4-(1-(1-(4-(5-fluoro-4-(methylamino)-2-oxopyridin-1 (2H)-yl)-1-methyl-1H-pyrrolo[2,3-b]pyridin-2-yl)ethyl)piperidin-4-yl)benzoic acid and (R)-3,5-difluoro-4-(1-(1-(4-(5-fluoro-4-(methylamino)-2-oxopyridin-1 (2H)-yl)-1-methyl-1H-pyrrolo[2,3-b]pyridin-2-yl)ethyl)piperidin-4-yl)benzoic acid. The absolute configurations were not determined for these compounds.

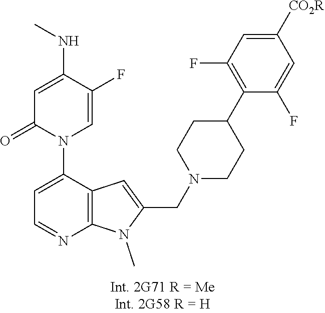

Int. 2G71 methyl 3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoate (6.5 g) was prepared similarly to Int. 2G67 from Ints. 2G68/3C15 (3.6 g/10 g). LiOH·H₂O (2.5 g) was added to a solution of Int. GG4 (6.5 g) in THF/MeOH/H₂O (8:5:3, 80 mL) and stirred for 4 h at RT and concentrated. The AL (water/Et₂O) was diluted with aq. citric acid to precipitate Int. 2G58 3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoic acid (6.0 g).

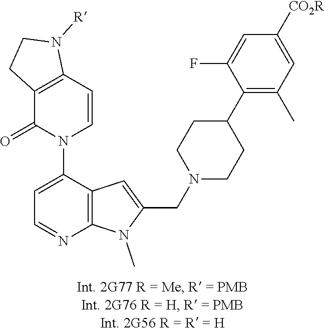

CuI (1.6 g), K₃PO₄ (2.7 g), and Ints. 3E181/HH11 (1.2 g/2.0 g) were stirred 16 h in dioxane (20 mL) and 1,2-cyclohexanediamine (1.3 mL) at 120° C. under an inert atmosphere. The OL (water/EtOAc) was dried, concentrated, and purified by FC (DCM/MeOH 9:1) to give Int. 2G77 methyl 3-fluoro-4-(1-((4-(1-(4-methoxybenzyl)-4-oxo-1,2,3,4-tetrahydro-5H-pyrrolo[3,2-c]pyridin-5-yl)-1-methyl-1H-pyrrolo[2,3-b]pyridin-2-yl)methyl)piperidin-4-yl)-5-methylbenzoate (2.4 g). LiOH·H₂O (0.78 g) and Int. 2G77 (2.4 g) were stirred 16 h in THF/MeOH/H₂O (2:1:1, 40 mL) at 0° C. to RT. The residue after concentration was stirred in water and diluted with aq. citric acid to precipitate Int. 2G76 3-fluoro-4-(1-((4-(1-(4-methoxybenzyl)-4-oxo-1,2,3,4-tetrahydro-5H-pyrrolo[3,2-c]pyridin-5-yl)-1-methyl-1H-pyrrolo[2,3-b]pyridin-2-yl)methyl)piperidin-4-yl)-5-methylbenzoic acid (2.1 g). Int. 2G76 (1.2 g) was stirred 16 h in TFA (30 mL) at 70° C., concentrated, and washed with Et₂O and pentane to give Int. 2G56 3-fluoro-5-methyl-4-(1-((1-methyl-4-(4-oxo-1,2,3,4-tetrahydro-5H-pyrrolo[3,2-c]pyridin-5-yl)-1H-pyrrolo[2,3-b]pyridin-2-yl)methyl)piperidin-4-yl)benzoic acid (0.90 g, TFA salt).

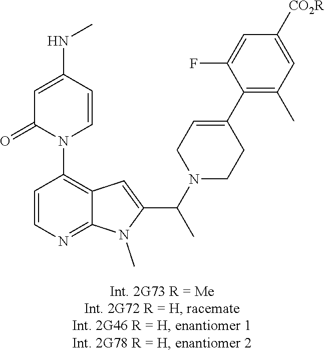

Paraformaldehyde (41 g) and 4-aminopyridin-2 (1H)-one (50 g) were stirred 4 h in MeOH (200 mL) and NaOMe (30% in MeOH, 0.42 L) at 0° C. to 55° C. NaBH₄ (53 g) was and the mixture stirred 4 h at 0° C. to 55° C. The reaction mixture was diluted with aq. citric acid, concentrated, and washed with DCM/MeOH (3:2). The filtrate was dried, concentrated and purified by FC on neutral alumina (DCM/MeOH 5:1) to give Int. 2G81 4-(methylamino)pyridin-2 (1H)-one (23 g). Int. 2G80 methyl 3-fluoro-5-methyl-4-(1-(1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoate (40.0 g) was prepared similarly to Int. 2G67 from Ints. 2G81/1AF87 (13.8 g/45.0 g). LiOH·H₂O (16 g) and Int. 2G80 (40 g) were stirred 16 h in THF/MeOH/H₂O (4:3:3, 150 mL) at 0° C. to RT. The residue after concentration was stirred in water and diluted with aq. citric acid to precipitate Int. 2G79 3-fluoro-5-methyl-4-(1-(1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoic acid (38 g). Int. 2G79 (38 g) was resolved by SFC on a SEPIATEC-250 instrument fitted with a (R,R) WHELK PACKED 250×30 mm 5 μm column operated at 30° C. and an eluent of 70% CO₂ and 30% MeOH containing 0.5% MeONH₃ at a (100 g/min) and a back pressure of 120 bar to give Int. 2G46 (12 g, first peak) and Int. 2G78 (11 g, second peak) (R)-3-fluoro-5-methyl-4-(1-(1-(1-methyl-4-(4-(methylamino)-2-oxopyridin-1 (2H)-yl)-1H-pyrrolo[2,3-b]pyridin-2-yl)ethyl)-1,2,3,6-tetrahydro-pyridin-4-yl)benzoic acid and (S)-3-fluoro-5-methyl-4-(1-(1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoic acid. The absolute configurations were not determined for these compounds.

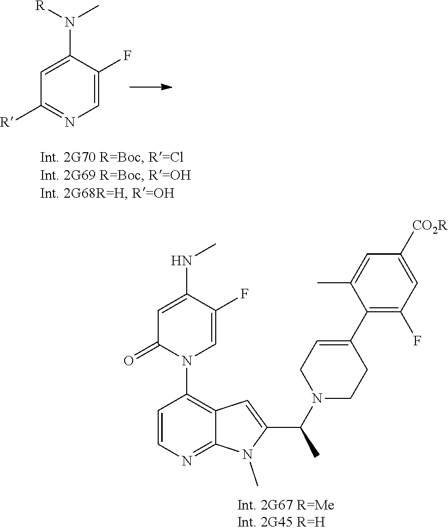

tert-Butyl (2-chloro-5-fluoropyridin-4-yl)carbamate (50.0 g) was added to a solution of NaH (5.8 g) in DMF (500 mL) at 0° C. and stirred for 1 h at RT. MeI (25 mL) was added and the mixture stirred for further 4 h. The OL (water/EtOAc) was dried, filtered, concentrated, and purified by FC (DCM/MeOH 1:0 to 19:1) to give Int. 2G70 tert-butyl (2-chloro-5-fluoropyridin-4-yl)(methyl)carbamate (45.0 g). Int. 2G70 (45.0 g), KOH (38.7 g), Pd₂(dba)₃ (15.8 g), and tert-butyl-Xphos (14.7 g) were stirred 16 h in dioxane/H₂O (4:1, 500 mL) at 110° C. under an inert atmosphere. The AL (water/DCM) was diluted with 10% aq. citric acid to precipitate Int. 2G69 tert-butyl (5-fluoro-2-oxo-1,2-dihydropyridin-4-yl)-(methyl)carbamate (21.0 g). Int. 2G69 (10.0 g) was stirred 16 h in DCM/4M HCl in dioxane (5:6, 110 mL) at 0° C. to RT. The residue after concentrated was washed with Et₂O to give Int. 2G68 5-fluoro-4-(methylamino)pyridin-2 (1H)-one (4.7 g, HCl salt). CuI (2.4 g), K₃PO₄ (8.2 g), and Ints. 2G68/1AF86 (2.8 g/6.2 g) were stirred 16 h in dioxane (80 mL) and 1,2-cyclohexanediamine (4.1 mL) at 100° C. under an inert atmosphere of argon. The mixture was filtered. The OL (aq. NH₃/DCM/MeOH) was dried, concentrated, and triturated in Et₂O/EtOAc (4:1) to give Int. 2G67 methyl (S)-3-fluoro-4-(1-(1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoate (5.6 g). LiOH (1.7 g) and Int. 2G67 (5.5 g) were stirred 16 h in H₂O/MeOH (20 mL/10 mL). The residue after concentration was stirred in 10% aq. citric acid to precipitate Int. 2G45 (S)-3-fluoro-4-(1-(1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoic acid (5.0 g).

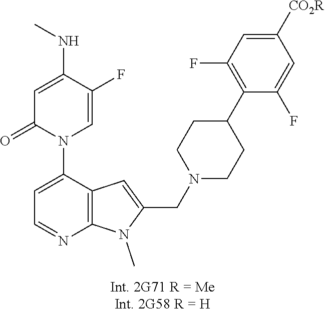

Int. 2G71 methyl 3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoate (6.5 g) was prepared similarly to Int. 2G67 from Ints. 2G68/3C15 (3.6 g/10 g). LiOH·H₂O (2.5 g) was added to a solution of Int. GG4 (6.5 g) in THF/MeOH/H₂O (8:5:3, 80 mL) and stirred for 4 h at RT and concentrated. The AL (water/Et₂O) was dilute with aq. citric acid to precipitate Int. 2G58 3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoic acid (6.0 g).

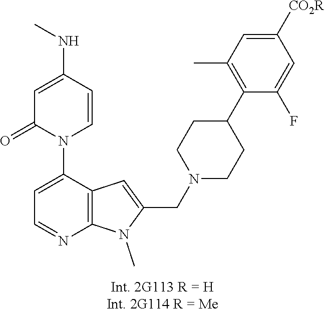

Int. 2G114 methyl 3-fluoro-5-methyl-4-(1-((1-methyl-4-(4-(methylamino)-2-oxopyridin-1 (2H)-yl)-1H-pyrrolo[2,3-b]pyridin-2-yl)methyl)piperidin-4-yl)benzoate (2.0 g) was prepared similarly to Int. GG1 from Ints. 2G81/2G115 (1.3 g/3.5 g). LiOH·H₂O (0.79 g) and Int. 2G114 (2.0 g) were stirred 16 h in THF/MeOH/H₂O (3:1:2, 30 mL) at 0° C. to RT. The residue after concentration was stirred in aq. citric acid to precipitate Int. 2G113 3-fluoro-5-methyl-4-(1-((1-methyl-4-(4-(methylamino)-2-oxopyridin-1 (2H)-yl)-1H-pyrrolo[2,3-b]pyridin-2-yl)methyl)piperidin-4-yl)benzoic acid (1.7 g).

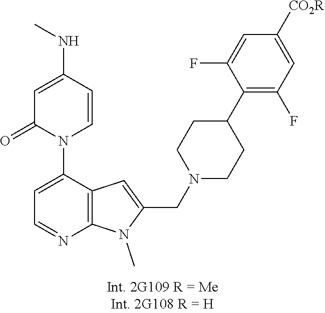

CuI (1.6 g), Ints. 2G81/3C15 (0.62 g/2.0 g), and K₃PO₄ (2.7 g) were stirred 12 h in dioxane (70 mL) and 1,2-cyclohexanediamine (1.0 mL) at 110° C. under an inert atmosphere. The mixture was filtered. The OL (water/DCM/MeOH) was dried, concentrated, and washed with Et₂O to give Int. 2G109 methyl 3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoate (1.5 g). LiOH·H₂O (39 mg) and Int. GG2 (100 mg) were stirred 16 h in THF/MeOH/H₂O (2:2:1, 2.5 mL) at 0° C. to RT. The residue after concentration was stirred in aq. HCl to precipitate Int. 2G108 3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoic acid (80 mg).

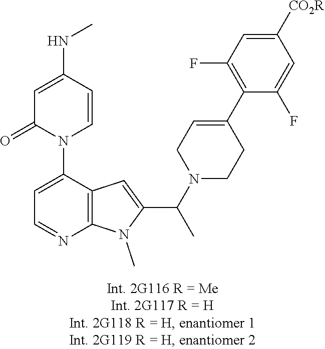

CuI (8.5 g), Ints. 2G81/3C26 (3.3 g/11 g), and K₂CO₃ (14 g) were stirred 12 h in dioxane (150 mL) and trans-N,N′-dimethylcyclohexane-1,2-diamine (7 mL) at 110° C. under an atmosphere of nitrogen. The mixture was filtered. The OL (water/EtOAc) was dried, filtered, concentrated, and reprecipitated from EtOAc/DCM to give Int. 2G116 methyl 3,5-difluoro-4-(1-(1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoate (5.5 g). LiOH·H₂O (2.2 g) and Int. 2G116 (5.5 g) were stirred 6 h in THF/MeOH/H₂O (2:2:1, 100 mL) at 0-60° C. The residue after concentration was stirred in aq. citric acid to precipitate Int. 2G117 3,5-difluoro-4-(1-(1-(1-methyl-4-(4-(methylamino)-2-oxopyridin-1 (2H)-yl)-1H-pyrrolo[2,3-b]pyridin-2-yl)ethyl)-1,2,3,6-tetrahydro-pyridin-4-yl)benzoic acid (4.8 g). Int. 2G117 (4.8 g) was resolved by SFC on a SEPIATEC-200-002 instrument fitted with a (R,R) Whelk-01 250×30 mm 5 μm column operated at 30° C. and an eluent of 70% CO₂ and 30% MeOH containing 0.5% MeONH₃ at a (100 g/min) and a back pressure of 120 bar to give Int. 2G118 (1.7 g, first peak) and Int. 2G119 (1.6 g, second peak) (S)-3,5-difluoro-4-(1-(1-(1-methyl-4-(4-((methylamino)-2-oxopyridin-1 (2H)-yl)-1H-pyrrolo[2,3-b]pyridin-2-yl)ethyl)-1,2,3,6-tetrahydropyridin-4-yl)benzoic acid and (R)-3,5-difluoro-4-(1-(1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoic acid. The absolute configurations of these compounds were not determined.

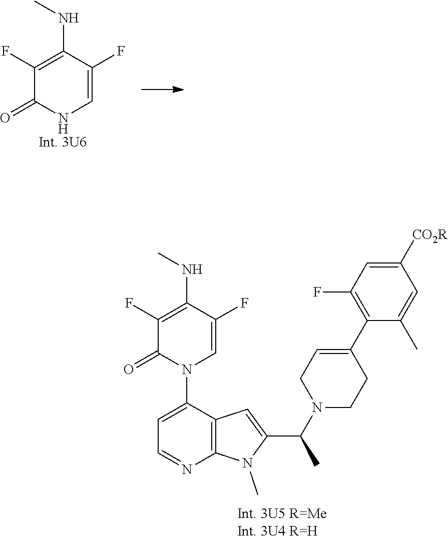

Paraformaldehyde (0.81 g) and 4-amino-3,5-difluoropyridin-2 (1H)-one (1.3 g) were stirred 6 h in MeOH (30 mL) and NaOMe (30% in MeOH, 4 mL) at 0° C. to 55° C. under an inert atmosphere. NaBH₄ (1.0 g) was added portion-wise and stirring was continued 4 h at 0° C. to 55° C. The residue after concentration was diluted with aq. citric acid to precipitate Int. 3U6 3,5-difluoro-4-(methylamino)-pyridin-2 (1H)-one (1 g). CuI (0.78 g), Ints. 3U6/1AF86 (0.43 g/l g), and K₃PO₄ (1.3 g) were stirred 16 h in dioxane (50 mL) and trans-N,N′-dimethylcyclohexane-1,2-diamine (0.58 mL) at 110° C. under an inert atmosphere. The mixture was filtered. The OL (aq. NH₃/EtOAc) was dried, filtered, concentrated, and purified by FC (DCM/MeOH 19:1 to 9:1) to give Int. 3U5 methyl (S)-4-(1-(1-(4-(3,5-difluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-3-fluoro-5-methyl-benzoate (0.20 g). LiOH·H₂O (45 mg) and Int. 3U5 (0.20 g) were stirred 16 h in THF/MeOH/H₂O (3:2:1, 12 mL) and concentrated. The AL (water/Et₂O) was acidified (pH adjusted to 5 using aq. Citric acid), filtered and dried to give Int. 3U4 (S)-4-(1-(1-(4-(3,5-difluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-3-fluoro-5-methylbenzoic acid (0.18 g).

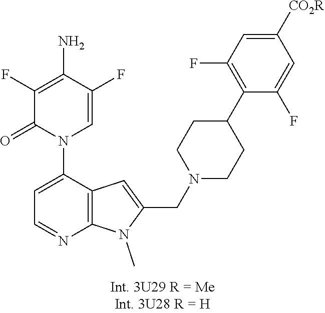

CuI (0.80 g), Int. 3C15 (1 g), 4-amino-3,5-difluoropyridin-2 (1H)-one (0.40 g), and K₃PO4 (1.3 g) were stirred 16 h in dioxane (10 mL) and trans-N,N′-dimethylcyclohexane-1,2-diamine (0.66 mL) at 110° C. under an inert atmosphere. The OL (water/EtOAc) was dried, filtered, concentrated, and purified by FC (PE/EtOAc 1:0 to 0:1) to give Int. 3U29 methyl 4-(1-((4-(4-amino-3,5-difluoro-2-oxopyridin-1 (2H)-yl)-1-methyl-1H-pyrrolo[2,3-b]pyridin-2-yl)methyl)piperidin-4-yl)-3,5-difluorobenzoate (0.60 g). LiOH·H₂O (0.21 g) and Int. 3U29 (0.55 g) were stirred 4 h in THF/MeOH/H₂O (2:1:1, 5.8 mL). The mixture was concentrated. The AL (water/Et₂O) was diluted with aq. citric acid to precipitate Int. 3U28 4-(1-((4-(4-amino-3,5-difluoro-2-oxopyridin-1 (2H)-yl)-1-methyl-1H-pyrrolo[2,3-b]pyridin-2-yl)methyl)piperidin-4-yl)-3,5-difluorobenzoic acid (0.35 g).

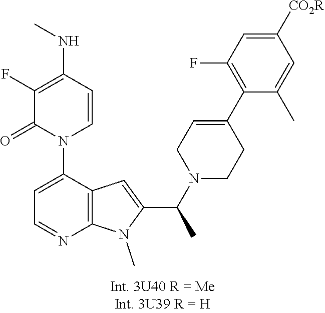

CuI (0.59 g), Ints. JJ44/1AF86 (0.66 g/1.5 g), and K₃PO₄ (2.0 g) were stirred 12 h in dioxane (40 mL) and trans-N,N′-dimethylcyclohexane-1,2-diamine (0.97 mL) at 110° C. under an inert atmosphere. The mixture was filtered. The OL (aq. NH₃/DCM/MeOH) was dried, filtered, concentrated, and triturated in Et₂O/EtOAc (4:1) to give Int. 3U48 methyl (S)-3-fluoro-4-(1-(1-(4-(3-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoate (1.4 g). LiOH·H₂O (0.52 g) and Int. 3U40 (1.4 g) were stirred 16 h in THF/MeOH/H₂O (4:1:2, 20 mL) at 0° C. to RT. The residue after concentration was stirred in aq. citric acid to precipitate Int. 3U39 (S)-3-fluoro-4-(1-(1-(4-(3-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoic acid (1.2 g).

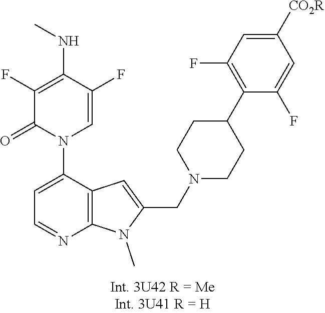

CuI (0.79 g), Ints. 3U6/3C15 (0.44 g/1.0 g), and K₃PO₄ were stirred 12 h dioxane (10 mL) and trans-N,N′-dimethylcyclohexane-1,2-diamine (0.65 mL) at 110° C. under an inert atmosphere. The mixture was filtered. The OL (water/EtOAc) was dried, filtered, concentrated, and purified by FC (DCM/MeOH 1:0 to 9:1) to give Int. 3U42 methyl 4-(1-((4-(3,5-difluoro-4-(methylamino)-2-oxopyridin-1 (2H)-yl)-1-methyl-1H-pyrrolo[2,3-b]pyridin-2-yl)methyl)piperidin-4-yl)-3,5-difluorobenzoate (0.35 g). LiOH·H₂O (0.13 g) and Int. 3U42 (0.35 g) were stirred 2 h in THF/MeOH/H₂O (2:1:2, 12.5 mL) at 0-60° C. The residue after concentration was stirred in aq. HCl to precipitate Int. 3U41 4-(1-((4-(3,5-difluoro-4-(methylamino)-2-oxopyridin-1 (2H)-yl)-1-methyl-1H-pyrrolo[2,3-b]pyridin-2-yl)methyl)-piperidin-4-yl)-3,5-difluorobenzoic acid (0.25 g).

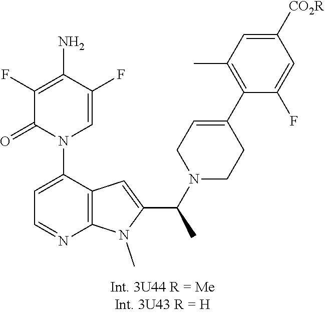

CuI (0.78 g), Int. 1AF86 (1 g), 4-amino-3,5-difluoropyridin-2 (1H)-one (0.39 g), and K₃PO4 (1.3 g) were stirred 16 h in dioxane (15 mL) and trans-N,N′-dimethylcyclohexane-1,2-diamine (0.58 mL) at 150° C. under an inert atmosphere. The mixture was filtered. The OL (aq. NH₃/DCM/MeOH) was dried, filtered, concentrated, and purified by FC (DCM/MeOH 19:1 to 9:1) to give Int. 3U44 methyl (S)-4-(1-(1-(4-(4-amino-3,5-difluoro-2-oxopyridin-1 (2H)-yl)-1-methyl-1H-pyrrolo[2,3-b]pyridin-2-yl)ethyl)-1,2,3,6-tetrahydropyridin-4-yl)-3-fluoro-5-methylbenzoate (0.48 g). LiOH·H₂O (0.15 g) and Int. 3U44 (0.40 g) were stirred 4 h in THF/MeOH/H₂O (3:2:1, 24 mL). The mixture was concentrated. The AL (water/Et₂O) was diluted with aq. citric acid to precipitate Int. 3U43 (S)-4-(1-(1-(4-(4-amino-3,5-difluoro-2-oxopyridin-1 (2H)-yl)-1-methyl-1H-pyrrolo[2,3-b]pyridin-2-yl)ethyl)-1,2,3,6-tetrahydro-pyridin-4-yl)-3-fluoro-5-methylbenzoic acid (0.38 g).

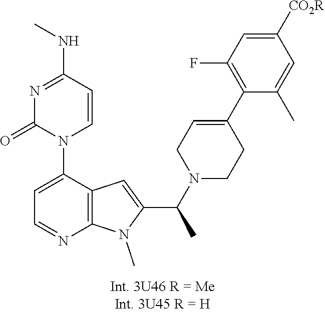

CuI (0.78 g), Int. HH2 (1 g), 4-(methylamino)pyrimidin-2 (1H)-one (0.33 g), and K₃PO₄ (1.3 g) were stirred 16 h in DMF/DMSO (10:3, 13 mL) and trans-N,N′-dimethylcyclohexane-1,2-diamine (0.65 mL) at 110° C. under an inert atmosphere. The OL (water/EtOAc) was dried, filtered, and concentrated to give Int. 3U46 methyl (S)-3-fluoro-5-methyl-4-(1-(1-(1-methyl-4-(4-(methylamino)-2-oxopyrimidin-1 (2H)-yl)-1H-pyrrolo[2,3-b]pyridin-2-yl)ethyl)-1,2,3,6-tetrahydropyridin-4-yl)benzoate (0.80 g). LiOH·H₂O (0.19 g) and Int. 3U46 (0.80 g) were stirred 4 h in THF/MeOH/H₂O (2:1:1, 21 mL). The mixture was concentrated. The AL (water/Et₂O) was diluted with aq. citric acid to precipitate Int. 3U45 (S)-3-fluoro-5-methyl-4-(1-(1-(1-methyl-4-(4-(methylamino)-2-oxopyrimidin-1 (2H)-yl)-1H-pyrrolo[2,3-b]pyridin-2-yl)ethyl)-1,2,3,6-tetrahydropyridin-4-yl)benzoic acid (0.75 g).

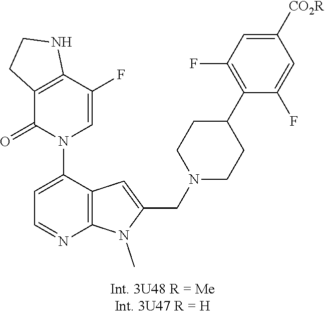

CuI (0.10 g), Ints. VV14/3C15 (0.24 g/0.50 g), and Cs₂CO₃ (1.0 g) were stirred 16 h in dioxane (5 mL) and trans-N,N′-dimethylcyclohexane-1,2-diamine (0.16 mL) at 90° C. under an inert atmosphere. The mixture was filtered. The OL (aq. NH₃/DCM/MeOH) was dried, filtered, concentrated, and purified by FC (DCM/MeOH 19:1) to give Int. 3U48 methyl 3,5-difluoro-4-(1-((4-(7-fluoro-4-oxo-1,2,3,4-tetrahydro-5H-pyrrolo-[3,2-c]pyridin-5-yl)-1-methyl-1H-pyrrolo[2,3-b]pyridin-2-yl)methyl)piperidin-4-yl)benzoate (0.35 g). LiOH·H₂O (0.11 g) and Int. 3U48 (0.30 g) were stirred 16 h in THF/MeOH/H₂O (2:2:1, 6 mL) at 0° C. to RT. The residue after concentration was stirred in aq. citric acid to precipitate Int. 3U47 3,5-difluoro-4-(1-((4-(7-fluoro-4-oxo-1,2,3,4-tetrahydro-5H-pyrrolo[3,2-c]pyridin-5-yl)-1-methyl-1H-pyrrolo[2,3-b]pyridin-2-yl)methyl)piperidin-4-yl)benzoic acid (0.25 g).

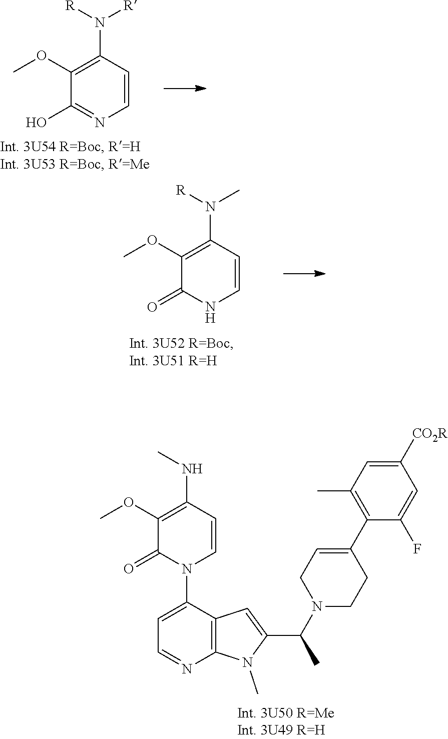

2-Chloro-4-iodo-3-methoxypyridine (3 g), tert-butyl carbamate (2.6 g), Pd₂(dba)₃ (0.31 g), XantPhos (0.32 g), and Cs₂CO₃ (11 g) were stirred 16 h in toluene (10 mL) at 110° C. under an inert atmosphere. The mixture was filtered. The OL (water/EtOAc) was dried, filtered, concentrated, combined with a smaller batch and purified by FC (hexane/EtOAc 2:1) to give Int. 3U54 tert-butyl (2-chloro-3-methoxy-pyridin-4-yl)carbamate (3.5 g). Int. 3U54 (3.5 g) was added to a solution of NaH (0.65 g) in DMF (50 mL) and stirred for 0.5 h at RT. MeI (1.7 mL) was added at 0° C. and stirring continued for 1 h. The OL (water/EtOAc) was dried, filtered, concentrated, and purified by FC (hexane/EtOAc 1:4) to give Int. 3U53 tert-butyl (2-chloro-3-methoxypyridin-4-yl)(methyl)carbamate (3.1 g). Int. 3U53 (1.5 g), Pd₂(dba)₃ (0.25 g), and 5-(di-tert-butylphosphino)-1′, 3′, 5‘-triphenyl-1’H-[1,4′]bipyrazole (0.28 g) were stirred 16 h in dioxane (25 mL) and 50% aq. CsOH (5 mL) at 110° C. under an inert atmosphere. The AL (water/EtOAc) was diluted with aq. citric acid and sat. aq. NaCl and extracted with DCM/MeOH. The OL was dried, filtered, and concentrated to give Int. 3U52 tert-butyl (3-methoxy-2-oxo-1,2-dihydropyridin-4-yl)(methyl)carbamate (1.0 g). Int. 3U52 (1 g) was stirred 16 h in DCM/4M HCl in dioxane (2:1, 15 mL) at 0° C. to RT under an inert atmosphere. The mixture was concentrated and washed with pentane to give Int. 3U51 3-methoxy-4-(methylamino)-pyridin-2 (1H)-one (0.6. g, HCl salt). CuI (0.32 g), Ints. 3U51/1AF86 (0.33 g/0.80 g), and K₃PO₄ (1.0 g) were stirred 16 h in dioxane (15 mL) and trans-1,2-diaminocyclohexane (0.42 mL) at 110° C. under an inert atmosphere. The mixture was filtered. The OL (aq. NH₃/DCM/MeOH) was dried, filtered, concentrated, and triturated with DCM/MeOH (9:1) to give Int. 3U50 methyl (S)-3-fluoro-4-(1-(1-(4-(3-methoxy-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoate (0.56 g). LiOH·H₂O (0.15 g) and Int. 3U50 (0.40 g) were stirred 16 h in THF/H₂O (3:2, 25 mL). The residue after concentration was stirred in aq. citric acid to precipitate Int. 3U49 (S)-3-fluoro-4-(1-(1-(4-(3-methoxy-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoic acid (0.35 g).

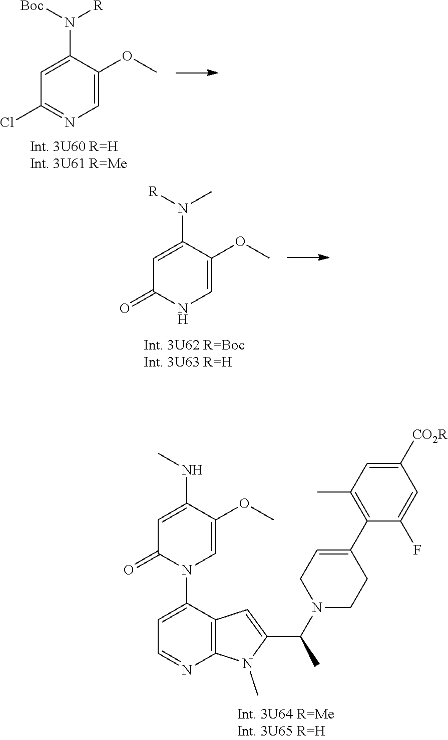

Pd₂(dba)₃ (4.3 g), XantPhos (4.5 g), 2-chloro-4-iodo-5-methoxypyridine (42 g), tert-butyl carbamate (27 g), and Cs₂CO₃ (152 g) were stirred 16 h in toluene (500 mL) at 110° C. under an inert atmosphere. The OL (water/EtOAc) was dried, filtered, concentrated, and purified by FC (hexane/EtOAc 4:1 to 2:1) to give Int. 3U60 tert-butyl (2-chloro-5-methoxypyridin-4-yl)carbamate (30 g). NaH (1.7 g) and Int. 3U60 (10 g) were stirred 0.5 h in DMF (100 mL) at 0° C. to RT. MeI (4.8 mL) was added and stirring continued 4 h at 0° C. to RT. The mixture diluted with water to precipitate a solid that was triturated in pentane/EtOAc (1:1) to give Int. 3U61 tert-butyl (2-chloro-5-methoxypyridin-4-yl)(methyl)-carbamate (9 g). Pd₂(dba)₃ (0.34 g), 5-(di-tert-butylphosphino)-1′, 3′, 5′-triphenyl-1′H-[1,4′]bipyrazole (0.37 g), and Int. 3U61 (2 g) were stirred 16 h in dioxane (25 mL) and CsOH (50% in aq., 20 mL) at 115° C. under an inert atmosphere. The AL (water/DCM) was adjusted to pH 4-5 with aq. citric acid. The OL (water pH 4-5/DCM/MeOH) was dried, filtered, and concentrated to give Int. 3U62 tert-butyl (5-methoxy-2-oxo-1,2-dihydropyridin-4-yl)(methyl)-carbamate (1 g). Int. 3U62 (0.55 g) was stirred 16 h in DCM/4M HCl under an inert atmosphere. The mixture was concentrated and washed with pentane and Et₂O to give Int. 3U63 5-methoxy-4-(methylamino)pyridin-2 (1H)-one (0.33 g, HCl salt). CuI (0.78 g), Ints. 3U63/1AF86 (0.41 g/1.0 g), and K₃PO₄ (1.3 g) were stirred 20 h in dioxane (20 mL) and trans-N,N′-dimethylcyclohexane-1,2-diamine (0.6 mL) at 80° C. under an inert atmosphere. The mixture was filtered. The OL (aq. NH₃/DCM/MeOH) was dried, concentrated, and washed with pentane and Et₂O to give Int. 3U64 methyl (S)-3-fluoro-4-(1-(1-(4-(5-methoxy-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoate (1.0 g). LiOH·H₂O (0.17 g) and Int. 3U64 (0.57 g) in were stirred 16 h in THF/MeOH/H₂O (10:8:5, 23 mL). The residue after concentration was stirred in aq. citric acid to precipitate Int. 3U65 (S)-3-fluoro-4-(1-(1-(4-(5-methoxy-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoic acid (0.50 g).

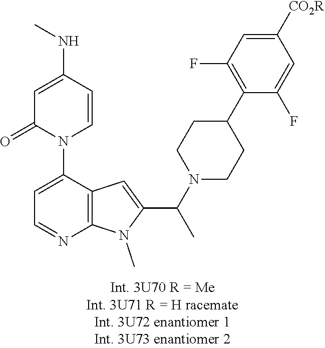

CuI (3.0 g), Ints. 2G81/2G96 (1.3 g/4.0 g), and K₃PO₄ (5.0 g) were stirred 16 h in dioxane (100 mL) and trans-N,N′-dimethylcyclohexane-1,2-diamine (2 mL) at 100° C. under an inert atmosphere. The mixture was filtered. The OL (aq. NH₃/DCM/MeOH) was dried, concentrated, and washed with Et₂O to give Int. 3U70 methyl 3,5-difluoro-4-(1-(1-(1-methyl-4-(4-(methylamino)-2-oxopyridin-1 (2H)-yl)-1H-pyrrolo[2,3-b]pyridin-2-yl)ethyl)piperidin-4-yl)benzoate (3.8 g). LiOH·H₂O (1.1 g) and Int. 3U70 (4 g) were stirred 16 h in THF/MeOH/H₂O (2:3:1, 20 mL) at 0° C. to RT. The mixture was concentrated. The AL (water/Et₂O) was diluted with aq. citric acid to precipitate Int. 3U71 3,5-difluoro-4-(1-(1-(1-methyl-4-(4-(methylamino)-2-oxopyridin-1 (2H)-yl)-1H-pyrrolo[2,3-b]pyridin-2-yl)ethyl)piperidin-4-yl)benzoic acid (3.8 g). Int. 3U71 (3.8 g) was resolved by SFC on a SEPIATEC-250 instrument fitted with a (R,R) WHELK PACKED 250×30 mm 5 μm column operated at 30° C. and an eluent of 70% CO₂ and 30% MeOH containing 0.5% MeONH₃ at a (100 g/min) and a back pressure of 100 bar to give Int. 3U72 (1.3 g, first peak) and Int. 3U73 (1.3 g, second peak) (S)-3,5-difluoro-4-(1-(1-(1-methyl-4-(4-(methylamino)-2-oxopyridin-1 (2H)-yl)-1H-pyrrolo[2,3-b]pyridin-2-yl)ethyl)piperidin-4-yl)benzoic acid and (R)-3,5-difluoro-4-(1-(1-(1-methyl-4-(4-(methylamino)-2-oxopyridin-1 (2H)-yl)-1H-pyrrolo[2,3-b]pyridin-2-yl)ethyl)piperidin-4-yl)benzoic acid. The absolute configurations of these compounds were not determined.

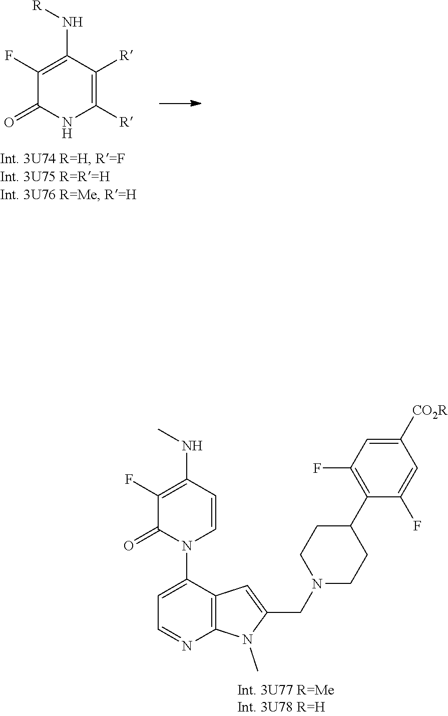

2,3,5,6-tetrafluoropyridin-4-amine (30 g) was stirred 16 h in 1M aq. NaOH (400 mL) 100° C. pH was adjusted to 4 with conc. aq. HCl. The OL (water/EtOAc) was dried, filtered, concentrated, and triturated in Et₂O to give Int. 3U74 4-amino-3,5,6-trifluoropyridin-2 (1H)-one (24 g). Int. 3U74 (5.0 g) was stirred 16 h in EtOH (120 mL) and N₂H₄H₂O (20 mL) at 100° C. The mixture was concentrated, purified by FC (DCM/MeOH 3:1), and triturated in DCM/DMSO (19:1) to give Int. 3U75 4-amino-3-fluoropyridin-2 (1H)-one (1.5 g). Paraformaldehyde (3.5 g) and Int. 3U75 (1.5 g) were stirred 4 h in MeOH (20 mL) and NaOMe (30% in MeOH, 10 mL) at 0° C. to 55° C. NaBH₄ (1.4 g) was added and stirring continued 4 h at 0° C. to 55° C. The mixture diluted with aq. citric acid, concentrated, washed with DCM/MeOH (3:2), dried, and purified by FC on neutral alumina (DCM/MeOH 17:3) to give Int. 3U76 3-fluoro-4-(methylamino)pyridin-2 (1H)-one (0.60 g). Int. 3U77 methyl 3,5-difluoro-4-(1-((4-(3-fluoro-4-(methylamino)-2-oxopyridin-1 (2H)-yl)-1-methyl-1H-pyrrolo[2,3-b]pyridin-2-yl)methyl)-piperidin-4-yl)benzoate (1.2 g) was prepared similarly to Int. 3U70 from Ints. 3U76/3C15 (0.58 g/1.5 g). LiOH·H₂O (0.46 g) and Int. 1122 (1.2 g) were stirred 16 h in THF/MeOH/H₂O (6:2:3, 22 mL) at 0° C. to RT. The residue after concentration was stirred in aq. citric acid to precipitate Int. 3U78 3,5-difluoro-4-(1-((4-(3-fluoro-4-(methylamino)-2-oxopyridin-1 (2H)-yl)-1-methyl-1H-pyrrolo[2,3-b]pyridin-2-yl)methyl)piperidin-4-yl)benzoic acid (1.0 g).

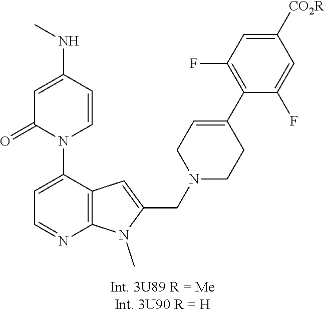

CuI (4.8 g), Ints. 2G81/3C31 (2.0 g/6.0 g), and K₃PO₄ (8.0 g) were stirred 3 h in dioxane (70 mL) and trans-N,N′-dimethylcyclohexane-1,2-diamine (4 mL) at 100° C. under an inert atmosphere. The mixture was filtered. The OL (water/EtOAc) was dried, concentrated, and purified by FC (PE/EtOAc 1:4) to give Int. 3U89 methyl 3,5-difluoro-4-(1-((1-methyl-4-(4-(methylamino)-2-oxopyridin-1 (2H)-yl)-1H-pyrrolo[2,3-b]pyridin-2-yl)methyl)-1,2,3,6-tetrahydropyridin-4-yl)benzoate (3.1 g). LiOH·H₂O (1.0 g) and Int. 3U89 (2.5 g) were stirred 3 h in THF/MeOH/H₂O (5:1:1, 35 mL) at 0° C. to RT. The residue after concentration was stirred in aq. HCl to precipitate Int. 3U90 3,5-difluoro-4-(1-((1-methyl-4-(4-(methylamino)-2-oxopyridin-1 (2H)-yl)-1H-pyrrolo[2,3-b]pyridin-2-yl)methyl)-1,2,3,6-tetrahydropyridin-4-yl)benzoic acid (2.0 g).

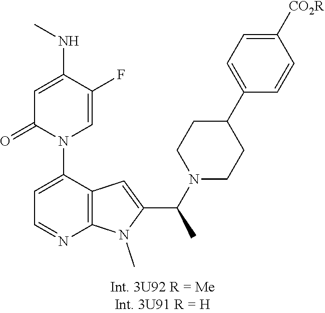

CuI (5.0 g), Ints. 2G68/3E35 (2.4 g/6.0 g), and K₃PO₄ (8.4 g) were stirred 16 h in dioxane (30 mL) and trans-N,N′-dimethylcyclohexane-1,2-diamine (4 mL) at 110° C. under an inert atmosphere. The mixture was filtered. The OL (aq. NH₃/DCM/MeOH) was dried, concentrated, and purified by FC (DCM/MeOH 9:1) to give Int. 3U92 methyl (S)-4-(1-(1-(4-(5-fluoro-4-(methylamino)-2-oxopyridin-1 (2H)-yl)-1-methyl-1H-pyrrolo[2,3-b]pyridin-2-yl)ethyl)piperidin-4-yl)benzoate (4.0 g). LiOH (0.97 g) and Int. 3U92 (4.0 g) were stirred 16 h in THF/MeOH/H₂O (3:2:1, 35 mL) at 0° C. to RT. The residue after concentration was stirred in aq. citric acid to precipitate Int. 3U91 (S)-4-(1-(1-(4-(5-fluoro-4-(methylamino)-2-oxopyridin-1 (2H)-yl)-1-methyl-1H-pyrrolo[2,3-b]pyridin-2-yl)ethyl)piperidin-4-yl)benzoic acid (4.8 g).

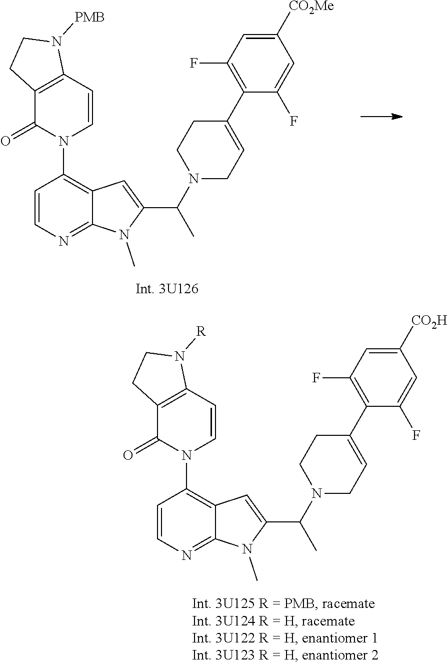

Int. 3U126 methyl 3,5-difluoro-4-(1-(1-(4-(1-(4-methoxybenzyl)-4-oxo-1,2,3,4-tetrahydro-5H-pyrrolo[3,2-c]pyridin-5-yl)-1-methyl-1H-pyrrolo[2,3-b]pyridin-2-yl)ethyl)-1,2,3,6-tetrahydropyridin-4-yl)benzoate (2.1 g) was prepared similarly to Int. 3U89 from Ints. 3C26/3E181 (3.3 g/1.9 g) and purified by FC (DCM/MeOH 1:0 to 9:1). LiOH·H₂O (0.66 g) and Int. 3U126 (2.1 g) were stirred 6 h in THF/MeOH/H₂O (2:2:1, 24 mL) at 0-60° C. The residue after concentration was stirred in aq. HCl to precipitate Int. 3U125 3,5-difluoro-4-(1-(1-(4-(1-(4-methoxybenzyl)-4-oxo-1,2,3,4-tetrahydro-5H-pyrrolo[3,2-c]pyridin-5-yl)-1-methyl-1H-pyrrolo[2,3-b]pyridin-2-yl)ethyl)-1,2,3,6-tetrahydropyridin-4-yl)benzoic acid (1.7 g). Int. 3U125 (1.7 g) was stirred 12 h in TFA (6 mL) at 65° C. under an inert atmosphere. The residue after concentration washed with Et₂O to give Int. 3U124 3,5-difluoro-4-(1-(1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzoic acid (1.4 g, TFA salt). Int. 3U124 (1.7 g) was resolved by SFC on a SFC-150-008 instrument fitted with a (R,R) Whelk-01 250×30 mm 5 μm column operated at 30° C. and an eluent of 60% CO₂ and 40% MeOH containing 0.5% MeONH₃ at a (100 g/min) and a back pressure of 120 bar to give Int. 3U122 (0.20 g, first peak) and Int. 3U123 (0.16 g, second peak) (R)-3,5-difluoro-4-(1-(1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzoic acid and (S)-3,5-difluoro-4-(1-(1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzoic acid. The absolute configurations of these compounds were not determined.

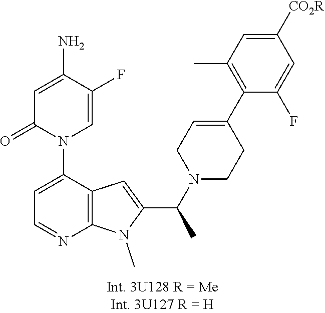

CuI (78 mg), 4-amino-5-fluoropyridin-2 (1H)-one (53 mg), Int. 1AF86 (0.10 g), and K₃PO4 (0.13 g) were stirred 16 h in dioxane (5 mL) and trans-N,N′-Dimethylcyclohexane-1,2-diamine (65 μL) at 100° C. under an inert atmosphere. The mixture was combined with two other batches prepared similarly and filtered. The OL (water/EtOAc) was dried, concentrated, and purified by FC (hexane/EtOAc 1:1 to DCM/MeOH 9:1) to give Int. 3U128 methyl (S)-4-(1-(1-(4-(4-amino-5-fluoro-2-oxopyridin-1 (2H)-yl)-1-methyl-1H-pyrrolo[2,3-b]pyridin-2-yl)ethyl)-1,2,3,6-tetrahydropyridin-4-yl)-3-fluoro-5-methyl-benzoate (0.16 g). LiOH·H₂O (63 mg) and Int. 3U128 (0.16 g) were stirred 2 h in THF/MeOH/H₂O (1:1:1, 3 mL) at 0° C. to RT. The residue after concentration was stirred in aq. citric acid to precipitate Int. 3U127 (S)-4-(1-(1-(4-(4-amino-5-fluoro-2-oxopyridin-1 (2H)-yl)-1-methyl-1H-pyrrolo[2,3-b]-pyridin-2-yl)ethyl)-1,2,3,6-tetrahydropyridin-4-yl)-3-fluoro-5-methylbenzoic acid (0.11 g).

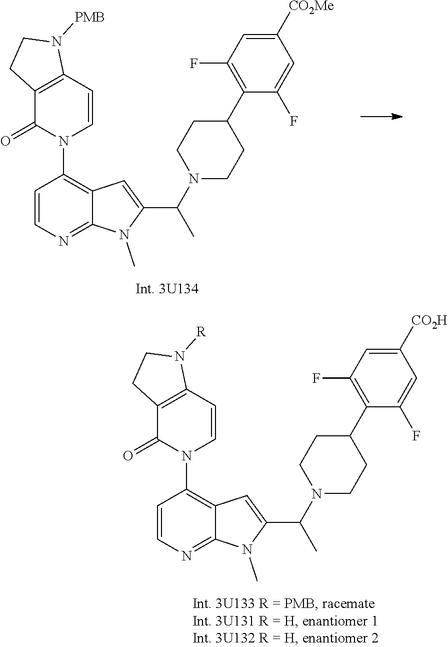

CuI (1.1 g), Ints. 3E181/2G96 (0.87 g/1.4 g), and K₃PO₄ (1.8 g) were stirred 16 h in dioxane (8 mL) and trans-N,N′-dimethylcyclohexane-1,2-diamine (0.9 mL) at 120° C. under an inert atmosphere. The mixture was filtered. The OL (water/EtOAc) was dried, concentrated, and purified by FC (DCM/MeOH 19:1 to 9:1) to give Int. 3U134 methyl 3,5-difluoro-4-(1-(1-(4-(1-(4-methoxybenzyl)-4-oxo-1,2,3,4-tetrahydro-5H-pyrrolo[3,2-c]pyridin-5-yl)-1-methyl-1H-pyrrolo[2,3-b]pyridin-2-yl)ethyl)piperidin-4-yl)benzoate (1.1 g). LiOH·H₂O (0.35 g) and Int. 3U134 (1.1 g) were stirred 16 h in THF/MeOH/H₂O (2:2:1, 20 mL). The residue after concentration was stirred in aq. citric acid to precipitate Int. 3U133 3,5-difluoro-4-(1-(1-(4-(1-(4-methoxybenzyl)-4-oxo-1,2,3,4-tetrahydro-5H-pyrrolo[3,2-c]pyridin-5-yl)-1-methyl-1H-pyrrolo[2,3-b]pyridin-2-yl)ethyl)piperidin-4-yl)benzoic acid (0.77 g). Int. 3U133 (0.98 g) was stirred 2 h in TFA (6 mL) and resolved by SFC on a (R,R) SEPIATEC-200-001 instrument fitted with a (R,R) Whelk-01 250×30 mm 5 μm column operated at 30° C. and an eluent of 65% CO₂ and 35% 30 mM MeONH₃ in MeOH at a (90 g/min) and a back pressure of 130 bar to give Int. 3U131 (0.12 g, first peak) and Int. 3U132 (0.13 g, second peak) (S)-3,5-difluoro-4-(1-(1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)piperidin-4-yl)benzoic acid and (R)-3,5-difluoro-4-(1-(1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)piperidin-4-yl)benzoic acid. The absolute configurations of these compounds were not determined.

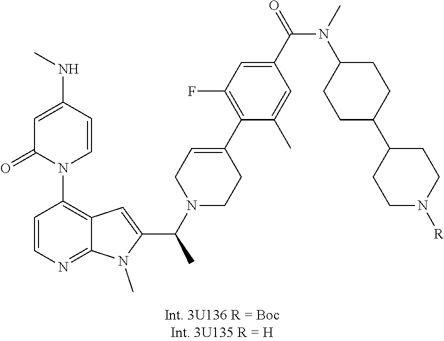

HATU (0.73 g), tert-butyl 4-[4-(methylamino)cyclohexyl]piperidine-1-carboxylate (0.38 g), and Int. 2G46 (0.73 g) were stirred ON in DIPEA (0.4 mL) and DMF (4 mL). The OL (EtOAc/brine) was dried and concentrated to give Int. 3u136 tert-butyl 4-[4-[[3-fluoro-5-methyl-4-[1-[(1S)-1-[1-methyl-4-[4-(methylamino)-2-oxo-1-pyridyl]pyrrolo[2,3-b]pyridin-2-yl]ethyl]-3,6-dihydro-2H-pyridin-4-yl]-benzoyl]-methyl-amino]cyclohexyl]piperidine-1-carboxylate (0.70 g). This material was stirred 1 h in HFIP (10 mL) and 37% aq. HCl (1 mL). The residue after concentration was HPLC-purified to give Int. 3U135 3-fluoro-N,5-dimethyl-4-[1-[(1S)-1-[1-methyl-4-[4-(methylamino)-2-oxo-1-pyridyl]-pyrrolo-[2,3-b]pyridin-2-yl]ethyl]-3,6-dihydro-2H-pyridin-4-yl]-N-[4-(4-piperidyl)cyclohexyl]benzamide (0.15 g, HCl salt).

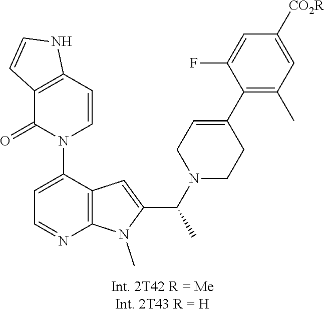

Int. 2T42 methyl (R)-3-fluoro-5-methyl-4-(1-(1-(1-methyl-4-(4-oxo-1,4-dihydro-5H-pyrrolo[3,2-c]pyridin-5-yl)-1H-pyrrolo[2,3-b]pyridin-2-yl)ethyl)-1,2,3,6-tetrahydropyridin-4-yl)benzoate (1.4 g) was prepared similarly to Int. 2G21 from 1,5-dihydro-4H-pyrrolo[3,2-c]pyridin-4-one (1.7 g) and Int. 1AF86′ (2 g). Int. 2T43 (R)-3-fluoro-5-methyl-4-(1-(1-(1-methyl-4-(4-oxo-1,4-dihydro-5H-pyrrolo[3,2-c]pyridin-5-yl)-1H-pyrrolo[2,3-b]pyridin-2-yl)ethyl)-1,2,3,6-tetrahydropyridin-4-yl)benzoic acid (20 mg) was prepared similarly to Int. 2G41 from Int. 2T42 (50 g).

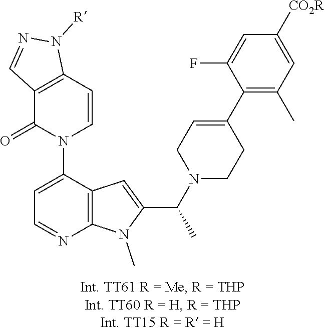

Int. 2T44 methyl 3-fluoro-5-methyl-4-(1-((1R)-1-(1-methyl-4-(4-oxo-1-(tetrahydro-2H-pyran-2-yl)-1,4-dihydro-5H-pyrazolo[4,3-c]pyridin-5-yl)-1H-pyrrolo[2,3-b]pyridin-2-yl)ethyl)-1,2,3,6-tetrahydro-pyridin-4-yl)benzoate (1 g) was prepared similarly to Int. 2G21 from Ints. 1Z82/1AF86′ (0.97 g/1 g). Int. 2T45 3-fluoro-5-methyl-4-(1-((1R)-1-(1-methyl-4-(4-oxo-1-(tetrahydro-2H-pyran-2-yl)-1,4-dihydro-5H-pyrazolo[4,3-c]pyridin-5-yl)-1H-pyrrolo[2,3-b]pyridin-2-yl)ethyl)-1,2,3,6-tetrahydropyridin-4-yl)benzoic acid (0.75 g) was prepared similarly to Int. VV3 from Int. 2T44 (0.80 g). Int. 2T45 (0.70 g) was stirred in DCM/TFA (1:1, 20 mL) for 16 h. The mixture was concentrated, triturated in Et₂O to give Int. 2T46 (R)-3-fluoro-5-methyl-4-(1-(1-(1-methyl-4-(4-oxo-1,4-dihydro-5H-pyrazolo[4,3-c]pyridin-5-yl)-1H-pyrrolo[2,3-b]pyridin-2-yl)ethyl)-1,2,3,6-tetrahydropyridin-4-yl)benzoic acid (0.61 g).

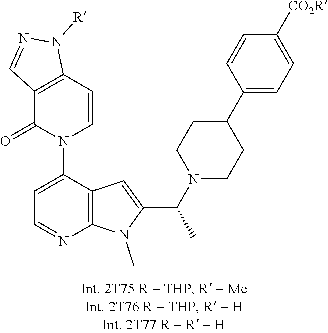

Int. 2T75 methyl 4-(1-((1R)-1-(1-methyl-4-(4-oxo-1-(tetrahydro-2H-pyran-2-yl)-1,4-dihydro-5H-pyrazolo[4,3-c]pyridin-5-yl)-1H-pyrrolo[2,3-b]pyridin-2-yl)ethyl)piperidin-4-yl)benzoate (0.90 g) was prepared similarly to Int. 3U5 from Ints. 1Z82/3E36 (0.59 g/1.1 g). Int. 2T76 4-(1-((1R)-1-(1-methyl-4-(4-oxo-1-(tetrahydro-2H-pyran-2-yl)-1,4-dihydro-5H-pyrazolo[4,3-c]pyridin-5-yl)-1H-pyrrolo[2,3-b]-pyridin-2-yl)ethyl)piperidin-4-yl)benzoic acid was prepared similarly to Int. 3U4 from Int. 2T75 (0.40 g). Int. 2T76 (50 mg) was stirred in DCM/TFA (1:1, 1 mL) for 16 h. The mixture was concentrated, triturated in Et₂O to give Int. 2T77 (R)-4-(1-(1-(1-methyl-4-(4-oxo-1,4-dihydro-5H-pyrazolo[4,3-c]-pyridin-5-yl)-1H-pyrrolo[2,3-b]pyridin-2-yl)ethyl)piperidin-4-yl)benzoic acid (40 mg, TFA salt).

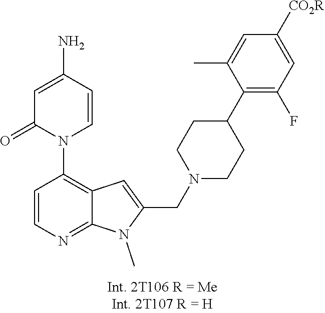

Int. 2G115 (0.50 g), 4-amino-1H-pyridin-2-one (0.15 g), CuI (0.02 g), trans-N,N′-dimethylcyclohexane-1,2-diamine (0.30 g), and K₃PO₄ (0.89 g) were stirred 2 h in dioxane (10 mL) at 130° C. under MW conditions. The mixture was filtered. The OL (EtOAc/aq. NH₃) was dried and concentrated to give Int. 2T106 methyl 4-[1-[[4-(4-amino-2-oxo-1-pyridyl)-1-methyl-pyrrolo[2,3-b]pyridin-2-yl]methyl]-4-piperidyl]-3-fluoro-5-methyl-benzoate (0.45 g). Int. 2T106 (0.90 g) and LiOH—H₂O (0.37 g) were stirred 12 h in THF/MeOH/water (2:2:1; 15 mL) at 0° C. to RT. The residue after concentration was stirred in aq. citric acid to precipitate Int. 2T107 4-[1-[[4-(4-amino-2-oxo-1-pyridyl)-1-methyl-pyrrolo[2,3-b]-pyridin-2-yl]methyl]-4-piperidyl]-3-fluoro-5-methyl-benzoic acid (0.78 g).

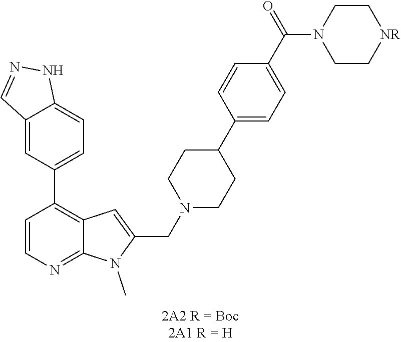

Int. 3A2 (0.20 g, HCl salt), tert-butyl piperazine-1-carboxylate (64 mg), and HATU (0.16 g) were stirred 2 h in DMF/DIPEA (5.7:1, 4 mL). The OL (EtOAc/water) was washed with brine, dried, concentrated, and HPLC-purified to give Int. 2A2 tert-butyl 4-[4-[1-[[1-methyl-4-(1-tetrahydro-pyran-2-ylindazol-5-yl)pyrrolo[2,3-b]-pyridin-2-yl]methyl]-4-piperidyl]benzoyl]-piperazine-1-carboxylate (0.21 g). This material was stirred 1 h in DCM/TFA (2:1, 6 mL). The mixture purified by SCX to give Int. 2A1 (4-(1-((4-(1H-indazol-5-yl)-1-methyl-1H-pyrrolo[2,3-b]pyridin-2-yl)methyl)piperidin-4-yl)phenyl)(piperazin-1-yl)methanone (0.15 g).

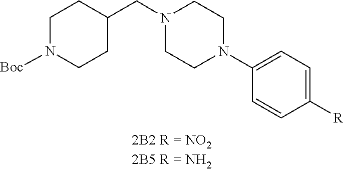

Int. 2B2 tert-butyl 4-((4-(4-nitrophenyl)piperazin-1-yl)methyl)piperidine-1-carboxylate (0.45 g) was prepared from 1-fluoro-4-nitrobenzene similarly to Int. 2I4.

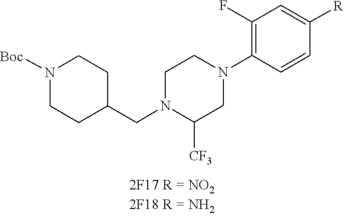

Int. 3E139 (0.36 g), 1,2-difluoro-4-nitrobenzene (162 mg), and K₂CO₃ (0.42 g) were stirred 48 h at 80° C. in ACN (5 mL). The OL (EtOAc/water) was concentrated and purified by FC (hexane/EtOAc 7:3) to give Int. 2F17 tert-butyl 4-((4-(2-fluoro-4-nitrophenyl)-2-(trifluoro-methyl)piperazin-1-yl)methyl)piperidine-1-carboxylate (0.30 g). Int. 2F17 (0.1 g) was hydrogenated ON using 10% Pd/C (20 mg) in THF/dioxane (3:1, 8 mL), filtered, and concentrated to give Int. 2F18 tert-butyl 4-((4-(4-amino-2-fluorophenyl)-2-(trifluoromethyl)piperazin-1-yl)methyl)-piperidine-1-carboxylate (50-mg).

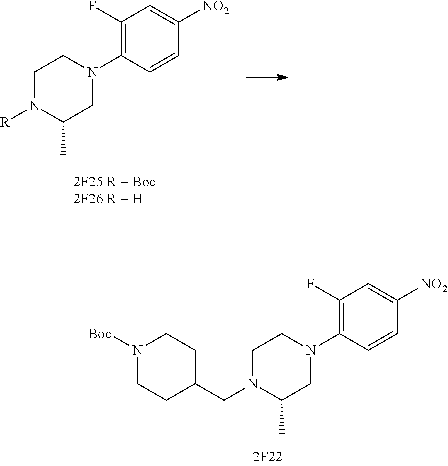

K₂CO₃ (1.36 g), 1,2-difluoro-4-nitro-benzene (0.52 g), and tert-butyl (S)-2-methyl-piperazine-1-carboxylate (0.65 g) were stirred ON in DMF (3 mL) at 70° C. The OL (EtOAc/water) was washed with 3M aq. CaCl₂ and brine, concentrated, and purified by FC (heptane/EtOAc 1:0 to 3:1) to give Int. 2F25 tert-butyl (S)-4-(2-fluoro-4-nitro-phenyl)-2-methylpiperazine-1-carboxylate (0.95 g). This material was stirred 1.5 h in 2.7M HCl in dioxane (15 mL) and concentrated to give Int. 2F26 (S)-1-(2-fluoro-4-nitro-phenyl)-3-methylpiperazine (0.77 g, HCl salt). Int. 2F26 (0.68 g) tert-butyl 4-formyl-piperidine-1-carboxylate (0.96 g) were stirred 0.25 h in DMF/DIPEA (5.3:1, 12 mL). STAB (2.4 g) was added and stirring continued ON. The OL (EtOAc/water) was washed with 3M aq. CaCl₂ and brine, concentrated, and purified by FC (heptane/EtOAc 1:0 to 3:2) to give Int. 2F22 tert-butyl (S)-4-((4-(2-fluoro-4-nitrophenyl)-2-methylpiperazin-1-yl)methyl)-piperidine-1-carboxylate (0.74 g).

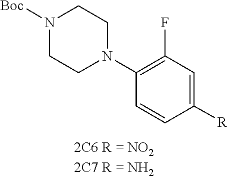

1,2-Difluoro-4-nitrobenzene (0.50 g), tert-butyl piperazine-1-carboxylate (0.70 g), and K₂CO₃ (0.65 g) were stirred ON in DMF (5 mL) at 80° C. The OL (water/EtOAc) was dried, concentrated, and purified by FC (EtOAc/pentane 0:1 to 1:4) to give Int. 2C6 tert-butyl 4-(2-fluoro-4-nitro-phenyl)piperazine-1-carboxylate (0.53 mg). Int. 2C6 (0.5 g), iron powder (0.43 g), and NH₄Cl (0.41 g) were stirred 2 h at 80° C. in THF/EtOH/water (4:4:3, 11 mL). The filtrate after filtration was concentrated and purified by FC (hexane/EtOAc 4:1 to 1:1) to give Int. 2C7 tert-butyl 4-(4-amino-2-fluorophenyl)-piperazine-1-carboxylate (0.40 g).

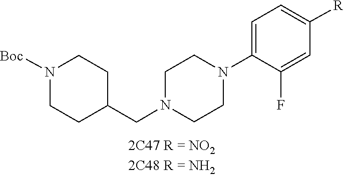

Int. 3E5 (1.07 g), 3,4-difluoronitrobenzene (0.50 g), and K₂CO₃ (0.65 g) were stirred ON in DMF (5 mL) at 80° C. The OL (EtOAc/water) was dried, concentrated, and purified by FC (pentane/EtOAc 1:0 to 1:4) to give Int. 2C47 tert-butyl 4-[[4-(2-fluoro-4-nitro-phenyl)-piperazin-1-yl]methyl]-piperidine-1-carboxylate (0.47 g). Int. 2C47 (0.44 g), NH₄Cl (0.28 g), and iron powder (0.29 g) were stirred 2 h in THF/EtOH/water (4:4:3, 11 mL) at 80° C., filtered, concentrated, and purified by FC (hexane/EtOAc 1:0 to 1:1) to give Int. 2C48 tert-butyl 4-[[4-[4-[(2,6-dioxo-3-piperidyl)-amino]-2-fluoro-phenyl]-piperazin-1-yl]-methyl]piperidine-1-carboxylate (0.30 g).

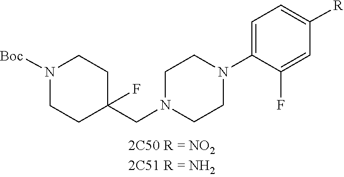

Int. 2C50 (0.63 g) was prepared from 1,2-difluoro-4-nitrobenzene and Int. 3E143 similarly to Int. 2I4. Int. 2C49 3-((3-fluoro-4-(4-((4-fluoropiperidin-4-yl)methyl)-piperazin-1-yl)phenyl)amino)-piperidine-2,6-dione (0.17 g) was prepared from Int. 2C50 similarly to Int. 2N5. Int. 2C51 was prepared from 1,2-difluoro-4-nitrobenzene and Int. 3E143 similarly to Int. 2C48.

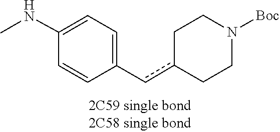

PdDPPFCl₂-DCM (0.22 g), tert-butyl 4-((4,4,5,5-tetramethyl-1,3,2-dioxaborolan-2-yl)methylene)piperidine-1-carboxylate (0.87 g), and 4-bromo-N-methylaniline (0.50 g) were degassed in dioxane/10% aq. Na₂CO₃ (1:1.2, 12.4 mL) and stirred 2 h at 100° C. The OL (EtOAc/brine) was dried, concentrated, and purified by FC (heptane/EtOAc 1:0 to 0:1) to give Int. 2C59 tert-butyl 4-(4-(methylamino)-benzylidene)piperidine-1-carboxylate (0.65 g). Int. 2C59 (0.53 g) was hydrogenated 2 h using 10% Pd/C (0.1 g) in MeOH (8.7 mL), filtered, concentrated, and purified by FC (heptane/EtOAc 1:0 to 0:1) to give Int. 2C58 tert-butyl 4-(4-(methylamino)benzyl)piperidine-1-carboxylate (1.0 g).

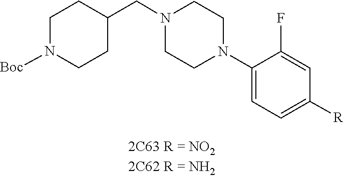

3,4-Difluoro-nitrobenzene (4.0 g), Int. 3E5 (8.6 g), and K₂CO₃ (5.2 g) were stirred ON in DMF (5 mL) at 80° C. The OL (water/EtOAc) was washed with brine, dried, concentrated, and purified by FC (hexane/EtOAc 1:0 to 4:1) to give Ind 2C63 tert-butyl 4-((4-(2-fluoro-4-nitrophenyl)piperazin-1-yl)methyl)piperidine-1-carboxylate (6.4 g). Int. 2C63 (8.0 g), iron powder (5.3 g), and NH₄Cl (5.1 g) were stirred 2 h in THF/EtOH/water (1.6:1.6:1, 104 mL) at 80° C. The mixture was filtered, concentrated, and purified by FC (hexane/EtOAc 4:1 to 1:1) to give Int. 2C62 tert-butyl 4-[[4-(4-amino-2-fluoro-phenyl)-piperazin-1-yl]methyl]piperidine-1-carboxylate (6.0 g).

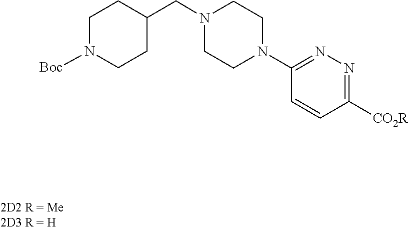

Methyl 6-chloropyridazine-3-carboxylate (0.5 g) was stirred 0.25 h in DMSO/DIPEA (2:1, 4.5 mL). Int. 3E5 (821 g) was added and stirring continued ON at 120° C. The OL (EtOAc/water) was concentrated and purified by FC (pentane/EtOAc 4:1) to give Int. 2D2 methyl 6-(4-((1-(tert-butoxy-carbonyl)piperidin-4-yl)-methyl)-piperazin-1-yl)pyridazine-3-carboxylate (0.7 g). Int. 2D2 (0.8 g) and LiOH—H₂O (0.4 g) were stirred 6 h in THF/MeOH/water (2:2:1, 8 mL) and concentrated. The OL (DCM/MeOH (9:1)/water) was concentrated and purified by FC (hexane/EtOAc 1:1) to give Int. 2D3 6-(4-((1-(tert-butoxycarbonyl)-piperidin-4-yl)-methyl)-piperazin-1-yl)-pyridazine-3-carboxylic acid (0.65 g).

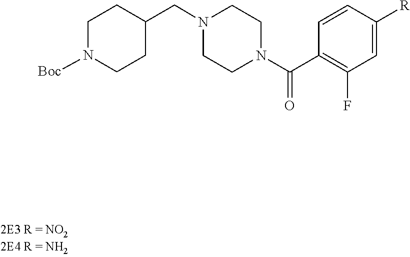

2-Fluoro-4-nitro-benzoic acid (0.16 g), Int. 3E5 (0.20 g), and HATU (322 mg) were stirred ON in DMF/DIPEA (6:1, 3.6 mL). The OL (EtOAc/water) was concentrated. The residue was purified by FC (EtOAc/MeOH 1:0 to 95:5) to give Int. 2E3 tert-butyl 4-((4-(2-fluoro-4-nitrobenzoyl)piperazin-1-yl)methyl)piperidine-1-carboxylate (0.19 g). This material and iron powder (0.2 g) were stirred 0.75 h in EtOH/water (1:1, 3 mL) and AcOH (0.05 mL) at 80° C. and filtered and concentrated. The OL (DCM/water) was washed with sat. aq. NaHCO₃ and brine, concentrated, and purified by SCX give Int. 2E4 tert-butyl 4-((4-(4-amino-2-fluorobenzoyl)-piperazin-1-yl)-methyl)piperidine-1-carboxylate (0.18 g).

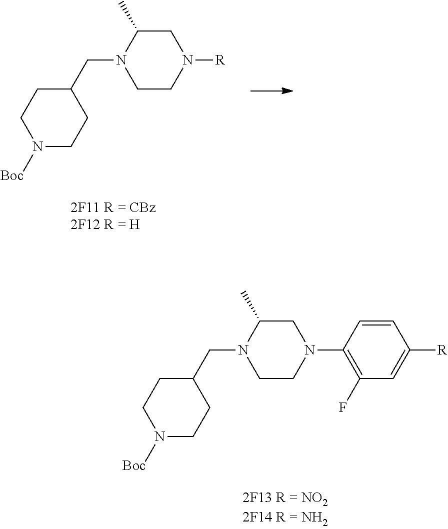

Benzyl (R)-3-methylpiperazine-1-carboxylate (12 g, HCl salt) and tert-butyl 4-formyl-piperidine-1-carbo-xylate (11 g) were stirred 2 h in DCE/DIPEA (5:1, 240 mL) at 0° C. to RT. STAB (19 g) was added and stirring continued ON before concentration. The OL (water/DCM) was concentrated and purified by FC (hexane/EtOAc 4:1 to 3:2) to give Int. 2F11 benzyl (R)-4-((1-(tert-butoxycarbonyl)piperidin-4-yl)methyl)-3-methyl-piperazine-1-carboxylate (12 g). Int. 2F11 (5 g) and 10% Pd/C (1.5 g) were stirred ON in EtOAc/THF (2:1, 75 mL) under a H₂ atmosphere (70 psi). The filtrate after concentration was dried to give Int. 2F12 tert-butyl (R)-4-((2-methylpiperazin-1-yl)-methyl)-piperidine-1-carboxylate (3.1 g). Int. 2F12 (9.0 g), K₂CO₃ (12 g) and 1,2-difluoro-4-nitrobenzene (6 g) were stirred 4 h in DMF (50 mL) at 0-80° C. and filtered. The OL (EtOAc/water) was concentrated and purified by FC (pentane/EtOAc 7:3 to 3:2) to give Int. 2F13 tert-butyl (R)-4-((4-(2-fluoro-4-nitrophenyl)-2-methyl-piperazin-1-yl)methyl)-piperidine-1-carboxylate (4.5 g). Int. 2F13 (4.2 g), NH₄Cl (2.5 g), and iron powder (2.7 g) were stirred 6 h in THF/EtOH/water (2:2:1, 62 mL) at 80° C. and concentrated. The OL (water/DCM) was concentrated and purified by FC (hexane/EtOAc 3:1 to 3:2) to give Int. 2F14 tert-butyl (R)-4-((4-(4-amino-2-fluoro-phenyl)-2-methylpiperazin-1-yl)methyl)-piperidine-1-carboxylate (3.8 g).

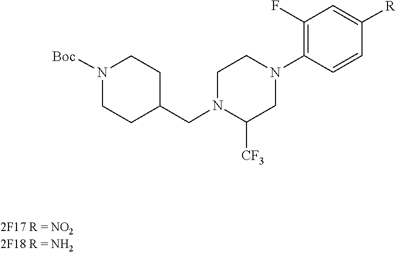

Int. 3E139 (0.36 g), 1,2-difluoro-4-nitrobenzene (162 mg), and K₂CO₃ (0.42 g) were stirred 48 h at 80° C. in ACN (5 mL). The OL (EtOAc/water) was concentrated and purified by FC (hexane/EtOAc 7:3) to give Int. 2F17 tert-butyl 4-((4-(2-fluoro-4-nitrophenyl)-2-(trifluoro-methyl)piperazin-1-yl)methyl)piperidine-1-carboxylate (0.30 g). Int. 2F17 (0.1 g) was hydrogenated ON using 10% Pd/C (20 mg) in THF/dioxane (3:1, 8 mL), filtered, and concentrated to give Int. 2F18 tert-butyl 4-((4-(4-amino-2-fluorophenyl)-2-(trifluoromethyl)piperazin-1-yl)methyl)-piperidine-1-carboxylate (50 mg).

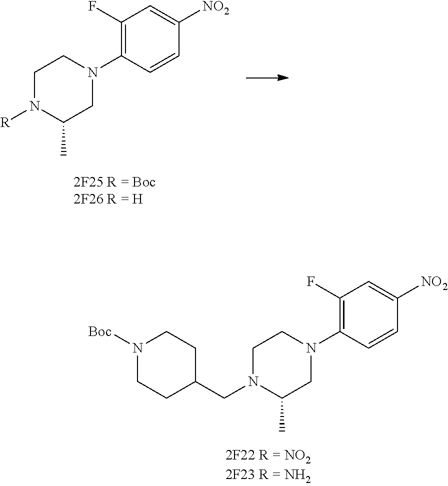

K₂CO₃ (1.36 g), 1,2-difluoro-4-nitro-benzene (0.52 g), and tert-butyl (S)-2-methyl-piperazine-1-carboxylate (0.65 g) were stirred ON in DMF (3 mL) at 70° C. The OL (EtOAc/water) was washed with 3M aq. CaCl₂ and brine, concentrated, and purified by FC (heptane/EtOAc 1:0 to 3:1) to give Int. 2F25 tert-butyl (S)-4-(2-fluoro-4-nitro-phenyl)-2-methylpiperazine-1-carboxylate (0.95 g). This material was stirred 1.5 h in 2.7M HCl in dioxane (15 mL) and concentrated to give Int. 2F26 (S)-1-(2-fluoro-4-nitro-phenyl)-3-methylpiperazine (0.77 g, HCl salt). Int. 2F26 (0.68 g) tert-butyl 4-formylpiperidine-1-carboxylate (0.96 g) were stirred 0.25 h in DMF/DIPEA (5.3:1, 12 mL). STAB (2.4 g) was added and stirring continued ON. The OL (EtOAc/water) was washed with 3M aq. CaCl₂ and brine, concentrated, and purified by FC (heptane/EtOAc 1:0 to 3:2) to give Int. 2F22 tert-butyl (S)-4-((4-(2-fluoro-4-nitrophenyl)-2-methyl-piperazin-1-yl)methyl)-piperidine-1-carboxylate (0.74 g).

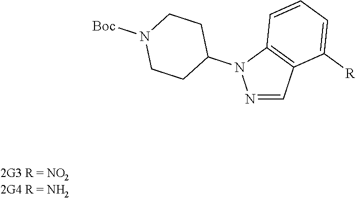

tert-Butyl 4-((methylsulfonyl)oxy)piperidine-1-carboxylate (0.38 g) was added over 5 h to a mixture of 4-nitro-1H-indazole (0.20 g) and K₂CO₃ (0.34 g) in DMF (6 mL) at 100° C. and stirring was continued ON. The OL (EtOAc/water) was concentrated and purified by FC (heptane/EtOAc 1:0 to 0:1) to give Int. 2G3 tert-butyl 4-(4-nitro-indazol-1-yl)-piperidine-1-carboxylate (0.42 g). Int. 2G3 (0.42 g) was hydrogenated 4 h using 10% Pd/C (50 mg) in EtOH (4 mL), filtered, concentrated, and HPLC-purified to give Int. 2G4 tert-butyl 4-(4-aminoindazol-1-yl)-piperidine-1-carboxylat (63 mg).

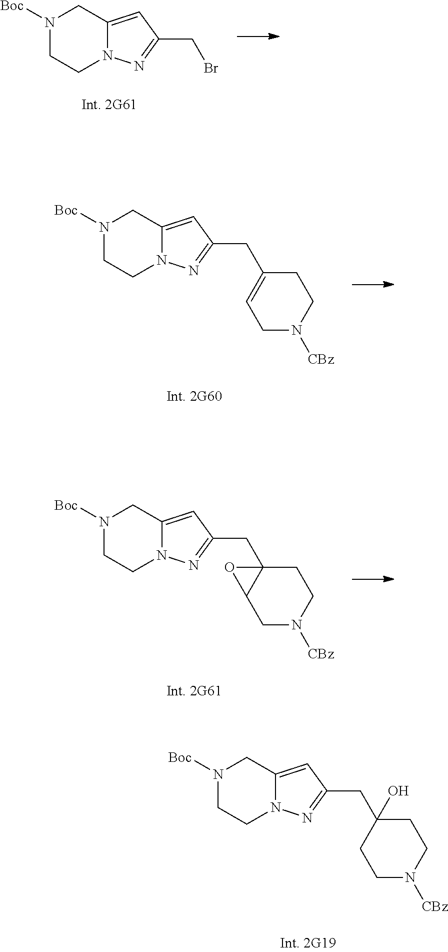

CBr₄ (15.7 g), PPh₃ (15.5 g), and tert-butyl 2-(hydroxymethyl)-6,7-dihydropyrazolo[1,5-a]pyrazine-5 (4H)-carboxylate (10.0 g) were stirred 16 h in DCM (80 mL). The OL (water/DCM) was dried, filtered, concentrated and purified by FC (hexane/EtOAc 2:1) to give Int. 2G61 tert-butyl 2-(bromomethyl)-6,7-dihydropyrazolo[1,5-a]pyrazine-5 (4H)-carboxylate (10.0 g). Int. 2G61 (10.0 g), 1-phenyl-2-(4-(4,4,5,5-tetramethyl-1,3,2-dioxaborolan-2-yl)-3,6-dihydropyridin-1 (2H)-yl)-212-ethan-1-one (10.9 g), K₂CO₃ (13.1 g) and PdDPPFCl₂-DCM (2.6 g) were stirred 16 h in dioxane/H₂O (7:3, 100 mL) under an inert atmosphere. The mixture was filtered. The OL (water/EtOAc) was dried, filtered, concentrated, and purified by FC (hexane/EtOAc 2:1) to give Int. 2G60 tert-butyl 2-((1-((benzyloxy)carbonyl)-1,2,3,6-tetrahydropyridin-4-yl)methyl)-6,7-dihydropyrazolo[1,5-a]pyrazine-5 (4H)-carboxylate (7.0 g). m-CPBA (23 g) and Int. 2G60 (30 g) were stirred 4 h in DCM (200 mL) at 0° C. to RT. The OL (aq. NaHCO₃/DCM) was dried, filtered and concentrated to give Int. 2G59 benzyl 6-((5-(tert-butoxycarbonyl)-4,5,6,7-tetrahydropyrazolo[1,5-a]pyrazin-2-yl)methyl)-7-oxa-3-azabicyclo[4.1.0]heptane-3-carboxylate (28 g). Int. 2G59 (17 g) and Pd/C 10% wt. (7 g) were hydrogenated at 60 psi in dioxane (170 mL), filtered, concentrated and HPLC-purified to give Int. 2G19 tert-butyl 2-((4-hydroxypiperidin-4-yl)methyl)-6,7-dihydropyrazolo[1,5-a]pyrazine-5 (4H)-carboxylate (11 g).

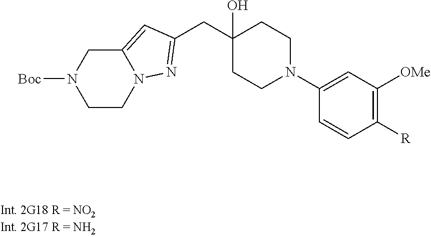

K₂CO₃ (7.3 g), Int. 2G19 (6.5 g), and 4-fluoro-2-methoxy-1-nitrobenzene (3 g) were stirred 15 h in DMF (30 mL) at 70° C. The OL (water/DCM/MeOH) was dried, filtered, concentrated, and purified by FC (EtOAc) to give Int. 2G18 tert-butyl 2-((4-hydroxy-1-(3-methoxy-4-nitrophenyl)piperidin-4-yl)methyl)-6,7-dihydropyrazolo[1,5-a]pyrazine-5 (4H)-carboxylate (3.1 g). Int. 2G18 (2.5 g) was hydrogenated for 16 h using 10% Pd/C (1.2 g) in MeOH (30 mL), filtered, and concentrated to give Int. 2G17 tert-butyl 2-((1-(4-amino-3-methoxyphenyl)-4-hydroxy-piperidin-4-yl)methyl)-6,7-dihydropyrazolo[1,5-a]-pyrazine-5 (4H)-carboxylate (1.7 g).

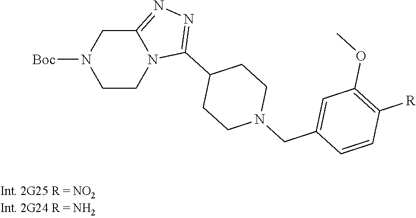

3-Methoxy-4-nitrobenzaldehyde (0.32 g), tert-butyl 3-(piperidin-4-yl)-5,6-dihydro-[1,2,4]triazolo[4,3-a]pyrazine-7 (8H)-carboxylate (0.50 g), and NaCNBH₃ (0.31 g) were stirred ON in DCE/Et₃N (15:1, 10.7 mL). The mixture was filtered, concentrated, and purified by FC (MTBE/MeOH) to give Int. 2G25 tert-butyl 3-(1-(3-methoxy-4-nitrobenzyl)piperidin-4-yl)-5,6-dihydro-[1,2,4]triazolo[4,3-a]pyrazine-7 (8H)-carboxylate (0.30 g). Int. 2G25 (0.30 g) was hydrogenated ON using Pt/C 10% wt. (50 mg) in MeOH (10 mL), filtered, and concentrated to give Int. 2G24 tert-butyl 3-(1-(4-amino-3-methoxybenzyl)-piperidin-4-yl)-5,6-dihydro-[1,2,4]triazolo[4,3-a]pyrazine-7 (8H)-carboxylate (0.15 g).

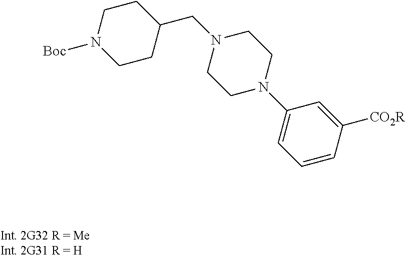

Methyl 3-bromobenzoate (3 g), Cs₂CO₃ (9.1 g), BINAP (1.7 g), Pd(OAc)₂ (0.31 g), and tert-butyl 4-(piperazin-1-ylmethyl)piperidine-1-carboxylate were stirred 16 h in dioxane (20 mL) at 100° C. under an inert atmosphere. The mixture was filtered, concentrated, and purified by FC (hexane/EtOAc 3:2) to give Int. 2G32 tert-butyl 4-((4-(3-(methoxycarbonyl)phenyl)piperazin-1-yl)methyl)piperidine-1-carboxylate (3.3 g). LiOH·H₂O (2.4 g) and Int. 2G32 (4 g) were stirred 16 h in THF/MeOH/H₂O (2:1:1, 40 mL) at 0° C. to RT. The mixture was concentrated. The OL (aq. 10% citric acid/EtOAc) was concentrated to give Int. 2G31 3-(4-((1-(tert-butoxycarbonyl)piperidin-4-yl)-methyl)-piperazin-1-yl)benzoic acid (3.3 g).

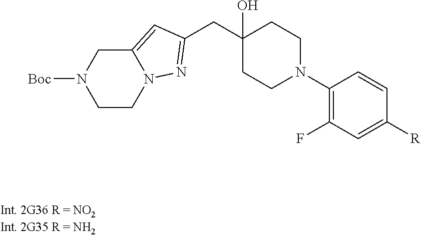

K₂CO₃ (0.22 g), Int. 2G19 (0.20 g), and 1,2-difluoro-4-nitrobenzene (91 mg) were stirred ON in ACN (2 mL) at 80° C. The mixture was filtered, concentrated, and HPLC-purified to give Int. 2G36 tert-butyl 2-((1-(2-fluoro-4-nitrophenyl)-4-hydroxypiperidin-4-yl)methyl)-6,7-dihydropyrazolo-[1,5-a]pyrazine-5 (4H)-carboxylate (69 mg). Int. 2G36 (69 mg) and Pd/C (50 mg) were hydrogenated ON in MeOH (5 mL), filtered, and concentrated to give Int. 2G35 tert-butyl 2-((1-(4-amino-2-fluoro-phenyl)-4-hydroxypiperidin-4-yl)methyl)-6,7-dihydropyrazolo[1,5-a]pyrazine-5 (4H)-carboxylate (65 mg).

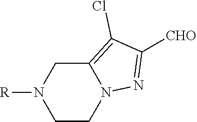

NCS (0.53 g) and tert-butyl 2-formyl-6,7-dihydropyrazolo[1,5-a]pyrazine-5 (4H)-carboxylate (0.50 g) were stirred 16 h in DMF (10 mL). The OL (brine/EtOAc) was dried, filtered and concentrated to give Int. 2G39 tert-butyl 3-chloro-2-formyl-6,7-dihydropyrazolo[1,5-a]pyrazine-5 (4H)-carboxylate (0.54 g).

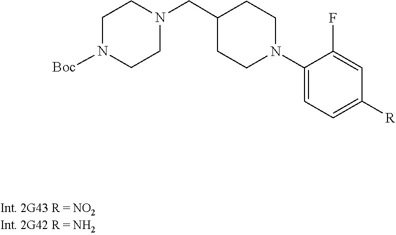

1,2-Difluoro-4-nitrobenzene (1.1 g), tert-butyl 4-(piperidin-4-ylmethyl)piperazine-1-carboxylate (2.0 g), and K₂CO₃ (2.9 g) were stirred ON in DMF (50 mL) at 100° C. under an inert atmosphere. The OL (water/EtOAc) dried, filtered, and concentrated to give Int. 2G43 tert-butyl 4-((1-(2-fluoro-4-nitro-phenyl)piperidin-4-yl)methyl)piperazine-1-carboxylate (2.2 g). Int. 2G43 (2.2 g) was hydrogenated ON using Pd/C (55 mg) in MeOH (50 mL), filtered, and concentrated to give Int. 2G42 tert-butyl 4-((1-(4-amino-2-fluorophenyl)piperidin-4-yl)methyl)piperazine-1-carboxylate (1.5 g).

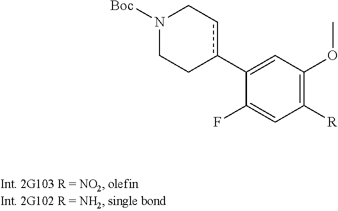

1-Bromo-2-fluoro-5-methoxy-4-nitrobenzene (5 g), N-Boc-DHP-4-boronic acid pinacol ester (6.5 g) and K₂CO₃ (9 g) were mixed in dioxane/H₂O (40:1, 10 mL). PdDPPFCl₂-DCM (1.5 g) was added and the mixture stirred for 12 h at 80° C. under an atmosphere of argon. The mixture was filtered and concentrated to give crude Int. 2G103 tert-butyl 4-(2-fluoro-5-methoxy-4-nitrophenyl)-3,6-dihydropyridine-1 (2H)-carboxylate (7.4 g). Int. 2G103 (7.2 g) was hydrogenated for 12 h using Pd/C (0.5 g) in MeOH (100 mL), filtered, and concentrated to give Int. 2G102 tert-butyl 4-(4-amino-2-fluoro-5-methoxyphenyl)piperidine-1-carboxylate (6.0 g).

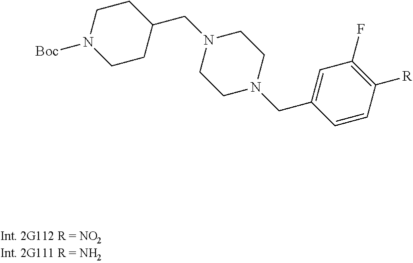

STAB (10 g), 3-fluoro-4-nitrobenzaldehyde (2 g), and tert-butyl 4-(piperazin-1-ylmethyl)piperidine-1-carboxylate (3.3 g) were stirred 12 h in DCE (60 mL). The OL (water/DCE) was concentrated to give Int. 2G112 tert-butyl 4-((4-(3-fluoro-4-nitrobenzyl)piperazin-1-yl)methyl)-piperidine-1-carboxylate (1.9 g). Int. 2G112 (1.9 g) and Pd/C (0.30 g) were hydrogenated in MeOH (30 mL), filtered, and concentrated to give Int. 2G111 tert-butyl 4-((4-(4-amino-3-fluorobenzyl)-piperazin-1-yl)methyl)-piperidine-1-carboxylate (1.5 g).

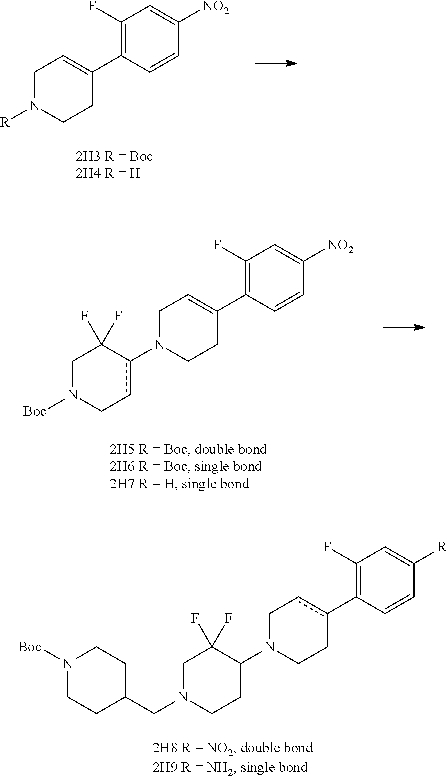

1-Bromo-2-fluoro-4-nitrobenzene (6 g), PdCl₂(PPh₃)₂ (1.0 g), and tert-butyl 4-(4,4,5,5-tetramethyl-1,3,2-dioxa-borolan-2-yl)-3,6-dihydropyridine-1 (2H)-carboxylate (10 g) were degassed in dioxane/water (12:1, 65 mL) and stirred ON at 90° C. The OL (EtOAc/water) was concentrated and purified by FC (pentane/EtOAc 7:3 to 65:35) to give Int. 2H3 tert-butyl 4-(2-fluoro-4-nitrophenyl)-3,6-dihydro-pyridine-1 (2H)-carboxylate (8 g). This material was stirred 2 h in 2M HCl in 1,4-dioxane (160 mL) at 0° C. to RT, concentrated, and triturated in pentane to give Int. 2H4 4-(2-fluoro-4-nitro-phenyl)-1,2,3,6-tetrahydropyridine (5 g, HCl salt). Int. 2H4 (6.0 g) and NaOAc (4.8 g) were stirred 0.5 h in ACN/toluene (1:2, 150 mL). tert-Butyl 3,3-difluoro-4-oxo-piperidine-1-carboxylate (8.7 g) and AcOH (5 mL) were added and stirring continued ON at 100° C. The filtrate after filtration was concentrated to give Int. 2H5 tert-butyl 3′,3′-difluoro-4-(2-fluoro-4-nitro-phenyl)-3,3′,6,6′-tetrahydro-2H-[1,4′-bipyridine]-1′(2′H)-carboxylate (7 g). This material and NaCNBH₃ (5.0 g) were stirred ON in MeOH/DCE/AcOH (6.7:6.7:1, 43 mL). The residue after concentration was diluted with EtOAc and filtered. The residue after concentration of the filtrate was purified by FC (pentane/EtOAc 1:0 to 7:3) to give Int. 2H6 tert-butyl 3,3-difluoro-4-(4-(2-fluoro-4-nitro-phenyl)-3,6-dihydropyridin-1 (2H)-yl)piperidine-1-carboxylate (3 g). Int. 2H6 (1.5 g) was stirred 2 h in 2M HCl in dioxane (20 mL) at 0° C. to RT. The mixture was concentrated to give Int. 2H7 1-(3,3-difluoro-piperidin-4-yl)-4-(2-fluoro-4-nitrophenyl)-1,2,3,6-tetrahydropyridine (1.2 g, HCl salt). This material and tert-butyl 4-formyl-piperidine-1-carboxylate (0.81 g) were stirred 4 h in DCE/DIPEA (10:1, 17 mL). STAB (1.4 g) was added and stirring was continued ON at 0° C. to RT. The OL (water/DCM) was concentrated and purified by FC (pentane/EtOAc 1:1 to 2:3) to give Int. 2H8 tert-butyl 4-((3,3-difluoro-4-(4-(2-fluoro-4-nitro-phenyl)-3,6-dihydropyridin-1 (2H)-yl)piperidin-1-yl)methyl)-piperidine-1-carboxylate (1.1 g). This material was hydrogenated ON using 50% Pd/C (0.5 g) in EtOAc (6 mL), filtered, and concentrated to give Int. 2H9 tert-butyl 4-((4-(4-amino-2-fluoro-phenyl)-3′,3′-difluoro-[1,4′-bipiperidin]-1′-yl)-methyl)piperidine-1-carboxylate (1.0 g).

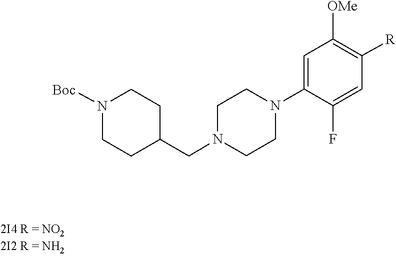

1,2-Difluoro-4-methoxy-5-nitro-benzene (2.7 g), tert-butyl 4-(piperazin-1-ylmethyl)-piperidine-1-carboxylate (4.9 g), and K₂CO₃ (2.9 g) were stirred ON in DMF (10 mL) at 80° C. The OL (EtOAc/water) was washed with brine, dried, concentrated, and purified by FC (pentane/EtOAc 4:1 to 7:3) to give Int. 2I4 tert-butyl 4-((4-(2-fluoro-5-methoxy-4-nitrophenyl)piperazin-1-yl)methyl)piperidine-1-carboxylate (4 g).

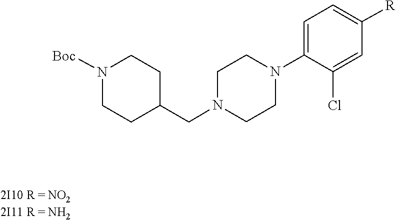

Int. 2I10 tert-butyl 4-((4-(2-fluoro-5-methoxy-4-nitrophenyl)piperazin-1-yl)methyl)-piperidine-1-carboxylate (1.2 g) prepared similarly to Int. 2I4 from 2-chloro-1-fluoro-4-nitrobenzene (1.8 g).

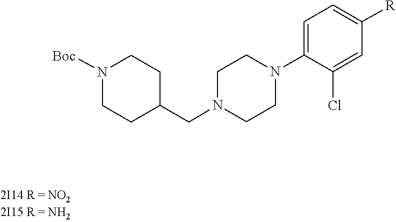

Int. 2I14 tert-butyl 4-((4-(2-fluoro-5-methoxy-4-nitrophenyl)piperazin-1-yl)methyl)piperidine-1-carboxylate (2 g) prepared similarly to Int. 2I4 from 2-fluoro-5-nitrobenzonitrile (1.0 g).

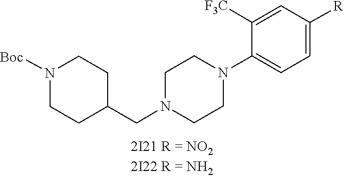

Int. 2I21 tert-butyl 4-((4-(4-nitro-2-(trifluoromethyl)phenyl)piperazin-1-yl)methyl)-piperidine-1-carboxylate (4 g) was prepared from 1-fluoro-4-nitro-2-(trifluoromethyl)-benzene (2.5 g) similarly to Int. 2I4.

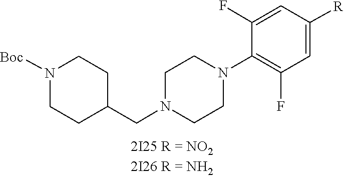

Int. 2I25 tert-butyl 4-((4-(2,6-difluoro-4-nitrophenyl)piperazin-1-yl)methyl)piperidine-1-carboxylate (0.45 g) prepared similarly to Int. 2I4 from 1,2,3-trifluoro-5-nitro-benzene.

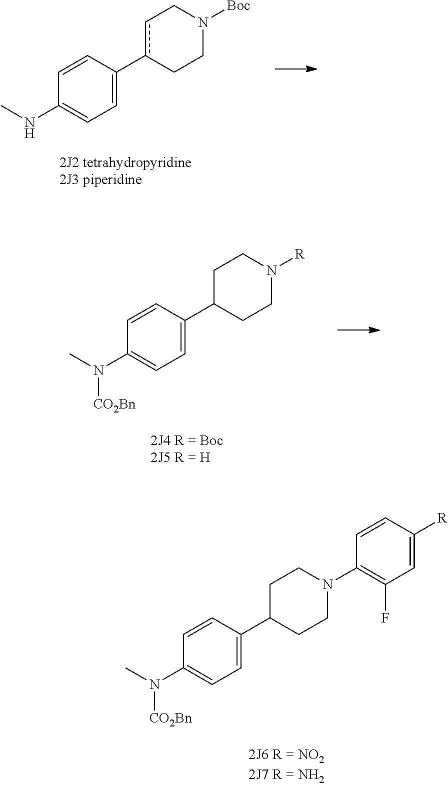

4-Bromo-N-methylaniline (2.4 g), Cs₂CO₃ (13 g), tert-butyl 4-(4,4,5,5-tetramethyl-1,3,2-dioxaborolan-2-yl)-3,6-dihydro-pyridine-1 (2H)-carboxylate (4.8 g), and PdDPPFCl₂-DCM (0.5 g) were degassed in dioxane/water (10:1, 44 mL) and stirred ON at 110° C., filtered, concentration, and purified by FC (heptane/EtOAc 1:0 to 4:1) to give Int. 2J2 tert-butyl 4-(4-(methylamino)phenyl)-3,6-dihydropyridine-1 (2H)-carboxylate (2.7 g). This material was hydrogenated ON using 10% Pd/C (0.5 g) in MeOH (37 mL), filtered, and concentrated to give Int. 2J3 tert-butyl 4-(4-(methyl-amino)phenyl)-piperidine-1-carboxylate (2.6 g). Int. 2J3 (0.83 g) was stirred in DCM/DIPEA (4:1, 7.5 mL) at 0° C. CBz-Cl (0.55 g) was added and stirring was continued ON at 0° C. to RT. The OL (DCM/water) was washed with brine, concentrated, and purified by FC (hexane/EtOAc 1:0 to 3:2) to give Int. 2J4 tert-butyl 4-(4-(((benzyloxy)-carbonyl)(methyl)-amino)phenyl)-piperidine-1-carboxylate (1.1 g). This material was stirred 1 h in 2.7M HCl in dioxane (15 mL) and concentrated to give Int. 2J5 benzyl methyl(4-(piperidin-4-yl)phenyl)-carbamate (0.79 g, HCl salt). Int. 2J5 (0.25 g), K₂CO₃ (0.29 g), and 1,2-difluoro-4-nitrobenzene (0.11 g) were stirred ON at 70° C. and 48 h at RT. The OL (EtOAc/water) was washed with brine, concentrated, and HPLC-purified to give Int. 2J6 benzyl (4-(1-(2-fluoro-4-nitrophenyl)-piperidin-4-yl)phenyl)(methyl)-carbamate (0.27 g). This material and iron powder (0.33 g) were stirred ON in THF/AcOH (2:1, 9 mL). Iron powder (0.33 g) was added and stirring continued 4 h. The filtrate was SCX-purified to give Int. 2J7 benzyl (4-(1-(4-amino-2-fluoro-phenyl)-piperidin-4-yl)phenyl)(methyl)-carbamate (0.25 g).

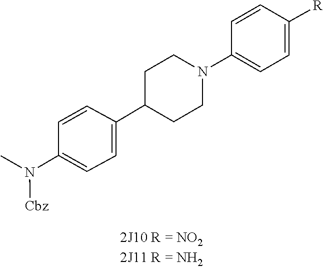

Int. 2J11 was prepared similarly to Int. 2J7 from 1-fluoro-4-nitrobenzene.

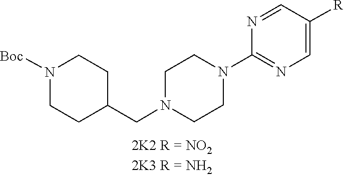

Int. 2K2 tert-butyl 4-((4-(5-nitropyrimidin-2-yl)piperazin-1-yl)methyl)piperidine-1-carboxylate (0.2 g) was prepared from 2-chloro-5-nitropyrimidine (0.10 g) similarly to Int. 2I4.

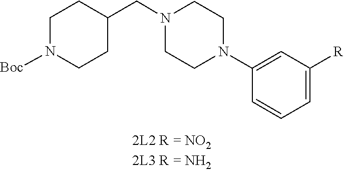

1-Bromo-3-nitrobenzene (0.30 g), tert-butyl 4-(piperazin-1-ylmethyl)piperidine-1-carboxylate (0.51 g), NaOtBu (214 mg), XantPhos (86 mg), and Pd₂dba₃ (136 mg) were degassed in toluene (12 mL) and stirred 48 h at 130° C. Filtration, concentration, and HPLC-purification gave Int. 2L2 tert-butyl 4-((4-(3-nitro-phenyl)-piperazin-1-yl)-methyl)piperidine-1-carboxylate (0.24 g). This material was hydrogenated ON using 10% Pd/C (0.1 g) in EtOH (10 mL), filtered, and concentrated to give Int. 2L3 tert-butyl 4-((4-(3-amino-phenyl)-piperazin-1-yl)methyl)-piperidine-1-carboxylate (0.20 g).

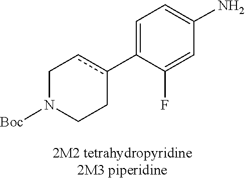

4-Bromo-3-fluoro-aniline (0.22 g), tert-butyl 4-(4,4,5,5-tetramethyl-1,3,2-dioxaborolan-2-yl)-3,6-dihydro-2H-pyridine-1-carboxylate (0.43 g), K₂CO₃ (0.48 g), and PdPDDFCl₂-DCM (42 mg) were degassed in dioxane/water (6:1, 7 mL) and stirred ON at 90° C., filtered, concentrated and purified by FC (heptane/EtOAc 1:0 to 65:35) to give Int. 2M2 tert-butyl 4-(4-amino-2-fluorophenyl)-3,6-dihydropyridine-1 (2H)-carboxylate (0.28 g). This material was hydrogenated ON using 10% Pd/C (50 mg) in EtOAc (6 mL), filtered, and concentrated to give Int. 2M3 tert-butyl 4-(4-amino-2-fluorophenyl)-piperidine-1-carboxylate (0.27 g).

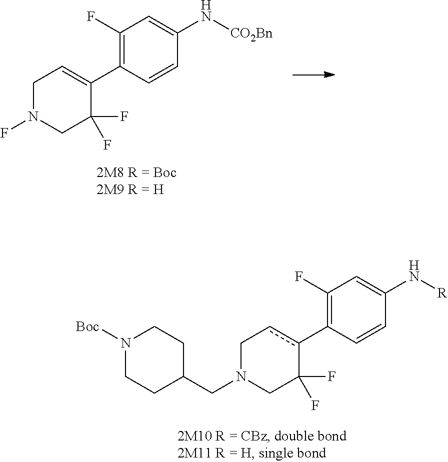

Na₂CO₃ (0.96 g), benzyl (3-fluoro-4-(4,4,5,5-tetramethyl-1,3,2-dioxaborolan-2-yl)phenyl)carbamate (1.4 g), Int. 3E284 (1.7 g), and PdPDDFCl₂-DCM (0.28 g) were degassed in dioxane/water (5:1, 24 mL) and stirred ON at 60° C. The OL (EtOAc/water) was washed with brine, dried, concentrated, and purified by FC (hexane/EtOAc 4:1 to 7:3) to give Int. 2M8 tert-butyl 4-(4-(((benzyloxy)carbonyl)amino)-2-fluorophenyl)-3,3-difluoro-3,6-dihydro-pyridine-1 (2H)-carboxylate (1.5 g). This material (1.1 g) was stirred ON in DCM/4M HCl in dioxane (1:1, 22 mL) at 0° C. to RT, concentrated, and triturated in Et₂O/pentane to give Int. 2M9 benzyl (4-(3,3-difluoro-1,2,3,6-tetrahydropyridin-4-yl)-3-fluoro-phenyl)-carbamate (0.76 g, HCl salt).

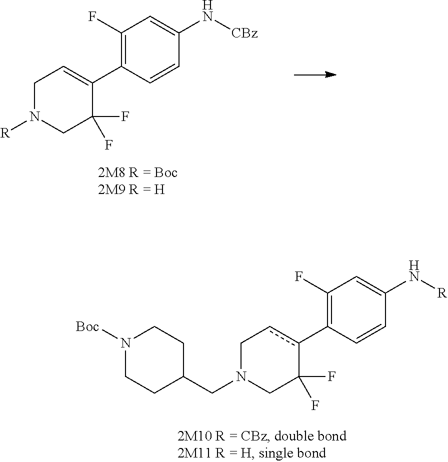

Na₂CO₃ (0.96 g), benzyl (3-fluoro-4-(4,4,5,5-tetramethyl-1,3,2-dioxaborolan-2-yl)-phenyl)carbamate (1.4 g), Int. 3E284 (1.7 g), and PdPDDFCl₂-DCM (0.28 g) were degassed in dioxane/water (5:1, 24 mL) and stirred ON at 60° C. The OL (EtOAc/water) was washed with brine, dried, concentrated, and purified by FC (hexane/EtOAc 4:1 to 7:3) to give Int. 2M8 tert-butyl 4-(4-(((benzyloxy)carbonyl)amino)-2-fluorophenyl)-3,3-difluoro-3,6-dihydro-pyridine-1 (2H)-carboxylate (1.5 g). This material (1.1 g) was stirred ON in DCM/4M HCl in dioxane (1:1, 22 mL) at 0° C. to RT, concentrated, and triturated in Et₂O/pentane to give Int. 2M9 benzyl (4-(3,3-difluoro-1,2,3,6-tetrahydro-pyridin-4-yl)-3-fluoro-phenyl)-carbamate (0.76 g, HCl salt). This material (0.73 g) and tert-butyl 4-formylpiperidine-1-carboxylate (0.39 g) were stirred 3 h in DCE/DIPEA (10:1, 19.6 mL) at 0° C. to RT. STAB (0.78 g) was added and stirring continued ON before concentration. The OL (water/MeOH/DCM (1:9)) was washed with brine, dried, concentrated, and purified by FC (hexane/EtOAc 3:2 to 1:1) to give Int. 2M10 tert-butyl 4-((4-(4-(((benzyloxy)-carbonyl)amino)-2-fluorophenyl)-3,3-difluoro-3,6-dihydropyridin-1 (2H)-yl)methyl)-piperidine-1-carboxylate (0.70 g). This material was hydrogenated 48 h using 10% Pd/C (0.25 g) in EtOAc (15 mL), filtered, concentrated, and purified by FC (hexane/EtAOC 1:4 to 0:1) to give Int. 2M11 tert-butyl 4-((4-(4-amino-2-fluorophenyl)-3,3-difluoropiperidin-1-yl)methyl)piperidine-1-carboxylate (0.21 g).

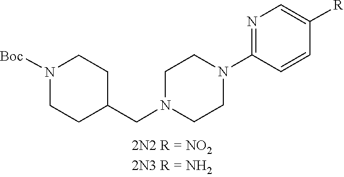

Int. 2N2 tert-butyl 4-((4-(5-nitropyridin-2-yl)piperazin-1-yl)methyl)piperidine-1-carboxylate (0.76 g) was prepared from 2-chloro-5-nitropyridine (0.90 g) similarly to Int. 2I4.

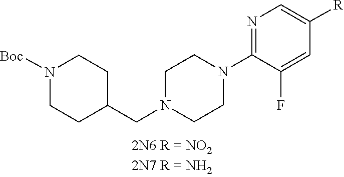

Int. 2N6 tert-butyl 4-((4-(3-fluoro-5-nitropyridin-2-yl)piperazin-1-yl)methyl)piperidine-1-carboxylate (4.5 g) was prepared from 2-chloro-3-fluoro-5-nitropyridine (2.5 g) similarly to Int. 2I4.

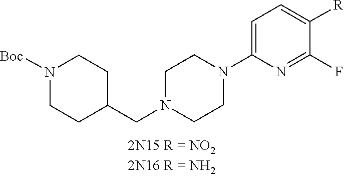

Int. 2N15 tert-butyl 4-((4-(6-fluoro-5-nitropyridin-2-yl)piperazin-1-yl)methyl)piperidine-1-carboxylate (1.0 g) was prepared from 2,6-difluoro-3-nitropyridine similarly to Int. 2I4 using DIPEA instead of K₂CO₃.

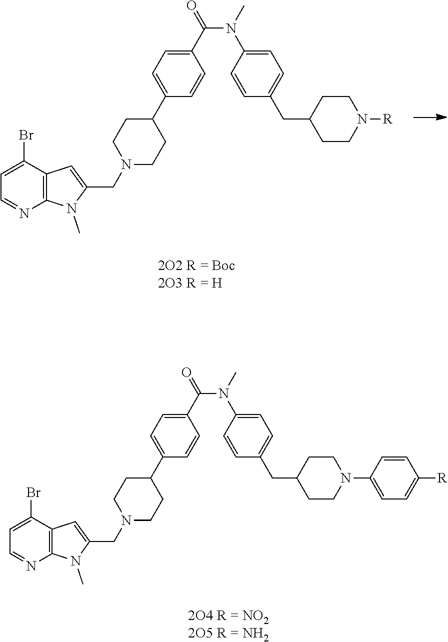

Int. 2O2 tert-butyl 4-(4-(4-(1-((4-bromo-1-methyl-1H-pyrrolo[2,3-b]pyridin-2-yl)methyl)-piperidin-4-yl)-N-methylbenzamido)benzyl)piperidine-1-carboxylate (357 mg) was prepared similarly to Int. 2C18 from Ints. 1AC4/2C58 (0.21 g/0.18 g). This material was stirred 0.5 h in DCM/TFA (4:1, 2.5 mL), concentrated, and purified by SCX to give Int. 2O3 4-(1-((4-bromo-1-methyl-1H-pyrrolo[2,3-b]pyridin-2-yl)-methyl)piperidin-4-yl)-N-methyl-N-(4-(piperidin-4-ylmethyl)phenyl)-benzamide (0.20 g). This material (0.10 g), K₂CO₃ (90 mg), and 1-fluoro-4-nitrobenzene (22 mg) were stirred ON in DMF (2 mL) at 100° C. The OL (EtOAc/water) was washed with brine, dried, concentrated, and purified by FC (heptane/EtOAc 1:1 to 0:1) to give Int. 2O4 4-(1-((4-bromo-1-methyl-1H-pyrrolo[2,3-b]pyridin-2-yl)methyl)-piperidin-4-yl)-N-methyl-N-(4-((1-(4-nitrophenyl)-piperidin-4-yl)methyl)phenyl)benzamide (98 mg).

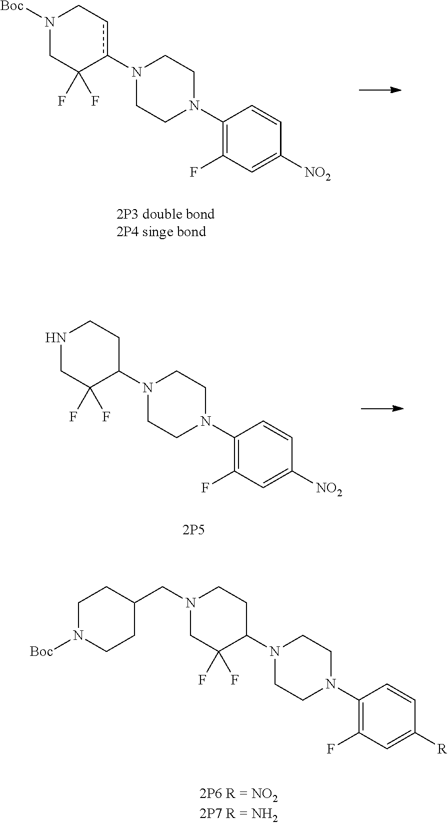

NaOAc (2.7 g) and 1-(2-fluoro-4-nitrophenyl)piperazine (3.5 g, HCl salt) were stirred 0.5 h in toluene/CAN/AcOH (8.8:3.8:1, 54 mL). tert-Butyl 3,3-difluoro-4-oxopiperidine-1-carboxylate (4.4 g) was added and stirring continued ON at 100° C. The filtrate after filtration was concentrated to give Int. 2P3 tert-butyl 3,3-difluoro-4-(4-(2-fluoro-4-nitro-phenyl)piperazin-1-yl)-3,6-dihydropyridine-1 (2H)-carboxylate (7 g).

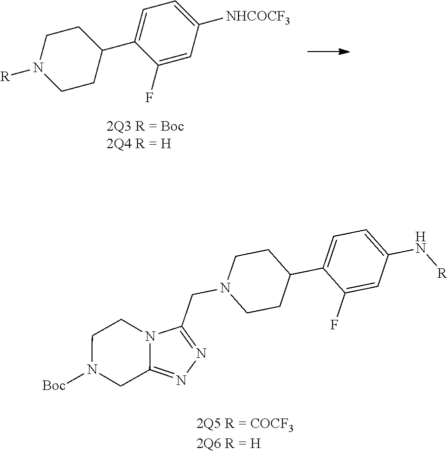

TFAA (2.5 mL) was added to a solution of tert-butyl 4-(4-amino-2-fluorophenyl)-piperidine-1-carboxylate (3.5 g) in toluene/Et₃N (6:1, 35 mL) at 0° C. before stirring ON at 0° C. to RT. The OL (EtOAc/water) was dried and concentrated to give Int. 2Q3 tert-butyl 4-(2-fluoro-4-(2,2,2-trifluoro-acetamido)phenyl)piperidine-1-carboxylate (4.5 g). This material was stirred 2 h in 2.4M HCl in dioxane (50 mL) at 0° C. to RT and concentrated to give Int. 2Q4 2,2,2-trifluoro-N-(3-fluoro-4-(piperidin-4-yl)-phenyl)-acetamide (3.3 g, HCl salt). Ints. 2Q4/2Q8 (0.65 g, HCl salt/0.79 g) were stirred ON in DMF/DIPEA (3:1, 8 mL). The OL (EtOAc/water) was washed with brine, dried, concentrated, and purified by FC (DCM/MeOH 95:5 to 9:1) to give Int. 2Q5 tert-butyl 3-((4-(2-fluoro-4-(2,2,2-trifluoro-acetamido)phenyl)piperidin-1-yl)methyl)-5,6-dihydro-[1,2,4]triazolo[4,3-a]pyrazine-7 (8H)-carboxylate (0.45 g). This material and K₂CO₃ (1.2 g) were stirred ON in MeOH/water (1:1, 8 mL). The OL (EtOAc/water) was washed with brine, dried, and concentrated to give Int. 2Q6 tert-butyl 3-((4-(4-amino-2-fluoro-phenyl)piperidin-1-yl)-methyl)-5,6-dihydro-[1,2,4]triazolo[4,3-a]pyrazine-7 (8H)-carboxylate (0.29 g).

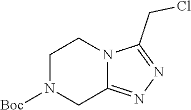 [SMILES: CC(C)(C)OC(=O)N1CCn2c(CCl)nnc2C1]

tert-Butyl 3-(hydroxymethyl)-5,6-dihydro-[1,2,4]-triazolo[4,3-a]pyrazine-7 (8H)-carboxylate (0.70 g) was stirred ON in DCM (10 mL), MsCl (0.4 mL), and Et₃N (1.5 mL) at 0° C. to RT. The OL (DCM/water) was dried and concentrated to give Int. 2Q8 tert-butyl 3-(chloromethyl)-5,6-dihydro-[1,2,4]triazolo[4,3-a]pyrazine-7 (8H)-carboxylate (0.81 g).

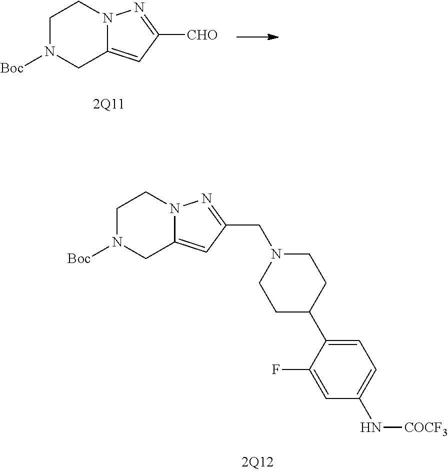

DMP (1.5 g) and tert-butyl 2-(hydroxymethyl)-6,7-dihydropyrazolo[1,5-a]pyrazine-5 (4H)-carboxylate (0.45 g) were stirred 3 h in DCM (4 mL) at 0° C. to RT. The OL (DCM/aq. NaHCO₃) was washed with brine, dried, concentrated, and purified by FC (pentane/EtOAc 3:2 to 1:1) to give Int. 2Q11 tert-butyl 2-formyl-6,7-dihydro-pyrazolo-[1,5-a]pyrazine-5 (4H)-carboxylate (0.22 g). Ints. 2Q11/2Q4 (0.2 g/0.26 g, HCl salt) were stirred 4 h in DCE/DIPEA (10:1, 2.2 mL). STAB (0.34 g) was added before stirring ON at 0° C. to RT. The OL (DCM/water) was washed with brine, dried, concentrated, and purified by FC (DCM/MeOH 98:2 to 95:5) to give Int. 2Q12 tert-butyl 2-((4-(2-fluoro-4-(2,2,2-trifluoro-acetamido)phenyl)-piperidin-1-yl)methyl)-6,7-dihydro-pyrazolo[1,5-a]pyrazine-5 (4H)-carboxylate (0.17 g).

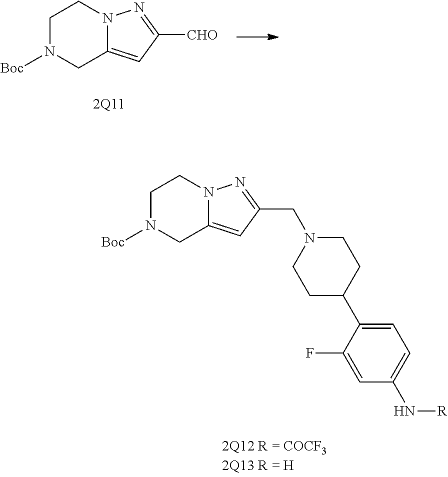

DMP (1.5 g) and tert-butyl 2-(hydroxymethyl)-6,7-dihydropyrazolo[1,5-a]pyrazine-5 (4H)-carboxylate (0.45 g) were stirred 3 h in DCM (4 mL) at 0° C. to RT. The OL (DCM/aq. NaHCO₃) was washed with brine, dried, concentrated, and purified by FC (pentane/EtOAc 3:2 to 1:1) to give Int. 2Q11 tert-butyl 2-formyl-6,7-dihydro-pyrazolo-[1,5-a]pyrazine-5 (4H)-carboxylate (0.22 g). Ints. 2Q11/2Q4 (0.2 g/0.26 g, HCl salt) were stirred 4 h in DCE/DIPEA (10:1, 2.2 mL). STAB (0.34 g) was added before stirring ON at 0° C. to RT. The OL (DCM/water) was washed with brine, dried, concentrated, and purified by FC (DCM/MeOH 98:2 to 95:5) to give Int. 2Q12 tert-butyl 2-((4-(2-fluoro-4-(2,2,2-trifluoro-acetamido)phenyl)-piperidin-1-yl)methyl)-6,7-dihydro-pyrazolo[1,5-a]pyrazine-5 (4H)-carboxylate (0.17 g).

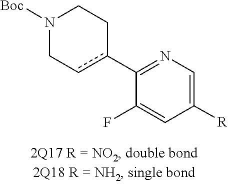

NaHCO₃ (1.4 g), 2-chloro-3-fluoro-5-nitro-pyridine (1.0 g), tert-butyl 4-(4,4,5,5-tetramethyl-1,3,2-dioxaborolan-2-yl)-3,6-dihydropyridine-1 (2H)-carboxylate (1.93 g), and PdDPPFCl₂-DCM (0.37 g) were degassed in dioxane/water (5:1, 12 mL) before stirring ON at 100° C. and filtered. The OL (EtOAc/water) was dried, concentrated, and purified by FC (pentane/EtOAc 1:0 to 7:3) to give Int. 2Q17 tert-butyl 3-fluoro-5-nitro-3′,6′-dihydro-[2,4′-bipyridine]-1′(2′H)-carboxylate (0.6 g). Int. 2Q17 (7.0 g) and 10% Pd/C (2 g) were stirred 8 h in THF/EtOAc (1:1, 80 mL) under a H₂ atmosphere (60 psi), filtered, and concentrated to give Int. 2Q18 tert-butyl 4-(5-amino-3-fluoropyridin-2-yl)piperidine-1-carboxylate (6 g).

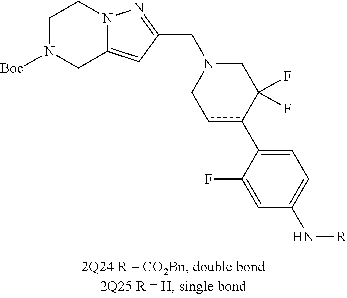

Ints. 2Q11/2M9 (1.2 g/2.0 g) were stirred 3 h in DCE/DIPEA (5:1, 24 mL) at 0° C. to RT. STAB (3.1 g) was added at 0° C. before stirring ON at 0° C. to RT. The OL (DCM/MeOH (9:1)/water) was washed with brine, dried, concentrated, and purified by FC (pentane/EtOAc 9:1 to 2:3) to give Int. 2Q24 tert-butyl 2-((4-(4-(((benzyloxy)carbo-nyl)amino)-2-fluorophenyl)-3,3-difluoro-3,6-dihydro-pyridin-1 (2H)-yl)methyl)-6,7-dihydropyrazolo[1,5-a]-pyrazine-5 (4H)-carboxylate (1.5 g).

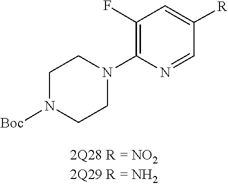

2-Chloro-3-fluoro-5-nitropyridine (4.0 g), tert-butyl piperazine-1-carboxylate (4.2 g), and K₂CO₃ (9.4 g) were stirred ON in DMF (7 mL) at 85° C. The OL (EtOAc/water) was washed with brine, dried, concentrated, and triturated in pentane and dried to give Int. 2Q28 tert-butyl 4-(3-fluoro-5-nitropyridin-2-yl)-piperazine-1-carboxylate (7 g). Int. 2Q28 (1.7 g) was hydrogenated ON using 10% Pd/C (0.5 g) in dioxane/THF (8 mL), filtered, concentrated, and triturated in pentane to give Int. 2Q29 tert-butyl 4-(5-amino-3-fluoropyridin-2-yl)piperazine-1-carboxylate (1.4 g).

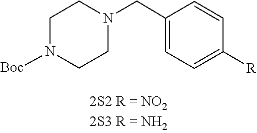

1-(Chloromethyl)-4-nitrobenzene (0.56 g), tert-butyl piperazine-1-carboxylate (0.50 g), and K₂CO₃ (0.75 g) were stirred 2 h in DMF (5 mL) at 70° C. The OL (EtOAc/water) was washed with brine, dried, and concentrated. The residue was purified by FC (heptane/EtOAc 9:1 to 0:1) to give Int. 2S2 tert-butyl 4-(4-nitro-benzyl)piperazine-1-carboxylate (0.83 g). Int. 2S2 (0.2 g), zinc powder (0.19 g), and NH₄Cl (0.15 g) were stirred 1 h in THF/water (1:1, 12 mL) and filtered. The OL (EtOAc/water) was washed with brine, dried, concentrated, and purified by FC (heptane/EtOAc 4:1 to 0:1) to give Int. 2S3 tert-butyl 4-(4-aminobenzyl)-piperazine-1-carboxylate (70 mg).

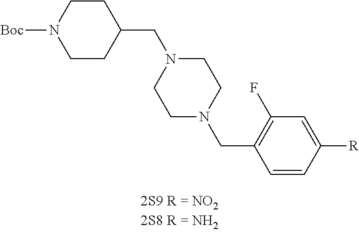

2-Fluoro-4-nitrobenzaldehyde (0.10 g), Int. 3E5 (0.25 g), and STAB (0.38 g) were stirred ON in DCE/DIPEA (19:1, 4.2 mL). STAB (0.10 g) and Int. 3E5 (40 mg) were added and stirring continued 2 h. The mixture was concentrated and purified by FC (heptane/EtOAc 1:1 to 0:1) to give Int. 2S9 tert-butyl 4-[[4-[(2-fluoro-4-nitro-phenyl)methyl]piperazin-1-yl]methyl]piperidine-1-carboxylate (0.18 g). This material, NH₄Cl (45 mg), and iron powder (0.16 g) were stirred 1.5 h in EtOH/water (4:1, 10 mL) at 80° C. The OL (EtOAc/water) was washed with brine, dried, concentrated, and purified by FC to give Int. 2S8 tert-butyl 4-[[4-[(4-amino-2-fluoro-phenyl)methyl]piperazin-1-yl]methyl]piperidine-1-carboxylate (0.15 g).

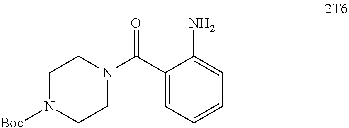 [SMILES: CC(C)(C)OC(=O)N1CCN(C(=O)c2ccccc2N)CC1]

2H-Benzo[d][1,3]oxazine-2,4 (1H)-dione (1.0 g) and tert-butyl piperazine-1-carboxylate (1.1 g) were stirred 2 h in dioxane (9 mL) at 90° C. The OL (EtOAc/water) was washed with sat. aq. NH₄Cl and brine, dried, and concentrated, and purified by FC to give Int. 2T6 tert-butyl 4-(2-amino-benzoyl)-piperazine-1-carboxylate (1.8 g).

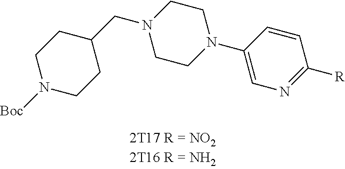

5-Fluoro-2-nitropyridine (0.13 g), Int. 3E5 (0.27 g), K₂CO₃ (0.30 g) were stirred ON in DMF (4 mL) at 60° C. The OL (water/EtOAc) was washed with brine, dried, concentrated, and purified by FC (heptane to EtOAc) to give Int. 2T17 tert-butyl 4-((4-(6-nitropyridin-3-yl)piperazin-1-yl)methyl)piperidine-1-carboxylate (0.26 g). Int. 2T17 (0.25 g) was hydrogenated ON using 10% Pd/C (80 mg) in MeOH/EtOAc (2:1, 15 mL), filtered, concentrated, and purified by FC (heptane/EtOAc/DCM/MeOH 1:0:0:0 to 0:9:0.5:0.5) to give Int. 2T16 tert-butyl 4-((4-(6-aminopyridin-3-yl)piperazin-1-yl)methyl)piperidine-1-carboxylate (0.22 g).

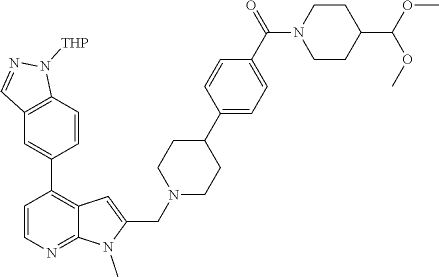

Int. 3A2 (0.50 g, HCl salt) and HATU (0.49 g) were stirred 0.3 h in DMF/DIPEA (20:1, 10.5 mL). 4-(Dimethoxy-methyl)piperidine (0.16 g) was added and stirring continued ON. The OL (water/EtOAc) was washed with brine, dried, concentrated, and purified by FC (DCM/MeOH 1:0 to 95:5) to give Int. 3D4 (4-(dimethoxy-methyl)-piperidin-1-yl)(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)phenyl)methanone (0.20 g).

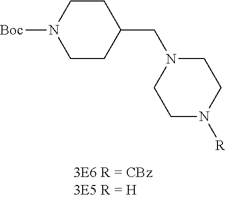

tert-Butyl 4-formylpiperidine-1-carboxylate (25 g) and benzyl piperazine-1-carboxylate (28.4 g) were stirred 4 h in DCE/DIPEA (4.1:1, 311 mL). STAB (49.5 g) was added and stirring continued ON at 0° C. to RT ON. The OL (water/DCM) was washed with brine, dried, concentrated, and purified by FC (pentane/EtOAc 1:1) to give Int. 3E6 benzyl piperazine-1-carboxylate (70 g). This material was hydrogenated ON using 10% Pd/C (13 g) in EtOH (1 L), filtered, and concentrated to give Int. 3E5 tert-butyl 4-(piperazin-1-ylmethyl)piperidine-1-carboxylate (40 g).

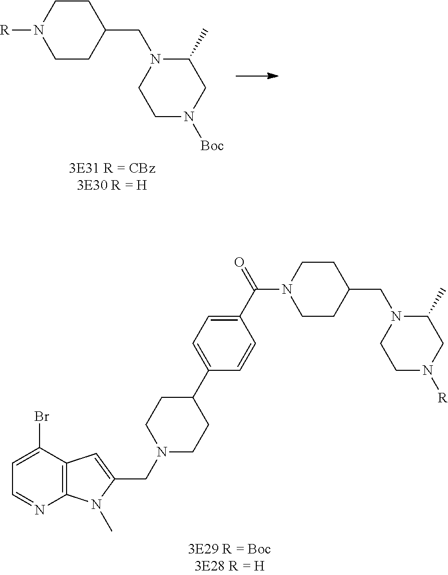

Benzyl 4-formylpiperidine-1-carboxylate (0.50 g) and tert-butyl (R)-3-methylpiperazine-1-carboxylate (0.40 g) were stirred 3 h in DCE/DIPEA (7:1, 5.7 mL). STAB (0.85 g) was added and stirring continued 3 h at 0° C. to RT. The OL (water/DCM) was dried, concentrated, and purified by FC (hexane/EtOAc 1:4) to give Int. 3E31 tert-butyl (R)-4-[(1-benzyloxycarbonyl-4-piperidyl)-methyl]-3-methyl-piperazine-1-carboxylate (0.65 g). This material was hydrogenated 8 h using and 5% Pd/C (0.1 g) in MeOH (5 mL). Filtration and concentration gave Int. 3E30 tert-butyl (3R)-3-methyl-4-(4-piperidylmethyl)-piperazine-1-carboxylate (0.35 g). Ints. 3E30/1AC4 (58 mg/70 mg) and HATU (87 mg) were stirred 3 h in DMF/DIPEA (28.1:1, 1.66 mL). The OL (EtOAc/water) was washed with brine, dried, concentrated, and HPLC-purified to give Int. 3E29 tert-butyl (R)-4-((1-(4-(1-((4-bromo-1-methyl-1H-pyrrolo[2,3-b]pyridin-2-yl)-methyl)piperidin-4-yl)benzoyl)piperidin-4-yl)methyl)-3-methyl-piperazine-1-carboxylate (60 mg). This material was stirred 0.5 h in DCM/TFA (2:1; 3 mL). The residue after concentration was SCX-purified to give Int. 3E28 (R)-(4-(1-((4-bromo-1-methyl-1H-pyrrolo[2,3-b]pyridin-2-yl)methyl)piperidin-4-yl)phenyl)(4-((2-methyl-piperazin-1-yl)methyl)-piperidin-1-yl)methanone (40 mg).

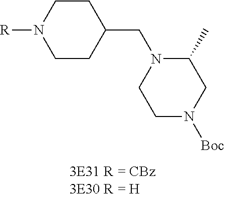

Benzyl 4-formylpiperidine-1-carboxylate (0.50 g) and tert-butyl (R)-3-methylpiperazine-1-carboxylate (0.40 g) were stirred 3 h in DCE/DIPEA (7:1, 5.7 mL). STAB (0.85 g) was added and stirring continued 3 h at 0° C. to RT. The OL (water/DCM) was dried, concentrated, and purified by FC (hexane/EtOAc 1:4) to give Int. 3E31 tert-butyl (R)-4-[(1-benzyloxycarbonyl-4-piperidyl)-methyl]-3-methyl-piperazine-1-carboxylate (0.65 g). This material was hydrogenated 8 h using and 5% Pd/C (0.1 g) in MeOH (5 mL). Filtration and concentration gave Int. 3E30 tert-butyl (3R)-3-methyl-4-(4-piperidylmethyl)-piperazine-1-carboxylate (0.35 g).

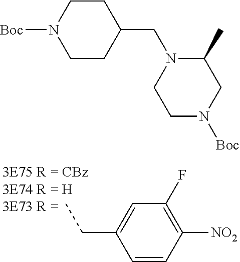

Benzyl (S)-3-methylpiperazine-1-carboxylate (2.0 g) and tert-butyl 4-formylpiperidine-1-carboxylate (1.8 g) were stirred 3 h in DCE (30 mL). STAB (3.6 g) was added and stirring continued ON. The OL (water/DCM) was dried, concentrated, and purified by FC (pentane/EtOAc 1:0 to 4:1) to give Int. 3E75 benzyl (3S)-4-[(1-tert-butoxy-carbonyl-4-piperidyl)methyl]-3-methyl-piperazine-1-carboxylate (3.0 g). This material was hydrogenated ON using 10% Pd/C (0.45 g) in MeOH (40 mL), filtered, and concentrated to give Int. 3E74 tert-butyl 4-[[(2S)-2-methylpiperazin-1-yl]methyl]piperidine-1-carboxylate (1.9 g). Int. 3E74 (5.0 g) and 3-fluoro-4-nitrobenzaldehyde (3.0 g) were stirred 3 h in DCE/DIPEA (5.8:1, 59 mL). STAB (7.1 g) was added at 0° C. and stirring continued ON at 0° C. to RT. The OL (water/DCM) was dried, concentrated, and purified by FC (pentane/EtOAc 1:0 to 3:2) to give Int. 3E73 tert-butyl (S)-4-((4-(3-fluoro-4-nitrobenzyl)-2-methylpiperazin-1-yl)methyl)piperidine-1-carboxylate (1.8 g).

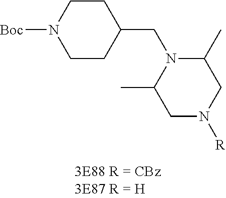

Benzyl 3,5-cis-dimethylpiperazine-1-carboxylate (1.5 g) and tert-butyl 4-formylpiperidine-1-carboxylate (1.7 g) were stirred ON in DCE/DIPEA (8.6:1, 6.7 mL) at 0° C. to RT. STAB (4.5 g) was added and stirring continued ON at 0° C. to RT. The OL (water/DCM) was washed with brine, dried and, concentrated. The residue was purified by FC (pentane/EtOAc 7:3 to 2:3) to give Int. 3E88 benzyl 4-((1-(tert-butoxycarbonyl)-piperidin-4-yl)-methyl)-3,5-cis-dimethylpiperazine-1-carboxylate (1.75 g). 1.7 g of this material was hydrogenated ON using 10% Pd/C (0.5 g) in EtOAc (10 mL), filtered, and concentrated to give Int. 3E87 tert-butyl 4-[[2,6-cis-dimethyl-piperazin-1-yl]methyl]-piperidine-1-carboxylate (1.1 g).

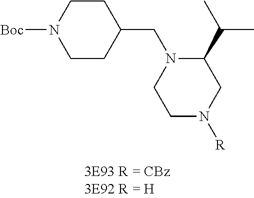

Benzyl (S)-3-iso-propylpiperazine-1-carboxylate (2.0 g, HCl salt) and tert-butyl 4-formylpiperidine-1-carboxylate (1.6 g) were stirred 4 h in DCE/DIPEA (4.5:1, 24.4 mL). STAB (2.8 g) was added and stirring continued 4 h. The OL (water/DCM) was washed with brine, dried, concentrated, and purified by FC (pentane/EtOAc 1:0 to 4:1) to give Int. 3E93 benzyl (3S)-4-[(1-tert-butoxycarbonyl-4-piperidyl)methyl]-3-iso-propyl-piperazine-1-carboxylate (1.9 g). 1.0 g of this material was hydrogenated ON using 10% Pd/C (0.2 g) in THF/EtOAc (1:1; 20 mL), filtered, and concentrated to give Int. 3E92 tert-butyl 4-[[(2S)-2-iso-propylpiperazin-1-yl]methyl]piperidine-1-carboxylate (0.6 g).

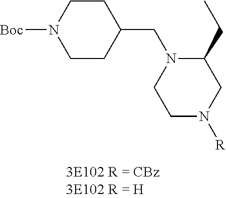

Benzyl (3S)-3-ethylpiperazine-1-carboxylate (2.66 g) and Int. and tert-butyl 4-formylpiperidine-1-carboxylate (2.51 g) were stirred 4 h in DCE/DIPEA (2.2:1, 29.3 mL). STAB (4.5 g) was added and stirring continued ON. The OL (water/DCM) was washed with brine, dried and, concentrated to give Int. 3E102 benzyl (3S)-4-[(1-tert-butoxy-carbonyl-4-piperidyl)methyl]-3-ethyl-piperazine-1-carboxylate (3.4 g). 0.73 g of this material and 10% Pd/C (0.18 g) were hydrogenated 4 h in THF/EtOAc (1:2.3, 20 mL), filtered, and concentrated to give Int. 3E101 tert-butyl 4-[[(2S)-2-ethylpiperazin-1-yl]methyl]piperidine-1-carboxylate (0.5 g).

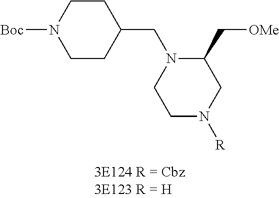

Benzyl (3R)-3-(methoxymethyl)piperazine-1-carboxylate (90 mg, HCl salt) and tert-butyl 4-formyl-piperidine-1-carboxylate (70 mg) were stirred 4 h in DCE/DIPEA (11.5:1, 3.3 mL). STAB (127 mg) was added and stirring continued ON. The OL (water/DCM) was washed with brine, dried and, concentrated. The residue was purified by FC (DCM/MeOH 95:5 to 9:1) to give Int. 3E124 benzyl (3R)-4-[(1-tert-butoxy-carbonyl-4-piperidyl)-methyl]-3-(methoxymethyl)piperazine-1-carboxylate (50 mg). Int. 3E124 (0.6 g) was hydrogenated 4 h using 10% Pd/C (0.35 g) in THF/EtOAc (1:1; 40 mL), filtered, concentrated, and triturated in pentane to give Int. 3E123 tert-butyl 4-[[(2R)-2-(methoxymethyl)piperazin-1-yl]methyl]piperidine-1-carboxylate (0.5 g).

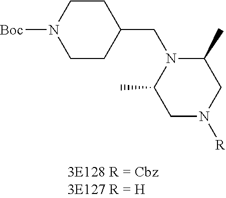

Benzyl (3S,5S)-3,5-dimethylpiperazine-1-carboxylate (1.2 g, HCl salt) and tert-butyl 4-formyl-piperidine-1-carboxylate (1.1 g) were stirred 4 h in DCE/DIPEA (1:1.9, 5.7 mL). STAB (1.8 g) was added at 0° C. and stirring continued ON at 0° C. to RT. The OL (water/DCM) was washed with brine, dried and, concentrated. The residue was purified by FC (pentane/EtOAc 7:3) to give Int. 3E128 benzyl (3S,5S)-4-[(1-tert-butoxy-carbonyl-4-piperidyl)-methyl]-3,5-dimethyl-piperazine-1-carboxylate (1.1 g). This material was hydrogenated 4 h using 10% Pd/C (0.3 g) in MeOH (35 mL), filtered, concentrated, and triturated in MeOH to give Int. 3E127 tert-butyl 4-[[(2S,6S)-2,6-dimethylpiperazin-1-yl]-methyl]piperidine-1-carboxylate (0.51 g).

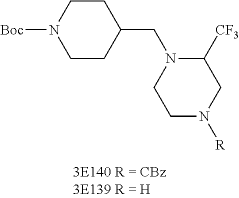

Benzyl 3-(trifluoromethyl)piperazine-1-carboxylate hydrochloride (2.4 g) and tert-butyl 4-formyl-piperidine-1-carboxylate (1.4 g) were stirred 4 h in DCE/DIPEA (6.4:1, 29 mL). STAB (3.1 g) was added at 0° C. and stirring continued ON at 0° C. to RT. The OL (water/DCM) was washed with brine, dried and, concentrated to give Int. 3E140 benzyl 4-((1-(tert-butoxycarbonyl)-piperidin-4-yl)methyl)-3-(trifluoro-methyl)piperazine-1-carboxylate (1.6 g). This material was hydrogenated ON using 10% Pd/C (0.3 g) in EtOAc/water (7:3; 10 mL), filtered, and concentrated to give Int. 3E139 tert-butyl 4-((2-(trifluoromethyl)-piperazin-1-yl)methyl)-piperidine-1-carboxylate (1.1 g).

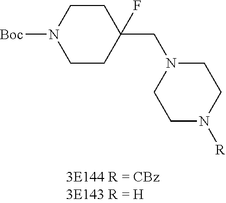

Benzyl piperazine-1-carboxylate (4.3 g) and tert-butyl 4-fluoro-4-formyl-piperidine-1-carboxylate (3.5 g) were stirred 3 h in DCE/DIPEA (3.6:1, 51 mL)). STAB (6.4 g) was added and stirring continued ON at 0° C. to RT. The OL (water/DCM) was washed with brine, dried, concentrated, and purified by FC (pentane/EtOAc 1:0 to 4:1) to give Int. 3E144 benzyl 4-((1-(tert-butoxy-carbonyl)-4-fluoropiperidin-4-yl)methyl)piperazine-1-carboxylate (4.0 g). This material was hydrogenated ON using 10% Pd/C (1.5 g) in THF/EtOAc (1:1; 30 mL), filtered, a concentrated to give Int. 3E143 tert-butyl 4-fluoro-4-(piperazin-1-ylmethyl)piperidine-1-carboxylate (2.3 g).

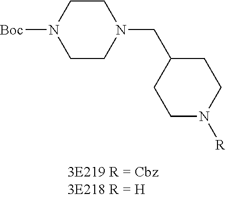

Benzyl 4-formylpiperidine-1-carboxylate (0.10 g) and tert-butyl piperazine-1-carboxylate (90 mg) were stirred 3 h in DCE/DIPEA (17.9:1, 5.3 mL) at 0° C. to RT. STAB (0.17 g) was added and stirring continued ON at 0° C. to RT. The OL (water/EtOAc) was dried and concentrated to give Int. 3E219 tert-butyl 4-[(1-benzyloxycarbonyl-4-piperidyl)-methyl]piperazine-1-carboxylate (0.10 g). Int. 3E219 (5.4 g) was hydrogenated ON using 10% Pd/C in EtOH (60 mL), filtered, and concentrated to give Int. 3E218 tert-butyl 4-(piperidin-4-ylmethyl)piperazine-1-carboxylate (3.4 g).

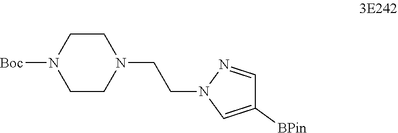

tert-Butyl 4-(2-hydroxyethyl)piperidine-1-carboxylate (0.26 g), 4-(4,4,5,5-tetramethyl-1,3,2-dioxaborolan-2-yl)-1H-pyrazole (0.20 g), and Ph₃P (0.3 g) were dissolved in THF (5 mL) at 0° C. DIAD (0.22 mL) was added at 3-6° C. and stirring was continued 2 h at RT. The residue after concentration was purified by FC (heptane/EtOAc 1:1 and DCM/EtAOAc 0:1 to 2:3) to give Int. 3E242 tert-butyl 4-(2-(4-(4,4,5,5-tetramethyl-1,3,2-dioxaborolan-2-yl)-1H-pyrazol-1-yl)ethyl)piperidine-1-carboxylate (0.11 g).

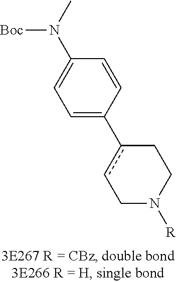

Benzyl 4-(4,4,5,5-tetramethyl-1,3,2-dioxaborolan-2-yl)-3,6-dihydropyridine-1 (2H)-carboxylate (7.1 g), tert-butyl (4-bromophenyl)(methyl)carbamate (5.0 g), NaHCO₃ (4.3 g), and PdDPPFCl₂-DCM (0.7 g) were degassed in dioxane/water (9:1; 50 mL) and stirred ON at 100° C. and filtered. The OL (water/EtOAc) was washed with brine, dried, concentrated, and purified by FC (pentane/EtOAc 9:1 to 88:12) to give Int. 3E267 benzyl 4-(4-((tert-butoxy-carbonyl)(methyl)amino)phenyl)-3,6-dihydropyridine-1 (2H)-carboxylate (4.5 g). 1.0 g of this material was hydrogenated ON using 10% Pd/C (0.5 g) in MeOH (12 mL), filtered, and concentrated to give Int. 3E266 tert-butyl methyl(4-(piperidin-4-yl)phenyl)carbamate (0.6 g).

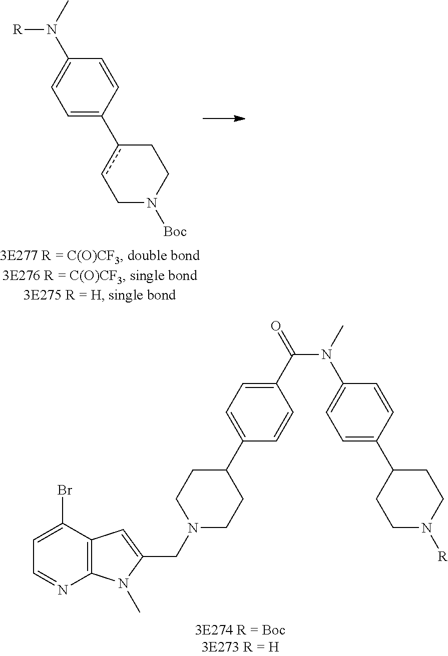

N-(4-bromophenyl)-2,2,2-trifluoro-N-methylacetamide (3.5 g), PdDPPFCl₂-DCM (0.44 g), tert-butyl 4-(4,4,5,5-tetramethyl-1,3,2-dioxaborolan-2-yl)-3,6-dihydropyridine-1 (2H)-carboxylate (3.3 g), and NaHCO₃ (1.8 g) were degassed in dioxane/water (10:1, 34 mL) and stirred ON at 100° C. and filtered. The OL (EtOAc/water) was washed with brine, dried, and concentrated. The residue was mixed with two additional batches prepared similarly and purified by FC (pentane/EtOAc 9:1 to 85:15) to give Int. 3E277 tert-butyl 4-(4-(2,2,2-trifluoro-N-methylacetamido)phenyl)-3,6-dihydropyridine-1 (2H)-carboxylate (8.3 g). 3.0 g of this material was hydrogenated ON using 10% Pd/C in MeOH (30 mL), filtered, concentrated, and purified by FC (pentane/EtOAc 9:1 to 85:15) to give Int. 3E276 tert-butyl 4-(4-(2,2,2-trifluoro-N-methylacetamido)phenyl)piperidine-1-carbo-xylate (2.5 g). 0.8 g of this material and K₂CO₃ (0.7 g) were stirred 3 h in MeOH (7 mL) and concentrated. The OL (water/DCM) was dried and concentrated to give Int. 3E275 tert-butyl 4-(4-(methylamino)phenyl)-piperidine-1-carboxylate (0.5 g). Ints. 1AC4/3E275 (0.5 g/0.58 g) and HATU (0.67 g) were stirred ON in DMF/DIPEA (24.5:1, 15.6 mL) and diluted with water to precipitate Int. 3E274 tert-butyl 4-(4-(4-(1-((4-bromo-1-methyl-1H-pyrrolo-[2,3-b]pyridin-2-yl)methyl)piperidin-4-yl)-N-methyl-benzamido)-phenyl)piperidine-1-carboxylate (0.6 g). 0.18 g of this material was stirred 0.5 h in DCM/TFA (2:1; 1.5 mL), concentrated, and SCX-purified to give Int. 3E273 4-[1-[(4-bromo-1-methyl-pyrrolo[2,3-b]pyridin-2-yl)methyl]-4-piperidyl]-N-methyl-N-[4-(4-piperidyl)phenyl]-benzamide (0.15 g).

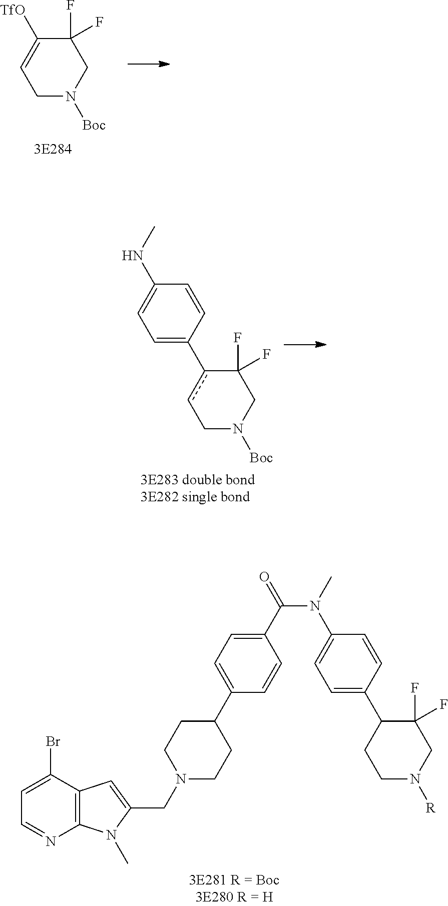

Tf₂O (1.6 g) was added to solution of tert-butyl 3,3-difluoro-4-oxopiperidine-1-carboxylate (0.88 g) in DCM/Et₃N (5:1, 9.6 mL) at 0° C. The mixture was stirred 0.5 h at 0° C., quenched with water, dried, and concentrated. The residue was combined with another batch prepared similarly on 4 g scale and purified by FC (pentane/EtOAc 9:1 to 4:1) to give Int. 3E284 tert-butyl 3,3-difluoro-4-(trifluoromethyl-sulfonyloxy)-2,6-dihydro-pyridine-1-carbo-xylate (1.5 g). 0.74 g of this material, N-methyl-4-(4,4,5,5-tetramethyl-1,3,2-dioxaborolan-2-yl)aniline (0.59 g), Na₂CO₃ (0.64 g), and PdDPPFCl₂-DCM (0.19 g) were degassed in dioxane/water (4:1, 15 mL) and stirred ON at 55° C. and filtered. The OL (EtOAc/water) dried and concentrated. The residue was purified by FC (pentane/EtOAc 7:3 to 3:2) and by HPLC to give Int. 3E283 tert-butyl 3,3-difluoro-4-[4-(methylamino)phenyl]-2,6-dihydro-pyridine-1-carboxylate (0.4 g). This material was hydrogenated ON using 10% Pd/C (0.15 g) in EtOAc/THF (1:1, 6 mL), filtered, and concentrated to give Int. 3E282 tert-butyl 3,3-difluoro-4-[4-(methylamino)-phenyl]-piperidine-1-carboxylate (0.34 g). Ints. 1AC4/3E282 (87 mg/60 mg) and HATU (84 mg) were stirred ON in DMF/DIPEA (6.3:1, 2.3 mL). HATU (70 mg) and DIPEA (0.16 mL) added and stirring continued ON at 50° C. HATU (70 mg) and DIPEA (0.16 mL) were added and stirring continued ON at 50° C. The OL (EtOAc/water) was concentrated and purified by FC (heptane/EtOAc 1:0 to 0:1) and by HPLC to give Int. 3E281 tert-butyl 4-[4-[[4-[1-[(4-bromo-1-methyl-pyrrolo[2,3-b]pyridin-2-yl)methyl]-4-piperidyl]benzoyl]-methyl-amino]phenyl]-3,3-difluoro-piperidine-1-carboxylate (82 mg). A solution of this material in DCM/TFA (1:1; 2 mL) was stirred 0.5 h, concentrated and SCX-purified to give Int. 3E280 4-[1-[(4-bromo-1-methyl-pyrrolo-[2,3-b]pyridin-2-yl)methyl]-4-piperidyl]-N-[4-(3,3-difluoro-4-piperidyl)phenyl]-N-methyl-benzamide (52 mg).

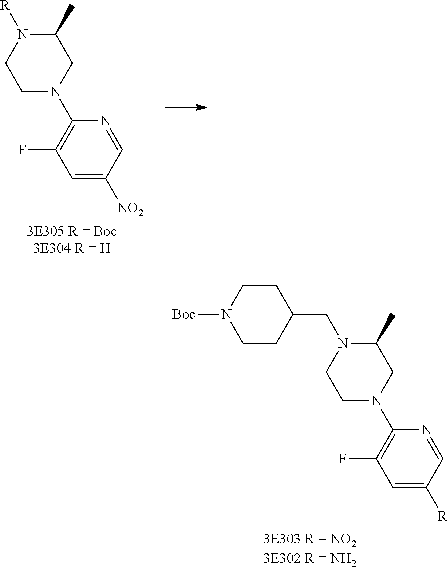

2-Chloro-3-fluoro-5-nitro-pyridine (0.50 g), tert-butyl (R)-2-methylpiperazine-1-carboxylate (0.62 g), and K₂CO₃ (1.6 g) were stirred ON in ACN (20 mL) at 85° C., concentrated, and triturated in water to give Int. 3E305 tert-butyl (2R)-4-(3-fluoro-5-nitro-2-pyridyl)-2-methyl-piperazine-1-carboxylate (0.84 g). This material was stirred 6 h in DCM/4M HCl in dioxane (1:2; 15 mL) at 0° C. and concentrated. The residue was triturated in Et₂O/pentane to give Int. 3E304 (3R)-1-(3-fluoro-5-nitro-2-pyridyl)-3-methyl-piperazine (0.65 g, HCl salt). This material and tert-butyl 4-formylpiperidine-1-carboxylate (0.42 g) were stirred ON in DCE/DIPEA (6.5:1, 17.3 mL). STAB (1.9 g) was added and stirring was continued 6 h at 0° C. to RT. The OL (water/DCM) was dried, concentrated, and triturated with Et₂O and dried to give Int. 3E303 tert-butyl 4-[[(2R)-4-(3-fluoro-5-nitro-2-pyridyl)-2-methyl-piperazin-1-yl]methyl]piperidine-1-carboxylate (0.85 g). This material was hydrogenated ON using 10% Pd/C (0.4 g) in THF/EtOAc (1:2; 15 mL), filtered, and concentrated to give Int. 3E302 tert-butyl 4-[[(2R)-4-(5-amino-3-fluoro-2-pyridyl)-2-methyl-piperazin-1-yl]methyl]piperidine-1-carboxylate (0.65 g).

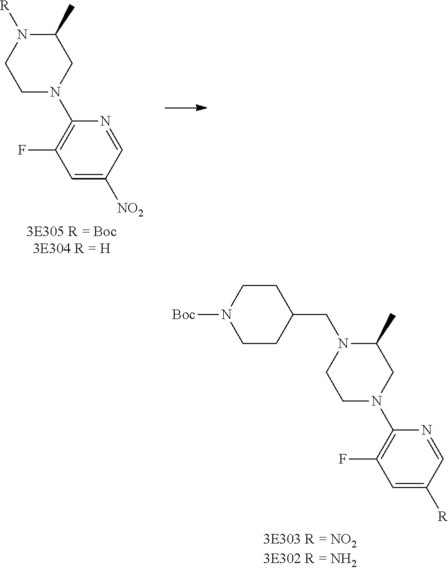

2-Chloro-3-fluoro-5-nitro-pyridine (0.50 g), tert-butyl (R)-2-methylpiperazine-1-carboxylate (0.62 g), and K₂CO₃ (1.6 g) were stirred ON in ACN (20 mL) at 85° C., concentrated, and triturated in water to give Int. 3E305 tert-butyl (2R)-4-(3-fluoro-5-nitro-2-pyridyl)-2-methyl-piperazine-1-carboxylate (0.84 g). This material was stirred 6 h in DCM/4M HCl in dioxane (1:2; 15 mL) at 0° C. and concentrated. The residue was triturated in Et₂O/pentane to give Int. 3E304 (3R)-1-(3-fluoro-5-nitro-2-pyridyl)-3-methyl-piperazine (0.65 g, HCl salt). This material and tert-butyl 4-formylpiperidine-1-carboxylate (0.42 g) were stirred ON in DCE/DIPEA (6.5:1, 17.3 mL). STAB (1.9 g) was added and stirring was continued 6 h at 0° C. to RT. The OL (water/DCM) was dried, concentrated, and triturated with Et₂O and dried to give Int. 3E303 tert-butyl 4-[[(2R)-4-(3-fluoro-5-nitro-2-pyridyl)-2-methyl-piperazin-1-yl]methyl]piperidine-1-carboxylate (0.85 g). This material was hydrogenated ON using 10% Pd/C (0.4 g) in THF/EtOAc (1:2; 15 mL), filtered, and concentrated to give Int. 3E302 tert-butyl 4-[[(2R)-4-(5-amino-3-fluoro-2-pyridyl)-2-methyl-piperazin-1-yl]methyl]piperidine-1-carboxylate (0.65 g).

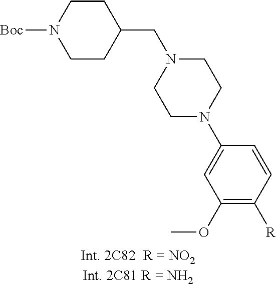

4-Fluoro-2-methoxy-1-nitro-benzene (12.5 g), K₂CO₃ (24 g), and tert-butyl 4-(piperazin-1-ylmethyl)-piperidine-1-carboxylate (10 g) were stirred 12 h in DMF (100 mL) at 80° C. The OL (water/EtOAc) was dried, concentrated, and purified by FC (DCM/MeOH 1:0 to 9:1) to give Int. 2C82 tert-butyl 4-((4-(3-methoxy-4-nitrophenyl)piperazin-1-yl)methyl)piperidine-1-carboxylate (20 g). Int. 2C82 (20 g) and 10% Pd/C (5 g) were stirred ON in MeOH (200 mL) at 70° C. under an atmosphere of hydrogen. The mixture was filtered and concentrated to give crude Int. 2C81 tert-butyl 4-((4-(4-amino-3-methoxyphenyl)piperazin-1-yl)methyl)-piperidine-1-carboxylate (15 g).

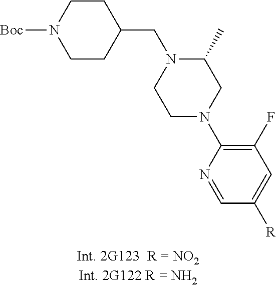

Int. 2F12 (18 g), 2-chloro-3-fluoro-5-nitropyridine (8 g), and K₂CO₃ (13 g) were stirred 16 h in ACN (60 mL) at 100° C. under MW conditions. The mixture was concentrated. The OL (water/EtOAc) was dried, concentrated, and purified by FC (PE/EtOAc 2:1) to give Int. 2G123 tert-butyl (R)-4-((4-(3-fluoro-5-nitro-pyridin-2-yl)-2-methylpiperazin-1-yl)methyl)piperidine-1-carboxylate (16 g). Int. 2G123 (16 g) and Pd/C 10% (5 g) were hydrogenated 16 h at 60 psi in MeOH (250 mL). The mixture was filtered and concentrated to give Int. 2G122 tert-butyl (R)-4-((4-(5-amino-3-fluoropyridin-2-yl)-2-methylpiperazin-1-yl)methyl)piperidine-1-carboxylate (13 g).

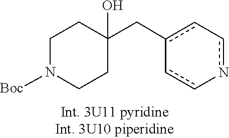

1.6M n-BuLi in hexane (200 mL) was added to a solution of 4-methylpyridine (26 mL) in THF (200 mL) at −78° C. and the mixture stirred for 30 min. tert-Butyl 4-oxopiperidine-1-carboxylate (59 g) in THF (100 mL) was added dropwise and stirring continued 0.5 h at −78° C. to RT. The OL (aq. NH₄Cl/EtOAc) was dried, concentrated, and purified by FC (PE/EtOAc 1:4 to 1:9) to give Int. 3U11 tert-butyl 4-hydroxy-4-(pyridin-4-ylmethyl)piperidine-1-carboxylate (30 g). Int. 3U11 (12 g) and PtO₂ (5.6 g) were hydrogenated at 100 psi for 16 h at 60° C. in EtOH (200 mL). The mixture was filtered and concentrated. The residue was stirred in aq. Na₂CO₃ and freeze-dried. The residue was dissolved in DCM, filtered, and concentrated to give Int. 3U10 tert-butyl 4-hydroxy-4-(piperidin-4-ylmethyl)-piperidine-1-carboxylate (10 g).

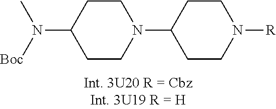

NaCNBH₃ (0.75 g), benzyl 4-oxopiperidine-1-carboxylate (2.2 g), and tert-butyl methyl(piperidin-4-yl)carbamate (2.0 g) were stirred 18 h in MeOH (30 mL). The mixture was concentrated. The OL (sat. aq. NaHCO₃/DCM) was dried, and concentrated to give Int. 3U20 benzyl 4-((tert-butoxy-carbonyl)(methyl)-amino)-[1,4′-bipiperidine]-1′-carboxylate (2.5 g). Int. 3U20 (2.5 g) and Pd/C (0.25 g) were hydrogenated 72 h at 735 psi in MeOH (30 mL) at 50° C. The mixture was filtered and concentrated to give Int. 3U19 tert-butyl[1,4′-bipiperidin]-4-yl(methyl)carbamate (1.8 g).

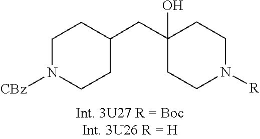

NaHCO₃ (19 g) and Int. 3U10 (10 g) were stirred 16 h in Et₂O/H₂O (1:1, 100 mL) and 50% benzyl chloro-formate in toluene (11 mL, added drop-wise) at 0° C. to RT. The OL (water/EtOAc) was dried, concentrated, and purified by FC (PE/EtOAc 4:1 to 1:1) to give Int. 3U27 tert-butyl 4-((1-((benzyloxy)-carbonyl)piperidin-4-yl)methyl)-4-hydroxypiperidine-1-carboxylate (8 g). Int. 3U27 (1 g) in HFP (15 mL) was heated under MW conditions for 16 h at 140° C. The mixture was combined with two other batches prepared similarly and concentrated to give Int. 3U26 benzyl 4-((4-hydroxypiperidin-4-yl)-methyl)piperidine-1-carboxylate (2.1 g).

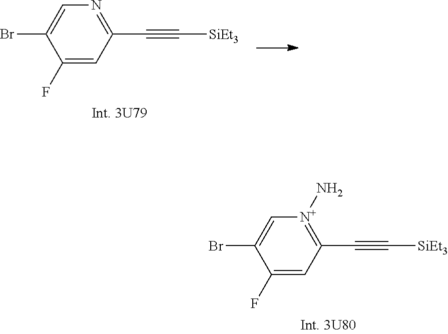

5-bromo-4-fluoro-2-((triethylsilyl)ethynyl)pyridine (11.6 g), Pd(PPh₃)₂Cl₂ (1.35 g), and CuI (0.1 g) were stirred ON in Et₃N (ACN (2.1/70 mL) triethyl(ethynyl)silane (7.4 mL) at 0° C. to RT under an inert atmosphere. The mixture was concentrated. The OL (water/EtOAc) was dried, concentrated, and purified by FC (hexane/DCM 1:0 to 0:1) to give Int. 3U79 5-bromo-4-fluoro-2-((triethylsilyl)-ethynyl)-pyridine (5.0 g). tert-butyl ((mesitylsulfonyl)oxy)carbamate (10.0 g) was added in portions to TFA (18 mL) at 0° C. and stirred for 1.5 h. The mixture was poured onto ice to precipitate a solid which was dissolved in DCM (80 mL). The OL (water/DCM) was dried and filtered and added slowly to a solution of Int. 3U79 (5.0 g) in DCM (80 mL) at 0° C. and stirred ON at RT and concentrated to give Int. 3U80 1-amino-5-bromo-4-fluoro-2-((triethylsilyl)ethynyl)pyridin-1-ium (8.0 g, 2,4,6-trimethylbenzenesulfonate salt).

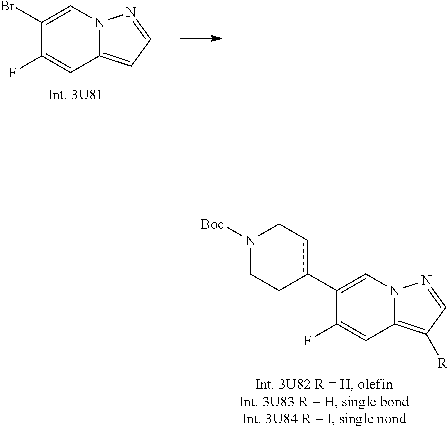

Ag₂CO₃ (5.0 g) and Int. 3U80 (8.0 g) were stirred 12 h in DMF (20 mL) at 0° C. to RT. The mixture was filtered. The OL (water/EtOAc) was dried, concentrated, and purified by FC (hexane/MTBE 1:0 to 0:1) to give Int. 3U81 6-bromo-5-fluoropyrazolo[1,5-a]pyridine (0.33 g). Int. 3U81 (0.33 g), tert-butyl 4-(4,4,5,5-tetramethyl-1,3,2-dioxaborolan-2-yl)-3,6-dihydropyridine-1 (2H)-carboxylate (0.57 g), Cs₂CO₃ (0.64 g), and PdDPPFCl₂-DCM (50 mg) were stirred ON in dioxane/H₂O (4:1, 5 mL) at 90° C. under an inert atmosphere. The mixture was concentrated. The OL (water/EtOAc) was dried, concentrated, and HPLC-purified to give Int. 3U82 tert-butyl 4-(5-fluoropyrazolo[1,5-a]pyridin-6-yl)-3,6-dihydro-pyridine-1 (2H)-carboxylate (0.31 g). Int. 3U82 (0.31 g) and Pd/C 5% wt. (0.1 g) were hydrogenated ON in MeOH (5 mL). The mixture was filtered and concentrated to give Int. 3U83 tert-butyl 4-(5-fluoropyrazolo[1,5-a]pyridin-6-yl)piperidine-1-carboxylate (0.27 g). NIS (0.19 g) and Int. 3U83 (0.27 g) were stirred 12 h in ACN (3 mL) at 0° C. to RT. The mixture was HPLC-purified to give Int. 3U84 tert-butyl 4-(5-fluoro-3-iodopyrazolo[1,5-a]pyridin-6-yl)piperidine-1-carboxylate (125 mg).

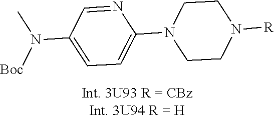

Pd₂(dba)₃ (2.3 g), Xantphos (2.8 g), Int. 1Y4 (23 g), benzyl piperazine-1-carboxylate (23 g), and NaO^{t}Bu (12 g) were stirred 16 h in toluene (200 mL) at 100° C. under an inert atmosphere. The mixture was filtered. The OL (water/EtOAc) was dried, concentrated, and purified by FC (PE/EtOAc 1:1 to 2:5) to give Int. 3U93 benzyl 4-(5-((tert-butoxycarbonyl)(methyl)amino)pyridin-2-yl)piperazine-1-carboxylate. Int. 3U93 (13 g) and Pd/C 10% wet (4 g) were hydrogenated 5 h at 60 psi in MeOH (150 mL). The mixture was filtered and concentrated to give Int. 3U94 tert-butyl methyl(6-(piperazin-1-yl)pyridin-3-yl)carbamate (8 g).

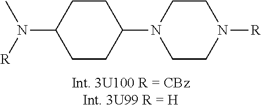

NaCNBH₃ (1.8 g), tert-butyl methyl(4-oxocyclohexyl)carbamate (5.0 g), benzyl piperazine-1-carboxylate (4.9 g) were stirred 18 h in MeOH (50 mL). The mixture was concentrated. The OL (sat. aq. NaHCO₃/DCM) was dried, and concentrated to give Int. 3U100 benzyl 4-(4-((tert-butoxy-carbonyl)(methyl)amino)cyclohexyl)-piperazine-1-carboxylate (6.1 g). Int. 3U100 (6.1 g) an Pd/C (0.61 g) were hydrogenated 72 h at 735 psi at 50° C. in MeOH (75 mL). The mixture was filtered and concentrated to give Int. 3U99 tert-butyl methyl(4-(piperazin-1-yl)cyclohexyl)carbamate (1.8 g).

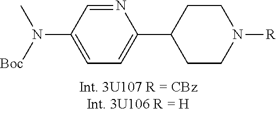

Int. 1Y4 (8.0 g), benzyl 4-(4,4,5,5-tetramethyl-1,3,2-dioxaborolan-2-yl)-3,6-dihydropyridine-1 (2H)-carboxylate (14 g), PdDPPFCl₂-DCM (1.1 g), and NaHCO₃ (7.0 g) were stirred 16 h in THF/H₂O (8:1, 90 mL) at 100° C. under an inert atmosphere. The mixture was filtered. The OL (water/EtOAc) was dried, concentrated, and purified by FC (PE/EtOAc 4:1) to give Int. 3U107 benzyl 5-((tert-butoxy-carbonyl)(methyl)amino)-3′,6′-dihydro-[2,4′-bipyridine]-1′(2′H)-carboxylate (8.5). Int. 3U107 (2.5 g) and Pd/C 10% (0.10 g) were hydrogenated 16 h at 60 psi in MeOH (30 mL). The mixture was filtered and concentrated to give Int. 3U106 tert-butyl methyl(6-(piperidin-4-yl)pyridin-3-yl)-carbamate (2.0 g).

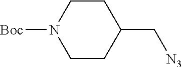 [SMILES: CC(C)(C)OC(=O)N1CCC(CN=[N+]=[N-])CC1]

NaN₃ (0.11 g) and tert-butyl 4-(bromomethyl)piperidine-1-carboxylate (0.40 g) were stirred ON in DMF (5 mL). The OL (water/EtOAc) was washed with brine, dried, and concentrated to give Int. 3T8 tert-butyl 4-(azidomethyl)piperidine-1-carboxylate (0.32 g).

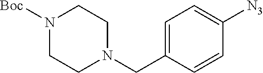 [SMILES: CC(C)(C)OC(=O)N1CCN(Cc2ccc(N=[N+]=[N-])cc2)CC1]

tert-Butyl 4-(4-bromobenzyl)piperazine-1-carboxylate (0.42 g), sodium azide (0.38 g), sodium ascorbate (40 mg), CuI (80 mg) and trans-N,N′-dimethylcyclohexane-1,2-diamine (0.12 mL) in EtOH/H₂O (7:3, 12 mL) were refluxed for 6 h under an inert atmosphere and concentrated. The OL (water/DCM) was concentrated and purified by FC (heptane/EtOAc 1:0 to 1:1) to give Int. TT23 tert-butyl 4-(4-azidobenzyl)piperazine-1-carboxylate (0.28 g).

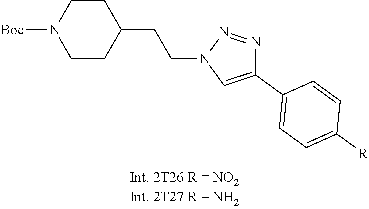

Sodium ascorbate (12 g) was added to a mixture of 1-ethynyl-4-nitrobenzene (3.0 g), tert-butyl 4-(2-azidoethyl)piperidine-1-carboxylate (5.2 g) and CuSO₄·H2O (6.5 g) in ^{t}BuOH/H₂O (1:1, 100 mL), stirred for 16 h, filtered and concentrated to give Int. 2T26 tert-butyl 4-(2-(4-(4-nitrophenyl)-1H-1,2,3-triazol-1-yl)-ethyl)piperidine-1-carboxylate (4.5 g). Fe powder (2.7 g) and NH₄Cl (5.3 g) were added to a solution of Int. 2T26 (4.0 g) in EtOH/H₂O (5:1. 60 mL) and stirred for 3 h at 90° C. and filtered. The OL (brine/EtOAc) was dried, filtered and concentrated to give Int. 2T27 tert-butyl 4-(2-(4-(4-aminophenyl)-1H-1,2,3-triazol-1-yl)-ethyl)piperidine-1-carboxylate (3.2 g).

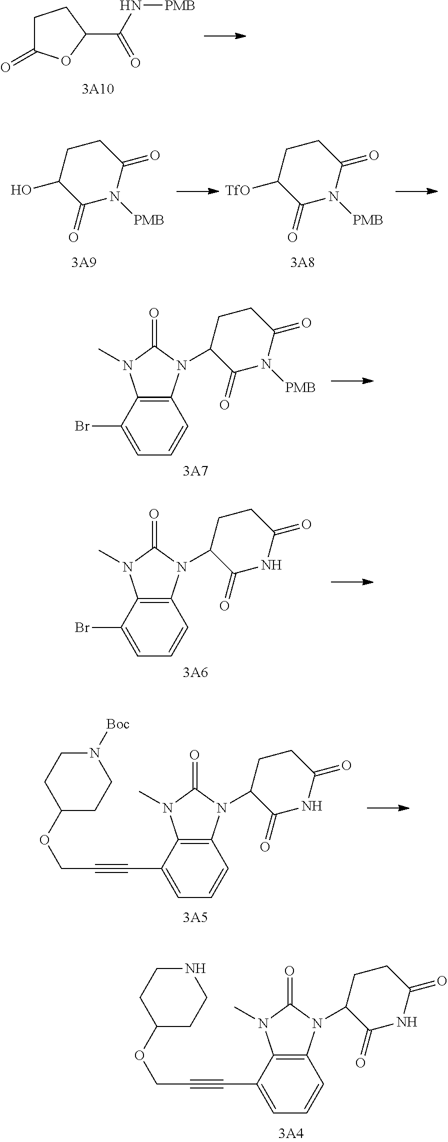

5-Oxotetrahydrofuran-2-carboxylic acid (62 g) was stirred 4 h in SOCl₂ (90 mL) at 0° C. to 80° C. and concentrated. The residue and (4-methoxy-phenyl)-methanamine (52 g) were stirred 4 h in DCM/Et₃N (1.4:1, 340 mL) at 0° C. to RT. The OL (0.5M aq. HCl/DCM) was dried, concentrated, and purified by FC (pentane/EtOAc 1:4) to give Int. 3A10 N-(4-methoxybenzyl)-5-oxotetrahydrofuran-2-carboxamide (12 g). Int. 3A10 (20 g) and KOtBu (18 g) was stirred 2 h in THF (260 mL) at −78° C. to −40° C. The OL (sat. aq. NH₄Cl/EtOAc) was washed with brine, dried, concentrated, and purified by FC (pentane/EtOAc 1:4) to give Int. 3A9 3-hydroxy-1-[(4-methoxyphenyl)-methyl]piperidine-2,6-dione (5.0 g). Tf₂O (7.1 mL) was added to a solution of Int. 3A9 (7.0 g) in DCM/pyridine (11.7:1, 76 mL) at 0° C. followed by stirring 1 h at 0° C. The OL (water/DCM) was dried, concentrated, and purified by FC (hexane/EtOAc 7:3) to give Int. 3A8 1-(4-methoxy-benzyl)-2,6-dioxopiperidin-3-yl trifluoro-methane-sulfonate (10.1 g). 7-Bromo-1-methyl-1,3-di-hydro-2H-benzo[d]imidazol-2-one (3.0 g) and KOtBu (2.1 g) were stirred 0.5 h in THF (30 mL) at 0° C. Int. 3A8 (9.1 g) was added and stirring continued 2 h at 0° C. The OL (sat. aq. NH₄Cl/EtOAc) was dried, concentrated, and purified by FC (pentane/EtOAc 7:3 to 1:1) and by trituration in MeOH/DCM/Et₂O to give Int. 3A7 3-(4-bromo-3-methyl-2-oxo-2,3-dihydro-1H-benzo[d]imidazol-1-yl)-1-(4-methoxybenzyl)piperidine-2,6-dione (2.5 g). Int. 3A7 (3.0 g) was stirred 2.5 h in TFA/TfOH (10:1, 33 mL) at RT to 80° C., concentrated, and triturated in pentane/Et₂O to give Int. 3A6 3-(4-bromo-3-methyl-2-oxo-2,3-dihydro-1H-benzo[d]-imidazol-1-yl)piperidine-2,6-dione (2.0 g). Int. 3A6 (1.5 g), tert-butyl 4-(prop-2-yn-1-yloxy)piperidine-1-carboxylate (1.6 g), Pd(PPh₃)₂Cl₂ (0.31 g), CuI (0.25 g), Cs₂CO₃ (3.6 g), and 4 Å MS were degassed in DMF (3 mL) and stirred 4 h at 80° C. The OL (water/EtOAc) was washed with brine, dried, and concentrated. The residue was combined with another batch prepared similarly on 0.1 g scale and purified by FC (pentane/EtOAc 7:3 to 1:1) to give Int. 3A5 tert-butyl 4-((3-(1-(2,6-dioxo-piperidin-3-yl)-3-methyl-2-oxo-2,3-dihydro-1H-benzo[d]imidazol-4-yl)prop-2-yn-1-yl)oxy)piperidine-1-carboxylate (0.60 g). Int. 3A5 (0.5 g) was stirred 3 h in DCM/TFA (4:1; 25 mL), concentrated, and triturated in Et₂O to give Int. 3A4 3-(3-methyl-2-oxo-4-(3-(piperidin-4-yloxy)prop-1-yn-1-yl)-2,3-dihydro-1H-benzo[d]imidazol-1-yl)piperidine-2,6-dione (0.50 g).

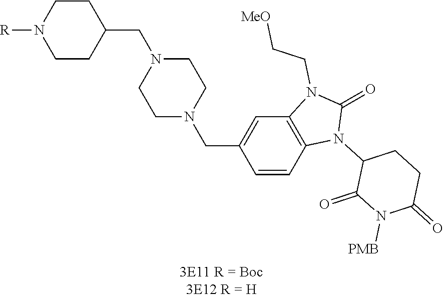

Ints. 3E13/3E5 (0.12 g/0.11 g) were stirred 0.5 h in DCE/DIPEA (87:1, 4.05 mL) at 70° C. STAB (0.28 g) was added and stirring continued 4 h. The OL (water/DCM) was washed with brine, dried, concentrated, and purified by FC (DCM/MeOH 1:0 to 4:1) to give Int. 3E11 tert-butyl 4-((4-((1-(1-(4-methoxy-benzyl)-2,6-dioxopiperidin-3-yl)-3-(2-methoxyethyl)-2-oxo-2,3-dihydro-1H-benzo[d]imidazol-5-yl)methyl)-piperazin-1-yl)methyl)-piperidine-1-carbo-xylate (0.14 g). This material was stirred 1 h in DCM/TFA (3:2; 5 mL) and concentrated to give Int. 3E12 1-(4-methoxybenzyl)-3-(3-(2-methoxyethyl)-2-oxo-5-((4-(piperidin-4-ylmethyl)piperazin-1-yl)methyl)-2,3-dihydro-1H-benzo[d]imidazol-1-yl)piperidine-2,6-dione (0.12 g, TFA salt).

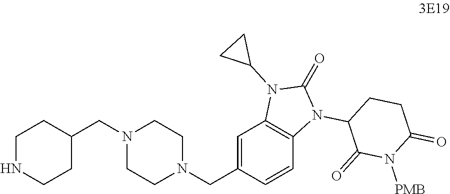

Int. 3E19 3-(3-cyclo-propyl-2-oxo-5-((4-(piperidin-4-ylmethyl)piperazin-1-yl)methyl)-2,3-dihydro-1H-benzo[d]-imidazol-1-yl)-1-(4-methoxy-benzyl)piperidine-2,6-dione (72 mg) was prepared similarly to Int. 3E1 from Ints. 3E20/3E5 (70 mg/69 mg).

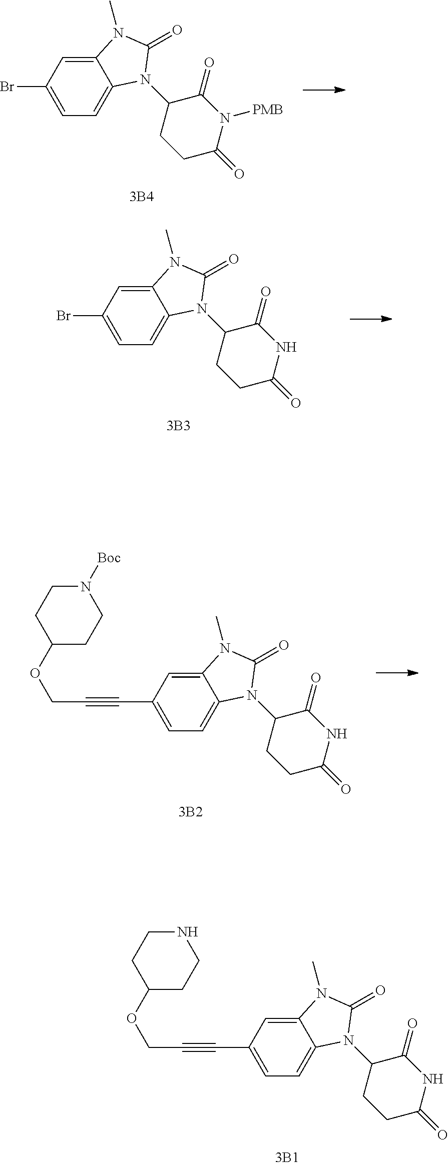

6-Bromo-1-methyl-1,3-dihydro-2H-benzo[d]imidazol-2-one (3.5 g) and KOtBu (1.9 g) were stirred 0.5 h in THF (35 mL) at 0° C. Int. 3A8 (7.1 g) was added and stirring continued 2 h. The OL (sat. aq. NH₄Cl/EtOAc) was dried, concentrated, and purified by FC (DCM/MeOH 95:5) to give Int. 3B4 3-(5-bromo-3-methyl-2-oxo-2,3-dihydro-1H-benzo[d]imidazol-1-yl)-1-(4-methoxybenzyl)piperidine-2,6-dione (6.0 g). This material was stirred 2.5 h in TFA/TfOH (1:3.3, 26 mL) at 0° C. to 80° C., concentrated, and triturated in Et₂O to give Int. 3B3 3-(5-bromo-3-methyl-2-oxo-benzimidazol-1-yl)piperidine-2,6-dione (5.0 g). Int. 3B3 (0.5 g), tert-butyl 4-(prop-2-yn-1-yloxy)-piperidine-1-carboxylate (0.5 g), PdDPPFCl₂-DCM (109 mg), CuI (56 mg), Cs₂CO₃ (1.9 g), and 4 Å MS were degas-sed in DMF (5 mL) and stirred 2 h at 80° C. The OL (water/EtOAc) was washed with brine, dried, concentrated, and purified by FC (pentane/EtOAc 3:7 to 2:3) to give Int. 3B2 tert-butyl 4-((3-(1-(2,6-dioxopiperidin-3-yl)-3-methyl-2-oxo-2,3-dihydro-1H-benzo[d]imidazol-5-yl)prop-2-yn-1-yl)oxy)piperidine-1-carboxylate (0.10 g). 85 mg of this material was stirred 1 h in DCM/TFA (4:1; 2.5 mL) at 0° C., concentrated, and HPLC-purified to give Int. 3B1 3-(3-methyl-2-oxo-5-(3-(piperidin-4-yloxy)prop-1-yn-1-yl)-2,3-dihydro-1H-benzo[d]imidazol-1-yl)piperidine-2,6-dione (20 mg).

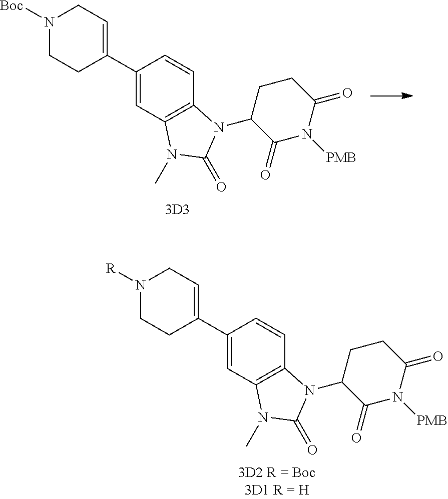

Int. 3B4 (0.15 g), tert-butyl 4-(4,4,5,5-tetramethyl-1,3,2-dioxaborolan-2-yl)-3,6-dihydro-2H-pyridine-1-carbo-xylate (0.12 g), PdDPPFCl₂-DCM (12 mg), and K₃PO₄ (0.21 g) were degassed in dioxane/water (6.6:1, 3.8 mL) and stirred 5 h at 90° C. PdDPPFCl₂-DCM (12 mg), and K₂CO₃ (0.1 g) were added and stirring continued 2 h at 90° C. The OL (brine/EtOAc) was dried, concentrated, and purified by FC (heptane/EtOAc 1:0 to 1:9) to give Int. 3D3 tert-butyl 4-[1-[1-[(4-methoxyphenyl)methyl]-2,6-dioxo-3-piperidyl]-3-methyl-2-oxo-benzimidazol-5-yl]-3,6-dihydro-2H-pyridine-1-carboxylate (0.12 g). Int. 3D3 (0.14 g) was hydrogenated ON using 10% Pd/C (14 mg) in EtOAc (1.7 mL), filtered, and concentrated to give Int. 3D2 tert-butyl 4-[1-[1-[(4-methoxyphenyl)-methyl]-2,6-dioxo-3-piperidyl]-3-methyl-2-oxo-benzimidazol-5-yl]piperidine-1-carboxylate (0.14 g). This material was stirred 1 h in 2.7M HCl in dioxane (1:2; 3 mL), concentrated, and HPLC-purified to give Int. 3D1 1-(4-methoxybenzyl)-3-(3-methyl-2-oxo-5-(piperidin-4-yl)-2,3-dihydro-1H-benzo[d]imidazol-1-yl)piperidine-2,6-dione (64 mg).

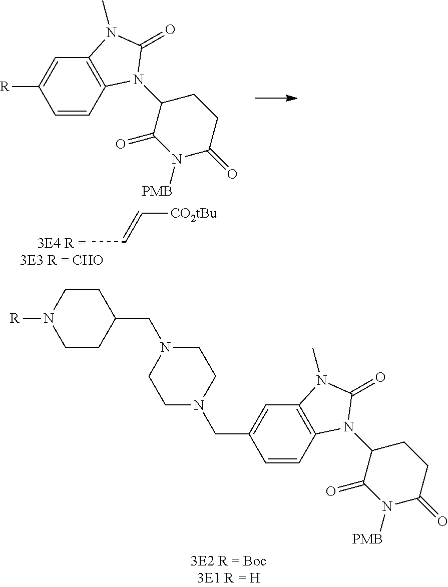

Int. 3B4 (2.5 g), tert-butyl acrylate (2.0 g), P(o-tol)₃ (0.33 g), and Pd(OAc)₂ (0.29 g) were degassed in DMF (15 mL) and Et₃N (4.1 mL) and stirred 110° C. ON. The OL (water/EtOAc) was washed with brine, dried, concentrated, and triturated in pentane to give Int. 3E4 tert-butyl (E)-3-(1-(1-(4-methoxy-benzyl)-2,6-dioxopiperidin-3-yl)-3-methyl-2-oxo-2,3-dihydro-1H-benzo[d]-imidazol-5-yl)acrylate (2.4 g). Int. 3E4 (2.5 g), NaIO₄ (2.6 g), and OsO₄ (0.25 g) were stirred ON in THF/water (25:8; 33 mL). The OL (sat. aq. NaHCO₃/EtOAc) was washed with brine, dried, concentrated, and purified by FC (pentane/EtOAc 2:3 to 3:7) to give Int. 3E3 1-(1-(4-methoxybenzyl)-2,6-dioxopiperidin-3-yl)-3-methyl-2-oxo-2,3-dihydro-1H-benzo[d]-imidazole-5-carbaldehyde (1.0 g). Ints. 3E3/3E5 (50 mg/42 mg) were stirred 0.25 h in DCE (2 mL). STAB (36 mg) was added and stirring continued 0.5 h. AcOH (1 equiv) was added and stirring continued 144 h. DIPEA (2 equiv) and STAB (83 mg) were added and stirring continued 2 h at 70° C. The OL (water/DCM) was washed with brine, dried, concentrated, and purified by FC (DCM/MeOH 1:0 to 4:1) to give Int. 3E2 tert-butyl 4-[[1-[[1-[1-[(4-methoxyphenyl)methyl]-2,6-dioxo-3-piperidyl]-3-methyl-2-oxo-benzimidazol-4-yl]methyl]-4-piperidyl]methyl]-piperazine-1-carboxylate (62 mg). This material was stirred 1 h at RT to 100° C. in TFA/TfOH (6:1; 3.5 mL) and concentrated to give Int. 3E1 3-[3-methyl-2-oxo-5-[[4-(4-piperidylmethyl)-piperazin-1-yl]methyl]-benzimidazol-1-yl]piperidine-2,6-dione (45 mg, TfOH salt).

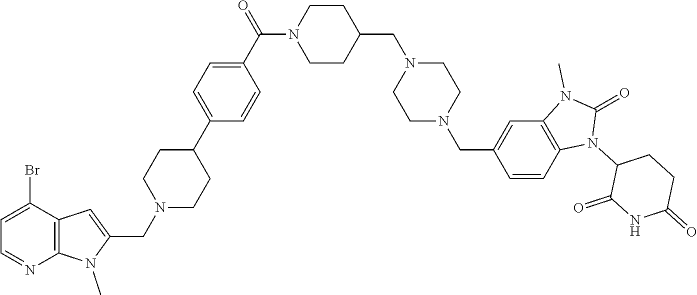 [SMILES: Cn1c(CN2CCC(c3ccc(C(=O)N4CCC(CN5CCN(Cc6ccc7c(c6)n(C)c(=O)n7C6CCC(=O)NC6=O)CC5)CC4)cc3)CC2)cc2c(Br)ccnc21]

Ints. 1AC4/3E1 (0.10 g/(0.16 g) and HATU (133 mg) were stirred ON in DMF (4 mL) and DIPEA (83 microL). The OL (2MeTHF/aq. NaHCO₃) was washed with brine, dried, concentrated, and purified by FC (DCM/MeOH 1:1 to 4:1) to give Int. 3E7 3-(5-((4-((1-(4-(1-((4-bromo-1-methyl-1H-pyrrolo[2,3-b]pyridin-2-yl)methyl)piperidin-4-yl)benzoyl)-piperidin-4-yl)methyl)piperazin-1-yl)methyl)-3-methyl-2-oxo-2,3-dihydro-1H-benzo[d]-imidazol-1-yl)piperidine-2,6-dione (154 mg).

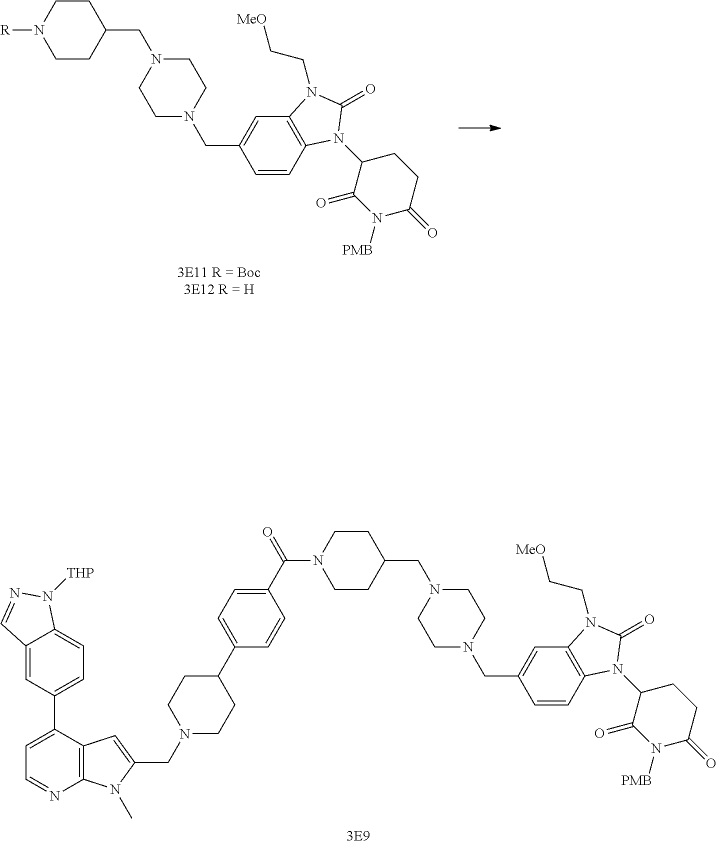

Ints. 3E13/3E5 (0.12 g/0.11 g) were stirred 0.5 h in DCE/DIPEA (87:1, 4.05 mL) at 70° C. STAB (0.28 g) was added and stirring continued 4 h. The OL (water/DCM) was washed with brine, dried, concentrated, and purified by FC (DCM/MeOH 1:0 to 4:1) to give Int. 3E11 tert-butyl 4-((4-((1-(1-(4-methoxy-benzyl)-2,6-dioxopiperidin-3-yl)-3-(2-methoxyethyl)-2-oxo-2,3-dihydro-1H-benzo[d]imidazol-5-yl)methyl)-piperazin-1-yl)methyl)-piperidine-1-carbo-xylate (0.14 g). This material was stirred 1 h in DCM/TFA (3:2; 5 mL) and concentrated to give Int. 3E12 1-(4-methoxybenzyl)-3-(3-(2-methoxyethyl)-2-oxo-5-((4-(piperidin-4-ylmethyl)-piperazin-1-yl)methyl)-2,3-dihydro-1H-benzo[d]imidazol-1-yl)piperidine-2,6-dione (0.12 g, TFA salt). 30 mg of this material, Int. 3A2 (34 mg), and HATU (24 mg) were stirred 2 h in DMF/DIPEA (23.8:1, 2.1 mL), filtered, and HPLC-purified to give Int. 3E9 1-(4-methoxy-benzyl)-3-(3-(2-methoxyethyl)-5-((4-((1-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)piperidin-4-yl)-methyl)piperazin-1-yl)methyl)-2-oxo-2,3-dihydro-1H-benzo[d]imidazol-1-yl)piperidine-2,6-dione (51 mg).

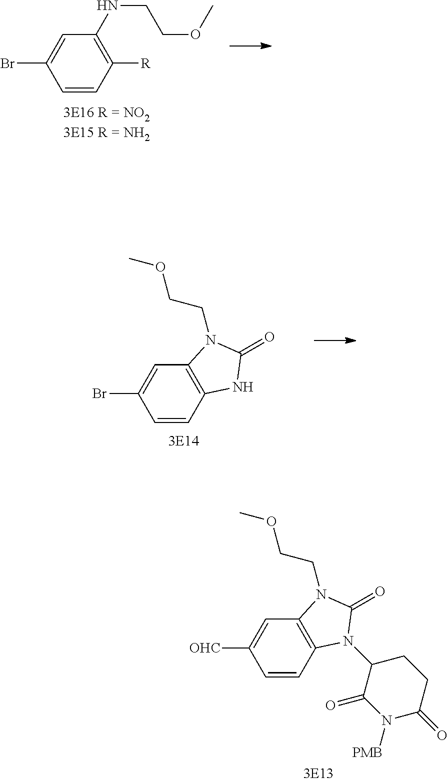

4-Bromo-2-fluoro-1-nitrobenzene (5.0 g) and 2-methoxyethan-1-amine (2.6 g) were stirred in THF (50 mL) at 0° C. to RT ON. The OL (sat. aq. NaHCO₃/EtOAc) was washed with brine, dried, concentrated, and triturated in pentane to give Int. 3E16 5-bromo-N-(2-methoxyethyl)-2-nitro-aniline (5.0 g). Int. 3E16 (9.0 g), iron powder (10.7 g), and NH₄Cl (17.5 g) were stirred 3 h in EtOH/AcOH (2:1; 90 mL) at 70-80° C. to RT. The OL (EtOAc/sat. aq. NaHCO₃) from the filtrate was dried, concentrated, and triturated in pentane to give Int. 3E15 5-bromo-N1-(2-methoxyethyl)benzene-1,2-diamine (6.8 g). 3.3 g of this material and CDI (5.5 g) were stirred ON in ACN (35 mL) at 80° C., diluted with ACN (30 mL), filtered, and concentrated. The residue was purified by FC (hexane/EtOAc 1:1 to 2:3) to give Int. 3E14 6-bromo-1-(2-methoxyethyl)-1,3-dihydro-2H-benzo[d]imidazol-2-one (1.2 g). This material was converted to Int. 3E13 1-(1-(4-methoxybenzyl)-2,6-dioxopiperidin-3-yl)-3-(2-methoxyethyl)-2-oxo-2,3-dihydro-1H-benzo[d]imidazole-5-carbaldehyde similarly to Int. 3E3.

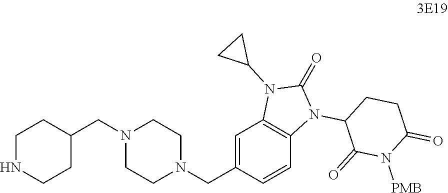

Int. 3E19. 3-(3-cyclo-propyl-2-oxo-5-((4-(piperidin-4-ylmethyl)piperazin-1-yl)methyl)-2,3-dihydro-1H-benzo[d]-imidazol-1-yl)-1-(4-methoxy-benzyl)piperidine-2,6-dione (72 mg) was prepared similarly to Int. 3E1 from Ints. 3E20/3E5 (70 mg/69 mg).

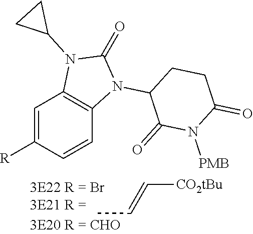

5-Bromo-3-cyclo-propyl-1H-benzimidazol-2-one (1.5 g) and KOtBu (1.9 g) were stirred 0.5 h in THF (25 mL) before Int. 3A8 (4.6 g) and THF (10 mL) were added and stirring continued 3 h at 0° C. The mixture was combined with another batch prepared similarly and purified by FC (hexane/EtOAc 1:1 to 2:3) and by trituration in pentane to give Int. 3E22 3-(5-bromo-3-cyclo-propyl-2-oxo-2,3-dihydro-1H-benzo[d]imidazol-1-yl)-1-(4-methoxy-benzyl)piperidine-2,6-dione (1.15 g). 0.4 g of this material was converted to Int. 3E21 (E)-3-(3-cyclo-propyl-5-(4,4-dimethyl-3-oxopent-1-en-1-yl)-2-oxo-2,3-dihydro-1H-benzo[d]imidazol-1-yl)-1-(4-methoxybenzyl)-piperidine-2,6-dione (0.41 g) similarly as t. 3E4. Int. 3E20 3-cyclo-Propyl-1-(1-(4-methoxybenzyl)-2,6-dioxo-piperidin-3-yl)-2-oxo-2,3-dihydro-1H-benzo[d]imidazole-5-carbaldehyde was prepared similarly to Int. 3E3 from Int. 3E21.

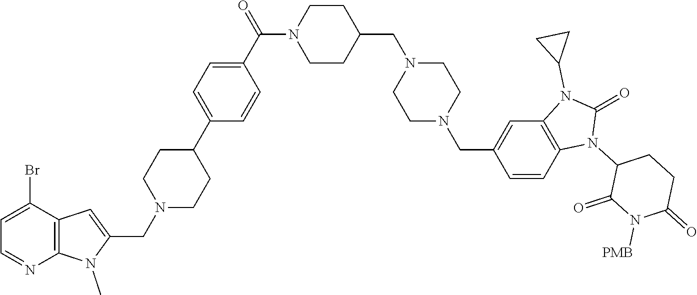

Ints. 1AC4/3E19 (33 mg/36 mg) and HATU (30 mg) were stirred 2 h in DMF/DIPEA (201:, 2.1 mL). The filtrate after filtration was HPLC-purified to give Int. 3E25 3-(5-((4-((1-(4-(1-((4-bromo-1-methyl-1H-pyrrolo[2,3-b]pyridin-2-yl)methyl)piperidin-4-yl)benzoyl)piperidin-4-yl)methyl)piperazin-1-yl)methyl)-3-cyclopropyl-2-oxo-2,3-dihydro-1H-benzo[d]imidazol-1-yl)-1-(4-methoxybenzyl)piperidine-2,6-dione (33 mg).

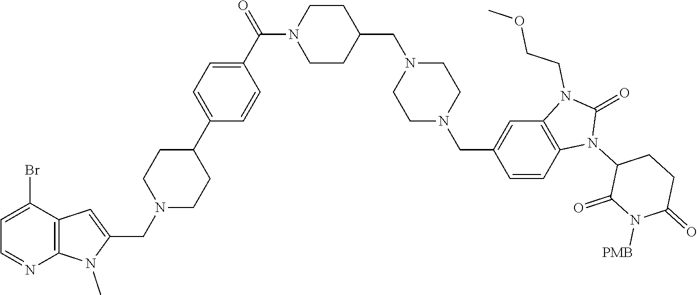

Ints. 3E12/1AC4 (80 mg/72 mg) and HATU (64 mg) were stirred 2 h in DMF (4 mL) and DIPEA (0.23 mL). The OL (water/EtOAc) was washed with brine, dried, concentrated, and purified by FC (DCM/MeOH 1:0 to 4:1) and by HPLC to give Int. 3E26 3-(5-((4-((1-(4-(1-((4-bromo-1-methyl-1H-pyrrolo[2,3-b]pyridin-2-yl)methyl)-piperidin-4-yl)benzoyl)-piperidin-4-yl)methyl)piperazin-1-yl)methyl)-3-(2-methoxyethyl)-2-oxo-2,3-dihydro-1H-benzo[d]imidazol-1-yl)-1-(4-methoxybenzyl)piperidine-2,6-dione (40 mg).

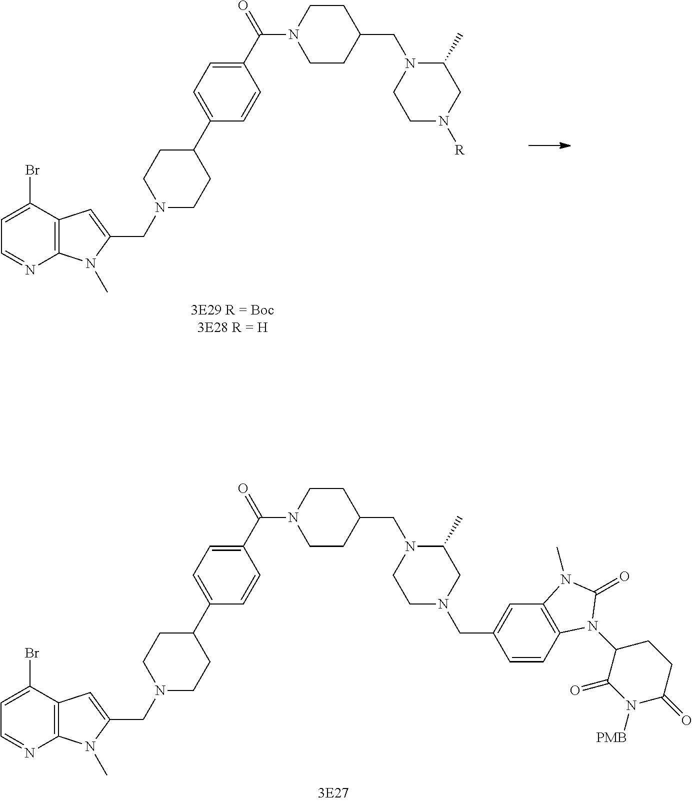

Ints. 3E30/1AC4 (58 mg/70 mg) and HATU (87 mg) were stirred 3 h in DMF/DIPEA (28.1:1, 1.66 mL). The OL (EtOAc/water) was washed with brine, dried, concentrated, and HPLC-purified to give Int. 3E29 tert-butyl (R)-4-((1-(4-(1-((4-bromo-1-methyl-1H-pyrrolo[2,3-b]pyridin-2-yl)-methyl)piperidin-4-yl)benzoyl)piperidin-4-yl)methyl)-3-methyl-piperazine-1-carboxylate (60 mg). This material was stirred 0.5 h in DCM/TFA (2:1; 3 mL). The residue after concentration was SCX-purified to give Int. 3E28 (R)-(4-(1-((4-bromo-1-methyl-1H-pyrrolo[2,3-b]pyridin-2-yl)methyl)piperidin-4-yl)phenyl)(4-((2-methyl-piperazin-1-yl)methyl)-piperidin-1-yl)methanone (40 mg). Ints. 3E28/3E3 (39 mg/22 mg), and STAB (57 mg) were stirred 1 h in DCE/DIPEA (111:1, 2.02 mL) at 70° C. STAB (57 mg) was added and stirring continued ON. STAB (57 mg) was added and stirring continued ON. The OL (water/EtOAc) was washed with brine, dried, concentrated, and purified by FC (DCM/MeOH/Et₃N 94.5:5:0.5) to give Int. 3E27 3-(5-(((R)-4-((1-(4-(1-((4-bromo-1-methyl-1H-pyrrolo[2,3-b]pyridin-2-yl)methyl)piperidin-4-yl)benzoyl)piperidin-4-yl)methyl)-3-methylpiperazin-1-yl)methyl)-3-methyl-2-oxo-2,3-dihydro-1H-benzo[d]imidazol-1-yl)-1-(4-methoxybenzyl)piperidine-2,6-dione (42 mg).

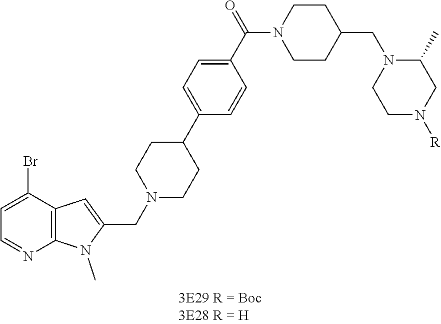

Ints. 3E30/1AC4 (58 mg/70 mg) and HATU (87 mg) were stirred 3 h in DMF/DIPEA (28.1:1, 1.66 mL). The OL (EtOAc/water) was washed with brine, dried, concentrated, and HPLC-purified to give Int. 3E29 tert-butyl (R)-4-((1-(4-(1-((4-bromo-1-methyl-1H-pyrrolo[2,3-b]pyridin-2-yl)-methyl)piperidin-4-yl)benzoyl)piperidin-4-yl)methyl)-3-methyl-piperazine-1-carboxylate (60 mg). This material was stirred 0.5 h in DCM/TFA (2:1; 3 mL). The residue after concentration was SCX-purified to give Int. 3E28 (R)-(4-(1-((4-bromo-1-methyl-1H-pyrrolo[2,3-b]pyridin-2-yl)methyl)piperidin-4-yl)phenyl)(4-((2-methyl-piperazin-1-yl)methyl)-piperidin-1-yl)methanone (40 mg).

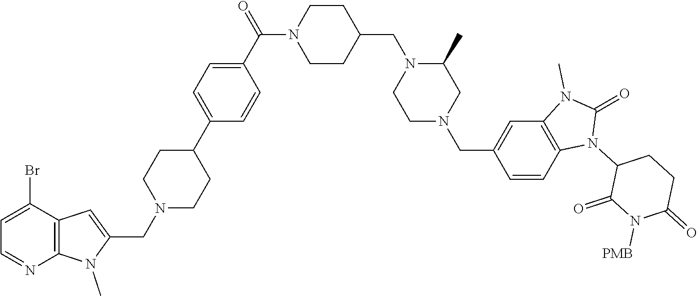

Int. 3E32. 3-(5-(((S)-4-((1-(4-(1-((4-bromo-1-methyl-1H-pyrrolo[2,3-b]pyridin-2-yl)methyl)piperidin-4-yl)benzoyl)piperidin-4-yl)methyl)-3-methylpiperazin-1-yl)methyl)-3-methyl-2-oxo-2,3-dihydro-1H-benzo[d]imidazol-1-yl)-1-(4-methoxybenzyl)piperidine-2,6-dione was prepared in a similarly to Int. 3E27 from tert-butyl (S)-3-methyl-piperazine-1-carboxylate.

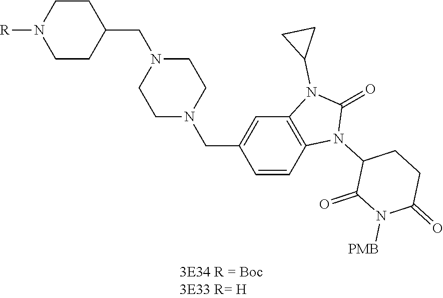

Ints. 3E20/3E5 (0.70 g/0.60 g) were stirred 6 h in DCE/DIPEA (10.9:1, 27.3 mL) at 0° C. to RT. STAB (1.4 g) was added and stirring continued ON. The OL (water/DCM) was washed with brine, dried, concentrated, and purified by FC (DCM/MeOH 97:3 to 96:4) to give Int. 3E34 tert-butyl 4-((4-((3-cyclo-propyl-1-(1-(4-methoxy-benzyl)-2,6-dioxo-piperidin-3-yl)-2-oxo-2,3-dihydro-1H-benzo[d]imidazol-5-yl)methyl)piperazin-1-yl)-methyl)-piperidine-1-carboxylate (0.6 g). This material was stirred 48 h in DCE/TFA/TfOH (33:1:10. 26.6 mL) at 60° C., concentrated, and triturated in Et₂O/pentane to give Int. 3E33 3-(3-cyclo-propyl-2-oxo-5-((4-(piperidin-4-ylmethyl)-piperazin-1-yl)methyl)-2,3-dihydro-1H-benzo[d]imidazol-1-yl)piperidine-2,6-dione (0.4 g).

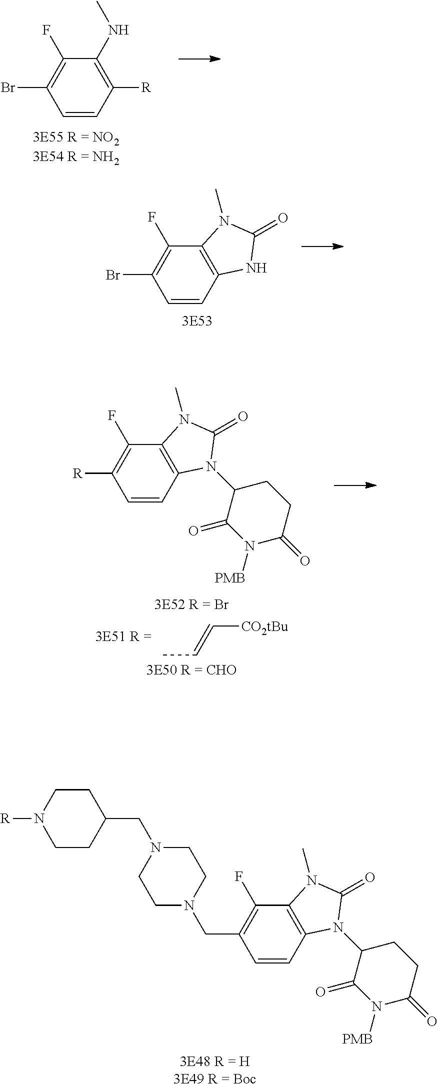

1-Bromo-2,3-difluoro-4-nitrobenzene (3.0 g) was stirred 2 h in 0.6M in THF (43 mL) at 0° C. The OL (sat. aq. NaHCO₃/EtOAc) was washed with brine, dried, and concentrated to give Int. 3E55 3-bromo-2-fluoro-N-methyl-6-nitro-aniline (2.7 g). Int. 3E55 (3.0 g), NH₄Cl (6.4 g), and iron powder (3.4 g) were stirred 8 h in EtOH/water (1.7:1, 8 mL), filtered, and concentrated. The OL (sat. aq. NaHCO₃/EtOAc) was dried, concentrated, and purified by FC (hexane/EtOAc 3:1) to give Int. 3E54 4-bromo-3-fluoro-N2-methyl-benzene-1,2-diamine (1.9 g). 1.0 g of this material and CDI (1.1 g) were stirred ON in ACN (5 mL) at 90° C., diluted with water to precipitate Int. 3E53 5-bromo-4-fluoro-3-methyl-1H-benzimidazol-2-one (0.41 g). Int. 3E53 (0.60 g) and KOtBu (0.36 g) were stirred 0.5 h in THF (10 mL) at 0° C. Int. 3A8 (2.46 g) was added and stirring continued 4 h at 0° C. The OL (sat. aq. NH₄Cl/EtOAc) was dried, concentrated, and purified by FC (pentane/EtOAc 1:0 to 1:1) to give Int. 3E52 3-(5-bromo-4-fluoro-3-methyl-2-oxo-2,3-dihydro-1H-benzo[d]-imidazol-1-yl)-1-(4-methoxybenzyl)piperidine-2,6-dione (0.27 g). This material, tert-butyl acrylate (0.2 mL), Pd(OAc)₂ (12 mg), and P(o-tol)₃ (33 mg) were degassed in DMF/Et₃N (17.4:1, 4.2 mL) and stirred ON at 120° C. tert-Butyl acrylate (0.1 mL), Pd(OAc)₂ (6 mg), and P(o-tol)₃ (15 mg) were added and stirring continued 4 h at 120° C. The OL (water/EtOAc) was washed with brine, dried, concentrated, and purified by FC (heptane/EtOAc 1:0 to 0:1) and further by HPLC to give Int. 3E51 tert-butyl (E)-3-(4-fluoro-1-(1-(4-methoxybenzyl)-2,6-dioxo-piperidin-3-yl)-3-methyl-2-oxo-2,3-dihydro-1H-benzo[d]imidazol-5-yl)acrylate (99 mg). Int. 3E51 (95 mg) and NaIO₄ (97 mg) were stirred ON in THF/water (2:1; 6 mL) and 2.5% OsO₄ in tBuOH (23 microL). The OL (2MeTHF/water) was washed with 5% aq. Na₂S₂O₃ and brine, dried, concentrated, and purified by FC (heptane/EtOAc 1:0 to 0:1) and further by HPLC to give Int. 3E50 4-fluoro-1-(1-(4-methoxybenzyl)-2,6-dioxo-piperidin-3-yl)-3-methyl-2-oxo-2,3-dihydro-1H-benzo[d]imidazole-5-carbaldehyde (68 mg). This material, Int. 3E5 (54 mg), and 4 Å MS were stirred 0.25 h in DCE (5 mL) at RT and 0.25 h at 40° C. STAB (169 mg) was added and stirring continued 4 h at 40° C. The OL (water/DCM) was washed with brine, dried, concentrated, and purified by FC (DCM/MeOH 1:0 to 9:1) to give Int. 3E49 tert-butyl 4-((4-((4-fluoro-1-(1-(4-methoxybenzyl)-2,6-dioxo-piperidin-3-yl)-3-methyl-2-oxo-2,3-dihydro-1H-benzo[d]imidazol-5-yl)methyl)-piperazin-1-yl)methyl)-piperidine-1-carboxylate (86 mg). 80 mg of this material was stirred 1 h in DCM/TFA (2:1; 3 mL) and concentrated to give Int. 3E48 3-(4-fluoro-3-methyl-2-oxo-5-((4-(piperidin-4-ylmethyl)piperazin-1-yl)methyl)-2,3-dihydro-1H-benzo[d]imidazol-1-yl)-1-(4-methoxybenzyl)piperidine-2,6-dione (0.10 g, TFA salt).

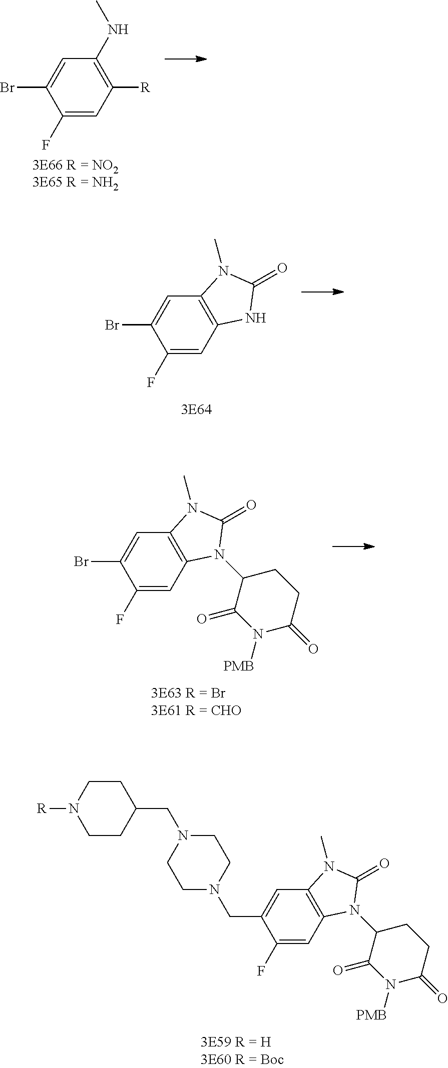

1-Bromo-2,5-difluoro-4-nitrobenzene (3.0 g) was stirred 2 h 0.9M H₂NCH₃ in THF (27.6 mL) at 0° C. and concentrated. The OL (water/EtOAc) was dried and concentrated to give Int. 3E66 5-bromo-4-fluoro-N-methyl-2-nitro-aniline (2.8 g). This material, NH₄Cl (5.8 g), and Iron powder (3.0 g) were stirred 3 h in EtOH/water at 80° C. and filtered. The OL (water/EtOAc) was washed with sat. aq. NaHCO₃, dried, and concentrated to give Int. 3E65 4-bromo-5-fluoro-N2-methyl-benzene-1,2-diamine (2.1 g). 1.5 g of this material and CDI (2.8 g) were stirred ON in ACN (20 mL) at 80° C., concentrated, and triturated in water (10 mL) to give Int. 3E64 5-bromo-6-fluoro-3-methyl-1H-benzimidazol-2-one (1.0 g). Int. 3E64 (1.0 g) and KOtBu (0.64 g) were stirred 0.5 h in THF (10 mL) at 0° C. Int. 3A8 (2.6 g) was added and stirring continued 3 h at 0° C. The OL (sat. aq. NH₄Cl/EtOAc) was dried, concentrated, and purified by FC (pentane/EtOAc 1:0 to 2:3) to give Int. 3E63 3-(5-bromo-6-fluoro-3-methyl-2-oxo-benzimidazol-1-yl)-1-[(4-methoxyphenyl)methyl]-piperidine-2,6-dione (0.29 g). 280 mg of this material, tert-butyl acrylate (0.22 mL), Pd(OAc)₂ (13 mg), and P(o-tol)₃ (36 mg) were degassed in DMF/Et₃N (16.7:1, 4.24 mL) and stirred ON at 120° C. The OL (water/EtOAc) was washed with brine, dried, concentrated, and purified by FC (heptane/EtOAc 1:0 to 1:4) to give Int. 3E62 tert-butyl (E)-3-(6-fluoro-1-(1-(4-methoxybenzyl)-2,6-dioxo-piperidin-3-yl)-3-methyl-2-oxo-2,3-dihydro-1H-benzo[d]imidazol-5-yl)acrylate (48 mg). This material and NaIO₄ (59 mg) were stirred ON in THF/water (2:1; 3 mL) and 2.5% OsO₄ in tBuOH (23 microL). The OL (2MeTHF/water) was washed with 5% aq. Na₂S₂O₃ and brine, dried, concentrated, and purified by FC (heptane/EtOAc 1:0 to 0:1) to give Int. 3E61 6-fluoro-1-(1-(4-methoxybenzyl)-2,6-dioxopiperidin-3-yl)-3-methyl-2-oxo-2,3-dihydro-1H-benzo[d]-imidazole-5-carbaldehyde (28 mg). This material and Int. 3E5 (28 mg) were stirred 0.3 h in DCE (2 mL) at RT and 0.3 h at 40° C. STAB (70 mg) was added and stirring continued 3 h at 40° C. The OL (water/DCM) was washed with brine, dried, concentrated, and HPLC-purified to Int. 3E60 tert-butyl 4-((4-((6-fluoro-1-(1-(4-methoxybenzyl)-2,6-dioxopiperidin-3-yl)-3-methyl-2-oxo-2,3-dihydro-1H-benzo[d]imidazol-5-yl)methyl)-piperazin-1-yl)methyl)piperidine-1-carboxylate (41 mg).

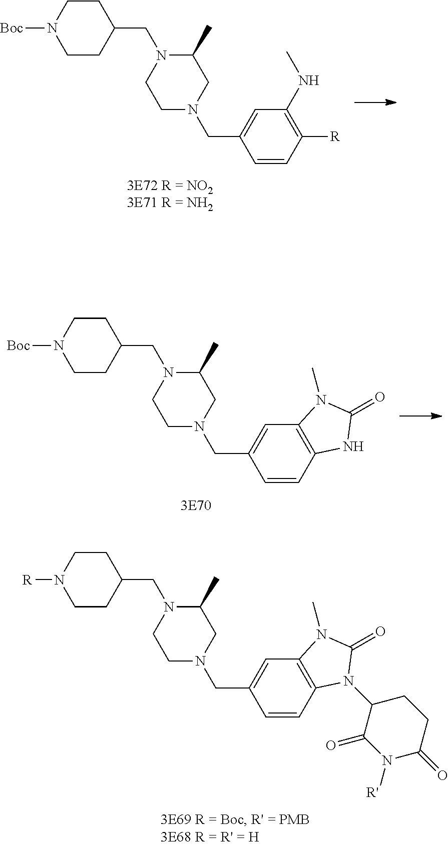

Int. 3E73 (1.8 g) was stirred 6 h in 1M H₂NCH₃ in THF (20 mL) at 0° C. to RT and concentrated to give Int. 3E72 tert-butyl 4-[[(2S)-2-methyl-4-[[3-(methylamino)-4-nitrophenyl]-methyl]piperazin-1-yl]methyl]piperidine-1-carboxylate (1.5 g). Int. 3E72 (2.3 g) was hydrogenated ON using 10% Pd/C (0.5 g) THF/EtOAc (1:1 mL. 20 mL). Filtration, concentration, and purification by FC (pentane/EtOAc 1:0 to 1:9) to give Int. 3E71 tert-butyl 4-[[(2S)-4-[[4-amino-3-(methylamino)phenyl]methyl]-2-methyl-piperazin-1-yl]-methyl]-piperidine-1-carboxylate (1.8 g). This material and CDI (1.0 g) were stirred ON in ACN (20 mL) at 80° C. The mixture was concentrated. The OL (water/EtOAc) was dried, concentrated, and purified by FC (pentane/EtOAc 1:0 to 1:9) to give Int. 3E70 tert-butyl 4-[[(2S)-2-methyl-4-[(3-methyl-2-oxo-1H-benzimidazol-5-yl)methyl]-piperazin-1-yl]methyl]-piperidine-1-carboxylate (1.2 g). 0.55 g of this material and KOtBu (189 mg) were stirred 0.5 h in THF (3 mL) at 0° C. Int. 3A8 (0.8 g) was added and stirring continued 4 h at 0° C. The OL (sat. aq. NH₄Cl/EtOAc) was dried, concentrated, and purified by FC (DCM/MeOH 1:0 to 95:5) to give Int. 3E69 tert-butyl 4-[[(2S)-4-[[1-[1-[(4-methoxyphenyl)-methyl]-2,6-dioxo-3-piperidyl]-3-methyl-2-oxo-benz-imidazol-5-yl]methyl]-2-methyl-piperazin-1-yl]methyl]-piperidine-1-carboxylate (0.50 g). This material was stirred 2 h in TFA/TfOH (5:1; 1.2 mL) at 80° C., concentrated, and triturated in water/Et₂O to give Int. 3E68 3-[3-methyl-5-[[(3S)-3-methyl-4-(4-piperidyl-methyl)piperazin-1-yl]methyl]-2-oxo-benzimidazol-1-yl]-piperidine-2,6-dione (0.35 g).

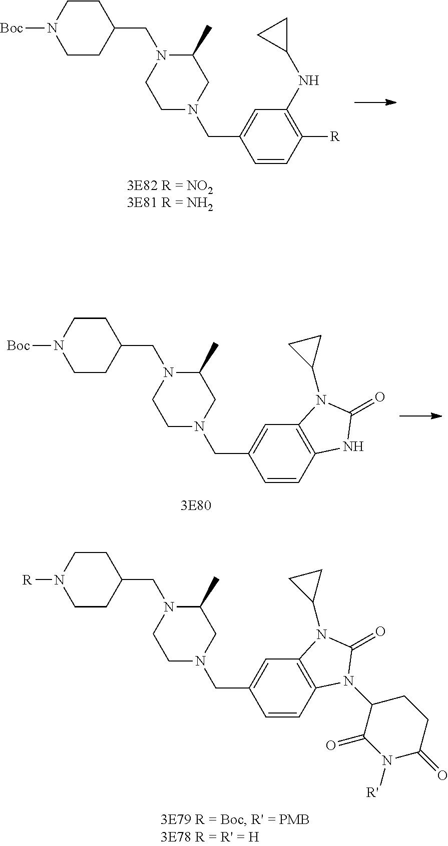

Int. 3E73 (5.0 g) and cyclo-propyl amine (3.2 g) were stirred ON in THF (50 mL) at 0° C. to RT. The residue after concentration was purified by FC (pentane/EtOAc 7:3) to give Int. 3E82 tert-butyl 4-[[(2S)-4-[[3-(cyclo-propyl-amino)-4-nitro-phenyl]methyl]-2-methyl-piperazin-1-yl]methyl]piperidine-1-carboxylate (4.5 g). This material was hydrogenated 4 h using 10% Pd/C (2.0 g) in MeOH (50 mL), filtered, and concentrated to give Int. 3E81 tert-butyl 4-[[(2S)-4-[[4-amino-3-(cyclopropylamino)phenyl]methyl]-2-methyl-piperazin-1-yl]-methyl]-piperidine-1-carboxylate (3.0 g). This material and CDI (1.5 g) were stirred ON in ACN (30 mL) at 80° C. and concentrated. The OL (water/EtOAc) was dried, concentrated, and purified by FC (pentane/EtOAc 1:4 to 1:9) to give Int. 3E80 tert-butyl 4-[[(2S)-4-[(3-cyclo-propyl-2-oxo-1H-benzimidazol-5-yl)methyl]-2-methyl-piperazin-1-yl]methyl]-piperidine-1-carboxylate (1.65 g). 1.0 g of this material and KOtBu (0.35 g) were stirred 0.5 h in THF (15 mL) at 0° C. Int. 3A8 (1.6 g) was added and stirring continued 4 h at 0° C. The OL (sat. aq. NH₄Cl/EtOAc) was washed with brine, dried, concentrated, and purified by FC (pentane/EtOAc 1:1 to 2:3) to give Int. 3E79 tert-butyl 4-[[(2S)-4-[[3-cyclo-propyl-1-[1-[(4-methoxyphenyl)methyl]-2,6-dioxo-3-piperidyl]-2-oxo-benzimidazol-5-yl]methyl]-2-methyl-piperazin-1-yl]-methyl]-piperidine-1-carboxylate (0.7 g). A solution of 0.12 g of this material in DCE/TFA/TfOH (40:10:1; 5.3 mL) was stirred ON at 60° C. and concentrated to give Int. 3E78 3-(3-cyclo-propyl-5-(((S)-3-methyl-4-(piperidin-4-ylmethyl)piperazin-1-yl)-methyl)-2-oxo-2,3-dihydro-1H-benzo[d]imidazol-1-yl)piperidine-2,6-dione (0.11 g).

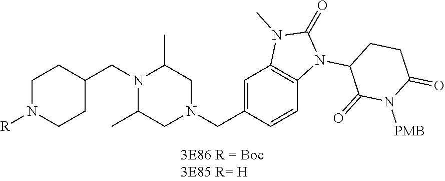

Ints. 3E87/3E3 (0.15 g/0.20 g) were stirred 3 h in DCE/DIPEA (10:2, 2.75 mL). STAB (0.78 g) was added and stirring continued ON. The OL (water/DCM) was dried, concentrated, and purified by FC (DCM/MeOH 95:5 to 9:1) to give Int. 3E86 tert-butyl 4-[[4-[[1-[1-[(4-methoxyphenyl)methyl]-2,6-dioxo-3-piperidyl]-3-methyl-2-oxo-benzimidazol-5-yl]methyl]-2,6-cis-dimethyl-piperazin-1-yl]-methyl]piperidine-1-carboxylate (0.15 g). This material (0.15 g) was stirred 2 h in DCM/4M HCl in dioxane (2:1; 3 mL), concentrated, and triturated in DCM to give Int. 3E85 3-[5-[[3,5-cis-dimethyl-4-(4-piperidyl-methyl)-piperazin-1-yl]methyl]-3-methyl-2-oxo-benzimidazol-1-yl]-1-[(4-methoxyphenyl)methyl]piperidine-2,6-dione (0.1 g, HCl salt).

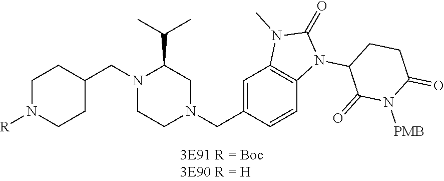

Ints. 3E92/3E3 (0.23 g/0.32 g) were stirred 6 h in DCE/DIPEA (28:1, 10.4 mL). STAB (1.13 g) was added and stirring continued ON. The OL (water/DCM) was dried, concentrated, and purified by FC (DCM/MeOH 95:5 to 9:1) to give Int. 3E91 tert-butyl 4-[[(2S)-2-iso-propyl-4-[[1-[1-[(4-methoxyphenyl)methyl]-2,6-dioxo-3-piperidyl]-3-methyl-2-oxo-benzimidazol-5-yl]methyl]piperazin-1-yl]methyl]piperidine-1-carboxylate (0.2 g). This material was stirred 2 h in DCM/4M HCl in dioxane (5:4; 9 mL) and concentrated to give Int. 3E90 3-[5-[[(3S)-3-iso-propyl-4-(4-piperidylmethyl)piperazin-1-yl]-methyl]-3-methyl-2-oxo-benzimidazol-1-yl]-1-[(4-methoxyphenyl)methyl]-piperidine-2,6-dione (0.16 g, HCl salt).

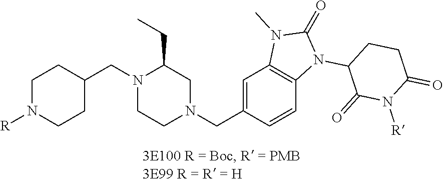

Ints. 3E3/3E101 (0.27 g/0.25 g) were stirred 6 h in DCE/DIPEA (8.6:1, 5.4 mL). STAB (0.35 g) was added and stirring continued ON. The OL (water/DCM) was dried, concentrated, and purified by FC (hexane/EtOAc 1:4) to give Int. 3E100 tert-butyl 4-[[(2S)-2-ethyl-4-[[1-[1-[(4-methoxyphenyl)methyl]-2,6-dioxo-3-piperidyl]-3-methyl-2-oxo-benz-imidazol-5-yl]-methyl]piperazin-1-yl]methyl]piperidine-1-carboxylate (0.20 g). This material was stirred ON in TFA/TfOH (10:1; 2 mL) at 0-80° C. and concentrated to give Int. 3E99 3-(5-(((S)-3-ethyl-4-(piperidin-4-ylmethyl)piperazin-1-yl)methyl)-3-methyl-2-oxo-2,3-dihydro-1H-benzo[d]imidazol-1-yl)piperidine-2,6-dione (0.10 g, TfOH salt).

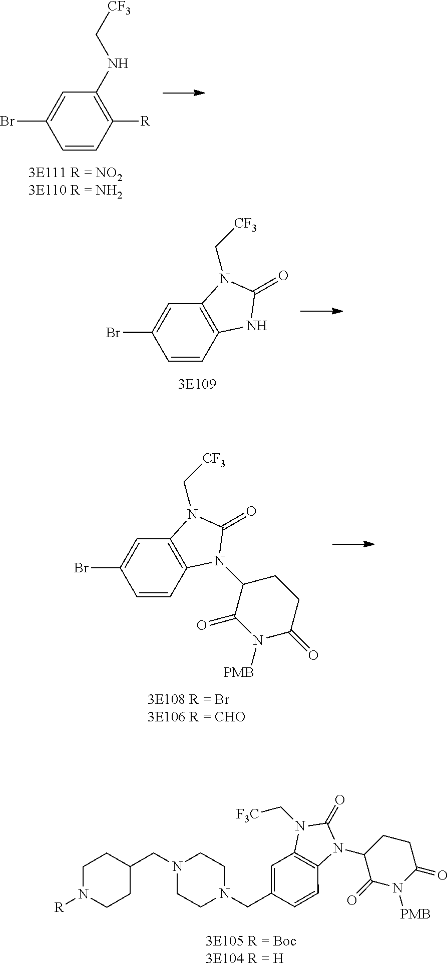

4-Bromo-2-fluoro-1-nitrobenzene (5.0 g) and 2,2,2-trifluoroethanamine (6.2 g, HCl salt) were stirred ON in THF/DIPEA (1.3:1, 36 mL) at 80° C. The OL (water/EtOAc) was washed with brine, dried, concentrated, and purified by FC (pentane/EtOAc 4:1 to 1:1) to give Int. 3E111 5-bromo-2-nitro-N-(2,2,2-trifluoroethyl)aniline (6.4 g). 5.0 g of this material, NH₄Cl (8.9 g), and iron powder (4.6 g) were stirred ON in EtOH/water (2:1; 30 mL) at 70° C. and filtered. The OL (water/EtOAc) was dried and concentrated to give Int. 3E110 4-bromo-N2-(2,2,2-trifluoroethyl)benzene-1,2-diamine (2.3 g). This material and CDI (1.62 g) were stirred ON in ACN (5 mL) at 0° C. to 80° C., concentrated and triturated in water to give Int. 3E109 5-bromo-3-(2,2,2-trifluoro-ethyl)-1H-benzimida-zol-2-one (1.2 g). 1.0 g of this material and KOtBu (0.57 g) were stirred 0.5 h in THF (10 mL) at 0° C. Int. 3E3 (2.2 g) was added and stirring continued 4 h at 0° C. The OL (water/EtOAc) was dried, concentrated, and purified by FC (pentane/EtOAc 7:3 to 1:1) to give Int. 3E108 3-[5-bromo-2-oxo-3-(2,2,2-trifluoro-ethyl)benzimidazol-1-yl]-1-[(4-methoxyphenyl)methyl]piperidine-2,6-dione (1.0 g). 0.3 g of this material, tert-butyl acrylate, Pd(OAc)₂ (29 mg), and P(o-tol)₃ (33 mg) were degassed in DMF/Et₃N (27.3:1, 6.2 mL) and stirred ON at 110° C. The OL (water/EtOAc) was washed with brine, dried, concentrated, and purified by FC (hexane/EtOAc 9:1 to 85:15) to give Int. 3E107 tert-butyl (E)-3-[1-[1-[(4-methoxyphenyl)-methyl]-2,6-dioxo-3-piperidyl]-2-oxo-3-(2,2,2-trifluoroethyl)-benzimidazol-5-yl]prop-2-enoate (0.26 g). This material, NaIO₄ (0.14 g), and OsO₄ (14 mg) were stirred ON in THF/water (3:1; 20 mL). The OL (sat. aq. Na₂S₂O₃ and sat. aq. NaHCO₃/EtOAc) was washed with brine, dried, and concentrated to give Int. 3E106 1-[1-[(4-methoxyphenyl)-methyl]-2,6-dioxo-3-piperidyl]-2-oxo-3-(2,2,2-trifluoro-ethyl)benzimidazole-5-carb-aldehyde (0.24 g). Ints. 3E5/3E106 (76 mg/0.24 g) were stirred 6 h in DCE/DIPEA (86:1, 12.1 mL). STAB (0.23 g) was added and stirring continued ON at 0° C. to RT. The OL (water/EtOAc) was washed with brine, dried, concentrated, and purified by FC (DCM/MeOH 97:3 to 95:5) to give Int. 3E105 tert-butyl 4-[[4-[[1-[1-[(4-methoxyphenyl)methyl]-2,6-dioxo-3-piperidyl]-2-oxo-3-(2,2,2-trifluoroethyl)benzimidazol-5-yl]methyl]-piperazin-1-yl]methyl]piperidine-1-carboxylate (0.19 g), which was stirred 4 h in DCM/4M HCl in dioxane (5:2; 7 mL) and concentrated to give Int. 3E104 1-[(4-methoxyphenyl)methyl]-3-[2-oxo-5-[[4-(4-piperidyl-methyl)piperazin-1-yl]methyl]-3-(2,2,2-trifluoroethyl)benzimidazol-1-yl]piperidine-2,6-dione (0.16 g, HCl salt).

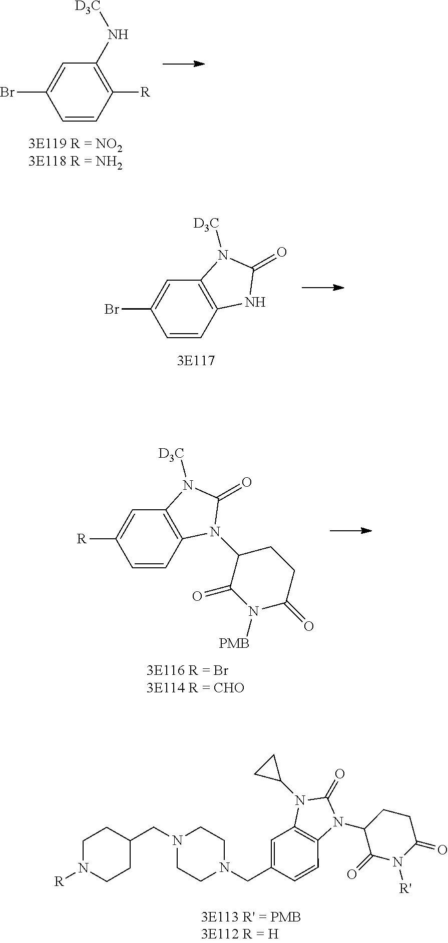

4-Bromo-2-fluoro-1-nitrobenzene (5.0 g), K₂CO₃ (9.4 g), and H₂NCD₃ (8.0 g, HCl salt) were stirred ON in MeOH, and diluted with water to precipitate Int. 3E119 5-bromo-2-nitro-N-(trideuteriomethyl)aniline (5.0 g). This material, NH₄Cl (10.9 g), and iron powder (5.7 g) were stirred 3 h in EtOH/water (2:1; 60 mL) and filtered. The OL (water/EtOAc) was dried and concentrated to give Int. 3E118 4-bromo-N2-(trideuterio-methyl)-benzene-1,2-diamine (4.0 g). 2.0 g of this material and CDI (1.1 g) were stirred ON in ACN (20 mL) at 0° C. to 80° C., concentrated, and triturated in Et₂O to give Int. 3E117 5-bromo-3-(trideuteriomethyl)-1H-benzimidazol-2-one (1.7 g). 1.0 g of this material and KOtBu (0.69 g) were stirred 0.5 h in THF (20 mL) at 0° C. Int. 3E3 (3.3 g) was added and stirring continued 4 h at 0° C. The OL (water/EtOAc) was dried, concentrated, and purified by FC (pentane/EtOAc 1:1 to 2:3) to give Int. 3E116 3-[5-bromo-2-oxo-3-(trideuterio-methyl)-benzimidazol-1-yl]-1-[(4-methoxyphenyl)-methyl]piperidine-2,6-dione (1.3 g). 0.3 g of this material, tert-butyl acrylate (0.27 mL), Pd(OAc)₂ (35 mg), and P(o-tol)₃ (40 mg) were degassed in DMF/Et₃N (18.5:1, 5.3 mL) and stirred ON at 110° C. The OL (EtOAc/water) was washed with brine, dried, concentrated, and triturated in pentane to give Int. 3E115 tert-butyl (E)-3-[1-[1-[(4-methoxyphenyl)-methyl]-2,6-dioxo-3-piperidyl]-2-oxo-3-(trideuteriomethyl)-benzimidazol-5-yl]prop-2-enoate (0.3 g). This material, NaIO₄ (0.31 g), and OsO₄ (15 mg) were stirred ON in THF/water (3:1; 16 mL). The OL (sat. aq. Na₂S₂O₃ and sat. aq. NaHCO₃/EtOAc) was washed with brine, dried, concentrated, and purified by FC (pentane/EtOAc 2:3 to 3:7) to give Int. 3E114 1-[1-[(4-methoxyphenyl)methyl]-2,6-dioxo-3-piperidyl]-2-oxo-3-(trideuteriomethyl)-benzimidazole-5-carbaldehyde (0.16 g). Ints. 3E5/3E114 (0.17 g/0.12 g) were stirred ON in DCE/DIPEA (26.7:1, 4.2 mL) at 0° C. to RT. STAB (0.25 g) was added and stirring continued ON at 0° C. to RT. The OL (water/EtOAc) was washed with brine, dried, concentrated, and purified by FC (DCM/MeOH 9:1) to give Int. 3E113 tert-butyl 4-[[4-[[1-[1-[(4-methoxyphenyl)methyl]-2,6-dioxo-3-piperidyl]-2-oxo-3-(trideuteriomethyl)-benzimidazol-5-yl]methyl]piperazin-1-yl]methyl]piperidine-1-carboxylate (0.15 g). Int. 3E113 (0.18 g) was stirred 1 h in TFA/TfOH (1.6:2; 3.6 mL) at 80° C., concentrated, and triturated in Et₂O to give Int. 3E112 3-(3-(methyl-d₃)-2-oxo-5-((4-(piperidin-4-ylmethyl)piperazin-1-yl)methyl)-2,3-dihydro-1H-benzo[d]imidazol-1-yl)piperidine-2,6-dione (0.14 g).

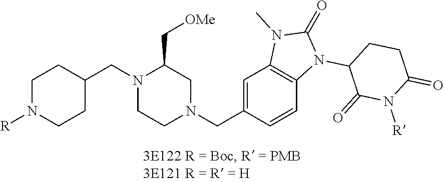

Ints. 3E3/3E123 (31 mg/50 mg) were stirred 6 h in DCE/DIPEA (12.5:1, 1.1 mL). STAB (64 mg) was added and stirring continued ON. The OL (water/DCM) was dried and concentrated. The residue was combined with another batch prepared similarly and purified by FC (hexane/EtOAc 1:4) to give Int. 3E122 tert-butyl 4-[[(2R)-2-(methoxy-methyl)-4-[[1-[1-[(4-methoxy-phenyl)methyl]-2,6-dioxo-3-piperidyl]-3-methyl-2-oxo-benz-imidazol-5-yl]methyl]piperazin-1-yl]methyl]-piperidine-1-carboxylate (30 mg). This material (20 mg) was stirred 1 h in TFA/TfOH (10:1; 0.22 mL) at 80° C. and concentrated to give Int. 3E121 3-(5-(((R)-3-(methoxymethyl)-4-(piperidin-4-ylmethyl)piperazin-1-yl)methyl)-3-methyl-2-oxo-2,3-dihydro-1H-benzo[d]-imidazol-1-yl)piperidine-2,6-dione (15 mg, TfOH salt).

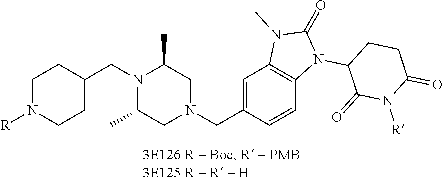

Ints. 3E3/3E127 (0.13 g/0.1 g) were stirred ON in DCE/DIPEA (14.7:1, 5.3 mL). STAB (0.27 g) was added and stirring continued ON. The OL (water/DCM/MeOH (9:1)) was washed with brine, dried, and concentrated to give Int. 3E126 tert-butyl 4-[[(2S,6S)-4-[[1-[1-[(4-methoxyphenyl)methyl]-2,6-dioxo-3-piperidyl]-3-methyl-2-oxo-benzimidazol-5-yl]-methyl]-2,6-dimethyl-piperazin-1-yl]methyl]-piperidine-1-carboxylate (0.15 g). This material was stirred 4 h in TFA/TfOH (5:2; 1.2 mL) at 80° C., concentrated, and triturated in Et₂O to give Int. 3E125 3-(5-(((3S,5S)-3,5-dimethyl-4-(piperidin-4-ylmethyl)-piperazin-1-yl)methyl)-3-methyl-2-oxo-2,3-dihydro-1H-benzo[d]imidazol-1-yl)-piperidine-2,6-dione (0.1 g, TfOH salt).

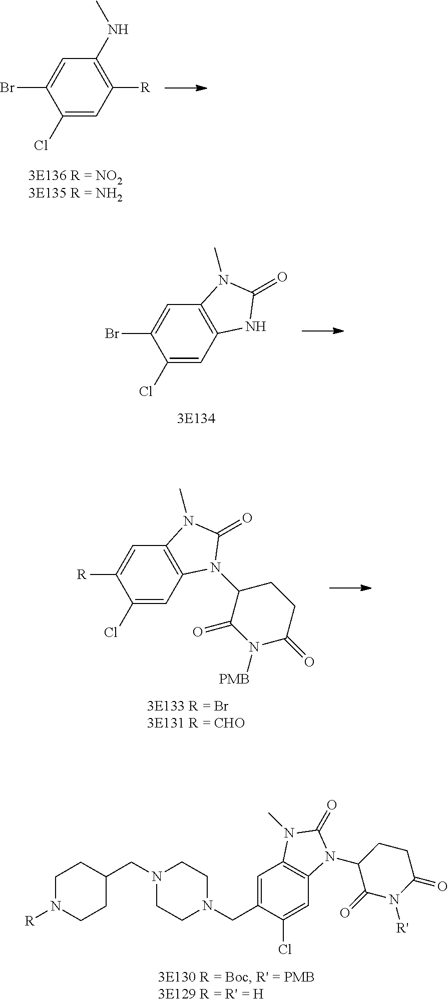

1-Bromo-2-chloro-5-fluoro-4-nitro-benzene (5.0 g) was stirred 4 h in 2M H₂NMe in THF (19.6 mL) at 0° C. to RT, concentrated, and triturated in pentane to give Int. 3E136 5-bromo-4-chloro-N-methyl-2-nitro-aniline (4.5 g). This material, NH₄Cl (9.1 g), and Iron powder (5.5 g) were stirred 3 h in EtOH/water (3.5:1, 45 mL) at 70-80° C. and filtered. The OL (water/EtOAc) was dried and concentrated to afford Int. 3E135 4-bromo-5-chloro-N2-methyl-benzene-1,2-diamine (4.0 g). 1.5 g of this material and CDI (2.1 g) were stirred ON in ACN (15 mL) at 80° C. The mixture was diluted with water to precipitate Int. 3E134 5-bromo-6-chloro-3-methyl-1H-benzimidazol-2-one (1.4 g). This material and KOtBu (1.2 g) were stirred 1 h in THF (30 mL) at 0° C. Int. 3E3 (3.1 g) was added and stirring continued 6 h at 0° C. The OL (water/EtOAc) was dried, concentrated, and purified by FC (pentane/EtOAc 1:1 to 2:3) to give Int. 3E133 3-(5-bromo-6-chloro-3-methyl-2-oxo-benz-imidazol-1-yl)-1-[(4-methoxyphenyl)methyl]-piperidine-2,6-dione (1.3 g). Int. 3E133 (2.0 g), tert-butyl acrylate (1.6 g), Pd(OAc)₂ (0.22 g), and P(o-tol)₃ (0.25 g) were degassed in DMF/Et₃N (11.8:1, 22 mL) and stirred ON at 110° C. The OL (water/EtOAc) was washed with brine, dried, concentrated, and purified by FC (pentane/EtOAc 7:3) to give Int. 3E132 tert-butyl (E)-3-(6-chloro-1-(1-(4-methoxy-benzyl)-2,6-dioxo-piperidin-3-yl)-3-methyl-2-oxo-2,3-dihydro-1H-benzo[d]imidazol-5-yl)acrylate (1.6 g). This material, NaIO₄ (1.9 g), and OsO₄ (0.15 g) were stirred ON in THF/water (1:1; 20 mL). The OL (sat. aq. Na₂S₂O₃/sat. aq. NaHCO₃/EtOAc) was washed with brine, dried, concentrated, and purified by FC (pentane/EtOAc 3:7) to give Int. 3E131 6-chloro-1-(1-(4-methoxybenzyl)-2,6-dioxopiperidin-3-yl)-3-methyl-2-oxo-2,3-dihydro-1H-benzo[d]imidazole-5-carbaldehyde (1.1 g). Ints. 3E5/3E131 (0.1 g/0.1 g) were stirred ON in DCE/DIPEA (16.7:1, 2.1 mL). STAB (0.1 g) was added and stirring continued ON at 0° C. to RT. The OL (water/DCM) was dried, concentrated, and purified by FC (DCM/MeOH 95:5) to give Int. 3E130 tert-butyl 4-[[4-[[6-chloro-1-[1-[(4-methoxyphenyl)-methyl]-2,6-dioxo-3-piperidyl]-3-methyl-2-oxo-benzimidazol-5-yl]-methyl]piperazin-1-yl]-methyl]piperidine-1-carboxylate (trideuterio-methyl)-benzimidazol-5-yl]methyl]piperazin-1-yl]-methyl]piperidine-1-carboxylate (0.1 g). Int. 3E130 (0.26 g) was stirred in TFA/TfOH (1:0.13; 2.26 mL) at 80° C. and concentrated to give Int. 3E129 3-(6-chloro-3-methyl-2-oxo-5-((4-(piperidin-4-ylmethyl)piperazin-1-yl)-methyl)-2,3-dihydro-1H-benzo[d]imidazol-1-yl)piperidine-2,6-dione (0.3 g, TfOH salt).

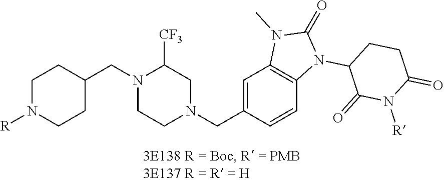

Ints. 3E3/3E139 (0.30 g/0.28 g) were stirred ON in MeOH (5 mL) and 1M ZnCl₂ in THF (1.5 mL). STAB was added and stirring continued ON. The OL (DCM/MeOH (9:1)/water) was dried, concentrated, and purified by FC (DCM/MeOH 9:1 to give Int. 3E138 tert-butyl 4-[[4-[[1-[1-[(4-methoxyphenyl)-methyl]-2,6-dioxo-3-piperidyl]-3-methyl-2-oxo-benz-imidazol-5-yl]methyl]-2-(trifluoromethyl)piperazin-1-yl]methyl]piperidine-1-carboxylate (0.3 g). A solution of this material (0.2 g) in TFA/TfOH (10:1; 3.3 mL) was stirred 5 h at 80° C. and concentrated to give Int. 3E137 3-(3-methyl-2-oxo-5-((4-(piperidin-4-yl-methyl)-3-(trifluoromethyl)-piperazin-1-yl)methyl)-2,3-dihydro-1H-benzo[d]imidazol-1-yl)piperidine-2,6-dione (0.1 g, TfOH salt).

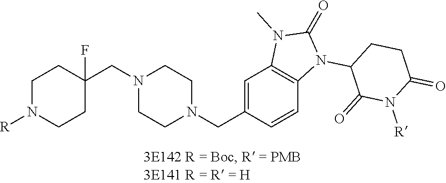

Ints. 3E3/3E143 (1.0 g/0.96 g) were stirred 6 h in DCE/DIPEA (8.8:1, 16.7 mL). STAB (1.0 g) was added and stirring continued ON at 0° C. to RT. The OL (DCM/water) was dried and concentrated. The residue was combined with another batch prepared similarly and purified by FC (pentane/EtOAc 1:4 to 1:9) to give Int. 3E142 tert-butyl 4-fluoro-4-[[4-[[1-[1-[(4-methoxy-phenyl)methyl]-2,6-dioxo-3-piperidyl]-3-methyl-2-oxo-benzimidazol-5-yl]methyl]piperazin-1-yl]methyl]-piperidine-1-carboxylate (1.12 g). 0.3 g of this material was stirred 4 h in TFA/TfOH (10:1; 5.5 mL) at 60° C. and concentrated to give Int. 3E141 3-(5-((4-((4-fluoro-piperidin-4-yl)methyl)piperazin-1-yl)methyl)-3-methyl-2-oxo-2,3-dihydro-1H-benzo[d]imidazol-1-yl)-piperidine-2,6-dione (0.20 g, TfOH salt).

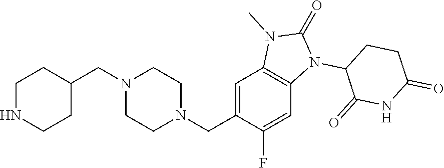 [SMILES: Cn1c(=O)n(C2CCC(=O)NC2=O)c2cc(F)c(CN3CCN(CC4CCNCC4)CC3)cc21]

Int. 3E60 (0.25 g) was stirred 3 h in TFA/TfOH (4:0.3; 4.3 mL) at 80° C. and concentrated to give Int. 3E145 3-(6-fluoro-3-methyl-2-oxo-5-((4-(piperidin-4-ylmethyl)piperazin-1-yl)methyl)-2,3-dihydro-1H-benzo[d]imidazol-1-yl)piperidine-2,6-dione (0.20 g, TfOH salt).

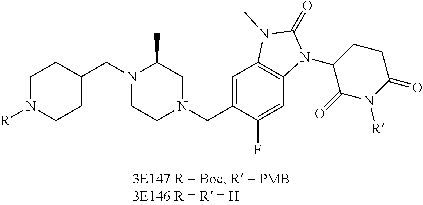

Ints. 3E61/3E74 (0.70 g/0.73 g) were stirred in DCE/DMSO/DIPEA (5:1:0.75; 13.5 mL). STAB (0.7 g) was added and stirring continued 48 h at 0° C. to RT. The OL (water/DCM) was dried, concentrated, and purified by FC (DCM/MeOH 1:0 to 95:5) to give Int. 3E147 tert-butyl 4-(((2S)-4-((6-fluoro-1-(1-(4-methoxy-benzyl)-2,6-dioxo-piperidin-3-yl)-3-methyl-2-oxo-2,3-dihydro-1H-benzo[d]imidazol-5-yl)methyl)-2-methyl-piperazin-1-yl)methyl)piperidine-1-carboxylate (0.75 g). This material was stirred 3 h in TFA/TfOH (10:1; 11 mL) at 80° C. and concentrated to give Int. 3E146 3-(6-fluoro-3-methyl-5-(((S)-3-methyl-4-(piperidin-4-ylmethyl)-piperazin-1-yl)methyl)-2-oxo-2,3-dihydro-1H-benzo[d]imidazol-1-yl)piperidine-2,6-dione (0.7 g, TfOH salt).

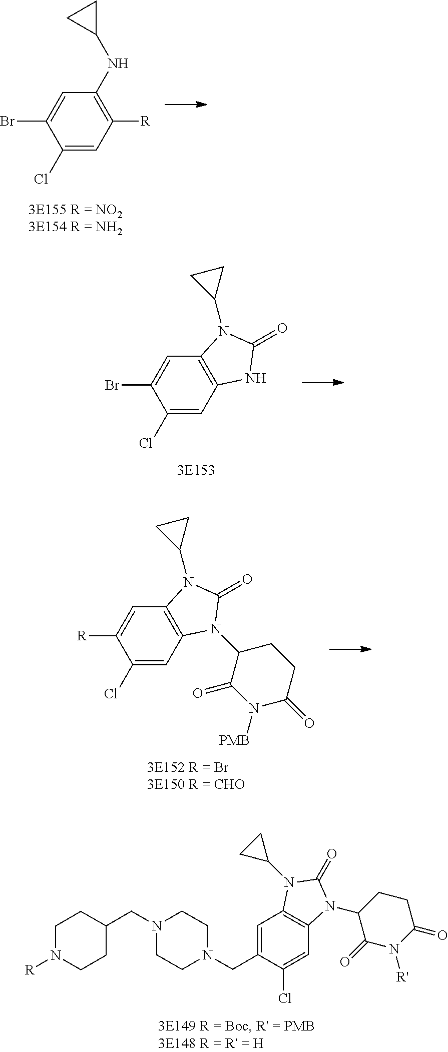

1-Bromo-2,5-difluoro-4-nitrobenzene (7.0 g) and cyclo-propyl amine (3.4 g) were stirred ON in THF (70 mL). The OL (water/EtOAc) was dried and concentrated to give Int. 3E155 5-bromo-N-cyclo-propyl-4-fluoro-2-nitro-aniline (8.0 g). This material, NH₄Cl (13.5 g), and Iron powder (7.1 g) were stirred 3 h in EtOH/water (1:1; 70 mL) at 70-80° C. and filtered. The OL (water/EtOAc) was dried and concentrated to give Int. 3E154 4-bromo-N2-cyclo-propyl-5-fluoro-benzene-1,2-diamine (5.0 g). Int. 3E154 (6.0 g) and CDI (6.0 g) were stirred ON in ACN (60 mL) at 80° C., and diluted with water to precipitate Int. 3E153 5-bromo-3-cyclo-propyl-6-fluoro-1H-benzimidazol-2-one (4.2 g). This material and KOtBu (2.8 g) were stirred 0.5 h in THF (42 mL) at 0° C. Int. 3E3 (9.5 g in 10 mL THF) was added and stirring continued 3 h at 0° C. The OL (EtOAc/water) was washed with brine, dried, and concentrated to give Int. 3E152 3-(5-bromo-3-cyclo-propyl-6-fluoro-2-oxo-benzimidazol-1-yl)-1-[(4-methoxyphenyl)methyl]-piperidine-2,6-dione (4.2 g). This material, tert-butyl acrylate (3.1 mL), Pd(OAc)₂ (0.45 g), and P(o-tol)₃ (0.51 g) were degassed in DMF/Et₃N (5.9:1, 23.4 mL) and stirred ON at 110° C. The OL (water/EtOAc) was washed with brine, dried, concentrated, and purified by FC (hexane/EtOAc 3:2) to give Int. 3E151 tert-butyl (E)-3-[3-cyclo-propyl-6-fluoro-1-[1-[(4-methoxyphenyl)-methyl]-2,6-dioxo-3-piperidyl]-2-oxo-benzimidazol-5-yl]prop-2-enoate (4.3 g). This material, NaIO₄ (4.2 g), and OsO₄ (0.4 g) were stirred ON in THF/water (3:1; 44 mL). The OL (sat. aq. Na₂S₂O₃ and sat. aq. NaHCO₃/EtOAc) was washed with brine, dried, and concentrated to give Int. 3E150 3-cyclo-propyl-6-fluoro-1-[1-[(4-methoxyphenyl)methyl]-2,6-dioxo-3-piperidyl]-2-oxo-benzimidazole-5-carbaldehyde (2.4 g). Ints. 3E5/3E150 (0.59 g/0.47 g) were stirred ON in DCE/DIPEA (9.3:1, 5.5 mL). STAB (0.44 g) was added and stirring continued ON at 0° C. to RT. The OL (10% aq. NH₄Cl/DCM/MeOH (9:1) was washed with brine, dried, concentrated, and purified by FC (hexane/EtOAc 1:4) to give Int. 3E149 tert-butyl 4-[[4-[[3-cyclo-propyl-6-fluoro-1-[1-[(4-methoxyphenyl)methyl]-2,6-dioxo-3-piperidyl]-2-oxo-benzimidazol-5-yl]methyl]-piperazin-1-yl]methyl]-piperidine-1-carboxylate (0.23 g). 0.18 g of this material was stirred 1 h in TFA/TfOH (10:1; 3.3 mL) at 0° C. and 1 h at 70° C. and concentrated to give Int. 3E148 3-(3-cyclo-propyl-6-fluoro-2-oxo-5-((4-(piperidin-4-ylmethyl)piperazin-1-yl)methyl)-2,3-dihydro-1H-benzo[d]imidazol-1-yl)piperidine-2,6-dione (0.27 g, TfOH salt).

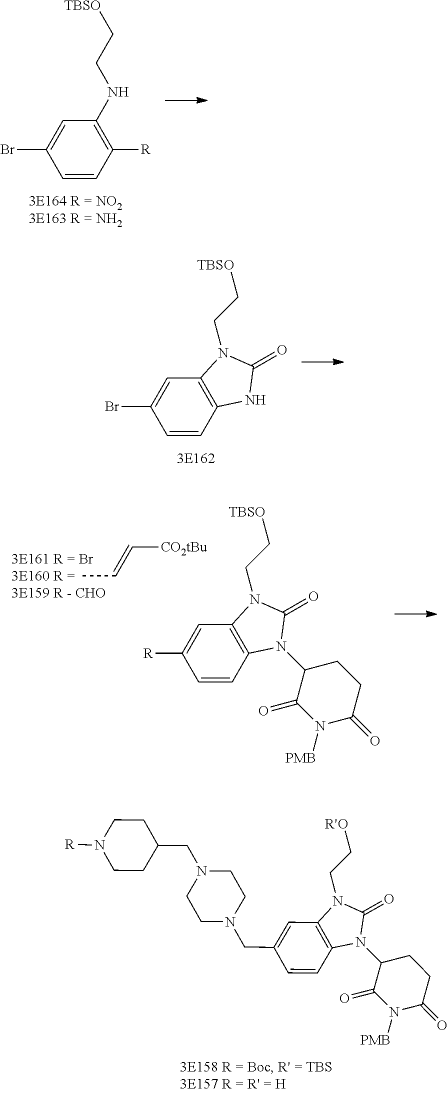

1-Bromo-3-fluoro-4-nitrobenzene (1.0 g), 2-[tert-butyl(dimethyl)silyl]oxyethanamine (0.96 g), and K₂CO₃ (1.3 g) were stirred ON in THF (5 mL) at 80° C. The OL (EtOAc/water) was dried and concentrated and combined with another batch prepared similarly from 1-bromo-3-fluoro-4-nitrobenzene (4.8 g) to give Int. 3E164 5-bromo-N-[2-[tert-butyl(dimethyl)silyl]oxyethyl]-2-nitro-aniline (8.0 g). This material, NH₄Cl (11.3 g), and iron powder (5.8 g) were stirred ON in EtOH/water (1:1; 60 mL) at 70° C. and filtered. The OL (water/EtOAc) was dried and concentrated to give Int. 3E163 4-bromo-N2-[2-[tert-butyl(dimethyl)silyl]-oxyethyl]-benzene-1,2-diamine (6.0 g). Int. 3E163 (5.5 g) and CDI (5.2 g) were stirred ON in ACN (30 mL) at 80° C., concentrated, and triturated in water to give Int. 3E162 5-bromo-3-[2-[tert-butyl-(dimethyl)-silyl]-oxyethyl]-1H-benz-imidazol-2-one (5.0 g). Int. 3E162 (4.1 g) and KOtBu (1.9 g) were stirred 0.5 h in THF (10 mL) at 0° C. Int. 3E3 (8.4 g) was added and stirring continued 4 h at 0° C. The OL (water/EtOAc) was washed with brine, dried, and concentrated to give Int. 3E161 3-[5-bromo-3-[2-[tert-butyl-(dimethyl)silyl]oxy-ethyl]-2-oxo-benzimidazol-1-yl]-1-[(4-methoxyphenyl)-methyl]piperidine-2,6-dione (2.0 g). This material, tert-butyl acrylate (1.7 g), Pd(OAc)₂ (0.23 g), and P(o-tol)₃ (0.21 g) were degassed in DMF/Et₃N (5.9:1, 5.4 mL) and stirred ON at 110° C. The OL (water/EtOAc) was washed with brine, dried, concentrated, and purified by FC (hexane/EtOAc 7:3 to 3:2) to give Int. 3E160 tert-butyl (E)-3-[3-[2-[tert-butyl(dimethyl)silyl]-oxyethyl]-1-[1-[(4-methoxyphenyl)methyl]-2,6-dioxo-3-piperidyl]-2-oxo-benzimidazol-5-yl]prop-2-enoate (1.5 g). This material, NaIO₄ (1.5 g), and OsO₄ (6 mg) were stirred ON in THF/water (2:1; 15 mL) at 0° C. to RT. The OL (sat. aq. Na₂S₂O₃ and sat. aq. NaHCO₃/EtOAc) was washed with brine, dried, concentrated, and purified by FC (pentane/EtOAc 3:7 to 1:4) to give Int. 3E159 3-[2-[tert-butyl(dimethyl)silyl]oxyethyl]-1-[1-[(4-methoxy-phenyl)methyl]-2,6-dioxo-3-piperidyl]-2-oxo-benzimidazole-5-carbaldehyde (0.8 g). Ints. 3E5/3E159 (0.53 g/0.8 g) were stirred ON in DCE/DIPEA (19:1, 26 mL). STAB (0.77 g) was added and stirring continued ON at 0° C. to RT. The OL (water/DCM) was concentrated and purified by FC (DCM/MeOH 95:5 to 9:1) to give Int. 3E158 tert-butyl 4-[[4-[[3-[2-[tert-butyl-(dimethyl)silyl]oxyethyl]-1-[1-[(4-methoxyphenyl)-methyl]-2,6-dioxo-3-piperidyl]-2-oxo-benzimidazol-5-yl]methyl]piperazin-1-yl]methyl]-piperidine-1-carboxylate (0.24 g). This material (0.16 g) was stirred 4 h in 1.8M HCl in dioxane (9 mL) at 0° C. to RT, concentrated, and triturated in Et₂O to give Int. 3E157 3-[3-(2-hydroxyethyl)-2-oxo-5-[[4-(4-piperidylmethyl)-piperazin-1-yl]methyl]benzimidazol-1-yl]-1-[(4-methoxy-phenyl)methyl]-piperidine-2,6-dione (0.13 g, HCl salt).

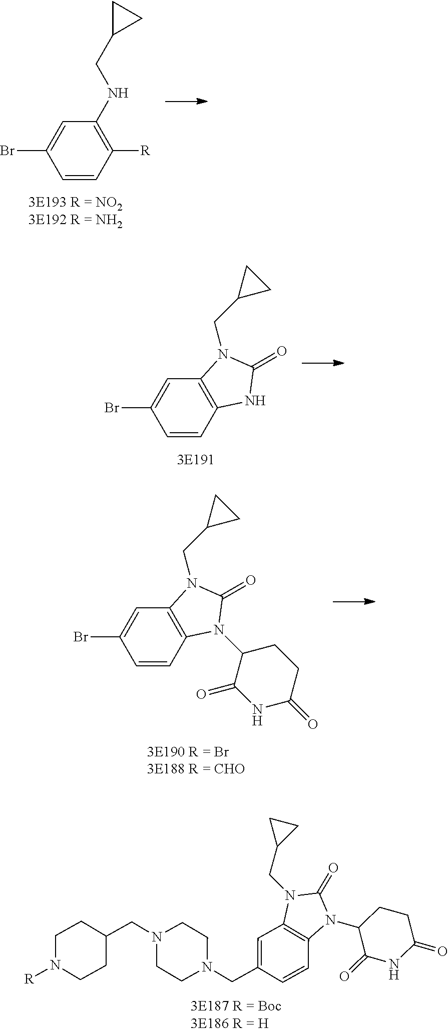

cyclo-Propylmethanamine (4.5 g) and 4-bromo-2-fluoro-1-nitrobenzene (7.0 g) were stirred in THF (50 mL) at 0° C. The OL (water/EtOAc) was washed with brine, dried, and concentrated to give Int. 3E193 5-bromo-N-(cyclo-propylmethyl)-2-nitro-aniline (8.0 g). This material, NH₄Cl (15.0 g), and iron powder (7.8 g) were stirred 3 h in EtOH/water (2:1; 60 mL) at 80° C. and filtered. The OL (water/EtOAc) was dried and concentrated to give Int. 3E194 4-bromo-N2-(cyclo-propylmethyl)benzene-1,2-diamine (6.5 g). Int. 3E194 (5.0 g) and CDI (4.3 g) were stirred 3 h in ACN (30 mL) at 80° C. The OL (water/EtOAc) was washed with brine, dried, concentrated, and triturated in Et₂O to give Int. 3E191 5-bromo-3-(cyclo-propylmethyl)-1H-benzimidazol-2-one (4.7 g). Int. 3E191 (2.5 g) and NaH (60% oil disp., 2.6 g) were stirred 0.5 h in THF (25 mL). 3-Bromo-piperidine-2,6-dione (6.3 g) was added and stirring continued ON at 65° C. The OL (water/EtOAc) was washed with brine, dried, concentrated, and purified by FC (pentane/EtOAc 7:3 to 1:1) to Int. 3E190 3-[5-bromo-3-(cyclo-propylmethyl)-2-oxo-benzimidazol-1-yl]piperidine-2,6-dione (1.5 g). This material, tert-butyl acrylate (2.0 g), Pd(OAc)₂ (0.27 g), and P(o-tol)₃ (0.24 g) were degassed in DMF/Et₃N (4.5:1, 12.2 mL) and stirred ON at 120° C. The OL (water/EtOAc) was washed with brine, dried, concentrated, and purified by FC (pentane/EtOAc 7:3 to 3:2) to give Int. 3E189 tert-butyl (E)-3-[3-(cyclo-propyl-methyl)-1-(2,6-dioxo-3-piperidyl)-2-oxo-benzimidazol-5-yl]prop-2-enoate (1.0 g). Int. 3E189 (1.4 g), NaIO₄ (1.8 g), and OsO₄ (17 mg) were stirred ON in THF/water (5:1; 18 mL) at 0° C. to RT. The OL (sat. aq. Na₂S₂O₃ and sat. aq. NaHCO₃/EtOAc) was washed with brine, dried, concentrated, and purified by FC (pentane/EtOAc 4:1) to give Int. 3E188 3-(cyclo-propylmethyl)-1-(2,6-dioxo-3-piperidyl)-2-oxo-benzimidazole-5-carbaldehyde (0.7 g). Ints. 3E5/3E188 (0.35 g/0.25 g) were stirred ON in DCE/DIPEA (10:1, 5.5 mL). STAB (0.23 g) was added and stirring continued ON at 0° C. to RT. The OL (water/DCM) was concentrated and purified by FC (DCM/MeOH 95:5 to 9:1) to give Int. 3E187 tert-butyl 4-[[4-[[3-(cyclo-propylmethyl)-1-(2,6-dioxo-3-piperidyl)-2-oxo-benzimidazol-5-yl]methyl]piperazin-1-yl]methyl]-piperidine-1-carboxylate (0.14 g). Int. 3E187 (0.13 g) was stirred 5 h in DCM/4M HCl in dioxane (5:1.3; 6.3 mL) at 0° C. to RT and concentrated to give Int. 3E186 3-(3-(cyclo-propylmethyl)-2-oxo-5-((4-(piperidin-4-ylmethyl)-piperazin-1-yl)methyl)-2,3-dihydro-1H-benzo[d]imidazol-1-yl)piperidine-2,6-dione (0.12 g, HCl salt).

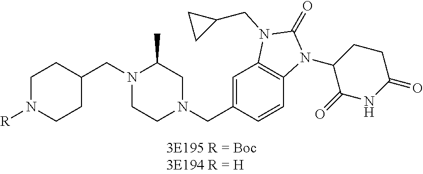

Ints. 3E74/3E188 (0.27 g/0.20 g) were stirred ON in DCE/DIPEA (9.4:1, 2.5 mL). STAB (0.39 g) was added and stirring continued 36 h at 0° C. to RT. The OL (water/DCM) was washed with brine, dried, concentrated, and purified by FC (DCM/MeOH 95:5 to 9:1) to give Int. 3E195 tert-butyl 4-(((2S)-4-((3-(cyclo-propylmethyl)-1-(2,6-dioxopiperidin-3-yl)-2-oxo-2,3-dihydro-1H-benzo[d]-imidazol-5-yl)methyl)-2-methyl-piperazin-1-yl)-methyl)-piperidine-1-carboxylate (0.20 g), which was stirred 3 h in 0.5M HCl in dioxane (2.3 mL) at 0° C. to RT and concentrated to give Int. 3E194 3-(3-(cyclo-propylmethyl)-5-(((S)-3-methyl-4-(piperidin-4-yl-methyl)-piperazin-1-yl)methyl)-2-oxo-2,3-dihydro-1H-benzo[d]imidazol-1-yl)piperidine-2,6-dione (0.15 g, HCl salt).

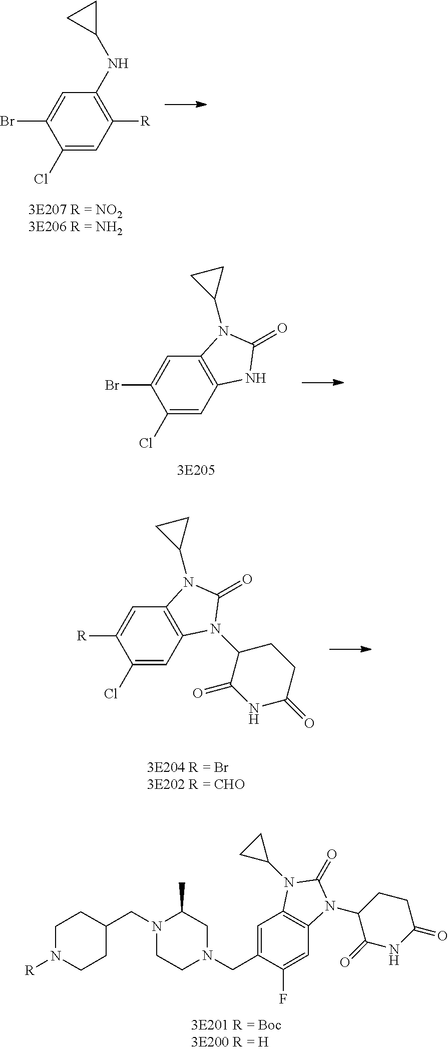

cyclo-Propylmethanamine (3.4 g) and 1-bromo-2,5-difluoro-4-nitrobenzene (7.0 g) were stirred ON in THF (70 mL). The OL (water/EtOAc) was dried and concentrated to give Int. 3E207 5-bromo-N-cyclo-propyl-4-fluoro-2-nitro-aniline (8.0 g). 7.0 g of this material, NH₄Cl (13.6 g), and iron powder (7.1 g) were stirred 3 h in EtOH/water (1:1; 70 mL) at 70-80° C. and filtered. The OL (water/EtOAc) was dried and concentrated to give Int. 3E206 4-bromo-N2-cyclo-propyl-5-fluoro-benzene-1,2-diamine (5.0 g). Int. 3E206 (6.0 g) and CDI (5.9 g) were stirred ON in ACN (60 mL) at 80° C., concentrated, and triturated in water to give Int. 3E205 5-bromo-3-cyclo-propyl-6-fluoro-1H-benzimidazol-2-one (4.2 g). Int. 3E205 (6.0 g) and KOtBu (4.0 g) were stirred 0.5 h in THF (60 mL). Int. 3A8 (13.5 g) was added and stirring continued 3 h at 0° C. The OL (aq. NH₄Cl/DCM/MeOH (9:1)) was washed with brine, dried, and concentrated to give Int. 3E204 3-(5-bromo-3-cyclo-propyl-6-fluoro-2-oxo-benzimidazol-1-yl)-1-[(4-methoxy-phenyl)methyl]-piperidine-2,6-dione (7.1 g). 6.1 g of this material, tert-butyl acrylate (5.3 mL), Pd(OAc)₂ (0.82 g), and P(o-tol)₃ (0.74 g) were degassed in DMF/Et₃N (10:1, 55 mL) and stirred ON at 110° C. The OL (water/EtOAc) was washed with brine, dried, concentrated, and purified by FC (pentane/EtOAc 3:2) to give Int. 3E203 tert-butyl (E)-3-[3-cyclo-propyl-6-fluoro-1-[1-[(4-methoxyphenyl)methyl]-2,6-dioxo-3-piperidyl]-2-oxo-benzimidazol-5-yl]prop-2-enoate (6.0 g). This material, NaIO₄ (5.8 g), and OsO₄ (0.55 mg) were stirred ON in THF/water (3:1; 60 mL). The OL (water/EtOAc) was washed with brine, dried, concentrated, and purified by FC (hexane/EtOAc 3:2) to give Int. 3E202 3-cyclo-propyl-6-fluoro-1-[1-[(4-methoxyphenyl)-methyl]-2,6-dioxo-3-piperidyl]-2-oxo-benzimidazole-5-carbaldehyde (3.1 g). Ints. 3E5/3E202 (0.99 g/1.5 g) were stirred ON in DCE/DIPEA (5.6:1, 11.8 mL). STAB (1.4 g in DMSO (5 mL)) was added at 0° C. and stirring continued ON at 0° C. to 60° C. The OL (aq. NH₄Cl/EtOAc) was concentrated and purified by FC (hexane/EtOAc 1:4) to give Int. 3E201 tert-butyl 4-[[(2S)-4-[[3-cyclo-propyl-6-fluoro-1-[1-[(4-methoxyphenyl)-methyl]-2,6-dioxo-3-piperidyl]-2-oxo-benz-imidazol-5-yl]methyl]-2-methyl-piperazin-1-yl]methyl]piperidine-1-carboxylate (1.2 g). This material was stirred 2 h in TFA/TfOH (9:1; 10 mL) at 70° C. and concentrated to give Int. 3E200 3-(3-cyclo-propyl-6-fluoro-5-(((S)-3-methyl-4-(piperidin-4-ylmethyl)piperazin-1-yl)methyl)-2-oxo-2,3-dihydro-1H-benzo[d]imidazol-1-yl)piperidine-2,6-dione (1.5 g, TfOH salt).

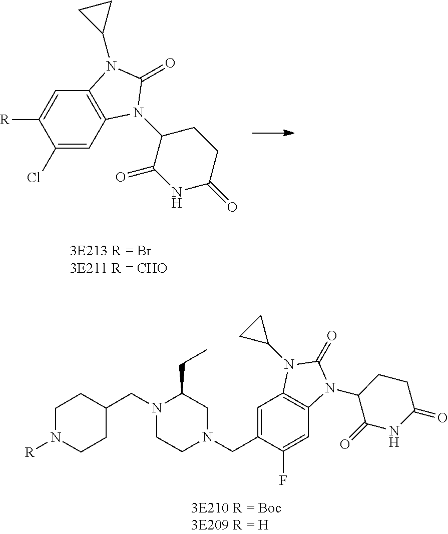

5-Bromo-3-cyclo-propyl-1H-benzimidazol-2-one (3.0 g) and NaH (60% oil disp. 7.9 g) were stirred 0.3 h in THF (70 mL) at 0° C. 3-Bromopiperidine-2,6-dione (19.0 g) was added and stirring continued ON at 0-60° C. The OL (water/EtOAc) was washed with brine, dried, concentrated, and triturated in hexane to give Int. 3E213 3-(5-bromo-3-cyclo-propyl-2-oxo-benzimidazol-1-yl)piperidine-2,6-dione (5.2 g). Int. 3E213 (6.1 g), tert-butyl acrylate (2.6 g), Pd(OAc)₂ (0.44 g), and P(o-tol)₃ (0.50 g) were degassed in DMF/Et₃N (17.6:1, 63.4 mL) and stirred ON at 110° C. and filtered. The OL (water/EtOAc). was washed with brine, dried, concentrated, and triturated in pentane to give Int. 3E212 tert-butyl (E)-3-[3-cyclo-propyl-1-(2,6-dioxo-3-piperidyl)-2-oxo-benz-imidazol-5-yl]prop-2-enoate (2.9 g). 1.4 g of this material, NaIO₄ (1.8 g), and OsO₄ (0.43 g) were stirred ON in THF/water (4.7:1, 17 mL). The OL (water/EtOAc) was washed with brine, dried, concentrated, and purified by FC (pentane/EtOAc 1:1 to 2:3) to give Int. 3E211 3-cyclo-propyl-1-(2,6-dioxo-3-piperidyl)-2-oxo-benzimidazole-5-carbaldehyde (0.80 g). Ints. 3E101/3E211 (1.4 g/0.55 g) and ZnCl₂ were stirred ON in DCE (6 mL). NaCNBH₃ (0.22 g) was added and stirring continued ON at 0° C. to RT. The OL (water/EtOAc) was washed with brine, dried, concentrated, and purified by FC (DCM/MeOH 1:0 to 4:1) to give Int. 3E210 tert-butyl 4-[[(2S)-4-[[3-cyclo-propyl-1-(2,6-dioxo-3-piperidyl)-2-oxo-benzimidazol-5-yl]methyl]-2-ethyl-piperazin-1-yl]methyl]-piperidine-1-carboxylate (0.80 g). A solution of 0.75 g of this material in DCM/4M HCl in dioxane (5:1, 6 mL) was stirred ON at 0° C. to RT and concentrated to give Int. 3E209 3-[3-cyclo-propyl-5-[[(3S)-3-ethyl-4-(4-piperidylmethyl)piperazin-1-yl]methyl]-2-oxo-benzimidazol-1-yl]-piperidine-2,6-dione (0.65 g, HCl salt).

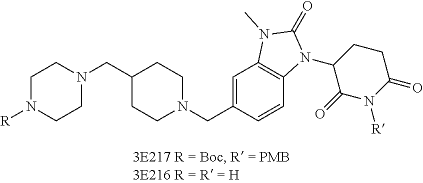

Ints. 3E3/3E218 (50 mg/42 mg) were stirred 0.25 h in DCE (2 mL) and a few drops of DMF. STAB (36 mg) was added and stirring continued 120 h. STAB (77 mg) and DIPEA (86 microL) were added and stirring continued 2 h at 70° C. The OL (water/DCM) was washed with brine, dried, concentrated, and purified by FC (DCM/MeOH 1:0 to 4:1) to give Int. 3E217 tert-butyl 4-[[1-[[1-[1-[(4-methoxyphenyl)methyl]-2,6-dioxo-3-piperidyl]-3-methyl-2-oxo-benzimidazol-4-yl]methyl]-4-piperidyl]-methyl]piperazine-1-carboxylate (33 mg). This material was stirred 4 h in TFA/TfOH (2:0.05; 2.05 mL) and ON at 50° C. and concentrated to give Int. 3E216 3-(3-methyl-2-oxo-5-((4-(piperazin-1-ylmethyl)-piperidin-1-yl)methyl)-2,3-dihydro-1H-benzo[d]-imidazol-1-yl)piperidine-2,6-dione (22 mg, TfOH salt).

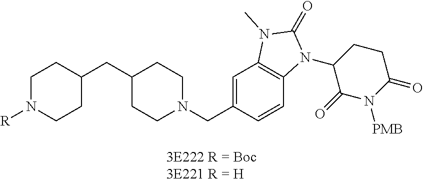

Int. 3E3 (50 mg) tert-butyl 4-(piperidin-4-ylmethyl)piperidine-1-carboxylate (47 mg, HCl salt) were stirred 0.3 h in DCE/AcOH/DIPEA (28.6:1:6.1, 2.5 mL) at 70° C. STAB (0.13 g) was added and stirring continued 3 h. The OL (water/DCM) was washed with brine, dried, concentrated, and purified by FC (DCM/MeOH 1:0 to 4:1) to give Int. 3E222 tert-butyl 4-[[1-[[1-[1-[(4-methoxyphenyl)methyl]-2,6-dioxo-3-piperidyl]-3-methyl-2-oxo-benz-imidazol-4-yl]methyl]-4-piperidyl]-methyl]piperazine-1-carboxylate (57 mg). A solution of this material in DCM/TFA (4:1; 2.5 mL) was stirred 0.5 h and concentrated to give Int. 3E221 3-[3-methyl-2-oxo-4-[[4-(4-piperidylmethyl)piperazin-1-yl]-methyl]benzimidazol-1-yl]piperidine-2,6-dione (58 mg, TFA salt).

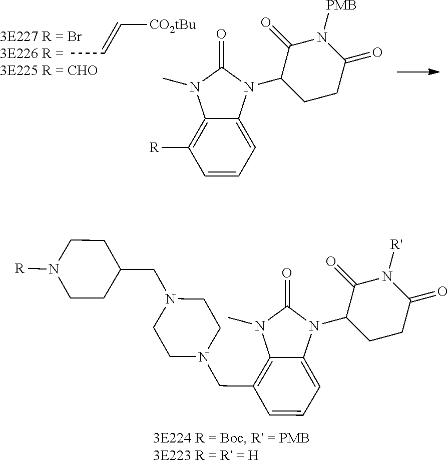

7-Bromo-1-methyl-1,3-dihydro-2H-benzo[d]imidazol-2-one (0.25 g) and KOtBu (0.16 g) were stirred 0.25 h in THF (3 mL) at 0° C. Int. 3A8 (0.67 g) was added and stirring continued 1 h. The residue after concentration was purified by FC (pentane/EtOAc 1:0 to 7:3) to give Int. 3E227 3-(4-bromo-3-methyl-2-oxo-benzimidazol-1-yl)-1-[(4-methoxy-phenyl)methyl]-piperidine-2,6-dione (0.15 g). Int. 3E227 (0.4 g), tert-butyl acrylate (0.32 mL), Pd(OAc)₂ (20 mg), and P(o-tol)₃ (53 mg) were degassed in DMF/Et₃N (16.6:1, 5.4 mL) and stirred ON at 120° C. The OL (water/EtOAc) was washed with brine, dried, concentrated, and purified by FC (heptane/EtOAc 1:1 to 0:1) to give Int. 3E226 tert-butyl (E)-3-[1-[1-[(4-methoxyphenyl)-methyl]-2,6-dioxo-3-piperidyl]-3-methyl-2-oxo-benzimidazol-4-yl]prop-2-enoate (0.44 g). This material and NaIO₄ (0.47 g) were stirred ON in THF/water (2:1; 3 mL) and 2.5% OsO₄ in tBuOH (0.11 mL). The OL (2MeTHF/sat. aq. NaHCO₃) was washed with 5% aq. Na₂S₂O₃ and brine, dried, concentrated, and triturated in DCM/heptane to give Int. 3E225 1-(1-(4-methoxy-benzyl)-2,6-dioxopiperidin-3-yl)-3-methyl-2-oxo-2,3-dihydro-1H-benzo[d]imidazole-4-carbaldehyde (0.34 g). Ints. 3E5/3E225 (42 mg/50 mg) were stirred 0.3 h in DCE/AcOH/DIPEA (28.6:1:6.1, 2.1 mL) at 70° C. STAB (0.13 g) was added and stirring continued 1 h at 70° C. The OL (water/DCM) was washed with brine, dried, concentrated, and purified by FC (DCM/MeOH 1:0 to 4:1) to give Int. 3E224 tert-butyl 4-[[1-[[1-[1-[(4-methoxyphenyl)-methyl]-2,6-dioxo-3-piperidyl]-3-methyl-2-oxo-benzimidazol-4-yl]-methyl]-4-piperidyl]-methyl]piperazine-1-carboxylate (74 mg). A solution of this material in TFA/TfOH (2:0.1; 2.1 mL) was stirred 4 h at 70° C. and ON at 50° C. and concentrated to give Int. 3E223 3-(3-methyl-2-oxo-4-((4-(piperidin-4-ylmethyl)-piperazin-1-yl)methyl)-2,3-dihydro-1H-benzo[d]imidazol-1-yl)piperidine-2,6-dione (50 mg, TfOH salt).

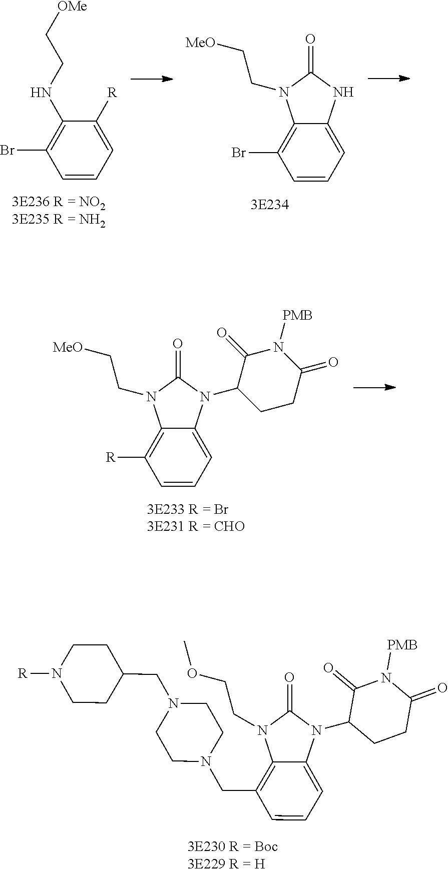

1-Bromo-2-fluoro-3-nitro-benzene (5.0 g) and 2-methoxyethan-1-amine (1.9 g) were stirred ON in MeOH (50 mL) at 0° C. to RT. The OL (water/EtOAc) was washed with brine, dried, and concentrated to give Int. 3E236 2-bromo-N-(2-methoxyethyl)-6-nitro-aniline (6.0 g). 3.8 g of this material and iron powder (3.9 g) were stirred 2 h in AcOH (38 mL) at 40° C. and filtered. The OL (aq. NaHCO₃/EtOAc) was washed with brine, dried, concentrated, and purified by FC (pentane/EtOAc 9:1 to 4:1) to give Int. 3E235 3-bromo-N2-(2-methoxyethyl)benzene-1,2-diamine (2.5 g). This material and CDI (2.5 g) were stirred ON in ACN (25 mL) at 80° C. and concentrated. The residue was triturated in water to give Int. 3E234 4-bromo-3-(2-methoxy-ethyl)-1H-benzimidazol-2-one (2.5 g). 0.7 g of this material and KOtBu (0.32 g) were stirred 0.5 h in THF (14 mL) at −10° C. to 0° C. Int. 3A8 (1.48 g) was added as a solution in THF (20 mL) at −10° C. and stirring was continued 1 h at 0° C. The OL (sat. aq. NH₄Cl/EtOAc) was washed with brine, dried, concentrated, and purified by FC (hexane/EtOAc 9:1 to 4:1) to give Int. 3E233 3-[4-bromo-3-(2-methoxyethyl)-2-oxo-benzimidazol-1-yl]-1-[(4-methoxy-phenyl)-methyl]-piperidine-2,6-dione (0.61 g). 0.4 g of this material, tert-butyl acrylate (0.29 mL), Pd(OAc)₂ (18 mg), and P(o-tol)₃ (48 mg) were degassed in DMF/Et₃N (16.7:1, 5.3 mL) and stirred ON at 120° C. The OL (water/EtOAc) was washed with brine, dried, concentrated, and purified by FC (heptane/EtOAc 1:0 to 0:1) to give Int. 3E232 tert-butyl (E)-3-(1-(1-(4-methoxybenzyl)-2,6-dioxo-piperidin-3-yl)-3-(2-methoxyethyl)-2-oxo-2,3-dihydro-1H-benzo[d]imidazol-4-yl)-acrylate (0.41 g). 0.39 g of this material and NaIO₄ (0.38 g), and 2.5% OsO₄ in tBuOH (89 microL) were stirred ON in THF/water (2:1; 9 mL). The OL (sat. aq. Na₂S₂O₃ and sat. aq. NaHCO₃/EtOAc) was washed with brine, dried, concentrated, and purified by FC (EtOAc/heptane 0:1 to 1:0) to give Int. 3E231 3-(2-methoxy-ethyl)-1-[1-[(4-methoxyphenyl)-methyl]-2,6-dioxo-3-piperidyl]-2-oxo-benz-imidazole-4-carbaldehyde (0.26 g). Ints. 3E5/3E231 (0.11 g/0.12 g) were stirred 0.5 h in DCE/DIPEA (87:1, 4.05) at 70° C. STAB (0.28 g) was added and stirring continued 4 h at 70° C. The OL (water/DCM) was washed with brine, dried, concentrated, and purified by FC (DCM/MeOH 1:0 to 4:1) to give Int. 3E230 tert-butyl 4-((4-((1-(1-(4-methoxybenzyl)-2,6-dioxopiperidin-3-yl)-3-(2-methoxyethyl)-2-oxo-2,3-dihydro-1H-benzo[d]-imidazol-4-yl)methyl)piperazin-1-yl)methyl)piperidine-1-carboxylate (0.15 g), which was stirred 1 h in DCM/TFA (3:2; 5 mL) and concentrated to give Int. 3E229 1-(4-methoxybenzyl)-3-(3-(2-methoxy-ethyl)-2-oxo-4-((4-(piperidin-4-ylmethyl)piperazin-1-yl)methyl)-2,3-dihydro-1H-benzo[d]-imidazol-1-yl)-piperidine-2,6-dione (0.11 g, TFA salt).

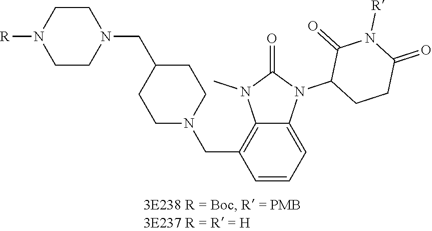

Ints. 3E225/3E218 (50 mg/42 mg) were stirred 0.3 h in DCE/AcOH/DIPEA (28.6:1:6.4, 2.0.5 mL) at 70° C. STAB (0.13 g) was added and stirring continued 1 h at 70° C. The OL (water/DCM) was washed with brine, dried, concentrated, and purified by FC (DCM/MeOH 1:0 to 4:1) to give Int. 3E238 tert-butyl 4-[[1-[[1-[1-[(4-methoxyphenyl)methyl]-2,6-dioxo-3-piperidyl]-3-methyl-2-oxo-benzimidazol-4-yl]methyl]-4-piperidyl]-methyl]piperazine-1-carboxylate (77 mg). This material was stirred 4 h in TFA/TfOH (2:0.1; 2.1 mL) at 70° C. and concentrated to give Int. 3E237 3-[3-methyl-2-oxo-4-[[4-(piperazin-1-ylmethyl)-1-piperidyl]methyl]-benzimidazol-1-yl]piperidine-2,6-dione (52 mg, TfOH salt).

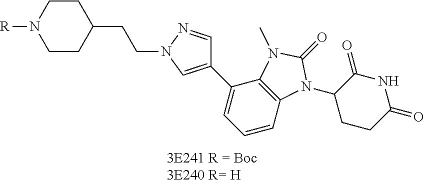

Ints. 3E242/3A6 (55 mg/35 mg), and PdDPPFCl₂-DCM (9 mg) were degassed in DMF/10% aq. Na₂CO₃ (5.6:1, 1.5 mL) and stirred 1.25 h at 100° C. The OL (water/EtOAc) was washed with brine, dried, concentrated, and purified by FC (heptane/EtOAc 3:2 to 0:1) to give Int. 3E241 tert-butyl 4-(2-(4-(1-(2,6-dioxopiperidin-3-yl)-3-methyl-2-oxo-2,3-dihydro-1H-benzo[d]imidazol-4-yl)-1H-pyrazol-1-yl)-ethyl)-piperidine-1-carboxylate (18 mg). A solution of Int. 3E241 (16 mg) in MeOH/4M HCl in dioxane (1:1; 0.4 mL) was stirred 0.75 h and concentrated to give Int. 3E240 3-(3-methyl-2-oxo-4-(1-(2-(piperidin-4-yl)-ethyl)-1H-pyrazol-4-yl)-2,3-dihydro-1H-benzo[d]imidazol-1-yl)-piperidine-2,6-dione (17 mg, HCl salt).

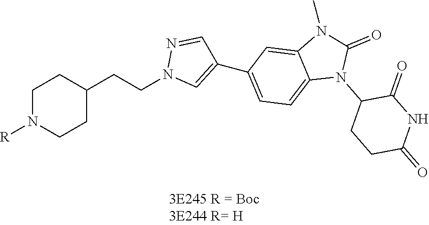

Ints. 3E242/3B3 (55 mg/35 mg), and PdDPPFCl₂-DCM (9 mg) were degassed in DMF/10% aq. Na₂CO₃ (5.7:1, 1.5 mL) and stirred 1.25 h at 100° C. The OL (water/EtOAc) was washed with brine, dried, concentrated, and purified by FC (heptane/EtOAc 3:2 to 0:1) to give Int. 3E245 tert-butyl 4-(2-(4-(1-(2,6-dioxopiperidin-3-yl)-3-methyl-2-oxo-2,3-dihydro-1H-benzo[d]imidazol-5-yl)-1H-pyrazol-1-yl)ethyl)-piperidine-1-carboxylate (27 mg). 25 mg of this material was stirred 0.75 h in MeOH/4M HCl in dioxane (1:1; 0.6 mL) and concentrated to give Int. 3E244 3-[3-methyl-2-oxo-5-[1-[2-(4-piperidyl)ethyl]pyrazol-4-yl]benzimidazol-1-yl]-piperidine-2,6-dione (26 mg, HCl salt).

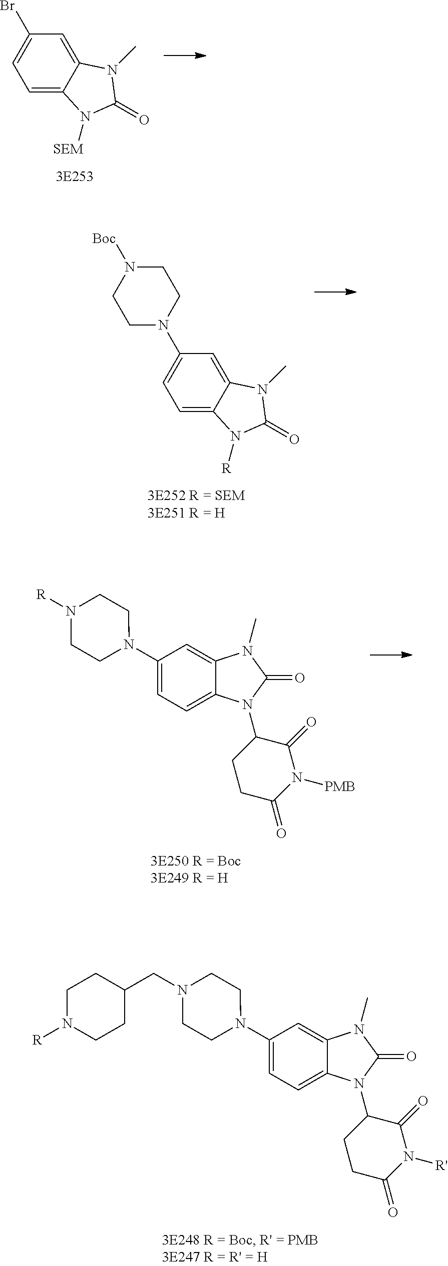

6-Bromo-1-methyl-1,3-dihydro-2H-benzo[d]imidazol-2-one (5.0 g) and NaH (1.32 g) were stirred 0.5 h in DMF (50 mL) at 0° C. SEM-C1 (4.0 g) was added and stirring continued ON at 0° C. to RT. The OL (water/EtOAc) was washed with brine, dried, concentrated, and purified by FC (hexane/EtOAc 3:2) to give Int. 3E253 5-bromo-3-methyl-1-((2-(trimethylsilyl)ethoxy)methyl)-1,3-dihydro-2H-benzo[d]imidazol-2-one (6.5 g). This material, tert-butyl piperazine-1-carboxylate (5.1 g), Pd₂dba₃ (1.7 g), RuPhos (0.85 g), and NaOtBu (3.5 g) were degassed in dioxane (65 mL) and stirred ON at 90° C. Filtration, concentration, and purification by FC (EtOAc/hexane 1:1) to give Int. 3E252 tert-butyl 4-(3-methyl-2-oxo-1-((2-(trimethylsilyl)ethoxy)-methyl)-2,3-dihydro-1H-benzo[d]-imidazol-5-yl)piperazine-1-carboxylate (8.0 g). 2.0 g of this material and TBAF (9.6 g) were stirred ON in THF (20 mL) at 80° C. The OL (water/EtOAc) was dried, concentrated, and purified by FC (hexane/EtOAc 3:7) to give Int. 3E251 tert-butyl 4-(3-methyl-2-oxo-2,3-dihydro-1H-benzo[d]-imidazol-5-yl)piperazine-1-carboxylate (1.1 g). This material and KOtBu (0.48 g) were stirred 0.5 h in THF (20 mL) at 0° C. Int. 3A8 (2.5 g) was added and stirring continued 2 h at 0° C. to RT. The OL (sat. aq. NH₄Cl/EtOAc) was dried, concentrated, and purified by FC (pentane/EtOAc 1:0 to 3:7) to give Int. 3E250 tert-butyl 4-[1-[1-[(4-methoxy-phenyl)methyl]-2,6-dioxo-3-piperidyl]-3-methyl-2-oxo-benzimidazol-5-yl]piperazine-1-carboxylate (0.9 g). Int. 3E250 (2.7 g) was stirred 2 h in 1.5M HCl in dioxane (40 mL) at 0° C. and concentrated to give Int. 3E249 1-[(4-methoxy-phenyl)-methyl]-3-(3-methyl-2-oxo-5-piperazin-1-yl-benzimidazol-1-yl)-piperidine-2,6-dione (1.8 g, HCl salt). 0.40 g of this material and tert-butyl 4-formyl-piperidine-1-carboxylate (0.22 g) were stirred 3 h in DMSO/DIPEA (13.3:1, 8.6 mL). STAB (0.55 g) was added at 0° C. and stirring continued 72 h at 0° C. to RT. The mixture was combined with another batch prepared from 1.0 g Int. 3E249. The OL (water/EtOAc) was washed with brine, dried, concentrated, and purified by FC (DCM/MeOH 95:5) to give on Int. 3E248 tert-butyl 4-[[4-[1-[1-[(4-methoxyphenyl)-methyl]-2,6-dioxo-3-piperidyl]-3-methyl-2-oxo-benzi-midazol-5-yl]-piperazin-1-yl]methyl]-piperidine-1-carboxylate (1.1 g). 0.8 g of this material was stirred 2 h in TFA/TfOH (2:1, 15 mL) at 0° C. to 80° C. and concentrated. The residue was triturated in Et₂O and HPLC-purified to give Int. 3E247 3-[3-methyl-2-oxo-5-[4-(4-piperidylmethyl)-piperazin-1-yl]benzimidazol-1-yl]-piperidine-2,6-dione (0.21 g, TfOH salt).

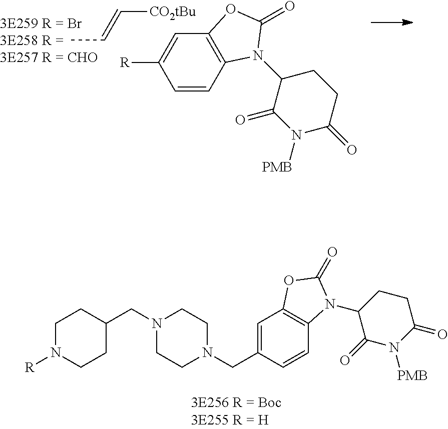

6-Bromo-3H-1,3-benzoxazol-2-one (2.0 g) and KOtBu (1.57 g) were stirred 0.5 h in THF (35 mL) at 0° C. Int. 3A8 (6.7 g) was added and stirring continued 3 h at 0° C. The OL (water/EtOAc) was washed with brine, dried, concentrated, and purified by FC (pentane/EtOAc 5:1 to 4:1) to give Int. 3E259 3-(6-bromo-2-oxo-1,3-benzoxazol-3-yl)-1-[(4-methoxyphenyl)methyl]piperidine-2,6-dione (4.1 g). 2.0 g of this material, tert-butyl acrylate (1.6 mL), Pd(OAc)₂ (0.15 g), and P(o-tol)₃ (0.27 g) were degassed in DMF/Et₃N (10:1, 21.9 mL) and stirred ON at 110° C. The OL (water/EtOAc) was washed with brine, dried, concentrated, and purified by FC (heptane/EtOAc 5:1 to 4:1) to give Int. 3E58 tert-butyl (E)-3-[3-[1-[(4-methoxyphenyl)methyl]-2,6-dioxo-3-piperidyl]-2-oxo-1,3-benzoxazol-6-yl]prop-2-enoate (1.5 g). This material, OsO₄ (0.27 g), and NaIO₄ (1.1 g) were stirred ON in THF/water (2:1; 18 mL). The OL (water/EtOAc) was washed with sat. aq. NaHCO₃ and brine, dried, concentrated, and purified by FC (pentane/EtOAc 1:1 to 2:3) to give Int. 3E257 3-[1-[(4-methoxyphenyl)methyl]-2,6-dioxo-3-piperidyl]-2-oxo-1,3-benzoxazole-6-carbaldehyde (0.6 g). Ints. 3E5/3E257 (0.41 g/0.30 g) were stirred 2 h in DCE/DIPEA (40:1, 15.4 mL) at 0° C. STAB (0.3 g) was added and stirring continued ON at 0° C. to RT. Int. 3E5 (0.41 g) and DIPEA (0.3 mL) were added and stirring continued at 2 h. STAB (0.30 g) was added and stirring continued ON. The OL (water/EtOAc) was washed with brine, dried, concentrated, and purified by FC (pentane/EtOAc 1:1 and 2:3) to give 3E256 tert-butyl 4-((4-((3-(1-(4-methoxy-benzyl)-2,6-dioxopiperidin-3-yl)-2-oxo-2,3-dihydro-benzo[d]oxazol-6-yl)methyl)-piperazin-1-yl)methyl)piperidine-1-carboxylate (0.6 g). A solution of this material in 1.5M HCl in dioxane (16 mL) was stirred 3 h at 0° C. to RT and concentrated to give Int. 3E255 1-(4-methoxybenzyl)-3-(2-oxo-6-((4-(piperidin-4-yl-methyl)-piperazin-1-yl)-methyl)benzo[d]oxazol-3 (2H)-yl)-piperidine-2,6-dione (0.48 g, HCl salt).

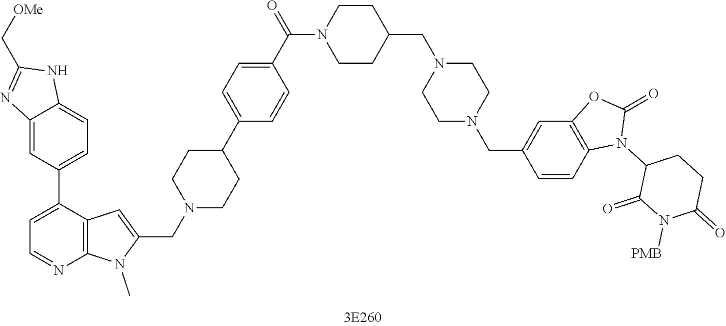

Ints. 3E255/3E261 (40 mg/42 mg) and HATU (31 mg) were stirred ON in DMF/DIPEA (1:0.06, 2.1 mL). The OL (water/EtOAc) was concentrated and purified by FC (DCM/MeOH 1:0 to 3:1) to give Int. 3E260 1-(4-methoxybenzyl)-3-(6-((4-((1-(4-(1-((4-(2-(methoxymethyl)-1H-benzo[d]imidazol-5-yl)-1-methyl-1H-pyrrolo[2,3-b]pyridin-2-yl)methyl)piperidin-4-yl)benzoyl)piperidin-4-yl)methyl)piperazin-1-yl)methyl)-2-oxobenzo[d]oxazol-3 (2H)-yl)piperidine-2,6-dione (48 mg).

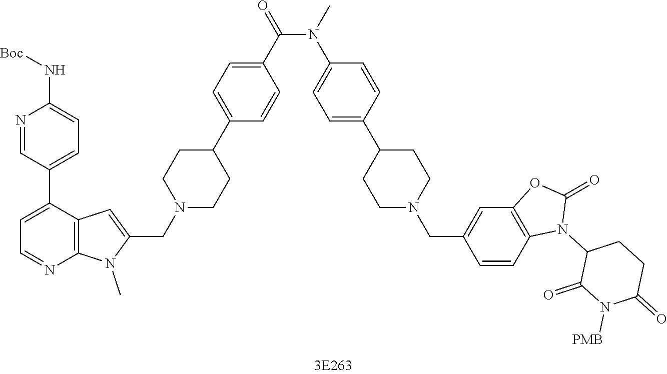

Int. 3E264 (20 mg), HATU (30 mg), and DIPEA (0.12 mL) were added and stirring continued ON. The OL (water/EtOAc) was concentrated and purified by FC (DCM/MeOH 1:0 to 9:1) to give Int. 3E263 tert-butyl (5-(2-((4-(4-((4-(1-((3-(1-(4-methoxybenzyl)-2,6-dioxopiperidin-3-yl)-2-oxo-2,3-dihydrobenzo[d]oxazol-6-yl)methyl)piperidin-4-yl)phenyl)(methyl)carbamoyl)phenyl)piperidin-1-yl)methyl)-1-methyl-1H-pyrrolo[2,3-b]pyridin-4-yl)pyridin-2-yl)carbamate (28 mg).

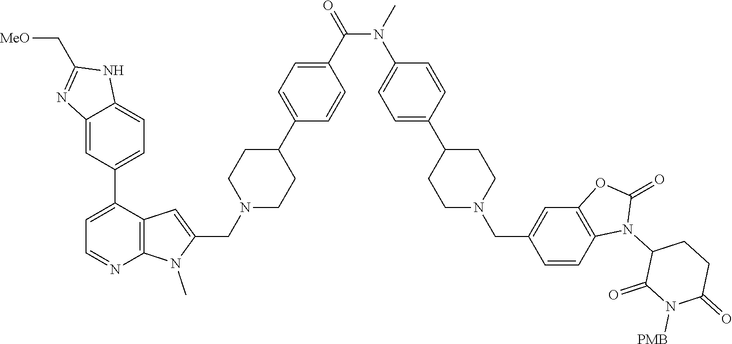

Ints. 3E264/3E261 (40 mg/41 mg) and HATU (31 mg) were stirred 72 h in DMF/DIPEA (16.7:1, 2.1 mL). Int. 3E264 (19 mg), HATU (25 mg), and DIPEA (0.12 mL) were added and stirring continued ON. The OL (water/EtOAc) was washed with 3M aq. CaCl₂ and brine, dried, concentrated, and purified by FC (DCM/MeOH 1:0 to 4:1) to give Int. 3E268 4-[1-[[4-[2-(methoxymethyl)-1H-benzimidazol-5-yl]-1-methyl-pyrrolo[2,3-b]pyridin-2-yl]methyl]-4-piperidyl]-N-[4-[1-[[3-[1-[(4-methoxyphenyl)methyl]-2,6-dioxo-3-piperidyl]-2-oxo-1,3-benzoxazol-6-yl]methyl]-4-piperidyl]phenyl]-N-methyl-benzamide (16 mg).

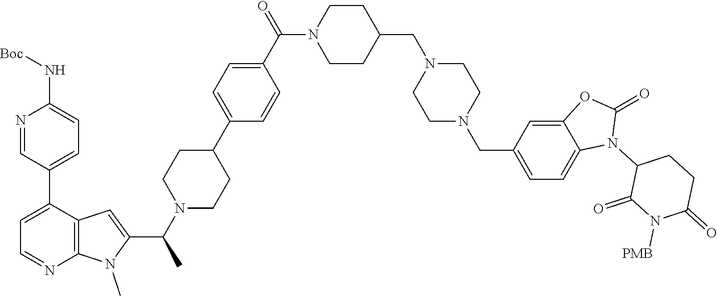

Ints. 3E76/3E255 (50 mg/51 mg) and HATU (39 mg) were stirred ON in DMF/DIPEA (12.51, 2.2 mL). The OL (water/EtOAc) was concentrated and purified by FC (DCM/MeOH/Et₃N 1:0:0 to 80:19.2:0.8) to give Int. 3E269 tert-butyl (5-(2-((1S)-1-(4-(4-(4-((4-((3-(1-(4-methoxybenzyl)-2,6-dioxopiperidin-3-yl)-2-oxo-2,3-dihydrobenzo[d]-oxazol-6-yl)methyl)piperazin-1-yl)methyl)piperidine-1-carbonyl)phenyl)piperidin-1-yl)ethyl)-1-methyl-1H-pyrrolo[2,3-b]pyridin-4-yl)pyridin-2-yl)carbamate (59 mg).

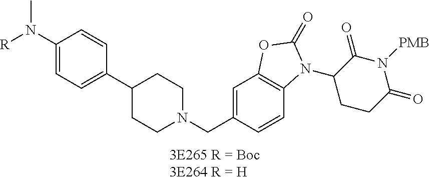

Ints. 3E257/3E266 (0.32 g/0.42 g) were stirred 2 h in DCE/DIPEA (25:1, 10.4 mL) at 0° C. to RT. STAB (0.33 g) was added and stirring continued ON at 0° C. to RT. The OL (water/EtOAc) was washed with brine, dried, concentrated, and purified by FC (pentane/EtOAc 1:1 to 2:3) to give Int. 3E265 tert-butyl N-[4-[1-[[3-[1-[(4-methoxyphenyl)methyl]-2,6-dioxo-3-piperidyl]-2-oxo-1,3-benzoxazol-6-yl]methyl]-4-piperidyl]-phenyl]-N-methyl-carbamate (0.43 g). A solution of 0.13 g of this material in 1.3M HCl in dioxane (4.5 mL) was stirred 3 h at 0° C. to RT and concentrated to give Int. 3E264 1-[(4-methoxy-phenyl)methyl]-3-[6-[[4-[4-(methyl-amino)phenyl]-1-piperidyl]-methyl]-2-oxo-1,3-benzoxazol-3-yl]-piperidine-2,6-dione (80 mg, HCl salt).

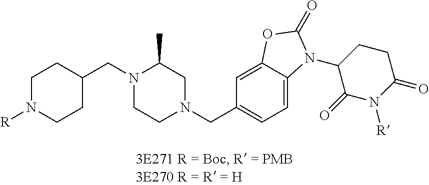

Ints. 3E74/3E257 (0.28 g/0.25 g) were stirred ON in DCE/DIPEA (24:1, 8.3 mL). STAB (0.40 g) was added and stirring continued ON. The OL (water/DCM) was dried, concentrated, and purified by FC (pentane/EtOAc 1:1) to give Int. 3E271 tert-butyl 4-[[(2S)-4-[[3-[1-[(4-methoxyphenyl)methyl]-2,6-dioxo-3-piperidyl]-2-oxo-1,3-benz-oxazol-6-yl]methyl]-2-methyl-piperazin-1-yl]methyl]piperidine-1-carboxylate (0.30 g). A solution of this material in TFA/TfOH (10:1; 3.3 mL) was stirred 1 h at 80° C. and concentrated to give Int. 3E270 3-[6-[[(3S)-3-methyl-4-(4-piperidylmethyl)piperazin-1-yl]methyl]-2-oxo-1,3-benzoxazol-3-yl]piperidine-2,6-dione (0.25 g, TfOH salt).

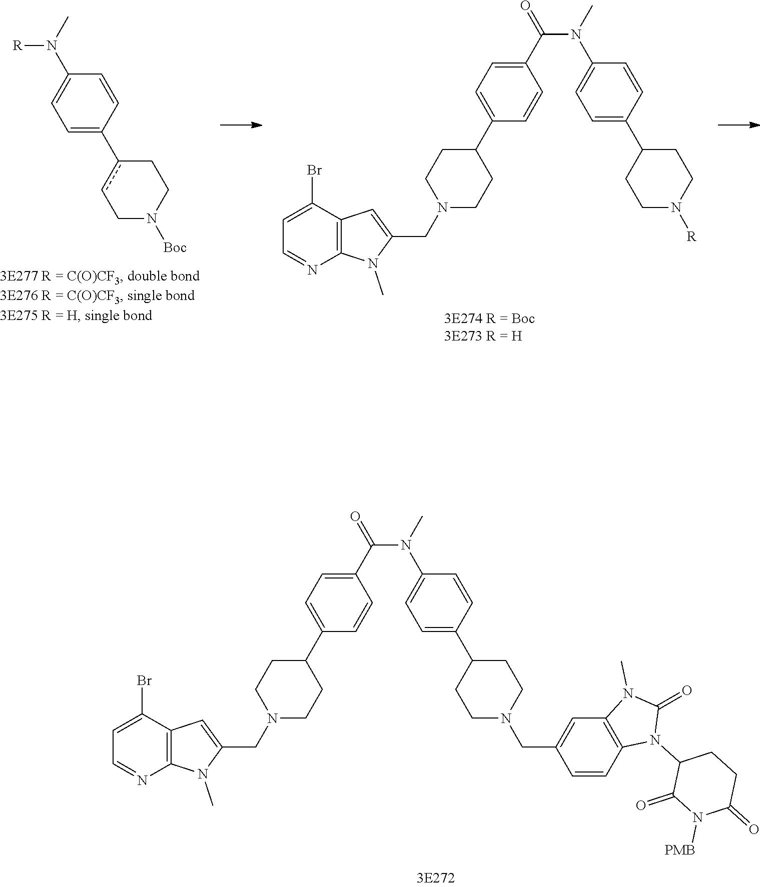

N-(4-bromophenyl)-2,2,2-trifluoro-N-methylacetamide (3.5 g), PdDPPFCl₂-DCM (0.44 g), tert-butyl 4-(4,4,5,5-tetramethyl-1,3,2-dioxaborolan-2-yl)-3,6-dihydropyridine-1 (2H)-carboxylate (3.3 g), and NaHCO₃ (1.8 g) were degassed in dioxane/water (10:1, 34 mL) and stirred ON at 100° C. and filtered. The OL (EtOAc/water) was washed with brine, dried, and concentrated. The residue was mixed with two additional batches prepared similarly and purified by FC (pentane/EtOAc 9:1 to 85:15) to give Int. 3E277 tert-butyl 4-(4-(2,2,2-trifluoro-N-methyl-acetamido)phenyl)-3,6-dihydropyridine-1 (2H)-carboxylate (8.3 g). 3.0 g of this material was hydrogenated ON using 10% Pd/C in MeOH (30 mL), filtered, concentrated, and purified by FC (pentane/EtOAc 9:1 to 85:15) to give Int. 3E276 tert-butyl 4-(4-(2,2,2-trifluoro-N-methyl-acetamido)phenyl)piperidine-1-carbo-xylate (2.5 g). 0.8 g of this material and K₂CO₃ (0.7 g) were stirred 3 h in MeOH (7 mL) and concentrated. The OL (water/DCM) was dried and concentrated to give Int. 3E275 tert-butyl 4-(4-(methylamino)phenyl)-piperidine-1-carboxylate (0.5 g). Ints. 1AC4/3E275 (0.5 g/0.58 g) and HATU (0.67 g) were stirred ON in DMF/DIPEA (24.5:1, 15.6 mL) and diluted with water to precipitate Int. 3E274 tert-butyl 4-(4-(4-(1-((4-bromo-1-methyl-1H-pyrrolo-[2,3-b]pyridin-2-yl)methyl)piperidin-4-yl)-N-methyl-benzamido)-phenyl)piperidine-1-carboxylate (0.6 g). 0.18 g of this material was stirred 0.5 h in DCM/TFA (2:1; 1.5 mL), concentrated, and SCX-purified to give Int. 3E273 4-[1-[(4-bromo-1-methyl-pyrrolo[2,3-b]pyridin-2-yl)methyl]-4-piperidyl]-N-methyl-N-[4-(4-piperidyl)phenyl]-benzamide (0.15 g). Ints. 3E3/3E273 (0.99 g/0.15 g) and 4 Å MS and STAB (0.27 g) were stirred 2 h in DCE (4 mL) at 70° C. The mixture was purified by FC (DCM/MeOH 95:5) to give Int. 3E272 4-(1-((4-bromo-1-methyl-1H-pyrrolo[2,3-b]pyridin-2-yl)methyl)piperidin-4-yl)-N-(4-(1-((1-(1-(4-methoxybenzyl)-2,6-dioxopiperidin-3-yl)-3-methyl-2-oxo-2,3-dihydro-1H-benzo[d]imidazol-5-yl)methyl)piperidin-4-yl)phenyl)-N-methylbenzamide (41 mg).

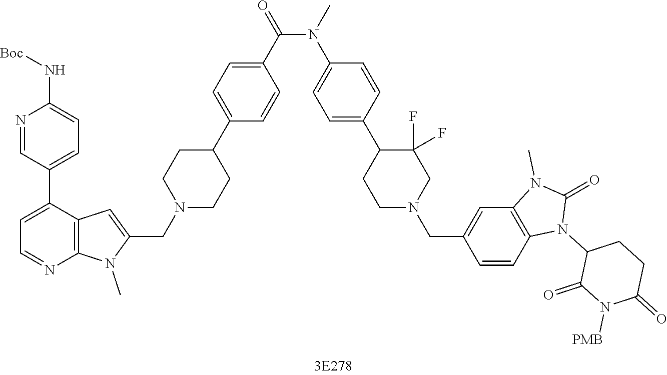

Int. 3E279 (49 mg), tert-butyl (5-(4,4,5,5-tetramethyl-1,3,2-dioxaborolan-2-yl)pyridin-2-yl)carbamate (21 mg), and PdDPPFCl₂-DCM (5 mg) were degassed in DMF/10% aq. Na₂CO₃ (10:1, 2.2 mL) and stirred 1.5 h at 90° C. The OL (water/EtOAc) was concentrated and purified by FC (DCM/MeOH 1:0 to 9:1) to give Int. 3E278 tert-butyl (5-(2-((4-(4-((4-(3,3-difluoro-1-((1-(1-(4-methoxybenzyl)-2,6-dioxopiperidin-3-yl)-3-methyl-2-oxo-2,3-dihydro-1H-benzo[d]imidazol-5-yl)methyl)piperidin-4-yl)phenyl)(methyl)-carbamoyl)phenyl)-piperidin-1-yl)methyl)-1-methyl-1H-pyrrolo[2,3-b]pyridin-4-yl)pyridin-2-yl)carbamate (37 mg).

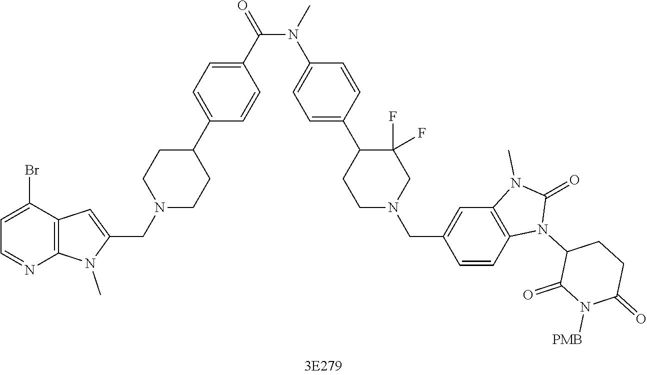

Ints. 3E3/3E280 (30 mg/52 mg) and 4 Å MS were stirred 0.5 h in DCE/DIPEA (100:1, 1.4 mL) at 70° C. STAB (78 mg) was added and stirring continued 3 h. The OL (water/DCM) was washed with brine, dried, concentrated, and purified by FC (DCM/MeOH 1:0 to 9:1) to give Int. 3E279 4-[1-[(4-bromo-1-methyl-pyrrolo-[2,3-b]pyridin-2-yl)methyl]-4-piperidyl]-N-[4-[3,3-difluoro-1-[[1-[1-[(4-methoxyphenyl)-methyl]-2,6-dioxo-3-piperidyl]-3-methyl-2-oxo-benzimidazol-5-yl]methyl]-4-piperidyl]-phenyl]-N-methyl-benzamide (49 mg).

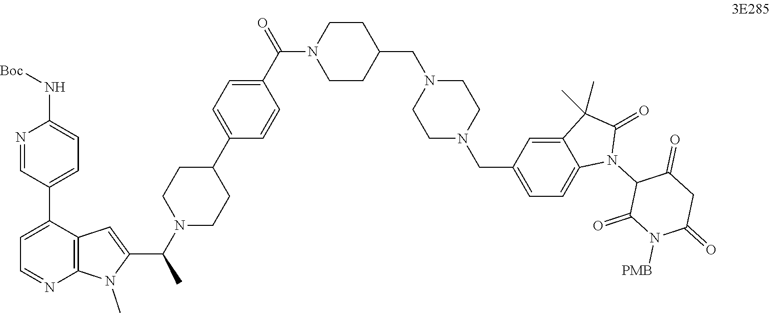

Int. 3E287 (50 mg) was stirred ON in 2.4M HCl in dioxane (5 mL) and concentrated. The residue, Int. 3E76 (55 mg), and HATU (49 mg) were stirred ON in DMF/DIPEA (15:1, 1.6 mL). The OL (EtOAc/water) was dried, concentrated, and purified by FC (DCM/MeOH 1:0 to 95:5) to give Int. 3E285 tert-butyl (5-(2-((1S)-1-(4-(4-(4-((4-((1-(1-(4-methoxybenzyl)-2,6-dioxopiperidin-3-yl)-3,3-dimethyl-2-oxoindolin-5-yl)methyl)piperazin-1-yl)methyl)piperidine-1-carbonyl)phenyl)piperidin-1-yl)ethyl)-1-methyl-1H-pyrrolo[2,3-b]pyridin-4-yl)pyridin-2-yl)carbamate (65 mg).

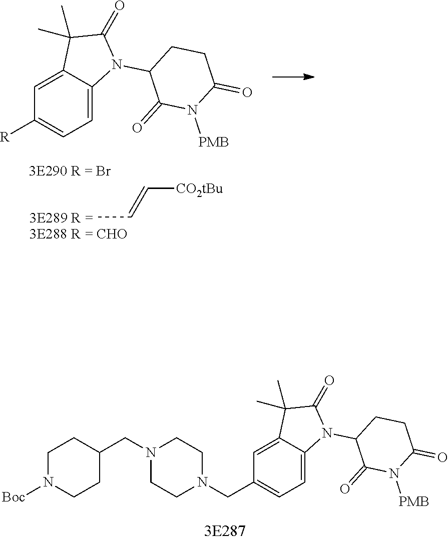

KOtBu (0.68 g) was added to a solution of 5-bromo-3,3-dimethyl-indolin-2-one (1.0 g) in THF (15 mL) at 0° C. Int. 3A8 (2.3 g) was added and stirring continued 2 h at 0° C. The OL (sat. aq. NH₄Cl/EtOAc) was dried, concentrated, and purified by FC (pentane/EtOAc 4:1) to give Int. 3E290 3-(5-bromo-3,3-dimethyl-2-oxo-indolin-1-yl)-1-[(4-methoxyphenyl)methyl]piperidine-2,6-dione (1.0 g). 0.4 g of this material, tert-butyl acrylate (0.33 g), Pd(OAc)₂ (19 mg), and P(o-tol)₃ (52 mg) were degassed in DMF/Et₃N (173:1, 50 mL) and stirred ON at 120° C. The OL (water/EtOAc) was washed with brine, dried, concentrated, and purified by FC (pentane/EtOAc 1:0 to 3:2) to give Int. 3E289 tert-butyl (E)-3-[1-[1-[(4-methoxyphenyl)methyl]-2,6-dioxo-3-piperidyl]-3,3-dimethyl-2-oxo-indolin-5-yl]prop-2-enoate (0.35 g). 0.3 g of this material, NaIO₄ (0.12 g), and OsO₄ (14 mg) were stirred ON in THF/water (2:1; 9 mL). The OL (EtOAc/sat. aq. Na₂S₂O₃) was concentrated and purified by FC (pentane/EtOAc 1:0 to 7:3) to give Int. 3E288 1-[1-[(4-methoxyphenyl)-methyl]-2,6-dioxo-3-piperidyl]-3,3-dimethyl-2-oxo-indoline-5-carbaldehyde (70 mg). Ints. 3E5/3E288 (40 mg/50 mg) were stirred 3 h in DCE/DIPEA (43:1, 3 mL). STAB (46 mg) was added and stirring continued ON at 0° C. to RT. The OL (water/DCM) was concentrated and purified by FC (DCM/MeOH 92:5 to 9:1) to give 3E287 tert-butyl 4-[[4-[[1-[1-[(4-methoxyphenyl)-methyl]-2,6-dioxo-3-piperidyl]-3,3-dimethyl-2-oxo-indolin-5-yl]methyl]-piperazin-1-yl]methyl]piperidine-1-carboxylate (40 mg).

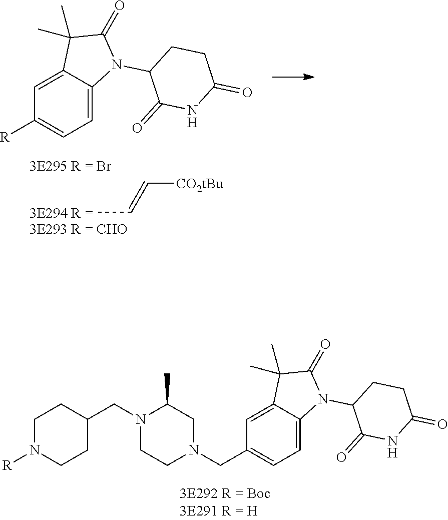

NaH (60% oil dispersion, 1.7 g) was added to a solution of 5-bromo-3,3-dimethyl-indolin-2-one (2.5 g) in THF (10 mL) at 0° C. 3-Bromopiperidine-2,6-dione (8.0 g) was added and stirring continued ON at 60° C. The OL (water/EtOAc) was dried, concentrated, and purified by FC (hexane/EtOAc 7:3 to 1:1) to give Int. 3E295 3-(5-bromo-3,3-dimethyl-2-oxo-indolin-1-yl)piperidine-2,6-dione (1.5 g). Int. 3E295 (1.8 g), tert-butyl acrylate (3.0 mL), Pd(OAc)₂ (92 mg), and P(o-tol)₃ (0.31 g) were degassed in DMF/Et₃N (4.8:1, 12 mL) and stirred ON at 120° C. The OL was (water/EtOAc) washed with brine, dried, and concentrated to give Int. 3E294 tert-butyl (E)-3-[1-(2,6-dioxo-3-piperidyl)-3,3-dimethyl-2-oxo-indolin-5-yl]prop-2-enoate (1.5 g). 0.5 g of this material, NaIO₄ (1.1 g), and OsO₄ (0.1 g) were stirred ON in THF/water (3:1; 20 mL). The OL (EtOAc/sat. aq. Na₂S₂O₃) was washed with sat. aq. NaHCO₃, dried, and concentrated to give Int. 3E293 1-(2,6-dioxo-3-piperidyl)-3,3-dimethyl-2-oxo-indoline-5-carbaldehyde (0.18 g). Ints. 3E74/3E293 (0.25 g/0.10 g) were stirred 5 h in THF/1M ZnCl₂ in THF (1:1, 2 mL). NaCNBH₃ (41 mg) was added and stirring continued ON. The OL (water/EtOAc) was dried, concentrated, and purified by FC (DCM/MeOH 9:1) to give 3E292 tert-butyl 4-[[(2S)-4-[[1-(2,6-dioxo-3-piperidyl)-3,3-dimethyl-2-oxo-indolin-5-yl]-methyl]-2-methyl-piperazin-1-yl]methyl]piperidine-1-carboxylate (0.11 g). Int. 3E292 (0.12 g) was stirred 3 h in DCM/4M HCl in dioxane (6:1; 6.2 mL) at 0° C. to RT and concentrated to give Int. 3E291 3-(3,3-dimethyl-5-(((S)-3-methyl-4-(piperidin-4-yl-methyl)piperazin-1-yl)methyl)-2-oxoindolin-1-yl)piperidine-2,6-dione (0.11 g, HCl salt).

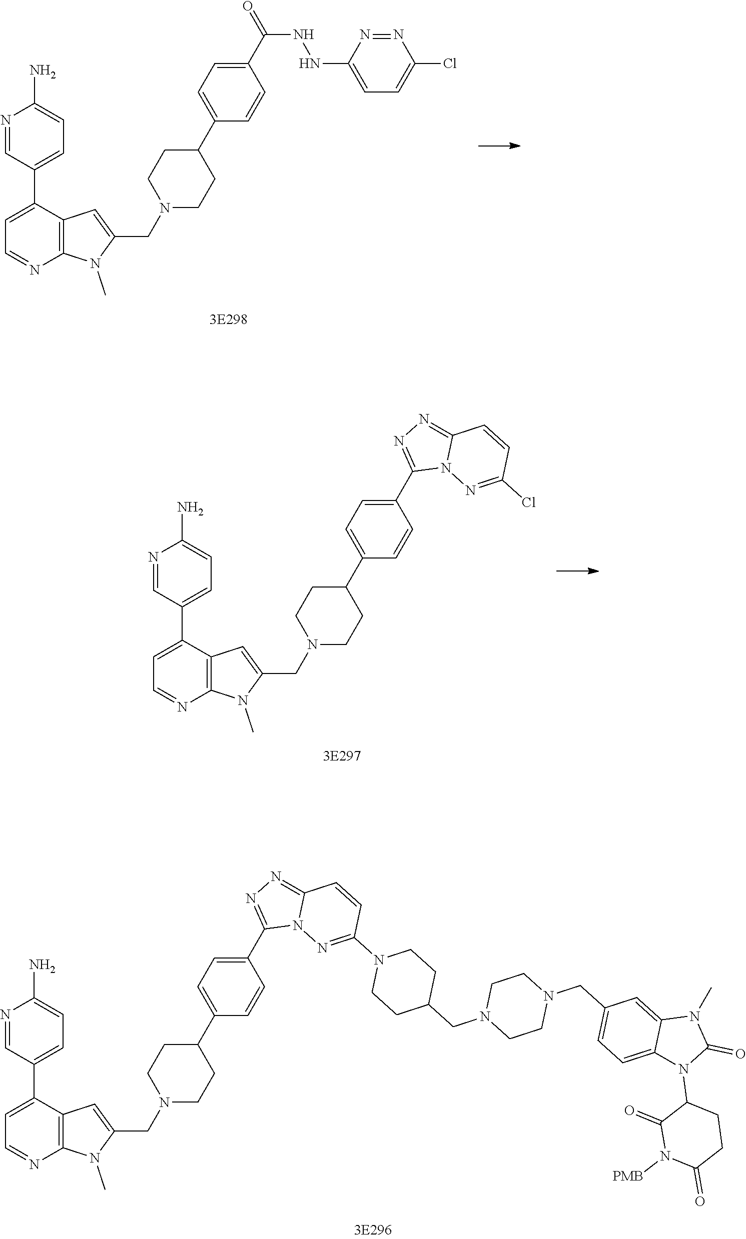

Int. 3E2 (80 mg) was stirred 0.3 h in DCM/TFA (4:1, 2.5 mL) and concentrated to give Int. 3E299 3-[3-methyl-2-oxo-4-[[4-(4-piperidylmethyl)piperazin-1-yl]methyl]-benzimidazol-1-yl]piperidine-2,6-dione (85 mg, TFA salt). Int. 3E23 (0.17 g) and HATU (0.2 g) were stirred 4 h in DMF/DIPEA (5.6:1, 1.8 mL). 3-Chloro-6-hydrazineyl-pyridazine (67 mg) was added and stirring continued ON. The OL (2MeTHF/water) was washed with brine, dried, concentrated, and purified by FC (DCM/MeOH/Et₃N 1:0:0 to 90:8.5:0.5) to give Int. 3E298 4-[1-[[4-(6-amino-3-pyridyl)-1-methyl-pyrrolo[2,3-b]-pyridin-2-yl]methyl]-4-piperidyl]-N′-(6-chloropyridazin-3-yl)benzohydrazide (0.12 g). This material was stirred 3 h in AcOH (4 mL) at 90° C., concentrated, and purified by FC (DCM/MeOH/Et₃N 95:4.5:0.5) to give Int. 3E297 5-[2-[[4-[4-(6-chloro-[1,2,4]triazolo[4,3-b]pyridazin-3-yl)phenyl]-1-piperidyl]-methyl]-1-methyl-pyrrolo[2,3-b]pyridin-4-yl]pyridin-2-amine (77 mg). Ints. 3E299/3E297 (57 mg/38 mg) were stirred 1.5 h in NMP/DIPEA (42:1, 2.05 mL) at 90° C., 48 h at 100° C., and 4 h at 120° C. The OL (2MeTHF/water) was washed with brine, dried, concentrated, and purified by FC (DCM/MeOH/Et₃N 95:4.5:0.5) to give Int. 3E296 (40 mg).

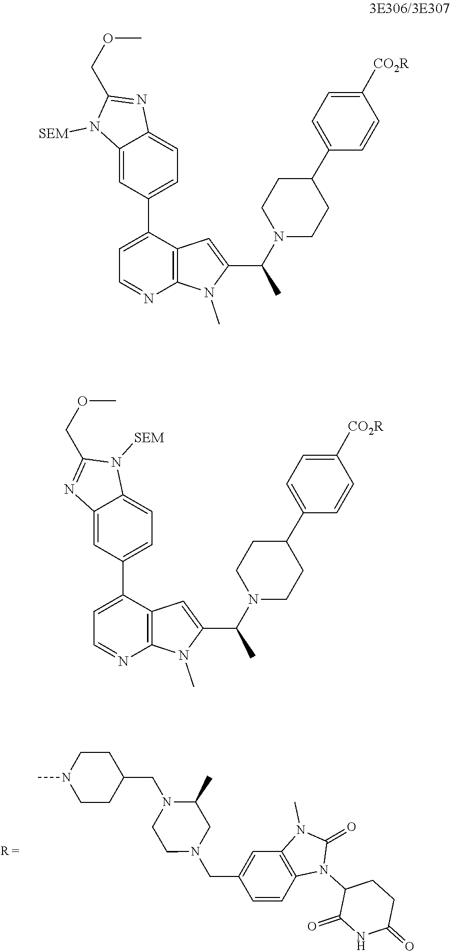

Ints. 3E68/3E308/3E309 (0.11 g/0.12 g) and HATU (91 mg) were stirred ON in DMF/DIPEA (18.4:1. 3.7 mL) and diluted with water to precipitate Int. 3E306/3E307 3-(5-(((S)-4-((1-(4-(1-((S)-1-(4-(2-(methoxymethyl)-1-((2-(trimethylsilyl)ethoxy)methyl)-1H-benzo[d]imidazol-6-yl)-1-methyl-1H-pyrrolo[2,3-b]pyridin-2-yl)ethyl)piperidin-4-yl)benzoyl)piperidin-4-yl)-methyl)-3-methylpiperazin-1-yl)methyl)-3-methyl-2-oxo-2,3-dihydro-1H-benzo[d]imidazol-1-yl)piperidine-2,6-dione and 3-(5-(((S)-4-((1-(4-(1-((S)-1-(4-(2-(methoxymethyl)-1-((2-(trimethylsilyl)ethoxy)methyl)-1H-benzo[d]-imidazol-5-yl)-1-methyl-1H-pyrrolo[2,3-b]pyridin-2-yl)ethyl)piperidin-4-yl)benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione (0.12 g).

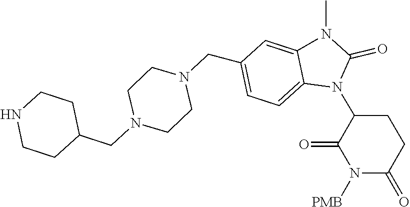

Int. 3E2 (80 mg) was stirred 0.3 h in DCM/TFA (4:1, 2.5 mL) and concentrated to give Int. 3E299 3-[3-methyl-2-oxo-4-[[4-(4-piperidylmethyl)piperazin-1-yl]methyl]-benzimidazol-1-yl]piperidine-2,6-dione (85 mg, TFA salt).

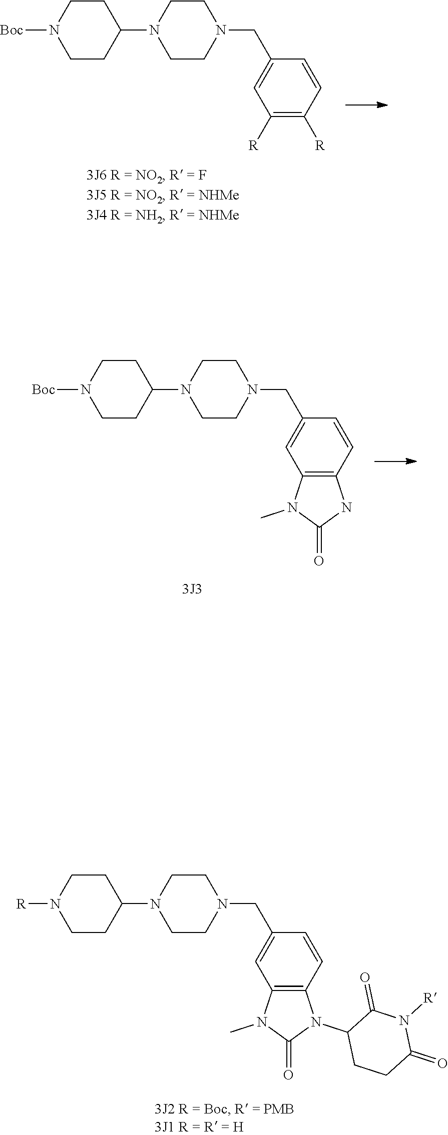

tert-Butyl 4-(piperazin-1-yl)piperidine-1-carboxylate (5.1 g) and 3-fluoro-4-nitro-benzaldehyde (3.0 g) were stirred 6 h in DCE/DIPEA (4.2:1, 62 mL). STAB (11 g) was added and stirring continued ON at 0° C. to RT. The OL (water/EtOAc) was washed with brine, dried, concentrated, and purified by FC (DCM/MeOH 95:5) to give Int. 3J6 tert-butyl 4-(4-(3-fluoro-4-nitrobenzyl)piperazin-1-yl)piperidine-1-carboxylate (4.0 g). This material was stirred in THF/2M MeNH₂ in MeOH (4.2:1, 50 mL) ON at 0° C. to RT, diluted with sat. aq. NaHCO₃, and partially concentrated. The product was extracted into EtOAc, dried, concentrated, and triturated in pentane to give Int. 3J5 tert-butyl 4-(4-(3-(methylamino)-4-nitrobenzyl)-piperazin-1-yl)-piperidine-1-carboxylate (4.0 g). This material was hydrogenated ON using Raney-nickel (1.3 g) in EtOH (50 mL), filtered, and concentrated to give Int. 3J4 tert-butyl 4-(4-(4-amino-3-(methylamino)benzyl)-piperazin-1-yl)piperidine-1-carboxylate (3.2 g). This material and CDI (2.6 g) were stirred in ACN (40 mL) at 80° C. and concentrated. The OL (water/EtOAc) was washed with brine, dried, concentrated, and purified by FC (DCM/MeOH 95:5) to give Int. 3J3 tert-butyl 4-(4-((3-methyl-2-oxo-2,3-dihydro-1λ²-benzo[d]imidazol-5-yl)-methyl)-piperazin-1-yl)piperidine-1-carboxylate (1.4 g). This material and KOtBu (0.47 g) were stirred 0.5 h in THF (20 mL). Int. 3A8 (3.2 g) was added and stirring continued 3 h at 0° C. The OL (water/EtOAc) was washed with brine, dried, concentrated, and purified by FC (DCM/MeOH 98:2 to 97:3) to give Int. 3J2 tert-butyl 4-(4-((1-(1-(4-methoxybenzyl)-2,6-dioxopiperidin-3-yl)-3-methyl-2-oxo-2,3-dihydro-1H-benzo[d]-imidazol-5-yl)methyl)-piperazin-1-yl)piperidine-1-carboxylate (1.2 g). 0.5 g of this material was stirred 1 h in TFA/TfOH (1:10, 5.5 mL) at 80° C., concentrated, and triturated in Et₂O to give Int. 3J1 3-(3-methyl-2-oxo-5-((4-(piperidin-4-yl)piperazin-1-yl)methyl)-2,3-dihydro-1H-benzo[d]imidazol-1-yl)piperidine-2,6-dione. This material was combined with another batch prepared similarly and HPLC-purified to give Int. 3J1 (0.24 g).

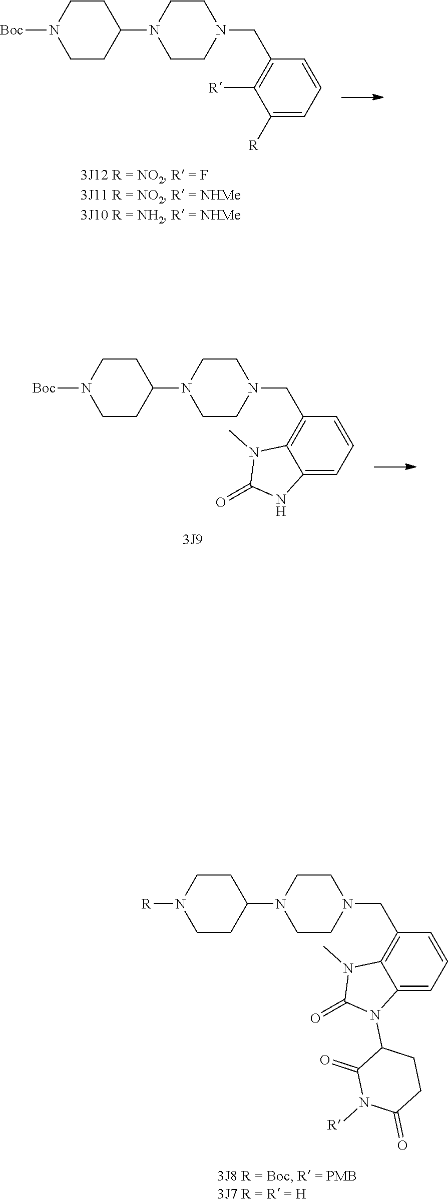

2-Fluoro-3-nitro-benzaldehyde (4.4 g) and tert-Butyl 4-(piperazin-1-yl)piperidine-1-carboxylate (8.4 g) were stirred 4 h in DCE/DIPEA (5.7:1, 97 mL). STAB (16 g) was added and stirring continued ON at 0° C. to RT and concentrated. The OL (sat. aq. NaHCO₃/DCM) was washed with brine, dried, concentrated, and purified by FC (DCM/MeOH 1:0 to 9:1) to give Int. 3J12 tert-butyl 4-(4-(2-fluoro-3-nitrobenzyl)-piperazin-1-yl)-piperidine-1-carboxylate (2.4 g). This material was stirred in THF/2M MeNH₂ in MeOH (4:1, 29 mL) ON and concentrated to give Int. 3J11 tert-butyl 4-[4-[[2-(methylamino)-3-nitro-phenyl]methyl]-piperazin-1-yl]piperidine-1-carboxylate (2.3 g). Int. 3J11 (0.30 g) was hydrogenated 2 h using 10% Pd(OH)₂/C (0.15 g) in MeOH (6 mL), filtered, and concentrated to give Int. 3J10 tert-butyl 4-[4-[[3-amino-2-(methylamino)-phenyl]-methyl]-piperazin-1-yl]-piperidine-1-carboxylate (0.25 g). Int 3J10 (2.1 g) and CDI (2.5 g) were stirred ON in ACN (25 mL) at 80° C. and concentrated. The OL (water/DCM/MeOH (9:1)) was washed with water and brine, dried, concentrated, and purified by FC (DCM/MeOH 1:0 to 9:1) to give Int. 3J9 tert-butyl 4-[4-[(3-methyl-2-oxo-1H-benzimidazol-4-yl)methyl]piperazin-1-yl]piperidine-1-carboxylate (1.6 g). Int. 3J9 (1.47 g) and KOtBu (0.42 g) were stirred 0.5 h in THF (40 mL) at −10° C. to 0° C. Int. 3A8 (3.2 g in THF (20 mL)) was added and stirring continued 3 h at −10° C. to 0° C. The OL (sat. aq. NH₄Cl/EtOAc) was washed with brine, dried, concentrated, and purified by FC (DCM/MeOH 1:0 to 9:1) to give Int. 3J8 tert-butyl 4-[4-[[1-[1-[(4-methoxy-phenyl)-methyl]-2,6-dioxo-3-piperidyl]-3-methyl-2-oxo-benzimidazol-4-yl]methyl]-piperazin-1-yl]piperidine-1-carboxylate (1.5 g). 0.3 g of this material was stirred 1 h in TFA/TfOH (10:1, 3.3 mL) at 80° C., concentrated, and triturated in Et₂O to give Int. 3J1. This material was combined with another batch prepared similarly from Int. 3J8 (0.60 g) and HPLC-purified to give Int.3J7 3-(3-methyl-2-oxo-4-((4-(piperidin-4-yl)piperazin-1-yl)methyl)-2,3-dihydro-1H-benzo[d]imidazol-1-yl)piperidine-2,6-dione (0.20 g).

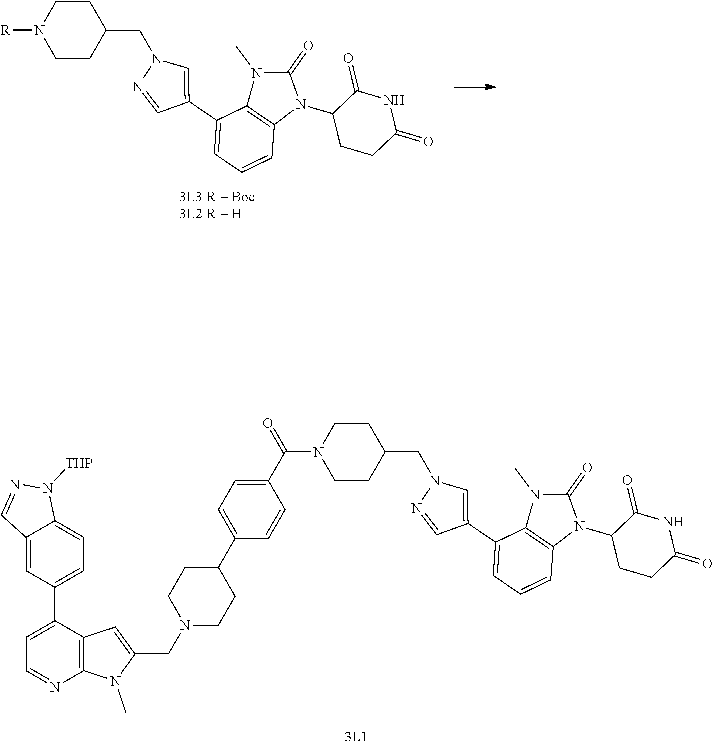

Int. 3A6 (40 mg), PdDPPFCl₂-DCM (10 mg), and tert-butyl 4-((4-(4,4,5,5-tetramethyl-1,3,2-dioxaborolan-2-yl)-1H-pyrazol-1-yl)methyl)piperidine-1-carboxylate (60 mg) were degassed in DMF/10% aq. Na₂CO₃ (5.6:1, 1.65 mL) and stirred 0.5 h at 100° C. The OL (water/EtOAc) was washed with brine, dried, concentrated, and purified by FC (EtOAc) to give Int. 3L3 tert-butyl 4-((4-(1-(2,6-dioxopiperidin-3-yl)-3-methyl-2-oxo-2,3-dihydro-1H-benzo[d]imidazol-4-yl)-1H-pyrazol-1-yl)methyl)piperidine-1-carboxylate (32 mg). Int. 3L3 (22 mg) was stirred 1 h in 2M HCl in dioxane (0.4 mL). The mixture was diluted with MeOH, concentrated, and co-concentrated with DCM to give Int. 3L2 3-(3-methyl-2-oxo-4-(1-(piperidin-4-ylmethyl)-1H-pyrazol-4-yl)-2,3-dihydro-1H-benzo[d]imidazol-1-yl)piperidine-2,6-dione. This material, Int. 3A2 (24 mg), and HATU (17 mg) were stirred 1 h in DMF/DIPEA (5.4:1, 0.41 mL). The mixture was HPLC-purified to give Int. 3L1 3-(3-methyl-4-(1-((1-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)-piperidin-4-yl)methyl)-1H-pyrazol-4-yl)-2-oxo-2,3-dihydro-1H-benzo[d]imidazol-1-yl)-piperidine-2,6-dione (27 mg).

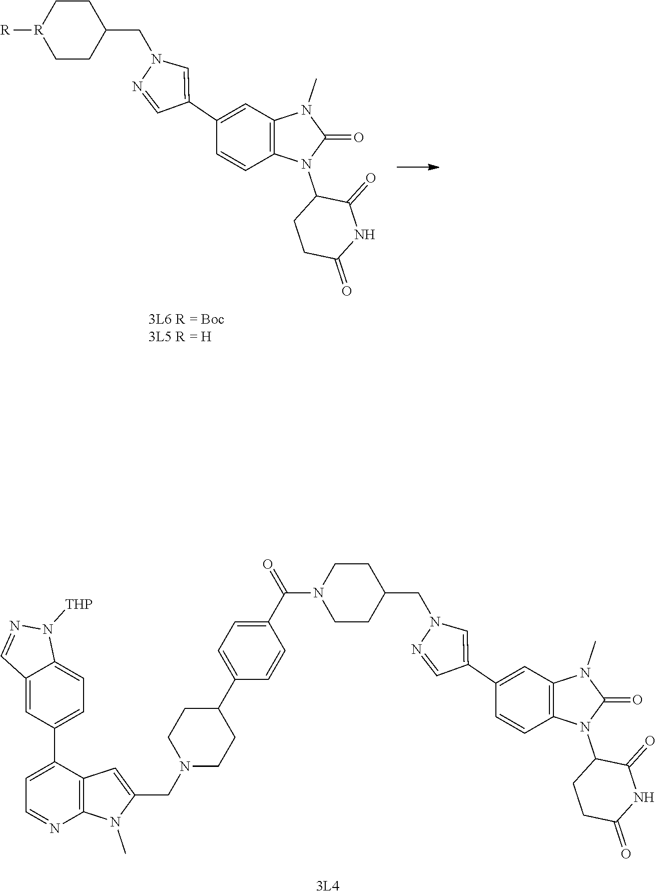

Int. 3B3 (40 mg), PdDPPFCl₂-DCM (10 mg), and tert-butyl 4-((4-(4,4,5,5-tetramethyl-1,3,2-dioxaborolan-2-yl)-1H-pyrazol-1-yl)methyl)piperidine-1-carboxylate (60 mg) were degassed in DMF/10% aq. Na₂CO₃ (5.6:1, 1.65 mL) and stirred 0.5 h at 100° C. The OL (water/EtOAc) was washed with brine, dried, concentrated, and purified by FC (EtOAc) to give Int. 3L6 tert-butyl 4-((4-(1-(2,6-dioxopiperidin-3-yl)-3-methyl-2-oxo-2,3-dihydro-1H-benzo[d]imidazol-5-yl)-1H-pyrazol-1-yl)methyl)piperidine-1-carboxylate (30 mg). Int. 3L6 (20 mg) was stirred 1 h in 2M HCl in dioxane (0.4 mL). The mixture was diluted with MeOH, concentrated, and co-concentrated with DCM to give Int. 3L5 3-(3-methyl-2-oxo-5-(1-(piperidin-4-ylmethyl)-1H-pyrazol-4-yl)-2,3-dihydro-1H-benzo[d]imidazol-1-yl)piperidine-2,6-dione. This material, Int. 3A2 (25 mg), and HATU (18 mg) were stirred 1 h in DMF/DIPEA (5.4:1, 0.41 mL). The mixture was HPLC-purified to give Int. 3L4 3-(3-methyl-5-(1-((1-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)piperidin-4-yl)methyl)-1H-pyrazol-4-yl)-2-oxo-2,3-dihydro-1H-benzo[d]imidazol-1-yl)piperidine-2,6-dione (24 mg).

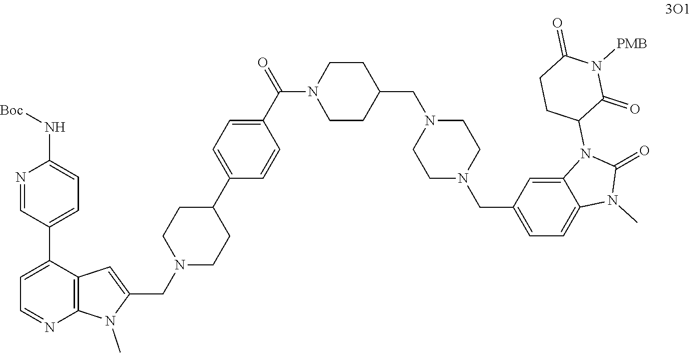

Int. 3O2 (0.99 g), tert-butyl N-[5-(4,4,5,5-tetramethyl-1,3,2-dioxaborolan-2-yl)-2-pyridyl]carbamate (48 mg), and PdDPPFCl₂-DCM (8 mg) were degassed in DMF/10% aq. Na₂CO₃ (5:1, 2.4 mL) and stirred 1.5 h at 90° C. The OL (EtOAc/water) was washed with brine, dried, concentrated, and purified by FC (DCM/MeOH 1:0 to 4:1) to give Int. 3O1 tert-butyl (5-(2-((4-(4-(4-((4-((3-(1-(4-methoxybenzyl)-2,6-dioxopiperidin-3-yl)-1-methyl-2-oxo-2,3-dihydro-1H-benzo[d]imidazol-5-yl)methyl)piperazin-1-yl)methyl)piperidine-1-carbonyl)phenyl)piperidin-1-yl)methyl)-1-methyl-1H-pyrrolo[2,3-b]pyridin-4-yl)pyridin-2-yl)carbamate (0.11 g).

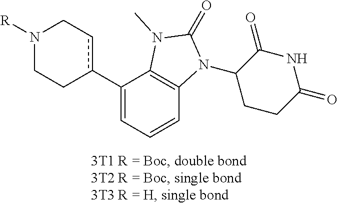

Int. 3A6 (0.10 g), tert-butyl 4-(4,4,5,5-tetramethyl-1,3,2-dioxaborolan-2-yl)-3,6-dihydropyridine-1 (2H)-carboxylate (0.12 g), and PdDPPFCl₂-DCM (24 mg) were degassed in DMF/10% aq. Na₂CO₃ (4.8:1, 3.6 mL) and stirred 0.5 h at 100° C. The OL (EtOAc/3M aq. CaCl₂) was washed with brine, dried, concentrated, and purified by FC (heptane to EtOAc) to give Int. 3T1 tert-butyl 4-[1-(2,6-dioxo-3-piperidyl)-3-methyl-2-oxo-benzimidazol-4-yl]-3,6-dihydro-2H-pyridine-1-carboxylate (70 mg). 65 mg of this material was hydrogenated ON using 10% Pd/C (16 mg) and Pd(OH)₂ (10 mg) in THF/DMF (2:1, 3 mL), filtered, and purified by FC (heptane to EtOAc) to give Int. 3T2 tert-butyl 4-[1-(2,6-dioxo-3-piperidyl)-3-methyl-2-oxo-benzimidazol-4-yl]piperidine-1-carboxylate (22 mg). Int. 3T3 3-(3-methyl-2-oxo-4-(piperidin-4-yl)-2,3-dihydro-1H-benzo[d]imidazol-1-yl)piperidine-2,6-dione was obtained from Int. 3T1 by treatment with HCl.

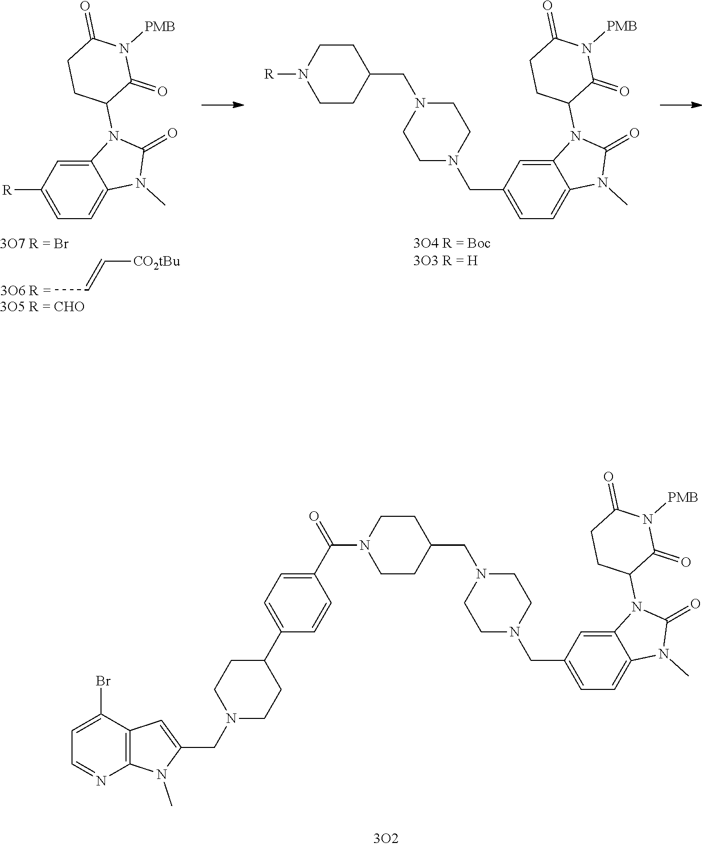

KOtBu (1.3 g) was added to a solution of 6-bromo-3-methyl-1H-benzimidazol-2-one (2.0 g) in THF (10 mL) at 0° C. and the mixture was stirred at RT for 0.5 h. Int. 3A8 (5.42 g) was added and stirring continued 3 h. The OL (sat. aq. NH₄Cl/EtOAc) was dried, concentrated, and purified by FC (hexane/EtOAc 1:0 to 2:3) and by trituration in Et₂O/EtOAc to give Int. 3O7 3-(6-bromo-3-methyl-2-oxo-benzimidazol-1-yl)-1-[(4-methoxyphenyl)methyl]-piperidine-2,6-dione (2.24 g). Int. 3O7 (0.5 g), tert-butyl acrylate (0.35 g), P(o-tol)₃ (66 mg), and Pd(OAc)₂ (25 mg) were degassed in DMF/Et₃N (10:1, 5.5 mL) and stirred 120° C. ON. The OL (water/EtOAc) was washed with brine, dried, concentrated, and purified by FC (heptane to EtOAc) to give Int. 3O6 tert-butyl (E)-3-(3-(1-(4-methoxybenzyl)-2,6-dioxopiperidin-3-yl)-1-methyl-2-oxo-2,3-dihydro-1H-benzo[d]imidazol-5-yl)acrylate (0.52 g). Int. 3O6 (0.5 g), NaIO₄ (0.57 g), and OsO₄ (0.11 g) were stirred ON in THF/water (2:1; 12 mL). The OL (sat. aq. NaHCO₃/EtOAc) was washed with brine, dried, concentrated, and purified by FC (heptane to EtOAc) to give Int. 3O5 3-(1-(4-methoxybenzyl)-2,6-dioxopiperidin-3-yl)-1-methyl-2-oxo-2,3-dihydro-1H-benzo[d]imidazole-5-carbaldehyde (0.31 g). Ints. 3O5/3E5 (0.30 g/0.31 g) were stirred 0.5 h in DCE/DIPEA (78:1, 10.1 mL) at 70° C. STAB (0.78 g) was added at RT and stirring continued 3 h at 70° C. The OL (water/DCM) was washed with brine, concentrated, and purified by FC (DCM/MeOH 1:0 to 9:1) to give Int. 3O4 tert-butyl 4-((4-((3-(1-(4-methoxy-benzyl)-2,6-dioxopiperidin-3-yl)-1-methyl-2-oxo-2,3-dihydro-1H-benzo[d]imidazol-5-yl)methyl)piperazin-1-yl)-methyl)piperidine-1-carboxylate (0.43 g). Int. 3O4 (0.17 g) was stirred 0.5 h in DCM/TFA (2:1, 3 mL) and concentrated to give Int. 3O3 1-[(4-methoxyphenyl)methyl]-3-[3-methyl-2-oxo-6-[[4-(4-piperidylmethyl)-piperazin-1-yl]methyl]benzimidazol-1-yl]piperidine-2,6-dione (0.14 g, TFA salt). Ints. 1AC4/3O3 (71 mg/79 mg) and HATU (68 mg) were stirred ON in DMF/DIPEA (8.3:1, 2.2 mL). The OL (EtOAc/water) was washed with brine, concentrated, and purified by FC (DCM/MeOH 1:0 to 85:15) to give Int. 3O2 3-[6-[[4-[[1-[4-[1-[(4-bromo-1-methyl-pyrrolo[2,3-b]pyridin-2-yl)methyl]-4-piperidyl]benzoyl]-4-piperidyl]methyl]piperazin-1-yl]methyl]-3-methyl-2-oxo-benzimidazol-1-yl]-1-[(4-methoxyphenyl)methyl]piperidine-2,6-dione (0.99 g).

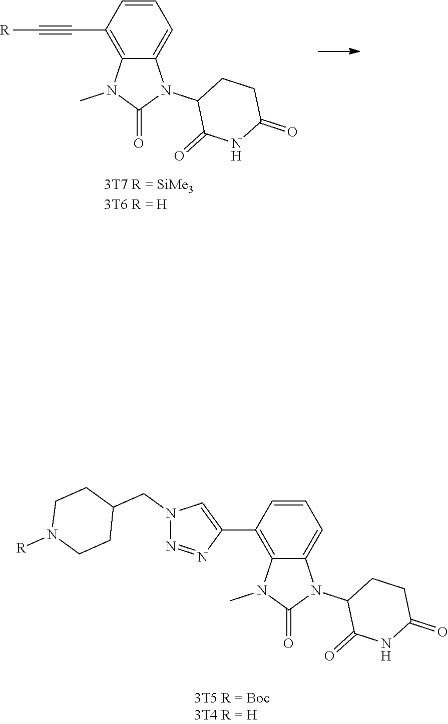

Int. 3A6 (0.25 mg), ethynyltrimethylsilane (0.18 g), CuI (0.15 g), and PdDPPFCl₂-DCM (0.11 g) were degassed in DMF/Et₃N (5:1, 4.8 mL) and stirred 5 h at 70° C. The OL (water/EtOAc) was washed with brine, dried, and concentrated. The residue was purified by FC (heptane/EtOAc 1:0 to 1:1) and by HPLC to give Int. 3T7 3-(3-methyl-2-oxo-4-((trimethylsilyl)ethynyl)-2,3-dihydro-1H-benzo[d]imidazol-1-yl)piperidine-2,6-dione (28 mg). Int. 3T7 (43 mg) was stirred 0.5 h in 0.7M TBAF in THF (3.2 mL). The mixture was HPLC-purified to give Int. 3T6 3-(4-ethynyl-3-methyl-2-oxo-2,3-dihydro-1H-benzo[d]-imidazol-1-yl)piperidine-2,6-dione (26 mg). NaN₃ (0.11 g) and tert-butyl 4-(bromo-methyl)piperidine-1-carboxylate (0.40 g) were stirred ON in DMF (5 mL). The OL (water/EtOAc) was washed with brine, dried, and concentrated to give Int. 3T8 tert-butyl 4-(azidomethyl)piperidine-1-carboxylate (0.32 g). Ints. 3T8/3T6 (21 mg/22 mg), CuSO₄ (2 mg), ascorbic acid (9 mg) were degassed in tert-butanol/water (4:1, 1.5 mL) and stirred 2 h at 80-90° C. The mixture was diluted with DMSO/MeOH (2:1, 3 mL) and HPLC-purified to give Int. 3T5 tert-butyl 4-((4-(1-(2,6-dioxopiperidin-3-yl)-3-methyl-2-oxo-2,3-dihydro-1H-benzo[d]imidazol-4-yl)-1H-1,2,3-triazol-1-yl)methyl)piperidine-1-carboxylate (27 mg). Int. 3T5 (23 mg) was stirred 1 h on DCM/TFA (2:1, 3 mL). The mixture was concentrated to give Int. 3T4 3-(3-methyl-2-oxo-4-(1-(piperidin-4-yl-methyl)-1H-1,2,3-triazol-4-yl)-2,3-dihydro-1H-benzo[d]imidazol-1-yl)piperidine-2,6-dione (18 mg, TFA salt).

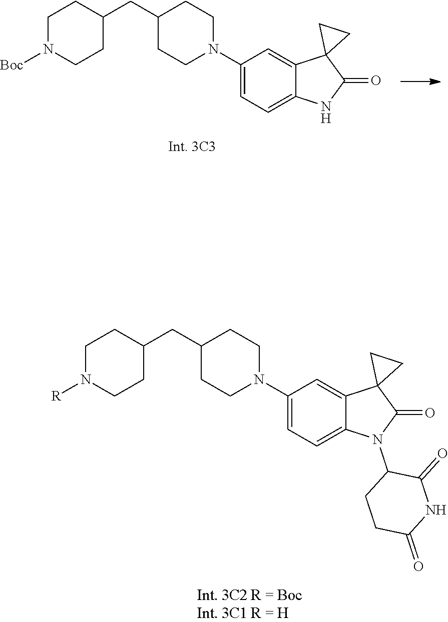

tert-Butyl 4-(piperidin-4-ylmethyl)piperidine-1-carboxylate (1.3 g), 5′-bromospiro[cyclopropane-1,3′-indolin]-2′-one (1 g) and RuPhos (0.27 g) were mixed in toluene (20 mL) under an inert atmosphere. 1.0 M LiHMDs in THF (17 mL) and RuPhos Pd-G4 (0.36 g) were added and the mixture was stirred 4 h at 90° C. The OL (aq. NH₄Cl/EtOAc) was dried, concentrated, and purified by FC (PE/EtOAc 2:3 to 1:4) to give Int. 3C3 tert-butyl 4-((1-(2′-oxospiro[cyclopropane-1,3′-indolin]-5′-yl)piperidin-4-yl)methyl)piperidine-1-carboxylate (1.4 g). 3-Bromopiperidine-2,6-dione (2.2 g) was added to a solution of Int. 3C3 (1 g) and 1.0 M LiHMDs in THF (11 mL) in THF (15 mL) and stirred 1 h at 55° C. The OL (water/EtOAc) was dried, concentrated, and purified by FC (PE/EtOAc 1:4) to give Int. 3C2 tert-butyl 4-((1-(1′-(2,6-dioxopiperidin-3-yl)-2′-oxospiro[cyclopropane-1,3′-indolin]-5′-yl)-piperidin-4-yl)methyl)piperidine-1-carboxylate (0.8 g). Int. 3C2 (0.90 g) was stirred 16 h in DCM/TFA (10:1, 22 mL) at 0° C. to RT, concentrated, and triturated in Et₂O to give Int. 3C1 3-(2′-oxo-5′-(4-(piperidin-4-ylmethyl)piperidin-1-yl)spiro[cyclopropane-1,3′-indolin]-1′-yl)piperidine-2,6-dione (0.90 g, TFA salt).

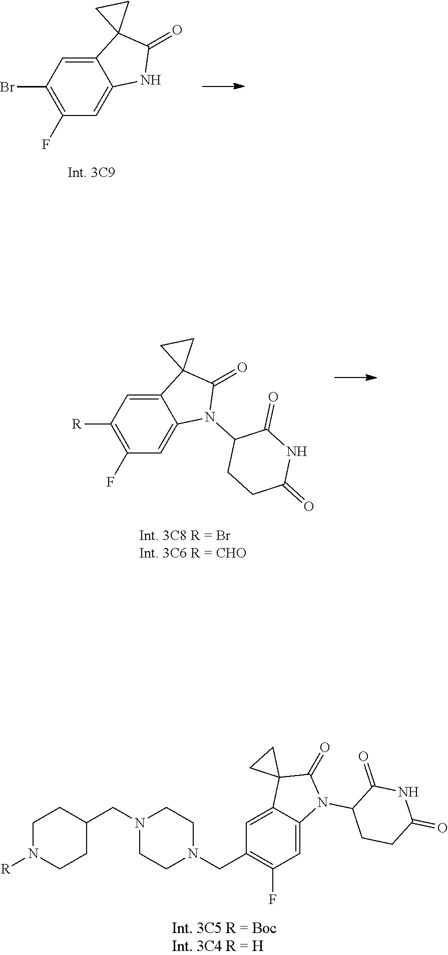

NBS (18.8 g) was added to a solution of 6′-fluorospiro[cyclopropane-1,3′-indolin]-2′-one (17 g) in ACN (340 mL) and stirred for 16 h at RT. The OL (water/EtOAc) was dried, concentrated and washed with Et₂O to give Int. 3C9 5′-bromo-6′-fluorospiro[cyclopropane-1,3′-indolin]-2′-one (15 g). NaH (21 g) was added to a solution of Int. 3C9 (14 g) in THF (0.6 L) at 0° C. 3-Bromopiperidine-2,6-dione (52.5 g) was added and the mixture was stirred 6 h at 60° C. The OL (water/EtOAc) was concentrated, and triturated in PE/MTBE (1:1) to give Int. 3C8 3-(5′-bromo-6′-fluoro-2′-oxospiro-[cyclopropane-1,3′-indolin]-1′-yl)piperidine-2,6-dione (15 g). Pd(OAc)₂ (0.92 g), Int. LL22 (15 g), P(o-tol)₃ (3.7 g), and tert-butyl acrylate (60 mL) were stirred 16 h in DMF (200 mL) and Et₃N (73 mL) at 110° C. under an inert atmosphere. The mixture was filtered. The OL (water/EtOAc) was concentrated and washed with Et₂O to give Int. 3C7 tert-butyl (E)-3-(1′-(2,6-dioxopiperidin-3-yl)-6′-fluoro-2′-oxospiro-[cyclopropane-1,3′-indolin]-5′-yl)acrylate (9 g). OsO₄ (0.11 g), NaIO₄ (14 g), and Int. 3C7 (9 g) were stirred 16 h in THF/H₂O (1:1, 180 mL). The mixture diluted with sat. aq. Na₂S₂O₃ and NaHCO₃ to precipitate a solid that was washed with Et₂O to give Int. 3C6 1′-(2,6-dioxopiperidin-3-yl)-6′-fluoro-2′-oxospiro[cyclopropane-1,3′-indoline]-5′-carbaldehyde (4.5 g). Int. 3C6 (3.5 g) and tert-butyl 4-(piperazin-1-ylmethyl)piperidine-1-carboxylate (6.9 g) were stirred 24 h in DMSO/DIPEA (5:1, 42 mL). STAB (9.4 g) was added at 0° C. and stirring continued for 16 h. The OL (water/EtOAc) was dried, concentrated, and purified by FC (DCM/MeOH 1:0 to 19:1) to give Int. 3C5 tert-butyl 4-((4-((1′-(2,6-dioxopiperidin-3-yl)-6′-fluoro-2′-oxospiro[cyclo-propane-1,3′-indolin]-5′-yl)methyl)-piperazin-1-yl)methyl)piperidine-1-carboxylate (4 g). Int. 3C5 (0.50 g) was stirred 16 h in DCM/TFA (5:2, 7 mL), concentrated, and washed with DCM/Et₂O (1:1) to give Int. 3C4 3-(6′-fluoro-2′-oxo-5′-((4-(piperidin-4-ylmethyl)piperazin-1-yl)methyl)spiro[cyclopropane-1,3′-indolin]-1′-yl)piperidine-2,6-dion (0.30 g, TFA salt).

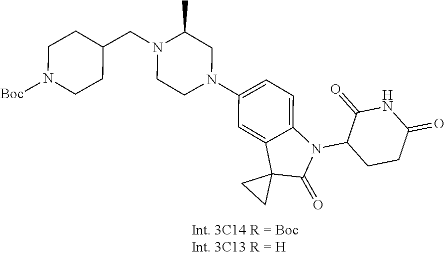 [SMILES: C[C@H]1CN(c2ccc3c(c2)C2(CC2)C(=O)N3C2CCC(=O)NC2=O)CCN1CC1CCN(C(=O)OC(C)(C)C)CC1]

RuPhos (0.98 g) and RuPhos Pd-G4 (0.89 g) were added to a mixture of Int. 3E74 (4.1 g) and 5′-bromo-spiro[cyclopropane-1,3′-indolin]-2′-one (2.5 g) in toluene (30 mL) under an inert atmosphere. 1.0 M LiHMDs in THF (42 mL) was added at 0° C. and the mixture stirred 3 h at 110° C. The OL (aq. NH₄Cl/EtOAc) was dried, concentrated, and purified by FC (PE/EtOAc 1:0 to 0:1) to give Int. 3C14 tert-butyl 4-(((2S)-4-(1′-(2,6-dioxopiperidin-3-yl)-2′-oxospiro[cyclopropane-1,3′-indolin]-5′-yl)-2-methylpiperazin-1-yl)methyl)-piperidine-1-carboxylate (2.4 g). Int. 3C14 (1.1 g) was dissolved in DCM/TFA (27:1, 21 mL) at 0° C. and stirred 16 h at 0° C. to RT, concentrated, and washed with Et₂O to give Int. 3C13 3-(5′-((S)-3-methyl-4-(piperidin-4-ylmethyl)piperazin-1-yl)-2′-oxospiro[cyclopropane-1,3′-indolin]-1′-yl)piperidine-2,6-dione (0.78 g, TFA salt).

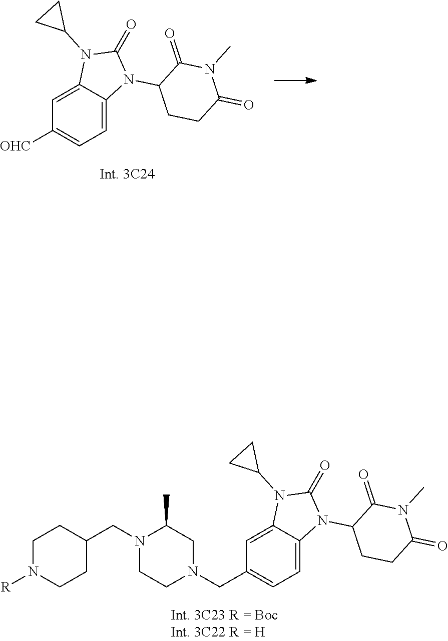

Int. 3E211 (0.50 g) and K₂CO₃ were stirred 16 h in ACN (15 mL) and MeI (0.28 mL) at 10° C. to RT under an inert atmosphere. The OL (water/EtOAc) was dried, concentrated, and purified by FC (DCM/MeOH 1:0 to 9:1) to give Int. 3C24 3-cyclopropyl-1-(1-methyl-2,6-dioxopiperidin-3-yl)-2-oxo-2,3-dihydro-1H-benzo[d]-imidazole-5-carbaldehyde (0.40 g). Ints. 3E74/3C24 (0.53 g/0.30 g) were stirred 16 h in DMSO/DIPEA (13:2, 6.9 mL) under an inert atmosphere. STAB (0.76 g) was added and stirring continued for 8 h. The OL (water/EtOAc) was dried, concentrated, and purified by FC (DCM/MeOH 1:0 to 9:1) to give Int. 3C23 tert-butyl 4-(((2S)-4-((3-cyclopropyl-1-(1-methyl-2,6-dioxo-piperidin-3-yl)-2-oxo-2,3-dihydro-1H-benzo[d]-imidazol-5-yl)methyl)-2-methylpiperazin-1-yl)methyl)-piperidine-1-carboxylate (0.54 g). Int. 3C23 (0.60 g) was stirred 6 h in DCM/4M HCl in dioxane (5:2, 7 mL) at 0° C. The residue after concentration was washed with Et₂O to give Int. 3C22 3-(3-cyclopropyl-5-(((S)-3-methyl-4-(piperidin-4-ylmethyl)piperazin-1-yl)methyl)-2-oxo-2,3-dihydro-1H-benzo[d]imidazol-1-yl)-1-methylpiperidine-2,6-dione (0.45 g, HCl salt).

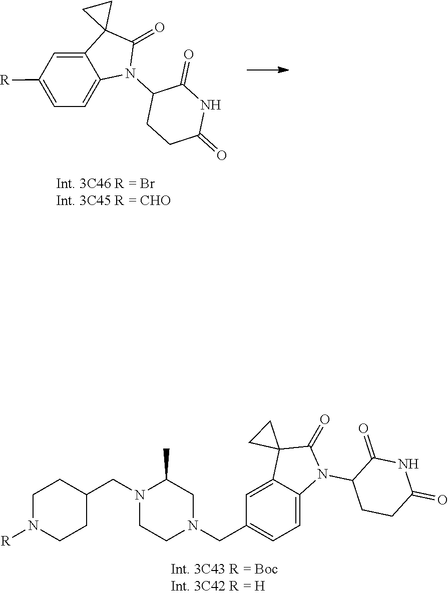

NaH (14.5 g) was added to a solution of 5′-bromospiro[cyclopropane-1,3′-indolin]-2′-one (9 g) in THF (270 mL) at 0° C. 3-Bromopiperidine-2,6-dione (36 g) was added and the mixture stirred 6 h at 60° C. The OL (water/EtOAc) was concentrated and triturated in PE/MTBE (1:1) to give Int. 3C46 3-(5′-bromo-2′-oxospiro-[cyclopropane-1,3′-indolin]-1′-yl)piperidine-2,6-dione (8 g). Et₃N (7.5 mL), Pd(OAc)₂ (0.48 g) and tert-butyl acrylate (32 mL) were added to a solution of Int. 3C46 (7.5 g) and P(o-tol)₃ (2.0 g) in DMF (70 mL) and stirred 16 h at 110° C. under an inert atmosphere. The mixture was filtered. The OL (water/EtOAc) was concentrated and washed with PE to give Int. 3C45 tert-butyl (E)-3-(1′-(2,6-dioxopiperidin-3-yl)-2′-oxospiro-[cyclopropane-1,3′-indolin]-5′-yl)acrylate (5 g). OsO₄ (64 mg), NaIO₄ (8.1 g), and Int. 3C45 (5.0 g) were stirred 2 h in THF/H₂O (1:1, 100 mL). The mixture was diluted with sat. aq. Na₂S₂O₃·5H₂O to precipitate Int. 3C44 1′-(2,6-dioxo-piperidin-3-yl)-2′-oxospiro-[cyclopropane-1,3′-indoline]-5′-carbaldehyde (3.0 g). Ints. 3E74/3C44 (1.5 g/2.5 g) were stirred 24 h in DMSO/DIPEA (1:1, 10.5 mL). STAB (7.1 g) was added at 0° C. and stirring was continued for 16 h at 0° C. to RT. The OL (water/EtOAc) was dried, concentrated, and purified by FC (DCM/MeOH 1:0 to 19:1) to give Int. 3C43 tert-butyl 4-(((2S)-4-((1′-(2,6-dioxopiperidin-3-yl)-2′-oxospiro-[cyclopropane-1,3′-indolin]-5′-yl)methyl)-2-methylpiperazin-1-yl)methyl)piperidine-1-carboxylate (0.4 g). Int. 3C43 (1.0 g) was stirred 4 h in DCM/4M HCl in dioxane (7:1, 23 mL) at 0° C. to RT, concentrated, and washed with Et₂O to give Int. 3C42 3-(5′-(((S)-3-methyl-4-(piperidin-4-ylmethyl)-piperazin-1-yl)-methyl)-2′-oxospiro[cyclopropane-1,3′-indolin]-1′-yl)piperidine-2,6-dione (0.80 g, HCl salt).

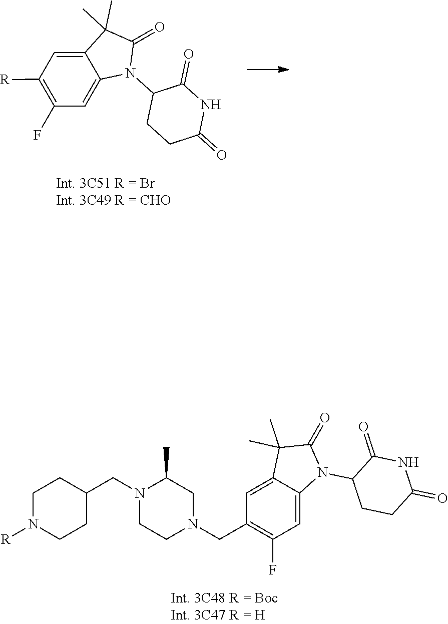

NaH (15.5 g) was added to a solution of 5-bromo-6-fluoro-3,3-dimethylindolin-2-one (10 g) in THF (200 mL) at 0° C. and stirred for 15 min at RT. 3-Bromopiperidine-2,6-dione (37 g) was added and the mixture was stirred 16 h at 70° C. The OL (water/EtOAc) was dried, concentrated, and washed with hexane/EtOAc (2:1) to give Int. 3C51 3-(5-bromo-6-fluoro-3,3-dimethyl-2-oxoindolin-1-yl)piperidine-2,6-dione (7.5 g). Pd(OAc)₂ (0.61 g), P(o-tol)₃ (2.5 g), and Int. 3C51 (10 g) were stirred 16 h in DMF/Et₃N (2:1, 148 mL) and tert-butyl acrylate (40 mL) at 110° C. under an inert atmosphere. The mixture was filtered. The OL (water/EtOAc) was concentrated and washed with Et₂O to give Int. 3C50 tert-butyl (E)-3-(1-(2,6-dioxopiperidin-3-yl)-6-fluoro-3,3-dimethyl-2-oxoindolin-5-yl)acrylate (7 g). OsO₄ (67 mg) and NaIO₄ (7.0 g) were added to a solution of Int. 3C50 (5.5 g) in THF/H₂O (1:1, 60 mL) and stirred 12 h. The mixture diluted with sat. aq. Na₂S₂O₃·5H₂O to precipitate Int. 3C49 1-(2,6-dioxopiperidin-3-yl)-6-fluoro-3,3-dimethyl-2-oxoindoline-5-carbaldehyde (4.0 g). Ints. 3E74/3C49 (4.5 g/4.0 g) were stirred 24 h in DMSO/DIPEA (4:1, 49 mL). STAB (11 g) was added at 0° C. and stirring continued for 16 h at RT. The OL (water/EtOAc) was dried, and concentrated to give Int. 3C48 tert-butyl 4-(((2S)-4-((1-(2,6-dioxopiperidin-3-yl)-6-fluoro-3,3-dimethyl-2-oxoindolin-5-yl)-methyl)-2-methyl-piperazin-1-yl)methyl)piperidine-1-carboxylate (2.5 g). Int. 3C48 (1.8 g) was mixed in DCM/4M HCl in dioxane (2:1, 15 mL) at 0° C. and was stirred for 5 h at RT, concentrated, and washed with Et₂O to give Int. 3C47 3-(6-fluoro-3,3-dimethyl-5-(((S)-3-methyl-4-(piperidin-4-yl-methyl)piperazin-1-yl)methyl)-2-oxoindolin-1-yl)piperidine-2,6-dione (1.2 g, HCl salt).

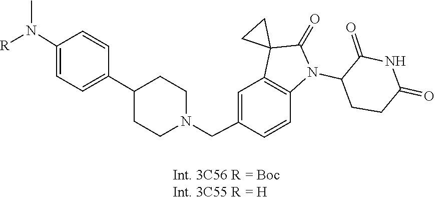

Ints. 3E266/3C44 (2.0 g/1.8 g) were stirred in DMSO/DIPEA (4:1, 19 mL) for 24 h at RT. STAB (4.9 g) was added and the mixture was stirred 16 h at 0° C. to RT. The OL (water/EtOAc) was dried, concentrated, and purified by FC (DCM/MeOH 1:0 to 14:1) to give Int. 3C56 tert-butyl (4-(1-((1′-(2,6-dioxopiperidin-3-yl)-2′-oxospiro[cyclopropane-1,3′-indolin]-5′-yl)methyl)piperidin-4-yl)phenyl)-(methyl)carbamate (1.3 g). Int. 3C56 (1.3 g) was stirred 16 h in DCM/TFA (4:1, 25 mL) at 0° C. to RT, concentrated and washed with Et₂O to give Int. 3C55 3-(5′-((4-(4-(methylamino)phenyl)piperidin-1-yl)methyl)-2′-oxospiro[cyclopropane-1,3′-indolin]-1′-yl)piperidine-2,6-dione (1.2 g, TFA salt).

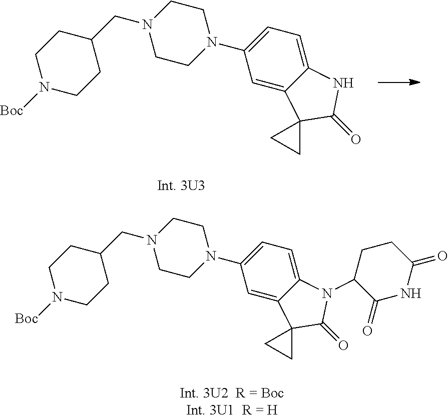 [SMILES: CC(C)(C)OC(=O)N1CCC(CN2CCN(c3ccc4c(c3)C3(CC3)C(=O)N4)CC2)CC1 ; CC(C)(C)OC(=O)N1CCC(CN2CCN(c3ccc4c(c3)C3(CC3)C(=O)N4C3CCC(=O)NC3=O)CC2)CC1]

5′-Bromospiro[cyclopropane-1,3′-indolin]-2′-one (3 g), tert-butyl 4-(piperazin-1-ylmethyl)piperidine-1-carboxylate (7.1 g), RuPhos (1.2 g), and RuPhos Pd-G4 (1.1 g) were stirred 3 h in toluene (35 mL) and 1M LiHMDs in THF (63 mL) at 70° C. under an inert atmosphere. The OL (water/EtOAc) was dried, concentrated, and purified by FC (PE/EtOAc 1:0 to 1:4) to give Int. 3U3 tert-butyl 4-((4-(2′-oxospiro-[cyclopropane-1,3′-indolin]-5′-yl)piperidin-1-yl)methyl)-piperidine-1-carboxylate (2.5 g). NaH (1.4 g) and 3-bromopiperidine-2,6-dione (3.3 g) were added to a mixture of Int. 3U3 (1.5 g) in DMF (15 mL) and stirred for 48 h at 60° C. The OL (water/EtOAc) was dried, concentrated, and purified by FC (DCM/MeOH 19:1) to give Int. 3U2 tert-butyl 4-((4-(1′-(2,6-dioxopiperidin-3-yl)-2′-oxospiro-[cyclopropane-1,3′-indolin]-5′-yl)piperazin-1-yl)methyl)piperidine-1-carboxylate (1.3 g). Int. 2U2 (1.5 g) was stirred 12 h in DCM/TFA (2:1, 22 mL) at 0° C. to RT. The mixture was concentrated and washed with Et₂O to give Int. 3U1 3-(2′-oxo-5′-(4-(piperidin-4-ylmethyl)piperazin-1-yl)spiro[cyclopropane-1,3′-indolin]-1′-yl)piperidine-2,6-dione (1.3 g, TFA salt).

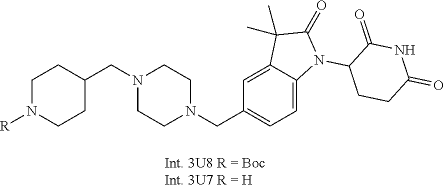

Int. 3E293 (1 g) and tert-butyl 4-(piperazin-1-ylmethyl)piperidine-1-carboxylate (1.9 g) were stirred 24 h in DMSO/DIPEA (15/2.3 mL). STAB (2.8 g) was added and stirring continued 12 h at 0° C. to RT. The OL (water/DCM/MeOH) was dried, concentrated, and purified by FC (DCM/MeOH 1:0 to 19:1) to give Int. 3U8 tert-butyl 4-((4-((1-(2,6-dioxopiperidin-3-yl)-3,3-dimethyl-2-oxoindolin-5-yl)-methyl)-piperazin-1-yl)methyl)piperidine-1-carboxylate (0.80 g). Int. 3U8 (0.80 g) was stirred 5 h in DCM/4M HCl in dioxane (3:2, 10 mL) at 0° C. to RT and concentrated to give Int. 3U7 3-(3,3-dimethyl-2-oxo-5-((4-(piperidin-4-ylmethyl)piperazin-1-yl)methyl)indolin-1-yl)-piperidine-2,6-dione (0.50 g, HCl salt).

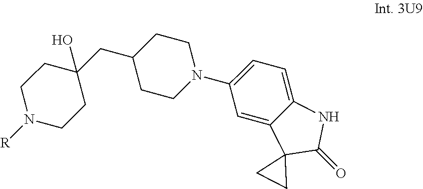

Int. 3U10 (4.5 g), 5′-bromospiro[cyclopropane-1,3′-indolin]-2′-one (3 g), RuPhos (0.59 g), and RuPhos Pd-G4 (1.1 g) were stirred 2 h in toluene/1M LiHMDS in THF (30/63 mL) at 0° C. to 85° C. The OL (water/EtOAc) was dried, concentrated, and purified by FC (PE/EtOAc 1:1 to 1:4) to give Int. 3U9 tert-butyl 4-hydroxy-4-((1-(2′-oxospiro[cyclopropane-1,3′-indolin]-5′-yl)piperidin-4-yl)-methyl)piperidine-1-carboxylate (1 g).

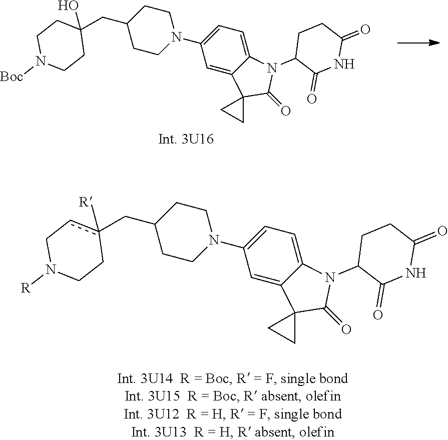

NaH (1.1 g) and Int. 3U9 (2 g) were stirred 0.5 h in THF (50 mL) at 0° C. 3-Bromopiperidine-2,6-dione (3.0 g) was added portion-wise and stirring continued 5 h at 0-75° C. The OL (water/EtOAc) was dried, concentrated, and washed with pentane/Et₂O (1:1) to give Int. 3U16 tert-butyl 4-((1-(1′-(2,6-dioxopiperidin-3-yl)-2′-oxospiro[cyclopropane-1,3′-indolin]-5′-yl)-piperidin-4-yl)methyl)-4-hydroxy-piperidine-1-carboxylate (1 g). Int. 3U16 (0.70 g) was stirred 1 h in DCM (10 mL) and DAST (0.20 mL) in DCM (5 mL) at −78° C. The OL (sat. aq. NaHCO₃/DCM) was dried and concentrated to give Int. 3U14 and 3U15 tert-butyl 4-((1-(1′-(2,6-dioxopiperidin-3-yl)-2′-oxospiro-[cyclopropane-1,3′-indolin]-5′-yl)piperidin-4-yl)methyl)-4-fluoro-piperidine-1-carboxylate and tert-butyl 4-((1-(1′-(2,6-dioxo-piperidin-3-yl)-2′-oxospiro[cyclopropane-1,3′-indolin]-5′-yl)piperidin-4-yl)-methyl)-3,6-dihydro-pyridine-1 (2H)-carboxylate mixture (0.50 g). Int. 3U14/3U15 mixture (0.60 g) was stirred 3 h in DCM/TFA (2:1, 7.5 mL). The mixture was concentrated and washed with Et₂O to give Int. 3U12 and 3U13 3-(5′-(4-((4-fluoropiperidin-4-yl)methyl)piperidin-1-yl)-2′-oxospiro[cyclopropane-1,3′-indolin]-1′-yl)piperidine-2,6-dione and 3-(2′-oxo-5′-(4-((1,2,3,6-tetrahydropyridin-4-yl)methyl)-piperidin-1-yl)spiro[cyclopropane-1,3′-indolin]-1′-yl)piperidine-2,6-dione mixture (0.50 g).

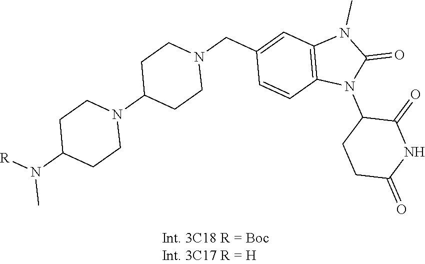

Ints. 3U19/3U67 (0.15 g/0.18 g) and 4 Å MS were stirred in DCE (2 mL) ON at RT. NaCNBH₃ (41 mg) was added and stirring continued for 3 h. The mixture was concentrated and HPLC-purified to give Int. 3U18 tert-butyl (1′-((1-(2,6-dioxopiperidin-3-yl)-3-methyl-2-oxo-2,3-dihydro-1H-benzo[d]imidazol-5-yl)methyl)-[1,4′-bipiperidin]-4-yl)(methyl)carbamate (74 mg). Int. 3U18 (74 mg) was stirred 20 h in DCM/TFA (10:1, 1.1 mL). The mixture was concentrated to give Int. 3U17 3-(3-methyl-5-((4-(methylamino)-[1,4′-bipiperidin]-1′-yl)methyl)-2-oxo-2,3-dihydro-1H-benzo[d]imidazol-1-yl)piperidine-2,6-dione (70 mg, TFA salt).

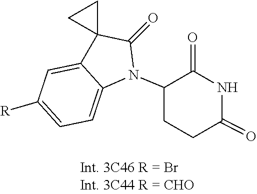

Int. 3C44. 1′-(2,6-dioxo-piperidin-3-yl)-2′-oxospiro[cyclopropane-1,3′-indoline]-5′-carbaldehyde

NaH (14.5 g) was added to a solution of 5′-bromospiro[cyclopropane-1,3′-indolin]-2′-one (9 g) in THF (270 mL) at 0° C. 3-Bromopiperidine-2,6-dione (36 g) was added and the mixture stirred 6 h at 60° C. The OL (water/EtOAc) was concentrated and triturated in PE/MTBE (1:1) to give Int. 3C46 3-(5′-bromo-2′-oxospiro-[cyclopropane-1,3′-indolin]-1′-yl)piperidine-2,6-dione (8 g). Et₃N (7.5 mL), Pd(OAc)₂ (0.48 g) and tert-butyl acrylate (32 mL) were added to a solution of Int. 3C46 (7.5 g) and P(o-tol)₃ (2.0 g) in DMF (70 mL) and stirred 16 h at 110° C. under an inert atmosphere. The mixture was filtered. The OL (water/EtOAc) was concentrated and washed with PE to give Int. 3C45 tert-butyl (E)-3-(1′-(2,6-dioxopiperidin-3-yl)-2′-oxospiro-[cyclopropane-1,3′-indolin]-5′-yl)acrylate (5 g). OsO₄ (64 mg), NaIO₄ (8.1 g), and Int. 3C45 (5.0 g) were stirred 2 h in THF/H₂O (1:1, 100 mL). The mixture was diluted with sat. aq. Na₂S₂O₃·5H₂O to precipitate Int. 3C44 1′-(2,6-dioxo-piperidin-3-yl)-2′-oxospiro[cyclopropane-1,3′-indoline]-5′-carbaldehyde (3.0 g).

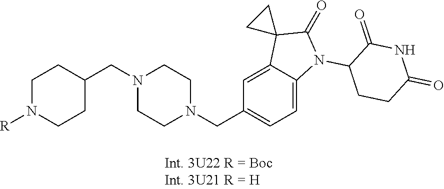

Int. 3C44 (8 g) and tert-butyl 4-(piperazin-1-ylmethyl)piperidine-1-carboxylate (12 g) were stirred 24 h in DMSO (50 mL) and DIPEA (9.8 mL). STAB (15 g) was added and stirring continued 16 h at 0° C. to RT. The OL (water/EtOAc) was dried, concentrated, and purified by FC (DCM/MeOH 1:0 to 19:1) to give Int. 3U22 tert-butyl 4-((4-((1′-(2,6-dioxopiperidin-3-yl)-2′-oxospiro[cyclopropane-1,3′-indolin]-5′-yl)methyl)piperazin-1-yl)methyl)piperidine-1-carboxylate (2.3 g). Int. 3U22 (0.50 g) was stirred 5 h in DCM/TFA (5:1, 6 mL) at 0° C. to RT. The mixture was concentrated and triturated in Et₂O to give Int. 3U21 3-(2′-oxo-5′-((4-(piperidin-4-ylmethyl)piperazin-1-yl)methyl)spiro[cyclopropane-1,3′-indolin]-1′-yl)piperidine-2,6-dione (0.30 g, TFA salt).

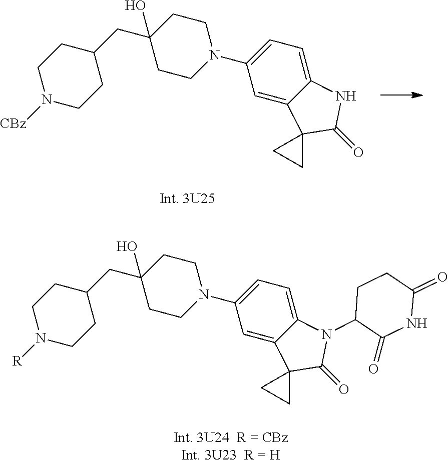

RuPhos (1.8 g), RuPhos Pd-G4 (1.6 g), Int. 3U26 (7.7 g), and 5′-bromospiro[cyclopropane-1,3′-indolin]-2′-one (4.6 g) were stirred 5 h in toluene/THF (1:1, 200 mL) and 1M LiHMDs in THF (97 mL) at 0-80° C. under an inert atmosphere. The OL (water/EtOAc) was dried, and concentrated to give Int. 3U25 benzyl 4-((4-hydroxy-1-(2′-oxospiro[cyclopropane-1,3′-indolin]-5′-yl)piperidin-4-yl)methyl)piperidine-1-carboxylate (3 g). NaH (1.5 g) was added to a solution of Int. 3U25 (1.5 g) in THF (150 mL) and stirred for 0.5 h at 0° C. 3-Bromopiperidine-2,6-dione (4.1 g) was added and stirring continued for 2 h at 75° C. under an inert atmosphere. The OL (water/EtOAc) was dried, concentrated, and washed with Et₂O/pentane (1:1) to give Int. 3U24 (0.60 g). AlCl₃ (68 mg) and Int. 3U24 benzyl 4-((1-(1′-(2,6-dioxopiperidin-3-yl)-2′-oxospiro-[cyclopropane-1,3′-indolin]-5′-yl)-4-hydroxypiperidin-4-yl)methyl)piperidine-1-carboxylate (0.10 g) were stirred 16 h in HFP (5 mL) under an inert atmosphere. The mixture was concentrated and washed with Et₂O to give Int. 3U23 3-(5′-(4-hydroxy-4-(piperidin-4-ylmethyl)piperidin-1-yl)-2′-oxospiro[cyclopropane-1,3′-indolin]-1′-yl)piperidine-2,6-dione (0.15 g).

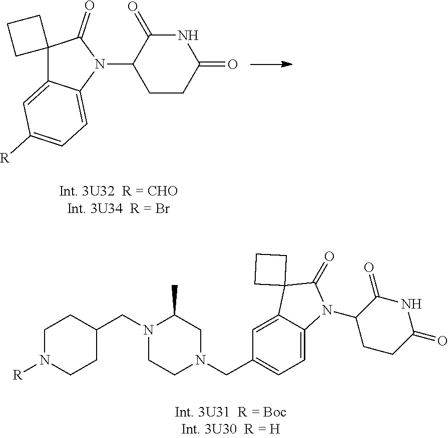

NaH (48 g) was added portion-wise to a solution of 5′-bromospiro[cyclobutane-1,3′-indolin]-2′-one (30 g) in THF (500 mL) at 0° C. and stirred for 30 min. 3-Bromopiperidine-2,6-dione (30 g) was added and stirring continued 16 h at 0° C. to 65° C. The OL (aq. NH₄Cl/EtOAc) was dried, concentrated, and triturated in Et₂O to give Int. 3U34 3-(5′-bromo-2′-oxospiro[cyclobutane-1,3′-indolin]-1′-yl)-piperidine-2,6-dione (25 g). Pd(OAc)₂ (0.82 g), P(o-tol)₃ (4.4 g), and Int. 3U34 (22 g) were stirred 16 h in DMF/Et₃N (25:3, 280 mL) and tert-butyl acrylate (27 mL) at 110° C. under an inert atmosphere and filtered. The OL (water/EtOAc) was dried, concentrated, and purified by FC (PE/EtOAc 1:1) to give Int. 3U33 tert-butyl (E)-3-(1′-(2,6-dioxopiperidin-3-yl)-2′-oxospiro[cyclobutane-1,3′-indolin]-5′-yl)acrylate (13 g). NaIO₄ (16 g) and OsO₄ (0.74 g) were added to a solution of Int. 3U33 (12 g) in THF/H₂O (4:3, 140 mL). The mixture was stirred 16 h. The OL (sat. aq. Na₂S₂O₃/EtOAc) was dried, concentrated, and triturated in Et₂O to give Int. 3U32 1′-(2,6-dioxopiperidin-3-yl)-2′-oxospiro-[cyclobutane-1,3′-indoline]-5′-carbaldehyde (7.0 g). Ints. 3E74/3U32 (6.9 g/4.0 g) were stirred 16 h in DIPEA (14 mL) and DMSO (50 mL). STAB (11 g) was added and stirring continued 16 h at 0° C. to RT. The OL (water/EtOAc) was dried, concentrated, and purified by FC (DCM/MeOH 24:1) to give Int. 3U31 tert-butyl 4-(((2S)-4-((1′-(2,6-dioxopiperidin-3-yl)-2′-oxospiro[cyclobutane-1,3′-indolin]-5′-yl)methyl)-2-methylpiperazin-1-yl)methyl)piperidine-1-carboxylate (4.5 g). Int. 3U31 (4.5 g) was stirred 6 h in DCM/4M HCl in dioxane (5:2, 70 mL) at 0° C. to RT. The mixture was concentrated and washed with Et₂O to give Int. 3U30 3-(5′-(((S)-3-methyl-4-(piperidin-4-ylmethyl)piperazin-1-yl)methyl)-2′-oxospiro[cyclobutane-1,3′-indolin]-1′-yl)-piperidine-2,6-dione (3.5 g, HCl salt).

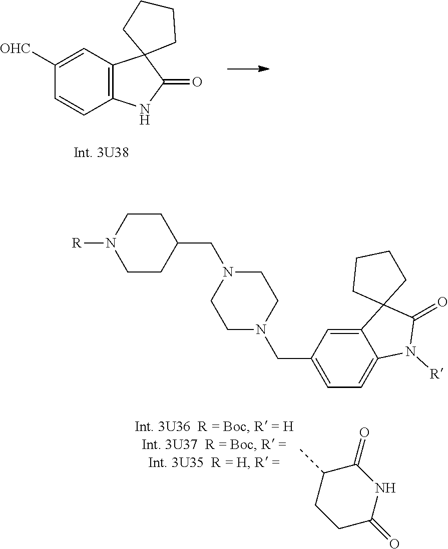

PdDPPFCl₂-DCM (1.5 g) and 5′-bromospiro[cyclopentane-1,3′-indolin]-2′-one (5 g) were stirred 16 h in DMF/Et₃N (19:2, 55 mL) and Et₃SiH (9 mL) at 130° C. under 440 psi of CO. The OL (water/EtOAc) was dried, concentrated, and purified by FC (PE/MTBE 3:2 to 1:2) to give Int. 3U38 2′-oxospiro-[cyclopentane-1,3′-indoline]-5′-carbaldehyde (2.8 g). tert-Butyl 4-(piperazin-1-ylmethyl)piperidine-1-carboxylate (1.4 g) and Int. 3U38 (1 g) were stirred 0.3 h in DCM/AcOH (56:1, 30.5 mL). STAB (3.0 g) was added and stirring continued for 11 h. The OL (sat. aq. NaHCO₃/EtOAc) was concentrated and purified by FC (MTBE/MeOH 1:0 to 19:1) to give Int. 3U37 tert-butyl 4-((4-((2′-oxospiro-[cyclopentane-1,3′-indolin]-5′-yl)methyl)piperazin-1-yl)methyl)piperidine-1-carboxylate (2 g). NaH (1.0 g) was added to a solution of Int. 3U37 (2 g) in THF (50 mL) and stirred for 20 min at RT. 3-Bromopiperidine-2,6-dione (4.0 g) was added and stirring continued for 14 h at 60° C. The OL (aq. NH₄Cl/EtOAc) was concentrated and purified by FC (MTBE/MeOH) to give Int. 3U36 tert-butyl 4-((4-((1′-(2,6-dioxo-piperidin-3-yl)-2′-oxospiro[cyclopentane-1,3′-indolin]-5′-yl)methyl)-piperazin-1-yl)-methyl)piperidine-1-carboxylate (1 g). Int. 3U36 (0.5 g) was stirred 14 h in MeOH/4M HCl in dioxane (1:2, 30 mL). The mixture was concentrated to give Int. 3U35 3-(2′-oxo-5′-((4-(piperidin-4-yl-methyl)-piperazin-1-yl)methyl)spiro[cyclopentane-1,3′-indolin]-1′-yl)piperidine-2,6-dione (0.6 g, HCl salt).

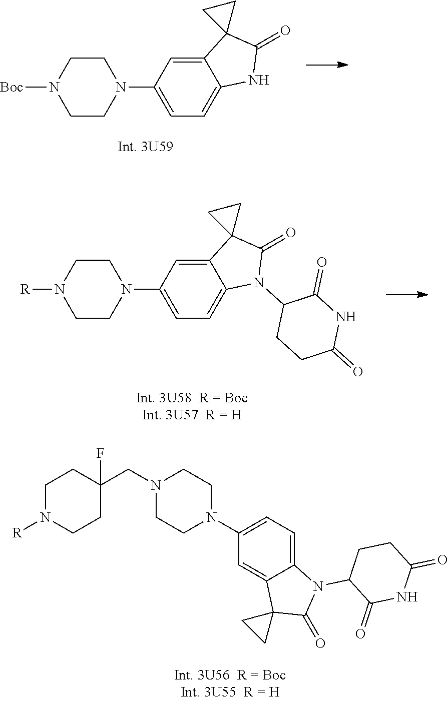

RuPhos (0.59 g), RuPhos Pd-G4 (0.54 g), and tert-butyl piperazine-1-carboxylate (1.4 g), 5′-bromospiro[cyclopropane-1,3′-indolin]-2′-one (1.5 g) were stirred 16 h in toluene (20 mL) and 1M LiHMDs in THF (25 mL) at 110° C. under an inert atmosphere. The OL (water/EtOAc) was dried, concentrated, and purified by FC (hexane/EtOAc 3:7) to give Int. 3U59 tert-butyl 4-(2′-oxospiro-[cyclopropane-1,3′-indolin]-5′-yl)piperazine-1-carboxylate (1.2 g). NaH (1.7 g) and 3-bromo-piperidine-2,6-dione (4.7 g) were added to a mixture of Int. 3U59 (1.2 g) in DMF (20 mL) and stirred for 16 h at 70° C. The OL (water/EtOAc) was dried, concentrated, and triturated in Et₂O to give Int. 3U58 tert-butyl 4-(1′-(2,6-dioxopiperidin-3-yl)-2′-oxospiro-[cyclopropane-1,3′-indolin]-5′-yl)-piperazine-1-carboxylate (1 g). Int. 3U58 (1 g) was stirred 16 h in DCM/TFA (2:1, 15 mL) at 0° C. to RT. The mixture was concentrated and triturated in Et₂O to give Int. 3U57 3-(2′-oxo-5′-(piperazin-1-yl)-spiro[cyclopropane-1,3′-indolin]-1′-yl)piperidine-2,6-dione (0.90 g, TFA salt). Int. 3U57 (1.2 g) and tert-butyl 4-fluoro-4-formylpiperidine-1-carboxylate (0.60 g) in were stirred 16 h in DMSO (10 mL) and DIPEA (2.2 mL) 0° C. to RT. STAB (2.1 g) was added and stirring continued 16 h at 0° C. to RT. The OL (water/EtOAc) was dried, concentrated, and purified by FC (DCM/MeOH 19:1) to give Int. 3U56 tert-butyl 4-((4-(1′-(2,6-dioxopiperidin-3-yl)-2′-oxospiro[cyclopropane-1,3′-indolin]-5′-yl)-piperazin-1-yl)methyl)-4-fluoropiperidine-1-carboxylate (0.60 g). Int. 3U56 (0.8 g) was stirred 16 h in DCM/TFA (20:1, 10.5 mL) at 0° C. to RT. The mixture was concentrated and triturated in Et₂O to give Int. 3U55 3-(5′-(4-((4-fluoropiperidin-4-yl)methyl)piperazin-1-yl)-2′-oxospiro[cyclopropane-1,3′-indolin]-1′-yl)piperidine-2,6-dione (0.75 g, TFA salt).

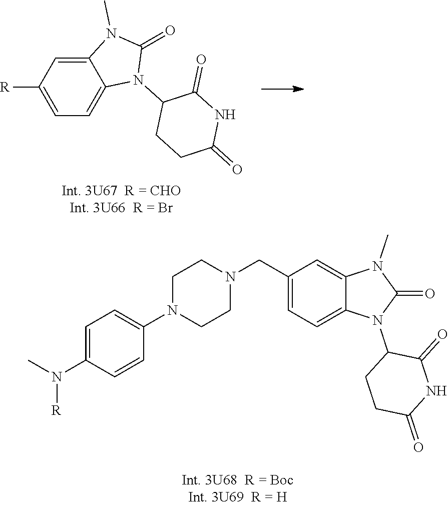

Pd(OAc)₂ (1.7 g), Int. 3B3 (25 g), and P(o-tol)₃ (4.5 g) were stirred 16 h in DMF/Et₃N (7:3, 172 mL) and tert-butyl acrylate (54 mL) at 110° C. under an inert atmosphere. The mixture was filtered. The OL (water/EtOAc) was concentrated, and triturated in pentane to give tert-butyl (E)-3-(1-(2,6-dioxopiperidin-3-yl)-3-methyl-2-oxo-2,3-dihydro-1H-benzo[d]imidazol-5-yl)acrylate (17 g). NaIO₄ (56 g) and OsO₄ (2.2 g) and were added to a solution of Int. tert-butyl (E)-3-(1-(2,6-dioxopiperidin-3-yl)-3-methyl-2-oxo-2,3-dihydro-1H-benzo[d]-imidazol-5-yl)acrylate (34 g) in THF/H₂O (1:1, 100 mL) and stirred for 2 h at RT. The mixture diluted with sat. aq. Na₂S₂O₃·5H₂O and stirred for 1 h to precipitate a solid that was washed with Et₂O to give Int. 3U67 1-(2,6-dioxopiperidin-3-yl)-3-methyl-2-oxo-2,3-dihydro-1H-benzo[d]imidazole-5-carbaldehyde (14 g). Ints. 3E266/3U67 (3.4 g/2.0 g) were stirred 24 h in DMSO/DIPEA (10:3, 26 mL). STAB (5.9 g) was added and stirring continued 16 h at 0° C. to RT. The OL (water/DCM/MeOH) was dried, concentrated, and purified by FC (DCM/MeOH 19:1 to 10:1) to give Int. 3U68 tert-butyl (4-(1-((1-(2,6-dioxopiperidin-3-yl)-3-methyl-2-oxo-2,3-dihydro-1H-benzo[d]imidazol-5-yl)methyl)piperidin-4-yl)phenyl)(methyl)carbamate (1 g). Int. 3U68 (1.2 g) was stirred 5 h in DCM/4M HCl in dioxane (3:2, mL) at 0° C. to RT. The mixture was concentrated and washed with Et₂O to give Int. 3U69 3-(3-methyl-5-((4-(4-(methylamino)phenyl)-piperidin-1-yl)methyl)-2-oxo-2,3-dihydro-1H-benzo[d]imidazol-1-yl)piperidine-2,6-dione (1.0 g, HCl salt).

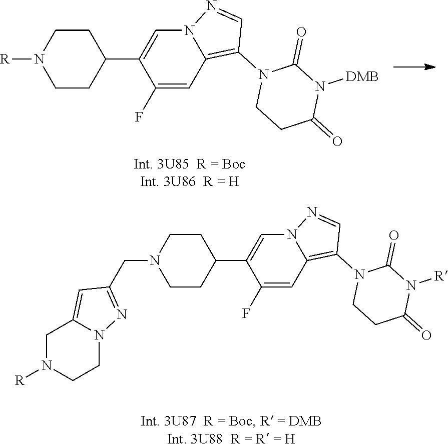

Int. 3U84, (125 mg), CuI (43 mg), 3-(2,4-dimethoxybenzyl)dihydropyrimidine-2,4 (1H,3H)-dione (89 mg), and Cs₂CO₃ (274 mg) were stirred ON in dioxane (4 mL) and trans-N,N′-dimethylcyclohexane-1,2-diamine (71 μL) at 90° C. under an inert atmosphere. The OL (water/EtOAc) was dried, concentrated, and HPLC-purified to give Int. 3U85 tert-butyl 4-(3-(3-(2,4-dimethoxybenzyl)-2,4-dioxotetrahydropyrimidin-1 (2H)-yl)-5-fluoropyrazolo[1,5-a]pyridin-6-yl)piperidine-1-carboxylate (162 mg). Int. 3U85 (162 mg) was stirred 2 h in HFP/HCl 10M (100:1, 3.03 mL). The mixture was concentrated to give Int. 3U86 3-(2,4-dimethoxybenzyl)-1-(5-fluoro-6-(piperidin-4-yl)pyrazolo[1,5-a]pyridin-3-yl)dihydropyrimidine-2,4 (1H,3H)-dione (150 mg, HCl salt). Ints. 2Q11/3U86 (87 mg/150 mg), Et₃N (81 μL) and crushed 4 Å MS were stirred ON in DCE (5 mL). STAB (250 mg) was added and stirring continued for 3 h. The mixture was filtered and concentrated. The OL (water/DCM) was dried, and concentrated to give Int. 3U87 tert-butyl 2-((4-(3-(3-(2,4-dimethoxybenzyl)-2,4-dioxotetrahydro-pyrimidin-1 (2H)-yl)-5-fluoropyrazolo[1,5-a]pyridin-6-yl)piperidin-1-yl)methyl)-6,7-di-hydro-pyrazolo-[1,5-a]pyrazine-5 (4H)-carboxylate (208 mg). Int. 3U87 (208 mg) was stirred 3 days in TFA (5 mL) at 70° C. and concentrated to give Int. 3U88 1-(5-fluoro-6-(1-((4,5,6,7-tetrahydropyrazolo[1,5-a]pyrazin-2-yl)methyl)piperidin-4-yl)pyrazolo[1,5-a]pyridin-3-yl)dihydropyrimidine-2,4 (1H,3H)-dione (0.13 g, TFA salt).

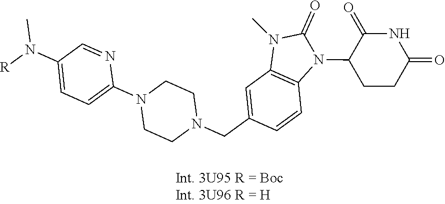

Ints. 3U94/3U67 (4.0 g/3.0 g) were stirred 24 h in DMSO/DIPEA (9:1, 56 mL). STAB (8.9 g) was added and stirring continued 16 h at 0° C. to RT. The OL (water/DCM/MeOH) was dried, concentrated, and purified by FC (DCM/MeOH 19:1 to 9:1) to give Int. 3U95 tert-butyl (6-(4-((1-(2,6-dioxopiperidin-3-yl)-3-methyl-2-oxo-2,3-dihydro-1H-benzo[d]imidazol-5-yl)-methyl)piperazin-1-yl)pyridin-3-yl)(methyl)carbamate (1.5 g). Int. 3U95 (0.50 g) was stirred 5 h in DCM/4M HCl in dioxane (1:1, 10 mL) at 0° C. to RT. The mixture was concentrated and washed with Et₂O/EtOAc to give Int. 3U96 3-(3-methyl-5-((4-(5-(methylamino)pyridin-2-yl)-piperazin-1-yl)methyl)-2-oxo-2,3-dihydro-1H-benzo[d]imidazol-1-yl)piperidine-2,6-dione (0.43 g, HCl salt).

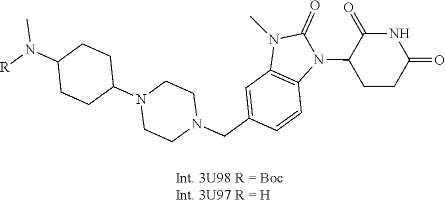

Ints. 3U99/3U67 (0.10 g/0.12 g) and 4 Å MS were stirred ON in DCE (2 mL). NaCNBH₃ (31 mg) was added and stirring continued 3 h. The mixture was filtered, concentrated, and HPLC-purified to give Int. 3U98 tert-butyl (4-(4-((1-(2,6-dioxopiperidin-3-yl)-3-methyl-2-oxo-2,3-dihydro-1H-benzo[d]-imidazol-5-yl)methyl)-piperazin-1-yl)cyclohexyl)(methyl)carbamate (22 mg). Int. 3U98 (22 mg) was stirred 20 h in DCM/TFA (29:1, 1.03 mL). The mixture was concentrated to give Int. 3U97 3-(3-methyl-5-((4-(4-(methylamino)cyclohexyl)-piperazin-1-yl)methyl)-2-oxo-2,3-dihydro-1H-benzo[d]imidazol-1-yl)piperidine-2,6-dione (17 mg, TFA salt).

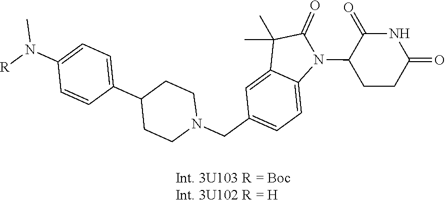

Ints. 3E266/3E293 (2.3 g/2.0 g) were stirred 24 h in DMSO/DIPEA (9:1, 44 mL). STAB (5.6 g) was added and stirring continued 16 h at 0° C. to RT. The OL (water/EtOAc) was dried, concentrated, and purified by FC (DCM/MeOH 1:0 to 14:1) to give Int. 3U103 tert-butyl (4-(1-((1-(2,6-dioxopiperidin-3-yl)-3,3-dimethyl-2-oxoindolin-5-yl)methyl)piperidin-4-yl)phenyl)(methyl)-carbamate (1.8 g). Int. 3U103 (0.90 g) was stirred 16 h in DCM/4M HCl in dioxane (2:1, 15 mL) at 0° C. to RT. The mixture was concentrated and washed with Et₂O to give Int. 3U102 3-(3,3-dimethyl-5-((4-(4-(methylamino)-phenyl)piperidin-1-yl)methyl)-2-oxoindolin-1-yl)piperidine-2,6-dione (0.75 g, HCl salt).

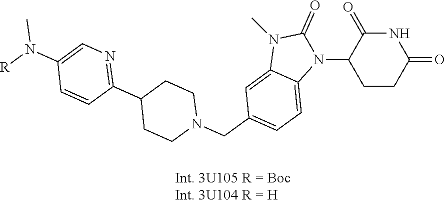

Ints. 3U106/3U67 (1.7 g/1.3 g) were stirred 16 h in DMSO/DIPEA (13:3, 12.3 mL) at 0° C. to RT. STAB (1.8 g) and DCE (10 mL) were added and stirring was continued 16 h at 0-60° C. under an inert atmosphere. The OL (aq. NH₄Cl/EtOAc) was dried, and concentrated to give Int. 3U105 tert-butyl (6-(1-((1-(2,6-dioxopiperidin-3-yl)-3-methyl-2-oxo-2,3-dihydro-1H-benzo[d]-imidazol-5-yl)-methyl)-piperidin-4-yl)pyridin-3-yl)(methyl)-carbamate (1.2 g). Int. 3U105 (0.20 g) was dissolved in DCM/4M HCl in dioxane (3:2, 5 mL) at 0° C. and stirred for 16 h at RT, concentrated and washed with Et₂O to give Int. 3U104 3-(3-methyl-5-((4-(5-(methyl-amino)pyridin-2-yl)piperidin-1-yl)methyl)-2-oxo-2,3-dihydro-1H-benzo[d]imidazol-1-yl)piperidine-2,6-dione (0.14 g, HCl salt).

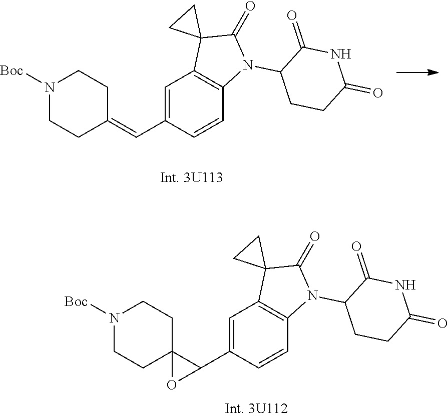 [SMILES: CC(C)(C)OC(=O)N1CCC(=Cc2ccc3c(c2)C2(CC2)C(=O)N3C2CCC(=O)NC2=O)CC1 ; CC(C)(C)OC(=O)N1CCC2(CC1)OC2c1ccc2c(c1)C1(CC1)C(=O)N2C1CCC(=O)NC1=O]

CsF (0.65 g), Int. 3C46 (0.50 g) and tert-butyl 4-((4,4,5,5-tetramethyl-1,3,2-dioxaborolan-2-yl)-methylene)piperidine-1-carboxylate (0.56 g), and PdDPPFCl₂-DCM (0.12 g) were stirred 16 h in DMF (5 mL) at 100° C. under an inert atmosphere. The OL (water/EtOAc) was dried, concentrated, and purified by FC (PE/EtOAc 1:4 to 1:9) to give Int. 3U113 tert-butyl 4-((1′-(2,6-dioxopiperidin-3-yl)-2′-oxospiro[cyclopropane-1,3′-indolin]-5′-yl)methylene)piperidine-1-carboxylate (0.18 g). m-CPBA (0.12 g) was added to a solution of Int. 3U113 (0.30 g) in DCM (20 mL) at 0° C. and stirred for 2 h at RT. The OL (aq. NaHCO₃/DCM) was dried, concentrated and purified by FC (hexane/EtOAc 2:3) to give Int. 3U112 tert-butyl 2-(1′-(2,6-dioxopiperidin-3-yl)-2′-oxospiro[cyclopropane-1,3′-indolin]-5′-yl)-1-oxa-6-azaspiro[2.5]octane-6-carboxylate (0.20 g).

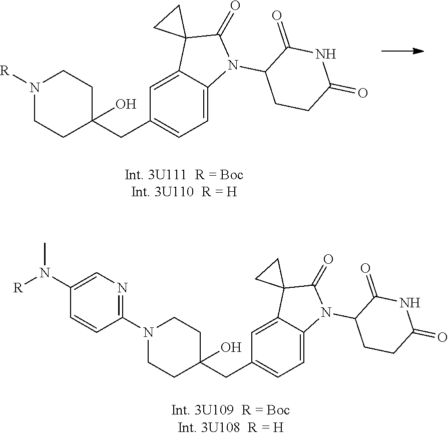

Int. 3U112 (0.20 g) and Pd/C 10% (50 mg) were hydrogenated 16 h in MeOH (10 mL). The mixture was filtered and concentrated to give Int. 3U111 tert-butyl 4-((1′-(2,6-dioxopiperidin-3-yl)-2′-oxospiro[cyclopropane-1,3′-indolin]-5′-yl)methyl)-4-hydroxypiperidine-1-carboxylate (0.12 g). Int. 3U111 (0.10 g) was stirred 2 h in DCM/TFA (25:1, 10.4 mL) at 0° C. to RT. The mixture was concentrated and washed with Et₂O to give Int. 3U110 3-(5′-((4-hydroxypiperidin-4-yl)methyl)-2′-oxospiro[cyclopropane-1,3′-indolin]-1′-yl)piperidine-2,6-dione (0.12 g, TFA salt). Ints. 1Y4/3U110 (40 mg/80 mg), Pd-PEPPSI-IHept-C1 (14 mg), and Cs₂CO₃ (0.18 g) were stirred 1 h dioxane (1 mL) at 120° C. under an inert atmosphere. The mixture was concentrated and purified by FC (DCM/MeOH 19:1) to give Int. 3U109 tert-butyl (6-(4-((1′-(2,6-dioxo-piperidin-3-yl)-2′-oxospiro[cyclopropane-1,3′-indolin]-5′-yl)methyl)-4-hydroxypiperidin-1-yl)pyridin-3-yl)(methyl)carbamate (80 mg). Int. 3U109 (70 mg) was stirred 2 h in DCM/TFA (10:1, 3.3 mL) at 0° C. to RT. The mixture was concentrated and washed with Et₂O to give Int. 3U108 3-(5′-((4-hydroxy-1-(5-(methyl-amino)pyridin-2-yl)piperidin-4-yl)methyl)-2′-oxospiro[cyclopropane-1,3′-indolin]-1′-yl)piperidine-2,6-dione (80 mg, TFA salt).

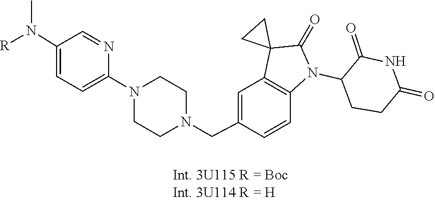

Ints. 3U94/3C44 (3.7 g/2.9 g) were stirred 24 h in DMSO/DIPEA (7:1, 40 mL). STAB (8.2 g) was added and stirring continued 16 h at 0° C. to RT. The OL (water/DCM/MeOH) was dried, concentrated, and purified by FC (DCM/MeOH 19:1 to 10:1) to give Int. 3U115 tert-butyl (6-(4-((1′-(2,6-dioxopiperidin-3-yl)-2′-oxospiro[cyclopropane-1,3′-indolin]-5′-yl)methyl)piperazin-1-yl)pyridin-3-yl)(methyl)-carbamate (0.60 g). Int. 3U115 (0.30 g) was stirred 2 h in DCM/TFA (3:2, 5 mL) at 0° C. to RT. The mixture was concentrated and washed with Et₂O to give Int. 3U114 3-(5′-((4-(5-(methylamino)-pyridin-2-yl)piperazin-1-yl)methyl)-2′-oxospiro[cyclopropane-1,3′-indolin]-1′-yl)piperidine-2,6-dione (1.8 g, TFA salt).

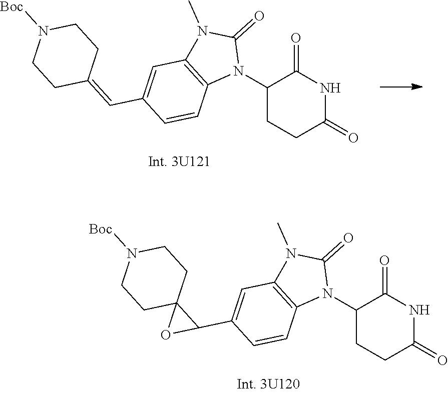 [SMILES: Cn1c(=O)n(C2CCC(=O)NC2=O)c2ccc(C=C3CCN(C(=O)OC(C)(C)C)CC3)cc21 ; Cn1c(=O)n(C2CCC(=O)NC2=O)c2ccc(C3OC34CCN(C(=O)OC(C)(C)C)CC4)cc21]

Int. 3U121 tert-butyl 4-((1-(2,6-dioxopiperidin-3-yl)-3-methyl-2-oxo-2,3-dihydro-1H-benzo[d]-imidazol-5-yl)methylene)piperidine-1-carboxylate (2.8 g) was prepared similarly to Int. 3U113 from of Int. 3B3 (5 g) and tert-butyl 4-((4,4,5,5-tetramethyl-1,3,2-dioxaborolan-2-yl)methylene)piperidine-1-carboxylate (5.7 g) and purified by FC (PE/EtOAc 1:1 to 2:3). m-CPBA (0.31 g) and Int. 3U121 (0.60 g) were stirred 1 h in DCM (10 mL) at 0° C. to RT. The OL (aq. NaHCO₃/DCM) was dried and concentrated. The residue was combined with a batch prepared similarly from Int. 3U121 (0.20 g) and purified by FC (PE/EtOAc 1:0 to 0:1) to give Int. 3U120 tert-butyl 2-(1-(2,6-dioxopiperidin-3-yl)-3-methyl-2-oxo-2,3-dihydro-1H-benzo[d]imidazol-5-yl)-1-oxa-6-azaspiro[2.5]octane-6-carboxylate (0.50 g).

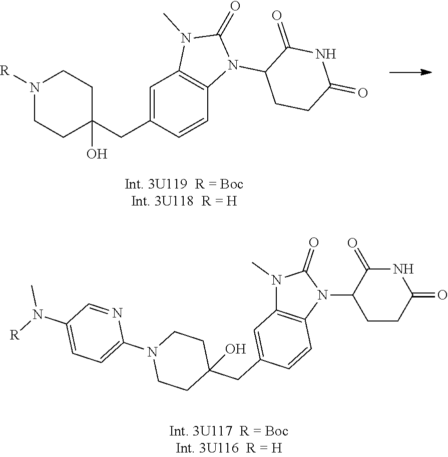

Int. 3U120 (0.50 g) and Pd/C 10% wet (0.12 g) were hydrogenated 20 h at 50 psi in EtOAc (10 mL). The mixture was filtered, concentrated, and purified by FC (PE/EtOAc 1:0 to 0:1) to give Int. 3U119 tert-butyl 4-((1-(2,6-dioxopiperidin-3-yl)-3-methyl-2-oxo-2,3-dihydro-1H-benzo[d]imidazol-5-yl)-methyl)-4-hydroxy-piperidine-1-carboxylate (0.26 g). Int. 3U119 (0.25 g) was stirred 5 h in DCM/4M HCl in dioxane (5:1, 6 mL) at 0° C. to RT. The residue after concentration washed with Et₂O to give Int. 3U118 3-(5-((4-hydroxy-piperidin-4-yl)methyl)-3-methyl-2-oxo-2,3-dihydro-1H-benzo[d]imidazol-1-yl)piperidine-2,6-dione (0.18 g, HCl salt). Ints. 1Y4/3U118 (0.20 g/0.34 g), Cs₂CO₃ (0.68 g), and Pd-PEPPSI-IHept-C1 (68 mg) were stirred 1 h in dioxane (3 mL) at 110° C. under an inert atmosphere of argon. The mixture was filtered. The OL (water/EtOAc) was dried, concentrated, and purified by FC (DCM/MeOH 1:0 to 9:1) to give Int. 3U117 tert-butyl (6-(4-((1-(2,6-dioxopiperidin-3-yl)-3-methyl-2-oxo-2,3-dihydro-1H-benzo[d]imidazol-5-yl)methyl)-4-hydroxy-piperidin-1-yl)pyridin-3-yl)(methyl)-carbamate (0.27 g). Int. 3U117 (0.30 g) was stirred 5 h in DCM/4M HCl in dioxane (5:2, 7 mL) at 0° C. to RT. The residue after concentration was washed with Et₂O to give Int. 3U116 3-(5-((4-hydroxy-1-(5-(methylamino)pyridin-2-yl)piperidin-4-yl)methyl)-3-methyl-2-oxo-2,3-dihydro-1H-benzo[d]-imidazol-1-yl)piperidine-2,6-dione (0.24 g, HCl salt).

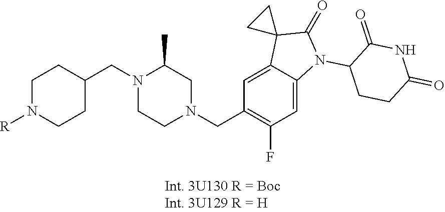

Ints. 3E74/3C6 (1.5 g/4.0 g) were stirred 24 h in DMSO/DIPEA (5:2, 28.3 mL). STAB (7.1 g) was added and stirring continued 16 h at 0° C. to RT. The OL (water/EtOAc) was dried, concentrated, and purified by FC (DCM/MeOH 1:0 to 19:1) to give Int. 3U130 tert-butyl 4-(((2S)-4-((1′-(2,6-dioxopiperidin-3-yl)-6′-fluoro-2′-oxospiro[cyclopropane-1,3′-indolin]-5′-yl)methyl)-2-methylpiperazin-1-yl)methyl)piperidine-1-carboxylate (0.80 g). Int. 3U130 (1.0 g) was stirred 1 h in DCM/4M HCl in dioxane (3:1, 13 mL) at 0° C. to RT. The residue after concentration was washed with Et₂O to give Int. 3U129 3-(6′-fluoro-5′-(((S)-3-methyl-4-(piperidin-4-ylmethyl)piperazin-1-yl)methyl)-2′-oxospiro[cyclopropane-1,3′-indolin]-1′-yl)piperidine-2,6-dione (0.80 g, HCl salt).

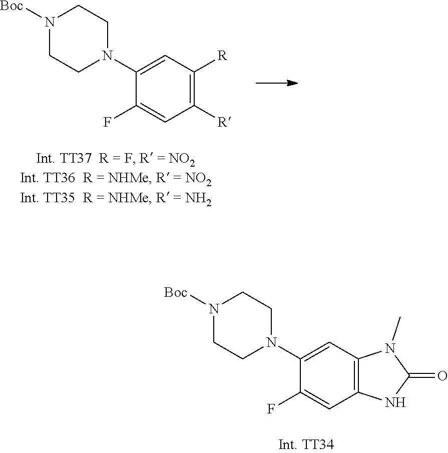

1,2,4-Trifluoro-5-nitrobenzene (19 g) and K₂CO₃ (15 g) was stirred in DMF (100 mL) for 15 min. tert-Butyl piperazine-1-carboxylate (20 g) in THF (25 mL) was added at 0° C. and stirred for 4 h at RT and concentrated. The OL (water/EtOAc) was dried, filtered, concentrated, and purified by FC (PE/EtOAc 9:1) to give Int. 2T30 tert-butyl 4-(2,5-difluoro-4-nitrophenyl)piperazine-1-carboxylate (30 g). 2M Methylamine in THF (219 mL) was added to a mixture of Int. 2T30 (30 g) in THF (300 mL) and stirred for 4 h. The OL (water/EtOAc) was dried, filtered, concentrated and purified by FC (PE/EtOAc 9:1) to give Int. 2T31 tert-butyl 4-(2-fluoro-5-(methylamino)-4-nitrophenyl)piperazine-1-carboxylate (26 g). Int. 2T31 (10 g) was hydrogenated for 16 h at 100° C. using 10% Pd/C (5 g) in MeOH (150 mL), filtered and concentrated to give Int. 2T32 tert-butyl 4-(4-amino-2-fluoro-5-(methylamino)phenyl)piperazine-1-carboxylate (7.0 g). Int. 2T32 (8 g) and CDI (6 g) were stirred in ACN (50 mL) for 12 h at 80° C. The OL (water/EtOAc) was dried, filtered, concentrated and purified by FC (PE/EtOAc 3:17) to give Int. 2T34 tert-butyl 4-(6-fluoro-3-methyl-2-oxo-2,3-dihydro-1H-benzo[d]imidazol-5-yl)piperazine-1-carboxylate (5.0 g).

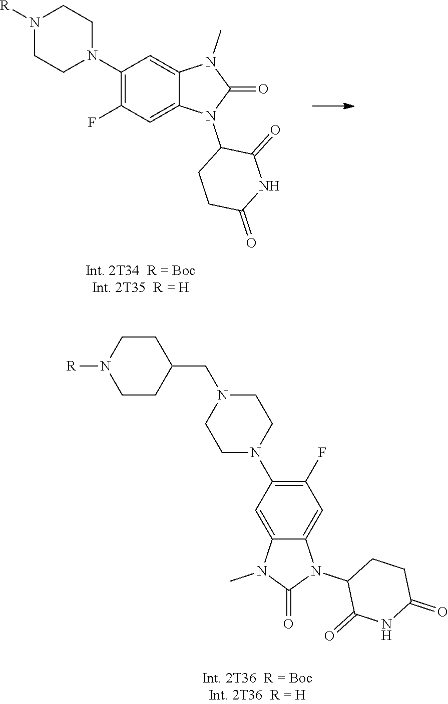

Int. 2T34 tert-butyl 4-(1-(2,6-dioxopiperidin-3-yl)-6-fluoro-3-methyl-2-oxo-2,3-dihydro-1H-benzo[d]imidazol-5-yl)piperazine-1-carboxylate (2.3 g) was prepared similarly to Int. 3U36 from Int. 2T33 (5.0 g) and 3-bromopiperidine-2,6-dione (14 g). Int. 2T35 3-(6-fluoro-3-methyl-2-oxo-5-(piperazin-1-yl)-2,3-dihydro-1H-benzo[d]imidazol-1-yl)piperidine-2,6-dione (1.6 g, HCl salt) was prepared similarly to Int. 2T39 from Int. 2T34 (2.0 g). Int. 2T35 (2. 0 g) and tert-butyl 4-formylpiperidine-1-carboxylate (0.90 g) were stirred in DCE/DIPEA (7:2, 19.4 mL) for 16 h under an inert atmosphere. STAB (3.6 g) was added at 0° C. and stirring continued for 4 h. The OL (water/DCM) was dried, filtered, concentrated and purified by FC (PE/EtOAc 3:2) to give Int. 2T36 tert-butyl 4-((4-(1-(2,6-dioxopiperidin-3-yl)-6-fluoro-3-methyl-2-oxo-2,3-dihydro-1H-benzo[d]imidazol-5-yl)piperazin-1-yl)methyl)piperidine-1-carboxylate (1.3 g). Int. 2T37 3-(6-fluoro-3-methyl-2-oxo-5-(4-(piperidin-4-ylmethyl)piperazin-1-yl)-2,3-dihydro-1H-benzo[d]imidazol-1-yl)piperidine-2,6-dione (0.80 g, HCl salt) was prepared similarly to Int. 2T39 from Int. 2T36 (1.2 g).

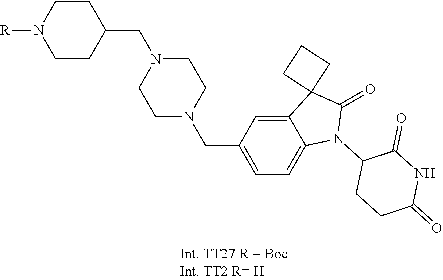

Int. 2T38 tert-butyl 4-((4-((1′-(2,6-dioxopiperidin-3-yl)-2′-oxospiro[cyclobutane-1,3′-indolin]-5′-yl)-methyl)piperazin-1-yl)methyl)piperidine-1-carboxylate (0.12 g) was prepared similarly to Int. 3U31 from Int. 3U32 (0.20 g) and tert-butyl 4-(piperazin-1-ylmethyl)piperidine-1-carboxylate (0.36 g). Int. 2T38 (0.12 g) was mixed in DCM/4M HCl in dioxane (1:2, 15 mL) at 0° C. and stirred for 6 h at RT. The mixture was concentrated and washed with Et₂O to give Int. 2T39 3-(2′-oxo-5′-((4-(piperidin-4-ylmethyl)-piperazin-1-yl)methyl)spiro[cyclobutane-1,3′-indolin]-1′-yl)piperidine-2,6-dione (0.10 g, HCl salt).

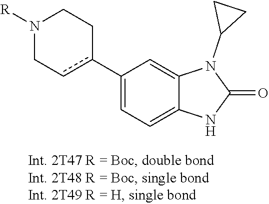

6-Bromo-1-cyclopropyl-1,3-dihydro-2H-benzo[d]imidazol-2-one (7 g), tert-butyl 4-(4,4,5,5-tetramethyl-1,3,2-dioxaborolan-2-yl)-3,6-dihydropyridine-1 (2H)-carboxylate (9 g), K₂CO₃ (15 g) and PdDPPFCl₂-DCM (2 g) were stirred in dioxane/H₂O (4:1, 200 mL) for 12 h at 80° C. under an inert atmosphere. The mixture was filtered and concentrated to give Int. 2T47 tert-butyl 4-(3-cyclopropyl-2-oxoindolin-5-yl)-3,6-dihydropyridine-1 (2H)-carboxylate (1.2 g). Int. 2T47 (1.2 g) was hydrogenated for 12 h using Pd/C (0.5 g) in MeOH (50 mL), filtered and concentrated to give Int. 2T48 tert-butyl 4-(3-cyclopropyl-2-oxo-2,3-dihydro-1H-benzo[d]imidazol-5-yl)-piperidine-1-carboxylate (1.2 g). Int. 2T48 (1.2 g) was stirred ON in MeOH/2.8M HCl in dioxane (5:1, 60 mL) and concentrated to give Int. 2T49 1-cyclopropyl-6-(piperidin-4-yl)-1,3-dihydro-2H-benzo[d]imidazol-2-one (0.5 g).

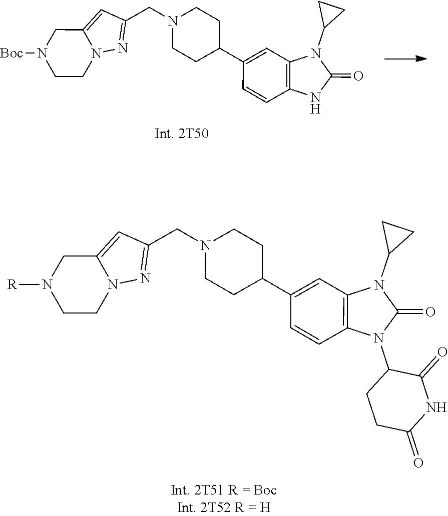

Ints. 2Q11/2T49 (0.4 g/0.5 g) and STAB (0.9 g) were stirred in DCE/DIPEA (74:1, 50.7 mL) for 48 h under an inert atmosphere. The OL (water/DCE) was concentrated to give Int. 2T50 tert-butyl 2-((4-(3-cyclopropyl-2-oxo-2,3-dihydro-1H-benzo[d]imidazol-5-yl)piperidin-1-yl)methyl)-6,7-dihydropyrazolo[1,5-a]pyrazine-5 (4H)-carboxylate (0.4 g). Int. 2T51 tert-butyl 2-((4-(3-cyclopropyl-1-(2,6-dioxopiperidin-3-yl)-2-oxo-2,3-dihydro-1H-benzo[d]imidazol-5-yl)piperidin-1-yl)methyl)-6,7-dihydropyrazolo[1,5-a]pyrazine-5 (4H)-carboxylate (0.21 g) was prepared similarly to Int. YY45 from Int. 2T50 (0.4 g) and 3-bromopiperidine-2,6-dione (0.5 g). Int. 2T51 (0.21 g) was stirred ON in MeOH/2.8M HCl in dioxane (4:1, 25 mL), concentrated and crystallized from ? to give Int. 2T52 3-(3-cyclopropyl-2-oxo-5-(1-((4,5,6,7-tetrahydropyrazolo[1,5-a]pyrazin-2-yl)methyl)piperidin-4-yl)-2,3-dihydro-1H-benzo[d]imidazol-1-yl)piperidine-2,6-dione (0.12 g, HCl salt).

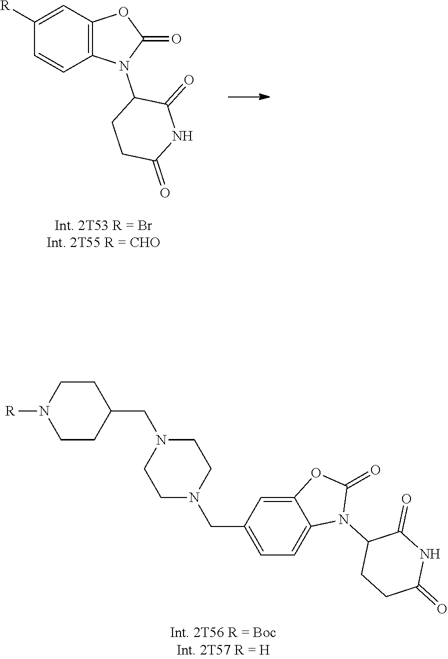

Int. 2T53 3-(6-bromo-2-oxobenzo[d]oxazol-3 (2H)-yl)piperidine-2,6-dione (10 g) was prepared similarly to Int. 3U36 from 6-bromobenzo[d]oxazol-2 (3H)-one (15 g) and 3-bromopiperidine-2,6-dione (135 g). tert-Butyl (E)-3-(3-(2,6-dioxopiperidin-3-yl)-2-oxo-2,3-dihydrobenzo[d]oxazol-6-yl)acrylate (7 g) was prepared similarly to Int. 3U33 from Int. 2T53 (10 g) and tert-butyl acrylate (22 mL). Int. 2T55 3-(2,6-dioxopiperidin-3-yl)-2-oxo-2,3-dihydrobenzo[d]oxazole-6-carbaldehyde (2.5 g) was prepared similarly to Int. 3U32 from Int. 2T54 (7 g). Int. 2T54 tert-butyl 4-((4-((3-(2,6-dioxo-piperidin-3-yl)-2-oxo-2,3-dihydrobenzo[d]oxazol-6-yl)methyl)-piperazin-1-yl)methyl)piperidine-1-carboxylate (2 g) was prepared similarly to Int. 3U31 from Int. 2T55 (2.5 g) and tert-butyl 4-(piperazin-1-ylmethyl)piperidine-1-carboxylate (5.2 g). Int. 2T56 (0.60 g) was stirred in DCM/TFA (5:1, 12 mL) for 16 h. The mixture was concentrated, triturated in Et₂O to give Int. 2T57 3-(2-oxo-6-((4-(piperidin-4-ylmethyl)piperazin-1-yl)methyl)benzo[d]oxazol-3 (2H)-yl)piperidine-2,6-dione (0.55 g, TFA salt).

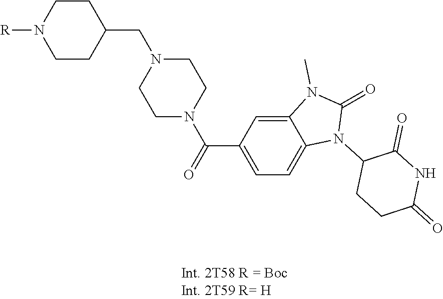

Int. 3B3 (2 g), tert-butyl 4-(piperazin-1-ylmethyl)piperidine-1-carboxylate (2.5 g) and PdDPPFCl₂-DCM (0.48 g) in dioxane/Et₃N (12:1, 32.5 mL) and stirred for 16 h at 100° C. under 200 psi of CO. The mixture was filtered, concentrated and purified by FC (DCM/MeOH 9:1) to give Int. 2T58 tert-butyl 4-((4-(1-(2,6-dioxopiperidin-3-yl)-3-methyl-2-oxo-2,3-dihydro-1H-benzo[d]imidazole-5-carbonyl)-piperazin-1-yl)methyl)piperidine-1-carboxylate (0.90 g). Int. 2T59 3-(3-methyl-2-oxo-5-(4-(piperidin-4-ylmethyl)piperazine-1-carbonyl)-2,3-dihydro-1H-benzo[d]imidazol-1-yl)piperidine-2,6-dione (0.35, HCl salt) was prepared similarly to Int. 2T39 from Int.2T58 (0.50 g).

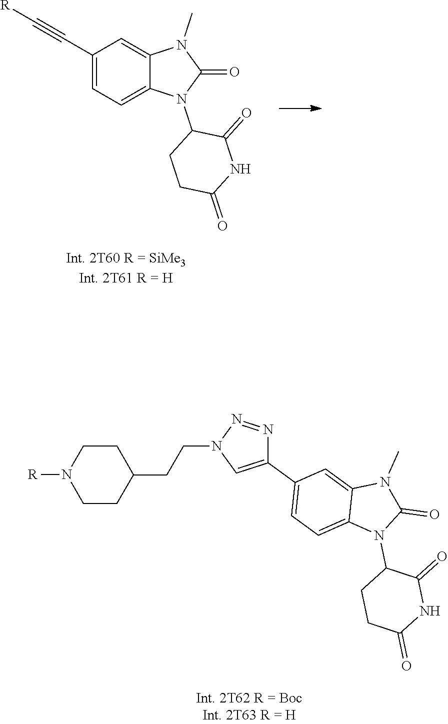

Int. 3B3 (2 g), ethynyltrimethylsilane (1.6 mL), Pd(PPh₃)₂Cl₂ (0.41 g), CuI (0.34 g) and Et₃N (2.1 mL) were stirred in DMF (15 mL) for 2 h at 80° C. under an inert atmosphere under MW conditions. The OL (water/EtOAc) was dried, filtered, concentrated, and purified by FC (PE/EtOAc 1:0 to 1:1) to give Int. 2T60 3-(3-methyl-2-oxo-5-((trimethylsilyl)ethynyl)-2,3-dihydro-1H-benzo[d]imidazol-1-yl)piperidine-2,6-dione (0.80 g). CsF (0.68 g) was added to a solution of Int. 2T60 (0.80 g) in DMSO (8 mL) at 0° C. and stirred for 6 h. The OL (water/EtOAc) was dried, filtered and concentrated to give Int. 2T61 3-(5-ethynyl-3-methyl-2-oxo-2,3-dihydro-1H-benzo[d]imidazol-1-yl)piperidine-2,6-dione (0.60 g). Sodium ascorbate (1.7 g) was added to a mixture of Int. 2T61 (0.80 g), tert-butyl 4-(2-azidoethyl)piperidine-1-carboxylate (0.86 g) and CuSO₄·H₂O (0.90 g) in ^{t}BuOH/H₂O (5:4, 18 mL), stirred for 4 h and filtered. The OL (water/EtOAc) was concentrated to give Int. 2T62 tert-butyl 4-(2-(4-(1-(2,6-dioxopiperidin-3-yl)-3-methyl-2-oxo-2,3-dihydro-1H-benzo[d]-imidazol-5-yl)-1H-1,2,3-triazol-1-yl)ethyl)piperidine-1-carboxylate (0.60 g). Int. 2T63 3-(3-methyl-2-oxo-5-(1-(2-(piperidin-4-yl)ethyl)-1H-1,2,3-triazol-4-yl)-2,3-dihydro-1H-benzo[d]imidazol-1-yl)piperidine-2,6-dione (0.40 g, HCl salt) was prepared similarly to Int. 2T39 from Int. 2T62 (0.60 g).

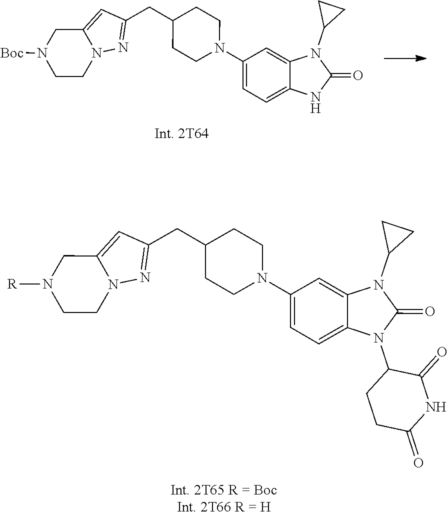

tert-Butyl 2-(piperidin-4-ylmethyl)-6,7-dihydropyrazolo[1,5-a]pyrazine-5 (4H)-carboxylate (0.6 g), 6-bromo-1-cyclopropyl-1,3-dihydro-2H-benzo[d]imidazol-2-one (0.5 g), RuPhos (90 mg) and 1.1M LiHMDS in THF (6 mL) were stirred in toluene (50 mL) at 110° C. under an inert atmosphere. RuPhos Pd-G2 (10 mg) was added and stirring continued for 14 h at 100° C. The mixture was filtered, concentrated and purified by FC (CHCl₃/ACN 1:0 to 3:7) to give Int. 2T64 tert-butyl 2-((1-(3-cyclopropyl-2-oxo-2,3-dihydro-1H-benzo[d]imidazol-5-yl)-piperidin-4-yl)methyl)-6,7-dihydropyrazolo[1,5-a]pyrazine-5 (4H)-carboxylate (0.1 g). Int. 2T65 tert-butyl 2-((1-(3-cyclopropyl-1-(2,6-dioxopiperidin-3-yl)-2-oxo-2,3-dihydro-1H-benzo[d]imidazol-5-yl)piperidin-4-yl)-methyl)-6,7-dihydropyrazolo[1,5-a]pyrazine-5 (4H)-carboxylate (0.15 g) was prepared similarly to Int. 3U36 from Int. 2T64 (0.1 g) and 3-bromopiperidine-2,6-dione (0.2 g). Int. 2T65 (0.15 g) was stirred in DCM/TFA (165:1, 20.1 mL) for 14 h, concentrated and HPLC-purified to give Int. 2T66 3-(3-cyclopropyl-2-oxo-5-(4-((4,5,6,7-tetrahydropyrazolo[1,5-a]pyrazin-2-yl)methyl)piperidin-1-yl)-2,3-dihydro-1H-benzo[d]imidazol-1-yl)piperidine-2,6-dione (25 mg, TFA salt).

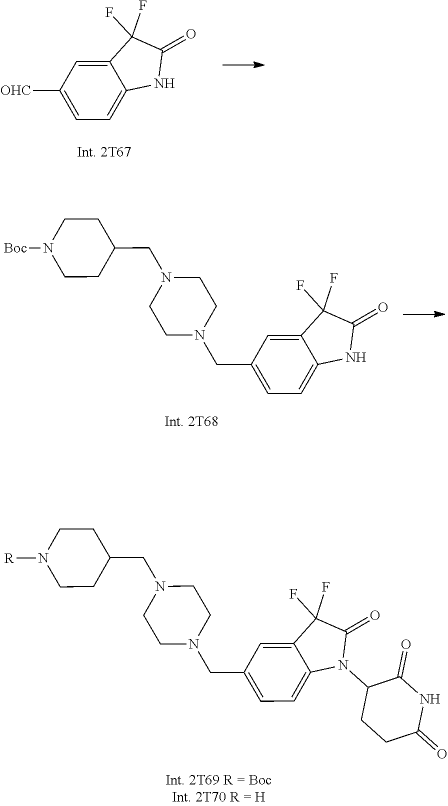

5-bromo-3,3-difluoroindolin-2-one (6 g), Et₃SiH (12 mL) and PdDPPFCl₂-DCM (2.0 g) in DMF/Et₃N (16:1, 85 mL) and stirred for 16 h at 130° C. under 440 psi of CO. The OL (water/EtOAc) was dried, filtered, concentrated and purified by FC (CHCl₃/ACN 1:0 to 6:1) to give Int. 2T67 3,3-difluoro-2-oxoindoline-5-carbaldehyde (2.2 g). Int. 2T68 tert-butyl 4-((4-((3,3-difluoro-2-oxoindolin-5-yl)-methyl)piperazin-1-yl)methyl)piperidine-1-carboxylate (1.1 g) was prepared similarly to Int. 3U37 from Int. 2T67 (1 g) and tert-butyl 4-(piperazin-1-ylmethyl)piperidine-1-carboxylate (1.5 g). Int. 2T69 tert-butyl 4-((4-((1-(2,6-dioxo-piperidin-3-yl)-3,3-difluoro-2-oxoindolin-5-yl)methyl)piperazin-1-yl)-methyl)piperidine-1-carboxylate (0.2 g) was prepared similarly to Int. 3U36 from Int. 2T68 (0.7 g) and 3-bromopiperidine-2,6-dione (1 g). Int. 2T69 (0.2 g) was stirred in DCM/4M HCl in dioxane (2:1, 60 mL) for 14 h and concentrated to give Int. 2T70 3-(3,3-difluoro-2-oxo-5-((4-(piperidin-4-ylmethyl)-piperazin-1-yl)methyl)indolin-1-yl)piperidine-2,6-dione (0.28 g, HCl salt).

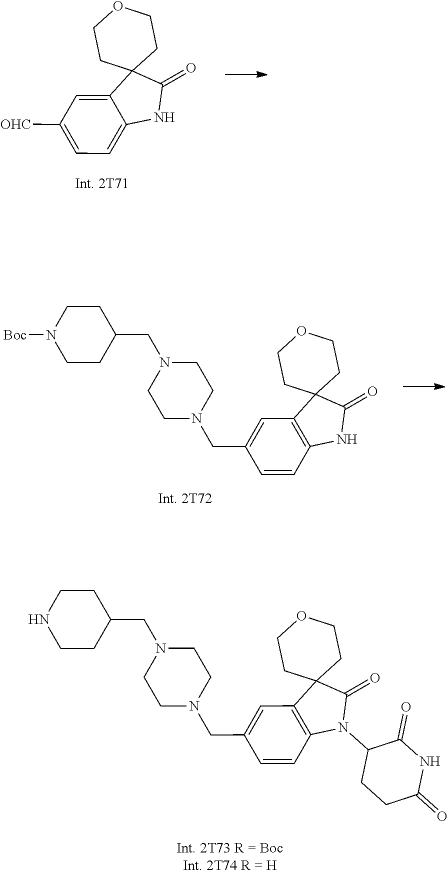 [SMILES: O=Cc1ccc2c(c1)C1(CCOCC1)C(=O)N2 ; CC(C)(C)OC(=O)N1CCC(CN2CCN(Cc3ccc4c(c3)C3(CCOCC3)C(=O)N4)CC2)CC1 ; O=C1CCC(N2C(=O)C3(CCOCC3)c3cc(CN4CCN(CC5CCNCC5)CC4)ccc32)C(=O)N1]

5-Bromo-2′,3′,5′,6′-tetrahydrospiro[indoline-3,4′-pyran]-2-one (5 g), Et₃SiH (5 mL) and PdDPPFCl₂-DCM (1 g) in DMA/Et₃N (10:1, 55 mL) and stirred for 12 h at 130° C. under 294 psi of CO. The OL (water/EtOAc) was dried, filtered, concentrated and HPLC-purified to give Int. 2T71 2-oxo-2′,3′,5′,6′-tetrahydrospiro[indoline-3,4′-pyran]-5-carbaldehyde (2.8 g). Int. 2T71 (2.8 g), tert-butyl 4-(piperazin-1-ylmethyl)piperidine-1-carboxylate (3.4 g) and STAB (10 g) were stirred in DCE (50 mL) for 48 h. The OL (water/DCE) was concentrated to give Int. 2T72 tert-butyl 4-((4-((2-oxo-2′,3′,5′,6′-tetrahydro-spiro[indoline-3,4′-pyran]-5-yl)-methyl)piperazin-1-yl)methyl)piperidine-1-carboxylate (1.6 g). Int. 2T73 tert-butyl 4-((4-((1-(2,6-dioxo-piperidin-3-yl)-2-oxo-2′,3′,5′,6′-tetrahydrospiro[indoline-3,4′-pyran]-5-yl)methyl)piperazin-1-yl)methyl)-piperidine-1-carboxylate (0.7 g) was prepared similarly to Int. 3U36 from Int. 2T72 (1.6 g) and 3-bromo-piperidine-2,6-dione (2.2 g). Int. 2T73 (0.45 g) was stirred in MeOH/4M HCl in dioxane (4:1, 25 mL) ON, concentrated and precipitated from EtOH to give Int. 2T74 3-(2-oxo-5-((4-(piperidin-4-ylmethyl)piperazin-1-yl)methyl)-2′,3′,5′,6′-tetrahydrospiro[indoline-3,4′-pyran]-1-yl)piperidine-2,6-dione (0.2, HCl salt).

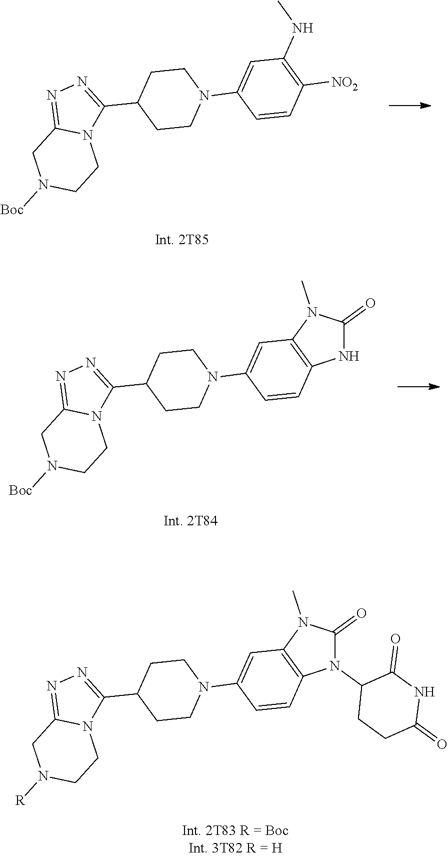

5-Fluoro-N-methyl-2-nitroaniline (0.6 g), tert-butyl 3-(piperidin-4-yl)-5,6-dihydro-[1,2,4]triazolo[4,3-a]pyrazine-7 (8H)-carboxylate (1 g), and K₂CO₃ (0.5 g) were stirred 8 h in DMSO (50 mL) at 80° C. The mixture was filtered and concentrated to give Int. 2T85 tert-butyl 3-(1-(3-(methylamino)-4-nitro-phenyl)piperidin-4-yl)-5,6-dihydro-[1,2,4]triazolo[4,3-a]pyrazine-7 (8H)-carboxylate (0.6 g), which was mixed with CDI (2 g) and Pd/C and hydrogenated for 12 h in EtOAc (40 mL). The mixture was filtered and concentrated to give Int. 2T84 tert-butyl 3-(1-(3-methyl-2-oxo-2,3-dihydro-1H-benzo[d]imidazol-5-yl)piperidin-4-yl)-5,6-dihydro-[1,2,4]triazolo[4,3-a]pyrazine-7 (8H)-carboxylate (0.2 g). Int. 2T83 tert-butyl 3-(1-(1-(2,6-dioxopiperidin-3-yl)-3-methyl-2-oxo-2,3-dihydro-1H-benzo[d]imidazol-5-yl)-piperidin-4-yl)-5,6-dihydro-[1,2,4]triazolo[4,3-a]-pyrazine-7 (8H)-carboxylate (0.15 g) was prepared similarly to Int. 3U36 from Int. 2T84 (0.2 g) and 3-bromo-piperidine-2,6-dione (0.4 g). Int. 2T83 (0.15 g) was stirred in DCM/TFA (175:1, 20.1 mL) for 14 h. The mixture was concentrated and HPLC-purified to give Int. 2T82 3-(3-methyl-2-oxo-5-(4-(5,6,7,8-tetrahydro-[1,2,4]-triazolo[4,3-a]pyrazin-3-yl)piperidin-1-yl)-2,3-dihydro-1H-benzo[d]imidazol-1-yl)piperidine-2,6-dione (25 mg).

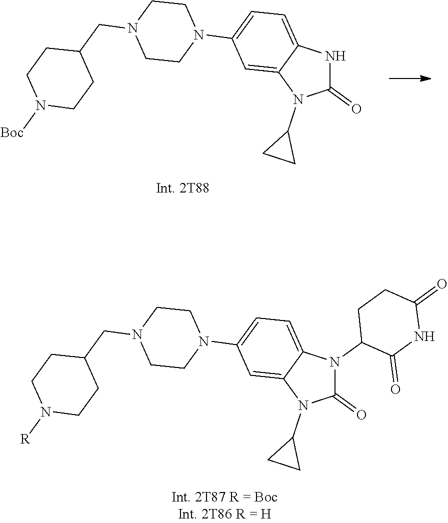

Int. 2T88 tert-butyl 4-((4-(3-cyclopropyl-2-oxo-2,3-dihydro-1H-benzo[d]imidazol-5-yl)piperazin-1-yl)methyl)-piperidine-1-carboxylate (2 g) was prepared similarly to Int. 3U9 from 6-bromo-1-cyclo-propyl-1,3-dihydro-2H-benzo[d]imidazol-2-one (5.3 g) and tert-butyl 4-(piperazin-1-ylmethyl)-piperidine-1-carboxylate (5 g). Int. 2T87 tert-butyl 4-((4-(3-cyclopropyl-1-(2,6-dioxopiperidin-3-yl)-2-oxo-2,3-dihydro-1H-benzo[d]imidazol-5-yl)piperazin-1-yl)methyl)piperidine-1-carboxylate (2.1 g) was prepared similarly to Int. 3U36 from Int. 2T88 (2 g) and 3-bromopiperidine-2,6-dione (5.9 g). Int. 2T87 (2.1 g) was stirred in ACN/TFA (3:1, 20 mL) for 16 h. The mixture was concentrated to give Int. 2T86 3-(3-cyclopropyl-2-oxo-5-(4-(piperidin-4-ylmethyl)piperazin-1-yl)-2,3-dihydro-1H-benzo[d]-imidazol-1-yl)piperidine-2,6-dione (1.4 g, TFA salt).

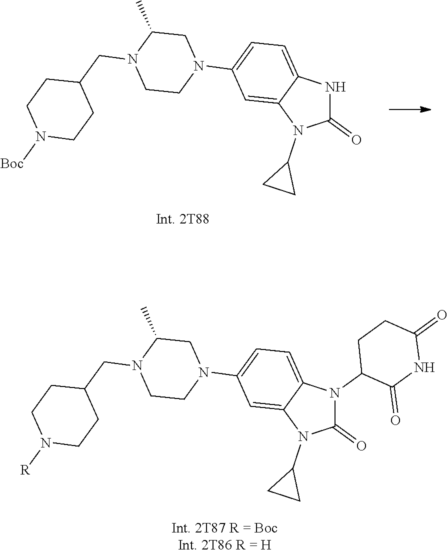

Int. 2T91 tert-butyl (R)-4-((2-methyl-4-(2′-oxospiro[cyclopropane-1,3′-indolin]-5′-yl)piperazin-1-yl)methyl)-piperidine-1-carboxylate (2.5 g) was prepared similarly to Int. 3U9 from Int. 2F12 (4.5 g) and 5′-bromospiro-[cyclopropane-1,3′-indolin]-2′-one (3 g). Int. 2T90 tert-butyl 4-(((2R)-4-(1′-(2,6-dioxopiperidin-3-yl)-2′-oxospiro[cyclopropane-1,3′-indolin]-5′-yl)-2-methylpiperazin-1-yl)methyl)-piperidine-1-carboxylate (0.50 g) was prepared similarly to Int. YY45 from Int. 2T91 (1 g) and 3-bromopiperidine-2,6-dione (3.0 g). Int. 2T90 (0.55 g) was stirred in DCM/TFA (15:2, 17 mL) for 12 h. The mixture was concentrated and triturated in Et₂O/EtOAc (1:1) to give Int. 2T89 3-(5′-((R)-3-methyl-4-(piperidin-4-ylmethyl)piperazin-1-yl)-2′-oxospiro-[cyclopropane-1,3′-indolin]-1′-yl)-piperidine-2,6-dione (0.65 g, TFA salt).

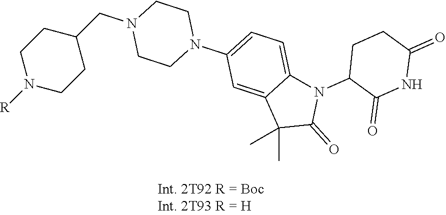

Int. 2T92 tert-butyl 4-((4-(1-(2,6-dioxopiperidin-3-yl)-3,3-dimethyl-2-oxoindolin-5-yl)piperazin-1-yl)methyl)-piperidine-1-carboxylate (0.70 g) was prepared similarly to Int. 3U9 from Int. 3E295 (1.0 g) and tert-butyl 4-(piperazin-1-ylmethyl)piperidine-1-carboxylate (1.2 g) by stirring for 2 h at 120° C. Int. 2T92 (0.70 g) was stirred 16 h in DCM/4M HCl in dioxane (5:1, 24 mL) at 10° C. to RT. The mixture was concentrated and triturated in ACN and Et₂O to give Int. 2T93 3-(3,3-dimethyl-2-oxo-5-(4-(piperidin-4-ylmethyl)piperazin-1-yl)indolin-1-yl)piperidine-2,6-dione (0.6 g, HCl salt).

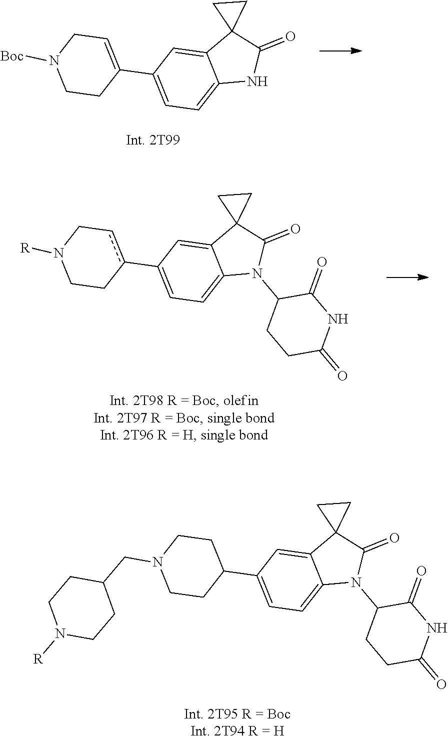

Int. 2T99 tert-butyl 4-(2′-oxospiro[cyclopropane-1,3′-indolin]-5′-yl)-3,6-dihydropyridine-1 (2H)-carboxylate (0.75 g) was prepared similarly to Int. 3U37 from Int. 5′-bromospiro[cyclopropane-1,3′-indolin]-2′-one (1.0 g), tert-butyl 4-(4,4,5,5-tetramethyl-1,3,2-dioxaborolan-2-yl)-3,6-dihydro-pyridine-1 (2H)-carboxylate (1.7 g) and NaHCO₃ (1.1 g). Int. 2T98 tert-butyl 4-(1′-(2,6-dioxopiperidin-3-yl)-2′-oxospiro[cyclopropane-1,3′-indolin]-5′-yl)-3,6-dihydropyridine-1 (2H)-carboxylate (2.3 g) was prepared similarly to Int. 3U36 from Int. TT100 (2.3 g) and 3-bromopiperidine-2,6-dione (6.5 g). Int. 2T98 (2.3 g) was hydrogenated for 16 h using 10% wet Pd/C (1.0 g) in THF/EtOAc (1:1, 20 mL), filtered, concentrated and triturated in Et₂O to give Int. 2T97 tert-butyl 4-(1′-(2,6-dioxopiperidin-3-yl)-2′-oxospiro[cyclopropane-1,3′-indolin]-5′-yl)piperidine-1-carboxylate (2.0 g). Int. 2T97 (2.0 g) was mixed in DCM/4M HCl in dioxane (15:1, 32 mL) at 0° C. and stirred for 16 h at RT. The mixture was concentrated and triturated in ACN and Et₂O to give Int. 2T96 3-(2′-oxo-5′-(piperidin-4-yl)spiro-[cyclopropane-1,3′-indolin]-1′-yl)piperidine-2,6-dione (2.0 g, TFA salt). Int. 2T95 tert-butyl 4-((4-(1′-(2,6-dioxopiperidin-3-yl)-2′-oxospiro[cyclopropane-1,3′-indolin]-5′-yl)piperidin-1-yl)methyl)-piperidine-1-carboxylate (0.90 g) was prepared similarly to Int. 2T36 from Int. 2T96 (2.0 g) and tert-butyl 4-formyl-piperidine-1-carboxylate (0.90 g) in DMSO (30 mL). Int. 2T94 3-(2′-oxo-5′-(1-(piperidin-4-ylmethyl)piperidin-4-yl)spiro[cyclopropane-1,3′-indolin]-1′-yl)piperidine-2,6-dione (0.68 g, TFA salt) was prepared similarly to Int. 2T36 from Int. 2T95 (0.90 g).

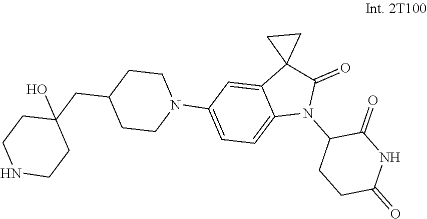 [SMILES: O=C1CCC(N2C(=O)C3(CC3)c3cc(N4CCC(CC5(O)CCNCC5)CC4)ccc32)C(=O)N1]

Int. 3U16 (0.50 g) was stirred in DCM/TFA (5:3, 8 mL) for 3 h, concentrated and washed with Et₂O to give Int. 2T100 3-(5′-(4-((4-hydroxypiperidin-4-yl)methyl)piperidin-1-yl)-2′-oxospiro[cyclopropane-1,3′-indolin]-1′-yl)-piperidine-2,6-dione (0.50 g, TFA salt).

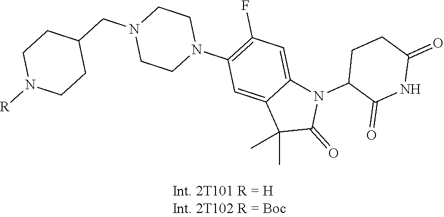

Int. 3C51 (0.50 g), tert-butyl 4-(piperazin-1-ylmethyl)piperidine-1-carboxylate (0.58 g), Cs₂CO₃ (1.3 g) and Pd-PEPPSI-IHept-C1 (66 mg) were stirred in dioxane (10 mL) for 16 h at 110° C. under an inert atmosphere and concentrated. The residue was combined with three batches prepared similarly and purified by FC (DCM/MeOH 9:1) and HPLC to give Int. 2T102 tert-butyl 4-((4-(1-(2,6-dioxopiperidin-3-yl)-6-fluoro-3,3-dimethyl-2-oxoindolin-5-yl)piperazin-1-yl)methyl)piperidine-1-carboxylate (80 mg). Int. 2T102 (80 mg) was stirred 12 h in DCM/4M HCl in dioxane (2:5, 7 mL) at 0° C. to RT. The residue after concentration was washed with Et₂O to give Int. 2T101 3-(6-fluoro-3,3-dimethyl-2-oxo-5-(4-(piperidin-4-ylmethyl)piperazin-1-yl)indolin-1-yl)piperidine-2,6-dione (60 mg, HCl salt).

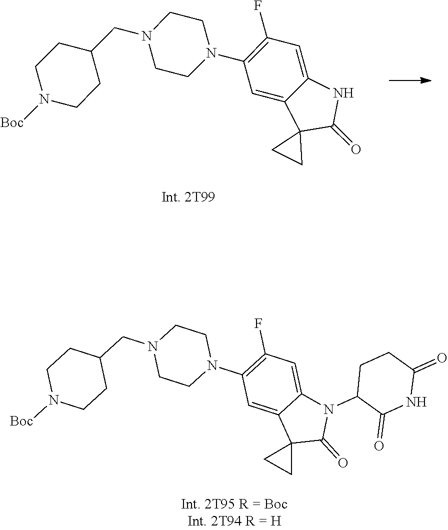 [SMILES: CC(C)(C)OC(=O)N1CCC(CN2CCN(c3cc4c(cc3F)NC(=O)C43CC3)CC2)CC1 ; CC(C)(C)OC(=O)N1CCC(CN2CCN(c3cc4c(cc3F)N(C3CCC(=O)NC3=O)C(=O)C43CC3)CC2)CC1]

Int. 2T103 tert-butyl 4-((4-(6′-fluoro-2′-oxospiro[cyclopropane-1,3′-indolin]-5′-yl)piperazin-1-yl)-methyl)piperidine-1-carboxylate (0.60 g) was prepared similarly to Int. 3U9 from Int. 3C9 (1 g) and tert-butyl 4-(piperazin-1-ylmethyl)piperidine-1-carboxylate (2.2 g). Int. 2T104 tert-butyl 4-((4-(1′-(2,6-dioxopiperidin-3-yl)-6′-fluoro-2′-oxospiro[cyclopropane-1,3′-indolin]-5′-yl)piperazin-1-yl)-methyl)piperidine-1-carboxylate (0.55 g) was prepared similarly to Int. 3U36 from Int. 2T103 (1 g) and 3-bromopiperidine-2,6-dione (2.1 g). Int. 2T104 (0.55 g) was stirred 16 h in DCM/TFA (10:1, 11 mL). The residue after concentration washed with Et₂O/PE to give Int. 2T105 3-(6′-fluoro-2′-oxo-5′-(4-(piperidin-4-ylmethyl)piperazin-1-yl)spiro[cyclopropane-1,3′-indolin]-1′-yl)piperidine-2,6-dione (0.50 g, TFA salt).

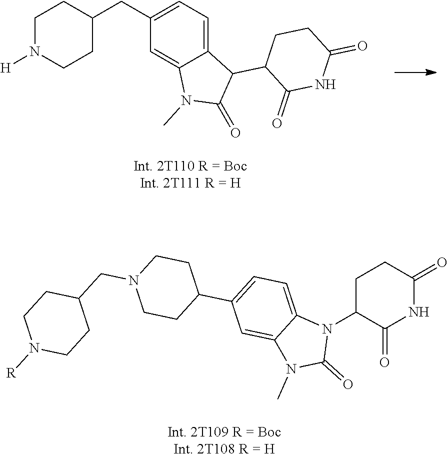

Int. 3U121 (1 g) and 10% Pd/C (0.5 g) was hydrogenated 16 h in MeOH (30 mL). The mixture was filtered and concentrated to give Int. 2T110 tert-butyl 4-((1-(2,6-dioxopiperidin-3-yl)-3-methyl-2-oxo-2,3-dihydro-1H-benzo[d]imidazol-5-yl)methyl)piperidine-1-carboxylate (0.80 g). Int. 2T110 (1.6 g) was stirred 4 h in DCM/4M HCl in dioxane (10:1, 22 mL) at 0° C. to RT. The residue after concentration was triturated in Et₂O to give Int. 2T111 3-(3-methyl-2-oxo-5-(piperidin-4-ylmethyl)-2,3-dihydro-1H-benzo[d]imidazol-1-yl)piperidine-2,6-dione (1.2 g, HCl salt). Int. 2T111 (1.20 g) and tert-butyl 4-formylpiperidine-1-carboxylate (0.50 g) were stirred in 16 h in DMSO (10 ml) and DIPEA (2.5 mL). STAB (1.98 g) was added and stirring continued 16 h at 0° C. to RT. The OL (EtOAc/ice-cold water) was washed with brine, dried, and concentrated. The residue was purified by FC (4% MeOH in DCM) to give Int. 2T109 tert-butyl 4-[[4-[[1-(2,6-dioxo-3-piperidyl)-3-methyl-2-oxo-benzimidazol-5-yl]methyl]-1-piperidyl]methyl]piperidine-1-carboxylate (0.45 g). Int. 2T109 (0.45 g) was stirred 4 h in DCM (15 mL) and 4M HCl in dioxane (5 mL) at 0° C. to RT. The residue after concentration was triturated with Et₂O to precipitate Int. 2T108 3-[3-methyl-2-oxo-5-(4-piperidylmethyl)benzimidazol-1-yl]piperidine-2,6-dione; hydrochloride (0.80 g).

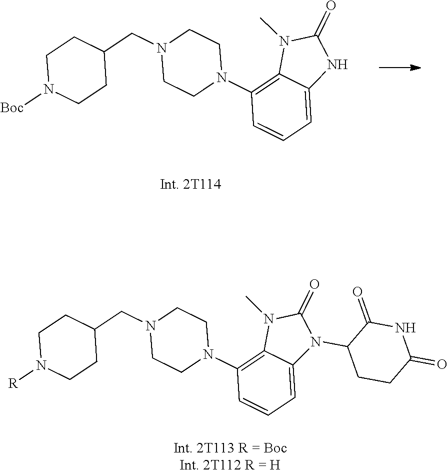

7-Bromo-1-methyl-1,3-dihydro-2H-benzo[d]imidazol-2-one (0.10 g), Int. 3E5 (0.16 g), RuPhos Pd G4 (37 mg) and RuPhos (41 mg) were stirred 5 h in toluene (10 mL) and 1M LiHMDS in THF (2.2 mL) at 0-80° C. under an inert atmosphere. The OL (EtOAc/water) was dried, concentrated, and purified by FC (50-70% of EtOAc in PE) to give Int. 2T114 tert-butyl 4-[[4-(3-methyl-2-oxo-1H-benzimidazol-4-yl)piperazin-1-yl]methyl]piperidine-1-carboxylate (0.48 g). Int. 2T114 (0.45 g) and 60% NaH (60% oil dispersion, 0.21 g) were stirred in THF (15 mL) at 0° C. to RT. 3-bromopiperidine-2,6-dione (1.0 g) was added and stirring continued 12 h at 60° C. The OL (EtOAc/ice-cold water) was concentrated. The residue was triturated with 50% MTBE in PE to precipitate Int. 2T113 tert-butyl 4-[[4-[1-(2,6-dioxo-3-piperidyl)-3-methyl-2-oxo-benzimidazol-4-yl]piperazin-1-yl]methyl]-piperidine-1-carboxylate (0.32 g). Int. 2T113 (0.3 g) was stirred 16 h in DCM/TFA (5 mL/2 mL) at 0° C. to RT. The residue after concentration was washed with Et₂O. The OL (10% MeOH in DCM/aq. NaHCO₃) was concentrated to give Int. 2T112 3-[3-methyl-2-oxo-4-[4-(4-piperidylmethyl)piperazin-1-yl]benzimidazol-1-yl]piperidine-2,6-dione (0.22 g).

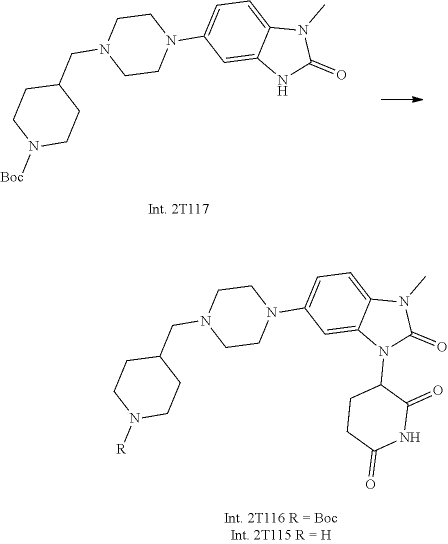

Int. 2T117 tert-butyl 4-[[4-(1-methyl-2-oxo-3H-benzimidazol-5-yl)piperazin-1-yl]methyl]piperidine-1-carboxylate (0.45 g) was prepared similarly to Int. 2T114 from Int. 3E5 (1.62 g) and 5-bromo-1-methyl-1,3-dihydro-2H-benzo[d]imidazol-2-one (1.0 g). Int. 2T117 (0.35 g) and NaH (60% oil dispersion, 0.33 g) were stirred 0.5 h in THF (150 mL) at 0-10° C. 3-Bromopiperidine-2,6-dione (0.78 g) was added at 0° C. and stirring was continued 16 h at 75-80° C. The OL (EtOAc/ice-cold water) was washed with brine, dried, and concentrated. The residue was purified washed with Et₂O/PE/DCM (1:2: 0.5) to precipitate Int. 2T116 tert-butyl 4-[[4-[3-(2,6-dioxo-3-piperidyl)-1-methyl-2-oxo-benzimidazol-5-yl]piperazin-1-yl]methyl]piperidine-1-carboxylate (0.28 g). Int. 2T116 (0.28 g) was stirred 5 h in DCM/TFA (5 mL/3 mL) at 0° C. to RT. The residue after concentration was washed with Et₂O and dried to give Int. 2T115 3-[3-methyl-2-oxo-6-[4-(4-piperidyl-methyl)piperazin-1-yl]benzimidazol-1-yl]piperidine-2,6-dione (0.25 g, TFA salt).

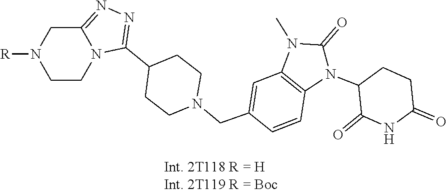

Int. 3U67 (0.20 g), tert-butyl 3-(piperidin-4-yl)-5,6-dihydro-[1,2,4]triazolo[4,3-a]pyrazine-7 (8H)-carboxylate (0.20 g), and 4A molecular sieves were stirred ON in DCE. NaCNBH₃ (51 mg) was added and stirring continued 3 h. The mixture was filtered and concentrated. The residue was HPLC-purified to give Int. 2T119 tert-butyl 3-[1-[[1-(2,6-dioxo-3-piperidyl)-3-methyl-2-oxo-benzimidazol-5-yl]methyl]-4-piperidyl]-6,8-dihydro-5H-[1,2,4]triazolo[4,3-a]pyrazine-7-carboxylate (52 mg). Int. 2T119 (52 mg) was stirred 1 h in HFIP (2 mL) concentrated aq. HCl (0.1 mL). The residue was diluted with TBME to precipitate Int. 2T118 3-[3-methyl-2-oxo-5-[[4-(5,6,7,8-tetrahydro-[1,2,4]triazolo[4,3-a]pyrazin-3-yl)-1-piperidyl]methyl]benzimidazol-1-yl]-piperidine-2,6-dione (34 mg, HCl salt).

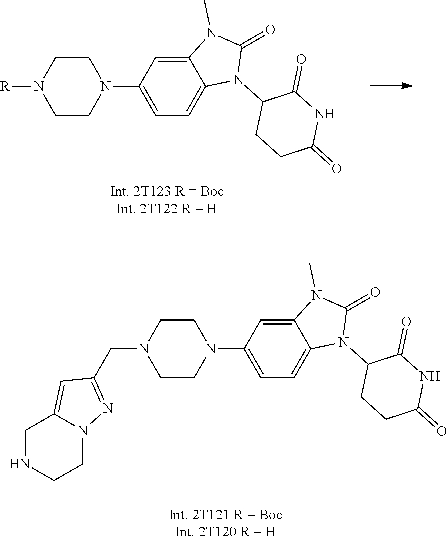

Int. 3E251 (1.0 g) and NaH (60% oil dispersion, 0.72 g) were stirred 0.3 h in THF (50 mL). 3-Bromopiperidine-2,6-dione (2.89 g) was added and stirring continued 14 h at 60° C. The OL (EtOAc/aq. NH₄C1) was concentrated to give Int. 2T123 tert-butyl 4-[1-(2,6-dioxo-3-piperidyl)-3-methyl-2-oxo-benzimidazol-5-yl]-piperazine-1-carboxylate (1.8 g). Int. 2T123 (1.0 g) was stirred 14 h in DCM/TFA (20 mL/0.95 mL). The residue after concentration was HPLC-purified to give Int. 2T122 3-(3-methyl-2-oxo-5-piperazin-1-yl-benzimidazol-1-yl)piperidine-2,6-dione (0.36 g). To solution of Int. 2T122 (0.36 g), tert-butyl 2-formyl-6,7-dihydropyrazolo-[1,5-a]pyrazine-5 (4H)-carboxylate (0.28 g) were stirred 0.3 h in DCM (30 mL). STAB (0.67 g) was added and stirring continued 11 h. The OL (DCM/sat. aq. NaHCO₃) was concentrated to give Int. 2T121 tert-butyl 2-[[4-[1-(2,6-dioxo-3-piperidyl)-3-methyl-2-oxo-benzimidazol-5-yl]piperazin-1-yl]methyl]-6,7-dihydro-4H-pyrazolo-[1,5-a]pyrazine-5-carboxylate (0.5 g). Int. 2T121 (0.50 g) was stirred 14 h in DCM/TFA (20 mL/0.33 mL). The residue after concentration was HPLC-purified to give Int. 2T120 3-[3-methyl-2-oxo-5-[4-(4,5,6,7-tetrahydro-pyrazolo[1,5-a]pyrazin-2-ylmethyl)piperazin-1-yl]benzimidazol-1-yl]piperidine-2,6-dione (0.19-g).

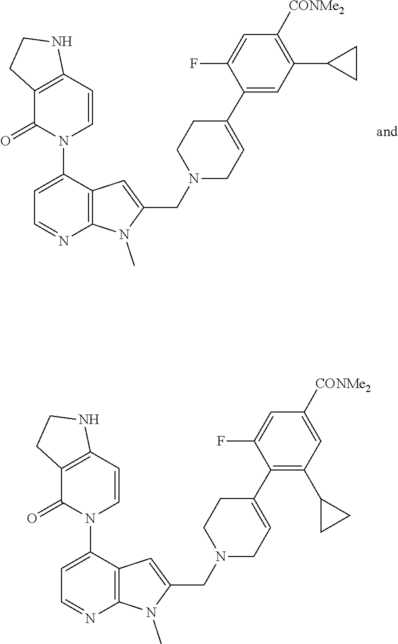

### Example 1m134. 2-cyclopropyl-5-fluoro-N,N-dimethyl-4-(1-((1-methyl-4-(4-oxo-1,2,3,4-tetrahydro-5H-pyrrolo[3,2-c]pyridin-5-yl)-1H-pyrrolo[2,3-b]pyridin-2-yl)methyl)-1,2,3,6-tetrahydropyridin-4-yl)benzamide and Example 1m135. 3-cyclopropyl-5-fluoro-N,N-dimethyl-4-(1-((1-methyl-4-(4-oxo-1,2,3,4-tetrahydro-5H-pyrrolo[3,2-c]pyridin-5-yl)-1H-pyrrolo[2,3-b]pyridin-2-yl)methyl)-1,2,3,6-tetrahydropyridin-4-yl)benzamide

CuI (6 mg), Ints. 1Z74/1Z123+1Z124 (59 mg/0.17 g), and K₂CO₃ (0.14 g) were stirred 16 h in DMSO (8 mL) and trans-N,N′-dimethylcyclohexane-1,2-diamine (11 μL) at 110° C. under an inert atmosphere. The mixture was HPLC-purified and resolved by HPLC using a Chiralpak IC 250×20 mm 5 μm column with an eluent of 50% IPA containing 0.1% Et₂NH and 50% MeOH containing 0.1% Et₂NH (12 mL/min) to give Example 1m134 (9 mg, first peak) and Example 1m135 (3 mg, second peak).

### Example 2—Preparation of Compounds of the Disclosure

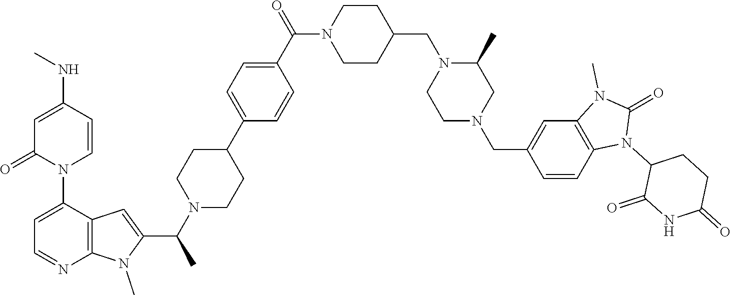 [SMILES: CNc1ccn(-c2ccnc3c2cc([C@H](C)N2CCC(c4ccc(C(=O)N5CCC(CN6CCN(Cc7ccc8c(c7)n(C)c(=O)n8C7CCC(=O)NC7=O)C[C@@H]6C)CC5)cc4)CC2)n3C)c(=O)c1]

### Example 3e39. 3-(3-methyl-5-(((S)-3-methyl-4-((1-(4-(1-((S)-1-(1-methyl-4-(4-(methylamino)-2-oxopyridin-1 (2H)-yl)-1H-pyrrolo[2,3-b]pyridin-2-yl)ethyl)piperidin-4-yl)benzoyl)piperidin-4-yl)methyl)piperazin-1-yl)methyl)-2-oxo-2,3-dihydro-1H-benzo[d]imidazol-1-yl)piperidine-2,6-dione

Ints. 3E171/3E68 (0.22 g/0.25 g) and PyBrop (0.3 g) were stirred ON in THF/DIPEA (37:1, 15.4 mL) at 0° C. to RT and diluted with water to precipitate a solid that was HPLC-purified to give Example 3e39 (48 mg).

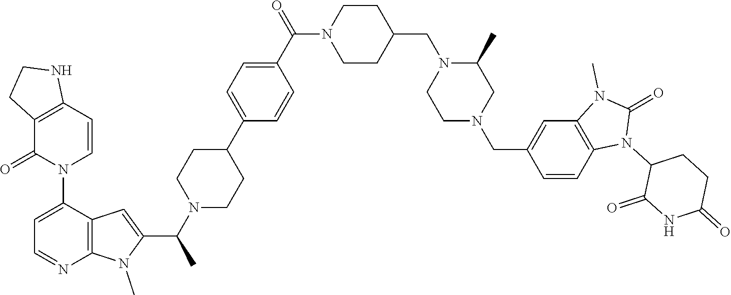 [SMILES: C[C@H]1CN(Cc2ccc3c(c2)n(C)c(=O)n3C2CCC(=O)NC2=O)CCN1CC1CCN(C(=O)c2ccc(C3CCN([C@@H](C)c4cc5c(-n6ccc7c(c6=O)CCN7)ccnc5n4C)CC3)cc2)CC1]

### Example 3e44. 3-(3-methyl-5-(((S)-3-methyl-4-((1-(4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)piperidin-4-yl)benzoyl)piperidin-4-yl)methyl)piperazin-1-yl)methyl)-2-oxo-2,3-dihydro-1H-benzo[d]imidazol-1-yl)piperidine-2,6-dione

Ints. 3E178/3E68 (0.15 g/0.13 g) and HATU (96 mg) were stirred ON in DMF/DIPEA (28:1, 5.2 mL) and diluted with water to precipitate a solid that was HPLC-purified to give Example 3e44 (14 mg).

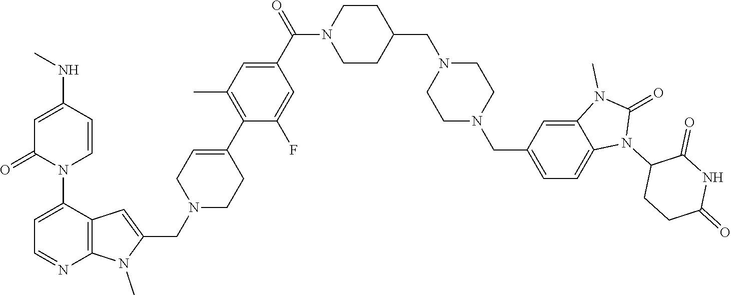 [SMILES: CNc1ccn(-c2ccnc3c2cc(CN2CC=C(c4c(C)cc(C(=O)N5CCC(CN6CCN(Cc7ccc8c(c7)n(C)c(=O)n8C7CCC(=O)NC7=O)CC6)CC5)cc4F)CC2)n3C)c(=O)c1]

### Example 3u163. 3-(5-((4-((1-(3-fluoro-5-methyl-4-(1-((1-methyl-4-(4-(methylamino)-2-oxopyridin-1 (2H)-yl)-1H-pyrrolo[2,3-b]pyridin-2-yl)methyl)-1,2,3,6-tetrahydropyridin-4-yl)benzoyl)piperidin-4-yl)methyl)piperazin-1-yl)methyl)-3-methyl-2-oxo-2,3-dihydro-1H-benzo[d]imidazol-1-yl)piperidine-2,6-dione

Ints. 1M60/3E1 (0.30 g/0.41 g) were mixed in DMF/Et₃N (6:1, 5.8 mL). T3P (50% in EtOAc, 0.53 mL) was added at 0° C. and stirred for 16 h at RT. The mixture was diluted with water to precipitate a solid that was HPLC-purified to give Example 3u163 (95 mg).

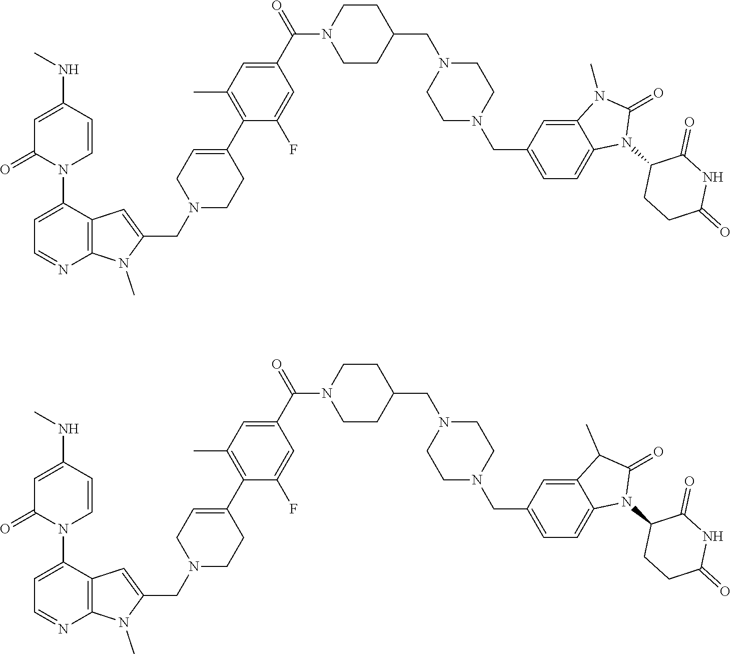 [SMILES: CNc1ccn(-c2ccnc3c2cc(CN2CC=C(c4c(C)cc(C(=O)N5CCC(CN6CCN(Cc7ccc8c(c7)n(C)c(=O)n8[C@H]7CCC(=O)NC7=O)CC6)CC5)cc4F)CC2)n3C)c(=O)c1 ; CNc1ccn(-c2ccnc3c2cc(CN2CC=C(c4c(C)cc(C(=O)N5CCC(CN6CCN(Cc7ccc8c(c7)C(C)C(=O)N8[C@@H]7CCC(=O)NC7=O)CC6)CC5)cc4F)CC2)n3C)c(=O)c1]

### Example 3u161 and 3u162. (S)-3-(5-((4-((1-(3-fluoro-5-methyl-4-(1-((1-methyl-4-(4-(methylamino)-2-oxopyridin-1 (2H)-yl)-1H-pyrrolo[2,3-b]pyridin-2-yl)methyl)-1,2,3,6-tetrahydropyridin-4-yl)benzoyl)-piperidin-4-yl)methyl)piperazin-1-yl)methyl)-3-methyl-2-oxo-2,3-dihydro-1H-benzo[d]imidazol-1-yl)piperidine-2,6-dione and (R)-3-(5-((4-((1-(3-fluoro-5-methyl-4-(1-((1-methyl-4-(4-(methylamino)-2-oxopyridin-1 (2H)-yl)-1H-pyrrolo[2,3-b]pyridin-2-yl)methyl)-1,2,3,6-tetrahydropyridin-4-yl)benzoyl)-piperidin-4-yl)methyl)piperazin-1-yl)methyl)-3-methyl-2-oxo-2,3-dihydro-1H-benzo[d]imidazol-1-yl)piperidine-2,6-dione

Example 3u163 (80 mg) was resolved by HPLC using a Chiralpak IK 250×30 mm 5 μm column with an eluent of 35% ACN, 35% IPA and 30% DCM (42 mL/min) to give Example 3u161 (31 mg, first peak) and Example 3u162 (23 mg, second peak). The absolute configurations for these compounds were not determined.

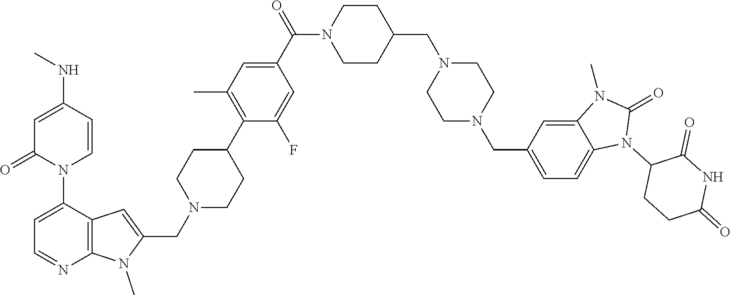 [SMILES: CNc1ccn(-c2ccnc3c2cc(CN2CCC(c4c(C)cc(C(=O)N5CCC(CN6CCN(Cc7ccc8c(c7)n(C)c(=O)n8C7CCC(=O)NC7=O)CC6)CC5)cc4F)CC2)n3C)c(=O)c1]

### Example 3u158. 3-(5-((4-((1-(3-fluoro-5-methyl-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)methyl)-piperazin-1-yl)methyl)-3-methyl-2-oxo-2,3-dihydro-1H-benzo[d]imidazol-1-yl)piperidine-2,6-dione

Example 3u158 (40 mg) was prepared similarly to Example 3u163 from Ints. 2G113/3E1 (0.30 g/0.41 g).

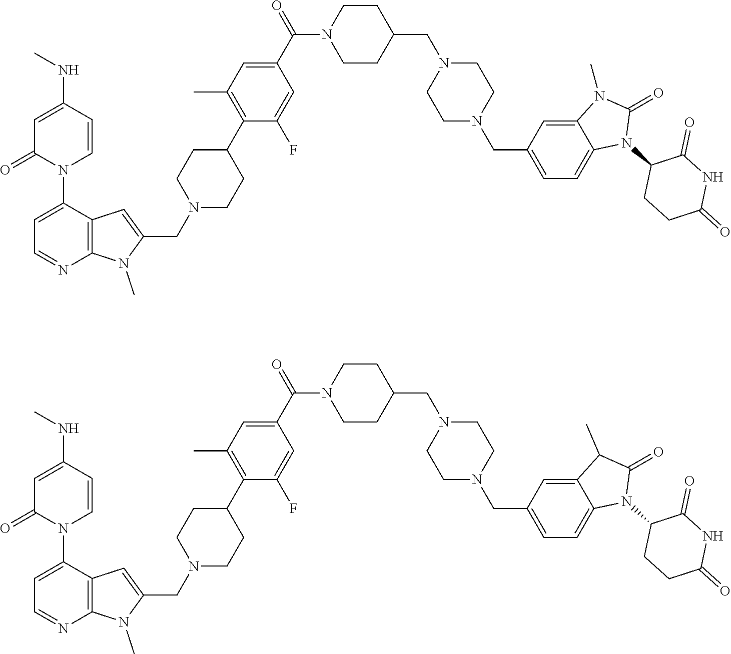 [SMILES: CNc1ccn(-c2ccnc3c2cc(CN2CCC(c4c(C)cc(C(=O)N5CCC(CN6CCN(Cc7ccc8c(c7)n(C)c(=O)n8[C@@H]7CCC(=O)NC7=O)CC6)CC5)cc4F)CC2)n3C)c(=O)c1 ; CNc1ccn(-c2ccnc3c2cc(CN2CCC(c4c(C)cc(C(=O)N5CCC(CN6CCN(Cc7ccc8c(c7)C(C)C(=O)N8[C@H]7CCC(=O)NC7=O)CC6)CC5)cc4F)CC2)n3C)c(=O)c1]

### Example 3u159 and 3u160. (R)-3-(5-((4-((1-(3-fluoro-5-methyl-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)methyl)piperazin-1-yl)methyl)-3-methyl-2-oxo-2,3-dihydro-1H-benzo[d]imidazol-1-yl)piperidine-2,6-dione and (S)-3-(5-((4-((1-(3-fluoro-5-methyl-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)methyl)piperazin-1-yl)methyl)-3-methyl-2-oxo-2,3-dihydro-1H-benzo[d]imidazol-1-yl)piperidine-2,6-dione

Example 2u158 (0.12 g) was resolved by HPLC using a Chiralpak IK 250×10 mm 5 μm column with an eluent of 35% ACN, 35% IPA and 30% DCM (4 mL/min) to give Example 3u160 (17 mg, first peak) and Example 3u159 (32 mg, second peak). The absolute configurations for these compounds were not determined.

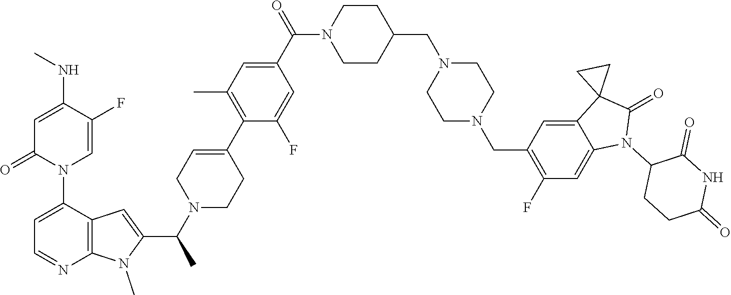 [SMILES: CNc1cc(=O)n(-c2ccnc3c2cc([C@H](C)N2CC=C(c4c(C)cc(C(=O)N5CCC(CN6CCN(Cc7cc8c(cc7F)N(C7CCC(=O)NC7=O)C(=O)C87CC7)CC6)CC5)cc4F)CC2)n3C)cc1F]

### Example 3u152. 3-(6′-fluoro-5′-((4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)methyl)-2′-oxospiro[cyclopropane-1,3′-indolin]-1′-yl)piperidine-2,6-dione

Example 3u152 (0.12 g) was prepared similarly to Example 3u163 from Ints. 2G45/3C4 (0.30 g/0.44 g).

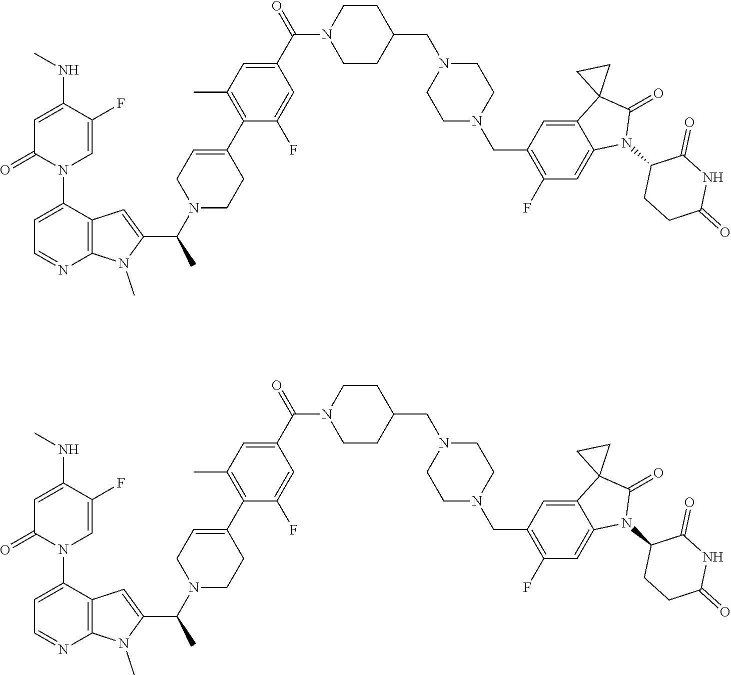 [SMILES: CNc1cc(=O)n(-c2ccnc3c2cc([C@H](C)N2CC=C(c4c(C)cc(C(=O)N5CCC(CN6CCN(Cc7cc8c(cc7F)N([C@H]7CCC(=O)NC7=O)C(=O)C87CC7)CC6)CC5)cc4F)CC2)n3C)cc1F ; CNc1cc(=O)n(-c2ccnc3c2cc([C@H](C)N2CC=C(c4c(C)cc(C(=O)N5CCC(CN6CCN(Cc7cc8c(cc7F)N([C@@H]7CCC(=O)NC7=O)C(=O)C87CC7)CC6)CC5)cc4F)CC2)n3C)cc1F]

### Example 3u154 and 3u153. (S)-3-(6′-fluoro-5′-((4-((1-(3-fluoro-4-(1-((S)-1-(4-(5-fluoro-4-(methyl-amino)-2-oxopyridin-1 (2H)-yl)-1-methyl-1H-pyrrolo[2,3-b]pyridin-2-yl)ethyl)-1,2,3,6-tetrahydro-pyridin-4-yl)-5-methylbenzoyl)piperidin-4-yl)methyl)piperazin-1-yl)methyl)-2′-oxospiro-[cyclopropane-1,3′-indolin]-1′-yl)piperidine-2,6-dione and (R)-3-(6′-fluoro-5′-((4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)methyl)-2′-oxospiro[cyclopropane-1,3′-indolin]-1′-yl)piperidine-2,6-dione

Example 3u152 (0.10 g) was resolved by HPLC using a CELLULOSE-SC 250×30 mm 5 μm column with an eluent of 35% ACN, 35% IPA and 30% DCM (26 mL/min) to give Example 3u153 (25 mg, first peak) and Example 3u154 (25 mg, second peak). The absolute configurations for these compounds at the glutarimide chiral centers were not determined.

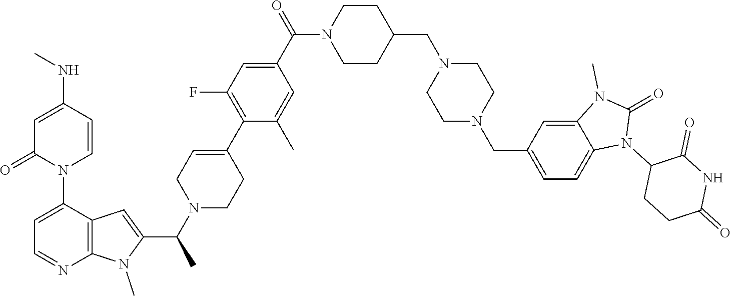 [SMILES: CNc1ccn(-c2ccnc3c2cc([C@H](C)N2CC=C(c4c(C)cc(C(=O)N5CCC(CN6CCN(Cc7ccc8c(c7)n(C)c(=O)n8C7CCC(=O)NC7=O)CC6)CC5)cc4F)CC2)n3C)c(=O)c1]

### Example 3u155. 3-(5-((4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)methyl)-3-methyl-2-oxo-2,3-dihydro-1H-benzo[d]imidazol-1-yl)piperidine-2,6-dione

Example 3u155 (0.19 g) was prepared similarly to Example 3u163 from Ints. 2G46/3E1 (0.30 g/0.40 g).

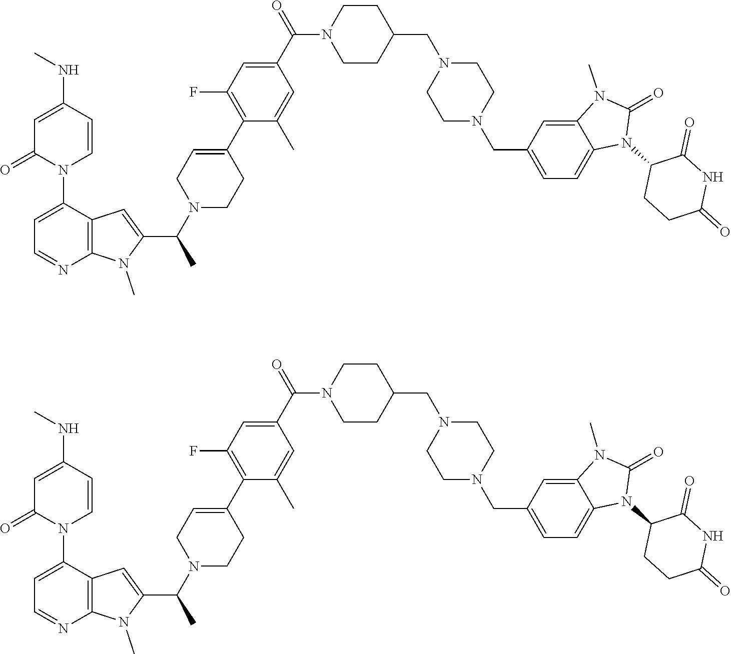 [SMILES: CNc1ccn(-c2ccnc3c2cc([C@H](C)N2CC=C(c4c(C)cc(C(=O)N5CCC(CN6CCN(Cc7ccc8c(c7)n(C)c(=O)n8[C@H]7CCC(=O)NC7=O)CC6)CC5)cc4F)CC2)n3C)c(=O)c1 ; CNc1ccn(-c2ccnc3c2cc([C@H](C)N2CC=C(c4c(C)cc(C(=O)N5CCC(CN6CCN(Cc7ccc8c(c7)n(C)c(=O)n8[C@@H]7CCC(=O)NC7=O)CC6)CC5)cc4F)CC2)n3C)c(=O)c1]

### Example 3u156 and 3u157. (S)-3-(5-((4-((1-(3-fluoro-5-methyl-4-(1-((S)-1-(1-methyl-4-(4-(methyl-amino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)methyl)-3-methyl-2-oxo-2,3-dihydro-1H-benzo[d]-imidazol-1-yl)piperidine-2,6-dione and (R)-3-(5-((4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)methyl)-3-methyl-2-oxo-2,3-dihydro-1H-benzo[d]-imidazol-1-yl)piperidine-2,6-dione

Example 3u155 (0.17 g) was resolved by HPLC using a Chiralpak IK 250×30 mm 5 μm column with an eluent of 35% ACN, 35% IPA and 30% DCM (38 mL/min) to give Example 3u157 (35 mg, first peak) and Example 3u156 (55 mg, second peak). The absolute configurations for these compounds at the glutarimide chiral centers were not determined.

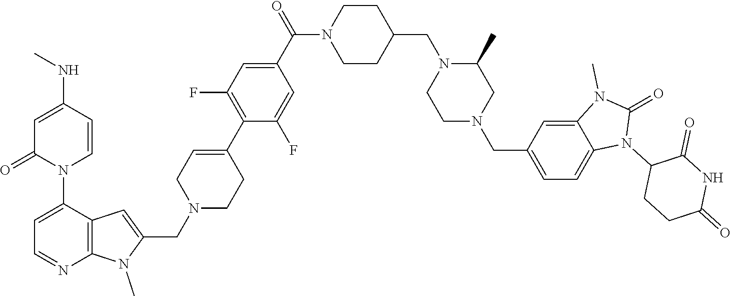 [SMILES: CNc1ccn(-c2ccnc3c2cc(CN2CC=C(c4c(F)cc(C(=O)N5CCC(CN6CCN(Cc7ccc8c(c7)n(C)c(=O)n8C7CCC(=O)NC7=O)C[C@@H]6C)CC5)cc4F)CC2)n3C)c(=O)c1]

### Example 3u1. 3-(5-(((S)-4-((1-(3,5-difluoro-4-(1-((1-methyl-4-(4-(methylamino)-2-oxopyridin-1 (2H)-yl)-1H-pyrrolo[2,3-b]pyridin-2-yl)methyl)-1,2,3,6-tetrahydropyridin-4-yl)benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione

Example 3u1 (85 mg) was prepared similarly to Example 3u163 from Ints. 3U90/3E68 (0.30 g/0.36 g).

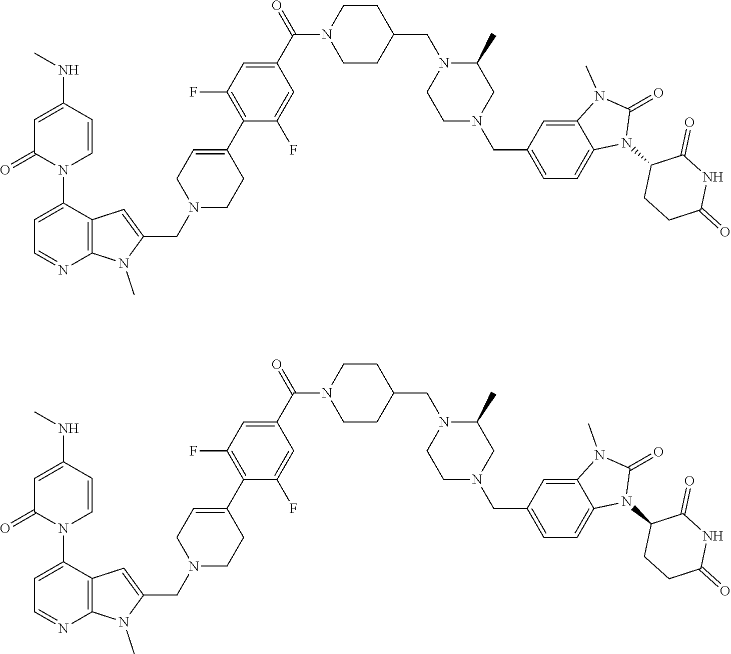 [SMILES: CNc1ccn(-c2ccnc3c2cc(CN2CC=C(c4c(F)cc(C(=O)N5CCC(CN6CCN(Cc7ccc8c(c7)n(C)c(=O)n8[C@H]7CCC(=O)NC7=O)C[C@@H]6C)CC5)cc4F)CC2)n3C)c(=O)c1 ; CNc1ccn(-c2ccnc3c2cc(CN2CC=C(c4c(F)cc(C(=O)N5CCC(CN6CCN(Cc7ccc8c(c7)n(C)c(=O)n8[C@@H]7CCC(=O)NC7=O)C[C@@H]6C)CC5)cc4F)CC2)n3C)c(=O)c1]

### Example 3u2 and 3u3. (S)-3-(5-(((S)-4-((1-(3,5-difluoro-4-(1-((1-methyl-4-(4-(methylamino)-2-oxopyridin-1 (2H)-yl)-1H-pyrrolo[2,3-b]pyridin-2-yl)methyl)-1,2,3,6-tetrahydropyridin-4-yl)benzoyl)-piperidin-4-yl)methyl)-3-methylpiperazin-1-yl)methyl)-3-methyl-2-oxo-2,3-dihydro-1H-benzo[d]-imidazol-1-yl)piperidine-2,6-dione and (R)-3-(5-(((S)-4-((1-(3,5-difluoro-4-(1-((1-methyl-4-(4-(methylamino)-2-oxopyridin-1 (2H)-yl)-1H-pyrrolo[2,3-b]pyridin-2-yl)methyl)-1,2,3,6-tetrahydro-pyridin-4-yl)benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione

Example 3u1 (65 mg) was resolved by HPLC using a Chiralpak IK 150×10 mm 5 μm column with an eluent of 35% ACN, 35% IPA and 30% DCM (4 mL/min) to give Example 3u2 (17 mg, first peak) and Example 3u3 (15 mg, second peak). The absolute configurations for these compounds at the glutarimide chiral centers were not determined.

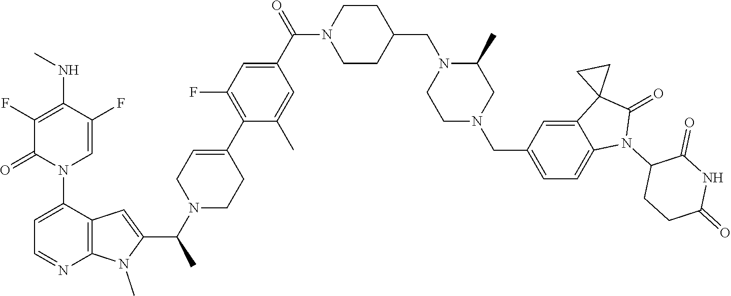 [SMILES: CNc1c(F)cn(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)c(=O)c1F]

### Example 3u4. 3-(5′-(((S)-4-((1-(4-(1-((S)-1-(4-(3,5-difluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-3-fluoro-5-methyl-benzoyl)-piperidin-4-yl)methyl)-3-methylpiperazin-1-yl)methyl)-2′-oxospiro[cyclopropane-1,3′-indolin]-1′-yl)piperidine-2,6-dione

Example 3u4 (21 mg) was prepared similarly to Example 3u163 from Ints. 3U4/3C42 (0.10 g/0.12 g).

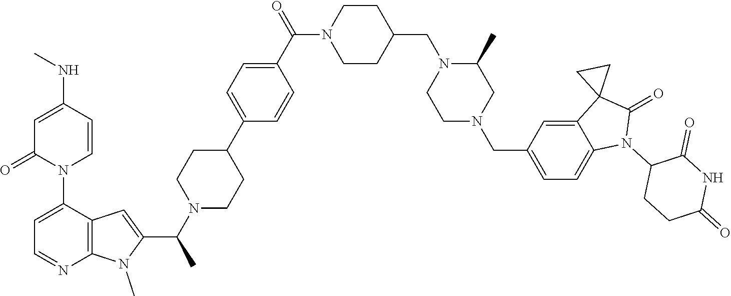 [SMILES: CNc1ccn(-c2ccnc3c2cc([C@H](C)N2CCC(c4ccc(C(=O)N5CCC(CN6CCN(Cc7ccc8c(c7)C7(CC7)C(=O)N8C7CCC(=O)NC7=O)C[C@@H]6C)CC5)cc4)CC2)n3C)c(=O)c1]

### Example 3u5. 3-(5′-(((S)-3-methyl-4-((1-(4-(1-((S)-1-(1-methyl-4-(4-(methylamino)-2-oxopyridin-1 (2H)-yl)-1H-pyrrolo[2,3-b]pyridin-2-yl)ethyl)piperidin-4-yl)benzoyl)piperidin-4-yl)methyl)piperazin-1-yl)-methyl)-2′-oxospiro[cyclopropane-1,3′-indolin]-1′-yl)piperidine-2,6-dione

Example 3u5 (0.10 g) was prepared similarly to Example 3u163 from Ints. 3E171/3C42 (0.28 g/0.41 g).

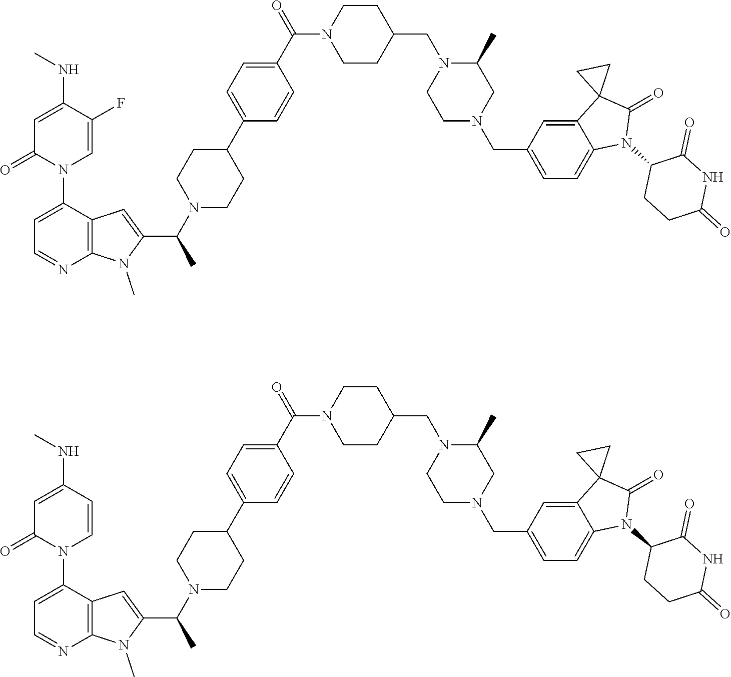 [SMILES: CNc1cc(=O)n(-c2ccnc3c2cc([C@H](C)N2CCC(c4ccc(C(=O)N5CCC(CN6CCN(Cc7ccc8c(c7)C7(CC7)C(=O)N8[C@H]7CCC(=O)NC7=O)C[C@@H]6C)CC5)cc4)CC2)n3C)cc1F ; CNc1ccn(-c2ccnc3c2cc([C@H](C)N2CCC(c4ccc(C(=O)N5CCC(CN6CCN(Cc7ccc8c(c7)C7(CC7)C(=O)N8[C@@H]7CCC(=O)NC7=O)C[C@@H]6C)CC5)cc4)CC2)n3C)c(=O)c1]

### Example 3u6 and 3u7. (S)-3-(5′-(((S)-3-methyl-4-((1-(4-(1-((S)-1-(1-methyl-4-(4-(methylamino)-2-oxopyridin-1 (2H)-yl)-1H-pyrrolo[2,3-b]pyridin-2-yl)ethyl)piperidin-4-yl)benzoyl)piperidin-4-yl)methyl)-piperazin-1-yl)methyl)-2′-oxospiro[cyclopropane-1,3′-indolin]-1′-yl)piperidine-2,6-dione and (R)-3-(5′-(((S)-3-methyl-4-((1-(4-(1-((S)-1-(1-methyl-4-(4-(methylamino)-2-oxopyridin-1 (2H)-yl)-1H-pyrrolo[2,3-b]pyridin-2-yl)ethyl)piperidin-4-yl)benzoyl)piperidin-4-yl)methyl)piperazin-1-yl)methyl)-2′-oxospiro-[cyclopropane-1,3′-indolin]-1′-yl)piperidine-2,6-dione

Example 3u5 (0.14 g) was resolved by HPLC using a Chiralpak IK 250×30 mm 5 μm column with an eluent of 35% ACN, 35% IPA and 30% DCM (37 mL/min) to give Example 3u6 (8 mg, first peak) and Example 3u7 (10 mg, second peak). The absolute configurations for these compounds at the glutarimide chiral centers were not determined.

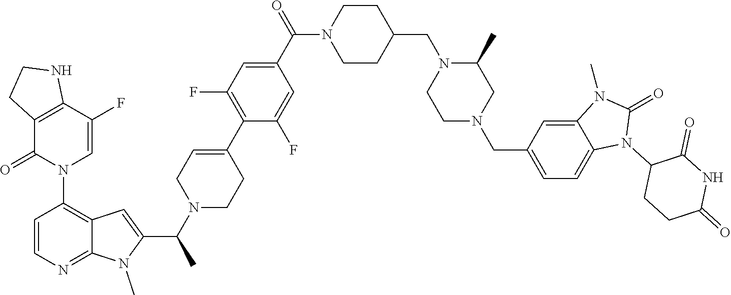 [SMILES: C[C@H]1CN(Cc2ccc3c(c2)n(C)c(=O)n3C2CCC(=O)NC2=O)CCN1CC1CCN(C(=O)c2cc(F)c(C3=CCN([C@@H](C)c4cc5c(-n6cc(F)c7c(c6=O)CCN7)ccnc5n4C)CC3)c(F)c2)CC1]

### Example 3u8. 3-(5-(((S)-4-((1-(3,5-difluoro-4-(1-((S)-1-(4-(7-fluoro-4-oxo-1,2,3,4-tetrahydro-5H-pyrrolo[3,2-c]pyridin-5-yl)-1-methyl-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione

Example 3u8 (15 mg) was prepared similarly to Example 3u163 from Ints. 2G11/3E68 (0.10 g/0.12 g).

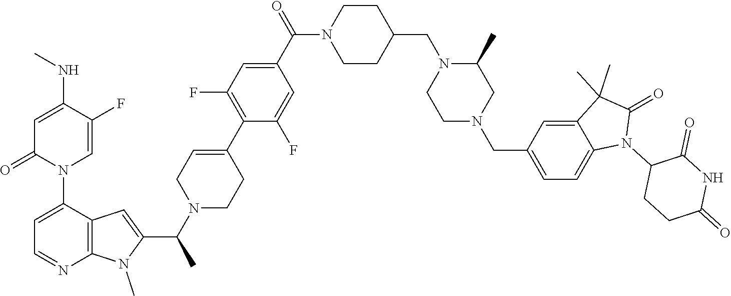 [SMILES: CNc1cc(=O)n(-c2ccnc3c2cc([C@H](C)N2CC=C(c4c(F)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)cc1F]

### Example 3u9. 3-(5-(((S)-4-((1-(3,5-difluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)benzoyl)-piperidin-4-yl)methyl)-3-methylpiperazin-1-yl)methyl)-3,3-dimethyl-2-oxoindolin-1-yl)piperidine-2,6-dione

Example 3u9 (15 mg) was prepared similarly to Example 3u163 from Ints. 2G43/3E291 (0.20 g/0.22 g).

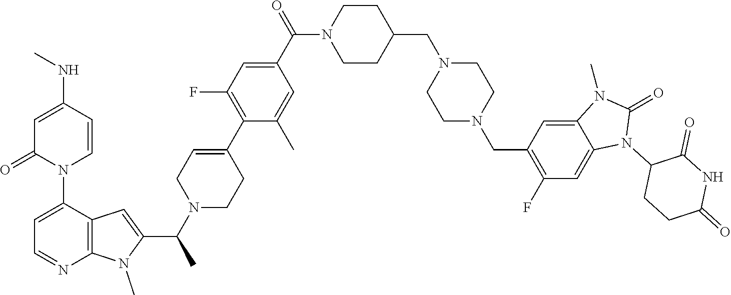 [SMILES: CNc1ccn(-c2ccnc3c2cc([C@H](C)N2CC=C(c4c(C)cc(C(=O)N5CCC(CN6CCN(Cc7cc8c(cc7F)n(C7CCC(=O)NC7=O)c(=O)n8C)CC6)CC5)cc4F)CC2)n3C)c(=O)c1]

### Example 3u10. 3-(6-fluoro-5-((4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)-piperidin-4-yl)methyl)piperazin-1-yl)methyl)-3-methyl-2-oxo-2,3-dihydro-1H-benzo[d]imidazol-1-yl)piperidine-2,6-dione

Example 3u10 (0.14 g) was prepared similarly to Example 3u163 from Ints. 2G46/3E145 (0.25 g/0.35 g).

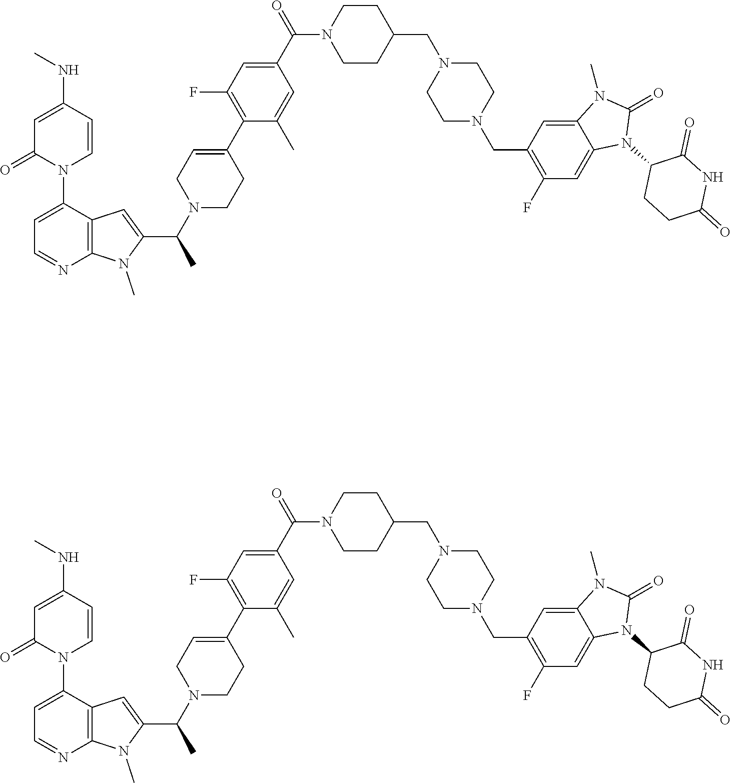 [SMILES: CNc1ccn(-c2ccnc3c2cc([C@H](C)N2CC=C(c4c(C)cc(C(=O)N5CCC(CN6CCN(Cc7cc8c(cc7F)n([C@H]7CCC(=O)NC7=O)c(=O)n8C)CC6)CC5)cc4F)CC2)n3C)c(=O)c1 ; CNc1ccn(-c2ccnc3c2cc([C@H](C)N2CC=C(c4c(C)cc(C(=O)N5CCC(CN6CCN(Cc7cc8c(cc7F)n([C@@H]7CCC(=O)NC7=O)c(=O)n8C)CC6)CC5)cc4F)CC2)n3C)c(=O)c1]

### Example 3u11 and 3u12. (S)-3-(6-fluoro-5-((4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)methyl)-3-methyl-2-oxo-2,3-dihydro-1H-benzo[d]-imidazol-1-yl)piperidine-2,6-dione and (R)-3-(6-fluoro-5-((4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)methyl)-3-methyl-2-oxo-2,3-dihydro-1H-benzo[d]imidazol-1-yl)piperidine-2,6-dione

Example 3u10 (0.12 g) was resolved by HPLC using a Chiralpak IK 250×30 mm 5 μm column with an eluent of 35% ACN, 35% IPA and 30% DCM (30 mL/min) to give Example 3u11 (42 mg, first peak) and Example 3u12 (31 mg, second peak). The absolute configurations for these compounds at the glutarimide chiral centers were not determined.

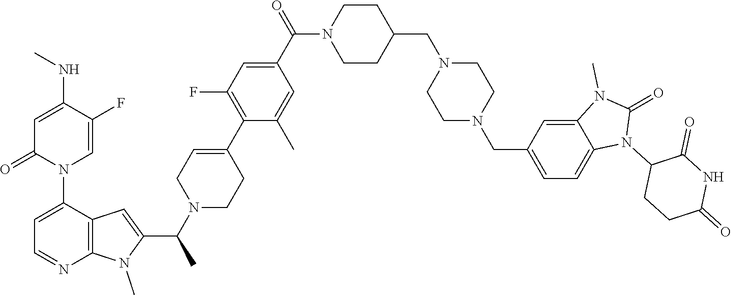 [SMILES: CNc1cc(=O)n(-c2ccnc3c2cc([C@H](C)N2CC=C(c4c(C)cc(C(=O)N5CCC(CN6CCN(Cc7ccc8c(c7)n(C)c(=O)n8C7CCC(=O)NC7=O)CC6)CC5)cc4F)CC2)n3C)cc1F]

### Example 3u13. 3-(5-((4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)-piperidin-4-yl)methyl)piperazin-1-yl)methyl)-3-methyl-2-oxo-2,3-dihydro-1H-benzo[d]imidazol-1-yl)piperidine-2,6-dione

Example 3u13 (0.16 g) was prepared similarly to Example 3u163 from Ints. 2G45/3E1 (0.30 g/0.41 g).

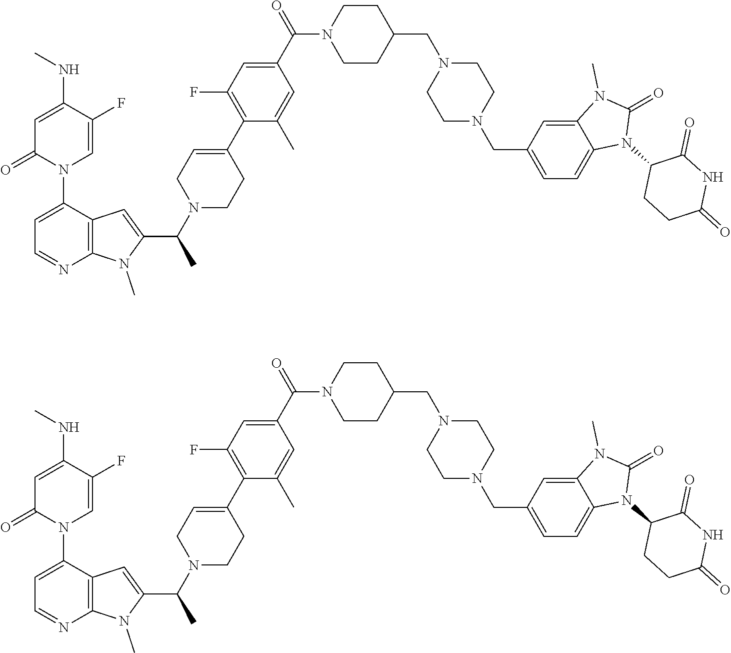 [SMILES: CNc1cc(=O)n(-c2ccnc3c2cc([C@H](C)N2CC=C(c4c(C)cc(C(=O)N5CCC(CN6CCN(Cc7ccc8c(c7)n(C)c(=O)n8[C@H]7CCC(=O)NC7=O)CC6)CC5)cc4F)CC2)n3C)cc1F ; CNc1cc(=O)n(-c2ccnc3c2cc([C@H](C)N2CC=C(c4c(C)cc(C(=O)N5CCC(CN6CCN(Cc7ccc8c(c7)n(C)c(=O)n8[C@@H]7CCC(=O)NC7=O)CC6)CC5)cc4F)CC2)n3C)cc1F]

### Example 3u14 and 3u15. (S)-3-(5-((4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)methyl)-3-methyl-2-oxo-2,3-dihydro-1H-benzo[d]imidazol-1-yl)piperidine-2,6-dione and (R)-3-(5-((4-((1-(3-fluoro-4-(1—((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)methyl)-3-methyl-2-oxo-2,3-dihydro-1H-benzo[d]imidazol-1-yl)piperidine-2,6-dione

Example 3u13 (0.13 g) was resolved by HPLC using a Chiralpak IK 250×30 mm 5 μm column with an eluent of 35% ACN, 35% IPA and 30% DCM (40 mL/min) to give Example 3u14 (31 mg, first peak) and Example 3u15 (21 mg, second peak). The absolute configurations for these compounds at the glutarimide chiral centers were not determined.

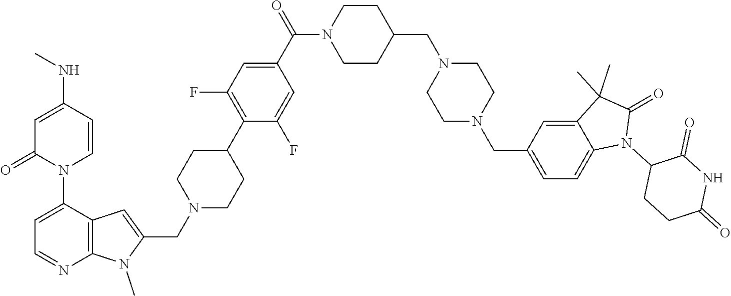 [SMILES: CNc1ccn(-c2ccnc3c2cc(CN2CCC(c4c(F)cc(C(=O)N5CCC(CN6CCN(Cc7ccc8c(c7)C(C)(C)C(=O)N8C7CCC(=O)NC7=O)CC6)CC5)cc4F)CC2)n3C)c(=O)c1]

### Example 3u16. 3-(5-((4-((1-(3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)methyl)piperazin-1-yl)-methyl)-3,3-dimethyl-2-oxoindolin-1-yl)piperidine-2,6-dione

Example 3u16 (64 mg) was prepared similarly to Example 3u163 from Ints. 2G108/3U7 (0.27 g/0.43 g).

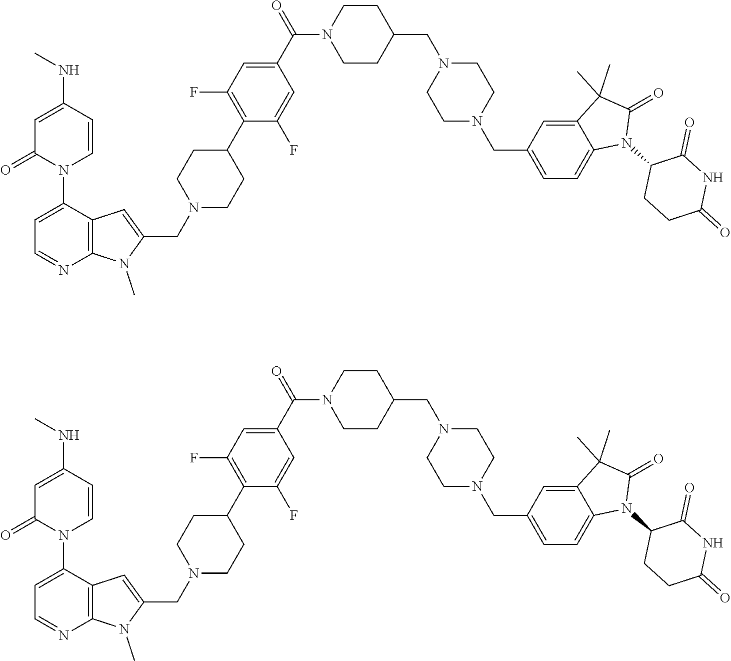 [SMILES: CNc1ccn(-c2ccnc3c2cc(CN2CCC(c4c(F)cc(C(=O)N5CCC(CN6CCN(Cc7ccc8c(c7)C(C)(C)C(=O)N8[C@H]7CCC(=O)NC7=O)CC6)CC5)cc4F)CC2)n3C)c(=O)c1 ; CNc1ccn(-c2ccnc3c2cc(CN2CCC(c4c(F)cc(C(=O)N5CCC(CN6CCN(Cc7ccc8c(c7)C(C)(C)C(=O)N8[C@@H]7CCC(=O)NC7=O)CC6)CC5)cc4F)CC2)n3C)c(=O)c1]

### Example 3u17 and 3u18. (S)-3-(5-((4-((1-(3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)-methyl)piperazin-1-yl)methyl)-3,3-dimethyl-2-oxoindolin-1-yl)piperidine-2,6-dione and (R)-3-(5-((4-((1-(3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)methyl)piperazin-1-yl)methyl)-3,3-dimethyl-2-oxoindolin-1-yl)piperidine-2,6-dione

Example 3u16 (48 mg) was resolved by HPLC using a Chiralpak IK 250×10 mm 5 μm column with an eluent of 35% ACN, 35% IPA and 30% DCM (3 mL/min) to give Example 3u17 (9 mg, TFA salt, first peak) and Example 3u18 (12 mg, TFA salt, second peak). The absolute configurations for these compounds at the glutarimide chiral centers were not determined.

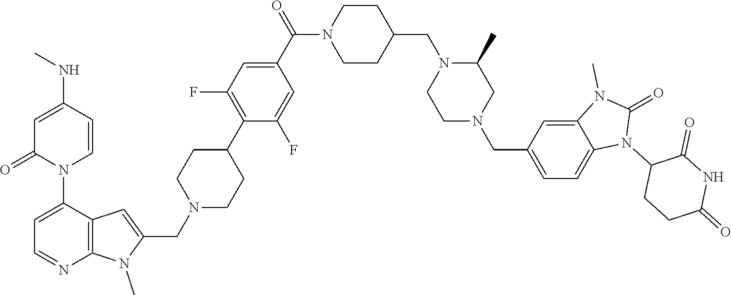 [SMILES: CNc1ccn(-c2ccnc3c2cc(CN2CCC(c4c(F)cc(C(=O)N5CCC(CN6CCN(Cc7ccc8c(c7)n(C)c(=O)n8C7CCC(=O)NC7=O)C[C@@H]6C)CC5)cc4F)CC2)n3C)c(=O)c1]

### Example 3u19. 3-(5-(((S)-4-((1-(3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)methyl)-3-methyl-piperazin-1-yl)methyl)-3-methyl-2-oxo-2,3-dihydro-1H-benzo[d]imidazol-1-yl)piperidine-2,6-dione

Example 3u19 (80 mg) was prepared similarly to Example 3u163 from Ints. 2G108/3E68 (0.30 g/0.38 g).

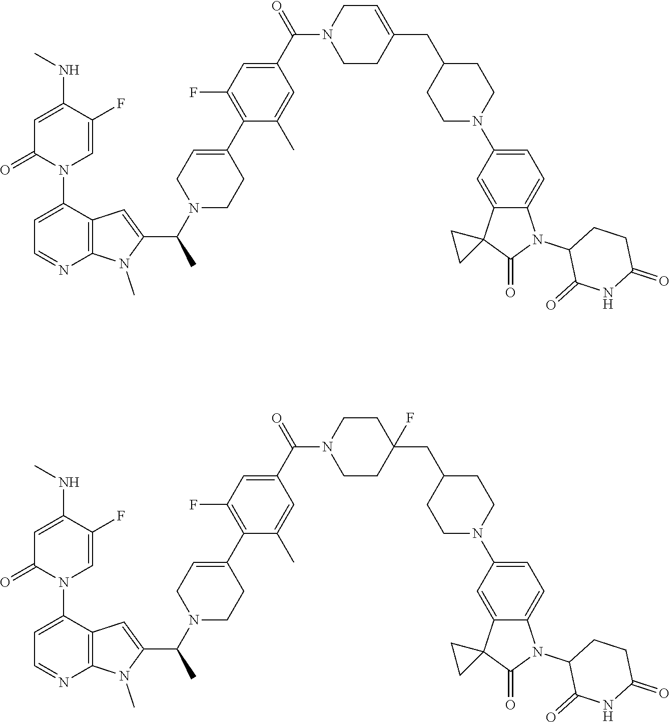 [SMILES: CNc1cc(=O)n(-c2ccnc3c2cc([C@H](C)N2CC=C(c4c(C)cc(C(=O)N5CC=C(CC6CCN(c7ccc8c(c7)C7(CC7)C(=O)N8C7CCC(=O)NC7=O)CC6)CC5)cc4F)CC2)n3C)cc1F ; CNc1cc(=O)n(-c2ccnc3c2cc([C@H](C)N2CC=C(c4c(C)cc(C(=O)N5CCC(F)(CC6CCN(c7ccc8c(c7)C7(CC7)C(=O)N8C7CCC(=O)NC7=O)CC6)CC5)cc4F)CC2)n3C)cc1F]

### Example 3u20 and 3u21. 3-(5′-(4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methyl-benzoyl)-1,2,3,6-tetrahydropyridin-4-yl)methyl)piperidin-1-yl)-2′-oxospiro[cyclopropane-1,3′-indolin]-1′-yl)piperidine-2,6-dione and 3-(5′-(4-((4-fluoro-1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperidin-1-yl)-2′-oxospiro[cyclopropane-1,3′-indolin]-1′-yl)-piperidine-2,6-dione

Examples 3u20 (63 mg) 3u21 (60 mg) were prepared similarly to Example 3u163 from Ints. 2G45/3U12/3U13 (0.30 g/0.44 g).

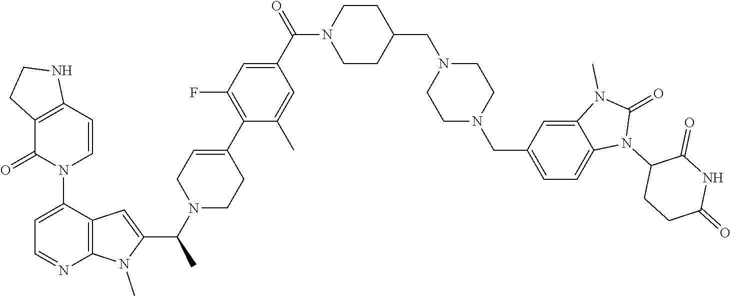 [SMILES: Cc1cc(C(=O)N2CCC(CN3CCN(Cc4ccc5c(c4)n(C)c(=O)n5C4CCC(=O)NC4=O)CC3)CC2)cc(F)c1C1=CCN([C@@H](C)c2cc3c(-n4ccc5c(c4=O)CCN5)ccnc3n2C)CC1]

### Example 3u22. 3-(5-((4-((1-(3-fluoro-5-methyl-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)-benzoyl)piperidin-4-yl)methyl)piperazin-1-yl)methyl)-3-methyl-2-oxo-2,3-dihydro-1H-benzo[d]-imidazol-1-yl)piperidine-2,6-dione

Example 3u22 (30 mg) was prepared similarly to Example 3u163 from Ints. 2G53/3E1 (0.15 g/0.16 g).

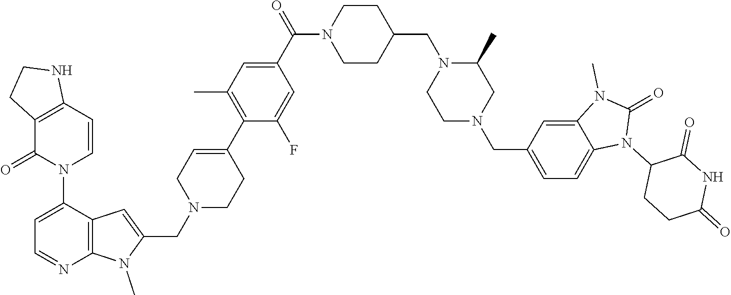 [SMILES: Cc1cc(C(=O)N2CCC(CN3CCN(Cc4ccc5c(c4)n(C)c(=O)n5C4CCC(=O)NC4=O)C[C@@H]3C)CC2)cc(F)c1C1=CCN(Cc2cc3c(-n4ccc5c(c4=O)CCN5)ccnc3n2C)CC1]

### Example 3u23. 3-(5-(((S)-4-((1-(3-fluoro-5-methyl-4-(1-((1-methyl-4-(4-oxo-1,2,3,4-tetrahydro-5H-pyrrolo[3,2-c]pyridin-5-yl)-1H-pyrrolo[2,3-b]pyridin-2-yl)methyl)-1,2,3,6-tetrahydropyridin-4-yl)benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione

Example 3u23 (0.10 g) was prepared similarly to Example 3u163 from Ints. 2G49/3E68 (0.30 g/0.29 g).

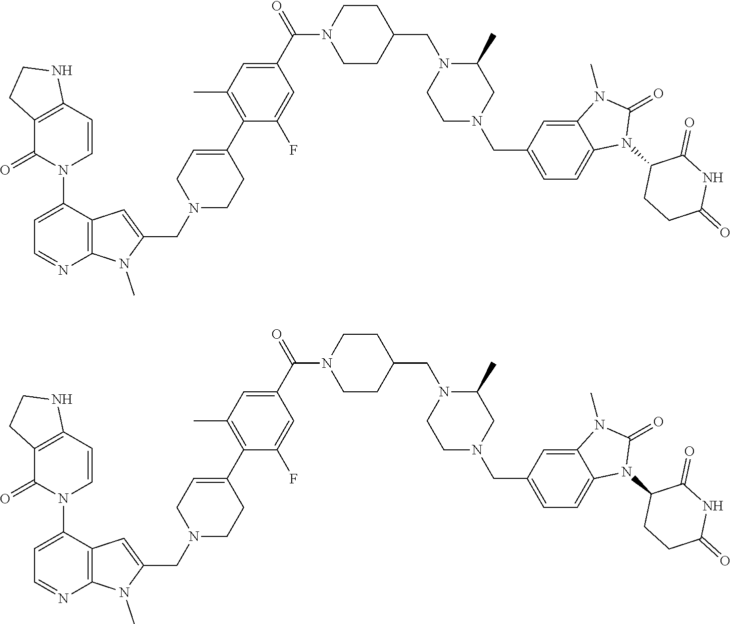 [SMILES: Cc1cc(C(=O)N2CCC(CN3CCN(Cc4ccc5c(c4)n(C)c(=O)n5[C@H]4CCC(=O)NC4=O)C[C@@H]3C)CC2)cc(F)c1C1=CCN(Cc2cc3c(-n4ccc5c(c4=O)CCN5)ccnc3n2C)CC1 ; Cc1cc(C(=O)N2CCC(CN3CCN(Cc4ccc5c(c4)n(C)c(=O)n5[C@@H]4CCC(=O)NC4=O)C[C@@H]3C)CC2)cc(F)c1C1=CCN(Cc2cc3c(-n4ccc5c(c4=O)CCN5)ccnc3n2C)CC1]

### Example 3u24 and 3u25. (S)-3-(5-(((S)-4-((1-(3-fluoro-5-methyl-4-(1-((1-methyl-4-(4-oxo-1,2,3,4-tetrahydro-5H-pyrrolo[3,2-c]pyridin-5-yl)-1H-pyrrolo[2,3-b]pyridin-2-yl)methyl)-1,2,3,6-tetrahydro-pyridin-4-yl)benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione and (R)-3-(5-(((S)-4-((1-(3-fluoro-5-methyl-4-(1-((1-methyl-4-(4-oxo-1,2,3,4-tetrahydro-5H-pyrrolo[3,2-c]pyridin-5-yl)-1H-pyrrolo[2,3-b]pyridin-2-yl)methyl)-1,2,3,6-tetrahydropyridin-4-yl)benzoyl)piperidin-4-yl)methyl)-3-methylpiperazin-1-yl)-methyl)-3-methyl-2-oxo-2,3-dihydro-1H-benzo[d]imidazol-1-yl)piperidine-2,6-dione

Example 3u23 (80 mg) was resolved by HPLC using a Chiralpak IK 250×10 mm 5 μm column with an eluent of 35% ACN, 35% IPA and 30% DCM (5 mL/min) to give Example 3u24 (15 mg, first peak) and Example 3u25 (18 mg, second peak). The absolute configurations for these compounds at the glutarimide chiral centers were not determined.

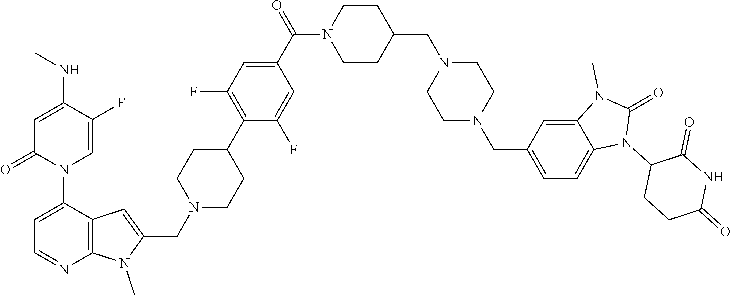 [SMILES: CNc1cc(=O)n(-c2ccnc3c2cc(CN2CCC(c4c(F)cc(C(=O)N5CCC(CN6CCN(Cc7ccc8c(c7)n(C)c(=O)n8C7CCC(=O)NC7=O)CC6)CC5)cc4F)CC2)n3C)cc1F]

### Example 3u26. 3-(5-((4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)methyl)piperazin-1-yl)methyl)-3-methyl-2-oxo-2,3-dihydro-1H-benzo[d]imidazol-1-yl)piperidine-2,6-dione

Example 3u26 (0.11 g) was prepared similarly to Example 3u163 from Ints. 2G58/3E1 (0.25 g/0.30 g).

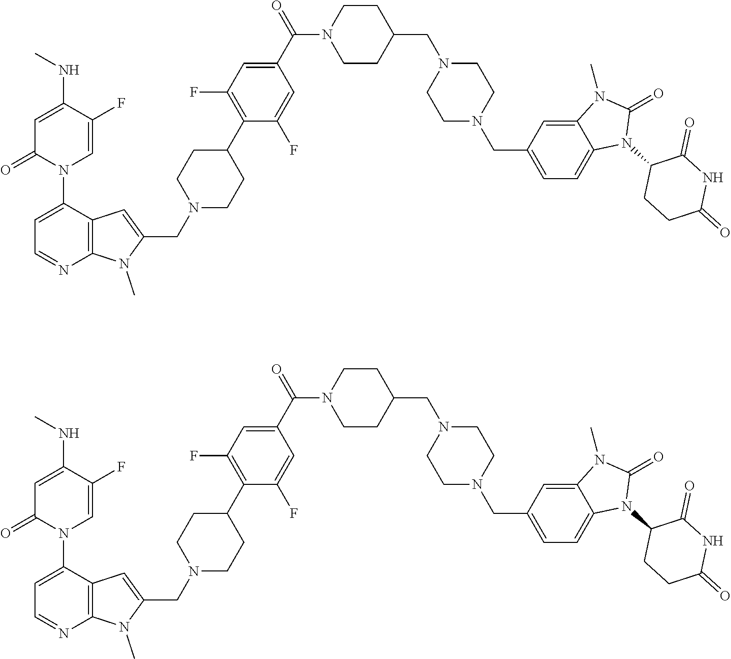 [SMILES: CNc1cc(=O)n(-c2ccnc3c2cc(CN2CCC(c4c(F)cc(C(=O)N5CCC(CN6CCN(Cc7ccc8c(c7)n(C)c(=O)n8[C@H]7CCC(=O)NC7=O)CC6)CC5)cc4F)CC2)n3C)cc1F ; CNc1cc(=O)n(-c2ccnc3c2cc(CN2CCC(c4c(F)cc(C(=O)N5CCC(CN6CCN(Cc7ccc8c(c7)n(C)c(=O)n8[C@@H]7CCC(=O)NC7=O)CC6)CC5)cc4F)CC2)n3C)cc1F]

### Example 3u27 and 3u28. (S)-3-(5-((4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)methyl)piperazin-1-yl)methyl)-3-methyl-2-oxo-2,3-dihydro-1H-benzo[d]imidazol-1-yl)piperidine-2,6-dione and (R)-3-(5-((4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)methyl)-piperazin-1-yl)methyl)-3-methyl-2-oxo-2,3-dihydro-1H-benzo[d]imidazol-1-yl)piperidine-2,6-dione

Example 3u26 (90 mg) was resolved by HPLC using a Chiralpak IK 250×30 mm 5 μm column with an eluent of 35% ACN, 35% IPA and 30% DCM (28 mL/min) to give Example 3u27 (11 mg, first peak) and Example 3u28 (19 mg, second peak). The absolute configurations for these compounds were not determined.

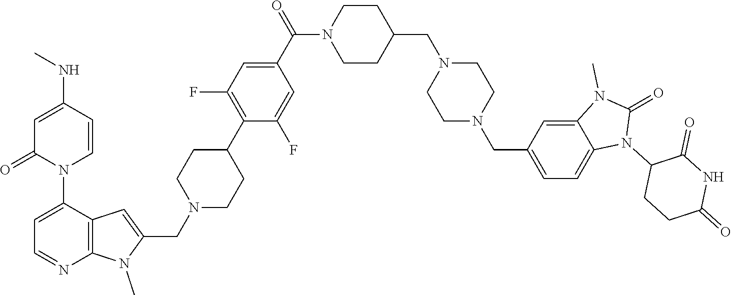 [SMILES: CNc1ccn(-c2ccnc3c2cc(CN2CCC(c4c(F)cc(C(=O)N5CCC(CN6CCN(Cc7ccc8c(c7)n(C)c(=O)n8C7CCC(=O)NC7=O)CC6)CC5)cc4F)CC2)n3C)c(=O)c1]

### Example 3u29. 3-(5-((4-((1-(3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)methyl)piperazin-1-yl)methyl)-3-methyl-2-oxo-2,3-dihydro-1H-benzo[d]imidazol-1-yl)piperidine-2,6-dione

Example 3u29 (60 mg) was prepared similarly to Example 3u163 from Ints. 2G108/3E1 (0.25 g/0.29 g).

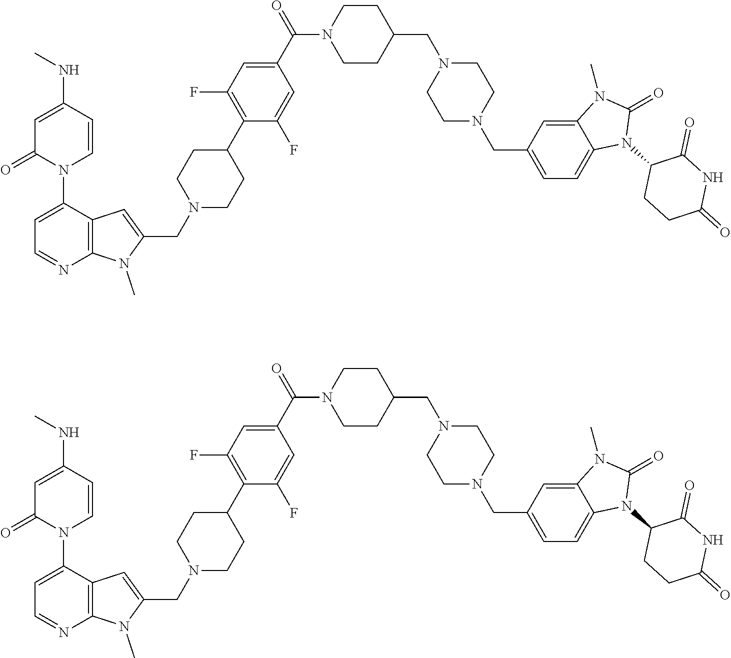 [SMILES: CNc1ccn(-c2ccnc3c2cc(CN2CCC(c4c(F)cc(C(=O)N5CCC(CN6CCN(Cc7ccc8c(c7)n(C)c(=O)n8[C@H]7CCC(=O)NC7=O)CC6)CC5)cc4F)CC2)n3C)c(=O)c1 ; CNc1ccn(-c2ccnc3c2cc(CN2CCC(c4c(F)cc(C(=O)N5CCC(CN6CCN(Cc7ccc8c(c7)n(C)c(=O)n8[C@@H]7CCC(=O)NC7=O)CC6)CC5)cc4F)CC2)n3C)c(=O)c1]

### Example 3u30 and 3u31. (S)-3-(5-((4-((1-(3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)methyl)piperazin-1-yl)methyl)-3-methyl-2-oxo-2,3-dihydro-1H-benzo[d]imidazol-1-yl)piperidine-2,6-dione and (R)-3-(5-((4-((1-(3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)methyl)piperazin-1-yl)methyl)-3-methyl-2-oxo-2,3-dihydro-1H-benzo[d]imidazol-1-yl)piperidine-2,6-dione

Example 3u29 (95 mg) was resolved by HPLC using a Chiralpak IK 250×30 mm 5 μm column with an eluent of 35% ACN, 35% IPA and 30% DCM (26 mL/min) to give Example 3u30 (25 mg, first peak) and Example 3u31 (25 mg, second peak). The absolute configurations for these compounds were not determined.

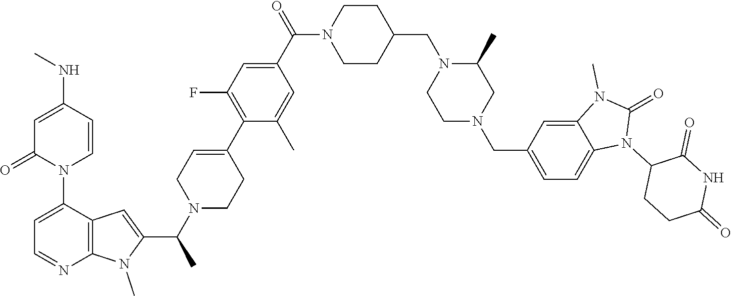 [SMILES: CNc1ccn(-c2ccnc3c2cc([C@H](C)N2CC=C(c4c(C)cc(C(=O)N5CCC(CN6CCN(Cc7ccc8c(c7)n(C)c(=O)n8C7CCC(=O)NC7=O)C[C@@H]6C)CC5)cc4F)CC2)n3C)c(=O)c1]

### Example 3u32. 3-(5-(((S)-4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)-piperidin-4-yl)methyl)-3-methylpiperazin-1-yl)methyl)-3-methyl-2-oxo-2,3-dihydro-1H-benzo[d]-imidazol-1-yl)piperidine-2,6-dione

Example 3u32 (0.14 g) was prepared similarly to Example 3u163 from Ints. 2G46/3E68 (0.30 g/0.41 g).

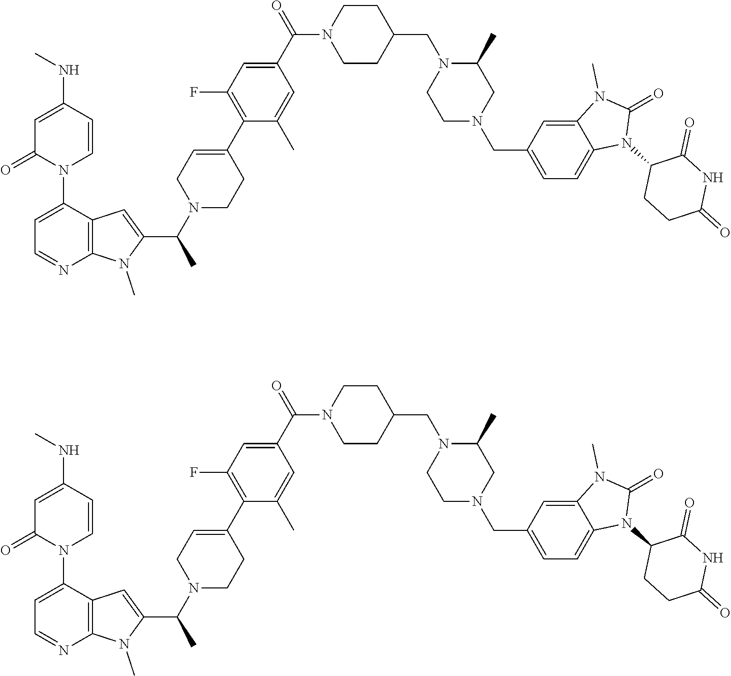 [SMILES: CNc1ccn(-c2ccnc3c2cc([C@H](C)N2CC=C(c4c(C)cc(C(=O)N5CCC(CN6CCN(Cc7ccc8c(c7)n(C)c(=O)n8[C@H]7CCC(=O)NC7=O)C[C@@H]6C)CC5)cc4F)CC2)n3C)c(=O)c1 ; CNc1ccn(-c2ccnc3c2cc([C@H](C)N2CC=C(c4c(C)cc(C(=O)N5CCC(CN6CCN(Cc7ccc8c(c7)n(C)c(=O)n8[C@@H]7CCC(=O)NC7=O)C[C@@H]6C)CC5)cc4F)CC2)n3C)c(=O)c1]

### Example 3u33 and 3u34. (S)-3-(5-(((S)-4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione and (R)-3-(5-(((S)-4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione

Example 3u32 (0.12 g) was resolved by HPLC using a Chiralpak IK 250×30 mm 5 μm column with an eluent of 35% ACN, 35% IPA and 30% DCM (5 mL/min) to give Example 3u33 (25 mg, first peak) and Example 3u34 (40 mg, second peak). The absolute configurations for these compounds at the glutarimide chiral centers were not determined.

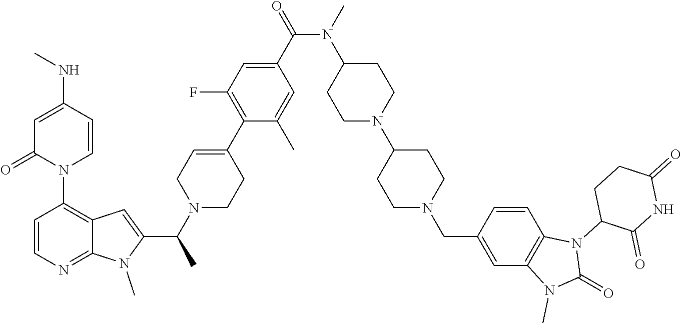 [SMILES: CNc1ccn(-c2ccnc3c2cc([C@H](C)N2CC=C(c4c(C)cc(C(=O)N(C)C5CCN(C6CCN(Cc7ccc8c(c7)n(C)c(=O)n8C7CCC(=O)NC7=O)CC6)CC5)cc4F)CC2)n3C)c(=O)c1]

### Example 3u35. N-(1′-((1-(2,6-dioxopiperidin-3-yl)-3-methyl-2-oxo-2,3-dihydro-1H-benzo[d]imidazol-5-yl)methyl)-[1,4′-bipiperidin]-4-yl)-3-fluoro-N,5-dimethyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzamide

Example 3u35 (9 mg) was prepared similarly to Example 3u163 from Ints. 2G46/3U17 (48 mg/70 mg) using HATU (50 mg) instead of T3P and DIPEA instead of Et₃N.

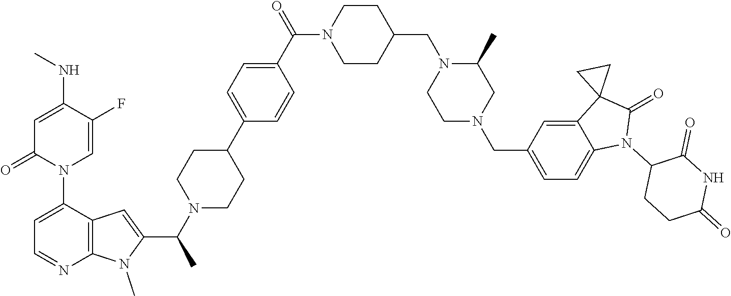 [SMILES: CNc1cc(=O)n(-c2ccnc3c2cc([C@H](C)N2CCC(c4ccc(C(=O)N5CCC(CN6CCN(Cc7ccc8c(c7)C7(CC7)C(=O)N8C7CCC(=O)NC7=O)C[C@@H]6C)CC5)cc4)CC2)n3C)cc1F]

### Example 3u36. 3-(5′-(((S)-4-((1-(4-(1-((S)-1-(4-(5-fluoro-4-(methylamino)-2-oxopyridin-1 (2H)-yl)-1-methyl-1H-pyrrolo[2,3-b]pyridin-2-yl)ethyl)piperidin-4-yl)benzoyl)piperidin-4-yl)methyl)-3-methylpiperazin-1-yl)methyl)-2′-oxospiro[cyclopropane-1,3′-indolin]-1′-yl)piperidine-2,6-dione

Example 3u36 (80 mg) was prepared similarly to Example 3u163 from Ints. 3U91/3C42 (0.20 g/0.31 g).

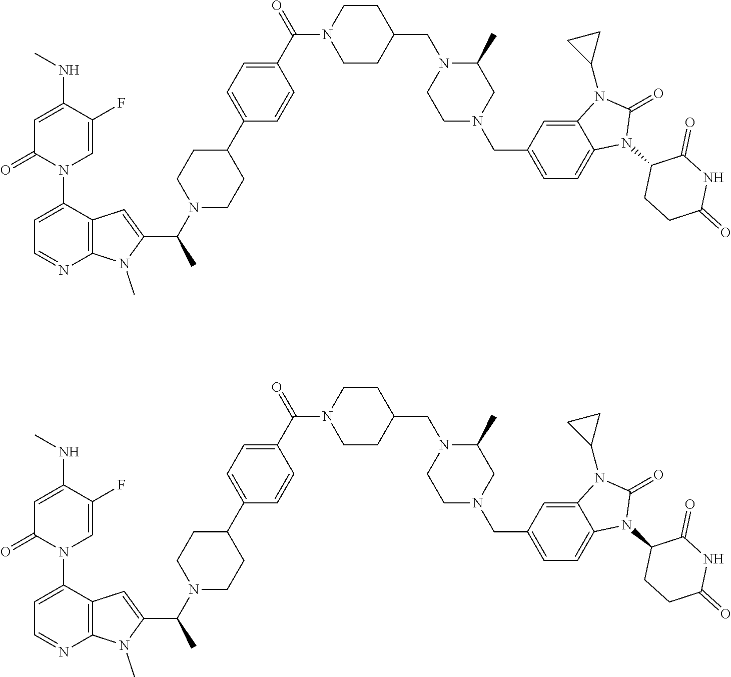 [SMILES: CNc1cc(=O)n(-c2ccnc3c2cc([C@H](C)N2CCC(c4ccc(C(=O)N5CCC(CN6CCN(Cc7ccc8c(c7)n(C7CC7)c(=O)n8[C@H]7CCC(=O)NC7=O)C[C@@H]6C)CC5)cc4)CC2)n3C)cc1F ; CNc1cc(=O)n(-c2ccnc3c2cc([C@H](C)N2CCC(c4ccc(C(=O)N5CCC(CN6CCN(Cc7ccc8c(c7)n(C7CC7)c(=O)n8[C@@H]7CCC(=O)NC7=O)C[C@@H]6C)CC5)cc4)CC2)n3C)cc1F]

### Example 3u37 and 3u38. (S)-3-(5′-(((S)-4-((1-(4-(1-((S)-1-(4-(5-fluoro-4-(methylamino)-2-oxopyridin-1 (2H)-yl)-1-methyl-1H-pyrrolo[2,3-b]pyridin-2-yl)ethyl)piperidin-4-yl)benzoyl)piperidin-4-yl)methyl)-3-methylpiperazin-1-yl)methyl)-2′-oxospiro[cyclopropane-1,3′-indolin]-1′-yl)piperidine-2,6-dione and (R)-3-(5′-(((S)-4-((1-(4-(1-((S)-1-(4-(5-fluoro-4-(methylamino)-2-oxopyridin-1 (2H)-yl)-1-methyl-1H-pyrrolo[2,3-b]pyridin-2-yl)ethyl)piperidin-4-yl)benzoyl)piperidin-4-yl)methyl)-3-methylpiperazin-1-yl)methyl)-2′-oxospiro[cyclopropane-1,3′-indolin]-1′-yl)piperidine-2,6-dione

Example 3u36 (0.12 g) was resolved by HPLC using a Chiralpak IK 250×10 mm 5 μm column with an eluent of 35% ACN, 35% IPA and 30% DCM (4 mL/min) to give Example 3u37 (22 mg, first peak) and Example 3u38 (25 mg, second peak). The absolute configurations for these compounds at the glutarimide chiral centers were not determined.

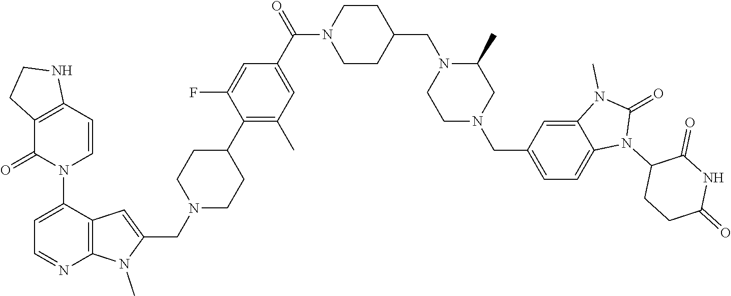 [SMILES: Cc1cc(C(=O)N2CCC(CN3CCN(Cc4ccc5c(c4)n(C)c(=O)n5C4CCC(=O)NC4=O)C[C@@H]3C)CC2)cc(F)c1C1CCN(Cc2cc3c(-n4ccc5c(c4=O)CCN5)ccnc3n2C)CC1]

### Example 3u39. 3-(5-(((S)-4-((1-(3-fluoro-5-methyl-4-(1-((1-methyl-4-(4-oxo-1,2,3,4-tetrahydro-5H-pyrrolo[3,2-c]pyridin-5-yl)-1H-pyrrolo[2,3-b]pyridin-2-yl)methyl)piperidin-4-yl)benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione

Example 3u39 (0.13 g) was prepared similarly to Example 3u163 from Ints. 2G56/3E68 (0.40 g/0.42 g).

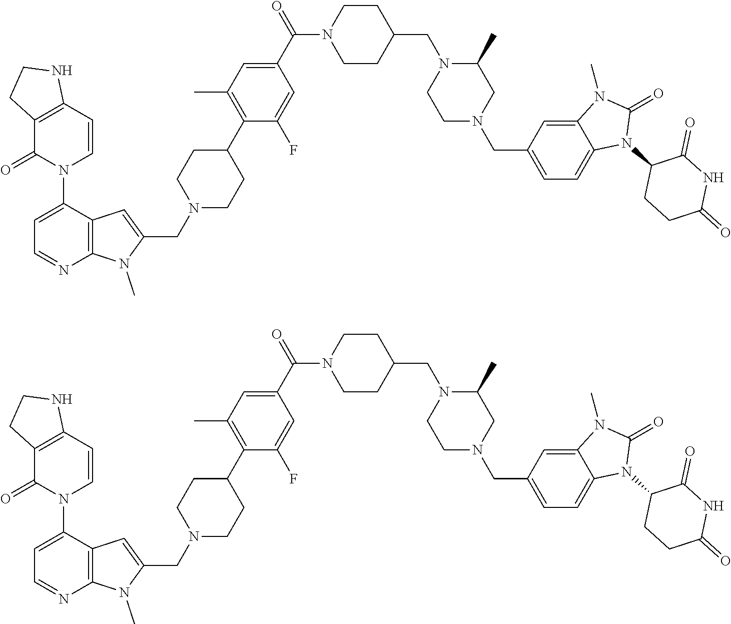 [SMILES: Cc1cc(C(=O)N2CCC(CN3CCN(Cc4ccc5c(c4)n(C)c(=O)n5[C@@H]4CCC(=O)NC4=O)C[C@@H]3C)CC2)cc(F)c1C1CCN(Cc2cc3c(-n4ccc5c(c4=O)CCN5)ccnc3n2C)CC1 ; Cc1cc(C(=O)N2CCC(CN3CCN(Cc4ccc5c(c4)n(C)c(=O)n5[C@H]4CCC(=O)NC4=O)C[C@@H]3C)CC2)cc(F)c1C1CCN(Cc2cc3c(-n4ccc5c(c4=O)CCN5)ccnc3n2C)CC1]

### Example 3u40 and 3u41. (R)-3-(5-(((S)-4-((1-(3-fluoro-5-methyl-4-(1-((1-methyl-4-(4-oxo-1,2,3,4-tetrahydro-5H-pyrrolo[3,2-c]pyridin-5-yl)-1H-pyrrolo[2,3-b]pyridin-2-yl)methyl)piperidin-4-yl)benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione and (S)-3-(5-(((S)-4-((1-(3-fluoro-5-methyl-4-(1-((1-methyl-4-(4-oxo-1,2,3,4-tetrahydro-5H-pyrrolo[3,2-c]pyridin-5-yl)-1H-pyrrolo[2,3-b]pyridin-2-yl)methyl)piperidin-4-yl)benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione

Example 3u39 (0.13 g) was resolved by HPLC using a Chiralpak IK 250×10 mm 5 μm column with an eluent of 35% ACN, 35% IPA and 30% DCM (4 mL/min) to give Example 3u40 (17 mg, first peak) and Example 3u41 (17 mg, second peak). The absolute configurations for these compounds at the glutarimide chiral centers were not determined.

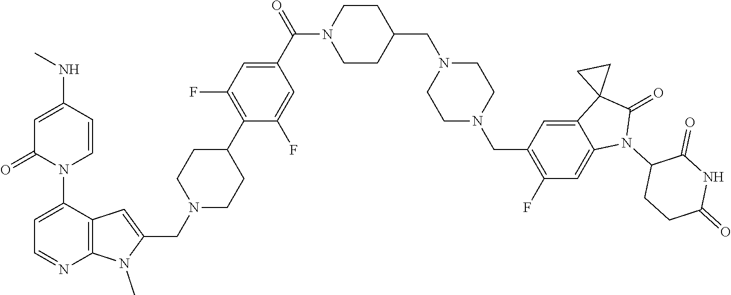 [SMILES: CNc1ccn(-c2ccnc3c2cc(CN2CCC(c4c(F)cc(C(=O)N5CCC(CN6CCN(Cc7cc8c(cc7F)N(C7CCC(=O)NC7=O)C(=O)C87CC7)CC6)CC5)cc4F)CC2)n3C)c(=O)c1]

### Example 3u42. 3-(5′-((4-((1-(3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)methyl)piperazin-1-yl)methyl)-6′-fluoro-2′-oxospiro[cyclopropane-1,3′-indolin]-1′-yl)piperidine-2,6-dione

Example 3u42 (80 mg) was prepared similarly to Example 3u163 from Ints. 2G108/3C4 (0.25 g/0.44 g).

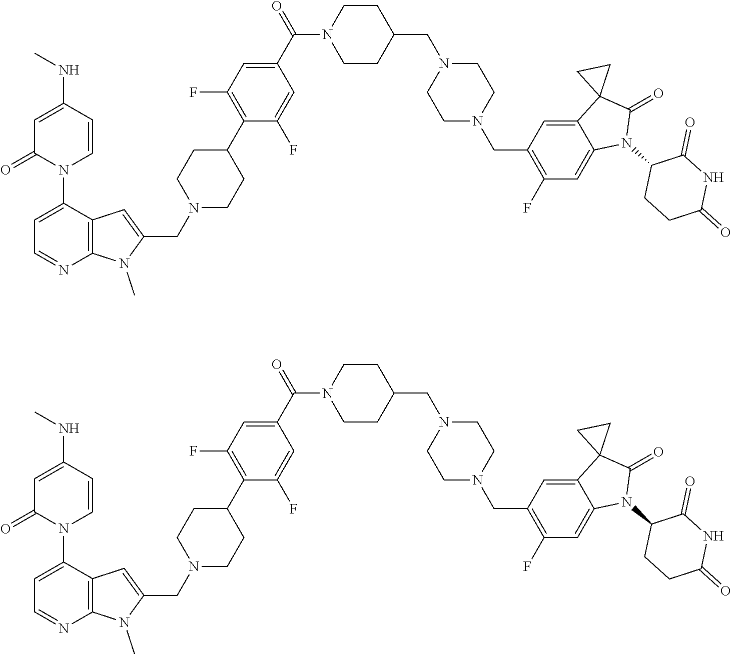 [SMILES: CNc1ccn(-c2ccnc3c2cc(CN2CCC(c4c(F)cc(C(=O)N5CCC(CN6CCN(Cc7cc8c(cc7F)N([C@H]7CCC(=O)NC7=O)C(=O)C87CC7)CC6)CC5)cc4F)CC2)n3C)c(=O)c1 ; CNc1ccn(-c2ccnc3c2cc(CN2CCC(c4c(F)cc(C(=O)N5CCC(CN6CCN(Cc7cc8c(cc7F)N([C@@H]7CCC(=O)NC7=O)C(=O)C87CC7)CC6)CC5)cc4F)CC2)n3C)c(=O)c1]

### Example 3u43 and 3u44. (S)-3-(5′-((4-((1-(3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)methyl)piperazin-1-yl)methyl)-6′-fluoro-2′-oxospiro[cyclopropane-1,3′-indolin]-1′-yl)piperidine-2,6-dione and (R)-3-(5′-((4-((1-(3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)methyl)piperazin-1-yl)methyl)-6′-fluoro-2′-oxospiro[cyclopropane-1,3′-indolin]-1′-yl)piperidine-2,6-dione

Example 3u42 (80 mg) was resolved by HPLC using a CELLULOSE-SC 250×30 mm 5 μm column with an eluent of 35% ACN, 35% IPA and 30% DCM (42 mL/min) to give Example 3u43 (15 mg, first peak) and Example 3u44 (6 mg, second peak). The absolute configurations for these compounds at the glutarimide chiral centers were not determined.

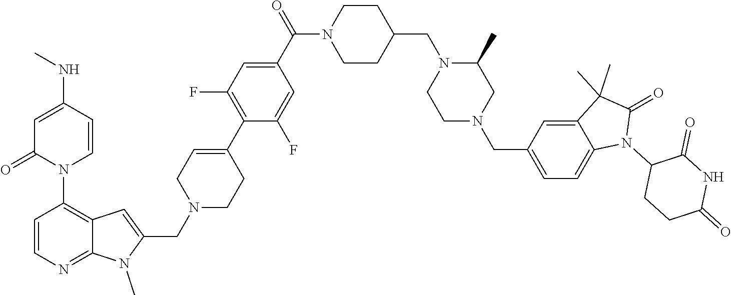 [SMILES: CNc1ccn(-c2ccnc3c2cc(CN2CC=C(c4c(F)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)c(=O)c1]

### Example 3u45. 3-(5-(((S)-4-((1-(3,5-difluoro-4-(1-((1-methyl-4-(4-(methylamino)-2-oxopyridin-1 (2H)-yl)-1H-pyrrolo[2,3-b]pyridin-2-yl)methyl)-1,2,3,6-tetrahydropyridin-4-yl)benzoyl)piperidin-4-yl)methyl)-3-methylpiperazin-1-yl)methyl)-3,3-dimethyl-2-oxoindolin-1-yl)piperidine-2,6-dione

Example 3u45 (0.16 g) was prepared similarly to Example 3u163 from Ints. 3U90/3E291 (0.30 g/0.34 g).

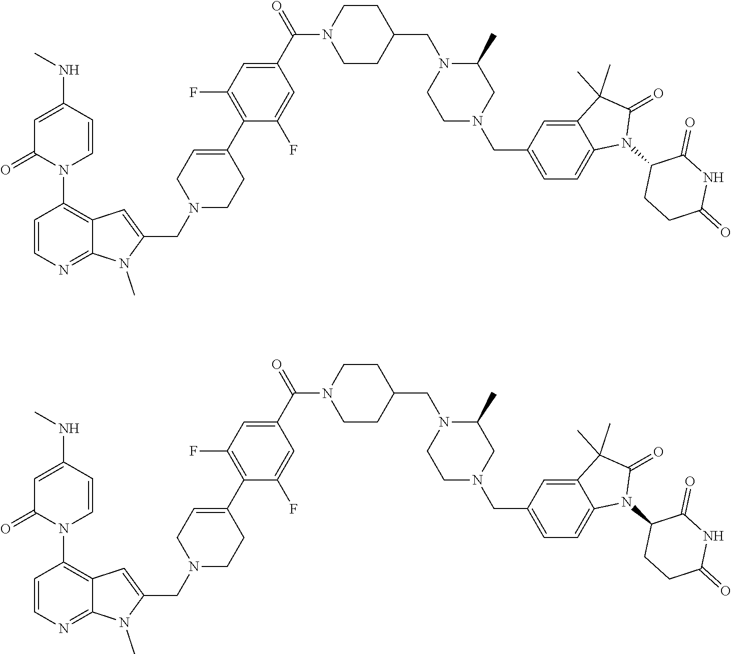 [SMILES: CNc1ccn(-c2ccnc3c2cc(CN2CC=C(c4c(F)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)c(=O)c1 ; CNc1ccn(-c2ccnc3c2cc(CN2CC=C(c4c(F)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)c(=O)c1]

### Example 3u46 and 3u47. (S)-3-(5-(((S)-4-((1-(3,5-difluoro-4-(1-((1-methyl-4-(4-(methylamino)-2-oxopyridin-1 (2H)-yl)-1H-pyrrolo[2,3-b]pyridin-2-yl)methyl)-1,2,3,6-tetrahydropyridin-4-yl)benzoyl)piperidin-4-yl)methyl)-3-methylpiperazin-1-yl)methyl)-3,3-dimethyl-2-oxoindolin-1-yl)piperidine-2,6-dione and (R)-3-(5-(((S)-4-((1-(3,5-difluoro-4-(1-((1-methyl-4-(4-(methylamino)-2-oxopyridin-1 (2H)-yl)-1H-pyrrolo[2,3-b]pyridin-2-yl)methyl)-1,2,3,6-tetrahydropyridin-4-yl)benzoyl)piperidin-4-yl)methyl)-3-methylpiperazin-1-yl)methyl)-3,3-dimethyl-2-oxoindolin-1-yl)piperidine-2,6-dione

Example 3u45 (0.14 g) was resolved by HPLC using a Chiralpak IK 250×30 mm 5 μm column with an eluent of 35% ACN, 35% IPA and 30% DCM (37 mL/min) to give Example 3u46 (37 mg, first peak) and Example 3u47 (39 mg, second peak). The absolute configurations for these compounds at the glutarimide chiral centers were not determined.

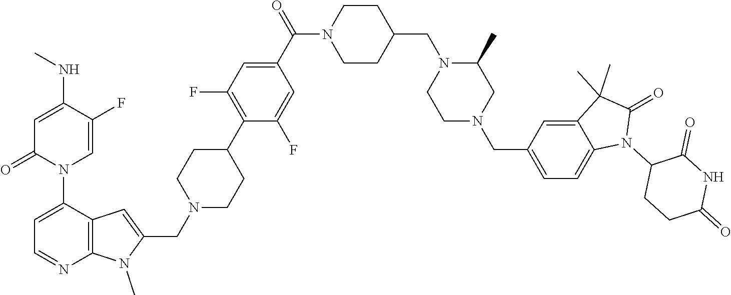 [SMILES: CNc1cc(=O)n(-c2ccnc3c2cc(CN2CCC(c4c(F)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)cc1F]

### Example 3u48. 3-(5-(((S)-4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)methyl)-3-methylpiperazin-1-yl)methyl)-3,3-dimethyl-2-oxoindolin-1-yl)piperidine-2,6-dione

Example 3u48 (0.10 g) was prepared similarly to Example 3u163 from Ints. 2G58/3E291 (0.30 g/0.34 g).

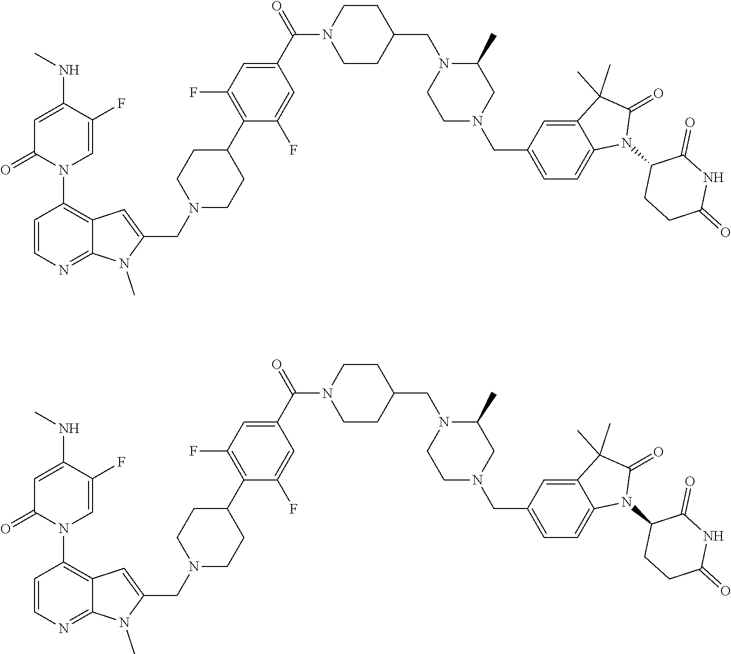 [SMILES: CNc1cc(=O)n(-c2ccnc3c2cc(CN2CCC(c4c(F)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)cc1F ; CNc1cc(=O)n(-c2ccnc3c2cc(CN2CCC(c4c(F)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)cc1F]

### Example 3u49 and 3u50. (S)-3-(5-(((S)-4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)methyl)-3-methylpiperazin-1-yl)methyl)-3,3-dimethyl-2-oxoindolin-1-yl)piperidine-2,6-dione and (R)-3-(5-(((S)-4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)methyl)-3-methylpiperazin-1-yl)methyl)-3,3-dimethyl-2-oxoindolin-1-yl)piperidine-2,6-dione

Example 3u48 (0.10 g) was resolved by HPLC using a Chiralpak IK 250×30 mm 5 μm column with an eluent of 35% ACN, 35% IPA and 30% DCM (38 mL/min) to give Example 3u49 (10 mg, first peak) and Example 3u50 (8 mg, second peak). The absolute configurations for these compounds at the glutarimide chiral centers were not determined.

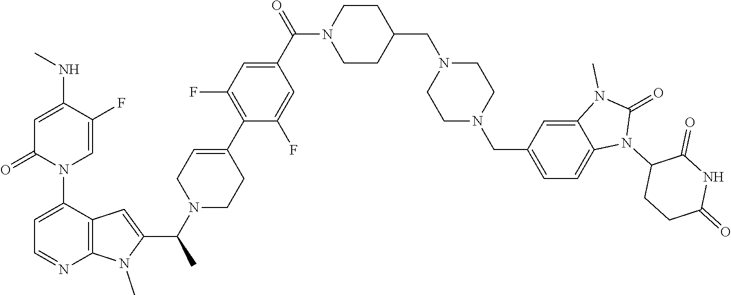 [SMILES: CNc1cc(=O)n(-c2ccnc3c2cc([C@H](C)N2CC=C(c4c(F)cc(C(=O)N5CCC(CN6CCN(Cc7ccc8c(c7)n(C)c(=O)n8C7CCC(=O)NC7=O)CC6)CC5)cc4F)CC2)n3C)cc1F]

### Example 3u51. 3-(5-((4-((1-(3,5-difluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)benzoyl)piperidin-4-yl)methyl)piperazin-1-yl)methyl)-3-methyl-2-oxo-2,3-dihydro-1H-benzo[d]imidazol-1-yl)piperidine-2,6-dione

Example 3u51 (85 mg) was prepared similarly to Example 3u163 from Ints. 2G43/3E1 (0.20 g/0.24 g).

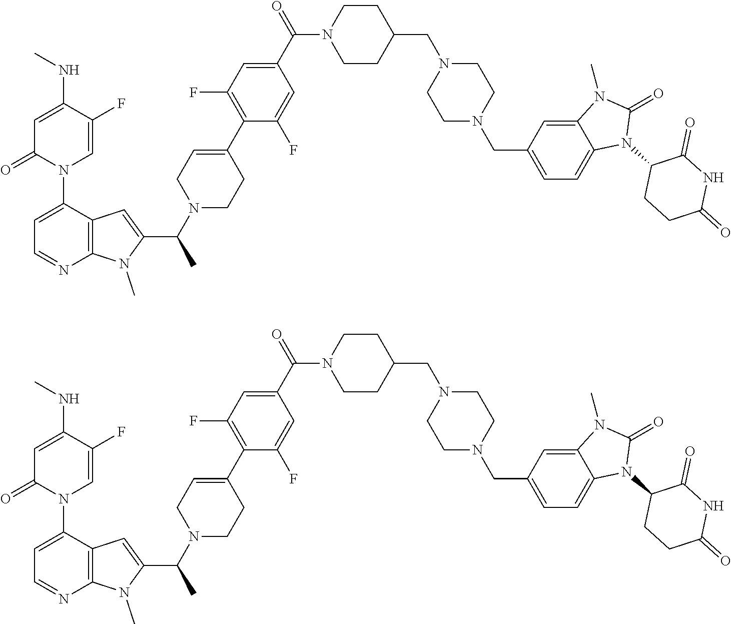 [SMILES: CNc1cc(=O)n(-c2ccnc3c2cc([C@H](C)N2CC=C(c4c(F)cc(C(=O)N5CCC(CN6CCN(Cc7ccc8c(c7)n(C)c(=O)n8[C@H]7CCC(=O)NC7=O)CC6)CC5)cc4F)CC2)n3C)cc1F ; CNc1cc(=O)n(-c2ccnc3c2cc([C@H](C)N2CC=C(c4c(F)cc(C(=O)N5CCC(CN6CCN(Cc7ccc8c(c7)n(C)c(=O)n8[C@@H]7CCC(=O)NC7=O)CC6)CC5)cc4F)CC2)n3C)cc1F]

### Example 3u52 and 3u53. (S)-3-(5-((4-((1-(3,5-difluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)benzoyl)piperidin-4-yl)methyl)piperazin-1-yl)methyl)-3-methyl-2-oxo-2,3-dihydro-1H-benzo[d]imidazol-1-yl)piperidine-2,6-dione and (R)-3-(5-((4-((1-(3,5-difluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)benzoyl)piperidin-4-yl)methyl)piperazin-1-yl)methyl)-3-methyl-2-oxo-2,3-dihydro-1H-benzo[d]imidazol-1-yl)piperidine-2,6-dione

Example 3u51 (75 mg) was resolved by HPLC using a Chiralpak IK 250×10 mm 5 μm column with an eluent of 35% ACN, 35% IPA and 30% DCM (5 mL/min) to give Example 3u52 (10 mg, first peak) and Example 3u53 (24 mg, second peak). The absolute configurations for these compounds at the glutarimide chiral centers were not determined.

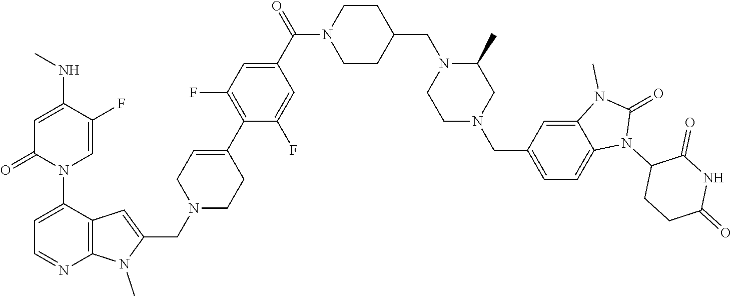 [SMILES: CNc1cc(=O)n(-c2ccnc3c2cc(CN2CC=C(c4c(F)cc(C(=O)N5CCC(CN6CCN(Cc7ccc8c(c7)n(C)c(=O)n8C7CCC(=O)NC7=O)C[C@@H]6C)CC5)cc4F)CC2)n3C)cc1F]

### Example 3u54. 3-(5-(((S)-4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)-1,2,3,6-tetrahydropyridin-4-yl)benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione

Example 3u54 (0.18 g) was prepared similarly to Example 3u163 from Ints. 2G48/3E68 (0.25 g/0.36 g).

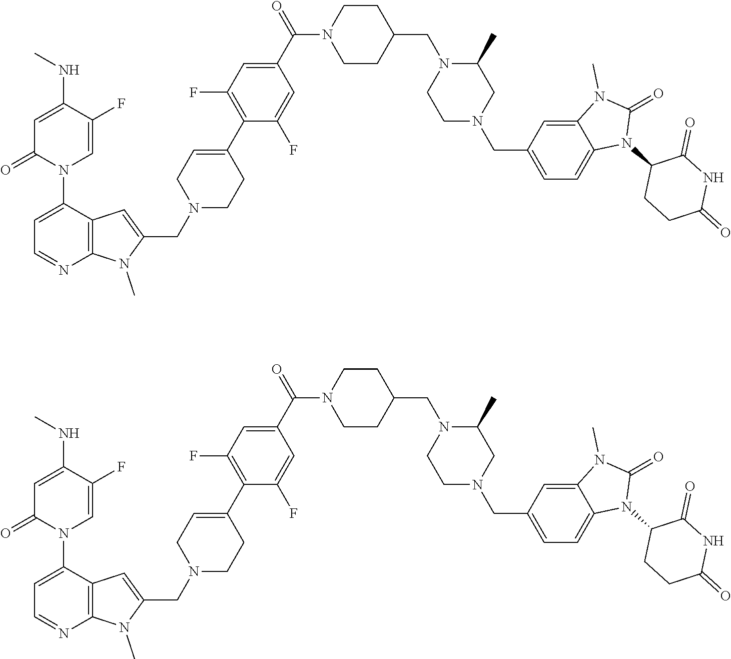 [SMILES: CNc1cc(=O)n(-c2ccnc3c2cc(CN2CC=C(c4c(F)cc(C(=O)N5CCC(CN6CCN(Cc7ccc8c(c7)n(C)c(=O)n8[C@@H]7CCC(=O)NC7=O)C[C@@H]6C)CC5)cc4F)CC2)n3C)cc1F ; CNc1cc(=O)n(-c2ccnc3c2cc(CN2CC=C(c4c(F)cc(C(=O)N5CCC(CN6CCN(Cc7ccc8c(c7)n(C)c(=O)n8[C@H]7CCC(=O)NC7=O)C[C@@H]6C)CC5)cc4F)CC2)n3C)cc1F]

### Example 3u55 and 3u56. (R)-3-(5-(((S)-4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)-1,2,3,6-tetrahydropyridin-4-yl)benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione and (S)-3-(5-(((S)-4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)-1,2,3,6-tetrahydropyridin-4-yl)benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione

Example 3u54 (0.11 g) was resolved by HPLC using a Chiralpak IK 250×30 mm 5 μm column with an eluent of 35% ACN, 35% IPA and 30% DCM (40 mL/min) to give Example 3u55 (46 mg, first peak) and Example 3u56 (14 mg, second peak). The second peak required a subsequent HPLC-purification. The absolute configurations for these compounds at the glutarimide chiral centers were not determined.

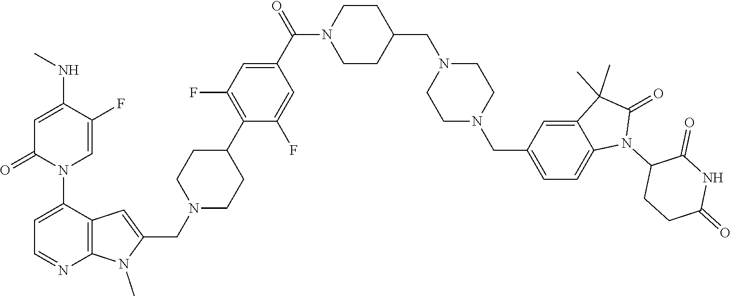 [SMILES: CNc1cc(=O)n(-c2ccnc3c2cc(CN2CCC(c4c(F)cc(C(=O)N5CCC(CN6CCN(Cc7ccc8c(c7)C(C)(C)C(=O)N8C7CCC(=O)NC7=O)CC6)CC5)cc4F)CC2)n3C)cc1F]

### Example 3u57. 3-(5-((4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)methyl)piperazin-1-yl)methyl)-3,3-dimethyl-2-oxoindolin-1-yl)piperidine-2,6-dione

Example 3u57 (94 mg) was prepared similarly to Example 3u163 from Ints. 2G58/3U7 (0.30 g/0.46 g).

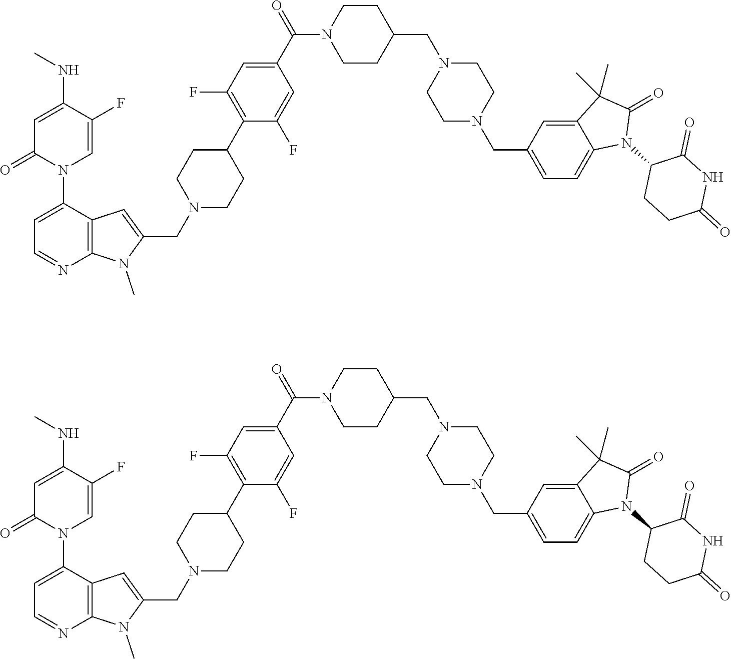 [SMILES: CNc1cc(=O)n(-c2ccnc3c2cc(CN2CCC(c4c(F)cc(C(=O)N5CCC(CN6CCN(Cc7ccc8c(c7)C(C)(C)C(=O)N8[C@H]7CCC(=O)NC7=O)CC6)CC5)cc4F)CC2)n3C)cc1F ; CNc1cc(=O)n(-c2ccnc3c2cc(CN2CCC(c4c(F)cc(C(=O)N5CCC(CN6CCN(Cc7ccc8c(c7)C(C)(C)C(=O)N8[C@@H]7CCC(=O)NC7=O)CC6)CC5)cc4F)CC2)n3C)cc1F]

### Example 3u58 and 3u59. (S)-3-(5-((4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)methyl)piperazin-1-yl)methyl)-3,3-dimethyl-2-oxoindolin-1-yl)piperidine-2,6-dione and (R)-3-(5-((4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)methyl)piperazin-1-yl)methyl)-3,3-dimethyl-2-oxoindolin-1-yl)piperidine-2,6-dione

Example 3u57 (70 mg) was resolved by HPLC using a Chiralpak IK 250×30 mm 5 μm column with an eluent of 35% ACN, 35% IPA and 30% DCM (3 mL/min) to give Example 3u58 (17 mg, first peak) and Example 3u59 (27 mg, second peak). The absolute configurations for these compounds were not determined.

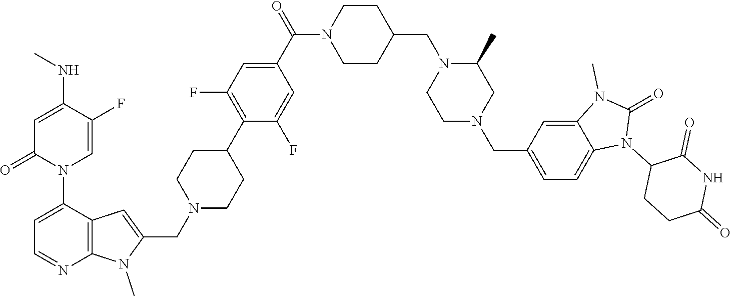 [SMILES: CNc1cc(=O)n(-c2ccnc3c2cc(CN2CCC(c4c(F)cc(C(=O)N5CCC(CN6CCN(Cc7ccc8c(c7)n(C)c(=O)n8C7CCC(=O)NC7=O)C[C@@H]6C)CC5)cc4F)CC2)n3C)cc1F]

### Example 3u60. 3-(5-(((S)-4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione

Example 3u60 (95 mg) was prepared similarly to Example 3u163 from Ints. 2G58/3E68 (0.25 g/0.29 g).

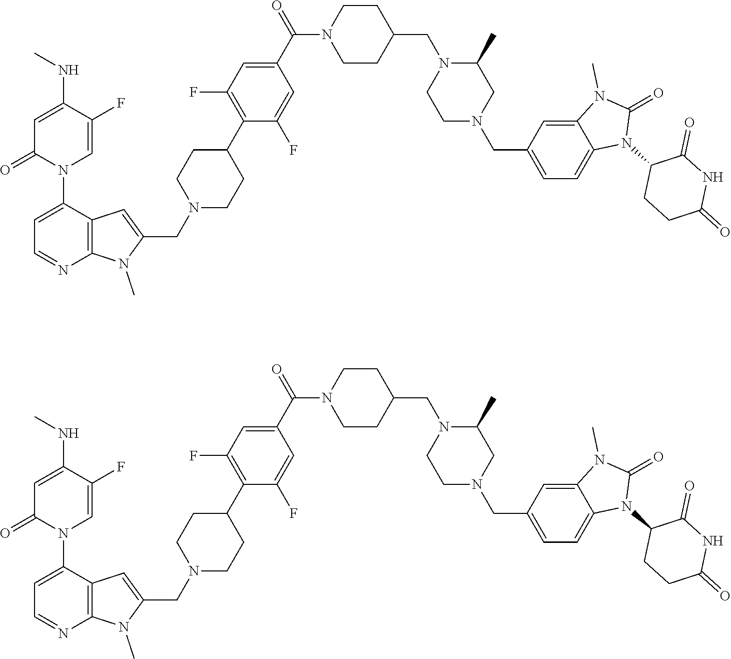 [SMILES: CNc1cc(=O)n(-c2ccnc3c2cc(CN2CCC(c4c(F)cc(C(=O)N5CCC(CN6CCN(Cc7ccc8c(c7)n(C)c(=O)n8[C@H]7CCC(=O)NC7=O)C[C@@H]6C)CC5)cc4F)CC2)n3C)cc1F ; CNc1cc(=O)n(-c2ccnc3c2cc(CN2CCC(c4c(F)cc(C(=O)N5CCC(CN6CCN(Cc7ccc8c(c7)n(C)c(=O)n8[C@@H]7CCC(=O)NC7=O)C[C@@H]6C)CC5)cc4F)CC2)n3C)cc1F]

### Example 3u61 and 3u62. (S)-3-(5-(((S)-4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione and (R)-3-(5-(((S)-4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione

Example 3u60 (90 mg) was resolved by HPLC using a CELLULOSE-SC 250×30 mm 5 μm column with an eluent of 35% ACN, 35% IPA and 30% DCM (42 mL/min) to give Example 3u62 (20 mg, first peak) and Example 3u61 (21 mg, second peak). The absolute configurations for these compounds at the glutarimide chiral centers were not determined.

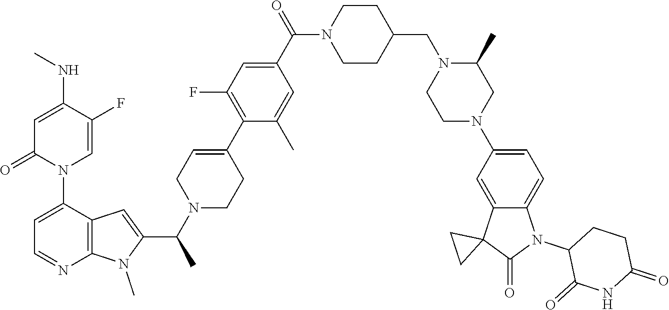 [SMILES: CNc1cc(=O)n(-c2ccnc3c2cc([C@H](C)N2CC=C(c4c(C)cc(C(=O)N5CCC(CN6CCN(c7ccc8c(c7)C7(CC7)C(=O)N8C7CCC(=O)NC7=O)C[C@@H]6C)CC5)cc4F)CC2)n3C)cc1F]

### Example 3u63. 3-(5′-((S)-4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)-piperidin-4-yl)methyl)-3-methylpiperazin-1-yl)-2′-oxospiro[cyclopropane-1,3′-indolin]-1′-yl)-piperidine-2,6-dione

Example 3u63 (0.12 g) was prepared similarly to Example 3u163 from Ints. 2G45/3C13 (0.30 g/0.55 g).

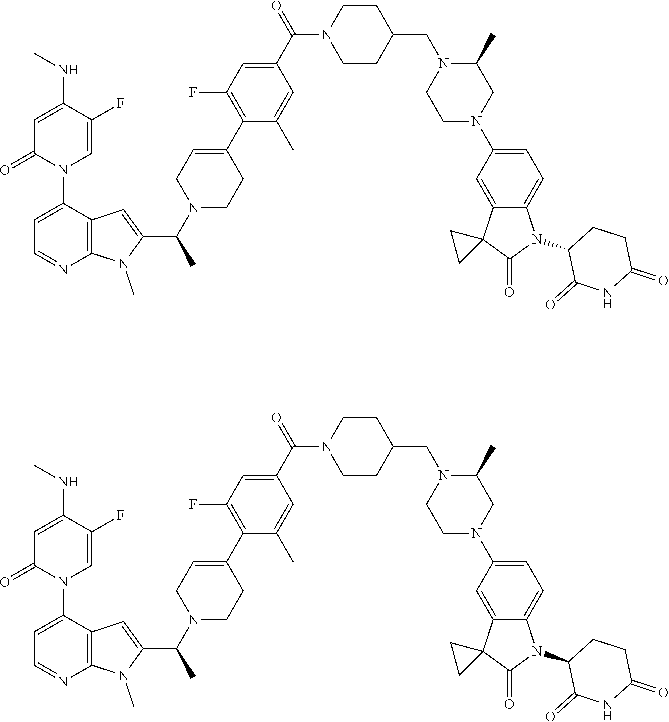 [SMILES: CNc1cc(=O)n(-c2ccnc3c2cc([C@H](C)N2CC=C(c4c(C)cc(C(=O)N5CCC(CN6CCN(c7ccc8c(c7)C7(CC7)C(=O)N8[C@@H]7CCC(=O)NC7=O)C[C@@H]6C)CC5)cc4F)CC2)n3C)cc1F ; CNc1cc(=O)n(-c2ccnc3c2cc([C@H](C)N2CC=C(c4c(C)cc(C(=O)N5CCC(CN6CCN(c7ccc8c(c7)C7(CC7)C(=O)N8[C@H]7CCC(=O)NC7=O)C[C@@H]6C)CC5)cc4F)CC2)n3C)cc1F]

### Example 3u64 and 3u65. (R)-3-(5′-((S)-4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)-3-methylpiperazin-1-yl)-2′-oxospiro[cyclopropane-1,3′-indolin]-1′-yl)piperidine-2,6-dione and (S)-3-(5′-((S)-4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)-3-methylpiperazin-1-yl)-2′-oxospiro[cyclopropane-1,3′-indolin]-1′-yl)piperidine-2,6-dione

Example 3u63 (0.10 g) was resolved by HPLC using a CELLULOSE-SZ 250×30 mm 5 μm column with an eluent of 35% ACN, 35% IPA and 30% DCM (22 mL/min) to give Example 3u64 (26 mg, first peak) and Example 3u65 (17 mg, second peak). The absolute configurations for these compounds at the glutarimide chiral centers were not determined.

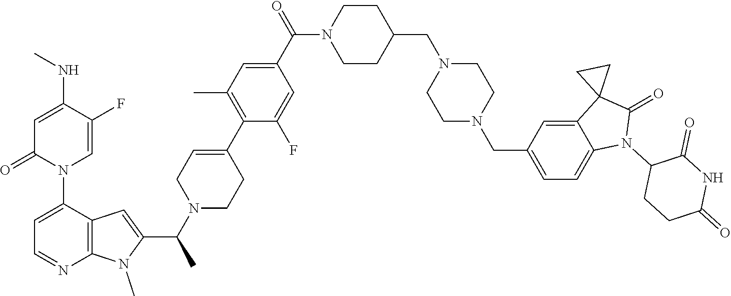 [SMILES: CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)CC6)CC5)cc4F)CC2)n3C)cc1F]

### Example 3u66. 3-(5′-((4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)-piperidin-4-yl)methyl)piperazin-1-yl)methyl)-2′-oxospiro[cyclopropane-1,3′-indolin]-1′-yl)piperidine-2,6-dione

Example 3u66 (85 mg) was prepared similarly to Example 3u163 from Ints. 2G45/3U21 (0.20 g/0.37 g).

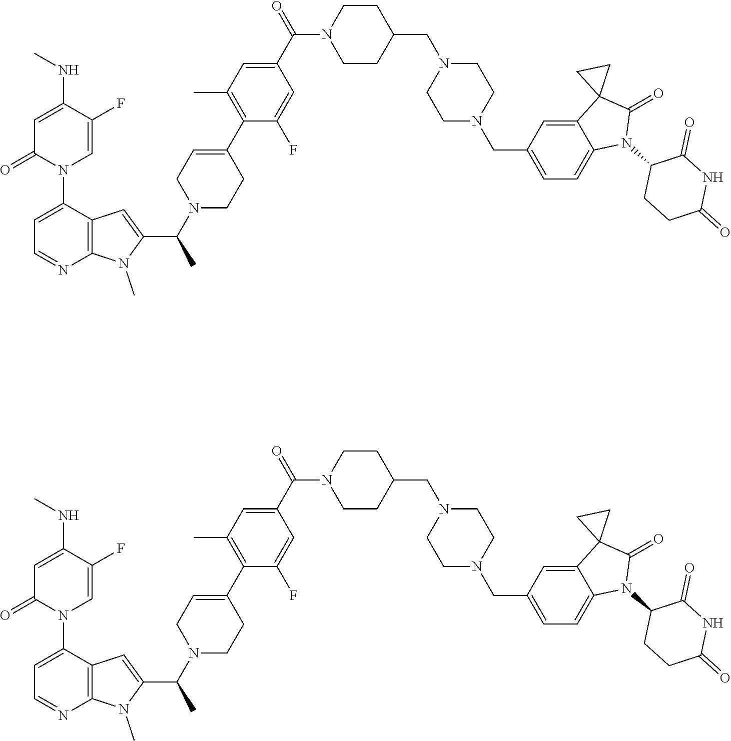 [SMILES: CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8[C@H]7CCC(=O)NC7=O)CC6)CC5)cc4F)CC2)n3C)cc1F ; CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8[C@@H]7CCC(=O)NC7=O)CC6)CC5)cc4F)CC2)n3C)cc1F]

### Example 3u67 and 3u68. (S)-3-(5′-((4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)methyl)-2′-oxospiro[cyclopropane-1,3′-indolin]-1′-yl)piperidine-2,6-dione and (R)-3-(5′-((4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)methyl)-2′-oxospiro[cyclopropane-1,3′-indolin]-1′-yl)piperidine-2,6-dione

Example 3u66 (65 mg) was resolved by HPLC using a Chiralpak IK 250×10 mm 5 μm column with an eluent of 35% ACN, 35% IPA and 30% DCM (3 mL/min) to give Example 3u67 (10 mg, first peak) and Example 3u68 (12 mg, second peak). The absolute configurations for these compounds at the glutarimide chiral centers were not determined.

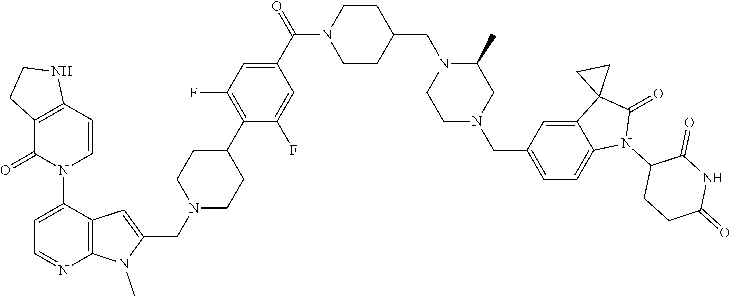 [SMILES: C[C@H]1CN(Cc2ccc3c(c2)C2(CC2)C(=O)N3C2CCC(=O)NC2=O)CCN1CC1CCN(C(=O)c2cc(F)c(C3CCN(Cc4cc5c(-n6ccc7c(c6=O)CCN7)ccnc5n4C)CC3)c(F)c2)CC1]

### Example 3u69. 3-(5′-(((S)-4-((1-(3,5-difluoro-4-(1-((1-methyl-4-(4-oxo-1,2,3,4-tetrahydro-5H-pyrrolo[3,2-c]pyridin-5-yl)-1H-pyrrolo[2,3-b]pyridin-2-yl)methyl)piperidin-4-yl)benzoyl)piperidin-4-yl)methyl)-3-methylpiperazin-1-yl)methyl)-2′-oxospiro[cyclopropane-1,3′-indolin]-1′-yl)piperidine-2,6-dione

Example 3u69 (80 mg) was prepared similarly to Example 3u163 from Ints. 2G57/3C42 (0.30 g/0.37 g).

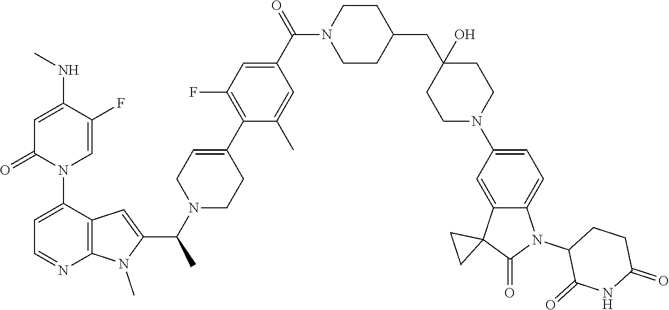 [SMILES: CNc1cc(=O)n(-c2ccnc3c2cc([C@H](C)N2CC=C(c4c(C)cc(C(=O)N5CCC(CC6(O)CCN(c7ccc8c(c7)C7(CC7)C(=O)N8C7CCC(=O)NC7=O)CC6)CC5)cc4F)CC2)n3C)cc1F]

### Example 3u70. 3-(5′-(4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)-piperidin-4-yl)methyl)-4-hydroxypiperidin-1-yl)-2′-oxospiro[cyclopropane-1,3′-indolin]-1′-yl)-piperidine-2,6-dione

Example 3u70 (15 mg) was prepared similarly to Example 3u163 from Ints. 2G45/3U23 (45 mg/0.14 g).

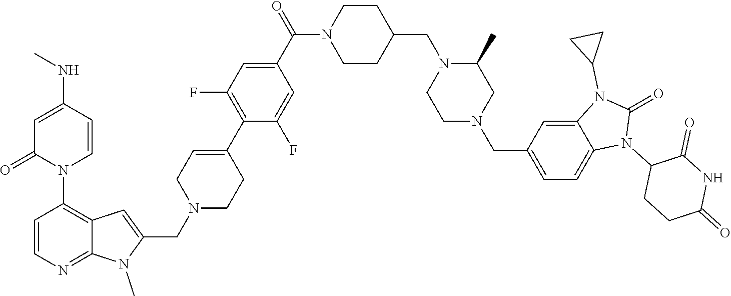 [SMILES: CNc1ccn(-c2ccnc3c2cc(CN2CC=C(c4c(F)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)c(=O)c1]

### Example 3u71. 3-(3-cyclopropyl-5-(((S)-4-((1-(3,5-difluoro-4-(1-((1-methyl-4-(4-(methylamino)-2-oxopyridin-1 (2H)-yl)-1H-pyrrolo[2,3-b]pyridin-2-yl)methyl)-1,2,3,6-tetrahydropyridin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione

Example 3u71 (0.16 g) was prepared similarly to Example 3u163 from Ints. 3U88/3E78 (0.30 g/0.35 g).

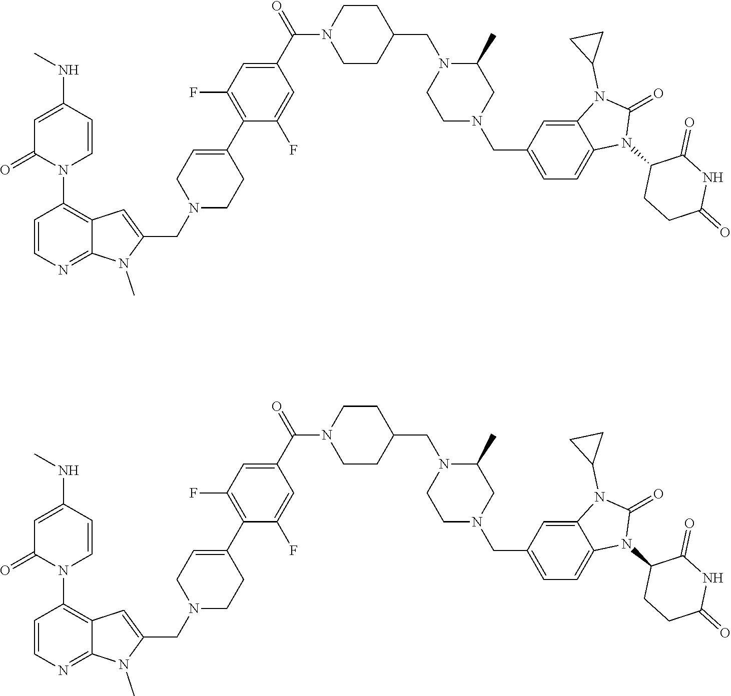 [SMILES: CNc1ccn(-c2ccnc3c2cc(CN2CC=C(c4c(F)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)c(=O)c1 ; CNc1ccn(-c2ccnc3c2cc(CN2CC=C(c4c(F)cc(C(=O)N5CCC(CN6CCN(Cc7ccc8c(c7)n(C7CC7)c(=O)n8[C@@H]7CCC(=O)NC7=O)C[C@@H]6C)CC5)cc4F)CC2)n3C)c(=O)c1]

### Example 3u72 and 3u73. (S)-3-(3-cyclopropyl-5-(((S)-4-((1-(3,5-difluoro-4-(1-((1-methyl-4-(4-(methylamino)-2-oxopyridin-1 (2H)-yl)-1H-pyrrolo[2,3-b]pyridin-2-yl)methyl)-1,2,3,6-tetrahydropyridin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione and (R)-3-(3-cyclopropyl-5-(((S)-4-((1-(3,5-difluoro-4-(1-((1-methyl-4-(4-(methylamino)-2-oxopyridin-1 (2H)-yl)-1H-pyrrolo[2,3-b]pyridin-2-yl)methyl)-1,2,3,6-tetrahydropyridin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione

Example 3u71 (0.14 g) was resolved by HPLC using a Chiralpak IK 250×30 mm 5 μm column with an eluent of 35% ACN, 35% IPA and 30% DCM (40 mL/min) to give Example 3u72 (35 mg, first peak) and Example 3u73 (60 mg, second peak). The absolute configurations for these compounds at the glutarimide chiral centers were not determined.

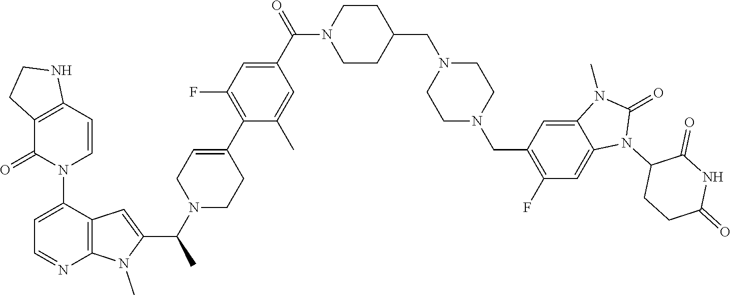 [SMILES: Cc1cc(C(=O)N2CCC(CN3CCN(Cc4cc5c(cc4F)n(C4CCC(=O)NC4=O)c(=O)n5C)CC3)CC2)cc(F)c1C1=CCN([C@@H](C)c2cc3c(-n4ccc5c(c4=O)CCN5)ccnc3n2C)CC1]

### Example 3u74. 3-(6-fluoro-5-((4-((1-(3-fluoro-5-methyl-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydro-pyridin-4-yl)benzoyl)piperidin-4-yl)methyl)piperazin-1-yl)methyl)-3-methyl-2-oxo-2,3-dihydro-1H-benzo[d]imidazol-1-yl)piperidine-2,6-dione

Example 3u74 (0.14 g) was prepared similarly to Example 3u163 from Ints. 2G53/3E145 (0.15 g/0.20 g).

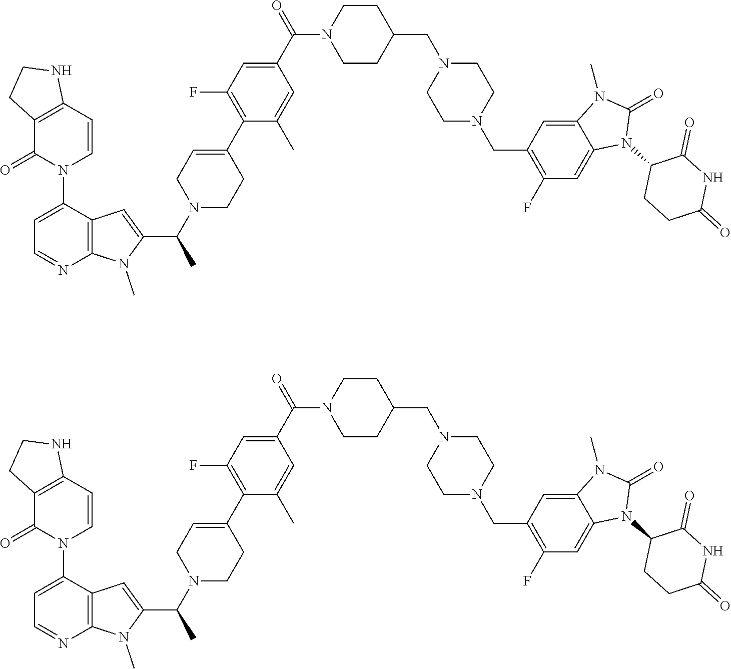 [SMILES: Cc1cc(C(=O)N2CCC(CN3CCN(Cc4cc5c(cc4F)n([C@H]4CCC(=O)NC4=O)c(=O)n5C)CC3)CC2)cc(F)c1C1=CCN([C@@H](C)c2cc3c(-n4ccc5c(c4=O)CCN5)ccnc3n2C)CC1 ; Cc1cc(C(=O)N2CCC(CN3CCN(Cc4cc5c(cc4F)n([C@@H]4CCC(=O)NC4=O)c(=O)n5C)CC3)CC2)cc(F)c1C1=CCN([C@@H](C)c2cc3c(-n4ccc5c(c4=O)CCN5)ccnc3n2C)CC1]

### Example 3u75 and 3u76. (S)-3-(6-fluoro-5-((4-((1-(3-fluoro-5-methyl-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)methyl)-3-methyl-2-oxo-2,3-dihydro-1H-benzo[d]imidazol-1-yl)piperidine-2,6-dione and (R)-3-(6-fluoro-5-((4-((1-(3-fluoro-5-methyl-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzoyl)piperidin-4-yl)methyl)-piperazin-1-yl)methyl)-3-methyl-2-oxo-2,3-dihydro-1H-benzo[d]imidazol-1-yl)piperidine-2,6-dione

Example 3u74 (0.10 g) was resolved by HPLC using a Chiralpak IK 250×10 mm 5 μm column with an eluent of 40% ACN, 40% IPA and 20% DCM (4 mL/min) to give Example 3u75 (28 mg, first peak) and Example 2u76 (39 mg, second peak). The absolute configurations for these compounds at the glutarimide chiral centers were not determined.

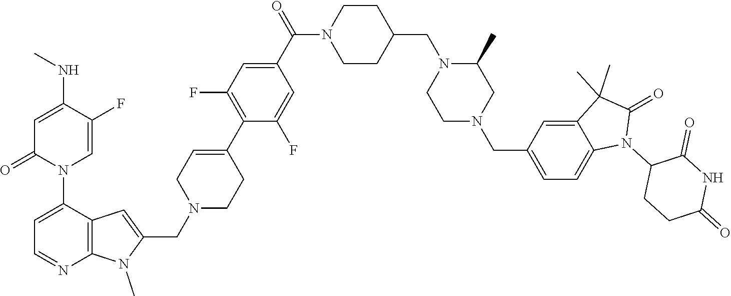 [SMILES: CNc1cc(=O)n(-c2ccnc3c2cc(CN2CC=C(c4c(F)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)cc1F]

### Example 3u77. 3-(5-(((S)-4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)-1,2,3,6-tetrahydropyridin-4-yl)benzoyl)piperidin-4-yl)methyl)-3-methylpiperazin-1-yl)methyl)-3,3-dimethyl-2-oxoindolin-1-yl)piperidine-2,6-dione

Example 3u77 (20 mg) was prepared similarly to Example 3u163 from Ints. 2G48/3E291 (0.10 g/0.13 g).

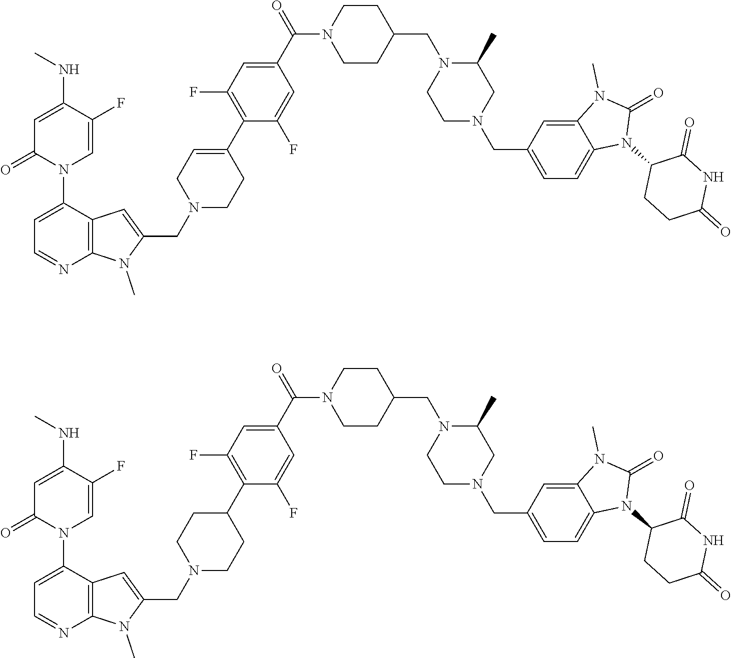 [SMILES: CNc1cc(=O)n(-c2ccnc3c2cc(CN2CC=C(c4c(F)cc(C(=O)N5CCC(CN6CCN(Cc7ccc8c(c7)n(C)c(=O)n8[C@H]7CCC(=O)NC7=O)C[C@@H]6C)CC5)cc4F)CC2)n3C)cc1F ; CNc1cc(=O)n(-c2ccnc3c2cc(CN2CCC(c4c(F)cc(C(=O)N5CCC(CN6CCN(Cc7ccc8c(c7)n(C)c(=O)n8[C@@H]7CCC(=O)NC7=O)C[C@@H]6C)CC5)cc4F)CC2)n3C)cc1F]

### Example 3u78 and 3u79. (S)-3-(5-(((S)-4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)-1,2,3,6-tetrahydropyridin-4-yl)benzoyl)piperidin-4-yl)methyl)-3-methylpiperazin-1-yl)methyl)-3,3-dimethyl-2-oxoindolin-1-yl)piperidine-2,6-dione and (R)-3-(5-(((S)-4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)-1,2,3,6-tetrahydropyridin-4-yl)benzoyl)piperidin-4-yl)methyl)-3-methylpiperazin-1-yl)methyl)-3,3-dimethyl-2-oxoindolin-1-yl)piperidine-2,6-dione

Example 3u77 (70 mg) was resolved by HPLC using a Chiralpak IK 250×30 mm 5 μm column with an eluent of 35% ACN, 35% IPA and 30% DCM (25 mL/min) to give Example 3u78 (15 mg, first peak) and Example 3u79 (13 mg, second peak). The absolute configurations for these compounds at the glutarimide chiral centers were not determined.

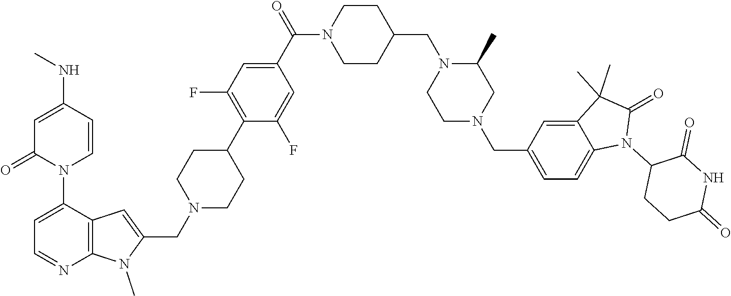 [SMILES: CNc1ccn(-c2ccnc3c2cc(CN2CCC(c4c(F)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)c(=O)c1]

### Example 3u80. 3-(5-(((S)-4-((1-(3,5-difluoro-4-(1-((S)-1-(1-methyl-4-(4-(methylamino)-2-oxopyridin-1 (2H)-yl)-1H-pyrrolo[2,3-b]pyridin-2-yl)ethyl)piperidin-4-yl)benzoyl)piperidin-4-yl)methyl)-3-methyl-piperazin-1-yl)methyl)-3,3-dimethyl-2-oxoindolin-1-yl)piperidine-2,6-dione

Example 3u80 (0.11 g) was prepared similarly to Example 3u163 from Ints. 3U72/3E291 (0.25 g/0.32 g).

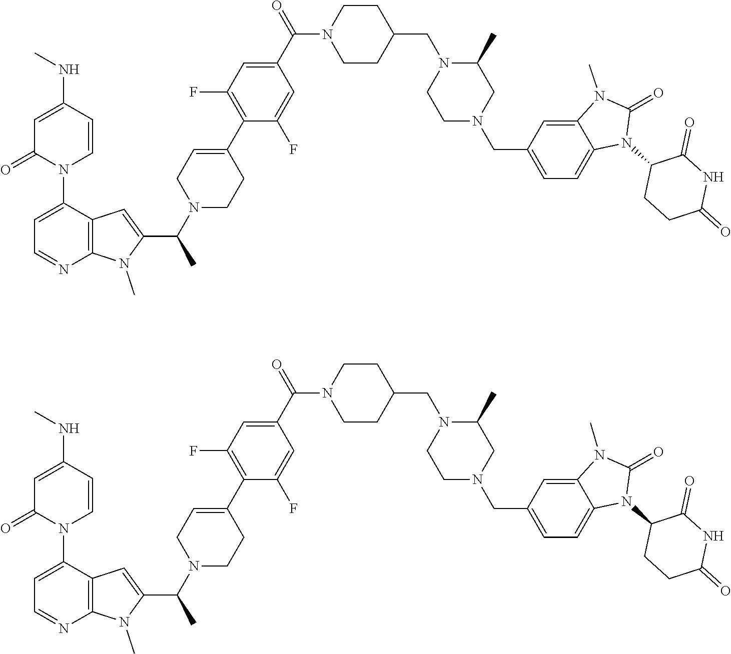 [SMILES: CNc1ccn(-c2ccnc3c2cc([C@H](C)N2CC=C(c4c(F)cc(C(=O)N5CCC(CN6CCN(Cc7ccc8c(c7)n(C)c(=O)n8[C@H]7CCC(=O)NC7=O)C[C@@H]6C)CC5)cc4F)CC2)n3C)c(=O)c1 ; CNc1ccn(-c2ccnc3c2cc([C@H](C)N2CC=C(c4c(F)cc(C(=O)N5CCC(CN6CCN(Cc7ccc8c(c7)n(C)c(=O)n8[C@@H]7CCC(=O)NC7=O)C[C@@H]6C)CC5)cc4F)CC2)n3C)c(=O)c1]

### Example 3u81 and 3u82. (S)-3-(5-(((S)-4-((1-(3,5-difluoro-4-(1-((S)-1-(1-methyl-4-(4-(methylamino)-2-oxopyridin-1 (2H)-yl)-1H-pyrrolo[2,3-b]pyridin-2-yl)ethyl)piperidin-4-yl)benzoyl)piperidin-4-yl)methyl)-3-methylpiperazin-1-yl)methyl)-3,3-dimethyl-2-oxoindolin-1-yl)piperidine-2,6-dione and (R)-3-(5-(((S)-4-((1-(3,5-difluoro-4-(1-((S)-1-(1-methyl-4-(4-(methylamino)-2-oxopyridin-1 (2H)-yl)-1H-pyrrolo[2,3-b]pyridin-2-yl)ethyl)piperidin-4-yl)benzoyl)piperidin-4-yl)methyl)-3-methylpiperazin-1-yl)methyl)-3,3-dimethyl-2-oxoindolin-1-yl)piperidine-2,6-dione

Example 3u80 (90 mg) was resolved by HPLC using a CELLULOSE-SC 250×30 mm 5 μm column with an eluent of 35% ACN, 35% IPA and 30% DCM (40 mL/min) to give Example 3u81 (10 mg, first peak) and Example 3u82 (12 mg, second peak). The absolute configurations for these compounds at the glutarimide chiral centers were not determined.

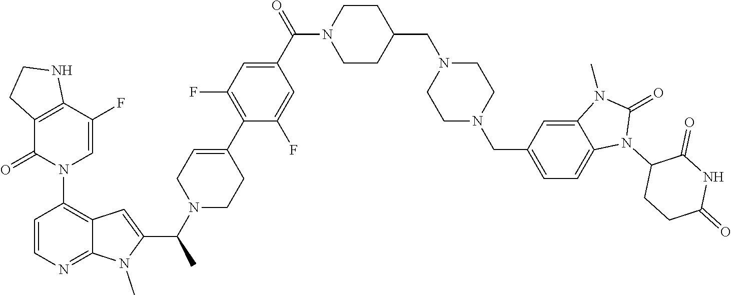 [SMILES: C[C@@H](c1cc2c(-n3cc(F)c4c(c3=O)CCN4)ccnc2n1C)N1CC=C(c2c(F)cc(C(=O)N3CCC(CN4CCN(Cc5ccc6c(c5)n(C)c(=O)n6C5CCC(=O)NC5=O)CC4)CC3)cc2F)CC1]

### Example 3u83. 3-(5-((4-((1-(3,5-difluoro-4-(1-((S)-1-(4-(7-fluoro-4-oxo-1,2,3,4-tetrahydro-5H-pyrrolo[3,2-c]pyridin-5-yl)-1-methyl-1H-pyrrolo[2,3-b]pyridin-2-yl)ethyl)-1,2,3,6-tetrahydropyridin-4-yl)benzoyl)piperidin-4-yl)methyl)piperazin-1-yl)methyl)-3-methyl-2-oxo-2,3-dihydro-1H-benzo[d]-imidazol-1-yl)piperidine-2,6-dione

Example 3u82 (28 mg) was prepared similarly to Example 3u163 from Ints. 2G11/3E1 (0.10 g/0.13 g).

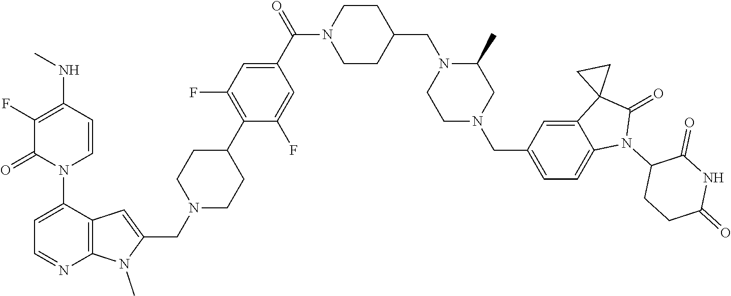 [SMILES: CNc1ccn(-c2ccnc3c2cc(CN2CCC(c4c(F)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)c(=O)c1F]

### Example 3u84. 3-(5′-(((S)-4-((1-(3,5-difluoro-4-(1-((4-(3-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)methyl)-3-methylpiperazin-1-yl)methyl)-2′-oxospiro[cyclopropane-1,3′-indolin]-1′-yl)piperidine-2,6-dione

Example 3u84 (32 mg) was prepared similarly to Example 3u163 from Ints. 3U78/3C42 (0.10 g/0.16 g).

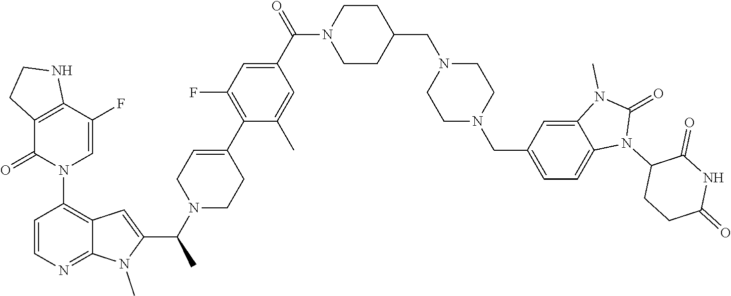 [SMILES: Cc1cc(C(=O)N2CCC(CN3CCN(Cc4ccc5c(c4)n(C)c(=O)n5C4CCC(=O)NC4=O)CC3)CC2)cc(F)c1C1=CCN([C@@H](C)c2cc3c(-n4cc(F)c5c(c4=O)CCN5)ccnc3n2C)CC1]

### Example 3u85. 3-(5-((4-((1-(3-fluoro-4-(1-((S)-1-(4-(7-fluoro-4-oxo-1,2,3,4-tetrahydro-5H-pyrrolo[3,2-c]pyridin-5-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)methyl)-3-methyl-2-oxo-2,3-dihydro-1H-benzo[d]imidazol-1-yl)piperidine-2,6-dione

Example 3u85 (16 mg) was prepared similarly to Example 3u163 from Ints. 2G27/3E1 (0.10 g/0.11 g).

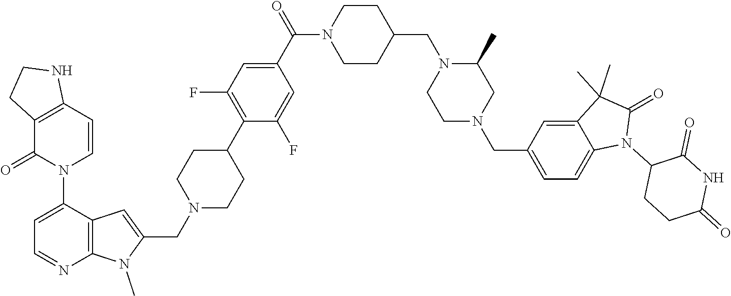 [SMILES: C[C@H]1CN(Cc2ccc3c(c2)C(C)(C)C(=O)N3C2CCC(=O)NC2=O)CCN1CC1CCN(C(=O)c2cc(F)c(C3CCN(Cc4cc5c(-n6ccc7c(c6=O)CCN7)ccnc5n4C)CC3)c(F)c2)CC1]

### Example 3u86. 3-(5-(((S)-4-((1-(3,5-difluoro-4-(1-((1-methyl-4-(4-oxo-1,2,3,4-tetrahydro-5H-pyrrolo[3,2-c]pyridin-5-yl)-1H-pyrrolo[2,3-b]pyridin-2-yl)methyl)piperidin-4-yl)benzoyl)piperidin-4-yl)methyl)-3-methylpiperazin-1-yl)methyl)-3,3-dimethyl-2-oxoindolin-1-yl)piperidine-2,6-dione

Example 3u86 (50 mg) was prepared similarly to Example 3u163 from Ints. 2G57/3E291 (0.30 g/0.32 g).

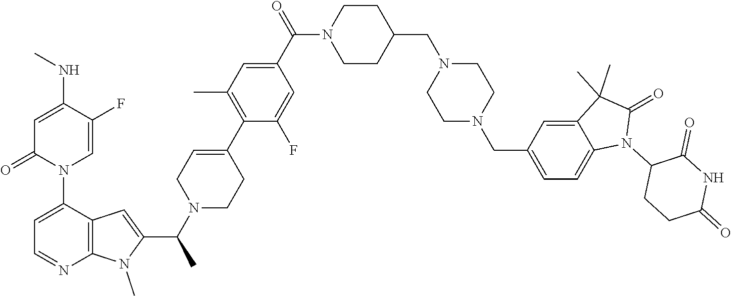 [SMILES: CNc1cc(=O)n(-c2ccnc3c2cc([C@H](C)N2CC=C(c4c(C)cc(C(=O)N5CCC(CN6CCN(Cc7ccc8c(c7)C(C)(C)C(=O)N8C7CCC(=O)NC7=O)CC6)CC5)cc4F)CC2)n3C)cc1F]

### Example 3u87. 3-(5-((4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)methyl)-3,3-dimethyl-2-oxoindolin-1-yl)piperidine-2,6-dione

Example 3u87 (0.14 g) was prepared similarly to Example 3u163 from Ints. 2G45/3U7 (0.30 g/0.42 g).

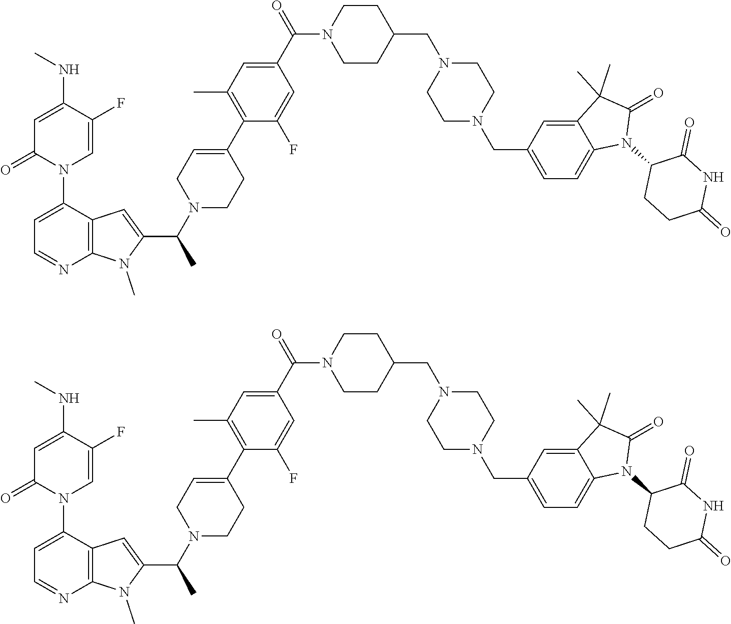 [SMILES: CNc1cc(=O)n(-c2ccnc3c2cc([C@H](C)N2CC=C(c4c(C)cc(C(=O)N5CCC(CN6CCN(Cc7ccc8c(c7)C(C)(C)C(=O)N8[C@H]7CCC(=O)NC7=O)CC6)CC5)cc4F)CC2)n3C)cc1F ; CNc1cc(=O)n(-c2ccnc3c2cc([C@H](C)N2CC=C(c4c(C)cc(C(=O)N5CCC(CN6CCN(Cc7ccc8c(c7)C(C)(C)C(=O)N8[C@@H]7CCC(=O)NC7=O)CC6)CC5)cc4F)CC2)n3C)cc1F]

### Example 3u88 and 3u89. (S)-3-(5-((4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)methyl)-3,3-dimethyl-2-oxoindolin-1-yl)piperidine-2,6-dione and (R)-3-(5-((4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)methyl)-3,3-dimethyl-2-oxoindolin-1-yl)piperidine-2,6-dione

Example 3u87 (0.10 g) was resolved by HPLC using a Chiralpak IC 250×10 mm 5 μm column with an eluent of 35% ACN, 35% IPA and 30% DCM (6 mL/min) to give Example 3u88 (15 mg, first peak) and Example 3u89 (31 mg, second peak). The absolute configurations for these compounds at the glutarimide chiral centers were not determined.

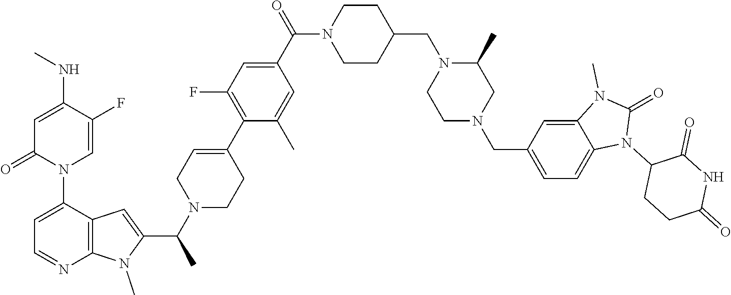 [SMILES: CNc1cc(=O)n(-c2ccnc3c2cc([C@H](C)N2CC=C(c4c(C)cc(C(=O)N5CCC(CN6CCN(Cc7ccc8c(c7)n(C)c(=O)n8C7CCC(=O)NC7=O)C[C@@H]6C)CC5)cc4F)CC2)n3C)cc1F]

### Example 3u90. 3-(5-(((S)-4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione

Example 3u90 (20 mg) was prepared similarly to Example 3u163 from Ints. 2G45/3E68 (0.20 g/0.26 g).

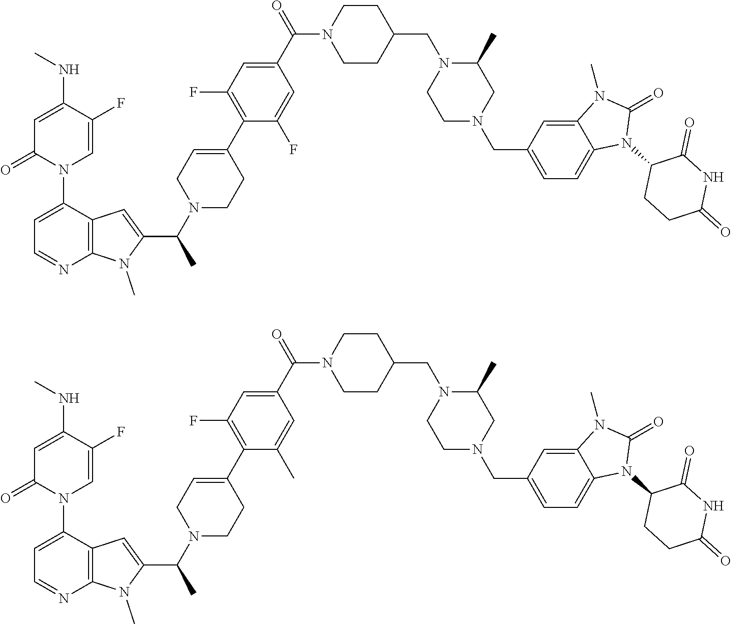 [SMILES: CNc1cc(=O)n(-c2ccnc3c2cc([C@H](C)N2CC=C(c4c(F)cc(C(=O)N5CCC(CN6CCN(Cc7ccc8c(c7)n(C)c(=O)n8[C@H]7CCC(=O)NC7=O)C[C@@H]6C)CC5)cc4F)CC2)n3C)cc1F ; CNc1cc(=O)n(-c2ccnc3c2cc([C@H](C)N2CC=C(c4c(C)cc(C(=O)N5CCC(CN6CCN(Cc7ccc8c(c7)n(C)c(=O)n8[C@@H]7CCC(=O)NC7=O)C[C@@H]6C)CC5)cc4F)CC2)n3C)cc1F]

### Example 3u91 and 3u92. (S)-3-(5-(((S)-4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione and (R)-3-(5-(((S)-4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione

Example 3u90 (60 mg) was resolved by HPLC using a Chiralpak IK 250×10 mm 5 μm column with an eluent of 40% ACN, 40% IPA and 20% DCM (3 mL/min) to give Example 3u91 (19 mg, first peak) and Example 3u92 (20 mg, second peak). The absolute configurations for these compounds at the glutarimide chiral centers were not determined.

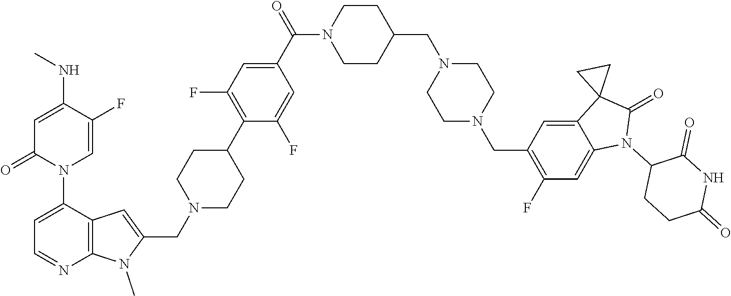 [SMILES: CNc1cc(=O)n(-c2ccnc3c2cc(CN2CCC(c4c(F)cc(C(=O)N5CCC(CN6CCN(Cc7cc8c(cc7F)N(C7CCC(=O)NC7=O)C(=O)C87CC7)CC6)CC5)cc4F)CC2)n3C)cc1F]

### Example 3u93. 3-(5′-((4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)methyl)piperazin-1-yl)methyl)-6′-fluoro-2′-oxospiro[cyclopropane-1,3′-indolin]-1′-yl)piperidine-2,6-dione

Example 3u93 (0.14 g) was prepared similarly to Example 3u163 from Ints. 2G58/3C4 (0.30 g/0.36 g).

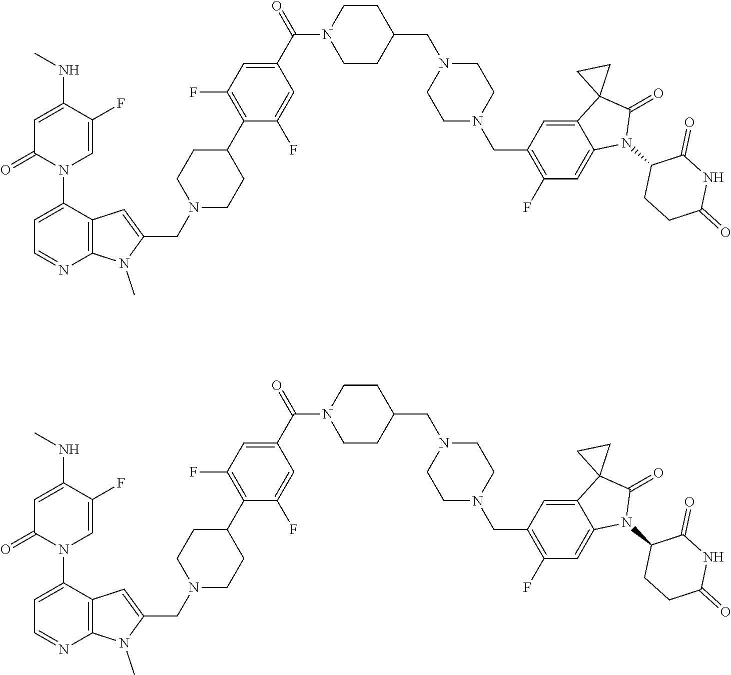 [SMILES: CNc1cc(=O)n(-c2ccnc3c2cc(CN2CCC(c4c(F)cc(C(=O)N5CCC(CN6CCN(Cc7cc8c(cc7F)N([C@H]7CCC(=O)NC7=O)C(=O)C87CC7)CC6)CC5)cc4F)CC2)n3C)cc1F ; CNc1cc(=O)n(-c2ccnc3c2cc(CN2CCC(c4c(F)cc(C(=O)N5CCC(CN6CCN(Cc7cc8c(cc7F)N([C@@H]7CCC(=O)NC7=O)C(=O)C87CC7)CC6)CC5)cc4F)CC2)n3C)cc1F]

### Example 3u94 and 3u95. (S)-3-(5′-((4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)methyl)piperazin-1-yl)methyl)-6′-fluoro-2′-oxospiro[cyclopropane-1,3′-indolin]-1′-yl)piperidine-2,6-dione and (R)-3-(5′-((4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)methyl)piperazin-1-yl)methyl)-6′-fluoro-2′-oxospiro[cyclopropane-1,3′-indolin]-1′-yl)piperidine-2,6-dione

Example 3u93 (84 mg) was resolved by HPLC using a Chiralpak IK 250×10 mm 5 μm column with an eluent of 50% IPA and 50% DCM (6 mL/min) to give Example 3u94 (23 mg, first peak) and Example 3u95 (25 mg, second peak). The absolute configurations for these compounds at the glutarimide chiral centers were not determined.

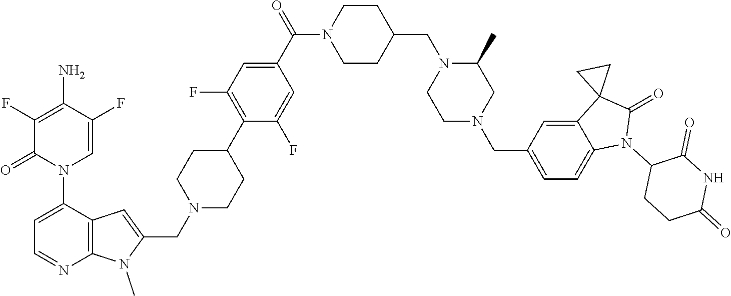 [SMILES: C[C@H]1CN(Cc2ccc3c(c2)C2(CC2)C(=O)N3C2CCC(=O)NC2=O)CCN1CC1CCN(C(=O)c2cc(F)c(C3CCN(Cc4cc5c(-n6cc(F)c(N)c(F)c6=O)ccnc5n4C)CC3)c(F)c2)CC1]

### Example 3u96. 3-(5′-(((S)-4-((1-(4-(1-((4-(4-amino-3,5-difluoro-2-oxopyridin-1 (2H)-yl)-1-methyl-1H-pyrrolo[2,3-b]pyridin-2-yl)methyl)piperidin-4-yl)-3,5-difluorobenzoyl)piperidin-4-yl)methyl)-3-methylpiperazin-1-yl)methyl)-2′-oxospiro[cyclopropane-1,3′-indolin]-1′-yl)piperidine-2,6-dione

Example 3u96 (10 mg) was prepared similarly to Example 3u163 from Ints. 3U28/3C42 (0.10 g/0.15 g).

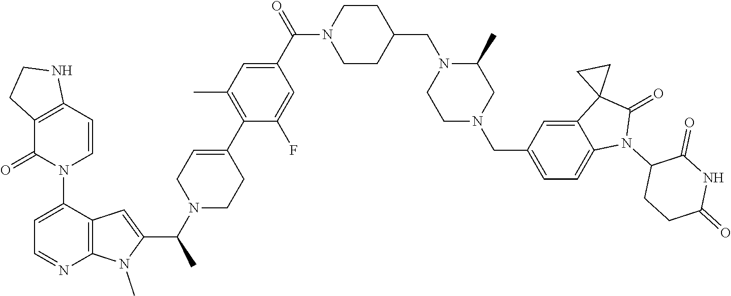 [SMILES: Cc1cc(C(=O)N2CCC(CN3CCN(Cc4ccc5c(c4)C4(CC4)C(=O)N5C4CCC(=O)NC4=O)C[C@@H]3C)CC2)cc(F)c1C1=CCN([C@@H](C)c2cc3c(-n4ccc5c(c4=O)CCN5)ccnc3n2C)CC1]

### Example 3u165. 3-(5-(((S)-4-((1-(3-fluoro-5-methyl-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-3,3-dimethyl-2-oxoindolin-1-yl)piperidine-2,6-dione

Example 3u165 (45 mg) was prepared similarly to Example 3u163 from Ints. 2G53/3E291 (0.15 g/0.18 g).

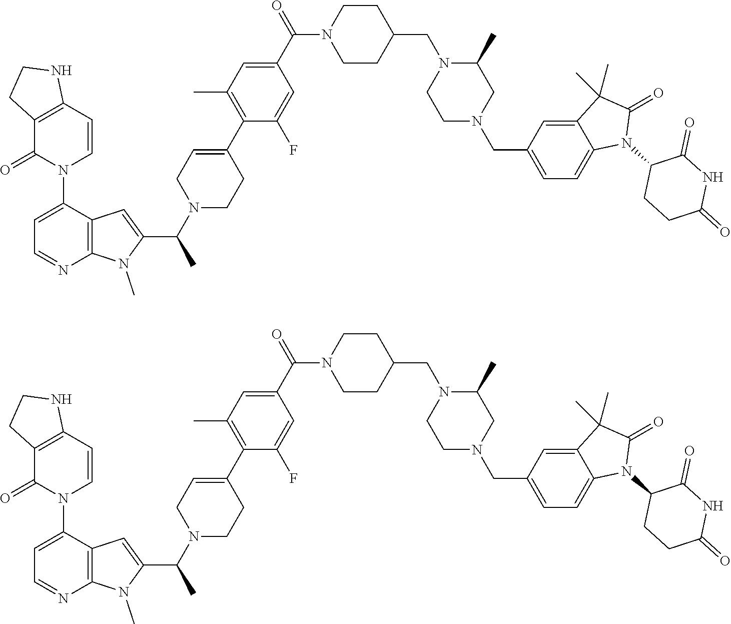 [SMILES: Cc1cc(C(=O)N2CCC(CN3CCN(Cc4ccc5c(c4)C(C)(C)C(=O)N5[C@H]4CCC(=O)NC4=O)C[C@@H]3C)CC2)cc(F)c1C1=CCN([C@@H](C)c2cc3c(-n4ccc5c(c4=O)CCN5)ccnc3n2C)CC1 ; Cc1cc(C(=O)N2CCC(CN3CCN(Cc4ccc5c(c4)C(C)(C)C(=O)N5[C@@H]4CCC(=O)NC4=O)C[C@@H]3C)CC2)cc(F)c1C1=CCN([C@@H](C)c2cc3c(-n4ccc5c(c4=O)CCN5)ccnc3n2C)CC1]

### Example 3u97 and 3u98. (S)-3-(5-(((S)-4-((1-(3-fluoro-5-methyl-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-3,3-dimethyl-2-oxoindolin-1-yl)piperidine-2,6-dione and (R)-3-(5-(((S)-4-((1-(3-fluoro-5-methyl-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-3,3-dimethyl-2-oxoindolin-1-yl)piperidine-2,6-dione

Example 3u165 (45 mg) was resolved by HPLC using a Chiralpak IK 250×10 mm 5 μm column with an eluent of 35% ACN, 35% IPA and 30% DCM (4 mL/min) to give Example 3u98 (4 mg, first peak) and Example 3u97 (8 mg, second peak). The absolute configurations for these compounds at the glutarimide chiral centers were not determined.

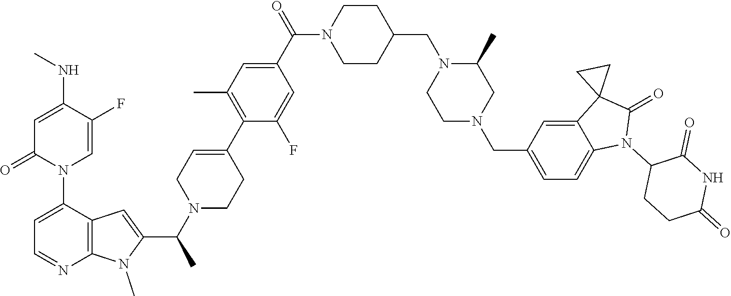 [SMILES: CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)cc1F]

### Example 3u164. 3-(5′-(((S)-4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methyl-benzoyl)piperidin-4-yl)methyl)-3-methylpiperazin-1-yl)methyl)-2′-oxospiro[cyclopropane-1,3′-indolin]-1′-yl)piperidine-2,6-dione

Example 3u164 (80 mg) was prepared similarly to Example 3u163 from Ints. 2G45/3C42 (0.25 g/0.36 g).

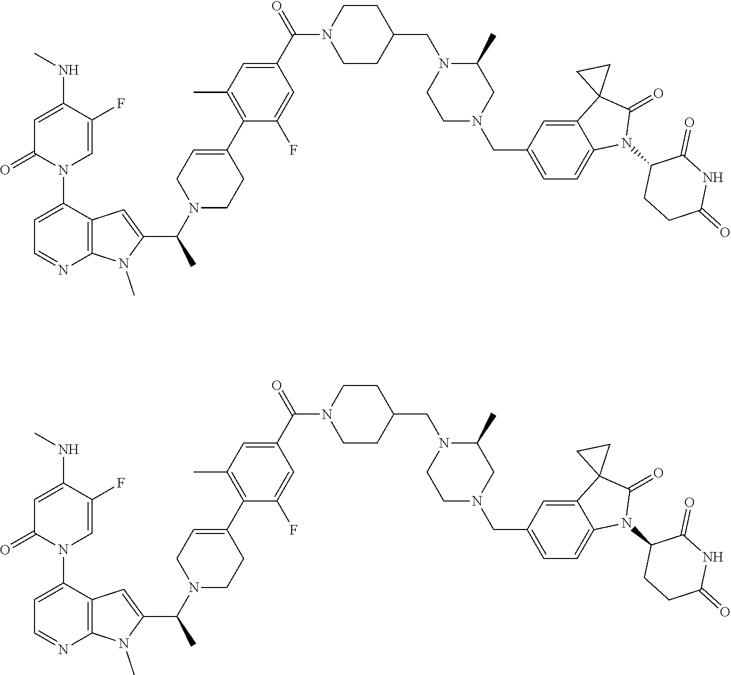 [SMILES: CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8[C@H]7CCC(=O)NC7=O)C[C@@H]6C)CC5)cc4F)CC2)n3C)cc1F ; CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8[C@@H]7CCC(=O)NC7=O)C[C@@H]6C)CC5)cc4F)CC2)n3C)cc1F]

### Example 3u99 and 3u100. (S)-3-(5′-(((S)-4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)-3-methylpiperazin-1-yl)methyl)-2′-oxospiro[cyclopropane-1,3′-indolin]-1′-yl)piperidine-2,6-dione and (R)-3-(5′-(((S)-4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)-3-methylpiperazin-1-yl)methyl)-2′-oxospiro[cyclopropane-1,3′-indolin]-1′-yl)piperidine-2,6-dione

Example 3u164 (0.10 g) was resolved by HPLC using a Chiralpak IK 250×30 mm 5 μm column with an eluent of 35% ACN, 35% IPA and 30% DCM (22 mL/min) to give Example 3u99 (27 mg, first peak) and Example 3u100 (22 mg, second peak). The absolute configurations for these compounds at the glutarimide chiral centers were not determined.

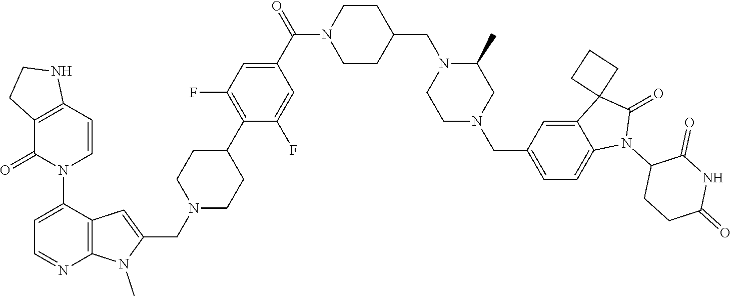 [SMILES: C[C@H]1CN(Cc2ccc3c(c2)C2(CCC2)C(=O)N3C2CCC(=O)NC2=O)CCN1CC1CCN(C(=O)c2cc(F)c(C3CCN(Cc4cc5c(-n6ccc7c(c6=O)CCN7)ccnc5n4C)CC3)c(F)c2)CC1]

### Example 3u101. 3-(5′-(((S)-4-((1-(3,5-difluoro-4-(1-((1-methyl-4-(4-oxo-1,2,3,4-tetrahydro-5H-pyrrolo[3,2-c]pyridin-5-yl)-1H-pyrrolo[2,3-b]pyridin-2-yl)methyl)piperidin-4-yl)benzoyl)piperidin-4-yl)methyl)-3-methylpiperazin-1-yl)methyl)-2′-oxospiro[cyclobutane-1,3′-indolin]-1′-yl)piperidine-2,6-dione

Example 3u101 (90 mg) was prepared similarly to Example 3u163 from Ints. 2G57/3U30 (0.30 g/0.33 g).

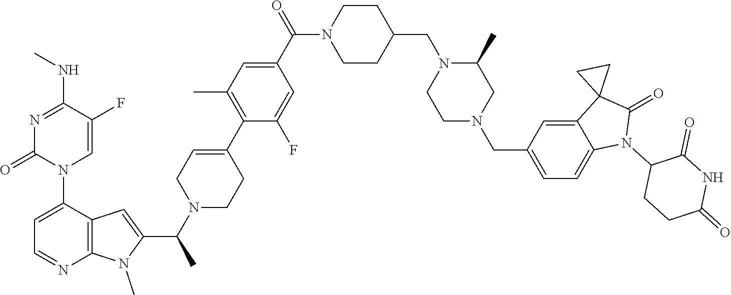 [SMILES: CNc1nc(=O)n(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)cc1F]

### Example 3u102. 3-(5′-(((S)-4-((1-(3-fluoro-4-(1-((S)-1-(4-(5-fluoro-4-(methylamino)-2-oxopyrimidin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)-3-methylpiperazin-1-yl)methyl)-2′-oxospiro[cyclopropane-1,3′-indolin]-1′-yl)piperidine-2,6-dione

Example 3u102 (21 mg) was prepared similarly to Example 3u163 from Ints. 2G20/3C42 (0.10 g/0.13 g).

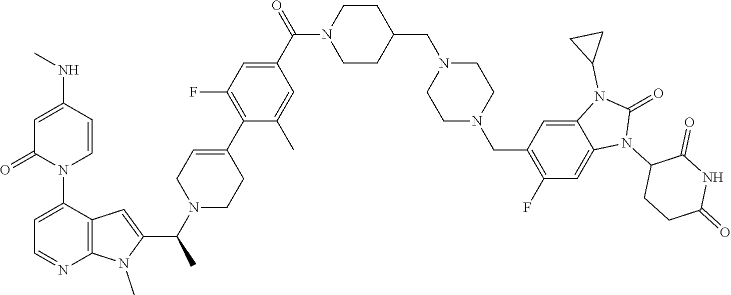 [SMILES: CNc1ccn(-c2ccnc3c2cc([C@H](C)N2CC=C(c4c(C)cc(C(=O)N5CCC(CN6CCN(Cc7cc8c(cc7F)n(C7CCC(=O)NC7=O)c(=O)n8C7CC7)CC6)CC5)cc4F)CC2)n3C)c(=O)c1]

### Example 3u103. 3-(3-cyclopropyl-6-fluoro-5-((4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)methyl)-2-oxo-2,3-dihydro-1H-benzo[d]imidazol-1-yl)piperidine-2,6-dione

Example 3u103 (60 mg) was prepared similarly to Example 3u163 from Ints. 2G46/3E148 (0.30 g/0.40 g).

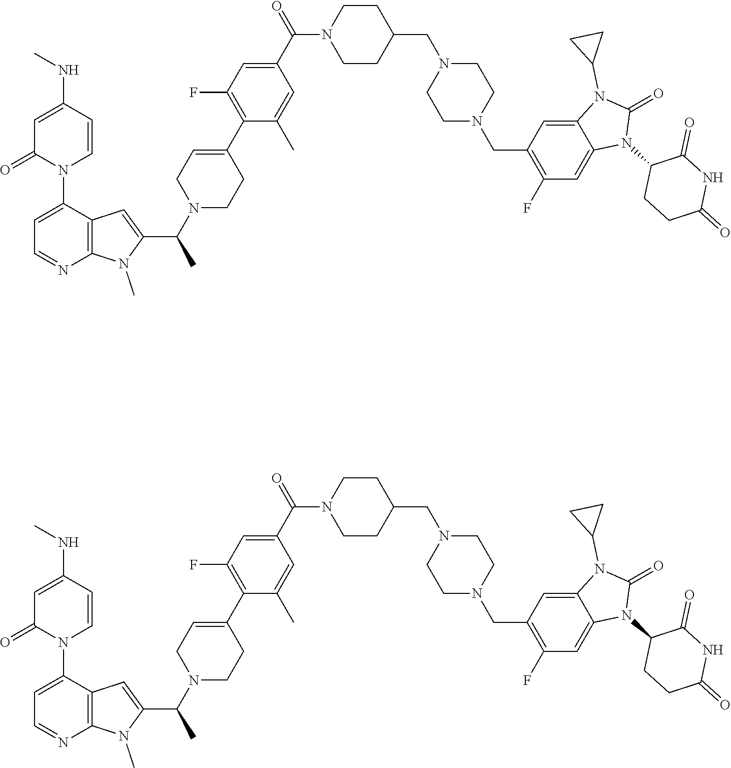 [SMILES: CNc1ccn(-c2ccnc3c2cc([C@H](C)N2CC=C(c4c(C)cc(C(=O)N5CCC(CN6CCN(Cc7cc8c(cc7F)n([C@H]7CCC(=O)NC7=O)c(=O)n8C7CC7)CC6)CC5)cc4F)CC2)n3C)c(=O)c1 ; CNc1ccn(-c2ccnc3c2cc([C@H](C)N2CC=C(c4c(C)cc(C(=O)N5CCC(CN6CCN(Cc7cc8c(cc7F)n([C@@H]7CCC(=O)NC7=O)c(=O)n8C7CC7)CC6)CC5)cc4F)CC2)n3C)c(=O)c1]

### Example 3u104 and 3u105. (S)-3-(3-cyclopropyl-6-fluoro-5-((4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)methyl)-2-oxo-2,3-dihydro-1H-benzo[d]imidazol-1-yl)piperidine-2,6-dione and (R)-3-(3-cyclopropyl-6-fluoro-5-((4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)methyl)-2-oxo-2,3-dihydro-1H-benzo[d]imidazol-1-yl)piperidine-2,6-dione

Example 3u103 (45 mg) was resolved by HPLC using a Chiralpak IK 250×10 mm 5 μm column with an eluent of 35% ACN, 35% IPA and 30% DCM (4 mL/min) to give Example 3u104 (15 mg, first peak) and Example 3u105 (14 mg, second peak). The absolute configurations for these compounds at the glutarimide chiral centers were not determined.

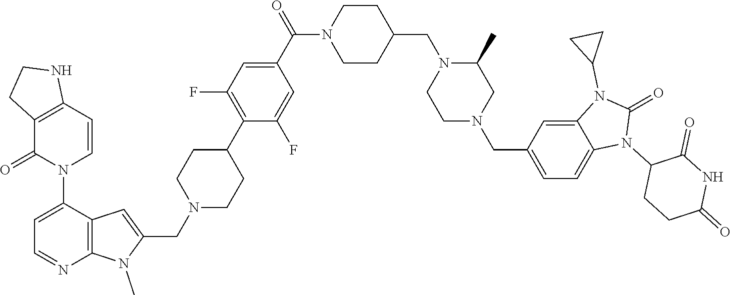 [SMILES: C[C@H]1CN(Cc2ccc3c(c2)n(C2CC2)c(=O)n3C2CCC(=O)NC2=O)CCN1CC1CCN(C(=O)c2cc(F)c(C3CCN(Cc4cc5c(-n6ccc7c(c6=O)CCN7)ccnc5n4C)CC3)c(F)c2)CC1]

### Example 3u106. 3-(3-cyclopropyl-5-(((S)-4-((1-(3,5-difluoro-4-(1-((1-methyl-4-(4-oxo-1,2,3,4-tetrahydro-5H-pyrrolo[3,2-c]pyridin-5-yl)-1H-pyrrolo[2,3-b]pyridin-2-yl)methyl)piperidin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione

Example 3u106 (85 mg) was prepared similarly to Example 3u163 from Ints. 2G57/3E78 (0.35 g/0.35 g).

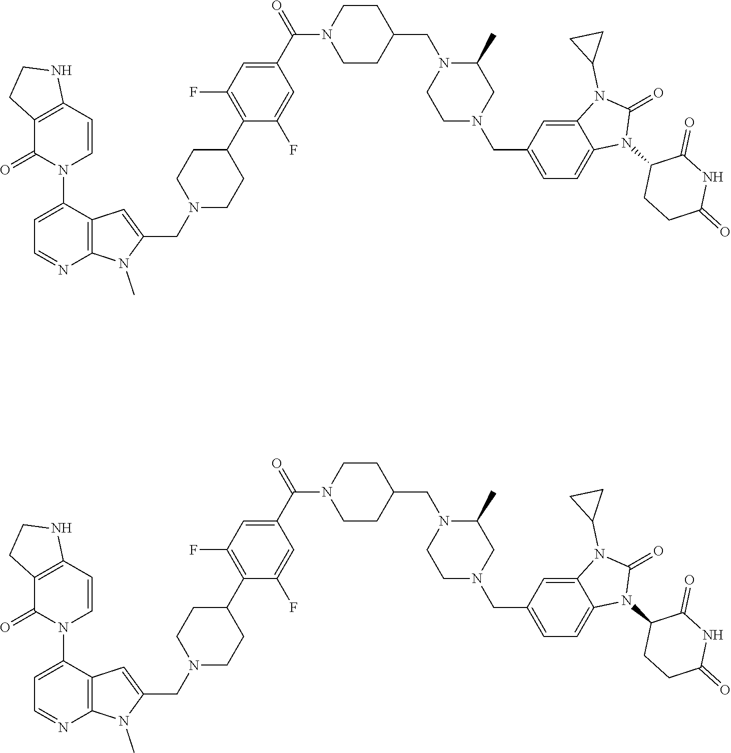 [SMILES: C[C@H]1CN(Cc2ccc3c(c2)n(C2CC2)c(=O)n3[C@H]2CCC(=O)NC2=O)CCN1CC1CCN(C(=O)c2cc(F)c(C3CCN(Cc4cc5c(-n6ccc7c(c6=O)CCN7)ccnc5n4C)CC3)c(F)c2)CC1 ; C[C@H]1CN(Cc2ccc3c(c2)n(C2CC2)c(=O)n3[C@@H]2CCC(=O)NC2=O)CCN1CC1CCN(C(=O)c2cc(F)c(C3CCN(Cc4cc5c(-n6ccc7c(c6=O)CCN7)ccnc5n4C)CC3)c(F)c2)CC1]

### Example 3u107 and 3u108. (S)-3-(3-cyclopropyl-5-(((S)-4-((1-(3,5-difluoro-4-(1-((1-methyl-4-(4-oxo-1,2,3,4-tetrahydro-5H-pyrrolo[3,2-c]pyridin-5-yl)-1H-pyrrolo[2,3-b]pyridin-2-yl)methyl)piperidin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione and (R)-3-(3-cyclopropyl-5-(((S)-4-((1-(3,5-difluoro-4-(1-((1-methyl-4-(4-oxo-1,2,3,4-tetrahydro-5H-pyrrolo[3,2-c]pyridin-5-yl)-1H-pyrrolo[2,3-b]pyridin-2-yl)methyl)piperidin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione

Example 3u106 (45 mg) was resolved by HPLC using a CELLULOSE SZ 250×10 mm 5 μm column with an eluent of 35% ACN, 35% IPA and 30% DCM (6 mL/min) to give Example 3u107 (21 mg, first peak) and Example 3u108 (21 mg, second peak). The absolute configurations for these compounds at the glutarimide chiral centers were not determined.

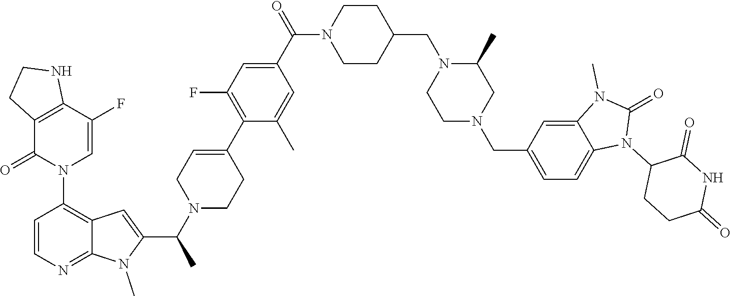 [SMILES: Cc1cc(C(=O)N2CCC(CN3CCN(Cc4ccc5c(c4)n(C)c(=O)n5C4CCC(=O)NC4=O)C[C@@H]3C)CC2)cc(F)c1C1=CCN([C@@H](C)c2cc3c(-n4cc(F)c5c(c4=O)CCN5)ccnc3n2C)CC1]

### Example 3u109. 3-(5-(((S)-4-((1-(3-fluoro-4-(1-((S)-1-(4-(7-fluoro-4-oxo-1,2,3,4-tetrahydro-5H-pyrrolo[3,2-c]pyridin-5-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione

Example 3u109 (20 mg) was prepared similarly to Example 3u163 from Ints. 2G27/3E78 (0.10 g/0.12 g).

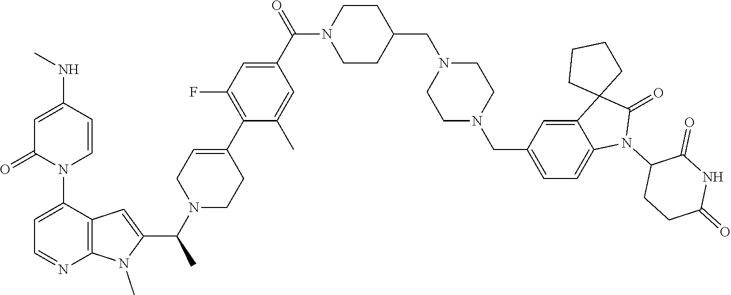 [SMILES: CNc1ccn(-c2ccnc3c2cc([C@H](C)N2CC=C(c4c(C)cc(C(=O)N5CCC(CN6CCN(Cc7ccc8c(c7)C7(CCCC7)C(=O)N8C7CCC(=O)NC7=O)CC6)CC5)cc4F)CC2)n3C)c(=O)c1]

### Example 3u110. 3-(5′-((4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)methyl)-2′-oxospiro[cyclopentane-1,3′-indolin]-1′-yl)piperidine-2,6-dione

Example 3u110 (4 mg) was prepared similarly to Example 3u163 from Ints. 3U35/2G46 (81 mg/62 mg) using HATU (48 mg) instead of T3P.

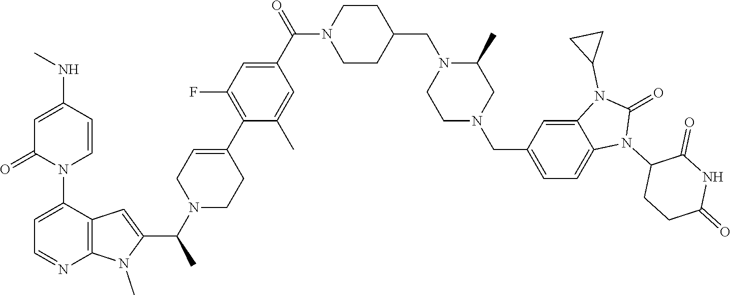 [SMILES: CNc1ccn(-c2ccnc3c2cc([C@H](C)N2CC=C(c4c(C)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)c(=O)c1]

### Example 3u111. 3-(3-cyclopropyl-5-(((S)-4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione

Example 3u111 (85 mg) was prepared similarly to Example 3u163 from Ints. 2G46/3E78 (0.30 g/0.45 g).

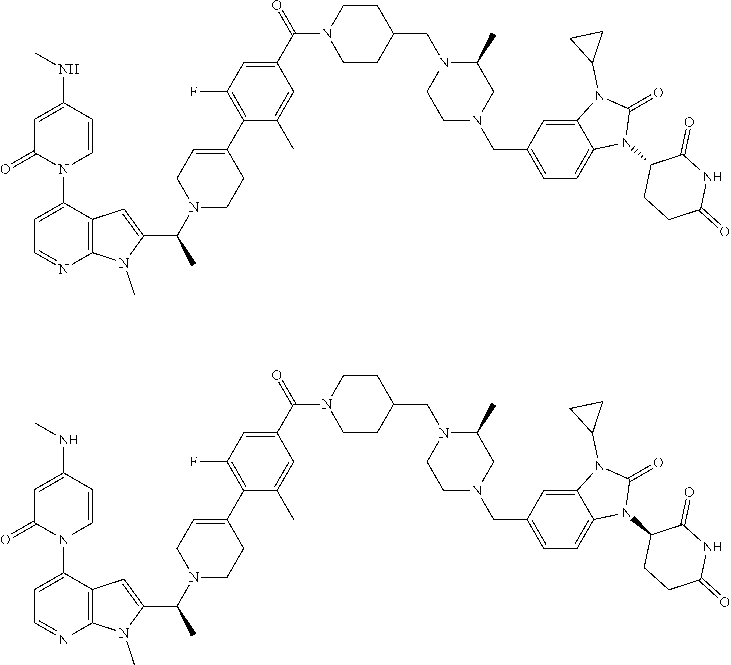 [SMILES: CNc1ccn(-c2ccnc3c2cc([C@H](C)N2CC=C(c4c(C)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)c(=O)c1 ; CNc1ccn(-c2ccnc3c2cc([C@H](C)N2CC=C(c4c(C)cc(C(=O)N5CCC(CN6CCN(Cc7ccc8c(c7)n(C7CC7)c(=O)n8[C@@H]7CCC(=O)NC7=O)C[C@@H]6C)CC5)cc4F)CC2)n3C)c(=O)c1]

### Example 3u112 and 3u113. (S)-3-(3-cyclopropyl-5-(((S)-4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione and (R)-3-(3-cyclopropyl-5-(((S)-4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione

Example 3u111 (85 mg) was resolved by HPLC using a Chiralpak IK 250×30 mm 5 μm column with an eluent of 35% ACN, 35% IPA and 30% DCM (48 mL/min) to give Example 3u112 (25 mg, first peak) and Example 3u113 (24 mg, second peak). The absolute configurations for these compounds at the glutarimide chiral centers were not determined.

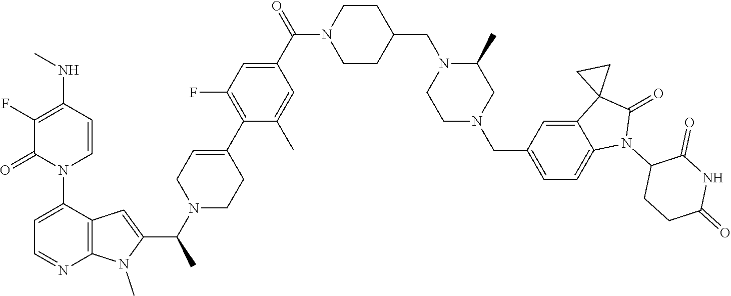 [SMILES: CNc1ccn(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)c(=O)c1F]

### Example 3u114. 3-(5′-(((S)-4-((1-(3-fluoro-4-(1-((S)-1-(4-(3-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methyl-benzoyl)piperidin-4-yl)methyl)-3-methylpiperazin-1-yl)methyl)-2′-oxospiro[cyclopropane-1,3′-indolin]-1′-yl)piperidine-2,6-dione

Example 3u114 (32 mg) was prepared similarly to Example 3u163 from Ints. 3U39/3C42 (0.11 g/0.16 g).

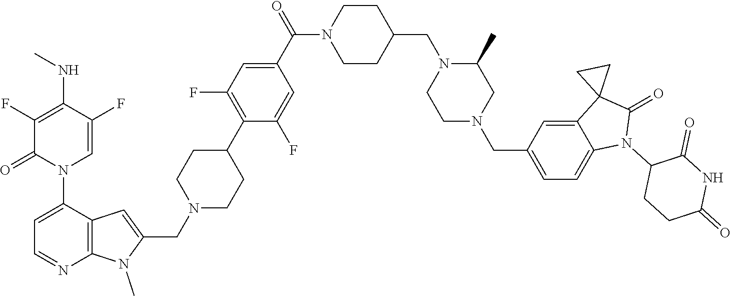 [SMILES: CNc1c(F)cn(-c2ccnc3c2cc(CN2CCC(c4c(F)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)c(=O)c1F]

### Example 3u115. 3-(5′-(((S)-4-((1-(4-(1-((4-(3,5-difluoro-4-(methylamino)-2-oxopyridin-1 (2H)-yl)-1-methyl-1H-pyrrolo[2,3-b]pyridin-2-yl)methyl)piperidin-4-yl)-3,5-difluorobenzoyl)piperidin-4-yl)-methyl)-3-methylpiperazin-1-yl)methyl)-2′-oxospiro[cyclopropane-1,3′-indolin]-1′-yl)piperidine-2,6-dione

Example 3u115 (46 mg) was prepared similarly to Example 3u163 from Ints. 3U41/3C42 (0.10 g/0.21 g).

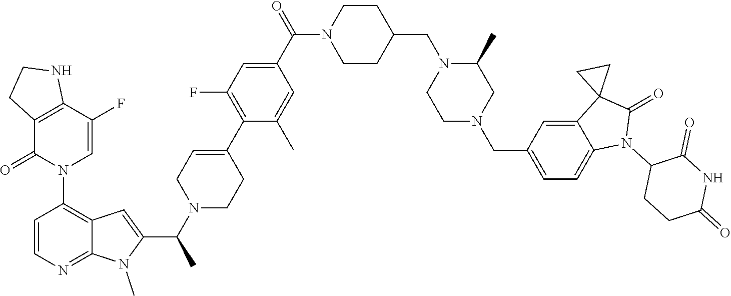 [SMILES: Cc1cc(C(=O)N2CCC(CN3CCN(Cc4ccc5c(c4)C4(CC4)C(=O)N5C4CCC(=O)NC4=O)C[C@@H]3C)CC2)cc(F)c1C1=CCN([C@@H](C)c2cc3c(-n4cc(F)c5c(c4=O)CCN5)ccnc3n2C)CC1]

### Example 3u116. 3-(5′-(((S)-4-((1-(3-fluoro-4-(1-((S)-1-(4-(7-fluoro-4-oxo-1,2,3,4-tetrahydro-5H-pyrrolo[3,2-c]pyridin-5-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)-3-methylpiperazin-1-yl)methyl)-2′-oxospiro[cyclopropane-1,3′-indolin]-1′-yl)piperidine-2,6-dione

Example 3u116 (20 mg) was prepared similarly to Example 3u163 from Ints. 2G27/3C42 (0.10 g/0.14 g).

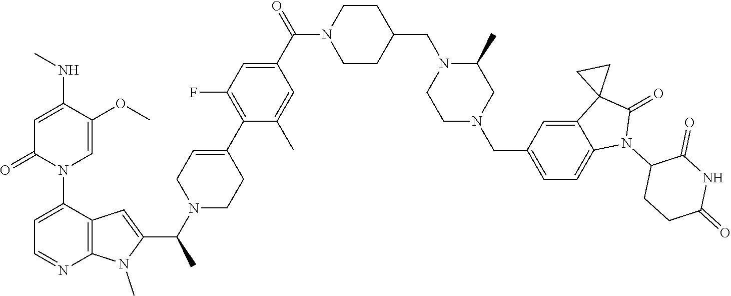 [SMILES: CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)cc1OC]

### Example 3u117. 3-(5′-(((S)-4-((1-(3-fluoro-4-(1-((S)-1-(4-(5-methoxy-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)-3-methylpiperazin-1-yl)methyl)-2′-oxospiro[cyclopropane-1,3′-indolin]-1′-yl)piperidine-2,6-dione

Example 3u117 (62 mg) was prepared similarly to Example 3u163 from Ints. 3U65/3C42 (0.12 g/0.17 g).

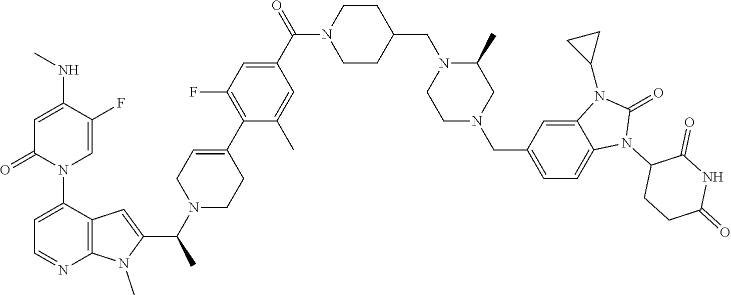 [SMILES: CNc1cc(=O)n(-c2ccnc3c2cc([C@H](C)N2CC=C(c4c(C)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)cc1F]

### Example 3u118. 3-(3-cyclopropyl-5-(((S)-4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)-3-methylpiperazin-1-yl)methyl)-2-oxo-2,3-dihydro-1H-benzo[d]imidazol-1-yl)piperidine-2,6-dione

Example 3u118 (0.14 g) was prepared similarly to Example 3u163 from Ints. 2G45/3E78 (0.30 g/0.48 g).

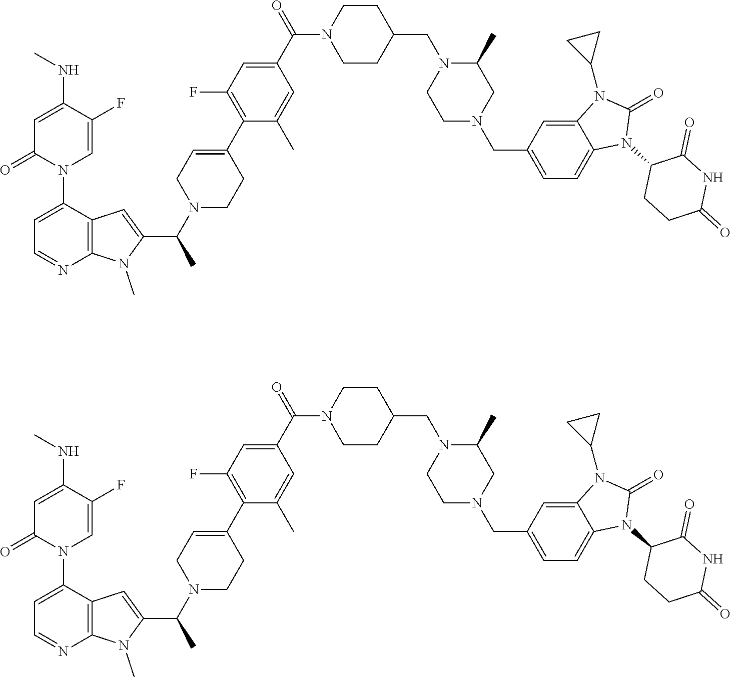 [SMILES: CNc1cc(=O)n(-c2ccnc3c2cc([C@H](C)N2CC=C(c4c(C)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)cc1F ; CNc1cc(=O)n(-c2ccnc3c2cc([C@H](C)N2CC=C(c4c(C)cc(C(=O)N5CCC(CN6CCN(Cc7ccc8c(c7)n(C7CC7)c(=O)n8[C@@H]7CCC(=O)NC7=O)C[C@@H]6C)CC5)cc4F)CC2)n3C)cc1F]

### Example 3u119 and 3u120. (S)-3-(3-cyclopropyl-5-(((S)-4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)-3-methylpiperazin-1-yl)methyl)-2-oxo-2,3-dihydro-1H-benzo[d]imidazol-1-yl)piperidine-2,6-dione and (R)-3-(3-cyclopropyl-5-(((S)-4-((1-(3-fluoro-4-(1-((S)-1-(4-(5-fluoro-4-(methylamino)-2-oxopyridin-1 (2H)-yl)-1-methyl-1H-pyrrolo-[2,3-b]pyridin-2-yl)ethyl)-1,2,3,6-tetrahydropyridin-4-yl)-5-methylbenzoyl)piperidin-4-yl)methyl)-3-methylpiperazin-1-yl)methyl)-2-oxo-2,3-dihydro-1H-benzo[d]imidazol-1-yl)piperidine-2,6-dione

Example 3u118 (0.12 g) was resolved by HPLC using a Chiralpak IK 250×10 mm 5 μm column with an eluent of 35% ACN, 35% IPA and 30% DCM (6 mL/min) to give Example 3u119 (42 mg, first peak) and Example 3u120 (35 mg, second peak). The absolute configurations for these compounds at the glutarimide chiral centers were not determined.

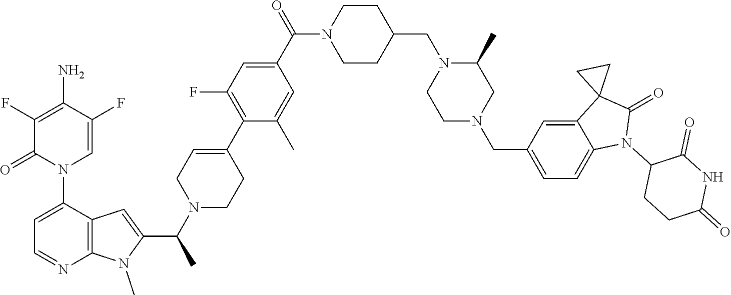 [SMILES: Cc1cc(C(=O)N2CCC(CN3CCN(Cc4ccc5c(c4)C4(CC4)C(=O)N5C4CCC(=O)NC4=O)C[C@@H]3C)CC2)cc(F)c1C1=CCN([C@@H](C)c2cc3c(-n4cc(F)c(N)c(F)c4=O)ccnc3n2C)CC1]

### Example 3u121. 3-(5′-(((S)-4-((1-(4-(1-((S)-1-(4-(4-amino-3,5-difluoro-2-oxopyridin-1 (2H)-yl)-1-methyl-1H-pyrrolo[2,3-b]pyridin-2-yl)ethyl)-1,2,3,6-tetrahydropyridin-4-yl)-3-fluoro-5-methyl-benzoyl)piperidin-4-yl)methyl)-3-methylpiperazin-1-yl)methyl)-2′-oxospiro[cyclopropane-1,3′-indolin]-1′-yl)piperidine-2,6-dione

Example 3u121 (20 mg) was prepared similarly to Example 3u163 from Ints. 3U43/3C42 (0.20 g/0.14 g).

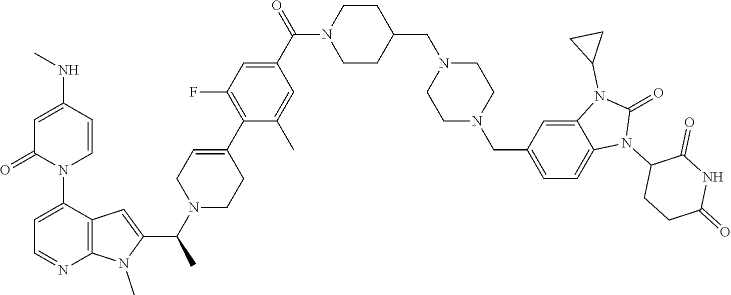 [SMILES: CNc1ccn(-c2ccnc3c2cc([C@H](C)N2CC=C(c4c(C)cc(C(=O)N5CCC(CN6CCN(Cc7ccc8c(c7)n(C7CC7)c(=O)n8C7CCC(=O)NC7=O)CC6)CC5)cc4F)CC2)n3C)c(=O)c1]

### Example 3u122. 3-(3-cyclopropyl-5-((4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)methyl)-2-oxo-2,3-dihydro-1H-benzo[d]imidazol-1-yl)piperidine-2,6-dione

Example 3u122 (0.15 g) was prepared similarly to Example 3u163 from Ints. 2G46/3E33 (0.30 g/0.36 g).

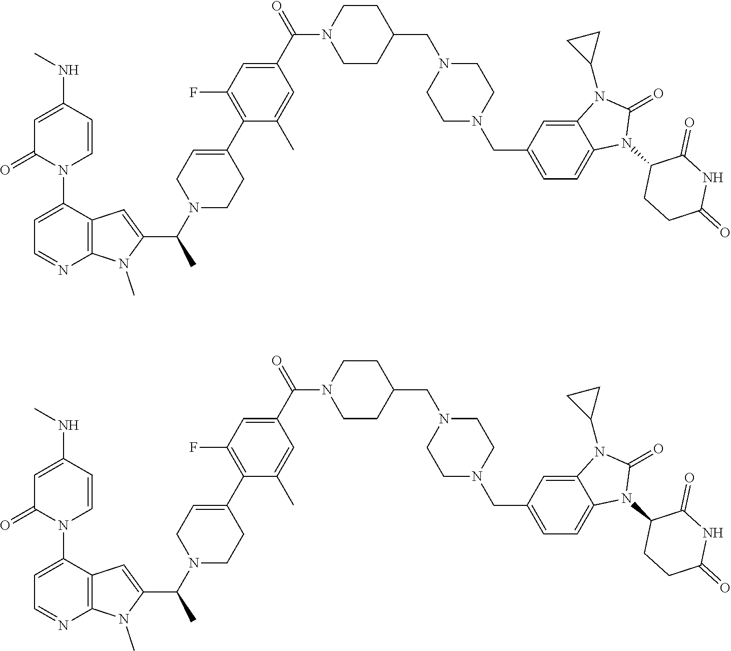 [SMILES: CNc1ccn(-c2ccnc3c2cc([C@H](C)N2CC=C(c4c(C)cc(C(=O)N5CCC(CN6CCN(Cc7ccc8c(c7)n(C7CC7)c(=O)n8[C@H]7CCC(=O)NC7=O)CC6)CC5)cc4F)CC2)n3C)c(=O)c1 ; CNc1ccn(-c2ccnc3c2cc([C@H](C)N2CC=C(c4c(C)cc(C(=O)N5CCC(CN6CCN(Cc7ccc8c(c7)n(C7CC7)c(=O)n8[C@@H]7CCC(=O)NC7=O)CC6)CC5)cc4F)CC2)n3C)c(=O)c1]

### Example 3u123 and 3u124. (S)-3-(3-cyclopropyl-5-((4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)methyl)-2-oxo-2,3-dihydro-1H-benzo[d]imidazol-1-yl)piperidine-2,6-dione and (R)-3-(3-cyclopropyl-5-((4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)methyl)-2-oxo-2,3-dihydro-1H-benzo[d]imidazol-1-yl)piperidine-2,6-dione

Example 3u122 (0.12 g) was resolved by HPLC using a Chiralpak IK 250×30 mm 5 μm column with an eluent of 35% ACN, 35% IPA and 30% DCM (30 mL/min) to give Example 3u123 (25 mg, first peak) and Example 3u124 (26 mg, second peak). The absolute configurations for these compounds at the glutarimide chiral centers were not determined.

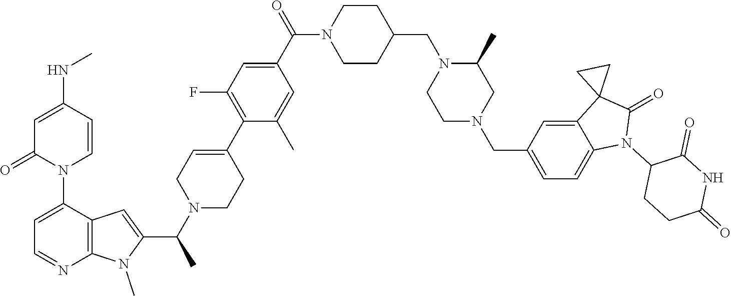 [SMILES: CNc1ccn(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)c(=O)c1]

### Example 3u125. 3-(5′-(((S)-4-((1-(3-fluoro-5-methyl-4-(1-((S)-1-(1-methyl-4-(4-(methylamino)-2-oxopyrimidin-1 (2H)-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-2′-oxospiro[cyclopropane-1,3′-indolin]-1′-yl)piperidine-2,6-dione

Example 3u125 (27 mg) was prepared similarly to Example 3u163 from Ints. 3U45/3C42 (0.10 g/0.15 g).

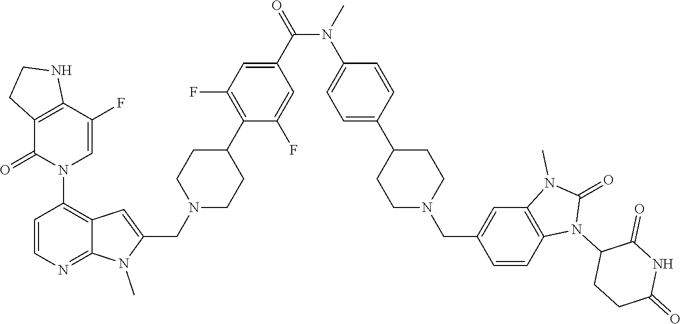 [SMILES: CN(C(=O)c1cc(F)c(C2CCN(Cc3cc4c(-n5cc(F)c6c(c5=O)CCN6)ccnc4n3C)CC2)c(F)c1)c1ccc(C2CCN(Cc3ccc4c(c3)n(C)c(=O)n4C3CCC(=O)NC3=O)CC2)cc1]

### Example 3u126. N-(4-(1-((1-(2,6-dioxopiperidin-3-yl)-3-methyl-2-oxo-2,3-dihydro-1H-benzo[d]imidazol-5-yl)methyl)piperidin-4-yl)phenyl)-3,5-difluoro-4-(1-((4-(7-fluoro-4-oxo-1,2,3,4-tetrahydro-5H-pyrrolo[3,2-c]pyridin-5-yl)-1-methyl-1H-pyrrolo[2,3-b]pyridin-2-yl)methyl)piperidin-4-yl)-N-methylbenzamide

Example 3u126 (0.14 g) was prepared similarly to Example 3u163 from Ints. 3U47/3U69 (0.15 g/0.18 g) using 2-bromo-1-ethyl-pyridinium tetrafluoroborate (1.19 g) instead of T3P.

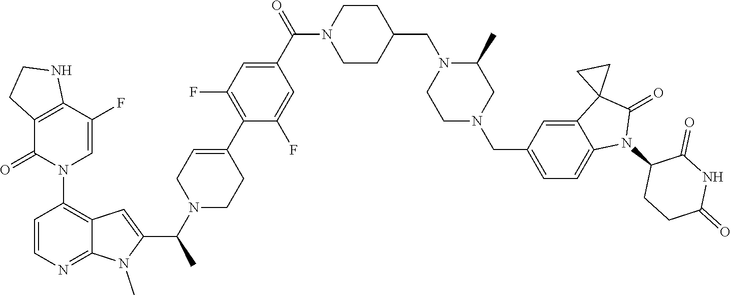 [SMILES: C[C@H]1CN(Cc2ccc3c(c2)C2(CC2)C(=O)N3[C@@H]2CCC(=O)NC2=O)CCN1CC1CCN(C(=O)c2cc(F)c(C3=CCN([C@@H](C)c4cc5c(-n6cc(F)c7c(c6=O)CCN7)ccnc5n4C)CC3)c(F)c2)CC1]

### Example 3u127. (R)-3-(5′-(((S)-4-((1-(3,5-difluoro-4-(1-((S)-1-(4-(7-fluoro-4-oxo-1,2,3,4-tetrahydro-5H-pyrrolo[3,2-c]pyridin-5-yl)-1-methyl-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-2′-oxospiro[cyclopropane-1,3′-indolin]-1′-yl)piperidine-2,6-dione

Example 3u127 (15 mg) was prepared similarly to Example 3u163 from Ints. 2G11/3C42 (90 mg/0.14 g).

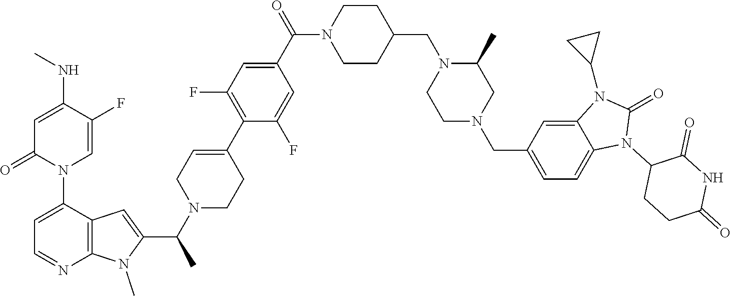 [SMILES: CNc1cc(=O)n(-c2ccnc3c2cc([C@H](C)N2CC=C(c4c(F)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)cc1F]

### Example 3u128. 3-(3-cyclopropyl-5-(((S)-4-((1-(3,5-difluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione

Example 3u128 (0.14 g) was prepared similarly to Example 3u163 from Ints. 2G43/3E78 (0.30 g/0.39 g).

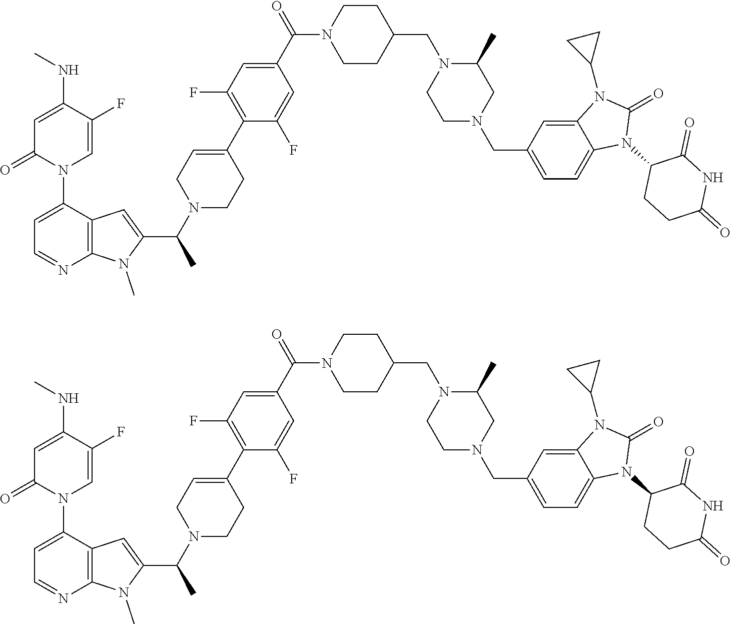 [SMILES: CNc1cc(=O)n(-c2ccnc3c2cc([C@H](C)N2CC=C(c4c(F)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)cc1F ; CNc1cc(=O)n(-c2ccnc3c2cc([C@H](C)N2CC=C(c4c(F)cc(C(=O)N5CCC(CN6CCN(Cc7ccc8c(c7)n(C7CC7)c(=O)n8[C@@H]7CCC(=O)NC7=O)C[C@@H]6C)CC5)cc4F)CC2)n3C)cc1F]

### Example 3u129 and 3u130. (S)-3-(3-cyclopropyl-5-(((S)-4-((1-(3,5-difluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetra-hydropyridin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione and (R)-3-(3-cyclopropyl-5-(((S)-4-((1-(3,5-difluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)-methyl)-2-oxo-2,3-dihydro-1H-benzo[d]imidazol-1-yl)piperidine-2,6-dione

Example 3u128 (0.14 g) was resolved by HPLC using a Chiralpak IK 250×30 mm 5 μm column with an eluent of 35% ACN, 35% IPA and 30% DCM (40 mL/min) to give Example 3u129 (28 mg, first peak) and Example 3u129 (22 mg, second peak). The absolute configurations for these compounds at the glutarimide chiral centers were not determined.

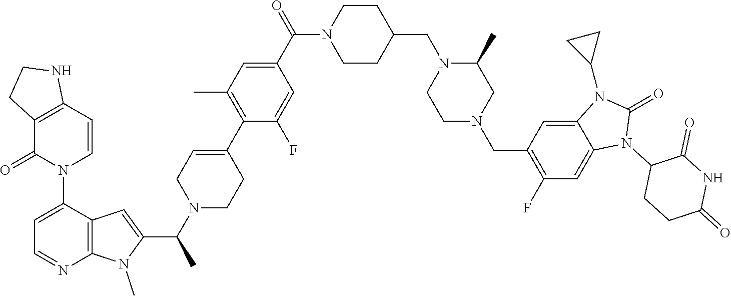 [SMILES: Cc1cc(C(=O)N2CCC(CN3CCN(Cc4cc5c(cc4F)n(C4CCC(=O)NC4=O)c(=O)n5C4CC4)C[C@@H]3C)CC2)cc(F)c1C1=CCN([C@@H](C)c2cc3c(-n4ccc5c(c4=O)CCN5)ccnc3n2C)CC1]

### Example 3u131. 3-(3-cyclopropyl-6-fluoro-5-(((S)-4-((1-(3-fluoro-5-methyl-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione

Example 3u131 (0.18 g) was prepared similarly to Example 3u163 from Ints. 2G53/3E200 (0.25 g/0.41 g).

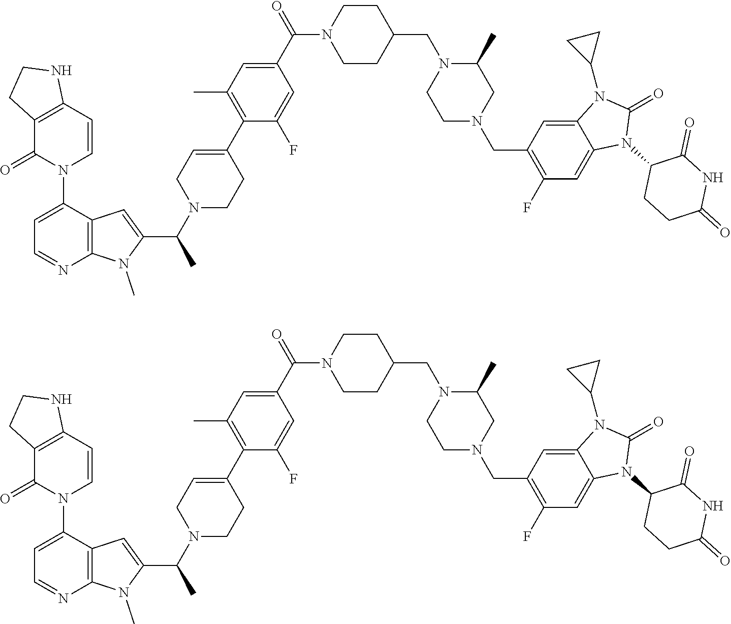 [SMILES: Cc1cc(C(=O)N2CCC(CN3CCN(Cc4cc5c(cc4F)n([C@H]4CCC(=O)NC4=O)c(=O)n5C4CC4)C[C@@H]3C)CC2)cc(F)c1C1=CCN([C@@H](C)c2cc3c(-n4ccc5c(c4=O)CCN5)ccnc3n2C)CC1 ; Cc1cc(C(=O)N2CCC(CN3CCN(Cc4cc5c(cc4F)n([C@@H]4CCC(=O)NC4=O)c(=O)n5C4CC4)C[C@@H]3C)CC2)cc(F)c1C1=CCN([C@@H](C)c2cc3c(-n4ccc5c(c4=O)CCN5)ccnc3n2C)CC1]

### Example 3u132 and 3u133. (S)-3-(3-cyclopropyl-6-fluoro-5-(((S)-4-((1-(3-fluoro-5-methyl-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione and (R)-3-(3-cyclopropyl-6-fluoro-5-(((S)-4-((1-(3-fluoro-5-methyl-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione

Example 3u131 (0.15 g) was resolved by HPLC using a Chiralpak IK 250×30 mm 5 μm column with an eluent of 35% ACN, 35% IPA and 30% DCM (40 mL/min) to give Example 3u132 (25 mg, first peak) and Example 3u133 (25 mg, second peak). The absolute configurations for these compounds at the glutarimide chiral centers were not determined.

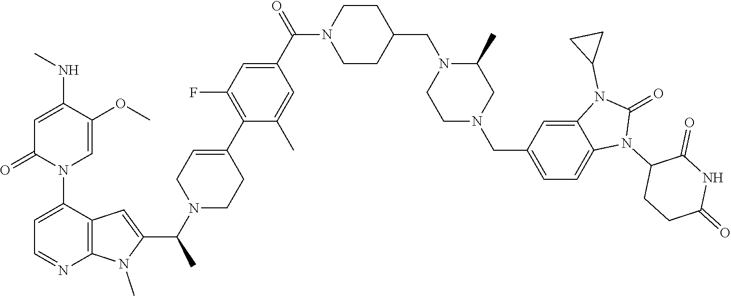 [SMILES: CNc1cc(=O)n(-c2ccnc3c2cc([C@H](C)N2CC=C(c4c(C)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)cc1OC]

### Example 3u134. 3-(3-cyclopropyl-5-(((S)-4-((1-(3-fluoro-4-(1-((S)-1-(4-(5-methoxy-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)-3-methylpiperazin-1-yl)methyl)-2-oxo-2,3-dihydro-1H-benzo[d]imidazol-1-yl)piperidine-2,6-dione

Example 3u134 (53 mg) was prepared similarly to Example 3u163 from Ints. 3U65/3E78 (0.12 g/0.15 g).

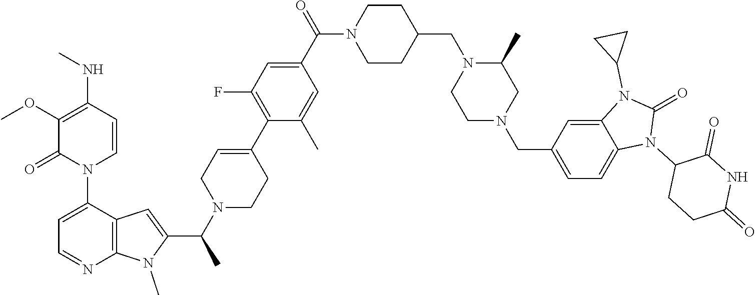 [SMILES: CNc1ccn(-c2ccnc3c2cc([C@H](C)N2CC=C(c4c(C)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)c(=O)c1OC]

### Example 3u135. 3-(3-cyclopropyl-5-(((S)-4-((1-(3-fluoro-4-(1-((S)-1-(4-(3-methoxy-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)-3-methylpiperazin-1-yl)methyl)-2-oxo-2,3-dihydro-1H-benzo[d]imidazol-1-yl)piperidine-2,6-dione

Example 3u135 (40 mg) was prepared similarly to Example 3u163 from Ints. 3U49/3E78 (70 mg/89 mg).

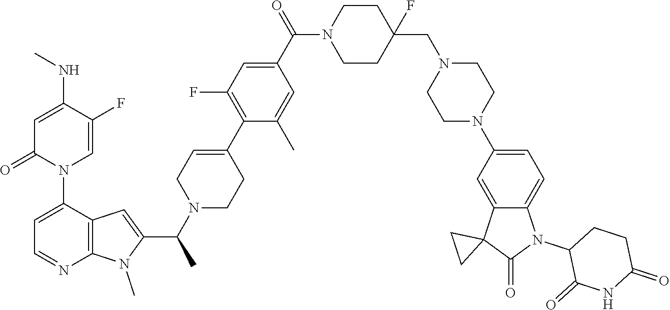 [SMILES: CNc1cc(=O)n(-c2ccnc3c2cc([C@H](C)N2CC=C(c4c(C)cc(C(=O)N5CCC(F)(CN6CCN(c7ccc8c(c7)C7(CC7)C(=O)N8C7CCC(=O)NC7=O)CC6)CC5)cc4F)CC2)n3C)cc1F]

### Example 3u136. 3-(5′-(4-((4-fluoro-1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methyl-benzoyl)piperidin-4-yl)methyl)piperazin-1-yl)-2′-oxospiro[cyclopropane-1,3′-indolin]-1′-yl)piperidine-2,6-dione

Example 3u136 (40 mg) was prepared similarly to Example 3u163 from Ints. 2G45/3U55 (0.13 g/0.11 g).

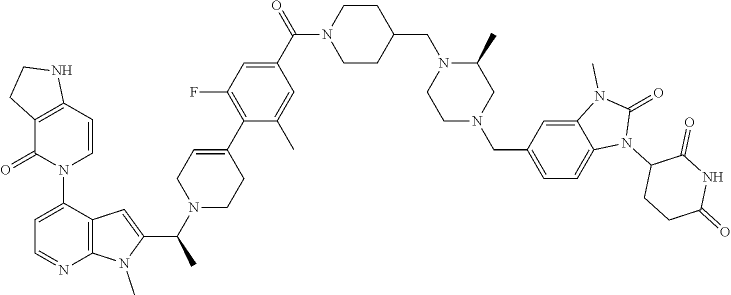 [SMILES: Cc1cc(C(=O)N2CCC(CN3CCN(Cc4ccc5c(c4)n(C)c(=O)n5C4CCC(=O)NC4=O)C[C@@H]3C)CC2)cc(F)c1C1=CCN([C@@H](C)c2cc3c(-n4ccc5c(c4=O)CCN5)ccnc3n2C)CC1]

### Example 3u137. 3-(5-(((S)-4-((1-(3-fluoro-5-methyl-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione

Example 3u137 (85 mg) was prepared similarly to Example 3u163 from Ints. 2G53/3E68 (0.30 g/0.34 g).

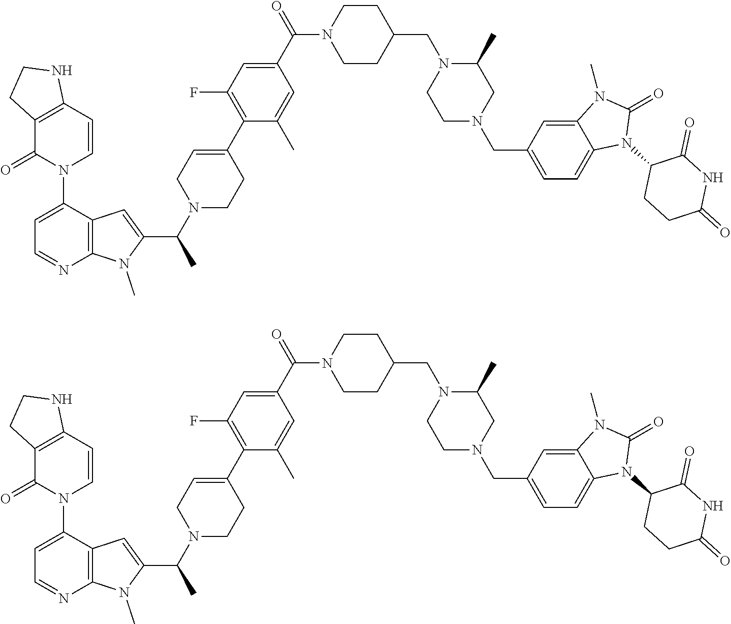 [SMILES: Cc1cc(C(=O)N2CCC(CN3CCN(Cc4ccc5c(c4)n(C)c(=O)n5[C@H]4CCC(=O)NC4=O)C[C@@H]3C)CC2)cc(F)c1C1=CCN([C@@H](C)c2cc3c(-n4ccc5c(c4=O)CCN5)ccnc3n2C)CC1 ; Cc1cc(C(=O)N2CCC(CN3CCN(Cc4ccc5c(c4)n(C)c(=O)n5[C@@H]4CCC(=O)NC4=O)C[C@@H]3C)CC2)cc(F)c1C1=CCN([C@@H](C)c2cc3c(-n4ccc5c(c4=O)CCN5)ccnc3n2C)CC1]

### Example 3u138 and 3u139. (S)-3-(5-(((S)-4-((1-(3-fluoro-5-methyl-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione and (R)-3-(5-(((S)-4-((1-(3-fluoro-5-methyl-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione

Example 3u137 (85 mg) was resolved by HPLC using a Chiralpak IK 250×30 mm 5 μm column with an eluent of 35% ACN, 35% IPA and 30% DCM (38 mL/min) to give Example 3u139 (20 mg, first peak) and Example 3u138 (20 mg, second peak). The absolute configurations for these compounds at the glutarimide chiral centers were not determined.

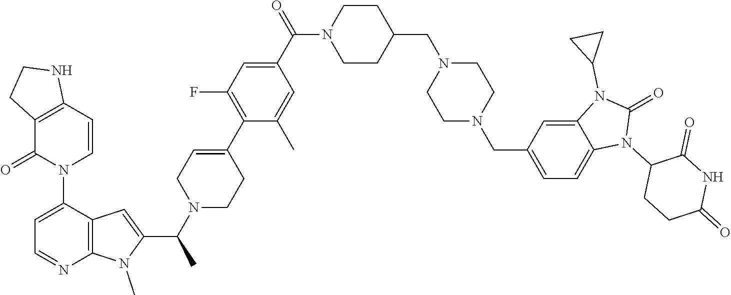 [SMILES: Cc1cc(C(=O)N2CCC(CN3CCN(Cc4ccc5c(c4)n(C4CC4)c(=O)n5C4CCC(=O)NC4=O)CC3)CC2)cc(F)c1C1=CCN([C@@H](C)c2cc3c(-n4ccc5c(c4=O)CCN5)ccnc3n2C)CC1]

### Example 3u140. 3-(3-cyclopropyl-5-((4-((1-(3-fluoro-5-methyl-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)methyl)-2-oxo-2,3-dihydro-1H-benzo[d]imidazol-1-yl)piperidine-2,6-dione

Example 3u140 (70 mg, TFA) was prepared similarly to Example 3u163 from Ints. 2G53/3E33 (0.25 g/0.32 g).

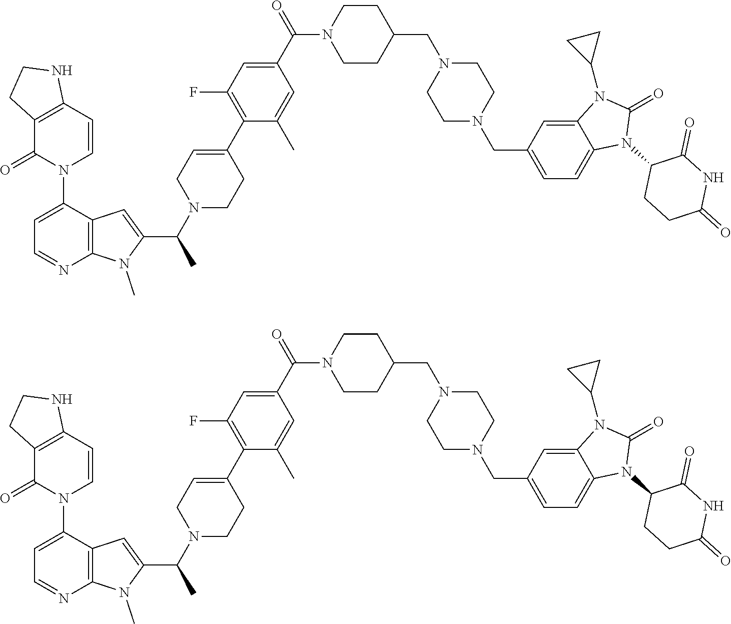 [SMILES: Cc1cc(C(=O)N2CCC(CN3CCN(Cc4ccc5c(c4)n(C4CC4)c(=O)n5[C@H]4CCC(=O)NC4=O)CC3)CC2)cc(F)c1C1=CCN([C@@H](C)c2cc3c(-n4ccc5c(c4=O)CCN5)ccnc3n2C)CC1 ; Cc1cc(C(=O)N2CCC(CN3CCN(Cc4ccc5c(c4)n(C4CC4)c(=O)n5[C@@H]4CCC(=O)NC4=O)CC3)CC2)cc(F)c1C1=CCN([C@@H](C)c2cc3c(-n4ccc5c(c4=O)CCN5)ccnc3n2C)CC1]

### Example 3u141 and 3u142. (S)-3-(3-cyclopropyl-5-((4-((1-(3-fluoro-5-methyl-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)methyl)-2-oxo-2,3-dihydro-1H-benzo[d]imidazol-1-yl)piperidine-2,6-dione and (R)-3-(3-cyclopropyl-5-((4-((1-(3-fluoro-5-methyl-4-(1-((S)-1-(1-methyl-4-(4-oxo-1,2,3,4-tetrahydro-5H-pyrrolo[3,2-c]pyridin-5-yl)-1H-pyrrolo[2,3-b]-pyridin-2-yl)ethyl)-1,2,3,6-tetrahydropyridin-4-yl)benzoyl)piperidin-4-yl)methyl)piperazin-1-yl)-methyl)-2-oxo-2,3-dihydro-1H-benzo[d]imidazol-1-yl)piperidine-2,6-dione

Example 3u140 (57 mg) was resolved by HPLC using a Chiralpak IK 250×30 mm 5 μm column with an eluent of 35% ACN, 35% IPA and 30% DCM (4 mL/min) to give Example 3u141 (6 mg, first peak) and Example 3u142 (13 mg, second peak). The absolute configurations for these compounds at the glutarimide chiral centers were not determined.

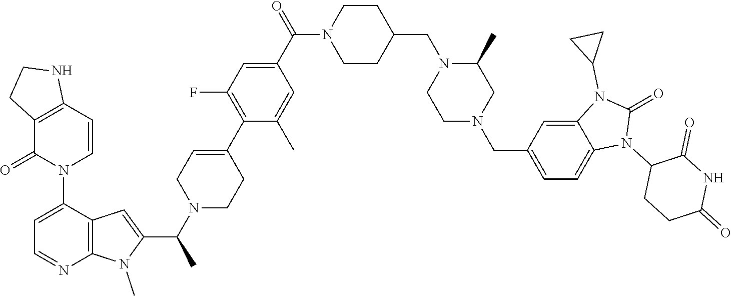 [SMILES: Cc1cc(C(=O)N2CCC(CN3CCN(Cc4ccc5c(c4)n(C4CC4)c(=O)n5C4CCC(=O)NC4=O)C[C@@H]3C)CC2)cc(F)c1C1=CCN([C@@H](C)c2cc3c(-n4ccc5c(c4=O)CCN5)ccnc3n2C)CC1]

### Example 3u143. 3-(3-cyclopropyl-5-(((S)-4-((1-(3-fluoro-5-methyl-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione

Example 3u143 (0.12 g, TFA salt) was prepared similarly to Example 3u163 from Ints. 2G53/3E78 (0.20 g/0.30 g).

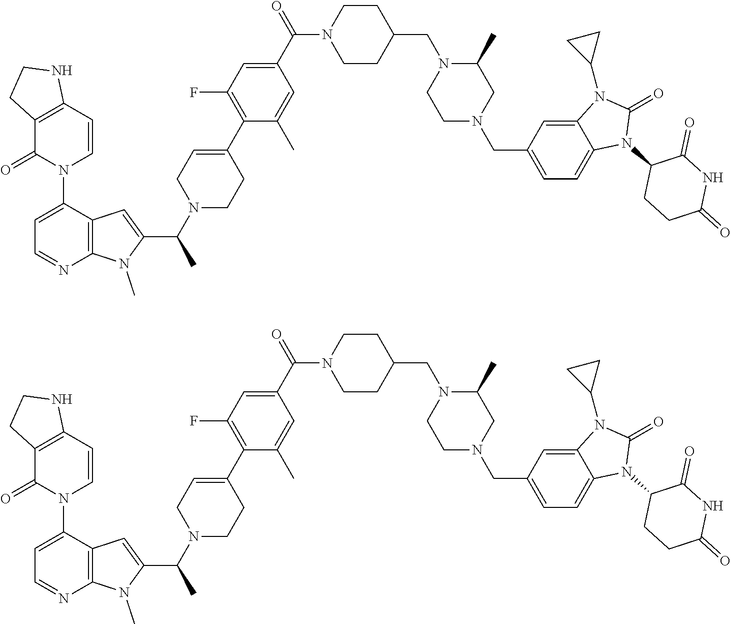 [SMILES: Cc1cc(C(=O)N2CCC(CN3CCN(Cc4ccc5c(c4)n(C4CC4)c(=O)n5[C@@H]4CCC(=O)NC4=O)C[C@@H]3C)CC2)cc(F)c1C1=CCN([C@@H](C)c2cc3c(-n4ccc5c(c4=O)CCN5)ccnc3n2C)CC1 ; Cc1cc(C(=O)N2CCC(CN3CCN(Cc4ccc5c(c4)n(C4CC4)c(=O)n5[C@H]4CCC(=O)NC4=O)C[C@@H]3C)CC2)cc(F)c1C1=CCN([C@@H](C)c2cc3c(-n4ccc5c(c4=O)CCN5)ccnc3n2C)CC1]

### Example 3u144 and 3u145. (R)-3-(3-cyclopropyl-5-(((S)-4-((1-(3-fluoro-5-methyl-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione and (S)-3-(3-cyclopropyl-5-(((S)-4-((1-(3-fluoro-5-methyl-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione

Example 3u143 (95 mg) was resolved by HPLC using a Chiralpak IK 250×30 mm 5 μm column with an eluent of 35% ACN, 35% IPA and 30% DCM (42 mL/min) to give Example 3u144 (32 mg, first peak) and Example 3u145 (18 mg, second peak). The absolute configurations for these compounds at the glutarimide chiral centers were not determined.

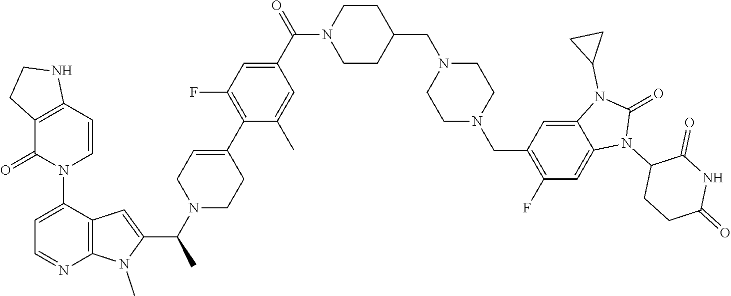 [SMILES: Cc1cc(C(=O)N2CCC(CN3CCN(Cc4cc5c(cc4F)n(C4CCC(=O)NC4=O)c(=O)n5C4CC4)CC3)CC2)cc(F)c1C1=CCN([C@@H](C)c2cc3c(-n4ccc5c(c4=O)CCN5)ccnc3n2C)CC1]

### Example 3u146. 3-(3-cyclopropyl-6-fluoro-5-((4-((1-(3-fluoro-5-methyl-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)methyl)-2-oxo-2,3-dihydro-1H-benzo[d]imidazol-1-yl)piperidine-2,6-dione

Example 3u146 (95 mg, TFA) was prepared similarly to Example 3u163 from Ints. 2G53/3E200 (0.20 g/0.23 g).

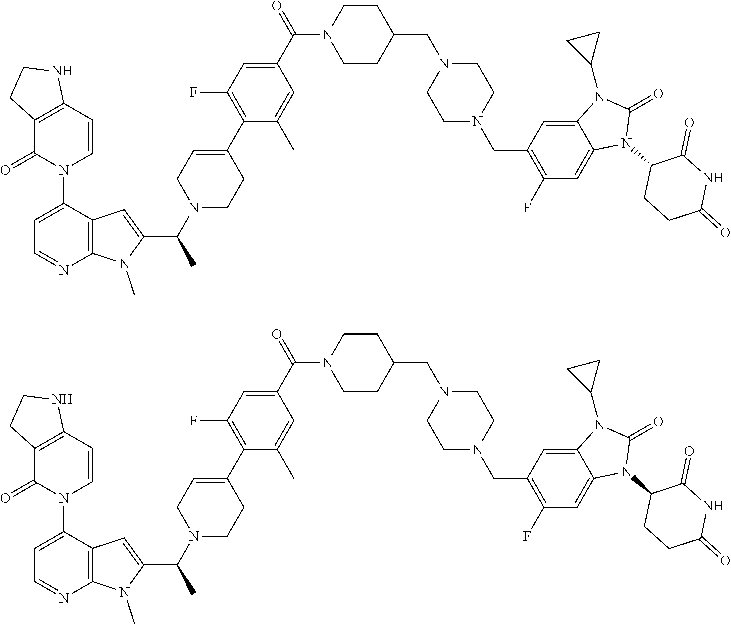 [SMILES: Cc1cc(C(=O)N2CCC(CN3CCN(Cc4cc5c(cc4F)n([C@H]4CCC(=O)NC4=O)c(=O)n5C4CC4)CC3)CC2)cc(F)c1C1=CCN([C@@H](C)c2cc3c(-n4ccc5c(c4=O)CCN5)ccnc3n2C)CC1 ; Cc1cc(C(=O)N2CCC(CN3CCN(Cc4cc5c(cc4F)n([C@@H]4CCC(=O)NC4=O)c(=O)n5C4CC4)CC3)CC2)cc(F)c1C1=CCN([C@@H](C)c2cc3c(-n4ccc5c(c4=O)CCN5)ccnc3n2C)CC1]

### Example 3u147 and 3u148. (S)-3-(3-cyclopropyl-6-fluoro-5-((4-((1-(3-fluoro-5-methyl-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)methyl)-2-oxo-2,3-dihydro-1H-benzo[d]imidazol-1-yl)piperidine-2,6-dione and (R)-3-(3-cyclopropyl-6-fluoro-5-((4-((1-(3-fluoro-5-methyl-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)methyl)-2-oxo-2,3-dihydro-1H-benzo[d]imidazol-1-yl)piperidine-2,6-dione

Example 3u146 (40 mg) was resolved by HPLC using a Chiralpak IK 250×10 mm 5 μm column with an eluent of 35% ACN, 35% IPA and 30% DCM (5 mL/min) to give Example 3u147 (11 mg, first peak) and Example 3u148 (13 mg, second peak). The absolute configurations for these compounds at the glutarimide chiral centers were not determined.

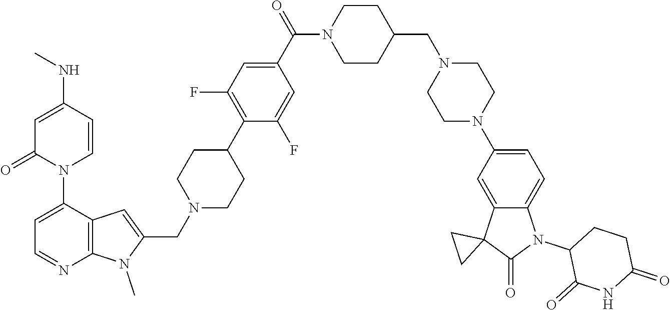 [SMILES: CNc1ccn(-c2ccnc3c2cc(CN2CCC(c4c(F)cc(C(=O)N5CCC(CN6CCN(c7ccc8c(c7)C7(CC7)C(=O)N8C7CCC(=O)NC7=O)CC6)CC5)cc4F)CC2)n3C)c(=O)c1]

### Example 3u149. 3-(5′-(4-((1-(3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)methyl)piperazin-1-yl)-2′-oxospiro[cyclopropane-1,3′-indolin]-1′-yl)piperidine-2,6-dione

Example 3u149 (0.32 g) was prepared similarly to Example 3u163 from Ints. 2G108/3U1 (0.30 g/0.43 g).

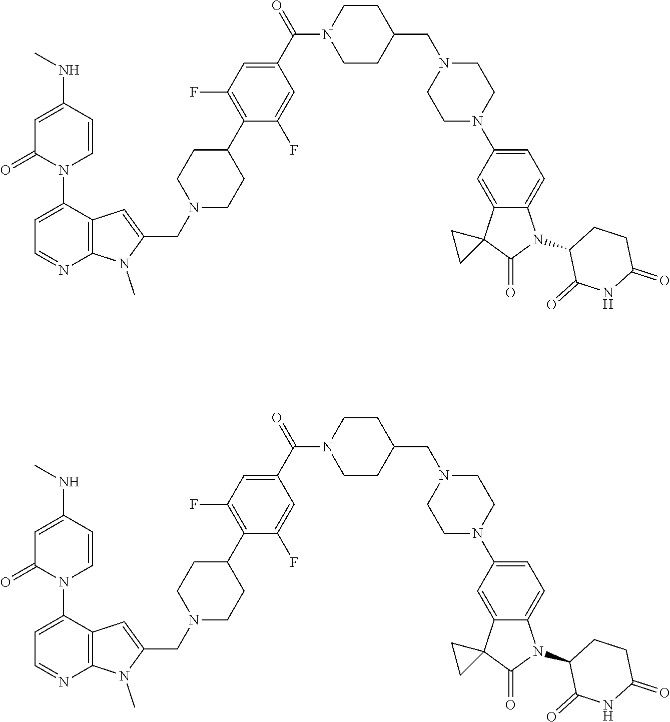 [SMILES: CNc1ccn(-c2ccnc3c2cc(CN2CCC(c4c(F)cc(C(=O)N5CCC(CN6CCN(c7ccc8c(c7)C7(CC7)C(=O)N8[C@@H]7CCC(=O)NC7=O)CC6)CC5)cc4F)CC2)n3C)c(=O)c1 ; CNc1ccn(-c2ccnc3c2cc(CN2CCC(c4c(F)cc(C(=O)N5CCC(CN6CCN(c7ccc8c(c7)C7(CC7)C(=O)N8[C@H]7CCC(=O)NC7=O)CC6)CC5)cc4F)CC2)n3C)c(=O)c1]

### Example 3u150 and 3u151. (R)-3-(5′-(4-((1-(3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)-methyl)piperazin-1-yl)-2′-oxospiro[cyclopropane-1,3′-indolin]-1′-yl)piperidine-2,6-dione and (S)-3-(5′-(4-((1-(3,5-difluoro-4-(1-((1-methyl-4-(4-(methylamino)-2-oxopyridin-1 (2H)-yl)-1H-pyrrolo[2,3-b]-pyridin-2-yl)methyl)piperidin-4-yl)benzoyl)piperidin-4-yl)methyl)piperazin-1-yl)-2′-oxospiro-[cyclopropane-1,3′-indolin]-1′-yl)piperidine-2,6-dione

Example 3u149 (0.31 g) was resolved by HPLC using a Chiralpak IK 250×30 mm 5 μm column with an eluent of 35% ACN, 35% IPA and 30% DCM (26 mL/min) to give Example 3u151 (57 mg, first peak) and Example 3u150 (6 mg, second peak). The second peak required a second resolution using a Chiralpak IK 250×10 mm 5 μm column with an eluent of 35% ACN, 35% IPA and 30% DCM (6 mL/min). The absolute configurations for these compounds at the glutarimide chiral centers were not determined.

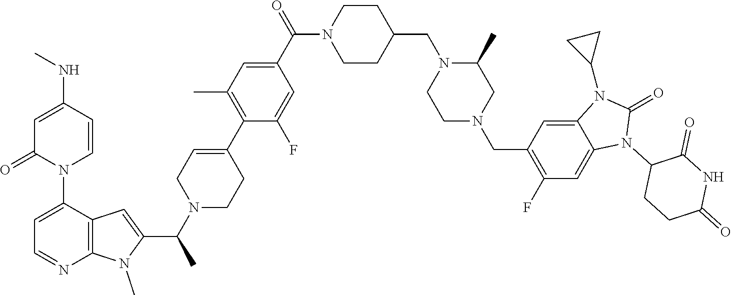 [SMILES: CNc1ccn(-c2ccnc3c2cc([C@H](C)N2CC=C(c4c(C)cc(C(=O)N5CCC(CN6CCN(Cc7cc8c(cc7F)n(C7CCC(=O)NC7=O)c(=O)n8C7CC7)C[C@@H]6C)CC5)cc4F)CC2)n3C)c(=O)c1]

### Example 3u166. 3-(3-cyclopropyl-6-fluoro-5-(((S)-4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione

Ints. 2G46/3E200 (0.40 g/0.30 g) were stirred 16 h in DMF/Et₃N (5:1, 2.4 mL) and T3P (50% in EtOAc, 0.48 mL) at 0° C. to RT. The reaction mixture diluted with water to precipitate a solid that was HPLC-purified to give Example 3u166 (140 mg).

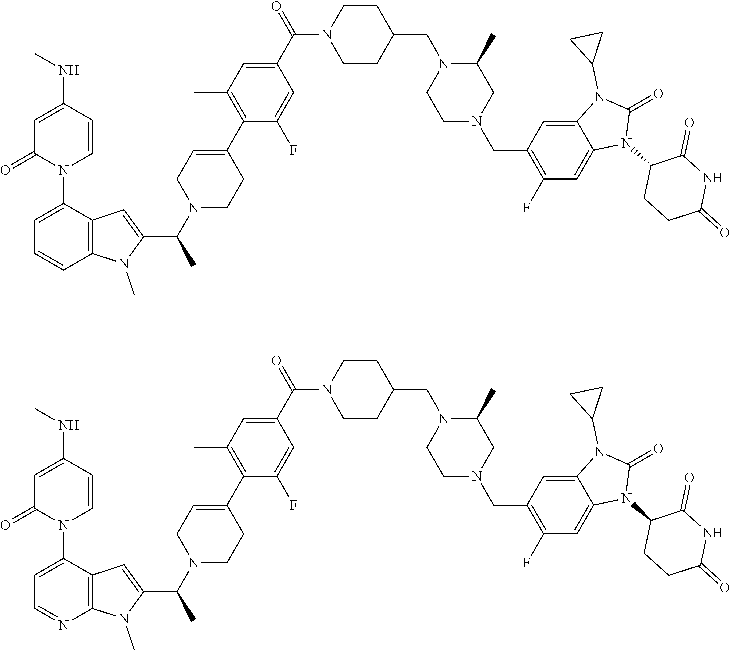 [SMILES: CNc1ccn(-c2cccc3c2cc([C@H](C)N2CC=C(c4c(C)cc(C(=O)N5CCC(CN6CCN(Cc7cc8c(cc7F)n([C@H]7CCC(=O)NC7=O)c(=O)n8C7CC7)C[C@@H]6C)CC5)cc4F)CC2)n3C)c(=O)c1 ; CNc1ccn(-c2ccnc3c2cc([C@H](C)N2CC=C(c4c(C)cc(C(=O)N5CCC(CN6CCN(Cc7cc8c(cc7F)n([C@@H]7CCC(=O)NC7=O)c(=O)n8C7CC7)C[C@@H]6C)CC5)cc4F)CC2)n3C)c(=O)c1]

Example 3u167 and 3u168. (S)-3-(3-cyclopropyl-6-fluoro-5-(((S)-4-((1-(3-fluoro-5-methyl-4-(1-((S)-1-(1-methyl-4-(4-(methylamino)-2-oxopyridin-1 (2H)-yl)-1H-indol-2-yl)ethyl)-1,2,3,6-tetrahydropyridin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione and (R)-3-(3-cyclopropyl-6-fluoro-5-(((S)-4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)-methyl)-2-oxo-2,3-dihydro-1H-benzo[d]imidazol-1-yl)piperidine-2,6-dione

Example 3u166 (140 mg) was resolved by HPLC using a Chiralpak IK 30×250 mm 5 μm column with an eluent of 35% ACN, 35% IPA and 30% DCM (30 mL/min) to give Example 3u167 (55 mg, first peak) and Example 3u168 (50 mg, second peak). The absolute configurations for these compounds at the glutarimide chiral centers were not determined.

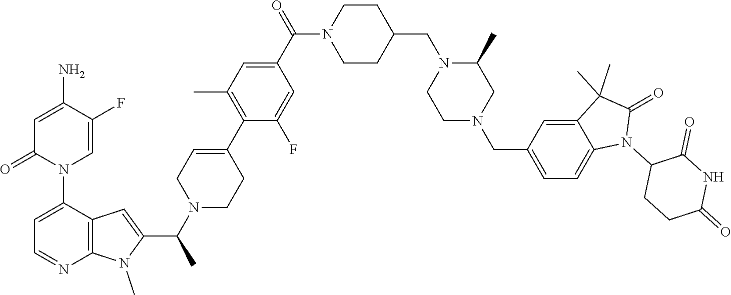 [SMILES: Cc1cc(C(=O)N2CCC(CN3CCN(Cc4ccc5c(c4)C(C)(C)C(=O)N5C4CCC(=O)NC4=O)C[C@@H]3C)CC2)cc(F)c1C1=CCN([C@@H](C)c2cc3c(-n4cc(F)c(N)cc4=O)ccnc3n2C)CC1]

### Example 3u169. 3-(5-(((S)-4-((1-(4-(1-((S)-1-(4-(4-amino-5-fluoro-2-oxopyridin-1 (2H)-yl)-1-methyl-1H-pyrrolo[2,3-b]pyridin-2-yl)ethyl)-1,2,3,6-tetrahydropyridin-4-yl)-3-fluoro-5-methylbenzoyl)piperidin-4-yl)methyl)-3-methylpiperazin-1-yl)methyl)-3,3-dimethyl-2-oxoindolin-1-yl)piperidine-2,6-dione

Example 3u169 (30 mg) was prepared similarly to Example 3u166 from Ints. 3U127/3E291 (0.15 g/0.21 g).

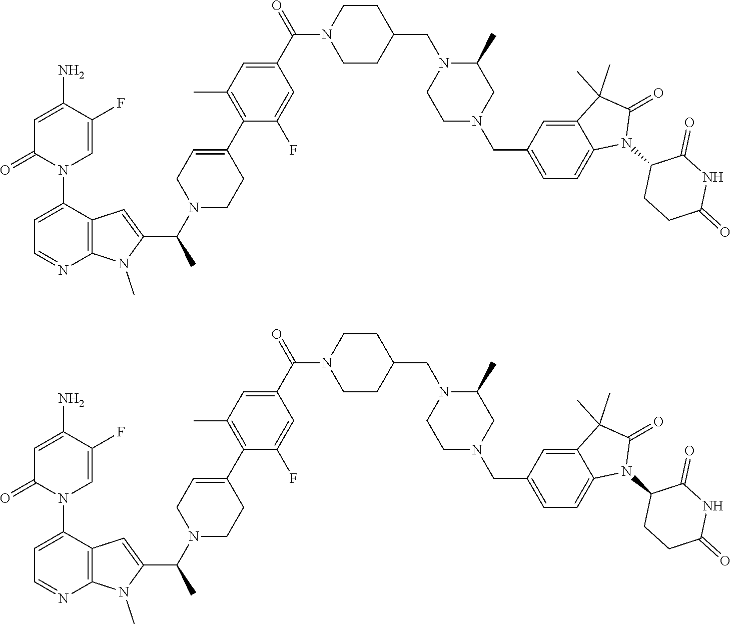 [SMILES: Cc1cc(C(=O)N2CCC(CN3CCN(Cc4ccc5c(c4)C(C)(C)C(=O)N5[C@H]4CCC(=O)NC4=O)C[C@@H]3C)CC2)cc(F)c1C1=CCN([C@@H](C)c2cc3c(-n4cc(F)c(N)cc4=O)ccnc3n2C)CC1 ; Cc1cc(C(=O)N2CCC(CN3CCN(Cc4ccc5c(c4)C(C)(C)C(=O)N5[C@@H]4CCC(=O)NC4=O)C[C@@H]3C)CC2)cc(F)c1C1=CCN([C@@H](C)c2cc3c(-n4cc(F)c(N)cc4=O)ccnc3n2C)CC1]

### Example 3u170 and 3u171. (S)-3-(5-(((S)-4-((1-(4-(1-((S)-1-(4-(4-amino-5-fluoro-2-oxopyridin-1 (2H)-yl)-1-methyl-1H-pyrrolo[2,3-b]pyridin-2-yl)ethyl)-1,2,3,6-tetrahydropyridin-4-yl)-3-fluoro-5-methylbenzoyl)piperidin-4-yl)methyl)-3-methylpiperazin-1-yl)methyl)-3,3-dimethyl-2-oxoindolin-1-yl)piperidine-2,6-dione and (R)-3-(5-(((S)-4-((1-(4-(1-((S)-1-(4-(4-amino-5-fluoro-2-oxopyridin-1 (2H)-yl)-1-methyl-1H-pyrrolo[2,3-b]pyridin-2-yl)ethyl)-1,2,3,6-tetrahydropyridin-4-yl)-3-fluoro-5-methylbenzoyl)piperidin-4-yl)methyl)-3-methylpiperazin-1-yl)methyl)-3,3-dimethyl-2-oxoindolin-1-yl)piperidine-2,6-dione

Example 3u169 (30 mg) was resolved by HPLC using a Chiralpak IK 10×250 mm 5 μm column with an eluent of 35% ACN, 35% IPA and 30% DCM (3 mL/min) to give Example 3u170 (7 mg, first peak) and Example 3u171 (8 mg, second peak). The absolute configurations for these compounds at the glutarimide chiral centers were not determined.

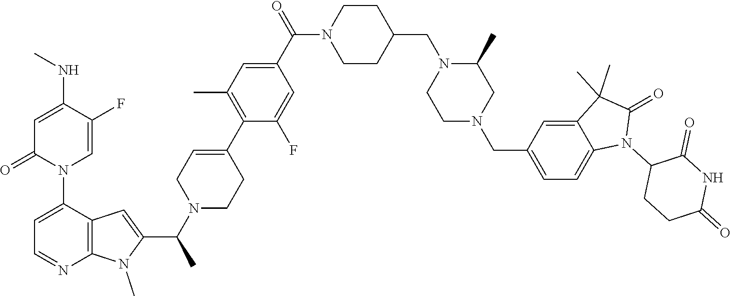 [SMILES: CNc1cc(=O)n(-c2ccnc3c2cc([C@H](C)N2CC=C(c4c(C)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)cc1F]

### Example 3u172. 3-(5-(((S)-4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)-3-methylpiperazin-1-yl)methyl)-3,3-dimethyl-2-oxoindolin-1-yl)piperidine-2,6-dione

Example 3u172 (80 mg) was prepared similarly to Example 3u166 from Ints. 2G45/3E291 (0.30 g/0.38 g).

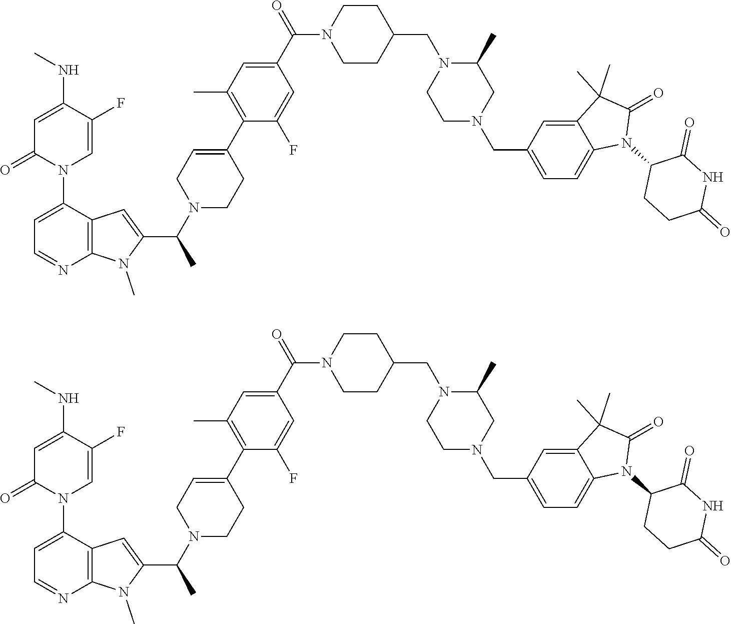 [SMILES: CNc1cc(=O)n(-c2ccnc3c2cc([C@H](C)N2CC=C(c4c(C)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)cc1F ; CNc1cc(=O)n(-c2ccnc3c2cc([C@H](C)N2CC=C(c4c(C)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)cc1F]

### Example 3u173 and 3u174. (S)-3-(5-(((S)-4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)-3-methylpiperazin-1-yl)methyl)-3,3-dimethyl-2-oxoindolin-1-yl)piperidine-2,6-dione and (R)-3-(5-(((S)-4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)-3-methylpiperazin-1-yl)methyl)-3,3-dimethyl-2-oxoindolin-1-yl)piperidine-2,6-dione

Example 3u172 (65 mg) was resolved by HPLC using a Chiralpak IK 10×250 mm 5 μm column with an eluent of 35% ACN, 35% IPA and 30% DCM (3 mL/min) to give Example 3u173 (10 mg, first peak) and Example 3u174 (19 mg, second peak). The absolute configurations for these compounds at the glutarimide chiral centers were not determined.

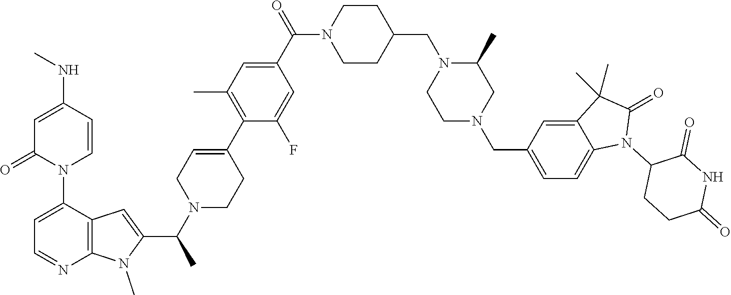 [SMILES: CNc1ccn(-c2ccnc3c2cc([C@H](C)N2CC=C(c4c(C)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)c(=O)c1]

### Example 3u175. 3-(5-(((S)-4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)-piperidin-4-yl)methyl)-3-methylpiperazin-1-yl)methyl)-3,3-dimethyl-2-oxoindolin-1-yl)piperidine-2,6-dione

Example 3u175 (0.12 g) was prepared similarly to Example 3u166 from Ints. 2G46/3E291 (0.25 g/0.43 g).

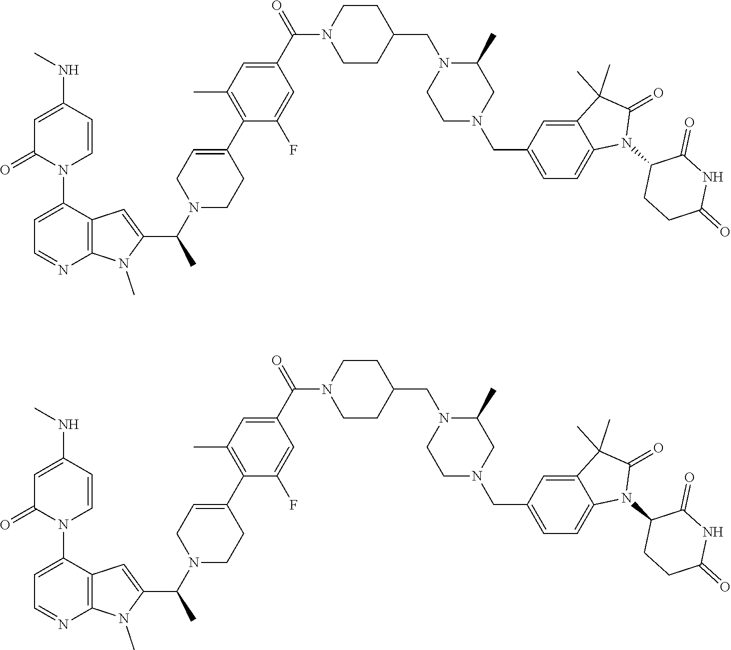 [SMILES: CNc1ccn(-c2ccnc3c2cc([C@H](C)N2CC=C(c4c(C)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)c(=O)c1 ; CNc1ccn(-c2ccnc3c2cc([C@H](C)N2CC=C(c4c(C)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)c(=O)c1]

### Example 3u176 and 3u177. (S)-3-(5-(((S)-4-((1-(3-fluoro-5-methyl-4-(1-((S)-1-(1-methyl-4-(4-(methyl-amino)-2-oxopyridin-1 (2H)-yl)-1H-pyrrolo[2,3-b]pyridin-2-yl)ethyl)-1,2,3,6-tetrahydropyridin-4-yl)-benzoyl)piperidin-4-yl)methyl)-3-methylpiperazin-1-yl)methyl)-3,3-dimethyl-2-oxoindolin-1-yl)-piperidine-2,6-dione and (R)-3-(5-(((S)-4-((1-(3-fluoro-5-methyl-4-(1-((S)-1-(1-methyl-4-(4-(methyl-amino)-2-oxopyridin-1 (2H)-yl)-1H-pyrrolo[2,3-b]pyridin-2-yl)ethyl)-1,2,3,6-tetrahydropyridin-4-yl)-benzoyl)piperidin-4-yl)methyl)-3-methylpiperazin-1-yl)methyl)-3,3-dimethyl-2-oxoindolin-1-yl)-piperidine-2,6-dione

Example 3u175 (100 mg) was resolved by HPLC using a Chiralpak IK 10×250 mm 5 μm column with an eluent of 35% ACN, 35% IPA and 30% DCM (3 mL/min) to give Example 3u176 (15 mg, first peak) and Example 3u177 (18 mg, second peak). The absolute configurations for these compounds at the glutarimide chiral centers were not determined.

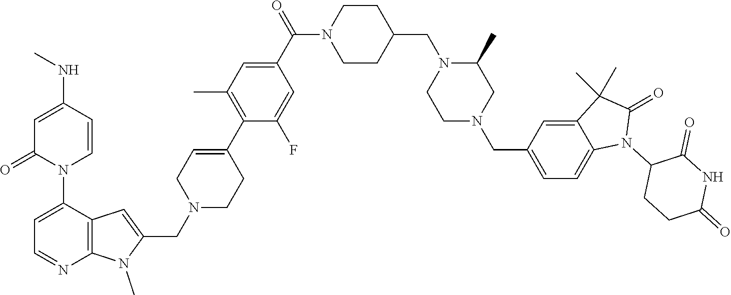 [SMILES: CNc1ccn(-c2ccnc3c2cc(CN2CC=C(c4c(C)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)c(=O)c1]

### Example 3u178. 3-(5-(((S)-4-((1-(3-fluoro-5-methyl-4-(1-((1-methyl-4-(4-(methylamino)-2-oxopyridin-1 (2H)-yl)-1H-pyrrolo[2,3-b]pyridin-2-yl)methyl)-1,2,3,6-tetrahydropyridin-4-yl)benzoyl)piperidin-4-yl)methyl)-3-methylpiperazin-1-yl)methyl)-3,3-dimethyl-2-oxoindolin-1-yl)piperidine-2,6-dione

Example 3u178 (0.14 g) was prepared similarly to Example 3u166 from Ints. 1M60/3E291 (0.30 g/0.50 g).

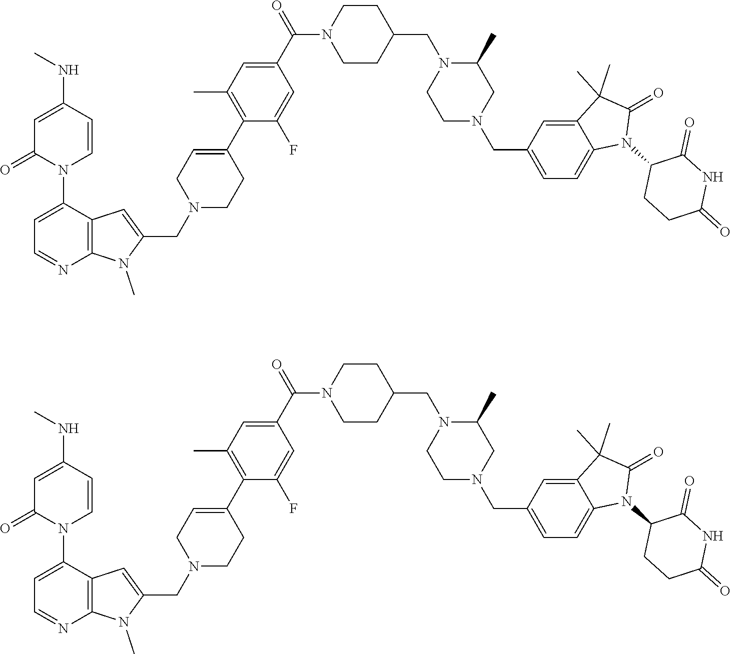 [SMILES: CNc1ccn(-c2ccnc3c2cc(CN2CC=C(c4c(C)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)c(=O)c1 ; CNc1ccn(-c2ccnc3c2cc(CN2CC=C(c4c(C)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)c(=O)c1]

### Example 3u179 and 3u180. (S)-3-(5-(((S)-4-((1-(3-fluoro-5-methyl-4-(1-((1-methyl-4-(4-(methylamino)-2-oxopyridin-1 (2H)-yl)-1H-pyrrolo[2,3-b]pyridin-2-yl)methyl)-1,2,3,6-tetrahydropyridin-4-yl)benzoyl)piperidin-4-yl)methyl)-3-methylpiperazin-1-yl)methyl)-3,3-dimethyl-2-oxoindolin-1-yl)piperidine-2,6-dione and (R)-3-(5-(((S)-4-((1-(3-fluoro-5-methyl-4-(1-((1-methyl-4-(4-(methylamino)-2-oxopyridin-1 (2H)-yl)-1H-pyrrolo[2,3-b]pyridin-2-yl)methyl)-1,2,3,6-tetrahydropyridin-4-yl)benzoyl)piperidin-4-yl)methyl)-3-methylpiperazin-1-yl)methyl)-3,3-dimethyl-2-oxoindolin-1-yl)piperidine-2,6-dione

Example 3u178 (130 mg) was resolved by HPLC using a CELLULOSE-SC 10×250 mm 5 μm column with an eluent of 40% ACN, 40% IPA and 20% DCM (28 mL/min) to give Example 3u179 (42 mg, first peak) and Example 3u180 (55 mg, second peak). The absolute configurations for these compounds at the glutarimide chiral centers were not determined.

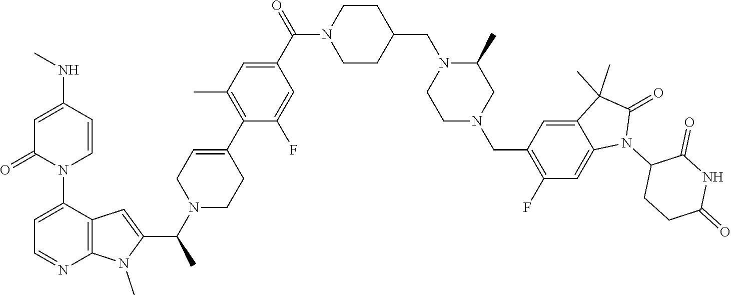 [SMILES: CNc1ccn(-c2ccnc3c2cc([C@H](C)N2CC=C(c4c(C)cc(C(=O)N5CCC(CN6CCN(Cc7cc8c(cc7F)N(C7CCC(=O)NC7=O)C(=O)C8(C)C)C[C@@H]6C)CC5)cc4F)CC2)n3C)c(=O)c1]

### Example 3u181. 3-(6-fluoro-5-(((S)-4-((1-(3-fluoro-5-methyl-4-(1-((S)-1-(1-methyl-4-(4-(methyl-amino)-2-oxopyridin-1 (2H)-yl)-1H-pyrrolo[2,3-b]pyridin-2-yl)ethyl)-1,2,3,6-tetrahydropyridin-4-yl)-benzoyl)piperidin-4-yl)methyl)-3-methylpiperazin-1-yl)methyl)-3,3-dimethyl-2-oxoindolin-1-yl)-piperidine-2,6-dione

Example 3u181 (80 mg) was prepared similarly to Example 3u166 from Ints. 2G46/3C47 (0.30 g/0.37 g).

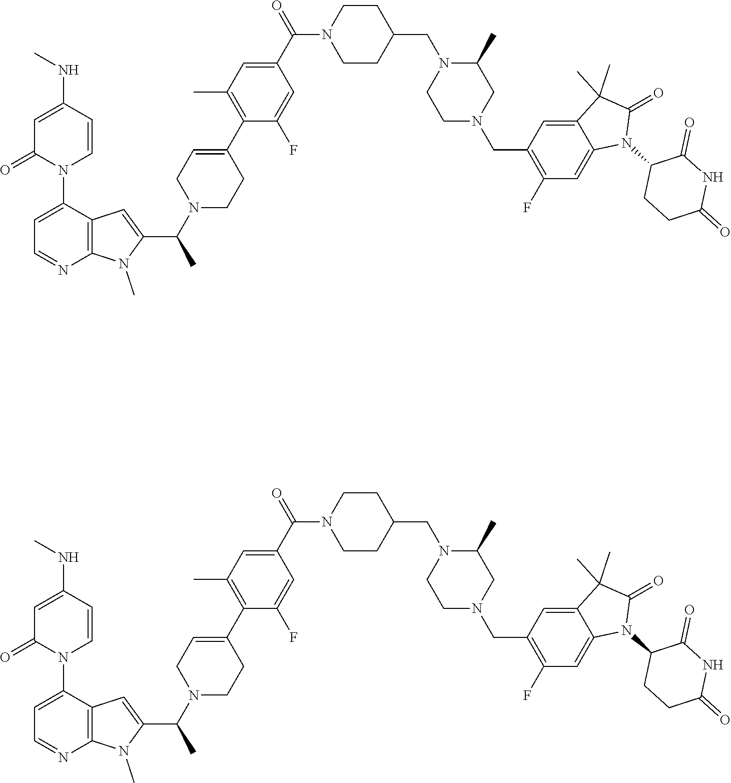 [SMILES: CNc1ccn(-c2ccnc3c2cc([C@H](C)N2CC=C(c4c(C)cc(C(=O)N5CCC(CN6CCN(Cc7cc8c(cc7F)N([C@H]7CCC(=O)NC7=O)C(=O)C8(C)C)C[C@@H]6C)CC5)cc4F)CC2)n3C)c(=O)c1 ; CNc1ccn(-c2ccnc3c2cc([C@H](C)N2CC=C(c4c(C)cc(C(=O)N5CCC(CN6CCN(Cc7cc8c(cc7F)N([C@@H]7CCC(=O)NC7=O)C(=O)C8(C)C)C[C@@H]6C)CC5)cc4F)CC2)n3C)c(=O)c1]

### Example 3u182 and 3u183. (S)-3-(6-fluoro-5-(((S)-4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-3,3-dimethyl-2-oxoindolin-1-yl)piperidine-2,6-dione and (R)-3-(6-fluoro-5-(((S)-4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-3,3-dimethyl-2-oxoindolin-1-yl)piperidine-2,6-dione

Example 3u181 (60 mg) was resolved by HPLC using a Chiralpak IK 10×250 mm 5 μm column with an eluent of 35% ACN, 35% IPA and 30% DCM (3 mL/min) to give Example 3u182 (19 mg, first peak) and Example 3u183 (20 mg, second peak). The absolute configurations for these compounds at the glutarimide chiral centers were not determined.

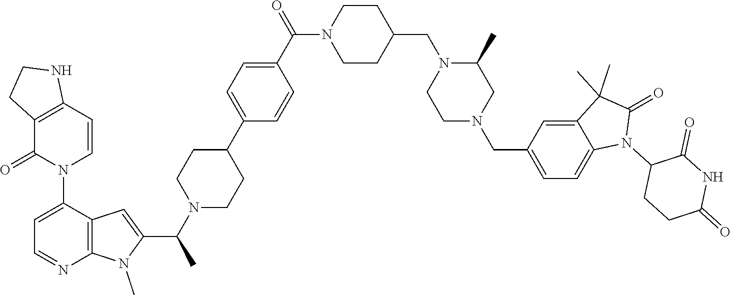 [SMILES: C[C@H]1CN(Cc2ccc3c(c2)C(C)(C)C(=O)N3C2CCC(=O)NC2=O)CCN1CC1CCN(C(=O)c2ccc(C3CCN([C@@H](C)c4cc5c(-n6ccc7c(c6=O)CCN7)ccnc5n4C)CC3)cc2)CC1]

### Example 3u184. 3-(3,3-dimethyl-5-(((S)-3-methyl-4-((1-(4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)piperidin-4-yl)benzoyl)-piperidin-4-yl)methyl)piperazin-1-yl)methyl)-2-oxoindolin-1-yl)piperidine-2,6-dione

Example 3u184 (90 mg) was prepared similarly to Example 3u166 from Ints. 3E178/3E291 (0.30 g/0.37 g).

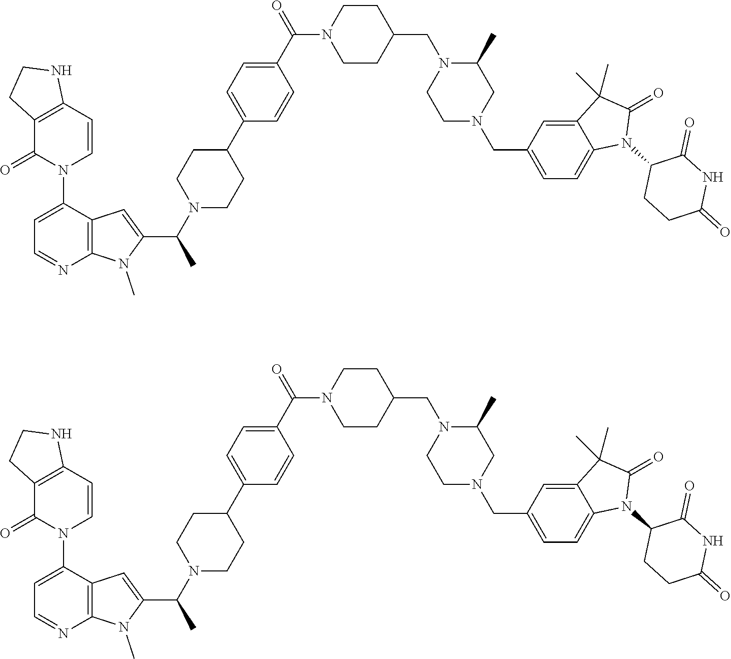 [SMILES: C[C@H]1CN(Cc2ccc3c(c2)C(C)(C)C(=O)N3[C@H]2CCC(=O)NC2=O)CCN1CC1CCN(C(=O)c2ccc(C3CCN([C@@H](C)c4cc5c(-n6ccc7c(c6=O)CCN7)ccnc5n4C)CC3)cc2)CC1 ; C[C@H]1CN(Cc2ccc3c(c2)C(C)(C)C(=O)N3[C@@H]2CCC(=O)NC2=O)CCN1CC1CCN(C(=O)c2ccc(C3CCN([C@@H](C)c4cc5c(-n6ccc7c(c6=O)CCN7)ccnc5n4C)CC3)cc2)CC1]

Example 3u185 and 3u186. (S)-3-(3,3-dimethyl-5-(((S)-3-methyl-4-((1-(4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)piperidin-4-yl)benzoyl)piperidin-4-yl)methyl)piperazin-1-yl)methyl)-2-oxoindolin-1-yl)piperidine-2,6-dione and (R)-3-(3,3-dimethyl-5-(((S)-3-methyl-4-((1-(4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)piperidin-4-yl)benzoyl)piperidin-4-yl)methyl)piperazin-1-yl)methyl)-2-oxoindolin-1-yl)piperidine-2,6-dione

Example 3u184 (70 mg) was resolved by HPLC using a Chiralpak IK 10×250 mm 5 μm column with an eluent of 35% ACN, 35% IPA and 30% DCM (4 mL/min) to give Example 3u185 (25 mg, first peak) and Example 3u186 (24 mg, second peak). The absolute configurations for these compounds at the glutarimide chiral centers were not determined.

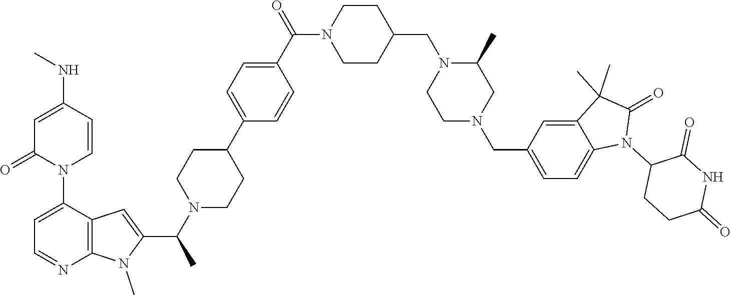 [SMILES: CNc1ccn(-c2ccnc3c2cc([C@H](C)N2CCC(c4ccc(C(=O)N5CCC(CN6CCN(Cc7ccc8c(c7)C(C)(C)C(=O)N8C7CCC(=O)NC7=O)C[C@@H]6C)CC5)cc4)CC2)n3C)c(=O)c1]

Example 3u187. 3-(3,3-dimethyl-5-(((S)-3-methyl-4-((1-(4-(1-((S)-1-(1-methyl-4-(4-(methylamino)-2-oxopyridin-1 (2H)-yl)-1H-pyrrolo[2,3-b]pyridin-2-yl)ethyl)piperidin-4-yl)benzoyl)piperidin-4-yl)-methyl)piperazin-1-yl)methyl)-2-oxoindolin-1-yl)piperidine-2,6-dione

Example 3u184 (90 mg) was prepared similarly to Example 3u166 from Ints. 3E171/3E291 (0.25 g/0.43 g).

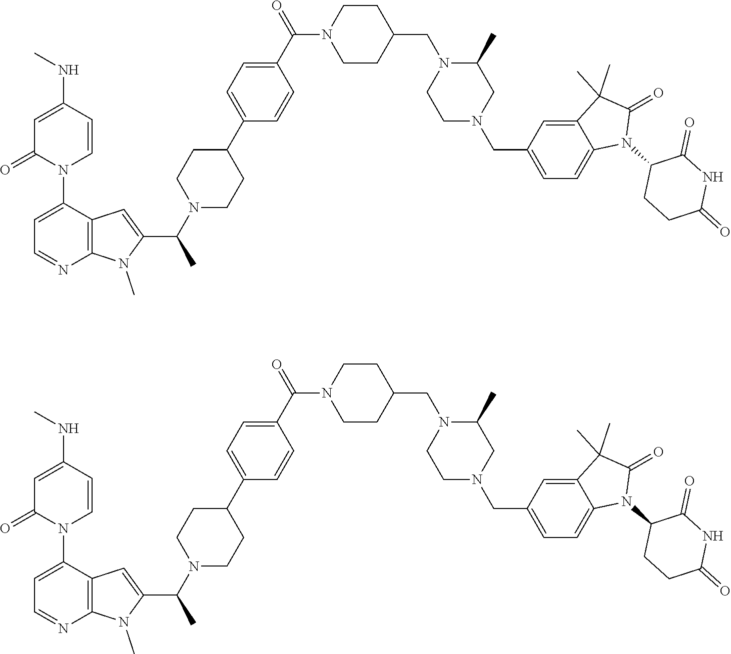 [SMILES: CNc1ccn(-c2ccnc3c2cc([C@H](C)N2CCC(c4ccc(C(=O)N5CCC(CN6CCN(Cc7ccc8c(c7)C(C)(C)C(=O)N8[C@H]7CCC(=O)NC7=O)C[C@@H]6C)CC5)cc4)CC2)n3C)c(=O)c1 ; CNc1ccn(-c2ccnc3c2cc([C@H](C)N2CCC(c4ccc(C(=O)N5CCC(CN6CCN(Cc7ccc8c(c7)C(C)(C)C(=O)N8[C@@H]7CCC(=O)NC7=O)C[C@@H]6C)CC5)cc4)CC2)n3C)c(=O)c1]

Example 3u188 and 3u189. (S)-3-(3,3-dimethyl-5-(((S)-3-methyl-4-((1-(4-(1-((S)-1-(1-methyl-4-(4-(methylamino)-2-oxopyridin-1 (2H)-yl)-1H-pyrrolo[2,3-b]pyridin-2-yl)ethyl)piperidin-4-yl)benzoyl)-piperidin-4-yl)methyl)piperazin-1-yl)methyl)-2-oxoindolin-1-yl)piperidine-2,6-dione and (R)-3-(3,3-dimethyl-5-(((S)-3-methyl-4-((1-(4-(1-((S)-1-(1-methyl-4-(4-(methylamino)-2-oxopyridin-1 (2H)-yl)-1H-pyrrolo[2,3-b]pyridin-2-yl)ethyl)piperidin-4-yl)benzoyl)piperidin-4-yl)methyl)piperazin-1-yl)methyl)-2-oxoindolin-1-yl)piperidine-2,6-dione

Example 3u187 (91 mg) was resolved by HPLC using a Chiralpak IK 10×250 mm 5 μm column with an eluent of 35% ACN, 35% IPA and 30% DCM (3 mL/min) to give Example 3u188 (28 mg, first peak) and Example 3u189 (32 mg, second peak). The absolute configurations for these compounds at the glutarimide chiral centers were not determined.

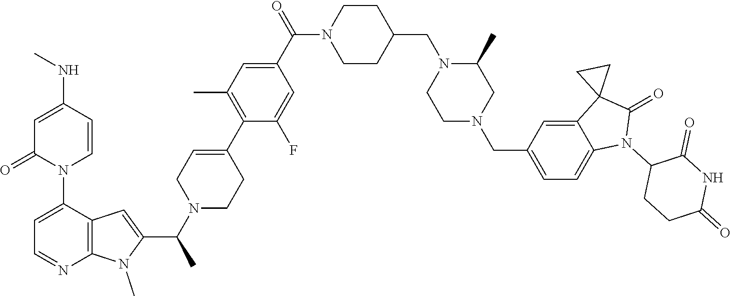 [SMILES: CNc1ccn(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)c(=O)c1]

Example 3u190. 3-(5′-(((S)-4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-2′-oxospiro[cyclopropane-1,3′-indolin]-1′-yl)piperidine-2,6-dione

Example 3u184 (90 mg) was prepared similarly to Example 3u166 from Ints. 2G46/3C42 (0.28 g/0.57 g).

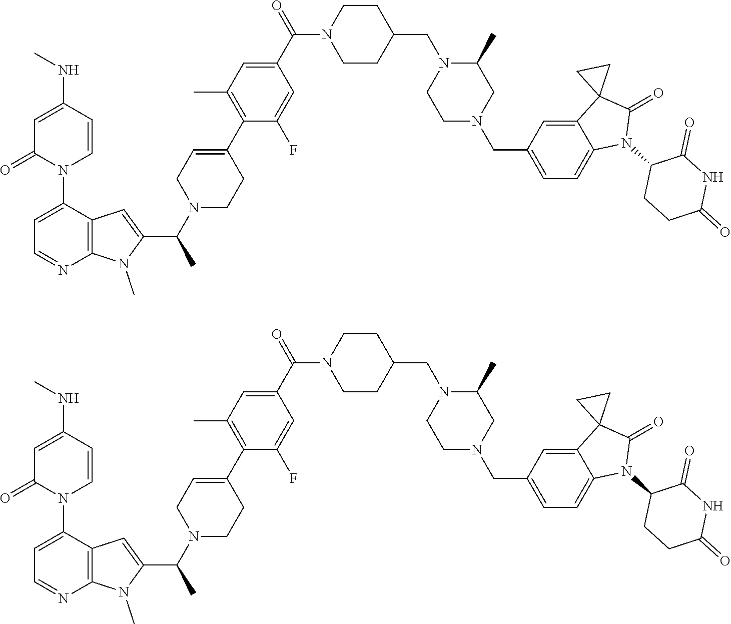 [SMILES: CNc1ccn(-c2ccnc3c2cc([C@H](C)N2CC=C(c4c(C)cc(C(=O)N5CCC(CN6CCN(Cc7ccc8c(c7)C7(CC7)C(=O)N8[C@H]7CCC(=O)NC7=O)C[C@@H]6C)CC5)cc4F)CC2)n3C)c(=O)c1 ; CNc1ccn(-c2ccnc3c2cc([C@H](C)N2CC=C(c4c(C)cc(C(=O)N5CCC(CN6CCN(Cc7ccc8c(c7)C7(CC7)C(=O)N8[C@@H]7CCC(=O)NC7=O)C[C@@H]6C)CC5)cc4F)CC2)n3C)c(=O)c1]

Example 3u191 and 3u192. (S)-3-(5′-(((S)-4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-2′-oxospiro[cyclopropane-1,3′-indolin]-1′-yl)piperidine-2,6-dione and (R)-3-(5′-(((S)-4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydro-pyridin-4-yl)benzoyl)piperidin-4-yl)methyl)-3-methylpiperazin-1-yl)methyl)-2′-oxospiro-[cyclopropane-1,3′-indolin]-1′-yl)piperidine-2,6-dione

Example 3u190 (114 mg) was resolved by HPLC using a Chiralpak IK 30×150 mm 5 μm column with an eluent of 35% ACN, 35% IPA and 30% DCM (28 mL/min) to give Example 3u191 (35 mg, first peak) and Example 3u192 (35 mg, second peak). The absolute configurations for these compounds at the glutarimide chiral centers were not determined.

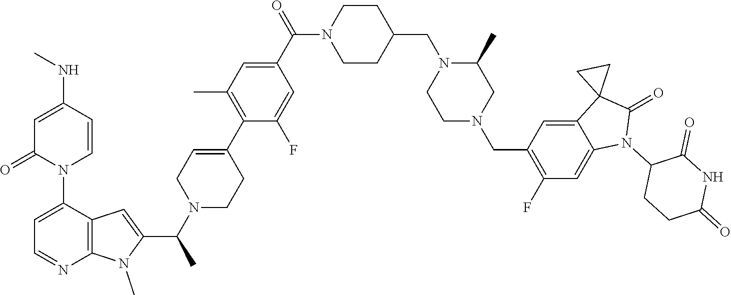 [SMILES: CNc1ccn(-c2ccnc3c2cc([C@H](C)N2CC=C(c4c(C)cc(C(=O)N5CCC(CN6CCN(Cc7cc8c(cc7F)N(C7CCC(=O)NC7=O)C(=O)C87CC7)C[C@@H]6C)CC5)cc4F)CC2)n3C)c(=O)c1]

### Example 3u193. 3-(6′-fluoro-5′-(((S)-4-((1-(3-fluoro-5-methyl-4-(1-((S)-1-(1-methyl-4-(4-(methyl-amino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-2′-oxospiro[cyclopropane-1,3′-indolin]-1′-yl)piperidine-2,6-dione

Example 3u193 (155 mg) was prepared similarly to Example 3u190 from Ints. 2G46/3U129 (0.28 g/0.41 g).

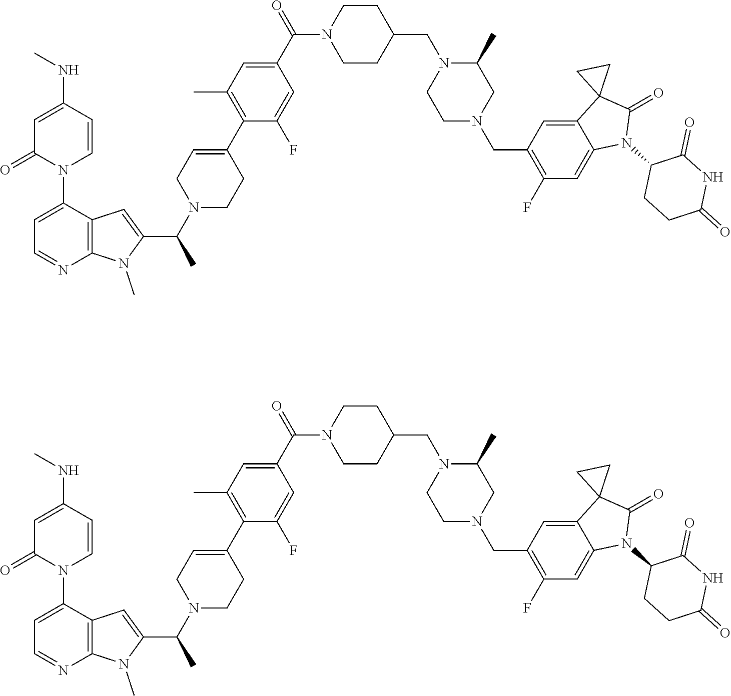 [SMILES: CNc1ccn(-c2ccnc3c2cc([C@H](C)N2CC=C(c4c(C)cc(C(=O)N5CCC(CN6CCN(Cc7cc8c(cc7F)N([C@H]7CCC(=O)NC7=O)C(=O)C87CC7)C[C@@H]6C)CC5)cc4F)CC2)n3C)c(=O)c1 ; CNc1ccn(-c2ccnc3c2cc([C@H](C)N2CC=C(c4c(C)cc(C(=O)N5CCC(CN6CCN(Cc7cc8c(cc7F)N([C@@H]7CCC(=O)NC7=O)C(=O)C87CC7)C[C@@H]6C)CC5)cc4F)CC2)n3C)c(=O)c1]

### Example 3u194 and 3u195. (S)-3-(6′-fluoro-5′-(((S)-4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-2′-oxospiro[cyclopropane-1,3′-indolin]-1′-yl)piperidine-2,6-dione and (R)-3-(6′-fluoro-5′-(((S)-4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-2′-oxospiro[cyclopropane-1,3′-indolin]-1′-yl)piperidine-2,6-dione

Example 3u193 (115 mg) was resolved by HPLC using a Chiralpak IK 10×250 mm 5 μm column with an eluent of 35% ACN, 35% IPA and 30% DCM (5 mL/min) to give Example 3u194 (40 mg, first peak) and Example 3u195 (40 mg, second peak). The absolute configurations for these compounds at the glutarimide chiral centers were not determined.

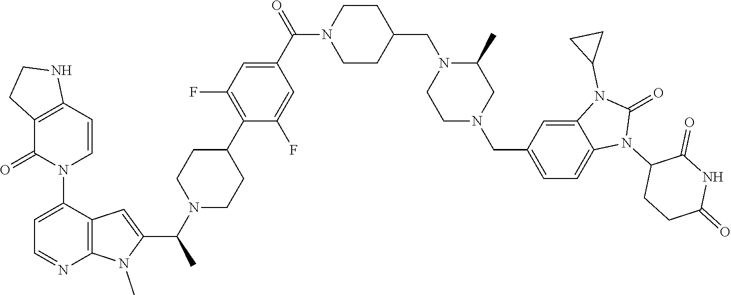 [SMILES: C[C@H]1CN(Cc2ccc3c(c2)n(C2CC2)c(=O)n3C2CCC(=O)NC2=O)CCN1CC1CCN(C(=O)c2cc(F)c(C3CCN([C@@H](C)c4cc5c(-n6ccc7c(c6=O)CCN7)ccnc5n4C)CC3)c(F)c2)CC1]

### Example 3u196 3-(3-cyclopropyl-5-(((S)-4-((1-(3,5-difluoro-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)piperidin-4-yl)benzoyl)-piperidin-4-yl)methyl)-3-methylpiperazin-1-yl)methyl)-2-oxo-2,3-dihydro-1H-benzo[d]imidazol-1-yl)-piperidine-2,6-dione

Example 3u196 (90 mg) was prepared similarly to Example 3u166 from Ints. 3U131/3E78 (0.45 g/0.54 g).

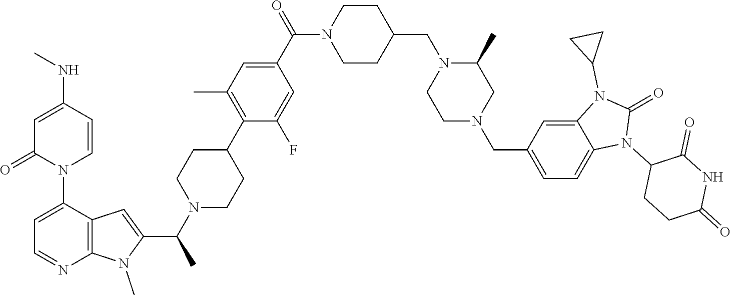 [SMILES: CNc1ccn(-c2ccnc3c2cc([C@H](C)N2CCC(c4c(C)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)c(=O)c1]

### Example 3u197. 3-(3-cyclopropyl-5-(((S)-4-((1-(3-fluoro-5-methyl-4-(1-((S)-1-(1-methyl-4-(4-(methyl-amino)-2-oxopyridin-1 (2H)-yl)-1H-pyrrolo[2,3-b]pyridin-2-yl)ethyl)piperidin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione

Example 3u184 (90 mg) was prepared similarly to Example 3u166 from Ints. 2G54/3C42 (0.30 g/0.40 g).

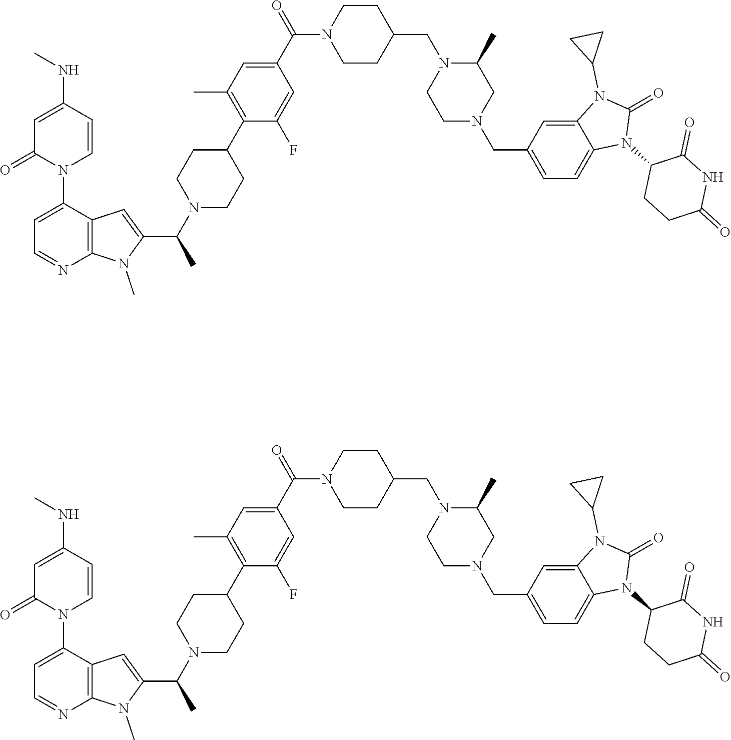 [SMILES: CNc1ccn(-c2ccnc3c2cc([C@H](C)N2CCC(c4c(C)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)c(=O)c1 ; CNc1ccn(-c2ccnc3c2cc([C@H](C)N2CCC(c4c(C)cc(C(=O)N5CCC(CN6CCN(Cc7ccc8c(c7)n(C7CC7)c(=O)n8[C@@H]7CCC(=O)NC7=O)C[C@@H]6C)CC5)cc4F)CC2)n3C)c(=O)c1]

### Example 3u198 and 3u199. (S)-3-(3-cyclopropyl-5-(((S)-4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)piperidin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione and (R)-3-(3-cyclopropyl-5-(((S)-4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)piperidin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione

Example 3u197 (70 mg) was resolved by HPLC using a Chiralpak IK 10×250 mm 5 μm column with an eluent of 35% ACN, 35% IPA and 30% DCM (5 mL/min) to give Example 3u198 (22 mg, first peak) and Example 3u199 (19 mg, second peak). The absolute configurations for these compounds at the glutarimide chiral centers were not determined.

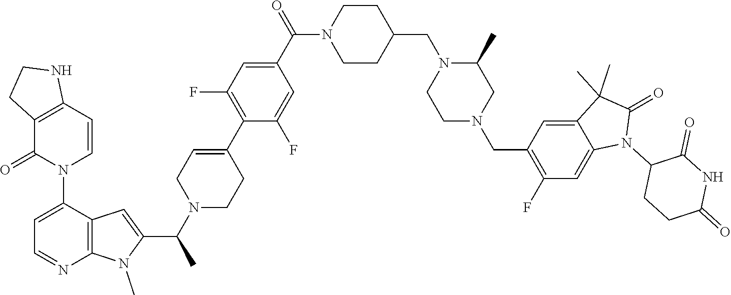 [SMILES: C[C@H]1CN(Cc2cc3c(cc2F)N(C2CCC(=O)NC2=O)C(=O)C3(C)C)CCN1CC1CCN(C(=O)c2cc(F)c(C3=CCN([C@@H](C)c4cc5c(-n6ccc7c(c6=O)CCN7)ccnc5n4C)CC3)c(F)c2)CC1]

### Example 3u200. 3-(5-(((S)-4-((1-(3,5-difluoro-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)-benzoyl)piperidin-4-yl)methyl)-3-methylpiperazin-1-yl)methyl)-6-fluoro-3,3-dimethyl-2-oxoindolin-1-yl)piperidine-2,6-dione

Example 3u200 (90 mg) was prepared similarly to Example 3u166 from Ints. 3U122/3C47 (0.20 g/0.18 g).

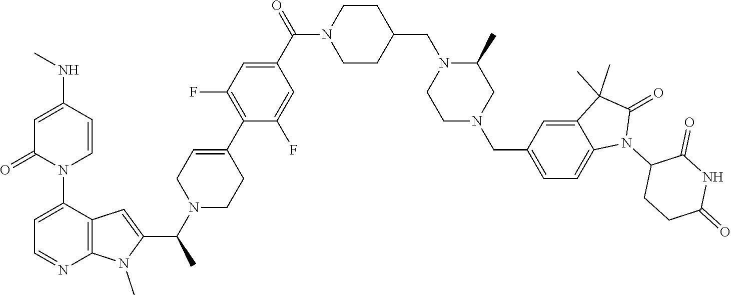 [SMILES: CNc1ccn(-c2ccnc3c2cc([C@H](C)N2CC=C(c4c(F)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)c(=O)c1]

### Example 3u201. 3-(5-(((S)-4-((1-(3,5-difluoro-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)-methyl)-3-methylpiperazin-1-yl)methyl)-3,3-dimethyl-2-oxoindolin-1-yl)piperidine-2,6-dione

Example 3u201 (130 mg) was prepared similarly to Example 3u166 from Ints. 2G118/3E291 (0.30 g/0.45 g).

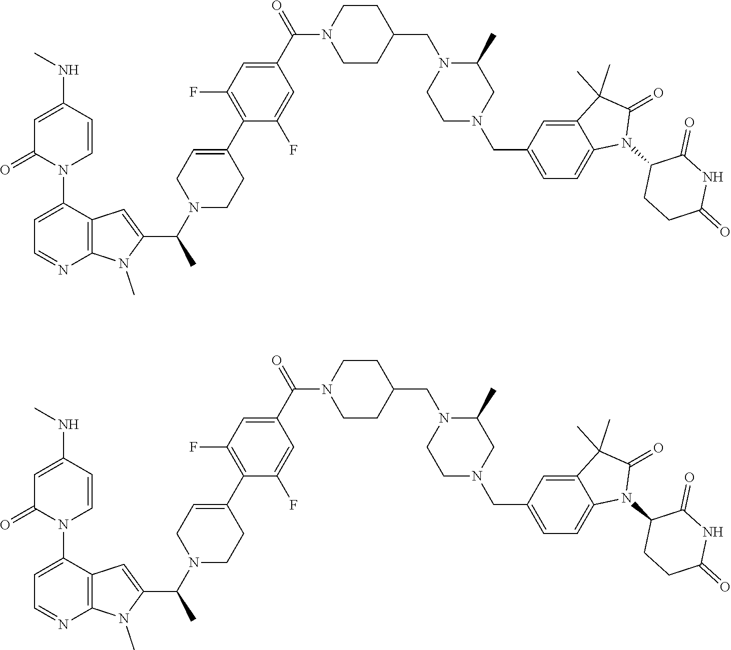 [SMILES: CNc1ccn(-c2ccnc3c2cc([C@H](C)N2CC=C(c4c(F)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)c(=O)c1 ; CNc1ccn(-c2ccnc3c2cc([C@H](C)N2CC=C(c4c(F)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)c(=O)c1]

### Example 3u202 and 3u203. (S)-3-(5-(((S)-4-((1-(3,5-difluoro-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-3,3-dimethyl-2-oxoindolin-1-yl)piperidine-2,6-dione and (R)-3-(5-(((S)-4-((1-(3,5-difluoro-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-3,3-dimethyl-2-oxoindolin-1-yl)piperidine-2,6-dione

Example 3u201 (125 mg) was resolved by HPLC using a Chiralpak IK 30×250 mm 5 μm column with an eluent of 35% ACN, 35% IPA and 30% DCM (28 mL/min) to give Example 3u202 (50 mg, first peak) and Example 3u203 (43 mg, second peak). The absolute configurations for these compounds at the glutarimide chiral centers were not determined.

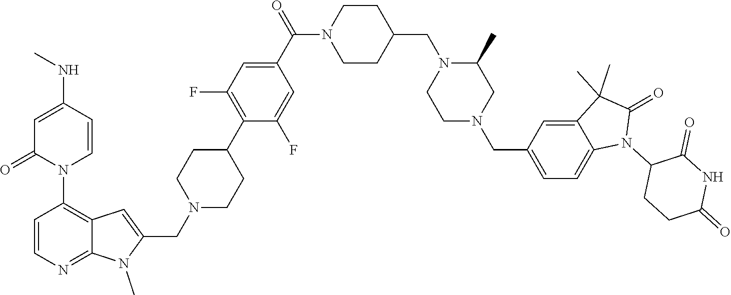 [SMILES: CNc1ccn(-c2ccnc3c2cc(CN2CCC(c4c(F)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)c(=O)c1]

### Example 3u204. 3-(5-(((S)-4-((1-(3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)methyl)-3-methyl-piperazin-1-yl)methyl)-3,3-dimethyl-2-oxoindolin-1-yl)piperidine-2,6-dione

Example 3u204 (110 mg) was prepared similarly to Example 3u201 from Ints. 2G108/3E291 (0.30 g/0.49 g).

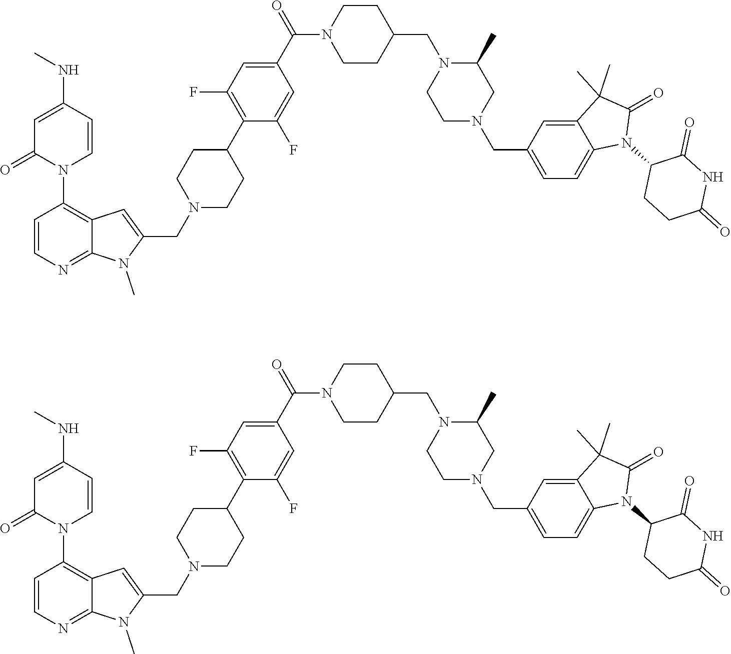 [SMILES: CNc1ccn(-c2ccnc3c2cc(CN2CCC(c4c(F)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)c(=O)c1 ; CNc1ccn(-c2ccnc3c2cc(CN2CCC(c4c(F)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)c(=O)c1]

### Example 3u205 and 3u206. (S)-3-(5-(((S)-4-((1-(3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)methyl)-3-methylpiperazin-1-yl)methyl)-3,3-dimethyl-2-oxoindolin-1-yl)piperidine-2,6-dione and (R)-3-(5-(((S)-4-((1-(3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)methyl)-3-methylpiperazin-1-yl)methyl)-3,3-dimethyl-2-oxoindolin-1-yl)piperidine-2,6-dione

Example 3u204 (85 mg) was resolved by HPLC using a Chiralpak IK 30×250 mm 5 μm column with an eluent of 35% ACN, 35% IPA and 30% DCM (35 mL/min) to give Example 3u205 (21 mg, first peak) and Example 3u206 (26 mg, second peak). The absolute configurations for these compounds at the glutarimide chiral centers were not determined.

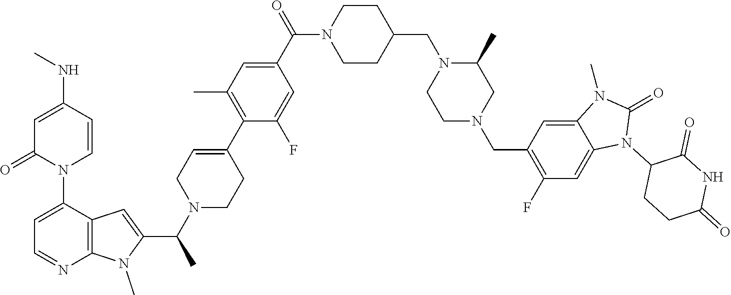 [SMILES: CNc1ccn(-c2ccnc3c2cc([C@H](C)N2CC=C(c4c(C)cc(C(=O)N5CCC(CN6CCN(Cc7cc8c(cc7F)n(C7CCC(=O)NC7=O)c(=O)n8C)C[C@@H]6C)CC5)cc4F)CC2)n3C)c(=O)c1]

Example 3u207. 3-(6-fluoro-5-(((S)-4-((1-(3-fluoro-5-methyl-4-(1-((S)-1-(1-methyl-4-(4-(methyl-amino)-2-oxopyridin-1 (2H)-yl)-1H-pyrrolo[2,3-b]pyridin-2-yl)ethyl)-1,2,3,6-tetrahydropyridin-4-yl)-benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione

Example 3u207 (0.20 g) was prepared similarly to Example 3u166 from Ints. 2G46/3E147 (0.32 g/0.48 g).

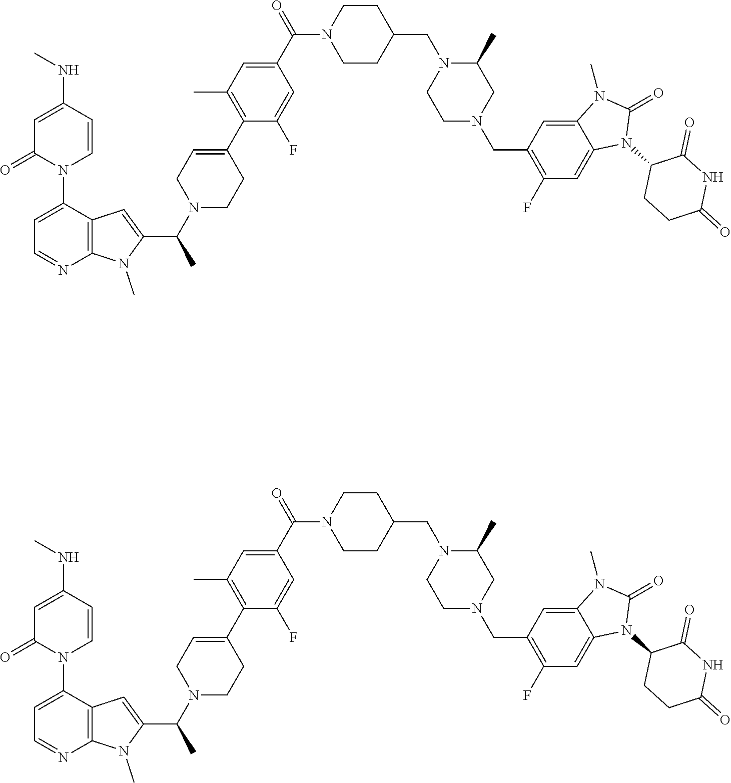 [SMILES: CNc1ccn(-c2ccnc3c2cc([C@H](C)N2CC=C(c4c(C)cc(C(=O)N5CCC(CN6CCN(Cc7cc8c(cc7F)n([C@H]7CCC(=O)NC7=O)c(=O)n8C)C[C@@H]6C)CC5)cc4F)CC2)n3C)c(=O)c1 ; CNc1ccn(-c2ccnc3c2cc([C@H](C)N2CC=C(c4c(C)cc(C(=O)N5CCC(CN6CCN(Cc7cc8c(cc7F)n([C@@H]7CCC(=O)NC7=O)c(=O)n8C)C[C@@H]6C)CC5)cc4F)CC2)n3C)c(=O)c1]

### Example 3u208 and 3u209. (S)-3-(6-fluoro-5-(((S)-4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione and (R)-3-(6-fluoro-5-(((S)-4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione

Example 3u207 (175 mg) was resolved by HPLC using a Chiralpak IK 30×250 mm 5 μm column with an eluent of 35% ACN, 35% IPA and 30% DCM (30 mL/min) to give Example 3u208 (30 mg, first peak) and Example 3u209 (30 mg, second peak). The absolute configurations for these compounds at the glutarimide chiral centers were not determined.

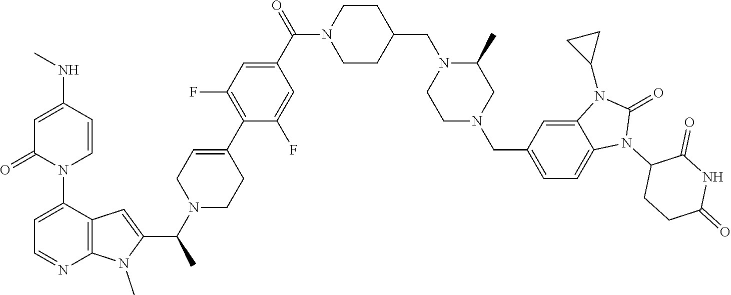 [SMILES: CNc1ccn(-c2ccnc3c2cc([C@H](C)N2CC=C(c4c(F)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)c(=O)c1]

### Example 3u210. 3-(3-cyclopropyl-5-(((S)-4-((1-(3,5-difluoro-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)-piperidin-4-yl)methyl)-3-methylpiperazin-1-yl)methyl)-2-oxo-2,3-dihydro-1H-benzo[d]imidazol-1-yl)piperidine-2,6-dione

Example 3u210 (0.15 g) was prepared similarly to Example 3u166 Ints. 2G118/3E78 (0.30 g/0.40 g).

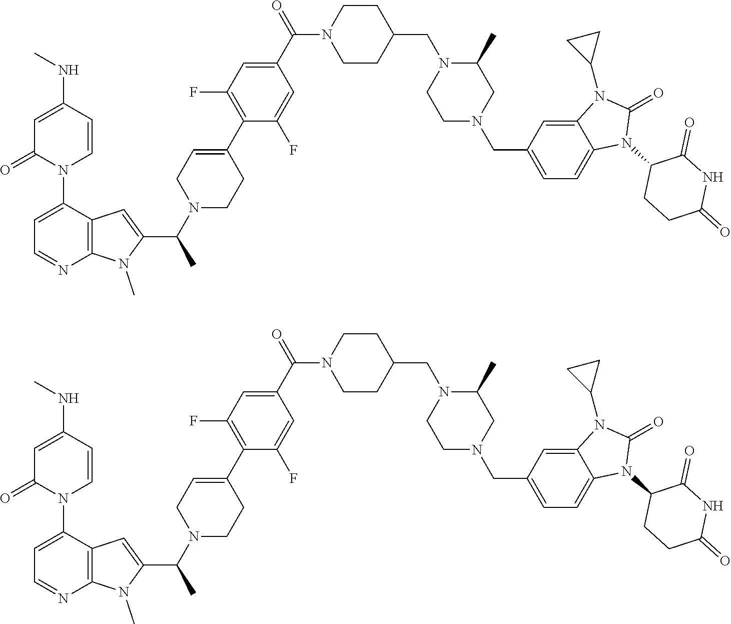 [SMILES: CNc1ccn(-c2ccnc3c2cc([C@H](C)N2CC=C(c4c(F)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)c(=O)c1 ; CNc1ccn(-c2ccnc3c2cc([C@H](C)N2CC=C(c4c(F)cc(C(=O)N5CCC(CN6CCN(Cc7ccc8c(c7)n(C7CC7)c(=O)n8[C@@H]7CCC(=O)NC7=O)C[C@@H]6C)CC5)cc4F)CC2)n3C)c(=O)c1]

### Example 3u211 and 3u212. (S)-3-(3-cyclopropyl-5-(((S)-4-((1-(3,5-difluoro-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione and (R)-3-(3-cyclopropyl-5-(((S)-4-((1-(3,5-difluoro-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione

Example 3u210 (100 mg) was resolved by HPLC using a Chiralpak IK 30×250 mm 5 μm column with an eluent of 35% ACN, 35% IPA and 30% DCM (28 mL/min) to give Example 3u211 (37 mg, first peak) and Example 3u212 (30 mg, second peak). The absolute configurations for these compounds at the glutarimide chiral centers were not determined.

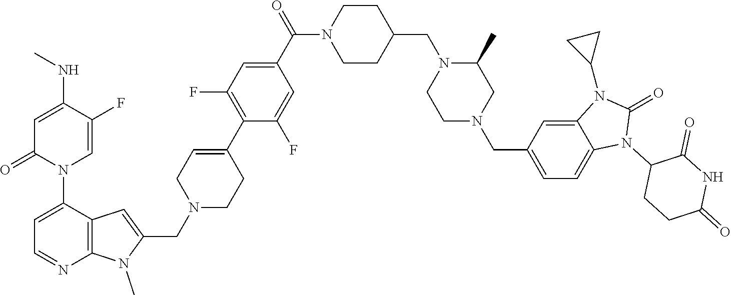 [SMILES: CNc1cc(=O)n(-c2ccnc3c2cc(CN2CC=C(c4c(F)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)cc1F]

### Example 3u213. 3-(3-cyclopropyl-5-(((S)-4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)-1,2,3,6-tetrahydropyridin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione

Example 3u213 (0.14 g) was prepared similarly to Example 3u166 from Ints. 2G48/3E78 (0.30 g/0.40 g).

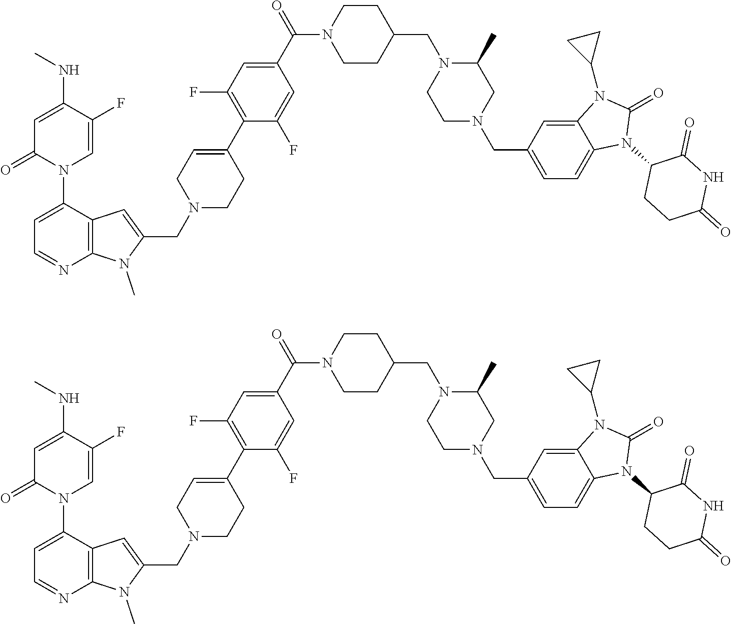 [SMILES: CNc1cc(=O)n(-c2ccnc3c2cc(CN2CC=C(c4c(F)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)cc1F ; CNc1cc(=O)n(-c2ccnc3c2cc(CN2CC=C(c4c(F)cc(C(=O)N5CCC(CN6CCN(Cc7ccc8c(c7)n(C7CC7)c(=O)n8[C@@H]7CCC(=O)NC7=O)C[C@@H]6C)CC5)cc4F)CC2)n3C)cc1F]

### Example 3u214 and 3u215. (S)-3-(3-cyclopropyl-5-(((S)-4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)-1,2,3,6-tetrahydropyridin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione and (R)-3-(3-cyclopropyl-5-(((S)-4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)-1,2,3,6-tetrahydropyridin-4-yl)benzoyl)piperidin-4-yl)methyl)-3-methylpiperazin-1-yl)methyl)-2-oxo-2,3-dihydro-1H-benzo[d]imidazol-1-yl)piperidine-2,6-dione

Example 3u213 (120 mg) was resolved by HPLC using a Chiralpak IK 30×250 mm 5 μm column with an eluent of 35% ACN, 35% IPA and 30% DCM (35 mL/min) to give Example 3u214 (26 mg, first peak) and Example 3u215 (28 mg, second peak). The absolute configurations for these compounds at the glutarimide chiral centers were not determined.

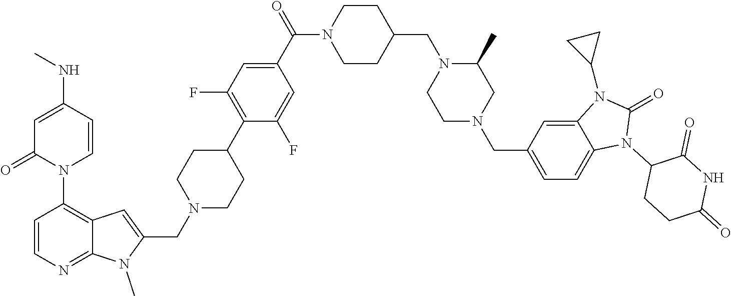 [SMILES: CNc1ccn(-c2ccnc3c2cc(CN2CCC(c4c(F)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)c(=O)c1]

### Example 3u216. 3-(3-cyclopropyl-5-(((S)-4-((1-(3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)-methyl)-3-methylpiperazin-1-yl)methyl)-2-oxo-2,3-dihydro-1H-benzo[d]imidazol-1-yl)piperidine-2,6-dione

Example 3u216 (0.14 g) was prepared similarly to Example 3u166 from Ints. 2G108/3E78 (0.30 g/0.41 g).

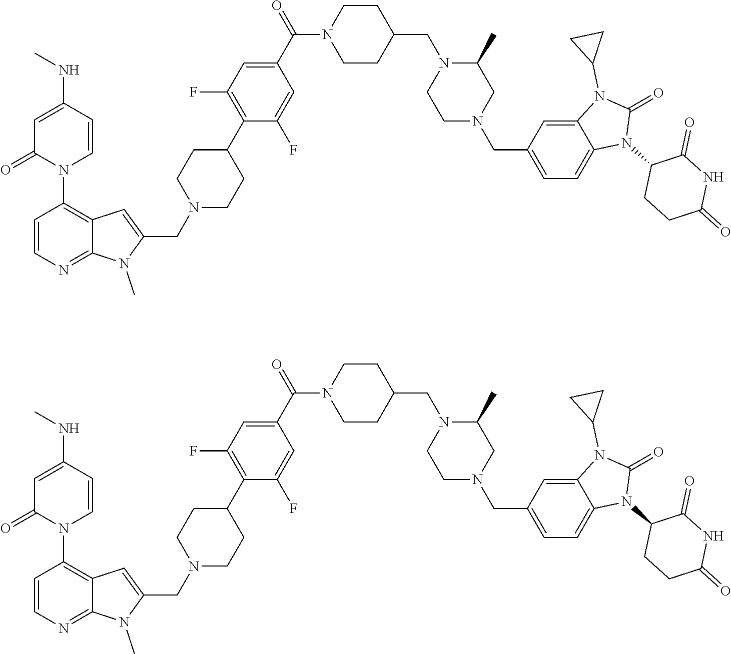 [SMILES: CNc1ccn(-c2ccnc3c2cc(CN2CCC(c4c(F)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)c(=O)c1 ; CNc1ccn(-c2ccnc3c2cc(CN2CCC(c4c(F)cc(C(=O)N5CCC(CN6CCN(Cc7ccc8c(c7)n(C7CC7)c(=O)n8[C@@H]7CCC(=O)NC7=O)C[C@@H]6C)CC5)cc4F)CC2)n3C)c(=O)c1]

### Example 3u217 and 3u218. (S)-3-(3-cyclopropyl-5-(((S)-4-((1-(3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)-piperidin-4-yl)methyl)-3-methylpiperazin-1-yl)methyl)-2-oxo-2,3-dihydro-1H-benzo[d]imidazol-1-yl)piperidine-2,6-dione and (R)-3-(3-cyclopropyl-5-(((S)-4-((1-(3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)-piperidin-4-yl)methyl)-3-methylpiperazin-1-yl)methyl)-2-oxo-2,3-dihydro-1H-benzo[d]imidazol-1-yl)piperidine-2,6-dione

Example 3u216 (100 mg) was resolved by HPLC using a Chiralpak IK 30×250 mm 5 μm column with an eluent of 35% ACN, 35% IPA and 30% DCM (50 mL/min) to give Example 3u217 (25 mg, first peak) and Example 3u218 (27 mg, second peak). The absolute configurations for these compounds at the glutarimide chiral centers were not determined.

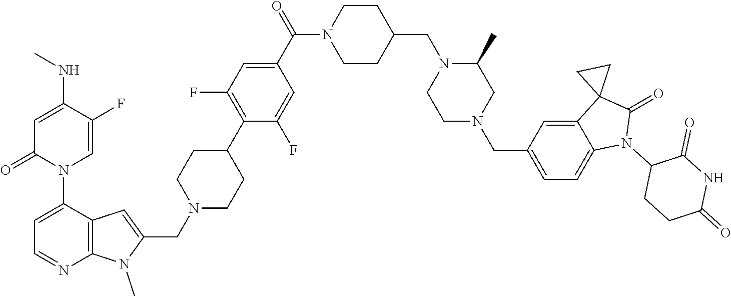 [SMILES: CNc1cc(=O)n(-c2ccnc3c2cc(CN2CCC(c4c(F)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)cc1F]

### Example 3u219. 3-(5′-(((S)-4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)methyl)-3-methylpiperazin-1-yl)methyl)-2′-oxospiro[cyclopropane-1,3′-indolin]-1′-yl)piperidine-2,6-dione

Example 3u219 (105 mg) was prepared similarly to Example 3u166 from Ints. 2G58/3C42 (0.30 g/0.44 g).

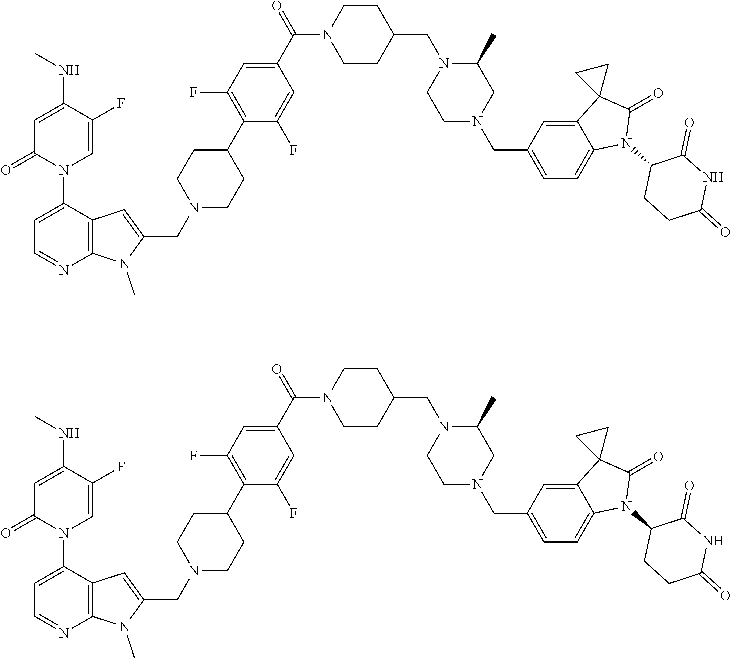 [SMILES: CNc1cc(=O)n(-c2ccnc3c2cc(CN2CCC(c4c(F)cc(C(=O)N5CCC(CN6CCN(Cc7ccc8c(c7)C7(CC7)C(=O)N8[C@H]7CCC(=O)NC7=O)C[C@@H]6C)CC5)cc4F)CC2)n3C)cc1F ; CNc1cc(=O)n(-c2ccnc3c2cc(CN2CCC(c4c(F)cc(C(=O)N5CCC(CN6CCN(Cc7ccc8c(c7)C7(CC7)C(=O)N8[C@@H]7CCC(=O)NC7=O)C[C@@H]6C)CC5)cc4F)CC2)n3C)cc1F]

### Example 3u220 and 3u221. (S)-3-(5′-(((S)-4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)methyl)-3-methylpiperazin-1-yl)methyl)-2′-oxospiro[cyclopropane-1,3′-indolin]-1′-yl)piperidine-2,6-dione and (R)-3-(5′-(((S)-4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)methyl)-3-methylpiperazin-1-yl)methyl)-2′-oxospiro[cyclopropane-1,3′-indolin]-1′-yl)piperidine-2,6-dione

Example 3u219 (85 mg) was resolved by HPLC using a Chiralpak IK 30×250 mm 5 μm column with an eluent of 35% ACN, 35% IPA and 30% DCM (28 mL/min) to give Example 3u220 (37 mg, first peak) and Example 3u221 (30 mg, second peak). The absolute configurations for these compounds at the glutarimide chiral centers were not determined.

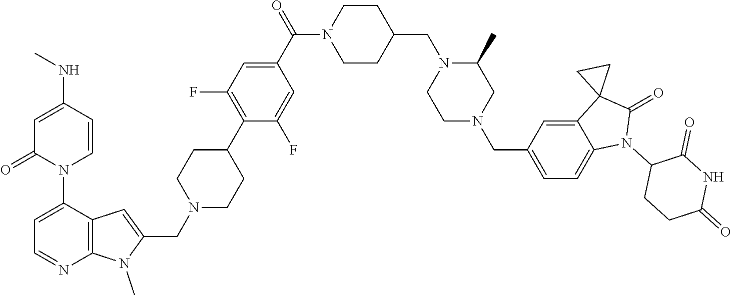 [SMILES: CNc1ccn(-c2ccnc3c2cc(CN2CCC(c4c(F)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)c(=O)c1]

### Example 3u222. 3-(5′-(((S)-4-((1-(3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)methyl)-3-methylpiperazin-1-yl)methyl)-2′-oxospiro[cyclopropane-1,3′-indolin]-1′-yl)piperidine-2,6-dione

Example 3u222 (0.12 g) was prepared similarly to Example 3u166 from Ints. 2G108/3C42 (0.30 g/0.46 g).

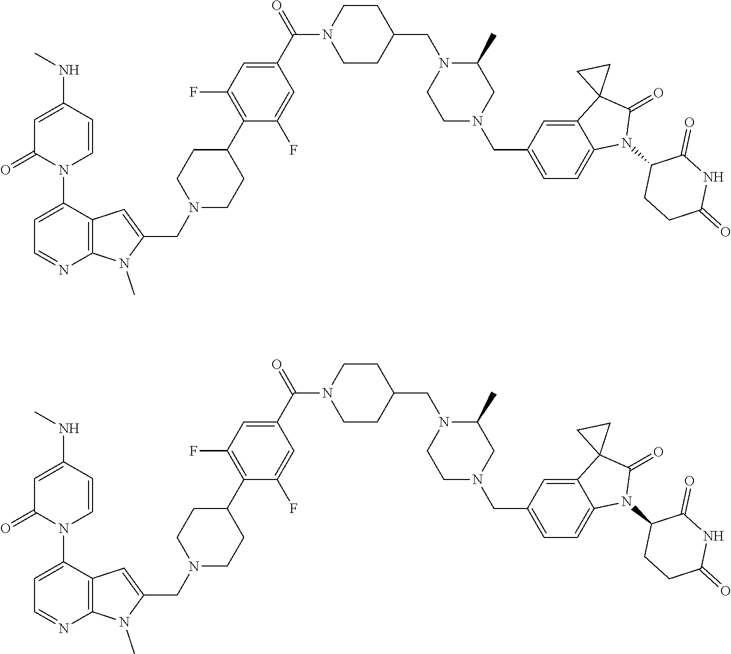 [SMILES: CNc1ccn(-c2ccnc3c2cc(CN2CCC(c4c(F)cc(C(=O)N5CCC(CN6CCN(Cc7ccc8c(c7)C7(CC7)C(=O)N8[C@H]7CCC(=O)NC7=O)C[C@@H]6C)CC5)cc4F)CC2)n3C)c(=O)c1 ; CNc1ccn(-c2ccnc3c2cc(CN2CCC(c4c(F)cc(C(=O)N5CCC(CN6CCN(Cc7ccc8c(c7)C7(CC7)C(=O)N8[C@@H]7CCC(=O)NC7=O)C[C@@H]6C)CC5)cc4F)CC2)n3C)c(=O)c1]

### Example 3u223 and 3u224. (S)-3-(5′-(((S)-4-((1-(3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)methyl)-3-methylpiperazin-1-yl)methyl)-2′-oxospiro[cyclopropane-1,3′-indolin]-1′-yl)piperidine-2,6-dione and (R)-3-(5′-(((S)-4-((1-(3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)methyl)-3-methylpiperazin-1-yl)methyl)-2′-oxospiro[cyclopropane-1,3′-indolin]-1′-yl)piperidine-2,6-dione

Example 3u222 (100 mg) was resolved by HPLC using a Chiralpak IK 30×250 mm 5 μm column with an eluent of 35% ACN, 35% IPA and 30% DCM (28 mL/min) to give Example 3u223 (40 mg, first peak) and Example 3u224 (35 mg, second peak). The absolute configurations for these compounds at the glutarimide chiral centers were not determined.

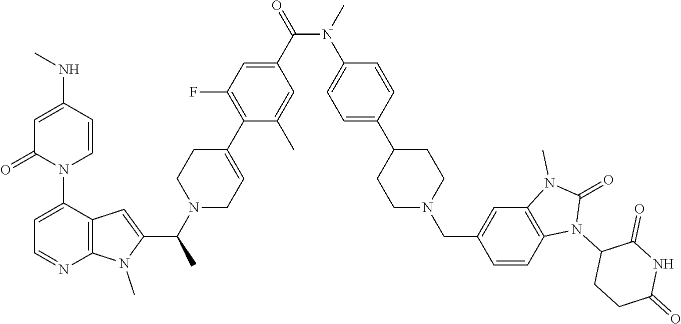 [SMILES: CNc1ccn(-c2ccnc3c2cc([C@H](C)N2CC=C(c4c(C)cc(C(=O)N(C)c5ccc(C6CCN(Cc7ccc8c(c7)n(C)c(=O)n8C7CCC(=O)NC7=O)CC6)cc5)cc4F)CC2)n3C)c(=O)c1]

### Example 3u225. N-(4-(1-((1-(2,6-dioxopiperidin-3-yl)-3-methyl-2-oxo-2,3-dihydro-1H-benzo[d]-imidazol-5-yl)methyl)piperidin-4-yl)phenyl)-3-fluoro-N,5-dimethyl-4-(1-((S)-1-(1-methyl-4-(4-(methyl-amino)-2-oxopyridin-1 (2H)-yl)-1H-pyrrolo[2,3-b]pyridin-2-yl)ethyl)-1,2,3,6-tetrahydropyridin-4-yl)-benzamide

Example 3u225 (22 mg) was prepared similarly to Example 3u166 from Ints. 2G46/3U69 (0.24 g/0.35 g).

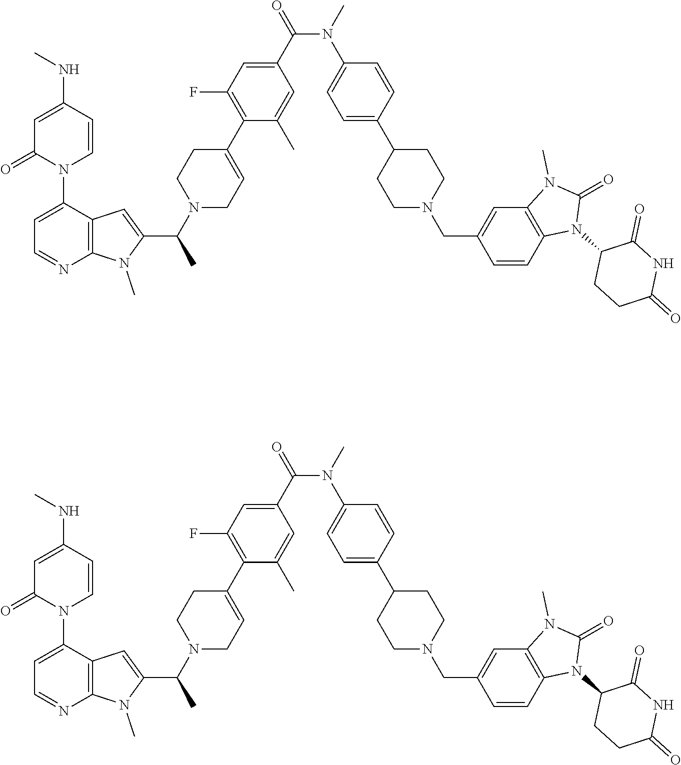 [SMILES: CNc1ccn(-c2ccnc3c2cc([C@H](C)N2CC=C(c4c(C)cc(C(=O)N(C)c5ccc(C6CCN(Cc7ccc8c(c7)n(C)c(=O)n8[C@H]7CCC(=O)NC7=O)CC6)cc5)cc4F)CC2)n3C)c(=O)c1 ; CNc1ccn(-c2ccnc3c2cc([C@H](C)N2CC=C(c4c(C)cc(C(=O)N(C)c5ccc(C6CCN(Cc7ccc8c(c7)n(C)c(=O)n8[C@@H]7CCC(=O)NC7=O)CC6)cc5)cc4F)CC2)n3C)c(=O)c1]

### Example 3u226 and 3u227. N-(4-(1-((1-((S)-2,6-dioxopiperidin-3-yl)-3-methyl-2-oxo-2,3-dihydro-1H-benzo[d]imidazol-5-yl)methyl)piperidin-4-yl)phenyl)-3-fluoro-N,5-dimethyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydro-pyridin-4-yl)benzamide and N-(4-(1-((1-((R)-2,6-dioxopiperidin-3-yl)-3-methyl-2-oxo-2,3-dihydro-1H-benzo[d]imidazol-5-yl)methyl)piperidin-4-yl)phenyl)-3-fluoro-N,5-dimethyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydro-pyridin-4-yl)benzamide

Example 3u225 (100 mg) was resolved by HPLC using a Chiralpak IK 250×30 mm 5 μm column with an eluent of 35% ACN, 35% IPA and 30% DCM (40 mL/min) to give Example 3u226 (7 mg, first peak) and Example 3u227 (7 mg, second peak). The absolute configurations for these compounds at the glutarimide chiral centers were not determined.

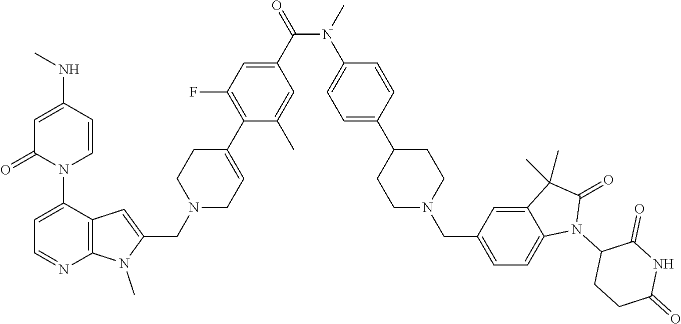 [SMILES: CNc1ccn(-c2ccnc3c2cc(CN2CC=C(c4c(C)cc(C(=O)N(C)c5ccc(C6CCN(Cc7ccc8c(c7)C(C)(C)C(=O)N8C7CCC(=O)NC7=O)CC6)cc5)cc4F)CC2)n3C)c(=O)c1]

### Example 3u228. N-(4-(1-((1-(2,6-dioxopiperidin-3-yl)-3,3-dimethyl-2-oxoindolin-5-yl)methyl)-piperidin-4-yl)phenyl)-3-fluoro-N,5-dimethyl-4-(1-((1-methyl-4-(4-(methylamino)-2-oxopyridin-1 (2H)-yl)-1H-pyrrolo[2,3-b]pyridin-2-yl)methyl)-1,2,3,6-tetrahydropyridin-4-yl)benzamide

Example 3u228 (60 mg) was prepared similarly to Example 3u166 from Ints. 1M60/3U102 (0.20 g/0.24 g).

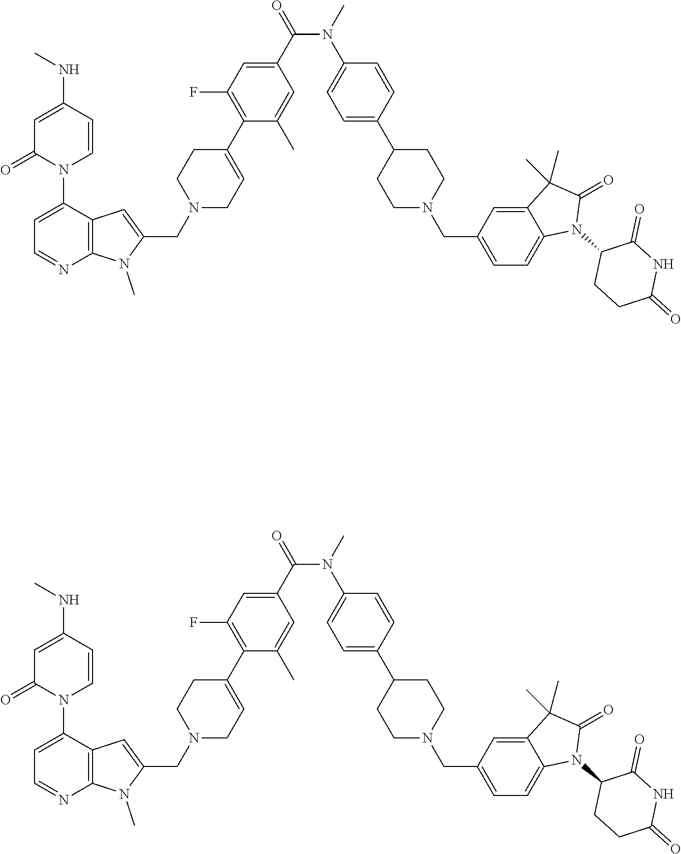 [SMILES: CNc1ccn(-c2ccnc3c2cc(CN2CC=C(c4c(C)cc(C(=O)N(C)c5ccc(C6CCN(Cc7ccc8c(c7)C(C)(C)C(=O)N8[C@H]7CCC(=O)NC7=O)CC6)cc5)cc4F)CC2)n3C)c(=O)c1 ; CNc1ccn(-c2ccnc3c2cc(CN2CC=C(c4c(C)cc(C(=O)N(C)c5ccc(C6CCN(Cc7ccc8c(c7)C(C)(C)C(=O)N8[C@@H]7CCC(=O)NC7=O)CC6)cc5)cc4F)CC2)n3C)c(=O)c1]

### Example 3u229 and 3u230. (S)—N-(4-(1-((1-(2,6-dioxopiperidin-3-yl)-3,3-dimethyl-2-oxoindolin-5-yl)methyl)piperidin-4-yl)phenyl)-3-fluoro-N,5-dimethyl-4-(1-((1-methyl-4-(4-(methylamino)-2-oxopyridin-1 (2H)-yl)-1H-pyrrolo[2,3-b]pyridin-2-yl)methyl)-1,2,3,6-tetrahydropyridin-4-yl)benzamide and (R)—N-(4-(1-((1-(2,6-dioxopiperidin-3-yl)-3,3-dimethyl-2-oxoindolin-5-yl)methyl)piperidin-4-yl)phenyl)-3-fluoro-N,5-dimethyl-4-(1-((1-methyl-4-(4-(methylamino)-2-oxopyridin-1 (2H)-yl)-1H-pyrrolo[2,3-b]pyridin-2-yl)methyl)-1,2,3,6-tetrahydropyridin-4-yl)benzamide

Example 3u228 (45 mg) was resolved by HPLC using a Chiralpak IK 250×30 mm 5 μm column with an eluent of 35% ACN, 35% IPA and 30% DCM (28 mL/min) to give Example 3u229 (17 mg, first peak) and Example 3u230 (18 mg, second peak). The absolute configurations for these compounds at the glutarimide chiral centers were not determined.

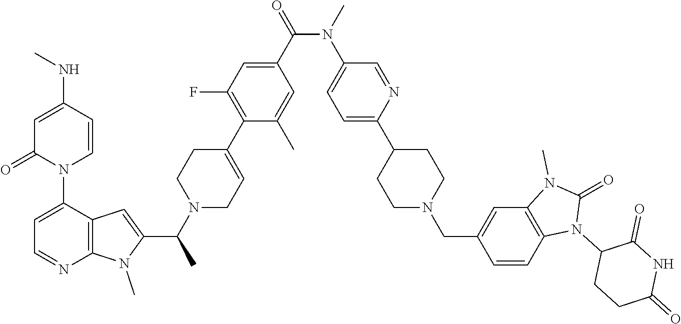 [SMILES: CNc1ccn(-c2ccnc3c2cc([C@H](C)N2CC=C(c4c(C)cc(C(=O)N(C)c5ccc(C6CCN(Cc7ccc8c(c7)n(C)c(=O)n8C7CCC(=O)NC7=O)CC6)nc5)cc4F)CC2)n3C)c(=O)c1]

### Example 3u231. N-(6-(1-((1-(2,6-dioxopiperidin-3-yl)-3-methyl-2-oxo-2,3-dihydro-1H-benzo[d]-imidazol-5-yl)methyl)piperidin-4-yl)pyridin-3-yl)-3-fluoro-N,5-dimethyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzamide

Example 3u231 (8 mg) was prepared similarly to Example 3u166 from Ints. 2G46/3U104 (0.10 g/0.12 g).

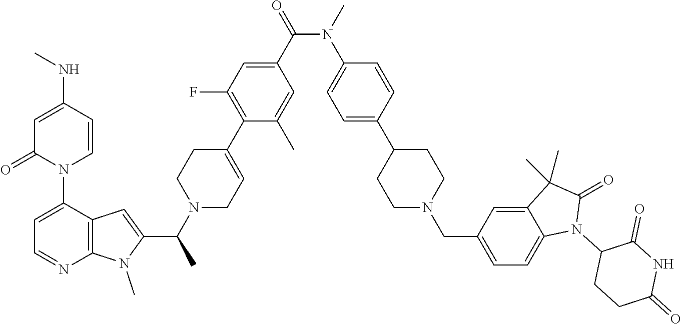 [SMILES: CNc1ccn(-c2ccnc3c2cc([C@H](C)N2CC=C(c4c(C)cc(C(=O)N(C)c5ccc(C6CCN(Cc7ccc8c(c7)C(C)(C)C(=O)N8C7CCC(=O)NC7=O)CC6)cc5)cc4F)CC2)n3C)c(=O)c1]

### Example 3u232. N-(4-(1-((1-(2,6-dioxopiperidin-3-yl)-3,3-dimethyl-2-oxoindolin-5-yl)methyl)-piperidin-4-yl)phenyl)-3-fluoro-N,5-dimethyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzamide

Example 3u232 (10 mg) was prepared similarly to Example 3u166 from Ints. 2G46/3U102 (50 mg/64 mg).

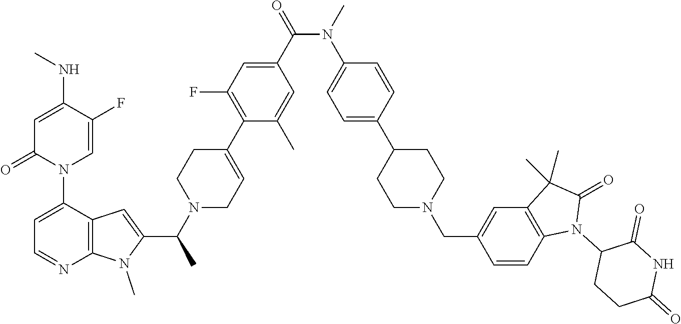 [SMILES: CNc1cc(=O)n(-c2ccnc3c2cc([C@H](C)N2CC=C(c4c(C)cc(C(=O)N(C)c5ccc(C6CCN(Cc7ccc8c(c7)C(C)(C)C(=O)N8C7CCC(=O)NC7=O)CC6)cc5)cc4F)CC2)n3C)cc1F]

### Example 3u233. N-(4-(1-((1-(2,6-dioxopiperidin-3-yl)-3,3-dimethyl-2-oxoindolin-5-yl)methyl)piperidin-4-yl)phenyl)-3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-N,5-dimethylbenzamide

Int. 2G45 (0.30 g) was dissolved in DMF/Et₃N (13:1, 4.8 mL). Int. 3U102 (0.34 g) and BEP (0.23 g) were added at 0° C. and the mixture stirred for 12 h at RT. The reaction mixture diluted with water to precipitate a solid that was HPLC-purified to give Example 3u233 (0.11 g).

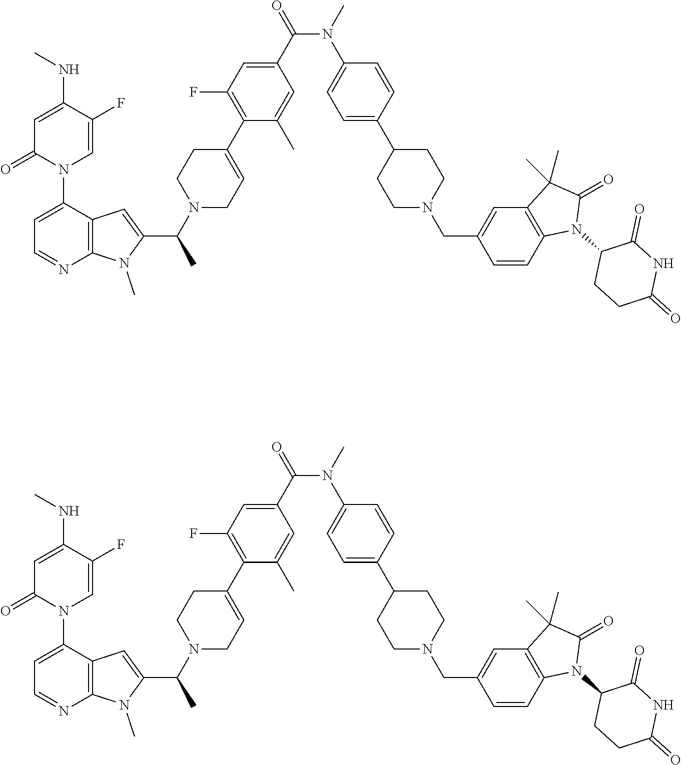 [SMILES: CNc1cc(=O)n(-c2ccnc3c2cc([C@H](C)N2CC=C(c4c(C)cc(C(=O)N(C)c5ccc(C6CCN(Cc7ccc8c(c7)C(C)(C)C(=O)N8[C@H]7CCC(=O)NC7=O)CC6)cc5)cc4F)CC2)n3C)cc1F ; CNc1cc(=O)n(-c2ccnc3c2cc([C@H](C)N2CC=C(c4c(C)cc(C(=O)N(C)c5ccc(C6CCN(Cc7ccc8c(c7)C(C)(C)C(=O)N8[C@@H]7CCC(=O)NC7=O)CC6)cc5)cc4F)CC2)n3C)cc1F]

### Example 3u234 and 3u235. N-(4-(1-((1-((S)-2,6-dioxopiperidin-3-yl)-3,3-dimethyl-2-oxoindolin-5-yl)methyl)piperidin-4-yl)phenyl)-3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-N,5-dimethylbenzamide and N-(4-(1-((1-((R)-2,6-dioxopiperidin-3-yl)-3,3-dimethyl-2-oxoindolin-5-yl)methyl)piperidin-4-yl)phenyl)-3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-N,5-dimethylbenzamide

Example 3u233 (72 mg) was resolved by HPLC using a Chiralpak IK 250×30 mm 5 μm column with an eluent of 35% ACN, 35% IPA and 30% DCM (26 mL/min) to give Example 3u234 (26 mg, first peak) and Example 3u235 (22 mg, second peak). The absolute configurations for these compounds at the glutarimide chiral centers were not determined.

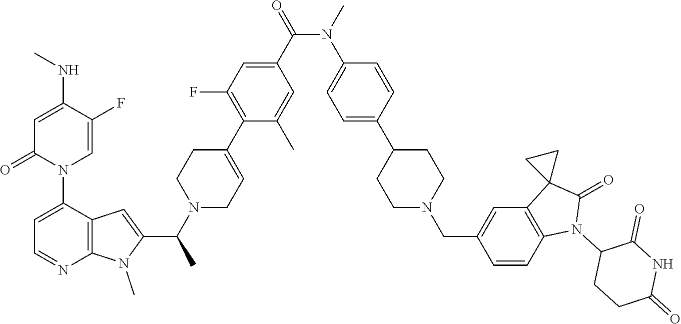 [SMILES: CNc1cc(=O)n(-c2ccnc3c2cc([C@H](C)N2CC=C(c4c(C)cc(C(=O)N(C)c5ccc(C6CCN(Cc7ccc8c(c7)C7(CC7)C(=O)N8C7CCC(=O)NC7=O)CC6)cc5)cc4F)CC2)n3C)cc1F]

### Example 3u236. N-(4-(1-((1′-(2,6-dioxopiperidin-3-yl)-2′-oxospiro[cyclopropane-1,3′-indolin]-5′-yl)methyl)piperidin-4-yl)phenyl)-3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-N,5-dimethylbenzamide

Ints. 2G45/3C55 (0.20 g/0.27 g) were mixed in DMF (5 mL). Et₃N (0.52 mL) and BEP (0.31 g) were added at 0° C. and the mixture stirred for 16 h at RT. The reaction mixture diluted with water to precipitate a solid that was HPLC-purified to give Example 3u236 (20 mg).

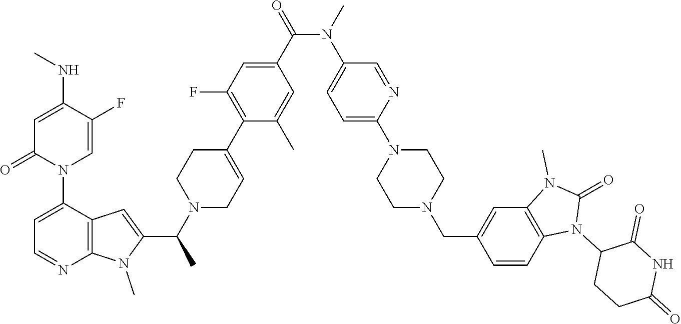 [SMILES: CNc1cc(=O)n(-c2ccnc3c2cc([C@H](C)N2CC=C(c4c(C)cc(C(=O)N(C)c5ccc(N6CCN(Cc7ccc8c(c7)n(C)c(=O)n8C7CCC(=O)NC7=O)CC6)nc5)cc4F)CC2)n3C)cc1F]

### Example 3u237. N-(6-(4-((1-(2,6-dioxopiperidin-3-yl)-3-methyl-2-oxo-2,3-dihydro-1H-benzo[d]-imidazol-5-yl)methyl)piperazin-1-yl)pyridin-3-yl)-3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-N,5-dimethylbenzamide

Example 3u237 (0.1 g) was prepared similarly to Example 3u236 from Ints. 2G45/3U96 (0.15 g/0.18 g).

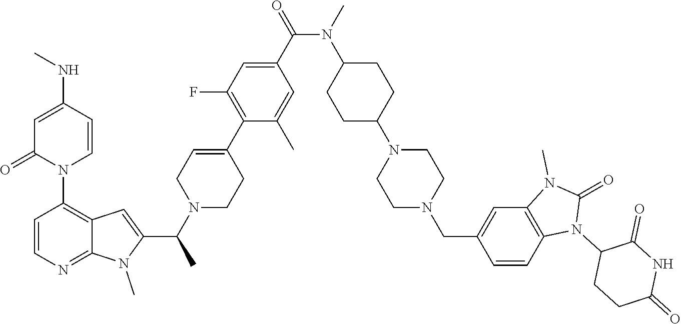 [SMILES: CNc1ccn(-c2ccnc3c2cc([C@H](C)N2CC=C(c4c(C)cc(C(=O)N(C)C5CCC(N6CCN(Cc7ccc8c(c7)n(C)c(=O)n8C7CCC(=O)NC7=O)CC6)CC5)cc4F)CC2)n3C)c(=O)c1]

### Example 3u238. N-(4-(4-((1-(2,6-dioxopiperidin-3-yl)-3-methyl-2-oxo-2,3-dihydro-1H-benzo[d]-imidazol-5-yl)methyl)piperazin-1-yl)cyclohexyl)-3-fluoro-N,5-dimethyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzamide

Int. 2G46 (21 mg) was added to a solution of Int. 3U97 (30 mg), HATU (21 mg) and DIPEA (52 μL) in DMF (1 mL) and stirred ON at RT. The reaction mixture was concentrated and HPLC-purified to give Example 3u238 (3 mg).

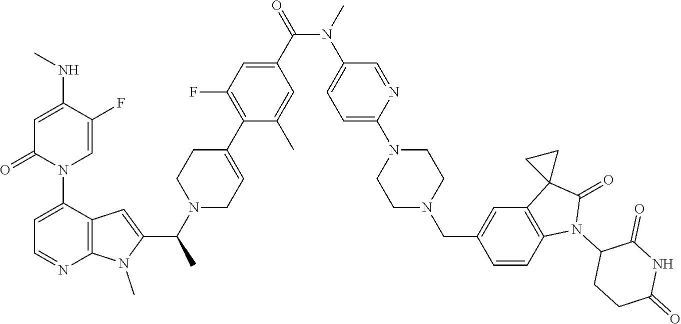 [SMILES: CNc1cc(=O)n(-c2ccnc3c2cc([C@H](C)N2CC=C(c4c(C)cc(C(=O)N(C)c5ccc(N6CCN(Cc7ccc8c(c7)C7(CC7)C(=O)N8C7CCC(=O)NC7=O)CC6)nc5)cc4F)CC2)n3C)cc1F]

### Example 3u239. N-(6-(4-((1′-(2,6-dioxopiperidin-3-yl)-2′-oxospiro[cyclopropane-1,3′-indolin]-5′-yl)-methyl)piperazin-1-yl)pyridin-3-yl)-3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-N,5-dimethyl-benzamide

Example 3u239 (75 mg) was prepared similarly to Example 3u236 from Ints. 2G45/3U114 (0.12 g/0.17 g).

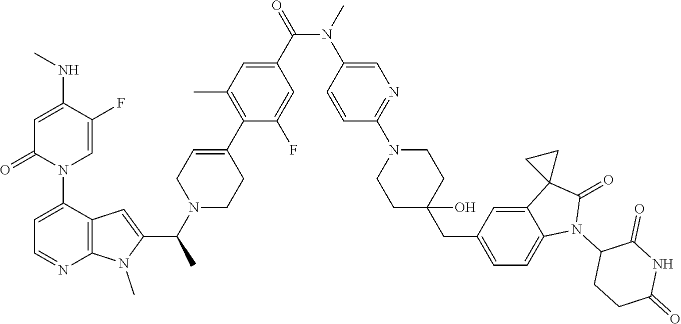 [SMILES: CNc1cc(=O)n(-c2ccnc3c2cc([C@H](C)N2CC=C(c4c(C)cc(C(=O)N(C)c5ccc(N6CCC(O)(Cc7ccc8c(c7)C7(CC7)C(=O)N8C7CCC(=O)NC7=O)CC6)nc5)cc4F)CC2)n3C)cc1F]

### Example 3u240. N-(6-(4-((1′-(2,6-dioxopiperidin-3-yl)-2′-oxospiro[cyclopropane-1,3′-indolin]-5′-yl)methyl)-4-hydroxypiperidin-1-yl)pyridin-3-yl)-3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-N,5-dimethylbenzamide

Example 3u240 (7 mg) was prepared similarly to Example 3u236 from Ints. 2G45/3U108 (50 mg/68 mg).

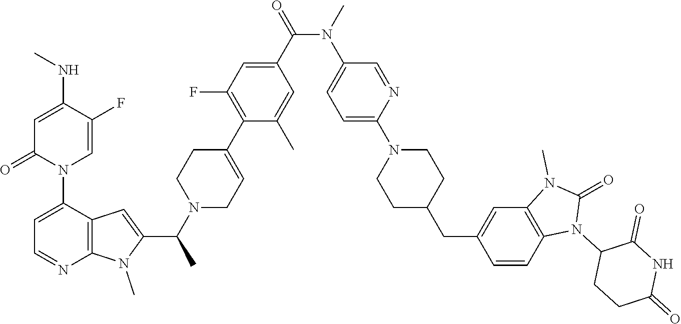 [SMILES: CNc1cc(=O)n(-c2ccnc3c2cc([C@H](C)N2CC=C(c4c(C)cc(C(=O)N(C)c5ccc(N6CCC(Cc7ccc8c(c7)n(C)c(=O)n8C7CCC(=O)NC7=O)CC6)nc5)cc4F)CC2)n3C)cc1F]

### Example 3u241. N-(6-(4-((1-(2,6-dioxopiperidin-3-yl)-3-methyl-2-oxo-2,3-dihydro-1H-benzo[d]-imidazol-5-yl)methyl)piperidin-1-yl)pyridin-3-yl)-3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-N,5-dimethylbenzamide

Example 3u241 (0.12 g) was prepared similarly to Example 3u236 from Ints. 2G45/3U104 (0.20 g/0.24 g).

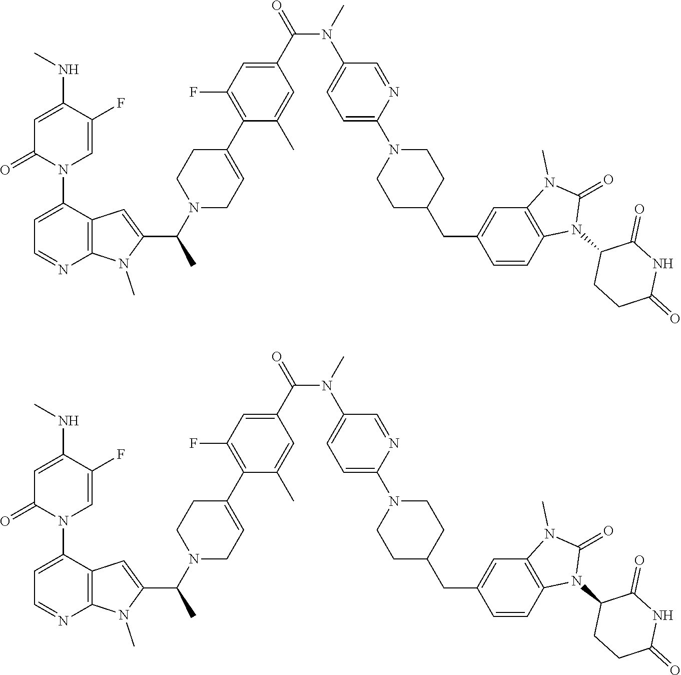 [SMILES: CNc1cc(=O)n(-c2ccnc3c2cc([C@H](C)N2CC=C(c4c(C)cc(C(=O)N(C)c5ccc(N6CCC(Cc7ccc8c(c7)n(C)c(=O)n8[C@H]7CCC(=O)NC7=O)CC6)nc5)cc4F)CC2)n3C)cc1F ; CNc1cc(=O)n(-c2ccnc3c2cc([C@H](C)N2CC=C(c4c(C)cc(C(=O)N(C)c5ccc(N6CCC(Cc7ccc8c(c7)n(C)c(=O)n8[C@@H]7CCC(=O)NC7=O)CC6)nc5)cc4F)CC2)n3C)cc1F]

### Example 3u242 and 3u243. N-(6-(4-((1-((S)-2,6-dioxopiperidin-3-yl)-3-methyl-2-oxo-2,3-dihydro-1H-benzo[d]imidazol-5-yl)methyl)piperidin-1-yl)pyridin-3-yl)-3-fluoro-4-(1-((S)-1-(4-(5-fluoro-4-(methyl-amino)-2-oxopyridin-1 (2H)-yl)-1-methyl-1H-pyrrolo[2,3-b]pyridin-2-yl)ethyl)-1,2,3,6-tetrahydro-pyridin-4-yl)-N,5-dimethylbenzamide and N-(6-(4-((1-((R)-2,6-dioxopiperidin-3-yl)-3-methyl-2-oxo-2,3-dihydro-1H-benzo[d]imidazol-5-yl)methyl)piperidin-1-yl)pyridin-3-yl)-3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-N,5-dimethylbenzamide

Example 3u241 (90 mg) was resolved by HPLC using a Chiralpak IK 250×10 mm 5 μm column with an eluent of 35% ACN, 35% IPA and 30% DCM (4 mL/min) to give Example 3u242 (42 mg, first peak) and Example 3u243 (18 mg, second peak). The absolute configurations were not determined at the glutarimide chiral centers for these compounds.

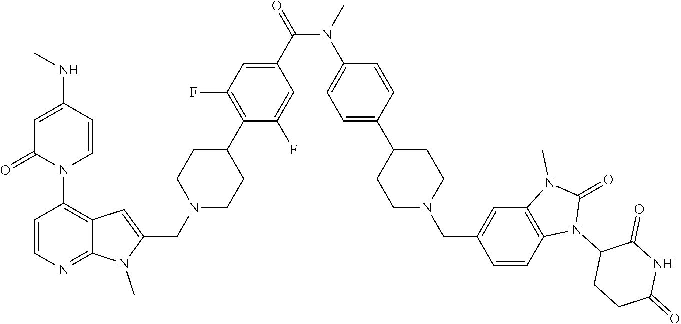 [SMILES: CNc1ccn(-c2ccnc3c2cc(CN2CCC(c4c(F)cc(C(=O)N(C)c5ccc(C6CCN(Cc7ccc8c(c7)n(C)c(=O)n8C7CCC(=O)NC7=O)CC6)cc5)cc4F)CC2)n3C)c(=O)c1]

### Example 3u244. N-(4-(1-((1-(2,6-dioxopiperidin-3-yl)-3-methyl-2-oxo-2,3-dihydro-1H-benzo[d]imidazol-5-yl)methyl)piperidin-4-yl)phenyl)-3,5-difluoro-N-methyl-4-(1-((1-methyl-4-(4-(methylamino)-2-oxopyridin-1 (2H)-yl)-1H-pyrrolo[2,3-b]pyridin-2-yl)methyl)piperidin-4-yl)benzamide

Example 3u244 (25 mg) was prepared similarly to Example 3u236 from Ints. 2G108/3U69 (0.10 g/0.13 g).

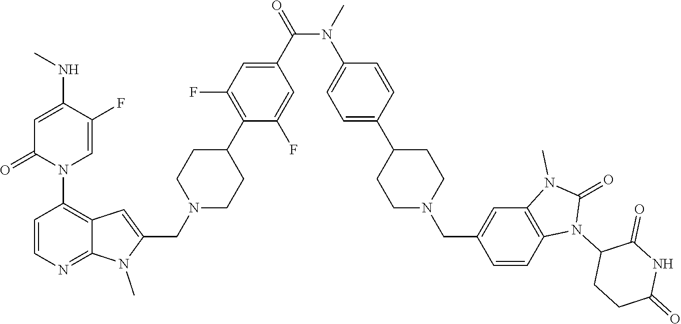 [SMILES: CNc1cc(=O)n(-c2ccnc3c2cc(CN2CCC(c4c(F)cc(C(=O)N(C)c5ccc(C6CCN(Cc7ccc8c(c7)n(C)c(=O)n8C7CCC(=O)NC7=O)CC6)cc5)cc4F)CC2)n3C)cc1F]

### Example 3u245. N-(4-(1-((1-(2,6-dioxopiperidin-3-yl)-3-methyl-2-oxo-2,3-dihydro-1H-benzo[d]imidazol-5-yl)methyl)piperidin-4-yl)phenyl)-3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)-N-methylbenzamide

Example 3u245 (22 mg) was prepared similarly to Example 3u236 from Ints. 2G58/3U69 (0.12 g/0.14 g).

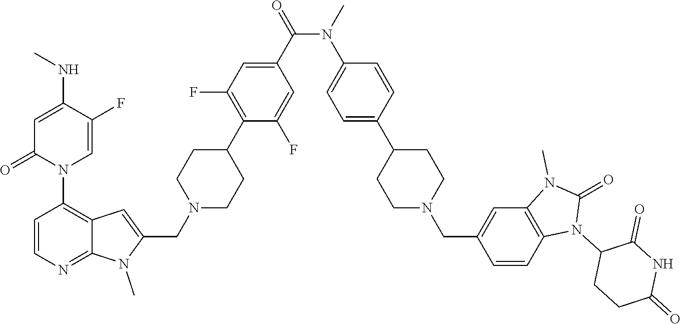 [SMILES: CNc1cc(=O)n(-c2ccnc3c2cc(CN2CCC(c4c(F)cc(C(=O)N(C)c5ccc(C6CCN(Cc7ccc8c(c7)n(C)c(=O)n8C7CCC(=O)NC7=O)CC6)cc5)cc4F)CC2)n3C)cc1F]

### Example 3u246. N-(6-(4-((1-(2,6-dioxopiperidin-3-yl)-3-methyl-2-oxo-2,3-dihydro-1H-benzo[d]imidazol-5-yl)methyl)-4-hydroxypiperidin-1-yl)pyridin-3-yl)-3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-N,5-dimethylbenzamide

Example 3u246 (98 mg) was prepared similarly to Example 3u236 from Ints. 2G45/3U116 (0.20 g/0.25 g).

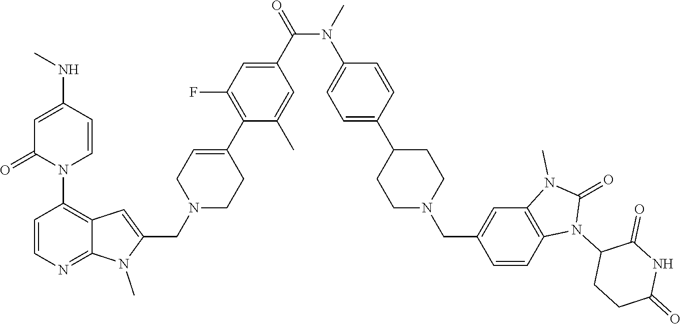 [SMILES: CNc1ccn(-c2ccnc3c2cc(CN2CC=C(c4c(C)cc(C(=O)N(C)c5ccc(C6CCN(Cc7ccc8c(c7)n(C)c(=O)n8C7CCC(=O)NC7=O)CC6)cc5)cc4F)CC2)n3C)c(=O)c1]

### Example 3u247. N-(4-(1-((1-(2,6-dioxopiperidin-3-yl)-3-methyl-2-oxo-2,3-dihydro-1H-benzo[d]-imidazol-5-yl)methyl)piperidin-4-yl)phenyl)-3-fluoro-N,5-dimethyl-4-(1-((1-methyl-4-(4-(methyl-amino)-2-oxopyridin-1 (2H)-yl)-1H-pyrrolo[2,3-b]pyridin-2-yl)methyl)-1,2,3,6-tetrahydropyridin-4-yl)benzamide

Example 3u247 (56 mg) was prepared similarly to Example 3u246 from Ints. 1M60/3U69 (0.10 g/0.13 g) in DMF (2 mL).

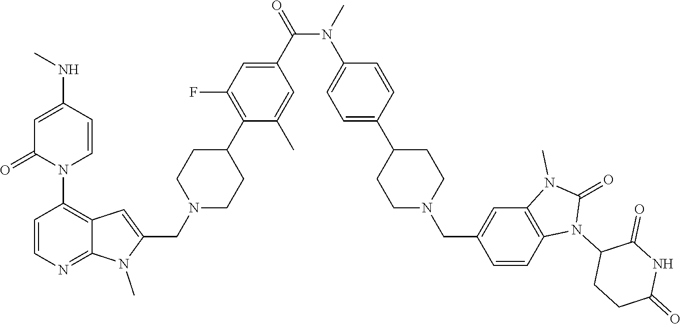 [SMILES: CNc1ccn(-c2ccnc3c2cc(CN2CCC(c4c(C)cc(C(=O)N(C)c5ccc(C6CCN(Cc7ccc8c(c7)n(C)c(=O)n8C7CCC(=O)NC7=O)CC6)cc5)cc4F)CC2)n3C)c(=O)c1]

### Example 3u248. N-(4-(1-((1-(2,6-dioxopiperidin-3-yl)-3-methyl-2-oxo-2,3-dihydro-1H-benzo[d]imidazol-5-yl)methyl)piperidin-4-yl)phenyl)-3-fluoro-N,5-dimethyl-4-(1-((1-methyl-4-(4-(methylamino)-2-oxopyridin-1 (2H)-yl)-1H-pyrrolo[2,3-b]pyridin-2-yl)methyl)piperidin-4-yl)benzamide

Example 3u248 (20 mg) was prepared similarly to Example 3u236 from Ints. 2G113/3U69 (0.13 g/0.15 g).

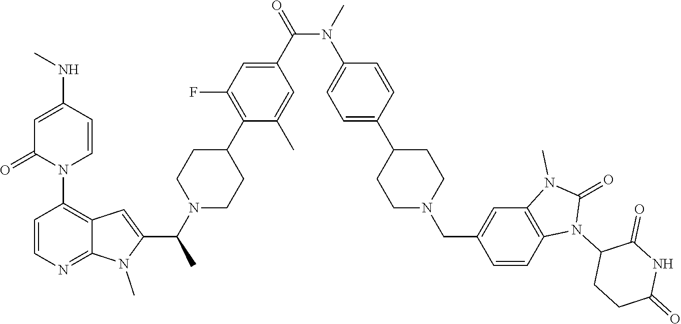 [SMILES: CNc1ccn(-c2ccnc3c2cc([C@H](C)N2CCC(c4c(C)cc(C(=O)N(C)c5ccc(C6CCN(Cc7ccc8c(c7)n(C)c(=O)n8C7CCC(=O)NC7=O)CC6)cc5)cc4F)CC2)n3C)c(=O)c1]

### Example 3u249. N-(4-(1-((1-(2,6-dioxopiperidin-3-yl)-3-methyl-2-oxo-2,3-dihydro-1H-benzo[d]-imidazol-5-yl)methyl)piperidin-4-yl)phenyl)-3-fluoro-N,5-dimethyl-4-(1-((S)-1-(1-methyl-4-(4-(methyl-amino)-2-oxopyridin-1 (2H)-yl)-1H-pyrrolo[2,3-b]pyridin-2-yl)ethyl)piperidin-4-yl)benzamide

Example 3u249 (8 mg) was prepared similarly to Example 3u236 from Ints. 2G54/3U69 (0.10 g/0.13 g).

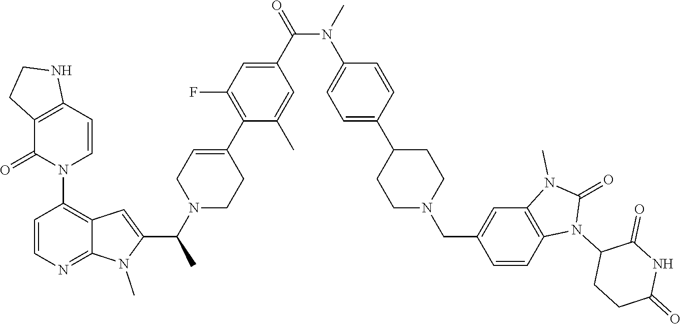 [SMILES: Cc1cc(C(=O)N(C)c2ccc(C3CCN(Cc4ccc5c(c4)n(C)c(=O)n5C4CCC(=O)NC4=O)CC3)cc2)cc(F)c1C1=CCN([C@@H](C)c2cc3c(-n4ccc5c(c4=O)CCN5)ccnc3n2C)CC1]

### Example 3u250. N-(4-(1-((1-(2,6-dioxopiperidin-3-yl)-3-methyl-2-oxo-2,3-dihydro-1H-benzo[d]imidazol-5-yl)methyl)piperidin-4-yl)phenyl)-3-fluoro-N,5-dimethyl-4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)-1,2,3,6-tetrahydropyridin-4-yl)benzamide

Example 3u250 (25 mg) was prepared similarly to Example 3u236 from Ints. 2G53/3U69 (0.30 g/0.31 g).

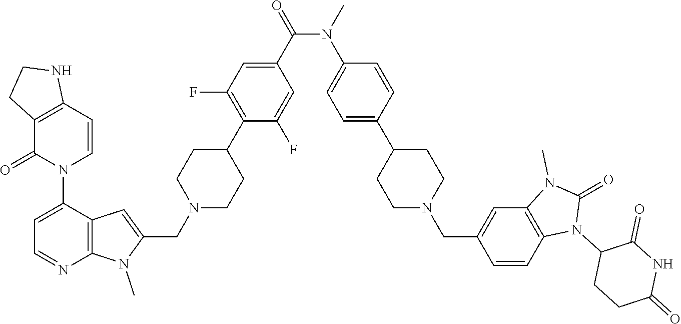 [SMILES: CN(C(=O)c1cc(F)c(C2CCN(Cc3cc4c(-n5ccc6c(c5=O)CCN6)ccnc4n3C)CC2)c(F)c1)c1ccc(C2CCN(Cc3ccc4c(c3)n(C)c(=O)n4C3CCC(=O)NC3=O)CC2)cc1]

### Example 3u251. N-(4-(1-((1-(2,6-dioxopiperidin-3-yl)-3-methyl-2-oxo-2,3-dihydro-1H-benzo[d]-imidazol-5-yl)methyl)piperidin-4-yl)phenyl)-3,5-difluoro-N-methyl-4-(1-((1-methyl-4-(4-oxo-1,2,3,4-tetrahydro-5H-pyrrolo[3,2-c]pyridin-5-yl)-1H-pyrrolo[2,3-b]pyridin-2-yl)methyl)piperidin-4-yl)-benzamide

Example 3u251 (25 mg) was prepared similarly to Example 3u236 from Ints. 2G57/3U69 (0.20 g/0.19 g).

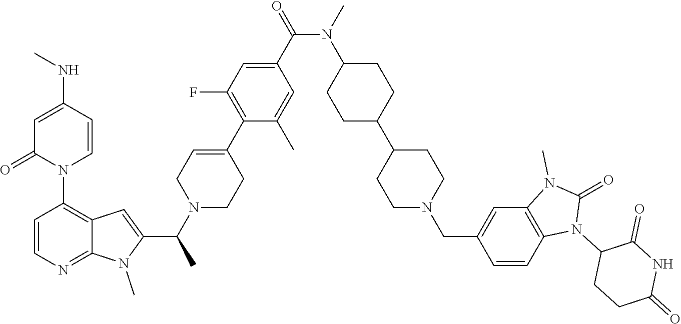 [SMILES: CNc1ccn(-c2ccnc3c2cc([C@H](C)N2CC=C(c4c(C)cc(C(=O)N(C)C5CCC(C6CCN(Cc7ccc8c(c7)n(C)c(=O)n8C7CCC(=O)NC7=O)CC6)CC5)cc4F)CC2)n3C)c(=O)c1]

### Example 3u252. N-(4-(1-((1-(2,6-dioxopiperidin-3-yl)-3-methyl-2-oxo-2,3-dihydro-1H-benzo[d]-imidazol-5-yl)methyl)piperidin-4-yl)cyclohexyl)-3-fluoro-N,5-dimethyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzamide

Ints. 3U67/3U135 (64 mg/0.15 g) and 4 Å MS were stirred 24 h in DCE/Et₃N (3 mL/50 microL). NaCNBH3 (19 mg) was added and stirring continued 24 h. The mixture was filtered. The residue after concentration was HPLC-purified to give Example 3u252 (5.1 mg).

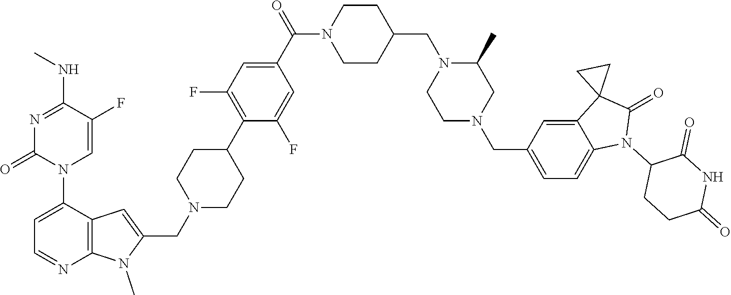 [SMILES: CNc1nc(=O)n(-c2ccnc3c2cc(CN2CCC(c4c(F)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)cc1F]

### Example 3h10. 3-(5′-(((S)-4-((1-(3,5-difluoro-4-(1-((4-(5-fluoro-4-(methylamino)-2-oxopyrimidin-1 (2H)-yl)-1-methyl-1H-pyrrolo[2,3-b]pyridin-2-yl)methyl)piperidin-4-yl)benzoyl)piperidin-4-yl)methyl)-3-methylpiperazin-1-yl)methyl)-2′-oxospiro[cyclopropane-1,3′-indolin]-1′-yl)piperidine-2,6-dione

Example 3h10 (26 mg) was prepared similarly to Example 3h2 from Ints. 3C40/3C42 (0.10 g/0.12 g).

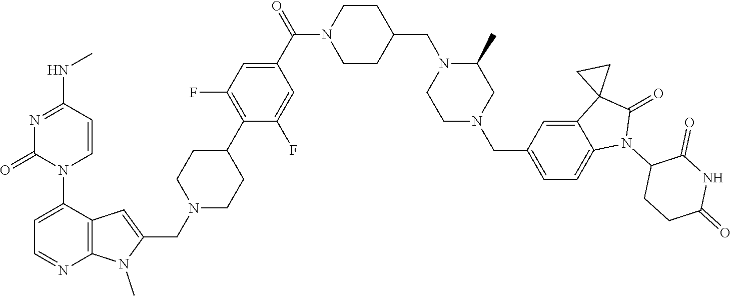 [SMILES: CNc1ccn(-c2ccnc3c2cc(CN2CCC(c4c(F)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)c(=O)n1]

### Example 3h12. 3-(5′-(((S)-4-((1-(3,5-difluoro-4-(1-((1-methyl-4-(4-(methylamino)-2-oxopyrimidin-1 (2H)-yl)-1H-pyrrolo[2,3-b]pyridin-2-yl)methyl)piperidin-4-yl)benzoyl)piperidin-4-yl)methyl)-3-methylpiperazin-1-yl)methyl)-2′-oxospiro[cyclopropane-1,3′-indolin]-1′-yl)piperidine-2,6-dione

Example 3h12 (27 mg) was prepared similarly to Example 3h2 from Ints. 3C10/3C42 (0.10 g/0.13 g).

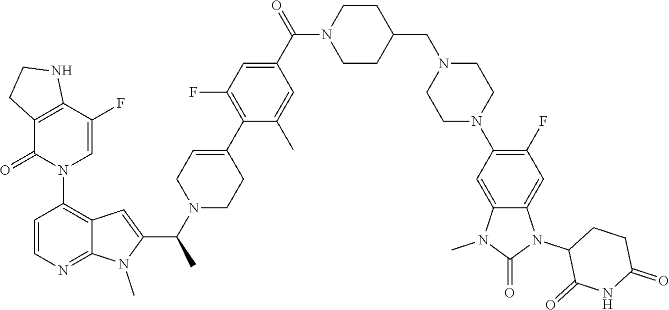 [SMILES: Cc1cc(C(=O)N2CCC(CN3CCN(c4cc5c(cc4F)n(C4CCC(=O)NC4=O)c(=O)n5C)CC3)CC2)cc(F)c1C1=CCN([C@@H](C)c2cc3c(-n4cc(F)c5c(c4=O)CCN5)ccnc3n2C)CC1]

### Example 3u253. 3-(6-fluoro-5-(4-((1-(3-fluoro-4-(1-((S)-1-(4-(7-fluoro-4-oxo-1,2,3,4-tetrahydro-5H-pyrrolo[3,2-c]pyridin-5-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)-3-methyl-2-oxo-2,3-dihydro-1H-benzo[d]imidazol-1-yl)piperidine-2,6-dione

Example 3u253 (20 mg) was prepared similarly to Example 3u163 from Ints. 2G27/2T37 (0.12 g/0.15 g).

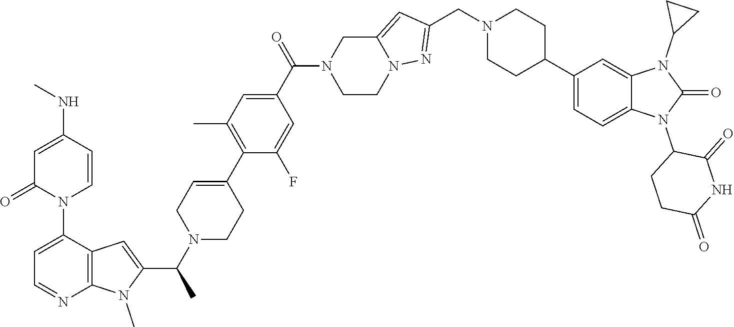 [SMILES: CNc1ccn(-c2ccnc3c2cc([C@H](C)N2CC=C(c4c(C)cc(C(=O)N5CCn6nc(CN7CCC(c8ccc9c(c8)n(C8CC8)c(=O)n9C8CCC(=O)NC8=O)CC7)cc6C5)cc4F)CC2)n3C)c(=O)c1]

### Example 3u254. 3-(3-cyclopropyl-5-(1-((5-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)-4,5,6,7-tetrahydropyrazolo[1,5-a]pyrazin-2-yl)methyl)piperidin-4-yl)-2-oxo-2,3-dihydro-1H-benzo[d]imidazol-1-yl)piperidine-2,6-dione

Example 3u254 (10 mg) was prepared similarly to Example 3u35 from Ints. 2G46/2T52 (60 mg/60 mg).

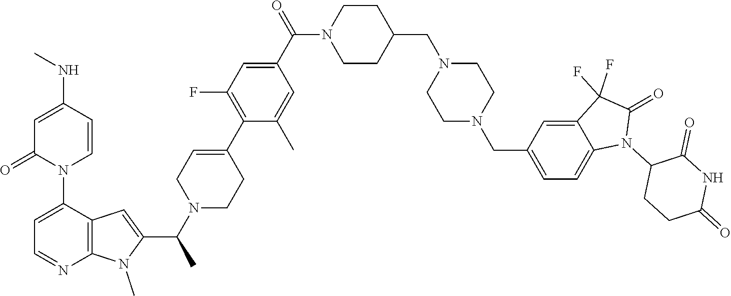 [SMILES: CNc1ccn(-c2ccnc3c2cc([C@H](C)N2CC=C(c4c(C)cc(C(=O)N5CCC(CN6CCN(Cc7ccc8c(c7)C(F)(F)C(=O)N8C7CCC(=O)NC7=O)CC6)CC5)cc4F)CC2)n3C)c(=O)c1]

### Example 3u255. 3-(3,3-difluoro-5-((4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)methyl)-2-oxoindolin-1-yl)piperidine-2,6-dione

Example 3u255 (4 mg) was prepared similarly to Example 3u35 from Ints. 2G46/2T70 (0.17 g/0.28 g).

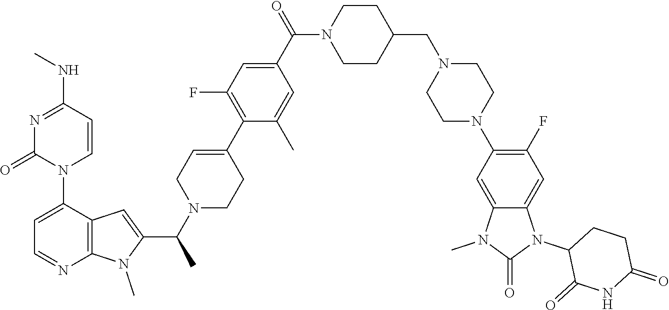 [SMILES: CNc1ccn(-c2ccnc3c2cc([C@H](C)N2CC=C(c4c(C)cc(C(=O)N5CCC(CN6CCN(c7cc8c(cc7F)n(C7CCC(=O)NC7=O)c(=O)n8C)CC6)CC5)cc4F)CC2)n3C)c(=O)n1]

### Example 3u256. 3-(6-fluoro-5-(4-((1-(3-fluoro-5-methyl-4-(1-((S)-1-(1-methyl-4-(4-(methylamino)-2-oxopyrimidin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)-3-methyl-2-oxo-2,3-dihydro-1H-benzo[d]imidazol-1-yl)piperidine-2,6-dione

Example 3u256 (10 mg) was prepared similarly to Example 3u163 from Ints. 2G108/2T37 (90 mg/0.10 g).

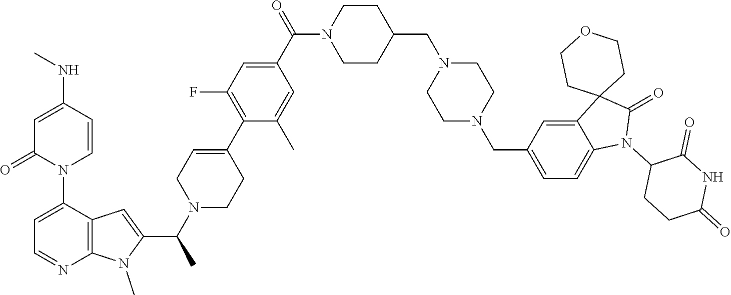 [SMILES: CNc1ccn(-c2ccnc3c2cc([C@H](C)N2CC=C(c4c(C)cc(C(=O)N5CCC(CN6CCN(Cc7ccc8c(c7)C7(CCOCC7)C(=O)N8C7CCC(=O)NC7=O)CC6)CC5)cc4F)CC2)n3C)c(=O)c1]

### Example 3u257. 3-(5-((4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)-piperidin-4-yl)methyl)piperazin-1-yl)methyl)-2-oxo-2′,3′,5′,6′-tetrahydrospiro[indoline-3,4′-pyran]-1-yl)piperidine-2,6-dione

Example 3u257 (4 mg) was prepared similarly to Example 3u35 from Ints. 2G46/2T74 (30 mg/30 mg).

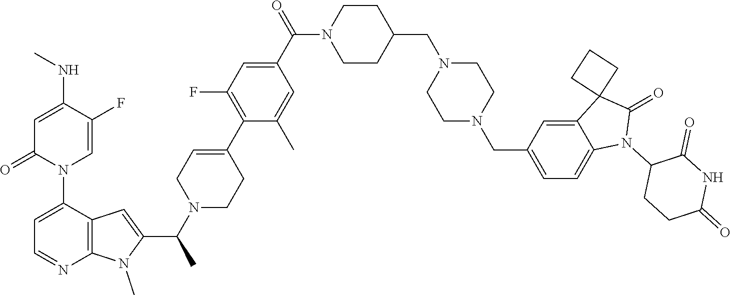 [SMILES: CNc1cc(=O)n(-c2ccnc3c2cc([C@H](C)N2CC=C(c4c(C)cc(C(=O)N5CCC(CN6CCN(Cc7ccc8c(c7)C7(CCC7)C(=O)N8C7CCC(=O)NC7=O)CC6)CC5)cc4F)CC2)n3C)cc1F]

### Example 3u258. 3-(5′-((4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)methyl)-2′-oxospiro[cyclobutane-1,3′-indolin]-1′-yl)piperidine-2,6-dione

Example 3u258 (42 mg) was prepared similarly to Example 3u163 from Ints. 2G45/2T39 (90 mg/0.10 g).

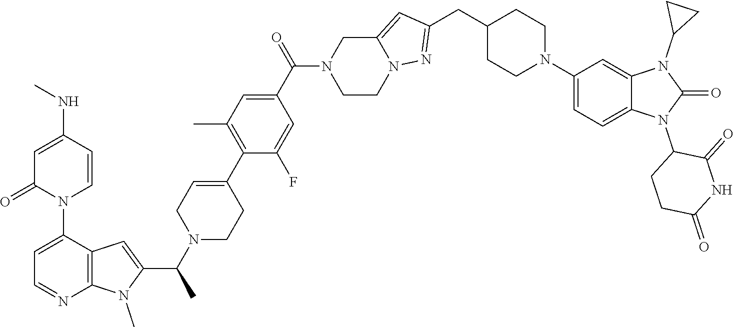 [SMILES: CNc1ccn(-c2ccnc3c2cc([C@H](C)N2CC=C(c4c(C)cc(C(=O)N5CCn6nc(CC7CCN(c8ccc9c(c8)n(C8CC8)c(=O)n9C8CCC(=O)NC8=O)CC7)cc6C5)cc4F)CC2)n3C)c(=O)c1]

### Example 3u259. 3-(3-cyclopropyl-5-(4-((5-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)-4,5,6,7-tetrahydropyrazolo[1,5-a]pyrazin-2-yl)methyl)piperidin-1-yl)-2-oxo-2,3-dihydro-1H-benzo[d]imidazol-1-yl)piperidine-2,6-dione

Example 3u259 (6 mg) was prepared similarly to Example 3u35 from Ints. 2G46/2T66 (26 mg/25 mg).

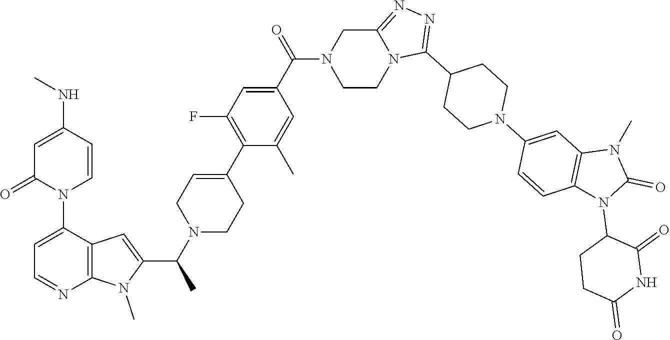 [SMILES: CNc1ccn(-c2ccnc3c2cc([C@H](C)N2CC=C(c4c(C)cc(C(=O)N5CCn6c(nnc6C6CCN(c7ccc8c(c7)n(C)c(=O)n8C7CCC(=O)NC7=O)CC6)C5)cc4F)CC2)n3C)c(=O)c1]

### Example 3u260. 3-(5-(4-(7-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)-5,6,7,8-tetrahydro-[1,2,4]triazolo[4,3-a]pyrazin-3-yl)piperidin-1-yl)-3-methyl-2-oxo-2,3-dihydro-1H-benzo[d]imidazol-1-yl)piperidine-2,6-dione

Example 3u260 (3 mg) was prepared similarly to Example 3u35 from Ints. 2G46/2T82 (28 mg/25 mg).

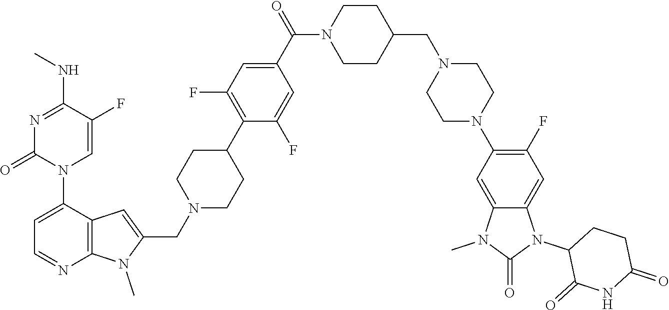 [SMILES: CNc1nc(=O)n(-c2ccnc3c2cc(CN2CCC(c4c(F)cc(C(=O)N5CCC(CN6CCN(c7cc8c(cc7F)n(C7CCC(=O)NC7=O)c(=O)n8C)CC6)CC5)cc4F)CC2)n3C)cc1F]

### Example 3u261. 3-(5-(4-((1-(3,5-difluoro-4-(1-((4-(5-fluoro-4-(methylamino)-2-oxopyrimidin-1 (2H)-yl)-1-methyl-1H-pyrrolo[2,3-b]pyridin-2-yl)methyl)piperidin-4-yl)benzoyl)piperidin-4-yl)methyl)piperazin-1-yl)-6-fluoro-3-methyl-2-oxo-2,3-dihydro-1H-benzo[d]imidazol-1-yl)piperidine-2,6-dione

Example 3u261 (30 mg) was prepared similarly to Example 3u163 from Ints. 3C40/2T37 (0.10 g/0.12 g).

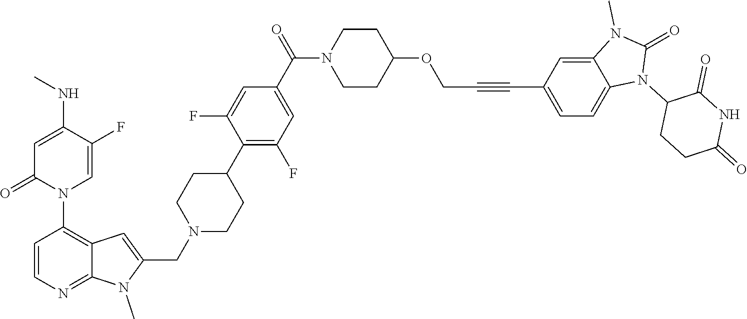 [SMILES: CNc1cc(=O)n(-c2ccnc3c2cc(CN2CCC(c4c(F)cc(C(=O)N5CCC(OCC#Cc6ccc7c(c6)n(C)c(=O)n7C6CCC(=O)NC6=O)CC5)cc4F)CC2)n3C)cc1F]

### Example 3u262. 3-(5-(3-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)oxy)prop-1-yn-1-yl)-3-methyl-2-oxo-2,3-dihydro-1H-benzo[d]imidazol-1-yl)piperidine-2,6-dione

Example 3u262 (30 mg) was prepared similarly to Example 3u163 from Ints. 2G58/3B1 (0.13 g/0.25 g).

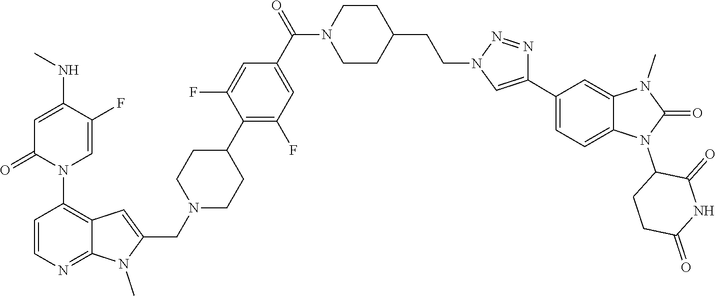 [SMILES: CNc1cc(=O)n(-c2ccnc3c2cc(CN2CCC(c4c(F)cc(C(=O)N5CCC(CCn6cc(-c7ccc8c(c7)n(C)c(=O)n8C7CCC(=O)NC7=O)nn6)CC5)cc4F)CC2)n3C)cc1F]

### Example 3u263. 3-(5-(1-(2-(1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)ethyl)-1H-1,2,3-triazol-4-yl)-3-methyl-2-oxo-2,3-dihydro-1H-benzo[d]imidazol-1-yl)piperidine-2,6-dione

Example 3u263 (52 mg) was prepared similarly to Example 3u163 from Ints. 2G58/2T63 (0.12 g/0.16 g).

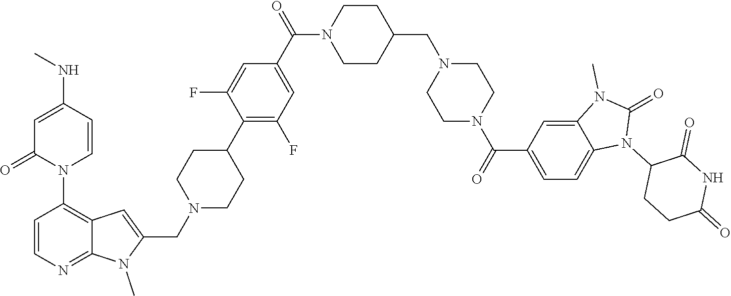 [SMILES: CNc1ccn(-c2ccnc3c2cc(CN2CCC(c4c(F)cc(C(=O)N5CCC(CN6CCN(C(=O)c7ccc8c(c7)n(C)c(=O)n8C7CCC(=O)NC7=O)CC6)CC5)cc4F)CC2)n3C)c(=O)c1]

### Example 3u264. 3-(5-(4-((1-(3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)methyl)piperazine-1-carbonyl)-3-methyl-2-oxo-2,3-dihydro-1H-benzo[d]imidazol-1-yl)piperidine-2,6-dione

Example 3u264 (22 mg) was prepared similarly to Example 3u163 from Ints. 2G108/2T59 (0.10 g/0.15 g).

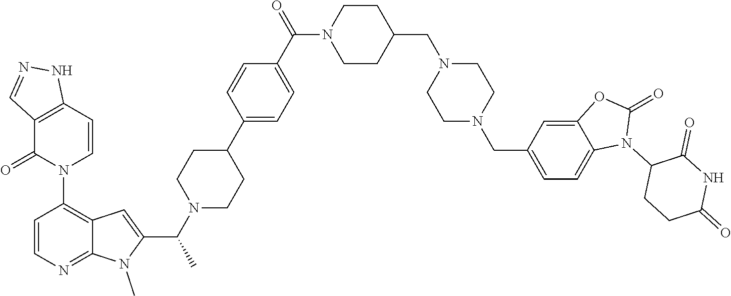 [SMILES: C[C@H](c1cc2c(-n3ccc4[nH]ncc4c3=O)ccnc2n1C)N1CCC(c2ccc(C(=O)N3CCC(CN4CCN(Cc5ccc6c(c5)oc(=O)n6C5CCC(=O)NC5=O)CC4)CC3)cc2)CC1]

### Example 3u265. 3-(6-((4-((1-(4-(1-((R)-1-(1-methyl-4-(4-oxo-1,4-dihydro-5H-pyrazolo[4,3-c]pyridin-5-yl)-1H-pyrrolo[2,3-b]pyridin-2-yl)ethyl)piperidin-4-yl)benzoyl)piperidin-4-yl)methyl)piperazin-1-yl)methyl)-2-oxobenzo[d]oxazol-3 (2H)-yl)piperidine-2,6-dione

Example 3u265 (36 mg) was prepared similarly to Example 3u163 from Ints. 2T77/2T57 (0.10 g/0.13 g).

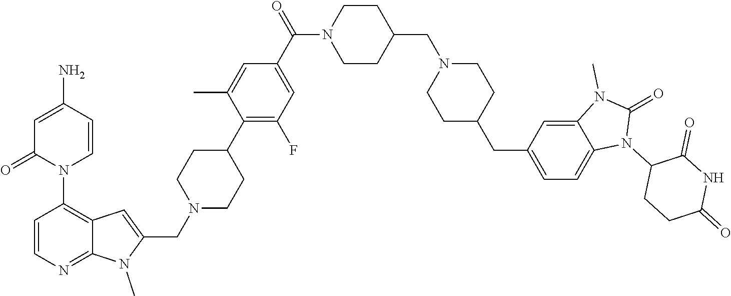 [SMILES: Cc1cc(C(=O)N2CCC(CN3CCC(Cc4ccc5c(c4)n(C)c(=O)n5C4CCC(=O)NC4=O)CC3)CC2)cc(F)c1C1CCN(Cc2cc3c(-n4ccc(N)cc4=O)ccnc3n2C)CC1]

### Example 3u267. 3-(5-((1-((1-(4-(1-((4-(4-amino-2-oxopyridin-1 (2H)-yl)-1-methyl-1H-pyrrolo[2,3-b]pyridin-2-yl)methyl)piperidin-4-yl)-3-fluoro-5-methylbenzoyl)piperidin-4-yl)methyl)piperidin-4-yl)methyl)-3-methyl-2-oxo-2,3-dihydro-1H-benzo[d]imidazol-1-yl)piperidine-2,6-dione

Example 3u267 (25 mg) was prepared similarly to Example 3u163 from Ints. 2T107/2T108 (20 mg/26 mg).

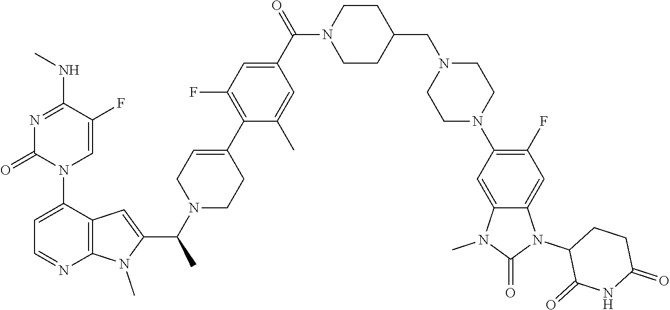 [SMILES: CNc1nc(=O)n(-c2ccnc3c2cc([C@H](C)N2CC=C(c4c(C)cc(C(=O)N5CCC(CN6CCN(c7cc8c(cc7F)n(C7CCC(=O)NC7=O)c(=O)n8C)CC6)CC5)cc4F)CC2)n3C)cc1F]

### Example 3u268. 3-(6-fluoro-5-(4-((1-(3-fluoro-4-(1-((S)-1-(4-(5-fluoro-4-(methylamino)-2-oxopyrimidin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)-3-methyl-2-oxo-2,3-dihydro-1H-benzo[d]-imidazol-1-yl)piperidine-2,6-dione

Example 3u268 (25 mg) was prepared similarly to Example 3u163 from Ints. 2G20/2T37 (80 mg/89 mg).

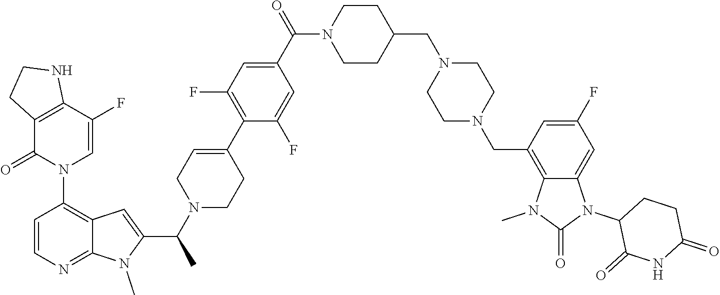 [SMILES: C[C@@H](c1cc2c(-n3cc(F)c4c(c3=O)CCN4)ccnc2n1C)N1CC=C(c2c(F)cc(C(=O)N3CCC(CN4CCN(Cc5cc(F)cc6c5n(C)c(=O)n6C5CCC(=O)NC5=O)CC4)CC3)cc2F)CC1]

### Example 3u269. 3-(5-(4-((1-(3,5-difluoro-4-(1-((S)-1-(4-(7-fluoro-4-oxo-1,2,3,4-tetrahydro-5H-pyrrolo[3,2-c]pyridin-5-yl)-1-methyl-1H-pyrrolo[2,3-b]pyridin-2-yl)ethyl)-1,2,3,6-tetrahydropyridin-4-yl)benzoyl)piperidin-4-yl)methyl)piperazin-1-yl)-6-fluoro-3-methyl-2-oxo-2,3-dihydro-1H-benzo[d]-imidazol-1-yl)piperidine-2,6-dione

Example 3u269 (25 mg) was prepared similarly to Example 3u163 from Ints. 2G11/2T37 (0.10 g/0.13 g).

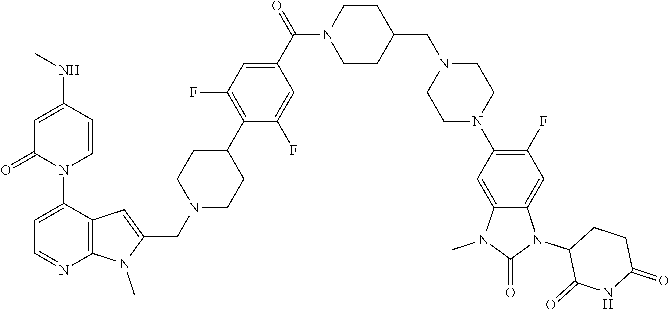 [SMILES: CNc1ccn(-c2ccnc3c2cc(CN2CCC(c4c(F)cc(C(=O)N5CCC(CN6CCN(c7cc8c(cc7F)n(C7CCC(=O)NC7=O)c(=O)n8C)CC6)CC5)cc4F)CC2)n3C)c(=O)c1]

### Example 3u270. 3-(5-(4-((1-(3,5-difluoro-4-(1-((1-methyl-4-(4-(methylamino)-2-oxopyridin-1 (2H)-yl)-1H-pyrrolo[2,3-b]pyridin-2-yl)methyl)piperidin-4-yl)benzoyl)piperidin-4-yl)methyl)piperazin-1-yl)-6-fluoro-3-methyl-2-oxo-2,3-dihydro-1H-benzo[d]imidazol-1-yl)piperidine-2,6-dione

Example 3u270 (16 mg) was prepared similarly to Example 3u163 from Ints. 2G108/2T37 (0.12 g/0.15 g).

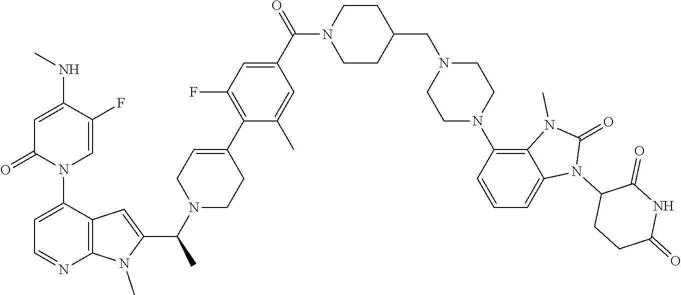 [SMILES: CNc1cc(=O)n(-c2ccnc3c2cc([C@H](C)N2CC=C(c4c(C)cc(C(=O)N5CCC(CN6CCN(c7cccc8c7n(C)c(=O)n8C7CCC(=O)NC7=O)CC6)CC5)cc4F)CC2)n3C)cc1F]

### Example 3u271. 3-(4-(4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)-piperidin-4-yl)methyl)piperazin-1-yl)-3-methyl-2-oxo-2,3-dihydro-1H-benzo[d]imidazol-1-yl)-piperidine-2,6-dione

Example 3u271 (20 mg) was prepared similarly to Example 3u163 from Ints. 2G27/2T112 (0.12 g/0.14 g).

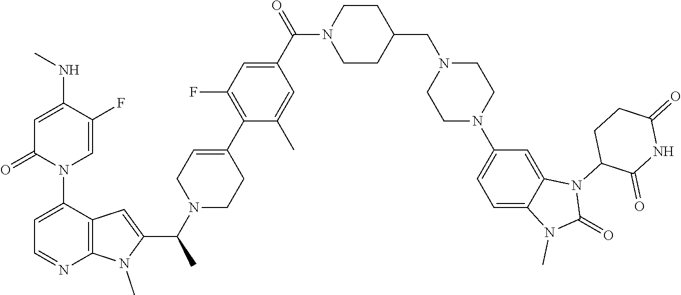 [SMILES: CNc1cc(=O)n(-c2ccnc3c2cc([C@H](C)N2CC=C(c4c(C)cc(C(=O)N5CCC(CN6CCN(c7ccc8c(c7)n(C7CCC(=O)NC7=O)c(=O)n8C)CC6)CC5)cc4F)CC2)n3C)cc1F]

### Example 3u272. 3-(6-(4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)-3-methyl-2-oxo-2,3-dihydro-1H-benzo[d]imidazol-1-yl)piperidine-2,6-dione

Example 3u272 (45 mg) was prepared similarly to Example 3u163 from Ints. 2G45/2T115 (0.10 g/0.14 g).

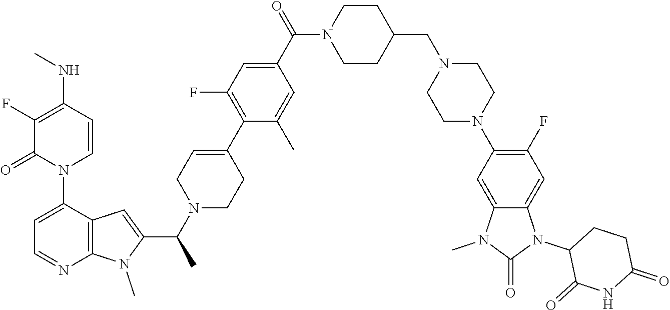 [SMILES: CNc1ccn(-c2ccnc3c2cc([C@H](C)N2CC=C(c4c(C)cc(C(=O)N5CCC(CN6CCN(c7cc8c(cc7F)n(C7CCC(=O)NC7=O)c(=O)n8C)CC6)CC5)cc4F)CC2)n3C)c(=O)c1F]

### Example 3u273. 3-(6-fluoro-5-(4-((1-(3-fluoro-4-(1-((S)-1-(4-(3-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)-3-methyl-2-oxo-2,3-dihydro-1H-benzo[d]imidazol-1-yl)piperidine-2,6-dione

Example 3u273 (15 mg) was prepared similarly to Example 3u163 from Ints. 3U39/2T37 (0.11 g/0.14 g).

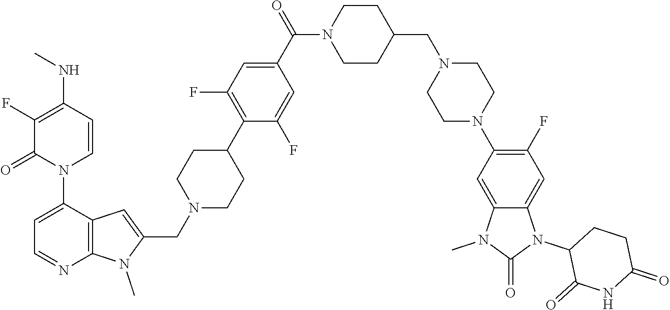 [SMILES: CNc1ccn(-c2ccnc3c2cc(CN2CCC(c4c(F)cc(C(=O)N5CCC(CN6CCN(c7cc8c(cc7F)n(C7CCC(=O)NC7=O)c(=O)n8C)CC6)CC5)cc4F)CC2)n3C)c(=O)c1F]

### Example 3u274. 3-(5-(4-((1-(3,5-difluoro-4-(1-((4-(3-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)methyl)piperazin-1-yl)-6-fluoro-3-methyl-2-oxo-2,3-dihydro-1H-benzo[d]imidazol-1-yl)piperidine-2,6-dione

Example 3u274 (29 mg) was prepared similarly to Example 3u163 from Ints. 3U78/2T73 (0.10 g/0.14 g).

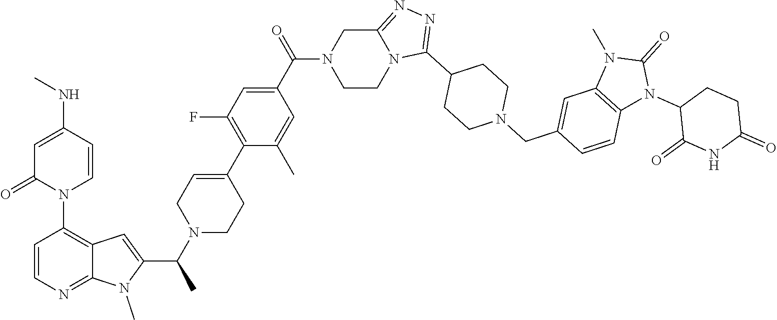 [SMILES: CNc1ccn(-c2ccnc3c2cc([C@H](C)N2CC=C(c4c(C)cc(C(=O)N5CCn6c(nnc6C6CCN(Cc7ccc8c(c7)n(C)c(=O)n8C7CCC(=O)NC7=O)CC6)C5)cc4F)CC2)n3C)c(=O)c1]

### Example 3u275. 3-(5-((4-(7-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)-5,6,7,8-tetrahydro-[1,2,4]triazolo[4,3-a]pyrazin-3-yl)piperidin-1-yl)methyl)-3-methyl-2-oxo-2,3-dihydro-1H-benzo[d]imidazol-1-yl)piperidine-2,6-dione

Ints. 2T118/2G46 (34 mg/0.37 g) and HATU (0.37 g) were stirred ON in DMF/DIPEA (1 mL/0.55 mL). The residue after concentration was HPLC-purified to give Example 3u275 (12 mg).

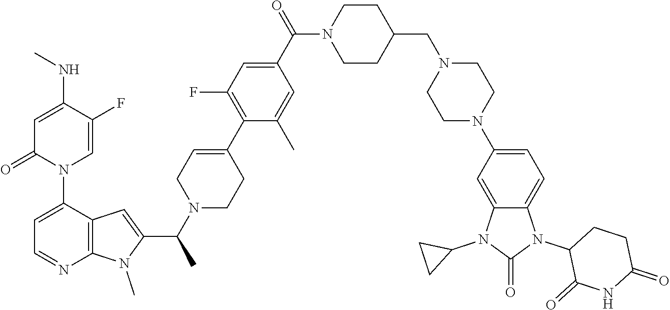 [SMILES: CNc1cc(=O)n(-c2ccnc3c2cc([C@H](C)N2CC=C(c4c(C)cc(C(=O)N5CCC(CN6CCN(c7ccc8c(c7)n(C7CC7)c(=O)n8C7CCC(=O)NC7=O)CC6)CC5)cc4F)CC2)n3C)cc1F]

### Int. 3u276. 3-(3-cyclopropyl-5-(4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methyl-benzoyl)piperidin-4-yl)methyl)piperazin-1-yl)-2-oxo-2,3-dihydro-1H-benzo[d]imidazol-1-yl)piperidine-2,6-dione

Example 3u276 (20 mg) was prepared similarly to Example 3u163 from Ints. 2G45/2T86 (0.10 g/0.13 g).

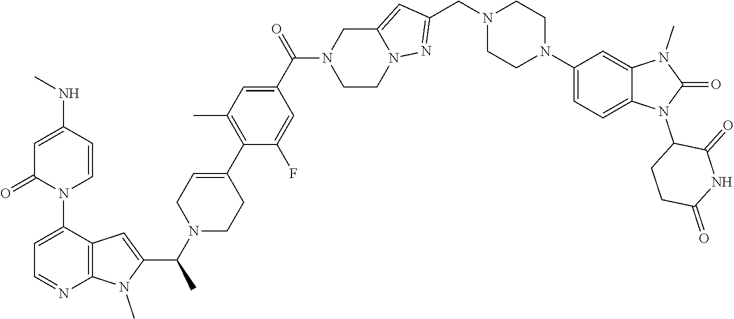 [SMILES: CNc1ccn(-c2ccnc3c2cc([C@H](C)N2CC=C(c4c(C)cc(C(=O)N5CCn6nc(CN7CCN(c8ccc9c(c8)n(C)c(=O)n9C8CCC(=O)NC8=O)CC7)cc6C5)cc4F)CC2)n3C)c(=O)c1]

### Example 3u277. 3-(5-(4-((5-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)-4,5,6,7-tetrahydropyrazolo[1,5-a]pyrazin-2-yl)methyl)piperazin-1-yl)-3-methyl-2-oxo-2,3-dihydro-1H-benzo[d]imidazol-1-yl)piperidine-2,6-dione

Int. 2G46 (62 mg) and HATU (48 mg) were stirred 10 min in DMF/DIPEA (5 mL/0.04 mL). Int. 120 (58 mg) was added and stirring continued 15 h. The residue after concentration was HPLC-purified to give Example 3u277 (11 mg).

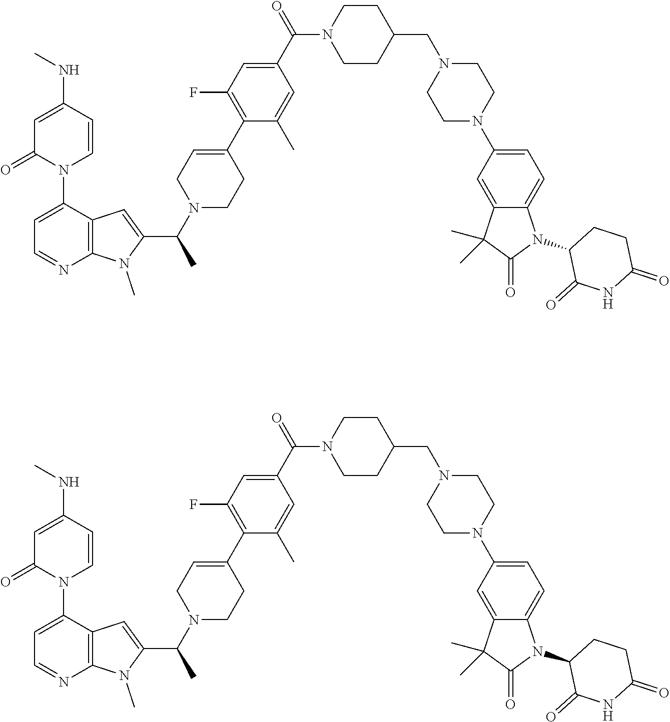 [SMILES: CNc1ccn(-c2ccnc3c2cc([C@H](C)N2CC=C(c4c(C)cc(C(=O)N5CCC(CN6CCN(c7ccc8c(c7)C(C)(C)C(=O)N8[C@@H]7CCC(=O)NC7=O)CC6)CC5)cc4F)CC2)n3C)c(=O)c1 ; CNc1ccn(-c2ccnc3c2cc([C@H](C)N2CC=C(c4c(C)cc(C(=O)N5CCC(CN6CCN(c7ccc8c(c7)C(C)(C)C(=O)N8[C@H]7CCC(=O)NC7=O)CC6)CC5)cc4F)CC2)n3C)c(=O)c1]

### Example 3u281 and 3u280. (R)-3-(5-(4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)-3,3-dimethyl-2-oxoindolin-1-yl)piperidine-2,6-dione and (S)-3-(5-(4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)-3,3-dimethyl-2-oxoindolin-1-yl)piperidine-2,6-dione

Example 3u304 (0.14 g) was resolved by HPLC using a Chiralpak IC 250×10 mm 5 μm column with an eluent of 35% ACN, 35% IPA and 30% DCM (52 mL/min) to give Example 3u281 (28 mg, first peak) and Example 3u280 (26 mg, second peak) after a subsequent HPLC-purification. The absolute configurations were not determined at the glutarimide chiral centers for these compounds.

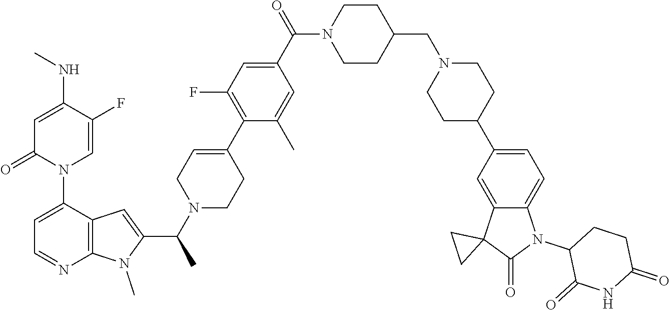 [SMILES: CNc1cc(=O)n(-c2ccnc3c2cc([C@H](C)N2CC=C(c4c(C)cc(C(=O)N5CCC(CN6CCC(c7ccc8c(c7)C7(CC7)C(=O)N8C7CCC(=O)NC7=O)CC6)CC5)cc4F)CC2)n3C)cc1F]

### Example 3u282. 3-(5′-(1-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)-piperidin-4-yl)methyl)piperidin-4-yl)-2′-oxospiro[cyclopropane-1,3′-indolin]-1′-yl)piperidine-2,6-dione

Example 3u282 (24 mg) was prepared similarly to Example 3u163 from Ints. 2G45/2T94 (0.10 g/0.14 g).

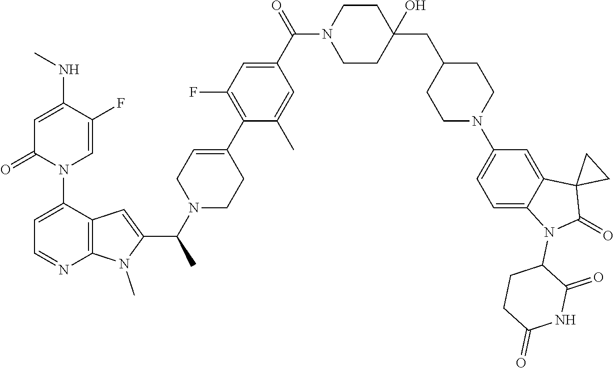 [SMILES: CNc1cc(=O)n(-c2ccnc3c2cc([C@H](C)N2CC=C(c4c(C)cc(C(=O)N5CCC(O)(CC6CCN(c7ccc8c(c7)C7(CC7)C(=O)N8C7CCC(=O)NC7=O)CC6)CC5)cc4F)CC2)n3C)cc1F]

### Example 3u283. 3-(5′-(4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)-4-hydroxypiperidin-4-yl)methyl)piperidin-1-yl)-2′-oxospiro[cyclopropane-1,3′-indolin]-1′-yl)piperidine-2,6-dione

Example 3u283 (30 mg) was prepared similarly to Example 3u163 from Ints. 2G45/2T100 (0.10 g/0.16 g).

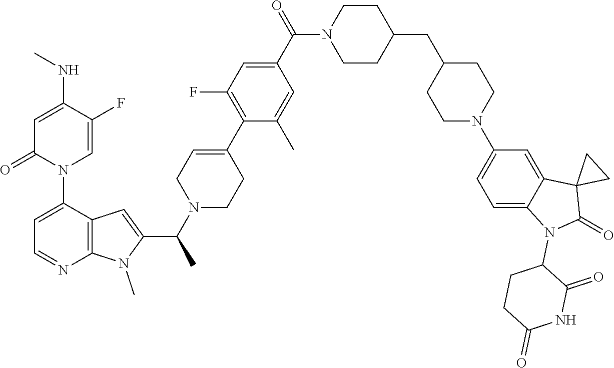 [SMILES: CNc1cc(=O)n(-c2ccnc3c2cc([C@H](C)N2CC=C(c4c(C)cc(C(=O)N5CCC(CC6CCN(c7ccc8c(c7)C7(CC7)C(=O)N8C7CCC(=O)NC7=O)CC6)CC5)cc4F)CC2)n3C)cc1F]

### Example 3u284. 3-(5′-(4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperidin-1-yl)-2′-oxospiro[cyclopropane-1,3′-indolin]-1′-yl)piperidine-2,6-dione

Example 3u284 (30 mg) was prepared similarly to Example 3u163 from Ints. 2G45/3C1 (80 mg/0.11 g).

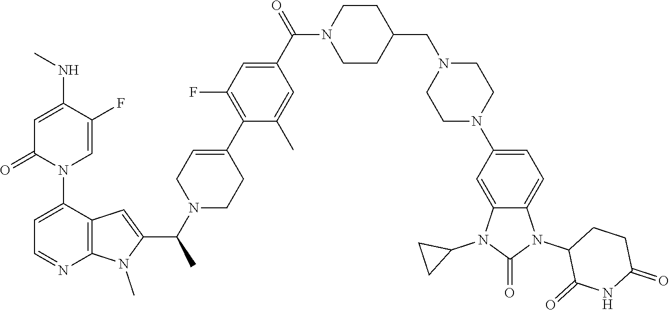 [SMILES: CNc1cc(=O)n(-c2ccnc3c2cc([C@H](C)N2CC=C(c4c(C)cc(C(=O)N5CCC(CN6CCN(c7ccc8c(c7)n(C7CC7)c(=O)n8C7CCC(=O)NC7=O)CC6)CC5)cc4F)CC2)n3C)cc1F]

### Example 3u287. 3-(3-cyclopropyl-5-(4-((1-(3-fluoro-5-methyl-4-(1-((S)-1-(1-methyl-4-(4-(methyl-amino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)-2-oxo-2,3-dihydro-1H-benzo[d]imidazol-1-yl)-piperidine-2,6-dione

Example 3u287 (0.12 g) was prepared similarly to Example 3u163 from Ints. 2G46/2T86 (0.30 g/0.35 g).

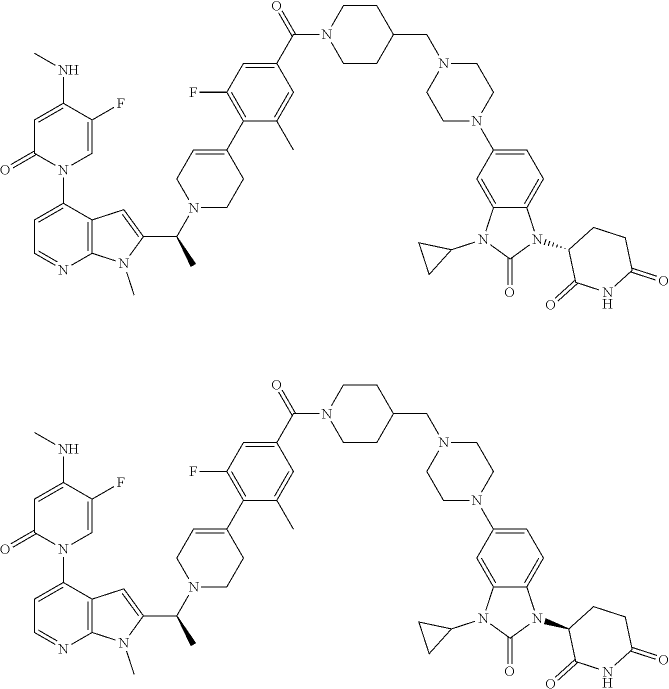 [SMILES: CNc1cc(=O)n(-c2ccnc3c2cc([C@H](C)N2CC=C(c4c(C)cc(C(=O)N5CCC(CN6CCN(c7ccc8c(c7)n(C7CC7)c(=O)n8[C@@H]7CCC(=O)NC7=O)CC6)CC5)cc4F)CC2)n3C)cc1F ; CNc1cc(=O)n(-c2ccnc3c2cc([C@H](C)N2CC=C(c4c(C)cc(C(=O)N5CCC(CN6CCN(c7ccc8c(c7)n(C7CC7)c(=O)n8[C@H]7CCC(=O)NC7=O)CC6)CC5)cc4F)CC2)n3C)cc1F]

### Example 3u279 and 3u278. (R)-3-(3-cyclopropyl-5-(4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)-2-oxo-2,3-dihydro-1H-benzo[d]imidazol-1-yl)piperidine-2,6-dione and (S)-3-(3-cyclopropyl-5-(4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)-2-oxo-2,3-dihydro-1H-benzo[d]imidazol-1-yl)piperidine-2,6-dione

Example 3u287 (0.13 g) was resolved by HPLC using a CELLULOSE-SZ 250×30 mm 5 μm column with an eluent of 35% ACN, 35% IPA and 30% DCM (42 mL/min) to give Example 3u279 (45 mg, first peak) and Example 3u278 (43 mg, second peak). The absolute configurations were not determined at the glutarimide chiral centers for these compounds.

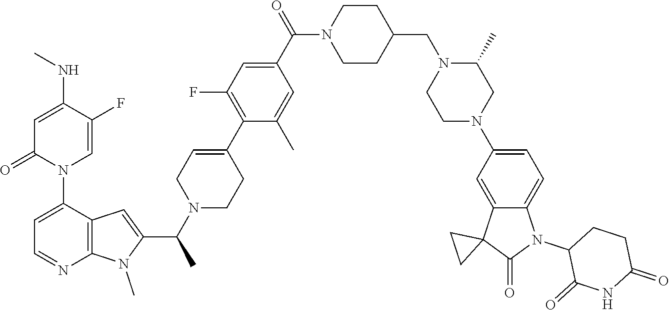 [SMILES: CNc1cc(=O)n(-c2ccnc3c2cc([C@H](C)N2CC=C(c4c(C)cc(C(=O)N5CCC(CN6CCN(c7ccc8c(c7)C7(CC7)C(=O)N8C7CCC(=O)NC7=O)C[C@H]6C)CC5)cc4F)CC2)n3C)cc1F]

### Example 3u295. 3-(5′-((R)-4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)-3-methylpiperazin-1-yl)-2′-oxospiro[cyclopropane-1,3′-indolin]-1′-yl)piperidine-2,6-dione

Example 3u295 (75 mg) was prepared similarly to Example 3u163 from Ints. 2G45/2T89 (0.28 g/0.40 g).

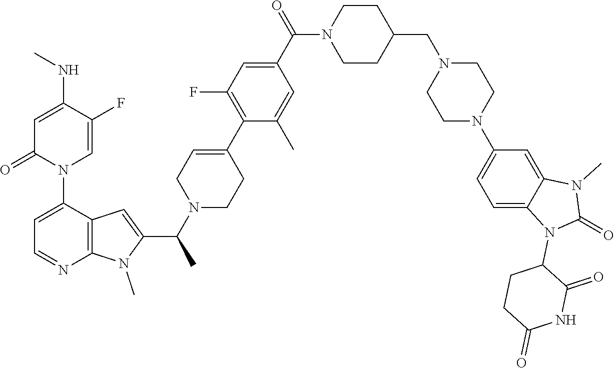 [SMILES: CNc1cc(=O)n(-c2ccnc3c2cc([C@H](C)N2CC=C(c4c(C)cc(C(=O)N5CCC(CN6CCN(c7ccc8c(c7)n(C)c(=O)n8C7CCC(=O)NC7=O)CC6)CC5)cc4F)CC2)n3C)cc1F]

### Example 3u298. 3-(5-(4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)-piperidin-4-yl)methyl)piperazin-1-yl)-3-methyl-2-oxo-2,3-dihydro-1H-benzo[d]imidazol-1-yl)-piperidine-2,6-dione

Example 3u298 (0.16 g) was prepared similarly to Example 3u163 from Ints. 2G45/2T101 (0.25 g/0.31 g).

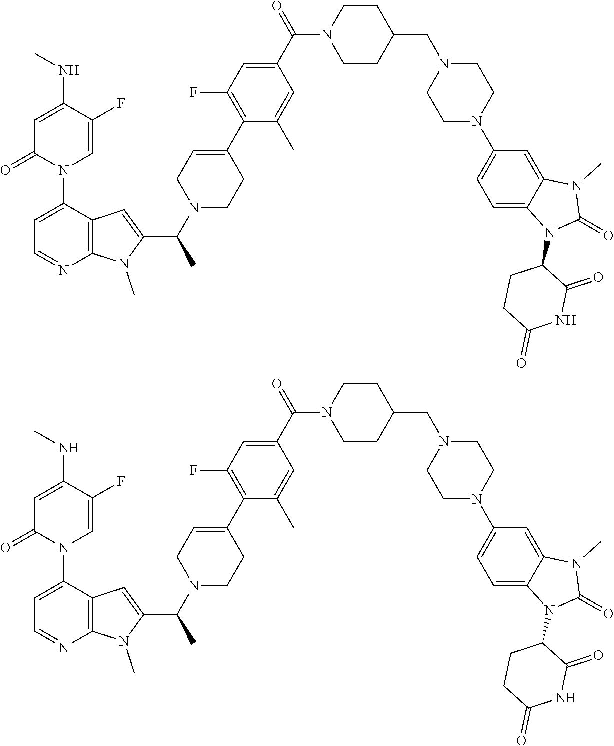 [SMILES: CNc1cc(=O)n(-c2ccnc3c2cc([C@H](C)N2CC=C(c4c(C)cc(C(=O)N5CCC(CN6CCN(c7ccc8c(c7)n(C)c(=O)n8[C@@H]7CCC(=O)NC7=O)CC6)CC5)cc4F)CC2)n3C)cc1F ; CNc1cc(=O)n(-c2ccnc3c2cc([C@H](C)N2CC=C(c4c(C)cc(C(=O)N5CCC(CN6CCN(c7ccc8c(c7)n(C)c(=O)n8[C@H]7CCC(=O)NC7=O)CC6)CC5)cc4F)CC2)n3C)cc1F]

### Example 3u297 and 3u296. (R)-3-(5-(4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)-3-methyl-2-oxo-2,3-dihydro-1H-benzo[d]-imidazol-1-yl)piperidine-2,6-dione and (S)-3-(5-(4-((1-(3-fluoro-4-(1-((S)-1-(4-(5-fluoro-4-(methyl-amino)-2-oxopyridin-1 (2H)-yl)-1-methyl-1H-pyrrolo[2,3-b]pyridin-2-yl)ethyl)-1,2,3,6-tetrahydro-pyridin-4-yl)-5-methylbenzoyl)piperidin-4-yl)methyl)piperazin-1-yl)-3-methyl-2-oxo-2,3-dihydro-1H-benzo[d]imidazol-1-yl)piperidine-2,6-dione

Example 3u298 (0.10 g) was resolved by HPLC using a CELLULOSE-SZ 250×10 mm 5 μm column with an eluent of 35% ACN, 35% IPA and 30% DCM (4 mL/min) to give Example 3u297 (35 mg, first peak) and Example 3u296 (32 mg, second peak). The absolute configurations were not determined at the glutarimide chiral centers for these compounds.

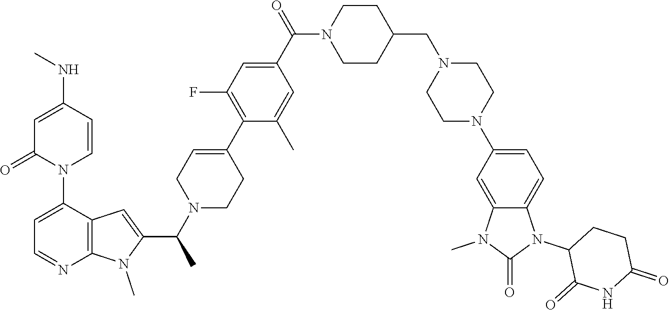 [SMILES: CNc1ccn(-c2ccnc3c2cc([C@H](C)N2CC=C(c4c(C)cc(C(=O)N5CCC(CN6CCN(c7ccc8c(c7)n(C)c(=O)n8C7CCC(=O)NC7=O)CC6)CC5)cc4F)CC2)n3C)c(=O)c1]

### Example 3u299. 3-(5-(4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)-3-methyl-2-oxo-2,3-dihydro-1H-benzo[d]imidazol-1-yl)piperidine-2,6-dione

Example 3u299 (0.17 g) was prepared similarly to Example 3u163 from Ints. 2G46/2T101 (0.25 g/0.32 g).

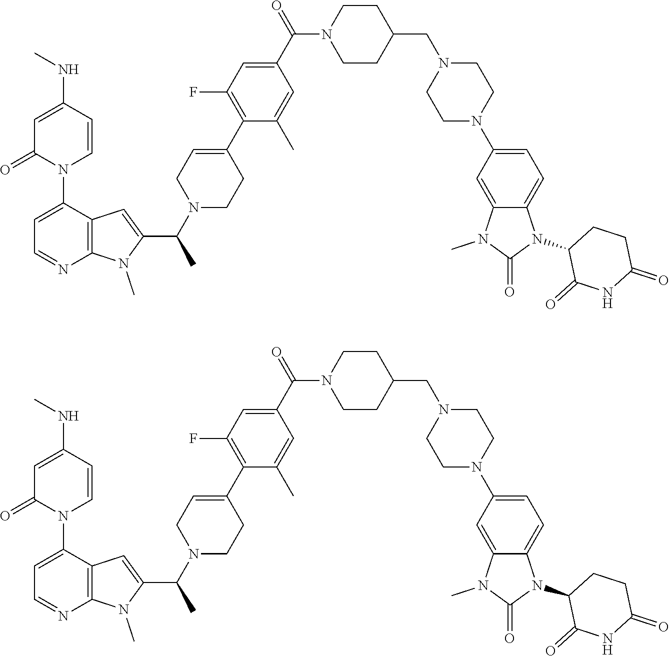 [SMILES: CNc1ccn(-c2ccnc3c2cc([C@H](C)N2CC=C(c4c(C)cc(C(=O)N5CCC(CN6CCN(c7ccc8c(c7)n(C)c(=O)n8[C@@H]7CCC(=O)NC7=O)CC6)CC5)cc4F)CC2)n3C)c(=O)c1 ; CNc1ccn(-c2ccnc3c2cc([C@H](C)N2CC=C(c4c(C)cc(C(=O)N5CCC(CN6CCN(c7ccc8c(c7)n(C)c(=O)n8[C@H]7CCC(=O)NC7=O)CC6)CC5)cc4F)CC2)n3C)c(=O)c1]

### Example 3u294 and 3u293. (R)-3-(5-(4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)-3-methyl-2-oxo-2,3-dihydro-1H-benzo[d]imidazol-1-yl)piperidine-2,6-dione and (S)-3-(5-(4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)-3-methyl-2-oxo-2,3-dihydro-1H-benzo[d]imidazol-1-yl)piperidine-2,6-dione

Example 3u299 (0.17 g) was resolved by HPLC using a CELLULOSE-SZ 250×10 mm 5 μm column with an eluent of 40% ACN, 40% IPA and 20% DCM (6 mL/min) to give Example 3u294 (60 mg, first peak) and Example 3u293 (69 mg, second peak). The absolute configurations were not determined at the glutarimide chiral centers for these compounds.

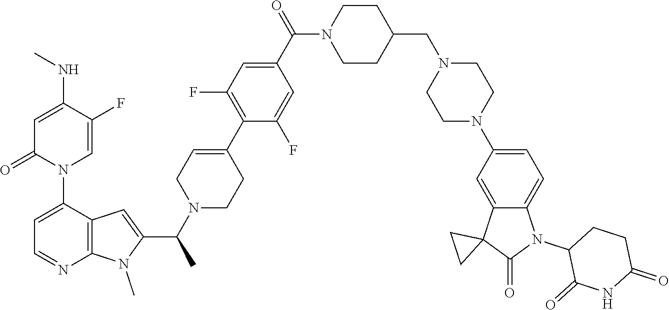 [SMILES: CNc1cc(=O)n(-c2ccnc3c2cc([C@H](C)N2CC=C(c4c(F)cc(C(=O)N5CCC(CN6CCN(c7ccc8c(c7)C7(CC7)C(=O)N8C7CCC(=O)NC7=O)CC6)CC5)cc4F)CC2)n3C)cc1F]

### Example 3u300. 3-(5′-(4-((1-(3,5-difluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)benzoyl)-piperidin-4-yl)methyl)piperazin-1-yl)-2′-oxospiro[cyclopropane-1,3′-indolin]-1′-yl)piperidine-2,6-dione

Example 3u300 (0.30 g) was prepared similarly to Example 3u163 from Ints. 2G74/3U1 (0.35 g/0.48 g).

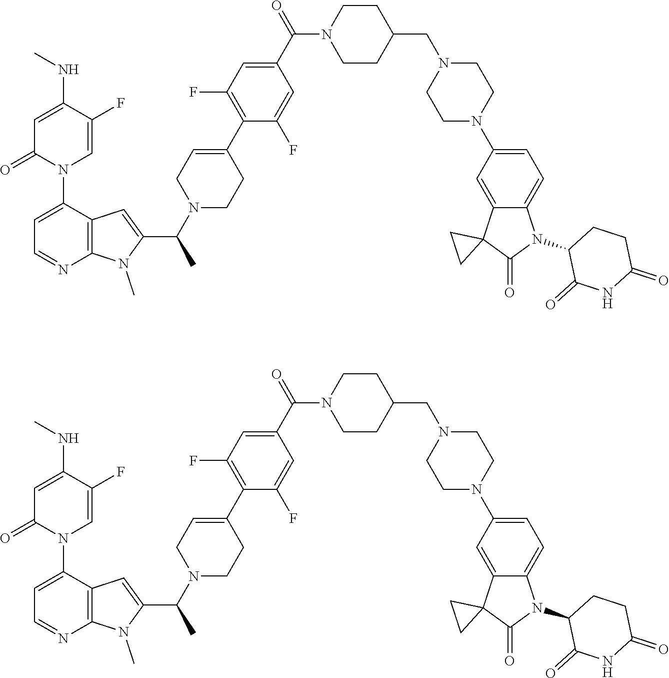 [SMILES: CNc1cc(=O)n(-c2ccnc3c2cc([C@H](C)N2CC=C(c4c(F)cc(C(=O)N5CCC(CN6CCN(c7ccc8c(c7)C7(CC7)C(=O)N8[C@@H]7CCC(=O)NC7=O)CC6)CC5)cc4F)CC2)n3C)cc1F ; CNc1cc(=O)n(-c2ccnc3c2cc([C@H](C)N2CC=C(c4c(F)cc(C(=O)N5CCC(CN6CCN(c7ccc8c(c7)C7(CC7)C(=O)N8[C@H]7CCC(=O)NC7=O)CC6)CC5)cc4F)CC2)n3C)cc1F]

### Example 3u290. (R)-3-(5′-(4-((1-(3,5-difluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)benzoyl)piperidin-4-yl)methyl)piperazin-1-yl)-2′-oxospiro[cyclopropane-1,3′-indolin]-1′-yl)piperidine-2,6-dione or (S)-3-(5′-(4-((1-(3,5-difluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)benzoyl)piperidin-4-yl)methyl)piperazin-1-yl)-2′-oxospiro[cyclopropane-1,3′-indolin]-1′-yl)piperidine-2,6-dione

Example 3u300 (0.24 g) was resolved by HPLC using a Chiralpak IK 250×30 mm 5 μm column with an eluent of 20% ACN, 40% IPA and 40% DCM (36 mL/min) to give Example 2u290 (27 mg, first peak). The second eluting peak was resolved by HPLC using a Chiralpak IC 250×30 mm 5 μm column with an eluent of 35% ACN, 35% IPA and 30% DCM (52 mL/min) to give the other diastereomer (10 mg, second peak). The absolute configurations were not determined at the glutarimide chiral centers for these compounds.

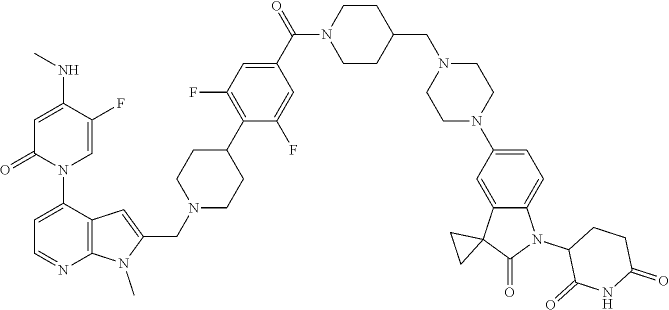 [SMILES: CNc1cc(=O)n(-c2ccnc3c2cc(CN2CCC(c4c(F)cc(C(=O)N5CCC(CN6CCN(c7ccc8c(c7)C7(CC7)C(=O)N8C7CCC(=O)NC7=O)CC6)CC5)cc4F)CC2)n3C)cc1F]

### Example 3u301. 3-(5′-(4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)methyl)-piperazin-1-yl)-2′-oxospiro[cyclopropane-1,3′-indolin]-1′-yl)piperidine-2,6-dione

Example 3u301 (0.23 g) was prepared similarly to Example 3u163 from Ints. 2G58/3U1 (0.30 g/0.42 g).

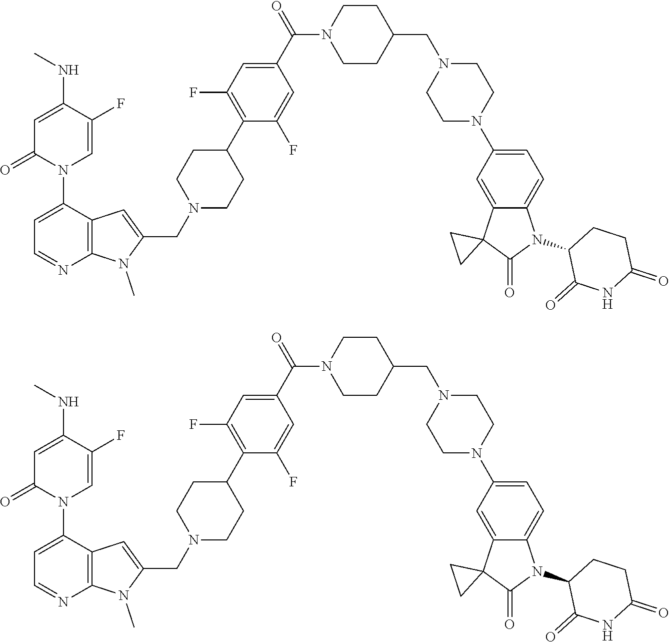 [SMILES: CNc1cc(=O)n(-c2ccnc3c2cc(CN2CCC(c4c(F)cc(C(=O)N5CCC(CN6CCN(c7ccc8c(c7)C7(CC7)C(=O)N8[C@@H]7CCC(=O)NC7=O)CC6)CC5)cc4F)CC2)n3C)cc1F ; CNc1cc(=O)n(-c2ccnc3c2cc(CN2CCC(c4c(F)cc(C(=O)N5CCC(CN6CCN(c7ccc8c(c7)C7(CC7)C(=O)N8[C@H]7CCC(=O)NC7=O)CC6)CC5)cc4F)CC2)n3C)cc1F]

### Example 3u289 and 3u288. (R)-3-(5′-(4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)methyl)piperazin-1-yl)-2′-oxospiro[cyclopropane-1,3′-indolin]-1′-yl)piperidine-2,6-dione and (S)-3-(5′-(4-((1-(3,5-difluoro-4-(1-((4-(5-fluoro-4-(methylamino)-2-oxopyridin-1 (2H)-yl)-1-methyl-1H-pyrrolo[2,3-b]pyridin-2-yl)methyl)piperidin-4-yl)benzoyl)piperidin-4-yl)methyl)piperazin-1-yl)-2′-oxospiro[cyclopropane-1,3′-indolin]-1′-yl)piperidine-2,6-dione

Example 3u301 (0.21 g) was resolved by HPLC using a CELLULOSE-SZ 250×30 mm 5 μm column with an eluent of 40% ACN, 40% IPA and 20% DCM (36 mL/min) to give Example 3u289 (70 mg, first peak) and Example 3u288 (56 mg, second peak). The absolute configurations were not determined at the glutarimide chiral centers for these compounds.

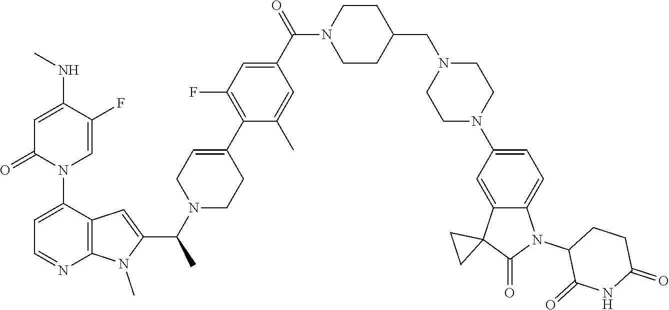 [SMILES: CNc1cc(=O)n(-c2ccnc3c2cc([C@H](C)N2CC=C(c4c(C)cc(C(=O)N5CCC(CN6CCN(c7ccc8c(c7)C7(CC7)C(=O)N8C7CCC(=O)NC7=O)CC6)CC5)cc4F)CC2)n3C)cc1F]

### Example 3u302. 3-(5′-(4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)-2′-oxospiro[cyclopropane-1,3′-indolin]-1′-yl)piperidine-2,6-dione

Example 3u302 (70 mg) was prepared similarly to Example 3u163 from Ints. 2G45/3U1 (0.15 g/0.21 g).

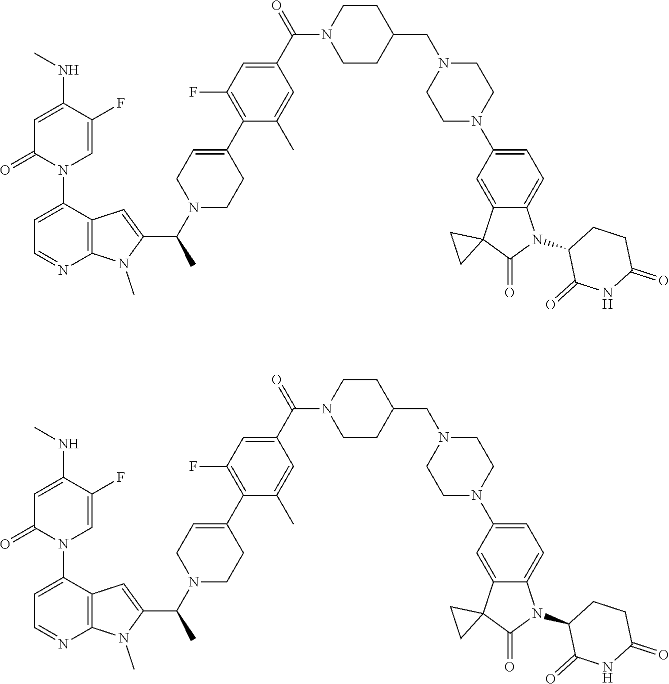 [SMILES: CNc1cc(=O)n(-c2ccnc3c2cc([C@H](C)N2CC=C(c4c(C)cc(C(=O)N5CCC(CN6CCN(c7ccc8c(c7)C7(CC7)C(=O)N8[C@@H]7CCC(=O)NC7=O)CC6)CC5)cc4F)CC2)n3C)cc1F ; CNc1cc(=O)n(-c2ccnc3c2cc([C@H](C)N2CC=C(c4c(C)cc(C(=O)N5CCC(CN6CCN(c7ccc8c(c7)C7(CC7)C(=O)N8[C@H]7CCC(=O)NC7=O)CC6)CC5)cc4F)CC2)n3C)cc1F]

### Example 3u292 and 3u291. (R)-3-(5′-(4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)-2′-oxospiro[cyclopropane-1,3′-indolin]-1′-yl)piperidine-2,6-dione and (S)-3-(5′-(4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)-2′-oxospiro[cyclopropane-1,3′-indolin]-1′-yl)piperidine-2,6-dione

Example 3u302 (70 mg) was resolved by HPLC using a Chiralpak IK 250×30 mm 5 μm column with an eluent of 35% ACN, 35% IPA and 30% DCM (26 mL/min) to give Example 3u292 (7 mg, first peak) and Example 3u291 (5 mg, second peak). The absolute configurations were not determined at the glutarimide chiral centers for these compounds.

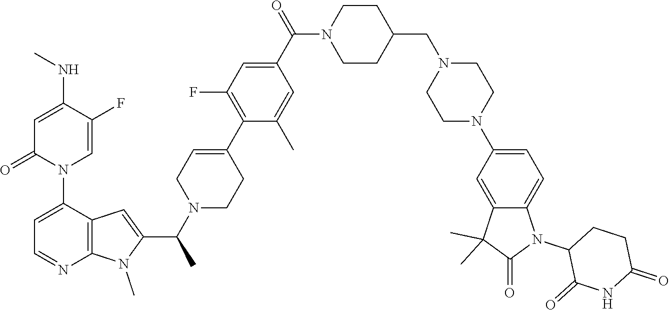 [SMILES: CNc1cc(=O)n(-c2ccnc3c2cc([C@H](C)N2CC=C(c4c(C)cc(C(=O)N5CCC(CN6CCN(c7ccc8c(c7)C(C)(C)C(=O)N8C7CCC(=O)NC7=O)CC6)CC5)cc4F)CC2)n3C)cc1F]

### Example 3u303. 3-(5-(4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)-3,3-dimethyl-2-oxoindolin-1-yl)piperidine-2,6-dione

Example 3u303 (0.11 g) was prepared similarly to Example 3u163 from Ints. 2G45/2T93 (0.26 g/0.33 g).

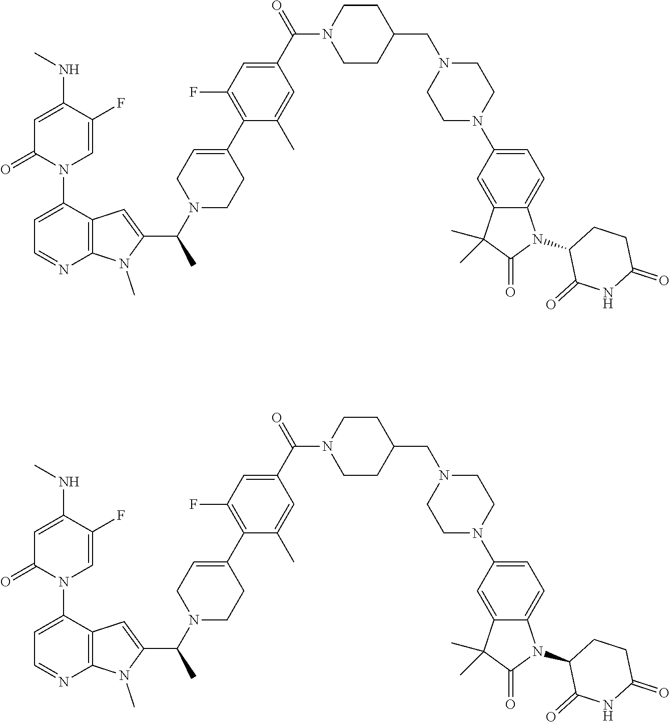 [SMILES: CNc1cc(=O)n(-c2ccnc3c2cc([C@H](C)N2CC=C(c4c(C)cc(C(=O)N5CCC(CN6CCN(c7ccc8c(c7)C(C)(C)C(=O)N8[C@@H]7CCC(=O)NC7=O)CC6)CC5)cc4F)CC2)n3C)cc1F ; CNc1cc(=O)n(-c2ccnc3c2cc([C@H](C)N2CC=C(c4c(C)cc(C(=O)N5CCC(CN6CCN(c7ccc8c(c7)C(C)(C)C(=O)N8[C@H]7CCC(=O)NC7=O)CC6)CC5)cc4F)CC2)n3C)cc1F]

### Example 3u286 and 3u285. (R)-3-(5-(4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)-3,3-dimethyl-2-oxoindolin-1-yl)piperidine-2,6-dione and (S)-3-(5-(4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)-3,3-dimethyl-2-oxoindolin-1-yl)piperidine-2,6-dione

Example 3u303 (0.19 g) was resolved by HPLC using a CELLULOSE-SC 250×30 mm 5 μm column with an eluent of 35% ACN, 35% IPA and 30% DCM (52 mL/min) to give Example 3u286 (24 mg, first peak) and Example 3u285 (20 mg, second peak). The absolute configurations were not determined at the glutarimide chiral centers for these compounds.

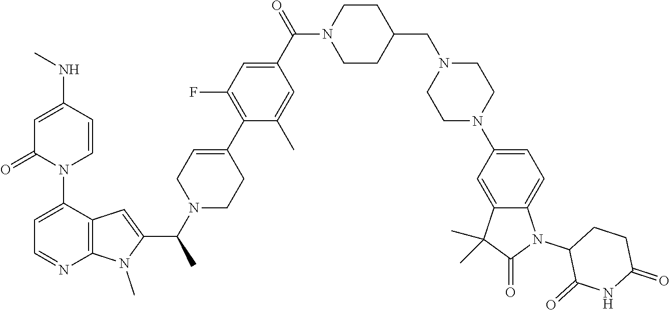 [SMILES: CNc1ccn(-c2ccnc3c2cc([C@H](C)N2CC=C(c4c(C)cc(C(=O)N5CCC(CN6CCN(c7ccc8c(c7)C(C)(C)C(=O)N8C7CCC(=O)NC7=O)CC6)CC5)cc4F)CC2)n3C)c(=O)c1]

### Example 3u304. 3-(5-(4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)-3,3-dimethyl-2-oxoindolin-1-yl)piperidine-2,6-dione

Example 3u304 (0.17 mg) was prepared similarly to Example 3u163 from Ints. 2G46/2T93 (0.30 g/0.40 g).

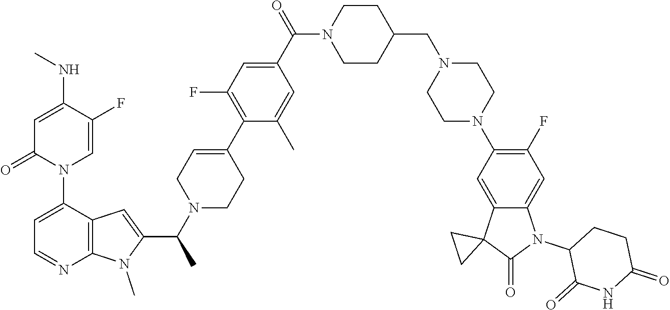 [SMILES: CNc1cc(=O)n(-c2ccnc3c2cc([C@H](C)N2CC=C(c4c(C)cc(C(=O)N5CCC(CN6CCN(c7cc8c(cc7F)N(C7CCC(=O)NC7=O)C(=O)C87CC7)CC6)CC5)cc4F)CC2)n3C)cc1F]

### Example 3u305. 3-(6′-fluoro-5′-(4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)-2′-oxospiro[cyclopropane-1,3′-indolin]-1′-yl)piperidine-2,6-dione

Example 3u305 (63 mg) was prepared similarly to Example 3u163 from Ints. 2G45/2T105 (0.12 g/0.17 g).

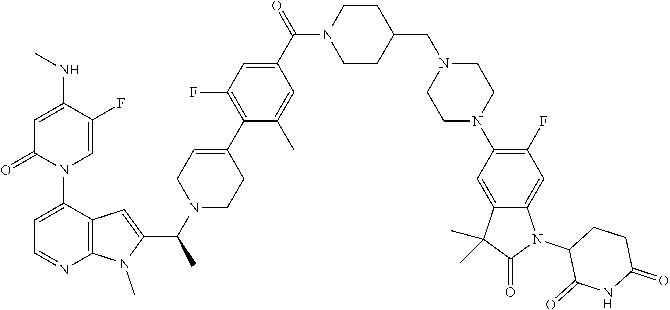 [SMILES: CNc1cc(=O)n(-c2ccnc3c2cc([C@H](C)N2CC=C(c4c(C)cc(C(=O)N5CCC(CN6CCN(c7cc8c(cc7F)N(C7CCC(=O)NC7=O)C(=O)C8(C)C)CC6)CC5)cc4F)CC2)n3C)cc1F]

### Example 3u306. 3-(6-fluoro-5-(4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)piperazin-1-yl)-3,3-dimethyl-2-oxoindolin-1-yl)piperidine-2,6-dione

Example 3u306 (14 mg) was prepared similarly to Example 3u163 from Ints. 2G45/TT73 (60 mg/74 mg).

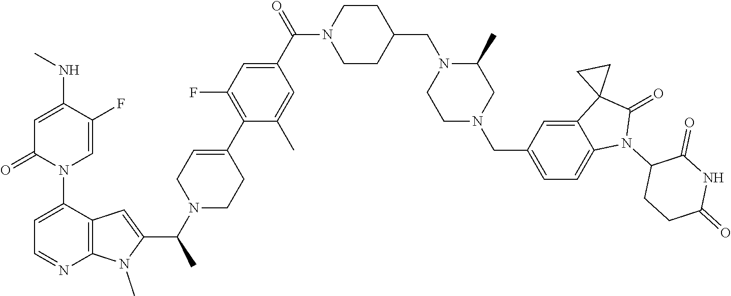 [SMILES: CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)cc1F]

### Example 3u309. 3-(5′-(((S)-4-((1-(3-fluoro-4-(1-((S)-1-(4-(5-fluoro-4-(methylamino)-2-oxopyridin-1 (2H)-yl)-1-methyl-1H-pyrrolo[2,3-b]pyridin-2-yl)ethyl)-1,2,3,6-tetrahydropyridin-4-yl)-5-methylbenzoyl)piperidin-4-yl)methyl)-3-methylpiperazin-1-yl)methyl)-2′-oxospiro[cyclobutane-1,3′-indolin]-1′-yl)piperidine-2,6-dione

Example 3u309 (36 mg) was prepared similarly to Example 3u163 from Ints. 2G45/3U30 (0.11 g/0.15 g).

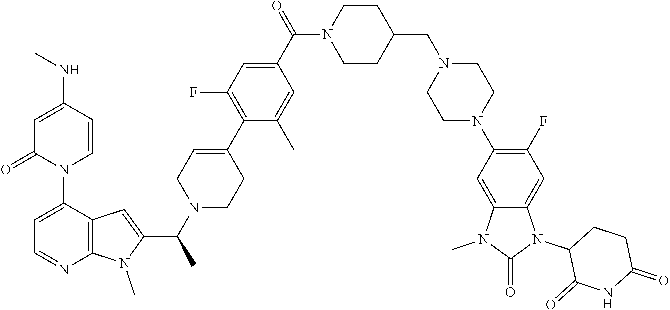 [SMILES: CNc1ccn(-c2ccnc3c2cc([C@H](C)N2CC=C(c4c(C)cc(C(=O)N5CCC(CN6CCN(c7cc8c(cc7F)n(C7CCC(=O)NC7=O)c(=O)n8C)CC6)CC5)cc4F)CC2)n3C)c(=O)c1]

### Example 3u310. 3-(6-fluoro-5-(4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)-piperidin-4-yl)methyl)piperazin-1-yl)-3-methyl-2-oxo-2,3-dihydro-1H-benzo[d]imidazol-1-yl)-piperidine-2,6-dione

Example 3u310 (0.21 g) was prepared similarly to Example 3u163 from Ints. 2G46-1/2T37 (0.30 g/0.35 g).

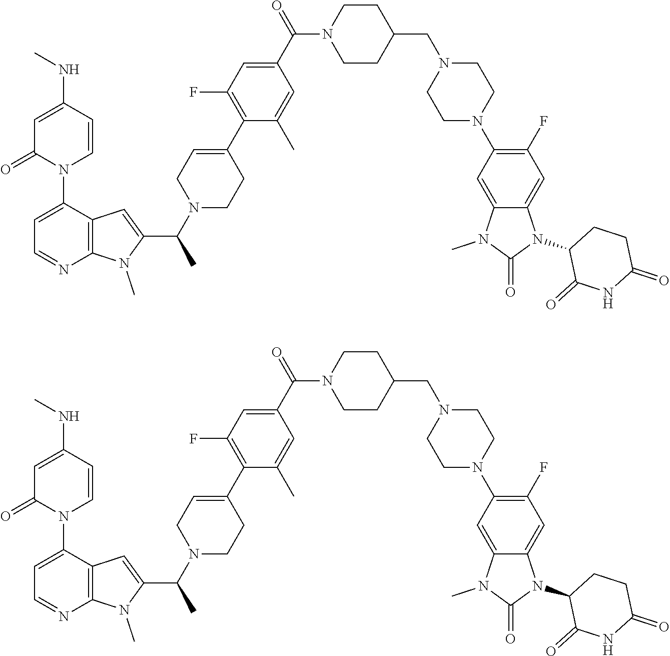 [SMILES: CNc1ccn(-c2ccnc3c2cc([C@H](C)N2CC=C(c4c(C)cc(C(=O)N5CCC(CN6CCN(c7cc8c(cc7F)n([C@@H]7CCC(=O)NC7=O)c(=O)n8C)CC6)CC5)cc4F)CC2)n3C)c(=O)c1 ; CNc1ccn(-c2ccnc3c2cc([C@H](C)N2CC=C(c4c(C)cc(C(=O)N5CCC(CN6CCN(c7cc8c(cc7F)n([C@H]7CCC(=O)NC7=O)c(=O)n8C)CC6)CC5)cc4F)CC2)n3C)c(=O)c1]

Example 3u308 and 3u307. (R)-3-(6-fluoro-5-(4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)-3-methyl-2-oxo-2,3-dihydro-1H-benzo[d]imidazol-1-yl)piperidine-2,6-dione and (R)-3-(6-fluoro-5-(4-((1-(3-fluoro-5-methyl-4-(1-((S)-1-(1-methyl-4-(4-(methylamino)-2-oxopyridin-1 (2H)-yl)-1H-pyrrolo[2,3-b]pyridin-2-yl)ethyl)-1,2,3,6-tetrahydropyridin-4-yl)benzoyl)piperidin-4-yl)methyl)piperazin-1-yl)-3-methyl-2-oxo-2,3-dihydro-1H-benzo[d]imidazol-1-yl)piperidine-2,6-dione

Example 3u310 (0.19 g) was resolved by HPLC using a Chiralpak IF PACKED 50×30 mm 5 μm column with an eluent of 35% ACN, 35% IPA and 30% DCM (48 mL/min) to give Example 3u308 (14 mg, first peak) and Example 3u307 (20 mg, second peak). The absolute configurations were not determined at the glutarimide chiral centers for these compounds.

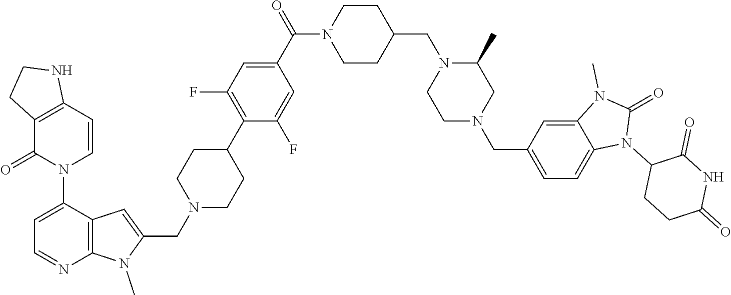 [SMILES: C[C@H]1CN(Cc2ccc3c(c2)n(C)c(=O)n3C2CCC(=O)NC2=O)CCN1CC1CCN(C(=O)c2cc(F)c(C3CCN(Cc4cc5c(-n6ccc7c(c6=O)CCN7)ccnc5n4C)CC3)c(F)c2)CC1]

### Example 3u311. 3-(5-(((S)-4-((1-(3,5-difluoro-4-(1-((1-methyl-4-(4-oxo-1,2,3,4-tetrahydro-5H-pyrrolo[3,2-c]pyridin-5-yl)-1H-pyrrolo[2,3-b]pyridin-2-yl)methyl)piperidin-4-yl)benzoyl)piperidin-4-yl)methyl)-3-methylpiperazin-1-yl)methyl)-3-methyl-2-oxo-2,3-dihydro-1H-benzo[d]imidazol-1-yl)piperidine-2,6-dione

Example 3u311 (28 mg, TFA salt) was prepared similarly to Example 3u163 from Ints. 2G57/3E68 (0.15 g/0.15 g).

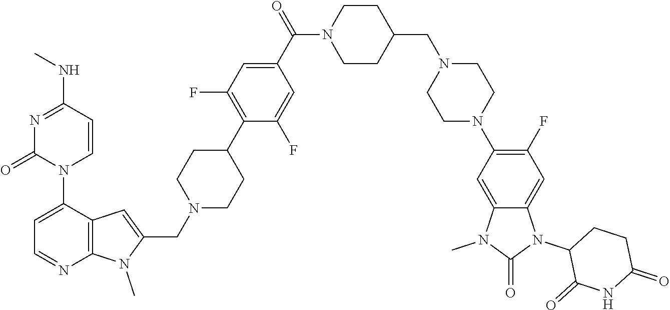 [SMILES: CNc1ccn(-c2ccnc3c2cc(CN2CCC(c4c(F)cc(C(=O)N5CCC(CN6CCN(c7cc8c(cc7F)n(C7CCC(=O)NC7=O)c(=O)n8C)CC6)CC5)cc4F)CC2)n3C)c(=O)n1]

### Example 3h43. 3-(5-(4-((1-(3,5-difluoro-4-(1-((1-methyl-4-(4-(methylamino)-2-oxopyrimidin-1 (2H)-yl)-1H-pyrrolo[2,3-b]pyridin-2-yl)methyl)piperidin-4-yl)benzoyl)piperidin-4-yl)methyl)piperazin-1-yl)-6-fluoro-3-methyl-2-oxo-2,3-dihydro-1H-benzo[d]imidazol-1-yl)piperidine-2,6-dione

Example 3h43 (24 mg) was prepared similarly to Example 2h2 from Ints. 3C10/2T37 (0.10 g/0.12 g).

### Characterization Data

| No. | Method | RT | Purity | MassObs | MassCalcd |
|---|---|---|---|---|---|
| 3u99 | 34 | 3.91 | 96.12 | 995.55 | 994.5 |
| 3u97 | 18 | 1.35 | 98.47 | 991.99 | 990.5 |
| 3u96 | 34 | 3.82 | 99.76 | 991.5 | 990.5 |
| 3u95 | 34 | 3.77 | 98.49 | 991.5 | 990.5 |
| 3u94 | 34 | 3.78 | 97.75 | 991.54 | 990.5 |
| 3u93 | 34 | 3.78 | 98.04 | 991.56 | 990.5 |
| 3u92 | 34 | 3.7 | 98.83 | 984.63 | 983.5 |
| 3u91 | 34 | 3.72 | 94.51 | 984.59 | 983.5 |
| 3u90 | 34 | 3.74 | 99.34 | 982.53 | 983.5 |
| 3u9 | 34 | 3.97 | 98.32 | 1001.64 | 1000.5 |
| 3u89 | 34 | 3.86 | 97.51 | 983.56 | 982.5 |
| 3u88 | 34 | 3.86 | 97 | 983.52 | 982.5 |
| 3u87 | 34 | 3.85 | 99.3 | 983.67 | 982.5 |
| 3u86 | 33 | 7.34 | 97.2 | 983.18 | 982.5 |
| 3u85 | 34 | 3.67 | 97.5 | 982.51 | 981.5 |
| 3u84 | 34 | 3.89 | 98.62 | 987.51 | 986.5 |
| 3u83 | 34 | 3.61 | 94.02 | 986.46 | 985.5 |
| 3u82 | 34 | 3.95 | 97.47 | 985.6 | 984.5 |
| 3u81 | 34 | 3.95 | 99.14 | 985.64 | 984.5 |
| 3u80 | 34 | 3.46 | 98.72 | 985.6 | 984.5 |
| 3u8 | 34 | 3.8 | 97.01 | 1000.48 | 999.5 |
| 3u79 | 34 | 3.85 | 96.76 | 987.61 | 986.5 |
| 3u78 | 34 | 3.85 | 96.08 | 987.61 | 986.5 |
| 3u77 | 34 | 3.86 | 98.92 | 987.57 | 986.5 |
| 3u76 | 10 | 9.12 | 95.25 | 982.53 | 981.5 |
| 3u75 | 10 | 9.13 | 95.04 | 982.62 | 981.5 |
| 3u74 | 34 | 3.64 | 97.69 | 982.66 | 981.5 |
| 3u73 | 34 | 3.64 | 98.35 | 982.58 | 981.5 |
| 3u72 | 34 | 3.65 | 98.87 | 982.58 | 981.5 |
| 3u71 | 34 | 3.65 | 99.57 | 982.58 | 981.5 |
| 3u70 | 34 | 3.69 | 95.75 | 982.55 | 981.5 |
| 3u7 | 34 | 3.7 | 99.72 | 947.63 | 946.5 |
| 3u69 | 31 | 1.36 | 97.38 | 981.66 | 980.5 |
| 3u68 | 34 | 3.76 | 95.02 | 981.59 | 980.5 |
| 3u67 | 34 | 3.77 | 94.57 | 981.63 | 980.5 |
| 3u66 | 34 | 3.75 | 97.07 | 981.61 | 980.5 |
| 3u65 | 34 | 3.94 | 96.61 | 981.59 | 980.5 |
| 3u64 | 34 | 3.95 | 98.36 | 981.59 | 980.5 |
| 3u63 | 34 | 3.97 | 95.57 | 981.54 | 980.5 |
| 3u62 | 34 | 3.68 | 95.09 | 976.63 | 975.5 |
| 3u61 | 34 | 3.69 | 95.02 | 976.63 | 975.5 |
| 3u60 | 34 | 3.68 | 99.29 | 976.61 | 975.5 |
| 3u6 | 34 | 3.71 | 99.81 | 947.63 | 946.5 |
| 3u59 | 34 | 3.78 | 97.86 | 975.58 | 974.5 |
| 3u58 | 34 | 3.77 | 97.74 | 975.54 | 974.5 |
| 3u57 | 34 | 3.79 | 96.94 | 975.54 | 974.5 |
| 3u56 | 34 | 3.6 | 99.24 | 974.53 | 973.5 |
| 3u55 | 34 | 3.6 | 95.13 | 974.53 | 973.5 |
| 3u54 | 34 | 3.64 | 98.59 | 974.6 | 973.5 |
| 3u53 | 34 | 3.62 | 97.88 | 974.53 | 973.5 |
| 3u52 | 34 | 3.62 | 98.11 | 974.53 | 973.5 |
| 3u51 | 34 | 3.61 | 97.38 | 974.55 | 973.5 |
| 3u50 | 34 | 3.91 | 97.56 | 989.59 | 988.5 |
| 3u5 | 34 | 3.71 | 98.46 | 947.59 | 946.5 |
| 3u49 | 34 | 3.9 | 97.77 | 989.63 | 988.5 |
| 3u48 | 34 | 3.93 | 99.26 | 989.59 | 988.5 |
| 3u47 | 34 | 3.76 | 96.79 | 969.6 | 968.5 |
| 3u46 | 34 | 3.76 | 98 | 969.64 | 968.5 |
| 3u45 | 34 | 3.76 | 98.88 | 969.6 | 968.5 |
| 3u44 | 34 | 3.71 | 99.52 | 973.52 | 972.5 |
| 3u43 | 34 | 3.7 | 97.37 | 973.52 | 972.5 |
| 3u42 | 34 | 3.69 | 97.84 | 973.59 | 972.5 |
| 3u41 | 10 | 8.98 | 91.55 | 966.78 | 965.5 |
| 3u40 | 10 | 8.99 | 94.29 | 964.6 | 965.5 |
| 3u4 | 34 | 4.02 | 98.92 | 1013.54 | 1012.5 |
| 3u39 | 34 | 3.55 | 91.08 | 964.56 | 965.5 |
| 3u38 | 34 | 3.82 | 98.25 | 965.59 | 964.5 |
| 3u37 | 34 | 3.82 | 99.34 | 965.55 | 964.5 |
| 3u36 | 34 | 3.83 | 97.35 | 965.61 | 964.5 |
| 3u35 | 41 | 0.811 | 100 | 964.4 | 965.5 |
| 3u34 | 34 | 3.63 | 98.06 | 966.62 | 965.5 |
| 3u33 | 34 | 3.63 | 98.45 | 966.62 | 965.5 |
| 3u32 | 34 | 3.67 | 96.73 | 966.62 | 965.5 |
| 3u311 | 34 | 3.61 | 98.7 | 970.54 | 969.5 |
| 3u310 | 34 | 3.71 | 95.74 | 956.54 | 955.5 |
| 3u31 | 34 | 3.52 | 98.69 | 944.57 | 943.5 |
| 3u309 | 19 | 1.64 | 96.08 | 1009.62 | 1008.5 |
| 3u308 | 34 | 3.7 | 95 | 956.52 | 955.5 |
| 3u307 | 34 | 3.7 | 96.07 | 956.52 | 955.5 |
| 3u306 | 34 | 3.98 | 97.01 | 987.51 | 986.5 |
| 3u305 | 34 | 3.93 | 95.42 | 985.49 | 984.5 |
| 3u304 | 31 | 1.4 | 98.14 | 951.54 | 950.5 |
| 3u303 | 31 | 1.43 | 98.8 | 969.56 | 968.5 |
| 3u302 | 19 | 4.45 | 98.74 | 967.73 | 966.5 |
| 3u301 | 32 | 1.75 | 99.48 | 959.56 | 958.4 |
| 3u300 | 30 | 4.24 | 97.32 | 971.58 | 970.4 |
| 3u30 | 34 | 3.52 | 98.68 | 944.57 | 943.5 |
| 3u3 | 34 | 3.57 | 85.19 | 956.45 | 955.5 |
| 3u299 | 30 | 4.12 | 95.42 | 938.6 | 937.5 |
| 3u298 | 34 | 3.71 | 95.01 | 956.52 | 955.5 |
| 3u297 | 38 | 2.79 | 98.15 | 956.62 | 955.5 |
| 3u296 | 38 | 2.81 | 96.26 | 956.75 | 955.5 |
| 3u295 | 30 | 1.45 | 95.83 | 981.62 | 980.5 |
| 3u294 | 34 | 3.62 | 98.45 | 938.54 | 937.5 |
| 3u293 | 34 | 3.6 | 98.16 | 938.54 | 937.5 |
| 3u292 | 34 | 3.82 | 98.23 | 967.56 | 966.5 |
| 3u291 | 34 | 3.83 | 98.23 | 967.52 | 966.5 |
| 3u290 | 34 | 3.82 | 98.54 | 971.51 | 970.4 |
| 3u29 | 34 | 3.48 | 99.04 | 944.61 | 943.5 |
| 3u289 | 34 | 3.77 | 97.74 | 959.54 | 958.4 |
| 3u288 | 34 | 3.77 | 96.7 | 959.5 | 958.4 |
| 3u287 | 34 | 3.7 | 98.45 | 964.58 | 963.5 |
| 3u286 | 34 | 3.91 | 95.12 | 969.58 | 968.5 |
| 3u285 | 34 | 3.91 | 95.06 | 969.53 | 968.5 |
| 3u284 | 34 | 4.07 | 95.18 | 966.55 | 965.5 |
| 3u283 | 34 | 3.69 | 98.07 | 982.51 | 981.5 |
| 3u282 | 34 | 3.98 | 95.21 | 966.51 | 965.5 |
| 3u281 | 34 | 3.81 | 95.98 | 951.6 | 950.5 |
| 3u280 | 34 | 3.81 | 97.68 | 951.56 | 950.5 |
| 3u28 | 34 | 3.56 | 98.22 | 962.54 | 961.5 |
| 3u279 | 34 | 3.7 | 98.21 | 964.54 | 963.5 |
| 3u278 | 34 | 3.69 | 98.24 | 964.54 | 963.5 |
| 3u277 | 41 | 0.897 | 100 | 974.4 | 975.5 |
| 3u276 | 34 | 3.82 | 95.44 | 982.55 | 981.5 |
| 3u275 | 41 | 0.806 | 100 | 976.6 | 975.5 |
| 3u274 | 34 | 3.77 | 99.67 | 966.47 | 965.4 |
| 3u273 | 34 | 3.8 | 96.92 | 974.49 | 973.5 |
| 3u272 | 34 | 3.79 | 99.69 | 956.52 | 955.5 |
| 3u271 | 34 | 3.91 | 96.11 | 956.52 | 955.5 |
| 3u270 | 34 | 3.66 | 98.68 | 948.45 | 947.4 |
| 3u27 | 34 | 3.57 | 97.3 | 962.54 | 961.5 |
| 3u269 | 34 | 3.88 | 97.64 | 990.45 | 989.4 |
| 3u268 | 34 | 3.69 | 99.07 | 975.46 | 974.5 |
| 3u267 | 34 | 3.58 | 98.79 | 925.42 | 924.5 |
| 3u265 | 34 | 3.62 | 97 | 920.42 | 919.4 |
| 3u264 | 34 | 3.42 | 98.67 | 958.47 | 957.4 |
| 3u263 | 30 | 1.57 | 98.6 | 945.57 | 944.4 |
| 3u262 | 30 | 1.66 | 99.78 | 904.53 | 903.4 |
| 3u261 | 16 | 1.74 | 99.66 | 967.69 | 966.4 |
| 3u260 | 41 | 0.917 | 97.4 | 963.4 | 961.5 |
| 3u26 | 34 | 3.55 | 99.01 | 960.48 | 961.5 |
| 3u259 | 41 | 0.919 | 97.83 | 1002.4 | 1000.5 |
| 3u258 | 34 | 3.9 | 97.73 | 995.59 | 994.5 |
| 3u257 | 41 | 0.884 | 91.01 | 1005.6 | 1006.5 |
| 3u256 | 34 | 3.58 | 95.74 | 957.52 | 956.5 |
| 3u255 | 41 | 0.944 | 95.36 | 971 | 972.5 |
| 3u254 | 41 | 0.903 | 97.93 | 1002.4 | 1000.5 |
| 3u253 | 34 | 3.82 | 95.92 | 986.5 | 985.5 |
| 3u252 | 41 | 0.93 | 100 | 963.2 | 964.5 |
| 3u251 | 34 | 3.83 | 98.75 | 963.49 | 962.4 |
| 3u250 | 19 | 4.53 | 93.08 | 971.61 | 970.5 |
| 3u25 | 10 | 8.93 | 94.23 | 964.6 | 963.5 |
| 3u249 | 10 | 9.96 | 99.65 | 961.56 | 960.5 |
| 3u248 | 34 | 3.83 | 97.68 | 947.53 | 946.5 |
| 3u247 | 34 | 3.8 | 98.38 | 945.47 | 944.4 |
| 3u246 | 34 | 3.62 | 96.46 | 994.48 | 993.4 |
| 3u245 | 34 | 3.93 | 97.38 | 969.49 | 968.4 |
| 3u244 | 34 | 3.82 | 96.66 | 951.48 | 950.4 |
| 3u243 | 34 | 3.96 | 96.38 | 978.52 | 977.5 |
| 3u242 | 34 | 3.98 | 96.64 | 978.52 | 977.5 |
| 3u241 | 34 | 3.99 | 97.4 | 978.48 | 977.5 |
| 3u240 | 34 | 3.76 | 98.37 | 1005.48 | 1004.5 |
| 3u24 | 10 | 8.92 | 93.42 | 964.6 | 963.5 |
| 3u239 | 34 | 3.84 | 98.19 | 990.49 | 989.5 |
| 3u238 | 41 | 0.861 | 98.81 | 964.4 | 965.5 |
| 3u237 | 34 | 3.73 | 98.43 | 979.55 | 978.4 |
| 3u236 | 34 | 4.14 | 95.07 | 988.54 | 987.5 |
| 3u235 | 34 | 4.2 | 95.18 | 990.55 | 989.5 |
| 3u234 | 34 | 4.21 | 96.67 | 990.6 | 989.5 |
| 3u233 | 34 | 4.23 | 98.22 | 990.55 | 989.5 |
| 3u232 | 34 | 4.07 | 97.84 | 972.5 | 971.5 |
| 3u231 | 34 | 3.62 | 94.04 | 960.44 | 959.5 |
| 3u230 | 34 | 3.99 | 95.82 | 958.51 | 957.5 |
| 3u23 | 10 | 8.92 | 92.35 | 964.6 | 963.5 |
| 3u229 | 34 | 3.97 | 96.65 | 958.47 | 957.5 |
| 3u228 | 34 | 3.99 | 98 | 958.47 | 957.5 |
| 3u227 | 34 | 3.95 | 97.68 | 959.48 | 958.5 |
| 3u226 | 34 | 3.86 | 97.68 | 959.48 | 958.5 |
| 3u225 | 34 | 3.89 | 95.05 | 959.48 | 958.5 |
| 3u224 | 34 | 3.74 | 98.16 | 969.68 | 968.5 |
| 3u223 | 34 | 3.73 | 97.58 | 969.56 | 968.5 |
| 3u222 | 34 | 3.74 | 98.39 | 969.56 | 968.5 |
| 3u221 | 34 | 3.83 | 97.82 | 987.57 | 986.5 |
| 3u220 | 34 | 3.83 | 97.43 | 987.61 | 986.5 |
| 3u22 | 10 | 8.89 | 92.46 | 962.58 | 963.5 |
| 3u219 | 34 | 3.83 | 98.29 | 987.57 | 986.5 |
| 3u218 | 34 | 3.67 | 98.52 | 984.59 | 983.5 |
| 3u217 | 34 | 3.7 | 98.38 | 984.59 | 983.5 |
| 3u216 | 34 | 3.7 | 97.96 | 984.55 | 983.5 |
| 3u215 | 34 | 3.74 | 99.57 | 1000.59 | 999.5 |
| 3u214 | 34 | 3.75 | 99.17 | 1000.55 | 999.5 |
| 3u213 | 34 | 3.72 | 99.69 | 1000.51 | 999.5 |
| 3u212 | 34 | 3.75 | 95.01 | 996.56 | 995.5 |
| 3u211 | 34 | 3.74 | 97.96 | 996.64 | 995.5 |
| 3u210 | 34 | 3.72 | 96.72 | 996.52 | 995.5 |
| 3u21 | 34 | 4.17 | 95.22 | 964.5 | 963.5 |
| 3u209 | 34 | 3.72 | 95.89 | 984.55 | 983.5 |
| 3u208 | 34 | 3.7 | 96.79 | 984.55 | 983.5 |
| 3u207 | 34 | 3.7 | 98.85 | 984.51 | 983.5 |
| 3u206 | 34 | 3.81 | 98.25 | 971.57 | 970.5 |
| 3u205 | 34 | 3.19 | 97.92 | 971.53 | 970.5 |
| 3u204 | 34 | 3.77 | 98.91 | 971.49 | 970.5 |
| 3u203 | 34 | 3.86 | 98.47 | 983.58 | 982.5 |
| 3u202 | 34 | 3.87 | 96.9 | 983.58 | 982.5 |
| 3u201 | 34 | 3.73 | 98.09 | 983.5 | 982.5 |
| 3u200 | 35 | 1.65 | 96.58 | 1013.55 | 1012.5 |
| 3u20 | 34 | 4.05 | 98.12 | 984.53 | 983.5 |
| 3u2 | 34 | 3.57 | 90.15 | 956.45 | 955.5 |
| 3u199 | 34 | 3.8 | 97.33 | 994.54 | 993.5 |
| 3u198 | 34 | 3.78 | 98.07 | 994.54 | 993.5 |
| 3u197 | 34 | 3.79 | 96.24 | 994.59 | 993.5 |
| 3u196 | 34 | 3.82 | 95.01 | 1010.55 | 1009.5 |
| 3u195 | 34 | 3.84 | 97.97 | 995.51 | 994.5 |
| 3u194 | 34 | 3.84 | 98.46 | 995.51 | 994.5 |
| 3u193 | 34 | 3.85 | 98.3 | 995.59 | 994.5 |
| 3u192 | 34 | 3.76 | 98.63 | 977.49 | 976.5 |
| 3u191 | 8 | 3.76 | 98.02 | 977.54 | 976.5 |
| 3u190 | 34 | 3.77 | 98.43 | 977.54 | 976.5 |
| 3u19 | 34 | 3.63 | 96.95 | 958.53 | 957.5 |
| 3u189 | 34 | 3.78 | 96.1 | 949.52 | 948.5 |
| 3u188 | 34 | 3.8 | 97.24 | 949.57 | 948.5 |
| 3u187 | 34 | 3.77 | 94.34 | 949.57 | 948.5 |
| 3u186 | 34 | 3.79 | 96.94 | 961.58 | 960.5 |
| 3u185 | 34 | 3.81 | 95.01 | 961.62 | 960.5 |
| 3u184 | 34 | 3.74 | 96.24 | 961.58 | 960.5 |
| 3u183 | 34 | 3.94 | 97.42 | 997.57 | 996.5 |
| 3u182 | 34 | 3.93 | 95.13 | 997.57 | 996.5 |
| 3u181 | 34 | 3.95 | 96.53 | 997.61 | 996.5 |
| 3u180 | 34 | 3.76 | 96.89 | 965.57 | 964.5 |
| 3u18 | 34 | 3.69 | 98.06 | 957.52 | 956.5 |
| 3u179 | 34 | 3.4 | 97.16 | 965.61 | 964.5 |
| 3u178 | 22 | 3.96 | 99.24 | 965.49 | 964.5 |
| 3u177 | 34 | 3.87 | 96.85 | 979.68 | 978.5 |
| 3u176 | 34 | 3.84 | 97.91 | 979.55 | 978.5 |
| 3u175 | 34 | 3.88 | 96.44 | 979.68 | 978.5 |
| 3u174 | 34 | 3.97 | 98..00 | 997.61 | 996.5 |
| 3u173 | 34 | 3.99 | 97.44 | 997.61 | 996.5 |
| 3u172 | 34 | 10.07 | 99.46 | 997.69 | 996.5 |
| 3u171 | 34 | 3.95 | 99.49 | 983.63 | 982.5 |
| 3u170 | 34 | 3.95 | 97.98 | 983.67 | 982.5 |
| 3u17 | 34 | 3.69 | 98.01 | 957.52 | 956.5 |
| 3u169 | 34 | 3.85 | 98.48 | 983.96 | 982.5 |
| 3u168 | 34 | 3.81 | 99.22 | 1010.63 | 1009.5 |
| 3u167 | 34 | 3.81 | 99.16 | 1010.63 | 1009.5 |
| 3u166 | 34 | 3.87 | 96.85 | 1010.55 | 1009.5 |
| 3u165 | 34 | 3.92 | 97.86 | 991.56 | 990.5 |
| 3u164 | 34 | 3.93 | 98.9 | 995.61 | 994.5 |
| 3u163 | 34 | 3.43 | 97.51 | 938.56 | 937.5 |
| 3u162 | 34 | 3.43 | 95.31 | 938.52 | 937.5 |
| 3u161 | 8 | 3.44 | 92.24 | 938.52 | 937.5 |
| 3u160 | 34 | 3.44 | 95.38 | 940.54 | 939.5 |
| 3u16 | 34 | 3.68 | 98.28 | 957.52 | 956.5 |
| 3u159 | 34 | 3.52 | 99.6 | 940.58 | 939.5 |
| 3u158 | 34 | 3.46 | 99.22 | 940.54 | 939.5 |
| 3u157 | 34 | 3.52 | 98.57 | 952.59 | 951.5 |
| 3u156 | 34 | 3.53 | 97.71 | 952.55 | 951.5 |
| 3u155 | 10 | 8.92 | 99.09 | 952.55 | 951.5 |
| 3u154 | 34 | 3.84 | 96.46 | 999.6 | 998.5 |
| 3u153 | 34 | 3.85 | 97.69 | 999.6 | 998.5 |
| 3u152 | 34 | 3.85 | 99.39 | 999.58 | 998.5 |
| 3u151 | 34 | 3.69 | 95.9 | 941.53 | 940.5 |
| 3u150 | 34 | 3.7 | 97.94 | 941.48 | 940.5 |
| 3u15 | 34 | 3.63 | 96.25 | 970.56 | 969.5 |
| 3u149 | 32 | 1.72 | 98.98 | 941.51 | 940.5 |
| 3u148 | 34 | 3.71 | 98.24 | 1008.61 | 1007.5 |
| 3u147 | 34 | 3.71 | 96.44 | 1008.7 | 1007.5 |
| 3u146 | 10 | 9.37 | 90.7 | 1008.66 | 1007.5 |
| 3u145 | 33 | 7.2 | 41.92 | 1004.8 | 1003.5 |
| 3u144 | 10 | 9.5 | 93.17 | 1002.61 | 1003.5 |
| 3u143 | 10 | 9.49 | 95.16 | 1002.61 | 1003.5 |
| 3u142 | 34 | 3.62 | 95.06 | 990.6 | 989.5 |
| 3u140 | 10 | 9.16 | 91.49 | 990.64 | 989.5 |
| 3u14 | 34 | 3.63 | 98.41 | 970.61 | 969.5 |
| 3u139 | 23 | 4.56 | 52.05 | 978.74 | 977.5 |
| 3u138 | 34 | 3.65 | 89.72 | 978.8 | 977.5 |
| 3u137 | 10 | 9.2 | 92.12 | 978.71 | 977.5 |
| 3u136 | 34 | 3.97 | 95.33 | 985.49 | 984.5 |
| 3u135 | 34 | 3.94 | 98.03 | 1022.61 | 1021.5 |
| 3u134 | 34 | 3.82 | 98.02 | 1022.57 | 1021.5 |
| 3u133 | 34 | 3.87 | 93.7 | 1022.56 | 1021.5 |
| 3u132 | 34 | 3.87 | 95.47 | 1022.56 | 1021.5 |
| 3u131 | 8 | 3.86 | 95.53 | 1022.56 | 1021.5 |
| 3u130 | 34 | 3.86 | 97.17 | 1014.53 | 1013.5 |
| 3u13 | 34 | 3.64 | 98.3 | 970.56 | 969.5 |
| 3u129 | 34 | 3.85 | 97.67 | 1014.53 | 1013.5 |
| 3u128 | 34 | 3.84 | 97.85 | 1014.62 | 1013.5 |
| 3u127 | 34 | 3.92 | 96 | 1011.48 | 1010.5 |
| 3u126 | 34 | 3.98 | 95.37 | 981.46 | 980.4 |
| 3u125 | 34 | 3.67 | 99.01 | 978.81 | 977.5 |
| 3u124 | 10 | 9.18 | 98.64 | 978.63 | 977.5 |
| 3u123 | 10 | 9.18 | 98.37 | 978.63 | 977.5 |
| 3u122 | 34 | 3.61 | 98.86 | 978.59 | 977.5 |
| 3u121 | 34 | 3.89 | 95.36 | 999.56 | 998.5 |
| 3u120 | 34 | 3.85 | 96.27 | 1010.6 | 1009.5 |
| 3u12 | 34 | 3.6 | 99.22 | 970.61 | 969.5 |
| 3u119 | 34 | 3.88 | 97.62 | 1010.64 | 1009.5 |
| 3u118 | 34 | 3.88 | 98.7 | 1010.67 | 1009.5 |
| 3u117 | 34 | 3.85 | 96.02 | 1007.58 | 1006.5 |
| 3u116 | 34 | 3.93 | 95.84 | 1007.54 | 1006.5 |
| 3u115 | 34 | 3.98 | 98.74 | 1005.52 | 1004.5 |
| 3u114 | 34 | 3.96 | 98.3 | 995.53 | 994.5 |
| 3u113 | 10 | 9.5 | 98.62 | 992.65 | 991.5 |
| 3u112 | 34 | 3.74 | 98.49 | 992.61 | 991.5 |
| 3u111 | 34 | 3.74 | 99.35 | 992.74 | 991.5 |
| 3u110 | 41 | 0.969 | 97.71 | 992 | 990.5 |
| 3u11 | 34 | 3.6 | 97.38 | 970.56 | 969.5 |
| 3u109 | 34 | 3.78 | 97.48 | 996.54 | 995.5 |
| 3u108 | 10 | 9.36 | 95.01 | 996.7 | 995.5 |
| 3u107 | 10 | 9.36 | 97.06 | 996.7 | 995.5 |
| 3u106 | 28 | 6.86 | 95.11 | 996.84 | 995.5 |
| 3u105 | 10 | 9.4 | 99.04 | 996.64 | 995.5 |
| 3u104 | 10 | 9.4 | 99.1 | 996.64 | 995.5 |
| 3u103 | 34 | 3.72 | 99.26 | 996.77 | 995.5 |
| 3u102 | 34 | 3.79 | 98.34 | 996.58 | 995.5 |
| 3u101 | 34 | 3.92 | 98.05 | 995.57 | 994.5 |
| 3u100 | 34 | 3.91 | 96.44 | 995.55 | 994.5 |
| 3u10 | 34 | 3.57 | 99.48 | 970.56 | 969.5 |
| 3u1 | 34 | 3.55 | 97.19 | 956.58 | 955.5 |
| 3h12 | 34 | 3.62 | 98.68 | 970.58 | 969.5 |
| 3h10 | 34 | 3.74 | 98.42 | 988.56 | 987.5 |
| 3e44 | 3 | 3.72 | 99.1 | 948.7 | 947.5 |
| 3e39 | 8 | 3.62 | 99.6 | 936.7 | 935.5 |
| 3h43 | 34 | 3.5 | 96.6 | 948.4 | 949.5 |

Method refers to one of LC/MS methods 1-41
RT = retention time in min
Purity is based on LC/MS (UV) or by ¹H NMR (if indicated with *)

### Example 3—Pharmacology Data for the Compounds of the Disclosure

| | | STAT6 | |
|---|---|---|---|
| | Eotaxin-3 EC₅₀ | degradation | |
| No. | (nM) | DC₅₀ (nM) | STAT6 degradation Eₘₐₓ (%) |
| 3e39 | 0.5 | 0.5 | |
| 3e44 | 0.3 | 0.7 | |
| 3h10 | 4.8 | 3.2 | |
| 3h12 | 1.3 | 3.2 | |
| 3u1 | 1.0 | 0.9 | |
| 3u10 | | 0.6 | |
| 3u100 | 3.1 | 1.7 | |
| 3u101 | 2.4 | 2.3 | |
| 3u102 | 1.9 | 3.4 | |
| 3u103 | | 2.5 | |
| 3u104 | | 0.9 | |
| 3u105 | | 1.4 | |
| 3u106 | 0.8 | 2.3 | |
| 3u107 | 2.8 | 2.5 | |
| 3u108 | 4.1 | 3.4 | |
| 3u109 | 2.8 | 1.4 | |
| 3u11 | 0.2 | 0.5 | |
| 3u110 | | 2.5 | |
| 3u111 | 0.3 | 0.4 | |
| 3u112 | | 0.5 | |
| 3u113 | | 0.6 | |
| 3u114 | | 9.9 | |
| 3u115 | 9.1 | 8.3 | |
| 3u116 | 6.0 | 8.7 | |
| 3u117 | 1210 | | |
| 3u118 | 2.2 | 1.2 | |
| 3u119 | 2.2 | 2.1 | |
| 3u12 | 0.5 | 0.6 | |
| 3u120 | 3.7 | 1.0 | |
| 3u121 | 2.0 | 2.5 | |
| 3u122 (3u124)* | 0.6 | 1.2 | |
| 3u123 (3u124)* | | 0.9 | |
| 3u125 | 0.6 | 0.8 | |
| 3u126 | 0.2 | 0.4 | |
| 3u127 | 14.8 | 5.4 | |
| 3u128 | 1.6 | 1.1 | |
| 3u129 | 1.4 | 1.0 | |
| 3u13 | 1.0 | 0.7 | |
| 3u130 | 1.8 | 1.3 | |
| 3u131 | | 0.8 | |
| 3u132 | | 0.5 | |
| 3u133 | | 0.8 | |
| 3u134 | | | 20-30 |
| 3u135 | 185 | | |
| 3u136 | 16.4 | | |
| 3u137 | 0.4 | 0.2 | |
| 3u139 (3u138)* | | 0.2 | |
| 3u14 | 1.2 | 0.6 | |
| 3u140 | 1.0 | 1.4 | |
| 3u142 | | 2.4 | |
| 3u143 | | 0.7 | |
| 3u144 | | 0.4 | |
| 3u145 | | 2.0 | |
| 3u146 | 1.4 | 1.7 | |
| 3u147 | | 1.6 | |
| 3u148 | | 2.7 | |
| 3u149 | 27.6 | 33.8 | |
| 3u15 | 2.2 | 0.8 | |
| 3u150 | 39.2 | | |
| 3u151 | | | 30-40 |
| 3u152 | 13.2 | 6.6 | |
| 3u153 | 8.7 | 4.1 | |
| 3u154 | 10.1 | 6.4 | |
| 3u155 | 0.5 | 0.3 | |
| 3u156 | | 0.6 | |
| 3u157 | 0.4 | 0.3 | |
| 3u158 | | 24.1 | |
| 3u159 | 52.6 | 12.8 | |
| 3u16 | 12.1 | 11.2 | |
| 3u160 | 27.5 | 10.9 | |
| 3u161 | 1.7 | 3.1 | |
| 3u162 | 2.3 | 3.3 | |
| 3u163 | | 4.4 | |
| 3u164 | 2.0 | 1.0 | |
| 3u165 (3u97)* | | 1.4 | |
| 3u166 | 1.3 | 1.1 | |
| 3u167 | | 0.8 | |
| 3u168 | | 2.1 | |
| 3u169 | 14.6 | 4.1 | |
| 3u17 | 6.3 | 6.0 | |
| 3u170 | 7.5 | 2.5 | |
| 3u171 | 15.4 | 3.5 | |
| 3u172 | 13.2 | 2.9 | |
| 3u173 | 7.8 | 3.1 | |
| 3u174 | 11 | 3.8 | |
| 3u175 | 3.8 | 1.3 | |
| 3u176 | 9.8 | 2.3 | |
| 3u177 | 3.1 | 1.8 | |
| 3u178 | 7.7 | 5.6 | |
| 3u179 | 12.4 | 6.1 | |
| 3u18 | 18.6 | 8.4 | |
| 3u180 | 17.3 | 8.8 | |
| 3u181 | 16.3 | 3.4 | |
| 3u182 | 7.0 | 5.9 | |
| 3u183 | 8.0 | 5.9 | |
| 3u184 | 7.0 | 2.4 | |
| 3u185 | 2.3 | 2.8 | |
| 3u186 | 4.6 | 3.5 | |
| 3u187 | 4.2 | 3.1 | |
| 3u188 | 3.7 | 3.4 | |
| 3u189 | 7.7 | 4.7 | |
| 3u19 | 0.3 | 0.7 | |
| 3u190 | 0.9 | 0.5 | |
| 3u191 | 0.5 | 0.4 | |
| 3u192 | 0.9 | 0.6 | |
| 3u193 | 2.4 | 1.9 | |
| 3u194 | 0.9 | 1.7 | |
| 3u195 | 2.2 | 1.6 | |
| 3u196 | 0.5 | 0.3 | |
| 3u197 | 2.7 | 1.2 | |
| 3u198 | 1.6 | 1.2 | |
| 3u199 | 2.7 | 1.1 | |
| 3u2 | | 4.3 | |
| 3u20 | 11.7 | 4.7 | |
| 3u200 | 9.9 | 2.0 | |
| 3u201 | 3.8 | 1.3 | |
| 3u202 | 2.6 | 1.9 | |
| 3u203 | 5.2 | 3.4 | |
| 3u204 | 4.6 | 2.5 | |
| 3u205 | 5.9 | 3.0 | |
| 3u206 | 10.9 | 0.4 | |
| 3u207 | 0.2 | 0.3 | |
| 3u208 | 0.8 | 0.6 | |
| 3u209 | 0.7 | 7.3 | |
| 3u21 | | 0.4 | |
| 3u210 | 1.1 | 0.5 | |
| 3u211 | 0.5 | 0.5 | |
| 3u212 | 0.8 | 7.5 | |
| 3u213 | 13.2 | 8.6 | |
| 3u214 | 4.5 | 13.7 | |
| 3u215 | 5.6 | 3.5 | |
| 3u216 | 1.5 | 3.5 | |
| 3u217 | 0.6 | 1.8 | |
| 3u218 | 0.8 | 2.6 | |
| 3u219 | 2.2 | 1.8 | |
| 3u22 | 0.8 | 0.7 | |
| 3u220 | 2.4 | 1.3 | |
| 3u221 | 4.5 | 1.9 | |
| 3u222 | 1.1 | 1.5 | |
| 3u223 | 1.3 | 0.9 | |
| 3u224 | 1.5 | 1.3 | |
| 3u225 | 0.3 | 0.1 | |
| 3u226 | 0.2 | 0.05 | |
| 3u227 | 0.3 | 0.04 | |
| 3u228 | 1.2 | 0.2 | |
| 3u229 | 1.6 | 0.3 | |
| 3u23 | | 1.3 | |
| 3u230 | 2.0 | 0.4 | |
| 3u231 | 0.8 | 0.1 | |
| 3u232 | 0.8 | 0.1 | |
| 3u233 | 1.3 | 0.2 | |
| 3u234 | | 0.1 | |
| 3u235 | 1.4 | 0.1 | |
| 3u236 | 0.9 | 0.1 | |
| 3u237 | 1.7 | 0.1 | |
| 3u238 | 3.2 | 1.4 | |
| 3u239 | 1.2 | 0.1 | |
| 3u24 | | 1.2 | |
| 3u240 | 1.3 | 1.1 | |
| 3u241 | 0.2 | 0.1 | |
| 3u242 | 0.2 | 0.2 | |
| 3u243 | 0.4 | 0.3 | |
| 3u244 | 0.3 | 0.1 | |
| 3u245 | 0.1 | 0.1 | |
| 3u246 | 1.1 | 2.7 | |
| 3u247 | 0.3 | 0.04 | |
| 3u248 | 0.3 | 0.2 | |
| 3u249 | 1.4 | 0.6 | |
| 3u25 | 1.0 | 1.3 | |
| 3u250 | 0.4 | 0.1 | |
| 3u251 | 0.9 | 0.2 | |
| 3u252 | | 0.4 | |
| 3u253 | 8.1 | | |
| 3u254 | 0.2 | 0.4 | |
| 3u255 | | 13.7 | |
| 3u256 | 0.8 | 2.5 | |
| 3u257 | 24.2 | | |
| 3u258 | 7.0 | 3.1 | |
| 3u259 | | 0.5 | |
| 3u26 | 3.4 | 2.9 | |
| 3u260 | 11.2 | | |
| 3u261 | | | <20 |
| 3u262 | | | 30-40 |
| 3u263 | | | 70-80 |
| 3u264 | | 5.9 | |
| 3u265 | | | <20 |
| 3u267 | | | 50-60 |
| 3u268 | 5.6 | | |
| 3u269 | 96.3 | | |
| 3u27 | 5.7 | 2.0 | |
| 3u270 | 26.8 | | |
| 3u271 | 3.3 | | |
| 3u272 | | | <20 |
| 3u273 | 31.8 | | |
| 3u274 | | | <20 |
| 3u275 | | 57.4 | |
| 3u276 | | 4.3 | |
| 3u277 | | 0.4 | |
| 3u278 | 3.1 | 3.1 | |
| 3u279 | 1.7 | 1.4 | |
| 3u28 | 24.3 | 2.9 | |
| 3u280 | 12.8 | | |
| 3u281 | 4.2 | 4.0 | |
| 3u282 | 6.9 | | |
| 3u283 | 126 | | |
| 3u284 | 24 | 22.6 | |
| 3u285 | 101 | 7.8 | |
| 3u286 | 25.2 | 12.5 | |
| 3u287 | 2.9 | 2.7 | |
| 3u288 | 88.3 | | |
| 3u289 | 18.9 | | |
| 3u29 | 0.9 | 1.7 | |
| 3u291 | 21.7 | 5.7 | |
| 3u292 | 6.9 | 5.1 | |
| 3u293 | 0.8 | 1.0 | |
| 3u294 | 0.3 | 0.8 | |
| 3u295 | 18.9 | 4.5 | |
| 3u296 | 5.8 | 2.7 | |
| 3u297 | 3.2 | 2.2 | |
| 3u298 | 2.6 | 1.8 | |
| 3u299 | 0.5 | 0.9 | |
| 3u3 | | 1.8 | |
| 3u30 | 1.6 | 2.2 | |
| 3u300 (3u290)* | 24.1 | 13 | |
| 3u301 | 90.5 | | 20-30 |
| 3u302 | 29.2 | 4.9 | |
| 3u303 | 37.1 | 21.7 | |
| 3u304 | 10.8 | 6.5 | |
| 3u305 | 24.9 | | |
| 3u306 | 194 | | |
| 3u307 | 1.1 | 1.4 | |
| 3u308 | 1.3 | 0.7 | |
| 3u309 | 4.8 | 2.2 | |
| 3u31 | 2.0 | 2.4 | |
| 3u310 | 0.4 | 0.7 | |
| 3u311 | 0.4 | 0.5 | |
| 3u32 | 0.3 | 0.2 | |
| 3u33 | 0.3 | 0.2 | |
| 3u34 | | 0.3 | |
| 3u35 | 5.5 | 9.6 | |
| 3u36 | 3.3 | 1.4 | |
| 3u37 | 1.7 | 0.5 | |
| 3u38 | 1.5 | 1.3 | |
| 3u39 | 2.3 | 4.3 | |
| 3u4 | | 4.0 | |
| 3u40 | | 2.3 | |
| 3u41 | | 6.0 | |
| 3u42 | 13.8 | 13.2 | |
| 3u43 | 9.1 | 8.5 | |
| 3u44 | 10.1 | 10.2 | |
| 3u45 | 4.5 | 3.9 | |
| 3u46 | 10.1 | 5.8 | |
| 3u47 | 8.0 | 0.8 | |
| 3u48 | 7.7 | 11.2 | |
| 3u49 | 12.9 | 1.7 | |
| 3u5 | 0.8 | 1.0 | |
| 3u50 | 30.2 | 0.9 | |
| 3u51 | 1.9 | 1.8 | |
| 3u52 | 1.1 | 4.3 | |
| 3u53 | 1.1 | 8.7 | |
| 3u54 | 2.7 | 12 | |
| 3u55 | 3.1 | 7.6 | |
| 3u56 | 3.5 | 8.7 | |
| 3u57 | 25.4 | 12 | |
| 3u58 | 12.6 | 7.6 | |
| 3u59 | 25.8 | 11.4 | |
| 3u6 | 19.3 | 0.7 | |
| 3u60 | 1.6 | 0.7 | |
| 3u61 | 1.2 | 1.9 | |
| 3u62 | 1.2 | 1.2 | |
| 3u63 | 67.3 | 12.9 | |
| 3u64 | 50.3 | 7.3 | |
| 3u65 | 93.5 | | |
| 3u66 | 4.3 | 1.6 | |
| 3u67 | 5.8 | 1.4 | |
| 3u68 | 7.8 | 1.0 | |
| 3u69 | 2.4 | 1.5 | |
| 3u7 | | 0.6 | |
| 3u70 | | 5.8 | |
| 3u71 | 1.8 | 5.3 | |
| 3u72 | 1.9 | 4.5 | |
| 3u73 | 2.4 | 6.8 | |
| 3u74 | 1.1 | 0.6 | |
| 3u75 | 1.0 | 0.6 | |
| 3u76 | 1.3 | 3.2 | |
| 3u77 | 24.1 | 16.1 | |
| 3u78 | 45.9 | 9.8 | |
| 3u79 | 107 | 15.4 | |
| 3u8 | 5.9 | | |
| 3u80 | 5.0 | 0.8 | |
| 3u81 | | 0.8 | |
| 3u82 | 1.1 | 1.1 | |
| 3u83 | 8.0 | | |
| 3u84 | | 26 | |
| 3u85 | 5.2 | 3.1 | |
| 3u86 | 5.1 | 3.4 | |
| 3u87 | 15.2 | 3.5 | |
| 3u88 | 12.9 | 3.3 | |
| 3u89 | 43.7 | 4.1 | |
| 3u9 | 4.9 | 3.8 | |
| 3u90 | 0.9 | 0.3 | |
| 3u91 | 1.4 | 0.6 | |
| 3u92 | 1.8 | 0.4 | |
| 3u93 | 16.6 | 26.9 | |
| 3u94 | 23.8 | 10.2 | |
| 3u95 | 12.4 | 18.6 | |
| 3u96 | 1.0 | 4.1 | |
| 3u99 | 1.7 | 1.1 | |
| 3h43 | 7.4 | | |

Eotaxin-3 EC₅₀ values provided for test compounds with >80% Eₘₐₓ
STAT6 degradation DC₅₀ values provided for test compounds with >80% Eₘₐₓ Data is provided as geometric mean values across multiple assay runs and compound batches to the extent possible
*The activity of diastereomeric mixtures and their corresponding pure diastereomers is illustrated for several cases in this table such as 3u118/3u119/3u120 and 3u90/3u91/3u92 and also shown graphically for 3u118/3u119/3u120 in the method descriptions below.

### Stat6 Degrader Assay

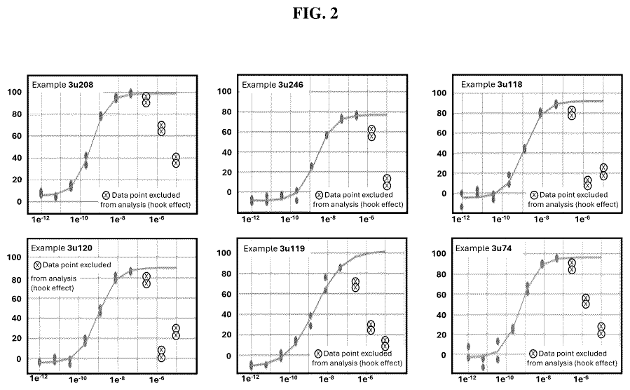

This was a HiBit based assay. A HeLa cell line stably transfected with a small protein, LgBit (Large Bit) was obtained from Promega. These cells express endogenous STAT6. A small 11 amino acid gene sequence (HiBiT) was inserted in the STAT6 gene using CRISPR technology. Once STAT6 protein is expressed in the cells, the HiBiT sequence will bind the LgBit in the cells resulting in a functional NanoBiT protein, giving a luminescent tag to STAT6, see FIG. 2.

Single cell clones were prepared and the clone 4.14 was selected for the assay based on that clone showing high luminescence, high STAT6 phosphorylation upon stimulation with IL-4, and inhibition of luminescence when cells were treated with STAT6 siRNA.

HeLa LgBiT clone 4.14 cells were suspended in DMEM with 10% FCS, 1% Pen/Strep, Na-pyrovate and Hygromycin B, at a concentration of 1.25*105 cells pr mL. 5000 cells were added pr well of a 384 well plate and 20 nL of compound was added by Echo® liquid handler. NN6394A (terfenadine, a toxic compound) was added to control wells at 50 μM concentration to establish the assay window. In these wells the cells are dead which equals 100% degradation of STAT6.

After 4-24 h of incubation at 37° C. under 5% CO₂/95% air, 5 μL substrate (Nano-GLO®) was added to the wells and the plates were centrifuged and incubated for 0.5 h.

The luminescence in the wells as a measure of non-degraded STAT6 was measured in a plate reader (BMG Pherastar FSX). The measured DC₅o (in nM) was determined and reported in FIG. 3.

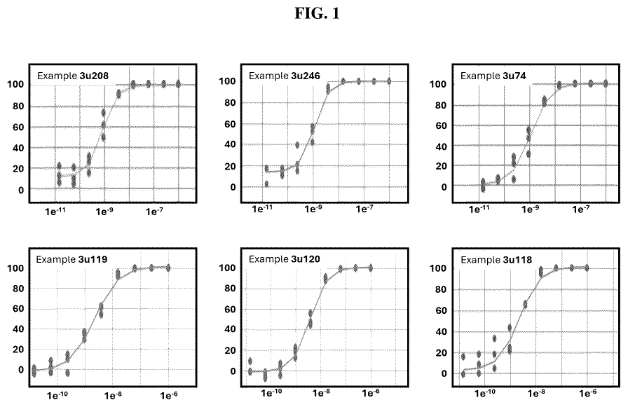

The dose-response curves for Examples 3u208, 3u246, 3u118, 3u119, 3u120, and 3u74 are shown in FIG. 1 (where percent degradation is plotted vs. test compound concentration in M on a log-scale).

### Human Whole Blood Eotaxin-3 Assay

This was a human whole blood assay using recombinant human Interleukin 4 to stimulate the blood and measuring the ability of the test compounds to inhibit eotaxin-3 release. The biological activities of the test compounds have been tested in human whole blood stimulated with IL-4 measuring Eotaxin-3 release. The test compounds were added to 384 well clear flat-bottom plate in a 4-fold serial dilution in triplicates using a liquid handler. Human whole blood stabilized in lithium-heparin tubes was added in a volume of 65 μL per well, resulting in a final DMSO concentration of 0.5%. The plate is incubated for 2 h at 37° C. under 5% CO₂/95% air. Subsequently, 5 μL of recombinant human IL-4 (R&D Systems, cat #204-ILB) was added to the wells to a final concentration of 400 μM. The plate was incubated for 48 h at 37° C. under 5% CO₂/95% air. The plate was spun down for 10 minutes at 500×g and 20 μL supernatant was harvested and transferred to a 384 well v-bottom plate using a liquid handler and stored at −20° C. until further analysis. 10 μL supernatant was used to measure the level of Eotaxin-3 (CCL26) using Human Eotaxin-3 MSD kit from Mesocsale (Cat #K251UEK-4). The assay was performed according to the manufacture instructions.

The data from the human whole blood eotaxin-3 assay is shown in FIG. 2 for Examples 3u208, 3u246, 3u118, 3u119, 3u120, and 3u74 (where the effect in % is plotted against the test concentration in M on a log scale).

Unless otherwise defined, all technical and scientific terms used herein have the same meaning as commonly understood by one of ordinary skill in the art to which this disclosure belongs.

The disclosure illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by certain embodiments and optional features, modification, improvement and variation of the disclosure embodied therein herein disclosed may be resorted to by those skilled in the art, and that such modifications, improvements and variations are considered to be within the scope of this disclosure. The materials, methods, and examples provided here are representative of certain embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

### EMBODIMENTS

1. A compound having the formula (I″)

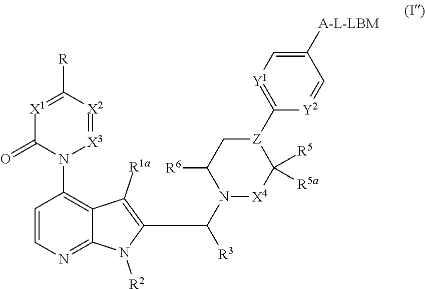

wherein:
X¹ is selected from N and CR¹¹; X² is selected from N and CR¹²; and X³ is selected from N and CR¹³, provided that only one of X¹, X² and X³ may be N;
X⁴ is CR⁴ or CO;
Y¹ is selected from N and CR⁷;
Y² is selected from N and CR⁸;
Z is selected from N and CR¹⁰;
R is NHR⁰;
R⁰ is selected from hydrogen, C₁₋₄alkyl, C₃₋₄ cycloalkyl, C₃₋₄ cycloalkyl-C₁₋₄alkyl, phenyl-C₁₋₄alkyl, halo-C₁₋₄alkyl, —(CH₂)ₙ—O—C₁₋₄alkyl, —(CH₂)ₙ—CO—R′, and —(CH₂)ₙ—CO₂R, wherein said phenyl is optionally substituted one or more times with a substituents independently selected from halogen, C₁₋₄alkyl, halo-C₁₋₄alkyl, C₃₋₄cycloalkyl, hydroxy, C₁₋₄alkoxy, cyano, —NR′R″, —CONR′R″ and —CO₂R′, and wherein R′ and R″ are each independently selected from hydrogen and C₁₋₄alkyl, and n is 0, 1 or 2;
R^{1a} is hydrogen;
R² is selected from hydrogen, C₁₋₅alkyl and deuterated C₁₋₄alkyl; wherein said C₁₋₄alkyl may optionally be substituted one or more times with halogen;
R³ is selected from hydrogen, C₁₋₄alkyl and deuterated C₁₋₄alkyl;
R⁴ and R⁶ are independently selected from hydrogen and C₁₋₄alkyl wherein said C₁₋₄alkyl may optionally be substituted with one or more halogens;
R⁵ is selected from hydrogen, halogen, C₁₋₄alkyl and C₁₋₄alkoxy, wherein said C₁₋₄alkyl and C₁₋₄alkoxy may optionally be substituted with one or more halogens;
R^{5a} is hydrogen;
or R⁵ and R¹⁰ together form a bond;
or R⁵ and R^{5a} are both fluoro;
R⁷ and R⁸ is independently selected from hydrogen, halogen, C₁₋₄alkyl, C₃₋₄ cycloalkyl, and C₁₋₄alkoxy, wherein said C₁₋₄alkyl and C₁₋₄alkoxy may optionally be substituted with one or more halogen;
A is CO;
L is selected from

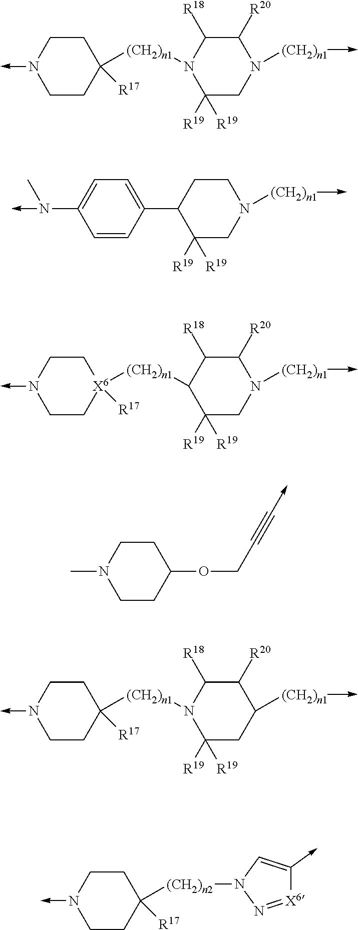

wherein n1 is selected from 0 and 1;
n2 is selected from 1 and 2;
R¹⁷ is selected from hydrogen and fluoro;
R¹⁸ is selected from hydrogen, fluoro, C₁₋₄alkyl wherein said C₁₋₄alkyl may optionally be substituted one or more times with fluoro or C₁₋₄alkoxy;
R¹⁹ is independently selected from hydrogen, C₁₋₄alkyl and fluoro;
R²⁰ is selected from hydrogen and C₁₋₄alkyl;
X^{6′} is C or N;
LBM is

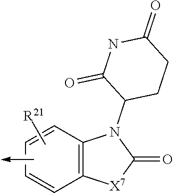

wherein X⁷ is O, NR²², CR²³R²³;
R²¹ is selected from hydrogen, fluoro and chloro;
R²² is selected from hydrogen, C₁₋₄alkyl, deuterated C₁₋₄alkyl and C₃₋₄ cycloalkyl, wherein said C₁₋₄alkyl may optionally be substituted one or more times with fluoro, hydroxy, C₁₋₄alkoxy and C₃₋₄ cycloalkyl;
R²³ is independently selected from hydrogen and C₁₋₄alkyl;
R¹⁰ is selected from hydrogen and fluoro; or R⁵ and R¹⁰ together form a bond;
R¹¹ is selected from hydrogen, halogen, C₁₋₄alkyl, C₃₋₄ cycloalkyl, C₁₋₄alkoxy, hydroxy, cyano, —NR′R″, —CONR′R″, —CO₂R′, —CO—(CH₂)ₘOH, wherein said C₁₋₄alkyl is optionally substituted one or more times with a substituent independently selected from halogen, hydroxy and C₁₋₄alkoxy, and wherein m is 1, 2 or 3 and wherein R′ and R″ are each independently selected from hydrogen and C₁₋₄alkyl; or
R¹¹ and R⁰ may together form a 5-6 membered heteroaromatic ring containing one to two heteroatoms selected from N, wherein said 5-6 membered heteroaromatic ring containing one to two heteroatoms selected from N is optionally be substituted one or more times with a substituent independently selected from halogen, C₁₋₄alkyl, halo-C₁₋₄alkyl, C₃₋₄ cycloalkyl, hydroxy, C₁₋₄alkoxy, cyano, —NR′R″, —CONR′R″, and —CO₂R′, wherein said C₁₋₄alkyl may optionally be substituted one or more times with a substituent independently selected from halogen and C₁₋₄alkoxy, and wherein R′ and R″ are each independently selected from hydrogen and C₁₋₄alkyl;
R¹² and R¹³ are independently selected from hydrogen, halogen, C₁₋₄alkyl, C₃₋₄ cycloalkyl, C₁₋₄alkoxy, hydroxy, cyano, —NR′R″, —CONR′R″, —CO₂R′, —CO—CO₂R′, and —CO—(CH₂)ₘOH, wherein said C₁₋₄alkyl is optionally substituted one or more times with a substituent independently selected from halogen, hydroxy and C₁₋₄alkoxy, and wherein m is 1, 2 or 3 and wherein R′ and R″ are each independently selected from hydrogen and C₁;
R¹⁴ is selected from hydrogen and C₁₋₄alkyl;
R¹⁵ is independently selected from C₁₋₄alkyl and C₃₋₄cycloalkyl;
or pharmaceutically acceptable salts thereof.

Embodiment 2. A compound according to embodiment 1 having the formula (II) or (II′)

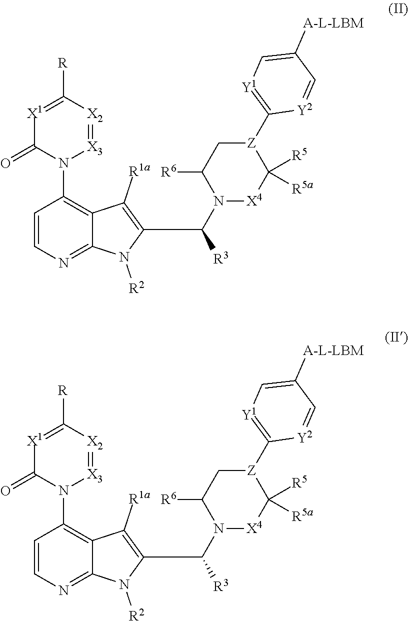

wherein X¹, X², X³, X⁴, Y¹, Y², Z, R, R^{1a}, R², R⁵, R^{5a}, R⁶, a, L and LBM are as defined in embodiment 1 and R³ is C₁₋₄alkyl; or pharmaceutically acceptable salts thereof.

Embodiment 3. The compound according to embodiment 1 having the formula (III)

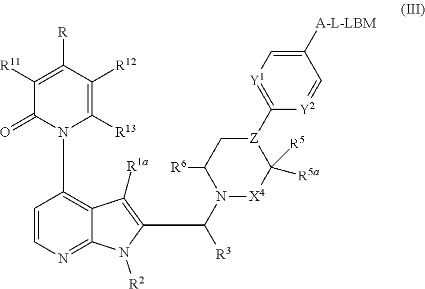

wherein Y¹, Y², X⁴, Z, R, R^{1a}, R², R³, R⁵, R^{5a}, R⁶, R⁹, R¹¹, R¹², R¹³, A, L and LBM are as defined in Embodiment 1 or pharmaceutically acceptable salts thereof.

Embodiment 4. The compound according to embodiment 3 having the formula (IV) or (IV′)

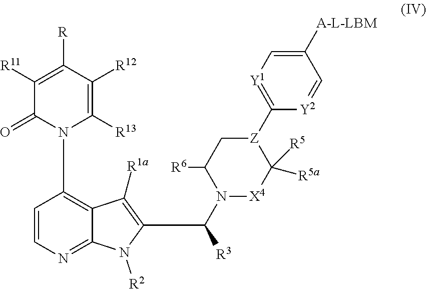

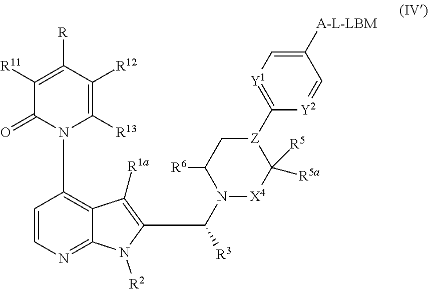

wherein Y¹, Y², X⁴, Z, R, R¹¹, R¹², R¹³, R^{1a}, R², R⁵, R^{5a}, R⁶, R⁹, R¹¹, R¹², R¹³, A, L and LBM are as defined in embodiment 3 and R³ is C₁₋₄alkyl; or pharmaceutically acceptable salts thereof.

Embodiment 5. The compound according to embodiment 1 having the formula (VII)

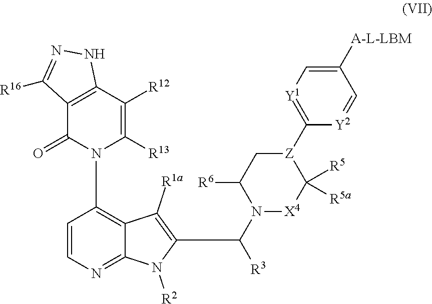

wherein Z, X⁴, Y¹, Y², R^{1a}, R², R³, R⁵, R^{5a}, R⁶, R⁹ R¹², R¹³, A, L and LBM are as defined in embodiment 1 and R¹⁶ is selected from hydrogen, C₁₋₄alkyl and trifluoromethyl.

Embodiment 6. The compound according to embodiment 5 having the formula (VIII) or (VIII′)

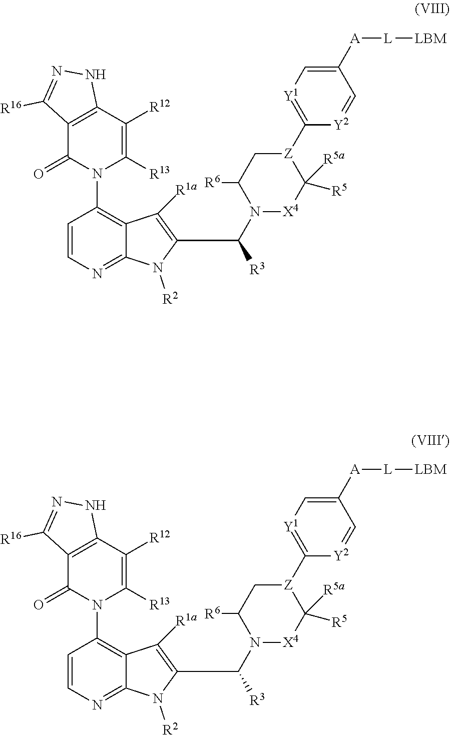

wherein Z, X⁴, Y¹, Y², R^{1a}, R², R³, R⁵, R^{5a}, R⁶, R⁹ R¹², R¹³, A, L and LBM are as defined in embodiment 1 and R¹⁶ is selected from hydrogen, C₁₋₄alkyl and trifluromethyl.

Embodiment 7. The compound according to any one of embodiments 1-2, wherein X¹ is N, X² is CR¹² and X³ is CR¹³.

Embodiment 8. The compound according to any one of embodiments 1-2, wherein X¹ is CR¹¹ and X² is N, and X³ is CR¹³.

Embodiment 9. The compound according to any one of embodiments 1-2, wherein X¹ is CR¹¹ and X² is CR¹² and X³ is N.

Embodiment 10. The compound according to any one of embodiments 1-9, wherein X⁴ is CR⁴ and R⁴ is as defined in claim 1.

Embodiment 11. The compound according to any one of embodiments 1-10, wherein Z is N.

Embodiment 12. The compound according to any one of embodiments 1-10, wherein Z is CR¹⁰ and R⁵ and R¹⁰ together form a bond.

Embodiment 13. The compound according to any one of embodiments 1-10, wherein Z is CR¹⁰ and R¹⁰ is hydrogen.

Embodiment 14. The compound according to any one of claims 1-13, wherein Y¹ is N and Y² is N.

Embodiment 15. The compound according to any one of claims 1-13, wherein Y¹ is N and Y² is CR⁸.

Embodiment 16. The compound according to any one of claims 1-13, wherein Y¹ is CR⁷ and Y² is CRB.

Embodiment 17. The compound according to any one of embodiment 16, wherein
(a) both of R⁷ and R⁸ is C₁₋₄alkyl;
(b) both of R⁷ and R⁸ is halogen;
(c) one of R⁷ and R⁸ is C₁₋₄alkyl and the other is halogen;
(d) one of R⁷ and R⁸ is C₁₋₄alkyl and the other is hydrogen; or
(e) one of R⁷ and R⁸ is halogen and the other is hydrogen.

Embodiment 18. The compound according to embodiment 17, wherein R⁷ is halogen and R⁸ is methyl.

Embodiment 19. The compound according to any one of embodiments 18 or 19, wherein the halogen is fluoro.

Embodiment 20. The compound according to any one of embodiments 1-4 and 7-19, wherein R is selected from —NH₂ and NHCH₃.

Embodiment 21. The compound according to any one of embodiments 1-20, wherein R¹² and R¹³ is hydrogen.

Embodiment 22. The compound according to any one of embodiments 1-4 and 7-19, wherein R¹¹ is C₁₋₄alkyl, and R¹² and R¹³ is hydrogen.

Embodiment 23. A compound according to any one of embodiments 1-22 wherein R² is methyl.

Embodiment 24. A compound according to any one of embodiments 1-23 wherein R³ is methyl. 20 Embodiment 25. A compound according to any one of embodiments 1-24 wherein R⁵, is hydrogen.

Embodiment 26. The compound according to any of embodiments 1-25, wherein -A-L-LBM is selected from

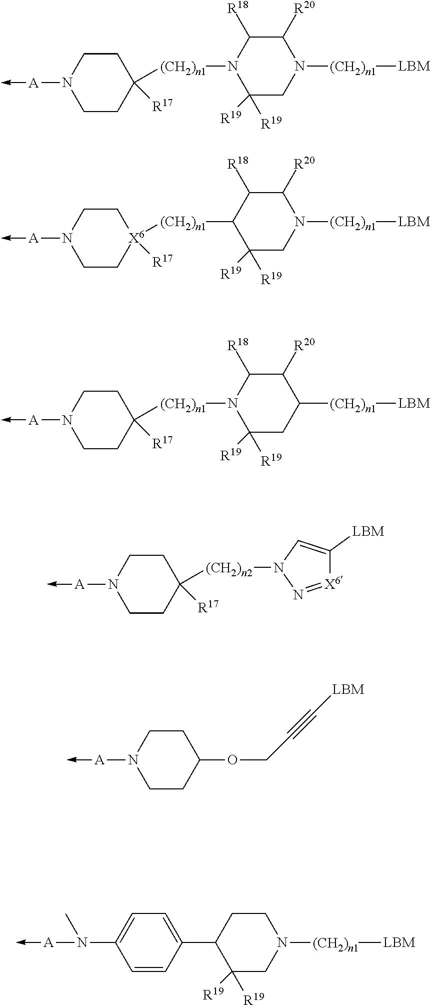

wherein A is CO;
n1 is selected from 0 and 1;
n2 is selected from 1 and 2;
R¹⁷ is selected from hydrogen and fluoro;
R¹⁸ is selected from hydrogen, fluoro, C₁₋₄alkyl wherein said C₁₋₄alkyl may optionally be substituted one or more times with fluoro or C₁₋₄alkoxy;
R¹⁹ is independently selected from hydrogen, C₁₋₄alkyl and fluoro;
R²⁰ is selected from hydrogen and C₁₋₄alkyl;
X^{6′} is C or N;
LBM is

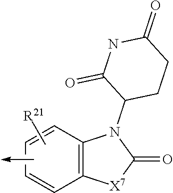

wherein X⁷ is O, NR²², CR²³R²³;
R²¹ is selected from hydrogen, fluoro and chloro;
R²² is selected from hydrogen, C₁₋₄alkyl, deuterated C₁₋₄alkyl and C₃₋₄ cycloalkyl, wherein said C₁₋₄alkyl may optionally be substituted one or more times with fluoro, hydroxy, C₁₋₄alkoxy and C₃_4 cycloalkyl; and
R²³ is independently selected from hydrogen and C₁₋₄alkyl.

Embodiment 27. The compound according to embodiment 26, wherein A-L-LBM is

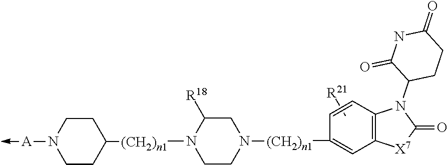

wherein A is CO, n1 is 1 and R¹⁸ and R²¹ is as defined in claim 1.

Embodiment 28. The compound according to embodiment 26, wherein A-L-LBM is

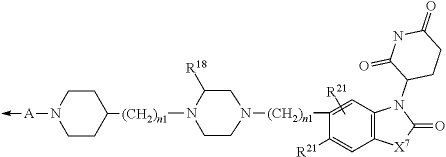

wherein A is CO, n is 1 and R¹⁸ is hydrogen or C₁₋₄alkyl and R²¹ is hydrogen and fluoro.

Embodiment 29. The compound according to embodiment 1, which is selected from 3-(3-methyl-5-(((S)-3-methyl-4-((1-(4-(1-((S)-1-(1-methyl-4-(4-(methylamino)-2-oxopyridin-1 (2H)-yl)-1H-pyrrolo[2,3-b]pyridin-2-yl)ethyl)piperidin-4-yl)benzoyl)piperidin-4-yl)methyl)piperazin-1-yl)methyl)-2-oxo-2,3-dihydro-1H-benzo[d]imidazol-1-yl)piperidine-2,6-dione and 3-(3-methyl-5-(((S)-3-methyl-4-((1-(4-(1-((S)-1-(1-methyl-4-(4-oxo-1,2,3,4-tetrahydro-5H-pyrrolo[3,2-c]pyridin-5-yl)-1H-pyrrolo[2,3-b]pyridin-2-yl)ethyl)piperidin-4-yl)benzoyl)piperidin-4-yl)methyl)piperazin-1-yl)methyl)-2-oxo-2,3-dihydro-1H-benzo[d]imidazol-1-yl)piperidine-2,6-dione or pharmaceutically acceptable salts thereof.

Embodiment 30. A compound according to any one of embodiments 1-29 for use in therapy.

Embodiment 31. A compound according to embodiment 30 for use in the treatment of a disease, disorder or condition, which disease, disorder or condition is responsive of modulation of STAT6.

Embodiment 32. A compound according to embodiment 30 for use in the treatment of autoimmune diseases.

Embodiment 33. A compound according to embodiment 30 for use in the treatment of autoimmune diseases, characterized by Th2-mediated inflammation such as atopic dermatitis, asthma, chronic rhinosinusitis with nasal polyposis, urticaria, rhinitis, eosinophilic esophagitis, food allergy, diffuse cutaneous systemic sclerosis, alopecia areata and/or COPD (chronic obstructive pulmonary disease) and different cancers such as lymphomas, triple negative breast cancer and solid fibrous cancers either as a stand-alone treatment or in combination with other anticancer drugs such as check point inhibitors.

Embodiment 34. A pharmaceutical composition comprising a compound according to any one of Embodiments 1-29 together with a pharmaceutically acceptable vehicle or excipient or pharmaceutically acceptable carrier(s).

1. A compound having the formula (I) 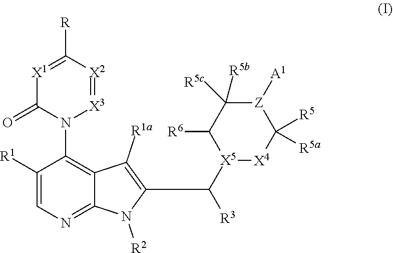
wherein:
A¹ is selected from 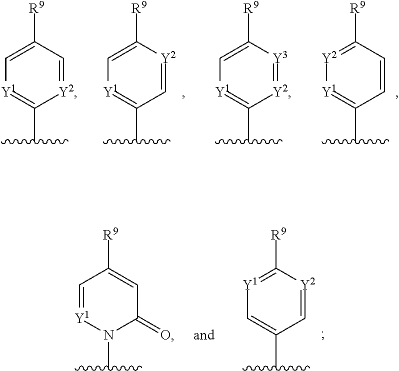
R⁹ is -A-L-LBM;
X¹ is selected from N and CR¹¹; X² is selected from N and CR¹²; and X³ is selected from N and CR¹³, provided that only one of X¹, X² and X³ may be N;
X⁴ is selected from CR⁴R⁴ and CO;
X⁵ is selected from N and oxidized N;
Y¹ is selected from N and CR⁷;
Y² is selected from N and CR⁸;
Y³ is selected from N and CR¹⁷;
Z is selected from N and CR¹⁰;
R is NHR⁰;
R⁰ is selected from hydrogen, C₁₋₄alkyl, C₃₋₄ cycloalkyl, C₃₋₄ cycloalkyl-C₁₋₄alkyl, phenyl-C₁₋₄alkyl, halo-C₁₋₄alkyl, —(CH₂)ₙ—O—C₁₋₄alkyl, —(CH₂)ₙ—CO—R′, and —(CH₂)ₙ—CO₂R′, wherein said phenyl is optionally substituted one or more times with a substituent independently selected from halogen, C₁₋₄alkyl, halo-C₁₋₄alkyl, C₃₋₄ cycloalkyl, hydroxy, C₁₋₄alkoxy, cyano, —NR′R″, —CONR′R″, and —CO₂R′, and wherein R′ and R″ are each independently selected from hydrogen and C₁₋₄alkyl, and n is 0, 1 or 2;
R¹ and R^{1a} are independently selected from hydrogen, fluoro, and C₁₋₄alkyl;
R² is selected from hydrogen, C₁₋₅alkyl, —SO₂—C₁₋₄ alkyl and deuterated C₁₋₄alkyl, wherein said C₁₋₅alkyl may optionally be substituted one or more times with halogen;
R³ is selected from hydrogen, C₁₋₄alkyl, and deuterated C₁₋₄alkyl;
each of R⁴ and R⁶ is independently selected from hydrogen, C₁₋₄alkyl, and C₁₋₄ alkoxy, said C₁₋₄alkyl and C₁₋₄alkoxy may optionally be substituted with one or more halogen;
R⁵ is selected from hydrogen, halogen, C₁₋₄alkyl and C₁₋₄alkoxy, wherein said C₁₋₄alkyl and C₁₋₄alkoxy may optionally be substituted with one or more halogen;
R¹⁰ is selected from hydrogen and fluoro;
or R⁵ and R¹⁰ together form a bond between the two carbons to which they are attached;
R^{5a} is hydrogen;
or R⁵ and R^{5a} are both fluoro or R⁵ and R^{5a} together with the atom attached thereto form a CO group;
R^{5b} and R^{5c} are each independently selected from hydrogen and fluoro;
R⁷, R⁸, and R¹⁷ are each independently selected from hydrogen, halogen, cyano, C₁₋₄alkyl and C₁₋₄alkoxy, and C₃₋₄cycloalkyl, wherein said C₁₋₄alkyl and C₁₋₄alkoxy may optionally be substituted with one or more halogen;
R¹¹, R¹², and R¹³ are each independently selected from hydrogen, halogen, C₁₋₄alkyl, C₃₋₄ cycloalkyl, C₁₋₄alkoxy, hydroxy, cyano, —NR′R″, —CONR′R″, —CO₂R′, and —CO—(CH₂)ₘOH, wherein said C₁₋₄alkyl is optionally substituted one or more times with a substituent independently selected from halogen, hydroxy and C₁₋₄alkoxy, and wherein m is 1, 2 or 3 and wherein R′ and R″ are each independently selected from hydrogen and C₁₋₄alkyl; or
R¹¹ and R⁰ together with the atoms attached thereto form a 5-6 membered heteroaromatic or heterocyclic ring containing one or two N atoms, wherein said 5-6 membered heteroaromatic ring or heterocyclic ring containing one or two N atoms is optionally substituted one or more times with a substituent independently selected from halogen, C₁₋₄alkyl, halo-C₁₋₄alkyl, C₃₋₄ cycloalkyl, hydroxy, C₁₋₄alkoxy, cyano, —COCF₃, —NR′R″, —CONR′R″, and —CO₂R′, wherein said C₁₋₄alkyl may optionally be substituted one or more times with a substituent independently selected from halogen and C₁₋₄alkoxy, and wherein R′ and R″ are each independently selected from hydrogen and C₁₋₄alkyl; and
R¹² and R¹³ are each independently selected from hydrogen, halogen, C₁₋₄alkyl, C₃₋₄ cycloalkyl, C₁₋₄alkoxy, hydroxy, cyano, —NR′R″, —CONR′R″, —CO₂R′, —CO—CO₂R′ and —CO—(CH₂)ₘOH, wherein said C₁₋₄alkyl is optionally substituted one or more times with a substituent independently selected from halogen, hydroxy and C₁₋₄alkoxy, and wherein m is 1, 2 or 3 and wherein R′ and R″ are each independently selected from hydrogen and C₁₋₄alkyl;
A is CO;
L is selected from 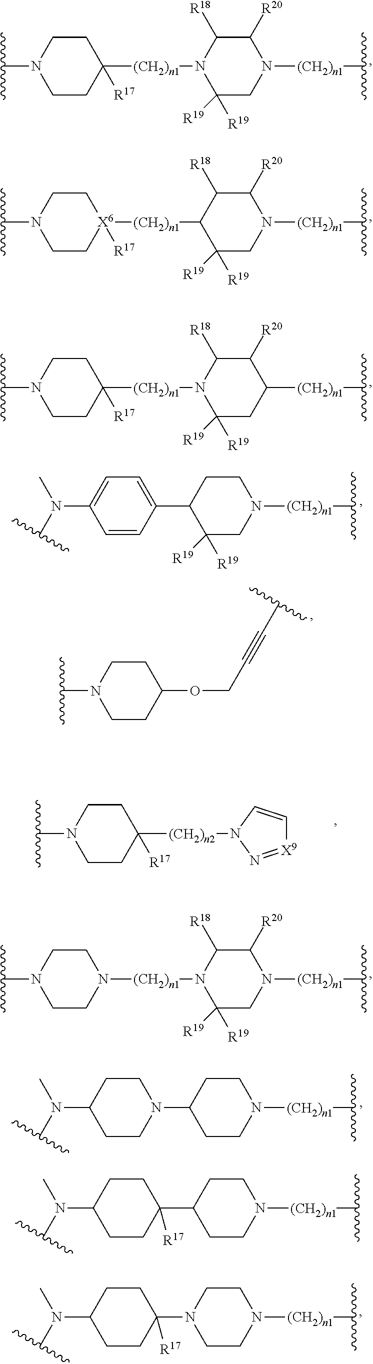 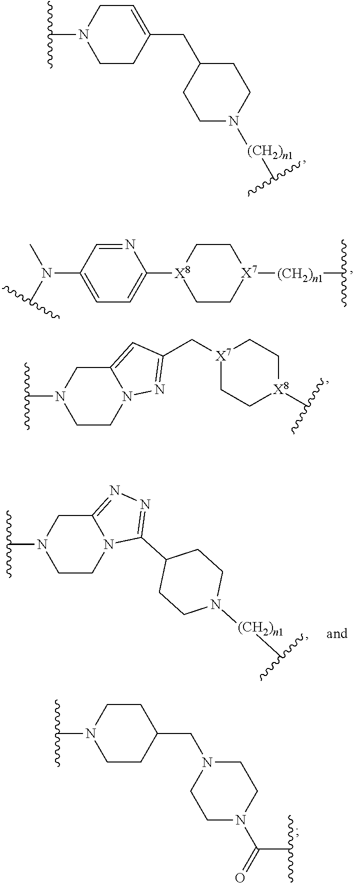
wherein n1 is selected from 0 and 1;
n2 is selected from 1 and 2;
R¹⁷ is selected from hydrogen and fluoro;
R¹⁸ is selected from hydrogen, fluoro, C₁₋₄alkyl wherein said C₁₋₄alkyl may optionally be substituted one or more times with fluoro or C₁₋₄alkoxy;
R¹⁹ is independently selected from hydrogen, C₁₋₄alkyl and fluoro;
R²⁰ is selected from hydrogen and C₁₋₄alkyl;
X⁶ is C;
X⁷ is CR²⁴ or N;
R²⁴ is selected from hydrogen and hydroxy;
X⁸ and X⁹ are independently CH or N; and
LBM is 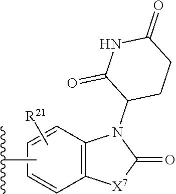
wherein X⁷ is O, NR²², or CR²³R²³;
R²¹ is selected from hydrogen, fluoro, and chloro;
R²² is selected from hydrogen, C₁₋₄alkyl, deuterated C₁₋₄alkyl, and C₃₋₄ cycloalkyl, wherein said C₁₋₄alkyl may optionally be substituted one or more times with fluoro, hydroxy, C₁₋₄alkoxy, or C₃₋₄ cycloalkyl;
each R²³ is independently selected from hydrogen, halogen, and C₁₋₄alkyl; or
both R²³ together with the carbon they are attached to form a C₃₋₆ cycloalkyl or 3-6 membered heterocyclic ring, wherein the heterocyclic ring contains one to two heteroatoms selected from O;
or a pharmaceutically acceptable salt or stereoisomer thereof.

2. The compound according to claim 1, having the formula (I′) 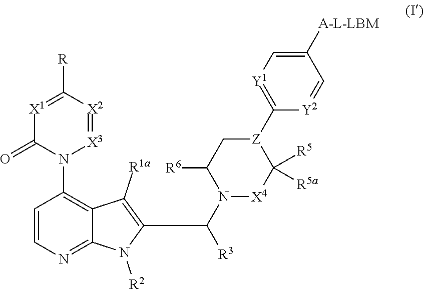
wherein:
X¹ is selected from N and CR¹¹; X² is selected from N and CR¹²; and X³ is selected from N and CR¹³, provided that only one of X¹, X² and X³ may be N;
X⁴ is CR⁴R⁴ or CO;
Y¹ is selected from N and CR⁷;
Y² is selected from N and CR⁸;
Z is selected from N and CR¹⁰;
R is NHR⁰;
R⁰ is selected from hydrogen, C₁₋₄alkyl, C₃₋₄ cycloalkyl, C₃₋₄ cycloalkyl-C₁₋₄alkyl, phenyl-C₁₋₄alkyl, halo-C₁₋₄alkyl, —(CH₂)ₙ—O—C₁₋₄alkyl, —(CH₂)ₙ—CO—R′, and —(CH₂)ₙ—CO₂R′, wherein said phenyl is optionally substituted one or more times with a substituent independently selected from halogen, C₁₋₄alkyl, halo-C₁₋₄alkyl, C₃₋₄ cycloalkyl, hydroxy, C₁₋₄alkoxy, cyano, —NR′R″, —CONR′R″, and —CO₂R′, and wherein R′ and R″ are each independently selected from hydrogen and C₁₋₄alkyl, and n is 0, 1, or 2;
R^{1a} is hydrogen;
R² is selected from hydrogen, C₁₋₄alkyl, and deuterated C₁₋₄alkyl; wherein said C₁₋₄alkyl may optionally be substituted one or more times with halogen;
R³ is selected from hydrogen, C₁₋₄alkyl, and deuterated C₁₋₄alkyl;
R⁴ and R⁶ are independently selected from hydrogen and C₁₋₄alkyl, wherein said C₁₋₄alkyl may optionally be substituted with one or more halogens;
R⁵ is selected from hydrogen, halogen, C₁₋₄alkyl, and C₁₋₄alkoxy, wherein said C₁₋₄alkyl and C₁₋₄alkoxy may optionally be substituted with one or more halogen;
R¹⁰ is selected from hydrogen and fluoro;
or R⁵ and R¹⁰ together form a bond between the two carbons to which they are attached;
R^{5a} is hydrogen; or R⁵ and R^{5a} are both fluoro;
R⁷ and R⁸ are independently selected from hydrogen, halogen, C₁₋₄alkyl, C₃₋₄ cycloalkyl, and C₁₋₄alkoxy, wherein said C₁₋₄alkyl and C₁₋₄alkoxy may independently optionally be substituted with one or more halogen;
R¹¹ is selected from hydrogen, halogen, C₁₋₄alkyl, C₃₋₄ cycloalkyl, C₁₋₄alkoxy, hydroxy, cyano, —NR′R″, —CONR′R″, —CO₂R′, —CO—(CH₂)ₘOH, wherein said C₁₋₄alkyl is optionally substituted one or more times with a substituent independently selected from halogen, hydroxy and C₁₋₄alkoxy, and wherein m is 1, 2 or 3 and wherein R′ and R″ are each independently selected from hydrogen and C₁₋₄alkyl; or
R¹¹ and R⁰ together with the atoms attached thereto may form a 5-6 membered heterocyclic or heteroaromatic ring containing one to two heteroatoms selected from N, wherein said 5-6 membered heterocyclic or heteroaromatic ring containing one to two heteroatoms selected from N is optionally substituted one or more times with a substituent independently selected from halogen, C₁₋₄alkyl, halo-C₁₋₄alkyl, C₃₋₄ cycloalkyl, hydroxy, C₁₋₄alkoxy, cyano, —NR′R″, —CONR′R″ and —CO₂R′, wherein said C₁₋₄alkyl may optionally be substituted one or more times with a substituent independently selected from halogen and C₁₋₄alkoxy, and wherein R′ and R″ are each independently selected from hydrogen and C₁₋₄alkyl;
R¹² and R¹³ are independently selected from hydrogen, halogen, C₁₋₄alkyl, C₃₋₄ cycloalkyl, C₁₋₄alkoxy, hydroxy, cyano, —NR′R″, —CONR′R″, —CO₂R′, —CO—CO₂R′ and —CO—(CH₂)ₘOH, wherein said C₁₋₄alkyl is optionally substituted one or more times with a substituent independently selected from halogen, hydroxy, and C₁₋₄alkoxy, and wherein m is 1, 2 or 3 and wherein R′ and R″ are each independently selected from hydrogen and C₁₋₄alkyl;
A is CO;
L is selected from 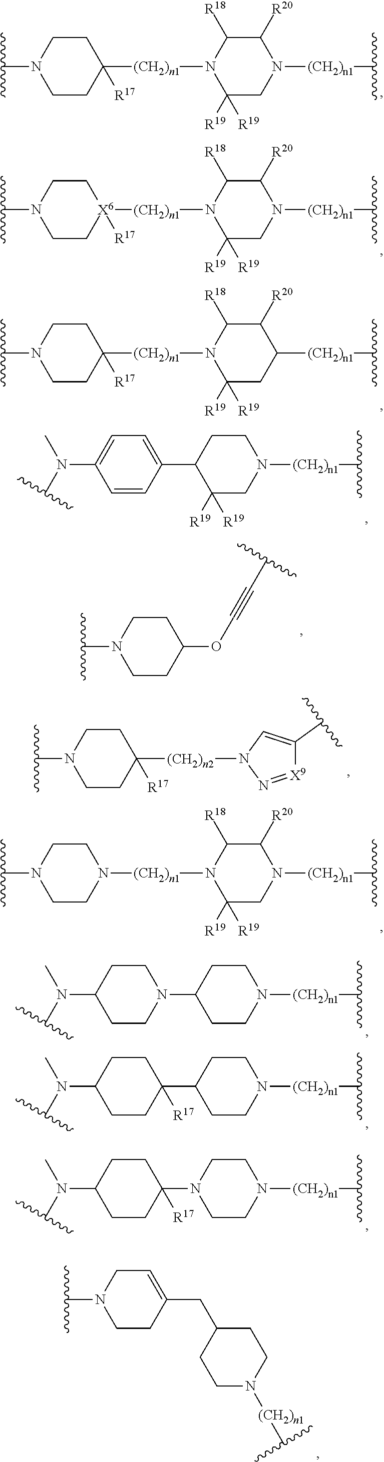 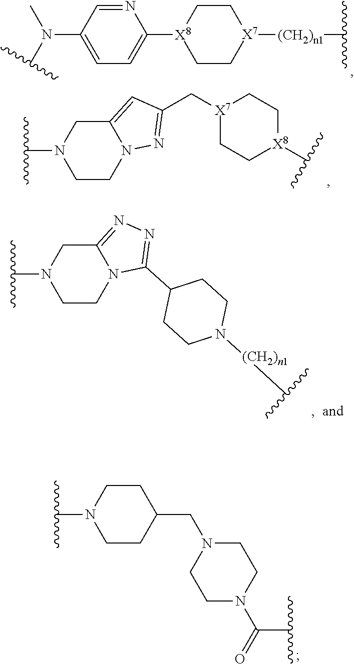
wherein n1 is selected from 0 and 1;
n2 is selected from 1 and 2;
R¹⁷ is selected from hydrogen and fluoro;
R¹⁸ is selected from hydrogen, fluoro, C₁₋₄alkyl wherein said C₁₋₄alkyl may optionally be
substituted one or more times with fluoro or C₁₋₄alkoxy;
R¹⁹ is independently selected from hydrogen, C₁₋₄alkyl and fluoro;
R²⁰ is selected from hydrogen and C₁₋₄alkyl;
X⁶ is C;
X⁷ is CR²⁴ or N;
R²⁴ is selected from hydrogen and hydroxy;
X⁸ and X⁹ are independently CH or N; and
LBM is 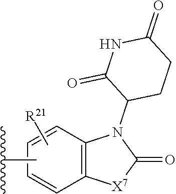
wherein X⁷ is O, NR²², or CR²³R²³;
R²¹ is selected from hydrogen, fluoro, and chloro;
R²² is selected from hydrogen, C₁₋₄alkyl, deuterated C₁₋₄alkyl, and C₃₋₄ cycloalkyl, wherein said C₁₋₄alkyl may optionally be substituted one or more times with fluoro, hydroxy, C₁₋₄alkoxy, or C₃₋₄ cycloalkyl;
each R²³ is independently selected from hydrogen, halogen, and C₁₋₄alkyl; or
both R²³ together with the carbon they are attached to form a C₃₋₆ cycloalkyl or 3-6 membered heterocyclic ring, wherein the heterocyclic ring contains one to two heteroatoms selected from O;
or a pharmaceutically acceptable salt or stereoisomer thereof.

3. A compound according to claim 2, having the formula (II) or (II′) 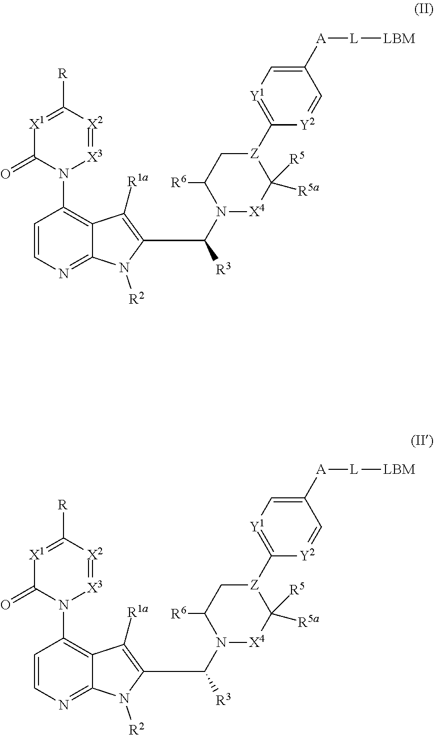
wherein R³ is C₁₋₄alkyl;
or a pharmaceutically acceptable salt or stereoisomer thereof.

4. The compound according to claim 2 having the formula (III) 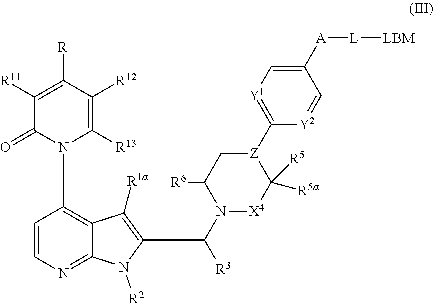
or a pharmaceutically acceptable salt or stereoisomer thereof.

5. The compound according to claim 4 having the formula (IV) 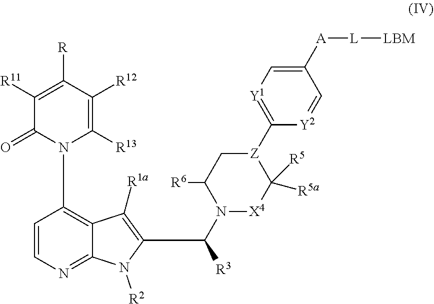
wherein R³ is C₁₋₄alkyl;
or a pharmaceutically acceptable salt or stereoisomer thereof.

6. The compound according to claim 1, wherein R is NHR⁰ and R⁰ is selected from hydrogen and C₁₋₄alkyl.

7. The compound according to claim 1 having the formula (VII) or (VIII) 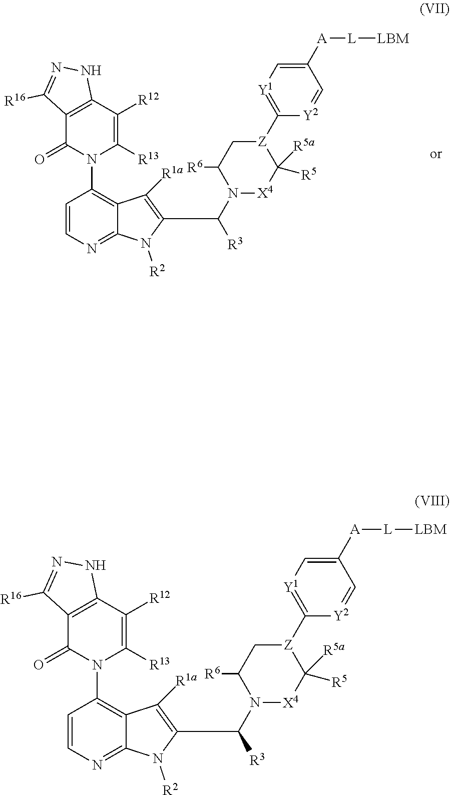
wherein R¹⁶ is selected from hydrogen and C₁₋₄alkyl, wherein said C₁₋₄alkyl may optionally be substituted one or more times with a substituent independently selected from halogen;
or a pharmaceutically acceptable salt or stereoisomer thereof.

8.-9. (canceled)

10. The compound according to claim 1, wherein the compound is of formula (IX), (IXa), or (IXb): 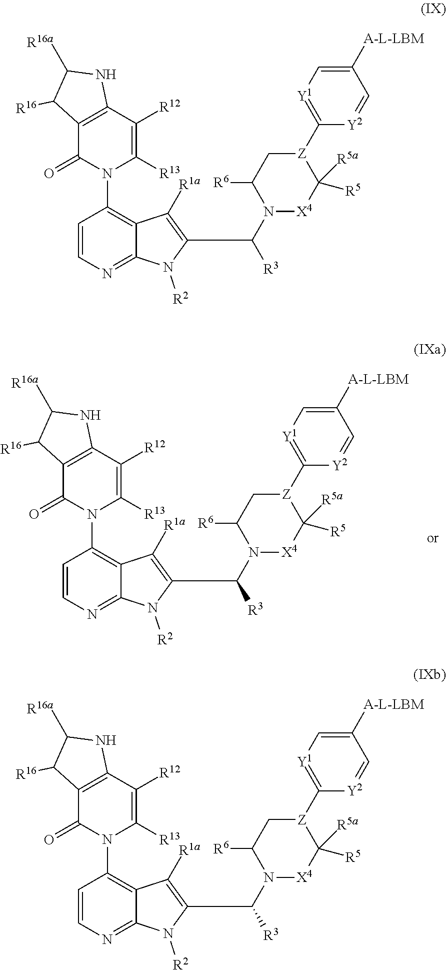
wherein R¹⁶ and R^{16a} are selected from hydrogen and C₁₋₄alkyl, wherein said C₁₋₄alkyl may optionally be substituted one or more times with a substituent independently selected from halogen;
or a pharmaceutically acceptable salt or stereoisomer thereof.

11. The compound according to claim 1, wherein X⁷ is NR²² and R²² is methyl or cyclopropyl.

12.-13. (canceled)

14. The compound according to claim 1, wherein X⁷ is CR²³R²³, and both R²³ together with the carbon they are attached to form a C₃₋₆ cycloalkyl.

15. (canceled)

16. The compound according to claim 1, wherein A-L-LBM is 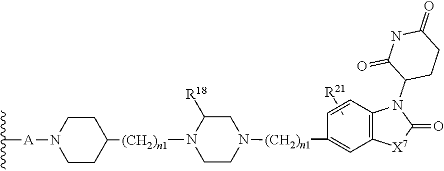
wherein A is CO and n1 is 1.

17.-18. (canceled)

19. The compound according to claim 1, wherein Z is CR¹⁰ and R⁵ and R¹⁰ together form a bond.

20. The compound according to claim 1, wherein Z is CR¹⁰ and R¹⁰ is hydrogen.

21.-23. (canceled)

24. The compound according to claim 1, wherein Y¹ is CR⁷ and Y² is CR⁸ and wherein
(a) both of R⁷ and R⁸ is C₁₋₄alkyl;
(b) both of R⁷ and R⁸ is halogen;
(c) one of R⁷ and R⁸ is C₁₋₄alkyl and the other is halogen;
(d) one of R⁷ and R⁸ is C₁₋₄alkyl and the other is hydrogen; or
(e) one of R⁷ and R⁸ is halogen and the other is hydrogen.

25. The compound according to claim 24, wherein R⁷ is halogen and R⁸ is methyl.

26. The compound according to claim 24, wherein the halogen in b) c) and e) is fluoro.

27. The compound according to claim 1, wherein R¹² and R¹³ are hydrogen; or R¹¹, R¹² and R¹³ are hydrogen.

28. (canceled)

29. A compound according to claim 1, wherein X⁴ is CR⁴R⁴ and each R⁴ is independently selected from hydrogen and C₁₋₄alkyl.

30. A compound according to claim 1, wherein R² is methyl.

31. (canceled)

32. A compound according to claim 1, wherein R³ is methyl.

33. A compound according to claim 1, wherein R^{5a} is hydrogen.

34. The compound according to claim 1, which is selected from:

| No. | Structure |
|---|---|
| 3e39 | 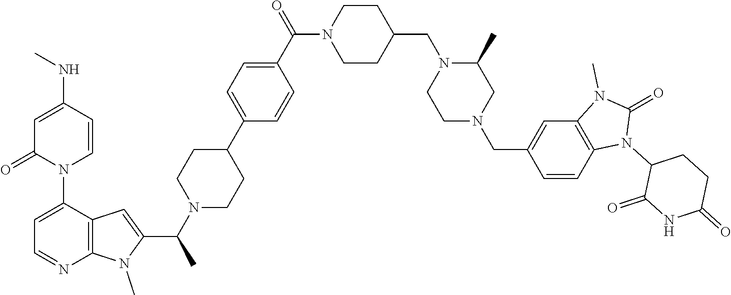 [SMILES: CNc1ccn(-c2ccnc3c2cc([C@H](C)N2CCC(c4ccc(C(=O)N5CCC(CN6CCN(Cc7ccc8c(c7)n(C)c(=O)n8C7CCC(=O)NC7=O)C[C@@H]6C)CC5)cc4)CC2)n3C)c(=O)c1] |
| 3e44 | 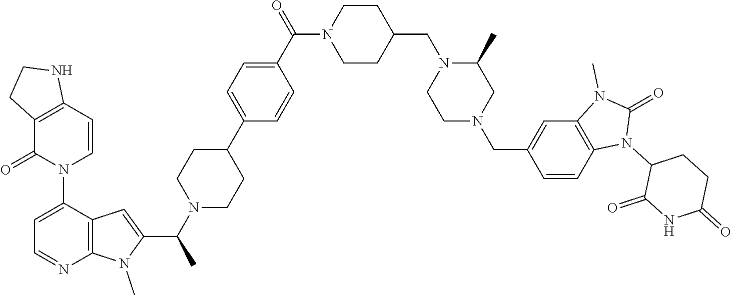 [SMILES: C[C@H]1CN(Cc2ccc3c(c2)n(C)c(=O)n3C2CCC(=O)NC2=O)CCN1CC1CCN(C(=O)c2ccc(C3CCN([C@@H](C)c4cc5c(-n6ccc7c(c6=O)CCN7)ccnc5n4C)CC3)cc2)CC1] |
| 3h10 | 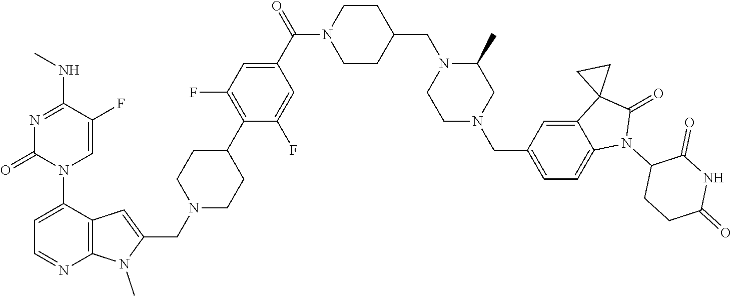 [SMILES: CNc1nc(=O)n(-c2ccnc3c2cc(CN2CCC(c4c(F)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)cc1F] |
| 3h12 | 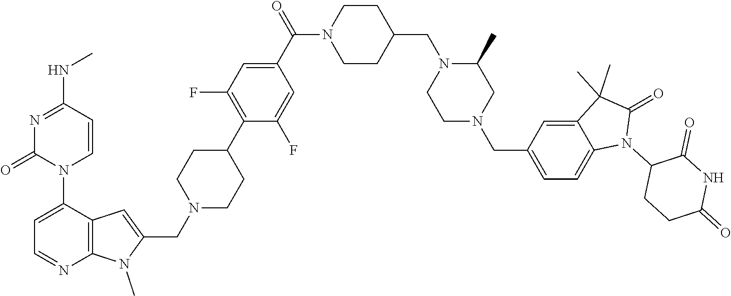 [SMILES: CNc1ccn(-c2ccnc3c2cc(CN2CCC(c4c(F)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)c(=O)n1] |
| 3u1 | 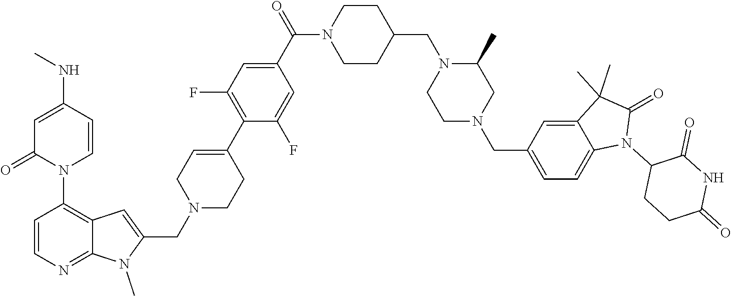 [SMILES: CNc1ccn(-c2ccnc3c2cc(CN2CC=C(c4c(F)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)c(=O)c1] |
| 3u10 | 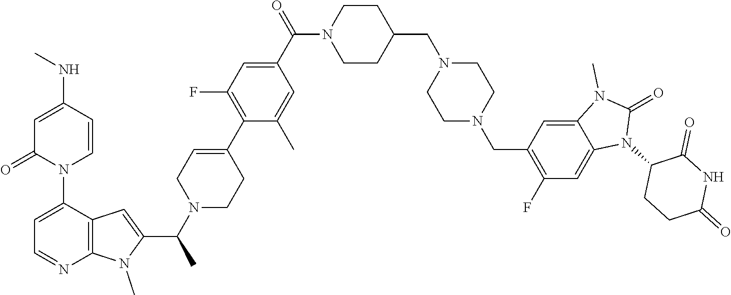 [SMILES: CNc1ccn(-c2ccnc3c2cc([C@H](C)N2CC=C(c4c(C)cc(C(=O)N5CCC(CN6CCN(Cc7cc8c(cc7F)n([C@H]7CCC(=O)NC7=O)c(=O)n8C)CC6)CC5)cc4F)CC2)n3C)c(=O)c1] |
| 3u100 and 3u99 | 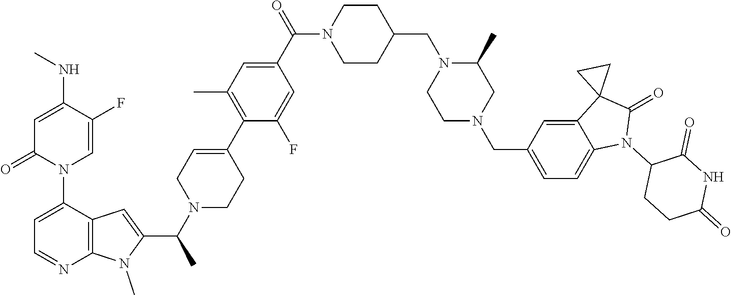 [SMILES: CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)cc1F] |
| | 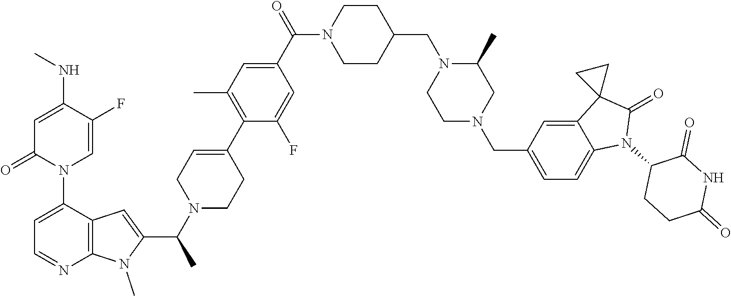 [SMILES: CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8[C@H]7CCC(=O)NC7=O)C[C@@H]6C)CC5)cc4F)CC2)n3C)cc1F] |
| 3u101 | 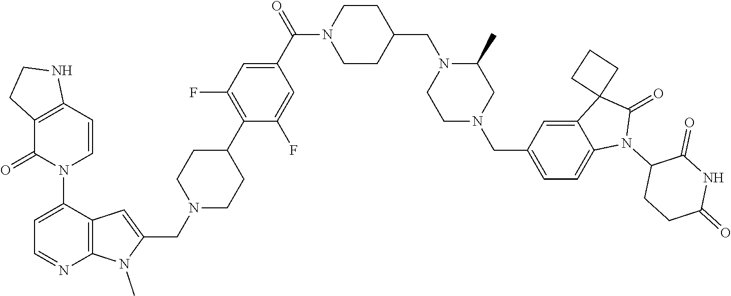 [SMILES: C[C@H]1CN(Cc2ccc3c(c2)C2(CCC2)C(=O)N3C2CCC(=O)NC2=O)CCN1CC1CCN(C(=O)c2cc(F)c(C3CCN(Cc4cc5c(-n6ccc7c(c6=O)CCN7)ccnc5n4C)CC3)c(F)c2)CC1] |
| 3u102 | 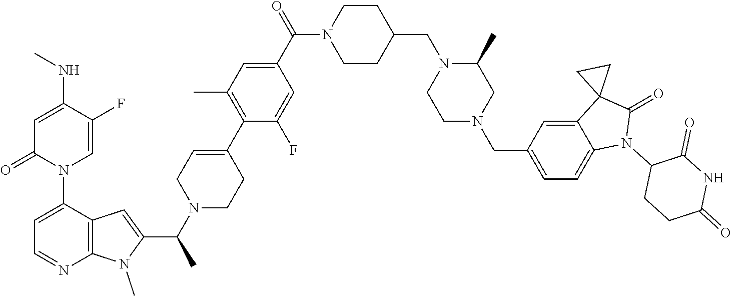 [SMILES: CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)cc1F] |
| 3u103 | 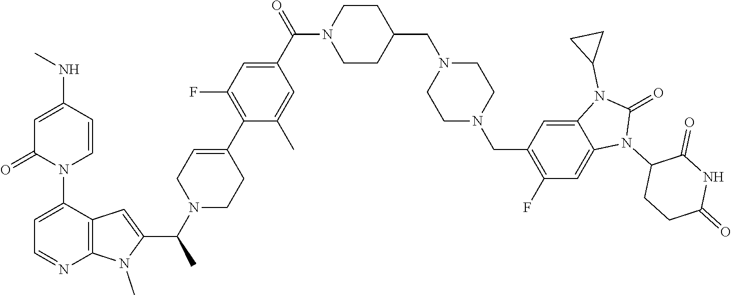 [SMILES: CNc1ccn(-c2ccnc3c2cc([C@H](C)N2CC=C(c4c(C)cc(C(=O)N5CCC(CN6CCN(Cc7cc8c(cc7F)n(C7CCC(=O)NC7=O)c(=O)n8C7CC7)CC6)CC5)cc4F)CC2)n3C)c(=O)c1] |
| 3u104 and 3u105 | 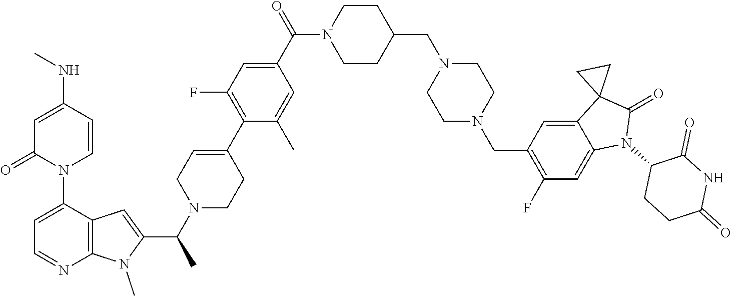 [SMILES: CNc1ccn(-c2ccnc3c2cc([C@H](C)N2CC=C(c4c(C)cc(C(=O)N5CCC(CN6CCN(Cc7cc8c(cc7F)N([C@H]7CCC(=O)NC7=O)C(=O)C87CC7)CC6)CC5)cc4F)CC2)n3C)c(=O)c1] |
| | 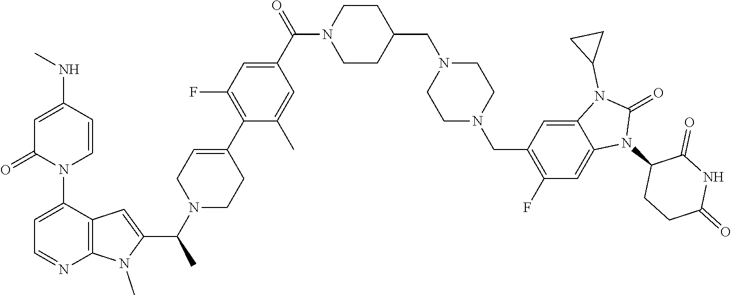 [SMILES: CNc1ccn(-c2ccnc3c2cc([C@H](C)N2CC=C(c4c(C)cc(C(=O)N5CCC(CN6CCN(Cc7cc8c(cc7F)n([C@@H]7CCC(=O)NC7=O)c(=O)n8C7CC7)CC6)CC5)cc4F)CC2)n3C)c(=O)c1] |
| 3u106 | 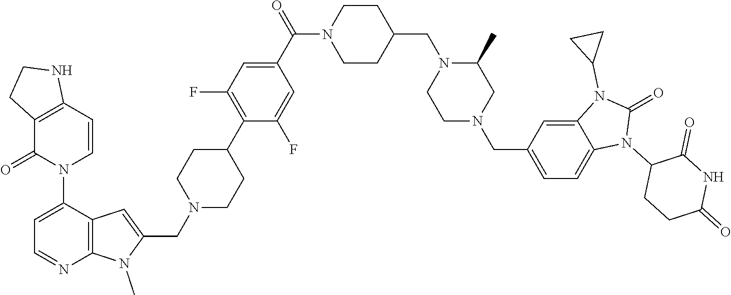 [SMILES: C[C@H]1CN(Cc2ccc3c(c2)n(C2CC2)c(=O)n3C2CCC(=O)NC2=O)CCN1CC1CCN(C(=O)c2cc(F)c(C3CCN(Cc4cc5c(-n6ccc7c(c6=O)CCN7)ccnc5n4C)CC3)c(F)c2)CC1] |
| 3u107 and 3u108 | 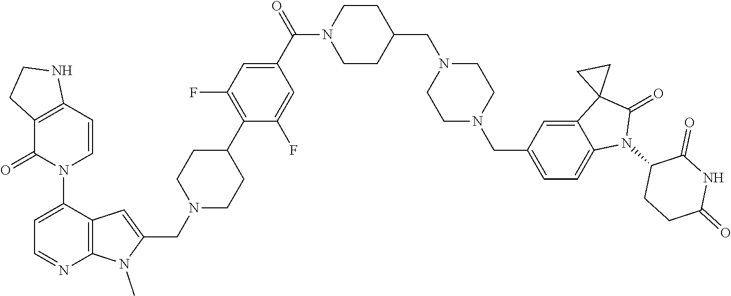 [SMILES: Cn1c(CN2CCC(c3c(F)cc(C(=O)N4CCC(CN5CCN(Cc6ccc7c(c6)C6(CC6)C(=O)N7[C@H]6CCC(=O)NC6=O)CC5)CC4)cc3F)CC2)cc2c(-n3ccc4c(c3=O)CCN4)ccnc21] |
| | 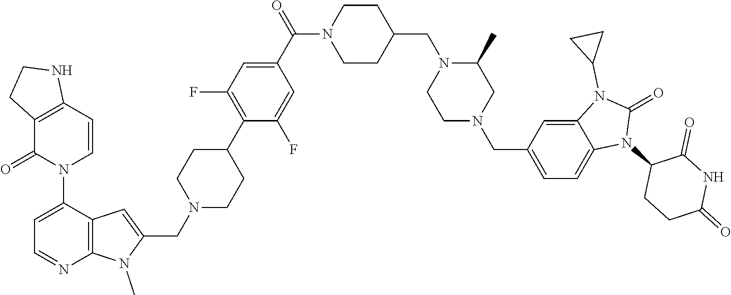 [SMILES: C[C@H]1CN(Cc2ccc3c(c2)n(C2CC2)c(=O)n3[C@@H]2CCC(=O)NC2=O)CCN1CC1CCN(C(=O)c2cc(F)c(C3CCN(Cc4cc5c(-n6ccc7c(c6=O)CCN7)ccnc5n4C)CC3)c(F)c2)CC1] |
| 3u109 | 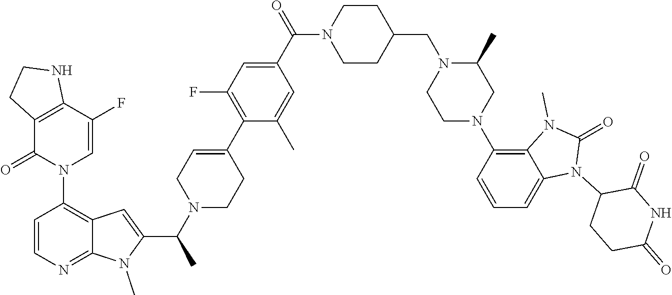 [SMILES: Cc1cc(C(=O)N2CCC(CN3CCN(c4cccc5c4n(C)c(=O)n5C4CCC(=O)NC4=O)C[C@@H]3C)CC2)cc(F)c1C1=CCN([C@@H](C)c2cc3c(-n4cc(F)c5c(c4=O)CCN5)ccnc3n2C)CC1] |
| 3u11 and 3u12 | 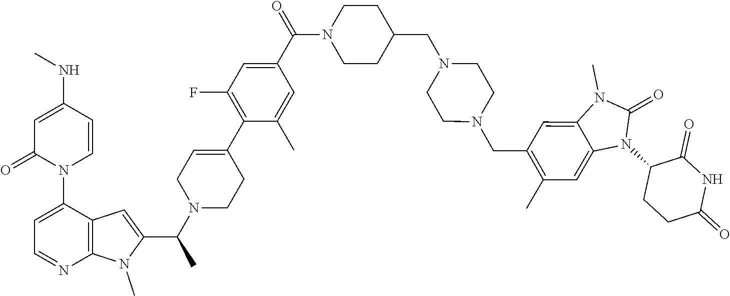 [SMILES: CNc1ccn(-c2ccnc3c2cc([C@H](C)N2CC=C(c4c(C)cc(C(=O)N5CCC(CN6CCN(Cc7cc8c(cc7C)n([C@H]7CCC(=O)NC7=O)c(=O)n8C)CC6)CC5)cc4F)CC2)n3C)c(=O)c1] |
| | 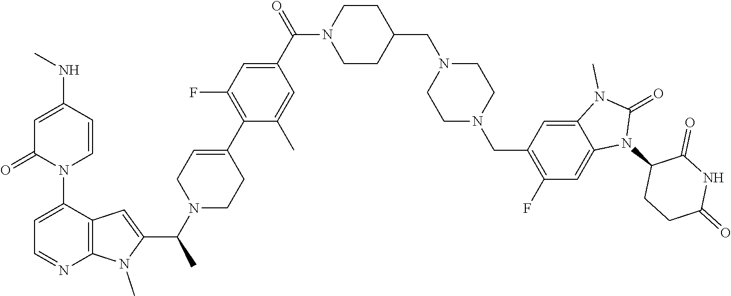 [SMILES: CNc1ccn(-c2ccnc3c2cc([C@H](C)N2CC=C(c4c(C)cc(C(=O)N5CCC(CN6CCN(Cc7cc8c(cc7F)n([C@@H]7CCC(=O)NC7=O)c(=O)n8C)CC6)CC5)cc4F)CC2)n3C)c(=O)c1] |
| 3u110 | 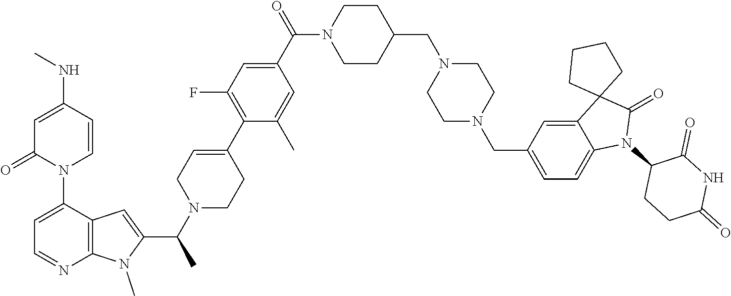 [SMILES: CNc1ccn(-c2ccnc3c2cc([C@H](C)N2CC=C(c4c(C)cc(C(=O)N5CCC(CN6CCN(Cc7ccc8c(c7)C7(CCCC7)C(=O)N8[C@@H]7CCC(=O)NC7=O)CC6)CC5)cc4F)CC2)n3C)c(=O)c1] |
| 3u111 | 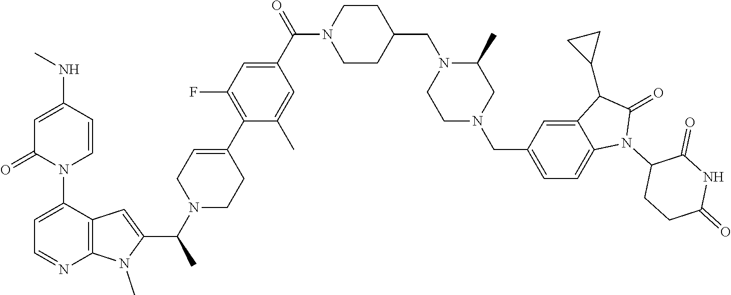 [SMILES: CNc1ccn(-c2ccnc3c2cc([C@H](C)N2CC=C(c4c(C)cc(C(=O)N5CCC(CN6CCN(Cc7ccc8c(c7)C(C7CC7)C(=O)N8C7CCC(=O)NC7=O)C[C@@H]6C)CC5)cc4F)CC2)n3C)c(=O)c1] |
| 3u112 and 3u113 | 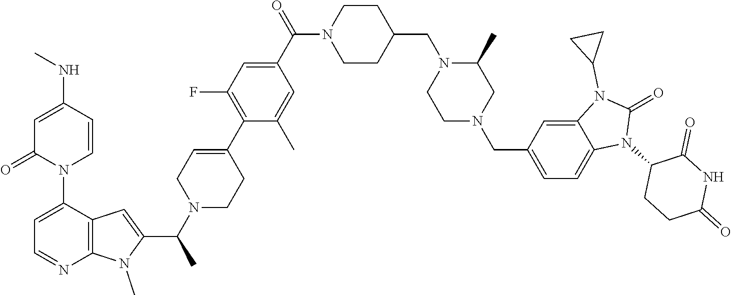 [SMILES: CNc1ccn(-c2ccnc3c2cc([C@H](C)N2CC=C(c4c(C)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)c(=O)c1] |
| | 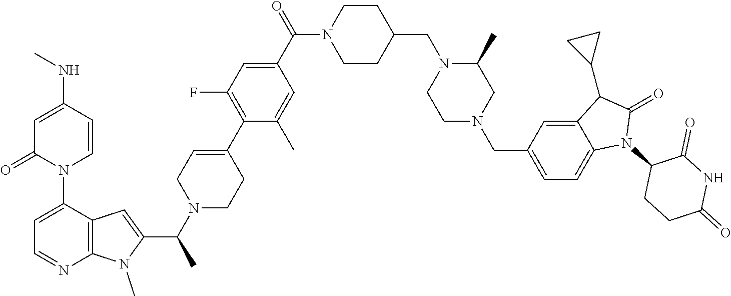 [SMILES: CNc1ccn(-c2ccnc3c2cc([C@H](C)N2CC=C(c4c(C)cc(C(=O)N5CCC(CN6CCN(Cc7ccc8c(c7)C(C7CC7)C(=O)N8[C@@H]7CCC(=O)NC7=O)C[C@@H]6C)CC5)cc4F)CC2)n3C)c(=O)c1] |
| 3u114 | 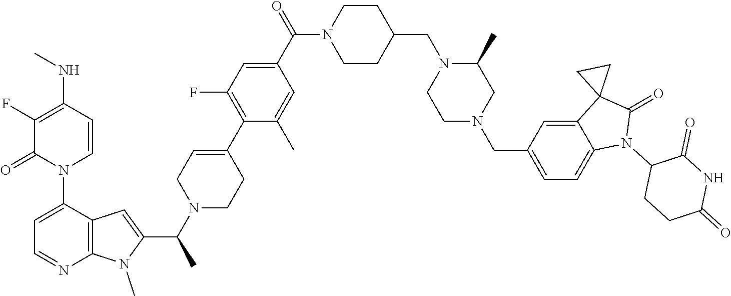 [SMILES: CNc1ccn(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)c(=O)c1F] |
| 3u115 | 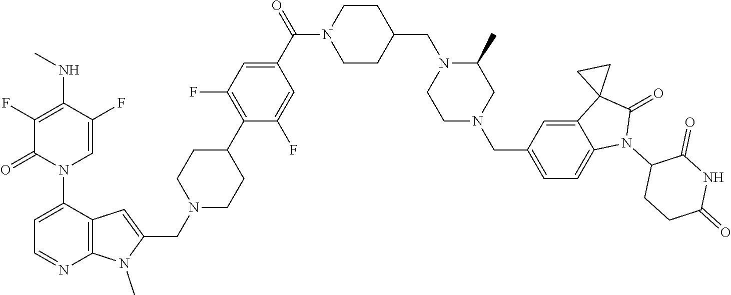 [SMILES: CNc1c(F)cn(-c2ccnc3c2cc(CN2CCC(c4c(F)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)c(=O)c1F] |
| 3u116 | 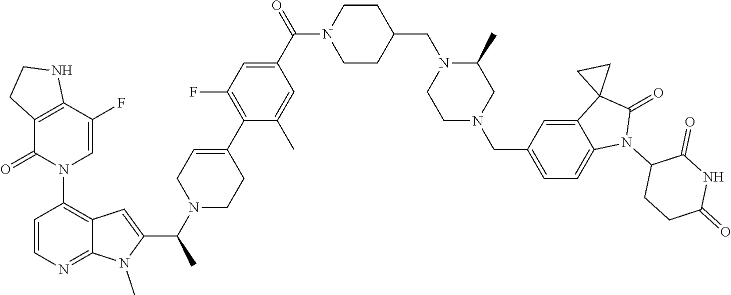 [SMILES: Cc1cc(C(=O)N2CCC(CN3CCN(Cc4ccc5c(c4)C4(CC4)C(=O)N5C4CCC(=O)NC4=O)C[C@@H]3C)CC2)cc(F)c1C1=CCN([C@@H](C)c2cc3c(-n4cc(F)c5c(c4=O)CCN5)ccnc3n2C)CC1] |
| 3u117 | 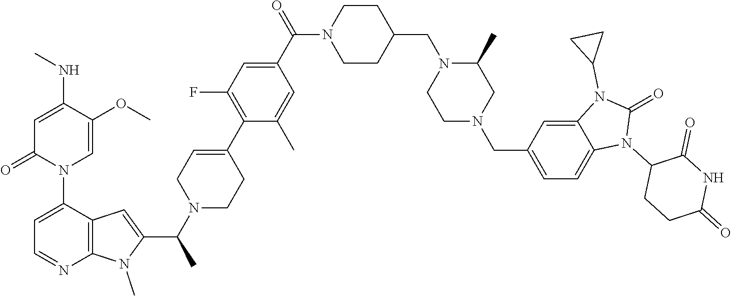 [SMILES: CNc1cc(=O)n(-c2ccnc3c2cc([C@H](C)N2CC=C(c4c(C)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)cc1OC] |
| 3u118 | 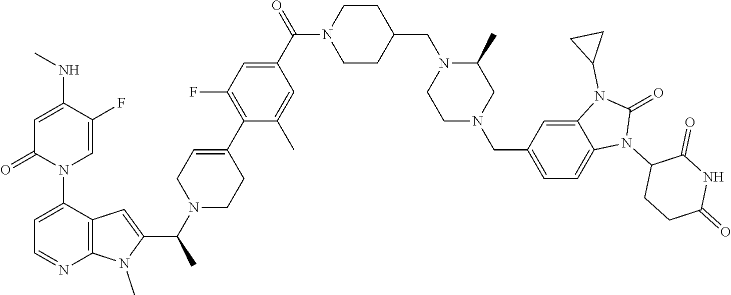 [SMILES: CNc1cc(=O)n(-c2ccnc3c2cc([C@H](C)N2CC=C(c4c(C)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)cc1F] |
| 3u119 and 3u120 | 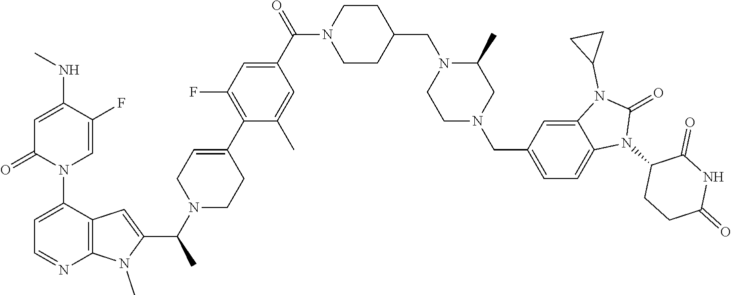 [SMILES: CNc1cc(=O)n(-c2ccnc3c2cc([C@H](C)N2CC=C(c4c(C)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)cc1F] |
| | 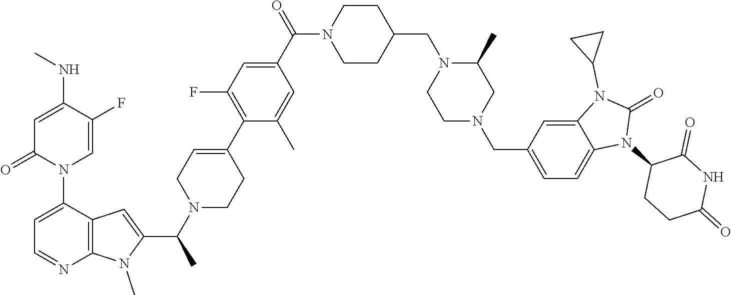 [SMILES: CNc1cc(=O)n(-c2ccnc3c2cc([C@H](C)N2CC=C(c4c(C)cc(C(=O)N5CCC(CN6CCN(Cc7ccc8c(c7)n(C7CC7)c(=O)n8[C@@H]7CCC(=O)NC7=O)C[C@@H]6C)CC5)cc4F)CC2)n3C)cc1F] |
| 3u121 | 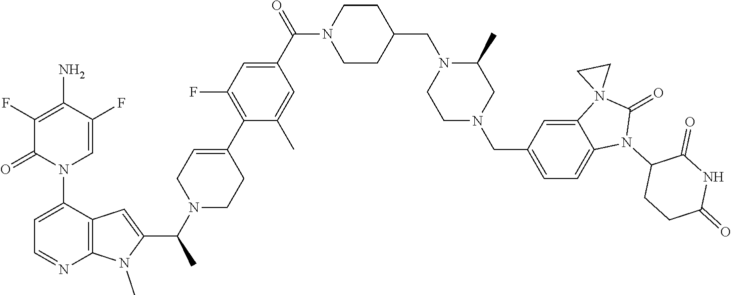 |
| 3u122 | 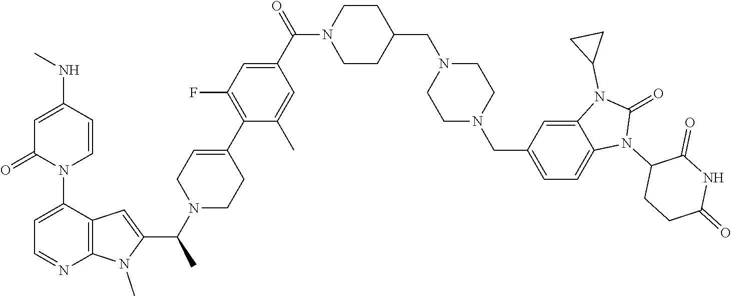 [SMILES: CNc1ccn(-c2ccnc3c2cc([C@H](C)N2CC=C(c4c(C)cc(C(=O)N5CCC(CN6CCN(Cc7ccc8c(c7)n(C7CC7)c(=O)n8C7CCC(=O)NC7=O)CC6)CC5)cc4F)CC2)n3C)c(=O)c1] |
| 3u123 and 3u124 | 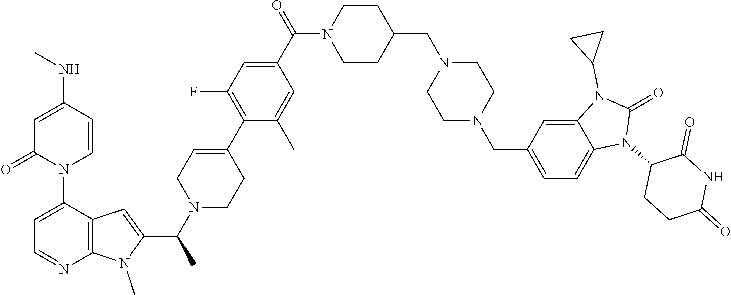 [SMILES: CNc1ccn(-c2ccnc3c2cc([C@H](C)N2CC=C(c4c(C)cc(C(=O)N5CCC(CN6CCN(Cc7ccc8c(c7)n(C7CC7)c(=O)n8[C@H]7CCC(=O)NC7=O)CC6)CC5)cc4F)CC2)n3C)c(=O)c1] |
| | 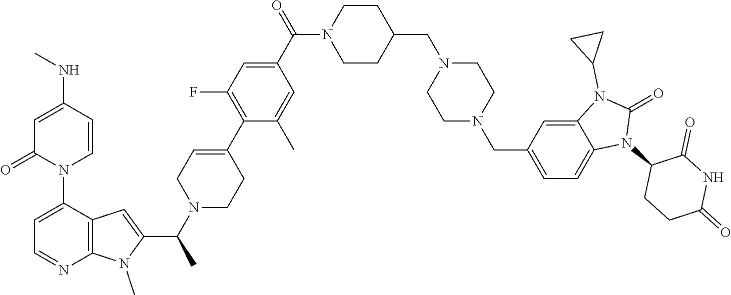 [SMILES: CNc1ccn(-c2ccnc3c2cc([C@H](C)N2CC=C(c4c(C)cc(C(=O)N5CCC(CN6CCN(Cc7ccc8c(c7)n(C7CC7)c(=O)n8[C@@H]7CCC(=O)NC7=O)CC6)CC5)cc4F)CC2)n3C)c(=O)c1] |
| 3u125 | 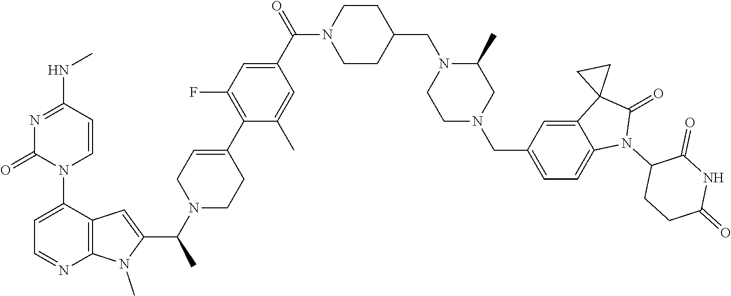 [SMILES: CNc1ccn(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)c(=O)n1] |
| 3u126 | 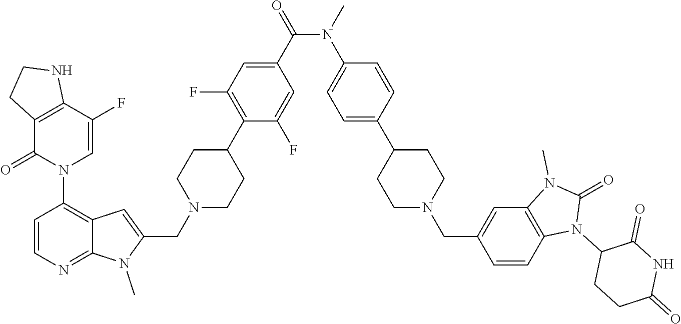 [SMILES: CN(C(=O)c1cc(F)c(C2CCN(Cc3cc4c(-n5cc(F)c6c(c5=O)CCN6)ccnc4n3C)CC2)c(F)c1)c1ccc(C2CCN(Cc3ccc4c(c3)n(C)c(=O)n4C3CCC(=O)NC3=O)CC2)cc1] |
| 3u127 | 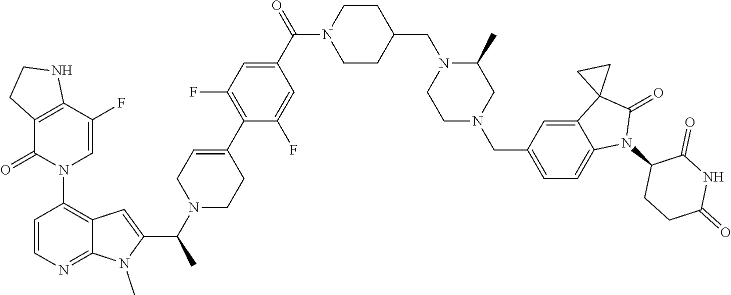 [SMILES: C[C@H]1CN(Cc2ccc3c(c2)C2(CC2)C(=O)N3[C@@H]2CCC(=O)NC2=O)CCN1CC1CCN(C(=O)c2cc(F)c(C3=CCN([C@@H](C)c4cc5c(-n6cc(F)c7c(c6=O)CCN7)ccnc5n4C)CC3)c(F)c2)CC1] |
| 3u128 | 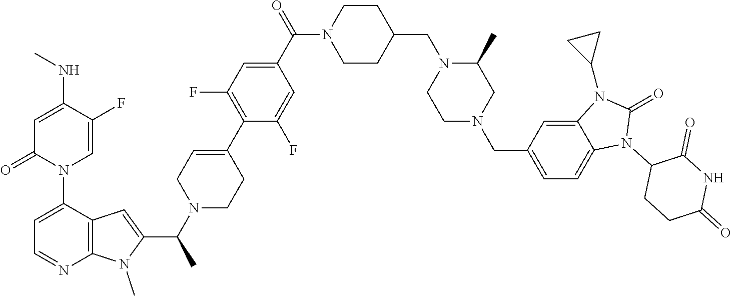 [SMILES: CNc1cc(=O)n(-c2ccnc3c2cc([C@H](C)N2CC=C(c4c(F)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)cc1F] |
| 3u129 and 3u130 | 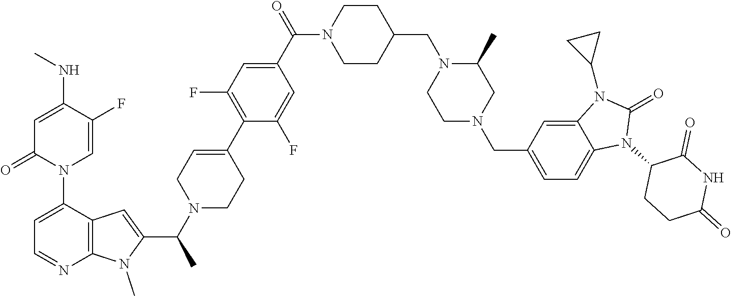 [SMILES: CNc1cc(=O)n(-c2ccnc3c2cc([C@H](C)N2CC=C(c4c(F)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)cc1F] |
| | 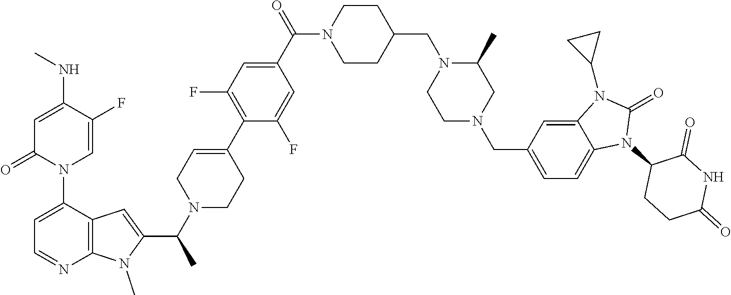 [SMILES: CNc1cc(=O)n(-c2ccnc3c2cc([C@H](C)N2CC=C(c4c(F)cc(C(=O)N5CCC(CN6CCN(Cc7ccc8c(c7)n(C7CC7)c(=O)n8[C@@H]7CCC(=O)NC7=O)C[C@@H]6C)CC5)cc4F)CC2)n3C)cc1F] |
| 3u13 | 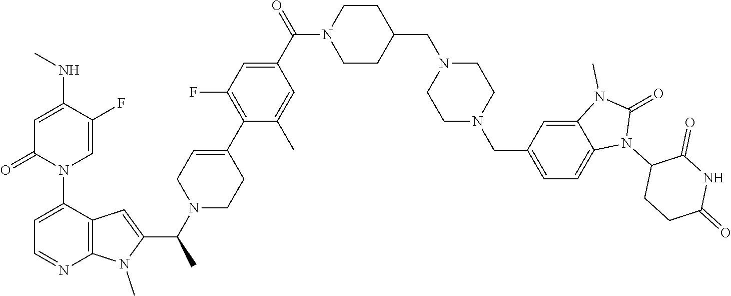 [SMILES: CNc1cc(=O)n(-c2ccnc3c2cc([C@H](C)N2CC=C(c4c(C)cc(C(=O)N5CCC(CN6CCN(Cc7ccc8c(c7)n(C)c(=O)n8C7CCC(=O)NC7=O)CC6)CC5)cc4F)CC2)n3C)cc1F] |
| 3u131 | 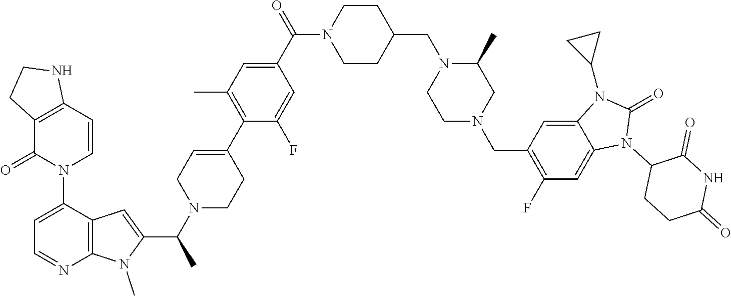 [SMILES: Cc1cc(C(=O)N2CCC(CN3CCN(Cc4cc5c(cc4F)n(C4CCC(=O)NC4=O)c(=O)n5C4CC4)C[C@@H]3C)CC2)cc(F)c1C1=CCN([C@@H](C)c2cc3c(-n4ccc5c(c4=O)CCN5)ccnc3n2C)CC1] |
| 3u132 and 3u133 | 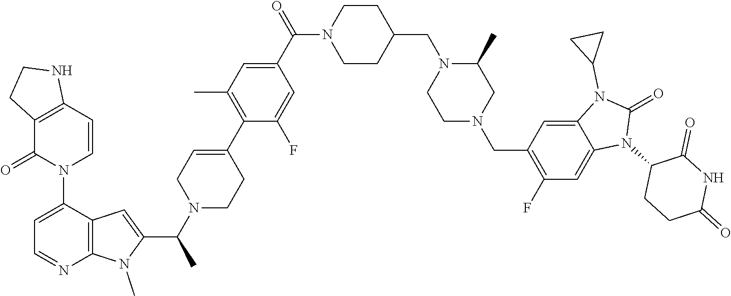 [SMILES: Cc1cc(C(=O)N2CCC(CN3CCN(Cc4cc5c(cc4F)n([C@H]4CCC(=O)NC4=O)c(=O)n5C4CC4)C[C@@H]3C)CC2)cc(F)c1C1=CCN([C@@H](C)c2cc3c(-n4ccc5c(c4=O)CCN5)ccnc3n2C)CC1] |
| | 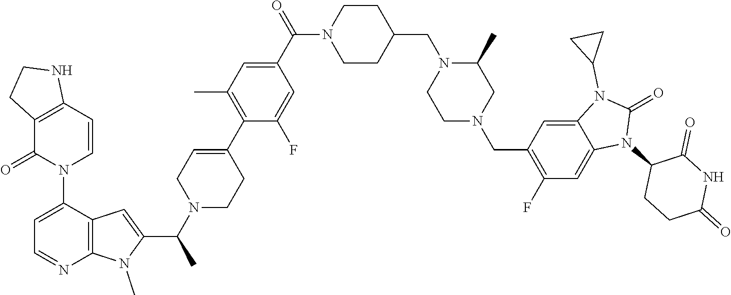 [SMILES: Cc1cc(C(=O)N2CCC(CN3CCN(Cc4cc5c(cc4F)n([C@@H]4CCC(=O)NC4=O)c(=O)n5C4CC4)C[C@@H]3C)CC2)cc(F)c1C1=CCN([C@@H](C)c2cc3c(-n4ccc5c(c4=O)CCN5)ccnc3n2C)CC1] |
| 3u134 | 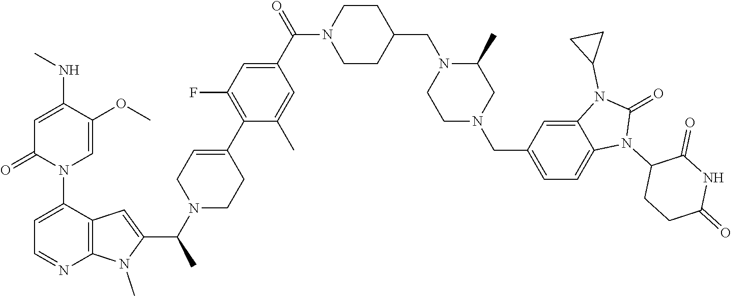 [SMILES: CNc1cc(=O)n(-c2ccnc3c2cc([C@H](C)N2CC=C(c4c(C)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)cc1OC] |
| 3u135 | 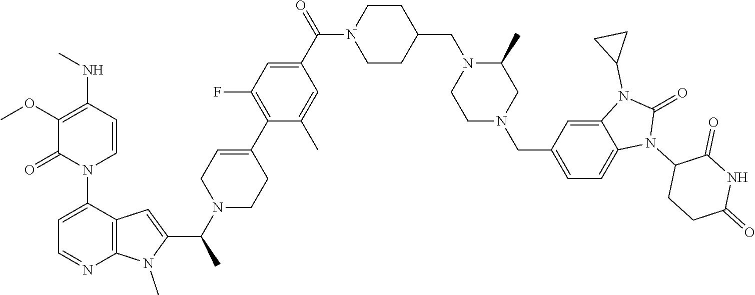 [SMILES: CNc1ccn(-c2ccnc3c2cc([C@H](C)N2CC=C(c4c(C)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)c(=O)c1OC] |
| 3u136 | 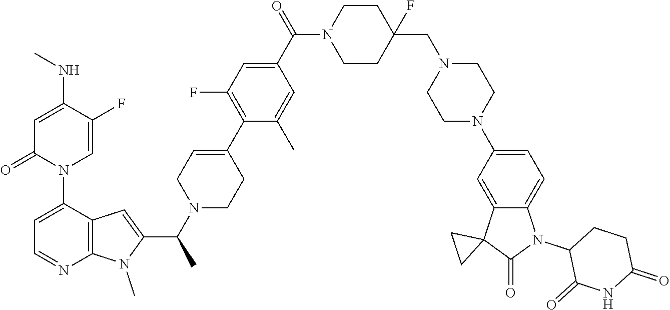 [SMILES: CNc1cc(=O)n(-c2ccnc3c2cc([C@H](C)N2CC=C(c4c(C)cc(C(=O)N5CCC(F)(CN6CCN(c7ccc8c(c7)C7(CC7)C(=O)N8C7CCC(=O)NC7=O)CC6)CC5)cc4F)CC2)n3C)cc1F] |
| 3u137 | 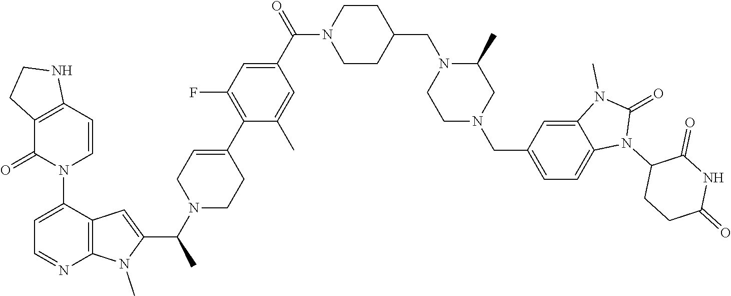 [SMILES: Cc1cc(C(=O)N2CCC(CN3CCN(Cc4ccc5c(c4)n(C)c(=O)n5C4CCC(=O)NC4=O)C[C@@H]3C)CC2)cc(F)c1C1=CCN([C@@H](C)c2cc3c(-n4ccc5c(c4=O)CCN5)ccnc3n2C)CC1] |
| 3u138 and 3u139 | 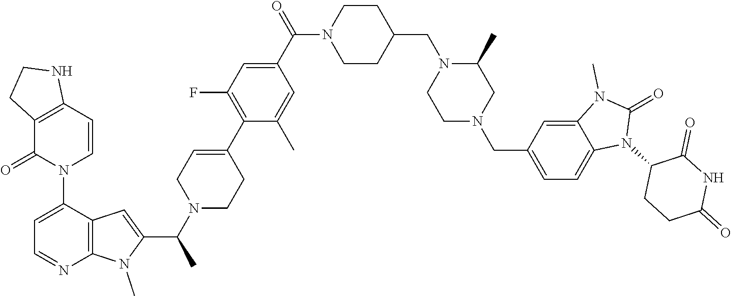 [SMILES: Cc1cc(C(=O)N2CCC(CN3CCN(Cc4ccc5c(c4)n(C)c(=O)n5[C@H]4CCC(=O)NC4=O)C[C@@H]3C)CC2)cc(F)c1C1=CCN([C@@H](C)c2cc3c(-n4ccc5c(c4=O)CCN5)ccnc3n2C)CC1] |
| | 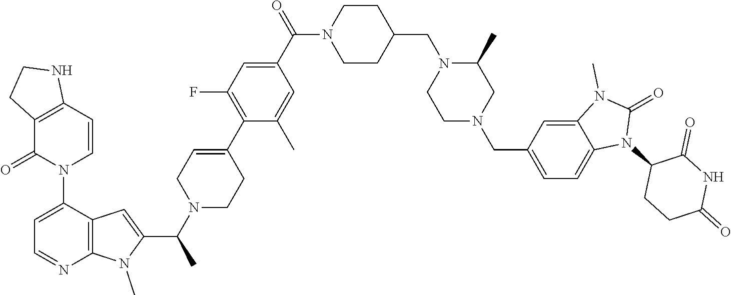 [SMILES: Cc1cc(C(=O)N2CCC(CN3CCN(Cc4ccc5c(c4)n(C)c(=O)n5[C@@H]4CCC(=O)NC4=O)C[C@@H]3C)CC2)cc(F)c1C1=CCN([C@@H](C)c2cc3c(-n4ccc5c(c4=O)CCN5)ccnc3n2C)CC1] |
| 3u14 and 3u15 | 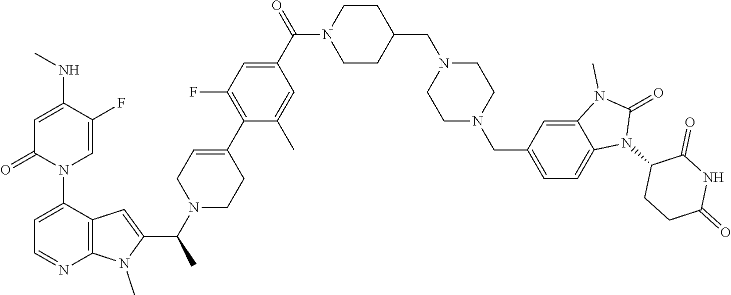 [SMILES: CNc1cc(=O)n(-c2ccnc3c2cc([C@H](C)N2CC=C(c4c(C)cc(C(=O)N5CCC(CN6CCN(Cc7ccc8c(c7)n(C)c(=O)n8[C@H]7CCC(=O)NC7=O)CC6)CC5)cc4F)CC2)n3C)cc1F] |
| | 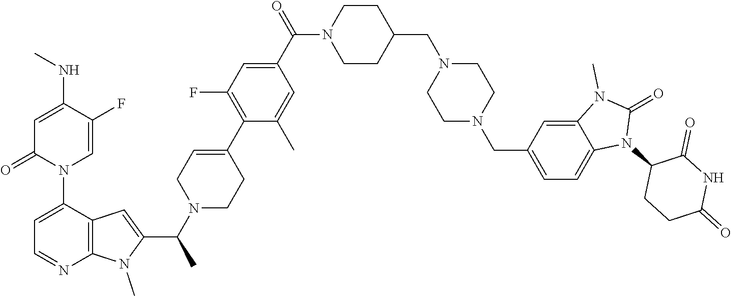 [SMILES: CNc1cc(=O)n(-c2ccnc3c2cc([C@H](C)N2CC=C(c4c(C)cc(C(=O)N5CCC(CN6CCN(Cc7ccc8c(c7)n(C)c(=O)n8[C@@H]7CCC(=O)NC7=O)CC6)CC5)cc4F)CC2)n3C)cc1F] |
| 3u140 | 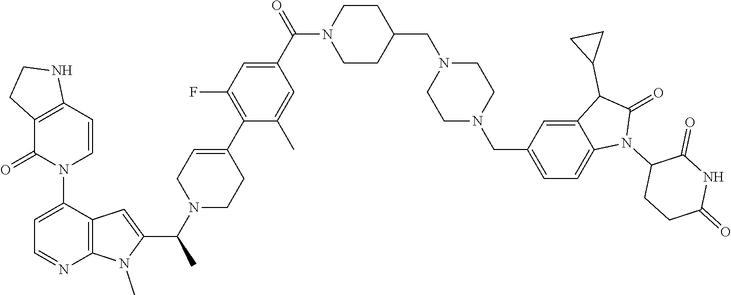 [SMILES: Cc1cc(C(=O)N2CCC(CN3CCN(Cc4ccc5c(c4)C(C4CC4)C(=O)N5C4CCC(=O)NC4=O)CC3)CC2)cc(F)c1C1=CCN([C@@H](C)c2cc3c(-n4ccc5c(c4=O)CCN5)ccnc3n2C)CC1] |
| 3u141 and 3u142 | 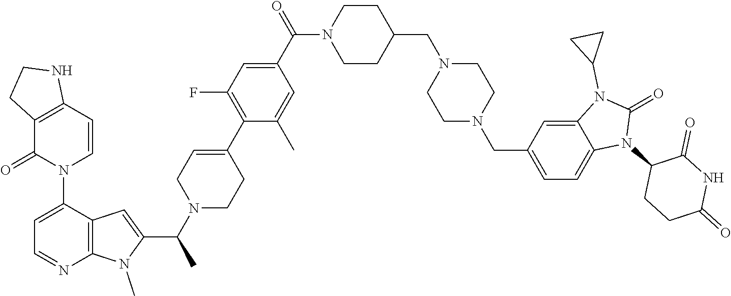 [SMILES: Cc1cc(C(=O)N2CCC(CN3CCN(Cc4ccc5c(c4)n(C4CC4)c(=O)n5[C@@H]4CCC(=O)NC4=O)CC3)CC2)cc(F)c1C1=CCN([C@@H](C)c2cc3c(-n4ccc5c(c4=O)CCN5)ccnc3n2C)CC1] |
| | 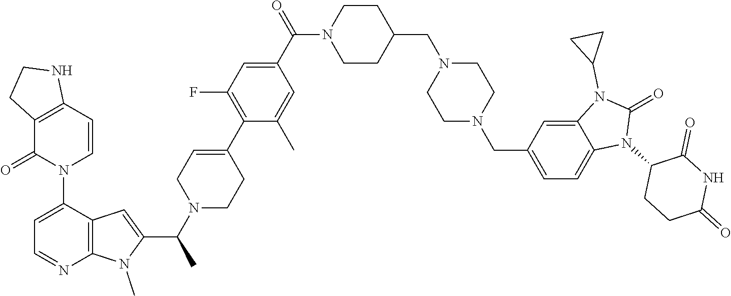 [SMILES: Cc1cc(C(=O)N2CCC(CN3CCN(Cc4ccc5c(c4)n(C4CC4)c(=O)n5[C@H]4CCC(=O)NC4=O)CC3)CC2)cc(F)c1C1=CCN([C@@H](C)c2cc3c(-n4ccc5c(c4=O)CCN5)ccnc3n2C)CC1] |
| 3u143 | 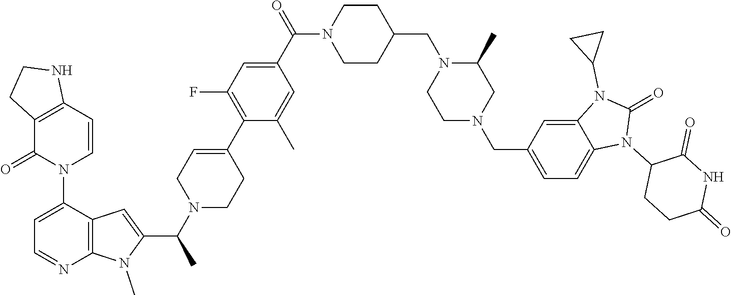 [SMILES: Cc1cc(C(=O)N2CCC(CN3CCN(Cc4ccc5c(c4)n(C4CC4)c(=O)n5C4CCC(=O)NC4=O)C[C@@H]3C)CC2)cc(F)c1C1=CCN([C@@H](C)c2cc3c(-n4ccc5c(c4=O)CCN5)ccnc3n2C)CC1] |
| 3u144 and 3u145 | 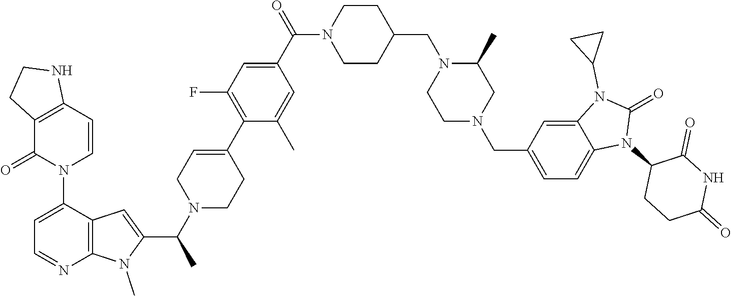 [SMILES: Cc1cc(C(=O)N2CCC(CN3CCN(Cc4ccc5c(c4)n(C4CC4)c(=O)n5[C@@H]4CCC(=O)NC4=O)C[C@@H]3C)CC2)cc(F)c1C1=CCN([C@@H](C)c2cc3c(-n4ccc5c(c4=O)CCN5)ccnc3n2C)CC1] |
| | 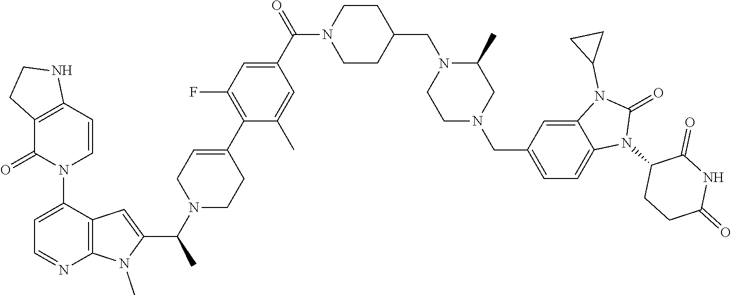 [SMILES: Cc1cc(C(=O)N2CCC(CN3CCN(Cc4ccc5c(c4)n(C4CC4)c(=O)n5[C@H]4CCC(=O)NC4=O)C[C@@H]3C)CC2)cc(F)c1C1=CCN([C@@H](C)c2cc3c(-n4ccc5c(c4=O)CCN5)ccnc3n2C)CC1] |
| 3u146 | 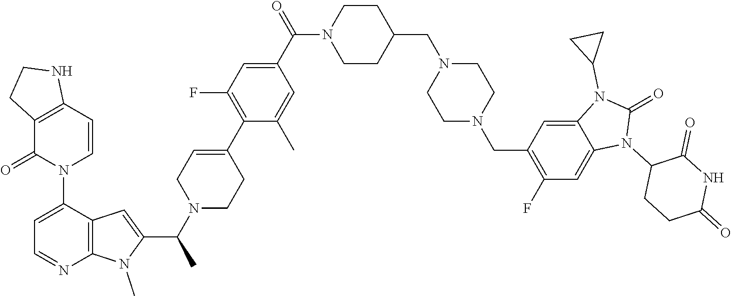 [SMILES: Cc1cc(C(=O)N2CCC(CN3CCN(Cc4cc5c(cc4F)n(C4CCC(=O)NC4=O)c(=O)n5C4CC4)CC3)CC2)cc(F)c1C1=CCN([C@@H](C)c2cc3c(-n4ccc5c(c4=O)CCN5)ccnc3n2C)CC1] |
| 3u147 and 3u148 | 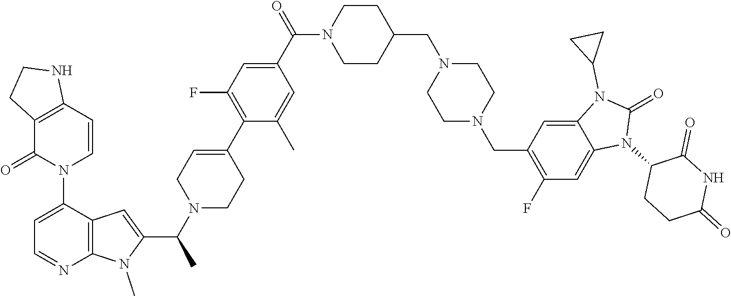 [SMILES: Cc1cc(C(=O)N2CCC(CN3CCN(Cc4cc5c(cc4F)n([C@H]4CCC(=O)NC4=O)c(=O)n5C4CC4)CC3)CC2)cc(F)c1C1=CCN([C@@H](C)c2cc3c(-n4ccc5c(c4=O)CCN5)ccnc3n2C)CC1] |
| | 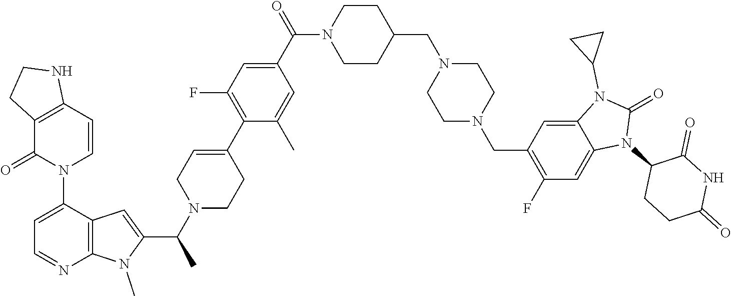 [SMILES: Cc1cc(C(=O)N2CCC(CN3CCN(Cc4cc5c(cc4F)n([C@@H]4CCC(=O)NC4=O)c(=O)n5C4CC4)CC3)CC2)cc(F)c1C1=CCN([C@@H](C)c2cc3c(-n4ccc5c(c4=O)CCN5)ccnc3n2C)CC1] |
| 3u149 | 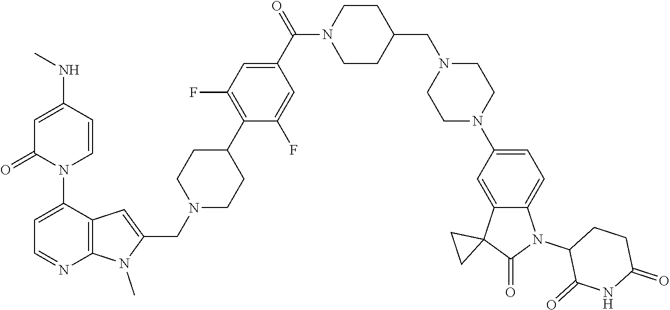 [SMILES: CNc1ccn(-c2ccnc3c2cc(CN2CCC(c4c(F)cc(C(=O)N5CCC(CN6CCN(c7ccc8c(c7)C7(CC7)C(=O)N8C7CCC(=O)NC7=O)CC6)CC5)cc4F)CC2)n3C)c(=O)c1] |
| 3u150 and 3u151 | 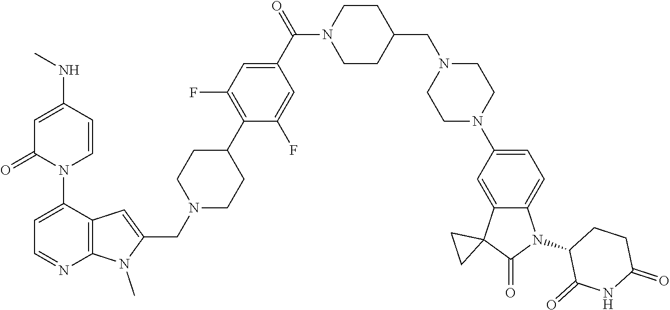 [SMILES: CNc1ccn(-c2ccnc3c2cc(CN2CCC(c4c(F)cc(C(=O)N5CCC(CN6CCN(c7ccc8c(c7)C7(CC7)C(=O)N8[C@@H]7CCC(=O)NC7=O)CC6)CC5)cc4F)CC2)n3C)c(=O)c1] |
| | 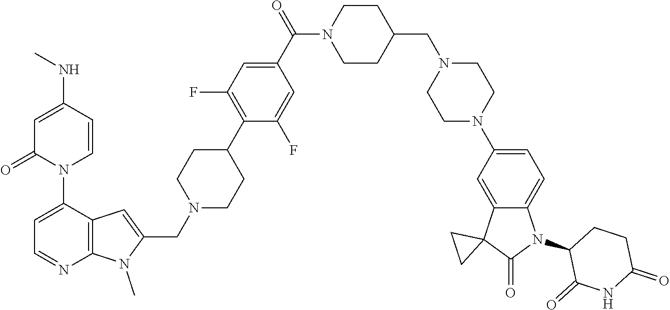 [SMILES: CNc1ccn(-c2ccnc3c2cc(CN2CCC(c4c(F)cc(C(=O)N5CCC(CN6CCN(c7ccc8c(c7)C7(CC7)C(=O)N8[C@H]7CCC(=O)NC7=O)CC6)CC5)cc4F)CC2)n3C)c(=O)c1] |
| 3u152 | 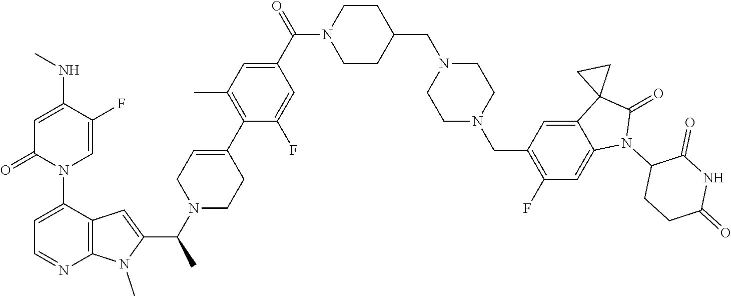 [SMILES: CNc1cc(=O)n(-c2ccnc3c2cc([C@H](C)N2CC=C(c4c(C)cc(C(=O)N5CCC(CN6CCN(Cc7cc8c(cc7F)N(C7CCC(=O)NC7=O)C(=O)C87CC7)CC6)CC5)cc4F)CC2)n3C)cc1F] |
| 3u153 and 3u154 | 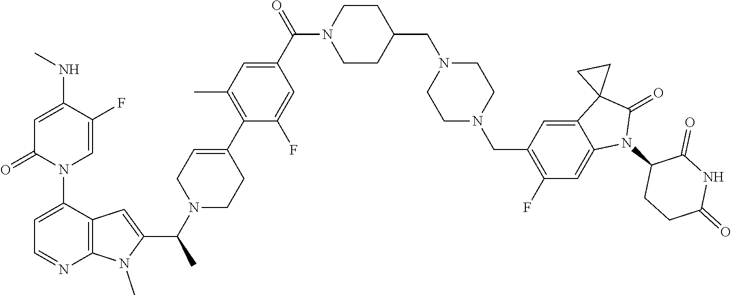 [SMILES: CNc1cc(=O)n(-c2ccnc3c2cc([C@H](C)N2CC=C(c4c(C)cc(C(=O)N5CCC(CN6CCN(Cc7cc8c(cc7F)N([C@@H]7CCC(=O)NC7=O)C(=O)C87CC7)CC6)CC5)cc4F)CC2)n3C)cc1F] |
| | 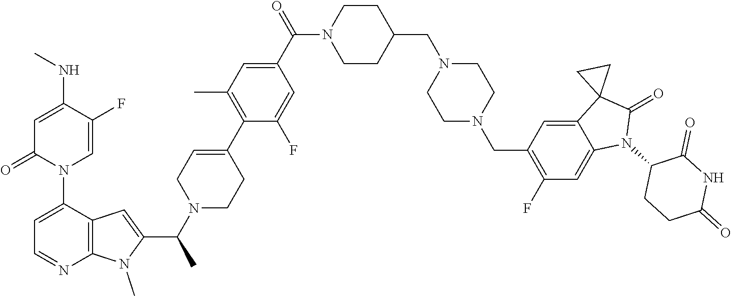 [SMILES: CNc1cc(=O)n(-c2ccnc3c2cc([C@H](C)N2CC=C(c4c(C)cc(C(=O)N5CCC(CN6CCN(Cc7cc8c(cc7F)N([C@H]7CCC(=O)NC7=O)C(=O)C87CC7)CC6)CC5)cc4F)CC2)n3C)cc1F] |
| 3u155 | 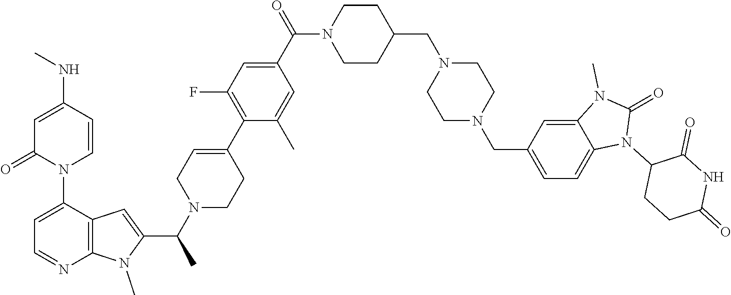 [SMILES: CNc1ccn(-c2ccnc3c2cc([C@H](C)N2CC=C(c4c(C)cc(C(=O)N5CCC(CN6CCN(Cc7ccc8c(c7)n(C)c(=O)n8C7CCC(=O)NC7=O)CC6)CC5)cc4F)CC2)n3C)c(=O)c1] |
| 3u156 and 3u157 | 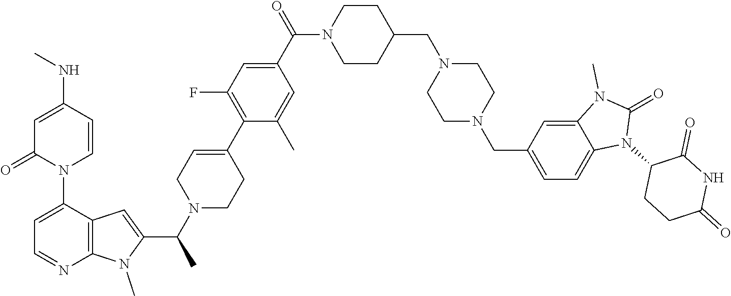 [SMILES: CNc1ccn(-c2ccnc3c2cc([C@H](C)N2CC=C(c4c(C)cc(C(=O)N5CCC(CN6CCN(Cc7ccc8c(c7)n(C)c(=O)n8[C@H]7CCC(=O)NC7=O)CC6)CC5)cc4F)CC2)n3C)c(=O)c1] |
| | 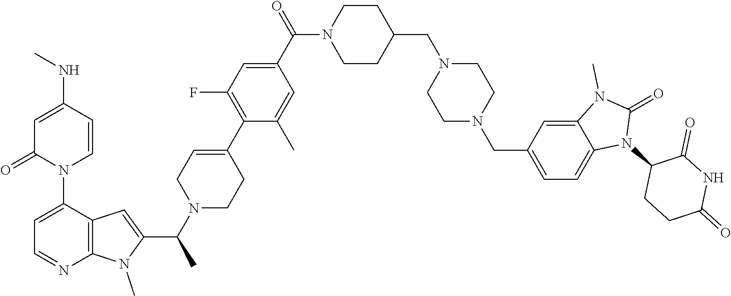 [SMILES: CNc1ccn(-c2ccnc3c2cc([C@H](C)N2CC=C(c4c(C)cc(C(=O)N5CCC(CN6CCN(Cc7ccc8c(c7)n(C)c(=O)n8[C@@H]7CCC(=O)NC7=O)CC6)CC5)cc4F)CC2)n3C)c(=O)c1] |
| 3u158 | 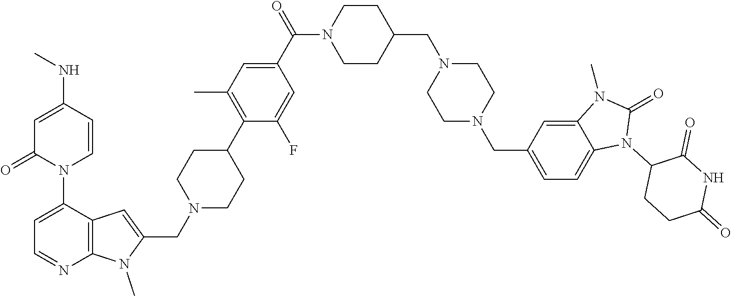 [SMILES: CNc1ccn(-c2ccnc3c2cc(CN2CCC(c4c(C)cc(C(=O)N5CCC(CN6CCN(Cc7ccc8c(c7)n(C)c(=O)n8C7CCC(=O)NC7=O)CC6)CC5)cc4F)CC2)n3C)c(=O)c1] |
| 3u159 and 3u160 | 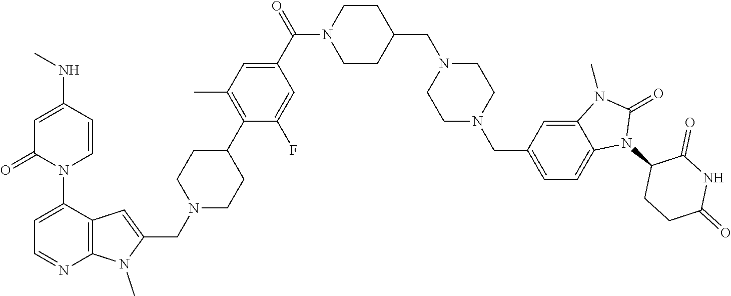 [SMILES: CNc1ccn(-c2ccnc3c2cc(CN2CCC(c4c(C)cc(C(=O)N5CCC(CN6CCN(Cc7ccc8c(c7)n(C)c(=O)n8[C@@H]7CCC(=O)NC7=O)CC6)CC5)cc4F)CC2)n3C)c(=O)c1] |
| | 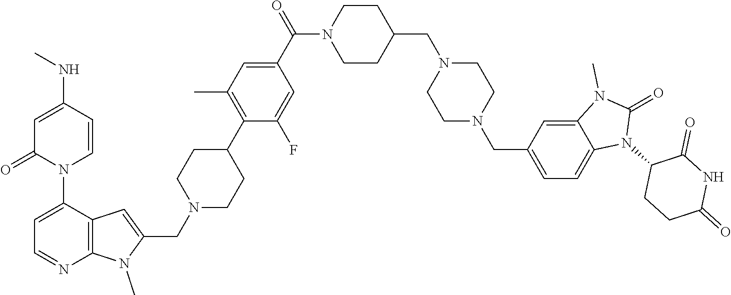 [SMILES: CNc1ccn(-c2ccnc3c2cc(CN2CCC(c4c(C)cc(C(=O)N5CCC(CN6CCN(Cc7ccc8c(c7)n(C)c(=O)n8[C@H]7CCC(=O)NC7=O)CC6)CC5)cc4F)CC2)n3C)c(=O)c1] |
| 3u16 | 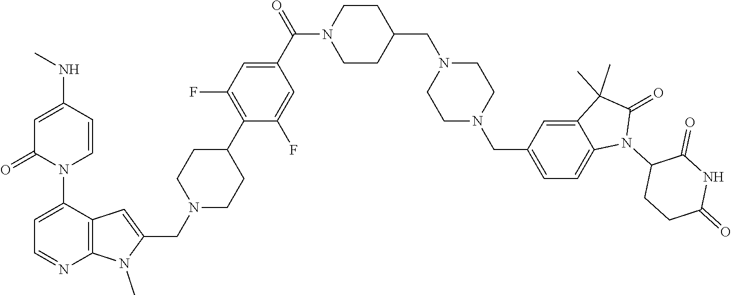 [SMILES: CNc1ccn(-c2ccnc3c2cc(CN2CCC(c4c(F)cc(C(=O)N5CCC(CN6CCN(Cc7ccc8c(c7)C(C)(C)C(=O)N8C7CCC(=O)NC7=O)CC6)CC5)cc4F)CC2)n3C)c(=O)c1] |
| 3u161 and 3u162 | 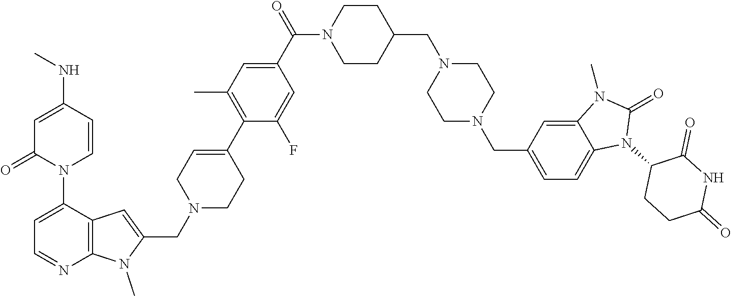 [SMILES: CNc1ccn(-c2ccnc3c2cc(CN2CC=C(c4c(C)cc(C(=O)N5CCC(CN6CCN(Cc7ccc8c(c7)n(C)c(=O)n8[C@H]7CCC(=O)NC7=O)CC6)CC5)cc4F)CC2)n3C)c(=O)c1] |
| | 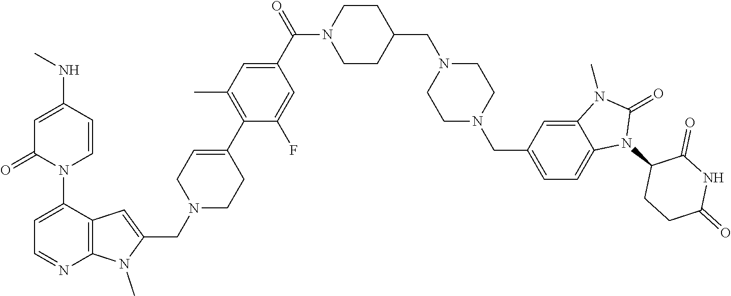 [SMILES: CNc1ccn(-c2ccnc3c2cc(CN2CC=C(c4c(C)cc(C(=O)N5CCC(CN6CCN(Cc7ccc8c(c7)n(C)c(=O)n8[C@@H]7CCC(=O)NC7=O)CC6)CC5)cc4F)CC2)n3C)c(=O)c1] |
| 3u163 | 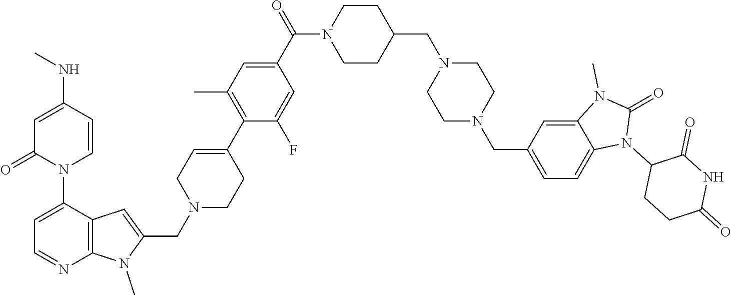 [SMILES: CNc1ccn(-c2ccnc3c2cc(CN2CC=C(c4c(C)cc(C(=O)N5CCC(CN6CCN(Cc7ccc8c(c7)n(C)c(=O)n8C7CCC(=O)NC7=O)CC6)CC5)cc4F)CC2)n3C)c(=O)c1] |
| 3u164 | 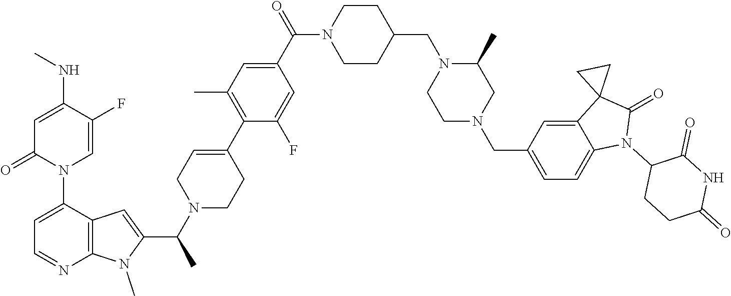 [SMILES: CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)cc1F] |
| 3u165 | 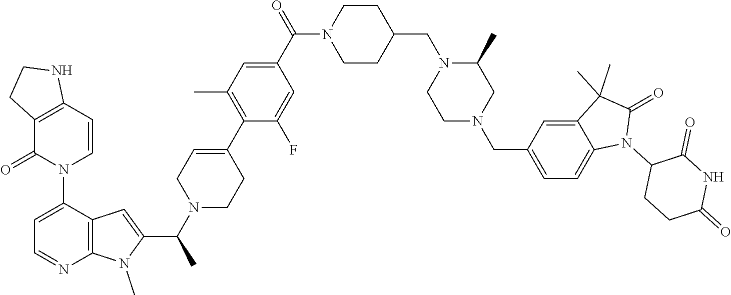 [SMILES: Cc1cc(C(=O)N2CCC(CN3CCN(Cc4ccc5c(c4)C(C)(C)C(=O)N5C4CCC(=O)NC4=O)C[C@@H]3C)CC2)cc(F)c1C1=CCN([C@@H](C)c2cc3c(-n4ccc5c(c4=O)CCN5)ccnc3n2C)CC1] |
| 3u166 | 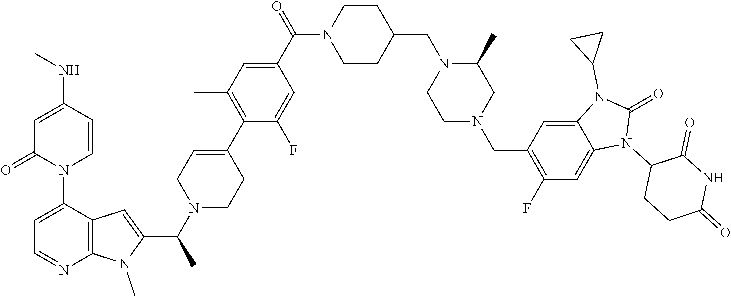 [SMILES: CNc1ccn(-c2ccnc3c2cc([C@H](C)N2CC=C(c4c(C)cc(C(=O)N5CCC(CN6CCN(Cc7cc8c(cc7F)n(C7CCC(=O)NC7=O)c(=O)n8C7CC7)C[C@@H]6C)CC5)cc4F)CC2)n3C)c(=O)c1] |
| 3u167 and 3u168 | 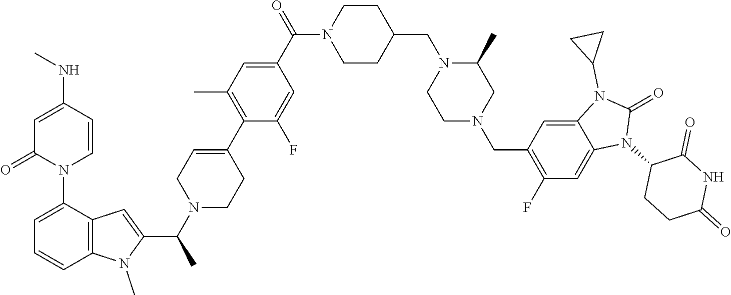 [SMILES: CNc1ccn(-c2cccc3c2cc([C@H](C)N2CC=C(c4c(C)cc(C(=O)N5CCC(CN6CCN(Cc7cc8c(cc7F)n([C@H]7CCC(=O)NC7=O)c(=O)n8C7CC7)C[C@@H]6C)CC5)cc4F)CC2)n3C)c(=O)c1] |
| | 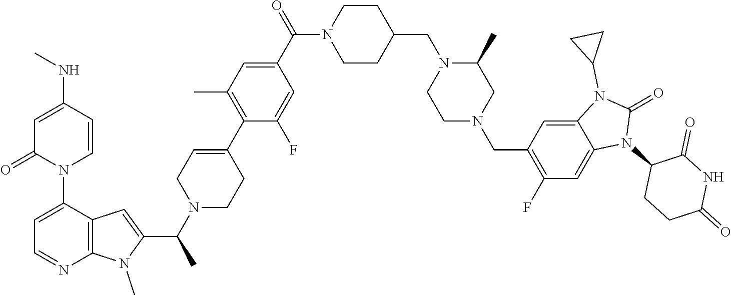 [SMILES: CNc1ccn(-c2ccnc3c2cc([C@H](C)N2CC=C(c4c(C)cc(C(=O)N5CCC(CN6CCN(Cc7cc8c(cc7F)n([C@@H]7CCC(=O)NC7=O)c(=O)n8C7CC7)C[C@@H]6C)CC5)cc4F)CC2)n3C)c(=O)c1] |
| 3u169 | 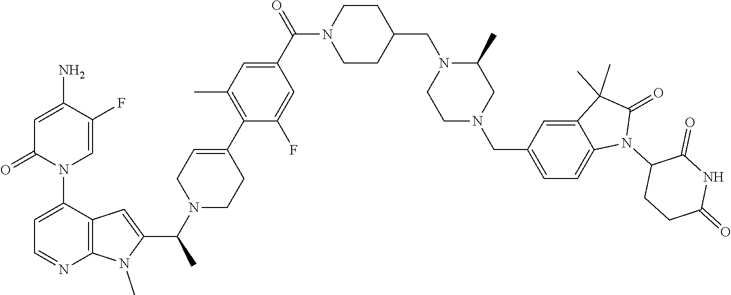 [SMILES: Cc1cc(C(=O)N2CCC(CN3CCN(Cc4ccc5c(c4)C(C)(C)C(=O)N5C4CCC(=O)NC4=O)C[C@@H]3C)CC2)cc(F)c1C1=CCN([C@@H](C)c2cc3c(-n4cc(F)c(N)cc4=O)ccnc3n2C)CC1] |
| 3u17 and 3u18 | 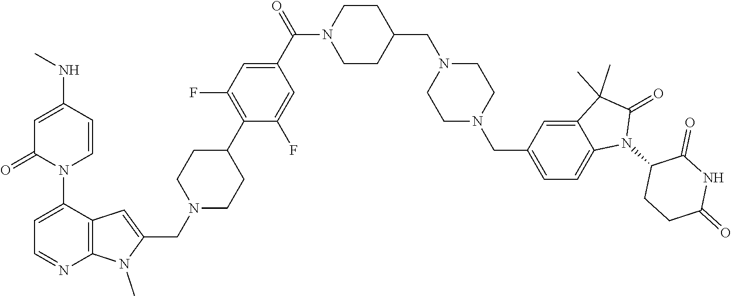 [SMILES: CNc1ccn(-c2ccnc3c2cc(CN2CCC(c4c(F)cc(C(=O)N5CCC(CN6CCN(Cc7ccc8c(c7)C(C)(C)C(=O)N8[C@H]7CCC(=O)NC7=O)CC6)CC5)cc4F)CC2)n3C)c(=O)c1] |
| | 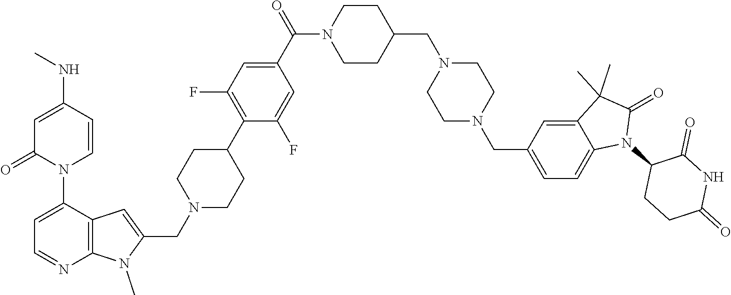 [SMILES: CNc1ccn(-c2ccnc3c2cc(CN2CCC(c4c(F)cc(C(=O)N5CCC(CN6CCN(Cc7ccc8c(c7)C(C)(C)C(=O)N8[C@@H]7CCC(=O)NC7=O)CC6)CC5)cc4F)CC2)n3C)c(=O)c1] |
| 3u170 and 3u171 | 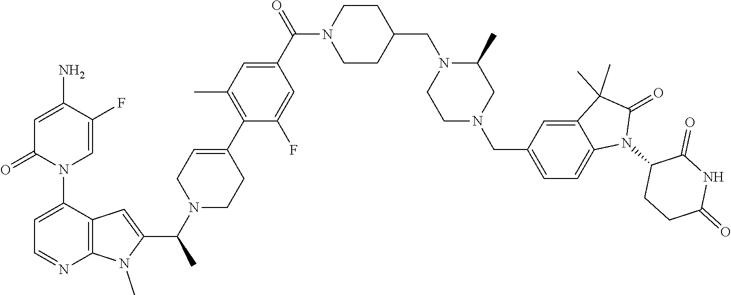 [SMILES: Cc1cc(C(=O)N2CCC(CN3CCN(Cc4ccc5c(c4)C(C)(C)C(=O)N5[C@H]4CCC(=O)NC4=O)C[C@@H]3C)CC2)cc(F)c1C1=CCN([C@@H](C)c2cc3c(-n4cc(F)c(N)cc4=O)ccnc3n2C)CC1] |
| | 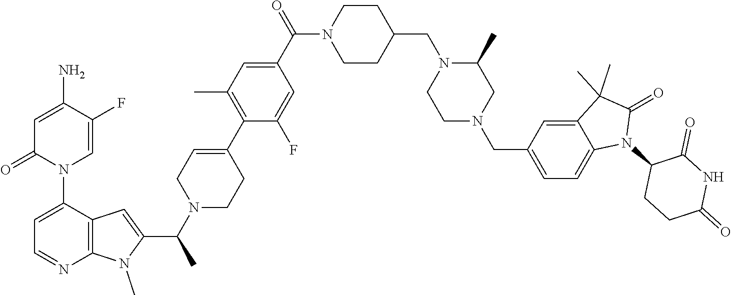 [SMILES: Cc1cc(C(=O)N2CCC(CN3CCN(Cc4ccc5c(c4)C(C)(C)C(=O)N5[C@@H]4CCC(=O)NC4=O)C[C@@H]3C)CC2)cc(F)c1C1=CCN([C@@H](C)c2cc3c(-n4cc(F)c(N)cc4=O)ccnc3n2C)CC1] |
| 3u172 | 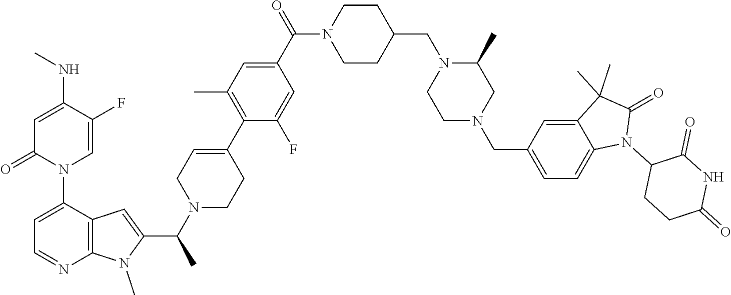 [SMILES: CNc1cc(=O)n(-c2ccnc3c2cc([C@H](C)N2CC=C(c4c(C)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)cc1F] |
| 3u173 and 3u174 | 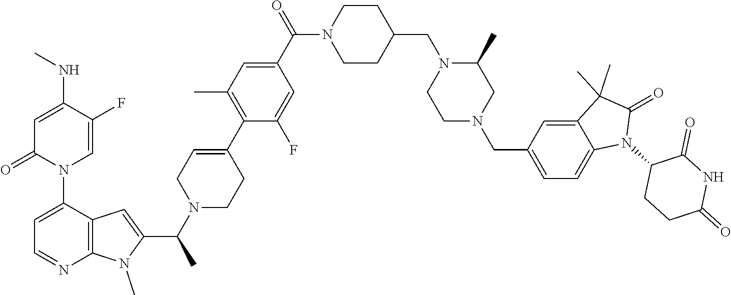 [SMILES: CNc1cc(=O)n(-c2ccnc3c2cc([C@H](C)N2CC=C(c4c(C)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)cc1F] |
| | 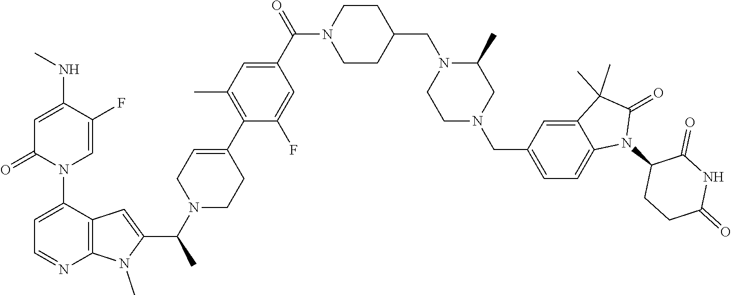 [SMILES: CNc1cc(=O)n(-c2ccnc3c2cc([C@H](C)N2CC=C(c4c(C)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)cc1F] |
| 3u175 | 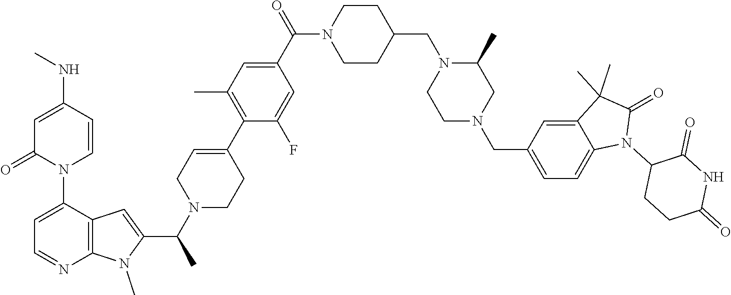 [SMILES: CNc1ccn(-c2ccnc3c2cc([C@H](C)N2CC=C(c4c(C)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)c(=O)c1] |
| 3u176 and 3u177 | 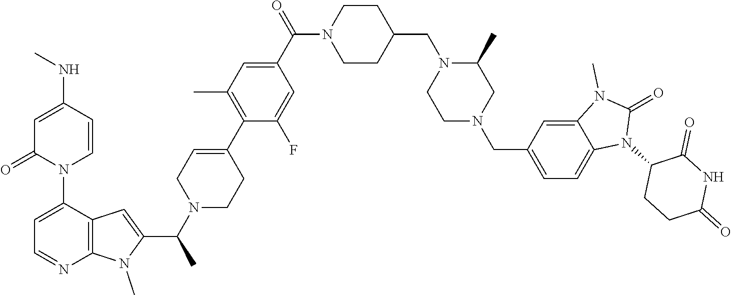 [SMILES: CNc1ccn(-c2ccnc3c2cc([C@H](C)N2CC=C(c4c(C)cc(C(=O)N5CCC(CN6CCN(Cc7ccc8c(c7)n(C)c(=O)n8[C@H]7CCC(=O)NC7=O)C[C@@H]6C)CC5)cc4F)CC2)n3C)c(=O)c1] |
| | 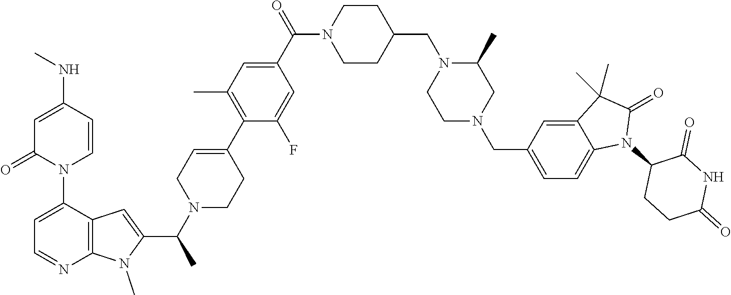 [SMILES: CNc1ccn(-c2ccnc3c2cc([C@H](C)N2CC=C(c4c(C)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)c(=O)c1] |
| 3u178 | 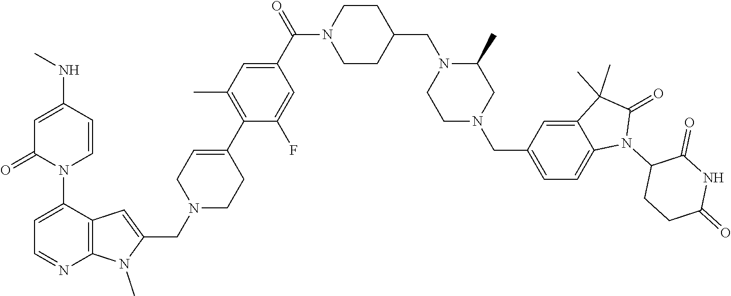 [SMILES: CNc1ccn(-c2ccnc3c2cc(CN2CC=C(c4c(C)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)c(=O)c1] |
| 3u179 and 3u180 | 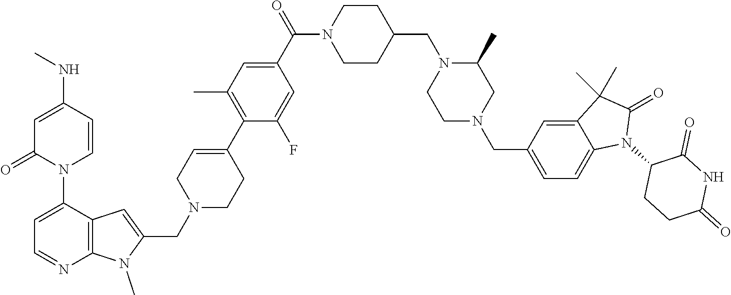 [SMILES: CNc1ccn(-c2ccnc3c2cc(CN2CC=C(c4c(C)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)c(=O)c1] |
| | 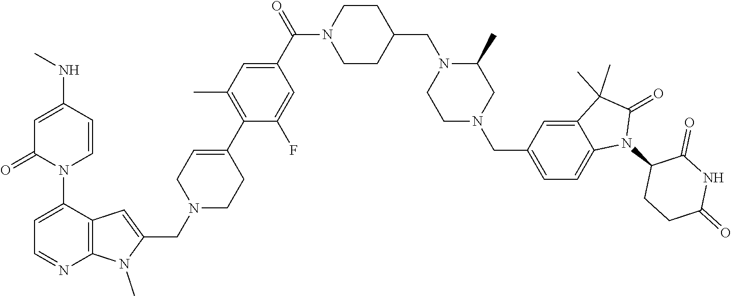 [SMILES: CNc1ccn(-c2ccnc3c2cc(CN2CC=C(c4c(C)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)c(=O)c1] |
| 3u181 | 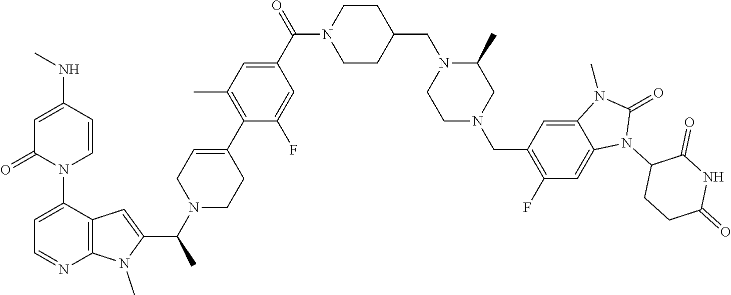 [SMILES: CNc1ccn(-c2ccnc3c2cc([C@H](C)N2CC=C(c4c(C)cc(C(=O)N5CCC(CN6CCN(Cc7cc8c(cc7F)n(C7CCC(=O)NC7=O)c(=O)n8C)C[C@@H]6C)CC5)cc4F)CC2)n3C)c(=O)c1] |
| 3u182 and 3u183 | 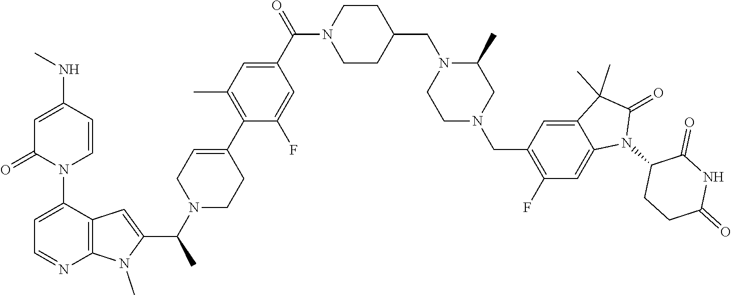 [SMILES: CNc1ccn(-c2ccnc3c2cc([C@H](C)N2CC=C(c4c(C)cc(C(=O)N5CCC(CN6CCN(Cc7cc8c(cc7F)N([C@H]7CCC(=O)NC7=O)C(=O)C8(C)C)C[C@@H]6C)CC5)cc4F)CC2)n3C)c(=O)c1] |
| | 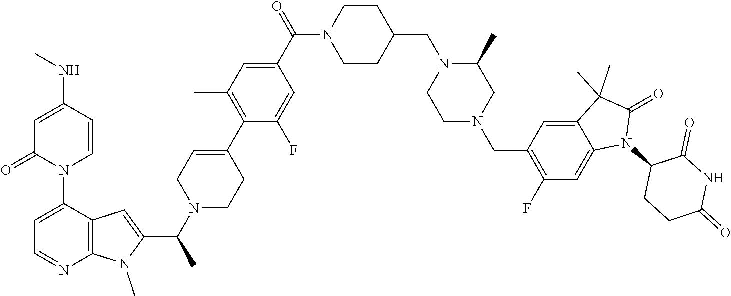 [SMILES: CNc1ccn(-c2ccnc3c2cc([C@H](C)N2CC=C(c4c(C)cc(C(=O)N5CCC(CN6CCN(Cc7cc8c(cc7F)N([C@@H]7CCC(=O)NC7=O)C(=O)C8(C)C)C[C@@H]6C)CC5)cc4F)CC2)n3C)c(=O)c1] |
| 3u184 | 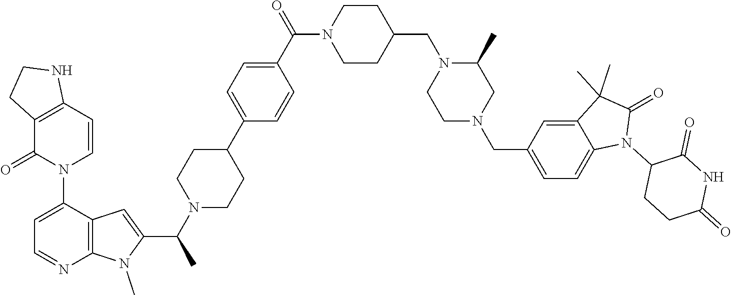 [SMILES: C[C@H]1CN(Cc2ccc3c(c2)C(C)(C)C(=O)N3C2CCC(=O)NC2=O)CCN1CC1CCN(C(=O)c2ccc(C3CCN([C@@H](C)c4cc5c(-n6ccc7c(c6=O)CCN7)ccnc5n4C)CC3)cc2)CC1] |
| 3u185 and 3u186 | 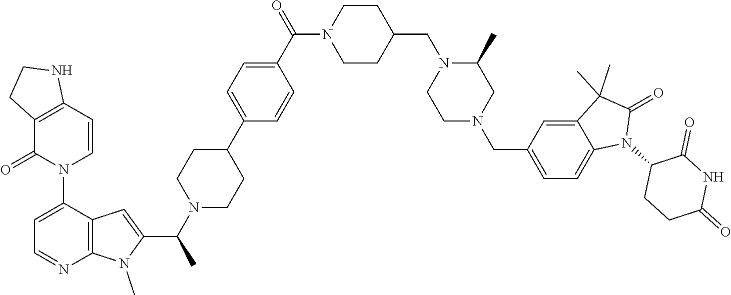 [SMILES: C[C@H]1CN(Cc2ccc3c(c2)C(C)(C)C(=O)N3[C@H]2CCC(=O)NC2=O)CCN1CC1CCN(C(=O)c2ccc(C3CCN([C@@H](C)c4cc5c(-n6ccc7c(c6=O)CCN7)ccnc5n4C)CC3)cc2)CC1] |
| | 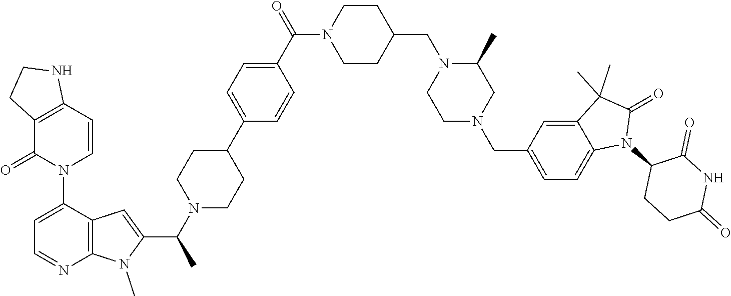 [SMILES: C[C@H]1CN(Cc2ccc3c(c2)C(C)(C)C(=O)N3[C@@H]2CCC(=O)NC2=O)CCN1CC1CCN(C(=O)c2ccc(C3CCN([C@@H](C)c4cc5c(-n6ccc7c(c6=O)CCN7)ccnc5n4C)CC3)cc2)CC1] |
| 3u187 | 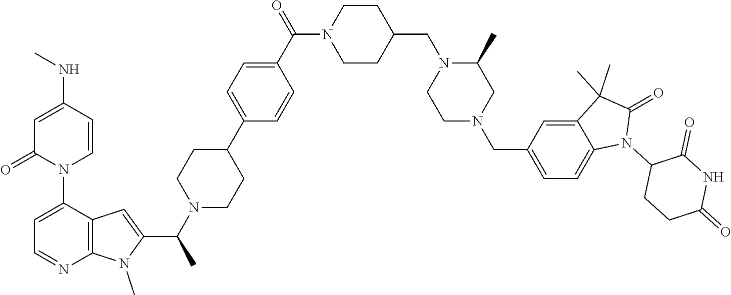 [SMILES: CNc1ccn(-c2ccnc3c2cc([C@H](C)N2CCC(c4ccc(C(=O)N5CCC(CN6CCN(Cc7ccc8c(c7)C(C)(C)C(=O)N8C7CCC(=O)NC7=O)C[C@@H]6C)CC5)cc4)CC2)n3C)c(=O)c1] |
| 3u188 and 3u189 | 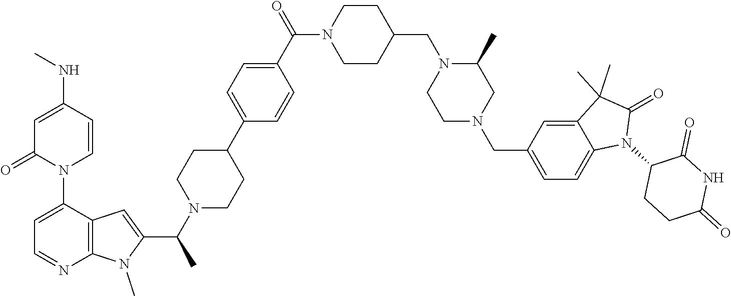 [SMILES: CNc1ccn(-c2ccnc3c2cc([C@H](C)N2CCC(c4ccc(C(=O)N5CCC(CN6CCN(Cc7ccc8c(c7)C(C)(C)C(=O)N8[C@H]7CCC(=O)NC7=O)C[C@@H]6C)CC5)cc4)CC2)n3C)c(=O)c1] |
| | 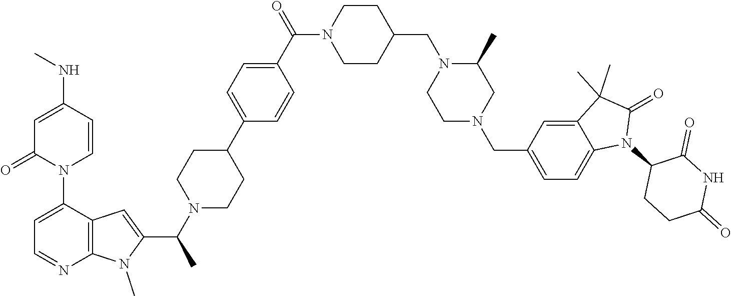 [SMILES: CNc1ccn(-c2ccnc3c2cc([C@H](C)N2CCC(c4ccc(C(=O)N5CCC(CN6CCN(Cc7ccc8c(c7)C(C)(C)C(=O)N8[C@@H]7CCC(=O)NC7=O)C[C@@H]6C)CC5)cc4)CC2)n3C)c(=O)c1] |
| 3u19 | 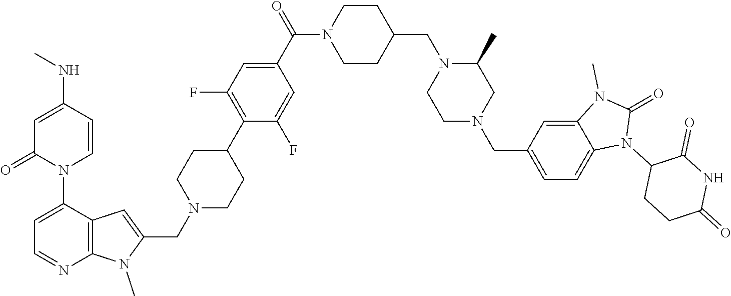 [SMILES: CNc1ccn(-c2ccnc3c2cc(CN2CCC(c4c(F)cc(C(=O)N5CCC(CN6CCN(Cc7ccc8c(c7)n(C)c(=O)n8C7CCC(=O)NC7=O)C[C@@H]6C)CC5)cc4F)CC2)n3C)c(=O)c1] |
| 3u190 | 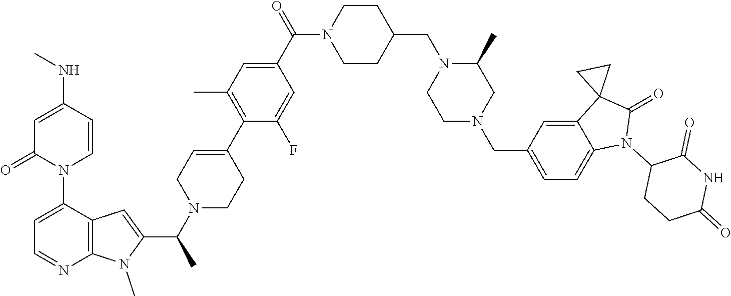 [SMILES: CNc1ccn(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)c(=O)c1] |
| 3u191 and 3u192 | 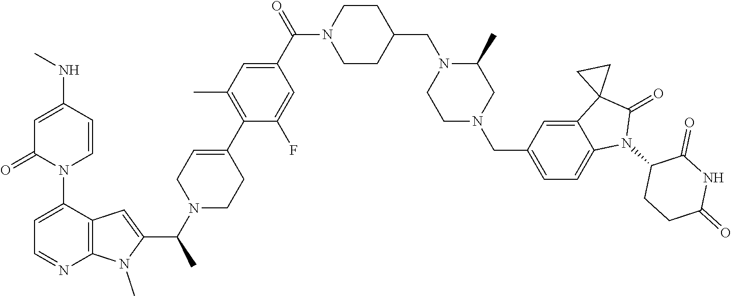 [SMILES: CNc1ccn(-c2ccnc3c2cc([C@H](C)N2CC=C(c4c(C)cc(C(=O)N5CCC(CN6CCN(Cc7ccc8c(c7)C7(CC7)C(=O)N8[C@H]7CCC(=O)NC7=O)C[C@@H]6C)CC5)cc4F)CC2)n3C)c(=O)c1] |
| | 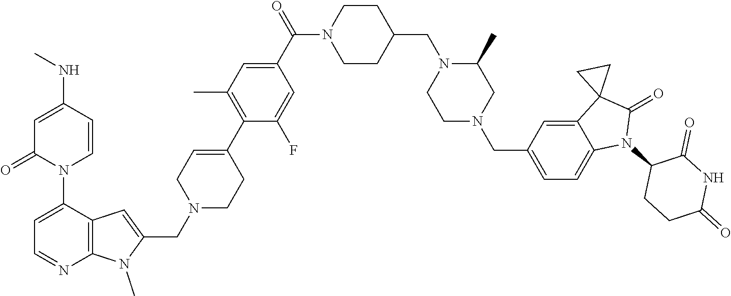 [SMILES: CNc1ccn(-c2ccnc3c2cc(CN2CC=C(c4c(C)cc(C(=O)N5CCC(CN6CCN(Cc7ccc8c(c7)C7(CC7)C(=O)N8[C@@H]7CCC(=O)NC7=O)C[C@@H]6C)CC5)cc4F)CC2)n3C)c(=O)c1] |
| 3u193 | 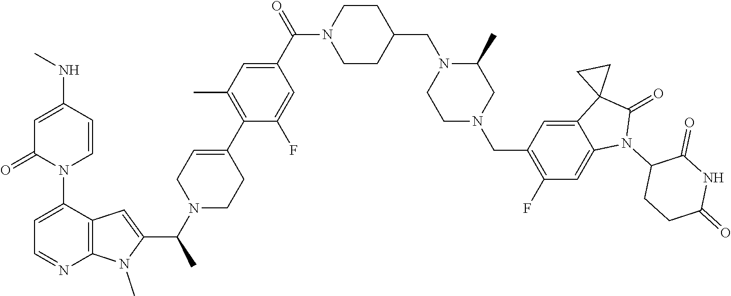 [SMILES: CNc1ccn(-c2ccnc3c2cc([C@H](C)N2CC=C(c4c(C)cc(C(=O)N5CCC(CN6CCN(Cc7cc8c(cc7F)N(C7CCC(=O)NC7=O)C(=O)C87CC7)C[C@@H]6C)CC5)cc4F)CC2)n3C)c(=O)c1] |
| 3u194 and 3u195 | 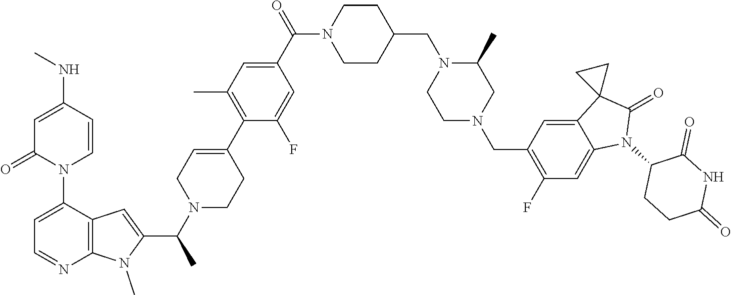 [SMILES: CNc1ccn(-c2ccnc3c2cc([C@H](C)N2CC=C(c4c(C)cc(C(=O)N5CCC(CN6CCN(Cc7cc8c(cc7F)N([C@H]7CCC(=O)NC7=O)C(=O)C87CC7)C[C@@H]6C)CC5)cc4F)CC2)n3C)c(=O)c1] |
| | 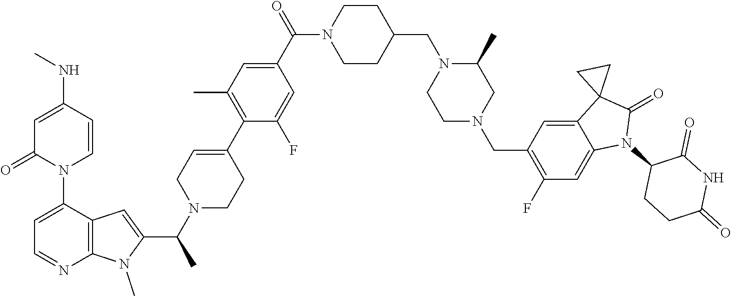 [SMILES: CNc1ccn(-c2ccnc3c2cc([C@H](C)N2CC=C(c4c(C)cc(C(=O)N5CCC(CN6CCN(Cc7cc8c(cc7F)N([C@@H]7CCC(=O)NC7=O)C(=O)C87CC7)C[C@@H]6C)CC5)cc4F)CC2)n3C)c(=O)c1] |
| 3u196 | 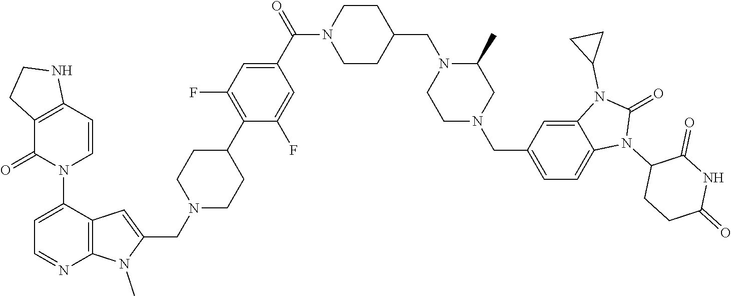 [SMILES: C[C@H]1CN(Cc2ccc3c(c2)n(C2CC2)c(=O)n3C2CCC(=O)NC2=O)CCN1CC1CCN(C(=O)c2cc(F)c(C3CCN(Cc4cc5c(-n6ccc7c(c6=O)CCN7)ccnc5n4C)CC3)c(F)c2)CC1] |
| 3u197 | 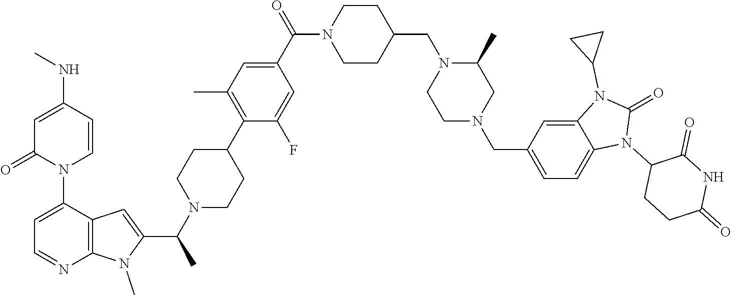 [SMILES: CNc1ccn(-c2ccnc3c2cc([C@H](C)N2CCC(c4c(C)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)c(=O)c1] |
| 3u198 and 3u199 | 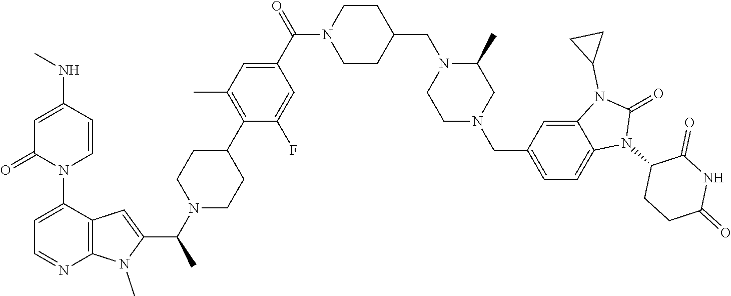 [SMILES: CNc1ccn(-c2ccnc3c2cc([C@H](C)N2CCC(c4c(C)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)c(=O)c1] |
| | 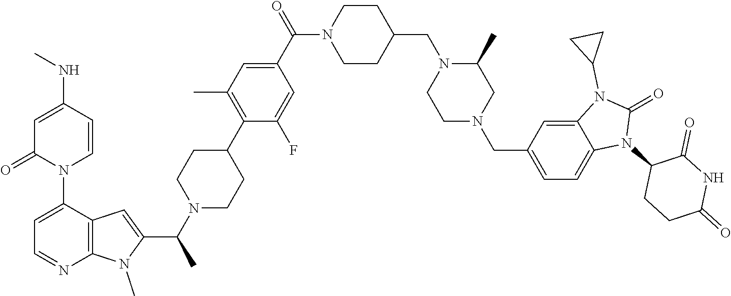 [SMILES: CNc1ccn(-c2ccnc3c2cc([C@H](C)N2CCC(c4c(C)cc(C(=O)N5CCC(CN6CCN(Cc7ccc8c(c7)n(C7CC7)c(=O)n8[C@@H]7CCC(=O)NC7=O)C[C@@H]6C)CC5)cc4F)CC2)n3C)c(=O)c1] |
| 3u2 and 3u3 | 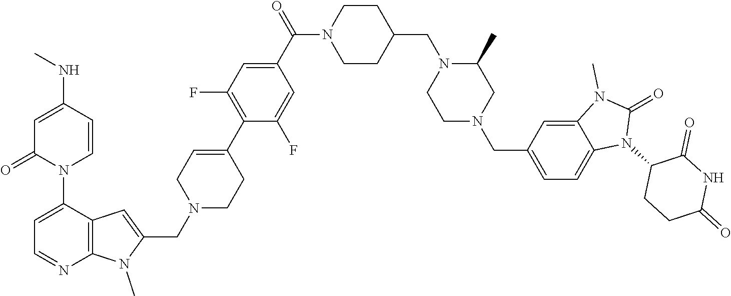 [SMILES: CNc1ccn(-c2ccnc3c2cc(CN2CC=C(c4c(F)cc(C(=O)N5CCC(CN6CCN(Cc7ccc8c(c7)n(C)c(=O)n8[C@H]7CCC(=O)NC7=O)C[C@@H]6C)CC5)cc4F)CC2)n3C)c(=O)c1] |
| | 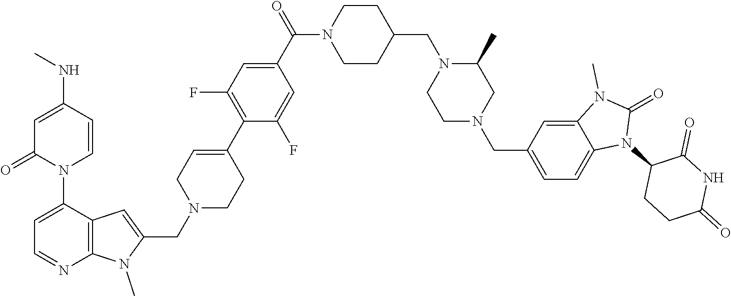 [SMILES: CNc1ccn(-c2ccnc3c2cc(CN2CC=C(c4c(F)cc(C(=O)N5CCC(CN6CCN(Cc7ccc8c(c7)n(C)c(=O)n8[C@@H]7CCC(=O)NC7=O)C[C@@H]6C)CC5)cc4F)CC2)n3C)c(=O)c1] |
| 3u20 and 3u21 | 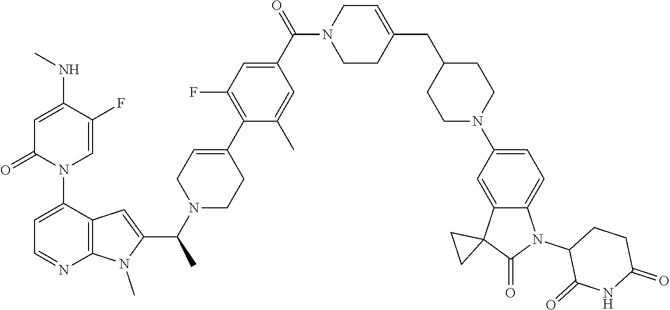 [SMILES: CNc1cc(=O)n(-c2ccnc3c2cc([C@H](C)N2CC=C(c4c(C)cc(C(=O)N5CC=C(CC6CCN(c7ccc8c(c7)C7(CC7)C(=O)N8C7CCC(=O)NC7=O)CC6)CC5)cc4F)CC2)n3C)cc1F] |
| | 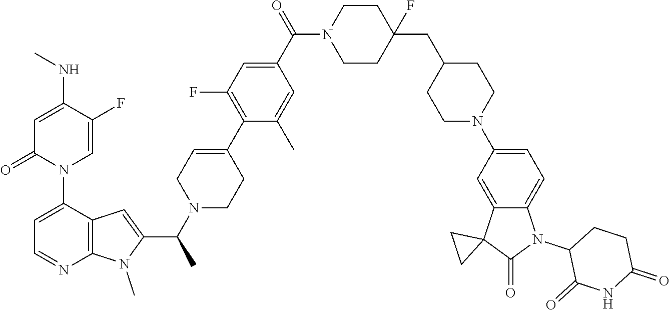 [SMILES: CNc1cc(=O)n(-c2ccnc3c2cc([C@H](C)N2CC=C(c4c(C)cc(C(=O)N5CCC(F)(CC6CCN(c7ccc8c(c7)C7(CC7)C(=O)N8C7CCC(=O)NC7=O)CC6)CC5)cc4F)CC2)n3C)cc1F] |
| 3u200 | 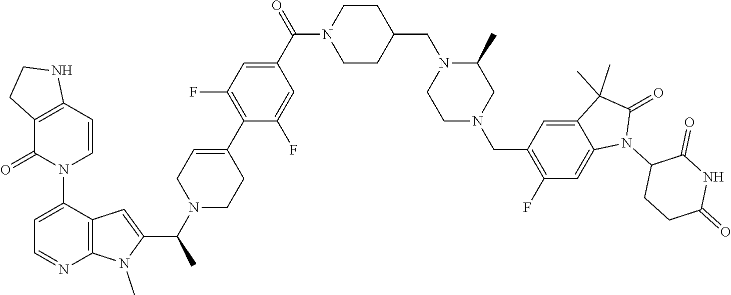 [SMILES: C[C@H]1CN(Cc2cc3c(cc2F)N(C2CCC(=O)NC2=O)C(=O)C3(C)C)CCN1CC1CCN(C(=O)c2cc(F)c(C3=CCN([C@@H](C)c4cc5c(-n6ccc7c(c6=O)CCN7)ccnc5n4C)CC3)c(F)c2)CC1] |
| 3u201 | 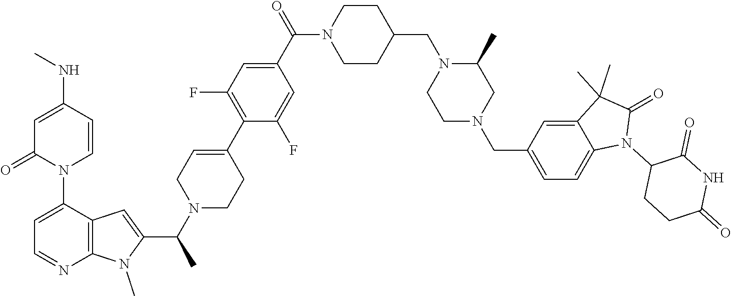 [SMILES: CNc1ccn(-c2ccnc3c2cc([C@H](C)N2CC=C(c4c(F)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)c(=O)c1] |
| 3u202 and 3u203 | 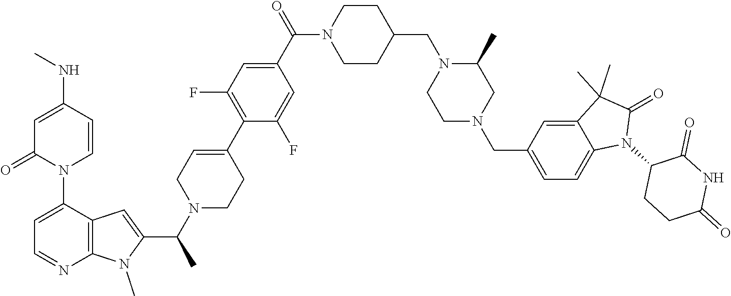 [SMILES: CNc1ccn(-c2ccnc3c2cc([C@H](C)N2CC=C(c4c(F)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)c(=O)c1] |
| | 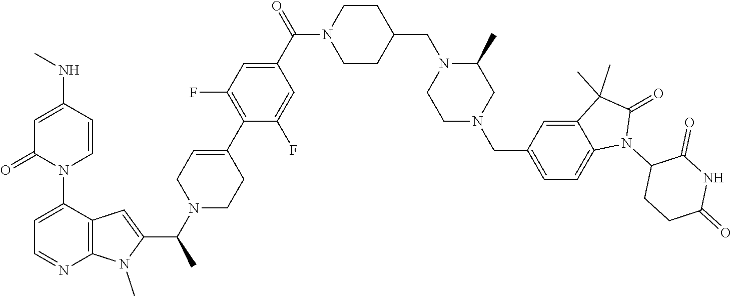 [SMILES: CNc1ccn(-c2ccnc3c2cc([C@H](C)N2CC=C(c4c(F)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)c(=O)c1] |
| 3u204 | 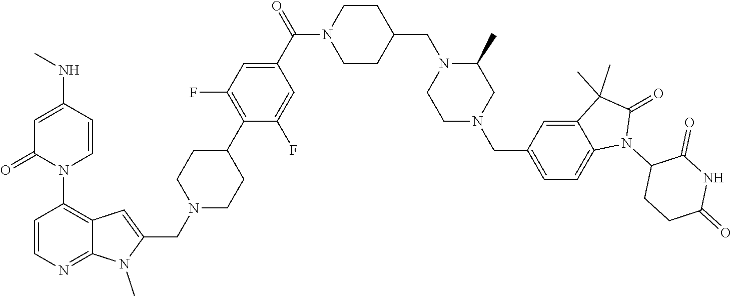 [SMILES: CNc1ccn(-c2ccnc3c2cc(CN2CCC(c4c(F)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)c(=O)c1] |
| 3u205 and 3u206 | 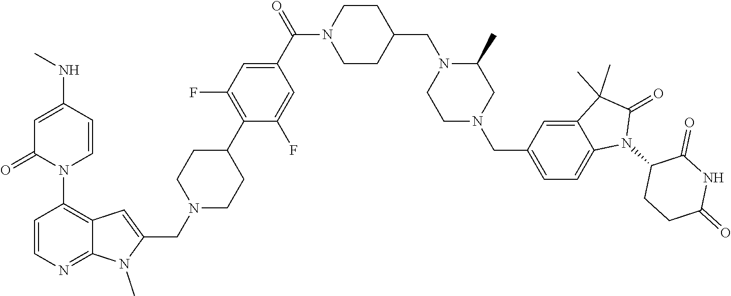 [SMILES: CNc1ccn(-c2ccnc3c2cc(CN2CCC(c4c(F)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)c(=O)c1] |
| | 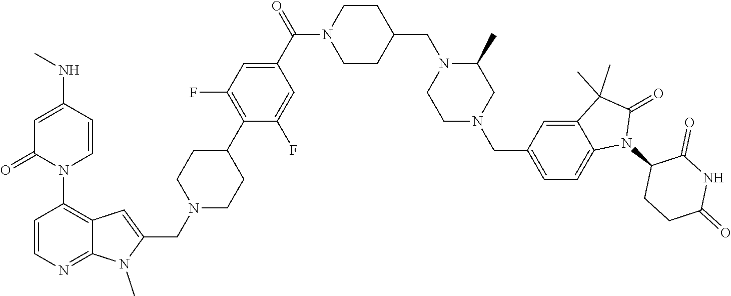 [SMILES: CNc1ccn(-c2ccnc3c2cc(CN2CCC(c4c(F)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)c(=O)c1] |
| 3u207 | 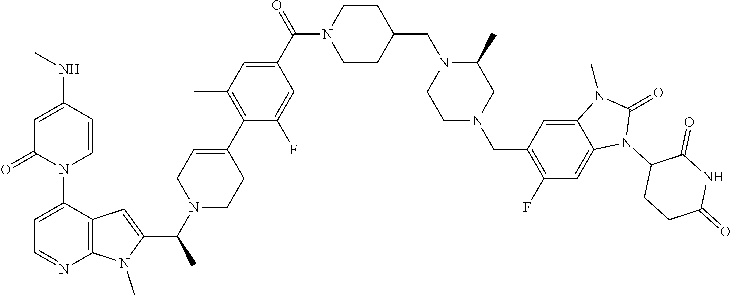 [SMILES: CNc1ccn(-c2ccnc3c2cc([C@H](C)N2CC=C(c4c(C)cc(C(=O)N5CCC(CN6CCN(Cc7cc8c(cc7F)n(C7CCC(=O)NC7=O)c(=O)n8C)C[C@@H]6C)CC5)cc4F)CC2)n3C)c(=O)c1] |
| 3u208 and 3u209 | 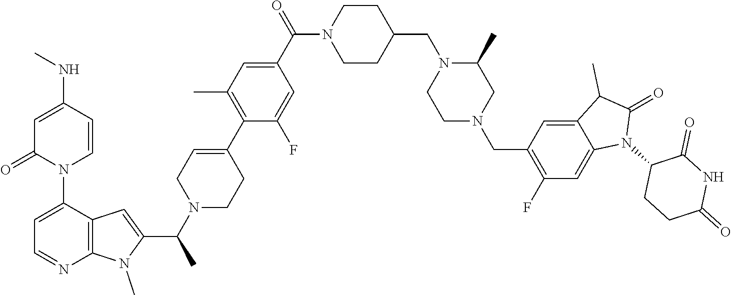 [SMILES: CNc1ccn(-c2ccnc3c2cc([C@H](C)N2CC=C(c4c(C)cc(C(=O)N5CCC(CN6CCN(Cc7cc8c(cc7F)N([C@H]7CCC(=O)NC7=O)C(=O)C8C)C[C@@H]6C)CC5)cc4F)CC2)n3C)c(=O)c1] |
| | 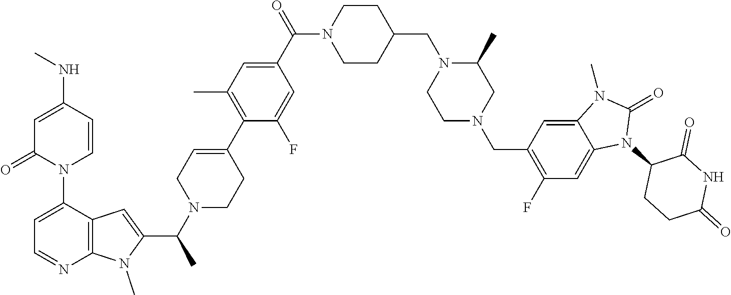 [SMILES: CNc1ccn(-c2ccnc3c2cc([C@H](C)N2CC=C(c4c(C)cc(C(=O)N5CCC(CN6CCN(Cc7cc8c(cc7F)n([C@@H]7CCC(=O)NC7=O)c(=O)n8C)C[C@@H]6C)CC5)cc4F)CC2)n3C)c(=O)c1] |
| 3u210 | 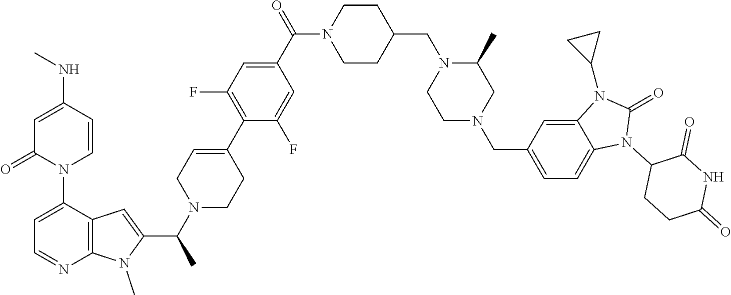 [SMILES: CNc1ccn(-c2ccnc3c2cc([C@H](C)N2CC=C(c4c(F)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)c(=O)c1] |
| 3u211 and 3u212 | 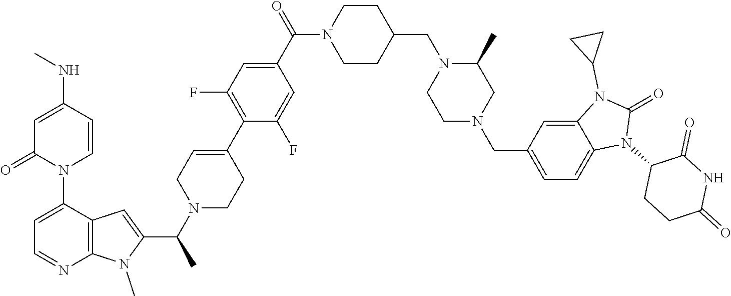 [SMILES: CNc1ccn(-c2ccnc3c2cc([C@H](C)N2CC=C(c4c(F)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)c(=O)c1] |
| | 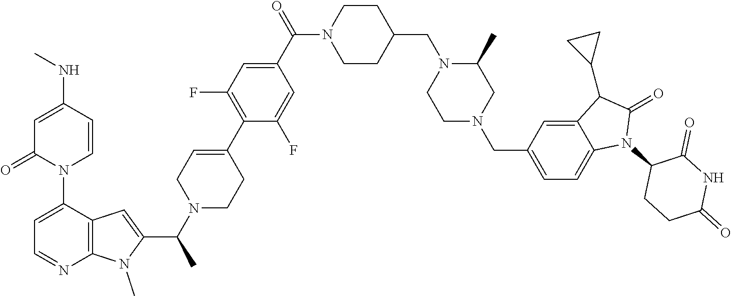 [SMILES: CNc1ccn(-c2ccnc3c2cc([C@H](C)N2CC=C(c4c(F)cc(C(=O)N5CCC(CN6CCN(Cc7ccc8c(c7)C(C7CC7)C(=O)N8[C@@H]7CCC(=O)NC7=O)C[C@@H]6C)CC5)cc4F)CC2)n3C)c(=O)c1] |
| 3u213 | 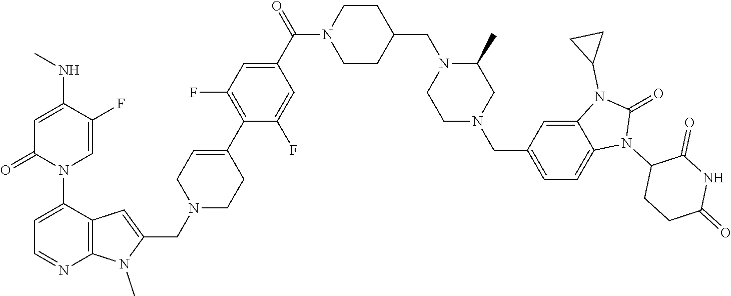 [SMILES: CNc1cc(=O)n(-c2ccnc3c2cc(CN2CC=C(c4c(F)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)cc1F] |
| 3u214 and 3u215 | 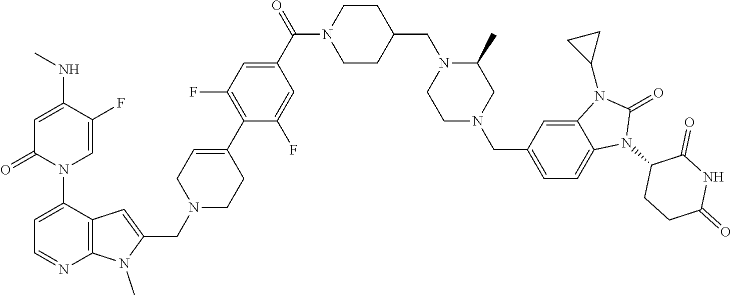 [SMILES: CNc1cc(=O)n(-c2ccnc3c2cc(CN2CC=C(c4c(F)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)cc1F] |
| | 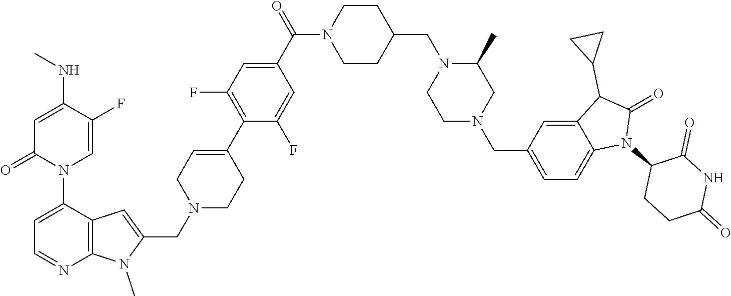 [SMILES: CNc1cc(=O)n(-c2ccnc3c2cc(CN2CC=C(c4c(F)cc(C(=O)N5CCC(CN6CCN(Cc7ccc8c(c7)C(C7CC7)C(=O)N8[C@@H]7CCC(=O)NC7=O)C[C@@H]6C)CC5)cc4F)CC2)n3C)cc1F] |
| 3u216 | 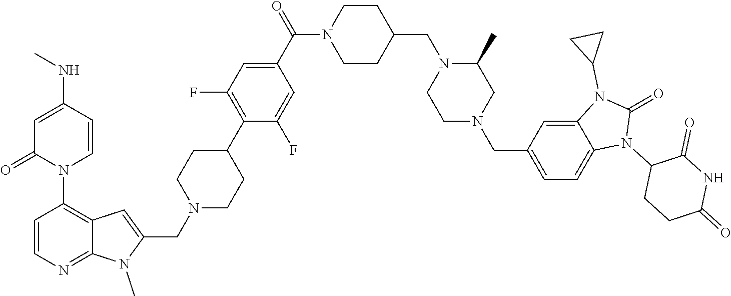 [SMILES: CNc1ccn(-c2ccnc3c2cc(CN2CCC(c4c(F)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)c(=O)c1] |
| 3u217 and 3u218 | 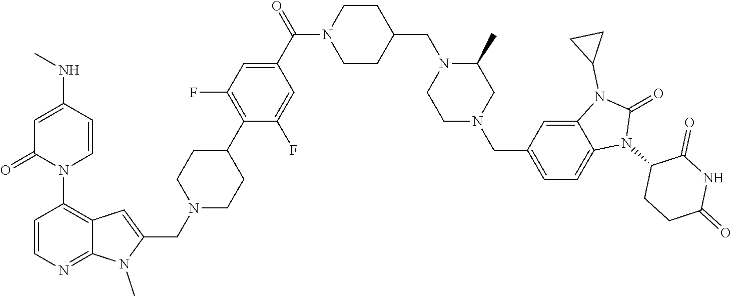 [SMILES: CNc1ccn(-c2ccnc3c2cc(CN2CCC(c4c(F)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)c(=O)c1] |
| | 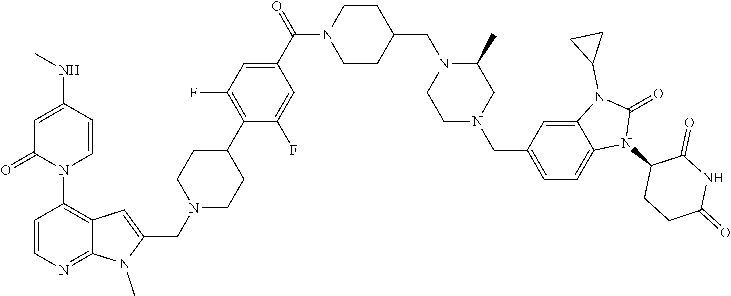 [SMILES: CNc1ccn(-c2ccnc3c2cc(CN2CCC(c4c(F)cc(C(=O)N5CCC(CN6CCN(Cc7ccc8c(c7)n(C7CC7)c(=O)n8[C@@H]7CCC(=O)NC7=O)C[C@@H]6C)CC5)cc4F)CC2)n3C)c(=O)c1] |
| 3u219 | 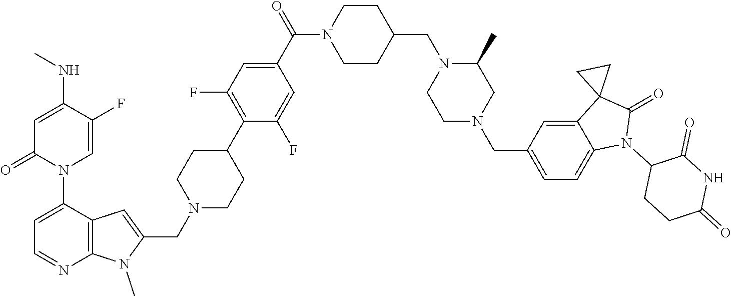 [SMILES: CNc1cc(=O)n(-c2ccnc3c2cc(CN2CCC(c4c(F)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)cc1F] |
| 3u22 | 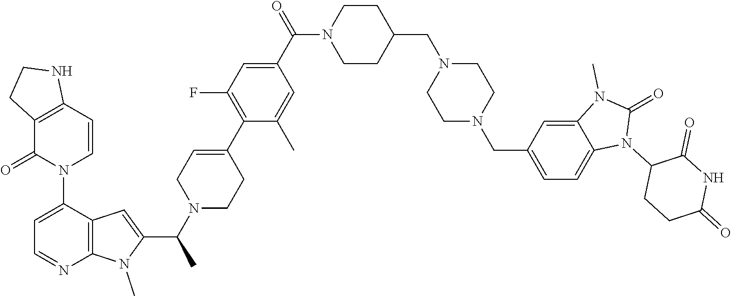 [SMILES: Cc1cc(C(=O)N2CCC(CN3CCN(Cc4ccc5c(c4)n(C)c(=O)n5C4CCC(=O)NC4=O)CC3)CC2)cc(F)c1C1=CCN([C@@H](C)c2cc3c(-n4ccc5c(c4=O)CCN5)ccnc3n2C)CC1] |
| 3u220 and 3u221 | 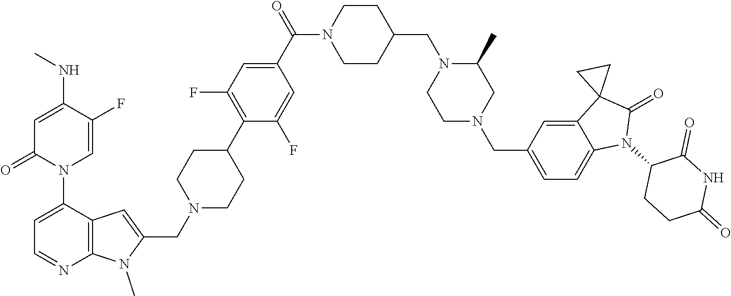 [SMILES: CNc1cc(=O)n(-c2ccnc3c2cc(CN2CCC(c4c(F)cc(C(=O)N5CCC(CN6CCN(Cc7ccc8c(c7)C7(CC7)C(=O)N8[C@H]7CCC(=O)NC7=O)C[C@@H]6C)CC5)cc4F)CC2)n3C)cc1F] |
| | 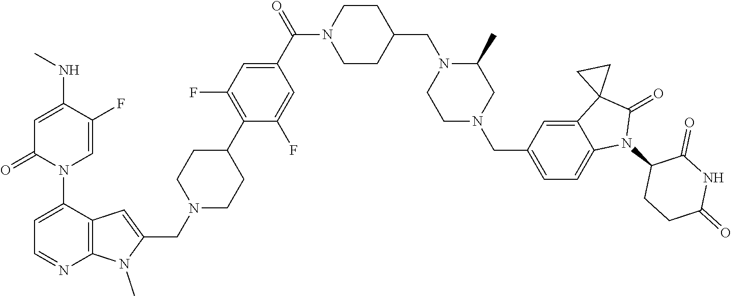 [SMILES: CNc1cc(=O)n(-c2ccnc3c2cc(CN2CCC(c4c(F)cc(C(=O)N5CCC(CN6CCN(Cc7ccc8c(c7)C7(CC7)C(=O)N8[C@@H]7CCC(=O)NC7=O)C[C@@H]6C)CC5)cc4F)CC2)n3C)cc1F] |
| 3u222 | 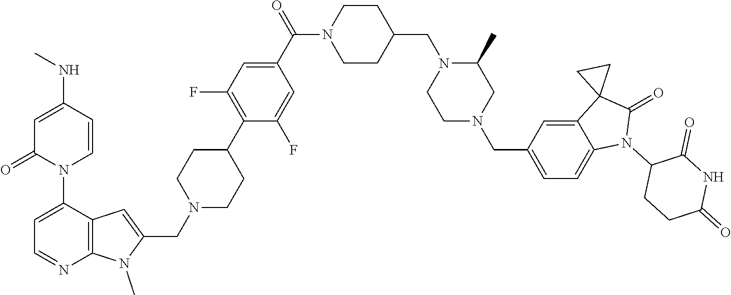 [SMILES: CNc1ccn(-c2ccnc3c2cc(CN2CCC(c4c(F)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)c(=O)c1] |
| 3u223 and 3u224 | 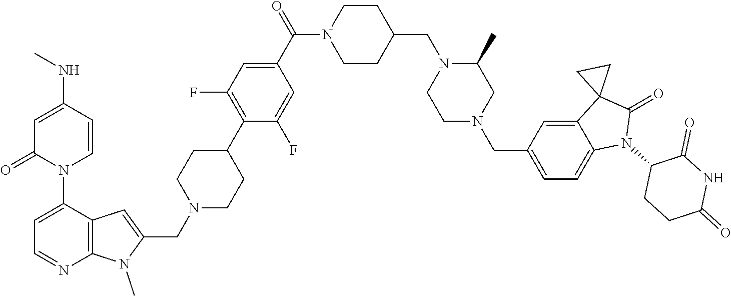 [SMILES: CNc1ccn(-c2ccnc3c2cc(CN2CCC(c4c(F)cc(C(=O)N5CCC(CN6CCN(Cc7ccc8c(c7)C7(CC7)C(=O)N8[C@H]7CCC(=O)NC7=O)C[C@@H]6C)CC5)cc4F)CC2)n3C)c(=O)c1] |
| | 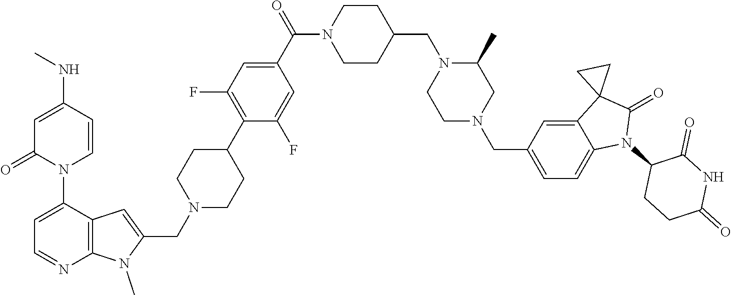 [SMILES: CNc1ccn(-c2ccnc3c2cc(CN2CCC(c4c(F)cc(C(=O)N5CCC(CN6CCN(Cc7ccc8c(c7)C7(CC7)C(=O)N8[C@@H]7CCC(=O)NC7=O)C[C@@H]6C)CC5)cc4F)CC2)n3C)c(=O)c1] |
| 3u225 | 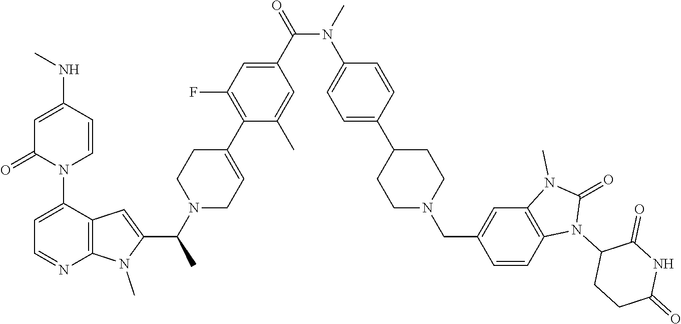 [SMILES: CNc1ccn(-c2ccnc3c2cc([C@H](C)N2CC=C(c4c(C)cc(C(=O)N(C)c5ccc(C6CCN(Cc7ccc8c(c7)n(C)c(=O)n8C7CCC(=O)NC7=O)CC6)cc5)cc4F)CC2)n3C)c(=O)c1] |
| 3u226 and 3u227 | 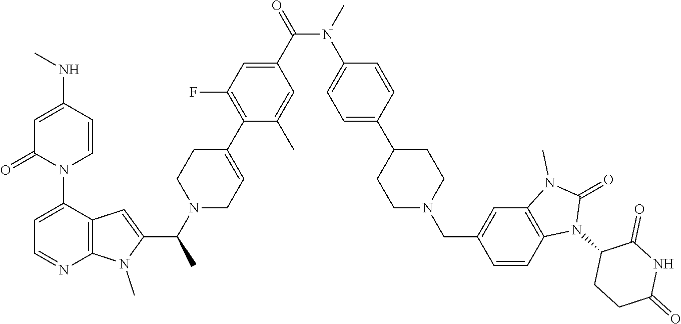 [SMILES: CNc1ccn(-c2ccnc3c2cc([C@H](C)N2CC=C(c4c(C)cc(C(=O)N(C)c5ccc(C6CCN(Cc7ccc8c(c7)n(C)c(=O)n8[C@H]7CCC(=O)NC7=O)CC6)cc5)cc4F)CC2)n3C)c(=O)c1] |
| | 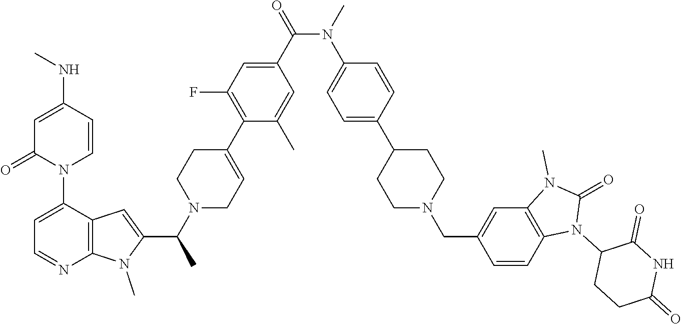 [SMILES: CNc1ccn(-c2ccnc3c2cc([C@H](C)N2CC=C(c4c(C)cc(C(=O)N(C)c5ccc(C6CCN(Cc7ccc8c(c7)n(C)c(=O)n8C7CCC(=O)NC7=O)CC6)cc5)cc4F)CC2)n3C)c(=O)c1] |
| 3u228 | 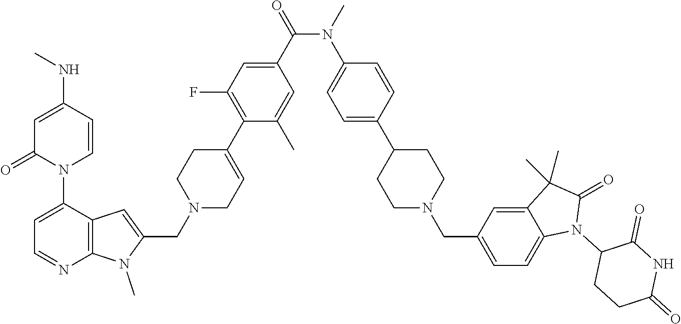 [SMILES: CNc1ccn(-c2ccnc3c2cc(CN2CC=C(c4c(C)cc(C(=O)N(C)c5ccc(C6CCN(Cc7ccc8c(c7)C(C)(C)C(=O)N8C7CCC(=O)NC7=O)CC6)cc5)cc4F)CC2)n3C)c(=O)c1] |
| 3u229 and 3u230 | 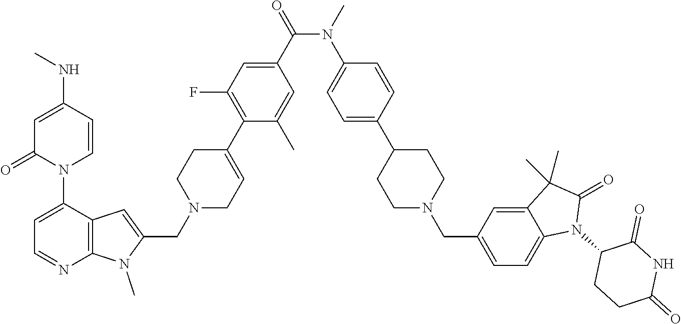 [SMILES: CNc1ccn(-c2ccnc3c2cc(CN2CC=C(c4c(C)cc(C(=O)N(C)c5ccc(C6CCN(Cc7ccc8c(c7)C(C)(C)C(=O)N8[C@H]7CCC(=O)NC7=O)CC6)cc5)cc4F)CC2)n3C)c(=O)c1] |
| | 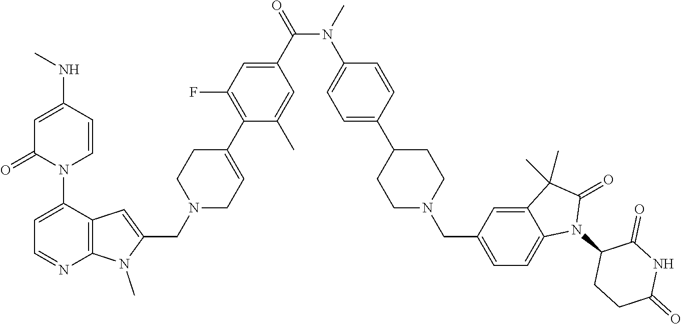 [SMILES: CNc1ccn(-c2ccnc3c2cc(CN2CC=C(c4c(C)cc(C(=O)N(C)c5ccc(C6CCN(Cc7ccc8c(c7)C(C)(C)C(=O)N8[C@@H]7CCC(=O)NC7=O)CC6)cc5)cc4F)CC2)n3C)c(=O)c1] |
| 3u23 | 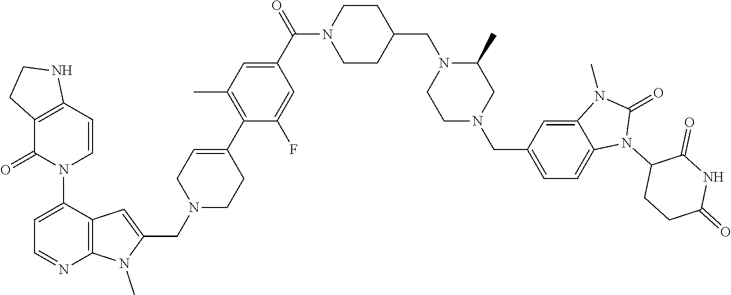 [SMILES: Cc1cc(C(=O)N2CCC(CN3CCN(Cc4ccc5c(c4)n(C)c(=O)n5C4CCC(=O)NC4=O)C[C@@H]3C)CC2)cc(F)c1C1=CCN(Cc2cc3c(-n4ccc5c(c4=O)CCN5)ccnc3n2C)CC1] |
| 3u231 | 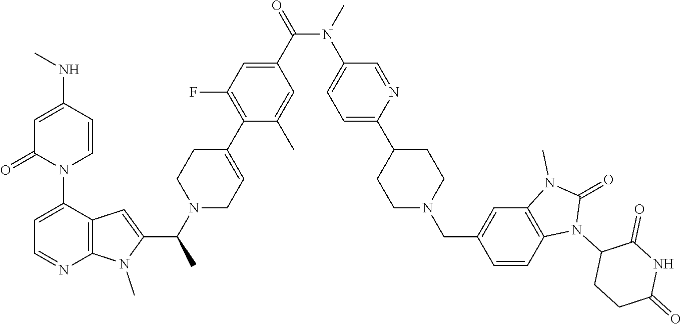 [SMILES: CNc1ccn(-c2ccnc3c2cc([C@H](C)N2CC=C(c4c(C)cc(C(=O)N(C)c5ccc(C6CCN(Cc7ccc8c(c7)n(C)c(=O)n8C7CCC(=O)NC7=O)CC6)nc5)cc4F)CC2)n3C)c(=O)c1] |
| 3u232 | 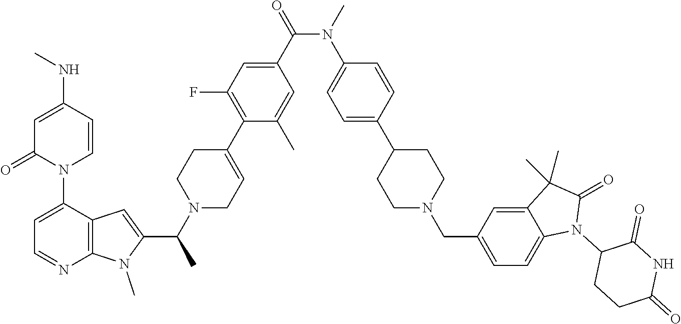 [SMILES: CNc1ccn(-c2ccnc3c2cc([C@H](C)N2CC=C(c4c(C)cc(C(=O)N(C)c5ccc(C6CCN(Cc7ccc8c(c7)C(C)(C)C(=O)N8C7CCC(=O)NC7=O)CC6)cc5)cc4F)CC2)n3C)c(=O)c1] |
| 3u233 | 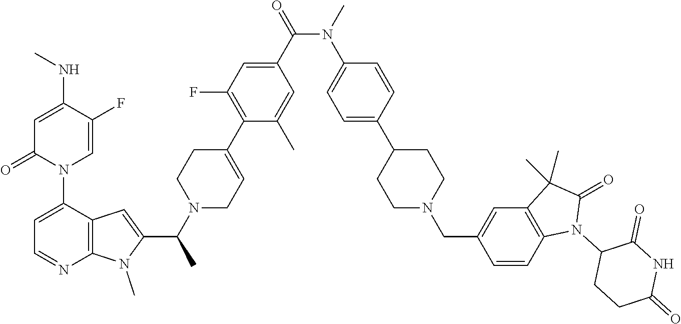 [SMILES: CNc1cc(=O)n(-c2ccnc3c2cc([C@H](C)N2CC=C(c4c(C)cc(C(=O)N(C)c5ccc(C6CCN(Cc7ccc8c(c7)C(C)(C)C(=O)N8C7CCC(=O)NC7=O)CC6)cc5)cc4F)CC2)n3C)cc1F] |
| 3u234 and 3u235 | 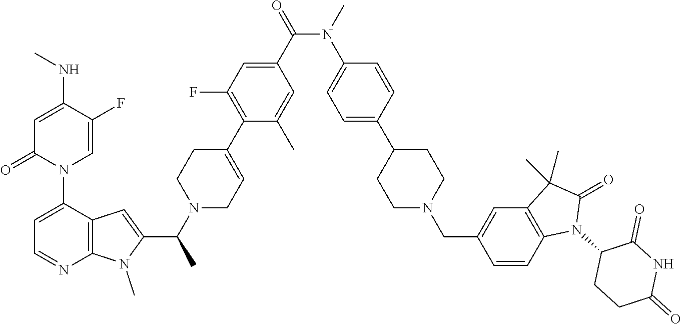 [SMILES: CNc1cc(=O)n(-c2ccnc3c2cc([C@H](C)N2CC=C(c4c(C)cc(C(=O)N(C)c5ccc(C6CCN(Cc7ccc8c(c7)C(C)(C)C(=O)N8[C@H]7CCC(=O)NC7=O)CC6)cc5)cc4F)CC2)n3C)cc1F] |
| | 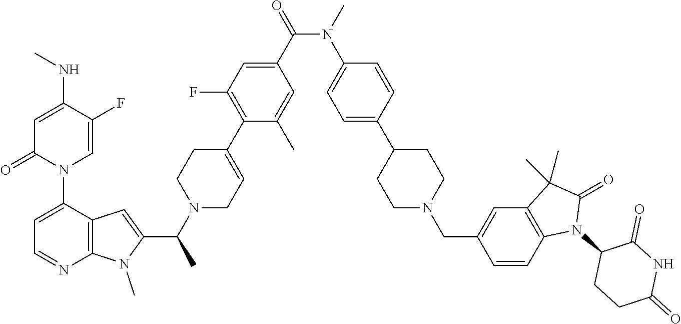 [SMILES: CNc1cc(=O)n(-c2ccnc3c2cc([C@H](C)N2CC=C(c4c(C)cc(C(=O)N(C)c5ccc(C6CCN(Cc7ccc8c(c7)C(C)(C)C(=O)N8[C@@H]7CCC(=O)NC7=O)CC6)cc5)cc4F)CC2)n3C)cc1F] |
| 3u236 | 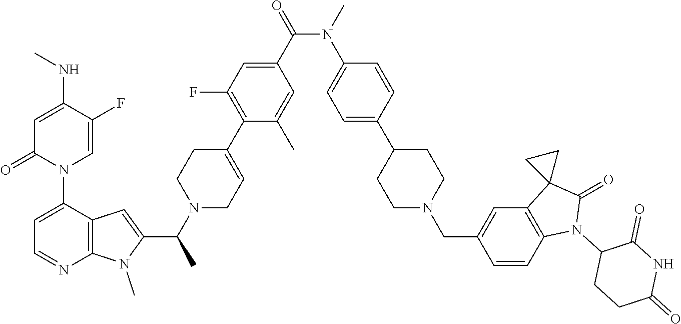 [SMILES: CNc1cc(=O)n(-c2ccnc3c2cc([C@H](C)N2CC=C(c4c(C)cc(C(=O)N(C)c5ccc(C6CCN(Cc7ccc8c(c7)C7(CC7)C(=O)N8C7CCC(=O)NC7=O)CC6)cc5)cc4F)CC2)n3C)cc1F] |
| 3u237 | 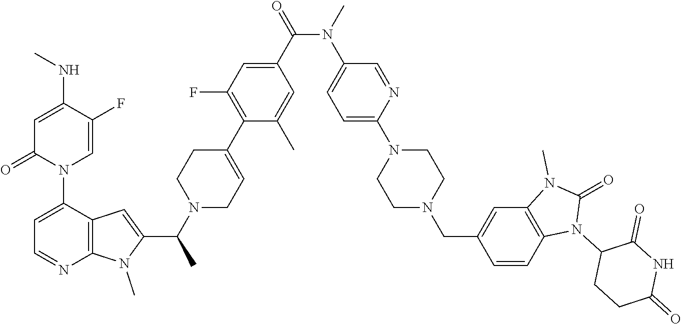 [SMILES: CNc1cc(=O)n(-c2ccnc3c2cc([C@H](C)N2CC=C(c4c(C)cc(C(=O)N(C)c5ccc(N6CCN(Cc7ccc8c(c7)n(C)c(=O)n8C7CCC(=O)NC7=O)CC6)nc5)cc4F)CC2)n3C)cc1F] |
| 3u238 | 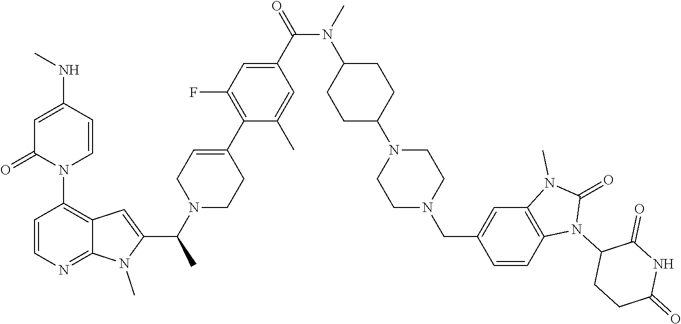 [SMILES: CNc1ccn(-c2ccnc3c2cc([C@H](C)N2CC=C(c4c(C)cc(C(=O)N(C)C5CCC(N6CCN(Cc7ccc8c(c7)n(C)c(=O)n8C7CCC(=O)NC7=O)CC6)CC5)cc4F)CC2)n3C)c(=O)c1] |
| 3u239 | 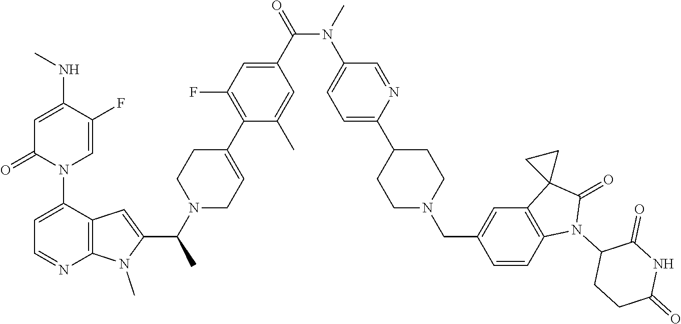 [SMILES: CNc1cc(=O)n(-c2ccnc3c2cc([C@H](C)N2CC=C(c4c(C)cc(C(=O)N(C)c5ccc(C6CCN(Cc7ccc8c(c7)C7(CC7)C(=O)N8C7CCC(=O)NC7=O)CC6)nc5)cc4F)CC2)n3C)cc1F] |
| 3u24 and 3u25 | 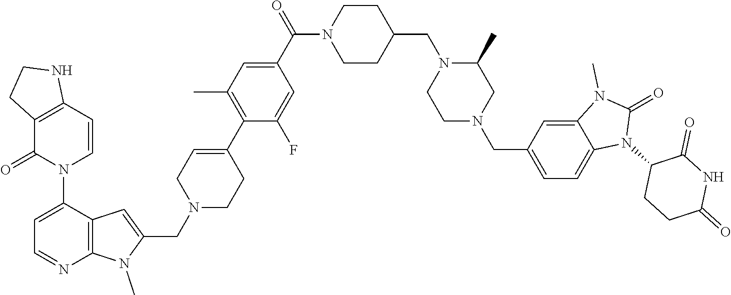 [SMILES: Cc1cc(C(=O)N2CCC(CN3CCN(Cc4ccc5c(c4)n(C)c(=O)n5[C@H]4CCC(=O)NC4=O)C[C@@H]3C)CC2)cc(F)c1C1=CCN(Cc2cc3c(-n4ccc5c(c4=O)CCN5)ccnc3n2C)CC1] |
| | 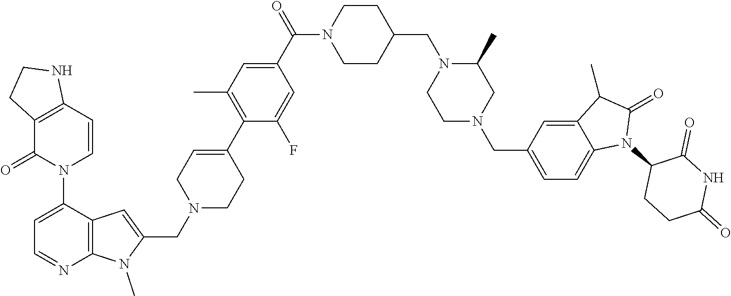 [SMILES: Cc1cc(C(=O)N2CCC(CN3CCN(Cc4ccc5c(c4)C(C)C(=O)N5[C@@H]4CCC(=O)NC4=O)C[C@@H]3C)CC2)cc(F)c1C1=CCN(Cc2cc3c(-n4ccc5c(c4=O)CCN5)ccnc3n2C)CC1] |
| 3u240 | 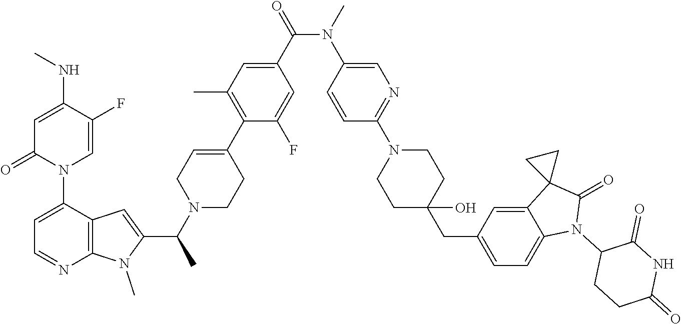 [SMILES: CNc1cc(=O)n(-c2ccnc3c2cc([C@H](C)N2CC=C(c4c(C)cc(C(=O)N(C)c5ccc(N6CCC(O)(Cc7ccc8c(c7)C7(CC7)C(=O)N8C7CCC(=O)NC7=O)CC6)nc5)cc4F)CC2)n3C)cc1F] |
| 3u241 | 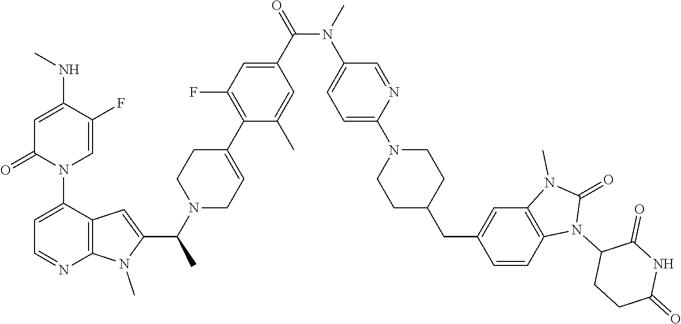 [SMILES: CNc1cc(=O)n(-c2ccnc3c2cc([C@H](C)N2CC=C(c4c(C)cc(C(=O)N(C)c5ccc(N6CCC(Cc7ccc8c(c7)n(C)c(=O)n8C7CCC(=O)NC7=O)CC6)nc5)cc4F)CC2)n3C)cc1F] |
| 3u242 and 3u243 | 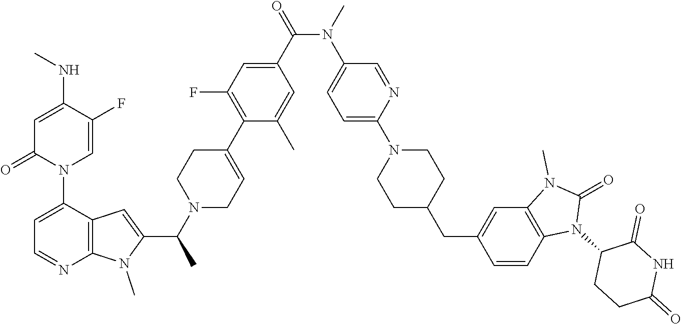 [SMILES: CNc1cc(=O)n(-c2ccnc3c2cc([C@H](C)N2CC=C(c4c(C)cc(C(=O)N(C)c5ccc(N6CCC(Cc7ccc8c(c7)n(C)c(=O)n8[C@H]7CCC(=O)NC7=O)CC6)nc5)cc4F)CC2)n3C)cc1F] |
| | 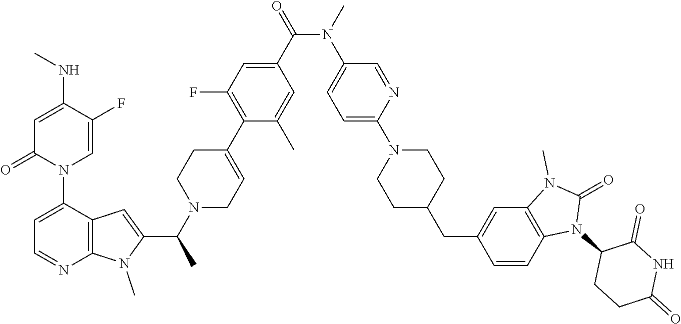 [SMILES: CNc1cc(=O)n(-c2ccnc3c2cc([C@H](C)N2CC=C(c4c(C)cc(C(=O)N(C)c5ccc(N6CCC(Cc7ccc8c(c7)n(C)c(=O)n8[C@@H]7CCC(=O)NC7=O)CC6)nc5)cc4F)CC2)n3C)cc1F] |
| 3u244 | 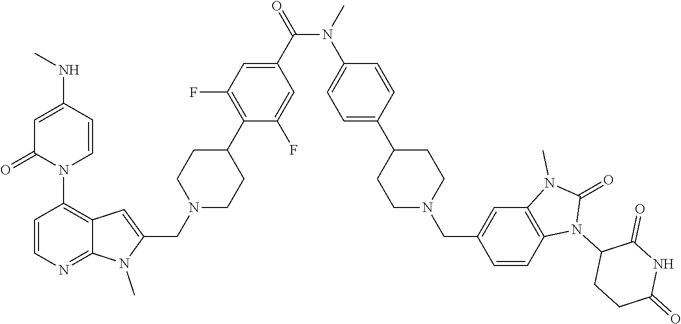 [SMILES: CNc1ccn(-c2ccnc3c2cc(CN2CCC(c4c(F)cc(C(=O)N(C)c5ccc(C6CCN(Cc7ccc8c(c7)n(C)c(=O)n8C7CCC(=O)NC7=O)CC6)cc5)cc4F)CC2)n3C)c(=O)c1] |
| 3u245 | 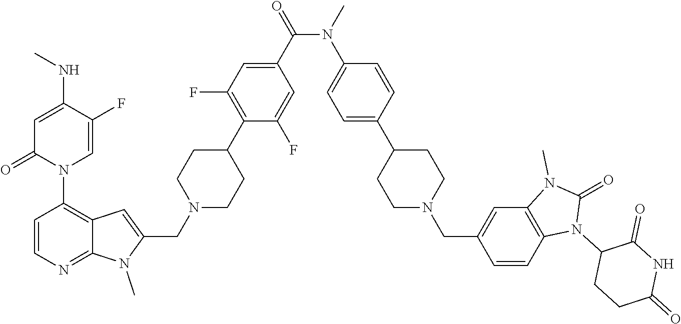 [SMILES: CNc1cc(=O)n(-c2ccnc3c2cc(CN2CCC(c4c(F)cc(C(=O)N(C)c5ccc(C6CCN(Cc7ccc8c(c7)n(C)c(=O)n8C7CCC(=O)NC7=O)CC6)cc5)cc4F)CC2)n3C)cc1F] |
| 3u246 | 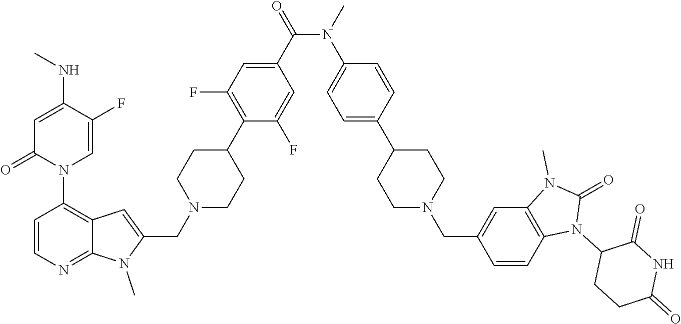 [SMILES: CNc1cc(=O)n(-c2ccnc3c2cc(CN2CCC(c4c(F)cc(C(=O)N(C)c5ccc(C6CCN(Cc7ccc8c(c7)n(C)c(=O)n8C7CCC(=O)NC7=O)CC6)cc5)cc4F)CC2)n3C)cc1F] |
| 3u247 | 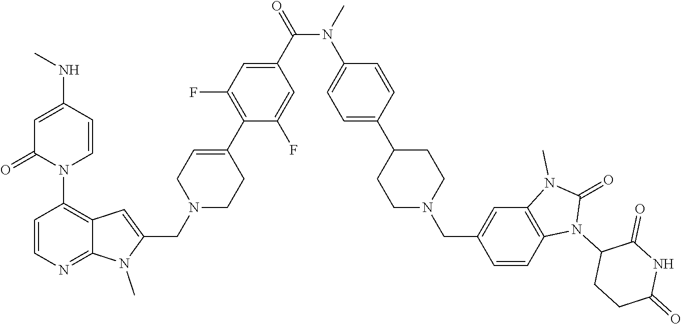 [SMILES: CNc1ccn(-c2ccnc3c2cc(CN2CC=C(c4c(F)cc(C(=O)N(C)c5ccc(C6CCN(Cc7ccc8c(c7)n(C)c(=O)n8C7CCC(=O)NC7=O)CC6)cc5)cc4F)CC2)n3C)c(=O)c1] |
| 3u248 | 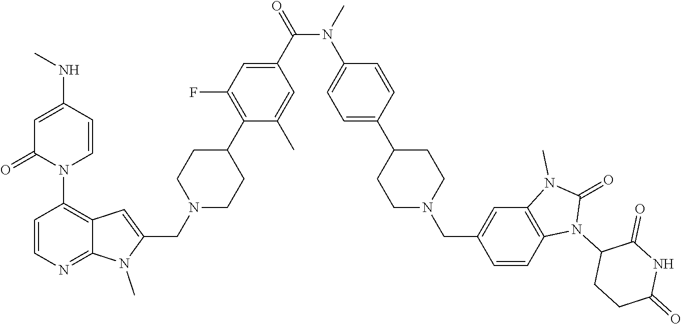 [SMILES: CNc1ccn(-c2ccnc3c2cc(CN2CCC(c4c(C)cc(C(=O)N(C)c5ccc(C6CCN(Cc7ccc8c(c7)n(C)c(=O)n8C7CCC(=O)NC7=O)CC6)cc5)cc4F)CC2)n3C)c(=O)c1] |
| 3u249 | 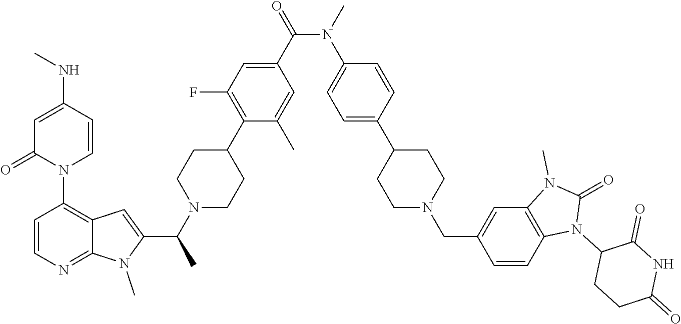 [SMILES: CNc1ccn(-c2ccnc3c2cc([C@H](C)N2CCC(c4c(C)cc(C(=O)N(C)c5ccc(C6CCN(Cc7ccc8c(c7)n(C)c(=O)n8C7CCC(=O)NC7=O)CC6)cc5)cc4F)CC2)n3C)c(=O)c1] |
| 3u250 | 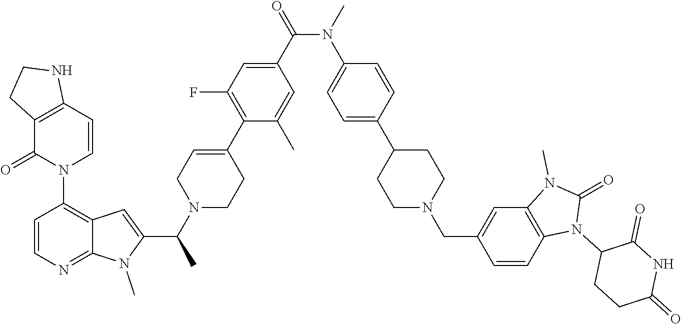 [SMILES: Cc1cc(C(=O)N(C)c2ccc(C3CCN(Cc4ccc5c(c4)n(C)c(=O)n5C4CCC(=O)NC4=O)CC3)cc2)cc(F)c1C1=CCN([C@@H](C)c2cc3c(-n4ccc5c(c4=O)CCN5)ccnc3n2C)CC1] |
| 3u251 | 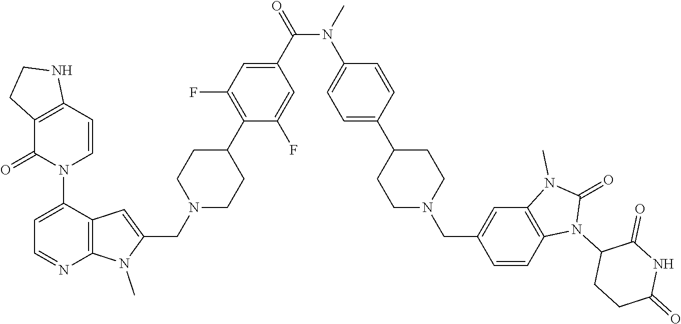 [SMILES: CN(C(=O)c1cc(F)c(C2CCN(Cc3cc4c(-n5ccc6c(c5=O)CCN6)ccnc4n3C)CC2)c(F)c1)c1ccc(C2CCN(Cc3ccc4c(c3)n(C)c(=O)n4C3CCC(=O)NC3=O)CC2)cc1] |
| 3u252 | 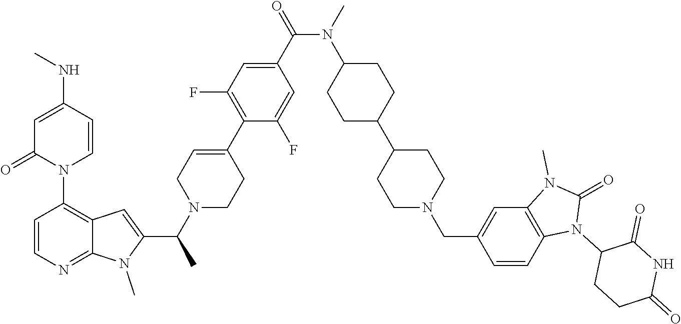 [SMILES: CNc1ccn(-c2ccnc3c2cc([C@H](C)N2CC=C(c4c(F)cc(C(=O)N(C)C5CCC(C6CCN(Cc7ccc8c(c7)n(C)c(=O)n8C7CCC(=O)NC7=O)CC6)CC5)cc4F)CC2)n3C)c(=O)c1] |
| 3u253 | 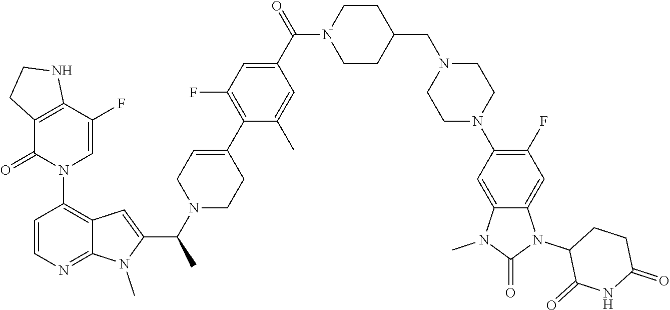 [SMILES: Cc1cc(C(=O)N2CCC(CN3CCN(c4cc5c(cc4F)n(C4CCC(=O)NC4=O)c(=O)n5C)CC3)CC2)cc(F)c1C1=CCN([C@@H](C)c2cc3c(-n4cc(F)c5c(c4=O)CCN5)ccnc3n2C)CC1] |
| 3u254 | 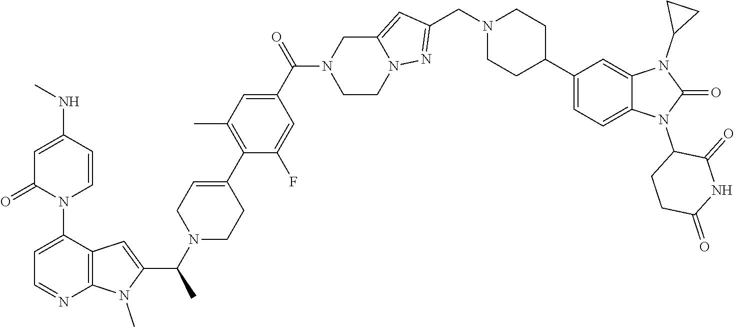 [SMILES: CNc1ccn(-c2ccnc3c2cc([C@H](C)N2CC=C(c4c(C)cc(C(=O)N5CCn6nc(CN7CCC(c8ccc9c(c8)n(C8CC8)c(=O)n9C8CCC(=O)NC8=O)CC7)cc6C5)cc4F)CC2)n3C)c(=O)c1] |
| 3u255 | 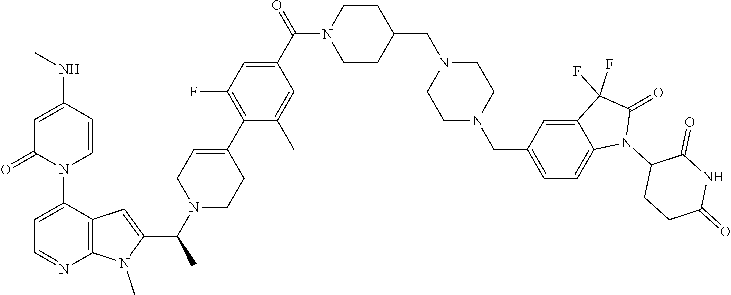 [SMILES: CNc1ccn(-c2ccnc3c2cc([C@H](C)N2CC=C(c4c(C)cc(C(=O)N5CCC(CN6CCN(Cc7ccc8c(c7)C(F)(F)C(=O)N8C7CCC(=O)NC7=O)CC6)CC5)cc4F)CC2)n3C)c(=O)c1] |
| 3u256 | 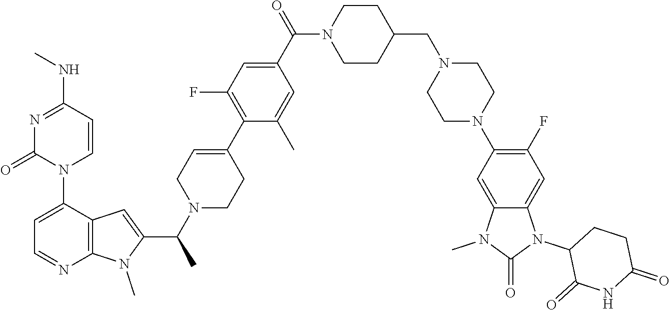 [SMILES: CNc1ccn(-c2ccnc3c2cc([C@H](C)N2CC=C(c4c(C)cc(C(=O)N5CCC(CN6CCN(c7cc8c(cc7F)n(C7CCC(=O)NC7=O)c(=O)n8C)CC6)CC5)cc4F)CC2)n3C)c(=O)n1] |
| 3u257 | 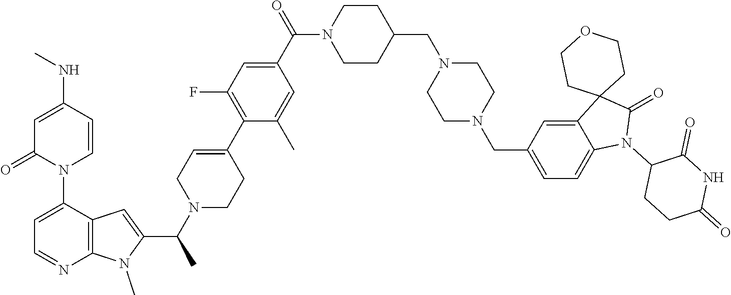 [SMILES: CNc1ccn(-c2ccnc3c2cc([C@H](C)N2CC=C(c4c(C)cc(C(=O)N5CCC(CN6CCN(Cc7ccc8c(c7)C7(CCOCC7)C(=O)N8C7CCC(=O)NC7=O)CC6)CC5)cc4F)CC2)n3C)c(=O)c1] |
| 3u258 | 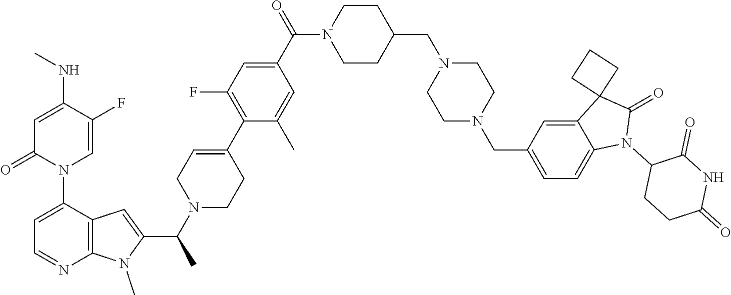 [SMILES: CNc1cc(=O)n(-c2ccnc3c2cc([C@H](C)N2CC=C(c4c(C)cc(C(=O)N5CCC(CN6CCN(Cc7ccc8c(c7)C7(CCC7)C(=O)N8C7CCC(=O)NC7=O)CC6)CC5)cc4F)CC2)n3C)cc1F] |
| 3u259 | 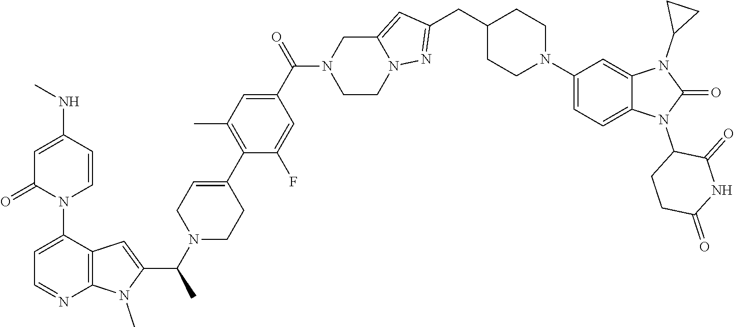 [SMILES: CNc1ccn(-c2ccnc3c2cc([C@H](C)N2CC=C(c4c(C)cc(C(=O)N5CCn6nc(CC7CCN(c8ccc9c(c8)n(C8CC8)c(=O)n9C8CCC(=O)NC8=O)CC7)cc6C5)cc4F)CC2)n3C)c(=O)c1] |
| 3u26 | 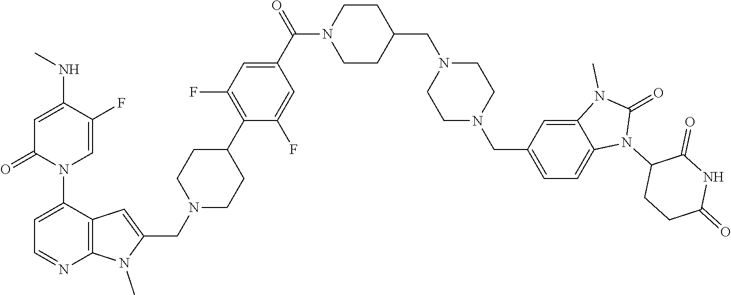 [SMILES: CNc1cc(=O)n(-c2ccnc3c2cc(CN2CCC(c4c(F)cc(C(=O)N5CCC(CN6CCN(Cc7ccc8c(c7)n(C)c(=O)n8C7CCC(=O)NC7=O)CC6)CC5)cc4F)CC2)n3C)cc1F] |
| 3u260 | 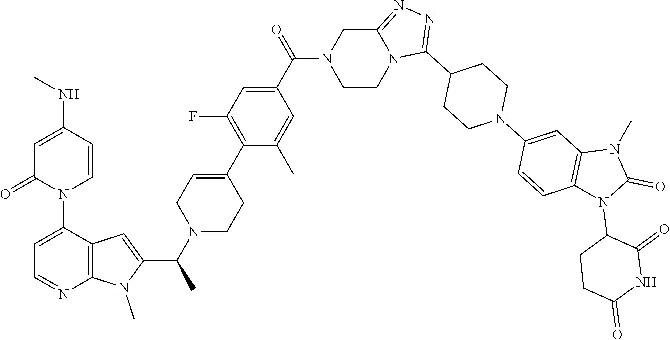 [SMILES: CNc1ccn(-c2ccnc3c2cc([C@H](C)N2CC=C(c4c(C)cc(C(=O)N5CCn6c(nnc6C6CCN(c7ccc8c(c7)n(C)c(=O)n8C7CCC(=O)NC7=O)CC6)C5)cc4F)CC2)n3C)c(=O)c1] |
| 3u261 | 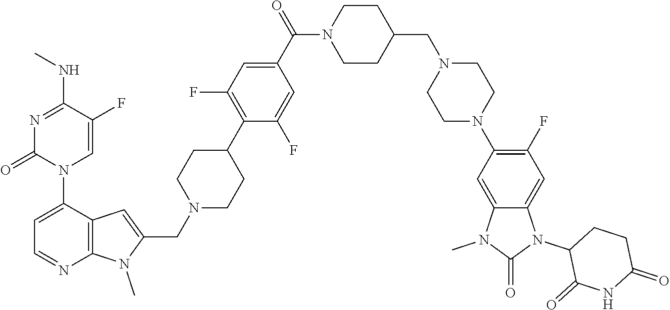 [SMILES: CNc1nc(=O)n(-c2ccnc3c2cc(CN2CCC(c4c(F)cc(C(=O)N5CCC(CN6CCN(c7cc8c(cc7F)n(C7CCC(=O)NC7=O)c(=O)n8C)CC6)CC5)cc4F)CC2)n3C)cc1F] |
| 3u262 | 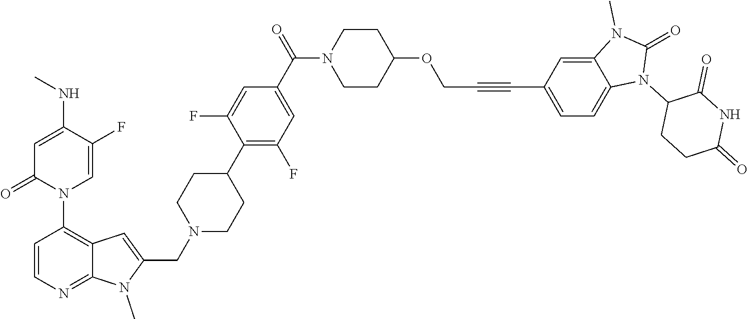 [SMILES: CNc1cc(=O)n(-c2ccnc3c2cc(CN2CCC(c4c(F)cc(C(=O)N5CCC(OCC#Cc6ccc7c(c6)n(C)c(=O)n7C6CCC(=O)NC6=O)CC5)cc4F)CC2)n3C)cc1F] |
| 3u263 | 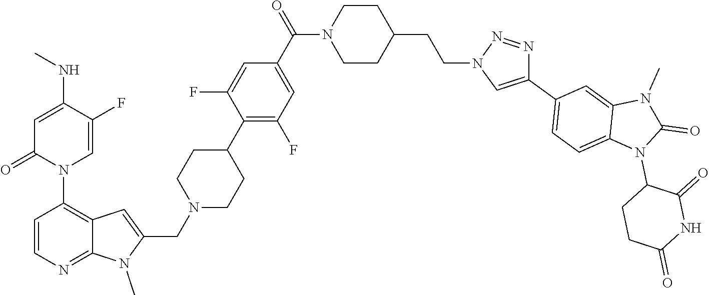 [SMILES: CNc1cc(=O)n(-c2ccnc3c2cc(CN2CCC(c4c(F)cc(C(=O)N5CCC(CCn6cc(-c7ccc8c(c7)n(C)c(=O)n8C7CCC(=O)NC7=O)nn6)CC5)cc4F)CC2)n3C)cc1F] |
| 3u264 | 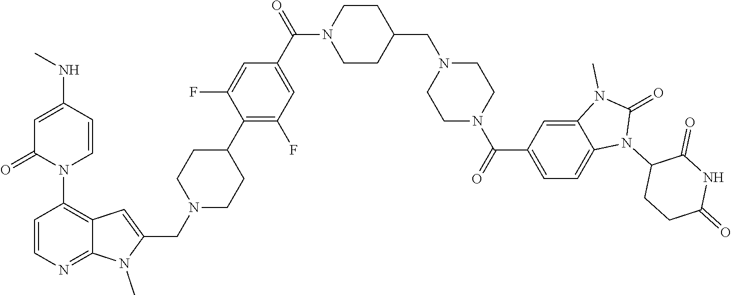 [SMILES: CNc1ccn(-c2ccnc3c2cc(CN2CCC(c4c(F)cc(C(=O)N5CCC(CN6CCN(C(=O)c7ccc8c(c7)n(C)c(=O)n8C7CCC(=O)NC7=O)CC6)CC5)cc4F)CC2)n3C)c(=O)c1] |
| 3u265 | 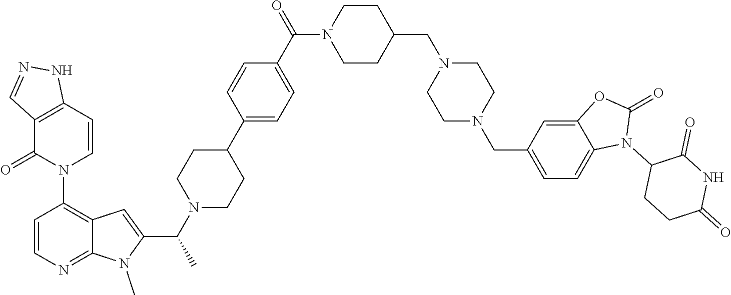 [SMILES: C[C@H](c1cc2c(-n3ccc4[nH]ncc4c3=O)ccnc2n1C)N1CCC(c2ccc(C(=O)N3CCC(CN4CCN(Cc5ccc6c(c5)oc(=O)n6C5CCC(=O)NC5=O)CC4)CC3)cc2)CC1] |
| 3u267 | 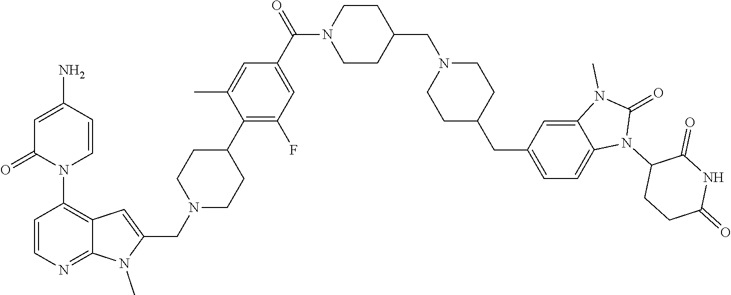 [SMILES: Cc1cc(C(=O)N2CCC(CN3CCC(Cc4ccc5c(c4)n(C)c(=O)n5C4CCC(=O)NC4=O)CC3)CC2)cc(F)c1C1CCN(Cc2cc3c(-n4ccc(N)cc4=O)ccnc3n2C)CC1] |
| 3u268 | 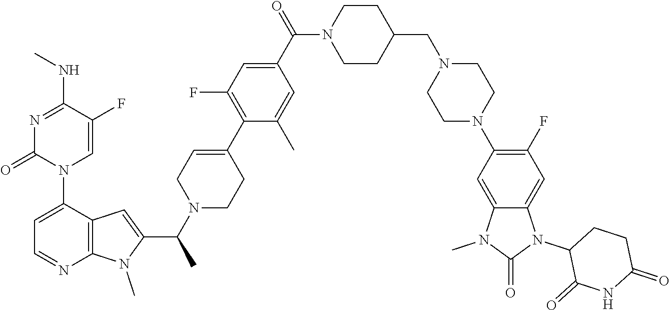 [SMILES: CNc1nc(=O)n(-c2ccnc3c2cc([C@H](C)N2CC=C(c4c(C)cc(C(=O)N5CCC(CN6CCN(c7cc8c(cc7F)n(C7CCC(=O)NC7=O)c(=O)n8C)CC6)CC5)cc4F)CC2)n3C)cc1F] |
| 3u269 | 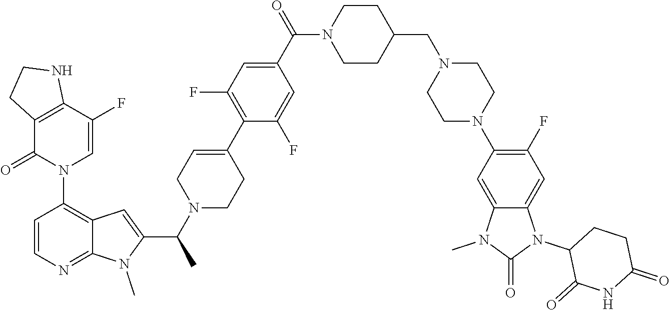 [SMILES: C[C@@H](c1cc2c(-n3cc(F)c4c(c3=O)CCN4)ccnc2n1C)N1CC=C(c2c(F)cc(C(=O)N3CCC(CN4CCN(c5cc6c(cc5F)n(C5CCC(=O)NC5=O)c(=O)n6C)CC4)CC3)cc2F)CC1] |
| 3u27 and 3u28 | 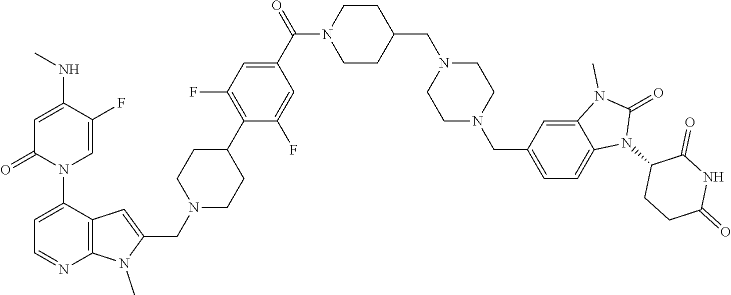 [SMILES: CNc1cc(=O)n(-c2ccnc3c2cc(CN2CCC(c4c(F)cc(C(=O)N5CCC(CN6CCN(Cc7ccc8c(c7)n(C)c(=O)n8[C@H]7CCC(=O)NC7=O)CC6)CC5)cc4F)CC2)n3C)cc1F] |
| | 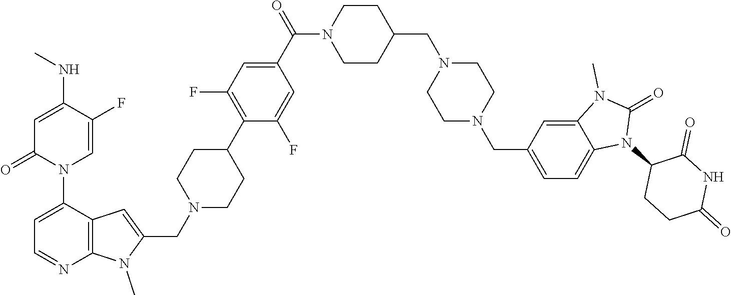 [SMILES: CNc1cc(=O)n(-c2ccnc3c2cc(CN2CCC(c4c(F)cc(C(=O)N5CCC(CN6CCN(Cc7ccc8c(c7)n(C)c(=O)n8[C@@H]7CCC(=O)NC7=O)CC6)CC5)cc4F)CC2)n3C)cc1F] |
| 3u270 | 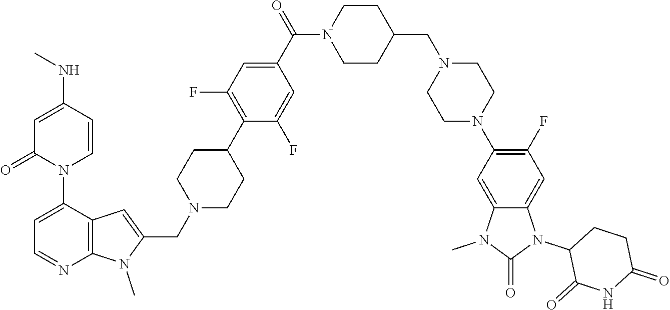 [SMILES: CNc1ccn(-c2ccnc3c2cc(CN2CCC(c4c(F)cc(C(=O)N5CCC(CN6CCN(c7cc8c(cc7F)n(C7CCC(=O)NC7=O)c(=O)n8C)CC6)CC5)cc4F)CC2)n3C)c(=O)c1] |
| 3u271 | 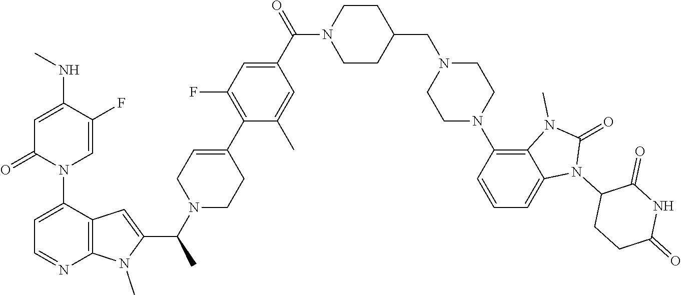 [SMILES: CNc1cc(=O)n(-c2ccnc3c2cc([C@H](C)N2CC=C(c4c(C)cc(C(=O)N5CCC(CN6CCN(c7cccc8c7n(C)c(=O)n8C7CCC(=O)NC7=O)CC6)CC5)cc4F)CC2)n3C)cc1F] |
| 3u272 | 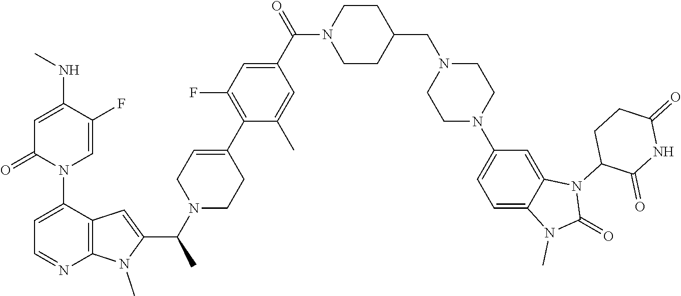 [SMILES: CNc1cc(=O)n(-c2ccnc3c2cc([C@H](C)N2CC=C(c4c(C)cc(C(=O)N5CCC(CN6CCN(c7ccc8c(c7)n(C7CCC(=O)NC7=O)c(=O)n8C)CC6)CC5)cc4F)CC2)n3C)cc1F] |
| 3u273 | 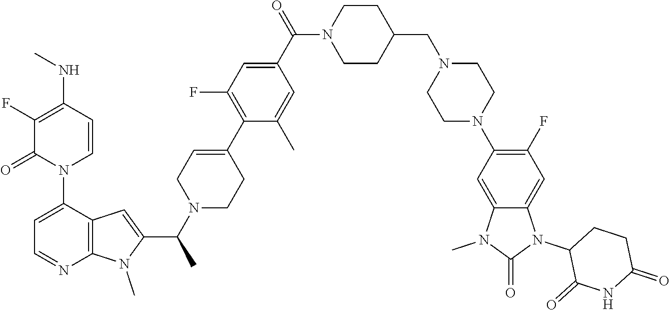 [SMILES: CNc1ccn(-c2ccnc3c2cc([C@H](C)N2CC=C(c4c(C)cc(C(=O)N5CCC(CN6CCN(c7cc8c(cc7F)n(C7CCC(=O)NC7=O)c(=O)n8C)CC6)CC5)cc4F)CC2)n3C)c(=O)c1F] |
| 3u274 | 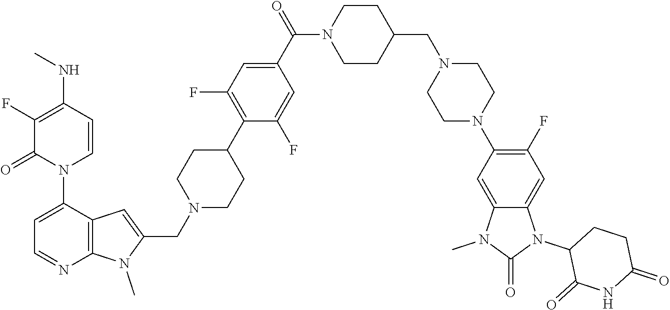 [SMILES: CNc1ccn(-c2ccnc3c2cc(CN2CCC(c4c(F)cc(C(=O)N5CCC(CN6CCN(c7cc8c(cc7F)n(C7CCC(=O)NC7=O)c(=O)n8C)CC6)CC5)cc4F)CC2)n3C)c(=O)c1F] |
| 3u275 | 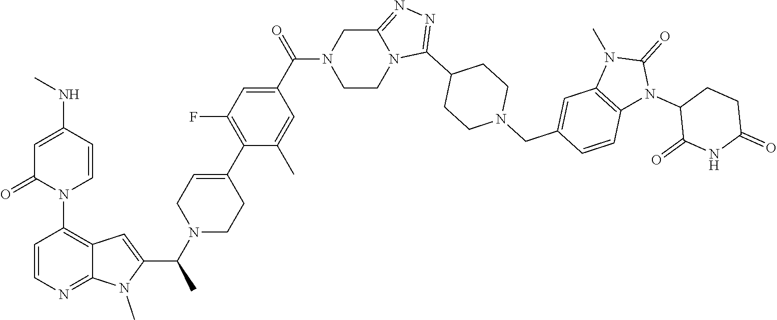 [SMILES: CNc1ccn(-c2ccnc3c2cc([C@H](C)N2CC=C(c4c(C)cc(C(=O)N5CCn6c(nnc6C6CCN(Cc7ccc8c(c7)n(C)c(=O)n8C7CCC(=O)NC7=O)CC6)C5)cc4F)CC2)n3C)c(=O)c1] |
| 3u276 | 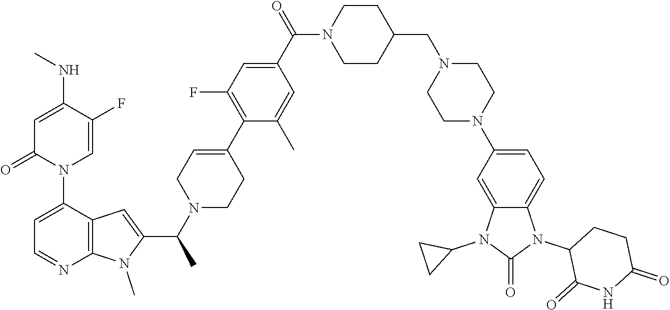 [SMILES: CNc1cc(=O)n(-c2ccnc3c2cc([C@H](C)N2CC=C(c4c(C)cc(C(=O)N5CCC(CN6CCN(c7ccc8c(c7)n(C7CC7)c(=O)n8C7CCC(=O)NC7=O)CC6)CC5)cc4F)CC2)n3C)cc1F] |
| 3u277 | 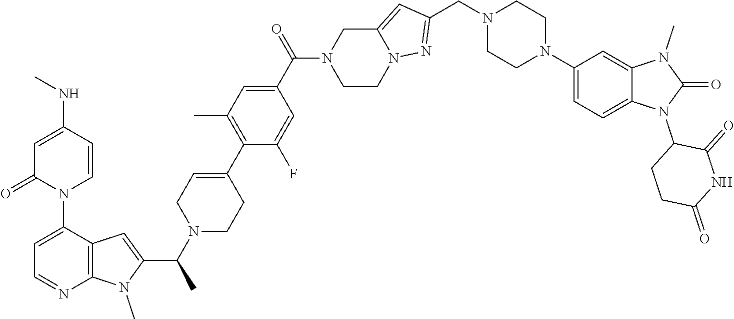 [SMILES: CNc1ccn(-c2ccnc3c2cc([C@H](C)N2CC=C(c4c(C)cc(C(=O)N5CCn6nc(CN7CCN(c8ccc9c(c8)n(C)c(=O)n9C8CCC(=O)NC8=O)CC7)cc6C5)cc4F)CC2)n3C)c(=O)c1] |
| 3u278 and 3u279 | 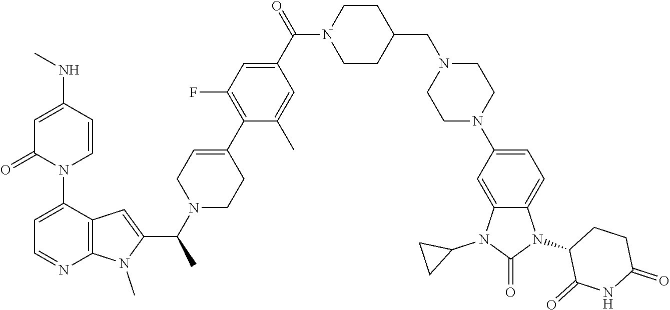 [SMILES: CNc1ccn(-c2ccnc3c2cc([C@H](C)N2CC=C(c4c(C)cc(C(=O)N5CCC(CN6CCN(c7ccc8c(c7)n(C7CC7)c(=O)n8[C@@H]7CCC(=O)NC7=O)CC6)CC5)cc4F)CC2)n3C)c(=O)c1] |
| | 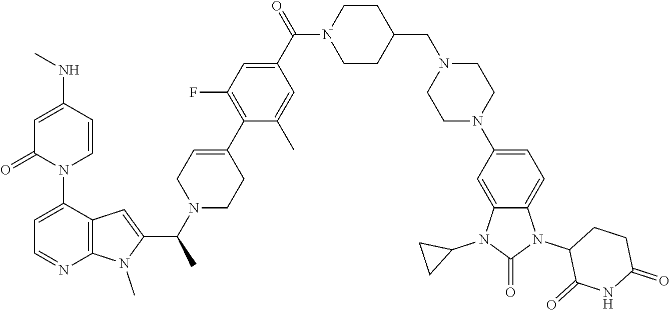 [SMILES: CNc1ccn(-c2ccnc3c2cc([C@H](C)N2CC=C(c4c(C)cc(C(=O)N5CCC(CN6CCN(c7ccc8c(c7)n(C7CC7)c(=O)n8C7CCC(=O)NC7=O)CC6)CC5)cc4F)CC2)n3C)c(=O)c1] |
| 3u280 and 3u281 | 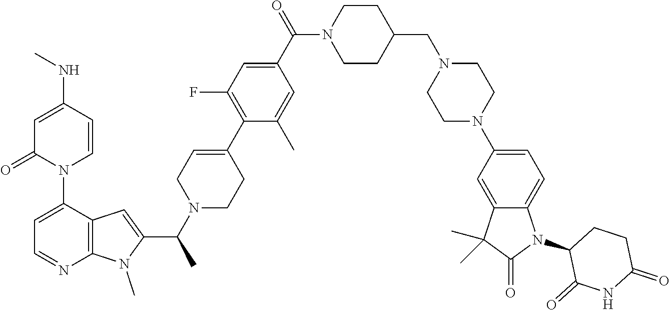 [SMILES: CNc1ccn(-c2ccnc3c2cc([C@H](C)N2CC=C(c4c(C)cc(C(=O)N5CCC(CN6CCN(c7ccc8c(c7)C(C)(C)C(=O)N8[C@H]7CCC(=O)NC7=O)CC6)CC5)cc4F)CC2)n3C)c(=O)c1] |
| | 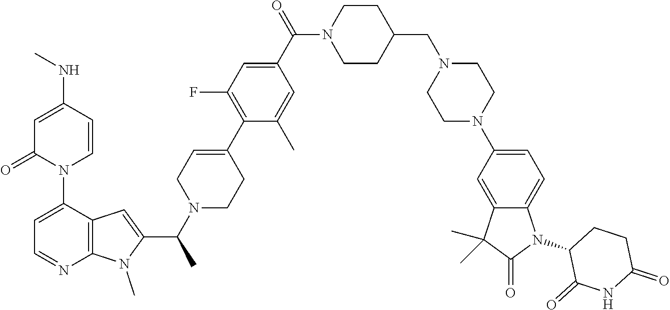 [SMILES: CNc1ccn(-c2ccnc3c2cc([C@H](C)N2CC=C(c4c(C)cc(C(=O)N5CCC(CN6CCN(c7ccc8c(c7)C(C)(C)C(=O)N8[C@@H]7CCC(=O)NC7=O)CC6)CC5)cc4F)CC2)n3C)c(=O)c1] |
| 3u282 | 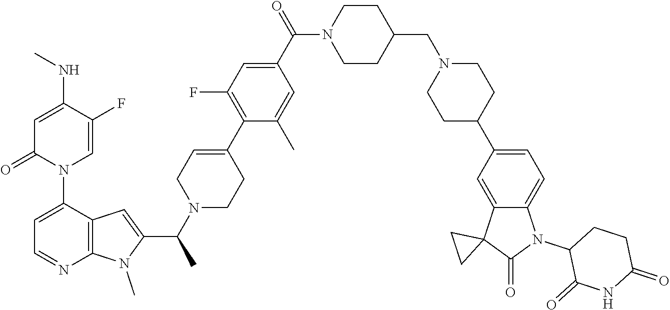 [SMILES: CNc1cc(=O)n(-c2ccnc3c2cc([C@H](C)N2CC=C(c4c(C)cc(C(=O)N5CCC(CN6CCC(c7ccc8c(c7)C7(CC7)C(=O)N8C7CCC(=O)NC7=O)CC6)CC5)cc4F)CC2)n3C)cc1F] |
| 3u283 | 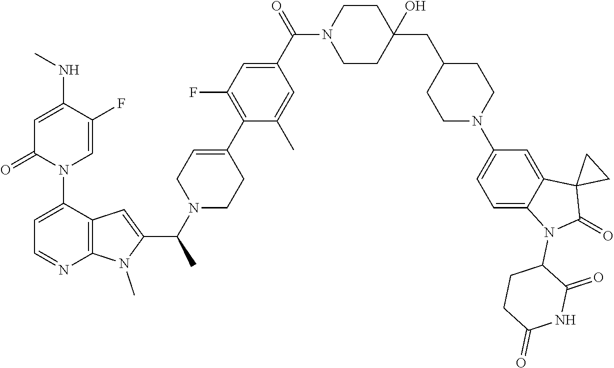 [SMILES: CNc1cc(=O)n(-c2ccnc3c2cc([C@H](C)N2CC=C(c4c(C)cc(C(=O)N5CCC(O)(CC6CCN(c7ccc8c(c7)C7(CC7)C(=O)N8C7CCC(=O)NC7=O)CC6)CC5)cc4F)CC2)n3C)cc1F] |
| 3u284 | 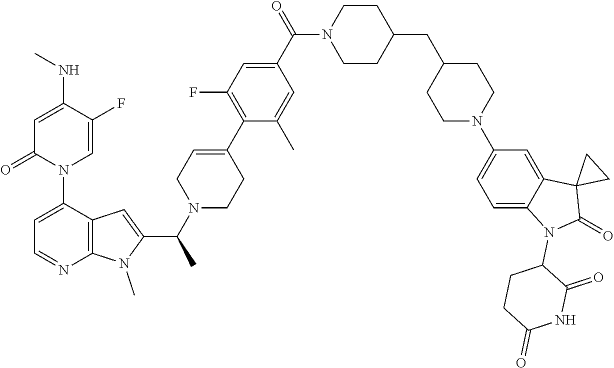 [SMILES: CNc1cc(=O)n(-c2ccnc3c2cc([C@H](C)N2CC=C(c4c(C)cc(C(=O)N5CCC(CC6CCN(c7ccc8c(c7)C7(CC7)C(=O)N8C7CCC(=O)NC7=O)CC6)CC5)cc4F)CC2)n3C)cc1F] |
| 3u285 and 3u286 | 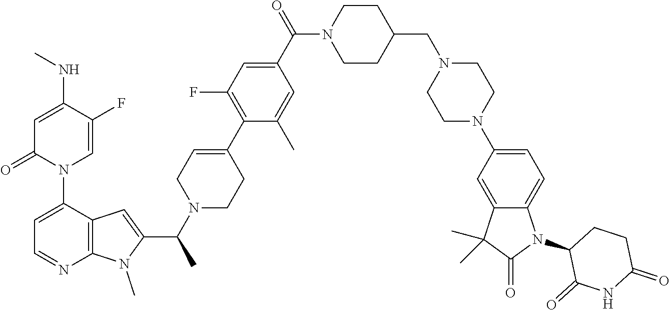 [SMILES: CNc1cc(=O)n(-c2ccnc3c2cc([C@H](C)N2CC=C(c4c(C)cc(C(=O)N5CCC(CN6CCN(c7ccc8c(c7)C(C)(C)C(=O)N8[C@H]7CCC(=O)NC7=O)CC6)CC5)cc4F)CC2)n3C)cc1F] |
| | 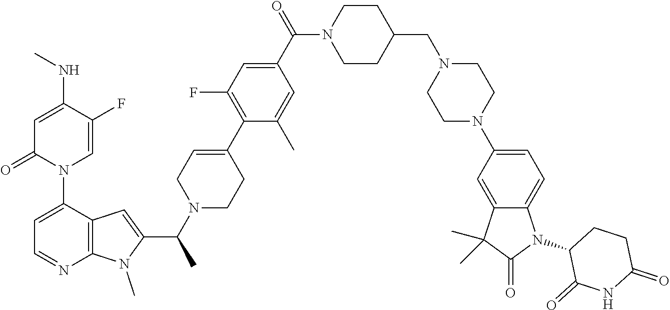 [SMILES: CNc1cc(=O)n(-c2ccnc3c2cc([C@H](C)N2CC=C(c4c(C)cc(C(=O)N5CCC(CN6CCN(c7ccc8c(c7)C(C)(C)C(=O)N8[C@@H]7CCC(=O)NC7=O)CC6)CC5)cc4F)CC2)n3C)cc1F] |
| 3u287 | 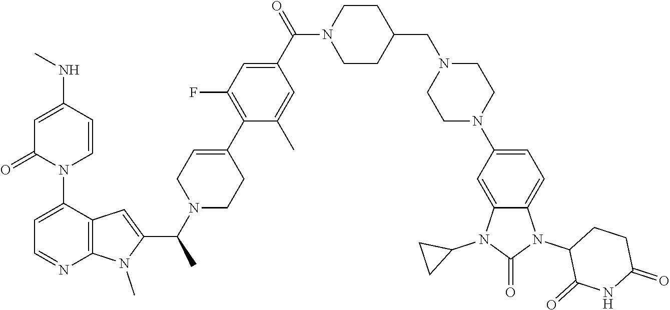 [SMILES: CNc1ccn(-c2ccnc3c2cc([C@H](C)N2CC=C(c4c(C)cc(C(=O)N5CCC(CN6CCN(c7ccc8c(c7)n(C7CC7)c(=O)n8C7CCC(=O)NC7=O)CC6)CC5)cc4F)CC2)n3C)c(=O)c1] |
| 3u288 and 3u289 | 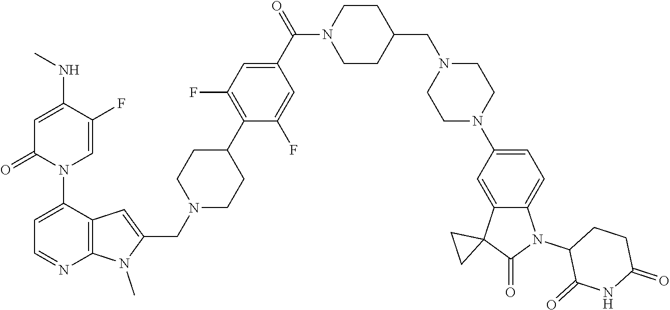 [SMILES: CNc1cc(=O)n(-c2ccnc3c2cc(CN2CCC(c4c(F)cc(C(=O)N5CCC(CN6CCN(c7ccc8c(c7)C7(CC7)C(=O)N8C7CCC(=O)NC7=O)CC6)CC5)cc4F)CC2)n3C)cc1F] |
| | 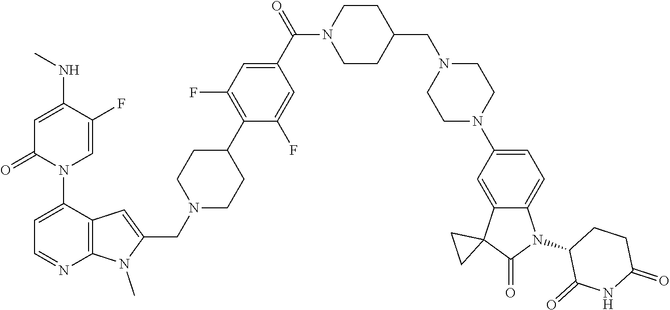 [SMILES: CNc1cc(=O)n(-c2ccnc3c2cc(CN2CCC(c4c(F)cc(C(=O)N5CCC(CN6CCN(c7ccc8c(c7)C7(CC7)C(=O)N8[C@@H]7CCC(=O)NC7=O)CC6)CC5)cc4F)CC2)n3C)cc1F] |
| 3u29 | 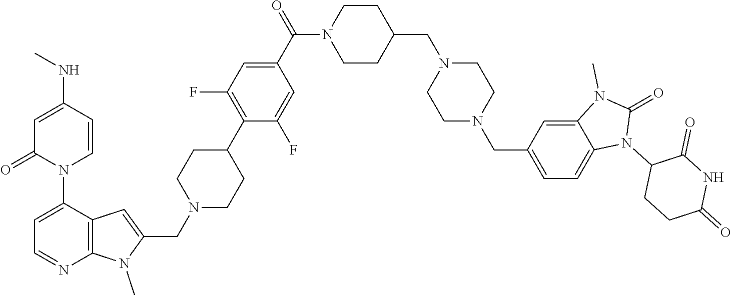 [SMILES: CNc1ccn(-c2ccnc3c2cc(CN2CCC(c4c(F)cc(C(=O)N5CCC(CN6CCN(Cc7ccc8c(c7)n(C)c(=O)n8C7CCC(=O)NC7=O)CC6)CC5)cc4F)CC2)n3C)c(=O)c1] |
| 3u290 | 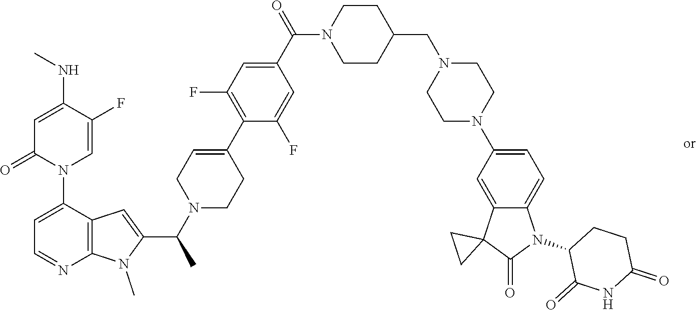 [SMILES: CNc1cc(=O)n(-c2ccnc3c2cc([C@H](C)N2CC=C(c4c(F)cc(C(=O)N5CCC(CN6CCN(c7ccc8c(c7)C7(CC7)C(=O)N8[C@@H]7CCC(=O)NC7=O)CC6)CC5)cc4F)CC2)n3C)cc1F] |
| | 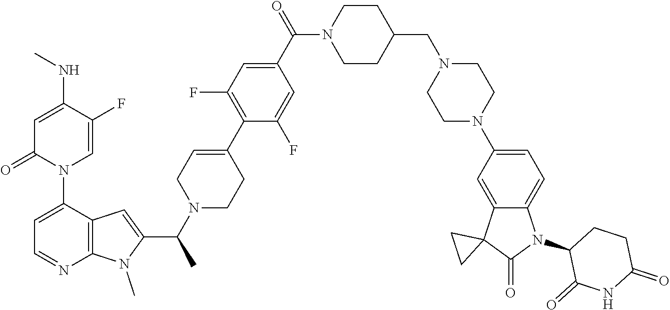 [SMILES: CNc1cc(=O)n(-c2ccnc3c2cc([C@H](C)N2CC=C(c4c(F)cc(C(=O)N5CCC(CN6CCN(c7ccc8c(c7)C7(CC7)C(=O)N8[C@H]7CCC(=O)NC7=O)CC6)CC5)cc4F)CC2)n3C)cc1F] |
| 3u291 and 3u292 | 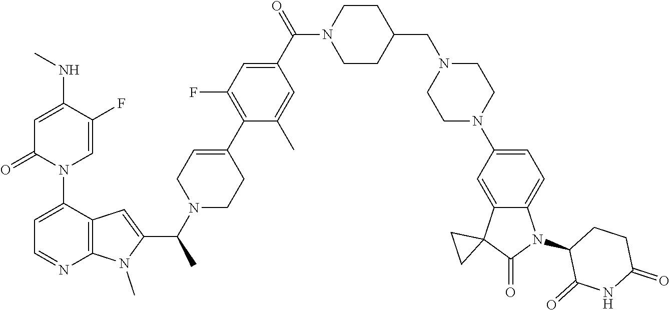 [SMILES: CNc1cc(=O)n(-c2ccnc3c2cc([C@H](C)N2CC=C(c4c(C)cc(C(=O)N5CCC(CN6CCN(c7ccc8c(c7)C7(CC7)C(=O)N8[C@H]7CCC(=O)NC7=O)CC6)CC5)cc4F)CC2)n3C)cc1F] |
| | 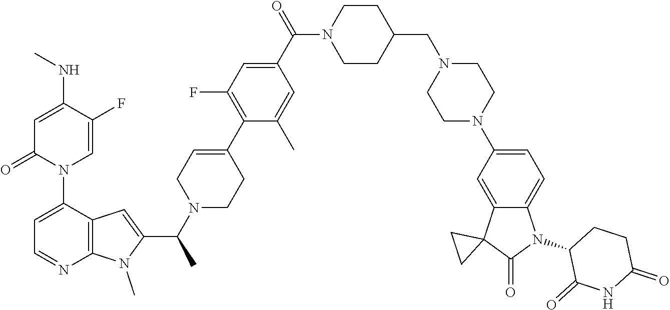 [SMILES: CNc1cc(=O)n(-c2ccnc3c2cc([C@H](C)N2CC=C(c4c(C)cc(C(=O)N5CCC(CN6CCN(c7ccc8c(c7)C7(CC7)C(=O)N8[C@@H]7CCC(=O)NC7=O)CC6)CC5)cc4F)CC2)n3C)cc1F] |
| 2u293 and 3u294 | 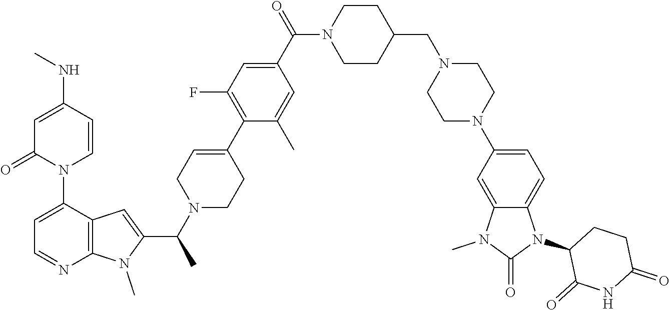 [SMILES: CNc1ccn(-c2ccnc3c2cc([C@H](C)N2CC=C(c4c(C)cc(C(=O)N5CCC(CN6CCN(c7ccc8c(c7)n(C)c(=O)n8[C@H]7CCC(=O)NC7=O)CC6)CC5)cc4F)CC2)n3C)c(=O)c1] |
| | 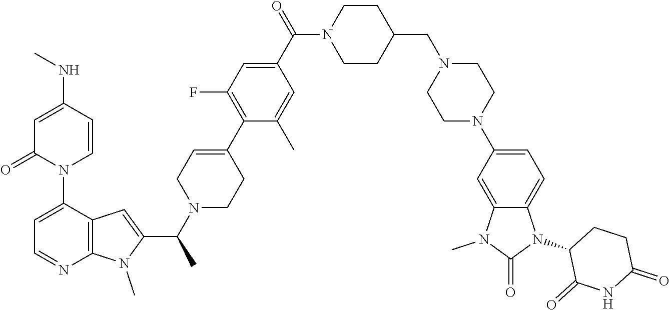 [SMILES: CNc1ccn(-c2ccnc3c2cc([C@H](C)N2CC=C(c4c(C)cc(C(=O)N5CCC(CN6CCN(c7ccc8c(c7)n(C)c(=O)n8[C@@H]7CCC(=O)NC7=O)CC6)CC5)cc4F)CC2)n3C)c(=O)c1] |
| 3u295 | 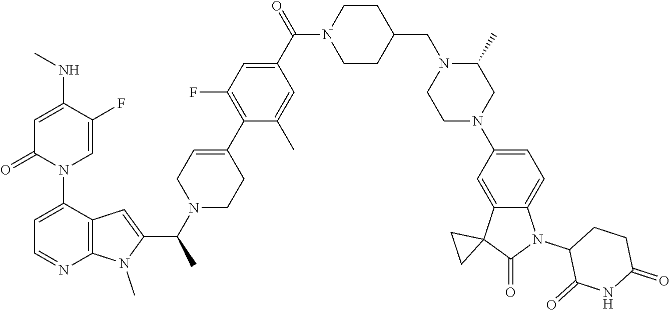 [SMILES: CNc1cc(=O)n(-c2ccnc3c2cc([C@H](C)N2CC=C(c4c(C)cc(C(=O)N5CCC(CN6CCN(c7ccc8c(c7)C7(CC7)C(=O)N8C7CCC(=O)NC7=O)C[C@H]6C)CC5)cc4F)CC2)n3C)cc1F] |
| 3u296 and 3u297 | 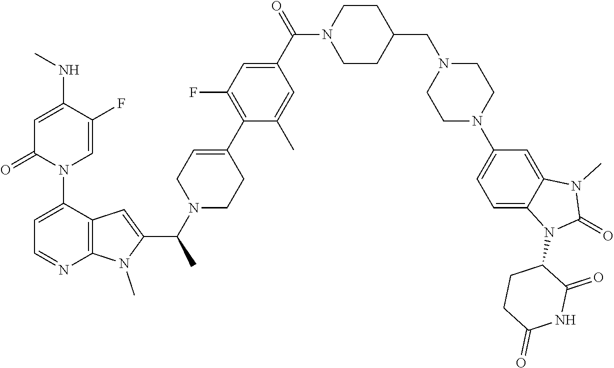 [SMILES: CNc1cc(=O)n(-c2ccnc3c2cc([C@H](C)N2CC=C(c4c(C)cc(C(=O)N5CCC(CN6CCN(c7ccc8c(c7)n(C)c(=O)n8[C@H]7CCC(=O)NC7=O)CC6)CC5)cc4F)CC2)n3C)cc1F] |
| | 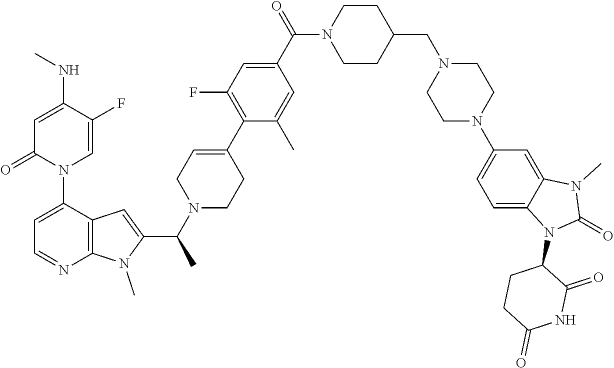 [SMILES: CNc1cc(=O)n(-c2ccnc3c2cc([C@H](C)N2CC=C(c4c(C)cc(C(=O)N5CCC(CN6CCN(c7ccc8c(c7)n(C)c(=O)n8[C@@H]7CCC(=O)NC7=O)CC6)CC5)cc4F)CC2)n3C)cc1F] |
| 3u298 | 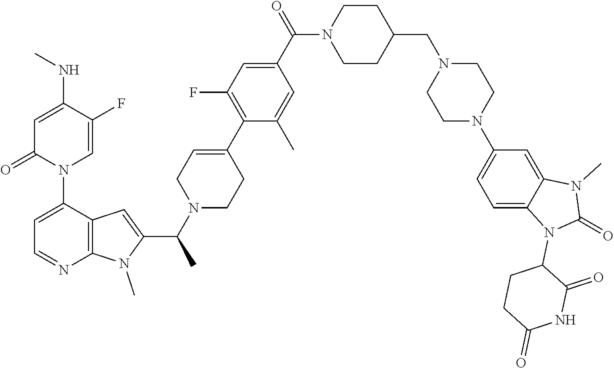 [SMILES: CNc1cc(=O)n(-c2ccnc3c2cc([C@H](C)N2CC=C(c4c(C)cc(C(=O)N5CCC(CN6CCN(c7ccc8c(c7)n(C)c(=O)n8C7CCC(=O)NC7=O)CC6)CC5)cc4F)CC2)n3C)cc1F] |
| 3u299 | 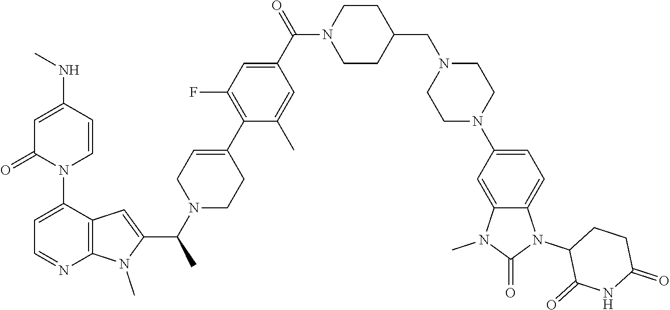 [SMILES: CNc1ccn(-c2ccnc3c2cc([C@H](C)N2CC=C(c4c(C)cc(C(=O)N5CCC(CN6CCN(c7ccc8c(c7)n(C)c(=O)n8C7CCC(=O)NC7=O)CC6)CC5)cc4F)CC2)n3C)c(=O)c1] |
| 3u30 and 3u31 | 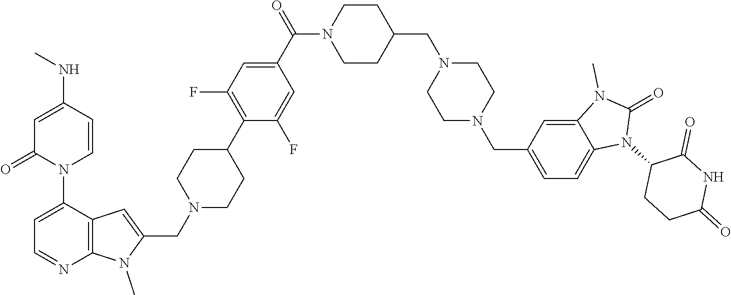 [SMILES: CNc1ccn(-c2ccnc3c2cc(CN2CCC(c4c(F)cc(C(=O)N5CCC(CN6CCN(Cc7ccc8c(c7)n(C)c(=O)n8[C@H]7CCC(=O)NC7=O)CC6)CC5)cc4F)CC2)n3C)c(=O)c1] |
| | 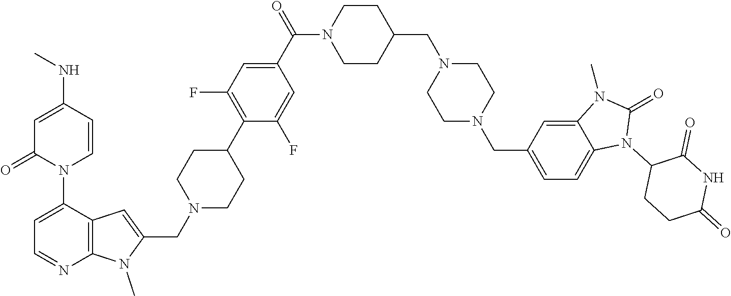 [SMILES: CNc1ccn(-c2ccnc3c2cc(CN2CCC(c4c(F)cc(C(=O)N5CCC(CN6CCN(Cc7ccc8c(c7)n(C)c(=O)n8C7CCC(=O)NC7=O)CC6)CC5)cc4F)CC2)n3C)c(=O)c1] |
| 3u300 | 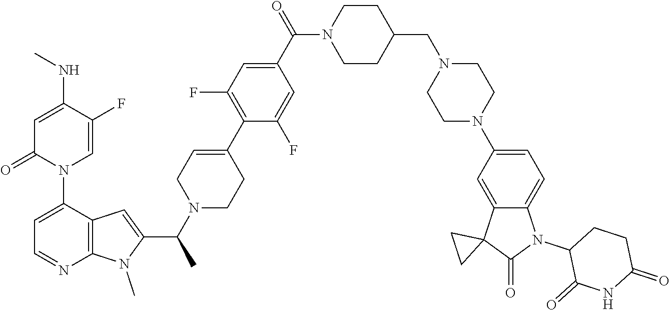 [SMILES: CNc1cc(=O)n(-c2ccnc3c2cc([C@H](C)N2CC=C(c4c(F)cc(C(=O)N5CCC(CN6CCN(c7ccc8c(c7)C7(CC7)C(=O)N8C7CCC(=O)NC7=O)CC6)CC5)cc4F)CC2)n3C)cc1F] |
| 3u301 | 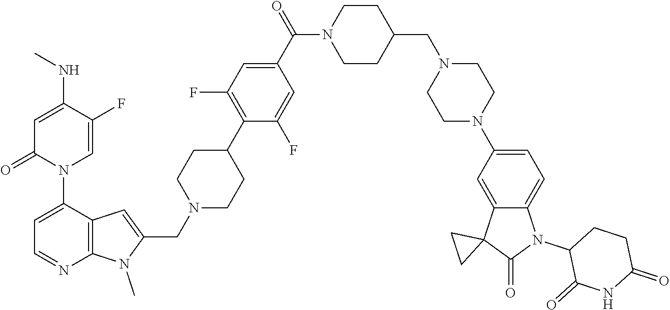 [SMILES: CNc1cc(=O)n(-c2ccnc3c2cc(CN2CCC(c4c(F)cc(C(=O)N5CCC(CN6CCN(c7ccc8c(c7)C7(CC7)C(=O)N8C7CCC(=O)NC7=O)CC6)CC5)cc4F)CC2)n3C)cc1F] |
| 3u302 | 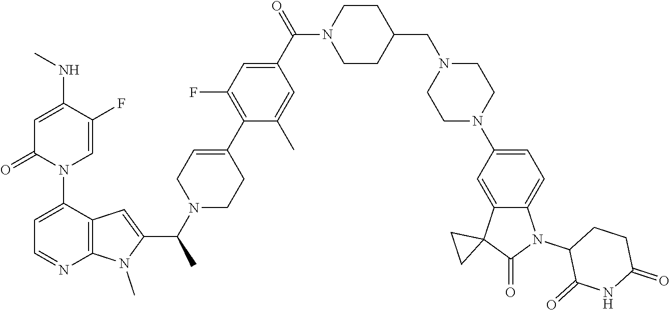 [SMILES: CNc1cc(=O)n(-c2ccnc3c2cc([C@H](C)N2CC=C(c4c(C)cc(C(=O)N5CCC(CN6CCN(c7ccc8c(c7)C7(CC7)C(=O)N8C7CCC(=O)NC7=O)CC6)CC5)cc4F)CC2)n3C)cc1F] |
| 3u303 | 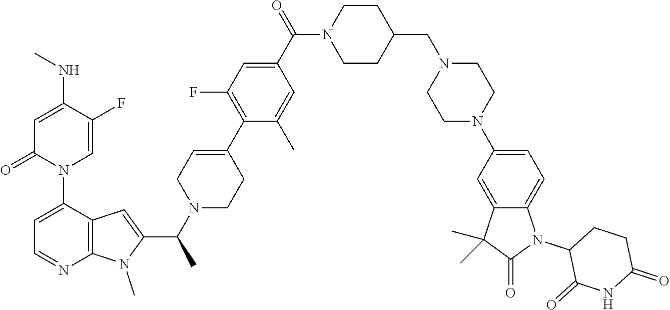 [SMILES: CNc1cc(=O)n(-c2ccnc3c2cc([C@H](C)N2CC=C(c4c(C)cc(C(=O)N5CCC(CN6CCN(c7ccc8c(c7)C(C)(C)C(=O)N8C7CCC(=O)NC7=O)CC6)CC5)cc4F)CC2)n3C)cc1F] |
| 3u304 | 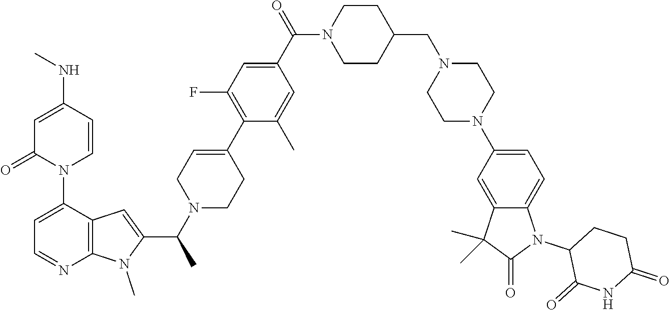 [SMILES: CNc1ccn(-c2ccnc3c2cc([C@H](C)N2CC=C(c4c(C)cc(C(=O)N5CCC(CN6CCN(c7ccc8c(c7)C(C)(C)C(=O)N8C7CCC(=O)NC7=O)CC6)CC5)cc4F)CC2)n3C)c(=O)c1] |
| 3u305 | 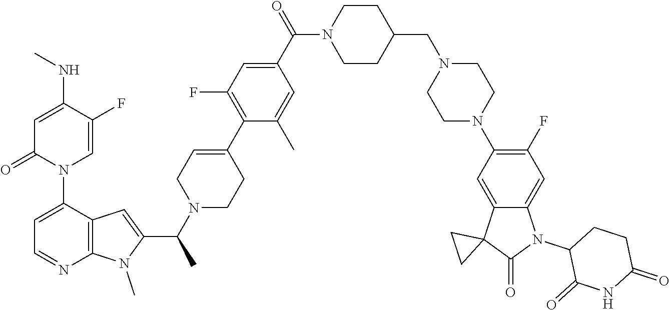 [SMILES: CNc1cc(=O)n(-c2ccnc3c2cc([C@H](C)N2CC=C(c4c(C)cc(C(=O)N5CCC(CN6CCN(c7cc8c(cc7F)N(C7CCC(=O)NC7=O)C(=O)C87CC7)CC6)CC5)cc4F)CC2)n3C)cc1F] |
| 3u306 | 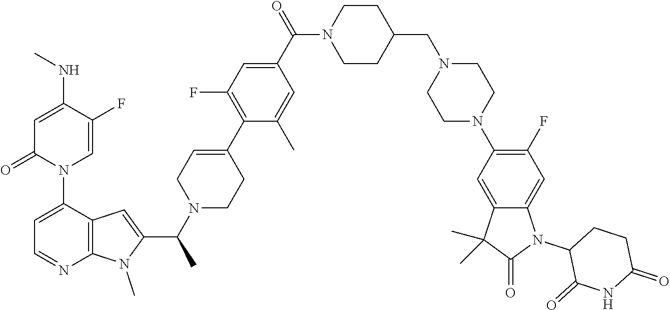 [SMILES: CNc1cc(=O)n(-c2ccnc3c2cc([C@H](C)N2CC=C(c4c(C)cc(C(=O)N5CCC(CN6CCN(c7cc8c(cc7F)N(C7CCC(=O)NC7=O)C(=O)C8(C)C)CC6)CC5)cc4F)CC2)n3C)cc1F] |
| 3u307 and 3u308 | 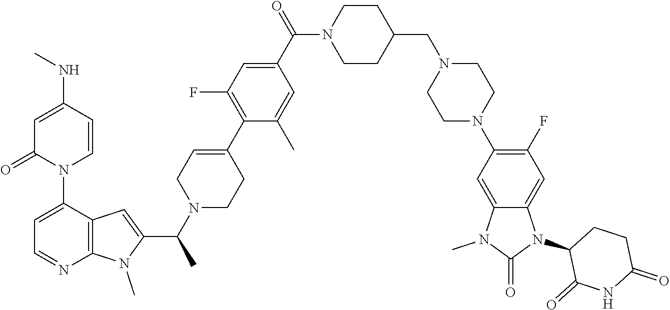 [SMILES: CNc1ccn(-c2ccnc3c2cc([C@H](C)N2CC=C(c4c(C)cc(C(=O)N5CCC(CN6CCN(c7cc8c(cc7F)n([C@H]7CCC(=O)NC7=O)c(=O)n8C)CC6)CC5)cc4F)CC2)n3C)c(=O)c1] |
| | 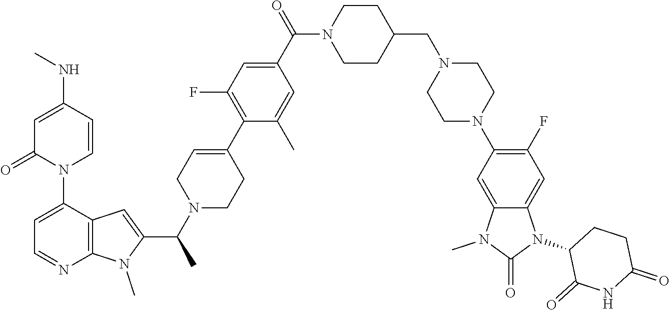 [SMILES: CNc1ccn(-c2ccnc3c2cc([C@H](C)N2CC=C(c4c(C)cc(C(=O)N5CCC(CN6CCN(c7cc8c(cc7F)n([C@@H]7CCC(=O)NC7=O)c(=O)n8C)CC6)CC5)cc4F)CC2)n3C)c(=O)c1] |
| 3u309 | 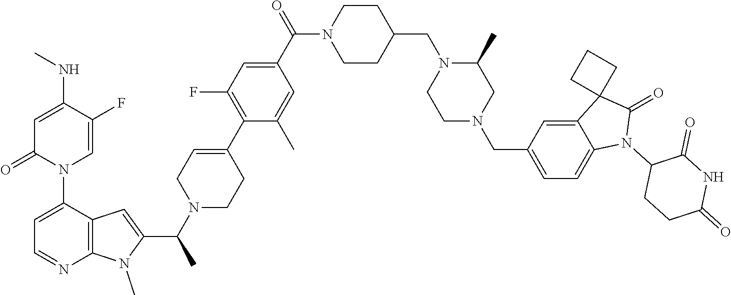 [SMILES: CNc1cc(=O)n(-c2ccnc3c2cc([C@H](C)N2CC=C(c4c(C)cc(C(=O)N5CCC(CN6CCN(Cc7ccc8c(c7)C7(CCC7)C(=O)N8C7CCC(=O)NC7=O)C[C@@H]6C)CC5)cc4F)CC2)n3C)cc1F] |
| 3u310 | 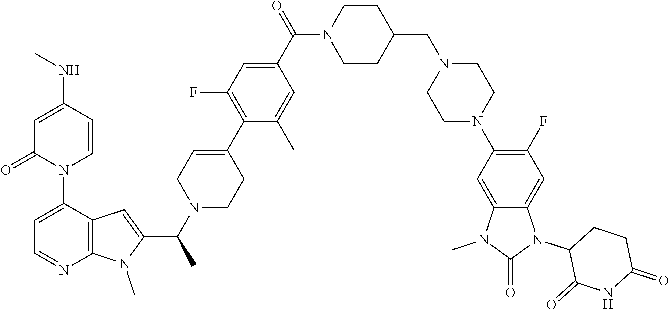 [SMILES: CNc1ccn(-c2ccnc3c2cc([C@H](C)N2CC=C(c4c(C)cc(C(=O)N5CCC(CN6CCN(c7cc8c(cc7F)n(C7CCC(=O)NC7=O)c(=O)n8C)CC6)CC5)cc4F)CC2)n3C)c(=O)c1] |
| 3u311 | 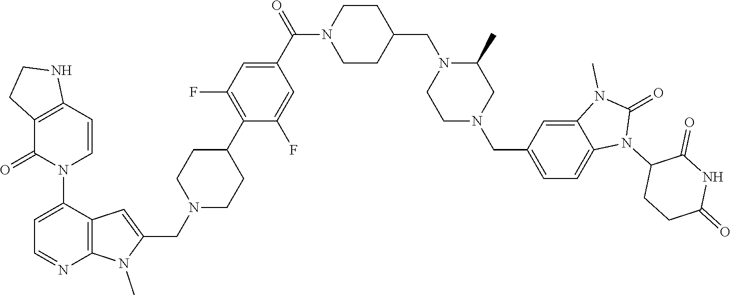 [SMILES: C[C@H]1CN(Cc2ccc3c(c2)n(C)c(=O)n3C2CCC(=O)NC2=O)CCN1CC1CCN(C(=O)c2cc(F)c(C3CCN(Cc4cc5c(-n6ccc7c(c6=O)CCN7)ccnc5n4C)CC3)c(F)c2)CC1] |
| 3u32 | 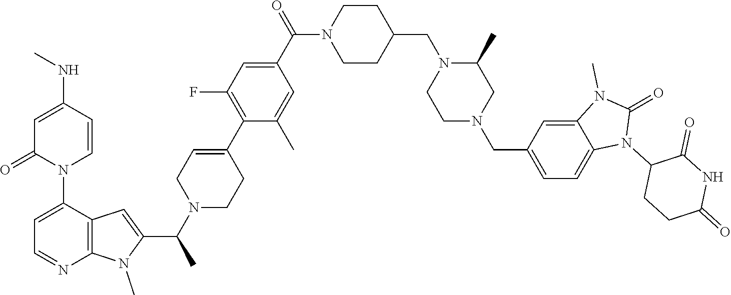 [SMILES: CNc1ccn(-c2ccnc3c2cc([C@H](C)N2CC=C(c4c(C)cc(C(=O)N5CCC(CN6CCN(Cc7ccc8c(c7)n(C)c(=O)n8C7CCC(=O)NC7=O)C[C@@H]6C)CC5)cc4F)CC2)n3C)c(=O)c1] |
| 3u33 and 3u34 | 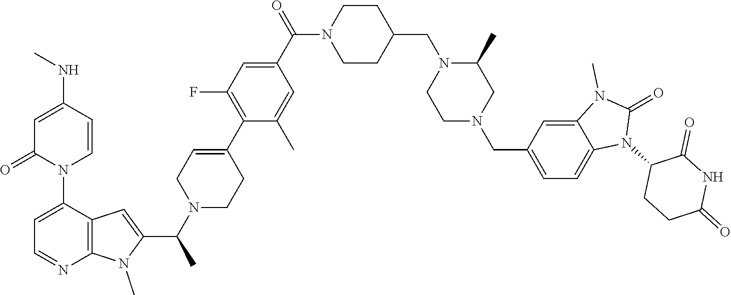 [SMILES: CNc1ccn(-c2ccnc3c2cc([C@H](C)N2CC=C(c4c(C)cc(C(=O)N5CCC(CN6CCN(Cc7ccc8c(c7)n(C)c(=O)n8[C@H]7CCC(=O)NC7=O)C[C@@H]6C)CC5)cc4F)CC2)n3C)c(=O)c1] |
| | 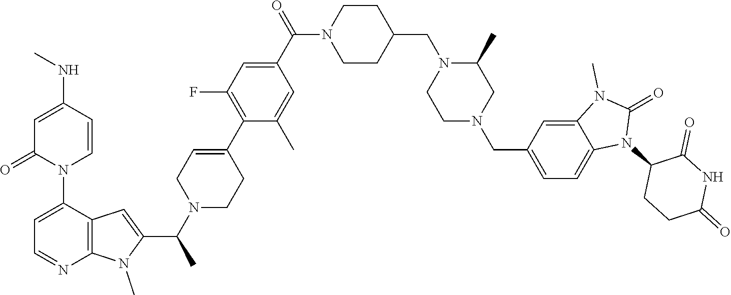 [SMILES: CNc1ccn(-c2ccnc3c2cc([C@H](C)N2CC=C(c4c(C)cc(C(=O)N5CCC(CN6CCN(Cc7ccc8c(c7)n(C)c(=O)n8[C@@H]7CCC(=O)NC7=O)C[C@@H]6C)CC5)cc4F)CC2)n3C)c(=O)c1] |
| 3u35 | 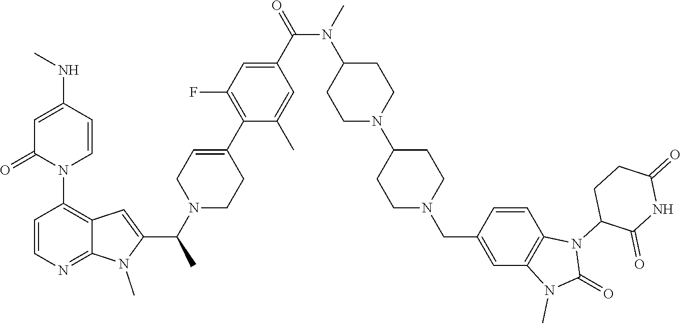 [SMILES: CNc1ccn(-c2ccnc3c2cc([C@H](C)N2CC=C(c4c(C)cc(C(=O)N(C)C5CCN(C6CCN(Cc7ccc8c(c7)n(C)c(=O)n8C7CCC(=O)NC7=O)CC6)CC5)cc4F)CC2)n3C)c(=O)c1] |
| 3u36 | 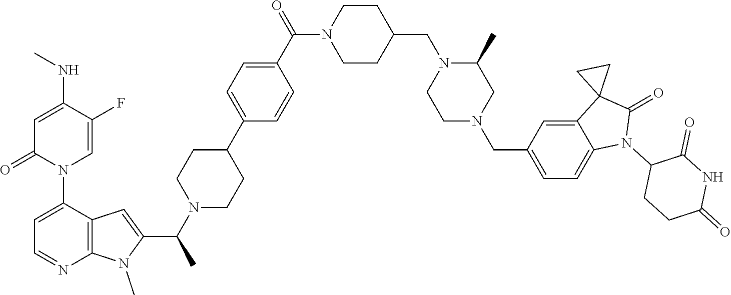 [SMILES: CNc1cc(=O)n(-c2ccnc3c2cc([C@H](C)N2CCC(c4ccc(C(=O)N5CCC(CN6CCN(Cc7ccc8c(c7)C7(CC7)C(=O)N8C7CCC(=O)NC7=O)C[C@@H]6C)CC5)cc4)CC2)n3C)cc1F] |
| 3u37 and 3u38 | 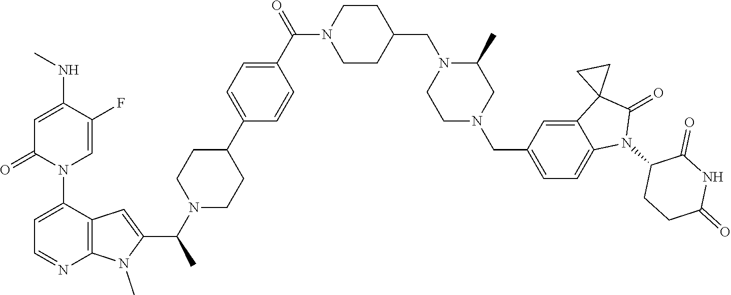 [SMILES: CNc1cc(=O)n(-c2ccnc3c2cc([C@H](C)N2CCC(c4ccc(C(=O)N5CCC(CN6CCN(Cc7ccc8c(c7)C7(CC7)C(=O)N8[C@H]7CCC(=O)NC7=O)C[C@@H]6C)CC5)cc4)CC2)n3C)cc1F] |
| | 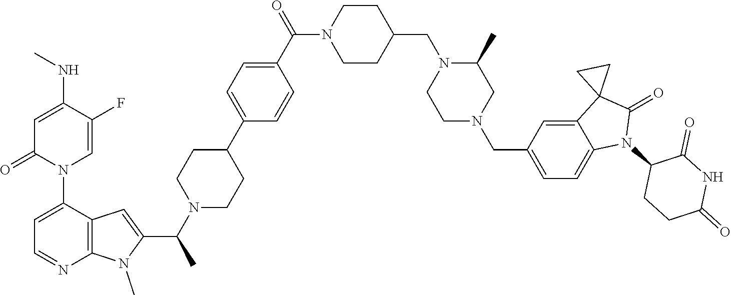 [SMILES: CNc1cc(=O)n(-c2ccnc3c2cc([C@H](C)N2CCC(c4ccc(C(=O)N5CCC(CN6CCN(Cc7ccc8c(c7)C7(CC7)C(=O)N8[C@@H]7CCC(=O)NC7=O)C[C@@H]6C)CC5)cc4)CC2)n3C)cc1F] |
| 3u39 | 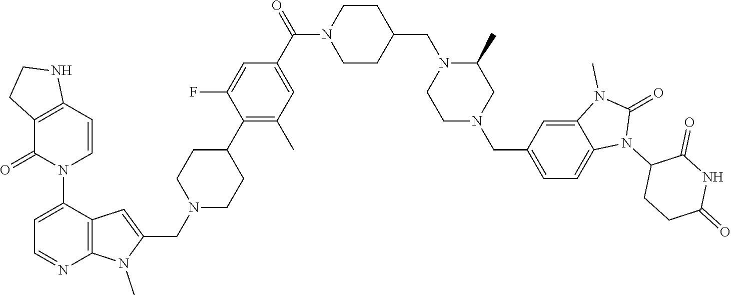 [SMILES: Cc1cc(C(=O)N2CCC(CN3CCN(Cc4ccc5c(c4)n(C)c(=O)n5C4CCC(=O)NC4=O)C[C@@H]3C)CC2)cc(F)c1C1CCN(Cc2cc3c(-n4ccc5c(c4=O)CCN5)ccnc3n2C)CC1] |
| 3u4 | 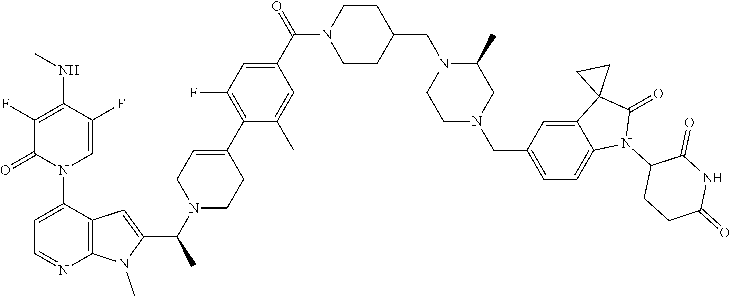 [SMILES: CNc1c(F)cn(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)c(=O)c1F] |
| 3u40 and 3u41 | 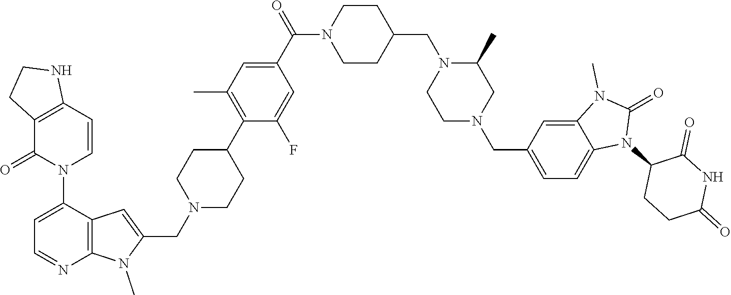 [SMILES: Cc1cc(C(=O)N2CCC(CN3CCN(Cc4ccc5c(c4)n(C)c(=O)n5[C@@H]4CCC(=O)NC4=O)C[C@@H]3C)CC2)cc(F)c1C1CCN(Cc2cc3c(-n4ccc5c(c4=O)CCN5)ccnc3n2C)CC1] |
| | 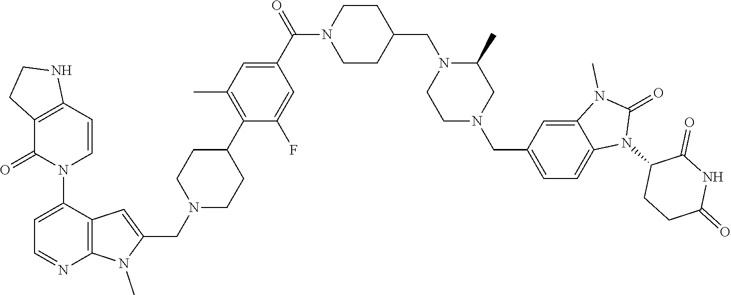 [SMILES: Cc1cc(C(=O)N2CCC(CN3CCN(Cc4ccc5c(c4)n(C)c(=O)n5[C@H]4CCC(=O)NC4=O)C[C@@H]3C)CC2)cc(F)c1C1CCN(Cc2cc3c(-n4ccc5c(c4=O)CCN5)ccnc3n2C)CC1] |
| 3u42 | 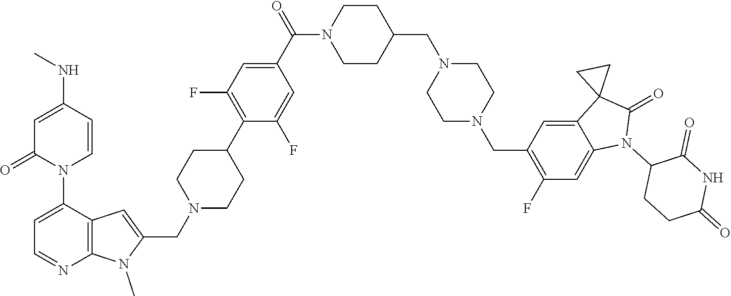 [SMILES: CNc1ccn(-c2ccnc3c2cc(CN2CCC(c4c(F)cc(C(=O)N5CCC(CN6CCN(Cc7cc8c(cc7F)N(C7CCC(=O)NC7=O)C(=O)C87CC7)CC6)CC5)cc4F)CC2)n3C)c(=O)c1] |
| 3u43 and 3u44 | 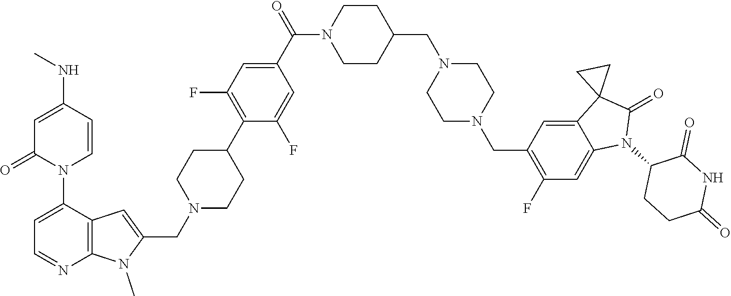 [SMILES: CNc1ccn(-c2ccnc3c2cc(CN2CCC(c4c(F)cc(C(=O)N5CCC(CN6CCN(Cc7cc8c(cc7F)N([C@H]7CCC(=O)NC7=O)C(=O)C87CC7)CC6)CC5)cc4F)CC2)n3C)c(=O)c1] |
| | 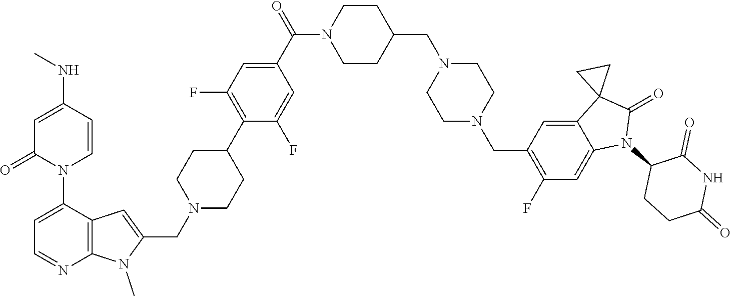 [SMILES: CNc1ccn(-c2ccnc3c2cc(CN2CCC(c4c(F)cc(C(=O)N5CCC(CN6CCN(Cc7cc8c(cc7F)N([C@@H]7CCC(=O)NC7=O)C(=O)C87CC7)CC6)CC5)cc4F)CC2)n3C)c(=O)c1] |
| 3u45 | 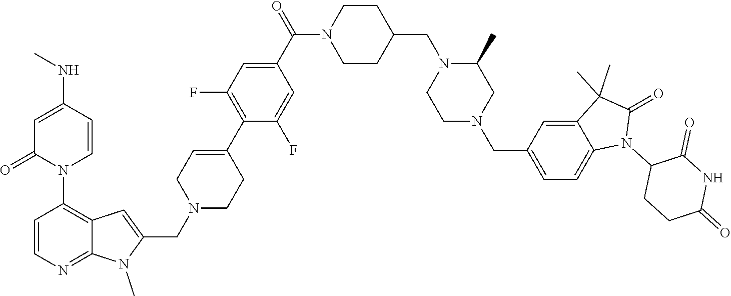 [SMILES: CNc1ccn(-c2ccnc3c2cc(CN2CC=C(c4c(F)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)c(=O)c1] |
| 3u46 and 3u47 | 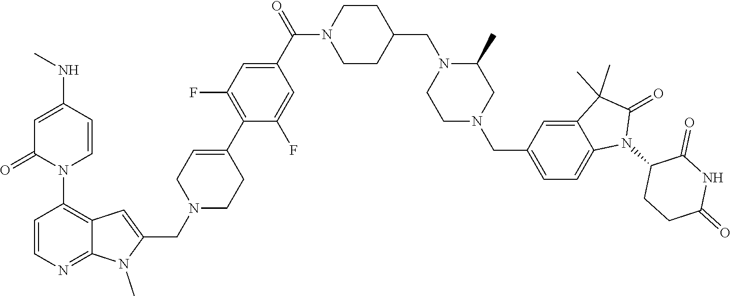 [SMILES: CNc1ccn(-c2ccnc3c2cc(CN2CC=C(c4c(F)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)c(=O)c1] |
| | 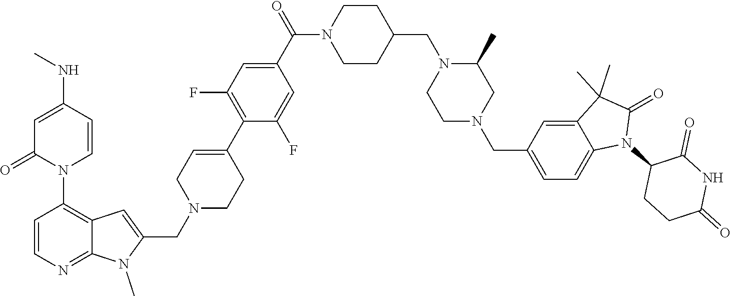 [SMILES: CNc1ccn(-c2ccnc3c2cc(CN2CC=C(c4c(F)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)c(=O)c1] |
| 3u48 | 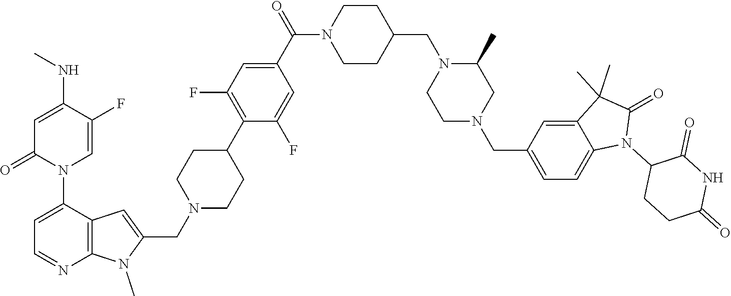 [SMILES: CNc1cc(=O)n(-c2ccnc3c2cc(CN2CCC(c4c(F)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)cc1F] |
| 3u49 and 3u50 | 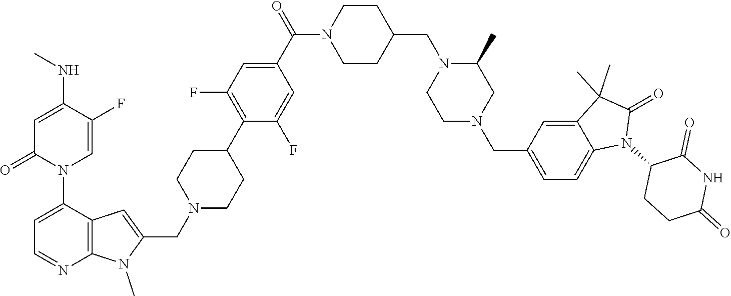 [SMILES: CNc1cc(=O)n(-c2ccnc3c2cc(CN2CCC(c4c(F)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)cc1F] |
| | 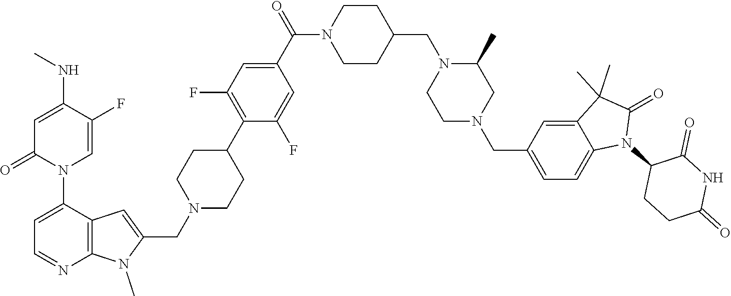 [SMILES: CNc1cc(=O)n(-c2ccnc3c2cc(CN2CCC(c4c(F)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)cc1F] |
| 3u5 | 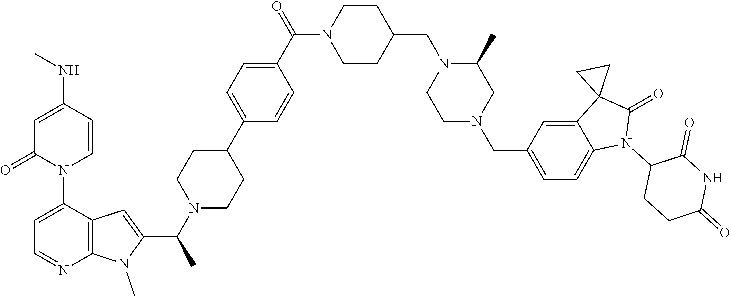 [SMILES: CNc1ccn(-c2ccnc3c2cc([C@H](C)N2CCC(c4ccc(C(=O)N5CCC(CN6CCN(Cc7ccc8c(c7)C7(CC7)C(=O)N8C7CCC(=O)NC7=O)C[C@@H]6C)CC5)cc4)CC2)n3C)c(=O)c1] |
| 3u51 | 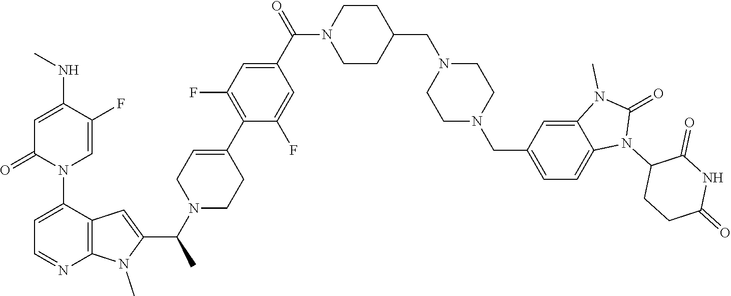 [SMILES: CNc1cc(=O)n(-c2ccnc3c2cc([C@H](C)N2CC=C(c4c(F)cc(C(=O)N5CCC(CN6CCN(Cc7ccc8c(c7)n(C)c(=O)n8C7CCC(=O)NC7=O)CC6)CC5)cc4F)CC2)n3C)cc1F] |
| 3u52 and 3u53 | 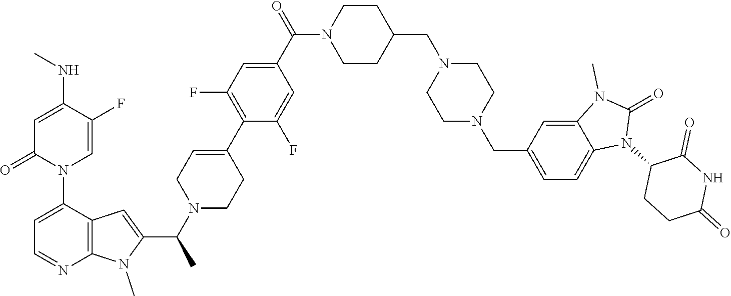 [SMILES: CNc1cc(=O)n(-c2ccnc3c2cc([C@H](C)N2CC=C(c4c(F)cc(C(=O)N5CCC(CN6CCN(Cc7ccc8c(c7)n(C)c(=O)n8[C@H]7CCC(=O)NC7=O)CC6)CC5)cc4F)CC2)n3C)cc1F] |
| | 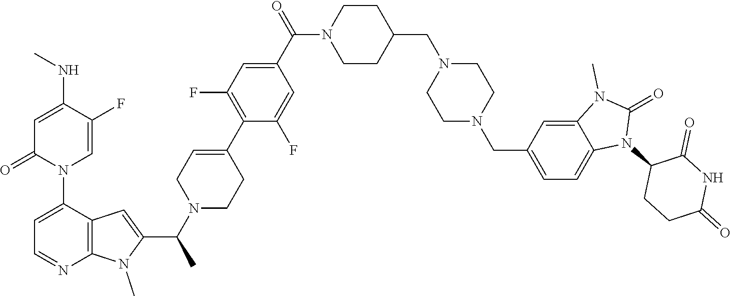 [SMILES: CNc1cc(=O)n(-c2ccnc3c2cc([C@H](C)N2CC=C(c4c(F)cc(C(=O)N5CCC(CN6CCN(Cc7ccc8c(c7)n(C)c(=O)n8[C@@H]7CCC(=O)NC7=O)CC6)CC5)cc4F)CC2)n3C)cc1F] |
| 3u54 | 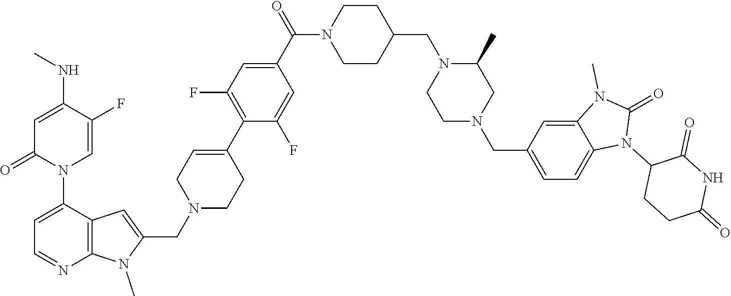 [SMILES: CNc1cc(=O)n(-c2ccnc3c2cc(CN2CC=C(c4c(F)cc(C(=O)N5CCC(CN6CCN(Cc7ccc8c(c7)n(C)c(=O)n8C7CCC(=O)NC7=O)C[C@@H]6C)CC5)cc4F)CC2)n3C)cc1F] |
| 3u55 and 3u56 | 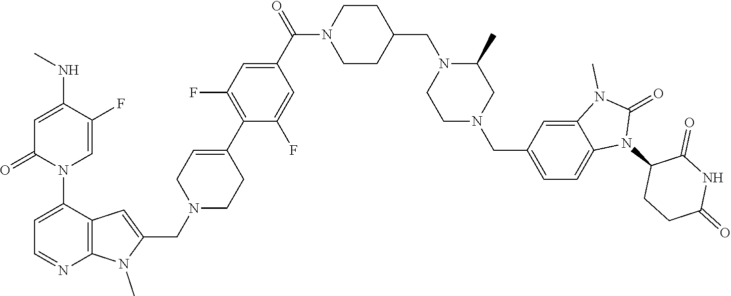 [SMILES: CNc1cc(=O)n(-c2ccnc3c2cc(CN2CC=C(c4c(F)cc(C(=O)N5CCC(CN6CCN(Cc7ccc8c(c7)n(C)c(=O)n8[C@@H]7CCC(=O)NC7=O)C[C@@H]6C)CC5)cc4F)CC2)n3C)cc1F] |
| | 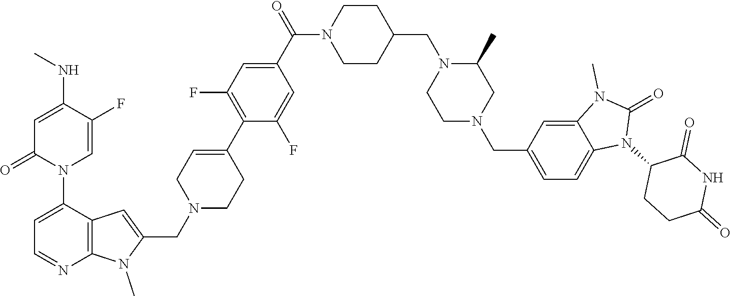 [SMILES: CNc1cc(=O)n(-c2ccnc3c2cc(CN2CC=C(c4c(F)cc(C(=O)N5CCC(CN6CCN(Cc7ccc8c(c7)n(C)c(=O)n8[C@H]7CCC(=O)NC7=O)C[C@@H]6C)CC5)cc4F)CC2)n3C)cc1F] |
| 3u57 | 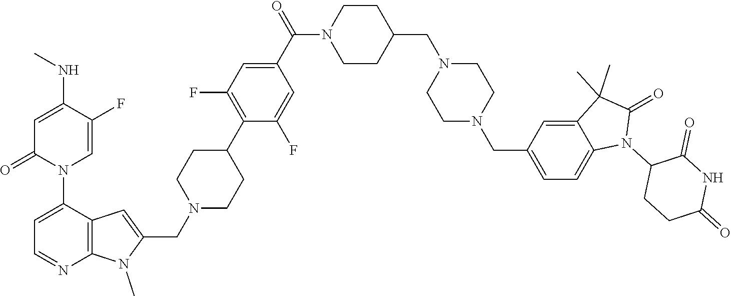 [SMILES: CNc1cc(=O)n(-c2ccnc3c2cc(CN2CCC(c4c(F)cc(C(=O)N5CCC(CN6CCN(Cc7ccc8c(c7)C(C)(C)C(=O)N8C7CCC(=O)NC7=O)CC6)CC5)cc4F)CC2)n3C)cc1F] |
| 3u58 and 3u59 | 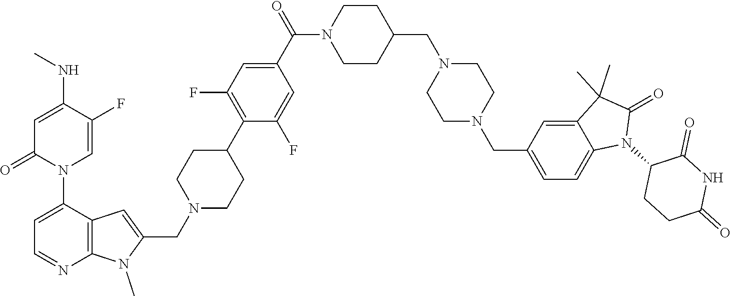 [SMILES: CNc1cc(=O)n(-c2ccnc3c2cc(CN2CCC(c4c(F)cc(C(=O)N5CCC(CN6CCN(Cc7ccc8c(c7)C(C)(C)C(=O)N8[C@H]7CCC(=O)NC7=O)CC6)CC5)cc4F)CC2)n3C)cc1F] |
| | 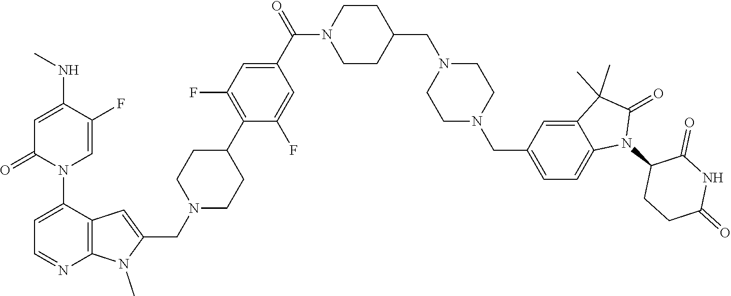 [SMILES: CNc1cc(=O)n(-c2ccnc3c2cc(CN2CCC(c4c(F)cc(C(=O)N5CCC(CN6CCN(Cc7ccc8c(c7)C(C)(C)C(=O)N8[C@@H]7CCC(=O)NC7=O)CC6)CC5)cc4F)CC2)n3C)cc1F] |
| 3u6 and 3u7 | 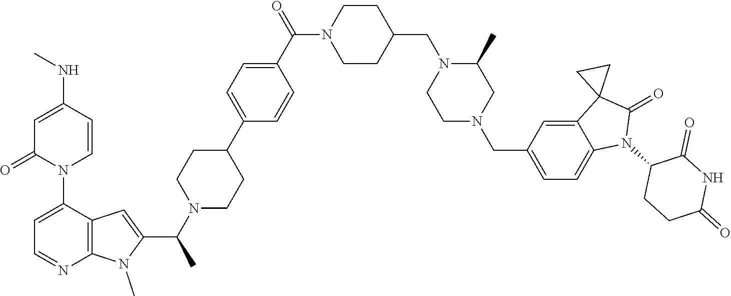 [SMILES: CNc1ccn(-c2ccnc3c2cc([C@H](C)N2CCC(c4ccc(C(=O)N5CCC(CN6CCN(Cc7ccc8c(c7)C7(CC7)C(=O)N8[C@H]7CCC(=O)NC7=O)C[C@@H]6C)CC5)cc4)CC2)n3C)c(=O)c1] |
| | 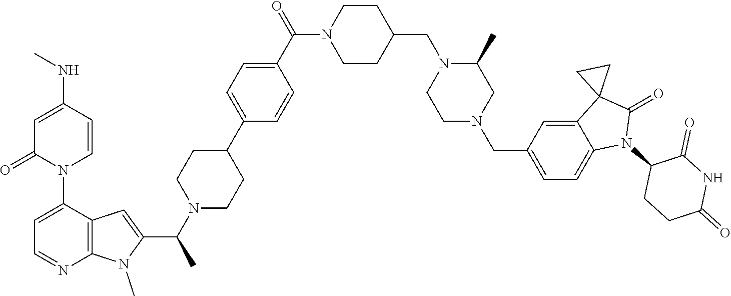 [SMILES: CNc1ccn(-c2ccnc3c2cc([C@H](C)N2CCC(c4ccc(C(=O)N5CCC(CN6CCN(Cc7ccc8c(c7)C7(CC7)C(=O)N8[C@@H]7CCC(=O)NC7=O)C[C@@H]6C)CC5)cc4)CC2)n3C)c(=O)c1] |
| 3u60 | 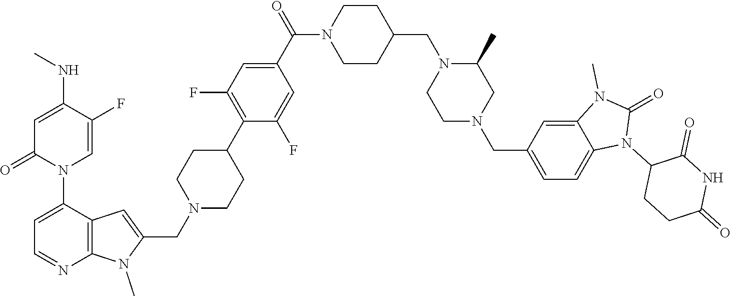 [SMILES: CNc1cc(=O)n(-c2ccnc3c2cc(CN2CCC(c4c(F)cc(C(=O)N5CCC(CN6CCN(Cc7ccc8c(c7)n(C)c(=O)n8C7CCC(=O)NC7=O)C[C@@H]6C)CC5)cc4F)CC2)n3C)cc1F] |
| 3u61 and 3u62 | 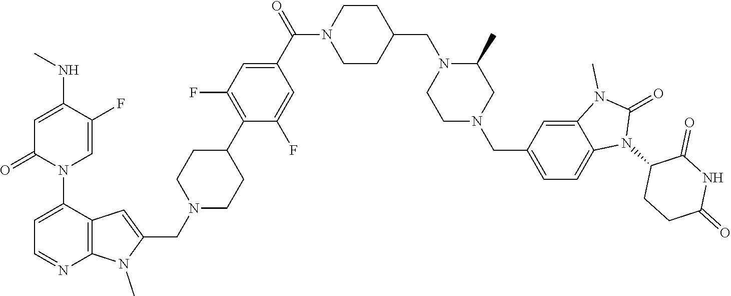 [SMILES: CNc1cc(=O)n(-c2ccnc3c2cc(CN2CCC(c4c(F)cc(C(=O)N5CCC(CN6CCN(Cc7ccc8c(c7)n(C)c(=O)n8[C@H]7CCC(=O)NC7=O)C[C@@H]6C)CC5)cc4F)CC2)n3C)cc1F] |
| | 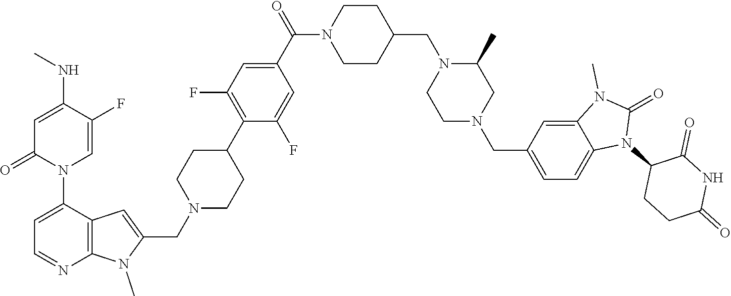 [SMILES: CNc1cc(=O)n(-c2ccnc3c2cc(CN2CCC(c4c(F)cc(C(=O)N5CCC(CN6CCN(Cc7ccc8c(c7)n(C)c(=O)n8[C@@H]7CCC(=O)NC7=O)C[C@@H]6C)CC5)cc4F)CC2)n3C)cc1F] |
| 3u63 | 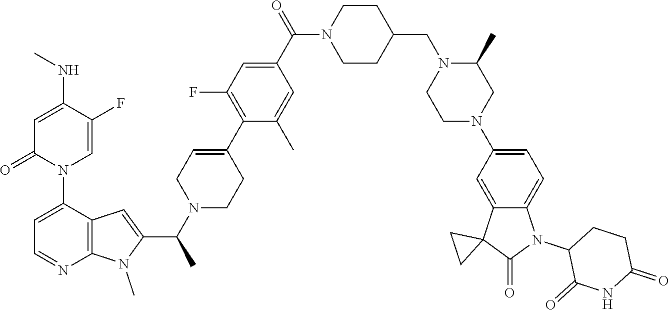 [SMILES: CNc1cc(=O)n(-c2ccnc3c2cc([C@H](C)N2CC=C(c4c(C)cc(C(=O)N5CCC(CN6CCN(c7ccc8c(c7)C7(CC7)C(=O)N8C7CCC(=O)NC7=O)C[C@@H]6C)CC5)cc4F)CC2)n3C)cc1F] |
| 3u64 and 3u65 | 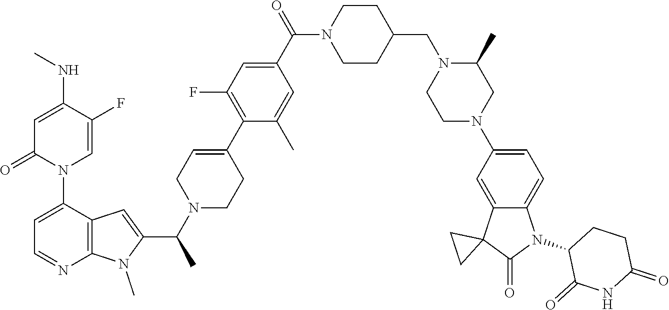 [SMILES: CNc1cc(=O)n(-c2ccnc3c2cc([C@H](C)N2CC=C(c4c(C)cc(C(=O)N5CCC(CN6CCN(c7ccc8c(c7)C7(CC7)C(=O)N8[C@@H]7CCC(=O)NC7=O)C[C@@H]6C)CC5)cc4F)CC2)n3C)cc1F] |
| | 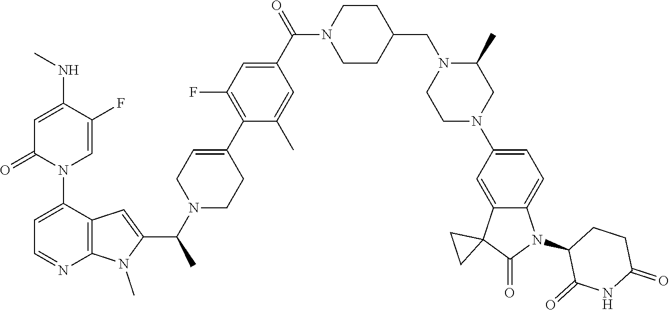 [SMILES: CNc1cc(=O)n(-c2ccnc3c2cc([C@H](C)N2CC=C(c4c(C)cc(C(=O)N5CCC(CN6CCN(c7ccc8c(c7)C7(CC7)C(=O)N8[C@H]7CCC(=O)NC7=O)C[C@@H]6C)CC5)cc4F)CC2)n3C)cc1F] |
| 3u66 | 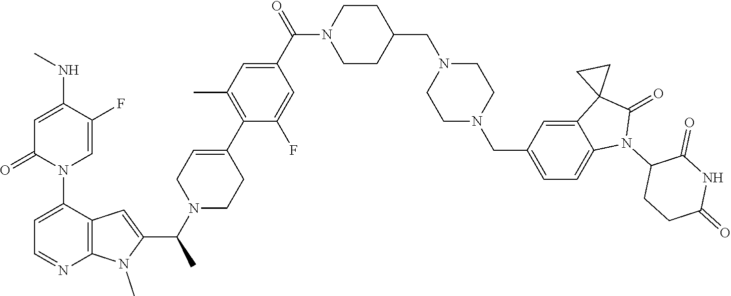 [SMILES: CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8C7CCC(=O)NC7=O)CC6)CC5)cc4F)CC2)n3C)cc1F] |
| 3u67 and 3u68 | 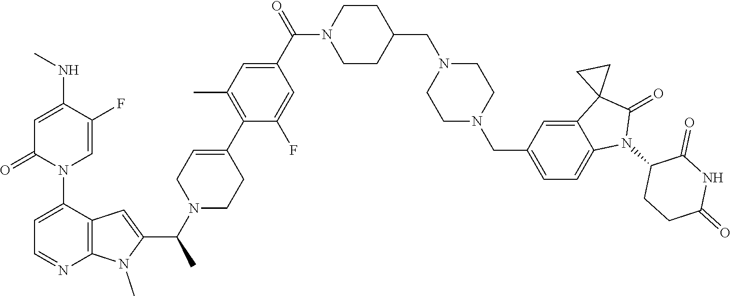 [SMILES: CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8[C@H]7CCC(=O)NC7=O)CC6)CC5)cc4F)CC2)n3C)cc1F] |
| | 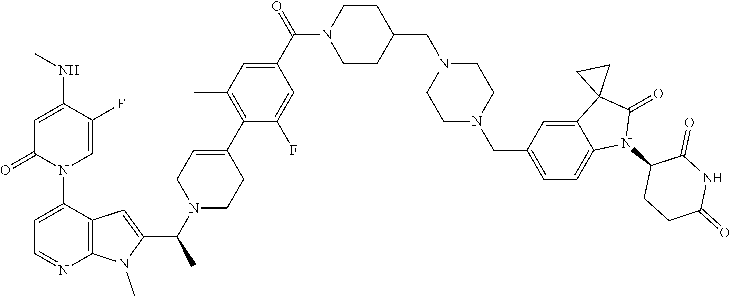 [SMILES: CNc1cc(=O)n(-c2ccnc3c2cc([C@H](C)N2CC=C(c4c(C)cc(C(=O)N5CCC(CN6CCN(Cc7ccc8c(c7)C7(CC7)C(=O)N8[C@@H]7CCC(=O)NC7=O)CC6)CC5)cc4F)CC2)n3C)cc1F] |
| 3u69 | 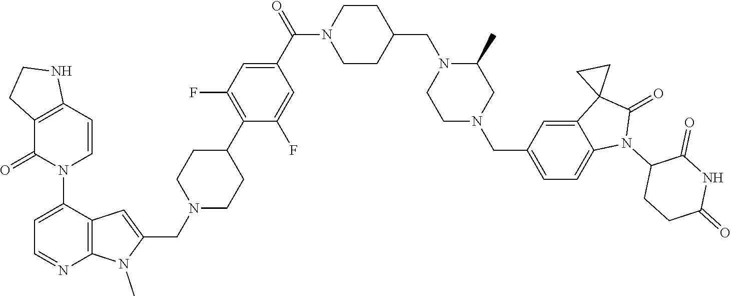 [SMILES: C[C@H]1CN(Cc2ccc3c(c2)C2(CC2)C(=O)N3C2CCC(=O)NC2=O)CCN1CC1CCN(C(=O)c2cc(F)c(C3CCN(Cc4cc5c(-n6ccc7c(c6=O)CCN7)ccnc5n4C)CC3)c(F)c2)CC1] |
| 3u70 | 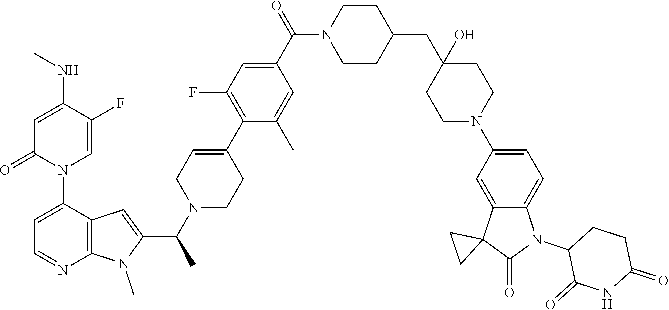 [SMILES: CNc1cc(=O)n(-c2ccnc3c2cc([C@H](C)N2CC=C(c4c(C)cc(C(=O)N5CCC(CC6(O)CCN(c7ccc8c(c7)C7(CC7)C(=O)N8C7CCC(=O)NC7=O)CC6)CC5)cc4F)CC2)n3C)cc1F] |
| 3u71 | 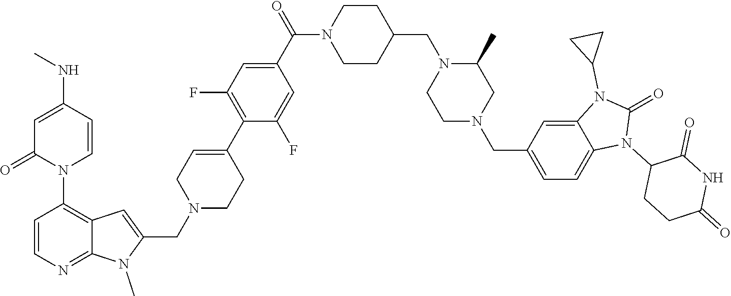 [SMILES: CNc1ccn(-c2ccnc3c2cc(CN2CC=C(c4c(F)cc(C(=O)N5CCC(CN6CCN(Cc7ccc8c(c7)n(C7CC7)c(=O)n8C7CCC(=O)NC7=O)C[C@@H]6C)CC5)cc4F)CC2)n3C)c(=O)c1] |
| 3u72 and 3u73 | 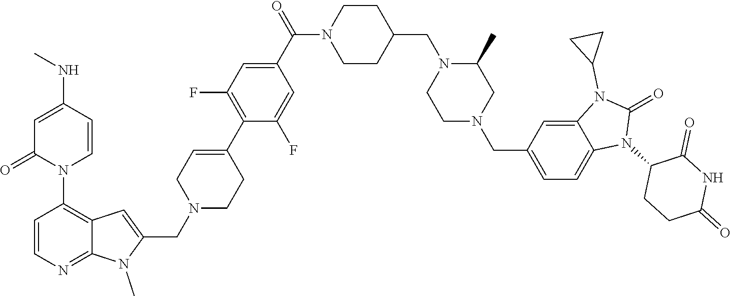 [SMILES: CNc1ccn(-c2ccnc3c2cc(CN2CC=C(c4c(F)cc(C(=O)N5CCC(CN6CCN(Cc7ccc8c(c7)n(C7CC7)c(=O)n8[C@H]7CCC(=O)NC7=O)C[C@@H]6C)CC5)cc4F)CC2)n3C)c(=O)c1] |
| | 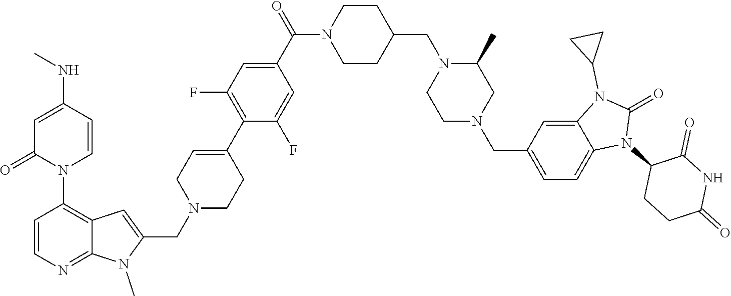 [SMILES: CNc1ccn(-c2ccnc3c2cc(CN2CC=C(c4c(F)cc(C(=O)N5CCC(CN6CCN(Cc7ccc8c(c7)n(C7CC7)c(=O)n8[C@@H]7CCC(=O)NC7=O)C[C@@H]6C)CC5)cc4F)CC2)n3C)c(=O)c1] |
| 3u74 | 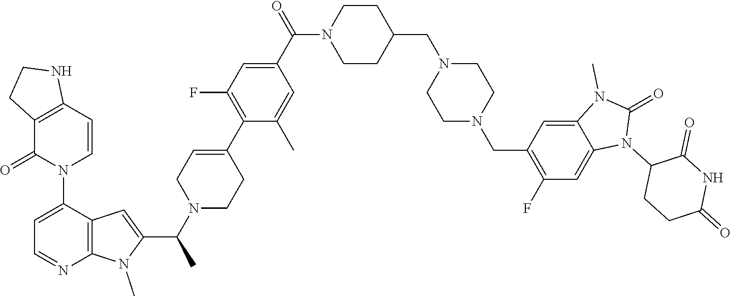 [SMILES: Cc1cc(C(=O)N2CCC(CN3CCN(Cc4cc5c(cc4F)n(C4CCC(=O)NC4=O)c(=O)n5C)CC3)CC2)cc(F)c1C1=CCN([C@@H](C)c2cc3c(-n4ccc5c(c4=O)CCN5)ccnc3n2C)CC1] |
| 3u75 and 3u76 | 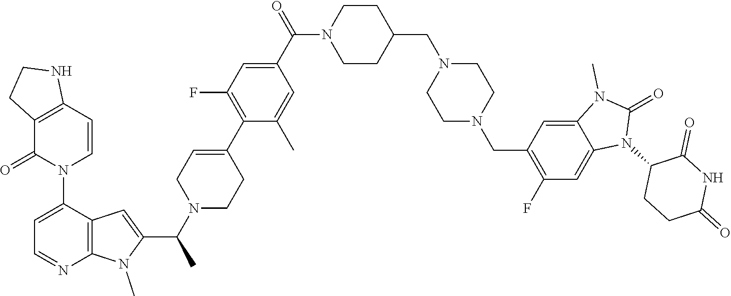 [SMILES: Cc1cc(C(=O)N2CCC(CN3CCN(Cc4cc5c(cc4F)n([C@H]4CCC(=O)NC4=O)c(=O)n5C)CC3)CC2)cc(F)c1C1=CCN([C@@H](C)c2cc3c(-n4ccc5c(c4=O)CCN5)ccnc3n2C)CC1] |
| | 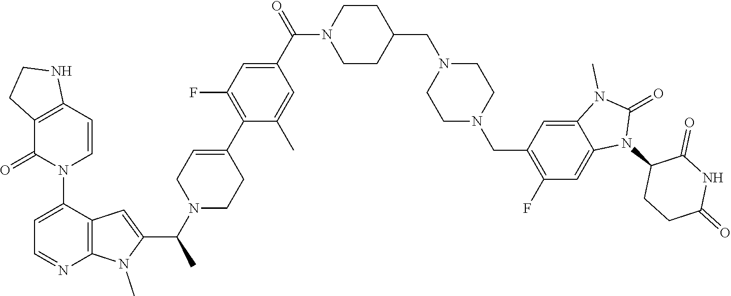 [SMILES: Cc1cc(C(=O)N2CCC(CN3CCN(Cc4cc5c(cc4F)n([C@@H]4CCC(=O)NC4=O)c(=O)n5C)CC3)CC2)cc(F)c1C1=CCN([C@@H](C)c2cc3c(-n4ccc5c(c4=O)CCN5)ccnc3n2C)CC1] |
| 3u77 | 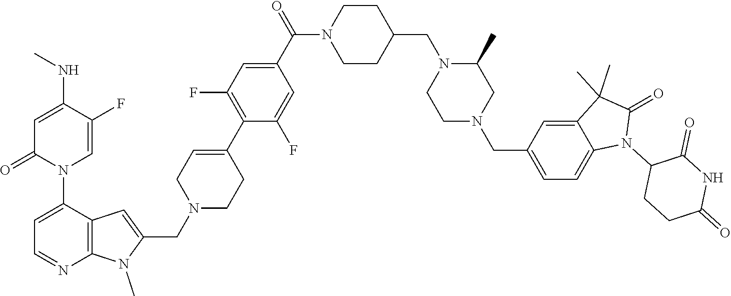 [SMILES: CNc1cc(=O)n(-c2ccnc3c2cc(CN2CC=C(c4c(F)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)cc1F] |
| 3u78 and 3u79 | 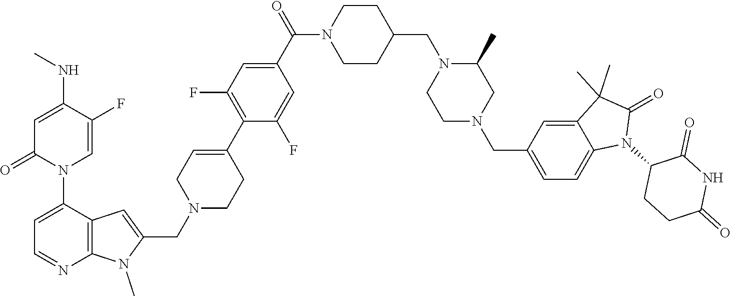 [SMILES: CNc1cc(=O)n(-c2ccnc3c2cc(CN2CC=C(c4c(F)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)cc1F] |
| | 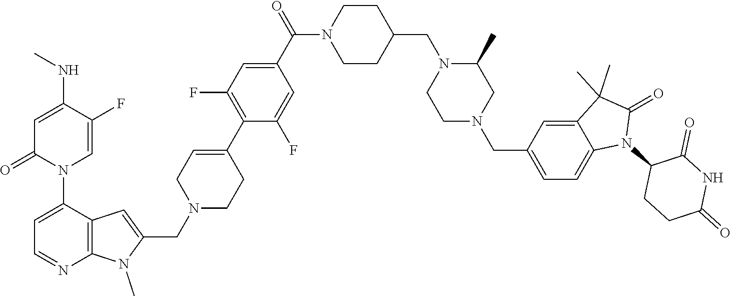 [SMILES: CNc1cc(=O)n(-c2ccnc3c2cc(CN2CC=C(c4c(F)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)cc1F] |
| 3u8 | 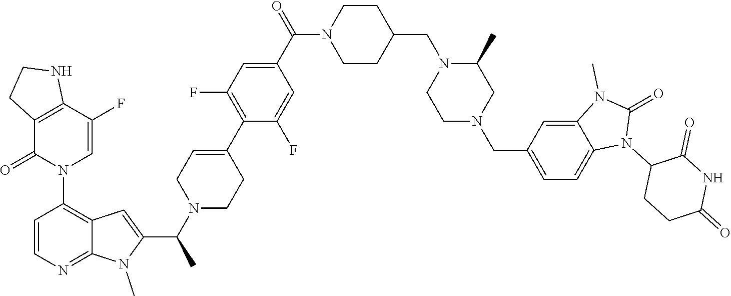 [SMILES: C[C@H]1CN(Cc2ccc3c(c2)n(C)c(=O)n3C2CCC(=O)NC2=O)CCN1CC1CCN(C(=O)c2cc(F)c(C3=CCN([C@@H](C)c4cc5c(-n6cc(F)c7c(c6=O)CCN7)ccnc5n4C)CC3)c(F)c2)CC1] |
| 3u80 | 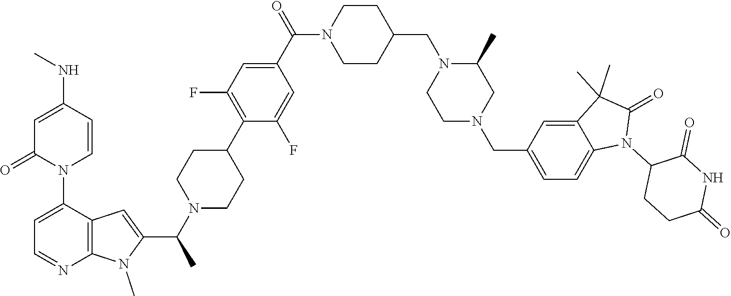 [SMILES: CNc1ccn(-c2ccnc3c2cc([C@H](C)N2CCC(c4c(F)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)c(=O)c1] |
| 3u81 and 3u82 | 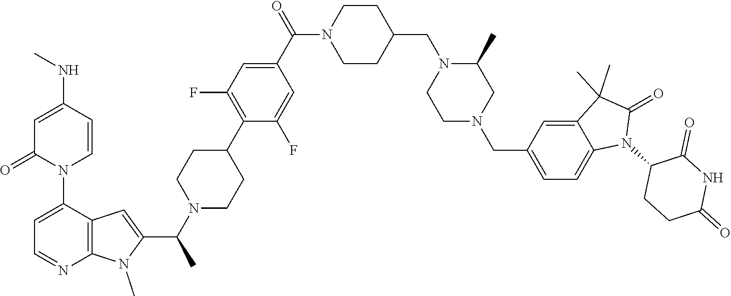 [SMILES: CNc1ccn(-c2ccnc3c2cc([C@H](C)N2CCC(c4c(F)cc(C(=O)N5CCC(CN6CCN(Cc7ccc8c(c7)C(C)(C)C(=O)N8[C@H]7CCC(=O)NC7=O)C[C@@H]6C)CC5)cc4F)CC2)n3C)c(=O)c1] |
| | 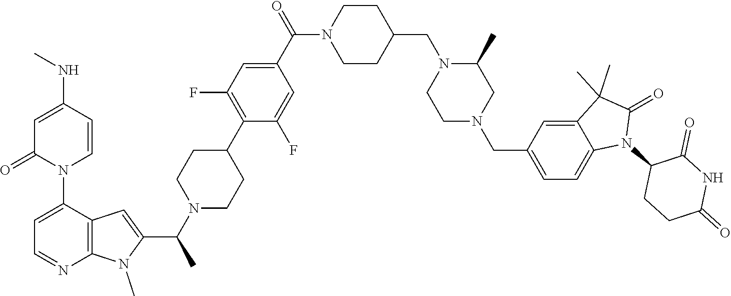 [SMILES: CNc1ccn(-c2ccnc3c2cc([C@H](C)N2CCC(c4c(F)cc(C(=O)N5CCC(CN6CCN(Cc7ccc8c(c7)C(C)(C)C(=O)N8[C@@H]7CCC(=O)NC7=O)C[C@@H]6C)CC5)cc4F)CC2)n3C)c(=O)c1] |
| 3u83 | 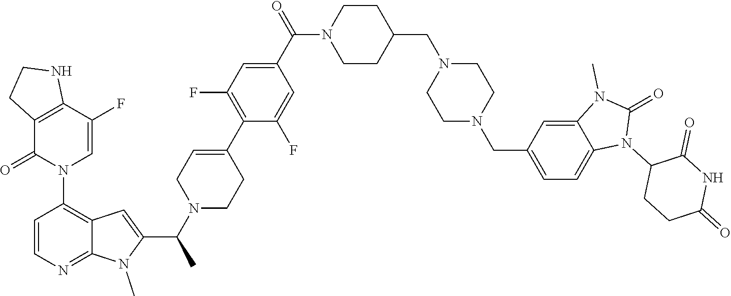 [SMILES: C[C@@H](c1cc2c(-n3cc(F)c4c(c3=O)CCN4)ccnc2n1C)N1CC=C(c2c(F)cc(C(=O)N3CCC(CN4CCN(Cc5ccc6c(c5)n(C)c(=O)n6C5CCC(=O)NC5=O)CC4)CC3)cc2F)CC1] |
| 3u84 | 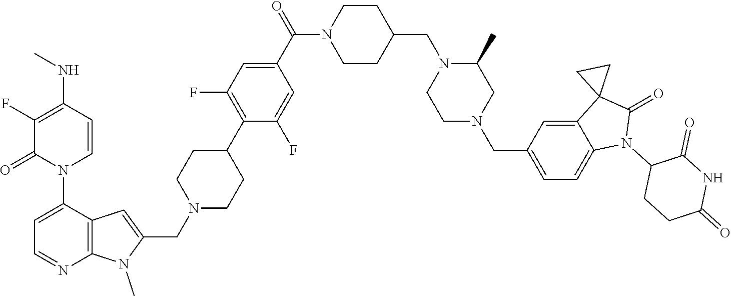 [SMILES: CNc1ccn(-c2ccnc3c2cc(CN2CCC(c4c(F)cc(C(=O)N5CCC(CN6CCN(Cc7ccc8c(c7)C7(CC7)C(=O)N8C7CCC(=O)NC7=O)C[C@@H]6C)CC5)cc4F)CC2)n3C)c(=O)c1F] |
| 3u85 | 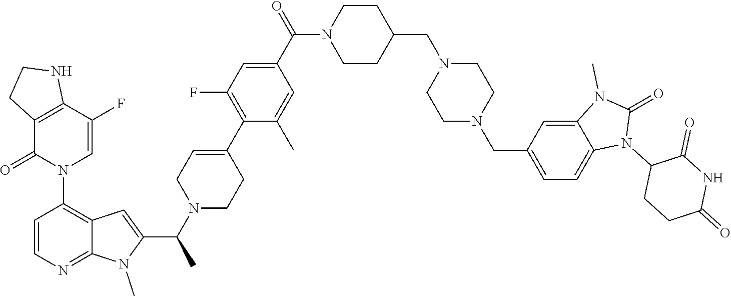 [SMILES: Cc1cc(C(=O)N2CCC(CN3CCN(Cc4ccc5c(c4)n(C)c(=O)n5C4CCC(=O)NC4=O)CC3)CC2)cc(F)c1C1=CCN([C@@H](C)c2cc3c(-n4cc(F)c5c(c4=O)CCN5)ccnc3n2C)CC1] |
| 3u86 | 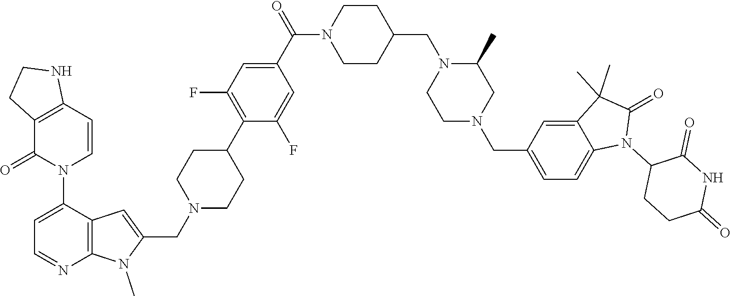 [SMILES: C[C@H]1CN(Cc2ccc3c(c2)C(C)(C)C(=O)N3C2CCC(=O)NC2=O)CCN1CC1CCN(C(=O)c2cc(F)c(C3CCN(Cc4cc5c(-n6ccc7c(c6=O)CCN7)ccnc5n4C)CC3)c(F)c2)CC1] |
| 3u87 | 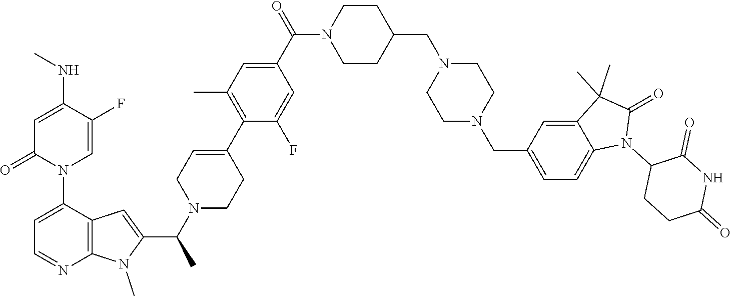 [SMILES: CNc1cc(=O)n(-c2ccnc3c2cc([C@H](C)N2CC=C(c4c(C)cc(C(=O)N5CCC(CN6CCN(Cc7ccc8c(c7)C(C)(C)C(=O)N8C7CCC(=O)NC7=O)CC6)CC5)cc4F)CC2)n3C)cc1F] |
| 3u88 and 3u89 | 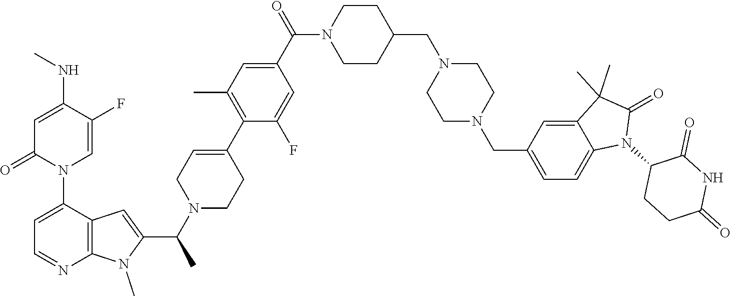 [SMILES: CNc1cc(=O)n(-c2ccnc3c2cc([C@H](C)N2CC=C(c4c(C)cc(C(=O)N5CCC(CN6CCN(Cc7ccc8c(c7)C(C)(C)C(=O)N8[C@H]7CCC(=O)NC7=O)CC6)CC5)cc4F)CC2)n3C)cc1F] |
| | 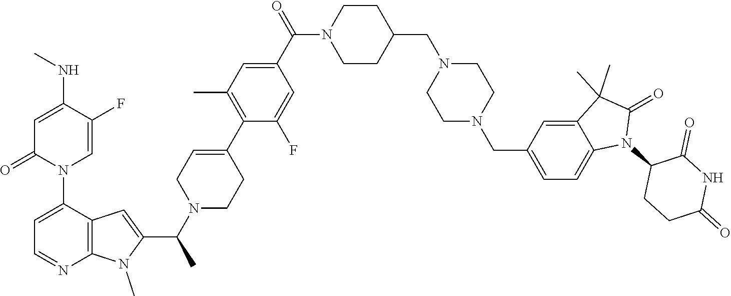 [SMILES: CNc1cc(=O)n(-c2ccnc3c2cc([C@H](C)N2CC=C(c4c(C)cc(C(=O)N5CCC(CN6CCN(Cc7ccc8c(c7)C(C)(C)C(=O)N8[C@@H]7CCC(=O)NC7=O)CC6)CC5)cc4F)CC2)n3C)cc1F] |
| 3u9 | 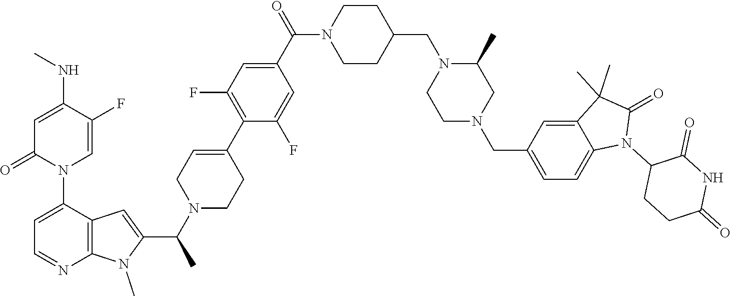 [SMILES: CNc1cc(=O)n(-c2ccnc3c2cc([C@H](C)N2CC=C(c4c(F)cc(C(=O)N5CCC(CN6CCN(Cc7ccc8c(c7)C(C)(C)C(=O)N8C7CCC(=O)NC7=O)C[C@@H]6C)CC5)cc4F)CC2)n3C)cc1F] |
| 3u90 | 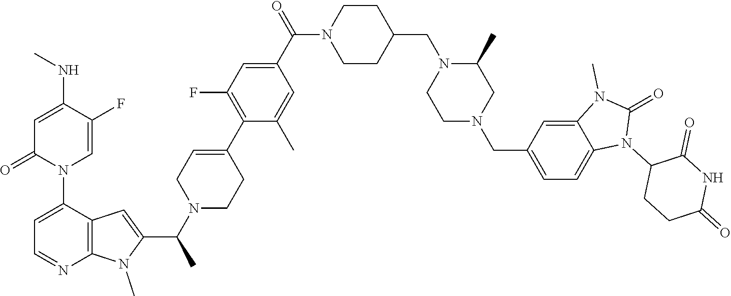 [SMILES: CNc1cc(=O)n(-c2ccnc3c2cc([C@H](C)N2CC=C(c4c(C)cc(C(=O)N5CCC(CN6CCN(Cc7ccc8c(c7)n(C)c(=O)n8C7CCC(=O)NC7=O)C[C@@H]6C)CC5)cc4F)CC2)n3C)cc1F] |
| 3u91 and 3u92 | 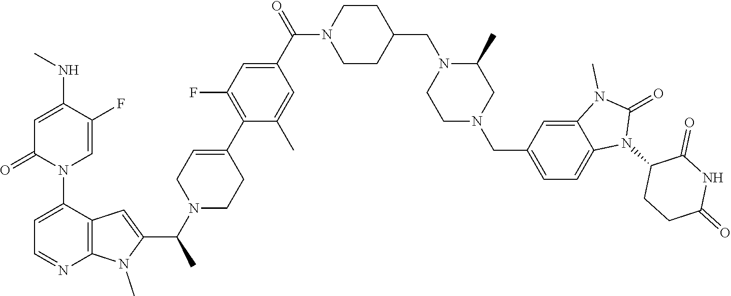 [SMILES: CNc1cc(=O)n(-c2ccnc3c2cc([C@H](C)N2CC=C(c4c(C)cc(C(=O)N5CCC(CN6CCN(Cc7ccc8c(c7)n(C)c(=O)n8[C@H]7CCC(=O)NC7=O)C[C@@H]6C)CC5)cc4F)CC2)n3C)cc1F] |
| | 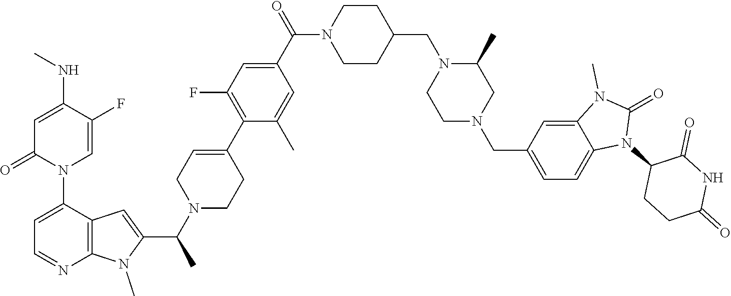 [SMILES: CNc1cc(=O)n(-c2ccnc3c2cc([C@H](C)N2CC=C(c4c(C)cc(C(=O)N5CCC(CN6CCN(Cc7ccc8c(c7)n(C)c(=O)n8[C@@H]7CCC(=O)NC7=O)C[C@@H]6C)CC5)cc4F)CC2)n3C)cc1F] |
| 3u93 | 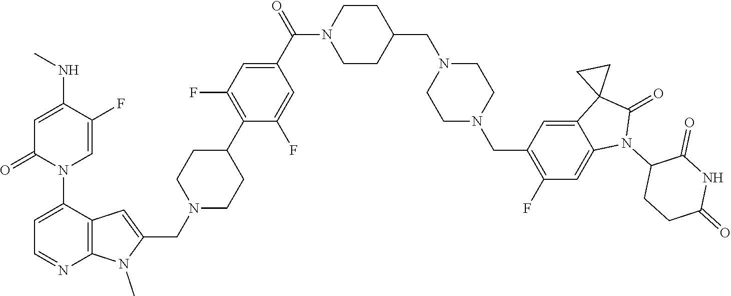 [SMILES: CNc1cc(=O)n(-c2ccnc3c2cc(CN2CCC(c4c(F)cc(C(=O)N5CCC(CN6CCN(Cc7cc8c(cc7F)N(C7CCC(=O)NC7=O)C(=O)C87CC7)CC6)CC5)cc4F)CC2)n3C)cc1F] |
| 3u94 and 3u95 | 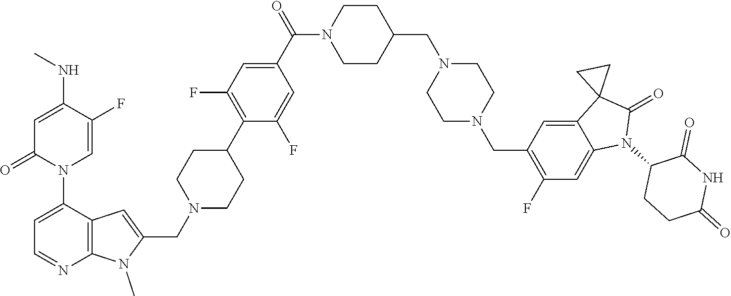 [SMILES: CNc1cc(=O)n(-c2ccnc3c2cc(CN2CCC(c4c(F)cc(C(=O)N5CCC(CN6CCN(Cc7cc8c(cc7F)N([C@H]7CCC(=O)NC7=O)C(=O)C87CC7)CC6)CC5)cc4F)CC2)n3C)cc1F] |
| | 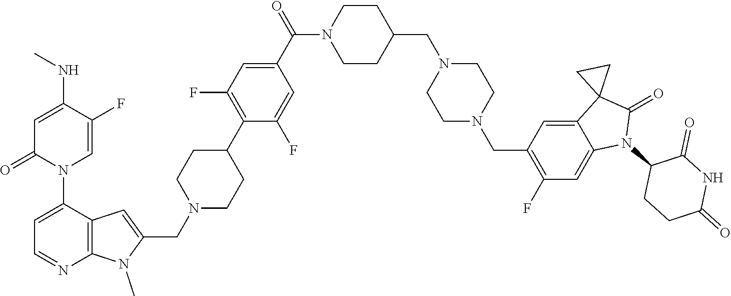 [SMILES: CNc1cc(=O)n(-c2ccnc3c2cc(CN2CCC(c4c(F)cc(C(=O)N5CCC(CN6CCN(Cc7cc8c(cc7F)N([C@@H]7CCC(=O)NC7=O)C(=O)C87CC7)CC6)CC5)cc4F)CC2)n3C)cc1F] |
| 3u96 | 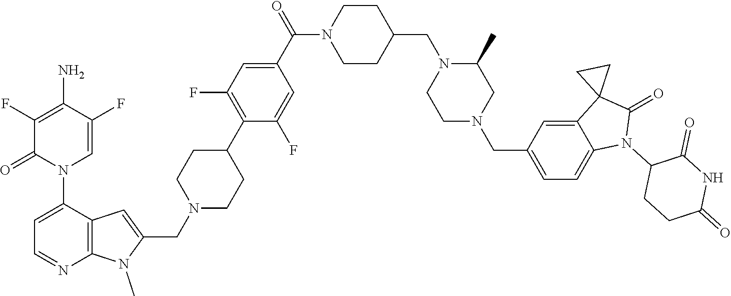 [SMILES: C[C@H]1CN(Cc2ccc3c(c2)C2(CC2)C(=O)N3C2CCC(=O)NC2=O)CCN1CC1CCN(C(=O)c2cc(F)c(C3CCN(Cc4cc5c(-n6cc(F)c(N)c(F)c6=O)ccnc5n4C)CC3)c(F)c2)CC1] |
| 3u97 and 3u98 | 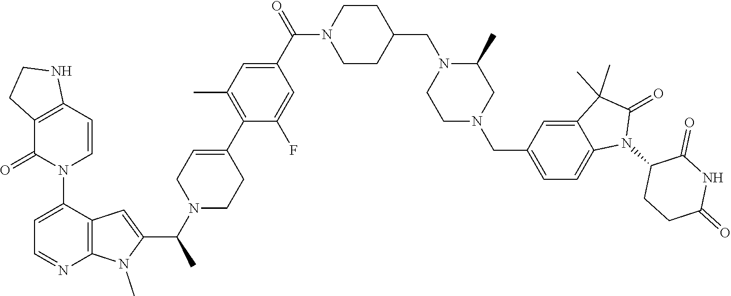 [SMILES: Cc1cc(C(=O)N2CCC(CN3CCN(Cc4ccc5c(c4)C(C)(C)C(=O)N5[C@H]4CCC(=O)NC4=O)C[C@@H]3C)CC2)cc(F)c1C1=CCN([C@@H](C)c2cc3c(-n4ccc5c(c4=O)CCN5)ccnc3n2C)CC1] |
| | 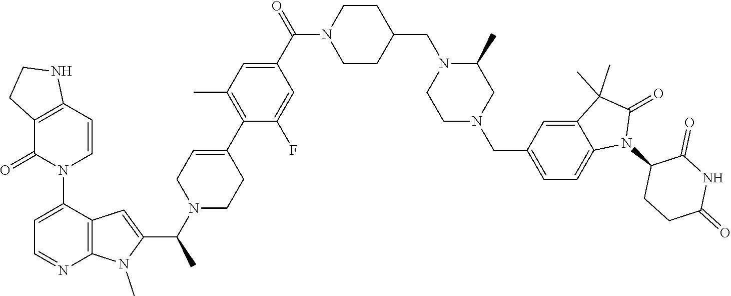 [SMILES: Cc1cc(C(=O)N2CCC(CN3CCN(Cc4ccc5c(c4)C(C)(C)C(=O)N5[C@@H]4CCC(=O)NC4=O)C[C@@H]3C)CC2)cc(F)c1C1=CCN([C@@H](C)c2cc3c(-n4ccc5c(c4=O)CCN5)ccnc3n2C)CC1] |
| 3h43 | 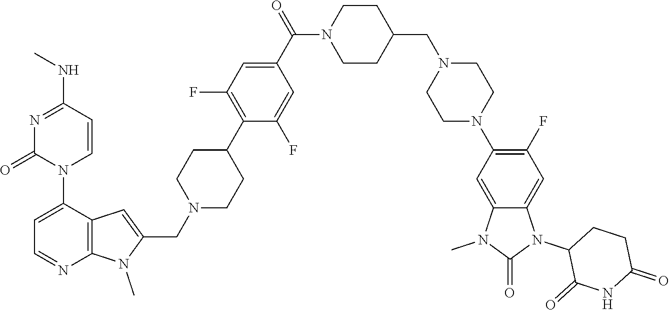 [SMILES: CNc1ccn(-c2ccnc3c2cc(CN2CCC(c4c(F)cc(C(=O)N5CCC(CN6CCN(c7cc8c(cc7F)n(C7CCC(=O)NC7=O)c(=O)n8C)CC6)CC5)cc4F)CC2)n3C)c(=O)n1] |

or a pharmaceutically acceptable salt or stereoisomer thereof.

35.-39. (canceled)

40. A pharmaceutical composition comprising a compound according to claim 1, or a pharmaceutically acceptable salt or stereoisomer thereof, together with a pharmaceutically acceptable vehicle or excipient or pharmaceutically acceptable carrier(s).

41.-42. (canceled)

43. A method of treating an immune mediated disease or condition in a subject in need thereof, the method comprising administering to the subject a therapeutically effective amount of a compound according to claim 1, or a pharmaceutically acceptable salt or stereoisomer thereof.

44.-45. (canceled)

46. A method of treating a disease or condition mediated by a signal transducer and activator of transcription 6 (STAT6) protein activity in a subject in need thereof, the method comprising administering to the subject a therapeutically effective amount of a compound according to claim 1, or a pharmaceutically acceptable salt or stereoisomer thereof optionally wherein the STAT6-mediated disorder or disease is rheumatoid arthritis (RA), systemic lupus erythematosus (SLE), lupus nephritis (LN), osteoarthritis (OA), ulcerative colitis (UC), Crohn's disease (CD), idiopathic pulmonary fibrosis (IPF), interstitial lung disease (ILD), metabolic dysfunction-associated steatohepatitis (MASH), Diabetic kidney disease (DKD) (diabetic nephropathy), or atopic dermatitis (AD).

47.-50. (canceled)

51. A method of treating a disease or condition mediated by interleukin 4 (IL-4) or interleukin 3 (IL-3) in a subject in need thereof, the method comprising administering to the subject a therapeutically effective amount of a compound according to claim 1, or a pharmaceutically acceptable salt or stereoisomer thereof.

52. (canceled)

53. A method of treating a disease or condition characterized by Th2-mediated inflammation, the method comprising administering to the subject a therapeutically effective amount of a compound of claim 1, or a pharmaceutically acceptable salt or stereoisomer thereof, optionally wherein the disease or condition is selected from atopic dermatitis, asthma, chronic rhinosinusitis with nasal polyposis, urticaria, rhinitis, eosinophilic esophagitis, food allergy, diffuse cutaneous systemic sclerosis, alopecia areata and/or COPD (chronic obstructive pulmonary disease) and different cancers such as lymphomas, triple negative breast cancer and solid fibrous cancers either as a stand-alone treatment or in combination with other anticancer drugs such as check point inhibitors.
